(19) United States
(12) Patent Application Publication
(10) Pub. No.: US 2023/0339886 A1
(43) Pub. Date: Oct. 26, 2023

(54) REV-ERB AGONISTS FOR THE TREATMENT OF TH17-MEDIATED INFLAMMATORY DISORDERS

(71) Applicants: University of Florida Research Foundation, Incorporated, Gainesville, FL (US); SHANGPHARMA INNOVATION INC., South San Francisco, CA (US)

(72) Inventors: Theodore Mark Kamenecka, Gainesville, FL (US); Kevin Greenman, Sunnyvale, CA (US); Laura Solt, Gainesville, FL (US); Yuanjun He, Gainesville, FL (US); Mike Lizarzaburu, Pacifica, CA (US); Brett C. Bookser, Kensington, CA (US)

(73) Assignees: University of Florida Research Foundation, Incorporated, Gainesville, CA (US); SHANGPHARMA INNOVATION INC., South San Francisco, CA (US)

(21) Appl. No.: 18/012,471

(22) PCT Filed: Jun. 23, 2021
(86) PCT No.: PCT/US2021/070763

§ 371 Date: Dec. 22, 2022

Related U.S. Application Data
(60) Provisional application No. 62/705,349, filed on Jun. 23, 2020.

(51) Int. Cl. C07D 401/10; C07D 401/14; C07D 209/08; C07D 209/14; C07D 471/04; C07D 403/10; C07D 401/04; C07D 413/10; C07D 405/10; C07D 403/12; C07D 417/10

(52) U.S. Cl. CPC C07D 401/10; C07D 401/14; C07D 209/08; C07D 209/14; C07D 471/04; C07D 403/10; C07D 401/04; C07D 413/10; C07D 405/10; C07D 403/12; C07D 417/10

(57) ABSTRACT

The present disclosure provides compounds of Formula IA and Formula IB and their pharmaceutical compositions as selective agonists of REV-ERB-α: where R¹, R², R³, R⁴, R⁵, R^{X1}, R^{X2}, n^{A}, n^{B}, X, Y, and Z are described herein. The compounds are useful in various methods and uses, such as in the treatment of diseases including hyperglycemia, dyslipidemia, atherosclerosis, and autoimmune and inflammatory disorders or diseases, and as cancer therapeutics, such as for the treatment of glioblastoma, hepatocellular carcinoma, and colorectal cancer, and for immune-oncology purposes.

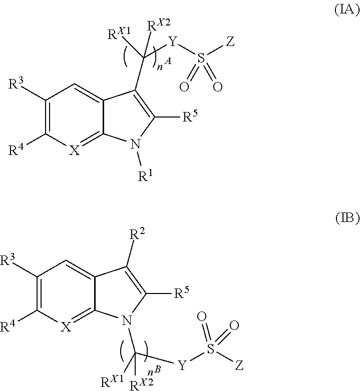

The present application claims the benefit of priority to U.S. Provisional Patent Application No. 62/705,349 filed on Jun. 23, 2020, which application is incorporated as if fully set forth herein.

### BACKGROUND

T_{H}17 cells are a subset of CD4⁺ T helper cells that preferentially secrete IL-17A, IL-17F, IL-21, and IL-22, and are important during tissue inflammation and anti-microbial/anti-fungal immunity (McGeachy, M. J. et al. (2008) Immunity 28, 445-453). Under homeostatic conditions, T_{H}17 cells have essential roles in protective immunity against extracellular pathogens at mucosal barriers (McGeachy, 2008). However, T_{H}17 cells also are implicated in the pathogenesis of several autoimmune diseases, including multiple sclerosis and psoriasis (Cho, J. H. (2008) Nat Rev Immunol 8, 458-466; Lees, C. W. et al. (2011) Gut 60, 1739-1753; Nair, R. P. et al. (2009) Nat Genet 41, 199-204), indicating that failure of T_{H}17 cell homeostasis can give rise to disease.

A number of studies have identified factors that drive T_{H}17 cell development and pathogenicity, including both the nuclear receptors retinoic-acid-receptor related orphan receptor α and γt (Ivanov, I I et al. (2006) Cell 126, 1121-1133; Yang, X. O. et al. (2008) Immunity 28, 29-39). RORγt is considered the lineage defining transcription factor regulating T_{H}17 cell development and a considerable amount of research has elucidated RORγt's genomic functions. Two other members of the nuclear receptor superfamily, REV-ERBα (NR1D1) and REV-ERBβ (NR1D2), are often co-expressed in the same tissues as the RORs and bind the same DNA response elements resulting in mutual cross talk and co-regulation of their shared target genes (Kojetin, D. J. et al. (2014) Nat Rev Drug Discov 13, 197-216).

Outside of the immune system, the RORs and the REV-ERBs modulate a number of physiological processes, but are best known for their roles in the regulation of the circadian rhythm, lipid, and glucose metabolic processes. The REV-ERBs are unique within the nuclear receptor superfamily in that they lack the carboxy-terminal tail of their ligand binding domain (LBD) called the activation function 2 region (AF-2, helix 12), which is required for coactivator recognition. Thus, in contrast to the RORs, which are constitutive activators of transcription, the REV-ERBs are transcriptional repressors (Kojetin, 2014). Collectively, the balance of expression of the RORs and REV-ERBs is critical for dynamic regulation of their target genes (Kojetin, 2014).

Most members of the nuclear receptor superfamily are ligand-regulated transcription factors and represent attractive therapeutic targets, including RORγt. After the initial identification of several synthetic RORγ modulators, including the compounds SR1001 and digoxin (Huh, J. R. et al. (2011) Nature 472, 486-490; Solt, L. A. et al. (2011) Nature 472, 491-494), additional RORγ ligands were identified, demonstrating the tractability of RORγt-targeted treatment of T_{H}17-mediated autoimmunity (Bronner, S. M. et al. (2017) Expert Opin Ther Pat 27, 101-112).

The REV-ERBs are also ligand-regulated transcription factors: the porphyrin heme was identified as the endogenous ligand for both REV-ERBα and REV-ERBβ (Raghuram, S. et al. (2007) Nat Struct Mol Biol 14, 1207-1213; Yin, L. et al. (2007) Science 318, 1786-1789). Synthetic ligands also can modulate the activity of the REV-ERBs' both in vitro and in vivo (Banerjee, S. et al. (2014) Nat Commun 5, 5759; Kojetin, D. et al. (2011) ACS Chem Biol 6, 131-134; Solt, L. A. et al. (2012) Nature 485, 62-68). For example, use of the compounds SR9009 and SR9011, which selectively target the REV-ERBs, demonstrated that in vivo pharmacological modulation of REV-ERB activity affected REV-ERB-mediated processes, including regulation of the circadian rhythm, glucose, and lipid metabolic processes (Solt et al., 2012). Despite the well-documented overlap in genetic programs between the RORs and REV-ERBs in tissues outside of the immune system (Kojetin, 2014), the role for the REV-ERBs in T_{H}17 cell development is still poorly understood.

REV-ERBα was previously demonstrated to diurnally regulate T_{H}17 cell frequencies in vivo (Yu, W. et al. (2002) Biochem Biophys Res Commun 290, 933-941), and its function in the context of pro-inflammatory settings and autoimmunity is becoming more defined. REV-ERBα is expressed during T_{H}17 cell development and its presence is required for dampening T_{H}17-mediated pro-inflammatory cytokine expression (M. Amir et al., Cell Reports 25 (2018) 3733-3749). Overexpression of REV-ERBα suppressed T_{H}17 cell development whereas genetic deletion of REV-ERBα resulted in enhanced T_{H}17 cell development in vitro and exacerbated autoimmune responses in vivo (id). While REV-ERBα directly repressed Nfil3 (Yu, X. et al. (2013) Science 342, 727-730), it also competed with RORγt for binding at the Il17a promoter and CNS-5 enhancer region. Further, REV-ERBα binds within the Rorc promoter region, indicating potential cross-talk and autoregulation amongst these receptors for controlling T_{H}17 cytokine expression (Amir et al., 2018). REV-ERB-specific small molecules suppressed T_{H}17 cell development in vitro and the development of T_{H}17-mediated autoimmunity in vivo. They were effective when used in a “treatment mode” in several models of autoimmunity and chronic inflammation, demonstrating that REV-ERBα can function outside of its classical role as a core member of the circadian clock under pro-inflammatory conditions and is a cell-intrinsic negative regulator of T_{H}17 cell pro-inflammatory immune responses (Amir et al., 2018).

Pharmacological agonism of REV-ERBα has been shown to suppress hepatic fibrosis and inflammatory response in a non-alcoholic steatohepatitis (NASH) mouse model, indicating broader efficacy in treating non-alcoholic fatty liver disease (NAFLD), slowing its progression to NASH, and treating its component obesity, insulin resistance, and cardiovascular diseases (Griffett K. et al. (2020) PLoS ONE 15(10): e0236000), such as atherosclerosis (S. Sitaula et al. (2015) Biochemical and Biophysical Research Communications 460(3) 566-571). Activation of REV-ERBα is operative in treatment of hyperglycemia (Griffett, 2020) and in treating dyslipidemia and regulating inflammation to improve survival following acute myocardial infarction (Stujanna E N et al. (2017) PLoS ONE 12(12): e0189330). The anti-inflammatory properties of REV-ERBα agonism also indicate a pharmacological approach for treating allergic inflammation and asthma (E. Sturm et al. (2020) European Respiratory Journal (56): Suppl. 64, 2905).

Activation of REV-ERBα is an effective adversary to oncogenic processes, establishing pharmacological intervention for the treatment of cancers (Wagner P M et al. (2019) ASN Neuro.). REV-ERBα agonists can be effective as chemotherapeutics against tumor cell types including brain, leukemia, breast, colon, and melanoma (Sulli, G. et al. (2018) Nature 553: 351-355), and in treating small-cell lung cancer (W. Shen et al. (2020) Theranostics 10(1): 4466-4480).

### SUMMARY

Addressing these needs and others, the present disclosure provides in various embodiments a compound of Formula IA or IB, or a pharmaceutically acceptable salt thereof:

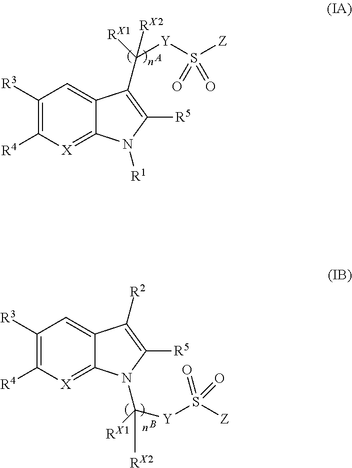

In Formula IA and IB, X is CH, CCl, CF, CBr, C(C₁-C₆-alkyl), or N.

R¹ is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S).

R² is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —C(O)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S), and wherein any alkyl, alkoxy, aryl, cycloalkyl, and heteroaryl in R¹ and R² is optionally substituted with 1 to 6 substituents selected from the group consisting of halo, NO₂, OH, CN, and C₁-C₆-haloalkyl.

R^{X1} and R^{X2} are independently selected from the group consisting of H, C₁-C₆-alkyl, C₁-C₆-haloalkyl, C₁-C₆-hydroxyalkyl, C₃-C₅-cycloalkyl, and —C(O)O(R′)₂ (wherein each R′ is independently selected from H and C₁-C₆-alkyl),
or R^{X1} and R^{X2} together with the carbon atom to which they are bound form a spiro-fused C₃-C₅-cycloalkyl;
or any two vicinal R^{X1} and R^{X2} together with the carbon atoms to which they are bound form a C₃-C₅-cycloalkyl or a moiety selected from

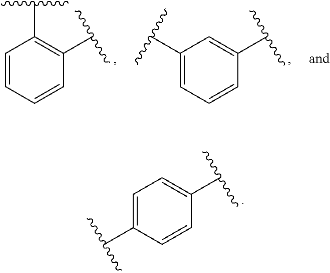 [SMILES: *c1ccccc1* ; *c1cccc(*)c1 ; *c1ccc(*)cc1]

Variable Y is —CR^{Y1}R^{Y2}— or —NR^{Y1}—; R^{Y1} and R^{Y2} are independently selected from H and C₁-C₆-alkyl; n^{A} is 0, 1, or 2; n^{B} is 1 or 2; and when Y is —NR^{Y1}—, then n^{A} is 1 or 2 and n^{B} is 2 and, optionally, R^{Y1} and one of R^{X1} and R^{X2}, together with the atoms to which they are bound, form a 3- to 6-membered heterocycloalkyl (wherein 1-4 ring members are independently selected from N, O, and S).

Variable Z is selected from the group consisting of C₁-C₆-alkyl, C₁-C₆-haloalkyl, C₃-C₅-cycloalkyl, 5- to 10-membered heteroaryl (wherein 1-4 heteroaryl members are independently selected from N, O, and S), 3- to 6-membered heterocycloalkyl (wherein 1-4 ring members are independently selected from N, O, and S), and —N^{Z1}R^{Z2}, wherein R^{Z1} and R^{Z2} are independently selected from the group consisting of H, C₁-C₆-alkyl and C₃-C₈-cycloalkyl, or R^{Z1} and R^{Z2} together with the nitrogen to which they are bound form a 3- to 6-membered heterocycloalkyl. Any alkyl, aryl, cycloalkyl, heterocycloalkyl, and heteroaryl in Z is optionally substituted with 1 to 6 substituents selected from the group consisting of halo, NO₂, OH, and C₁-C₆-haloalkyl.

Substituents R³ and R⁴ are independently selected from the group consisting of H, halo, CN, OH, N(R′)₂ (wherein each R′ is independently selected from H and C₁-C₆-alkyl), C₁-C₆-alkyl, C₁-C₆-haloalkyl, C₂-C₆-alkenyl, C₂-C₆-alkynyl, C₁-C₆-alkoxy, —O(C₁-C₆-alkyl)(C₆-C₁₀-aryl), C₃-C₈-cycloalkyl, C₆-C₁₀-aryl, and 5- to 10-membered heteroaryl (wherein 1-4 heteroaryl members are independently selected from N, O, and S), and 3- to 6-membered heterocycloalkyl (wherein 1-4 ring members are independently selected from N, O, and S), wherein R³ and R⁴ are not simultaneously H or simultaneously any combination of aryl, heteroaryl, and cycloalkyl.

R³ and R⁴ are optionally substituted with 1 to 3 substituents selected from the group consisting of halo, NO₂, OH, CN, C₁-C₆-alkyl, C₁-C₆-hydroxyalkyl, C₁-C₆-haloalkyl, —C₁-C₆-haloalkyl(OH), C₁-C₆-alkoxy, —S(O)₂C₁-C₆-alkyl, —S(O)₂NH(C₁-C₆-alkyl), —C(O)(C₁-C₆-alkyl), —C(O)O(C₁-C₆-alkyl), —C(O)N(R′)₂ (wherein each R′ is independently selected from H and C₁-C₆-alkyl), —NHC(O)R′, 3- to 6-membered heterocycloalkyl (wherein 1-4 ring members are independently selected from N, O, and S), and C₆-C₁₀-aryl.

In some embodiments, R³ and R⁴, together with the carbon atoms to which they are bound, form a fused C₅-C₆-cycloalkyl ring optionally substituted as defined for R³ and R⁴ herein.

Substituent R⁵ is selected from H and C₁-C₆-alkyl.

It should be understood that, notwithstanding the definitions provided herein, the compound of Formula IA or Formula IB does not include any of the following compounds:

| | |
|---|---|
| dimethyl({2-[3-(2-methylpropyl)-5-(pyridin-4-yl)- 1H-pyrrolo[2,3-b]pyridin-1- yl]ethyl}sulfamoyl)amine | 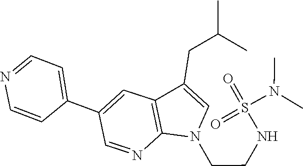 [SMILES: CC(C)Cc1cn(CCNS(=O)(=O)N(C)C)c2ncc(-c3ccncc3)cc12] |
| N-[2-(1,6-dimethyl-1H-indol-3-yl)ethyl]-3-methyl- 1-piperidinesulfonamide | 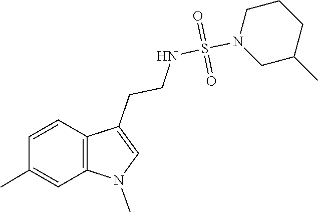 |
| N-[2-(1,6-dimethyl-1H-indol-3-yl)ethyl]-4-methyl- 1-piperidinesulfonamide | 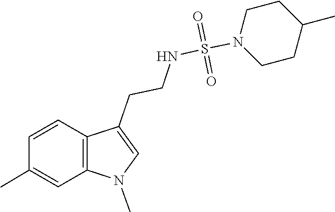 |
| N-[2-(1,6-dimethyl-1H-indol-3-yl)ethyl]-1- piperidinesulfonamide | 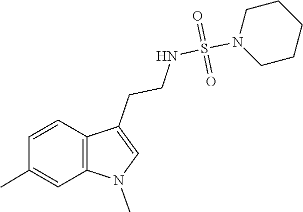 [SMILES: Cc1ccc2c(CCNS(=O)(=O)N3CCCCC3)cn(C)c2c1] |
| N-[2-(1,6-dimethyl-1H-indol-3-yl)ethyl]-1- pyrrolidinesulfonamide | 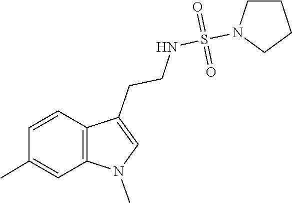 [SMILES: Cc1ccc2c(CCNS(=O)(=O)N3CCCC3)cn(C)c2c1] |
| N-[2-(1,6-dimethyl-1H-indol-3-yl)ethyl]-3-methyl- 1-pyrrolidinesulfonamide | 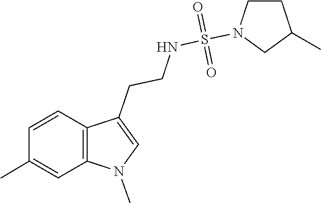 |
| N-[2-(1,6-dimethyl-1H-indol-3-yl)ethyl]-2-methyl- 1-piperidinesulfonamide | 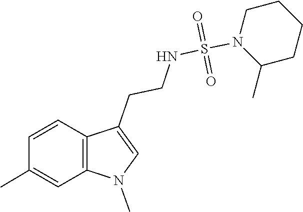 [SMILES: Cc1ccc2c(CCNS(=O)(=O)N3CCCCC3C)cn(C)c2c1] |
| N-[2-(1,6-dimethyl-1H-indol-3-yl)ethyl]-2- azabicyclo[2.2.1]heptane-2-sulfonamide | 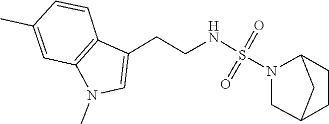 [SMILES: Cc1ccc2c(CCNS(=O)(=O)N3CC4CCC3C4)cn(C)c2c1] |
| N-[2-(1,6-dimethyl-1H-indol-3-yl)ethyl]-2-methyl- 1-pyrrolidinesulfonamide | 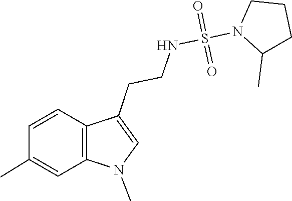 [SMILES: Cc1ccc2c(CCNS(=O)(=O)N3CCCC3C)cn(C)c2c1] |
| N-[2-(1,6-dimethyl-1H-indol-3-yl)ethyl]-4- morpholinesulfonamide | 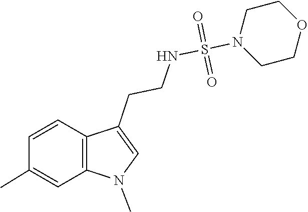 [SMILES: Cc1ccc2c(CCNS(=O)(=O)N3CCOCC3)cn(C)c2c1] |
| N-[2-(1,6-dimethyl-1H-indol-3-yl)ethyl]-2,6- dimethyl-4-morpholinesulfonamide | 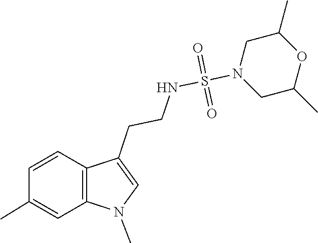 |
| N-[2-(1,6-dimethyl-1H-indol-3-yl)ethyl]-2,2- dimethyl-4-morpholinesulfonamide | 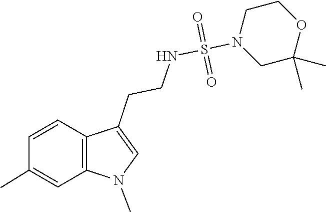 |
| 1-[[[2-(1,6-dimethyl-1H-indol-3- yl)ethyl]amino]sulfonyl]-4-Piperidinecarboxylic acid methyl ester | 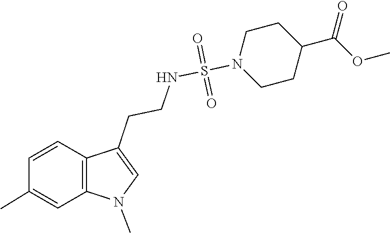 |

In various embodiments, the disclosure provides specific examples of Formula IA and Formula IB compounds, and their pharmaceutically acceptable salts, and/or tautomers thereof, and/or isotopologues thereof, as set forth in Tables 1A and 1B below.

TABLE 1A
Examples of Formula I Compounds

| | |
|---|---|
| 1 | 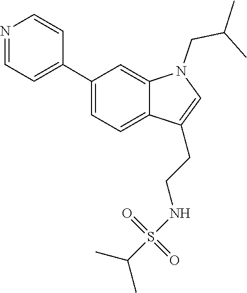 [SMILES: CC(C)Cn1cc(CCNS(=O)(=O)C(C)C)c2ccc(-c3ccncc3)cc21] |
| 2 | 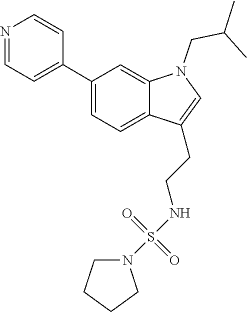 [SMILES: CC(C)Cn1cc(CCNS(=O)(=O)N2CCCC2)c2ccc(-c3ccncc3)cc21] |
| 3 | 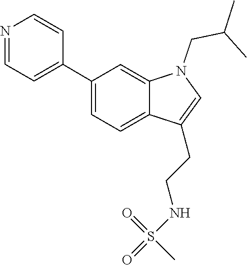 [SMILES: CC(C)Cn1cc(CCNS(C)(=O)=O)c2ccc(-c3ccncc3)cc21] |
| 5 | 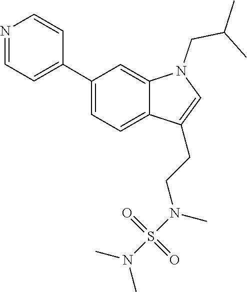 [SMILES: CC(C)Cn1cc(CCN(C)S(=O)(=O)N(C)C)c2ccc(-c3ccncc3)cc21] |
| 6 | 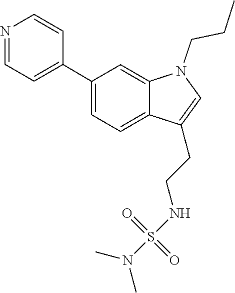 [SMILES: CCCn1cc(CCNS(=O)(=O)N(C)C)c2ccc(-c3ccncc3)cc21] |
| 7 | 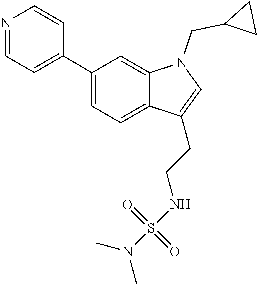 [SMILES: CN(C)S(=O)(=O)NCCc1cn(CC2CC2)c2cc(-c3ccncc3)ccc12] |
| 8 | 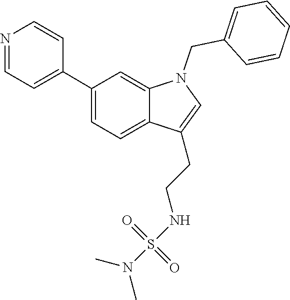 [SMILES: CN(C)S(=O)(=O)NCCc1cn(Cc2ccccc2)c2cc(-c3ccncc3)ccc12] |
| 9 | 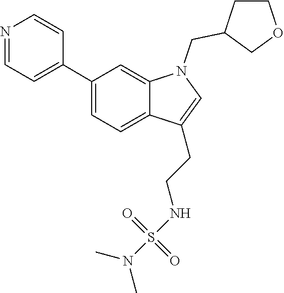 [SMILES: CN(C)S(=O)(=O)NCCc1cn(CC2CCOC2)c2cc(-c3ccncc3)ccc12] |
| 11 | 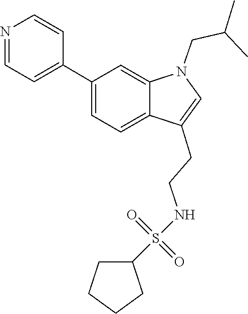 [SMILES: CC(C)Cn1cc(CCNS(=O)(=O)C2CCCC2)c2ccc(-c3ccncc3)cc21] |
| 12 | 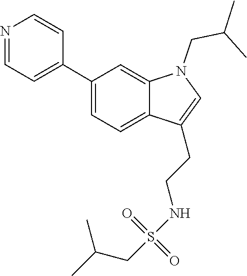 [SMILES: CC(C)Cn1cc(CCNS(=O)(=O)CC(C)C)c2ccc(-c3ccncc3)cc21] |
| 13 | 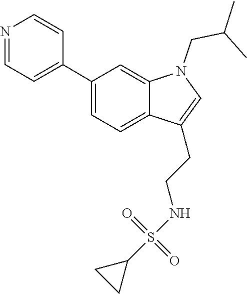 [SMILES: CC(C)Cn1cc(CCNS(=O)(=O)C2CC2)c2ccc(-c3ccncc3)cc21] |
| 14 | 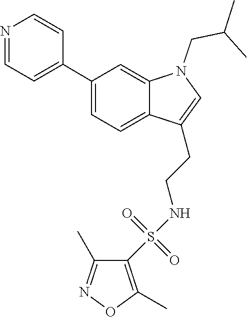 [SMILES: Cc1noc(C)c1S(=O)(=O)NCCc1cn(CC(C)C)c2cc(-c3ccncc3)ccc12] |
| 15 | 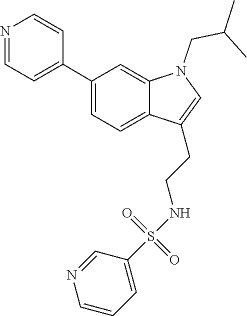 [SMILES: CC(C)Cn1cc(CCNS(=O)(=O)c2cccnc2)c2ccc(-c3ccncc3)cc21] |
| 16 | 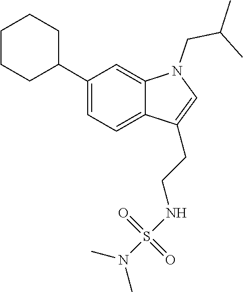 [SMILES: CC(C)Cn1cc(CCNS(=O)(=O)N(C)C)c2ccc(C3CCCCC3)cc21] |
| 17 | 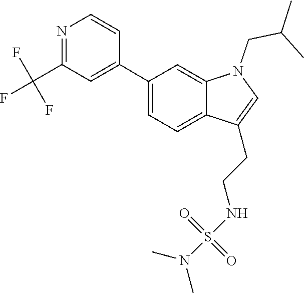 [SMILES: CC(C)Cn1cc(CCNS(=O)(=O)N(C)C)c2ccc(-c3ccnc(C(F)(F)F)c3)cc21] |
| 18 | 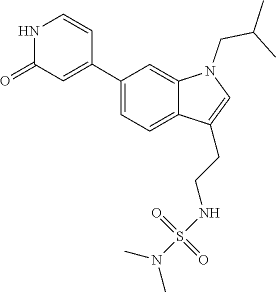 [SMILES: CC(C)Cn1cc(CCNS(=O)(=O)N(C)C)c2ccc(-c3cc[nH]c(=O)c3)cc21] |
| 19 | 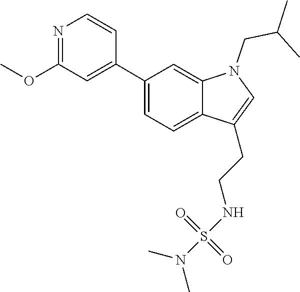 [SMILES: COc1cc(-c2ccc3c(CCNS(=O)(=O)N(C)C)cn(CC(C)C)c3c2)ccn1] |
| 20 | 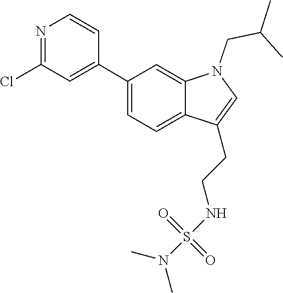 [SMILES: CC(C)Cn1cc(CCNS(=O)(=O)N(C)C)c2ccc(-c3ccnc(Cl)c3)cc21] |
| 21 | 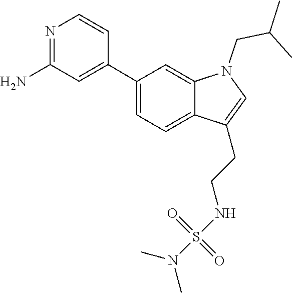 [SMILES: CC(C)Cn1cc(CCNS(=O)(=O)N(C)C)c2ccc(-c3ccnc(N)c3)cc21] |
| 22 | 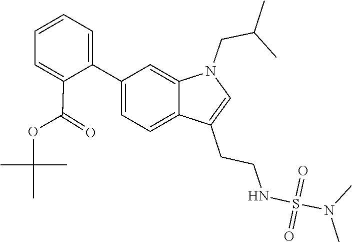 [SMILES: CC(C)Cn1cc(CCNS(=O)(=O)N(C)C)c2ccc(-c3ccccc3C(=O)OC(C)(C)C)cc21] |
| 23 | 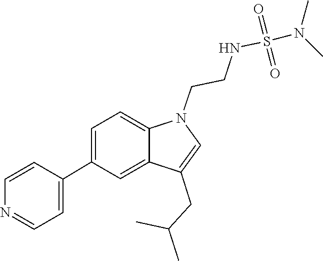 [SMILES: CC(C)Cc1cn(CCNS(=O)(=O)N(C)C)c2ccc(-c3ccncc3)cc12] |
| 24 | 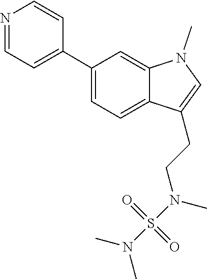 [SMILES: CN(C)S(=O)(=O)N(C)CCc1cn(C)c2cc(-c3ccncc3)ccc12] |
| 25 | 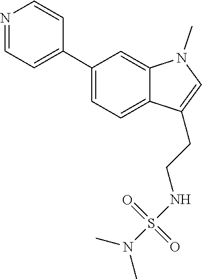 [SMILES: CN(C)S(=O)(=O)NCCc1cn(C)c2cc(-c3ccncc3)ccc12] |
| 26 | 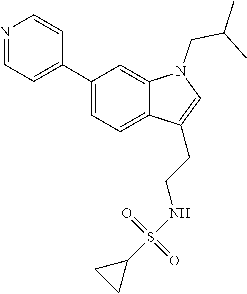 [SMILES: CC(C)Cn1cc(CCNS(=O)(=O)C2CC2)c2ccc(-c3ccncc3)cc21] |
| 27 | 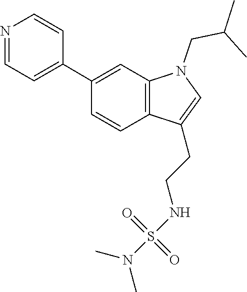 [SMILES: CC(C)Cn1cc(CCNS(=O)(=O)N(C)C)c2ccc(-c3ccncc3)cc21] |
| 28 | 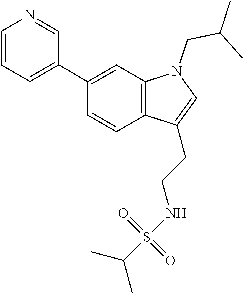 [SMILES: CC(C)Cn1cc(CCNS(=O)(=O)C(C)C)c2ccc(-c3cccnc3)cc21] |
| 29 | 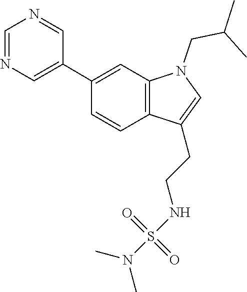 [SMILES: CC(C)Cn1cc(CCNS(=O)(=O)N(C)C)c2ccc(-c3cncnc3)cc21] |
| 30 | 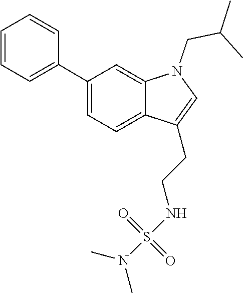 [SMILES: CC(C)Cn1cc(CCNS(=O)(=O)N(C)C)c2ccc(-c3ccccc3)cc21] |
| 31 | 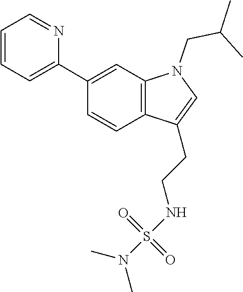 [SMILES: CC(C)Cn1cc(CCNS(=O)(=O)N(C)C)c2ccc(-c3ccccn3)cc21] |
| 32 | 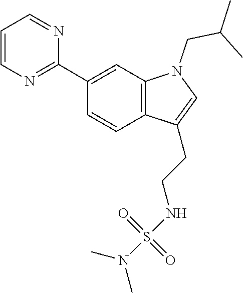 [SMILES: CC(C)Cn1cc(CCNS(=O)(=O)N(C)C)c2ccc(-c3ncccn3)cc21] |
| 33 | 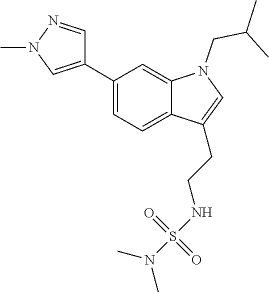 [SMILES: CC(C)Cn1cc(CCNS(=O)(=O)N(C)C)c2ccc(-c3cnn(C)c3)cc21] |
| 34 | 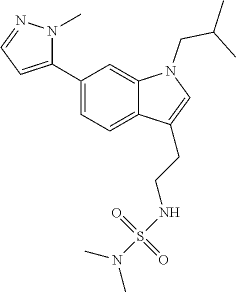 [SMILES: CC(C)Cn1cc(CCNS(=O)(=O)N(C)C)c2ccc(-c3ccnn3C)cc21] |
| 35 | 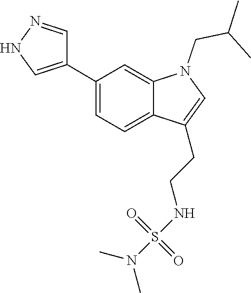 [SMILES: CC(C)Cn1cc(CCNS(=O)(=O)N(C)C)c2ccc(-c3cn[nH]c3)cc21] |
| 36 | 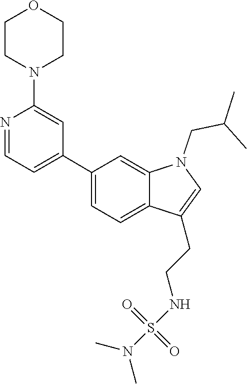 [SMILES: CC(C)Cn1cc(CCNS(=O)(=O)N(C)C)c2ccc(-c3ccnc(N4CCOCC4)c3)cc21] |
| 37 | 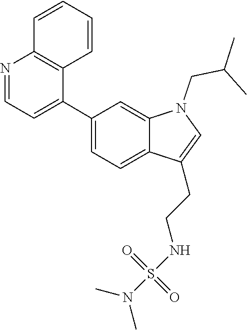 [SMILES: CC(C)Cn1cc(CCNS(=O)(=O)N(C)C)c2ccc(-c3ccnc4ccccc34)cc21] |
| 38 | 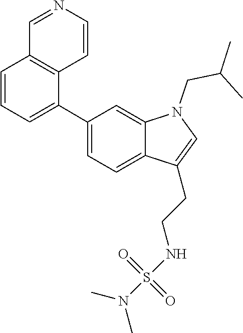 [SMILES: CC(C)Cn1cc(CCNS(=O)(=O)N(C)C)c2ccc(-c3cccc4cnccc34)cc21] |
| 39 | 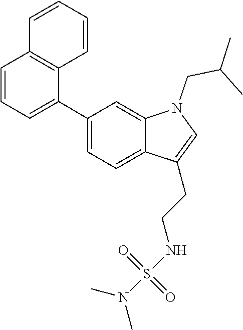 [SMILES: CC(C)Cn1cc(CCNS(=O)(=O)N(C)C)c2ccc(-c3cccc4ccccc34)cc21] |
| 40 | 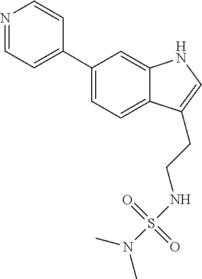 [SMILES: CN(C)S(=O)(=O)NCCc1c[nH]c2cc(-c3ccncc3)ccc12] |
| 41 | 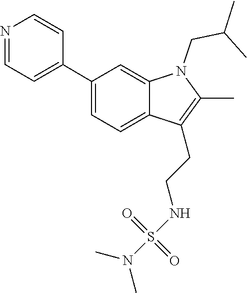 [SMILES: Cc1c(CCNS(=O)(=O)N(C)C)c2ccc(-c3ccncc3)cc2n1CC(C)C] |
| 42 | 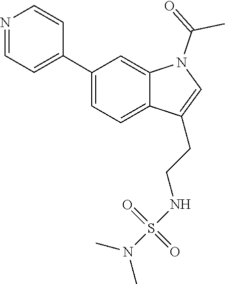 [SMILES: CC(=O)n1cc(CCNS(=O)(=O)N(C)C)c2ccc(-c3ccncc3)cc21] |
| 43 | 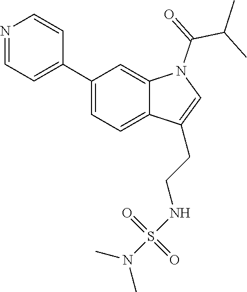 [SMILES: CC(C)C(=O)n1cc(CCNS(=O)(=O)N(C)C)c2ccc(-c3ccncc3)cc21] |
| 44 | 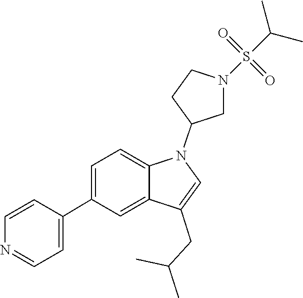 [SMILES: CC(C)Cc1cn(C2CCN(S(=O)(=O)C(C)C)C2)c2ccc(-c3ccncc3)cc12] |
| 45 | 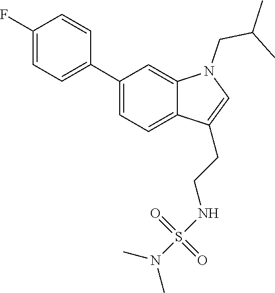 [SMILES: CC(C)Cn1cc(CCNS(=O)(=O)N(C)C)c2ccc(-c3ccc(F)cc3)cc21] |
| 46 | 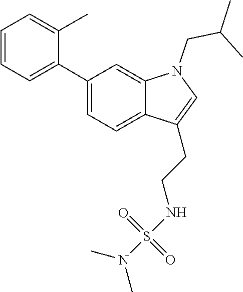 [SMILES: Cc1ccccc1-c1ccc2c(CCNS(=O)(=O)N(C)C)cn(CC(C)C)c2c1] |
| 47 | 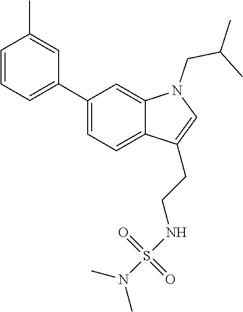 [SMILES: Cc1cccc(-c2ccc3c(CCNS(=O)(=O)N(C)C)cn(CC(C)C)c3c2)c1] |
| 48 | 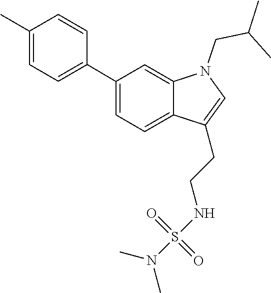 [SMILES: Cc1ccc(-c2ccc3c(CCNS(=O)(=O)N(C)C)cn(CC(C)C)c3c2)cc1] |
| 49 | 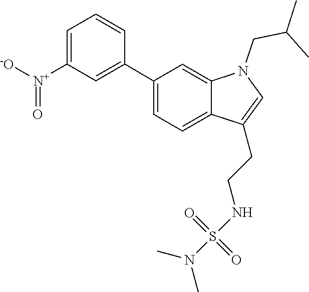 |
| 50 | 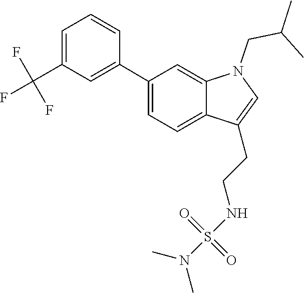 [SMILES: CC(C)Cn1cc(CCNS(=O)(=O)N(C)C)c2ccc(-c3cccc(C(F)(F)F)c3)cc21] |
| 51 | 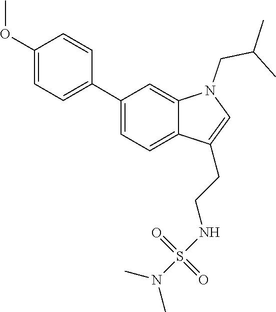 [SMILES: COc1ccc(-c2ccc3c(CCNS(=O)(=O)N(C)C)cn(CC(C)C)c3c2)cc1] |
| 52 | 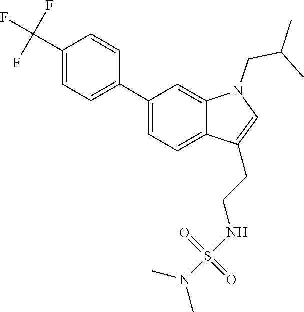 [SMILES: CC(C)Cn1cc(CCNS(=O)(=O)N(C)C)c2ccc(-c3ccc(C(F)(F)F)cc3)cc21] |
| 53 | 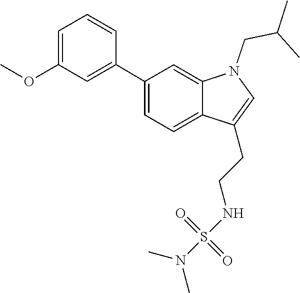 [SMILES: COc1cccc(-c2ccc3c(CCNS(=O)(=O)N(C)C)cn(CC(C)C)c3c2)c1] |
| 54 | 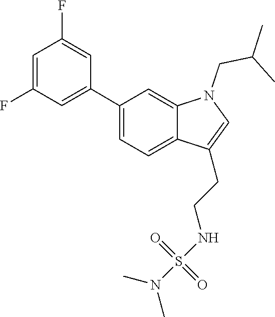 [SMILES: CC(C)Cn1cc(CCNS(=O)(=O)N(C)C)c2ccc(-c3cc(F)cc(F)c3)cc21] |
| 55 | 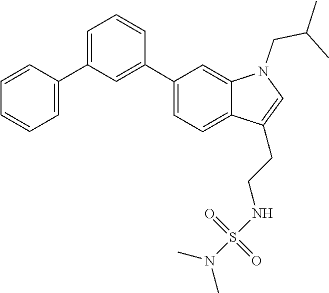 [SMILES: CC(C)Cn1cc(CCNS(=O)(=O)N(C)C)c2ccc(-c3cccc(-c4ccccc4)c3)cc21] |
| 56 | 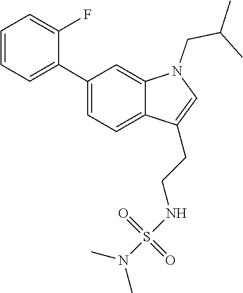 [SMILES: CC(C)Cn1cc(CCNS(=O)(=O)N(C)C)c2ccc(-c3ccccc3F)cc21] |
| 57 | 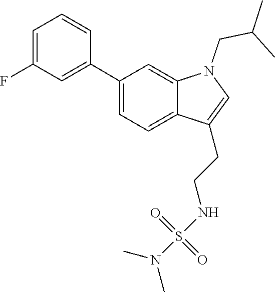 [SMILES: CC(C)Cn1cc(CCNS(=O)(=O)N(C)C)c2ccc(-c3cccc(F)c3)cc21] |
| 58 | 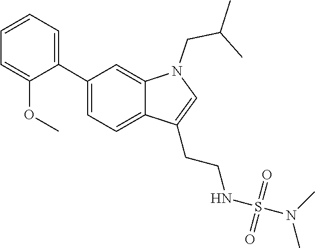 [SMILES: COc1ccccc1-c1ccc2c(CCNS(=O)(=O)N(C)C)cn(CC(C)C)c2c1] |
| 59 | 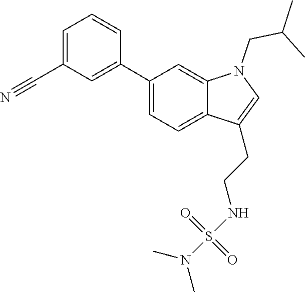 [SMILES: CC(C)Cn1cc(CCNS(=O)(=O)N(C)C)c2ccc(-c3cccc(C#N)c3)cc21] |
| 60 | 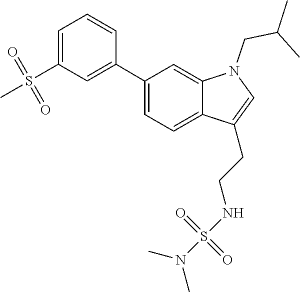 [SMILES: CC(C)Cn1cc(CCNS(=O)(=O)N(C)C)c2ccc(-c3cccc(S(C)(=O)=O)c3)cc21] |
| 61 | 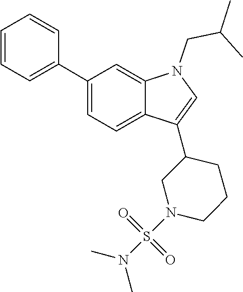 [SMILES: CC(C)Cn1cc(C2CCCN(S(=O)(=O)N(C)C)C2)c2ccc(-c3ccccc3)cc21] |
| 62 | 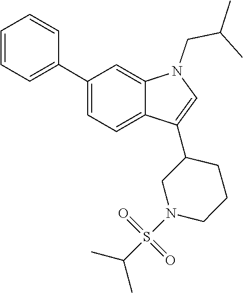 [SMILES: CC(C)Cn1cc(C2CCCN(S(=O)(=O)C(C)C)C2)c2ccc(-c3ccccc3)cc21] |
| 63 | 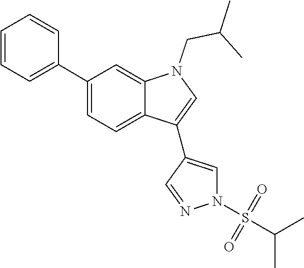 [SMILES: CC(C)Cn1cc(-c2cnn(S(=O)(=O)C(C)C)c2)c2ccc(-c3ccccc3)cc21] |
| 64 | 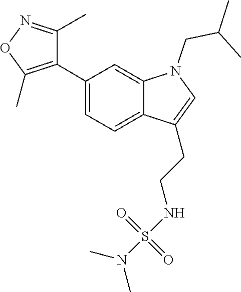 [SMILES: Cc1noc(C)c1-c1ccc2c(CCNS(=O)(=O)N(C)C)cn(CC(C)C)c2c1] |
| 65 | 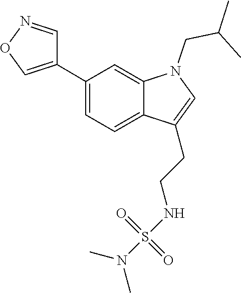 [SMILES: CC(C)Cn1cc(CCNS(=O)(=O)N(C)C)c2ccc(-c3cnoc3)cc21] |
| 66 | 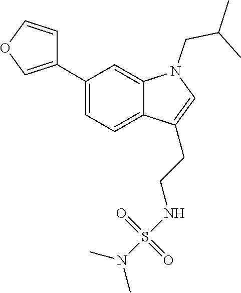 [SMILES: CC(C)Cn1cc(CCNS(=O)(=O)N(C)C)c2ccc(-c3ccoc3)cc21] |
| 67 | 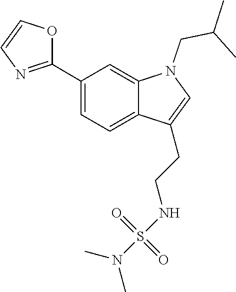 [SMILES: CC(C)Cn1cc(CCNS(=O)(=O)N(C)C)c2ccc(-c3ncco3)cc21] |
| 68 | 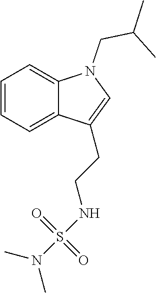 [SMILES: CC(C)Cn1cc(CCNS(=O)(=O)N(C)C)c2ccccc21] |
| 69 | 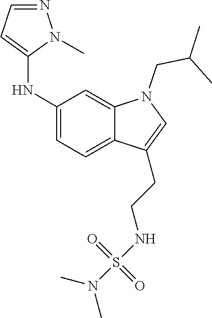 [SMILES: CC(C)Cn1cc(CCNS(=O)(=O)N(C)C)c2ccc(Nc3ccnn3C)cc21] |
| 70 | 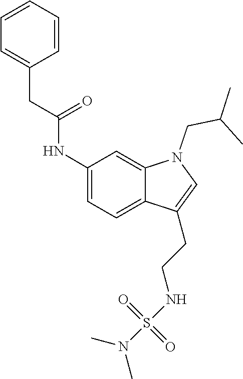 [SMILES: CC(C)Cn1cc(CCNS(=O)(=O)N(C)C)c2ccc(NC(=O)Cc3ccccc3)cc21] |
| 71 | 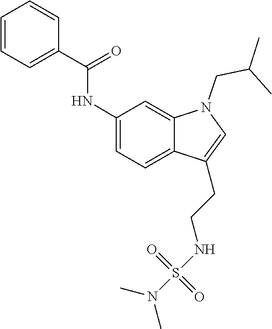 [SMILES: CC(C)Cn1cc(CCNS(=O)(=O)N(C)C)c2ccc(NC(=O)c3ccccc3)cc21] |
| 72 | 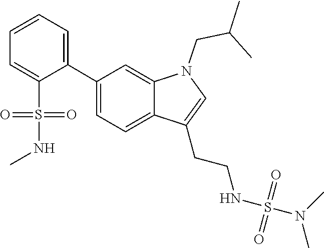 [SMILES: CNS(=O)(=O)c1ccccc1-c1ccc2c(CCNS(=O)(=O)N(C)C)cn(CC(C)C)c2c1] |
| 73 | 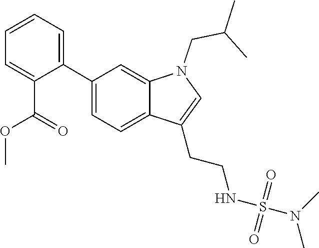 [SMILES: COC(=O)c1ccccc1-c1ccc2c(CCNS(=O)(=O)N(C)C)cn(CC(C)C)c2c1] |
| 74 | 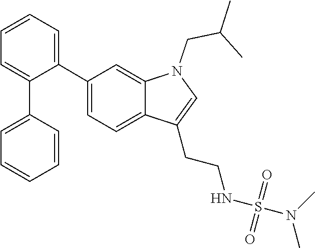 [SMILES: CC(C)Cn1cc(CCNS(=O)(=O)N(C)C)c2ccc(-c3ccccc3-c3ccccc3)cc21] |
| 75 | 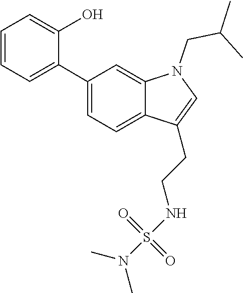 [SMILES: CC(C)Cn1cc(CCNS(=O)(=O)N(C)C)c2ccc(-c3ccccc3O)cc21] |
| 76 | 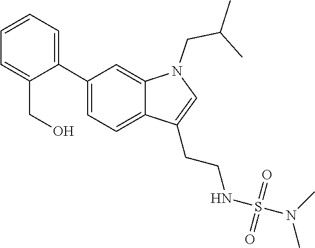 [SMILES: CC(C)Cn1cc(CCNS(=O)(=O)N(C)C)c2ccc(-c3ccccc3CO)cc21] |
| 77 | 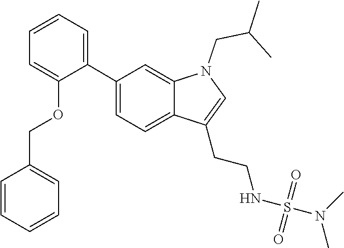 [SMILES: CC(C)Cn1cc(CCNS(=O)(=O)N(C)C)c2ccc(-c3ccccc3OCc3ccccc3)cc21] |
| 78 | 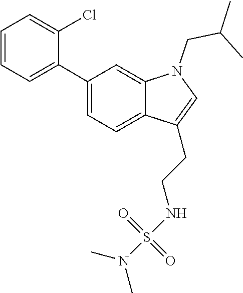 [SMILES: CC(C)Cn1cc(CCNS(=O)(=O)N(C)C)c2ccc(-c3ccccc3Cl)cc21] |
| 79 | 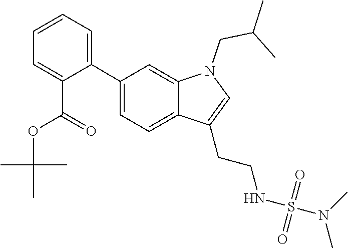 [SMILES: CC(C)Cn1cc(CCNS(=O)(=O)N(C)C)c2ccc(-c3ccccc3C(=O)OC(C)(C)C)cc21] |
| 80 | 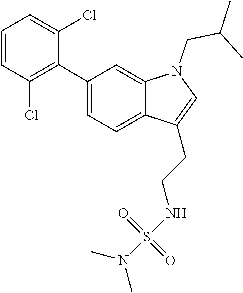 [SMILES: CC(C)Cn1cc(CCNS(=O)(=O)N(C)C)c2ccc(-c3c(Cl)cccc3Cl)cc21] |
| 81 | 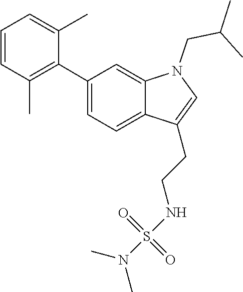 [SMILES: Cc1cccc(C)c1-c1ccc2c(CCNS(=O)(=O)N(C)C)cn(CC(C)C)c2c1] |
| 82 | 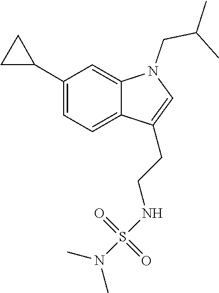 [SMILES: CC(C)Cn1cc(CCNS(=O)(=O)N(C)C)c2ccc(C3CC3)cc21] |
| 83 | 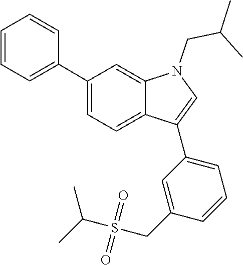 [SMILES: CC(C)Cn1cc(-c2cccc(CS(=O)(=O)C(C)C)c2)c2ccc(-c3ccccc3)cc21] |
| 84 | 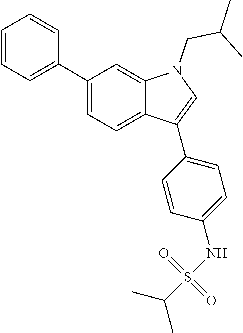 [SMILES: CC(C)Cn1cc(-c2ccc(NS(=O)(=O)C(C)C)cc2)c2ccc(-c3ccccc3)cc21] |
| 86 | 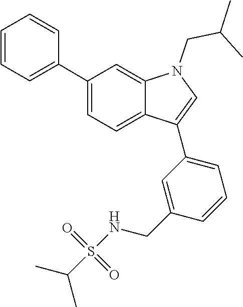 [SMILES: CC(C)Cn1cc(-c2cccc(CNS(=O)(=O)C(C)C)c2)c2ccc(-c3ccccc3)cc21] |
| 87 | 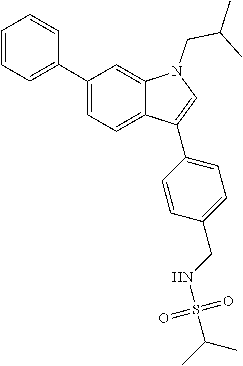 [SMILES: CC(C)Cn1cc(-c2ccc(CNS(=O)(=O)C(C)C)cc2)c2ccc(-c3ccccc3)cc21] |
| 88 | 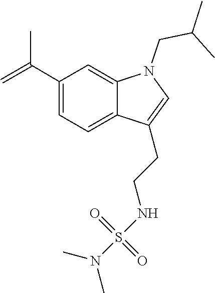 [SMILES: C=C(C)c1ccc2c(CCNS(=O)(=O)N(C)C)cn(CC(C)C)c2c1] |
| 91 | 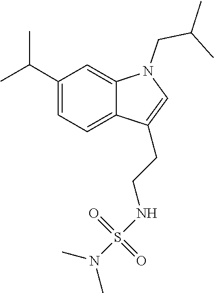 [SMILES: CC(C)Cn1cc(CCNS(=O)(=O)N(C)C)c2ccc(C(C)C)cc21] |
| 92 | 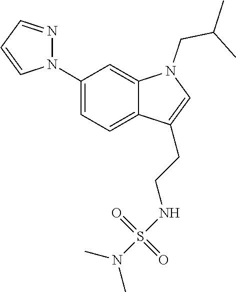 [SMILES: CC(C)Cn1cc(CCNS(=O)(=O)N(C)C)c2ccc(-n3cccn3)cc21] |
| 93 | 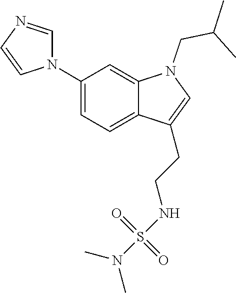 [SMILES: CC(C)Cn1cc(CCNS(=O)(=O)N(C)C)c2ccc(-n3ccnc3)cc21] |
| 95 | 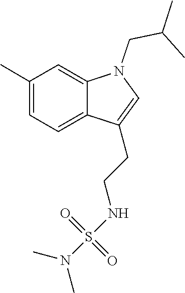 [SMILES: Cc1ccc2c(CCNS(=O)(=O)N(C)C)cn(CC(C)C)c2c1] |
| 96 | 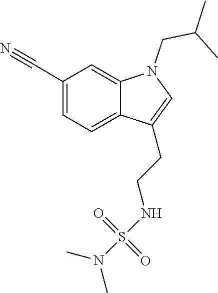 [SMILES: CC(C)Cn1cc(CCNS(=O)(=O)N(C)C)c2ccc(C#N)cc21] |
| 97 | 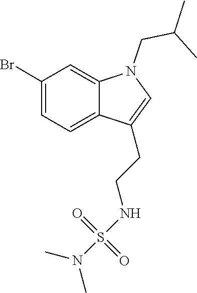 [SMILES: CC(C)Cn1cc(CCNS(=O)(=O)N(C)C)c2ccc(Br)cc21] |
| 98 | 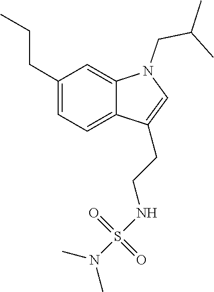 [SMILES: CCCc1ccc2c(CCNS(=O)(=O)N(C)C)cn(CC(C)C)c2c1] |
| 99 | 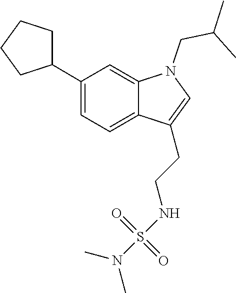 [SMILES: CC(C)Cn1cc(CCNS(=O)(=O)N(C)C)c2ccc(C3CCCC3)cc21] |
| 100 | 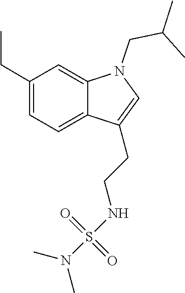 [SMILES: CCc1ccc2c(CCNS(=O)(=O)N(C)C)cn(CC(C)C)c2c1] |
| 101 | 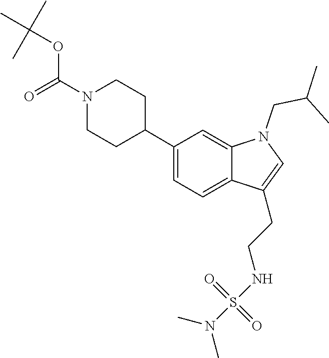 [SMILES: CC(C)Cn1cc(CCNS(=O)(=O)N(C)C)c2ccc(C3CCN(C(=O)OC(C)(C)C)CC3)cc21] |
| 102 | 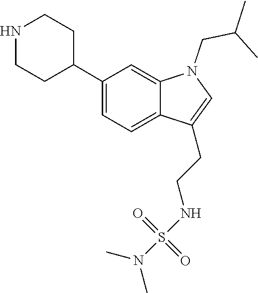 [SMILES: CC(C)Cn1cc(CCNS(=O)(=O)N(C)C)c2ccc(C3CCNCC3)cc21] |
| 103 | 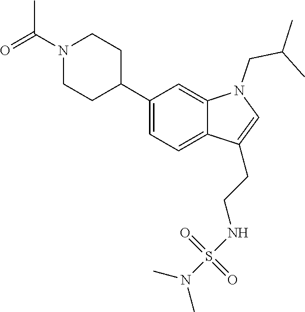 [SMILES: CC(=O)N1CCC(c2ccc3c(CCNS(=O)(=O)N(C)C)cn(CC(C)C)c3c2)CC1] |
| 105 | 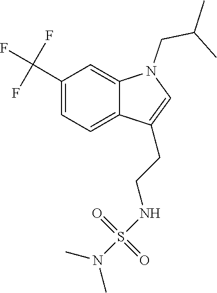 [SMILES: CC(C)Cn1cc(CCNS(=O)(=O)N(C)C)c2ccc(C(F)(F)F)cc21] |
| 106 | 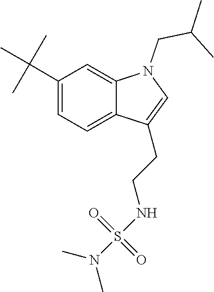 [SMILES: CC(C)Cn1cc(CCNS(=O)(=O)N(C)C)c2ccc(C(C)(C)C)cc21] |
| 107 | 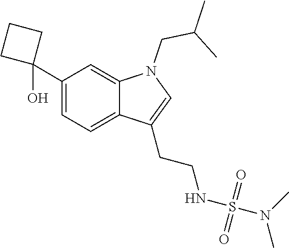 [SMILES: CC(C)Cn1cc(CCNS(=O)(=O)N(C)C)c2ccc(C3(O)CCC3)cc21] |
| 108 | 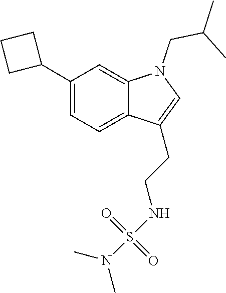 [SMILES: CC(C)Cn1cc(CCNS(=O)(=O)N(C)C)c2ccc(C3CCC3)cc21] |
| 109 | 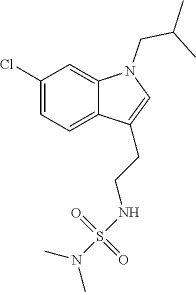 [SMILES: CC(C)Cn1cc(CCNS(=O)(=O)N(C)C)c2ccc(Cl)cc21] |
| 110 | 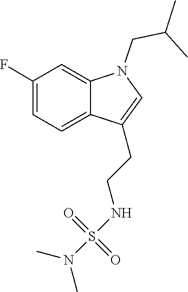 [SMILES: CC(C)Cn1cc(CCNS(=O)(=O)N(C)C)c2ccc(F)cc21] |
| 111 | 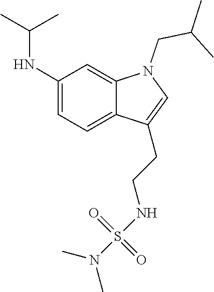 [SMILES: CC(C)Cn1cc(CCNS(=O)(=O)N(C)C)c2ccc(NC(C)C)cc21] |
| 112 | 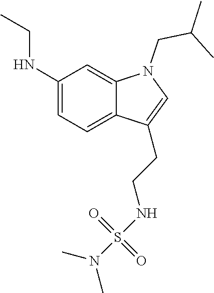 [SMILES: CCNc1ccc2c(CCNS(=O)(=O)N(C)C)cn(CC(C)C)c2c1] |
| 113 | 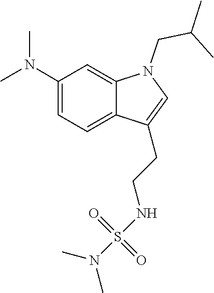 [SMILES: CC(C)Cn1cc(CCNS(=O)(=O)N(C)C)c2ccc(N(C)C)cc21] |
| 114 | 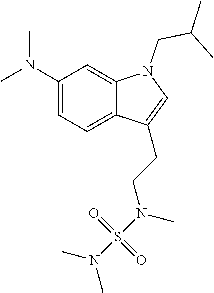 [SMILES: CC(C)Cn1cc(CCN(C)S(=O)(=O)N(C)C)c2ccc(N(C)C)cc21] |
| 115 | 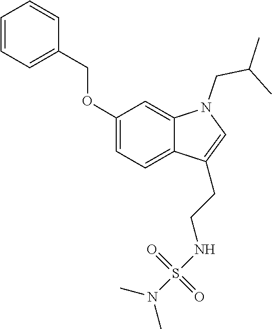 [SMILES: CC(C)Cn1cc(CCNS(=O)(=O)N(C)C)c2ccc(OCc3ccccc3)cc21] |
| 116 | 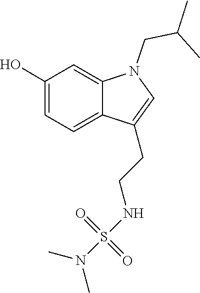 [SMILES: CC(C)Cn1cc(CCNS(=O)(=O)N(C)C)c2ccc(O)cc21] |
| 117 | 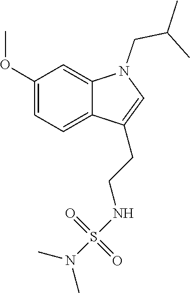 [SMILES: COc1ccc2c(CCNS(=O)(=O)N(C)C)cn(CC(C)C)c2c1] |
| 118 | 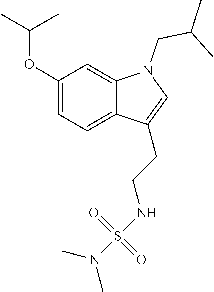 [SMILES: CC(C)Cn1cc(CCNS(=O)(=O)N(C)C)c2ccc(OC(C)C)cc21] |
| 119 | 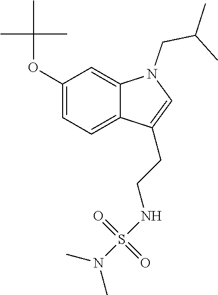 [SMILES: CC(C)Cn1cc(CCNS(=O)(=O)N(C)C)c2ccc(OC(C)(C)C)cc21] |
| 120 | 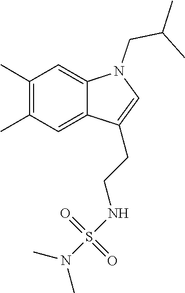 [SMILES: Cc1cc2c(CCNS(=O)(=O)N(C)C)cn(CC(C)C)c2cc1C] |
| 121 | 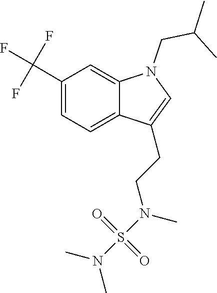 [SMILES: CC(C)Cn1cc(CCN(C)S(=O)(=O)N(C)C)c2ccc(C(F)(F)F)cc21] |
| 122 | 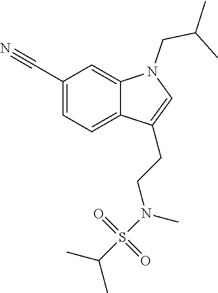 [SMILES: CC(C)Cn1cc(CCN(C)S(=O)(=O)C(C)C)c2ccc(C#N)cc21] |
| 123 | 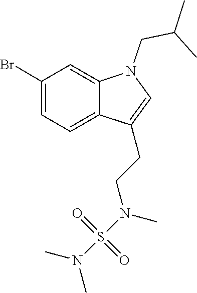 [SMILES: CC(C)Cn1cc(CCN(C)S(=O)(=O)N(C)C)c2ccc(Br)cc21] |
| 124 | 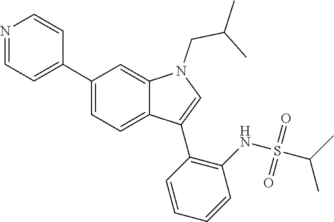 [SMILES: CC(C)Cn1cc(-c2ccccc2NS(=O)(=O)C(C)C)c2ccc(-c3ccncc3)cc21] |
| 125 | 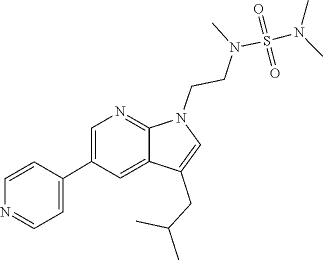 [SMILES: CC(C)Cc1cn(CCN(C)S(=O)(=O)N(C)C)c2ncc(-c3ccncc3)cc12] |
| 126 | 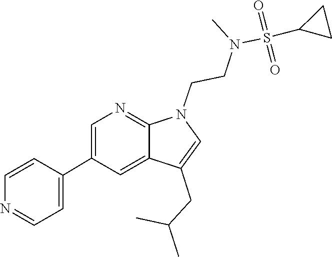 [SMILES: CC(C)Cc1cn(CCN(C)S(=O)(=O)C2CC2)c2ncc(-c3ccncc3)cc12] |
| 127 | 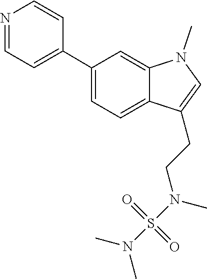 [SMILES: CN(C)S(=O)(=O)N(C)CCc1cn(C)c2cc(-c3ccncc3)ccc12] |
| 128 | 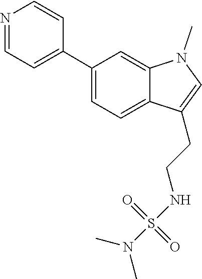 [SMILES: CN(C)S(=O)(=O)NCCc1cn(C)c2cc(-c3ccncc3)ccc12] |
| 129 | 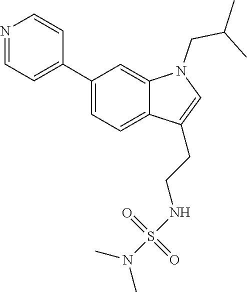 [SMILES: CC(C)Cn1cc(CCNS(=O)(=O)N(C)C)c2ccc(-c3ccncc3)cc21] |
| 130 | 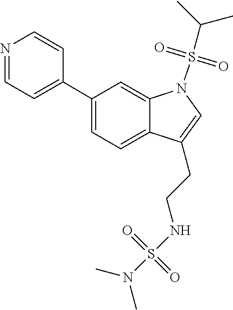 [SMILES: CC(C)S(=O)(=O)n1cc(CCNS(=O)(=O)N(C)C)c2ccc(-c3ccncc3)cc21] |
| 131 | 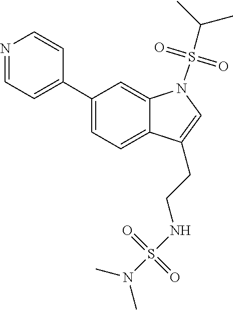 [SMILES: CC(C)S(=O)(=O)n1cc(CCNS(=O)(=O)N(C)C)c2ccc(-c3ccncc3)cc21] |
| 132 | 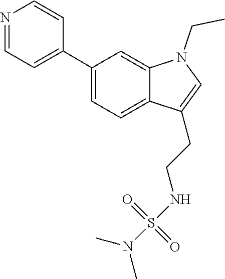 [SMILES: CCn1cc(CCNS(=O)(=O)N(C)C)c2ccc(-c3ccncc3)cc21] |
| 133 | 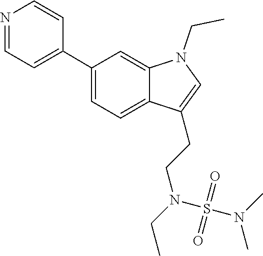 [SMILES: CCN(CCc1cn(CC)c2cc(-c3ccncc3)ccc12)S(=O)(=O)N(C)C] |
| 134 | 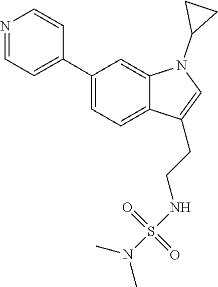 [SMILES: CN(C)S(=O)(=O)NCCc1cn(C2CC2)c2cc(-c3ccncc3)ccc12] |
| 135 | 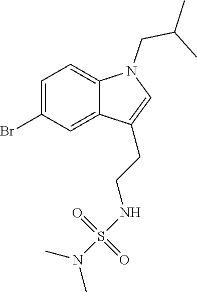 [SMILES: CC(C)Cn1cc(CCNS(=O)(=O)N(C)C)c2cc(Br)ccc21] |
| 136 | 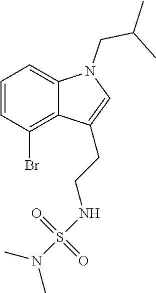 [SMILES: CC(C)Cn1cc(CCNS(=O)(=O)N(C)C)c2c(Br)cccc21] |
| 137 | 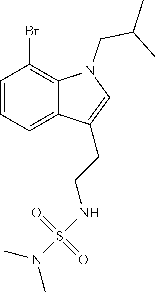 [SMILES: CC(C)Cn1cc(CCNS(=O)(=O)N(C)C)c2cccc(Br)c21] |
| 140 | 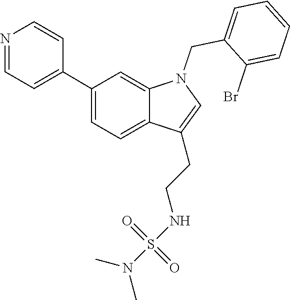 [SMILES: CN(C)S(=O)(=O)NCCc1cn(Cc2ccccc2Br)c2cc(-c3ccncc3)ccc12] |
| 141 | 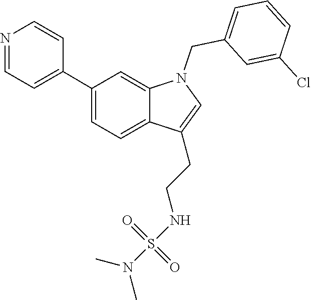 [SMILES: CN(C)S(=O)(=O)NCCc1cn(Cc2cccc(Cl)c2)c2cc(-c3ccncc3)ccc12] |
| 142 | 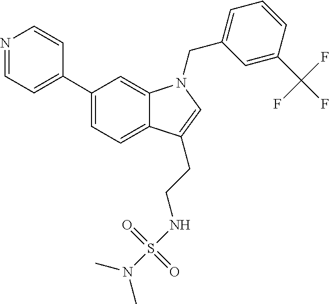 [SMILES: CN(C)S(=O)(=O)NCCc1cn(Cc2cccc(C(F)(F)F)c2)c2cc(-c3ccncc3)ccc12] |
| 143 | 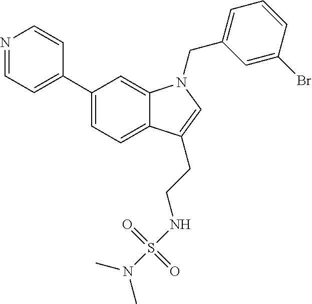 [SMILES: CN(C)S(=O)(=O)NCCc1cn(Cc2cccc(Br)c2)c2cc(-c3ccncc3)ccc12] |
| 144 | 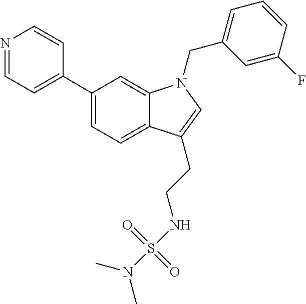 [SMILES: CN(C)S(=O)(=O)NCCc1cn(Cc2cccc(F)c2)c2cc(-c3ccncc3)ccc12] |
| 145 | 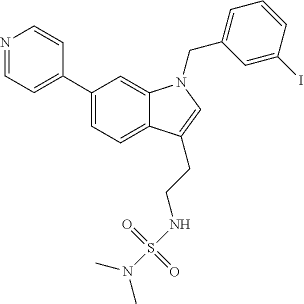 [SMILES: CN(C)S(=O)(=O)NCCc1cn(Cc2cccc(I)c2)c2cc(-c3ccncc3)ccc12] |
| 146 | 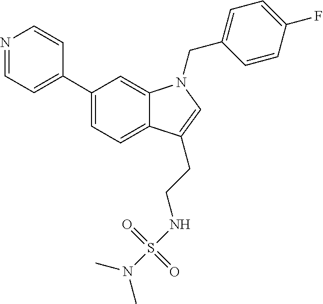 [SMILES: CN(C)S(=O)(=O)NCCc1cn(Cc2ccc(F)cc2)c2cc(-c3ccncc3)ccc12] |
| 147 | 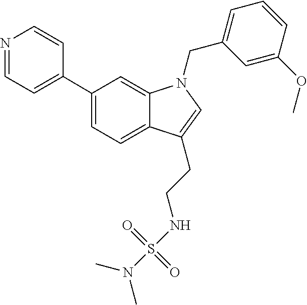 [SMILES: COc1cccc(Cn2cc(CCNS(=O)(=O)N(C)C)c3ccc(-c4ccncc4)cc32)c1] |
| 148 | 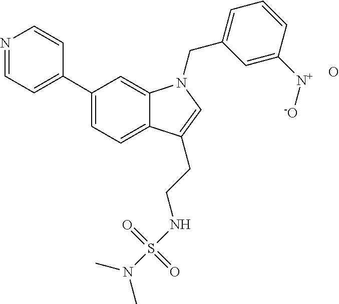 |
| 149 | 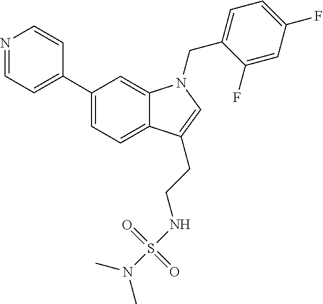 [SMILES: CN(C)S(=O)(=O)NCCc1cn(Cc2ccc(F)cc2F)c2cc(-c3ccncc3)ccc12] |
| 150 | 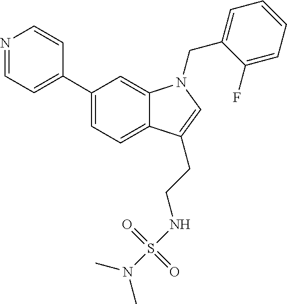 [SMILES: CN(C)S(=O)(=O)NCCc1cn(Cc2ccccc2F)c2cc(-c3ccncc3)ccc12] |
| 151 | 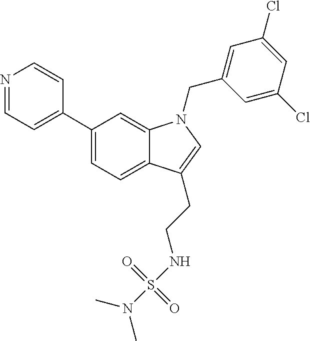 [SMILES: CN(C)S(=O)(=O)NCCc1cn(Cc2cc(Cl)cc(Cl)c2)c2cc(-c3ccncc3)ccc12] |
| 152 | 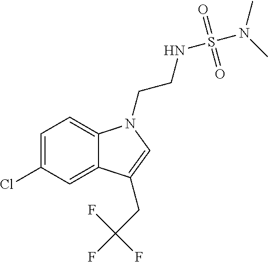 [SMILES: CN(C)S(=O)(=O)NCCn1cc(CC(F)(F)F)c2cc(Cl)ccc21] |
| 153 | 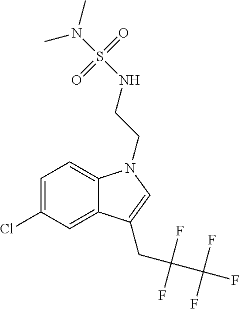 [SMILES: CN(C)S(=O)(=O)NCCn1cc(CC(F)(F)C(F)(F)F)c2cc(Cl)ccc21] |
| 154 | 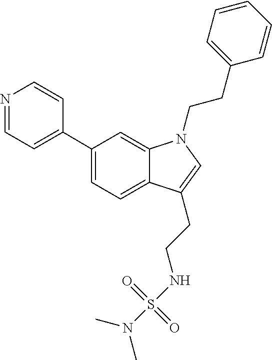 [SMILES: CN(C)S(=O)(=O)NCCc1cn(CCc2ccccc2)c2cc(-c3ccncc3)ccc12] |
| 155 | 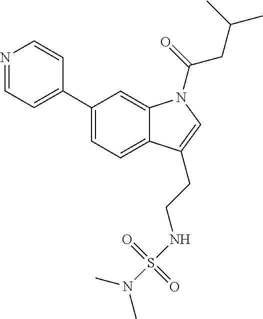 [SMILES: CC(C)CC(=O)n1cc(CCNS(=O)(=O)N(C)C)c2ccc(-c3ccncc3)cc21] |
| 156 | 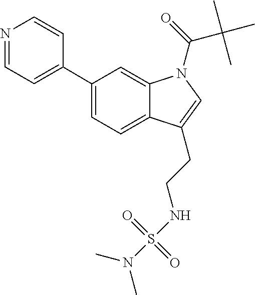 [SMILES: CN(C)S(=O)(=O)NCCc1cn(C(=O)C(C)(C)C)c2cc(-c3ccncc3)ccc12] |
| 157 | 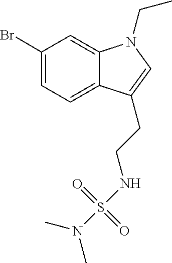 [SMILES: CCn1cc(CCNS(=O)(=O)N(C)C)c2ccc(Br)cc21] |
| 158 | 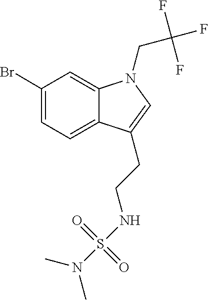 [SMILES: CN(C)S(=O)(=O)NCCc1cn(CC(F)(F)F)c2cc(Br)ccc12] |
| 159 | 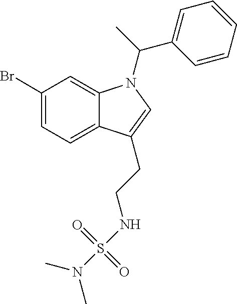 [SMILES: CC(c1ccccc1)n1cc(CCNS(=O)(=O)N(C)C)c2ccc(Br)cc21] |
| 160 | 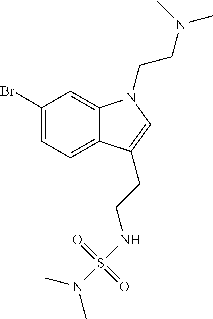 [SMILES: CN(C)CCn1cc(CCNS(=O)(=O)N(C)C)c2ccc(Br)cc21] |
| 161 | 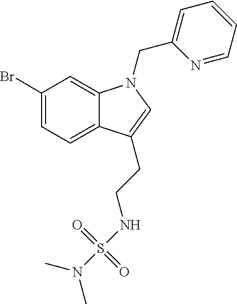 [SMILES: CN(C)S(=O)(=O)NCCc1cn(Cc2ccccn2)c2cc(Br)ccc12] |
| 162 | 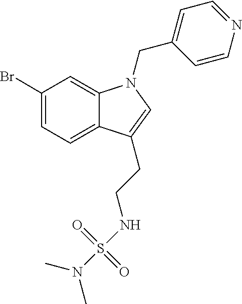 [SMILES: CN(C)S(=O)(=O)NCCc1cn(Cc2ccncc2)c2cc(Br)ccc12] |
| 163 | 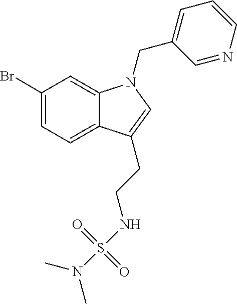 [SMILES: CN(C)S(=O)(=O)NCCc1cn(Cc2cccnc2)c2cc(Br)ccc12] |
| 164 | 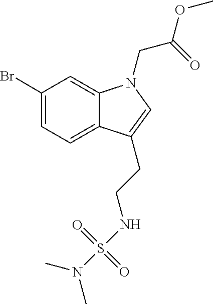 [SMILES: COC(=O)Cn1cc(CCNS(=O)(=O)N(C)C)c2ccc(Br)cc21] |
| 165 | 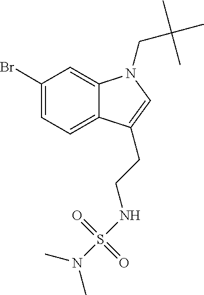 [SMILES: CN(C)S(=O)(=O)NCCc1cn(CC(C)(C)C)c2cc(Br)ccc12] |
| 166 | 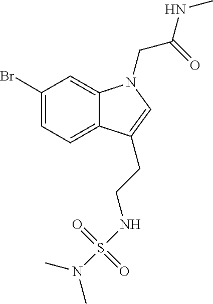 [SMILES: CNC(=O)Cn1cc(CCNS(=O)(=O)N(C)C)c2ccc(Br)cc21] |
| 167 | 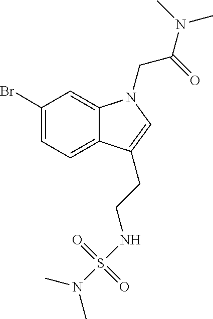 [SMILES: CN(C)C(=O)Cn1cc(CCNS(=O)(=O)N(C)C)c2ccc(Br)cc21] |
| 168 | 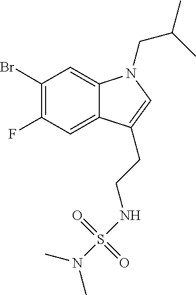 [SMILES: CC(C)Cn1cc(CCNS(=O)(=O)N(C)C)c2cc(F)c(Br)cc21] |
| 169 | 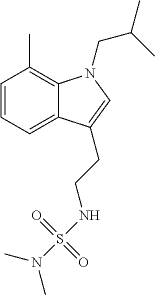 [SMILES: Cc1cccc2c(CCNS(=O)(=O)N(C)C)cn(CC(C)C)c12] |
| 170 | 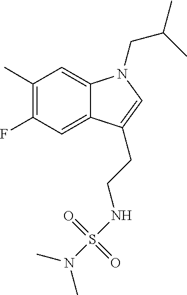 [SMILES: Cc1cc2c(cc1F)c(CCNS(=O)(=O)N(C)C)cn2CC(C)C] |
| 171 | 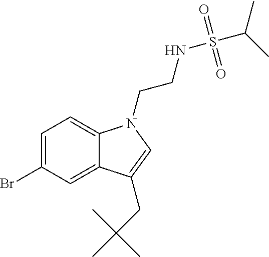 [SMILES: CC(C)S(=O)(=O)NCCn1cc(CC(C)(C)C)c2cc(Br)ccc21] |
| 172 | 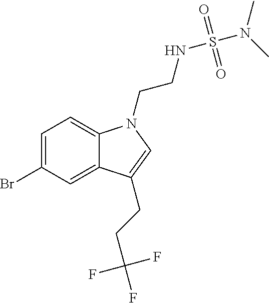 [SMILES: CN(C)S(=O)(=O)NCCn1cc(CCC(F)(F)F)c2cc(Br)ccc21] |
| 173 | 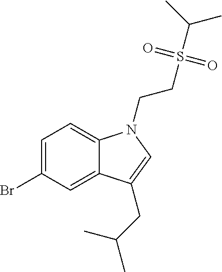 [SMILES: CC(C)Cc1cn(CCS(=O)(=O)C(C)C)c2ccc(Br)cc12] |
| 174 | 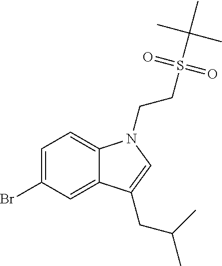 [SMILES: CC(C)Cc1cn(CCS(=O)(=O)C(C)(C)C)c2ccc(Br)cc12] |
| 175 | 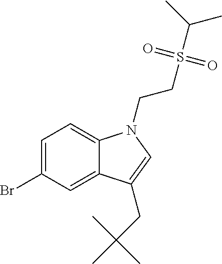 [SMILES: CC(C)S(=O)(=O)CCn1cc(CC(C)(C)C)c2cc(Br)ccc21] |
| 176 | 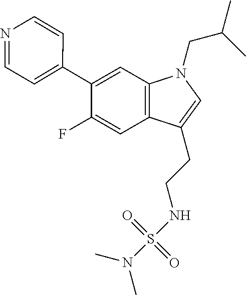 [SMILES: CC(C)Cn1cc(CCNS(=O)(=O)N(C)C)c2cc(F)c(-c3ccncc3)cc21] |
| 177 | 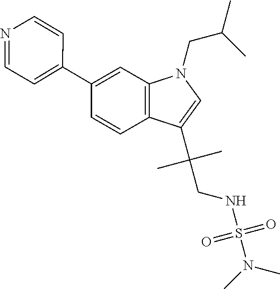 [SMILES: CC(C)Cn1cc(C(C)(C)CNS(=O)(=O)N(C)C)c2ccc(-c3ccncc3)cc21] |
| 178 | 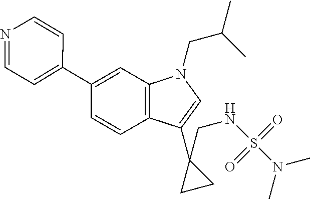 [SMILES: CC(C)Cn1cc(C2(CNS(=O)(=O)N(C)C)CC2)c2ccc(-c3ccncc3)cc21] |
| 179 | 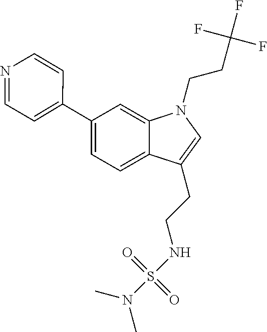 [SMILES: CN(C)S(=O)(=O)NCCc1cn(CCC(F)(F)F)c2cc(-c3ccncc3)ccc12] |
| 180 | 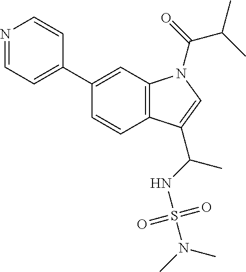 [SMILES: CC(C)C(=O)n1cc(C(C)NS(=O)(=O)N(C)C)c2ccc(-c3ccncc3)cc21] |
| 181 | 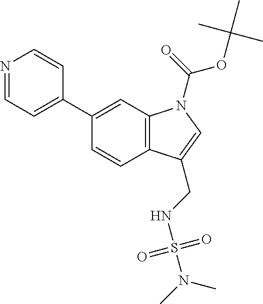 [SMILES: CN(C)S(=O)(=O)NCc1cn(C(=O)OC(C)(C)C)c2cc(-c3ccncc3)ccc12] |
| 182 | 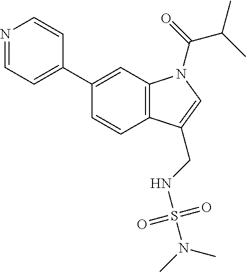 [SMILES: CC(C)C(=O)n1cc(CNS(=O)(=O)N(C)C)c2ccc(-c3ccncc3)cc21] |
| 183 | 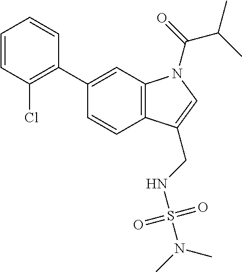 [SMILES: CC(C)C(=O)n1cc(CNS(=O)(=O)N(C)C)c2ccc(-c3ccccc3Cl)cc21] |
| 184 | 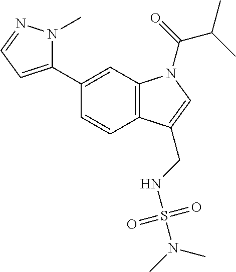 [SMILES: CC(C)C(=O)n1cc(CNS(=O)(=O)N(C)C)c2ccc(-c3ccnn3C)cc21] |
| 185 | 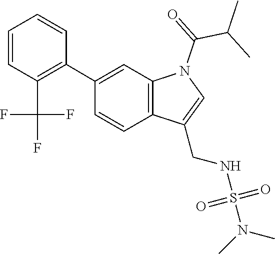 [SMILES: CC(C)C(=O)n1cc(CNS(=O)(=O)N(C)C)c2ccc(-c3ccccc3C(F)(F)F)cc21] |
| 186 | 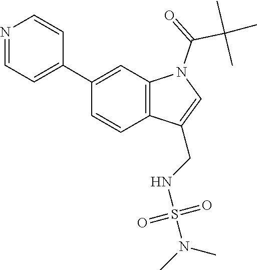 [SMILES: CN(C)S(=O)(=O)NCc1cn(C(=O)C(C)(C)C)c2cc(-c3ccncc3)ccc12] |
| 187 | 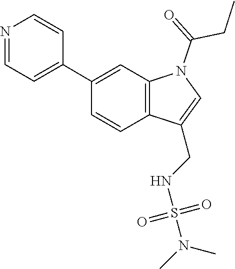 [SMILES: CCC(=O)n1cc(CNS(=O)(=O)N(C)C)c2ccc(-c3ccncc3)cc21] |
| 188 | 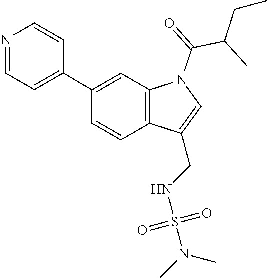 [SMILES: CCC(C)C(=O)n1cc(CNS(=O)(=O)N(C)C)c2ccc(-c3ccncc3)cc21] |
| 189 | 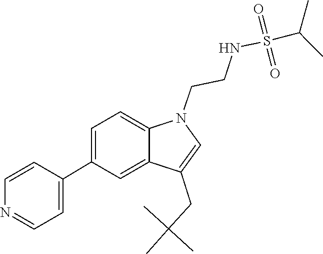 [SMILES: CC(C)S(=O)(=O)NCCn1cc(CC(C)(C)C)c2cc(-c3ccncc3)ccc21] |
| 190 | 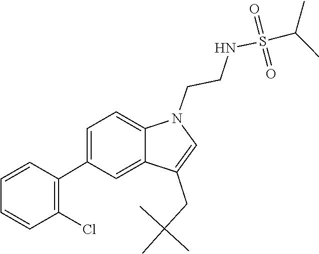 [SMILES: CC(C)S(=O)(=O)NCCn1cc(CC(C)(C)C)c2cc(-c3ccccc3Cl)ccc21] |
| 191 | 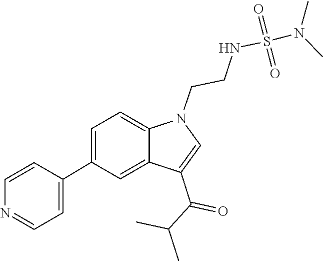 [SMILES: CC(C)C(=O)c1cn(CCNS(=O)(=O)N(C)C)c2ccc(-c3ccncc3)cc12] |
| 192 | 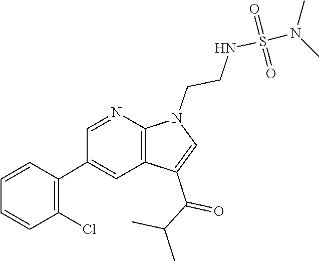 [SMILES: CC(C)C(=O)c1cn(CCNS(=O)(=O)N(C)C)c2ncc(-c3ccccc3Cl)cc12] |
| 194 | 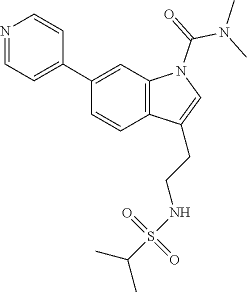 [SMILES: CC(C)S(=O)(=O)NCCc1cn(C(=O)N(C)C)c2cc(-c3ccncc3)ccc12] |
| 195 | 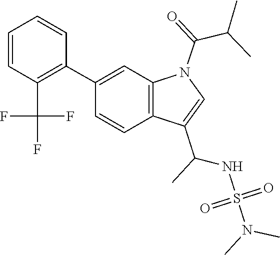 [SMILES: CC(C)C(=O)n1cc(C(C)NS(=O)(=O)N(C)C)c2ccc(-c3ccccc3C(F)(F)F)cc21] |
| 196 | 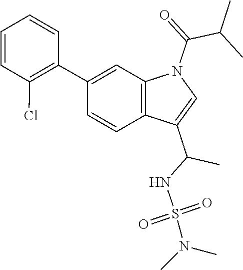 |
| 197 | 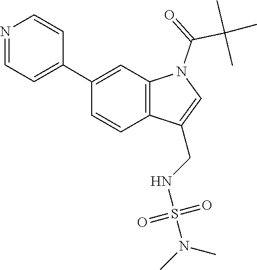 [SMILES: CN(C)S(=O)(=O)NCc1cn(C(=O)C(C)(C)C)c2cc(-c3ccncc3)ccc12] |
| 199 | 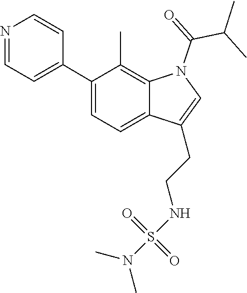 [SMILES: Cc1c(-c2ccncc2)ccc2c(CCNS(=O)(=O)N(C)C)cn(C(=O)C(C)C)c12] |
| 200 | 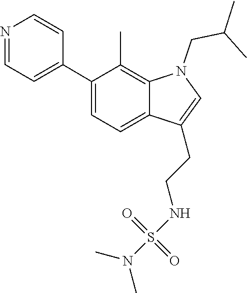 [SMILES: Cc1c(-c2ccncc2)ccc2c(CCNS(=O)(=O)N(C)C)cn(CC(C)C)c12] |
| 202 | 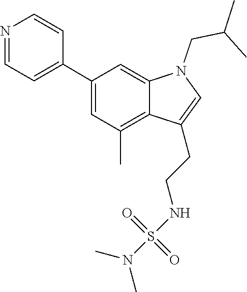 [SMILES: Cc1cc(-c2ccncc2)cc2c1c(CCNS(=O)(=O)N(C)C)cn2CC(C)C] |
| 203 | 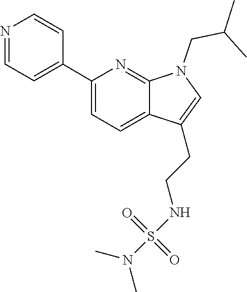 [SMILES: CC(C)Cn1cc(CCNS(=O)(=O)N(C)C)c2ccc(-c3ccncc3)nc21] |
| 204 | 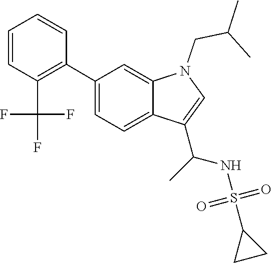 [SMILES: CC(C)Cn1cc(C(C)NS(=O)(=O)C2CC2)c2ccc(-c3ccccc3C(F)(F)F)cc21] |
| 205 | 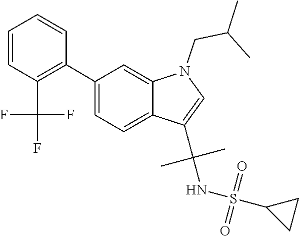 [SMILES: CC(C)Cn1cc(C(C)(C)NS(=O)(=O)C2CC2)c2ccc(-c3ccccc3C(F)(F)F)cc21] |
| 206 | 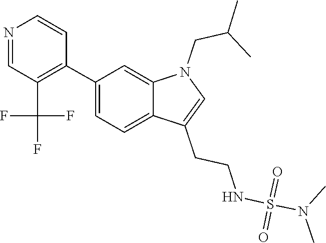 [SMILES: CC(C)Cn1cc(CCNS(=O)(=O)N(C)C)c2ccc(-c3ccncc3C(F)(F)F)cc21] |
| 207 | 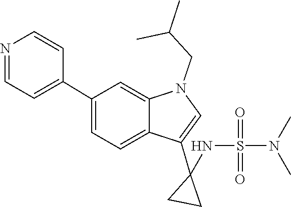 [SMILES: CC(C)Cn1cc(C2(NS(=O)(=O)N(C)C)CC2)c2ccc(-c3ccncc3)cc21] |
| 208 | 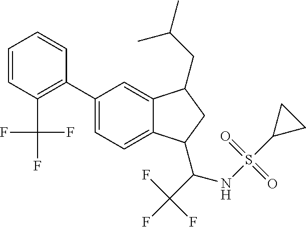 [SMILES: CC(C)CC1CC(C(NS(=O)(=O)C2CC2)C(F)(F)F)c2ccc(-c3ccccc3C(F)(F)F)cc21] |
| 209 | 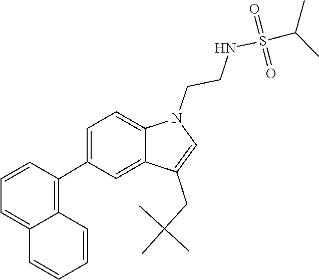 [SMILES: CC(C)S(=O)(=O)NCCn1cc(CC(C)(C)C)c2cc(-c3cccc4ccccc34)ccc21] |
| 210 | 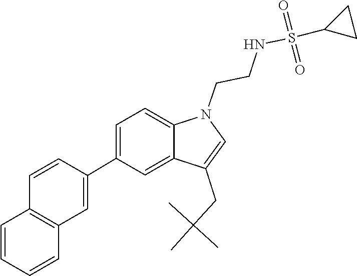 [SMILES: CC(C)(C)Cc1cn(CCNS(=O)(=O)C2CC2)c2ccc(-c3ccc4ccccc4c3)cc12] |
| 211 | 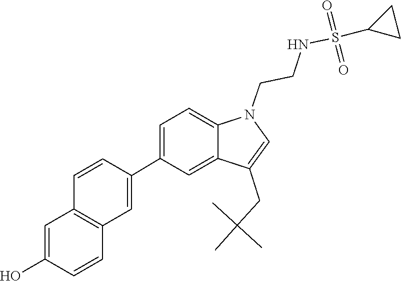 [SMILES: CC(C)(C)Cc1cn(CCNS(=O)(=O)C2CC2)c2ccc(-c3ccc4cc(O)ccc4c3)cc12] |
| 212 | 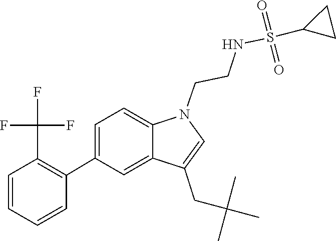 [SMILES: CC(C)(C)Cc1cn(CCNS(=O)(=O)C2CC2)c2ccc(-c3ccccc3C(F)(F)F)cc12] |
| 213 | 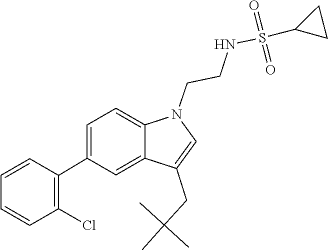 [SMILES: CC(C)(C)Cc1cn(CCNS(=O)(=O)C2CC2)c2ccc(-c3ccccc3Cl)cc12] |
| 214 | 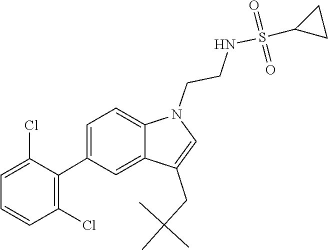 [SMILES: CC(C)(C)Cc1cn(CCNS(=O)(=O)C2CC2)c2ccc(-c3c(Cl)cccc3Cl)cc12] |
| 215 | 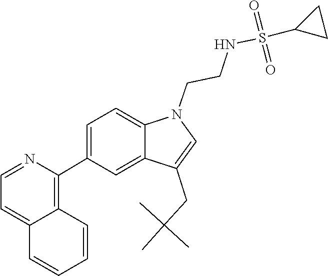 [SMILES: CC(C)(C)Cc1cn(CCNS(=O)(=O)C2CC2)c2ccc(-c3nccc4ccccc34)cc12] |
| 216 | 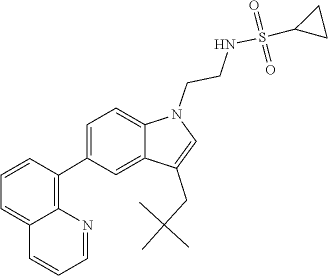 [SMILES: CC(C)(C)Cc1cn(CCNS(=O)(=O)C2CC2)c2ccc(-c3cccc4cccnc34)cc12] |
| 217 | 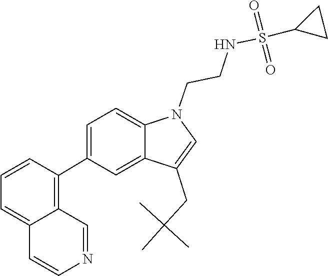 [SMILES: CC(C)(C)Cc1cn(CCNS(=O)(=O)C2CC2)c2ccc(-c3cccc4ccncc34)cc12] |
| 218 | 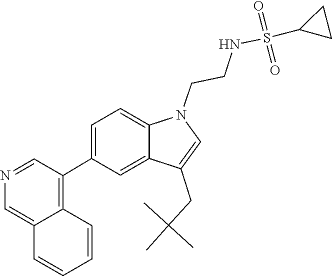 [SMILES: CC(C)(C)Cc1cn(CCNS(=O)(=O)C2CC2)c2ccc(-c3cncc4ccccc34)cc12] |
| 219 | 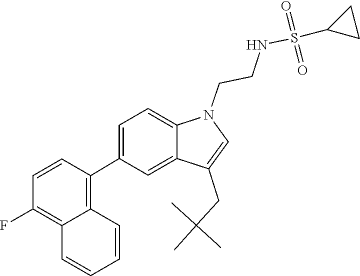 [SMILES: CC(C)(C)Cc1cn(CCNS(=O)(=O)C2CC2)c2ccc(-c3ccc(F)c4ccccc34)cc12] |
| 220 | 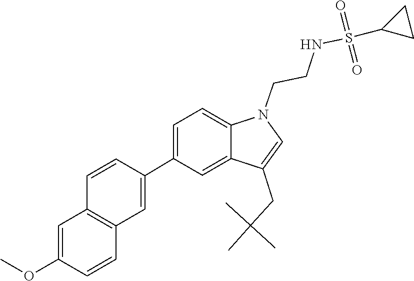 [SMILES: COc1ccc2cc(-c3ccc4c(c3)c(CC(C)(C)C)cn4CCNS(=O)(=O)C3CC3)ccc2c1] |
| 221 | 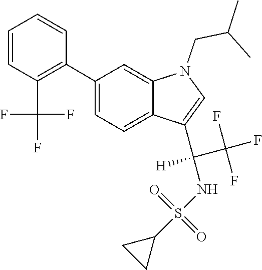 [SMILES: CC(C)Cn1cc([C@H](NS(=O)(=O)C2CC2)C(F)(F)F)c2ccc(-c3ccccc3C(F)(F)F)cc21] |
| 222 | 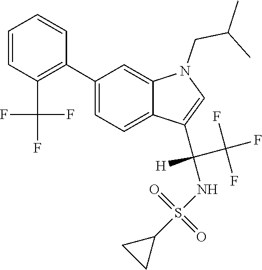 [SMILES: CC(C)Cn1cc([C@@H](NS(=O)(=O)C2CC2)C(F)(F)F)c2ccc(-c3ccccc3C(F)(F)F)cc21] |
| 223 | 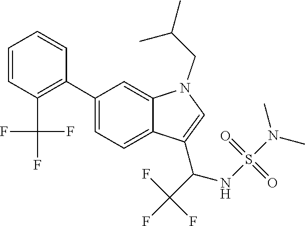 [SMILES: CC(C)Cn1cc(C(NS(=O)(=O)N(C)C)C(F)(F)F)c2ccc(-c3ccccc3C(F)(F)F)cc21] |
| 224 | 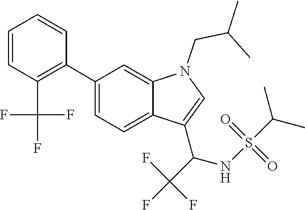 [SMILES: CC(C)Cn1cc(C(NS(=O)(=O)C(C)C)C(F)(F)F)c2ccc(-c3ccccc3C(F)(F)F)cc21] |
| 225 | 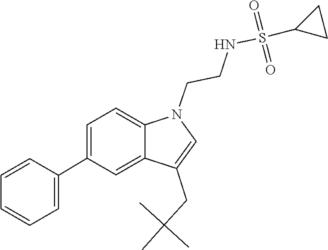 [SMILES: CC(C)(C)Cc1cn(CCNS(=O)(=O)C2CC2)c2ccc(-c3ccccc3)cc12] |
| 226 | 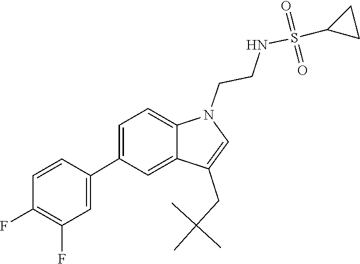 [SMILES: CC(C)(C)Cc1cn(CCNS(=O)(=O)C2CC2)c2ccc(-c3ccc(F)c(F)c3)cc12] |
| 227 | 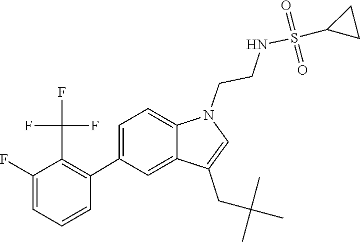 [SMILES: CC(C)(C)Cc1cn(CCNS(=O)(=O)C2CC2)c2ccc(-c3cccc(F)c3C(F)(F)F)cc12] |
| 228 | 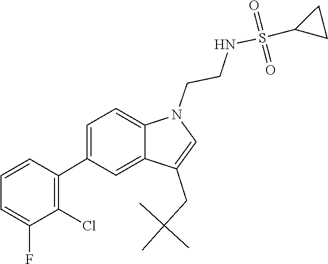 [SMILES: CC(C)(C)Cc1cn(CCNS(=O)(=O)C2CC2)c2ccc(-c3cccc(F)c3Cl)cc12] |
| 229 | 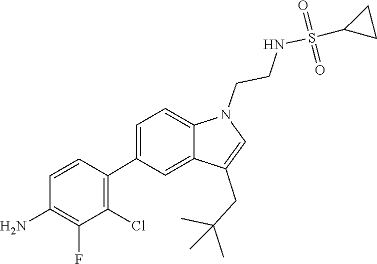 [SMILES: CC(C)(C)Cc1cn(CCNS(=O)(=O)C2CC2)c2ccc(-c3ccc(N)c(F)c3Cl)cc12] |
| 230 | 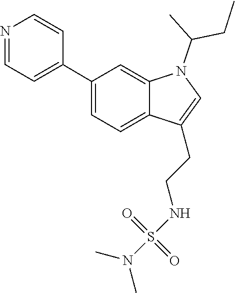 [SMILES: CCC(C)n1cc(CCNS(=O)(=O)N(C)C)c2ccc(-c3ccncc3)cc21] |
| 231 | 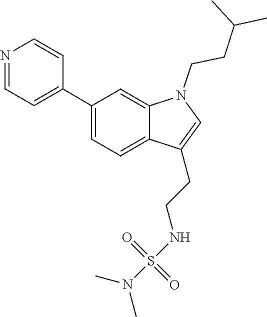 [SMILES: CC(C)CCn1cc(CCNS(=O)(=O)N(C)C)c2ccc(-c3ccncc3)cc21] |
| 232 | 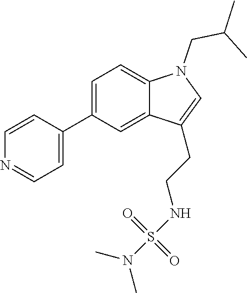 [SMILES: CC(C)Cn1cc(CCNS(=O)(=O)N(C)C)c2cc(-c3ccncc3)ccc21] |
| 233 | 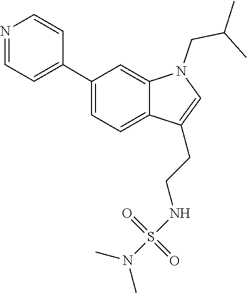 [SMILES: CC(C)Cn1cc(CCNS(=O)(=O)N(C)C)c2ccc(-c3ccncc3)cc21] |
| 234 | 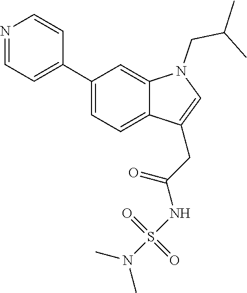 [SMILES: CC(C)Cn1cc(CC(=O)NS(=O)(=O)N(C)C)c2ccc(-c3ccncc3)cc21] |
| 235 | 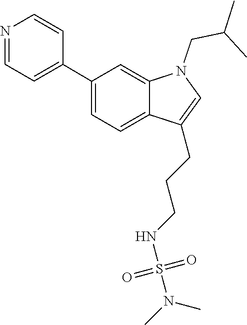 [SMILES: CC(C)Cn1cc(CCCNS(=O)(=O)N(C)C)c2ccc(-c3ccncc3)cc21] |
| 236 | 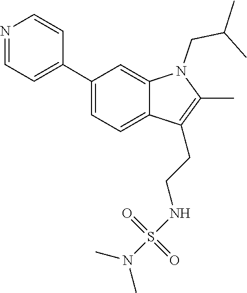 [SMILES: Cc1c(CCNS(=O)(=O)N(C)C)c2ccc(-c3ccncc3)cc2n1CC(C)C] |
| 237 | 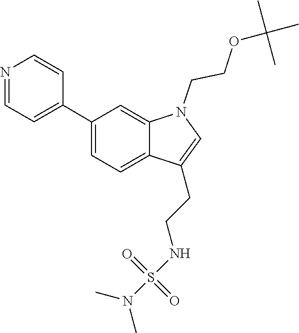 [SMILES: CN(C)S(=O)(=O)NCCc1cn(CCOC(C)(C)C)c2cc(-c3ccncc3)ccc12] |
| 238 | 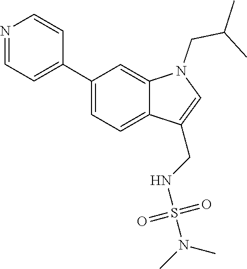 [SMILES: CC(C)Cn1cc(CNS(=O)(=O)N(C)C)c2ccc(-c3ccncc3)cc21] |
| 239 | 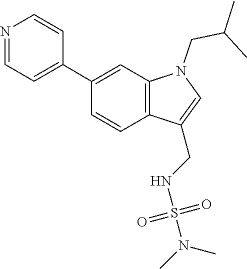 [SMILES: CC(C)Cn1cc(CNS(=O)(=O)N(C)C)c2ccc(-c3ccncc3)cc21] |
| 242 | 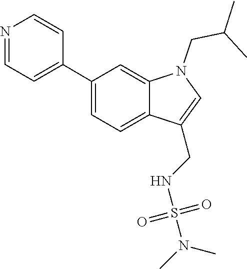 [SMILES: CC(C)Cn1cc(CNS(=O)(=O)N(C)C)c2ccc(-c3ccncc3)cc21] |
| 243 | 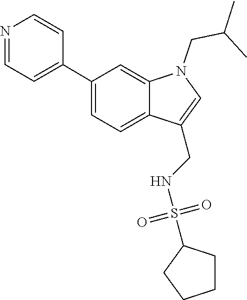 [SMILES: CC(C)Cn1cc(CNS(=O)(=O)C2CCCC2)c2ccc(-c3ccncc3)cc21] |
| 244 | 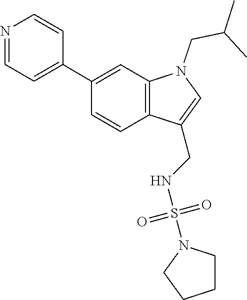 [SMILES: CC(C)Cn1cc(CNS(=O)(=O)N2CCCC2)c2ccc(-c3ccncc3)cc21] |
| 245 | 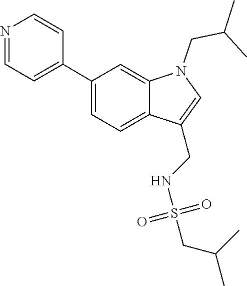 [SMILES: CC(C)Cn1cc(CNS(=O)(=O)CC(C)C)c2ccc(-c3ccncc3)cc21] |
| 246 | 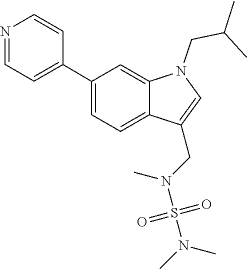 [SMILES: CC(C)Cn1cc(CN(C)S(=O)(=O)N(C)C)c2ccc(-c3ccncc3)cc21] |
| 247 | 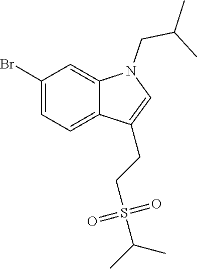 [SMILES: CC(C)Cn1cc(CCS(=O)(=O)C(C)C)c2ccc(Br)cc21] |
| 248 | 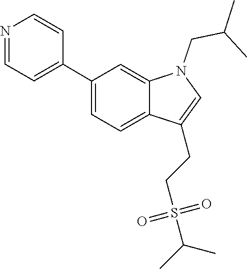 [SMILES: CC(C)Cn1cc(CCS(=O)(=O)C(C)C)c2ccc(-c3ccncc3)cc21] |
| 249 | 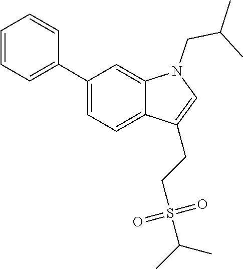 [SMILES: CC(C)Cn1cc(CCS(=O)(=O)C(C)C)c2ccc(-c3ccccc3)cc21] |
| 250 | 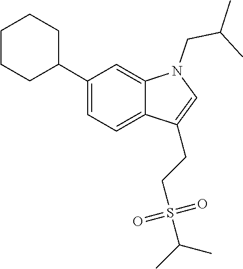 [SMILES: CC(C)Cn1cc(CCS(=O)(=O)C(C)C)c2ccc(C3CCCCC3)cc21] |
| 251 | 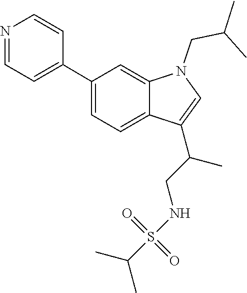 [SMILES: CC(C)Cn1cc(C(C)CNS(=O)(=O)C(C)C)c2ccc(-c3ccncc3)cc21] |
| 252 | 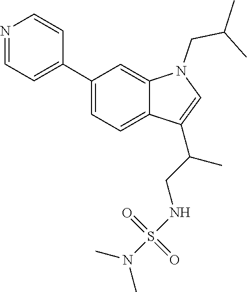 [SMILES: CC(C)Cn1cc(C(C)CNS(=O)(=O)N(C)C)c2ccc(-c3ccncc3)cc21] |
| 253 | 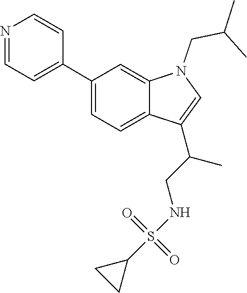 [SMILES: CC(C)Cn1cc(C(C)CNS(=O)(=O)C2CC2)c2ccc(-c3ccncc3)cc21] |
| 254 | 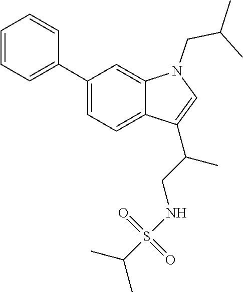 [SMILES: CC(C)Cn1cc(C(C)CNS(=O)(=O)C(C)C)c2ccc(-c3ccccc3)cc21] |
| 255 | 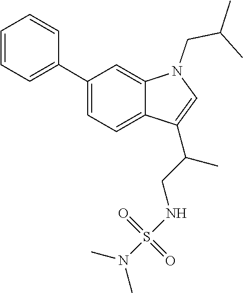 [SMILES: CC(C)Cn1cc(C(C)CNS(=O)(=O)N(C)C)c2ccc(-c3ccccc3)cc21] |
| 256 | 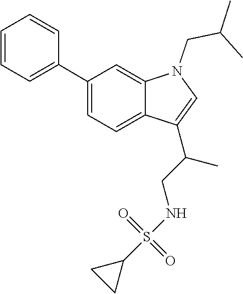 [SMILES: CC(C)Cn1cc(C(C)CNS(=O)(=O)C2CC2)c2ccc(-c3ccccc3)cc21] |
| 257 | 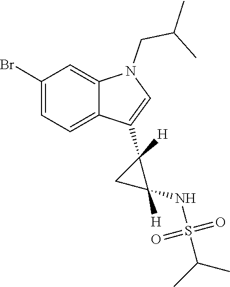 [SMILES: CC(C)Cn1cc([C@H]2C[C@H]2NS(=O)(=O)C(C)C)c2ccc(Br)cc21] |
| 258 | 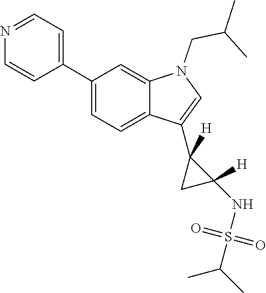 [SMILES: CC(C)Cn1cc([C@H]2C[C@H]2NS(=O)(=O)C(C)C)c2ccc(-c3ccncc3)cc21] |
| 259 | 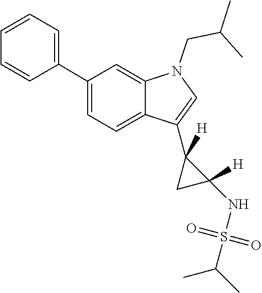 [SMILES: CC(C)Cn1cc([C@H]2C[C@H]2NS(=O)(=O)C(C)C)c2ccc(-c3ccccc3)cc21] |
| 260 | 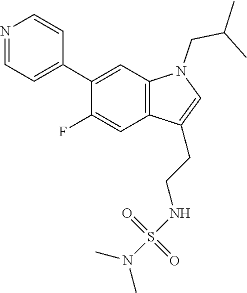 [SMILES: CC(C)Cn1cc(CCNS(=O)(=O)N(C)C)c2cc(F)c(-c3ccncc3)cc21] |
| 261 | 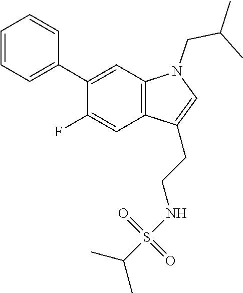 [SMILES: CC(C)Cn1cc(CCNS(=O)(=O)C(C)C)c2cc(F)c(-c3ccccc3)cc21] |
| 262 | 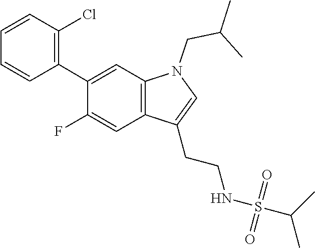 [SMILES: CC(C)Cn1cc(CCNS(=O)(=O)C(C)C)c2cc(F)c(-c3ccccc3Cl)cc21] |
| 263 | 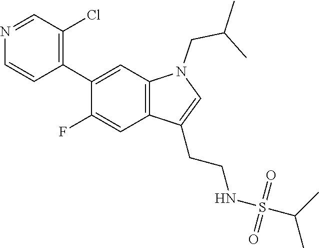 [SMILES: CC(C)Cn1cc(CCNS(=O)(=O)C(C)C)c2cc(F)c(-c3ccncc3Cl)cc21] |
| 264 | 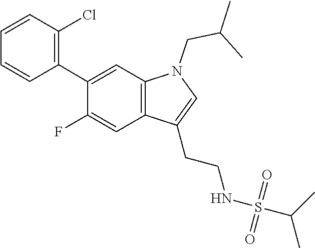 [SMILES: CC(C)Cn1cc(CCNS(=O)(=O)C(C)C)c2cc(F)c(-c3ccccc3Cl)cc21] |
| 265 | 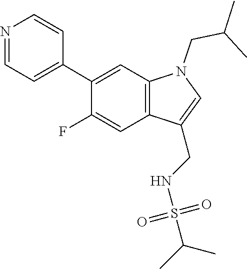 [SMILES: CC(C)Cn1cc(CNS(=O)(=O)C(C)C)c2cc(F)c(-c3ccncc3)cc21] |
| 266 | 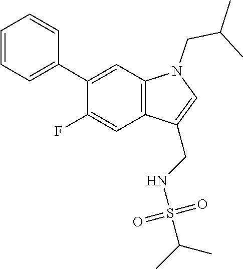 [SMILES: CC(C)Cn1cc(CNS(=O)(=O)C(C)C)c2cc(F)c(-c3ccccc3)cc21] |
| 267 | 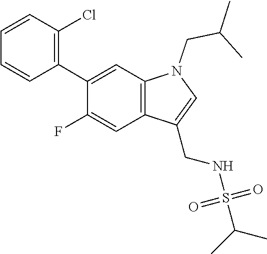 [SMILES: CC(C)Cn1cc(CNS(=O)(=O)C(C)C)c2cc(F)c(-c3ccccc3Cl)cc21] |
| 268 | 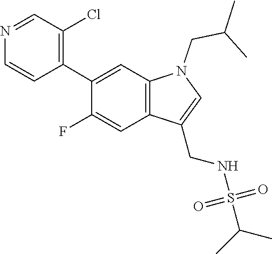 [SMILES: CC(C)Cn1cc(CNS(=O)(=O)C(C)C)c2cc(F)c(-c3ccncc3Cl)cc21] |
| 269 | 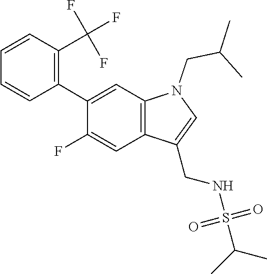 [SMILES: CC(C)Cn1cc(CNS(=O)(=O)C(C)C)c2cc(F)c(-c3ccccc3C(F)(F)F)cc21] |
| 270 | 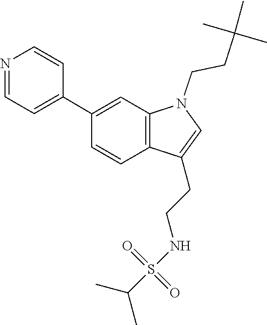 [SMILES: CC(C)S(=O)(=O)NCCc1cn(CCC(C)(C)C)c2cc(-c3ccncc3)ccc12] |
| 271 | 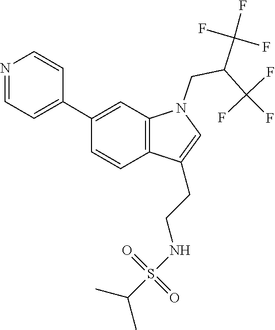 [SMILES: CC(C)S(=O)(=O)NCCc1cn(CC(C(F)(F)F)C(F)(F)F)c2cc(-c3ccncc3)ccc12] |
| 272 | 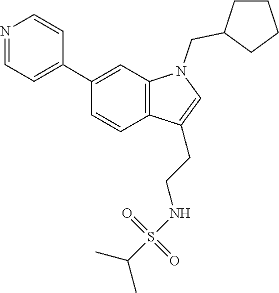 [SMILES: CC(C)S(=O)(=O)NCCc1cn(CC2CCCC2)c2cc(-c3ccncc3)ccc12] |
| 273 | 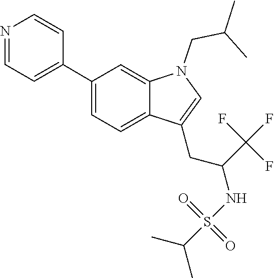 [SMILES: CC(C)Cn1cc(CC(NS(=O)(=O)C(C)C)C(F)(F)F)c2ccc(-c3ccncc3)cc21] |
| 274 | 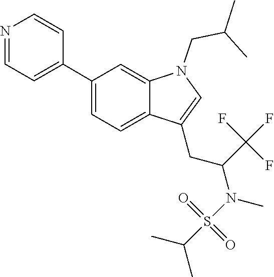 [SMILES: CC(C)Cn1cc(CC(N(C)S(=O)(=O)C(C)C)C(F)(F)F)c2ccc(-c3ccncc3)cc21] |
| 275 | 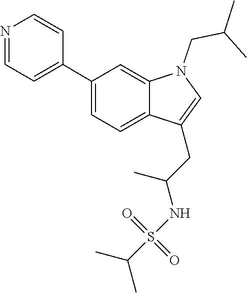 [SMILES: CC(C)Cn1cc(CC(C)NS(=O)(=O)C(C)C)c2ccc(-c3ccncc3)cc21] |
| 276 | 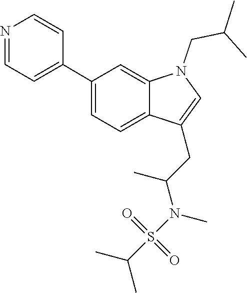 [SMILES: CC(C)Cn1cc(CC(C)N(C)S(=O)(=O)C(C)C)c2ccc(-c3ccncc3)cc21] |
| 277 | 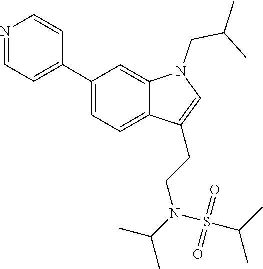 [SMILES: CC(C)Cn1cc(CCN(C(C)C)S(=O)(=O)C(C)C)c2ccc(-c3ccncc3)cc21] |
| 278 | 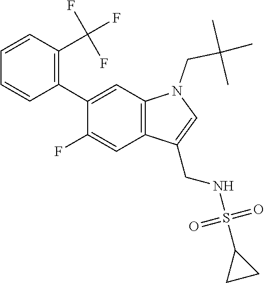 [SMILES: CC(C)(C)Cn1cc(CNS(=O)(=O)C2CC2)c2cc(F)c(-c3ccccc3C(F)(F)F)cc21] |
| 279 | 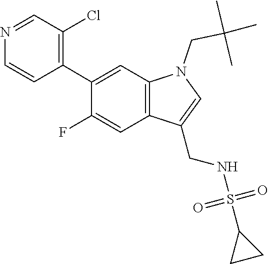 [SMILES: CC(C)(C)Cn1cc(CNS(=O)(=O)C2CC2)c2cc(F)c(-c3ccncc3Cl)cc21] |
| 280 | 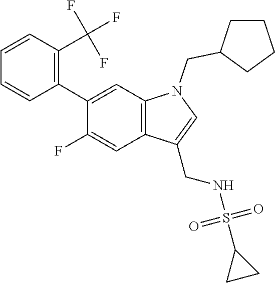 [SMILES: O=S(=O)(NCc1cn(CC2CCCC2)c2cc(-c3ccccc3C(F)(F)F)c(F)cc12)C1CC1] |
| 281 | 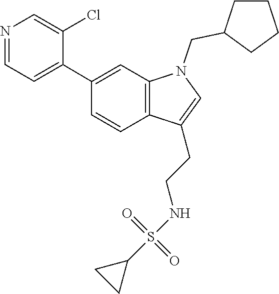 [SMILES: O=S(=O)(NCCc1cn(CC2CCCC2)c2cc(-c3ccncc3Cl)ccc12)C1CC1] |
| 282 | 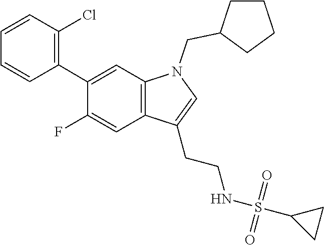 [SMILES: O=S(=O)(NCCc1cn(CC2CCCC2)c2cc(-c3ccccc3Cl)c(F)cc12)C1CC1] |
| 283 | 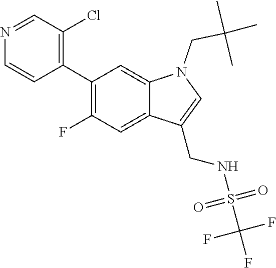 [SMILES: CC(C)(C)Cn1cc(CNS(=O)(=O)C(F)(F)F)c2cc(F)c(-c3ccncc3Cl)cc21] |
| 284 | 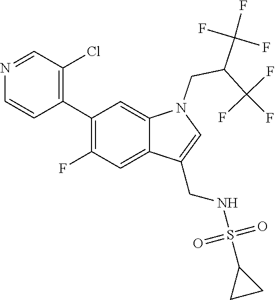 [SMILES: O=S(=O)(NCc1cn(CC(C(F)(F)F)C(F)(F)F)c2cc(-c3ccncc3Cl)c(F)cc12)C1CC1] |
| 285 | 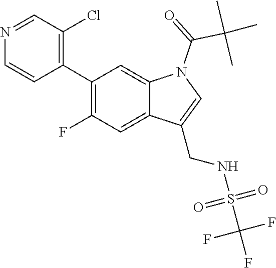 [SMILES: CC(C)(C)C(=O)n1cc(CNS(=O)(=O)C(F)(F)F)c2cc(F)c(-c3ccncc3Cl)cc21] |
| 286 | 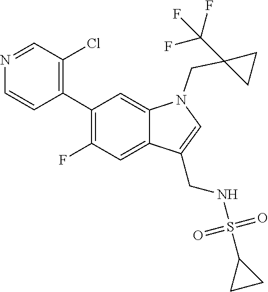 [SMILES: O=S(=O)(NCc1cn(CC2(C(F)(F)F)CC2)c2cc(-c3ccncc3Cl)c(F)cc12)C1CC1] |
| 287 | 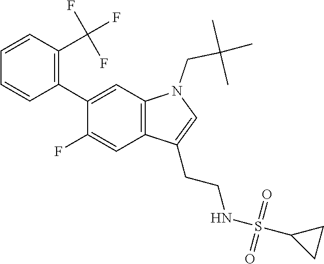 [SMILES: CC(C)(C)Cn1cc(CCNS(=O)(=O)C2CC2)c2cc(F)c(-c3ccccc3C(F)(F)F)cc21] |
| 288 | 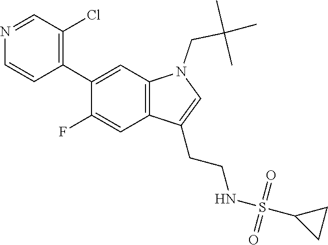 [SMILES: CC(C)(C)Cn1cc(CCNS(=O)(=O)C2CC2)c2cc(F)c(-c3ccncc3Cl)cc21] |
| 289 | 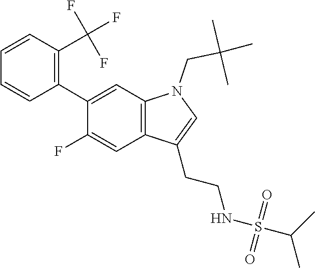 [SMILES: CC(C)S(=O)(=O)NCCc1cn(CC(C)(C)C)c2cc(-c3ccccc3C(F)(F)F)c(F)cc12] |
| 290 | 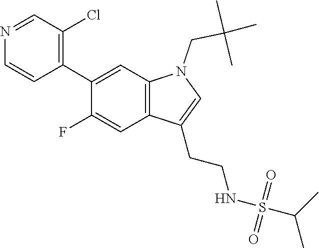 [SMILES: CC(C)S(=O)(=O)NCCc1cn(CC(C)(C)C)c2cc(-c3ccncc3Cl)c(F)cc12] |
| 291 | 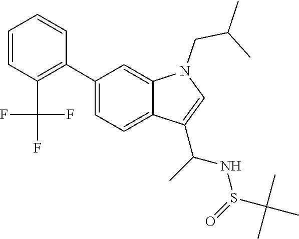 [SMILES: CC(C)Cn1cc(C(C)NS(=O)C(C)(C)C)c2ccc(-c3ccccc3C(F)(F)F)cc21] |
| 292 | 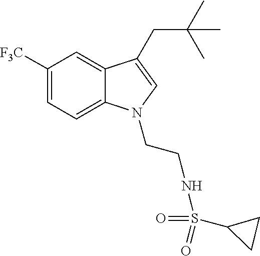 [SMILES: CC(C)(C)Cc1cn(CCNS(=O)(=O)C2CC2)c2ccc(C(F)(F)F)cc12] |
| 293 | 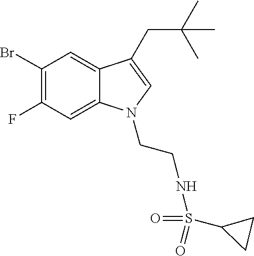 [SMILES: CC(C)(C)Cc1cn(CCNS(=O)(=O)C2CC2)c2cc(F)c(Br)cc12] |
| 294 | 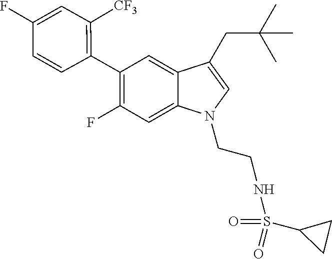 [SMILES: CC(C)(C)Cc1cn(CCNS(=O)(=O)C2CC2)c2cc(F)c(-c3ccc(F)cc3C(F)(F)F)cc12] |
| 295 | 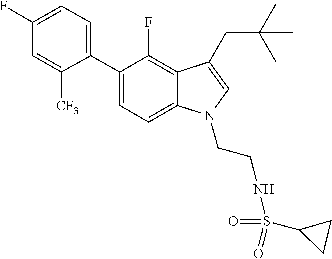 [SMILES: CC(C)(C)Cc1cn(CCNS(=O)(=O)C2CC2)c2ccc(-c3ccc(F)cc3C(F)(F)F)c(F)c12] |
| 296 | 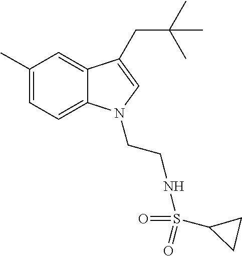 [SMILES: Cc1ccc2c(c1)c(CC(C)(C)C)cn2CCNS(=O)(=O)C1CC1] |
| 297 | 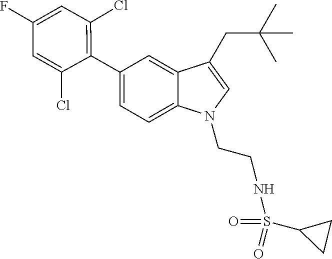 [SMILES: CC(C)(C)Cc1cn(CCNS(=O)(=O)C2CC2)c2ccc(-c3c(Cl)cc(F)cc3Cl)cc12] |
| 298 | 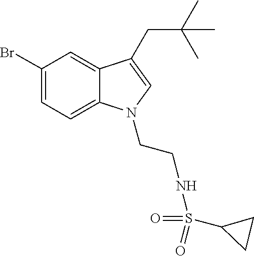 [SMILES: CC(C)(C)Cc1cn(CCNS(=O)(=O)C2CC2)c2ccc(Br)cc12] |
| 299 | 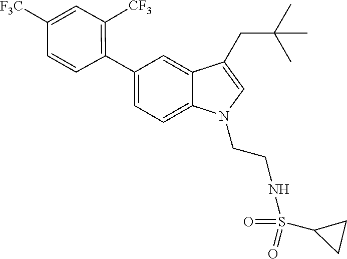 [SMILES: CC(C)(C)Cc1cn(CCNS(=O)(=O)C2CC2)c2ccc(-c3ccc(C(F)(F)F)cc3C(F)(F)F)cc12] |
| 300 | 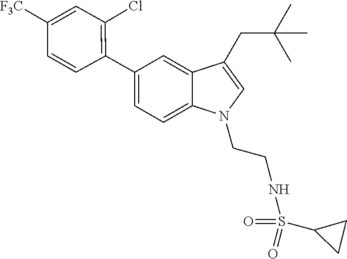 [SMILES: CC(C)(C)Cc1cn(CCNS(=O)(=O)C2CC2)c2ccc(-c3ccc(C(F)(F)F)cc3Cl)cc12] |
| 301 | 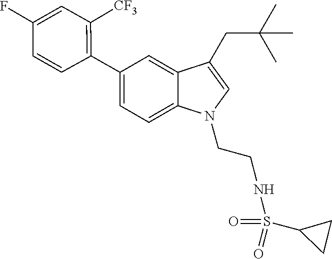 [SMILES: CC(C)(C)Cc1cn(CCNS(=O)(=O)C2CC2)c2ccc(-c3ccc(F)cc3C(F)(F)F)cc12] |
| 302 | 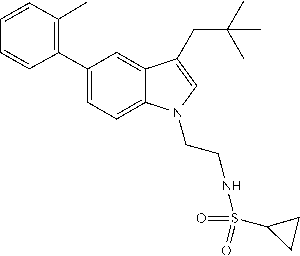 [SMILES: Cc1ccccc1-c1ccc2c(c1)c(CC(C)(C)C)cn2CCNS(=O)(=O)C1CC1] |
| 303 | 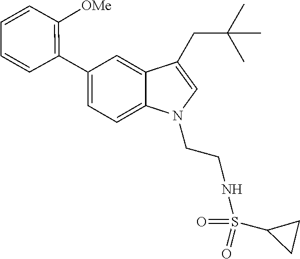 [SMILES: COc1ccccc1-c1ccc2c(c1)c(CC(C)(C)C)cn2CCNS(=O)(=O)C1CC1] |
| 304 | 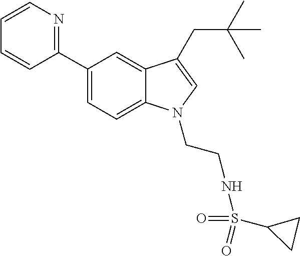 [SMILES: CC(C)(C)Cc1cn(CCNS(=O)(=O)C2CC2)c2ccc(-c3ccccn3)cc12] |
| 305 | 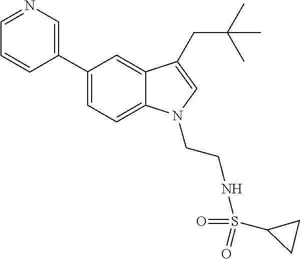 [SMILES: CC(C)(C)Cc1cn(CCNS(=O)(=O)C2CC2)c2ccc(-c3cccnc3)cc12] |
| 306 | 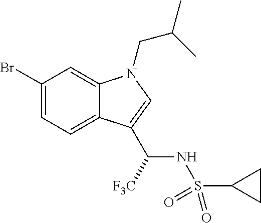 [SMILES: CC(C)Cn1cc([C@H](NS(=O)(=O)C2CC2)C(F)(F)F)c2ccc(Br)cc21] |
| 307 | 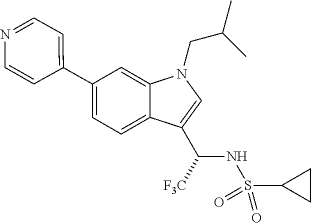 [SMILES: CC(C)Cn1cc([C@H](NS(=O)(=O)C2CC2)C(F)(F)F)c2ccc(-c3ccncc3)cc21] |
| 308 | 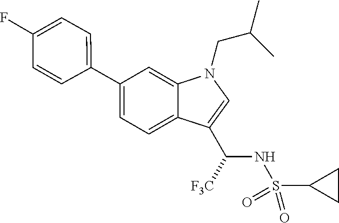 [SMILES: CC(C)Cn1cc([C@H](NS(=O)(=O)C2CC2)C(F)(F)F)c2ccc(-c3ccc(F)cc3)cc21] |
| 309 | 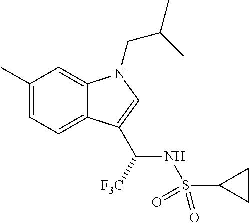 [SMILES: Cc1ccc2c([C@H](NS(=O)(=O)C3CC3)C(F)(F)F)cn(CC(C)C)c2c1] |
| 310 | 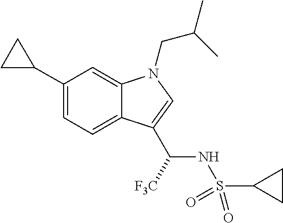 [SMILES: CC(C)Cn1cc([C@H](NS(=O)(=O)C2CC2)C(F)(F)F)c2ccc(C3CC3)cc21] |
| 311 | 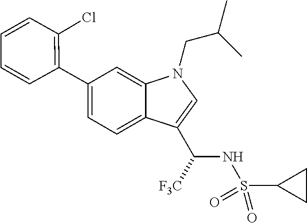 [SMILES: CC(C)Cn1cc([C@H](NS(=O)(=O)C2CC2)C(F)(F)F)c2ccc(-c3ccccc3Cl)cc21] |
| 312 | 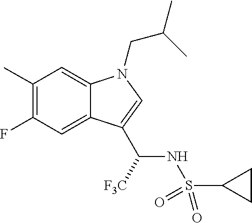 [SMILES: Cc1cc2c(cc1F)c([C@H](NS(=O)(=O)C1CC1)C(F)(F)F)cn2CC(C)C] |
| 313 | 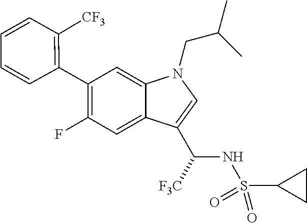 [SMILES: CC(C)Cn1cc([C@H](NS(=O)(=O)C2CC2)C(F)(F)F)c2cc(F)c(-c3ccccc3C(F)(F)F)cc21] |
| 314 | 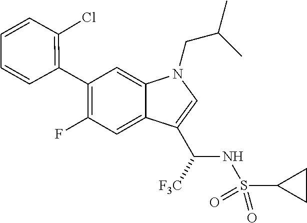 [SMILES: CC(C)Cn1cc([C@H](NS(=O)(=O)C2CC2)C(F)(F)F)c2cc(F)c(-c3ccccc3Cl)cc21] |
| 315 | 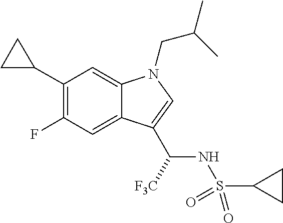 [SMILES: CC(C)Cn1cc([C@H](NS(=O)(=O)C2CC2)C(F)(F)F)c2cc(F)c(C3CC3)cc21] |
| 316 | 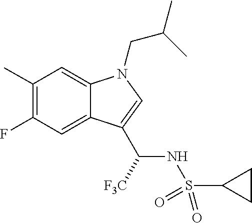 [SMILES: Cc1cc2c(cc1F)c([C@H](NS(=O)(=O)C1CC1)C(F)(F)F)cn2CC(C)C] |
| 317 | 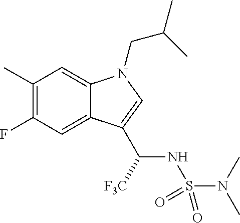 |
| 318 | 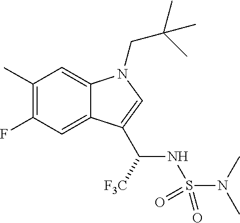 |
| 319 | 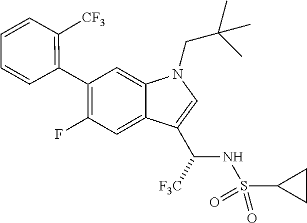 [SMILES: CC(C)(C)Cn1cc([C@H](NS(=O)(=O)C2CC2)C(F)(F)F)c2cc(F)c(-c3ccccc3C(F)(F)F)cc21] |
| 320 | 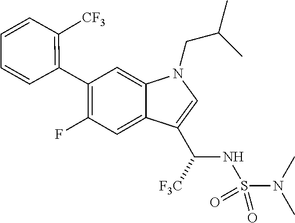 [SMILES: CC(C)Cn1cc([C@H](NS(=O)(=O)N(C)C)C(F)(F)F)c2cc(F)c(-c3ccccc3C(F)(F)F)cc21] |
| 321 | 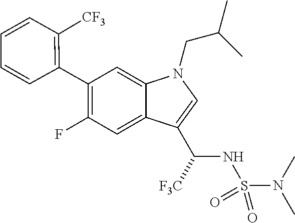 [SMILES: CC(C)Cn1cc([C@H](NS(=O)(=O)N(C)C)C(F)(F)F)c2cc(F)c(-c3ccccc3C(F)(F)F)cc21] |
| 322 | 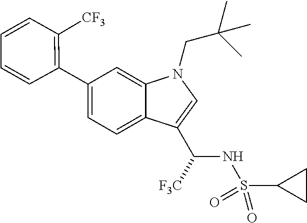 [SMILES: CC(C)(C)Cn1cc([C@H](NS(=O)(=O)C2CC2)C(F)(F)F)c2ccc(-c3ccccc3C(F)(F)F)cc21] |
| 323 | 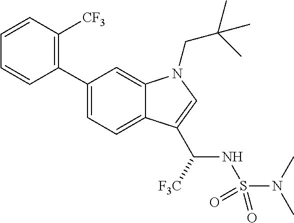 [SMILES: CN(C)S(=O)(=O)N[C@@H](c1cn(CC(C)(C)C)c2cc(-c3ccccc3C(F)(F)F)ccc12)C(F)(F)F] |
| 324 | 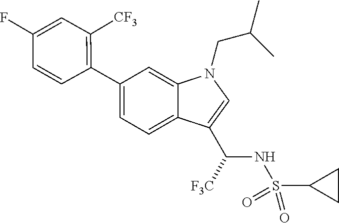 [SMILES: CC(C)Cn1cc([C@H](NS(=O)(=O)C2CC2)C(F)(F)F)c2ccc(-c3ccc(F)cc3C(F)(F)F)cc21] |
| 325 | 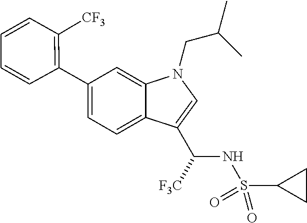 [SMILES: CC(C)Cn1cc([C@H](NS(=O)(=O)C2CC2)C(F)(F)F)c2ccc(-c3ccccc3C(F)(F)F)cc21] |
| 326 | 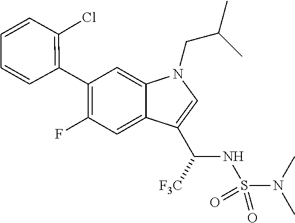 [SMILES: CC(C)Cn1cc([C@H](NS(=O)(=O)N(C)C)C(F)(F)F)c2cc(F)c(-c3ccccc3Cl)cc21] |
| 327 | 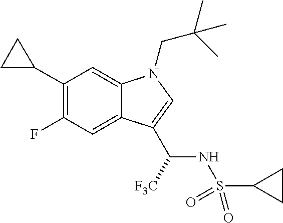 [SMILES: CC(C)(C)Cn1cc([C@H](NS(=O)(=O)C2CC2)C(F)(F)F)c2cc(F)c(C3CC3)cc21] |
| 328 | 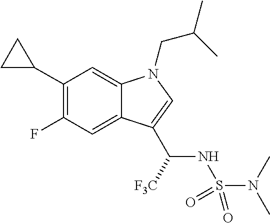 [SMILES: CC(C)Cn1cc([C@H](NS(=O)(=O)N(C)C)C(F)(F)F)c2cc(F)c(C3CC3)cc21] |
| 329 | 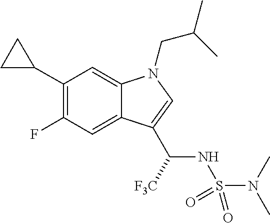 [SMILES: CC(C)Cn1cc([C@H](NS(=O)(=O)N(C)C)C(F)(F)F)c2cc(F)c(C3CC3)cc21] |
| 330 | 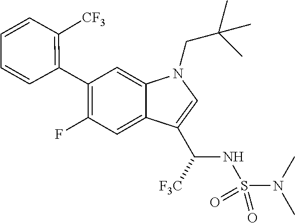 [SMILES: CN(C)S(=O)(=O)N[C@@H](c1cn(CC(C)(C)C)c2cc(-c3ccccc3C(F)(F)F)c(F)cc12)C(F)(F)F] |
| 331 | 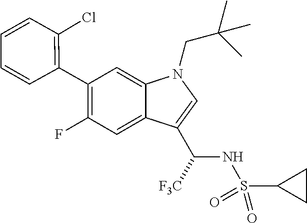 [SMILES: CC(C)(C)Cn1cc([C@H](NS(=O)(=O)C2CC2)C(F)(F)F)c2cc(F)c(-c3ccccc3Cl)cc21] |
| 332 | 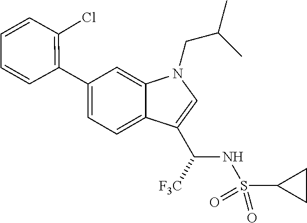 [SMILES: CC(C)Cn1cc([C@H](NS(=O)(=O)C2CC2)C(F)(F)F)c2ccc(-c3ccccc3Cl)cc21] |
| 333 | 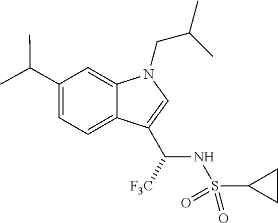 [SMILES: CC(C)Cn1cc([C@H](NS(=O)(=O)C2CC2)C(F)(F)F)c2ccc(C(C)C)cc21] |
| 334 | 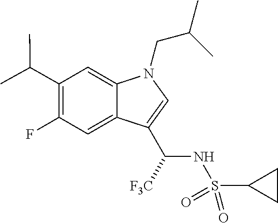 [SMILES: CC(C)Cn1cc([C@H](NS(=O)(=O)C2CC2)C(F)(F)F)c2cc(F)c(C(C)C)cc21] |
| 335 | 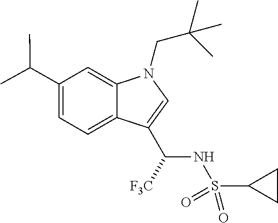 [SMILES: CC(C)c1ccc2c([C@H](NS(=O)(=O)C3CC3)C(F)(F)F)cn(CC(C)(C)C)c2c1] |
| 336 | 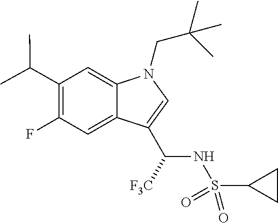 [SMILES: CC(C)c1cc2c(cc1F)c([C@H](NS(=O)(=O)C1CC1)C(F)(F)F)cn2CC(C)(C)C] |
| 337 | 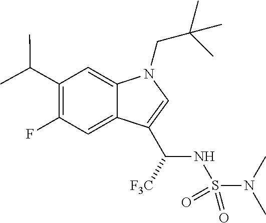 [SMILES: CC(C)c1cc2c(cc1F)c([C@H](NS(=O)(=O)N(C)C)C(F)(F)F)cn2CC(C)(C)C] |
| 338 | 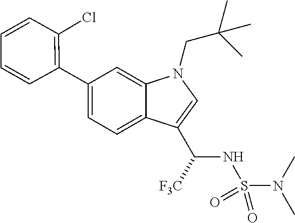 [SMILES: CN(C)S(=O)(=O)N[C@@H](c1cn(CC(C)(C)C)c2cc(-c3ccccc3Cl)ccc12)C(F)(F)F] |
| 339 | 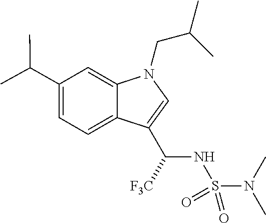 [SMILES: CC(C)Cn1cc([C@H](NS(=O)(=O)N(C)C)C(F)(F)F)c2ccc(C(C)C)cc21] |
| 340 | 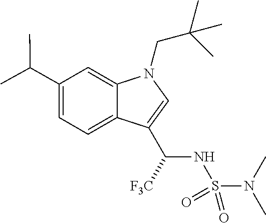 [SMILES: CC(C)c1ccc2c([C@H](NS(=O)(=O)N(C)C)C(F)(F)F)cn(CC(C)(C)C)c2c1] |
| 341 | 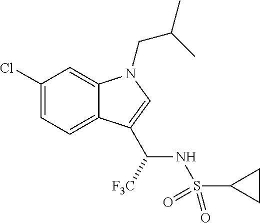 [SMILES: CC(C)Cn1cc([C@H](NS(=O)(=O)C2CC2)C(F)(F)F)c2ccc(Cl)cc21] |
| 342 | 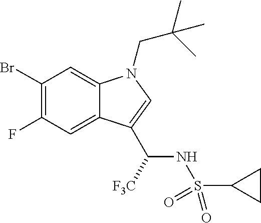 [SMILES: CC(C)(C)Cn1cc([C@H](NS(=O)(=O)C2CC2)C(F)(F)F)c2cc(F)c(Br)cc21] |
| 343 | 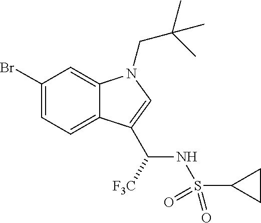 [SMILES: CC(C)(C)Cn1cc([C@H](NS(=O)(=O)C2CC2)C(F)(F)F)c2ccc(Br)cc21] |
| 344 | 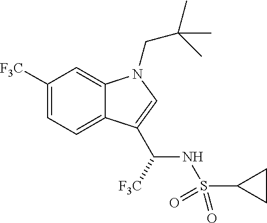 [SMILES: CC(C)(C)Cn1cc([C@H](NS(=O)(=O)C2CC2)C(F)(F)F)c2ccc(C(F)(F)F)cc21] |
| 345 | 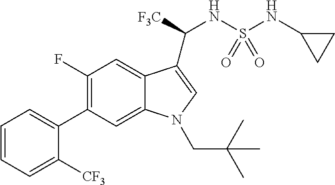 [SMILES: CC(C)(C)Cn1cc([C@H](NS(=O)(=O)NC2CC2)C(F)(F)F)c2cc(F)c(-c3ccccc3C(F)(F)F)cc21] |
| 346 | 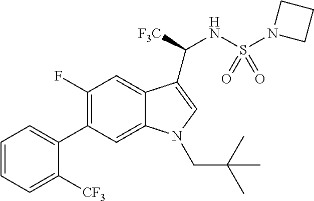 [SMILES: CC(C)(C)Cn1cc([C@H](NS(=O)(=O)N2CCC2)C(F)(F)F)c2cc(F)c(-c3ccccc3C(F)(F)F)cc21] |
| 347 | 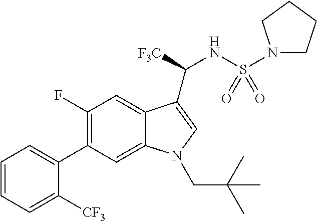 [SMILES: CC(C)(C)Cn1cc([C@H](NS(=O)(=O)N2CCCC2)C(F)(F)F)c2cc(F)c(-c3ccccc3C(F)(F)F)cc21] |
| 348 | 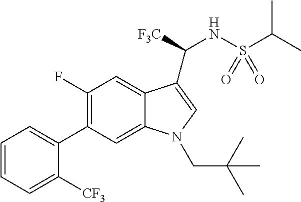 [SMILES: CC(C)S(=O)(=O)N[C@@H](c1cn(CC(C)(C)C)c2cc(-c3ccccc3C(F)(F)F)c(F)cc12)C(F)(F)F] |
| 349 | 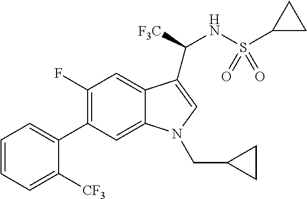 [SMILES: O=S(=O)(N[C@@H](c1cn(CC2CC2)c2cc(-c3ccccc3C(F)(F)F)c(F)cc12)C(F)(F)F)C1CC1] |
| 350 | 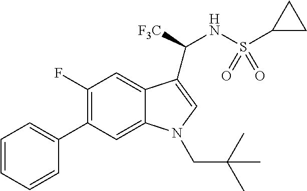 [SMILES: CC(C)(C)Cn1cc([C@H](NS(=O)(=O)C2CC2)C(F)(F)F)c2cc(F)c(-c3ccccc3)cc21] |
| 351 | 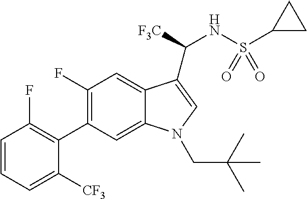 [SMILES: CC(C)(C)Cn1cc([C@H](NS(=O)(=O)C2CC2)C(F)(F)F)c2cc(F)c(-c3c(F)cccc3C(F)(F)F)cc21] |
| 352 | 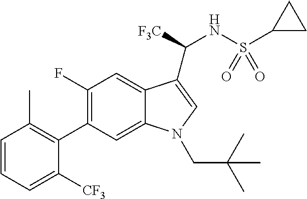 [SMILES: Cc1cccc(C(F)(F)F)c1-c1cc2c(cc1F)c([C@H](NS(=O)(=O)C1CC1)C(F)(F)F)cn2CC(C)(C)C] |
| 353 | 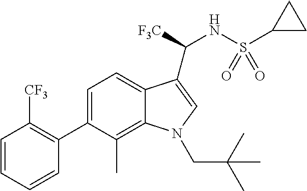 [SMILES: Cc1c(-c2ccccc2C(F)(F)F)ccc2c([C@H](NS(=O)(=O)C3CC3)C(F)(F)F)cn(CC(C)(C)C)c12] |
| 354 | 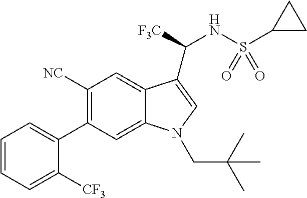 [SMILES: CC(C)(C)Cn1cc([C@H](NS(=O)(=O)C2CC2)C(F)(F)F)c2cc(C#N)c(-c3ccccc3C(F)(F)F)cc21] |
| 355 | 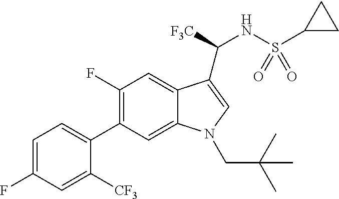 [SMILES: CC(C)(C)Cn1cc([C@H](NS(=O)(=O)C2CC2)C(F)(F)F)c2cc(F)c(-c3ccc(F)cc3C(F)(F)F)cc21] |
| 356 | 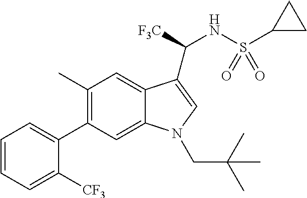 [SMILES: Cc1cc2c([C@H](NS(=O)(=O)C3CC3)C(F)(F)F)cn(CC(C)(C)C)c2cc1-c1ccccc1C(F)(F)F] |
| 357 | 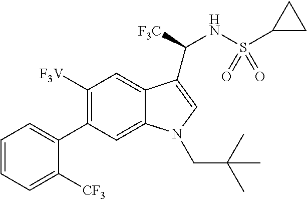 |
| 358 | 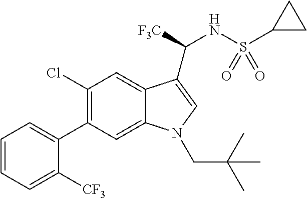 [SMILES: CC(C)(C)Cn1cc([C@H](NS(=O)(=O)C2CC2)C(F)(F)F)c2cc(Cl)c(-c3ccccc3C(F)(F)F)cc21] |
| 359 | 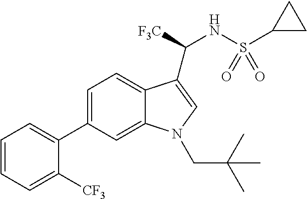 [SMILES: CC(C)(C)Cn1cc([C@H](NS(=O)(=O)C2CC2)C(F)(F)F)c2ccc(-c3ccccc3C(F)(F)F)cc21] |
| 360 | 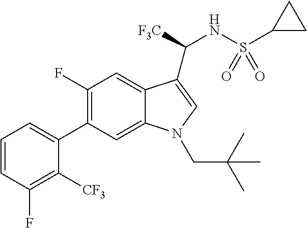 [SMILES: CC(C)(C)Cn1cc([C@H](NS(=O)(=O)C2CC2)C(F)(F)F)c2cc(F)c(-c3cccc(F)c3C(F)(F)F)cc21] |
| 361 | 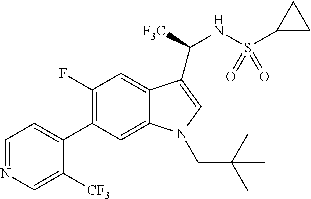 [SMILES: CC(C)(C)Cn1cc([C@H](NS(=O)(=O)C2CC2)C(F)(F)F)c2cc(F)c(-c3ccncc3C(F)(F)F)cc21] |
| 362 | 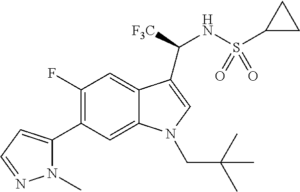 [SMILES: Cn1nccc1-c1cc2c(cc1F)c([C@H](NS(=O)(=O)C1CC1)C(F)(F)F)cn2CC(C)(C)C] |
| 363 | 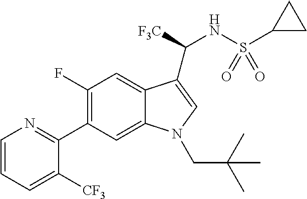 [SMILES: CC(C)(C)Cn1cc([C@H](NS(=O)(=O)C2CC2)C(F)(F)F)c2cc(F)c(-c3ncccc3C(F)(F)F)cc21] |
| 364 | 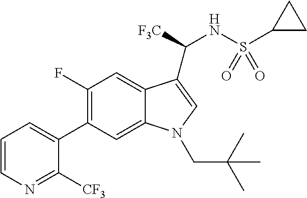 [SMILES: CC(C)(C)Cn1cc([C@H](NS(=O)(=O)C2CC2)C(F)(F)F)c2cc(F)c(-c3cccnc3C(F)(F)F)cc21] |
| 365 | 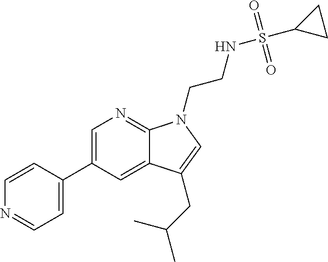 [SMILES: CC(C)Cc1cn(CCNS(=O)(=O)C2CC2)c2ncc(-c3ccncc3)cc12] |

TABLE 1B
Examples of Formula I Compounds

| | |
|---|---|
| 366 | 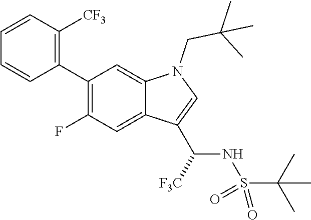 [SMILES: CC(C)(C)Cn1cc([C@H](NS(=O)(=O)C(C)(C)C)C(F)(F)F)c2cc(F)c(-c3ccccc3C(F)(F)F)cc21] |
| 367 | 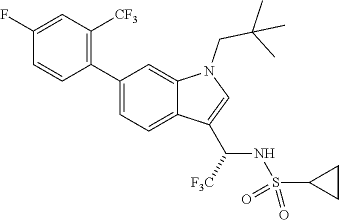 [SMILES: CC(C)(C)Cn1cc([C@H](NS(=O)(=O)C2CC2)C(F)(F)F)c2ccc(-c3ccc(F)cc3C(F)(F)F)cc21] |
| 368 | 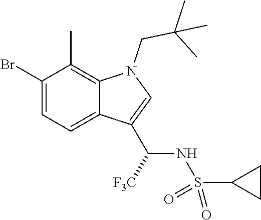 [SMILES: Cc1c(Br)ccc2c([C@H](NS(=O)(=O)C3CC3)C(F)(F)F)cn(CC(C)(C)C)c12] |
| 369 | 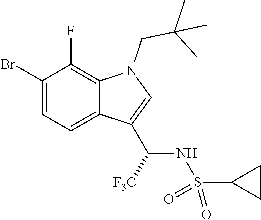 [SMILES: CC(C)(C)Cn1cc([C@H](NS(=O)(=O)C2CC2)C(F)(F)F)c2ccc(Br)c(F)c21] |
| 370 | 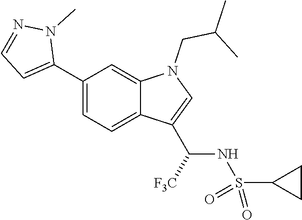 [SMILES: CC(C)Cn1cc([C@H](NS(=O)(=O)C2CC2)C(F)(F)F)c2ccc(-c3ccnn3C)cc21] |
| 371 | 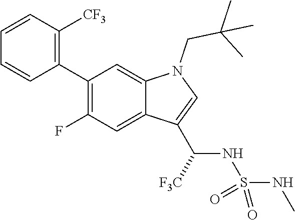 [SMILES: CNS(=O)(=O)N[C@@H](c1cn(CC(C)(C)C)c2cc(-c3ccccc3C(F)(F)F)c(F)cc12)C(F)(F)F] |
| 372 | 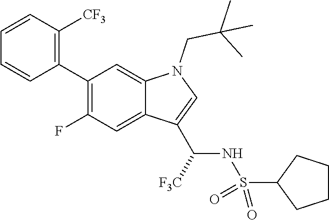 [SMILES: CC(C)(C)Cn1cc([C@H](NS(=O)(=O)C2CCCC2)C(F)(F)F)c2cc(F)c(-c3ccccc3C(F)(F)F)cc21] |
| 373 | 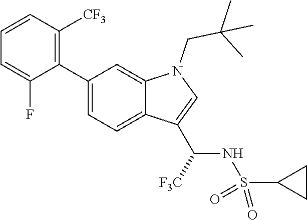 [SMILES: CC(C)(C)Cn1cc([C@H](NS(=O)(=O)C2CC2)C(F)(F)F)c2ccc(-c3c(F)cccc3C(F)(F)F)cc21] |
| 374 | 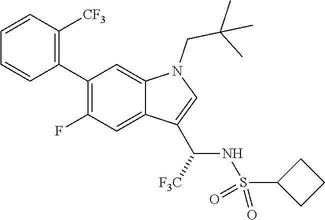 [SMILES: CC(C)(C)Cn1cc([C@H](NS(=O)(=O)C2CCC2)C(F)(F)F)c2cc(F)c(-c3ccccc3C(F)(F)F)cc21] |
| 375 | 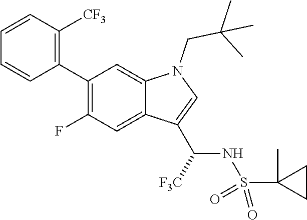 [SMILES: CC(C)(C)Cn1cc([C@H](NS(=O)(=O)C2(C)CC2)C(F)(F)F)c2cc(F)c(-c3ccccc3C(F)(F)F)cc21] |
| 376 | 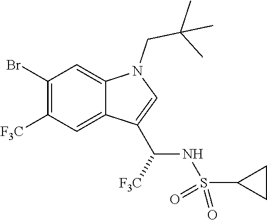 [SMILES: CC(C)(C)Cn1cc([C@H](NS(=O)(=O)C2CC2)C(F)(F)F)c2cc(C(F)(F)F)c(Br)cc21] |
| 377 | 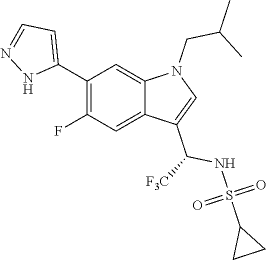 [SMILES: CC(C)Cn1cc([C@H](NS(=O)(=O)C2CC2)C(F)(F)F)c2cc(F)c(-c3ccn[nH]3)cc21] |
| 378 | 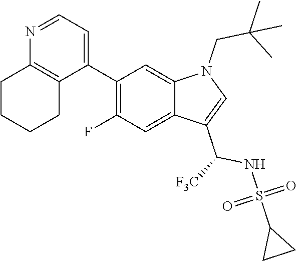 [SMILES: CC(C)(C)Cn1cc([C@H](NS(=O)(=O)C2CC2)C(F)(F)F)c2cc(F)c(-c3ccnc4c3CCCC4)cc21] |
| 379 | 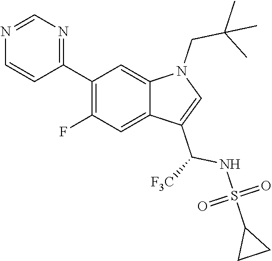 [SMILES: CC(C)(C)Cn1cc([C@H](NS(=O)(=O)C2CC2)C(F)(F)F)c2cc(F)c(-c3ccncn3)cc21] |
| 380 | 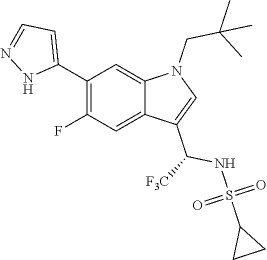 [SMILES: CC(C)(C)Cn1cc([C@H](NS(=O)(=O)C2CC2)C(F)(F)F)c2cc(F)c(-c3ccn[nH]3)cc21] |
| 381 | 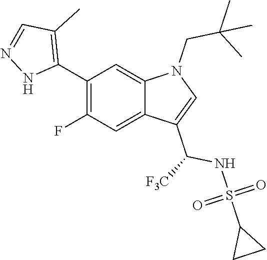 [SMILES: Cc1cn[nH]c1-c1cc2c(cc1F)c([C@H](NS(=O)(=O)C1CC1)C(F)(F)F)cn2CC(C)(C)C] |
| 382 | 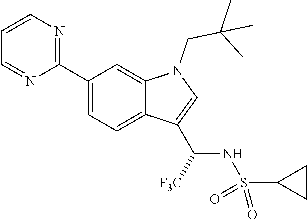 [SMILES: CC(C)(C)Cn1cc([C@H](NS(=O)(=O)C2CC2)C(F)(F)F)c2ccc(-c3ncccn3)cc21] |
| 383 | 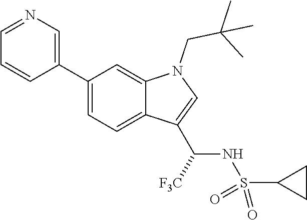 [SMILES: CC(C)(C)Cn1cc([C@H](NS(=O)(=O)C2CC2)C(F)(F)F)c2ccc(-c3cccnc3)cc21] |
| 384 | 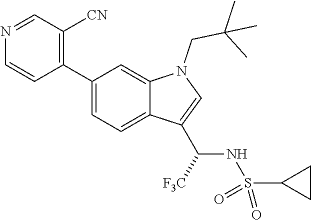 [SMILES: CC(C)(C)Cn1cc([C@H](NS(=O)(=O)C2CC2)C(F)(F)F)c2ccc(-c3ccncc3C#N)cc21] |
| 385 | 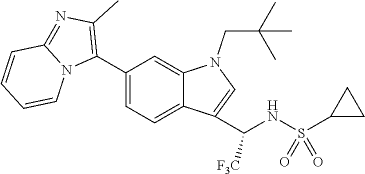 [SMILES: Cc1nc2ccccn2c1-c1ccc2c([C@H](NS(=O)(=O)C3CC3)C(F)(F)F)cn(CC(C)(C)C)c2c1] |
| 386 | 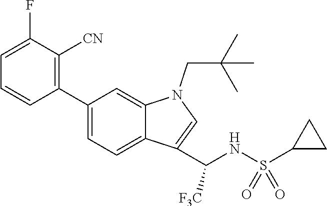 [SMILES: CC(C)(C)Cn1cc([C@H](NS(=O)(=O)C2CC2)C(F)(F)F)c2ccc(-c3cccc(F)c3C#N)cc21] |
| 387 | 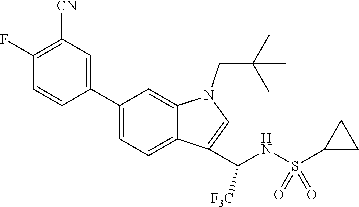 [SMILES: CC(C)(C)Cn1cc([C@H](NS(=O)(=O)C2CC2)C(F)(F)F)c2ccc(-c3ccc(F)c(C#N)c3)cc21] |
| 388 | 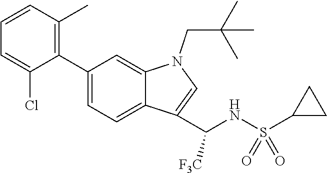 [SMILES: Cc1cccc(Cl)c1-c1ccc2c([C@H](NS(=O)(=O)C3CC3)C(F)(F)F)cn(CC(C)(C)C)c2c1] |
| 389 | 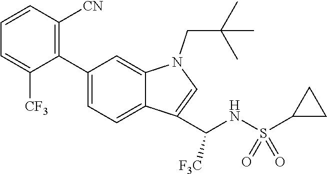 [SMILES: CC(C)(C)Cn1cc([C@H](NS(=O)(=O)C2CC2)C(F)(F)F)c2ccc(-c3c(C#N)cccc3C(F)(F)F)cc21] |
| 390 | 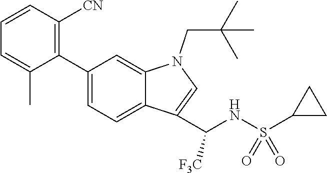 [SMILES: Cc1cccc(C#N)c1-c1ccc2c([C@H](NS(=O)(=O)C3CC3)C(F)(F)F)cn(CC(C)(C)C)c2c1] |
| 391 | 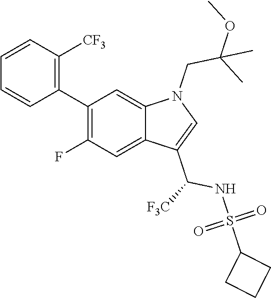 [SMILES: COC(C)(C)Cn1cc([C@H](NS(=O)(=O)C2CCC2)C(F)(F)F)c2cc(F)c(-c3ccccc3C(F)(F)F)cc21] |
| 392 | 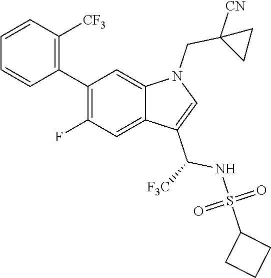 [SMILES: N#CC1(Cn2cc([C@H](NS(=O)(=O)C3CCC3)C(F)(F)F)c3cc(F)c(-c4ccccc4C(F)(F)F)cc32)CC1] |
| 393 | 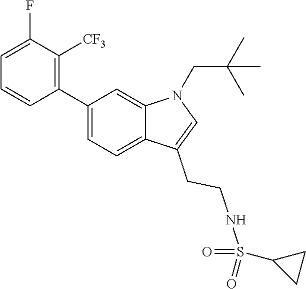 [SMILES: CC(C)(C)Cn1cc(CCNS(=O)(=O)C2CC2)c2ccc(-c3cccc(F)c3C(F)(F)F)cc21] |
| 394 | 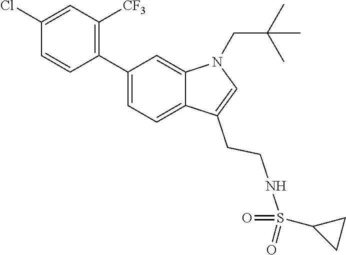 [SMILES: CC(C)(C)Cn1cc(CCNS(=O)(=O)C2CC2)c2ccc(-c3ccc(Cl)cc3C(F)(F)F)cc21] |
| 395 | 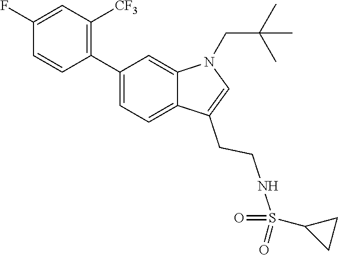 [SMILES: CC(C)(C)Cn1cc(CCNS(=O)(=O)C2CC2)c2ccc(-c3ccc(F)cc3C(F)(F)F)cc21] |
| 396 | 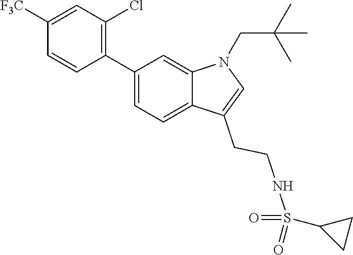 [SMILES: CC(C)(C)Cn1cc(CCNS(=O)(=O)C2CC2)c2ccc(-c3ccc(C(F)(F)F)cc3Cl)cc21] |
| 397 | 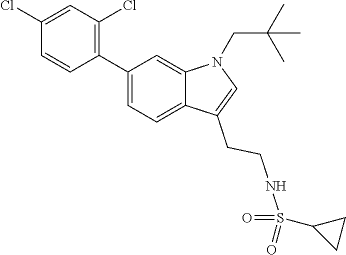 [SMILES: CC(C)(C)Cn1cc(CCNS(=O)(=O)C2CC2)c2ccc(-c3ccc(Cl)cc3Cl)cc21] |
| 398 | 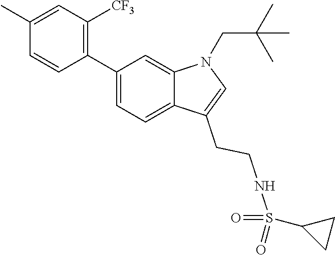 [SMILES: Cc1ccc(-c2ccc3c(CCNS(=O)(=O)C4CC4)cn(CC(C)(C)C)c3c2)c(C(F)(F)F)c1] |
| 399 | 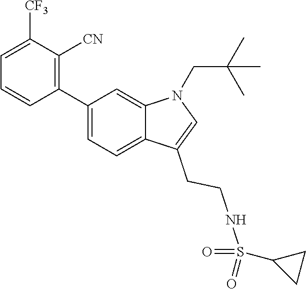 [SMILES: CC(C)(C)Cn1cc(CCNS(=O)(=O)C2CC2)c2ccc(-c3cccc(C(F)(F)F)c3C#N)cc21] |
| 400 | 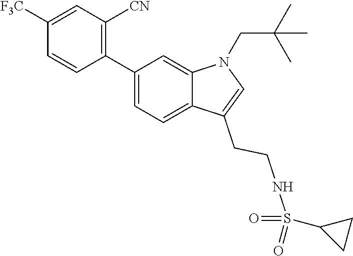 [SMILES: CC(C)(C)Cn1cc(CCNS(=O)(=O)C2CC2)c2ccc(-c3ccc(C(F)(F)F)cc3C#N)cc21] |
| 401 | 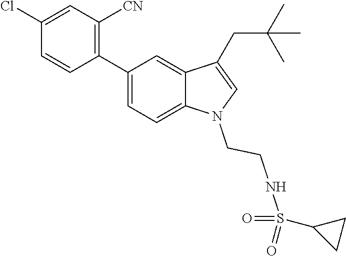 [SMILES: CC(C)(C)Cc1cn(CCNS(=O)(=O)C2CC2)c2ccc(-c3ccc(Cl)cc3C#N)cc12] |
| 402 | 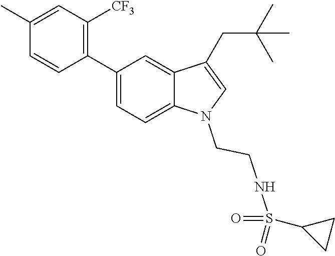 [SMILES: Cc1ccc(-c2ccc3c(c2)c(CC(C)(C)C)cn3CCNS(=O)(=O)C2CC2)c(C(F)(F)F)c1] |
| 403 | 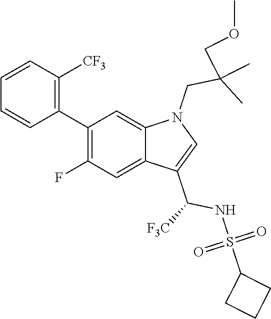 [SMILES: COCC(C)(C)Cn1cc([C@H](NS(=O)(=O)C2CCC2)C(F)(F)F)c2cc(F)c(-c3ccccc3C(F)(F)F)cc21] |
| 404 | 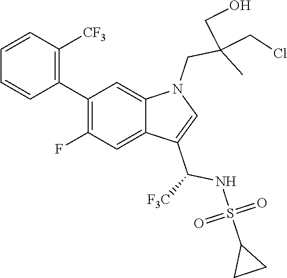 [SMILES: CC(CO)(CCl)Cn1cc([C@H](NS(=O)(=O)C2CC2)C(F)(F)F)c2cc(F)c(-c3ccccc3C(F)(F)F)cc21] |
| 405 | 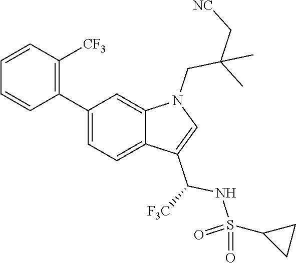 [SMILES: CC(C)(CC#N)Cn1cc([C@H](NS(=O)(=O)C2CC2)C(F)(F)F)c2ccc(-c3ccccc3C(F)(F)F)cc21] |
| 406 | 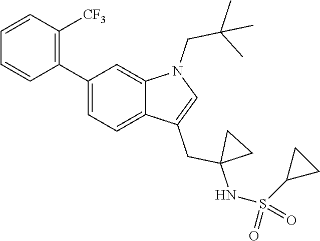 [SMILES: CC(C)(C)Cn1cc(CC2(NS(=O)(=O)C3CC3)CC2)c2ccc(-c3ccccc3C(F)(F)F)cc21] |
| 407 | 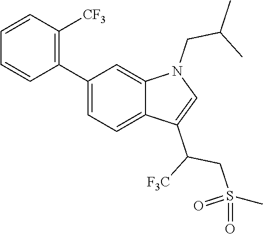 [SMILES: CC(C)Cn1cc(C(CS(C)(=O)=O)C(F)(F)F)c2ccc(-c3ccccc3C(F)(F)F)cc21] |
| 408 | 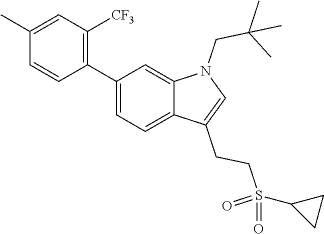 [SMILES: Cc1ccc(-c2ccc3c(CCS(=O)(=O)C4CC4)cn(CC(C)(C)C)c3c2)c(C(F)(F)F)c1] |
| 409 | 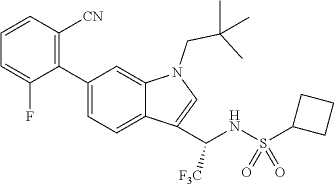 [SMILES: CC(C)(C)Cn1cc([C@H](NS(=O)(=O)C2CCC2)C(F)(F)F)c2ccc(-c3c(F)cccc3C#N)cc21] |
| 410 | 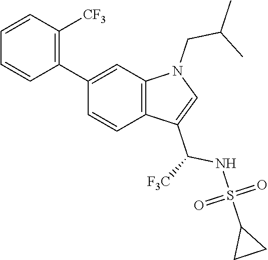 [SMILES: CC(C)Cn1cc([C@H](NS(=O)(=O)C2CC2)C(F)(F)F)c2ccc(-c3ccccc3C(F)(F)F)cc21] |
| 411 | 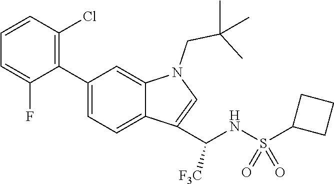 [SMILES: CC(C)(C)Cn1cc([C@H](NS(=O)(=O)C2CCC2)C(F)(F)F)c2ccc(-c3c(F)cccc3Cl)cc21] |
| 412 | 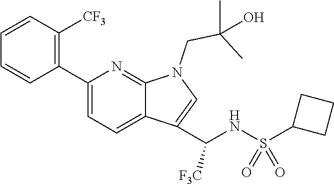 [SMILES: CC(C)(O)Cn1cc([C@H](NS(=O)(=O)C2CCC2)C(F)(F)F)c2ccc(-c3ccccc3C(F)(F)F)nc21] |
| 413 | 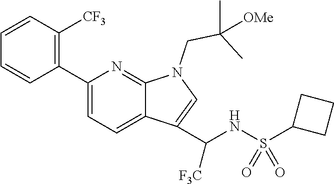 [SMILES: COC(C)(C)Cn1cc(C(NS(=O)(=O)C2CCC2)C(F)(F)F)c2ccc(-c3ccccc3C(F)(F)F)nc21] |
| 414 | 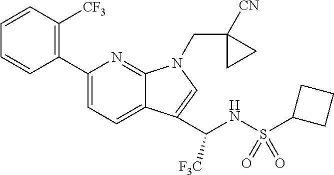 [SMILES: N#CC1(Cn2cc([C@H](NS(=O)(=O)C3CCC3)C(F)(F)F)c3ccc(-c4ccccc4C(F)(F)F)nc32)CC1] |
| 415 | 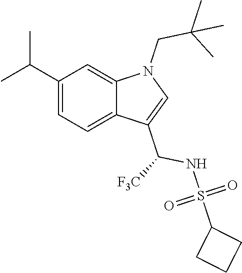 [SMILES: CC(C)c1ccc2c([C@H](NS(=O)(=O)C3CCC3)C(F)(F)F)cn(CC(C)(C)C)c2c1] |
| 416 | 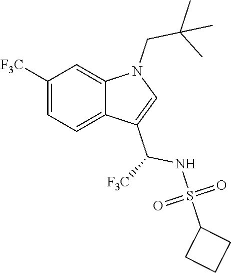 [SMILES: CC(C)(C)Cn1cc([C@H](NS(=O)(=O)C2CCC2)C(F)(F)F)c2ccc(C(F)(F)F)cc21] |
| 417 | 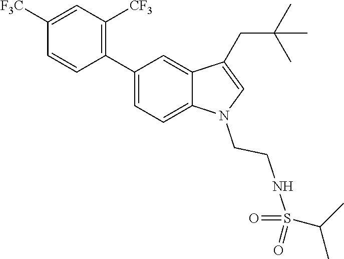 [SMILES: CC(C)S(=O)(=O)NCCn1cc(CC(C)(C)C)c2cc(-c3ccc(C(F)(F)F)cc3C(F)(F)F)ccc21] |
| 418 | 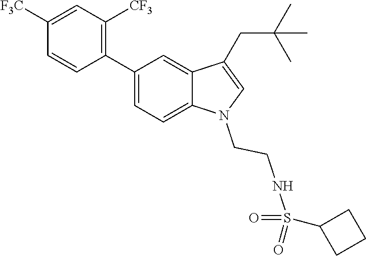 [SMILES: CC(C)(C)Cc1cn(CCNS(=O)(=O)C2CCC2)c2ccc(-c3ccc(C(F)(F)F)cc3C(F)(F)F)cc12] |
| 419 | 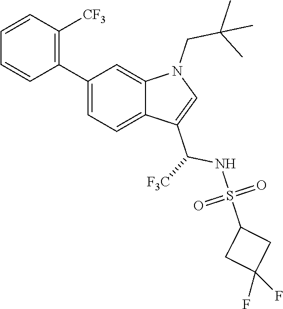 [SMILES: CC(C)(C)Cn1cc([C@H](NS(=O)(=O)C2CC(F)(F)C2)C(F)(F)F)c2ccc(-c3ccccc3C(F)(F)F)cc21] |
| 420 | 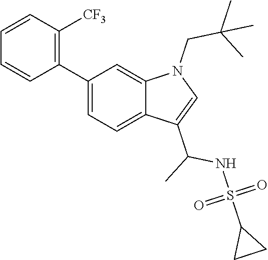 [SMILES: CC(NS(=O)(=O)C1CC1)c1cn(CC(C)(C)C)c2cc(-c3ccccc3C(F)(F)F)ccc12] |
| 421 | 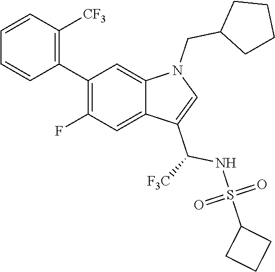 [SMILES: O=S(=O)(N[C@@H](c1cn(CC2CCCC2)c2cc(-c3ccccc3C(F)(F)F)c(F)cc12)C(F)(F)F)C1CCC1] |
| 422 | 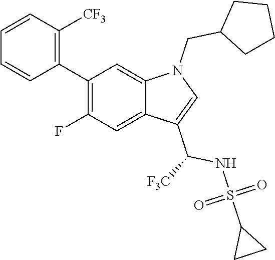 [SMILES: O=S(=O)(N[C@@H](c1cn(CC2CCCC2)c2cc(-c3ccccc3C(F)(F)F)c(F)cc12)C(F)(F)F)C1CC1] |
| 423 | 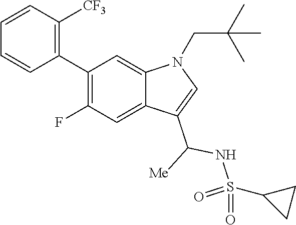 [SMILES: CC(NS(=O)(=O)C1CC1)c1cn(CC(C)(C)C)c2cc(-c3ccccc3C(F)(F)F)c(F)cc12] |
| 424 | 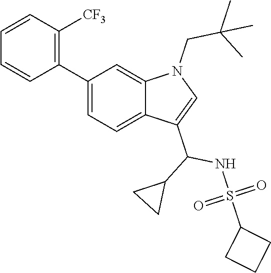 [SMILES: CC(C)(C)Cn1cc(C(NS(=O)(=O)C2CCC2)C2CC2)c2ccc(-c3ccccc3C(F)(F)F)cc21] |
| 425 | 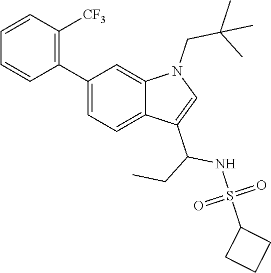 [SMILES: CCC(NS(=O)(=O)C1CCC1)c1cn(CC(C)(C)C)c2cc(-c3ccccc3C(F)(F)F)ccc12] |
| 426 | 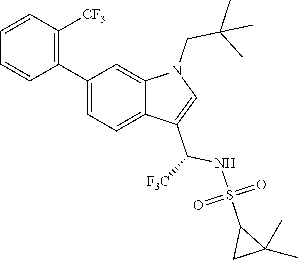 [SMILES: CC(C)(C)Cn1cc([C@H](NS(=O)(=O)C2CC2(C)C)C(F)(F)F)c2ccc(-c3ccccc3C(F)(F)F)cc21] |
| 427 | 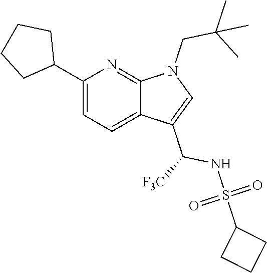 [SMILES: CC(C)(C)Cn1cc([C@H](NS(=O)(=O)C2CCC2)C(F)(F)F)c2ccc(C3CCCC3)nc21] |
| 428 | 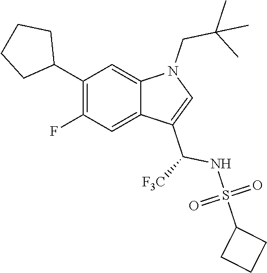 [SMILES: CC(C)(C)Cn1cc([C@H](NS(=O)(=O)C2CCC2)C(F)(F)F)c2cc(F)c(C3CCCC3)cc21] |
| 429 | 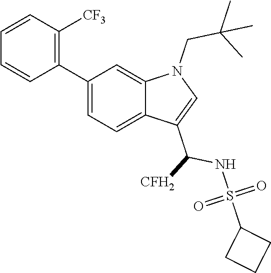 |
| 430 | 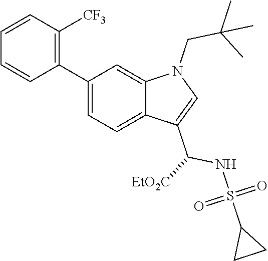 [SMILES: CCOC(=O)[C@@H](NS(=O)(=O)C1CC1)c1cn(CC(C)(C)C)c2cc(-c3ccccc3C(F)(F)F)ccc12] |
| 431 | 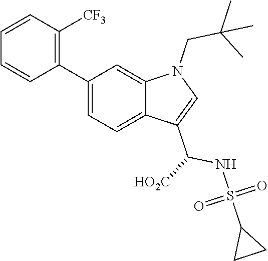 [SMILES: CC(C)(C)Cn1cc([C@H](NS(=O)(=O)C2CC2)C(=O)O)c2ccc(-c3ccccc3C(F)(F)F)cc21] |
| 432 | 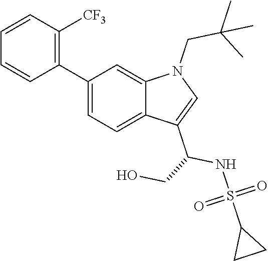 [SMILES: CC(C)(C)Cn1cc([C@@H](CO)NS(=O)(=O)C2CC2)c2ccc(-c3ccccc3C(F)(F)F)cc21] |
| 433 | 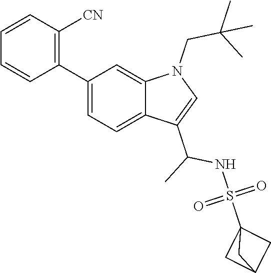 [SMILES: CC(NS(=O)(=O)C12CC(C1)C2)c1cn(CC(C)(C)C)c2cc(-c3ccccc3C#N)ccc12] |
| 434 | 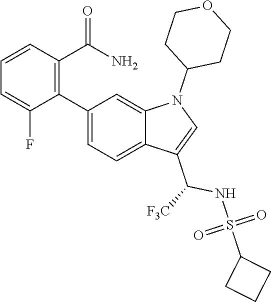 [SMILES: NC(=O)c1cccc(F)c1-c1ccc2c([C@H](NS(=O)(=O)C3CCC3)C(F)(F)F)cn(C3CCOCC3)c2c1] |
| 435 | 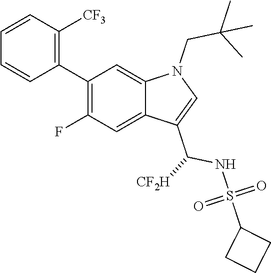 [SMILES: CC(C)(C)Cn1cc([C@H](NS(=O)(=O)C2CCC2)C(F)F)c2cc(F)c(-c3ccccc3C(F)(F)F)cc21] |
| 436 | 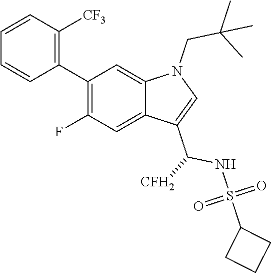 |
| 437 | 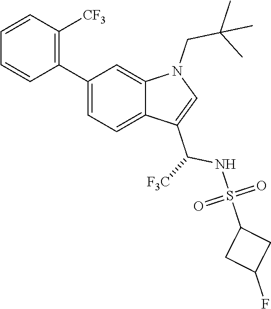 [SMILES: CC(C)(C)Cn1cc([C@H](NS(=O)(=O)C2CC(F)C2)C(F)(F)F)c2ccc(-c3ccccc3C(F)(F)F)cc21] |
| 438 | 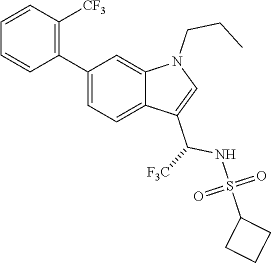 [SMILES: CCCn1cc([C@H](NS(=O)(=O)C2CCC2)C(F)(F)F)c2ccc(-c3ccccc3C(F)(F)F)cc21] |
| 439 | 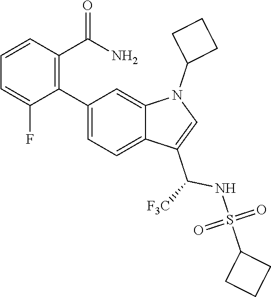 [SMILES: NC(=O)c1cccc(F)c1-c1ccc2c([C@H](NS(=O)(=O)C3CCC3)C(F)(F)F)cn(C3CCC3)c2c1] |
| 440 | 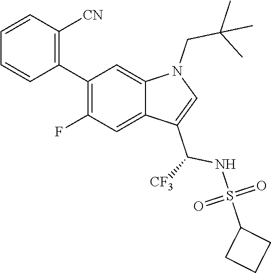 [SMILES: CC(C)(C)Cn1cc([C@H](NS(=O)(=O)C2CCC2)C(F)(F)F)c2cc(F)c(-c3ccccc3C#N)cc21] |
| 441 | 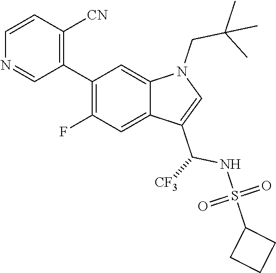 [SMILES: CC(C)(C)Cn1cc([C@H](NS(=O)(=O)C2CCC2)C(F)(F)F)c2cc(F)c(-c3cnccc3C#N)cc21] |
| 442 | 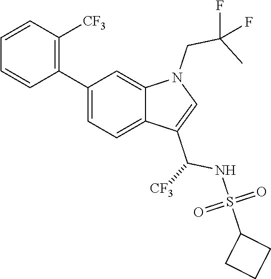 [SMILES: CC(F)(F)Cn1cc([C@H](NS(=O)(=O)C2CCC2)C(F)(F)F)c2ccc(-c3ccccc3C(F)(F)F)cc21] |
| 444 | 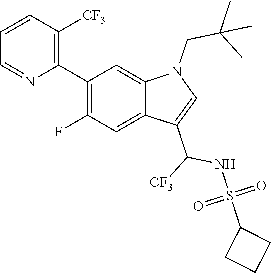 [SMILES: CC(C)(C)Cn1cc(C(NS(=O)(=O)C2CCC2)C(F)(F)F)c2cc(F)c(-c3ncccc3C(F)(F)F)cc21] |
| 445 | 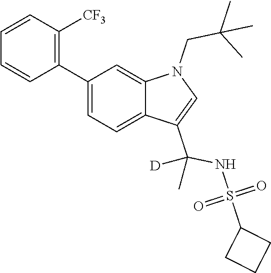 [SMILES: [2H]C(C)(NS(=O)(=O)C1CCC1)c1cn(CC(C)(C)C)c2cc(-c3ccccc3C(F)(F)F)ccc12] |
| 446 | 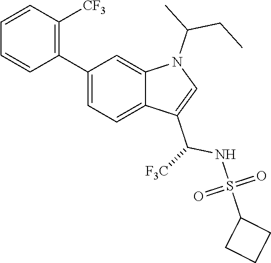 [SMILES: CCC(C)n1cc([C@H](NS(=O)(=O)C2CCC2)C(F)(F)F)c2ccc(-c3ccccc3C(F)(F)F)cc21] |
| 454 | 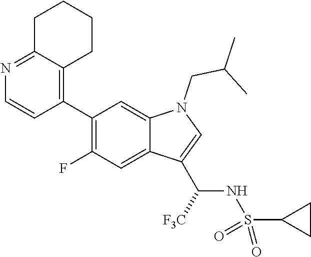 [SMILES: CC(C)Cn1cc([C@H](NS(=O)(=O)C2CC2)C(F)(F)F)c2cc(F)c(-c3ccnc4c3CCCC4)cc21] |
| 455 | 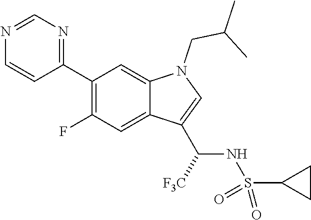 [SMILES: CC(C)Cn1cc([C@H](NS(=O)(=O)C2CC2)C(F)(F)F)c2cc(F)c(-c3ccncn3)cc21] |
| 456 | 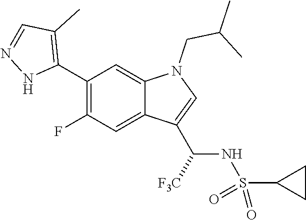 [SMILES: Cc1cn[nH]c1-c1cc2c(cc1F)c([C@H](NS(=O)(=O)C1CC1)C(F)(F)F)cn2CC(C)C] |
| 457 | 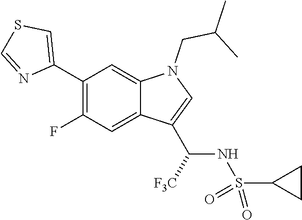 [SMILES: CC(C)Cn1cc([C@H](NS(=O)(=O)C2CC2)C(F)(F)F)c2cc(F)c(-c3cscn3)cc21] |
| 458 | 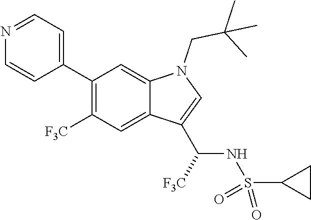 [SMILES: CC(C)(C)Cn1cc([C@H](NS(=O)(=O)C2CC2)C(F)(F)F)c2cc(C(F)(F)F)c(-c3ccncc3)cc21] |
| 459 | 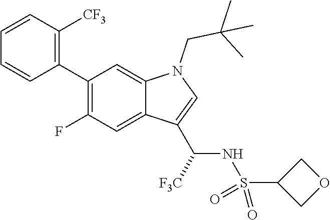 [SMILES: CC(C)(C)Cn1cc([C@H](NS(=O)(=O)C2COC2)C(F)(F)F)c2cc(F)c(-c3ccccc3C(F)(F)F)cc21] |
| 460 | 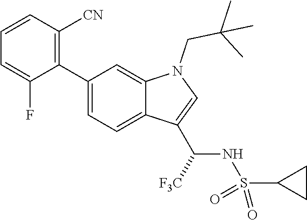 [SMILES: CC(C)(C)Cn1cc([C@H](NS(=O)(=O)C2CC2)C(F)(F)F)c2ccc(-c3c(F)cccc3C#N)cc21] |
| 461 | 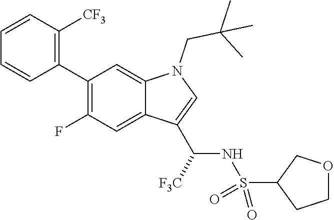 [SMILES: CC(C)(C)Cn1cc([C@H](NS(=O)(=O)C2CCOC2)C(F)(F)F)c2cc(F)c(-c3ccccc3C(F)(F)F)cc21] |
| 462 | 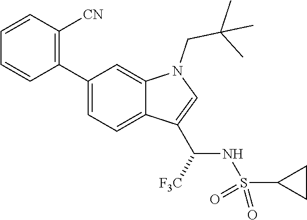 [SMILES: CC(C)(C)Cn1cc([C@H](NS(=O)(=O)C2CC2)C(F)(F)F)c2ccc(-c3ccccc3C#N)cc21] |
| 463 | 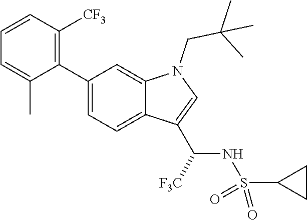 [SMILES: Cc1cccc(C(F)(F)F)c1-c1ccc2c([C@H](NS(=O)(=O)C3CC3)C(F)(F)F)cn(CC(C)(C)C)c2c1] |
| 464 | 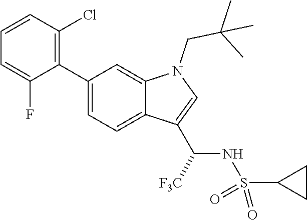 [SMILES: CC(C)(C)Cn1cc([C@H](NS(=O)(=O)C2CC2)C(F)(F)F)c2ccc(-c3c(F)cccc3Cl)cc21] |
| 465 | 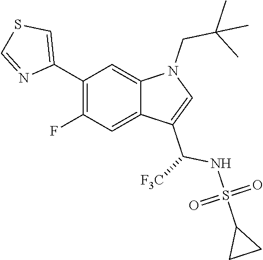 [SMILES: CC(C)(C)Cn1cc([C@H](NS(=O)(=O)C2CC2)C(F)(F)F)c2cc(F)c(-c3cscn3)cc21] |
| 466 | 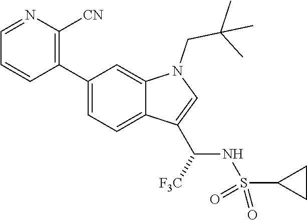 [SMILES: CC(C)(C)Cn1cc([C@H](NS(=O)(=O)C2CC2)C(F)(F)F)c2ccc(-c3cccnc3C#N)cc21] |
| 467 | 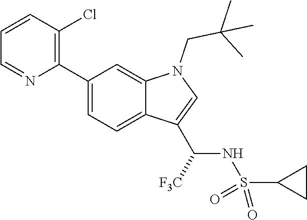 [SMILES: CC(C)(C)Cn1cc([C@H](NS(=O)(=O)C2CC2)C(F)(F)F)c2ccc(-c3ncccc3Cl)cc21] |
| 468 | 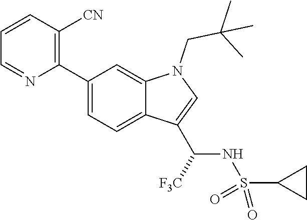 [SMILES: CC(C)(C)Cn1cc([C@H](NS(=O)(=O)C2CC2)C(F)(F)F)c2ccc(-c3ncccc3C#N)cc21] |
| 469 | 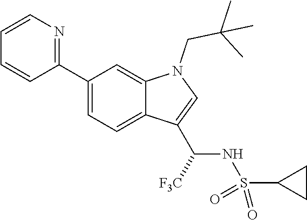 [SMILES: CC(C)(C)Cn1cc([C@H](NS(=O)(=O)C2CC2)C(F)(F)F)c2ccc(-c3ccccn3)cc21] |
| 470 | 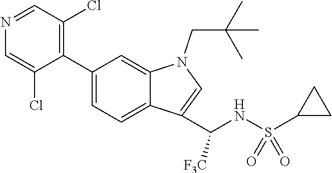 [SMILES: CC(C)(C)Cn1cc([C@H](NS(=O)(=O)C2CC2)C(F)(F)F)c2ccc(-c3c(Cl)cncc3Cl)cc21] |
| 471 | 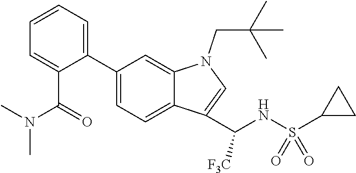 [SMILES: CN(C)C(=O)c1ccccc1-c1ccc2c([C@H](NS(=O)(=O)C3CC3)C(F)(F)F)cn(CC(C)(C)C)c2c1] |
| 472 | 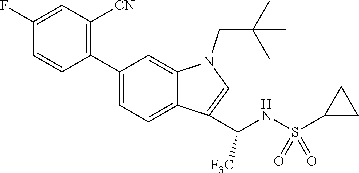 [SMILES: CC(C)(C)Cn1cc([C@H](NS(=O)(=O)C2CC2)C(F)(F)F)c2ccc(-c3ccc(F)cc3C#N)cc21] |
| 473 | 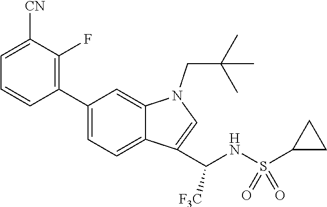 [SMILES: CC(C)(C)Cn1cc([C@H](NS(=O)(=O)C2CC2)C(F)(F)F)c2ccc(-c3cccc(C#N)c3F)cc21] |
| 474 | 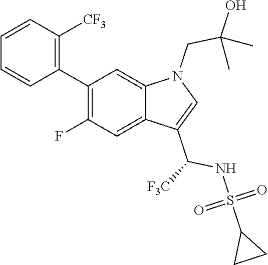 [SMILES: CC(C)(O)Cn1cc([C@H](NS(=O)(=O)C2CC2)C(F)(F)F)c2cc(F)c(-c3ccccc3C(F)(F)F)cc21] |
| 475 | 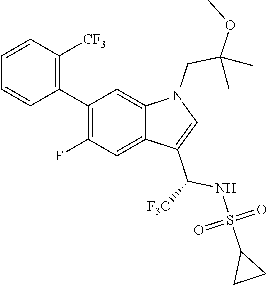 [SMILES: COC(C)(C)Cn1cc([C@H](NS(=O)(=O)C2CC2)C(F)(F)F)c2cc(F)c(-c3ccccc3C(F)(F)F)cc21] |
| 476 | 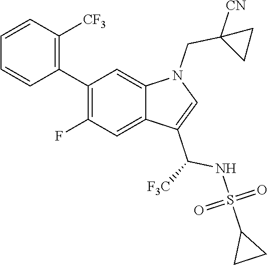 [SMILES: N#CC1(Cn2cc([C@H](NS(=O)(=O)C3CC3)C(F)(F)F)c3cc(F)c(-c4ccccc4C(F)(F)F)cc32)CC1] |
| 477 | 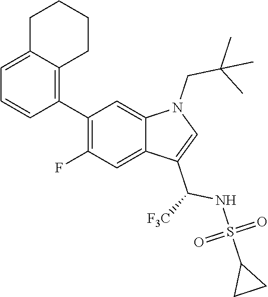 [SMILES: CC(C)(C)Cn1cc([C@H](NS(=O)(=O)C2CC2)C(F)(F)F)c2cc(F)c(-c3cccc4c3CCCC4)cc21] |
| 478 | 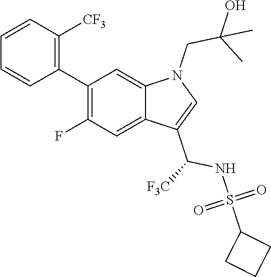 [SMILES: CC(C)(O)Cn1cc([C@H](NS(=O)(=O)C2CCC2)C(F)(F)F)c2cc(F)c(-c3ccccc3C(F)(F)F)cc21] |
| 479 | 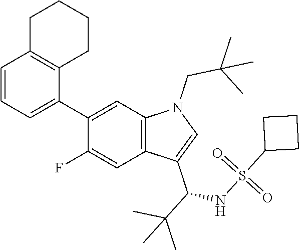 [SMILES: CC(C)(C)Cn1cc([C@H](NS(=O)(=O)C2CCC2)C(C)(C)C)c2cc(F)c(-c3cccc4c3CCCC4)cc21] |
| 480 | 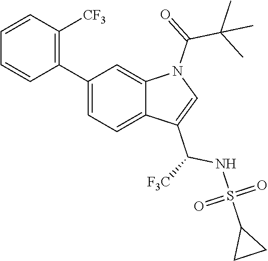 [SMILES: CC(C)(C)C(=O)n1cc([C@H](NS(=O)(=O)C2CC2)C(F)(F)F)c2ccc(-c3ccccc3C(F)(F)F)cc21] |
| 481 | 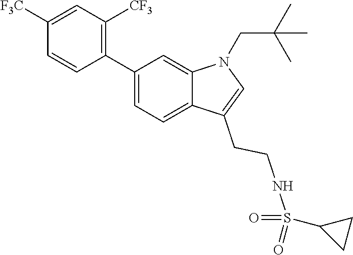 [SMILES: CC(C)(C)Cn1cc(CCNS(=O)(=O)C2CC2)c2ccc(-c3ccc(C(F)(F)F)cc3C(F)(F)F)cc21] |
| 482 | 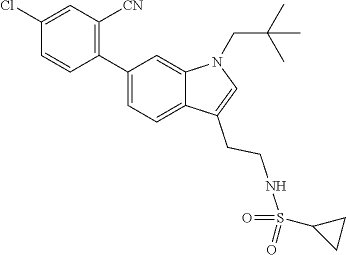 [SMILES: CC(C)(C)Cn1cc(CCNS(=O)(=O)C2CC2)c2ccc(-c3ccc(Cl)cc3C#N)cc21] |
| 483 | 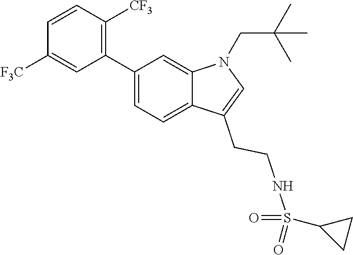 [SMILES: CC(C)(C)Cn1cc(CCNS(=O)(=O)C2CC2)c2ccc(-c3cc(C(F)(F)F)ccc3C(F)(F)F)cc21] |
| 484 | 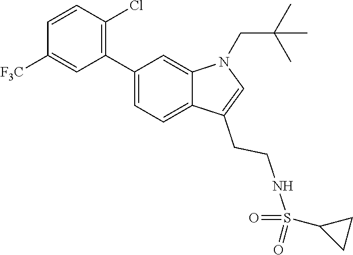 [SMILES: CC(C)(C)Cn1cc(CCNS(=O)(=O)C2CC2)c2ccc(-c3cc(C(F)(F)F)ccc3Cl)cc21] |
| 485 | 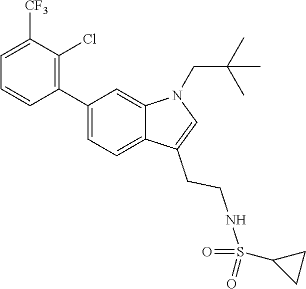 [SMILES: CC(C)(C)Cn1cc(CCNS(=O)(=O)C2CC2)c2ccc(-c3cccc(C(F)(F)F)c3Cl)cc21] |
| 486 | 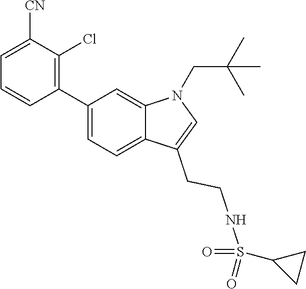 [SMILES: CC(C)(C)Cn1cc(CCNS(=O)(=O)C2CC2)c2ccc(-c3cccc(C#N)c3Cl)cc21] |
| 487 | 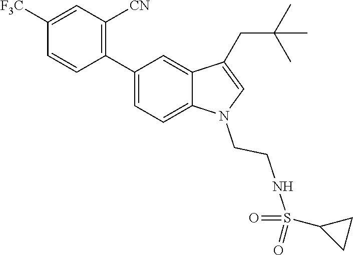 [SMILES: CC(C)(C)Cc1cn(CCNS(=O)(=O)C2CC2)c2ccc(-c3ccc(C(F)(F)F)cc3C#N)cc12] |
| 488 | 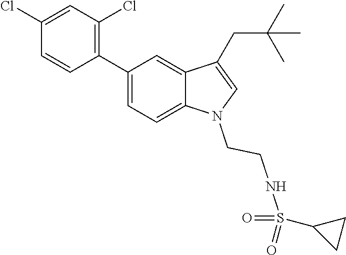 [SMILES: CC(C)(C)Cc1cn(CCNS(=O)(=O)C2CC2)c2ccc(-c3ccc(Cl)cc3Cl)cc12] |
| 489 | 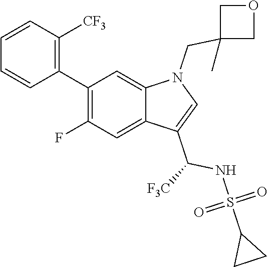 [SMILES: CC1(Cn2cc([C@H](NS(=O)(=O)C3CC3)C(F)(F)F)c3cc(F)c(-c4ccccc4C(F)(F)F)cc32)COC1] |
| 490 | 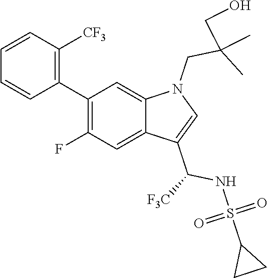 [SMILES: CC(C)(CO)Cn1cc([C@H](NS(=O)(=O)C2CC2)C(F)(F)F)c2cc(F)c(-c3ccccc3C(F)(F)F)cc21] |
| 491 | 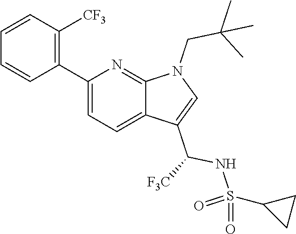 [SMILES: CC(C)(C)Cn1cc([C@H](NS(=O)(=O)C2CC2)C(F)(F)F)c2ccc(-c3ccccc3C(F)(F)F)nc21] |
| 492 | 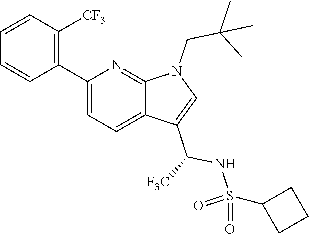 [SMILES: CC(C)(C)Cn1cc([C@H](NS(=O)(=O)C2CCC2)C(F)(F)F)c2ccc(-c3ccccc3C(F)(F)F)nc21] |
| 493 | 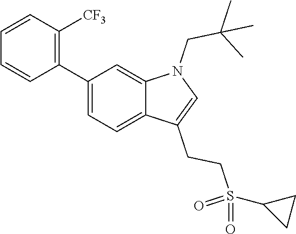 [SMILES: CC(C)(C)Cn1cc(CCS(=O)(=O)C2CC2)c2ccc(-c3ccccc3C(F)(F)F)cc21] |
| 494 | 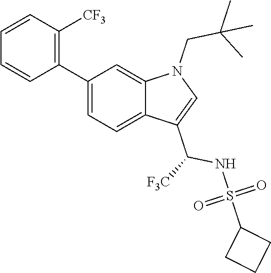 [SMILES: CC(C)(C)Cn1cc([C@H](NS(=O)(=O)C2CCC2)C(F)(F)F)c2ccc(-c3ccccc3C(F)(F)F)cc21] |
| 495 | 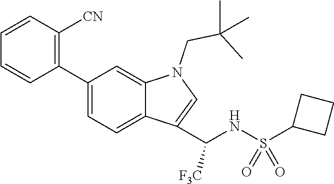 [SMILES: CC(C)(C)Cn1cc([C@H](NS(=O)(=O)C2CCC2)C(F)(F)F)c2ccc(-c3ccccc3C#N)cc21] |
| 496 | 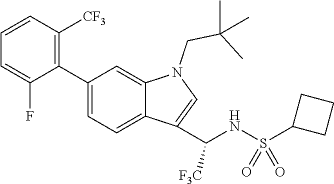 [SMILES: CC(C)(C)Cn1cc([C@H](NS(=O)(=O)C2CCC2)C(F)(F)F)c2ccc(-c3c(F)cccc3C(F)(F)F)cc21] |
| 497 | 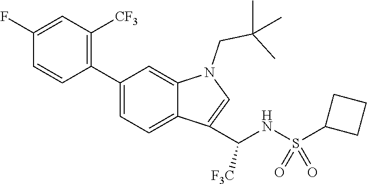 [SMILES: CC(C)(C)Cn1cc([C@H](NS(=O)(=O)C2CCC2)C(F)(F)F)c2ccc(-c3ccc(F)cc3C(F)(F)F)cc21] |
| 498 | 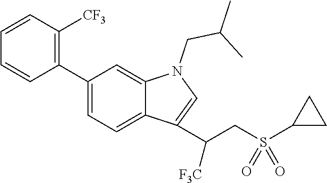 [SMILES: CC(C)Cn1cc(C(CS(=O)(=O)C2CC2)C(F)(F)F)c2ccc(-c3ccccc3C(F)(F)F)cc21] |
| 499 | 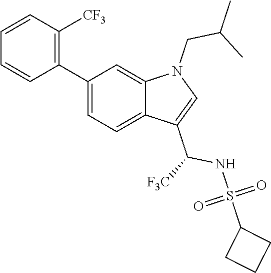 [SMILES: CC(C)Cn1cc([C@H](NS(=O)(=O)C2CCC2)C(F)(F)F)c2ccc(-c3ccccc3C(F)(F)F)cc21] |
| 500 | 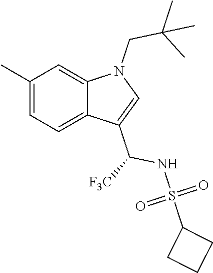 [SMILES: Cc1ccc2c([C@H](NS(=O)(=O)C3CCC3)C(F)(F)F)cn(CC(C)(C)C)c2c1] |
| 501 | 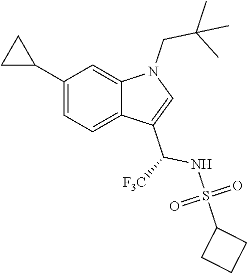 [SMILES: CC(C)(C)Cn1cc([C@H](NS(=O)(=O)C2CCC2)C(F)(F)F)c2ccc(C3CC3)cc21] |
| 502 | 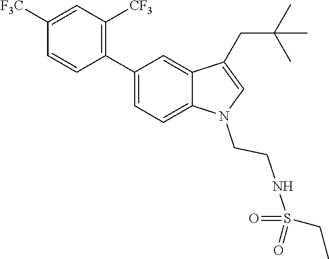 [SMILES: CCS(=O)(=O)NCCn1cc(CC(C)(C)C)c2cc(-c3ccc(C(F)(F)F)cc3C(F)(F)F)ccc21] |
| 503 | 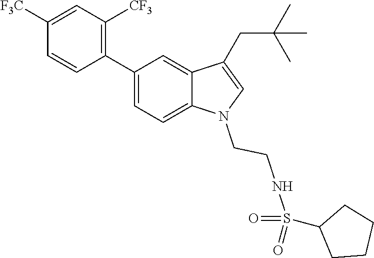 [SMILES: CC(C)(C)Cc1cn(CCNS(=O)(=O)C2CCCC2)c2ccc(-c3ccc(C(F)(F)F)cc3C(F)(F)F)cc12] |
| 504 | 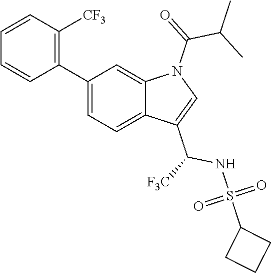 [SMILES: CC(C)C(=O)n1cc([C@H](NS(=O)(=O)C2CCC2)C(F)(F)F)c2ccc(-c3ccccc3C(F)(F)F)cc21] |
| 505 | 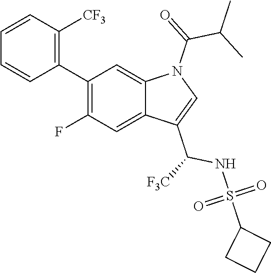 [SMILES: CC(C)C(=O)n1cc([C@H](NS(=O)(=O)C2CCC2)C(F)(F)F)c2cc(F)c(-c3ccccc3C(F)(F)F)cc21] |
| 506 | 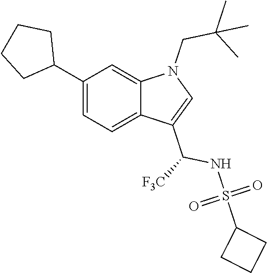 [SMILES: CC(C)(C)Cn1cc([C@H](NS(=O)(=O)C2CCC2)C(F)(F)F)c2ccc(C3CCCC3)cc21] |
| 507 | 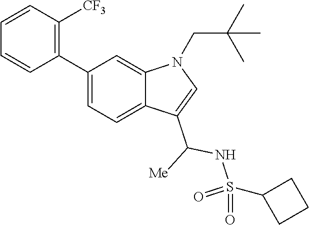 [SMILES: CC(NS(=O)(=O)C1CCC1)c1cn(CC(C)(C)C)c2cc(-c3ccccc3C(F)(F)F)ccc12] |
| 508 | 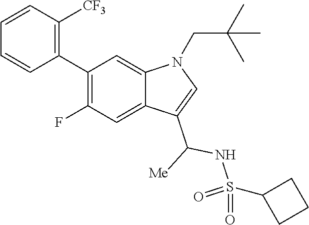 [SMILES: CC(NS(=O)(=O)C1CCC1)c1cn(CC(C)(C)C)c2cc(-c3ccccc3C(F)(F)F)c(F)cc12] |
| 509 | 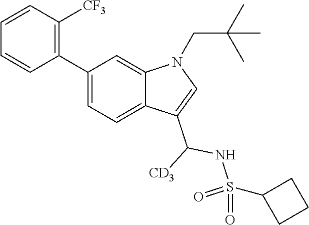 [SMILES: [2H]C([2H])([2H])C(NS(=O)(=O)C1CCC1)c1cn(CC(C)(C)C)c2cc(-c3ccccc3C(F)(F)F)ccc12] |
| 510 | 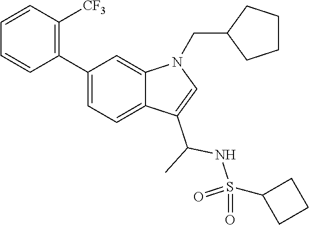 [SMILES: CC(NS(=O)(=O)C1CCC1)c1cn(CC2CCCC2)c2cc(-c3ccccc3C(F)(F)F)ccc12] |
| 511 | 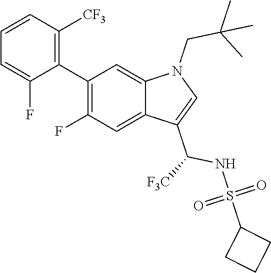 [SMILES: CC(C)(C)Cn1cc([C@H](NS(=O)(=O)C2CCC2)C(F)(F)F)c2cc(F)c(-c3c(F)cccc3C(F)(F)F)cc21] |
| 512 | 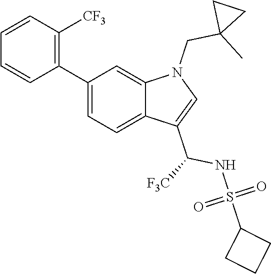 [SMILES: CC1(Cn2cc([C@H](NS(=O)(=O)C3CCC3)C(F)(F)F)c3ccc(-c4ccccc4C(F)(F)F)cc32)CC1] |
| 513 | 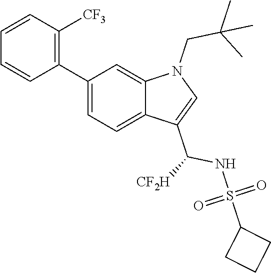 [SMILES: CC(C)(C)Cn1cc([C@H](NS(=O)(=O)C2CCC2)C(F)F)c2ccc(-c3ccccc3C(F)(F)F)cc21] |
| 514 | 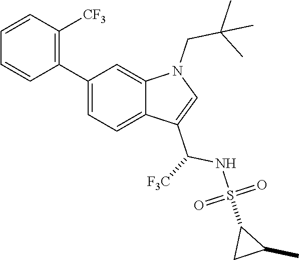 |
| 515 | 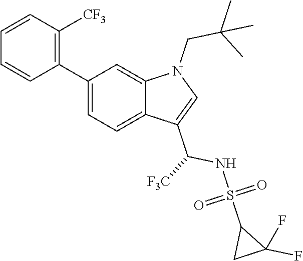 [SMILES: CC(C)(C)Cn1cc([C@H](NS(=O)(=O)C2CC2(F)F)C(F)(F)F)c2ccc(-c3ccccc3C(F)(F)F)cc21] |
| 516 | 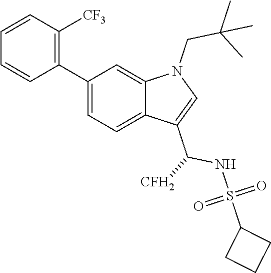 |
| 517 | 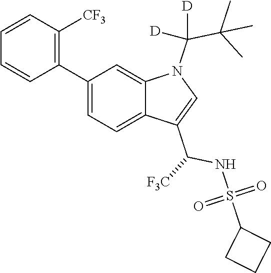 [SMILES: [2H]C([2H])(n1cc([C@H](NS(=O)(=O)C2CCC2)C(F)(F)F)c2ccc(-c3ccccc3C(F)(F)F)cc21)C(C)(C)C] |
| 518 | 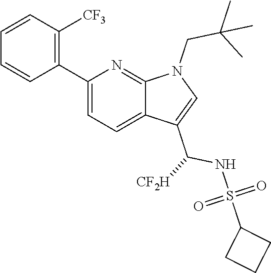 [SMILES: CC(C)(C)Cn1cc([C@H](NS(=O)(=O)C2CCC2)C(F)F)c2ccc(-c3ccccc3C(F)(F)F)nc21] |
| 519 | 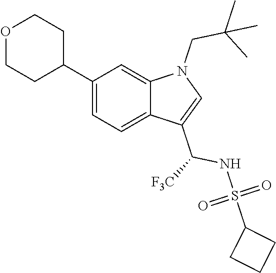 [SMILES: CC(C)(C)Cn1cc([C@H](NS(=O)(=O)C2CCC2)C(F)(F)F)c2ccc(C3CCOCC3)cc21] |
| 520 | 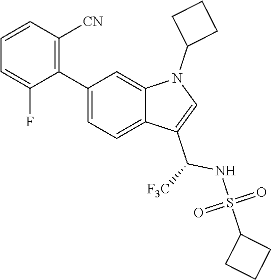 [SMILES: N#Cc1cccc(F)c1-c1ccc2c([C@H](NS(=O)(=O)C3CCC3)C(F)(F)F)cn(C3CCC3)c2c1] |
| 521 | 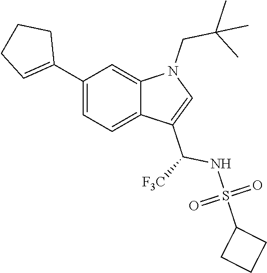 [SMILES: CC(C)(C)Cn1cc([C@H](NS(=O)(=O)C2CCC2)C(F)(F)F)c2ccc(C3=CCCC3)cc21] |
| 522 | 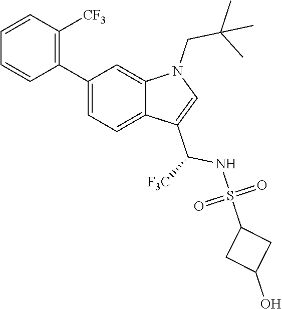 [SMILES: CC(C)(C)Cn1cc([C@H](NS(=O)(=O)C2CC(O)C2)C(F)(F)F)c2ccc(-c3ccccc3C(F)(F)F)cc21] |
| 523 | 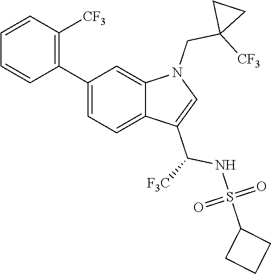 [SMILES: O=S(=O)(N[C@@H](c1cn(CC2(C(F)(F)F)CC2)c2cc(-c3ccccc3C(F)(F)F)ccc12)C(F)(F)F)C1CCC1] |
| 524 | 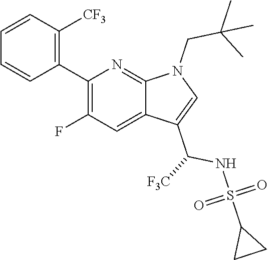 [SMILES: CC(C)(C)Cn1cc([C@H](NS(=O)(=O)C2CC2)C(F)(F)F)c2cc(F)c(-c3ccccc3C(F)(F)F)nc21] |
| 525 | 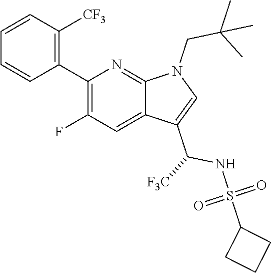 [SMILES: CC(C)(C)Cn1cc([C@H](NS(=O)(=O)C2CCC2)C(F)(F)F)c2cc(F)c(-c3ccccc3C(F)(F)F)nc21] |
| 526 | 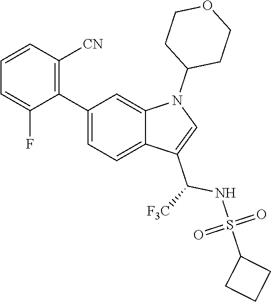 [SMILES: N#Cc1cccc(F)c1-c1ccc2c([C@H](NS(=O)(=O)C3CCC3)C(F)(F)F)cn(C3CCOCC3)c2c1] |
| 527 | 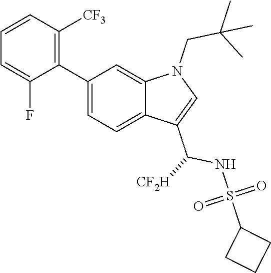 [SMILES: CC(C)(C)Cn1cc([C@H](NS(=O)(=O)C2CCC2)C(F)F)c2ccc(-c3c(F)cccc3C(F)(F)F)cc21] |
| 528 | 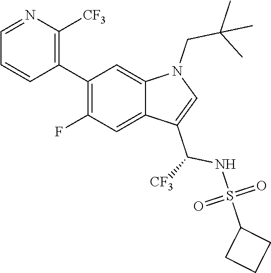 [SMILES: CC(C)(C)Cn1cc([C@H](NS(=O)(=O)C2CCC2)C(F)(F)F)c2cc(F)c(-c3cccnc3C(F)(F)F)cc21] |
| 529 | 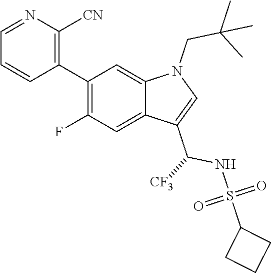 [SMILES: CC(C)(C)Cn1cc([C@H](NS(=O)(=O)C2CCC2)C(F)(F)F)c2cc(F)c(-c3cccnc3C#N)cc21] |
| 530 | 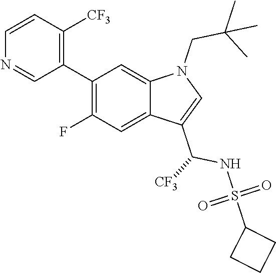 [SMILES: CC(C)(C)Cn1cc([C@H](NS(=O)(=O)C2CCC2)C(F)(F)F)c2cc(F)c(-c3cnccc3C(F)(F)F)cc21] |
| 533 | 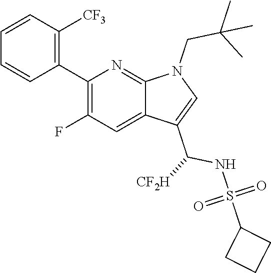 [SMILES: CC(C)(C)Cn1cc([C@H](NS(=O)(=O)C2CCC2)C(F)F)c2cc(F)c(-c3ccccc3C(F)(F)F)nc21] |
| 534 | 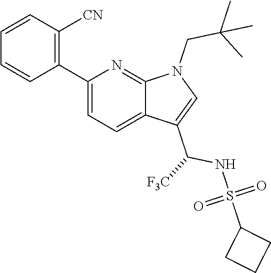 [SMILES: CC(C)(C)Cn1cc([C@H](NS(=O)(=O)C2CCC2)C(F)(F)F)c2ccc(-c3ccccc3C#N)nc21] |
| 535 | 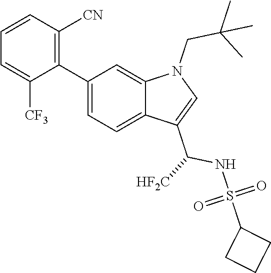 |
| 536 | 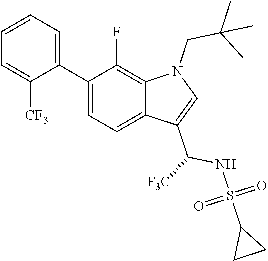 [SMILES: CC(C)(C)Cn1cc([C@H](NS(=O)(=O)C2CC2)C(F)(F)F)c2ccc(-c3ccccc3C(F)(F)F)c(F)c21] |
| 537 | 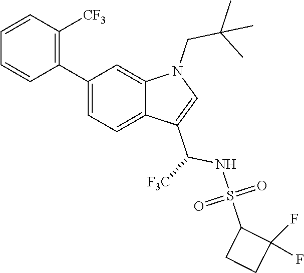 [SMILES: CC(C)(C)Cn1cc([C@H](NS(=O)(=O)C2CCC2(F)F)C(F)(F)F)c2ccc(-c3ccccc3C(F)(F)F)cc21] |
| 538 | 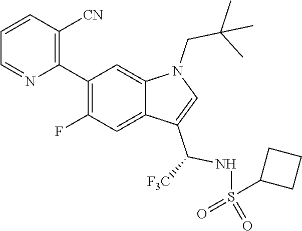 [SMILES: CC(C)(C)Cn1cc([C@H](NS(=O)(=O)C2CCC2)C(F)(F)F)c2cc(F)c(-c3ncccc3C#N)cc21] |
| 541 | 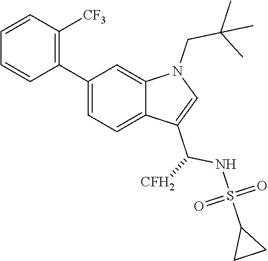 |
| 542 | 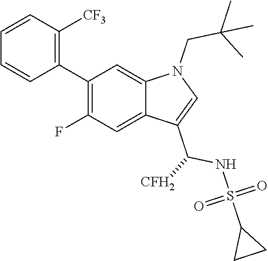 |
| 543 | 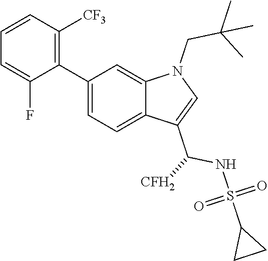 |
| 544 | 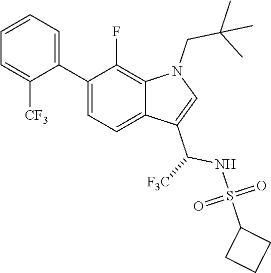 [SMILES: CC(C)(C)Cn1cc([C@H](NS(=O)(=O)C2CCC2)C(F)(F)F)c2ccc(-c3ccccc3C(F)(F)F)c(F)c21] |
| 545 | 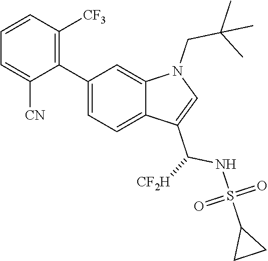 [SMILES: CC(C)(C)Cn1cc([C@H](NS(=O)(=O)C2CC2)C(F)F)c2ccc(-c3c(C#N)cccc3C(F)(F)F)cc21] |
| 546 | 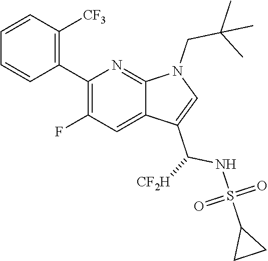 [SMILES: CC(C)(C)Cn1cc([C@H](NS(=O)(=O)C2CC2)C(F)F)c2cc(F)c(-c3ccccc3C(F)(F)F)nc21] |
| 547 | 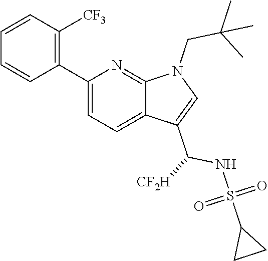 [SMILES: CC(C)(C)Cn1cc([C@H](NS(=O)(=O)C2CC2)C(F)F)c2ccc(-c3ccccc3C(F)(F)F)nc21] |
| 550 | 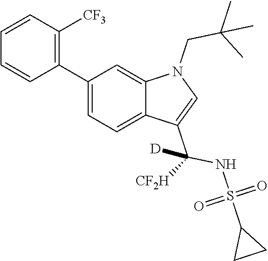 [SMILES: [2H][C@](NS(=O)(=O)C1CC1)(c1cn(CC(C)(C)C)c2cc(-c3ccccc3C(F)(F)F)ccc12)C(F)F] |
| 551 | 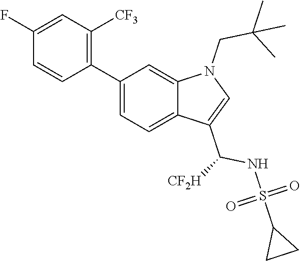 [SMILES: CC(C)(C)Cn1cc([C@H](NS(=O)(=O)C2CC2)C(F)F)c2ccc(-c3ccc(F)cc3C(F)(F)F)cc21] |
| 552 | 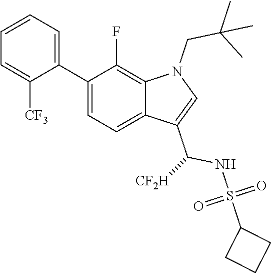 [SMILES: CC(C)(C)Cn1cc([C@H](NS(=O)(=O)C2CCC2)C(F)F)c2ccc(-c3ccccc3C(F)(F)F)c(F)c21] |
| 553 | 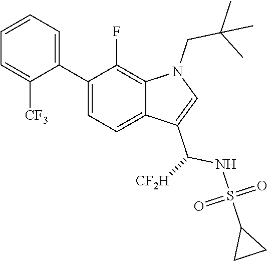 [SMILES: CC(C)(C)Cn1cc([C@H](NS(=O)(=O)C2CC2)C(F)F)c2ccc(-c3ccccc3C(F)(F)F)c(F)c21] |
| 555 | 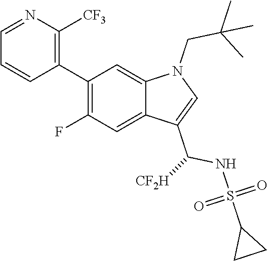 [SMILES: CC(C)(C)Cn1cc([C@H](NS(=O)(=O)C2CC2)C(F)F)c2cc(F)c(-c3cccnc3C(F)(F)F)cc21] |
| 557 | 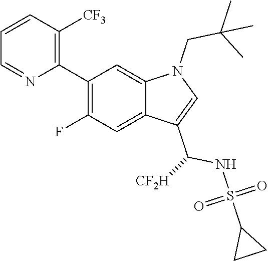 [SMILES: CC(C)(C)Cn1cc([C@H](NS(=O)(=O)C2CC2)C(F)F)c2cc(F)c(-c3ncccc3C(F)(F)F)cc21] |
| 558 | 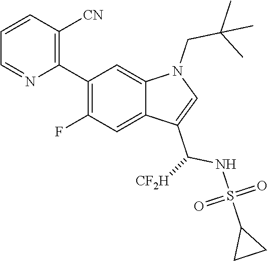 [SMILES: CC(C)(C)Cn1cc([C@H](NS(=O)(=O)C2CC2)C(F)F)c2cc(F)c(-c3ncccc3C#N)cc21] |
| 562 | 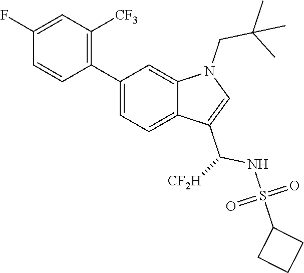 [SMILES: CC(C)(C)Cn1cc([C@H](NS(=O)(=O)C2CCC2)C(F)F)c2ccc(-c3ccc(F)cc3C(F)(F)F)cc21] |
| 563 | 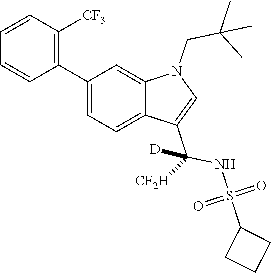 [SMILES: [2H][C@](NS(=O)(=O)C1CCC1)(c1cn(CC(C)(C)C)c2cc(-c3ccccc3C(F)(F)F)ccc12)C(F)F] |
| 564 | 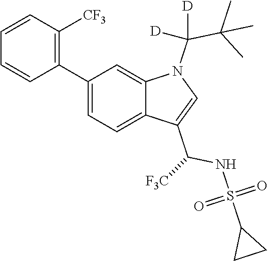 [SMILES: [2H]C([2H])(n1cc([C@H](NS(=O)(=O)C2CC2)C(F)(F)F)c2ccc(-c3ccccc3C(F)(F)F)cc21)C(C)(C)C] |
| 565 | 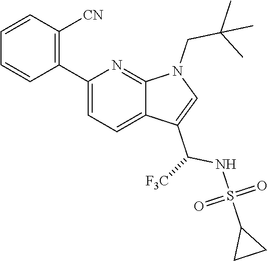 [SMILES: CC(C)(C)Cn1cc([C@H](NS(=O)(=O)C2CC2)C(F)(F)F)c2ccc(-c3ccccc3C#N)nc21] |
| 566 | 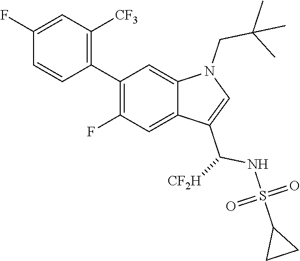 [SMILES: CC(C)(C)Cn1cc([C@H](NS(=O)(=O)C2CC2)C(F)F)c2cc(F)c(-c3ccc(F)cc3C(F)(F)F)cc21] |
| 567 | 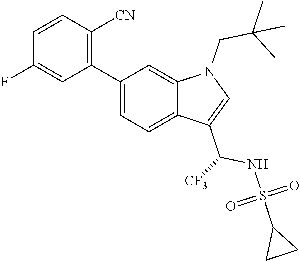 [SMILES: CC(C)(C)Cn1cc([C@H](NS(=O)(=O)C2CC2)C(F)(F)F)c2ccc(-c3cc(F)ccc3C#N)cc21] |
| 568 | 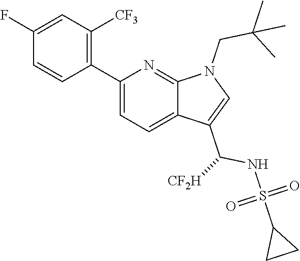 [SMILES: CC(C)(C)Cn1cc([C@H](NS(=O)(=O)C2CC2)C(F)F)c2ccc(-c3ccc(F)cc3C(F)(F)F)nc21] |

The present disclosure also provides in embodiments a pharmaceutical composition comprising a compound or a pharmaceutically acceptable salt or isotopologue thereof as described herein and a pharmaceutically acceptable carrier.

In an embodiment, the present disclosure provides a method for treating a subject suffering from hyperglycemia, dyslipidemia, atherosclerosis, autoimmune or inflammatory disorders or diseases, and cancers, such as glioblastoma, hepatocellular carcinoma, and colorectal cancer. The method comprises administering to the subject a therapeutically effective amount of a compound as described herein or a pharmaceutically acceptable salt thereof.

Still another embodiment of the present disclosure is a method for repressing T_{H}17 cell development in a subject. The method comprises administering to the subject a therapeutically effective amount of a compound as described herein or a pharmaceutically acceptable salt thereof.

The present disclosure, in an embodiment, provides a method for selectively agonizing REV-ERBα over REV-ERBβ in a subject. The method comprises administering to the subject a therapeutically effective amount of a compound as described herein or a pharmaceutically acceptable salt thereof.

The present disclosure also provides, in an embodiment, the use of a compound or pharmaceutically acceptable salt thereof as described herein for treating a subject suffering from hyperglycemia, dyslipidemia, atherosclerosis, an autoimmune or inflammatory disorder or disease, or a cancer, such as glioblastoma, hepatocellular carcinoma, and colorectal cancer.

The present disclosure also provides, in a further embodiment, the use of a compound or pharmaceutically acceptable salt thereof as described herein for repressing T_{H}17 cell development in a subject.

In an embodiment, the present disclosure provides a use of a compound or pharmaceutically acceptable salt thereof as described herein for selectively agonizing REV-ERBα over REV-ERBβ in a subject.

### DETAILED DESCRIPTION

The present disclosure provides in various embodiments small-molecule modulators of REV-ERB activity that are useful for treating T_{H}17-mediated autoimmune diseases or disorders. As described in various embodiments and illustrated by data, the small-molecule modulators are compounds that selectively agonize REV-ERBα relative to REV-ERBβ.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. A heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, the term “isotopologue” is an isotopically enriched compound. As used herein, and unless otherwise indicated, the term “isotopically enriched” refers to an atom having an isotopic composition other than the naturally abundant isotopic composition of that atom. “Isotopically enriched” can also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D,” it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound of Formula IA or Formula IB includes a pharmaceutically acceptable salt of a tautomer of the compound. Similarly, a compound of Formula IA or Formula IB includes a pharmaceutically acceptable salt of an isotopologue of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Compounds

As described in summary above, the present disclosure provides compounds according to Formula IA or IB, pharmaceutically acceptable salts, and/or tautomers and/or isotopologues thereof:

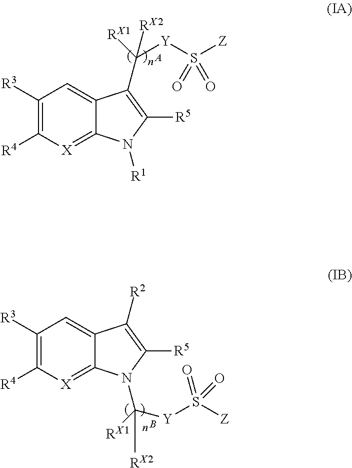

In some embodiments, the present disclosure provides Formula IA and IB compounds wherein X is CH, CCl, CF, CBr, C(C₁-C₆-alkyl), or N.

In these embodiments, R¹ is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S).

Further, R² is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —C(O)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S).

Any alkyl, alkoxy, aryl, cycloalkyl, and heteroaryl in R¹ and R² is optionally substituted with 1 to 6 substituents selected from the group consisting of halo, NO₂, OH, C₁-C₆-haloalkyl.

In addition, R^{X1} and R^{X2} are independently selected from the group consisting of H, C₁-C₆-alkyl, and C₁-C₆-haloalkyl,
or R^{X1} and R^{X2} together with the carbon atom to which they are bound form a spiro-fused C₃-C₅-cycloalkyl;
or any two vicinal R^{X1} and R^{X2} together with the carbon atoms to which they are bound form a C₃-C₅-cycloalkyl or a moiety selected from

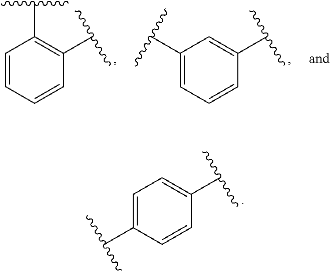 [SMILES: *c1ccccc1* ; *c1cccc(*)c1 ; *c1ccc(*)cc1]

In these embodiments, variable Y is —CR^{Y1}R^{Y2}— or —NR^{Y1}—; R^{Y1} and R^{Y2} are independently selected from H and C₁-C₆-alkyl; n^{A} is 0, 1, or 2; n^{B} is 1 or 2; and when Y is —NR^{Y1}—, then n^{A} is 1 or 2 and n^{B} is 2 and, optionally, R^{Y1} and one of R^{X1} and R^{X2}, together with the atoms to which they are bound, form a 3- to 6-membered heterocycloalkyl.

Also, variable Z is selected from the group consisting of C₁-C₆-alkyl, C₁-C₆-haloalkyl, C₃-C₅-cycloalkyl, 5- to 10-membered heteroaryl (wherein 1-4 heteroaryl members are independently selected from N, O, and S), 3- to 6-membered heterocycloalkyl (wherein 1-4 ring members are independently selected from N, O, and S), and —NR^{Z1}R^{Z2}, wherein R^{Z1} and R^{Z2} are independently selected from the group consisting of H, C₁-C₆-alkyl and C₃-C₈-cycloalkyl, or R^{Z1} and R^{Z2} together with the nitrogen to which they are bound form a 3- to 6-membered heterocycloalkyl. Any alkyl, aryl, cycloalkyl, heterocycloalkyl, and heteroaryl in Z is optionally substituted with 1 to 6 substituents selected from the group consisting of halo, NO₂, OH, and C₁-C₆-haloalkyl.

In addition, substituents R³ and R⁴ are independently selected from the group consisting of H, halo, CN, OH, N(R′)₂ (wherein each R′ is independently selected from H and C₁-C₆-alkyl), C₁-C₆-alkyl, C₁-C₆-haloalkyl, C₂-C₆-alkenyl, C₂-C₆-alkynyl, C₁-C₆-alkoxy, —O(C₁-C₆-alkyl)(C₆-C₁₀-aryl), C₃-C₈-cycloalkyl, C₆-C₁₀-aryl, and 5- to 10-membered heteroaryl (wherein 1-4 heteroaryl members are independently selected from N, O, and S), wherein R³ and R⁴ are not simultaneously H or simultaneously any combination of aryl, heteroaryl, and cycloalkyl; and wherein R³ and R⁴ are optionally substituted with 1 to 3 substituents selected from the group consisting of halo, NO₂, OH, C₁-C₆-haloalkyl, —C₁-C₆-haloalkyl(OH), —C(O)(C₁-C₆-alkyl), —C(O)O(C₁-C₆-alkyl).

Further, substituent R⁵ is selected from H and C₁-C₆-alkyl.

It should be understood that, notwithstanding the definitions provided herein, the compound of Formula IA or Formula IB does not include any of the following compounds:

| | |
|---|---|
| dimethyl({2-[3-(2-methylpropyl)-5-(pyridin-4-yl)- 1H-pyrrolo[2,3-b]pyridin-1- yl]ethyl}sulfamoyl)amine | 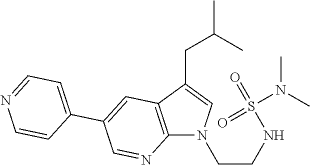 [SMILES: CC(C)Cc1cn(CCNS(=O)(=O)N(C)C)c2ncc(-c3ccncc3)cc12] |
| N-[2-(1,6-dimethyl-1H-indol-3-yl)ethyl]-3-methyl- 1-piperidinesulfonamide | 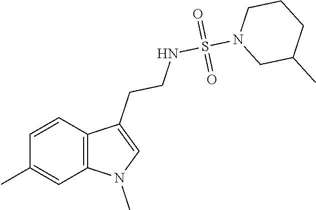 |
| N-[2-(1,6-dimethyl-1H-indol-3-yl)ethyl]-4-methyl- 1-piperidinesulfonamide | 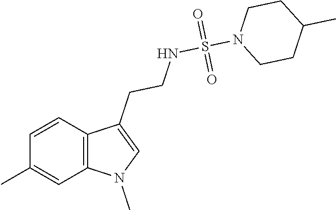 |
| N-[2-(1,6-dimethyl-1H-indol-3-yl)ethyl]-1- piperidinesulfonamide | 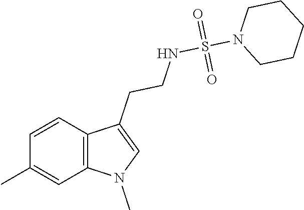 [SMILES: Cc1ccc2c(CCNS(=O)(=O)N3CCCCC3)cn(C)c2c1] |
| N-[2-(1,6-dimethyl-1H-indol-3-yl)ethyl]-1- pyrrolidinesulfonamide | 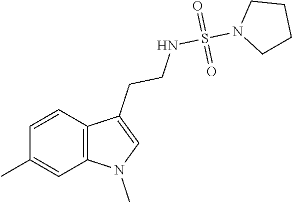 [SMILES: Cc1ccc2c(CCNS(=O)(=O)N3CCCC3)cn(C)c2c1] |
| N-[2-(1,6-dimethyl-1H-indol-3-yl)ethyl]-3-methyl- 1-pyrrolidinesulfonamide | 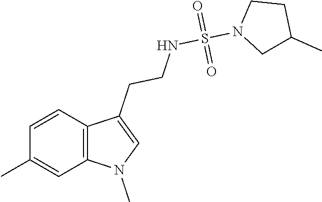 |
| N-[2-(1,6-dimethyl-1H-indol-3-yl)ethyl]-2-methyl- 1-piperidinesulfonamide | 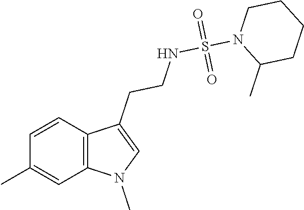 [SMILES: Cc1ccc2c(CCNS(=O)(=O)N3CCCCC3C)cn(C)c2c1] |
| N-[2-(1,6-dimethyl-1H-indol-3-yl)ethyl]-2- azabicyclo[2.2.1]heptane-2-sulfonamide | 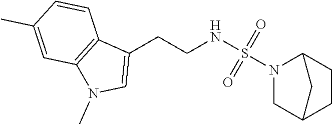 [SMILES: Cc1ccc2c(CCNS(=O)(=O)N3CC4CCC3C4)cn(C)c2c1] |
| N-[2-(1,6-dimethyl-1H-indol-3-yl)ethyl]-2-methyl- 1-pyrrolidinesulfonamide | 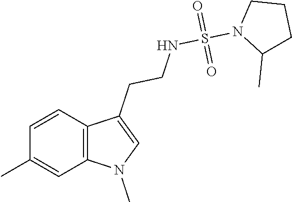 [SMILES: Cc1ccc2c(CCNS(=O)(=O)N3CCCC3C)cn(C)c2c1] |
| N-[2-(1,6-dimethyl-1H-indol-3-yl)ethyl]-4- morpholinesulfonamide | 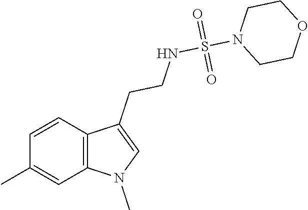 [SMILES: Cc1ccc2c(CCNS(=O)(=O)N3CCOCC3)cn(C)c2c1] |
| N-[2-(1,6-dimethyl-1H-indol-3-yl)ethyl]-2,6- dimethyl-4-morpholinesulfonamide | 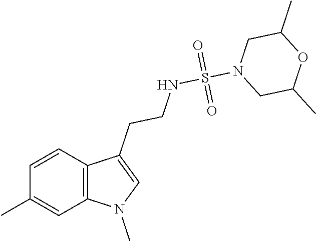 |
| N-[2-(1,6-dimethyl-1H-indol-3-yl)ethyl]-2,2- dimethyl-4-morpholinesulfonamide | 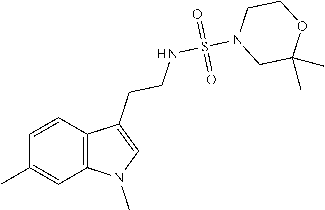 |
| 1-[[[2-(1,6-dimethyl-1H-indol-3- yl)ethyl]amino]sulfonyl]-4-Piperidinecarboxylic acid methyl ester | 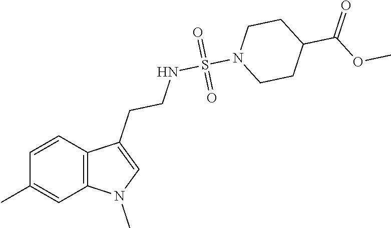 |

In various embodiments, X is CH in the compound of Formula IA or IB. In other embodiments, X is N.

In an embodiment, the compound is of Formula IA. In other embodiments, the compound is of Formula IB. Optionally in combination with any of these or any other embodiment described herein, R⁵ is H.

In combination with any other embodiment here, another embodiment provides for Y as —NR^{Y1}—.

In various embodiments optionally combinable with any other embodiment, n^{A} is 1 or 2. For example, n^{A} is 1. Alternatively, n^{A} is 2.

In some embodiments, R^{X1} and R^{X2}, when present, together with the carbon atom to which they are bound form a spiro-fused C₃-C₅-cycloalkyl. In other embodiments, any two vicinal R^{X1} and R^{X2} together with the carbon atoms to which they are bound form a C₃-C₅-cycloalkyl. An exemplary C₃-C₅-cycloalkyl is cyclopropyl.

In other embodiments, such as wherein n^{A} is 1, R^{X1} or R^{X2} is C₁-C₆-haloalkyl.

Optionally in combination with these embodiments is an embodiment providing for R^{X1} as H.

In possible combination with embodiments wherein R^{X1} and R^{X2} are present is an embodiment wherein R^{X1} is H and R^{X2} is —CF₃.

A further embodiment provides a Formula IA or Formula IB compound wherein Y is —NR^{Y1}—, n^{A} is 1 or 2 and n^{B} is 2. In addition, R^{Y1} and one of R^{X1} and R^{X2}, together with the atoms to which they are bound, form a 3- to 6-membered heterocycloalkyl. A non-limiting example of a 3- to 6-membered heterocycloalkyl is piperazinyl.

In various embodiments optionally in combination with any other embodiment herein described, R¹ and R² are independently selected from the group consisting of C₁-C₆-alkyl, C₁-C₆-haloalkyl, and —(C₁-C₆-alkyl)(C₃-C₈-cycloalkyl). For example, R¹ and R² are independently C₁-C₆-alkyl, such as C₄-C₆-alkyl. Examples of R¹ and R², per some embodiments, include iso-butyl and neo-pentyl.

In other embodiments optionally in combination with any other embodiment herein described, R¹ and R² are independently —(C₁-C₆-alkyl)(C₃-C₈-cycloalkyl).

Still further embodiments provide for any Formula IA or Formula IB compound described wherein Z is —NR^{Z1}R^{Z2}. Alternatively, per an embodiment, Z is C₁-C₆-alkyl or Z is C₃-C₅-cycloalkyl.

In various embodiments combinable with any other embodiment, R³ is H. Further, in accordance with another embodiment, R⁴ is H.

In an embodiment, the compound is of Formula IA. Further, X is CH; Y is —NR^{Y1}—; n^{A} is 1 or 2; R^{X1} and R^{X2} are independently selected from the group consisting of H, C₁-C₆-alkyl, and C₁-C₆-haloalkyl; R¹ is C₁-C₆-alkyl; Z is —NR^{Z1}R^{Z2} or C₁-C₆-alkyl; and R³ or R⁴ is H.

In accordance with various embodiments, the present disclosure also provides a compound of Formula IA, wherein X is CH or N; Y is —NR^{Y1}—; n^{A} is 1; R^{X1} is H; R² is C₁-C₆-alkyl or C₁-C₆-haloalkyl; R¹ is C₁-C₆-alkyl; Z is —C₃-C₅-cycloalkyl; R³ is halo; and R⁴ is optionally substituted C₆-C₁₀-aryl or 5- to 7-membered heteroaryl (wherein 1-4 heteroaryl members are independently selected from N, O, and S), and 3- to 6-membered heterocycloalkyl (wherein 1-4 ring members are independently selected from N, O, and S). In some of these embodiments, R^{Y1} is H; R² is selected from the group consisting of CH₃, CH₂F, CHF₂, and CF₃; R¹ is —CH₂^{t}Bu; and R⁴ is phenyl or pyridyl, wherein R⁴ is substituted with one or two substituents independently selected from the group consisting of F, Cl, CF₃, and CN.

In various additional embodiments, the present disclosure provides specific Formula IA and Formula IB compounds as shown in Tables 1A and 1B disclosed herein.

### Pharmaceutical Composition

The disclosure also provides a pharmaceutical composition comprising a therapeutically effective amount of one or more compounds according to Formula IA, Formula IB, or a pharmaceutically acceptable salt, stereoisomer, isotopologue, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in Table 1 or a pharmaceutically acceptable salt, stereoisomer, isotopologue, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, isotopologue, and/or tautomer thereof that is administered is governed by such considerations, and is the minimum amount necessary to repress T_{H}17 cell development, to exert an anti-inflammatory effect, to selectively agonize REV-ERBα over REV-ERBβ, or any combination thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of Formula IA or Formula IB may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Methods of Use

The compounds of the present disclosure are surprisingly selective for agonizing REV-ERBα over REV-ERBβ by a factor of at least about 3, 4, 5, 6, 7, 8, 9, or 10, which is the ratio [EC₅₀ (REV-ERBα)]/[EC₅₀ (REV-ERBβ)]. Thus, in an embodiment, the present disclosure contemplates a method for selectively agonizing REV-ERBα over REV-ERBβ in a subject, comprising administering to the subject a compound described herein or pharmaceutically acceptable salt thereof.

Further, the selectivity of the compounds for REV-ERBα underscores their usefulness in repressing T_{H}17 cell development. The present disclosure provides, in an embodiment, a method for repressing T_{H}17 in tissue in vitro, or in a subject in vivo, comprising contacting the tissue or administering to subject a compound described herein or pharmaceutically acceptable salt thereof.

This mechanism of action is operative, for example, in the treatment of T_{H}17-mediated autoimmunity and inflammation disorders, among others. Thus, the present disclosure also provides in various embodiments a method for treating a subject suffering from hyperglycemia, dyslipidemia, atherosclerosis, an autoimmune or inflammatory disorder or disease, or a cancer. Examples of cancers include glioblastoma, colorectal cancer, and hepatocellular carcinoma. The method comprises administering to the subject a therapeutically effective amount of a compound described herein or pharmaceutically acceptable salt thereof.

### EXAMPLES

The following non-limiting examples are additional embodiments for illustrating the present disclosure.

Compound Synthesis Examples: Part 1. Compound numbers in Part 1 are shown in parentheses in each example.

### Example 1: dimethyl({2-[1-(2-methylpropyl)-6-(pyridin-4-yl)-1H-indol-3-yl]ethyl}sulfamoyl)amine (27)

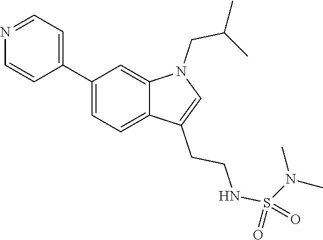 [SMILES: CC(C)Cn1cc(CCNS(=O)(=O)N(C)C)c2ccc(-c3ccncc3)cc21]

### Step 1. 6-bromo-1-isobutyl-1H-indole

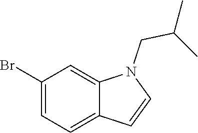

To 6-bromo-1H-indole (0.586 g, 2.99 mmol) in DMF (10 mL) was added Cs₂CO₃ (1.95 g, 6.0 mmol) and 1-bromo-2-methylpropane (0.65 mL, 6.0 mmol). The solution was stirred overnight in an 80° C. oil bath. The reaction was cooled to RT, diluted with EtOAc. The organic layer was washed with water, brine and dried over anhydrous Na₂SO₄. The organic layer was separated, and concentrated in vacuo. The crude product was purified by chromatography on silica gel (EtOAc/hex) to afford the title compound. ESI-MS (m/z): 251.86, 253.86 [M+1]⁺.

### Step 2. 2-(6-bromo-1-isobutyl-1H-indol-3-yl)-2-oxoacetamide

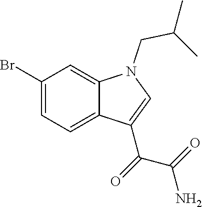 [SMILES: CC(C)Cn1cc(C(=O)C(N)=O)c2ccc(Br)cc21]

To the solution of 6-bromo-1-isobutyl-1H-indole (8.76 g, 34.74 mmol) in THF (100 mL) in an ice bath was slowly added oxalyl chloride (6 mL, 69.48 mml). The reaction was stirred 1h at RT. The solution was concentrated in vacuo. The crude was re-dissolved in fresh DCM and added slowly to cold ammonium hydroxide solution (50 mL, 25% in water, 347.4 mmol) in an ice bath. The solution was stirred for 30 min at RT. And then diluted with DCM. The aqueous layer was exacted with DCM twice. The organic layers were combined and washed with brine and dried over anhydrous Na₂SO₄. The solvent was removed in vacuo. to afford the title compound which was used in the next Step without further purification. ESI-MS (m/z): 322.87, 324.73 [M+1]⁺.

### Step 3. 2-(6-bromo-1-isobutyl-1H-indol-3-yl)ethan-1-amine

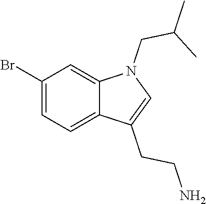 [SMILES: CC(C)Cn1cc(CCN)c2ccc(Br)cc21]

To the solution of 2-(6-bromo-1-isobutyl-1H-indol-3-yl)-2-oxoacetamide (1.7 g, 5.25 mmol) in THF (15 mL) was slowly added BH₃·DMS (2.0 mL, 20.99 mmol). The solution was then heated in an 70° C. oil bath for 2h. The reaction was monitored by reverse phase analytical HPLC. When the reaction was judged complete, the reaction solution was cooled to RT, and quenched with MeOH followed by the addition of 2N HCl until pH-3. The solution was refluxed for 2h and then the solvent was removed in vacuo to obtain the title compound with no further purification. ESI-MS (m/z): 294.95, 296.92 [M+1]⁺.

### Step 4. ({2-[6-bromo-1-(2-methylpropyl)-1H-indol-3-yl]ethyl}sulfamoyl)dimethylamine (97)

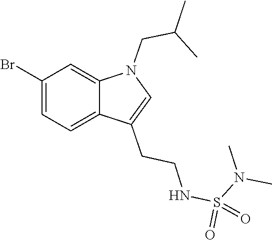 [SMILES: CC(C)Cn1cc(CCNS(=O)(=O)N(C)C)c2ccc(Br)cc21]

To the solution of 2-(6-bromo-1-isobutyl-1H-indol-3-yl)ethan-1-amine HCl salt (1.16 g, 3.49 mmol) in DCM (10 mL) was added TEA (1.5 mL, 10.48 mmol) followed by addition of dimethylsulfamoyl chloride (0.75 mL, 6.986 mmol). The solution was diluted with DCM, the organic layer was washed with Sat'd NaHCO₃, water and brine. The organic layer was separated and concentrated in vacuo. The crude product was purified by chromatography on silica gel (EtOAc/hex) to afford the title compound. ESI-MS (m/z): 402.03, 403.92 [M+1]⁺.

### Step 5: dimethyl({2-[1-(2-methylpropyl)-6-(pyridin-4-yl)-1H-indol-3-yl]ethyl}sulfamoyl)amine

A solution of ({2-[6-bromo-1-(2-methylpropyl)-1H-indol-3-yl]ethyl}sulfamoyl)dimethylamine (0.05 g, 0.124 mmol), pyridin-4-ylboronic acid (0.018, 0.149 mmol), K₂CO₃ (0.054 g, 0.372 mmol), and Pd(PPh₃)₄ (0.021 g, 0.019 mmol) in dioxane/water (4/1) (3 mL) was degassed and heated in a 80° C. oil bath for 16 h. The solvent was removed in vacuo to obtain the crude which was purified by prep-HPLC to yield the title compound as a TFA salt. ESI-MS (m/z): 401.12 [M+1]⁺.

### Example 2: dimethyl({2-[1-(2-methylpropyl)-6-(pyridin-3-yl)-1H-indol-3-yl]ethyl}sulfamoyl)amine (28)

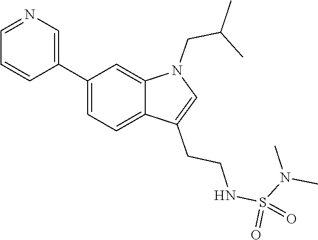 [SMILES: CC(C)Cn1cc(CCNS(=O)(=O)N(C)C)c2ccc(-c3cccnc3)cc21]

The title compound was prepared following the same general protocol as described for Step 5, Example 1, using pyridin-3-ylboronic acid. ESI-MS (m/z): 401.10 [M+1]⁺.

### Example 3: dimethyl({2-[1-(2-methylpropyl)-6-(pyrimidin-5-yl)-1H-indol-3-yl]ethyl}sulfamoyl)amine (29)

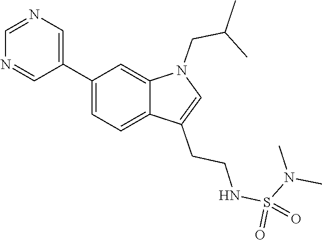 [SMILES: CC(C)Cn1cc(CCNS(=O)(=O)N(C)C)c2ccc(-c3cncnc3)cc21]

The title compound was prepared following the same general protocol as described for Step 5, Example 1, using pyrimidin-5-ylboronic acid. ESI-MS (m/z): 402.14 [M+1]⁺.

### Example 4: dimethyl({2-[1-(2-methylpropyl)-6-phenyl-1H-indol-3-yl]ethyl}sulfamoyl)amine (30)

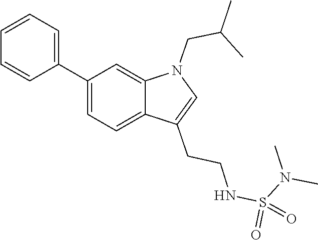 [SMILES: CC(C)Cn1cc(CCNS(=O)(=O)N(C)C)c2ccc(-c3ccccc3)cc21]

The title compound was prepared following the same general protocol as described for Step 5, Example 1, using phenylboronic acid. ESI-MS (m/z): 399.91 [M+1]⁺.

### Example 5: dimethyl({2-[1-(2-methylpropyl)-6-(pyridin-2-yl)-1H-indol-3-yl]ethyl}sulfamoyl)amine (31)

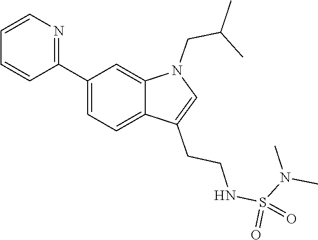 [SMILES: CC(C)Cn1cc(CCNS(=O)(=O)N(C)C)c2ccc(-c3ccccn3)cc21]

A solution of ({2-[6-bromo-1-(2-methylpropyl)-1H-indol-3-yl]ethyl}sulfamoyl)dimethylamine (0.06 g, 0.149 mmol), 2-(tributylstannyl)pyridine (0.066 g, 0.179 mmol), cesium fluoride (0.045 g, 0.298 mmol), CuI (0.003 g, 0.014 mmol) and Pd(PPh₃)₄ (0.017 g, 0.014 mmol) in DMF (1.0 mL) was degassed and heated in a 80° C. oil bath for 16 h. The solvent was removed in vacuo to obtain the crude which was purified by prep-HPLC to yield the title compound as a TFA salt. ESI-MS (m/z): 401.14 [M+1]⁺.

### Example 6: dimethyl({2-[1-(2-methylpropyl)-6-(pyrimidin-2-yl)-1H-indol-3-yl]ethyl}sulfamoyl)amine (32)

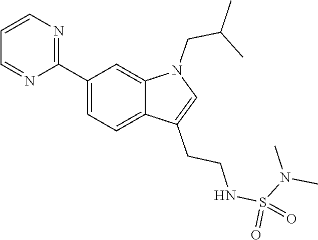 [SMILES: CC(C)Cn1cc(CCNS(=O)(=O)N(C)C)c2ccc(-c3ncccn3)cc21]

The title compound was prepared following the same general protocol as described for Example 5, using 2-(tributylstannyl)pyrimidine. ESI-MS (m/z): 402.07 [M+1]⁺.

### Example 7: dimethyl({2-[6-(1-methyl-1H-pyrazol-4-yl)-1-(2-methylpropyl)-1H-indol-3-yl]ethyl}sulfamoyl)amine (33)

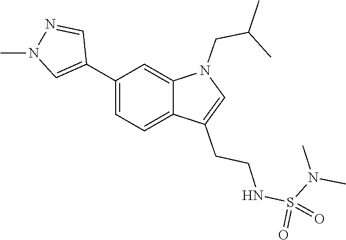

The title compound was prepared following the same general protocol as described for Step 5, Example 1, using 1-methyl-4-(4,4,5,5-tetramethyl-1,3,2-dioxaborolan-2-yl)-1H-pyrazole. ESI-MS (m/z): 404.16 [M+1]⁺.

### Example 8: dimethyl({2-[6-(1-methyl-1H-pyrazol-5-yl)-1-(2-methylpropyl)-1H-indol-3-yl]ethyl}sulfamoyl)amine (34)

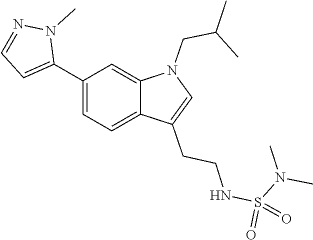 [SMILES: CC(C)Cn1cc(CCNS(=O)(=O)N(C)C)c2ccc(-c3ccnn3C)cc21]

The title compound was prepared following the same general protocol as described for Step 5, Example 1, using 1-methyl-5-(4,4,5,5-tetramethyl-1,3,2-dioxaborolan-2-yl)-1H-pyrazole. ESI-MS (m/z): 404.12 [M+1]⁺.

### Example 9: dimethyl({2-[1-(2-methylpropyl)-6-(1H-pyrazol-4-yl)-1H-indol-3-yl]ethyl}sulfamoyl)amine (35)

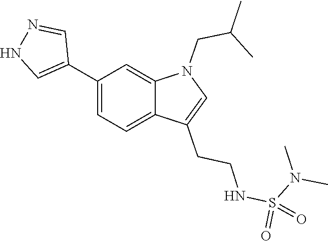 [SMILES: CC(C)Cn1cc(CCNS(=O)(=O)N(C)C)c2ccc(-c3cn[nH]c3)cc21]

The title compound was prepared following the same general protocol as described for Step 5, Example 1, using tert-butyl 4-(4,4,5,5-tetramethyl-1,3,2-dioxaborolan-2-yl)-1H-pyrazole-1-carboxylate. ESI-MS (m/z): 390.13 [M+1]⁺.

### Example 10: dimethyl({2-[1-(2-methylpropyl)-6-[2-(morpholin-4-yl)pyridin-4-yl]-1H-indol-3-yl]ethyl}sulfamoyl)amine (36)

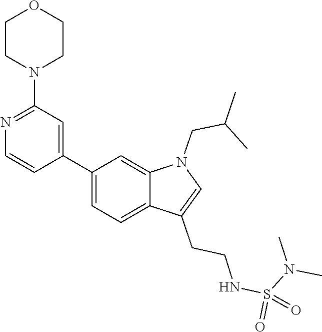 [SMILES: CC(C)Cn1cc(CCNS(=O)(=O)N(C)C)c2ccc(-c3ccnc(N4CCOCC4)c3)cc21]

The title compound was prepared following the same general protocol as described for Step 5, Example 1, using 4-(4-(4,4,5,5-tetramethyl-1,3,2-dioxaborolan-2-yl)pyridin-2-yl)morpholine. ESI-MS (m/z): 486.22 [M+1]⁺.

### Example 11: dimethyl({2-[1-(2-methylpropyl)-6-(quinolin-4-yl)-1H-indol-3-yl]ethyl}sulfamoyl)amine (37)

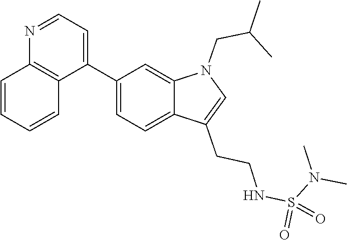 [SMILES: CC(C)Cn1cc(CCNS(=O)(=O)N(C)C)c2ccc(-c3ccnc4ccccc34)cc21]

The title compound was prepared following the same general protocol as described for Step 5, Example 1, using quinolin-4-ylboronic acid. ESI-MS (m/z): 451.18 [M+1]⁺.

### Example 12: ({2-[6-(isoquinolin-5-yl)-1-(2-methylpropyl)-1H-indol-3-yl]ethyl}sulfamoyl)dimethylamine (38)

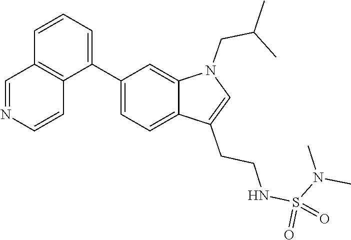 [SMILES: CC(C)Cn1cc(CCNS(=O)(=O)N(C)C)c2ccc(-c3cccc4cnccc34)cc21]

The title compound was prepared following the same general protocol as described for Step 5, Example 1, using isoquinolin-5-ylboronic acid. ESI-MS (m/z): 451.19 [M+1]⁺.

### Example 13: dimethyl({2-[1-(2-methylpropyl)-6-(naphthalen-1-yl)-1H-indol-3-yl]ethyl}sulfamoyl)amine (39)

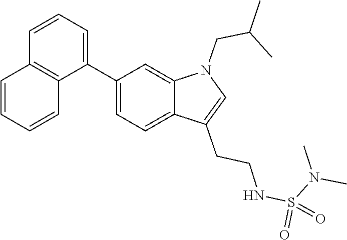 [SMILES: CC(C)Cn1cc(CCNS(=O)(=O)N(C)C)c2ccc(-c3cccc4ccccc34)cc21]

The title compound was prepared following the same general protocol as described for Step 5, Example 1, using naphthalen-1-ylboronic acid. ESI-MS (m/z): 449.95 [M+1]⁺.

### Example 14: ({2-[6-(3,5-dimethyl-1,2-oxazol-4-yl)-1-(2-methylpropyl)-1H-indol-3-yl]ethyl}sulfamoyl)dimethylamine (64)

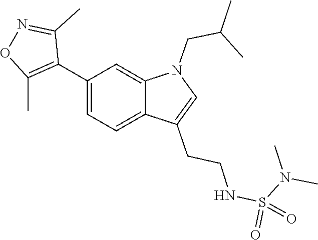 [SMILES: Cc1noc(C)c1-c1ccc2c(CCNS(=O)(=O)N(C)C)cn(CC(C)C)c2c1]

The title compound was prepared following the same general protocol as described for Step 5, Example 1, using 3,5-dimethyl-4-(4,4,5,5-tetramethyl-1,3,2-dioxaborolan-2-yl)isoxazole. ESI-MS (m/z): 418.91 [M+1]⁺.

### Example 15: dimethyl({2-[1-(2-methylpropyl)-6-(1,2-oxazol-4-yl)-1H-indol-3-yl]ethyl}sulfamoyl)amine (65)

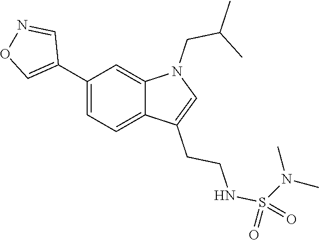 [SMILES: CC(C)Cn1cc(CCNS(=O)(=O)N(C)C)c2ccc(-c3cnoc3)cc21]

The title compound was prepared following the same general protocol as described for Step 5, Example 1, using 4-(4,4,5,5-tetramethyl-1,3,2-dioxaborolan-2-yl)isoxazole. ESI-MS (m/z): 390.82 [M+1]⁺.

### Example 16: ({2-[6-(furan-3-yl)-1-(2-methylpropyl)-1H-indol-3-yl]ethyl}sulfamoyl)dimethylamine (66)

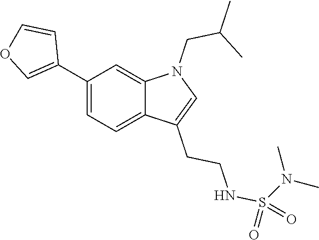 [SMILES: CC(C)Cn1cc(CCNS(=O)(=O)N(C)C)c2ccc(-c3ccoc3)cc21]

The title compound was prepared following the same general protocol as described for Step 5, Example 1, using 2-(furan-3-yl)-4,4,5,5-tetramethyl-1,3,2-dioxaborolane. ESI-MS (m/z): 389.89 [M+1]⁺.

### Example 17: dimethyl({2-[6-methyl-1-(2-methylpropyl)-1H-indol-3-yl]ethyl}sulfamoyl)amine (95)

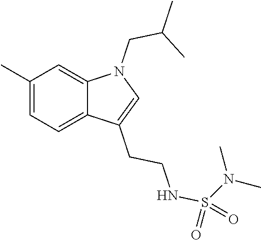

The title compound was prepared following the same general protocol as described for Step 5, Example 1, using 2,4,6-trimethyl-1,3,5,2,4,6-trioxatriborinane. ESI-MS (m/z): 337.89 [M+1]⁺.

### Example 18: dimethyl({2-[1-(2-methylpropyl)-6-(1,3-oxazol-2-yl)-1H-indol-3-yl]ethyl}sulfamoyl)amine (67) and dimethyl({2-[1-(2-methylpropyl)-1H-indol-3-yl]ethyl}sulfamoyl)amine (68)

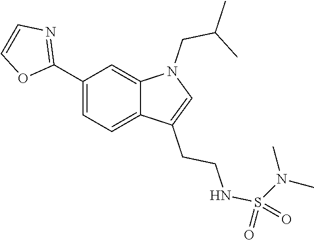 [SMILES: CC(C)Cn1cc(CCNS(=O)(=O)N(C)C)c2ccc(-c3ncco3)cc21]

The title compound (67) was prepared following the same general protocol as described for Example 5, using 2-(tributylstannyl)oxazole. ESI-MS (m/z): 390.99 [M+1]⁺.

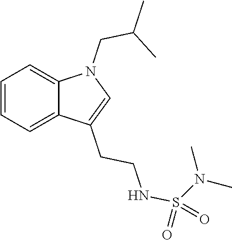 [SMILES: CC(C)Cn1cc(CCNS(=O)(=O)N(C)C)c2ccccc21]

The title compound (68) was prepared following the same general protocol as described for Example 5, using 2-(tributylstannyl)oxazole. ESI-MS (m/z): 323.87 [M+1]⁺.

### Example 19: dimethyl({2-[1-(2-methylpropyl)-6-(prop-1-en-2-yl)-1H-indol-3-yl]ethyl}sulfamoyl)amine (88)

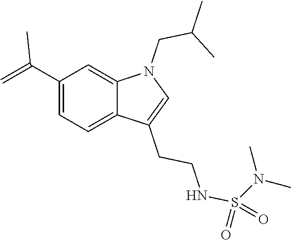 [SMILES: C=C(C)c1ccc2c(CCNS(=O)(=O)N(C)C)cn(CC(C)C)c2c1]

The title compound was prepared following the same general protocol as described for Step 5, Example 1, using 4,4,5,5-tetramethyl-2-(prop-1-en-2-yl)-1,3,2-dioxaborolane. ESI-MS (m/z): 363.86 [M+1]⁺.

### Example 20: dimethyl({2-[1-(2-methylpropyl)-6-(propan-2-yl)-1H-indol-3-yl]ethyl}sulfamoyl)amine (91)

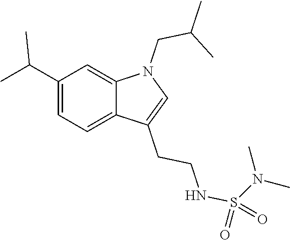 [SMILES: CC(C)Cn1cc(CCNS(=O)(=O)N(C)C)c2ccc(C(C)C)cc21]

To dimethyl({2-[1-(2-methylpropyl)-6-(prop-1-en-2-yl)-1H-indol-3-yl]ethyl}sulfamoyl)amine (0.04 g) in EtOH (3 mL) was added Pd/C (0.005 g). The solution was stirred under H₂ balloon overnight. The solution was filtered through a Celite Pad and washed with EtOAc. The filtrate was concentrated to obtain the title compound. ESI-MS (m/z): 365.85 [M+1]⁺.

### Example 21: dimethyl({2-[1-(2-methylpropyl)-6-propyl-1H-indol-3-yl]ethyl}sulfamoyl)amine (98)

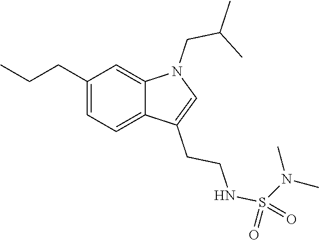 [SMILES: CCCc1ccc2c(CCNS(=O)(=O)N(C)C)cn(CC(C)C)c2c1]

### Step 1: dimethyl({2-[1-(2-methylpropyl)-6-[(1Z)-prop-1-en-1-yl]-1H-indol-3-yl]ethyl}sulfamoyl)amine

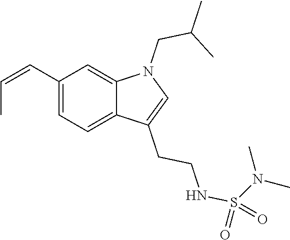 [SMILES: C/C=C\c1ccc2c(CCNS(=O)(=O)N(C)C)cn(CC(C)C)c2c1]

The title compound was prepared following the same general protocol as described for Step 5, Example 1, using (Z)-prop-1-en-1-ylboronic acid. ESI-MS (m/z): 363.89 [M+1]⁺.

### Step 2: dimethyl({2-[1-(2-methylpropyl)-6-propyl-1H-indol-3-yl]ethyl}sulfamoyl)amine

The title compound was prepared following the same general protocol as described for Example 20, using dimethyl({2-[1-(2-methylpropyl)-6-[(1Z)-prop-1-en-1-yl]-1H-indol-3-yl]ethyl}sulfamoyl)amine. ESI-MS (m/z): 365.92 [M+1]⁺.

### Example 22: ({2-[6-cyclopentyl-1-(2-methylpropyl)-1H-indol-3-yl]ethyl}sulfamoyl)dimethylamine (99)

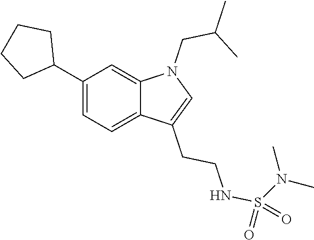 [SMILES: CC(C)Cn1cc(CCNS(=O)(=O)N(C)C)c2ccc(C3CCCC3)cc21]

### Step 1: ({2-[6-(cyclopent-1-en-1-yl)-1-(2-methylpropyl)-1H-indol-3-yl]ethyl}sulfamoyl)dimethylamine

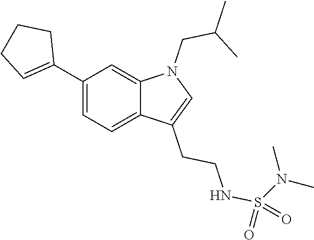 [SMILES: CC(C)Cn1cc(CCNS(=O)(=O)N(C)C)c2ccc(C3=CCCC3)cc21]

The title compound was prepared following the same general protocol as described for Step 5, Example 1, using 2-(cyclopent-1-en-1-yl)-4,4,5,5-tetramethyl-1,3,2-dioxaborolane. ESI-MS (m/z): 389.90 [M+1]⁺.

### Step 2: ({2-[6-cyclopentyl-1-(2-methylpropyl)-1H-indol-3-yl]ethyl}sulfamoyl)dimethylamine

The title compound was prepared following the same general protocol as described for Example 20, using ({2-[6-(cyclopent-1-en-1-yl)-1-(2-methylpropyl)-1H-indol-3-yl]ethyl}sulfamoyl)dimethylamine. ESI-MS (m/z): 391.91 [M+1]⁺.

### Example 23: ({2-[6-ethyl-1-(2-methylpropyl)-1H-indol-3-yl]ethyl}sulfamoyl)dimethylamine (100)

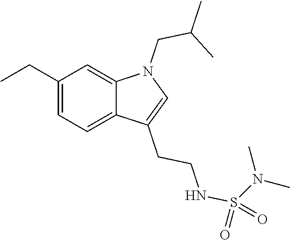 [SMILES: CCc1ccc2c(CCNS(=O)(=O)N(C)C)cn(CC(C)C)c2c1]

### Step 1: ({2-[6-ethenyl-1-(2-methylpropyl)-1H-indol-3-yl]ethyl}sulfamoyl)dimethylamine

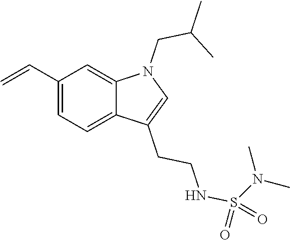 [SMILES: C=Cc1ccc2c(CCNS(=O)(=O)N(C)C)cn(CC(C)C)c2c1]

The title compound was prepared following the same general protocol as described for Example 5, using tributyl(vinyl)stannane. ESI-MS (m/z): 349.85 [M+1]⁺.

### Step 2: ({2-[6-cyclopentyl-1-(2-methylpropyl)-1H-indol-3-yl]ethyl}sulfamoyl)dimethylamine

The title compound was prepared following the same general protocol as described for Example 20, using ({2-[6-ethenyl-1-(2-methylpropyl)-1H-indol-3-yl]ethyl}sulfamoyl)dimethylamine. ESI-MS (m/z): 351.87 [M+1]⁺.

### Example 24: tert-butyl 4-(3-{2-[(dimethylsulfamoyl)amino]ethyl}-1-(2-methylpropyl)-1H-indol-6-yl)piperidine-1-carboxylate (101)

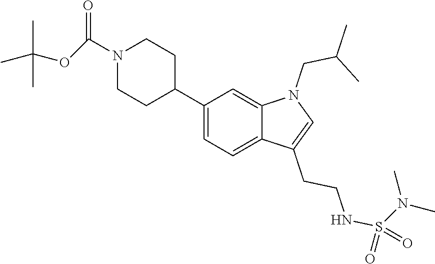 [SMILES: CC(C)Cn1cc(CCNS(=O)(=O)N(C)C)c2ccc(C3CCN(C(=O)OC(C)(C)C)CC3)cc21]

### Step 1: tert-butyl 4-(3-{2-[(dimethylsulfamoyl)amino]ethyl}-1-(2-methylpropyl)-1H-indol-6-yl)-1,2,3,6-tetrahydropyridine-1-carboxylate

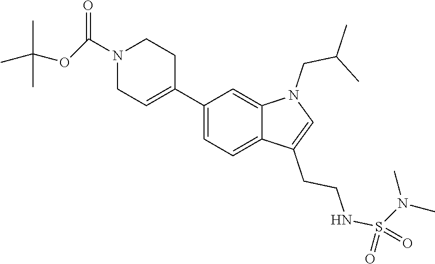 [SMILES: CC(C)Cn1cc(CCNS(=O)(=O)N(C)C)c2ccc(C3=CCN(C(=O)OC(C)(C)C)CC3)cc21]

The title compound was prepared following the same general protocol as described for Step 5, Example 1, using 1-(1-(tert-butoxy)vinyl)-4-(4,4,5,5-tetramethyl-1,3,2-dioxaborolan-2-yl)-1,2,3,6-tetrahydropyridine. ESI-MS (m/z): 504.99 [M+1]⁺.

### Step 2: ({2-[6-cyclopentyl-1-(2-methylpropyl)-1H-indol-3-yl]ethyl}sulfamoyl)dimethylamine

The title compound was prepared following the same general protocol as described for Example 20, using tert-butyl 4-(3-{2-[(dimethylsulfamoyl)amino]ethyl}-1-(2-methylpropyl)-1H-indol-6-yl)-1,2,3,6-tetrahydropyridine-1-carboxylate. ESI-MS (m/z): 506.55 [M+1]⁺.

### Example 25: dimethyl({2-[1-(2-methylpropyl)-6-(piperidin-4-yl)-1H-indol-3-yl]ethyl}sulfamoyl)amine (102)

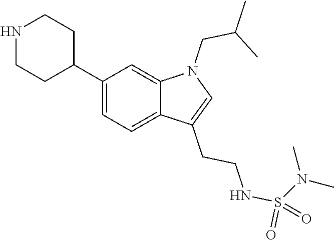 [SMILES: CC(C)Cn1cc(CCNS(=O)(=O)N(C)C)c2ccc(C3CCNCC3)cc21]

To the solution of tert-butyl 4-(3-{2-[(dimethylsulfamoyl)amino]ethyl}-1-(2-methylpropyl)-1H-indol-6-yl)piperidine-1-carboxylate (0.04 g) in DCM (0.5 mL) was added TFA (0.1 mL). The reaction was monitored by reverse phase analytical HPLC. When complete, the solvent was concentrated in vacuo to obtain the title compound as TFA salt. ESI-MS (m/z): 407.06 [M+1]⁺.

### Example 26: 1-[4-(3-{2-[(dimethylsulfamoyl)amino]ethyl}-1-(2-methylpropyl)-1H-indol-6-yl)piperidin-1-yl]ethan-1-one (103)

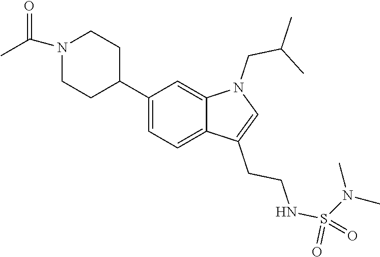 [SMILES: CC(=O)N1CCC(c2ccc3c(CCNS(=O)(=O)N(C)C)cn(CC(C)C)c3c2)CC1]

To the solution of dimethyl({2-[1-(2-methylpropyl)-6-(piperidin-4-yl)-1H-indol-3-yl]ethyl}sulfamoyl)amine (0.025 g, 0.061 mmol) in DCM (1 mL) was added NaHCO₃ (0.024 g, 0.31 mmol), followed by addition of acetyl chloride (1 drop). The reaction was monitored by reverse phase analytical HPLC. The solvent was concentrated in vacuo and the crude was purified by prep-HPLC to obtain the title compound as TFA salt. ESI-MS (m/z): 449.04 [M+1]⁺.

### Example 28: 3-{2-[(dimethylsulfamoyl)amino]ethyl}-1-(2-methylpropyl)-1H-indole-6-carbonitrile (96)

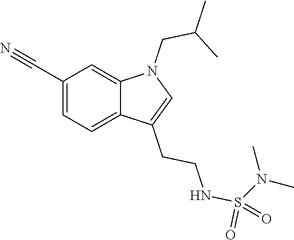 [SMILES: CC(C)Cn1cc(CCNS(=O)(=O)N(C)C)c2ccc(C#N)cc21]

A solution of ({2-[6-bromo-1-(2-methylpropyl)-1H-indol-3-yl]ethyl}sulfamoyl)dimethylamine (0.081 g, 0.2 mmol), Zn(CN)₂ (0.026 g, 0.22 mmol) and Pd(PPh₃)₄ (0.046 g, 0.02 mmol) in DMF (1 mL,) was degassed and heated in a 100° C. oil bath for 16 h. The solvent was removed in vacuo to obtain the crude which was purified by prep-HPLC to yield the title compound. ESI-MS (m/z): 348.81 [M+1]⁺.

### Example 29: 3-{2-[(dimethylsulfamoyl)amino]ethyl}-N-(1-methyl-1H-pyrazol-5-yl)-1-(2-methylpropyl)-1H-indol-6-amine (69)

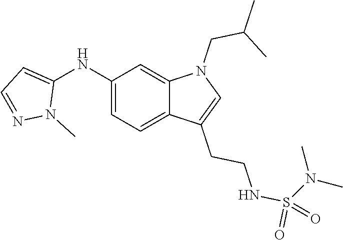 [SMILES: CC(C)Cn1cc(CCNS(=O)(=O)N(C)C)c2ccc(Nc3ccnn3C)cc21]

A solution of ({2-[6-bromo-1-(2-methylpropyl)-1H-indol-3-yl]ethyl}sulfamoyl)dimethylamine (0.03 g, 0.0746 mmol), 1-methyl-1H-pyrazol-5-amine (0.01 g, 0.104 mmol), NaOt-Bu (0.014 g, 0.149 mmol), Pd₂ (dba)₃ (0.005 g, 0.0075 mmol) and Xantphos (0.006 g, 0.022 mmol) in Toluene (1 mL) was degassed and heated in a 100° C. oil bath for 16 h. The solvent was removed in vacuo to obtain the crude which was purified by prep-HPLC to yield the title compound. ESI-MS (m/z): 419.09 [M+1]⁺.

### Example 30: N-(3-{2-[(dimethylsulfamoyl)amino]ethyl}-1-(2-methylpropyl)-1H-indol-6-yl)-2-phenylacetamide (70)

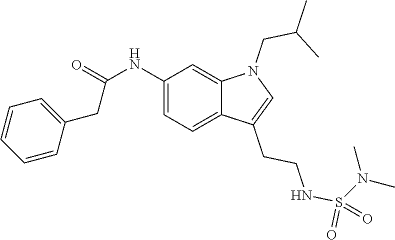 [SMILES: CC(C)Cn1cc(CCNS(=O)(=O)N(C)C)c2ccc(NC(=O)Cc3ccccc3)cc21]

### Step 1: [(2-{6-[(diphenylmethylidene)amino]-1-(2-methylpropyl)-1H-indol-3-yl}ethyl)sulfamoyl]dimethylamine

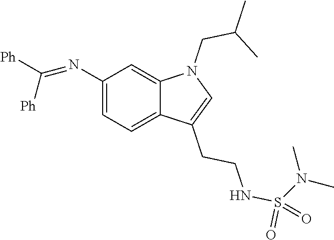 [SMILES: CC(C)Cn1cc(CCNS(=O)(=O)N(C)C)c2ccc(N=C(c3ccccc3)c3ccccc3)cc21]

A solution of ({2-[6-bromo-1-(2-methylpropyl)-1H-indol-3-yl]ethyl}sulfamoyl)dimethylamine (1.10 g, 2.73 mmol), diphenylmethanimine (0.595 g, 3.276 mmol), Cs₂CO₃ (1.78 g, 5.46 mmol), Pd₂ (dba)₃ (0.175 g, 0.191 mmol) and Xantphos (0.221 g, 0.382 mmol) in Toluene (12 mL) was degassed and heated in a 100° C. oil bath for 16 h. The solution was concentrated in vacuo. The crude product was purified by chromatography on silica gel (EtOAc/hex) to afford the title compound. ESI-MS (m/z): 503.20 [M+1]⁺.

### Step 2: 3-{2-[(dimethylsulfamoyl)amino]ethyl}-1-(2-methylpropyl)-1H-indol-6-amine

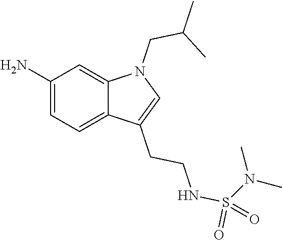 [SMILES: CC(C)Cn1cc(CCNS(=O)(=O)N(C)C)c2ccc(N)cc21]

To a solution of [(2-{6-[(diphenylmethylidene)amino]-1-(2-methylpropyl)-1H-indol-3-yl}ethyl)sulfamoyl]dimethylamine (1.29 g, 2.56 mmol) in THF (10 mL) at RT was added HCl in dioxane (1.3 mL, 4M, 5.12 mmol). The reaction was monitored by reverse phase analytical HPLC. The solution was diluted with hexanes and filtered to obtain the title compound as a solid. ESI-MS (m/z): 338.90 [M+1]⁺.

### Step 3: N-(3-{2-[(dimethylsulfamoyl)amino]ethyl}-1-(2-methylpropyl)-1H-indol-6-yl)-2-phenylacetamide

The title compound was prepared following the same general protocol as described for Example 26, using 3-{2-[(dimethylsulfamoyl)amino]ethyl}-1-(2-methylpropyl)-1H-indol-6-amine and 2-phenylacetyl chloride. ESI-MS (m/z): 456.97 [M+1]⁺.

### Example 31: N-(3-{2-[(dimethylsulfamoyl)amino]ethyl}-1-(2-methylpropyl)-1H-indol-6-yl)-2-phenylacetamide (71)

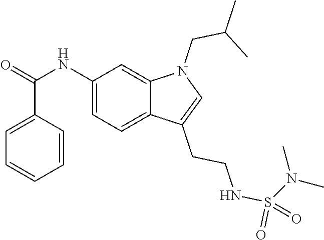 [SMILES: CC(C)Cn1cc(CCNS(=O)(=O)N(C)C)c2ccc(NC(=O)c3ccccc3)cc21]

The title compound was prepared following the same general protocol as described for Example 26, using 3-{2-[(dimethylsulfamoyl)amino]ethyl}-1-(2-methylpropyl)-1H-indol-6-amine and benzoyl chloride. ESI-MS (m/z): 442.94 [M+1]⁺.

### Example 33: dimethyl({2-[1-(2-methylpropyl)-6-[3-(trifluoromethyl)pyridin-4-yl]-1H-indol-3-yl]ethyl}sulfamoyl)amine (206)

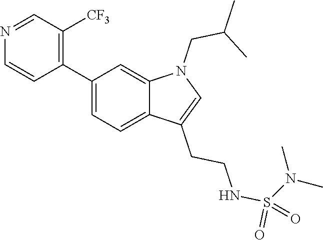 [SMILES: CC(C)Cn1cc(CCNS(=O)(=O)N(C)C)c2ccc(-c3ccncc3C(F)(F)F)cc21]

### Step 1: 3-(trifluoromethyl)pyridin-4-yl trifluoromethanesulfonate

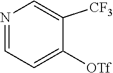 [SMILES: O=S(=O)(Oc1ccncc1C(F)(F)F)C(F)(F)F]

A solution of 3-(trifluoromethyl)pyridin-4-ol (1.0 g, 6.13 mmol), 1,1,1-trifluoro-N-phenyl-N-((trifluoromethyl)sulfonyl)methanesulfonamide (4.4 g, 12.26 mmol) and DIEA (4.3 mL, 24.52 mmol) in THF (20 mL) was stirred overnight at RT. The reaction was concentrated in vacuo to obtain the crude for next Step with no purification. ESI-MS (m/z): 295.82 [M+1]⁺.

### Step 2: dimethyl({2-[1-(2-methylpropyl)-6-(4,4,5,5-tetramethyl-1,3,2-dioxaborolan-2-yl)-1H-indol-3-yl]ethyl}sulfamoyl)amine

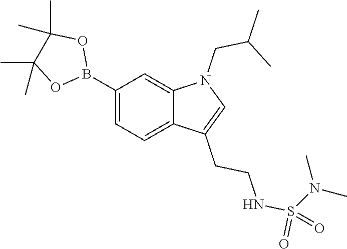

A solution of ({2-[6-bromo-1-(2-methylpropyl)-1H-indol-3-yl]ethyl}sulfamoyl)dimethylamine (0.2 g, 0.51 mmol), 4,4,4′,4′,5,5,5′,5′-octamethyl-2,2′-bi(1,3,2-dioxaborolane) (0.194 g, 0.756 mmol), KOAc (0.1 g, 1.01 mmol) and Pd(dppf)₂Cl₂ (0.042 g, 0.051 mmol) in dioxane (5 mL) was heated in a 100° C. oil bath for 16 h. The solution was concentrated in vacuo. The crude product was purified by chromatography on silica gel (EtOAc/hex) to afford the title compound. ESI-MS (m/z): 449.95 [M+1]⁺.

### Step 3: dimethyl({2-[1-(2-methylpropyl)-6-[3-(trifluoromethyl)pyridin-4-yl]-1H-indol-3-yl]ethyl}sulfamoyl)amine

The title compound was prepared following the same general protocol as described for Step 5, Example 1, using 3-(trifluoromethyl)pyridin-4-yl trifluoromethanesulfonate and dimethyl({2-[1-(2-methylpropyl)-6-(4,4,5,5-tetramethyl-1,3,2-dioxaborolan-2-yl)-1H-indol-3-yl]ethyl}sulfamoyl)amine. ESI-MS (m/z): 469.05 [M+1]⁺.

### Example 35: 3-{2-[(dimethylsulfamoyl)(methyl)amino]ethyl}-1-(2-methylpropyl)-1H-indole-6-carbonitrile (122)

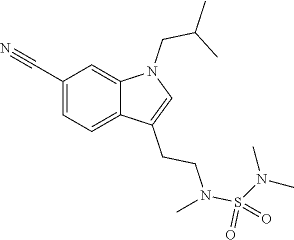 [SMILES: CC(C)Cn1cc(CCN(C)S(=O)(=O)N(C)C)c2ccc(C#N)cc21]

The title compound was prepared following the same general protocol as described for Example 34, using 3-{2-[(dimethylsulfamoyl)amino]ethyl}-1-(2-methylpropyl)-1H-indole-6-carbonitrile. ESI-MS (m/z): 362.76 [M+1]⁺.

### Example 36: ({2-[6-bromo-1-(2-methylpropyl)-1H-indol-3-yl]ethyl}(methyl)sulfamoyl)dimethylamine (123)

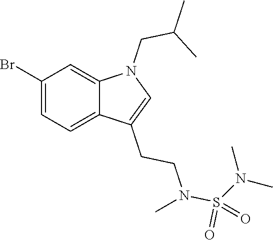 [SMILES: CC(C)Cn1cc(CCN(C)S(=O)(=O)N(C)C)c2ccc(Br)cc21]

The title compound was prepared following the same general protocol as described for Example 34, using ({2-[6-bromo-1-(2-methylpropyl)-1H-indol-3-yl]ethyl}sulfamoyl)dimethylamine. ESI-MS (m/z): 415.66, 417.55 [M+1]⁺.

### Example 37: dimethyl({2-[6-(pyridin-4-yl)-1H-indol-3-yl]ethyl}sulfamoyl)amine (40)

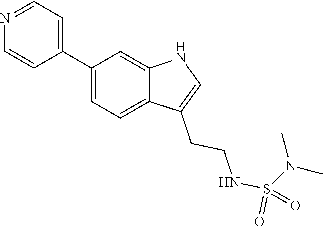 [SMILES: CN(C)S(=O)(=O)NCCc1c[nH]c2cc(-c3ccncc3)ccc12]

### Step 1: 2-(6-bromo-1H-indol-3-yl)-2-oxoacetic acid

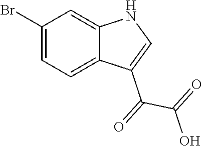 [SMILES: O=C(O)C(=O)c1c[nH]c2cc(Br)ccc12]

The title compound was prepared following the same general protocol as described for Step 2, Example 1, using 6-bromo-1H-indole and water. ESI-MS (m/z): 267.79, 269.72 [M+1]⁺.

### Step 2: 2-(6-bromo-1H-indol-3-yl)ethan-1-ol

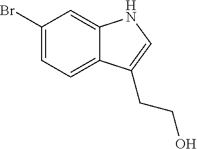 [SMILES: OCCc1c[nH]c2cc(Br)ccc12]

The title compound was prepared following the same general protocol as described for Step 3, Example 1, using 2-(6-bromo-1H-indol-3-yl)-2-oxoacetic acid at RT. ESI-MS (m/z): 239.71, 241.79 [M+1]⁺.

### Step 3: 6-bromo-3-(2-((tert-butyldimethylsilyl)oxy)ethyl)-1H-indole

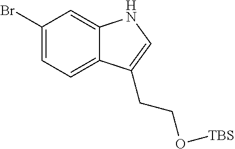

To a solution of 2-(6-bromo-1H-indol-3-yl)ethan-1-ol (6.187 g, 25.77 mmol) in DMF (50 mL) was added imidazole (3.5 g, 51.54 mmol), followed by addition of TBS-Cl (4.27 g, 28.347 mmol). The reaction was stirred at RT overnight. The solution was concentrated in vacuo. The crude product was dissolved in EtOAc, washed with water, brine, and dried (MgSO₄). The solvent was concentrated to obtain the crude which was purified by chromatography on silica gel (EtOAc/hex) to afford the title compound. ESI-MS (m/z): 355.02 [M+1]⁺.

### Step 4: tert-butyl 6-bromo-3-(2-((tert-butyldimethylsilyl)oxy)ethyl)-1H-indole-1-carboxylate

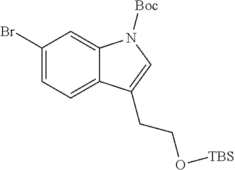

To a solution of 6-bromo-3-(2-((tert-butyldimethylsilyl)oxy)ethyl)-1H-indole (1.4 g, 3.95 mmol) in DCM (20 mL) was added TEA (1.1 mL, 7.9 mmol) and DMAP (0.1 g, 0.79 mmol), followed by addition of (Boc)₂O (1.73 g, 7.9 mmol). The reaction was stirred at RT overnight. The solution was concentrated in vacuo. The crude product was dissolved with EtOAc, washed with water and brine and dried (MgSO₄). The solvent was concentrated to obtain the crude which was purified by chromatography on silica gel (EtOAc/hex) to afford the title compound. ESI-MS (m/z): 455.02 [M+1]⁺.

### Step 5: tert-butyl 6-bromo-3-(2-hydroxyethyl)-1H-indole-1-carboxylate

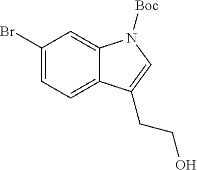 [SMILES: CC(C)(C)OC(=O)n1cc(CCO)c2ccc(Br)cc21]

To a solution of tert-butyl 6-bromo-3-(2-((tert-butyldimethylsilyl)oxy)ethyl)-1H-indole-1-carboxylate (1.91 g, 4.2 mmol) in THF (20 mL) at RT was added TBAF·3H₂O (2.0 g, 6.3 mmol). The reaction was monitored by reverse phase analytical HPLC. The solution was concentrated in vacuo. The crude product was dissolved with EtOAc, washed with water, brine and dried (MgSO₄). The solvent was concentrated to obtain the crude which was purified by chromatography on silica gel (EtOAc/hex) to afford the title compound. ESI-MS (m/z): 340.87 [M+1]⁺.

### Step 6: benzyl N-(dimethylsulfamoyl)carbamate

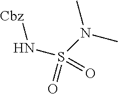 [SMILES: CN(C)S(=O)(=O)NC(=O)OCc1ccccc1]

To a solution of [(chlorosulfonyl)imino]methanone (4.2 mL, 48.317 mmol) in DCM (100 mL) in an ice bath was added dropwise BnOH (5.0 mL, 48.317 mmol). The reaction was stirred for 1h at RT and then transferred slowly to a solution of dimethylamine HCl salt (4.34 g, 53.149 mmol) and TEA (17 mL, 120.79 mmol) in DCM (50 mL) in an ice bath. The solution was stirred at RT overnight. The reaction was diluted with DCM, washed with water, brine and dried over anhydrous Na₂SO₄. The organic layer was separated by filtration and concentrated to obtain the title compound with no further purification.

### Step 7: tert-butyl 3-(2-{[(benzyloxy)carbonyl](dimethylsulfamoyl)amino}ethyl)-6-bromo-1H-indole-1-carboxylate

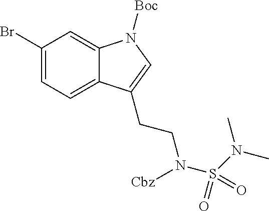 [SMILES: CN(C)S(=O)(=O)N(CCc1cn(C(=O)OC(C)(C)C)c2cc(Br)ccc12)C(=O)OCc1ccccc1]

To a solution of benzyl N-(dimethylsulfamoyl)carbamate (0.73 g, 2.82 mmol) in THF (10 mL) in an ice bath was added PPh₃ (1.48 g, 5.64 mmol) and tert-butyl 6-bromo-3-(2-hydroxyethyl)-1H-indole-1-carboxylate (0.96 g, 2.82 mmol), followed by slow addition of DIAD (1.1 mL, 5.64 mmol). The reaction was then stirred at RT overnight. The solvent was concentrated to obtain the crude which was purified by chromatography on silica gel (EtOAc/hex) to afford the title compound. ESI-MS (m/z): 579.78, 581.74 [M+1]⁺.

### Step 8: tert-butyl 3-{2-[(dimethylsulfamoyl)amino]ethyl}-6-(pyridin-4-yl)-1H-indole-1-carboxylate

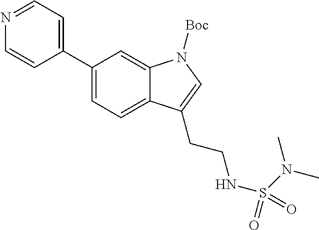 [SMILES: CN(C)S(=O)(=O)NCCc1cn(C(=O)OC(C)(C)C)c2cc(-c3ccncc3)ccc12]

The title compound was prepared following the same general protocol as described for Step 5, Example 1, using tert-butyl 3-(2-{[(benzyloxy)carbonyl](dimethylsulfamoyl)amino}ethyl)-6-bromo-1H-indole-1-carboxylate. ESI-MS (m/z): 445.10[M+1]⁺.

### Step 9: dimethyl({2-[6-(pyridin-4-yl)-1H-indol-3-yl]ethyl}sulfamoyl)amine

The title compound was prepared following the same general protocol as described for Example 25, using tert-butyl 3-{2-[(dimethylsulfamoyl)amino]ethyl}-6-(pyridin-4-yl)-1H-indole-1-carboxylate. ESI-MS (m/z): 345.09 [M+1]⁺.

### Example 38: 1-(3-{2-[(dimethylsulfamoyl)amino]ethyl}-6-(pyridin-4-yl)-1H-indol-1-yl)ethan-1-one (42)

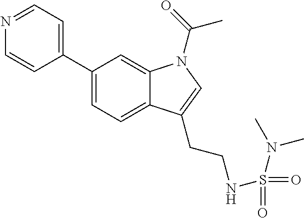 [SMILES: CC(=O)n1cc(CCNS(=O)(=O)N(C)C)c2ccc(-c3ccncc3)cc21]

To a solution of dimethyl({2-[6-(pyridin-4-yl)-1H-indol-3-yl]ethyl}sulfamoyl)amine hydrochloride (0.04 g, 0.105 mmol) in DCM (1 mL) was added tetrabutylammonium hydrosulfate (0.007 g, 0.021 mmol) and NaOH (0.015 g, 0.368 mmol), followed by addition of acetyl chloride (0.02 g, 0.263 mmol) in DCM (0.1 mL). The solution was stirred at RT for 4h. After completion, the reaction was diluted with DCM, and washed with water and brine. The organic layer was concentrated to obtain the crude which was purified by prep-HPLC to afford the title compound. ESI-MS (m/z): 387.03 [M+1]⁺.

### Example 39: 1-(3-{2-[(dimethylsulfamoyl)amino]ethyl}-6-(pyridin-4-yl)-1H-indol-1-yl)-2-methylpropan-1-one (43)

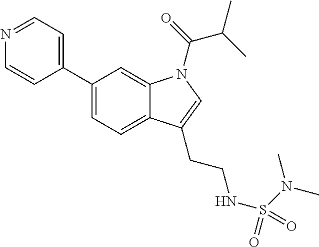 [SMILES: CC(C)C(=O)n1cc(CCNS(=O)(=O)N(C)C)c2ccc(-c3ccncc3)cc21]

The title compound was prepared following the same general protocol as described for Example 38, using isobutyryl chloride. ESI-MS (m/z): 415.07[M+1]⁺.

### Example 40: 1-(3-{2-[(dimethylsulfamoyl)amino]ethyl}-6-(pyridin-4-yl)-1H-indol-1-yl)-3-methylbutan-1-one (155)

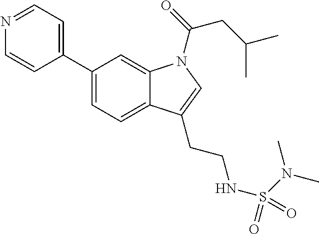 [SMILES: CC(C)CC(=O)n1cc(CCNS(=O)(=O)N(C)C)c2ccc(-c3ccncc3)cc21]

The title compound was prepared following the same general protocol as described for Example 38, using 3-methylbutanoyl chloride. ESI-MS (m/z): 428.97 [M+1]⁺.

### Example 41: 1-(3-{2-[(dimethylsulfamoyl)amino]ethyl}-6-(pyridin-4-yl)-1H-indol-1-yl)-2,2dimethylpropan-1-one (156)

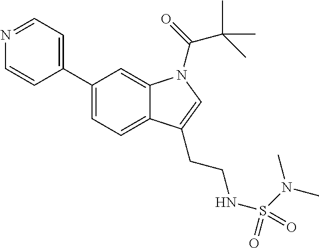 [SMILES: CN(C)S(=O)(=O)NCCc1cn(C(=O)C(C)(C)C)c2cc(-c3ccncc3)ccc12]

The title compound was prepared following the same general protocol as described for Example 38, using pivaloyl chloride. ESI-MS (m/z): 429.00 [M+1]⁺.

### Example 42: {[2-(6-bromo-1-ethyl-1H-indol-3-yl)ethyl]sulfamoyl}dimethylamine (157)

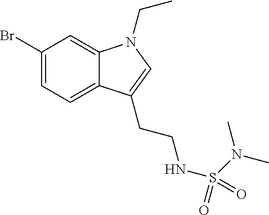 [SMILES: CCn1cc(CCNS(=O)(=O)N(C)C)c2ccc(Br)cc21]

### Step 1: tert-butyl (N,N-dimethylsulfamoyl)carbamate

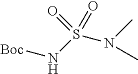

The title compound was prepared following the same general protocol as described for Step 6, Example 37, using tert-butyl alcohol. ¹H-NMR (CDCl₃, 400 MHz) δ 7.05 (broad s, 1H), 2.95 (s, 6H), 1.50 (s, 9H)

### Step 2: tert-butyl (2-(6-bromo-1H-indol-3-yl)ethyl)(N,N-dimethylsulfamoyl)carbamate

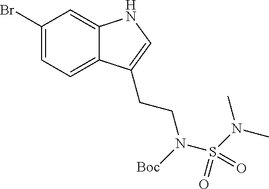 [SMILES: CN(C)S(=O)(=O)N(CCc1c[nH]c2cc(Br)ccc12)C(=O)OC(C)(C)C]

The title compound was prepared following the same general protocol as described for Step 7, Example 37, using tert-butyl (N,N-dimethylsulfamoyl)carbamate and 2-(6-bromo-1H-indol-3-yl)ethan-1-ol. ESI-MS (m/z): 445.75, 447.76 [M+1]⁺.

### Step 3: tert-butyl (2-(6-bromo-1-ethyl-1H-indol-3-yl)ethyl)(N,N-dimethylsulfamoyl)carbamate

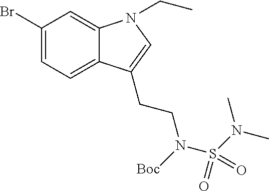 [SMILES: CCn1cc(CCN(C(=O)OC(C)(C)C)S(=O)(=O)N(C)C)c2ccc(Br)cc21]

The title compound was prepared following the same general protocol as described for Example 34, using tert-butyl (2-(6-bromo-1H-indol-3-yl)ethyl)(N,N-dimethylsulfamoyl)carbamate and ethyl iodide. ESI-MS (m/z): 473.65, 475.70 [M+1]⁺.

### Step 4: {[2-(6-bromo-1-ethyl-1H-indol-3-yl)ethyl]sulfamoyl}dimethylamine

The title compound was prepared following the same general protocol as described for Example 25, using tert-butyl (2-(6-bromo-1-ethyl-1H-indol-3-yl)ethyl)(N,N-dimethylsulfamoyl)carbamate. ESI-MS (m/z): 373.63, 375.70 [M+1]⁺.

### Example 43: ({2-[6-bromo-1-(2,2,2-trifluoroethyl)-1H-indol-3-yl]ethyl}sulfamoyl)dimethylamine (158)

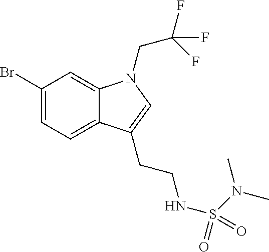 [SMILES: CN(C)S(=O)(=O)NCCc1cn(CC(F)(F)F)c2cc(Br)ccc12]

### Step 1: tert-butyl (2-(6-bromo-1-(2,2,2-trifluoroethyl)-1H-indol-3-yl)ethyl)(N,N-dimethylsulfamoyl)carbamate

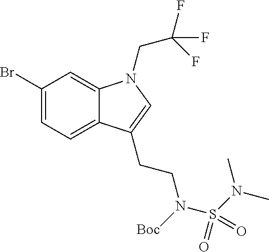 [SMILES: CN(C)S(=O)(=O)N(CCc1cn(CC(F)(F)F)c2cc(Br)ccc12)C(=O)OC(C)(C)C]

The title compound was prepared following the same general protocol as described for Example 34, using tert-butyl (2-(6-bromo-1H-indol-3-yl)ethyl)(N,N-dimethylsulfamoyl)carbamate and 2,2,2-trifluoroethyl trifluoromethanesulfonate. ESI-MS (m/z): 527.80, 529.85 [M+1]⁺.

### Step 2: ({2-[6-bromo-1-(2,2,2-trifluoroethyl)-1H-indol-3-yl]ethyl}sulfamoyl)dimethylamine

The title compound was prepared following the same general protocol as described for Example 25, using tert-butyl (2-(6-bromo-1-(2,2,2-trifluoroethyl)-1H-indol-3-yl)ethyl)(N,N-dimethylsulfamoyl)carbamate. ESI-MS (m/z): 427.69, 429.71 [M+1]⁺.

### Example 44: ({2-[6-bromo-1-(1-phenylethyl)-1H-indol-3-yl]ethyl}sulfamoyl)dimethylamine (159)

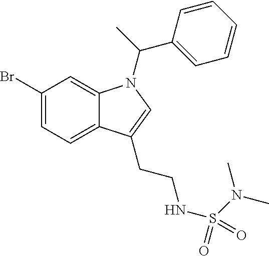 [SMILES: CC(c1ccccc1)n1cc(CCNS(=O)(=O)N(C)C)c2ccc(Br)cc21]

### Step 1: tert-butyl (2-(6-bromo-1-(1-phenylethyl)-1H-indol-3-yl)ethyl) (N,N-imethylsulfamoyl)carbamate

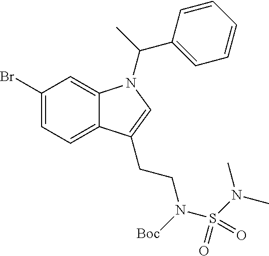 [SMILES: CC(c1ccccc1)n1cc(CCN(C(=O)OC(C)(C)C)S(=O)(=O)N(C)C)c2ccc(Br)cc21]

The title compound was prepared following the same general protocol as described for Example 34, using tert-butyl (2-(6-bromo-1H-indol-3-yl)ethyl)(N,N-dimethylsulfamoyl)carbamate and (1-bromoethyl)benzene. ESI-MS (m/z): 549.68, 551.78 [M+1]⁺.

### Step 2: ({2-[6-bromo-1-(1-phenylethyl)-1H-indol-3-yl]ethyl}sulfamoyl)dimethylamine

The title compound was prepared following the same general protocol as described for Example 25, using tert-butyl (2-(6-bromo-1-(1-phenylethyl)-1H-indol-3-yl)ethyl)(N,N-dimethylsulfamoyl)carbamate. ESI-MS (m/z): 449.63, 451.66 [M+1]⁺.

### Example 45: [2-(6-bromo-3-{2-[(dimethylsulfamoyl)amino]ethyl}-1H-indol-1-yl)ethyl]dimethylamine (160)

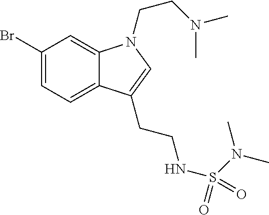 [SMILES: CN(C)CCn1cc(CCNS(=O)(=O)N(C)C)c2ccc(Br)cc21]

### Step 1: tert-butyl (2-(6-bromo-1-(2-(dimethylamino)ethyl)-1H-indol-3-yl)ethyl)(N,N-dimethylsulfamoyl)carbamate

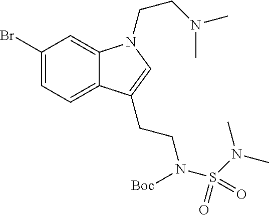 [SMILES: CN(C)CCn1cc(CCN(C(=O)OC(C)(C)C)S(=O)(=O)N(C)C)c2ccc(Br)cc21]

The title compound was prepared following the same general protocol as described for Example 34, using tert-butyl (2-(6-bromo-1H-indol-3-yl)ethyl)(N,N-dimethylsulfamoyl)carbamate and 2-chloro-N,N-dimethylethan-1-amine HCl. ESI-MS (m/z): 516.86, 518.83 [M+1]⁺.

### Step 2: [2-(6-bromo-3-{2-[(dimethylsulfamoyl)amino]ethyl}-1H-indol-1-yl)ethyl]dimethylamine

The title compound was prepared following the same general protocol as described for Example 25, using tert-butyl (2-(6-bromo-1-(2-(dimethylamino)ethyl)-1H-indol-3-yl)ethyl)(N,N-dimethylsulfamoyl)carbamate. ESI-MS (m/z): 416.84, 418.83 [M+1]⁺.

### Example 46: [(2-{6-bromo-1-[(pyridin-2-yl)methyl]-1H-indol-3-yl}ethyl)sulfamoyl]dimethylamine (161)

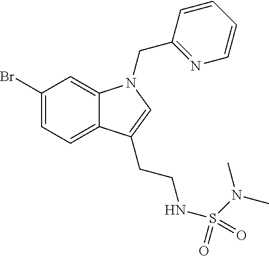 [SMILES: CN(C)S(=O)(=O)NCCc1cn(Cc2ccccn2)c2cc(Br)ccc12]

### Step 1: tert-butyl (2-(6-bromo-1-(pyridin-2-ylmethyl)-1H-indol-3-yl)ethyl)(N,N-dimethylsulfamoyl)carbamate

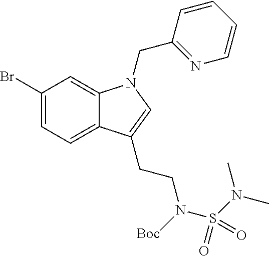 [SMILES: CN(C)S(=O)(=O)N(CCc1cn(Cc2ccccn2)c2cc(Br)ccc12)C(=O)OC(C)(C)C]

The title compound was prepared following the same general protocol as described for Example 34, using tert-butyl (2-(6-bromo-1H-indol-3-yl)ethyl)(N,N-dimethylsulfamoyl)carbamate and 2-(bromomethyl)pyridine HBr. ESI-MS (m/z): 536.75, 538.78 [M+1]⁺.

### Step 2: [(2-{6-bromo-1-[(pyridin-2-yl)methyl]-1H-indol-3-yl}ethyl)sulfamoyl]dimethylamine

The title compound was prepared following the same general protocol as described for Example 25, using tert-butyl (2-(6-bromo-1-(pyridin-2-ylmethyl)-1H-indol-3-yl)ethyl)(N,N-dimethylsulfamoyl)carbamate. ESI-MS (m/z): 436.77, 438.74 [M+1]⁺.

### Example 47: [(2-{6-bromo-1-[(pyridin-4-yl)methyl]-1H-indol-3-yl}ethyl)sulfamoyl]dimethylamine (162)

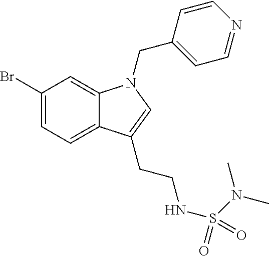 [SMILES: CN(C)S(=O)(=O)NCCc1cn(Cc2ccncc2)c2cc(Br)ccc12]

### Step 1: tert-butyl (2-(6-bromo-1-(pyridin-4-ylmethyl)-1H-indol-3-yl)ethyl)(N,N-dimethylsulfamoyl)carbamate

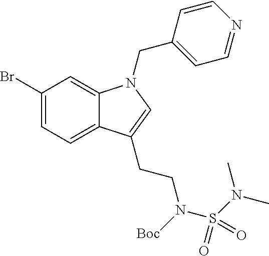 [SMILES: CN(C)S(=O)(=O)N(CCc1cn(Cc2ccncc2)c2cc(Br)ccc12)C(=O)OC(C)(C)C]

The title compound was prepared following the same general protocol as described for Example 34, using tert-butyl (2-(6-bromo-1H-indol-3-yl)ethyl)(N,N-dimethylsulfamoyl)carbamate and 4-(bromomethyl)pyridine HBr. ESI-MS (m/z): 536.84, 538.79 [M+1]⁺.

### Step 2: [(2-{6-bromo-1-[(pyridin-4-yl)methyl]-1H-indol-3-yl}ethyl)sulfamoyl]dimethylamine

The title compound was prepared following the same general protocol as described for Example 25, using tert-butyl (2-(6-bromo-1-(pyridin-4-ylmethyl)-1H-indol-3-yl)ethyl)(N,N-dimethylsulfamoyl)carbamate. ESI-MS (m/z): 436.88, 438.87 [M+1]⁺.

### Example 48: [(2-{6-bromo-1-[(pyridin-3-yl)methyl]-1H-indol-3-yl}ethyl)sulfamoyl]dimethylamine (163)

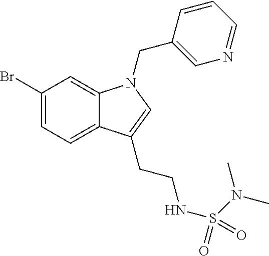 [SMILES: CN(C)S(=O)(=O)NCCc1cn(Cc2cccnc2)c2cc(Br)ccc12]

### Step 1: tert-butyl (2-(6-bromo-1-(pyridin-3-ylmethyl)-1H-indol-3-yl)ethyl)(N,N-dimethylsulfamoyl)carbamate

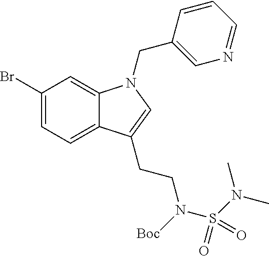 [SMILES: CN(C)S(=O)(=O)N(CCc1cn(Cc2cccnc2)c2cc(Br)ccc12)C(=O)OC(C)(C)C]

The title compound was prepared following the same general protocol as described for Example 34, using tert-butyl (2-(6-bromo-1H-indol-3-yl)ethyl)(N,N-dimethylsulfamoyl)carbamate and 3-(bromomethyl)pyridine HBr. ESI-MS (m/z): 536.74, 538.78 [M+1]⁺.

### Step 2: [(2-{6-bromo-1-[(pyridin-3-yl)methyl]-1H-indol-3-yl}ethyl)sulfamoyl]dimethylamine

The title compound was prepared following the same general protocol as described for Example 25, using tert-butyl (2-(6-bromo-1-(pyridin-3-ylmethyl)-1H-indol-3-yl)ethyl)(N,N-dimethylsulfamoyl)carbamate. ESI-MS (m/z): 436.77, 438.79 [M+1]⁺.

### Example 49: methyl 2-(6-bromo-3-(2-((N,N-dimethylsulfamoyl)amino)ethyl)-1H-indol-1-yl)acetate (164)

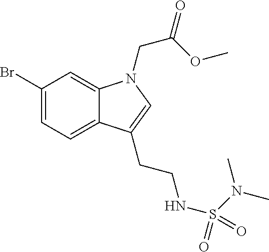 [SMILES: COC(=O)Cn1cc(CCNS(=O)(=O)N(C)C)c2ccc(Br)cc21]

### Step 1: methyl 2-(6-bromo-3-(2-((tert-butoxycarbonyl)(N,N-dimethylsulfamoyl)amino)ethyl)-1H-indol-1-yl)acetate

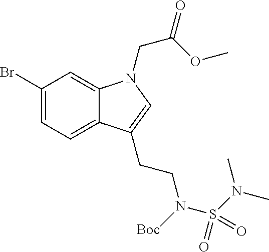 [SMILES: COC(=O)Cn1cc(CCN(C(=O)OC(C)(C)C)S(=O)(=O)N(C)C)c2ccc(Br)cc21]

The title compound was prepared following the same general protocol as described for Example 34, using tert-butyl (2-(6-bromo-1H-indol-3-yl)ethyl)(N,N-dimethylsulfamoyl)carbamate and methyl 2-bromoacetate. ESI-MS (m/z): 517.66, 519.71 [M+1]⁺.

### Step 2: methyl 2-(6-bromo-3-(2-((N,N-dimethylsulfamoyl)amino)ethyl)-1H-indol-1-yl)acetate

The title compound was prepared following the same general protocol as described for Example 25, using methyl 2-(6-bromo-3-(2-((tert-butoxycarbonyl)(N,N-dimethylsulfamoyl)amino)ethyl)-1H-indol-1-yl)acetate. ESI-MS (m/z): 417.71, 419.71 [M+1]⁺.

### Example 50: 2-(6-bromo-3-(2-((N,N-dimethylsulfamoyl)amino)ethyl)-1H-indol-1-yl)-N-methylacetamide (166)

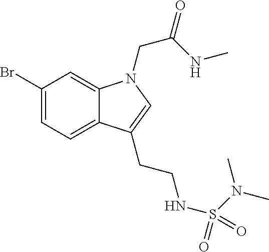 [SMILES: CNC(=O)Cn1cc(CCNS(=O)(=O)N(C)C)c2ccc(Br)cc21]

### Step 1: 2-(6-bromo-3-(2-((tert-butoxycarbonyl)(N,N-dimethylsulfamoyl)amino)ethyl)-1H-indol-1-yl)acetic acid

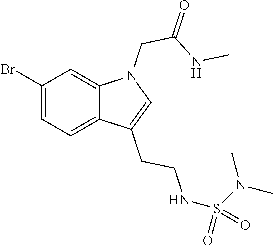 [SMILES: CNC(=O)Cn1cc(CCNS(=O)(=O)N(C)C)c2ccc(Br)cc21]

To a solution of 2-(6-bromo-3-(2-((tert-butoxycarbonyl)(N,N-dimethylsulfamoyl)amino)ethyl)-1H-indol-1-yl)acetate (0.11 g, 0.20 mmol) in THF/H₂O (2 mL, 1/1) was added NaOH (0.16 mL, 5M, 0.81 mmol). The reaction was monitored by reverse phase analytical HPLC. The solution was acidified with 1 N HCl to pH˜4 and then concentrated in vacuo to obtain the title compound with no purification. ESI-MS (m/z): 503.81, 505.82 [M+1]⁺.

### Step 2: tert-butyl (2-(6-bromo-1-(2-(methylamino)-2-oxoethyl)-1H-indol-3-yl)ethyl)(N,N-dimethylsulfamoyl)carbamate

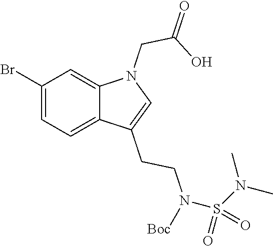 [SMILES: CN(C)S(=O)(=O)N(CCc1cn(CC(=O)O)c2cc(Br)ccc12)C(=O)OC(C)(C)C]

To a solution of 2-(6-bromo-3-(2-((tert-butoxycarbonyl)(N,N-dimethylsulfamoyl)amino)ethyl)-1H-indol-1-yl)acetic acid (0.05 g, 0.1 mmol), methyl amine HCl (0.016 g, 0.2 mmol) and HATU (0.057 g, 0.15 mmol) in DMF was added DIEA (0.09 mL, 0.5 mmol). The reaction was monitored by reverse phase analytical HPLC. When complete, the solution was diluted with EtOAc, washed with sat. aq. NaHCO₃ and brine and dried over anhydrous Na₂SO₄. The organic solution was filtered and then concentrated in vacuo to obtain the title compound with no purification. ESI-MS (m/z): 516.70, 518.75 [M+1]⁺.

### Step 3: 2-(6-bromo-3-(2-((N,N-dimethylsulfamoyl)amino)ethyl)-1H-indol-1-yl)-N-methylacetamide

The title compound was prepared following the same general protocol as described for Example 25, using tert-butyl (2-(6-bromo-1-(2-(methylamino)-2-oxoethyl)-1H-indol-3-yl)ethyl)(N,N-dimethylsulfamoyl)carbamate. ESI-MS (m/z): 416.70, 418.75 [M+1]⁺.

### Example 51: 2-(6-bromo-3-(2-((N,N-dimethylsulfamoyl)amino)ethyl)-1H-indol-1-yl)-N,N-dimethylacetamide (167)

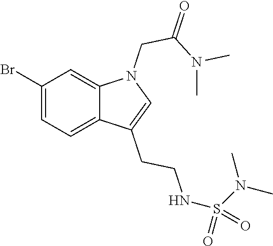 [SMILES: CN(C)C(=O)Cn1cc(CCNS(=O)(=O)N(C)C)c2ccc(Br)cc21]

### Step 1: tert-butyl (2-(6-bromo-1-(2-(dimethylamino)-2-oxoethyl)-1H-indol-3-yl)ethyl)(N,N-dimethylsulfamoyl)carbamate

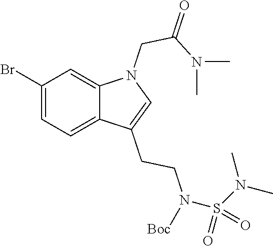 [SMILES: CN(C)C(=O)Cn1cc(CCN(C(=O)OC(C)(C)C)S(=O)(=O)N(C)C)c2ccc(Br)cc21]

The title compound was prepared following the same general protocol as described for Step 2, Example 50, using dimethyl amine HCl. ESI-MS (m/z): 530.82, 532.83 [M+1]⁺.

### Step 2: 2-(6-bromo-3-(2-((N,N-dimethylsulfamoyl)amino)ethyl)-1H-indol-1-yl)-N,N-dimethylacetamide

The title compound was prepared following the same general protocol as described for Example 25, using tert-butyl (2-(6-bromo-1-(2-(dimethylamino)-2-oxoethyl)-1H-indol-3-yl)ethyl)(N,N-dimethylsulfamoyl)carbamate. ESI-MS (m/z): 430.74, 432.72 [M+1]⁺.

### Example 52: ({2-[6-bromo-1-(2,2-dimethylpropyl)-1H-indol-3-yl]ethyl}sulfamoyl)dimethylamine (165)

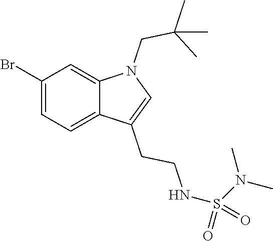 [SMILES: CN(C)S(=O)(=O)NCCc1cn(CC(C)(C)C)c2cc(Br)ccc12]

### Step 1: tert-butyl (2-(6-bromo-1-pivaloyl-1H-indol-3-yl)ethyl)(N,N-dimethylsulfamoyl)carbamate

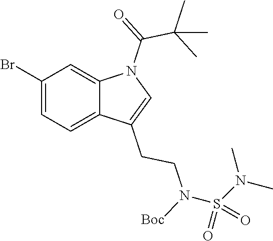 [SMILES: CN(C)S(=O)(=O)N(CCc1cn(C(=O)C(C)(C)C)c2cc(Br)ccc12)C(=O)OC(C)(C)C]

The title compound was prepared following the same general protocol as described for Example 34, using tert-butyl (2-(6-bromo-1H-indol-3-yl)ethyl)(N,N-dimethylsulfamoyl)carbamate and pivaloyl chloride. ESI-MS (m/z): 529.62, 531.66 [M+1]⁺.

### Step 2: 1-(6-bromo-3-{2-[(dimethylsulfamoyl)amino]ethyl}-1H-indol-1-yl)-2,2-dimethylpropan-1-one

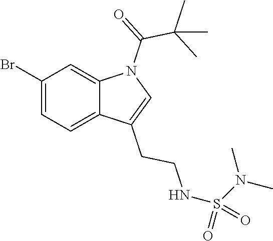 [SMILES: CN(C)S(=O)(=O)NCCc1cn(C(=O)C(C)(C)C)c2cc(Br)ccc12]

The title compound was prepared following the same general protocol as described for Example 25, using tert-butyl (2-(6-bromo-1-pivaloyl-1H-indol-3-yl)ethyl)(N,N-dimethylsulfamoyl)carbamate. ESI-MS (m/z): 429.62, 431.66 [M+1]⁺.

### Step 3: ({2-[6-bromo-1-(2,2-dimethylpropyl)-1H-indol-3-yl]ethyl}sulfamoyl)dimethylamine

The title compound was prepared following the same general protocol as described for Step 3, Example 1, using 1-(6-bromo-3-{2-[(dimethylsulfamoyl)amino]ethyl}-1H-indol-1-yl)-2,2-dimethylpropan-1-one. ESI-MS (m/z): 415.75, 417.80 [M+1]⁺.

### Example 53: 3-(2-((N,N-dimethylsulfamoyl)amino)ethyl)-N,N-dimethyl-6-(pyridin-4-yl)-1H-indole-1-carboxamide (194)

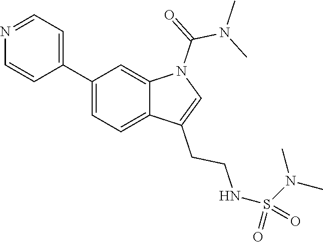 [SMILES: CN(C)C(=O)n1cc(CCNS(=O)(=O)N(C)C)c2ccc(-c3ccncc3)cc21]

### Step 1: tert-butyl (2-(6-bromo-1-(dimethylcarbamoyl)-1H-indol-3-yl)ethyl)(N,N-dimethylsulfamoyl)carbamate

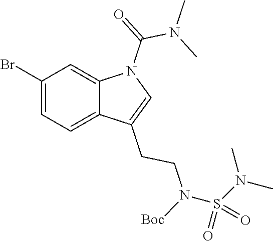 [SMILES: CN(C)C(=O)n1cc(CCN(C(=O)OC(C)(C)C)S(=O)(=O)N(C)C)c2ccc(Br)cc21]

The title compound was prepared following the same general protocol as described for Example 34, using tert-butyl (2-(6-bromo-1H-indol-3-yl)ethyl)(N,N-dimethylsulfamoyl)carbamate and dimethylcarbamic chloride. ESI-MS (m/z): 516.33, 518.47 [M+1]⁺.

### Step 2: 3-(2-((N,N-dimethylsulfamoyl)amino)ethyl)-N,N-dimethyl-6-(pyridin-4-yl)-1H-indole-1-carboxamide

The title compound was prepared following the same general protocol as described for Step 5, Example 1, using tert-butyl (2-(6-bromo-1-(dimethylcarbamoyl)-1H-indol-3-yl)ethyl)(N,N-dimethylsulfamoyl)carbamate. ESI-MS (m/z): 415.97 [M+1]⁺.

### Example 55: dimethyl({2-[1-(2-methylpropyl)-6-(trifluoromethyl)-1H-indol-3-yl]ethyl}sulfamoyl)amine (105)

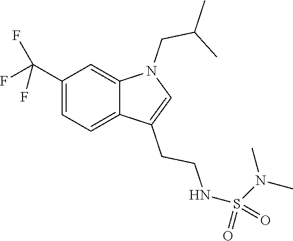 [SMILES: CC(C)Cn1cc(CCNS(=O)(=O)N(C)C)c2ccc(C(F)(F)F)cc21]

### Step 1: 1-isobutyl-6-(trifluoromethyl)-1H-indole

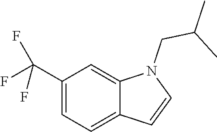 [SMILES: CC(C)Cn1ccc2ccc(C(F)(F)F)cc21]

The title compound was prepared following the same general protocol as described for Example 34, using 6-(trifluoromethyl)-1H-indole and 1-bromo-2-methylpropane. ESI-MS (m/z): 241.99 [M+1]⁺.

### Step 2: 2-(1-isobutyl-6-(trifluoromethyl)-1H-indol-3-yl)-2-oxoacetamide

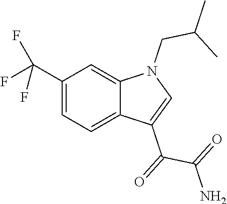 [SMILES: CC(C)Cn1cc(C(=O)C(N)=O)c2ccc(C(F)(F)F)cc21]

The title compound was prepared following the same general protocol as described for Step 2, Example 1, using 1-isobutyl-6-(trifluoromethyl)-1H-indole. ESI-MS (m/z): 312.83 [M+1]⁺.

### Step 3: 2-(1-isobutyl-6-(trifluoromethyl)-1H-indol-3-yl)ethan-1-amine

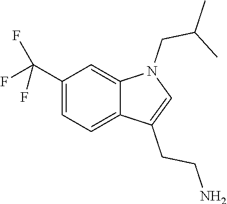 [SMILES: CC(C)Cn1cc(CCN)c2ccc(C(F)(F)F)cc21]

The title compound was prepared following the same general protocol as described for Step 3, Example 1, using 2-(1-isobutyl-6-(trifluoromethyl)-1H-indol-3-yl)-2-oxoacetamide. ESI-MS (m/z): 284.77 [M+1]⁺.

### Step 4: dimethyl({2-[1-(2-methylpropyl)-6-(trifluoromethyl)-1H-indol-3-yl]ethyl}sulfamoyl)amine

The title compound was prepared following the same general protocol as described for Step 4, Example 1, using 2-(1-isobutyl-6-(trifluoromethyl)-1H-indol-3-yl)ethan-1-amine. ESI-MS (m/z): 391.84 [M+1]⁺.

### Example 56: ({2-[6-chloro-1-(2-methylpropyl)-1H-indol-3-yl]ethyl}sulfamoyl)dimethylamine (109)

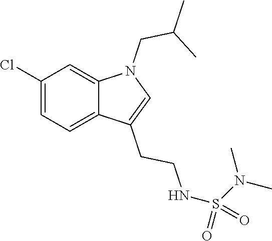 [SMILES: CC(C)Cn1cc(CCNS(=O)(=O)N(C)C)c2ccc(Cl)cc21]

### Step 1: 6-chloro-1-isobutyl-1H-indole

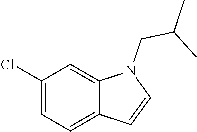

The title compound was prepared following the same general protocol as described for Example 34, using 6-chloro-1H-indole and 1-bromo-2-methylpropane. ESI-MS (m/z): 207.95 [M+1]⁺.

### Step 2: 2-(6-chloro-1-isobutyl-1H-indol-3-yl)-2-oxoacetamide

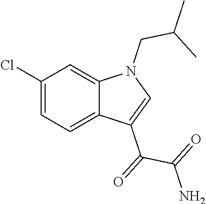 [SMILES: CC(C)Cn1cc(C(=O)C(N)=O)c2ccc(Cl)cc21]

The title compound was prepared following the same general protocol as described for Step 2, Example 1, using 6-chloro-1-isobutyl-1H-indole. ESI-MS (m/z): 278.73 [M+1]⁺.

### Step 3: 2-(6-chloro-1-isobutyl-1H-indol-3-yl)ethan-1-amine

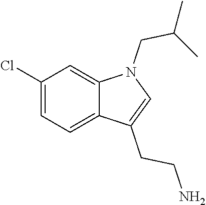 [SMILES: CC(C)Cn1cc(CCN)c2ccc(Cl)cc21]

The title compound was prepared following the same general protocol as described for Step 3, Example 1, using 2-(6-chloro-1-isobutyl-1H-indol-3-yl)-2-oxoacetamide. ESI-MS (m/z): 250.69 [M+1]⁺.

### Step 4: ({2-[6-chloro-1-(2-methylpropyl)-1H-indol-3-yl]ethyl}sulfamoyl)dimethylamine

The title compound was prepared following the same general protocol as described for Step 4, Example 1, using 2-(6-chloro-1-isobutyl-1H-indol-3-yl)ethan-1-amine.

ESI-MS (m/z): 357.76 [M+1]⁺.

### Example 57: ({2-[6-fluoro-1-(2-methylpropyl)-1H-indol-3-yl]ethyl}sulfamoyl)dimethylamine (110)

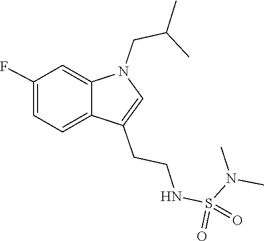 [SMILES: CC(C)Cn1cc(CCNS(=O)(=O)N(C)C)c2ccc(F)cc21]

### Step 1: 6-fluoro-1-isobutyl-1H-indole

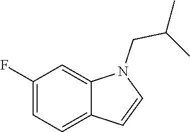

The title compound was prepared following the same general protocol as described for Example 34, using 6-fluoro-1H-indole and 1-bromo-2-methylpropane. ESI-MS (m/z): 191.88 [M+1]⁺.

### Step 2: 2-(6-fluoro-1-isobutyl-1H-indol-3-yl)-2-oxoacetamide

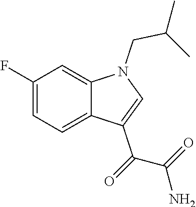 [SMILES: CC(C)Cn1cc(C(=O)C(N)=O)c2ccc(F)cc21]

The title compound was prepared following the same general protocol as described for Step 2, Example 1, using 6-fluro-1-isobutyl-1H-indole. ESI-MS (m/z): 262.72 [M+1]⁺.

### Step 3: 2-(6-fluro-1-isobutyl-1H-indol-3-yl)ethan-1-amine

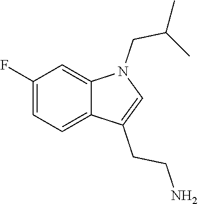 [SMILES: CC(C)Cn1cc(CCN)c2ccc(F)cc21]

The title compound was prepared following the same general protocol as described for Step 3, Example 1, using 2-(6-fluoro-1-isobutyl-1H-indol-3-yl)-2-oxoacetamide. ESI-MS (m/z): 234.74 [M+1]⁺.

### Step 4: ({2-[6-fluoro-1-(2-methylpropyl)-1H-indol-3-yl]ethyl}sulfamoyl)dimethylamine

The title compound was prepared following the same general protocol as described for Step 4, Example 1, using 2-(6-fluoro-1-isobutyl-1H-indol-3-yl)ethan-1-amine. ESI-MS (m/z): 341.77 [M+1]⁺.

### Example 58: ({2-[5-bromo-1-(2-methylpropyl)-1H-indol-3-yl]ethyl}sulfamoyl)dimethylamine (135)

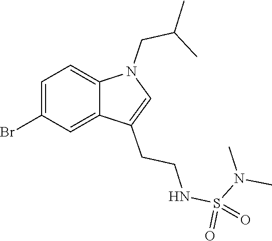 [SMILES: CC(C)Cn1cc(CCNS(=O)(=O)N(C)C)c2cc(Br)ccc21]

### Step 1: 5-bromo-1-isobutyl-1H-indole

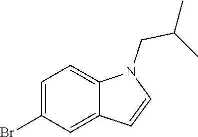

The title compound was prepared following the same general protocol as described for Example 34, using 5-bromo-1H-indole and 1-bromo-2-methylpropane. ESI-MS (m/z): 251.86, 253.86 [M+1]⁺.

### Step 2: 2-(5-bromo-1-isobutyl-1H-indol-3-yl)-2-oxoacetamide

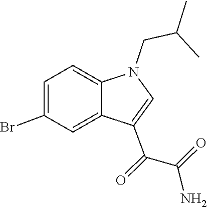 [SMILES: CC(C)Cn1cc(C(=O)C(N)=O)c2cc(Br)ccc21]

The title compound was prepared following the same general protocol as described for Step 2, Example 1, using 5-bromo-1-isobutyl-1H-indole. ESI-MS (m/z): 322.58, 324.61 [M+1]⁺.

### Step 3: 2-(5-bromo-1-isobutyl-1H-indol-3-yl)ethan-1-amine

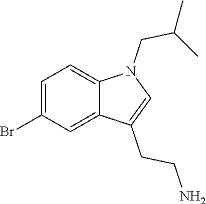 [SMILES: CC(C)Cn1cc(CCN)c2cc(Br)ccc21]

The title compound was prepared following the same general protocol as described for Step 3, Example 1, using 2-(5-bromo-1-isobutyl-1H-indol-3-yl)-2-oxoacetamide. ESI-MS (m/z): 294.66, 296.69 [M+1]⁺.

### Step 4: ({2-[5-bromo-1-(2-methylpropyl)-1H-indol-3-yl]ethyl}sulfamoyl)dimethylamine

The title compound was prepared following the same general protocol as described for Step 4, Example 1, using 2-(5-bromo-1-isobutyl-1H-indol-3-yl)ethan-1-amine. ESI-MS (m/z): 401.70, 403.71 [M+1]⁺.

### Example 59: ({2-[4-bromo-1-(2-methylpropyl)-1H-indol-3-yl]ethyl}sulfamoyl)dimethylamine (136)

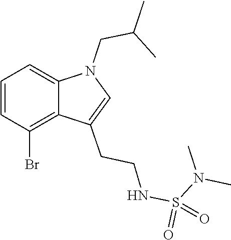 [SMILES: CC(C)Cn1cc(CCNS(=O)(=O)N(C)C)c2c(Br)cccc21]

### Step 1: 4-bromo-1-isobutyl-1H-indole

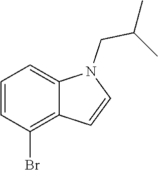

The title compound was prepared following the same general protocol as described for Example 34, using 4-bromo-1H-indole and 1-bromo-2-methylpropane. ESI-MS (m/z): 251.86, 253.86 [M+1]⁺.

### Step 2: 2-(4-bromo-1-isobutyl-1H-indol-3-yl)-2-oxoacetamide

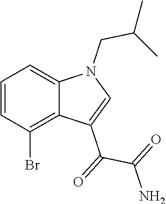 [SMILES: CC(C)Cn1cc(C(=O)C(N)=O)c2c(Br)cccc21]

The title compound was prepared following the same general protocol as described for Step 2, Example 1, using 4-bromo-1-isobutyl-1H-indole. ESI-MS (m/z): 322.58, 324.61 [M+1]⁺.

### Step 3: 2-(4-bromo-1-isobutyl-1H-indol-3-yl)ethan-1-amine

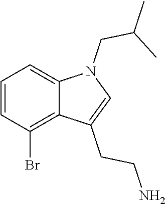 [SMILES: CC(C)Cn1cc(CCN)c2c(Br)cccc21]

The title compound was prepared following the same general protocol as described for Step 3, Example 1, using 2-(4-bromo-1-isobutyl-1H-indol-3-yl)-2-oxoacetamide. ESI-MS (m/z): 294.77, 296.72 [M+1]⁺.

### Step 4: ({2-[4-bromo-1-(2-methylpropyl)-1H-indol-3-yl]ethyl}sulfamoyl)dimethylamine

The title compound was prepared following the same general protocol as described for Step 4, Example 1, using 2-4-bromo-1-isobutyl-1H-indol-3-yl)ethan-1-amine. ESI-MS (m/z): 401.66, 403.73 [M+1]⁺.

### Example 60: ({2-[7-bromo-1-(2-methylpropyl)-1H-indol-3-yl]ethyl}sulfamoyl)dimethylamine (137)

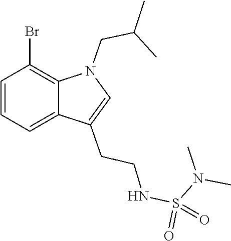 [SMILES: CC(C)Cn1cc(CCNS(=O)(=O)N(C)C)c2cccc(Br)c21]

### Step 1: 7-bromo-1-isobutyl-1H-indole

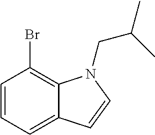

The title compound was prepared following the same general protocol as described for Example 34, using 7-bromo-1H-indole and 1-bromo-2-methylpropane. ESI-MS (m/z): 251.86, 253.86 [M+1]⁺.

### Step 2: 2-(7-bromo-1-isobutyl-1H-indol-3-yl)-2-oxoacetamide

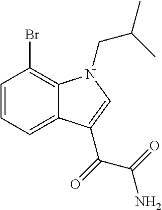 [SMILES: CC(C)Cn1cc(C(=O)C(N)=O)c2cccc(Br)c21]

The title compound was prepared following the same general protocol as described for Step 2, Example 1, using 7-bromo-1-isobutyl-1H-indole. ESI-MS (m/z): 322.58, 324.61 [M+1]⁺.

### Step 3: 2-(7-bromo-1-isobutyl-1H-indol-3-yl)ethan-1-amine

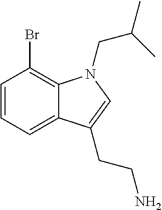 [SMILES: CC(C)Cn1cc(CCN)c2cccc(Br)c21]

The title compound was prepared following the same general protocol as described for Step 3, Example 1, using 2-(7-bromo-1-isobutyl-1H-indol-3-yl)-2-oxoacetamide. ESI-MS (m/z): 294.69, 296.70 [M+1]⁺.

### Step 4: ({2-[7-bromo-1-(2-methylpropyl)-1H-indol-3-yl]ethyl}sulfamoyl)dimethylamine

The title compound was prepared following the same general protocol as described for Step 4, Example 1, using 2-7-bromo-1-isobutyl-1H-indol-3-yl)ethan-1-amine. ESI-MS (m/z): 401.66, 403.73 [M+1]⁺.

### Example 61: dimethyl({2-[7-methyl-1-(2-methylpropyl)-1H-indol-3-yl]ethyl}sulfamoyl)amine (169)

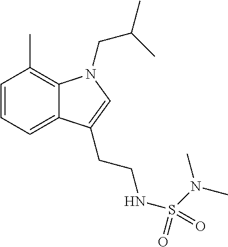 [SMILES: Cc1cccc2c(CCNS(=O)(=O)N(C)C)cn(CC(C)C)c12]

The title compound was prepared following the same general protocol as described for Step 5, Example 1, using 2,4,6-trimethyl-1,3,5,2,4,6-trioxatriborinane and ({2-[7-bromo-1-(2-methylpropyl)-1H-indol-3-yl]ethyl}sulfamoyl)dimethylamine. ESI-MS (m/z): 337.79 [M+1]⁺.

### Example 62: dimethyl[methyl({2-[1-(2-methylpropyl)-6-(trifluoromethyl)-1H-indol-3-yl]ethyl})sulfamoyl]amine (121)

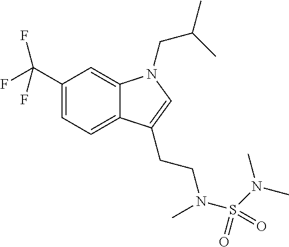 [SMILES: CC(C)Cn1cc(CCN(C)S(=O)(=O)N(C)C)c2ccc(C(F)(F)F)cc21]

The title compound was prepared following the same general protocol as described for Example 34, using dimethyl({2-[1-(2-methylpropyl)-6-(trifluoromethyl)-1H-indol-3-yl]ethyl}sulfamoyl)amine. ESI-MS (m/z): 405.76 [M+1]⁺.

### Example 63: ({2-[6-tert-butyl-1-(2-methylpropyl)-1H-indol-3-yl]ethyl}sulfamoyl)dimethylamine (106)

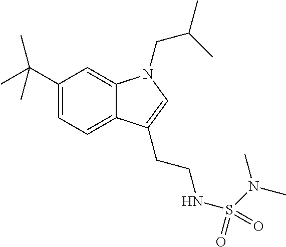 [SMILES: CC(C)Cn1cc(CCNS(=O)(=O)N(C)C)c2ccc(C(C)(C)C)cc21]

### Step 1: (E)-2-(4-(tert-butyl)-2-nitrophenyl)-N,N-dimethylethen-1-amine

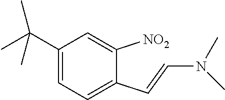 [SMILES: CN(C)/C=C/c1ccc(C(C)(C)C)cc1[N+](=O)[O-]]

A solution of 4-(tert-butyl)-1-methyl-2-nitrobenzene (2.26 g, 11.715 mmol) and DMF-DMA (2.0 mL, 15.23 mmol) in DMF (12 mL) was heated in a 150° C. oil bath for 2 days. The solvent was removed in vacuo and the crude was dissolved with EtOAc, washed with brine. The organic layer was dried (MgSO₄), filtered, and concentrated in vacuo. The crude residue was used without further purification. ESI-MS (m/z): 249.02 [M+1]⁺.

### Step 2: 6-(tert-butyl)-1H-indole

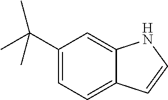

To a solution of the crude residue from previous step in benzene (20 mL) was added Pd/C (0.1 g). The solution was stirred under a H₂ balloon overnight. The solution was filtered through a celite Pad and the filtrate was concentrated in vacuo. The crude was purified by chromatography on silica gel (EtOAc/hex) to afford the title compound. ESI-MS (m/z): 173.83 [M+1]⁺. ¹HNMR (CDCl₃, 400 MHz) δ 7.56 (d, J=8.4 Hz, 1H), 7.29 (s, 1H), 7.19 (dd, J=8.4 Hz, 1.6 Hz, 1H), 7.02 (d, J=2.8 Hz, 1H), 6.41 (t, J=2.8 Hz, 1H), 3.90 (d, J=7.6 Hz, 2H), 2.20 (m, 1H), 0.95 (d, J+6.8 Hz, 6H).

### Step 3: 6-(tert-butyl)-1-isobutyl-1H-indole

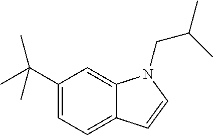 [SMILES: CC(C)Cn1ccc2ccc(C(C)(C)C)cc21]

The title compound was prepared following the same general protocol as described for Example 34, using 6-tert-butyl-1H-indole and 1-bromo-2-methylpropane. ESI-MS (m/z): 230.12 [M+1]⁺.

### Step 4: 2-(6-(tert-butyl)-1-isobutyl-1H-indol-3-yl)-2-oxoacetamide

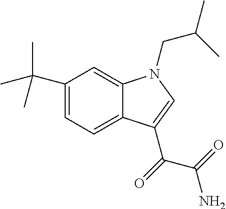 [SMILES: CC(C)Cn1cc(C(=O)C(N)=O)c2ccc(C(C)(C)C)cc21]

The title compound was prepared following the same general protocol as described for Step 2, Example 1, using 6-tert-butyl-1-isobutyl-1H-indole. ESI-MS (m/z): 301.02 [M+1]⁺.

### Step 5: 2-(6-tert-butyl-1-isobutyl-1H-indol-3-yl)ethan-1-amine

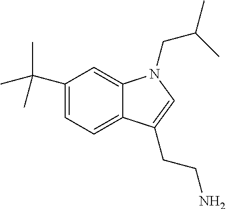 [SMILES: CC(C)Cn1cc(CCN)c2ccc(C(C)(C)C)cc21]

The title compound was prepared following the same general protocol as described for Step 3, Example 1, using 2-(6-tert-butyl-1-isobutyl-1H-indol-3-yl)-2-oxoacetamide. ESI-MS (m/z): 272.85 [M+1]⁺.

### Step 6: ({2-[6-tert-butyl-1-(2-methylpropyl)-1H-indol-3-yl]ethyl}sulfamoyl)dimethylamine

The title compound was prepared following the same general protocol as described for Step 4, Example 1, using 2-(6-tert-butyl-1-isobutyl-1H-indol-3-yl)ethan-1-amine. ESI-MS (m/z): 379.92 [M+1]⁺.

### Example 64: ({2-[5,6-dimethyl-1-(2-methylpropyl)-1H-indol-3-yl]ethyl}sulfamoyl)dimethylamine (120)

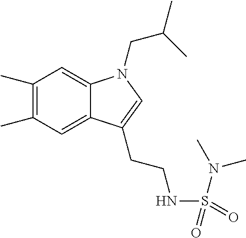

### Step 1: (E)-1-(4,5-dimethyl-2-nitrostyryl)pyrrolidine

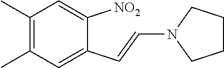 [SMILES: Cc1cc(/C=C/N2CCCC2)c([N+](=O)[O-])cc1C]

The title compound was prepared following the same general protocol as described for Step 1, Example 63, using 1,2,4-trimethyl-5-nitrobenzene and pyrrolidine. ESI-MS (m/z): 247.12 [M+1]⁺.

### Step 2: 5,6-dimethyl-1H-indole

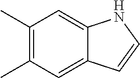

The title compound was prepared following the same general protocol as described for Step 2, Example 63, using (E)-1-(4,5-dimethyl-2-nitrostyryl)pyrrolidine. ESI-MS (m/z): 145.97 [M+1]⁺.

### Step 3: 1-isobutyl-5,6-dimethyl-1H-indole

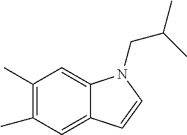

The title compound was prepared following the same general protocol as described for Example 34, using 5,6-dimethyl-1H-indole and 1-bromo-2-methylpropane. ESI-MS (m/z): 201.92 [M+1]⁺.

### Step 4: 2-(1-isobutyl-5,6-dimethyl-1H-indol-3-yl)-2-oxoacetamide

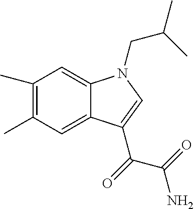 [SMILES: Cc1cc2c(C(=O)C(N)=O)cn(CC(C)C)c2cc1C]

The title compound was prepared following the same general protocol as described for Step 2, Example 1, using 1-isobutyl-5,6-dimethyl-1H-indole. ESI-MS (m/z): 272.69 [M+1]⁺.

### Step 5: 2-(1-isobutyl-5,6-dimethyl-1H-indol-3-yl)ethan-1-amine

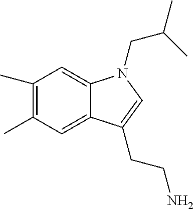 [SMILES: Cc1cc2c(CCN)cn(CC(C)C)c2cc1C]

The title compound was prepared following the same general protocol as described for Step 3, Example 1, using 2-(1-isobutyl-5,6-dimethyl-1H-indol-3-yl)-2-oxoacetamide. ESI-MS (m/z): 244.67 [M+1]⁺.

### Step 6: ({2-[5,6-dimethyl-1-(2-methylpropyl)-1H-indol-3-yl]ethyl}sulfamoyl)dimethylamine

The title compound was prepared following the same general protocol as described for Step 4, Example 1, using 2-(1-isobutyl-5,6-dimethyl-1H-indol-3-yl)ethan-1-amine. ESI-MS (m/z): 351.87 [M+1]⁺.

### Example 65: 1-(3-{2-[(dimethylsulfamoyl)amino]ethyl}-1-(2-methylpropyl)-1H-indol-6-yl)cyclobutan-1-ol (107)

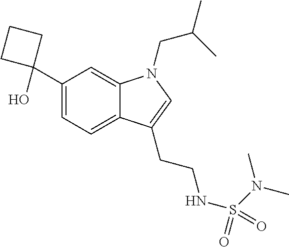 [SMILES: CC(C)Cn1cc(CCNS(=O)(=O)N(C)C)c2ccc(C3(O)CCC3)cc21]

### Step 1: 1-(1-isobutyl-1H-indol-6-yl)cyclobutan-1-ol

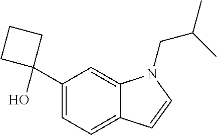 [SMILES: CC(C)Cn1ccc2ccc(C3(O)CCC3)cc21]

To a solution of 6-bromo-1-isobutyl-1H-indole (2.403 g, 9.53 mmol) in THF (20 mL) at −78° C. under argon was added dropwise nBuLi (4.0 mL, 2.5 M, 10.0 mmol). After 20 min at −78° C., a solution of cyclobutanone (0.71 mL, 9.53 mmol) in THF (5 mL) was added dropwise. The reaction was stirred 30 min at −78° C. and then warmed to RT. Water was added, and the solution was extracted with EtOAc. The organic layers were combined, dried (MgSO₄) and concentrated in vacuo to obtain the crude which was purified by chromatography on silica gel (EtOAc/hex) to afford the title compound. ESI-MS (m/z): 244.01 [M+1]⁺. ¹HNMR (CDCl₃, 400 MHz) δ 7.63 (d, J=8.4 Hz, 1H), 7.45 (s, 1H), 7.24 (d, J=1.6 Hz, 1H), 7.09 (d, J=3.2 Hz, 1H), 6.47 (d, J=3.2 Hz, 1H), 3.93 (d, J=7.6 Hz, 2H), 2.68 (m, 2H), 2.47 (m, 2H), 2.25 (m, 1H), 2.06 (m, 2H), 1.72 (m, 1H), 0.96 (d, J=7.6 Hz, 6H).

### Step 2: 2-(6-(1-hydroxycyclobutyl)-1-isobutyl-1H-indol-3-yl)-2-oxoacetamide

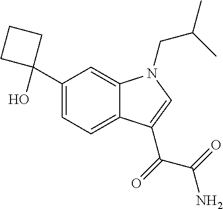 [SMILES: CC(C)Cn1cc(C(=O)C(N)=O)c2ccc(C3(O)CCC3)cc21]

The title compound was prepared following the same general protocol as described for Step 2, Example 1, using 1-(1-isobutyl-1H-indol-6-yl)cyclobutan-1-ol. ESI-MS (m/z): 314.89 [M+1]⁺.

### Step 3: 1-(3-(2-aminoethyl)-1-isobutyl-1H-indol-6-yl)cyclobutan-1-ol

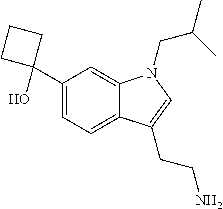 [SMILES: CC(C)Cn1cc(CCN)c2ccc(C3(O)CCC3)cc21]

The title compound was prepared following the same general protocol as described for Step 3, Example 1, using 2-(6-(1-hydroxycyclobutyl)-1-isobutyl-1H-indol-3-yl)-2-oxoacetamide. ESI-MS (m/z): 286.88 [M+1]⁺.

### Step 4: 1-(3-{2-[(dimethylsulfamoyl)amino]ethyl}-1-(2-methylpropyl)-1H-indol-6-yl)cyclobutan-1-ol

The title compound was prepared following the same general protocol as described for Step 4, Example 1, using 1-(3-(2-aminoethyl)-1-isobutyl-1H-indol-6-yl)cyclobutan-1-ol. ESI-MS (m/z): 393.89 [M+1]⁺.

### Example 66: ({2-[6-cyclobutyl-1-(2-methylpropyl)-1H-indol-3-yl]ethyl}sulfamoyl)dimethylamine (108)

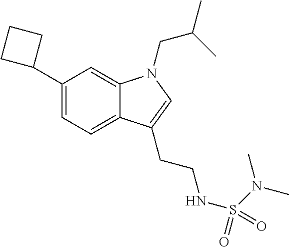 [SMILES: CC(C)Cn1cc(CCNS(=O)(=O)N(C)C)c2ccc(C3CCC3)cc21]

### Step 1: 6-cyclobutyl-1-isobutyl-1H-indole

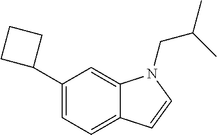 [SMILES: CC(C)Cn1ccc2ccc(C3CCC3)cc21]

To a solution of 1-(1-isobutyl-1H-indol-6-yl)cyclobutan-1-ol (0.99 g, 4.05 mmol) in DCM (12 mL) at −78° C. under argon was added triethylsilane (0.78 mL, 4.858 mmol), followed by slow addition of BF₃·OEt₂ (0.8 mL, 6.5 mmol). The reaction was gradually warmed to RT and stirred overnight. The reaction solution was diluted with DCM, washed with water, brine, dried (MgSO₄) and concentrated in vacuo. The crude residue was purified by chromatography on silica gel (EtOAc/hex) to afford the title compound. ESI-MS (m/z): 228.01 [M+1]⁺.

### Step 2: 2-(6-cyclobutyl-1-isobutyl-1H-indol-3-yl)-2-oxoacetamide

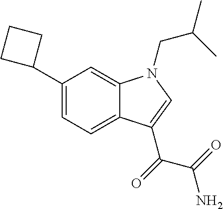 [SMILES: CC(C)Cn1cc(C(=O)C(N)=O)c2ccc(C3CCC3)cc21]

The title compound was prepared following the same general protocol as described for Step 2, Example 1, using 6-cyclobutyl-1-isobutyl-1H-indole. ESI-MS (m/z): 298.82 [M+1]⁺.

### Step 3: 2-(6-cyclobutyl-1-isobutyl-1H-indol-3-yl)ethan-1-amine

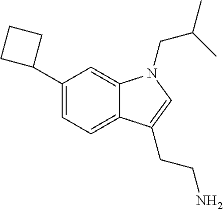 [SMILES: CC(C)Cn1cc(CCN)c2ccc(C3CCC3)cc21]

The title compound was prepared following the same general protocol as described for Step 3, Example 1, using 2-(6-cyclobutyl-1-isobutyl-1H-indol-3-yl)-2-oxoacetamide. ESI-MS (m/z): 270.77 [M+1]⁺.

### Step 4: ({2-[6-cyclobutyl-1-(2-methylpropyl)-1H-indol-3-yl]ethyl}sulfamoyl)dimethylamine

The title compound was prepared following the same general protocol as described for Step 4, Example 1, using 2-(6-cyclobutyl-1-isobutyl-1H-indol-3-yl)ethan-1-amine. ESI-MS (m/z): 377.88 [M+1]⁺.

### Example 67: ({2-[6-bromo-5-fluoro-1-(2-methylpropyl)-1H-indol-3-yl]ethyl}sulfamoyl)dimethylamine (168)

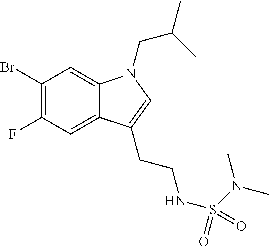 [SMILES: CC(C)Cn1cc(CCNS(=O)(=O)N(C)C)c2cc(F)c(Br)cc21]

### Step 1: (E)-2-(4-bromo-5-fluoro-2-nitrophenyl)-N,N-dimethylethen-1-amine

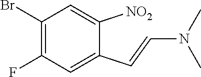 [SMILES: CN(C)/C=C/c1cc(F)c(Br)cc1[N+](=O)[O-]]

The title compound was prepared following the same general protocol as described for Step 1, Example 63, using 1-bromo-2-fluoro-4-methyl-5-nitrobenzene.

### Step 2: 6-bromo-5-fluoro-1H-indole

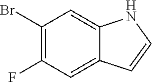 [SMILES: Fc1cc2cc[nH]c2cc1Br]

To a solution of (E)-2-(4-bromo-5-fluoro-2-nitrophenyl)-N,N-dimethylethen-1-amine (7.23 g, 25.0 mmol) in EtOH (120 mL) and AcOH (120 mL) was added iron powder (14 g, 250 mmol). The solution was heated at 90° C. overnight. The solution was cooled to RT and filtered through celite. The celite pad was washed with MeOH. The filtrates were concentrated in vacuo and then re-dissolved in EtOAc, washed with water, sat'd NaHCO₃, brine, dried (MgSO₄) and filtered. The organic layer was concentrated to obtain the crude which was purified by chromatography on silica gel (EtOAc/hex) to afford the title compound. ESI-MS (m/z): 213.88, 215.85 [M+1]⁺.

### Step 3: 6-bromo-5-fluoro-1-isobutyl-1H-indole

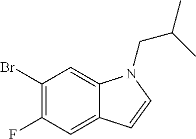

The title compound was prepared following the same general protocol as described for Example 34, using 6-bromo-5-fluoro-1H-indole. ESI-MS (m/z): 269.75, 271.78 [M+1]⁺.

### Step 4: 2-(6-bromo-5-fluoro-1-isobutyl-1H-indol-3-yl)-2-oxoacetamide

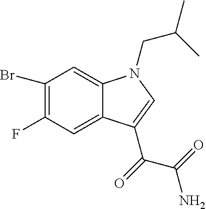 [SMILES: CC(C)Cn1cc(C(=O)C(N)=O)c2cc(F)c(Br)cc21]

The title compound was prepared following the same general protocol as described for Step 2, Example 1, using 6-bromo-5-fluoro-1-isobutyl-1H-indole. ESI-MS (m/z): 340.59, 342.63 [M+1]⁺.

### Step 5: 2-(6-bromo-5-fluoro-1-isobutyl-1H-indol-3-yl)ethan-1-amine

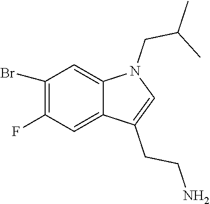 [SMILES: CC(C)Cn1cc(CCN)c2cc(F)c(Br)cc21]

The title compound was prepared following the same general protocol as described for Step 3, Example 1, using 2-(6-bromo-5-fluoro-1-isobutyl-1H-indol-3-yl)-2-oxoacetamide. ESI-MS (m/z): 312.72, 314.70 [M+1]⁺.

### Step 6: ({2-[6-bromo-5-fluoro-1-(2-methylpropyl)-1H-indol-3-yl]ethyl}sulfamoyl)dimethylamine

The title compound was prepared following the same general protocol as described for Step 4, Example 1, using 2-(6-bromo-5-fluoro-1-isobutyl-1H-indol-3-yl)ethan-1-amine. ESI-MS (m/z): 419.68, 421.68 [M+1]⁺.

### Example 68: ({2-[5-fluoro-6-methyl-1-(2-methylpropyl)-1H-indol-3-yl]ethyl}sulfamoyl)dimethylamine (170)

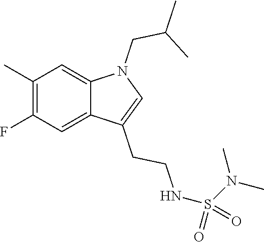 [SMILES: Cc1cc2c(cc1F)c(CCNS(=O)(=O)N(C)C)cn2CC(C)C]

The title compound was prepared following the same general protocol as described for Step 5, Example 1, using 2,4,6-trimethyl-1,3,5,2,4,6-trioxatriborinane and ({2-[6-bromo-5-fluoro-1-(2-methylpropyl)-1H-indol-3-yl]ethyl}sulfamoyl)dimethylamine. ESI-MS (m/z): 355.79 [M+1]⁺.

### Example 69: ({2-[5-fluoro-1-(2-methylpropyl)-6-(pyridin-4-yl)-1H-indol-3-yl]ethyl}sulfamoyl)dimethylamine (176)

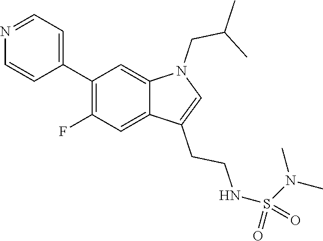 [SMILES: CC(C)Cn1cc(CCNS(=O)(=O)N(C)C)c2cc(F)c(-c3ccncc3)cc21]

The title compound was prepared following the same general protocol as described for Step 5, Example 1, using pyridin-4-ylboronic acid and ({2-[6-bromo-5-fluoro-1-(2-methylpropyl)-1H-indol-3-yl]ethyl}sulfamoyl)dimethylamine. ESI-MS (m/z): 418.97 [M+1]⁺.

### Example 71: 3-(1-isobutyl-6-phenyl-1H-indol-3-yl)-N,N-dimethylpiperidine-1-sulfonamide (61)

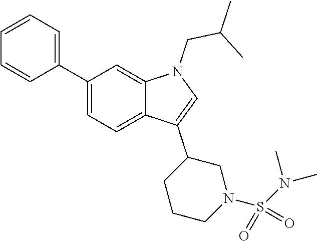 [SMILES: CC(C)Cn1cc(C2CCCN(S(=O)(=O)N(C)C)C2)c2ccc(-c3ccccc3)cc21]

### Step 1: 1-isobutyl-6-phenyl-3-(piperidin-3-yl)-1H-indole

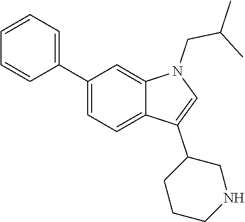 [SMILES: CC(C)Cn1cc(C2CCCNC2)c2ccc(-c3ccccc3)cc21]

The title compound was prepared following the same general protocol as described for Example 25, using tert-butyl 3-(1-isobutyl-6-phenyl-1H-indol-3-yl)piperidine-1-carboxylate. ESI-MS (m/z): 333.00 [M+1]⁺.

### Step 2: 3-(1-isobutyl-6-phenyl-1H-indol-3-yl)-N,N-dimethylpiperidine-1-sulfonamide

The title compound was prepared following the same general protocol as described for Step 4, Example 1, using 1-isobutyl-6-phenyl-3-(piperidin-3-yl)-1H-indole. ESI-MS (m/z): 439.94 [M+1]⁺.

### Example 72: 1-isobutyl-3-(1-(isopropylsulfonyl)piperidin-3-yl)-6-phenyl-1H-indole (62)

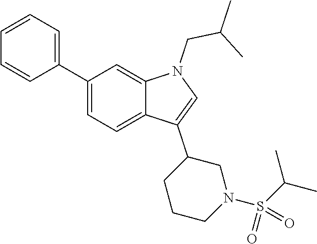 [SMILES: CC(C)Cn1cc(C2CCCN(S(=O)(=O)C(C)C)C2)c2ccc(-c3ccccc3)cc21]

The title compound was prepared following the same general protocol as described for Step 4, Example 1, using 1-isobutyl-6-phenyl-3-(piperidin-3-yl)-1H-indole and propane-2-sulfonyl chloride. ESI-MS (m/z): 439.08 [M+1]⁺.

### Example 73: 1-isobutyl-3-(1-(isopropylsulfonyl)-1H-pyrazol-4-yl)-6-phenyl-1H-indole (63)

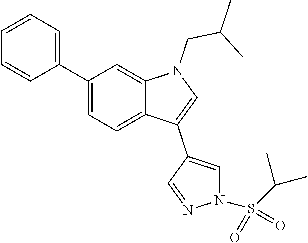 [SMILES: CC(C)Cn1cc(-c2cnn(S(=O)(=O)C(C)C)c2)c2ccc(-c3ccccc3)cc21]

### Step 1: 1-isobutyl-6-phenyl-1H-indole

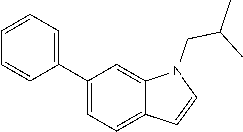 [SMILES: CC(C)Cn1ccc2ccc(-c3ccccc3)cc21]

The title compound was prepared following the same general protocol as described for Step 5, Example 1, using phenylboronic acid and 6-bromo-1-isobutyl-1H-indole. ESI-MS (m/z): 249.99 [M+1]⁺.

### Step 2: 3-bromo-1-isobutyl-6-phenyl-1H-indole

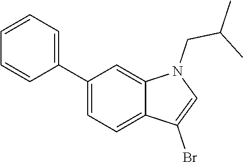 [SMILES: CC(C)Cn1cc(Br)c2ccc(-c3ccccc3)cc21]

NBS (2.12 g, 12.04 mmol) in THF (30 mL) was added dropwise to the solution of 1-isobutyl-6-phenyl-1H-indole (2.83 g, 11.36 mmol) in THF (100 mL). The reaction was stirred at RT overnight. The solution was diluted with EtOAc, washed with 5% Na₂S₂O₃, brine, dried (MgSO₄) and filtered. The filtrate was concentrated in vacuo to obtain the crude which was purified by chromatography on silica gel (EtOAc/hex) to afford the title compound. ESI-MS (m/z): 327.99, 329.99 [M+1]⁺.

### Step 3: 1-isobutyl-6-phenyl-3-(1H-pyrazol-4-yl)-1H-indole

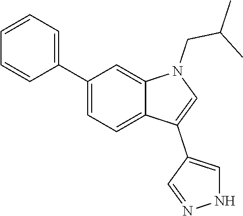 [SMILES: CC(C)Cn1cc(-c2cn[nH]c2)c2ccc(-c3ccccc3)cc21]

The title compound was prepared following the same general protocol as described for Step 5, Example 1, using tert-butyl 4-(4,4,5,5-tetramethyl-1,3,2-dioxaborolan-2-yl)-1H-pyrazole-1-carboxylate and 3-bromo-1-isobutyl-6-phenyl-1H-indole. ESI-MS (m/z): 316.10 [M+1].

### Step 4: 1-isobutyl-3-(1-(isopropylsulfonyl)-1H-pyrazol-4-yl)-6-phenyl-1H-indole

To a solution of 1-isobutyl-6-phenyl-3-(1H-pyrazol-4-yl)-1H-indole (0.008 g, 0.025 mmol) in THF (0.2 mL) was added NaH (0.002 g, 60% in suspension, 0.05 mmol). The solution was stirred for 30 min at RT and then propane-2-sulfonyl chloride (0.007 g, 0.05 mmol) was added dropwise. The reaction was quenched with the addition of MeOH, concentrated in vacuo, and then purified by prep-HPLC to obtain the title compound. ESI-MS (m/z): 421.69 [M+1]⁺.

### Example 77: N-(3-(1-isobutyl-6-phenyl-1H-indol-3-yl)phenyl)propane-2-sulfonamide (83)

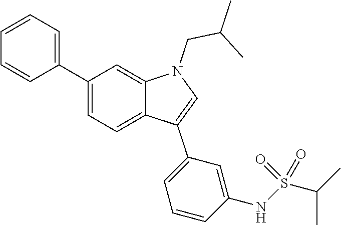 [SMILES: CC(C)Cn1cc(-c2cccc(NS(=O)(=O)C(C)C)c2)c2ccc(-c3ccccc3)cc21]

### Step 1: 3-(1-isobutyl-6-phenyl-1H-indol-3-yl)aniline

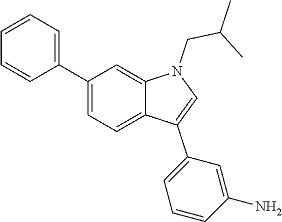 [SMILES: CC(C)Cn1cc(-c2cccc(N)c2)c2ccc(-c3ccccc3)cc21]

The title compound was prepared following the same general protocol as described for Step 5, Example 1, (3-aminophenyl)boronic acid and 3-bromo-1-isobutyl-6-phenyl-1H-indole. ESI-MS (m/z): 341.05 [M+1]⁺.

### Step 2: N-(3-(1-isobutyl-6-phenyl-1H-indol-3-yl)phenyl)propane-2-sulfonamide

Propane-2-sulfonyl chloride (0.015 g, 0.105 mmol) was added to the solution of 3-(1-isobutyl-6-phenyl-1H-indol-3-yl)aniline (0.012 g, 0.035 mmol) in pyridine (0.5 mL). The solution was stirred overnight. The solution was purified by prep-HPLC to obtain the title compound. ESI-MS (m/z): 447.07 [M+1]⁺.

### Example 78: N-(4-(1-isobutyl-6-phenyl-1H-indol-3-yl)phenyl)propane-2-sulfonamide (84)

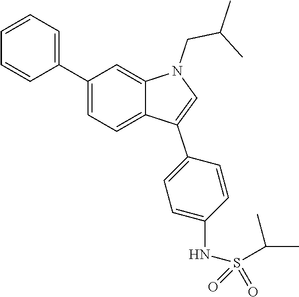 [SMILES: CC(C)Cn1cc(-c2ccc(NS(=O)(=O)C(C)C)cc2)c2ccc(-c3ccccc3)cc21]

### Step 1: 4-(1-isobutyl-6-phenyl-1H-indol-3-yl)aniline

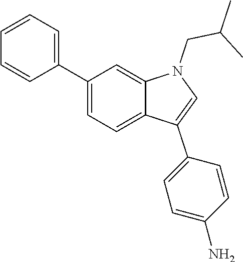 [SMILES: CC(C)Cn1cc(-c2ccc(N)cc2)c2ccc(-c3ccccc3)cc21]

The title compound was prepared following the same general protocol as described for Step 5, Example 1, using (3-aminophenyl)boronic acid and 3-bromo-1-isobutyl-6-phenyl-1H-indole. ESI-MS (m/z): 341.06 [M+1]⁺.

### Step 2: N-(3-(1-isobutyl-6-phenyl-1H-indol-3-yl)phenyl)propane-2-sulfonamide

The title compound was prepared following the same general protocol as described for Step 2, Example 77, using 4-(1-isobutyl-6-phenyl-1H-indol-3-yl)aniline. ESI-MS (m/z): 447.14 [M+1]⁺.

### Example 79: N-(3-(1-isobutyl-6-phenyl-1H-indol-3-yl)benzyl)propane-2-sulfonamide (86)

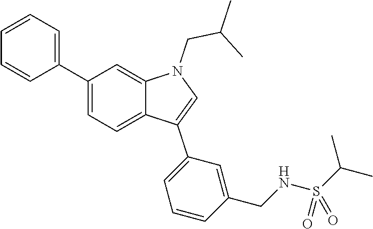 [SMILES: CC(C)Cn1cc(-c2cccc(CNS(=O)(=O)C(C)C)c2)c2ccc(-c3ccccc3)cc21]

### Step 1: tert-butyl (3-(1-isobutyl-6-phenyl-1H-indol-3-yl)benzyl)carbamate

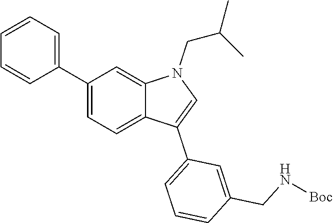 [SMILES: CC(C)Cn1cc(-c2cccc(CNC(=O)OC(C)(C)C)c2)c2ccc(-c3ccccc3)cc21]

The title compound was prepared following the same general protocol as described for Step 5, Example 1, using tert-butyl (3-(4,4,5,5-tetramethyl-1,3,2-dioxaborolan-2-yl)benzyl)carbamate and 3-bromo-1-isobutyl-6-phenyl-1H-indole. ESI-MS (m/z): 455.28 [M+1]⁺.

### Step 2: (3-(1-isobutyl-6-phenyl-1H-indol-3-yl)phenyl)methanamine

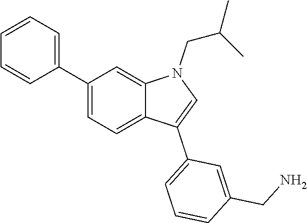 [SMILES: CC(C)Cn1cc(-c2cccc(CN)c2)c2ccc(-c3ccccc3)cc21]

The title compound was prepared following the same general protocol as described for Example 25, using tert-butyl (3-(1-isobutyl-6-phenyl-1H-indol-3-yl)benzyl)carbamate. ESI-MS (m/z): 354.81 [M+1]⁺.

### Step 3: N-(3-(1-isobutyl-6-phenyl-1H-indol-3-yl)benzyl)propane-2-sulfonamide

The title compound was prepared following the same general protocol as described for Step 4, Example 1, using (3-(1-isobutyl-6-phenyl-1H-indol-3-yl)phenyl)methanamine and propane-2-sulfonyl chloride. ESI-MS (m/z): 421.78 [M+1]⁺.

### Example 80: N-(4-(1-isobutyl-6-phenyl-1H-indol-3-yl)benzyl)propane-2-sulfonamide (87)

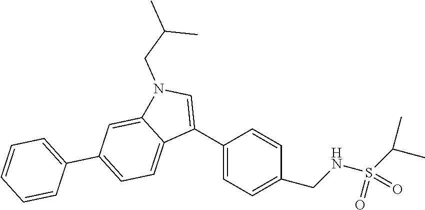 [SMILES: CC(C)Cn1cc(-c2ccc(CNS(=O)(=O)C(C)C)cc2)c2ccc(-c3ccccc3)cc21]

### Step 1: tert-butyl (4-(1-isobutyl-6-phenyl-1H-indol-3-yl)benzyl)carbamate

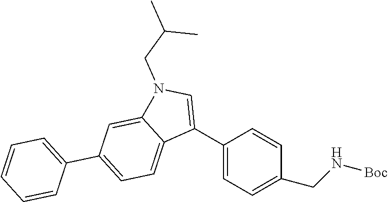 [SMILES: CC(C)Cn1cc(-c2ccc(CNC(=O)OC(C)(C)C)cc2)c2ccc(-c3ccccc3)cc21]

The title compound was prepared following the same general protocol as described for Step 5, Example 1, using (4-(((tert-butoxycarbonyl)amino)methyl)phenyl)boronic acid and 3-bromo-1-isobutyl-6-phenyl-1H-indole. ESI-MS (m/z): 455.04 [M+1]⁺.

### Step 2: (4-(1-isobutyl-6-phenyl-1H-indol-3-yl)phenyl)methanamine

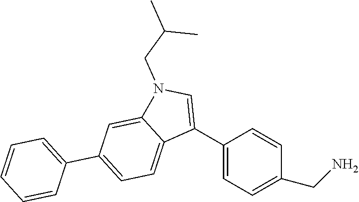 [SMILES: CC(C)Cn1cc(-c2ccc(CN)cc2)c2ccc(-c3ccccc3)cc21]

The title compound was prepared following the same general protocol as described for Example 25, using tert-butyl (4-(1-isobutyl-6-phenyl-1H-indol-3-yl)benzyl)carbamate. ESI-MS (m/z): 354.80 [M+1]⁺.

### Step 3: N-(4-(1-isobutyl-6-phenyl-1H-indol-3-yl)benzyl)propane-2-sulfonamide

The title compound was prepared following the same general protocol as described for Step 4, Example 1, using (4-(1-isobutyl-6-phenyl-1H-indol-3-yl)phenyl)methanamine and propane-2-sulfonyl chloride. ESI-MS (m/z): 461.13 [M+1]⁺.

### Example 81: N-(2-(1-isobutyl-6-phenyl-1H-indol-3-yl)phenyl)propane-2-sulfonamide (124)

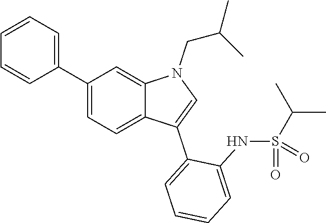 [SMILES: CC(C)Cn1cc(-c2ccccc2NS(=O)(=O)C(C)C)c2ccc(-c3ccccc3)cc21]

### Step 1: 2-(1-isobutyl-6-phenyl-1H-indol-3-yl)aniline

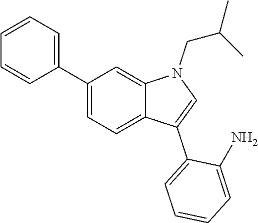 [SMILES: CC(C)Cn1cc(-c2ccccc2N)c2ccc(-c3ccccc3)cc21]

The title compound was prepared following the same general protocol as described for Step 5, Example 1, using 2-(4,4,5,5-tetramethyl-1,3,2-dioxaborolan-2-yl)aniline and 3-bromo-1-isobutyl-6-phenyl-1H-indole. ESI-MS (m/z): 341.04 [M+1]⁺.

### Step 2: N-(3-(1-isobutyl-6-phenyl-1H-indol-3-yl)phenyl)propane-2-sulfonamide

The title compound was prepared following the same general protocol as described for Step 2, Example 77, using 2-(1-isobutyl-6-phenyl-1H-indol-3-yl)aniline. ESI-MS (m/z): 447.81 [M+1]⁺.

### Example 82: dimethyl({2-[2-methyl-1-(2-methylpropyl)-6-(pyridin-4-yl)-1H-indol-3-yl]ethyl}sulfamoyl)amine (41)

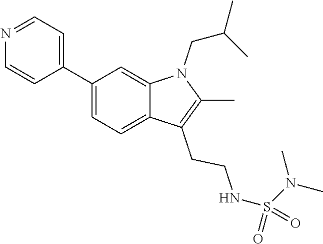 [SMILES: Cc1c(CCNS(=O)(=O)N(C)C)c2ccc(-c3ccncc3)cc2n1CC(C)C]

### Step 1: (Z)-3-(4-bromo-2-nitrophenyl)-4-ethoxy-4-hydroxybut-3-en-2-one

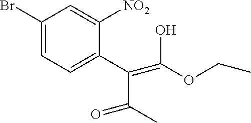 [SMILES: CCO/C(O)=C(\C(C)=O)c1ccc(Br)cc1[N+](=O)[O-]]

To a solution of 4-bromo-1-fluoro-2-nitrobenzene (6 mL, 48.7 mmol) in DMF (30 mL) in a 50° C. oil bath was added K₂CO₃ (14.8 g, 107.2 mmol), followed by addition of ethyl 3-oxobutanoate (7 mL, 53.6 mmol). The mixture was heated in a 50° C. oil bath overnight. The mixture was cooled to RT and diluted with EtOAc. The solution was washed with water, brine and dried over Na₂SO₄. The organic layer was concentrated in vacuo to obtain the title compound with no purification.

### Step 2: 1-(4-bromo-2-nitrophenyl)propan-2-one

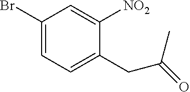

The crude from previous step was dissolved in AcOH (60 mL) and 50% H₂SO₄ (17 g) was added. The solution was refluxed overnight. The solution was concentrated in vacuo to obtain the crude, which was re-dissolved with EtOAc, washed with water, brine and dried over Na₂SO₄. The organic layer was concentrated in vacuo to obtain the title compound with no purification.

### Step 3: 6-bromo-2-methyl-1H-indole

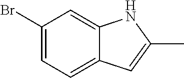

To the crude from the previous step dissolved in EtOH (100 mL) was added AcOH (34 mL, 584.5 mmol) followed by Zn (19.1 g, 292 mmol). The solution was heated in a 70° C. oil bath for 3h. The solution was cooled to RT, filtered through a celite pad and concentrated in vacuo. The crude was re-dissolved in EtOAc and washed with water sat'd NaHCO₃, brine, dried (Na₂SO₄) and filtered. The organic layer was concentrated in vacuo to obtain the title compound with no purification. ESI-MS (m/z): 209.82, 211.78 [M+1]⁺.

### Step 4: 6-bromo-1-isobutyl-2-methyl-1H-indole

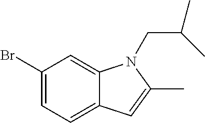

The title compound was prepared following the same general protocol as described for Step 1, Example 1, using 6-bromo-2-methyl-1H-indole. ESI-MS (m/z): 266.04, 267.98 [M+1]⁺.

### Step 5: 2-(6-bromo-1-isobutyl-2-methyl-1H-indol-3-yl)-2-oxoacetamide

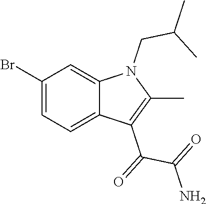 [SMILES: Cc1c(C(=O)C(N)=O)c2ccc(Br)cc2n1CC(C)C]

The title compound was prepared following the same general protocol as described for Step 2, Example 1, using 6-bromo-1-isobutyl-2-methyl-1H-indole. ESI-MS (m/z): 336.85, 338.77 [M+1]⁺.

### Step 6: 2-(6-bromo-1-isobutyl-2-methyl-1H-indol-3-yl)ethan-1-amine

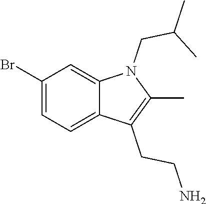 [SMILES: Cc1c(CCN)c2ccc(Br)cc2n1CC(C)C]

The title compound was prepared following the same general protocol as described for Step 3, Example 1, using 2-(6-bromo-1-isobutyl-2-methyl-1H-indol-3-yl)-2-oxoacetamide. ESI-MS (m/z): 308.87, 310.81 [M+1]⁺.

### Step 7: dimethyl({2-[2-methyl-1-(2-methylpropyl)-6-(pyridin-4-yl)-1H-indol-3-yl]ethyl}sulfamoyl)amine

The title compound was prepared following the same general protocol as described for Step 4, Example 1, using 2-(6-bromo-1-isobutyl-2-methyl-1H-indol-3-yl)ethan-1-amine. ESI-MS (m/z): 415.19 [M+1]⁺.

### Example 83: 1-(3-{2-[(dimethylsulfamoyl)amino]ethyl}-7-methyl-6-(pyridin-4-yl)-1H-indol-1-yl)-2-methylpropan-1-one (199)

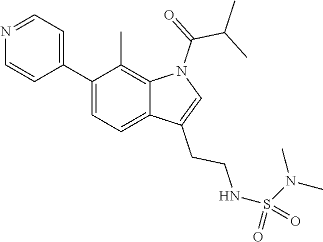 [SMILES: Cc1c(-c2ccncc2)ccc2c(CCNS(=O)(=O)N(C)C)cn(C(=O)C(C)C)c12]

### Step 1: methyl 2-(6-bromo-7-methyl-1H-indol-3-yl)-2-oxoacetate

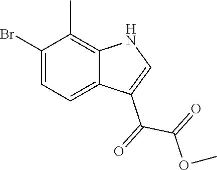 [SMILES: COC(=O)C(=O)c1c[nH]c2c(C)c(Br)ccc12]

The title compound was prepared following the same general protocol as described for Step 2, Example 1, using 6-bromo-7-methyl-1H-indole and MeOH. ESI-MS (m/z): 295.64, 297.62 [M+1]⁺.

### Step 2: 2-(6-bromo-7-methyl-1H-indol-3-yl)ethan-1-ol

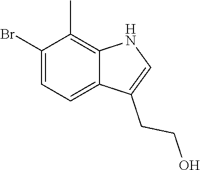

The title compound was prepared following the same general protocol as described for Step 3, Example 1, using methyl 2-(6-bromo-7-methyl-1H-indol-3-yl)-2-oxoacetate at RT. ESI-MS (m/z): 253.70, 255.73 [M+1]⁺.

### Step 3: tert-butyl (2-(6-bromo-7-methyl-1H-indol-3-yl)ethyl)(N,N-dimethylsulfamoyl)carbamate

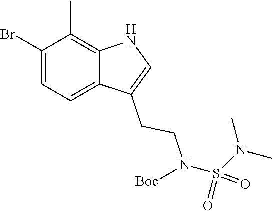 [SMILES: Cc1c(Br)ccc2c(CCN(C(=O)OC(C)(C)C)S(=O)(=O)N(C)C)c[nH]c12]

The title compound was prepared following the same general protocol as described for Step 7, Example 37, using tert-butyl (N,N-dimethylsulfamoyl)carbamate and 2-(6-bromo-7-methyl-1H-indol-3-yl)ethan-1-ol. ESI-MS (m/z): 459.65, 461.72 [M+1]⁺.

### Step 4: tert-butyl (2-(6-bromo-1-isobutyryl-7-methyl-1H-indol-3-yl)ethyl)(N,N-dimethylsulfamoyl)carbamate

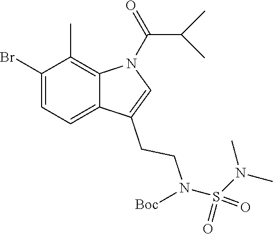 [SMILES: Cc1c(Br)ccc2c(CCN(C(=O)OC(C)(C)C)S(=O)(=O)N(C)C)cn(C(=O)C(C)C)c12]

The title compound was prepared following the same general protocol as described for Example 34, using tert-butyl (2-(6-bromo-7-methyl-1H-indol-3-yl)ethyl)(N,N-dimethylsulfamoyl)carbamate and isobutyryl chloride. ESI-MS (m/z): 529.91, 531.81 [M+1]⁺.

### Step 5: tert-butyl (N,N-dimethylsulfamoyl)(2-(1-isobutyryl-7-methyl-6-(pyridin-4-yl)-1H-indol-3-yl)ethyl)carbamate

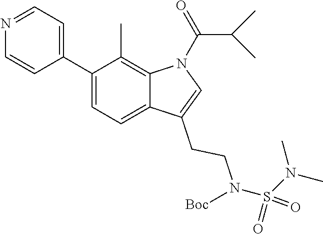 [SMILES: Cc1c(-c2ccncc2)ccc2c(CCN(C(=O)OC(C)(C)C)S(=O)(=O)N(C)C)cn(C(=O)C(C)C)c12]

The title compound was prepared following the same general protocol as described for Step 5, Example 1, using tert-butyl (2-(6-bromo-1-isobutyryl-7-methyl-1H-indol-3-yl)ethyl)(N,N-dimethylsulfamoyl)carbamate. ESI-MS (m/z): 528.90 [M+1]⁺.

### Step 6: 1-(3-{2-[(dimethylsulfamoyl)amino]ethyl}-7-methyl-6-(pyridin-4-yl)-1H-indol-1-yl)-2-methylpropan-1-one

The title compound was prepared following the same general protocol as described for Example 25, using tert-butyl (2-(6-bromo-1-isobutyryl-7-methyl-1H-indol-3-yl)ethyl)(N,N-dimethylsulfamoyl)carbamate. ESI-MS (m/z): 428.95 [M+1]⁺.

### Example 84: dimethyl({2-[7-methyl-1-(2-methylpropyl)-6-(pyridin-4-yl)-1H-indol-3-yl]ethyl}sulfamoyl)amine (200)

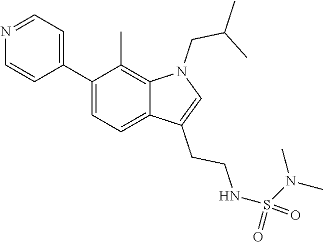 [SMILES: Cc1c(-c2ccncc2)ccc2c(CCNS(=O)(=O)N(C)C)cn(CC(C)C)c12]

### Step 1: tert-butyl (2-(6-bromo-1-isobutyl-7-methyl-1H-indol-3-yl)ethyl)(N,N-dimethylsulfamoyl)carbamate

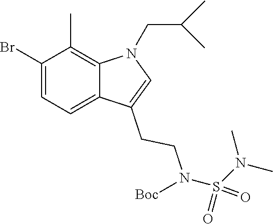 [SMILES: Cc1c(Br)ccc2c(CCN(C(=O)OC(C)(C)C)S(=O)(=O)N(C)C)cn(CC(C)C)c12]

The title compound was prepared following the same general protocol as described for Step 1, Example 1, using tert-butyl (2-(6-bromo-7-methyl-1H-indol-3-yl)ethyl)(N,N-dimethylsulfamoyl)carbamate. ESI-MS (m/z): 515.76, 517.70 [M+1]⁺.

### Step 2: tert-butyl (N,N-dimethylsulfamoyl)(2-(1-isobutyl-7-methyl-6-(pyridin-4-yl)-1H-indol-3-yl)ethyl)carbamate

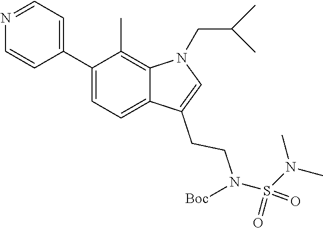 [SMILES: Cc1c(-c2ccncc2)ccc2c(CCN(C(=O)OC(C)(C)C)S(=O)(=O)N(C)C)cn(CC(C)C)c12]

The title compound was prepared following the same general protocol as described for Step 5, Example 1, using tert-butyl (2-(6-bromo-1-isobutyl-7-methyl-1H-indol-3-yl)ethyl)(N,N-dimethylsulfamoyl)carbamate. ESI-MS (m/z): 517.69 [M+1]⁺.

### Step 3: dimethyl({2-[7-methyl-1-(2-methylpropyl)-6-(pyridin-4-yl)-1H-indol-3-yl]ethyl}sulfamoyl)amine

The title compound was prepared following the same general protocol as described for Example 25, using tert-butyl (N,N-dimethylsulfamoyl)(2-(1-isobutyl-7-methyl-6-(pyridin-4-yl)-1H-indol-3-yl)ethyl)carbamate. ESI-MS (m/z): 414.99 [M+1]⁺.

### Example 85: dimethyl({2-[4-methyl-1-(2-methylpropyl)-6-(pyridin-4-yl)-1H-indol-3-yl]ethyl}sulfamoyl)amine (202)

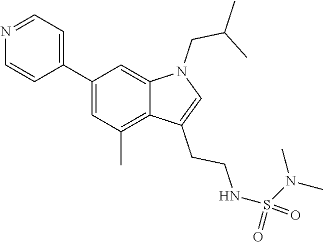 [SMILES: Cc1cc(-c2ccncc2)cc2c1c(CCNS(=O)(=O)N(C)C)cn2CC(C)C]

### Step 1: (E)-2-(4-bromo-2-methyl-6-nitrophenyl)-N,N-dimethylethen-1-amine

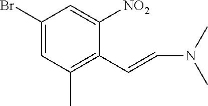 [SMILES: Cc1cc(Br)cc([N+](=O)[O-])c1/C=C/N(C)C]

The title compound was prepared following the same general protocol as described for Step 1, Example 63, using 5-bromo-1,2-dimethyl-3-nitrobenzene. ESI-MS (m/z): 284.65, 286.71 [M+1]^{m}.

### Step 2: 6-bromo-4-methyl-1H-indole

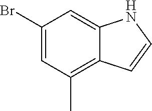

The title compound was prepared following the same general protocol as described for Step 2, Example 67, using (E)-2-(4-bromo-2-methyl-6-nitrophenyl)-N,N-dimethylethen-1-amine. ESI-MS (m/z): 209.77, 211.76 [M+1]⁺.

### Step 3: 6-bromo-1-isobutyl-4-methyl-1H-indole

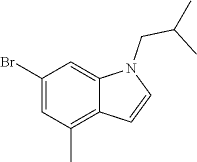

The title compound was prepared following the same general protocol as described for Step 1, Example 1, using 6-bromo-4-methyl-1H-indole. ESI-MS (m/z): 265.80, 267.80 [M+1].

### Step 4: 2-(6-bromo-1-isobutyl-4-methyl-1H-indol-3-yl)-2-oxoacetamide

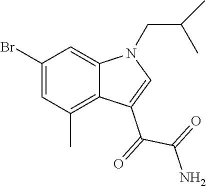 [SMILES: Cc1cc(Br)cc2c1c(C(=O)C(N)=O)cn2CC(C)C]

The title compound was prepared following the same general protocol as described for Step 2, Example 1, using 6-bromo-1-isobutyl-4-methyl-1H-indole. ESI-MS (m/z): 336.61, 338.60 [M+1]⁺.

### Step 5: 2-(6-bromo-1-isobutyl-4-methyl-1H-indol-3-yl)ethan-1-amine

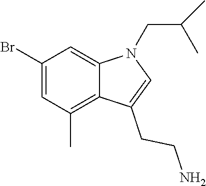 [SMILES: Cc1cc(Br)cc2c1c(CCN)cn2CC(C)C]

The title compound was prepared following the same general protocol as described for Step 3, Example 1, using 2-(6-bromo-1-isobutyl-4-methyl-1H-indol-3-yl)-2-oxoacetamide. ESI-MS (m/z): 308.70, 310.71 [M+1]⁺.

### Step 6: ({2-[6-bromo-4-methyl-1-(2-methylpropyl)-1H-indol-3-yl]ethyl}sulfamoyl)dimethylamine

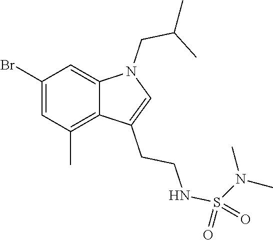 [SMILES: Cc1cc(Br)cc2c1c(CCNS(=O)(=O)N(C)C)cn2CC(C)C]

The title compound was prepared following the same general protocol as described for Step 4, Example 1, using 2-(6-bromo-1-isobutyl-4-methyl-1H-indol-3-yl)ethan-1-amine. ESI-MS (m/z): 415.81, 417.86 [M+1]⁺.

### Step 7: dimethyl({2-[4-methyl-1-(2-methylpropyl)-6-(pyridin-4-yl)-1H-indol-3-yl]ethyl}sulfamoyl)amine

The title compound was prepared following the same general protocol as described for Step 5, Example 1, using ({2-[6-bromo-4-methyl-1-(2-methylpropyl)-1H-indol-3-yl]ethyl}sulfamoyl)dimethylamine. ESI-MS (m/z): 415.01 [M+1]⁺.

### Example 86: dimethyl({2-[1-(2-methylpropyl)-6-(pyridin-4-yl)-1H-pyrrolo[2,3-b]pyridin-3-yl]ethyl}sulfamoyl)amine (203)

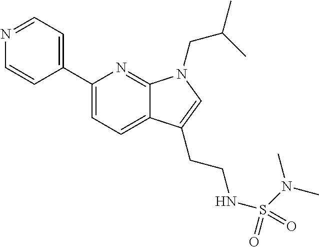 [SMILES: CC(C)Cn1cc(CCNS(=O)(=O)N(C)C)c2ccc(-c3ccncc3)nc21]

### Step 1: 6-bromo-1-isobutyl-1H-pyrrolo[2,3-b]pyridine

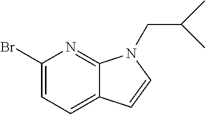

The title compound was prepared following the same general protocol as described for Step 1, Example 1, using 6-bromo-1H-pyrrolo[2,3-b]pyridine. ESI-MS (m/z): 252.75, 254.72 [M+1]⁺.

### Step 2: 2-(6-bromo-1-isobutyl-1H-pyrrolo[2,3-b]pyridin-3-yl)-2-oxoacetamide

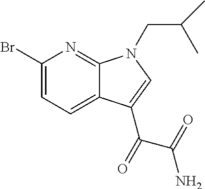 [SMILES: CC(C)Cn1cc(C(=O)C(N)=O)c2ccc(Br)nc21]

The title compound was prepared following the same general protocol as described for Step 2, Example 1, using 6-bromo-1-isobutyl-1H-pyrrolo[2,3-b]pyridine. ESI-MS (m/z): 323.75, 325.74 [M+1]⁺.

### Step 3: 2-(6-bromo-1-isobutyl-1H-pyrrolo[2,3-b]pyridin-3-yl)ethan-1-amine

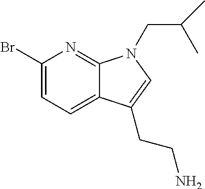 [SMILES: CC(C)Cn1cc(CCN)c2ccc(Br)nc21]

The title compound was prepared following the same general protocol as described for Step 3, Example 1, using 2-(6-bromo-1-isobutyl-1H-pyrrolo[2,3-b]pyridin-3-yl)-2-oxoacetamide. ESI-MS (m/z): 295.76, 297.65 [M+1]⁺.

### Step 4: ({2-[6-bromo-1-(2-methylpropyl)-1H-pyrrolo[2,3-b]pyridin-3-yl]ethyl}sulfamoyl)dimethylamine

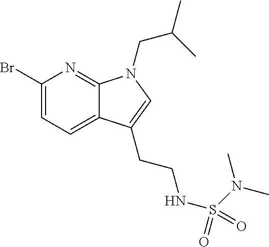 [SMILES: CC(C)Cn1cc(CCNS(=O)(=O)N(C)C)c2ccc(Br)nc21]

The title compound was prepared following the same general protocol as described for Step 4, Example 1, using 2-(6-bromo-1-isobutyl-1H-pyrrolo[2,3-b]pyridin-3-yl)ethan-1-amine. ESI-MS (m/z): 402.67, 404.65 [M+1]⁺.

### Step 5: dimethyl({2-[1-(2-methylpropyl)-6-(pyridin-4-yl)-1H-pyrrolo[2,3-b]pyridin-3-yl]ethyl}sulfamoyl)amine

The title compound was prepared following the same general protocol as described for Step 5, Example 1, using ({2-[6-bromo-1-(2-methylpropyl)-1H-pyrrolo[2,3-b]pyridin-3-yl]ethyl}sulfamoyl)dimethylamine. ESI-MS (m/z): 402.12 [M+1]⁺.

### Example 87: dimethyl({2-[3-(2-methylpropyl)-5-(pyridin-4-yl)-1H-indol-1-yl]ethyl}sulfamoyl)amine (23)

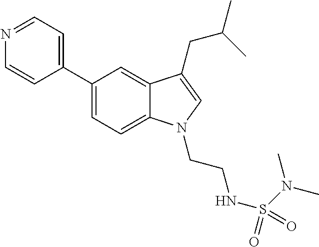 [SMILES: CC(C)Cc1cn(CCNS(=O)(=O)N(C)C)c2ccc(-c3ccncc3)cc12]

### Step 1: 1-(5-bromo-1H-indol-3-yl)-2-methylpropan-1-one

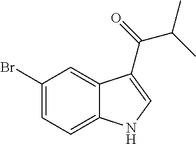

To a solution of 5-bromo-1H-indole (1.0 g, 5.1 mmol) in DCM (20 mL) at 0° C. was slowly added Et₂AlCl (7.7 mL, 1M in hexane, 7.65 mmol). After 30 min, isobutyryl chloride (0.8 mL, 7.65 mmol) in DCM (20 mL) was added. The solution was stirred at 0° C. and monitored for reaction completion by reverse phase analytical HPLC. After 2h, the solution was diluted with DCM, and quenched with slow addition of water. The aqueous layer was extracted with DCM. The combined organic layers washed with brine and dried over Na₂SO₄. The organic layer was filtered through celite and concentrated to obtain the title compound with no purification. ESI-MS (m/z): 265.88, 267.80 [M+1]⁺.

### Step 2: 5-bromo-3-isobutyl-1H-indole

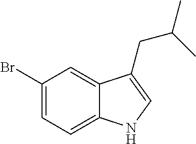

The title compound was prepared following the same general protocol as described for Step 3, Example 1, using 1-(5-bromo-1H-indol-3-yl)-2-methylpropan-1-one. ESI-MS (m/z): 251.93, 253.96 [M+1]⁺.

### Step 3: tert-butyl (2-(5-bromo-3-isobutyl-1H-indol-1-yl)ethyl)carbamate

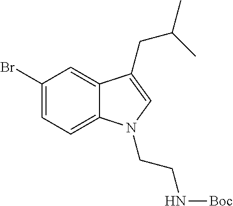 [SMILES: CC(C)Cc1cn(CCNC(=O)OC(C)(C)C)c2ccc(Br)cc12]

To a solution of 5-bromo-3-isobutyl-1H-indole (0.798 g, 3.16 mmol) and KOH (0.53 g, 9.48 mmol) in DMSO at 40° C. was added portionwise 2-((tert-butoxycarbonyl)amino)ethyl methanesulfonate (7.9 g, 7.9 mmol) in DMSO (4 mL). The solution was stirred at 40° C. overnight. The solution was cooled to RT and water was added and then extracted with DCM. The solvent was removed to obtain the crude which was purified by chromatography on silica gel (EtOAc/hex) to afford the title compound. ESI-MS (m/z): 394.74, 396.72 [M+1]⁺.

### Step 4: tert-butyl (2-(3-isobutyl-5-(pyridin-4-yl)-1H-indol-1-yl)ethyl)carbamate

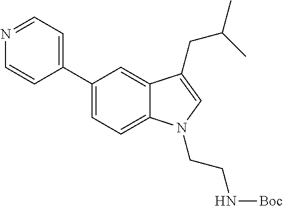 [SMILES: CC(C)Cc1cn(CCNC(=O)OC(C)(C)C)c2ccc(-c3ccncc3)cc12]

The title compound was prepared following the same general protocol as described for Step 5, Example 1, using tert-butyl (2-(5-bromo-3-isobutyl-1H-indol-1-yl)ethyl)carbamate. ESI-MS (m/z): 394.12 [M+1]⁺.

### Step 5: 2-(3-isobutyl-5-(pyridin-4-yl)-1H-indol-1-yl)ethan-1-amine

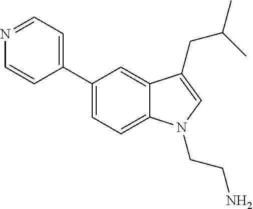 [SMILES: CC(C)Cc1cn(CCN)c2ccc(-c3ccncc3)cc12]

The title compound was prepared following the same general protocol as described for Example 25, using tert-butyl (2-(3-isobutyl-5-(pyridin-4-yl)-1H-indol-1-yl)ethyl)carbamate. ESI-MS (m/z): 293.98 [M+1]⁺.

### Step 6: dimethyl({2-[3-(2-methylpropyl)-5-(pyridin-4-yl)-1H-indol-1-yl]ethyl}sulfamoyl)amine

The title compound was prepared following the same general protocol as described for Step 4, Example 1, using 2-(3-isobutyl-5-(pyridin-4-yl)-1H-indol-1-yl)ethan-1-amine. ESI-MS (m/z): 401.05 [M+1]⁺.

### Example 88: dimethyl({2-[3-(2-methylpropyl)-5-(pyridin-4-yl)-1H-pyrrolo[2,3-b]pyridin-1-yl]ethyl}sulfamoyl)amine

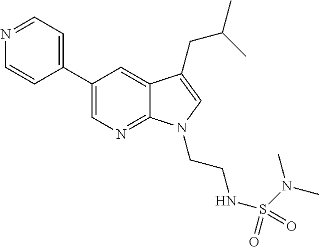 [SMILES: CC(C)Cc1cn(CCNS(=O)(=O)N(C)C)c2ncc(-c3ccncc3)cc12]

### Step 1: 1-(5-bromo-1H-pyrrolo[2,3-b]pyridin-3-yl)-2-methylpropan-1-one

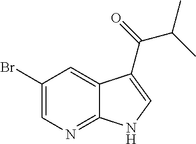

The title compound was prepared following the same general protocol as described for Step 1, Example 87, using AlCl₃ and 5-bromo-1H-pyrrolo[2,3-b]pyridine. ESI-MS (m/z): 267.02, 268.96 [M+1]⁺. ¹HNMR (CDCl₃, 400 MHz) δ 11.56 (s, 1H), 8.90 (d, J=4.0 Hz, 1H), 8.47 (d, J=4.0 Hz, 1H), 8.06 (d, J=4.0 Hz, 1H), 3.34 (m, 1H), 1.28 (d, J=8.0 Hz, 6H)

### Step 2: 5-bromo-3-isobutyl-1H-pyrrolo[2,3-b]pyridine

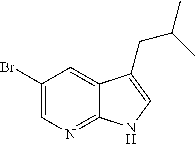

To a solution of 1-(5-bromo-1H-pyrrolo[2,3-b]pyridin-3-yl)-2-methylpropan-1-one (1.879 g, 7.03 mmol) in TFA (20 mL) was slowly added triethylsilane. The solution was heated in a 80° C. oil bath for 1h and the completion of reaction was monitored by reverse phase analytical HPLC. The solution was cooled to RT and concentrated in vacuo. The crude was dissolved with EtOAc, washed with water, sat'd NaHCO₃, brine, dried (MgSO₄) and filtered. The solvent was removed in vacuo to obtain the crude which was purified by chromatography on silica gel (EtOAc/hex) to afford the title compound. ESI-MS (m/z): 252.99, 254.86 [M+1]⁺.

### Step 3: tert-butyl (2-(5-bromo-3-isobutyl-1H-pyrrolo[2,3-b]pyridin-1-yl)ethyl)carbamate

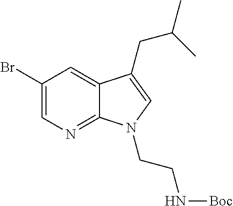 [SMILES: CC(C)Cc1cn(CCNC(=O)OC(C)(C)C)c2ncc(Br)cc12]

The title compound was prepared following the same general protocol as described for Example 34, using 5-bromo-3-isobutyl-1H-pyrrolo[2,3-b]pyridine and tert-butyl (2-bromoethyl)carbamate. ESI-MS (m/z): 396.05, 397.87 [M+1]⁺.

### Step 4: tert-butyl (2-(3-isobutyl-5-(pyridin-4-yl)-1H-pyrrolo[2,3-b]pyridin-1-yl)ethyl)carbamate

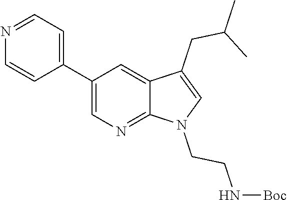 [SMILES: CC(C)Cc1cn(CCNC(=O)OC(C)(C)C)c2ncc(-c3ccncc3)cc12]

The title compound was prepared following the same general protocol as described for Step 5, Example 1, using tert-butyl (2-(5-bromo-3-isobutyl-1H-pyrrolo[2,3-b]pyridin-1-yl)ethyl)carbamate. ESI-MS (m/z): 395.15 [M+1]⁺.

### Step 5: 2-(3-isobutyl-5-(pyridin-4-yl)-1H-pyrrolo[2,3-b]pyridin-1-yl)ethan-1-amine

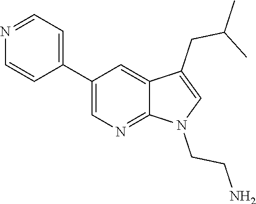 [SMILES: CC(C)Cc1cn(CCN)c2ncc(-c3ccncc3)cc12]

The title compound was prepared following the same general protocol as described for Example 25, using tert-butyl (2-(3-isobutyl-5-(pyridin-4-yl)-1H-pyrrolo[2,3-b]pyridin-1-yl)ethyl)carbamate. ESI-MS (m/z): 294.99, [M+1]⁺.

### Step 6: dimethyl({2-[3-(2-methylpropyl)-5-(pyridin-4-yl)-1H-pyrrolo[2,3-b]pyridin-1-yl]ethyl}sulfamoyl)amine

The title compound was prepared following the same general protocol as described for Step 4, Example 1, using 2-(3-isobutyl-5-(pyridin-4-yl)-1H-pyrrolo[2,3-b]pyridin-1-yl)ethan-1-amine. ESI-MS (m/z): 402.16 [M+1]⁺.

### Example 89: N-(2-(3-isobutyl-5-(pyridin-4-yl)-1H-pyrrolo[2,3-b]pyridin-1-yl)ethyl)cyclopropanesulfonamide (365)

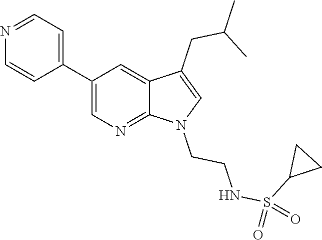 [SMILES: CC(C)Cc1cn(CCNS(=O)(=O)C2CC2)c2ncc(-c3ccncc3)cc12]

The title compound was prepared following the same general protocol as described for Step 4, Example 1, using 2-(3-isobutyl-5-(pyridin-4-yl)-1H-pyrrolo[2,3-b]pyridin-1-yl)ethan-1-amine and cyclopropanesulfonyl chloride. ESI-MS (m/z): 399.16 [M+1]⁺.

### Example 90: dimethyl[methyl({2-[3-(2-methylpropyl)-5-(pyridin-4-yl)-1H-pyrrolo[2,3-b]pyridin-1-yl]ethyl})sulfamoyl]amine (125)

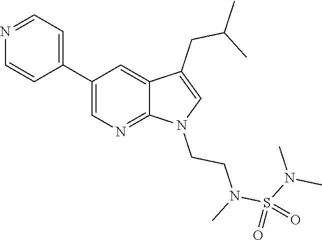 [SMILES: CC(C)Cc1cn(CCN(C)S(=O)(=O)N(C)C)c2ncc(-c3ccncc3)cc12]

The title compound was prepared following the same general protocol as described for Example 34, using dimethyl({2-[3-(2-methylpropyl)-5-(pyridin-4-yl)-1H-pyrrolo[2,3-b]pyridin-1-yl]ethyl}sulfamoyl)amine. ESI-MS (m/z): 416.05 [M+1]⁺.

### Example 91: N-(2-(3-isobutyl-5-(pyridin-4-yl)-1H-pyrrolo[2,3-b]pyridin-1-yl)ethyl)-N-methylcyclopropanesulfonamide (126)

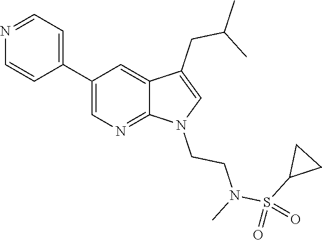 [SMILES: CC(C)Cc1cn(CCN(C)S(=O)(=O)C2CC2)c2ncc(-c3ccncc3)cc12]

The title compound was prepared following the same general protocol as described for Example 34, using N-(2-(3-isobutyl-5-(pyridin-4-yl)-1H-pyrrolo[2,3-b]pyridin-1-yl)ethyl)cyclopropanesulfonamide. ESI-MS (m/z): 413.03 [M+1]⁺.

### Example 92: dimethyl[methyl({2-[3-(2-methylpropyl)-5-(pyridin-4-yl)-1H-pyrrolo[2,3-b]pyridin-1-yl]ethyl})sulfamoyl]amine (191)

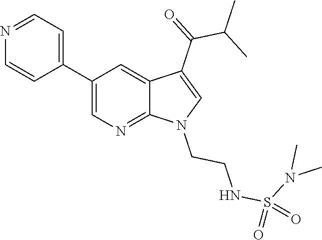 [SMILES: CC(C)C(=O)c1cn(CCNS(=O)(=O)N(C)C)c2ncc(-c3ccncc3)cc12]

### Step 1: 1-(1-(2-aminoethyl)-5-bromo-1H-pyrrolo[2,3-b]pyridin-3-yl)-2-methylpropan-1-one

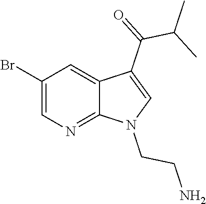 [SMILES: CC(C)C(=O)c1cn(CCN)c2ncc(Br)cc12]

To a solution of 1-(5-bromo-1H-pyrrolo[2,3-b]pyridin-3-yl)-2-methylpropan-1-one (1.6 g, 5.91 mmol) in toluene (50 mL) was added NaOH (1.65 g, 41.37 mmol), tetrabutylammonium hydrosulfate (0.2 g, 1.182 mmol) and 2-chloroethan-1-amine HCl salt (1.37 g, 11.82 mmol). The mixture was heated in a 120° C. oil bath overnight. The solution was cooled to RT and concentrated in vacuo. The crude was dissolved in EtOAc, washed with water, sat'd NaHCO₃, brine, dried (MgSO₄) and filtered. The solvent was removed in vacuo to obtain the crude which was purified by chromatography on silica gel (EtOAc/hex) to afford the title compound. ESI-MS (m/z): 309.86, 311.85 [M+1]⁺.

### Step 2: 1-(5-bromo-1-{2-[(dimethylsulfamoyl)amino]ethyl}-1H-pyrrolo[2,3-b]pyridin-3-yl)-2-methylpropan-1-one

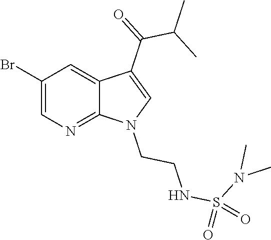 [SMILES: CC(C)C(=O)c1cn(CCNS(=O)(=O)N(C)C)c2ncc(Br)cc12]

The title compound was prepared following the same general protocol as described for Step 4, Example 1, using 1-(1-(2-aminoethyl)-5-bromo-1H-pyrrolo[2,3-b]pyridin-3-yl)-2-methylpropan-1-one. ESI-MS (m/z): 416.83, 418.80 [M+1]⁺.

### Step 3: dimethyl[methyl({2-[3-(2-methylpropyl)-5-(pyridin-4-yl)-1H-pyrrolo[2,3-b]pyridin-1-yl]ethyl})sulfamoyl]amine

The title compound was prepared following the same general protocol as described for Step 5, Example 1, using 1-(5-bromo-1-{2-[(dimethylsulfamoyl)amino]ethyl}-1H-pyrrolo[2,3-b]pyridin-3-yl)-2-methylpropan-1-one. ESI-MS (m/z): 415.98 [M+1]⁺.

### Example 93: 1-[5-(2-chlorophenyl)-1-{2-[(dimethylsulfamoyl)amino]ethyl}-1H-pyrrolo[2,3-b]pyridin-3-yl]-2-methylpropan-1-one (192)

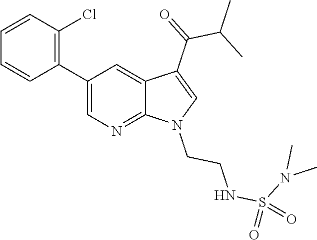 [SMILES: CC(C)C(=O)c1cn(CCNS(=O)(=O)N(C)C)c2ncc(-c3ccccc3Cl)cc12]

The title compound was prepared following the same general protocol as described for Step 5, Example 1, using 1-(5-bromo-1-{2-[(dimethylsulfamoyl)amino]ethyl}-1H-pyrrolo[2,3-b]pyridin-3-yl)-2-methylpropan-1-One and (2-chlorophenyl)boronic acid. ESI-MS (m/z): 448.82 [M+1]⁺.

### Example 96: dimethyl({2-[6-(pyridin-4-yl)-1-(3,3,3-trifluoropropyl)-1H-indol-3-yl]ethyl}sulfamoyl)amine (179)

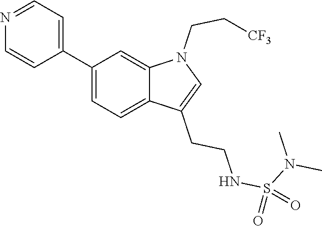 [SMILES: CN(C)S(=O)(=O)NCCc1cn(CCC(F)(F)F)c2cc(-c3ccncc3)ccc12]

### Step 1: methyl 2-(6-bromo-1-(3,3,3-trifluoropropyl)-1H-indol-3-yl)-2-oxoacetate

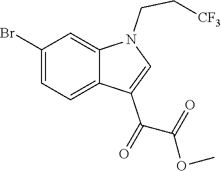 [SMILES: COC(=O)C(=O)c1cn(CCC(F)(F)F)c2cc(Br)ccc12]

The title compound was prepared following the same general protocol as described for Step 1, Example 1, using methyl 2-(6-bromo-1H-indol-3-yl)-2-oxoacetate and 1,1,1-trifluoro-3-iodopropane. ESI-MS (m/z): 377.73, 379.73 [M+1]⁺.

### Step 2: 2-(6-bromo-1-(3,3,3-trifluoropropyl)-1H-indol-3-yl)ethan-1-ol

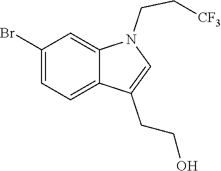 [SMILES: OCCc1cn(CCC(F)(F)F)c2cc(Br)ccc12]

The title compound was prepared following the same general protocol as described for Step 3, Example 1, using methyl 2-(6-bromo-1-(3,3,3-trifluoropropyl)-1H-indol-3-yl)-2-oxoacetate and 2-(6-bromo-1-(3,3,3-trifluoropropyl)-1H-indol-3-yl)-2-oxoacetic acid-methane and RT. ESI-MS (m/z): 335.80, 337.78 [M+1]⁺.

### Step 3: tert-butyl (2-(6-bromo-1-(3,3,3-trifluoropropyl)-1H-indol-3-yl)ethyl)(N,N-dimethylsulfamoyl)carbamate

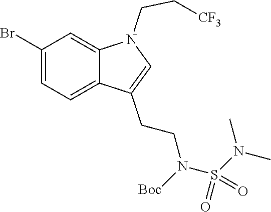 [SMILES: CN(C)S(=O)(=O)N(CCc1cn(CCC(F)(F)F)c2cc(Br)ccc12)C(=O)OC(C)(C)C]

The title compound was prepared following the same general protocol as described for Step 7, Example 37, using tert-butyl (N,N-dimethylsulfamoyl)carbamate and 2-(6-bromo-1-(3,3,3-trifluoropropyl)-1H-indol-3-yl)ethan-1-ol. ESI-MS (m/z): 543.44, 545.65 [M+1]⁺.

### Step 4: tert-butyl (N,N-dimethylsulfamoyl)(2-(6-(pyridin-4-yl)-1-(3,3,3-trifluoropropyl)-1H-indol-3-yl)ethyl)carbamate

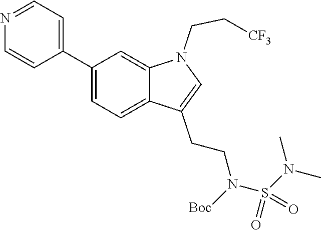 [SMILES: CN(C)S(=O)(=O)N(CCc1cn(CCC(F)(F)F)c2cc(-c3ccncc3)ccc12)C(=O)OC(C)(C)C]

The title compound was prepared following the same general protocol as described for Step 5, Example 1, using tert-butyl (2-(6-bromo-1-(3,3,3-trifluoropropyl)-1H-indol-3-yl)ethyl)(N,N-dimethylsulfamoyl)carbamate. ESI-MS (m/z): 540.92 [M+1]⁺.

### Step 5: dimethyl({2-[6-(pyridin-4-yl)-1-(3,3,3-trifluoropropyl)-1H-indol-3-yl]ethyl}sulfamoyl)amine

The title compound was prepared following the same general protocol as described for Example 25, using tert-butyl (N,N-dimethylsulfamoyl)(2-(6-(pyridin-4-yl)-1-(3,3,3-trifluoropropyl)-1H-indol-3-yl)ethyl)carbamate. ESI-MS (m/z): 440.94 [M+1]⁺.

### Example 98: dimethyl({2-methyl-2-[1-(2-methylpropyl)-6-(pyridin-4-yl)-1H-indol-3-yl]propyl}sulfamoyl)amine (177)

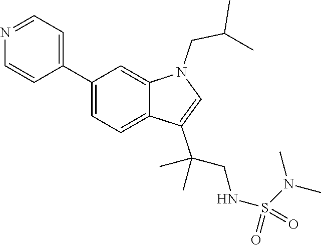 [SMILES: CC(C)Cn1cc(C(C)(C)CNS(=O)(=O)N(C)C)c2ccc(-c3ccncc3)cc21]

### Step 1: 1-(6-bromo-1-isobutyl-1H-indol-3-yl)-N,N-dimethylmethanamine

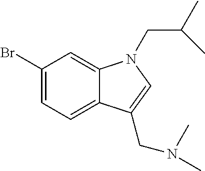 [SMILES: CC(C)Cn1cc(CN(C)C)c2ccc(Br)cc21]

The title compound was prepared following the same general protocol as described for Step 1, Example 97, using 6-bromo-1-isobutyl-1H-indole. ESI-MS (m/z): 308.48, 310.51 [M+1]⁺.

### Step 2: 1-(6-bromo-1-isobutyl-1H-indol-3-yl)-N,N,N-trimethylmethanaminium

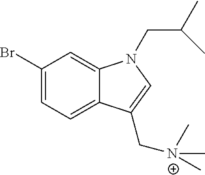 [SMILES: CC(C)Cn1cc(C[N+](C)(C)C)c2ccc(Br)cc21]

To a solution of 1-(6-bromo-1-isobutyl-1H-indol-3-yl)-N,N-dimethylmethanamine (5.49 g, 13.99 mmol) in EtOH (25 mL) was added Mel (1.8 mL, 27.98 mmol). The completion of the reaction was monitored by reverse phase analytical HPLC. The solution was concentrated in vacuo to obtain the title compound with no further purification. ESI-MS (m/z): 322.52, 324.58 [M+1]⁺.

### Step 3: 2-(6-bromo-1-isobutyl-1H-indol-3-yl)acetonitrile

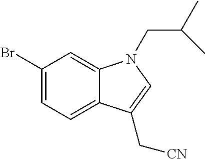 [SMILES: CC(C)Cn1cc(CC#N)c2ccc(Br)cc21]

A mixture of 1-(6-bromo-1-isobutyl-1H-indol-3-yl)-N,N,N-trimethylmethanaminium obtained from previous step (13.99 mmol) and NaCN (3.42 g, 69.95 mmol) in DMF (35 mL) and water (7 mL) was heated in a 120° C. oil bath overnight. The mixture was cooled to RT and concentrated in vacuo. The crude was dissolved in EtOAc and washed with water, sat'd NaHCO₃, brine, dried (Na₂SO₄,) and filtered. The solvent was removed in vacuo to obtain the crude which was purified by chromatography on silica gel (EtOAc/hex) to afford the title compound. ESI-MS (m/z): 263.75, 265.78 [M+1]⁺.

### Step 4: 2-(6-bromo-1-isobutyl-1H-indol-3-yl)-2-methylpropanenitrile

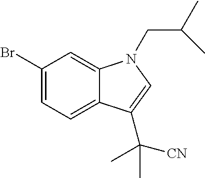 [SMILES: CC(C)Cn1cc(C(C)(C)C#N)c2ccc(Br)cc21]

To a solution of 2-(6-bromo-1-isobutyl-1H-indol-3-yl)acetonitrile (0.3 g, 1.03 mmol) in THF (5 mL) at −78° C. under argon was added LiHMDS (3.1 mL, 1M, 3.09 mmol) dropwise. After 30 min, Mel (0.13 mL, 2.06 mmol) was added and the solution was gradually warm to RT. The completion of reaction was monitored by reverse phase analytical HPLC. The solution was concentrated in vacuo. The crude was dissolved in EtOAc and washed with water, sat'd NaHCO₃, brine, dried (Na₂SO₄,) and filtered. The solvent was removed in vacuo to obtain the crude which was purified by chromatography on silica gel (EtOAc/hex) to afford the title compound. ESI-MS (m/z): 318.83, 320.91 [M+1]⁺.

### Step 5: 2-(1-isobutyl-6-(pyridin-4-yl)-1H-indol-3-yl)-2-methylpropanenitrile

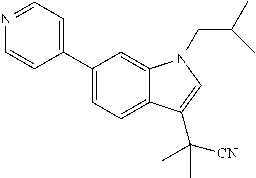 [SMILES: CC(C)Cn1cc(C(C)(C)C#N)c2ccc(-c3ccncc3)cc21]

The title compound was prepared following the same general protocol as described for Step 5, Example 1, using 2-(6-bromo-1-isobutyl-1H-indol-3-yl)-2-methylpropanenitrile. ESI-MS (m/z): 318.06 [M+1]⁺.

### Step 6: 2-(1-isobutyl-6-(pyridin-4-yl)-1H-indol-3-yl)-2-methylpropan-1-amine

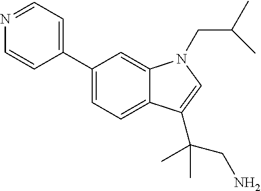 [SMILES: CC(C)Cn1cc(C(C)(C)CN)c2ccc(-c3ccncc3)cc21]

To a solution of 2-(1-isobutyl-6-(pyridin-4-yl)-1H-indol-3-yl)-2-methylpropanenitrile (0.1739 g, 0.548 mmol) in THF (5 mL) was added LAH (0.025 g, 0.658 mmol). The solution was heated at 70° C. under argon for 30 min. The reaction was cooled to RT, and sat'd NaHCO₃ and EtOAc were added. The organic layer was then separated and washed with brine and dried over Na₂SO₄ and filtered. The solvent was removed to obtain the title compound with no further purification. ESI-MS (m/z): 321.98 [M+1]⁺.

### Step 7: methyl({2-methyl-2-[1-(2-methylpropyl)-6-(pyridin-4-yl)-1H-indol-3-yl]propyl}sulfamoyl)amine

The title compound was prepared following the same general protocol as described for Step 4, Example 1, using 2-(1-isobutyl-6-(pyridin-4-yl)-1H-indol-3-yl)-2-methylpropan-1-amine. ESI-MS (m/z): 429.02 [M+1]⁺.

### Example 99: dimethyl[({1-[1-(2-methylpropyl)-6-(pyridin-4-yl)-1H-indol-3-yl]cyclopropyl}methyl)sulfamoyl]amine (178)

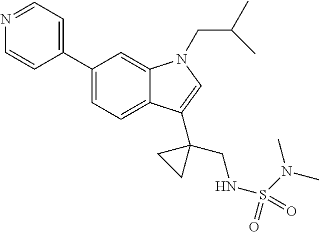 [SMILES: CC(C)Cn1cc(C2(CNS(=O)(=O)N(C)C)CC2)c2ccc(-c3ccncc3)cc21]

### Step 1: 1-(6-bromo-1-isobutyl-1H-indol-3-yl)cyclopropane-1-carbonitrile

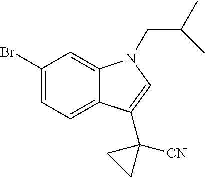 [SMILES: CC(C)Cn1cc(C2(C#N)CC2)c2ccc(Br)cc21]

The title compound was prepared following the same general protocol as described for Step 4, Example 98, using 1,2-dibromoethane. ESI-MS (m/z): 316.81, 318.80 [M+1]⁺.

### Step 2: 1-(1-isobutyl-6-(pyridin-4-yl)-1H-indol-3-yl)cyclopropane-1-carbonitrile

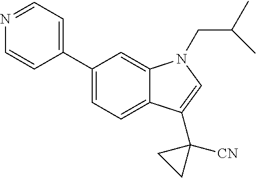 [SMILES: CC(C)Cn1cc(C2(C#N)CC2)c2ccc(-c3ccncc3)cc21]

The title compound was prepared following the same general protocol as described for Step 5, Example 1, using 1-(6-bromo-1-isobutyl-1H-indol-3-yl)cyclopropane-1-carbonitrile. ESI-MS (m/z): 316.07 [M+1]⁺.

### Step 3: (1-(1-isobutyl-6-(pyridin-4-yl)-1H-indol-3-yl)cyclopropyl)methanamine

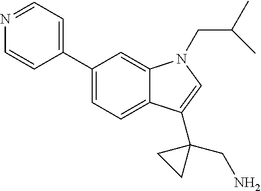 [SMILES: CC(C)Cn1cc(C2(CN)CC2)c2ccc(-c3ccncc3)cc21]

The title compound was prepared following the same general protocol as described for Step 6, Example 98, using 1-(1-isobutyl-6-(pyridin-4-yl)-1H-indol-3-yl)cyclopropane-1-carbonitrile. ESI-MS (m/z): 319.95 [M+1]⁺.

### Step 4: dimethyl[({1-[1-(2-methylpropyl)-6-(pyridin-4-yl)-1H-indol-3-yl]cyclopropyl}methyl)sulfamoyl]amine

The title compound was prepared following the same general protocol as described for Step 4, Example 1, using (1-(1-isobutyl-6-(pyridin-4-yl)-1H-indol-3-yl)cyclopropyl)methanamine. ESI-MS (m/z): 427.13 [M+1]⁺.

### Example 100: 1-(3-{1-[(dimethylsulfamoyl)amino]ethyl}-6-(pyridin-4-yl)-1H-indol-1-yl)-2-methylpropan-1-one (180)

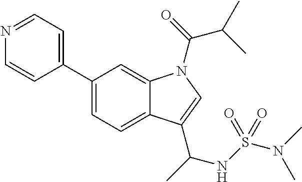 [SMILES: CC(C)C(=O)n1cc(C(C)NS(=O)(=O)N(C)C)c2ccc(-c3ccncc3)cc21]

### Step 1: 6-bromo-1-isobutyryl-1H-indole-3-carbaldehyde

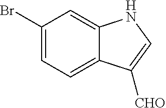 [SMILES: O=Cc1c[nH]c2cc(Br)ccc12]

To a solution of POCl₃ (4.6 mL, 49.7 mmol) in DMF (50 mL) at 0° C. was added 6-bromo indole (7.5 g, 38.3 mmol) in DMF (40 mL). The solution was then stirred at RT for 1h. The solution was poured into ice water, and adjusted to pH-9 by addition of KOH (5M) and then extracted with EtOAc. The organic layers were combined and washed with brine, dried over Na₂SO₄, filtered and concentrated in vacuo to obtain the title compound with no further purification. ESI-MS (m/z): 223.65, 225.68 [M+1]⁺.

### Step 2: 6-bromo-1H-indole-3-carbaldehyde

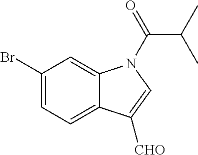 [SMILES: CC(C)C(=O)n1cc(C=O)c2ccc(Br)cc21]

The title compound was prepared following the same general protocol as described for Example 34, using 6-bromo-1H-indole-3-carbaldehyde and isobutyryl chloride. ESI-MS (m/z): 293.78, 295.65 [M+1]⁺.

### Step 3: 1-(6-bromo-3-(1-hydroxyethyl)-1H-indol-1-yl)-2-methylpropan-1-one

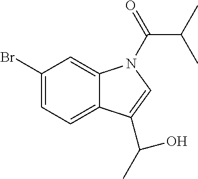 [SMILES: CC(C)C(=O)n1cc(C(C)O)c2ccc(Br)cc21]

To a solution of 6-bromo-1-isobutyryl-1H-indole-3-carbaldehyde (0.484 g, 1.645 mmol) in THF (8 mL) at 0° C. was added dropwise MeMgBr (0.6 mL, 3M in ether, 1.81 mmol). The solution was stirred for 30 min and then quenched by the addition of sat'd NH₄Cl and diluted with EtOAc. The organic layer was washed with brine, dried (Na₂SO₄) and filtered. The filtrate was concentrated in vacuo to obtain the title compound with no further purification. ESI-MS (m/z): 309.64, 311.80 [M+1]⁺.

### Step 4: tert-butyl (1-(6-bromo-1-isobutyryl-1H-indol-3-yl)ethyl)(N,N-dimethylsulfamoyl)carbamate

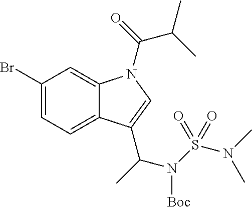 [SMILES: CC(C)C(=O)n1cc(C(C)N(C(=O)OC(C)(C)C)S(=O)(=O)N(C)C)c2ccc(Br)cc21]

The title compound was prepared following the same general protocol as described for Step 7, Example 37, using tert-butyl (N,N-dimethylsulfamoyl)carbamate and 1-(6-bromo-3-(1-hydroxyethyl)-1H-indol-1-yl)-2-methylpropan-1-one. ESI-MS (m/z): 517.86, 519.88 [M+1]⁺. ¹HNMR (CDCl₃, 400 MHz) δ 8.72 (d, J=1.6 Hz, 1H), 7.64 (d, J=8.0 Hz, 1H), 7.41 (dd, J=8.0 Hz, 1.6 Hz, 1H), 7.09 (s, 1H), 5.80 (m, 1H), 3.38 (m, 1H), 2.95 (s, 6H), 1.89 (d, J=8.0 Hz, 3H), 1.50 (s, 9H), 1.35 (d, J=7.6 Hz, 6H)

### Step 5: 1-(3-{1-[(dimethylsulfamoyl)amino]ethyl}-6-(pyridin-4-yl)-1H-indol-1-yl)-2-methylpropan-1-one

The title compound was prepared following the same general protocol as described for Step 5, Example 1, using tert-butyl (1-(6-bromo-1-isobutyryl-1H-indol-3-yl)ethyl)(N,N-dimethylsulfamoyl)carbamate. ESI-MS (m/z): 414.92 [M+1]⁺.

### Example 101: tert-butyl 3-(((1-methylethyl)sulfonamido)methyl)-6-(pyridin-4-yl)-1H-indole-1-carboxylate (181)

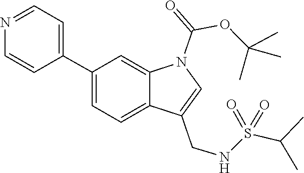 [SMILES: CC(C)S(=O)(=O)NCc1cn(C(=O)OC(C)(C)C)c2cc(-c3ccncc3)ccc12]

### Step 1: tert-butyl 6-bromo-3-formyl-1H-indole-1-carboxylate

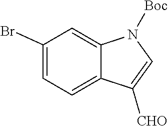 [SMILES: CC(C)(C)OC(=O)n1cc(C=O)c2ccc(Br)cc21]

The title compound was prepared following the same general protocol as described for Step 4, Example 37, using 6-bromo-1H-indole-3-carbaldehyde. ESI-MS (m/z): 323.59, 325.58 [M+1]⁺.

### Step 2: tert-butyl 6-bromo-3-(hydroxymethyl)-1H-indole-1-carboxylate

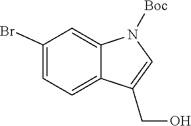 [SMILES: CC(C)(C)OC(=O)n1cc(CO)c2ccc(Br)cc21]

To a solution of tert-butyl 6-bromo-3-formyl-1H-indole-1-carboxylate (1.20 g, 3.71 mmol) in THF (20 mL) at 0° C. was added NaBH₄ (0.17 g, 4.45 mmol) suspended in THF (2 mL). The solution was stirred for 30 min and the completion of the reaction was monitored by reverse phase analytical HPLC. The solution was diluted with EtOAc, followed by washing with sat'd NaHCO₃ and brine and dried over Na₂SO₄ and concentrated in vacuo to obtain the title compound with no further purification. ¹HNMR (CDCl₃, 400 MHz) δ 8.35 (s, 1H), 7.50 (m, 2H), 7.35 (s, 1H), 4.76 (s, 2H), 4.18 (m, 1H), 1.65 (s, 9H)

### Step 3: tert-butyl 6-bromo-3-((1,3-dioxoisoindolin-2-yl)methyl)-1H-indole-1-carboxylate

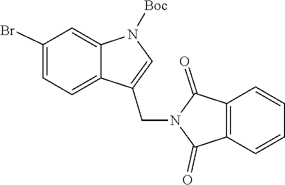 [SMILES: CC(C)(C)OC(=O)n1cc(CN2C(=O)c3ccccc3C2=O)c2ccc(Br)cc21]

The title compound was prepared following the same general protocol as described for Step 6, Example 37, using tert-butyl 6-bromo-3-(hydroxymethyl)-1H-indole-1-carboxylate and isoindoline-1,3-dione. ¹HNMR (CDCl₃, 400 MHz) δ 8.30 (s, 1H), 7.85 (d, J=8.4 Hz, 2H), 7.71 (m, 4H), 7.38 (d, J=1.6 Hz, 1H), 4.92 (s, 2H), 1.65 (s, 9H)

### Step 4: tert-butyl 3-((1,3-dioxoisoindolin-2-yl)methyl)-6-(pyridin-4-yl)-1H-indole-1-carboxylate

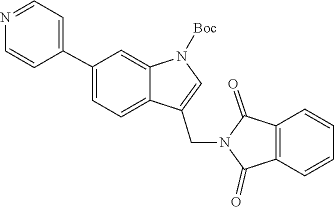 [SMILES: CC(C)(C)OC(=O)n1cc(CN2C(=O)c3ccccc3C2=O)c2ccc(-c3ccncc3)cc21]

The title compound was prepared following the same general protocol as described for Step 5, Example 1, using tert-butyl 6-bromo-3-((1,3-dioxoisoindolin-2-yl)methyl)-1H-indole-1-carboxylate. ESI-MS (m/z): 453.89 [M+1]⁺.

### Step 5: tert-butyl 3-(aminomethyl)-6-(pyridin-4-yl)-1H-indole-1-carboxylate

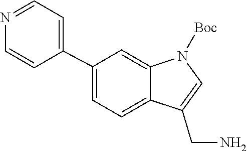 [SMILES: CC(C)(C)OC(=O)n1cc(CN)c2ccc(-c3ccncc3)cc21]

The solution of tert-butyl 3-((1,3-dioxoisoindolin-2-yl)methyl)-6-(pyridin-4-yl)-1H-indole-1-carboxylate (0.039 g, 0.086 mmol) and NH₂NH₂. H₂O (0.009 g, 0.192 mmol) in MeOH (1 mL) was heated in at 70° C. for 2h. The solution was cooled to RT and the solvent was removed in vacuo to obtain the title compound with no further purification. ESI-MS (m/z): 323.79 [M+1]⁺.

### Step 6: tert-butyl 3-(((1-methylethyl)sulfonamido)methyl)-6-(pyridin-4-yl)-1H-indole-1-carboxylate

The title compound was prepared following the same general protocol as described for Step 4, Example 1, using tert-butyl 3-(aminomethyl)-6-(pyridin-4-yl)-1H-indole-1-carboxylate and propane-2-sulfonyl chloride. ESI-MS (m/z): 429.85 [M+1]⁺.

### Example 102: 1-(3-{[(dimethylsulfamoyl)amino]methyl}-6-(pyridin-4-yl)-1H-indol-1-yl)-2-methylpropan-1-one (182)

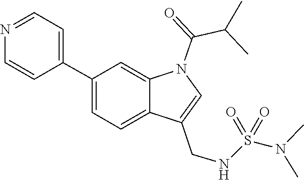 [SMILES: CC(C)C(=O)n1cc(CNS(=O)(=O)N(C)C)c2ccc(-c3ccncc3)cc21]

### Step 1: 1-(6-bromo-3-(hydroxymethyl)-1H-indol-1-yl)-2-methylpropan-1-one

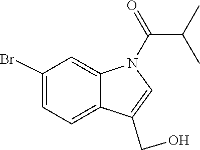 [SMILES: CC(C)C(=O)n1cc(CO)c2ccc(Br)cc21]

The title compound was prepared following the same general protocol as described for Step 2, Example 101, using 6-bromo-1-isobutyryl-1H-indole-3-carbaldehyde. ESI-MS (m/z): 295.72, 297.72 [M+1]⁺.

### Step 2: benzyl ((6-bromo-1-isobutyryl-1H-indol-3-yl)methyl)(N,N-dimethylsulfamoyl)carbamate

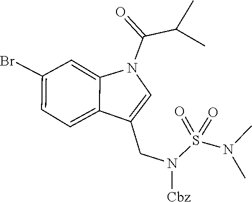 [SMILES: CC(C)C(=O)n1cc(CN(C(=O)OCc2ccccc2)S(=O)(=O)N(C)C)c2ccc(Br)cc21]

The title compound was prepared following the same general protocol as described for Step 6, Example 37, using 1-(6-bromo-3-(hydroxymethyl)-1H-indol-1-yl)-2-methylpropan-1-one. ESI-MS (m/z): 535.78, 537.80 [M+1]⁺. ¹HNMR (CDCl₃, 400 MHz) δ 8.70 (s, 1H), 7.58-7.52 (m, 2H), 7.41-7.38 (m, 6H), 5.38 (s, 2H), 5.03 (s, 2H), 3.12-3.05 (m, 1H), 2.70 (s, 6H), 1.38 (d, J=7.6 Hz, 6H)

### Step 3: 1-(3-{[(dimethylsulfamoyl)amino]methyl}-6-(pyridin-4-yl)-1H-indol-1-yl)-2-methylpropan-1-one

The title compound was prepared following the same general protocol as described for Step 5, Example 1, using benzyl ((6-bromo-1-isobutyryl-1H-indol-3-yl)methyl)(N,N-dimethylsulfamoyl)carbamate. ESI-MS (m/z): 401.05 [M+1]⁺.

### Example 103: 1-(3-{[(dimethylsulfamoyl)amino]methyl}-6-(pyridin-4-yl)-1H-indol-1-yl)-2-methylpropan-1-one (183)

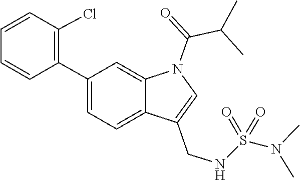 [SMILES: CC(C)C(=O)n1cc(CNS(=O)(=O)N(C)C)c2ccc(-c3ccccc3Cl)cc21]

The title compound was prepared following the same general protocol as described for Step 5, Example 1, using benzyl ((6-bromo-1-isobutyryl-1H-indol-3-yl)methyl)(N,N-dimethylsulfamoyl)carbamate and (2-chlorophenyl)boronic acid. ESI-MS (m/z): 434.96 [M+1]⁺. ¹HNMR (d₆-DMSO, 400 MHz) δ 8.52 (s, 1H), 7.86 (s, 1H), 7.68 (d, J=8.0 Hz, 1H), 7.52 (d, J=8.0 Hz, 1H), 7.44-7.34 (m, 4H), 4.41 (s, 2H), 3.55-3.45 (m, 1H), 2.79 (s, 6H), 1.34 (d, J=7.6 Hz, 6H)

### Example 104: 1-(3-{[(dimethylsulfamoyl)amino]methyl}-6-(1-methyl-1H-pyrazol-5-yl)-1H-indol-1-yl)-2-methylpropan-1-one (184)

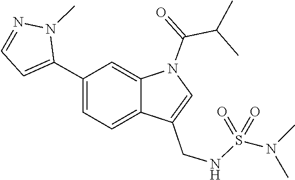 [SMILES: CC(C)C(=O)n1cc(CNS(=O)(=O)N(C)C)c2ccc(-c3ccnn3C)cc21]

The title compound was prepared following the same general protocol as described for Step 5, Example 1, using benzyl ((6-bromo-1-isobutyryl-1H-indol-3-yl)methyl)(N,N-dimethylsulfamoyl)carbamate and 1-methyl-5-(4,4,5,5-tetramethyl-1,3,2-dioxaborolan-2-yl)-1H-pyrazole. ESI-MS (m/z): 403.97 [M+1]⁺.

### Example 105: 1-(3-{[(dimethylsulfamoyl)amino]methyl}-6-[2-(trifluoromethyl)phenyl]-1H-indol-1-yl)-2-methylpropan-1-one (185)

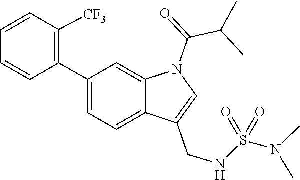 [SMILES: CC(C)C(=O)n1cc(CNS(=O)(=O)N(C)C)c2ccc(-c3ccccc3C(F)(F)F)cc21]

The title compound was prepared following the same general protocol as described for Step 5, Example 1, using benzyl ((6-bromo-1-isobutyryl-1H-indol-3-yl)methyl)(N,N-dimethylsulfamoyl)carbamate and (2-(trifluoromethyl)phenyl)boronic acid. ESI-MS (m/z): 468.49 [M+1]⁺.

### Example 106: 1-(3-{[(dimethylsulfamoyl)amino]methyl}-6-(pyridin-4-yl)-1H-indol-1-yl)-2,2-dimethylpropan-1-one (186)

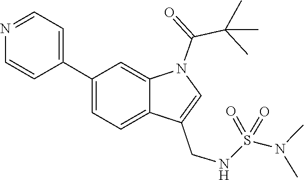 [SMILES: CN(C)S(=O)(=O)NCc1cn(C(=O)C(C)(C)C)c2cc(-c3ccncc3)ccc12]

### Step 1: benzyl 6-bromo-3-formyl-1H-indole-1-carboxylate

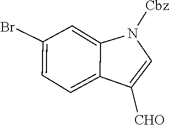 [SMILES: O=Cc1cn(C(=O)OCc2ccccc2)c2cc(Br)ccc12]

The title compound was prepared following the same general protocol as described for Example 34, using 6-bromo-1H-indole-3-carbaldehyde and benzyl chloroformate. ESI-MS (m/z): 357.61, 359.63 [M+1]⁺.

### Step 2: benzyl 6-bromo-3-(hydroxymethyl)-1H-indole-1-carboxylate

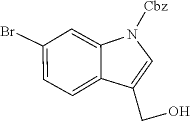 [SMILES: O=C(OCc1ccccc1)n1cc(CO)c2ccc(Br)cc21]

The title compound was prepared following the same general protocol as described for Step 2, Example 101, using benzyl 6-bromo-3-formyl-1H-indole-1-carboxylate. ¹HNMR (CDCl₃, 400 MHz) δ 8.40 (s, 1H), 7.70-7.30 (m, 8H), 5.45 (s, 2H), 4.80 (s, 2H)

### Step 3: (6-(pyridin-4-yl)-1H-indol-3-yl)methanol

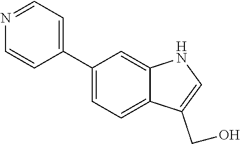 [SMILES: OCc1c[nH]c2cc(-c3ccncc3)ccc12]

The title compound was prepared following the same general protocol as described for Step 5, Example 1, using benzyl 6-bromo-3-(hydroxymethyl)-1H-indole-1-carboxylate. ESI-MS (m/z): 359.03 [M+1]⁺.

### Step 4: tert-butyl (N,N-dimethylsulfamoyl)((6-(pyridin-4-yl)-1H-indol-3-yl)methyl)carbamate

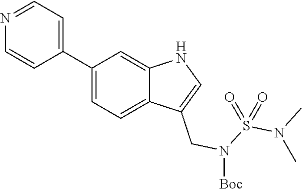 [SMILES: CN(C)S(=O)(=O)N(Cc1c[nH]c2cc(-c3ccncc3)ccc12)C(=O)OC(C)(C)C]

The title compound was prepared following the same general protocol as described for Step 7, Example 37, using tert-butyl (N,N-dimethylsulfamoyl)carbamate and (6-(pyridin-4-yl)-1H-indol-3-yl)methanol. ESI-MS (m/z): 430.88 [M+1]⁺.

### Step 5: tert-butyl (N,N-dimethylsulfamoyl)((1-pivaloyl-6-(pyridin-4-yl)-1H-indol-3-yl)methyl)carbamate

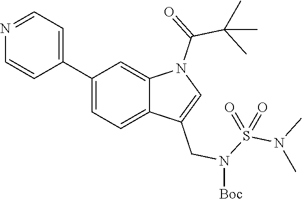 [SMILES: CN(C)S(=O)(=O)N(Cc1cn(C(=O)C(C)(C)C)c2cc(-c3ccncc3)ccc12)C(=O)OC(C)(C)C]

The title compound was prepared following the same general protocol as described for Step 4, Example 37, using tert-butyl (N,N-dimethylsulfamoyl)((6-(pyridin-4-yl)-1H-indol-3-yl)methyl)carbamate and pivaloyl chloride. ESI-MS (m/z): 514.91 [M+1]⁺.

### Step 6: 1-(3-{[(dimethylsulfamoyl)amino]methyl}-6-(pyridin-4-yl)-1H-indol-1-yl)-2,2-dimethylpropan-1-one

The title compound was prepared following the same general protocol as described for Example 25, using tert-butyl (N,N-dimethylsulfamoyl)((1-pivaloyl-6-(pyridin-4-yl)-1H-indol-3-yl)methyl)carbamate. ESI-MS (m/z): 415.93 [M+1]⁺.

### Example 107: 1-(3-{[(dimethylsulfamoyl)amino]methyl}-6-(pyridin-4-yl)-1H-indol-1-yl)propan-1-one (187)

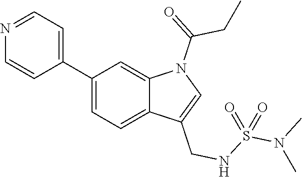 [SMILES: CCC(=O)n1cc(CNS(=O)(=O)N(C)C)c2ccc(-c3ccncc3)cc21]

### Step 1: tert-butyl (N,N-dimethylsulfamoyl)((1-propionyl-6-(pyridin-4-yl)-1H-indol-3-yl)methyl)carbamate

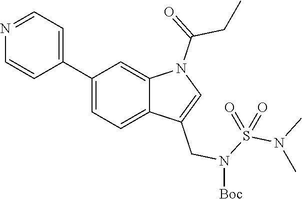 [SMILES: CCC(=O)n1cc(CN(C(=O)OC(C)(C)C)S(=O)(=O)N(C)C)c2ccc(-c3ccncc3)cc21]

The title compound was prepared following the same general protocol as described for Step 4, Example 37, using tert-butyl (N,N-dimethylsulfamoyl)((6-(pyridin-4-yl)-1H-indol-3-yl)methyl)carbamate and propionyl chloride. ESI-MS (m/z): 486.88 [M+1]⁺.

### Step 2: 1-(3-{[(dimethylsulfamoyl)amino]methyl}-6-(pyridin-4-yl)-1H-indol-1-yl)propan-1-one

The title compound was prepared following the same general protocol as described for Example 25, using tert-butyl (N,N-dimethylsulfamoyl)((1-propionyl-6-(pyridin-4-yl)-1H-indol-3-yl)methyl)carbamate. ESI-MS (m/z): 387.16 [M+1]⁺.

### Example 108: 1-(3-{[(dimethylsulfamoyl)amino]methyl}-6-(pyridin-4-yl)-1H-indol-1-yl)-2-methylbutan-1-one (188)

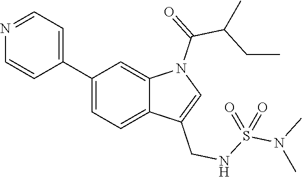 [SMILES: CCC(C)C(=O)n1cc(CNS(=O)(=O)N(C)C)c2ccc(-c3ccncc3)cc21]

### Step 1: tert-butyl (N,N-dimethylsulfamoyl)((1-(2-methylbutanoyl)-6-(pyridin-4-yl)-1H-indol-3-yl)methyl)carbamate

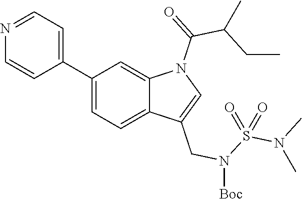 [SMILES: CCC(C)C(=O)n1cc(CN(C(=O)OC(C)(C)C)S(=O)(=O)N(C)C)c2ccc(-c3ccncc3)cc21]

The title compound was prepared following the same general protocol as described for Step 4, Example 37, using tert-butyl (N,N-dimethylsulfamoyl)((6-(pyridin-4-yl)-1H-indol-3-yl)methyl)carbamate and propionyl chloride. ESI-MS (m/z): 514.99 [M+1]⁺.

### Step 2: 1-(3-{[(dimethylsulfamoyl)amino]methyl}-6-(pyridin-4-yl)-1H-indol-1-yl)-2-methylbutan-1-one

The title compound was prepared following the same general protocol as described for Example 25, using tert-butyl (N,N-dimethylsulfamoyl)((1-(2-methylbutanoyl)-6-(pyridin-4-yl)-1H-indol-3-yl)methyl)carbamate. ESI-MS (m/z): 415.04 [M+1]⁺.

### Example 109: 1-(3-{1-[(dimethylsulfamoyl)amino]ethyl}-6-[2-(trifluoromethyl)phenyl]-1H-indol-1-yl)-2-methylpropan-1-one (195)

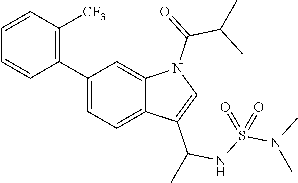 [SMILES: CC(C)C(=O)n1cc(C(C)NS(=O)(=O)N(C)C)c2ccc(-c3ccccc3C(F)(F)F)cc21]

### Step 1: 6-(2-(trifluoromethyl)phenyl)-1H-indole-3-carbaldehyde

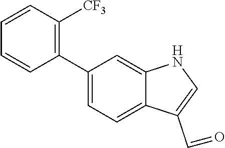 [SMILES: O=Cc1c[nH]c2cc(-c3ccccc3C(F)(F)F)ccc12]

The title compound was prepared following the same general protocol as described for Step 5, Example 1, using benzyl 6-bromo-3-formyl-1H-indole-1-carboxylate and (2-(trifluoromethyl)phenyl)boronic acid. ESI-MS (m/z): 289.87 [M+1]⁺.

### Step 2: 1-isobutyryl-6-(2-(trifluoromethyl)phenyl)-1H-indole-3-carbaldehyde

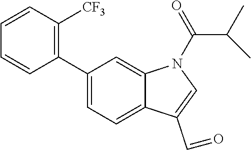 [SMILES: CC(C)C(=O)n1cc(C=O)c2ccc(-c3ccccc3C(F)(F)F)cc21]

The title compound was prepared following the same general protocol as described for Step 4, Example 37, using 6-(2-(trifluoromethyl)phenyl)-1H-indole-3-carbaldehyde and isobutyryl chloride. ESI-MS (m/z): 359.78 [M+1]⁺.

### Step 3: 1-(3-(1-hydroxyethyl)-6-(2-(trifluoromethyl)phenyl)-1H-indol-1-yl)-2-methylpropan-1-one

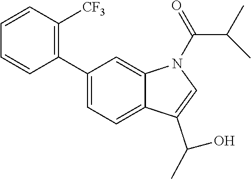 [SMILES: CC(C)C(=O)n1cc(C(C)O)c2ccc(-c3ccccc3C(F)(F)F)cc21]

The title compound was prepared following the same general protocol as described for Step 3, Example 100, using 1-isobutyryl-6-(2-(trifluoromethyl)phenyl)-1H-indole-3-carbaldehyde. ESI-MS (m/z): 375.79 [M+1]⁺.

### Step 4: benzyl (N,N-dimethylsulfamoyl)(1-(1-isobutyryl-6-(2-(trifluoromethyl)phenyl)-1H-indol-3-yl)ethyl)carbamate

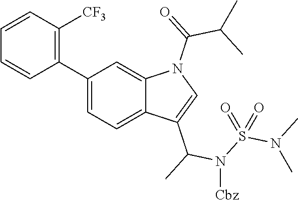 [SMILES: CC(C)C(=O)n1cc(C(C)N(C(=O)OCc2ccccc2)S(=O)(=O)N(C)C)c2ccc(-c3ccccc3C(F)(F)F)cc21]

The title compound was prepared following the same general protocol as described for Step 7, Example 37, using 1-(3-(1-hydroxyethyl)-6-(2-(trifluoromethyl)phenyl)-1H-indol-1-yl)-2-methylpropan-1-one. ESI-MS (m/z): 616.08 [M+1]⁺.

### Step 5: 1-(3-{1-[(dimethylsulfamoyl)amino]ethyl}-6-[2-(trifluoromethyl)phenyl]-1H-indol-1-yl)-2-methylpropan-1-one

The title compound was prepared following the same general protocol as described for Example 20, using benzyl (N,N-dimethylsulfamoyl)(1-(1-isobutyryl-6-(2-(trifluoromethyl)phenyl)-1H-indol-3-yl)ethyl)carbamate. ESI-MS (m/z): 481.45 [M+1]⁺.

### Example 111: 1-[6-(2-chlorophenyl)-3-{1-[(dimethylsulfamoyl)amino]ethyl}-1H-indol-1-yl]-2-methylpropan-1-one (196)

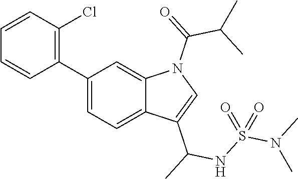 [SMILES: CC(C)C(=O)n1cc(C(C)NS(=O)(=O)N(C)C)c2ccc(-c3ccccc3Cl)cc21]

### Step 1: 6-(2-chlorophenyl)-1H-indole-3-carbaldehyde

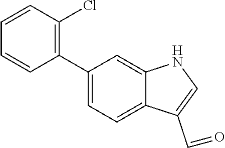 [SMILES: O=Cc1c[nH]c2cc(-c3ccccc3Cl)ccc12]

The title compound was prepared following the same general protocol as described for Step 5, Example 1, using benzyl 6-bromo-3-formyl-1H-indole-1-carboxylate and (2-chlorophenyl)boronic acid. ESI-MS (m/z): 255.76 [M+1]⁺.

### Step 2: 6-(2-chlorophenyl)-1-isobutyryl-1H-indole-3-carbaldehyde

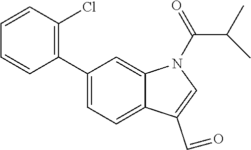 [SMILES: CC(C)C(=O)n1cc(C=O)c2ccc(-c3ccccc3Cl)cc21]

The title compound was prepared following the same general protocol as described for Step 4, Example 37, using 6-(2-chlorophenyl)-1H-indole-3-carbaldehyde and isobutyryl chloride. ESI-MS (m/z): 325.72 [M+1]⁺.

### Step 3: 1-(6-(2-chlorophenyl)-3-(1-hydroxyethyl)-1H-indol-1-yl)-2-methylpropan-1-one

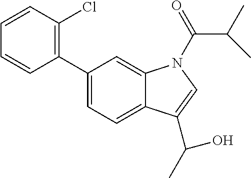 [SMILES: CC(C)C(=O)n1cc(C(C)O)c2ccc(-c3ccccc3Cl)cc21]

The title compound was prepared following the same general protocol as described for Step 3, Example 100, using 6-(2-chlorophenyl)-1-isobutyryl-1H-indole-3-carbaldehyde. ESI-MS (m/z): 341.72 [M+1]⁺.

### Step 4: benzyl (1-(6-(2-chlorophenyl)-1-isobutyryl-1H-indol-3-yl)ethyl)(N,N-dimethylsulfamoyl)carbamate

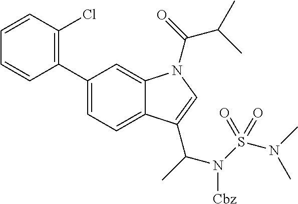 [SMILES: CC(C)C(=O)n1cc(C(C)N(C(=O)OCc2ccccc2)S(=O)(=O)N(C)C)c2ccc(-c3ccccc3Cl)cc21]

The title compound was prepared following the same general protocol as described for Step 7, Example 37, using 1-(6-(2-chlorophenyl)-3-(1-hydroxyethyl)-1H-indol-1-yl)-2-methylpropan-1-one. ESI-MS (m/z): 582.65 [M+1]⁺.

### Step 5: 1-[6-(2-chlorophenyl)-3-{1-[(dimethylsulfamoyl)amino]ethyl}-1H-indol-1-yl]-2-methylpropan-1-one

The title compound was prepared following the same general protocol as described for Example 20, using benzyl (1-(6-(2-chlorophenyl)-1-isobutyryl-1H-indol-3-yl)ethyl)(N,N-dimethylsulfamoyl)carbamate. ESI-MS (m/z): 447.57 [M+1]⁺.

### Example 112: 1-(3-{1-[(dimethylsulfamoyl)amino]ethyl}-6-(pyridin-4-yl)-1H-indol-1-yl)-2,2-dimethylpropan-1-one (197)

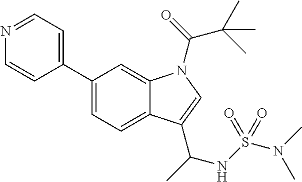 [SMILES: CC(NS(=O)(=O)N(C)C)c1cn(C(=O)C(C)(C)C)c2cc(-c3ccncc3)ccc12]

### Step 1: 6-(pyridin-4-yl)-1H-indole-3-carbaldehyde

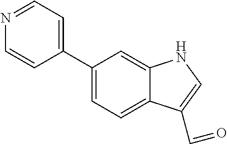 [SMILES: O=Cc1c[nH]c2cc(-c3ccncc3)ccc12]

The title compound was prepared following the same general protocol as described for Step 5, Example 1, using benzyl 6-bromo-3-formyl-1H-indole-1-carboxylate. ESI-MS (m/z): 222.87 [M+1]⁺.

### Step 2: 6-(2-chlorophenyl)-1-isobutyryl-1H-indole-3-carbaldehyde

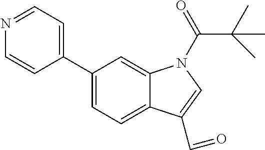 [SMILES: CC(C)(C)C(=O)n1cc(C=O)c2ccc(-c3ccncc3)cc21]

The title compound was prepared following the same general protocol as described for Example 34, using 6-(pyridin-4-yl)-1H-indole-3-carbaldehyde and pivaloyl chloride. ESI-MS (m/z): 306.79 [M+1]⁺.

### Step 3: 1-(6-(2-chlorophenyl)-3-(1-hydroxyethyl)-1H-indol-1-yl)-2-methylpropan-1-one

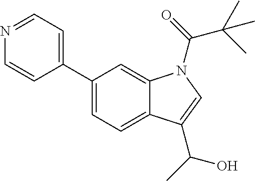 [SMILES: CC(O)c1cn(C(=O)C(C)(C)C)c2cc(-c3ccncc3)ccc12]

The title compound was prepared following the same general protocol as described for Step 3, Example 100, using 1-(3-(1-hydroxyethyl)-6-(pyridin-4-yl)-1H-indol-1-yl)-2,2-dimethylpropan-1-one. ESI-MS (m/z): 322.97 [M+1]⁺.

### Step 4: benzyl (N,N-dimethylsulfamoyl)(1-(1-pivaloyl-6-(pyridin-4-yl)-1H-indol-3-yl)ethyl)carbamate

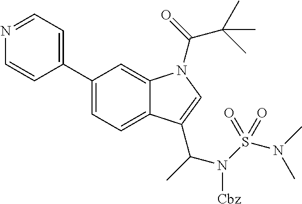 [SMILES: CC(c1cn(C(=O)C(C)(C)C)c2cc(-c3ccncc3)ccc12)N(C(=O)OCc1ccccc1)S(=O)(=O)N(C)C]

The title compound was prepared following the same general protocol as described for Step 7, Example 37, using 1-(3-(1-hydroxyethyl)-6-(pyridin-4-yl)-1H-indol-1-yl)-2,2-dimethylpropan-1-one. ESI-MS (m/z): 563.03 [M+1]⁺.

### Step 5: 1-(3-{1-[(dimethylsulfamoyl)amino]ethyl}-6-(pyridin-4-yl)-1H-indol-1-yl)-2,2-dimethylpropan-1-one

The title compound was prepared following the same general protocol as described for Example 20, using benzyl (N,N-dimethylsulfamoyl)(1-(1-pivaloyl-6-(pyridin-4-yl)-1H-indol-3-yl)ethyl)carbamate. ESI-MS (m/z): 428.99 [M+1]⁺.

### Example 113: N-(1-(1-isobutyl-6-(2-(trifluoromethyl)phenyl)-1H-indol-3-yl)ethyl)cyclopropanesulfonamide (204)

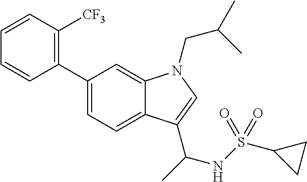 [SMILES: CC(C)Cn1cc(C(C)NS(=O)(=O)C2CC2)c2ccc(-c3ccccc3C(F)(F)F)cc21]

### Step 1: 1-(6-bromo-1H-indol-3-yl)ethan-1-one

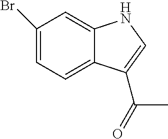

The title compound was prepared following the same general protocol as described for Step 1, Example 87, using 6-bromoindole and acetyl chloride. ESI-MS (m/z): 237.79, 239.77 [M+1]⁺.

### Step 2: 1-(6-bromo-1-isobutyl-1H-indol-3-yl)ethan-1-one

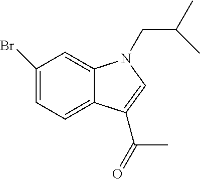 [SMILES: CC(=O)c1cn(CC(C)C)c2cc(Br)ccc12]

The title compound was prepared following the same general protocol as described for Step 1, Example 1, using 1-(6-bromo-1H-indol-3-yl)ethan-1-one. ESI-MS (m/z): 293.89, 295.89 [M+1]⁺.

### Step 3: 1-(1-isobutyl-6-(2-(trifluoromethyl)phenyl)-1H-indol-3-yl)ethan-1-one

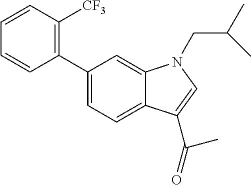 [SMILES: CC(=O)c1cn(CC(C)C)c2cc(-c3ccccc3C(F)(F)F)ccc12]

The title compound was prepared following the same general protocol as described for Step 5, Example 1, using 1-(6-bromo-1-isobutyl-1H-indol-3-yl)ethan-1-one and (2-(trifluoromethyl)phenyl)boronic acid. ESI-MS (m/z): 359.78 [M+1]⁺.

### Step 4: (Z)—N-(1-(1-isobutyl-6-(2-(trifluoromethyl)phenyl)-1H-indol-3-yl)ethylidene)cyclopropanesulfonamide

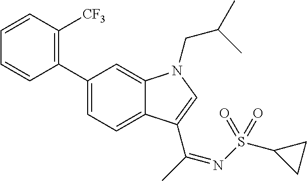 [SMILES: C/C(=N/S(=O)(=O)C1CC1)c1cn(CC(C)C)c2cc(-c3ccccc3C(F)(F)F)ccc12]

A solution of 1-(1-isobutyl-6-(2-(trifluoromethyl)phenyl)-1H-indol-3-yl)ethan-1-one (0.1 g, 0.278 mmol and cyclopropanesulfonamide in Ti(OEt)₄ (1 mL) was heated at 130° C. overnight. The solution was cooled to RT and diluted with EtOAc and brine. The mixture was filtered and the filtrate was concentrated in vacuo to obtain the crude which was purified by chromatography on silica gel (EtOAc/hex) to afford the title compound. ESI-MS (m/z): 463.06 [M+1]⁺.

### Step 5: N-(1-(1-isobutyl-6-(2-(trifluoromethyl)phenyl)-1H-indol-3-yl)ethyl)cyclopropanesulfonamide

The title compound was prepared following the same general protocol as described for Step 3, Example 1, using (Z)—N-(1-(1-isobutyl-6-(2-(trifluoromethyl)phenyl)-1H-indol-3-yl)ethylidene)cyclopropanesulfonamide. ESI-MS (m/z): 465.20 [M+1]⁺.

### Example 114: N-(2-(1-isobutyl-6-(2-(trifluoromethyl)phenyl)-1H-indol-3-yl)propan-2-yl)cyclopropanesulfonamide (205)

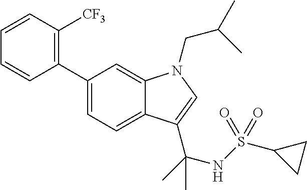 [SMILES: CC(C)Cn1cc(C(C)(C)NS(=O)(=O)C2CC2)c2ccc(-c3ccccc3C(F)(F)F)cc21]

The title compound was prepared following the same general protocol as described for Step 3, Example 100, using (Z)—N-(1-(1-isobutyl-6-(2-(trifluoromethyl)phenyl)-1H-indol-3-yl)ethylidene)cyclopropanesulfonamide. ESI-MS (m/z): 477.94 [M+1]⁺.

### Example 115: dimethyl({1-[1-(2-methylpropyl)-6-(pyridin-4-yl)-1H-indol-3-yl]cyclopropyl}sulfamoyl)amine (207)

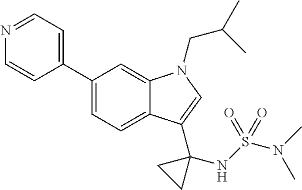 [SMILES: CC(C)Cn1cc(C2(NS(=O)(=O)N(C)C)CC2)c2ccc(-c3ccncc3)cc21]

### Step 1: 1-(6-bromo-1H-indol-3-yl)-2,2,2-trifluoroethan-1-one

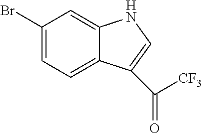 [SMILES: O=C(c1c[nH]c2cc(Br)ccc12)C(F)(F)F]

To a solution of 6-bromoindole (5.116 g, 26.09 mmol) in DMF at 0° C. was added TFAA (4.3 mL, 31.308 mmol). The solution was stirred overnight. The reaction was poured into ice water and then filtered to obtain the title compound as solid with no purification. ESI-MS (m/z): 293.03, 295.55 [M+1]⁺.

### Step 2: 6-bromo-1H-indole-3-carboxylic acid

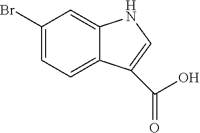 [SMILES: O=C(O)c1c[nH]c2cc(Br)ccc12]

To a solution of 1-(6-bromo-1H-indol-3-yl)-2,2,2-trifluoroethan-1-one (2.5 g, 8.6 mmol) in water (30 mL) was added NaOH (3.44 g, 86 mmol). The reaction mixture was heated in a 100° C. oil bath overnight. The solution was cooled to RT and acidified with con. HCl and concentrated in vacuo. The crude was slurried in MeOH and the salts were filtered off. The filtrate was used for next Step with no purification. ESI-MS (m/z): 240.91, 241.86 [M+1]⁺.

### Step 3: methyl 6-bromo-1H-indole-3-carboxylate

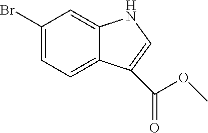

To a solution of the crude from the previous step in MeOH was added a few drops of H₂SO₄. The solution was refluxed for 18h, and then cooled to RT and concentrated in vacuo. The crude residue was re-dissolved in EtOAc, washed with water, sat'd NaHCO₃, brine, dried (Na₂SO₄) and filtered. The solvent was removed in vacuo to obtain the title compound with no further purification. ESI-MS (m/z): 253.73, 255.75 [M+1]⁺.

### Step 4: methyl 6-(pyridin-4-yl)-1H-indole-3-carboxylate

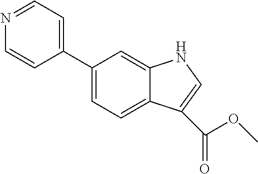 [SMILES: COC(=O)c1c[nH]c2cc(-c3ccncc3)ccc12]

The title compound was prepared following the same general protocol as described for Step 5, Example 1, using methyl 6-bromo-1H-indole-3-carboxylate. ESI-MS (m/z): 252.86 [M+1]⁺.

### Step 5: methyl 1-isobutyl-6-(pyridin-4-yl)-1H-indole-3-carboxylate

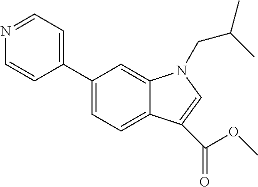 [SMILES: COC(=O)c1cn(CC(C)C)c2cc(-c3ccncc3)ccc12]

The title compound was prepared following the same general protocol as described for Step 1, Example 1, using methyl 6-(pyridin-4-yl)-1H-indole-3-carboxylate. ESI-MS (m/z): 309.03 [M+1]⁺.

### Step 6: 1-(1-isobutyl-6-(pyridin-4-yl)-1H-indol-3-yl)cyclopropan-1-ol

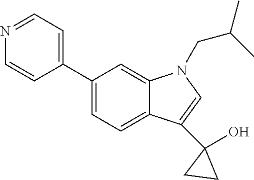 [SMILES: CC(C)Cn1cc(C2(O)CC2)c2ccc(-c3ccncc3)cc21]

To a solution of methyl 1-isobutyl-6-(pyridin-4-yl)-1H-indole-3-carboxylate (0.231 g, 0.749 mmol) and Ti(Oi-Pr)₄ (0.234 g, 8.239 mmol) in THF (4 mL) at 0° C. was added dropwise EtMgBr (1.3 mL, 3M in ether, 3.90 mmol). The solution was allowed to warm to RT and stirred overnight. The reaction was quenched by addition of water and diluted with EtOAc. The layers were separated, and the aqueous phase was extracted with EtOAc. The combined organics were concentrated in vacuo to obtain the crude which was purified by chromatography on silica gel (EtOAc/hex) to afford the title compound. ESI-MS (m/z): 307.08 [M+1]⁺.

### Step 7: benzyl (N,N-dimethylsulfamoyl)(1-(1-isobutyl-6-(pyridin-4-yl)-1H-indol-3-yl)cyclopropyl)carbamate

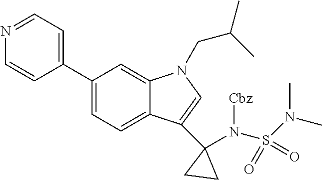 [SMILES: CC(C)Cn1cc(C2(N(C(=O)OCc3ccccc3)S(=O)(=O)N(C)C)CC2)c2ccc(-c3ccncc3)cc21]

The title compound was prepared following the same general protocol as described for Step 7, Example 37, using 1-(1-isobutyl-6-(pyridin-4-yl)-1H-indol-3-yl)cyclopropan-1-ol. ESI-MS (m/z): 547.15 [M+1]⁺.

### Step 8: dimethyl({1-[1-(2-methylpropyl)-6-(pyridin-4-yl)-1H-indol-3-yl]cyclopropyl}sulfamoyl)amine

The title compound was prepared following the same general protocol as described for Example 20, using benzyl (N,N-dimethylsulfamoyl)(1-(1-isobutyl-6-(pyridin-4-yl)-1H-indol-3-yl)cyclopropyl)carbamate. ESI-MS (m/z): 413.95 [M+1]⁺.

### Example 116: dimethyl({1-[1-(2-methylpropyl)-6-(pyridin-4-yl)-1H-indol-3-yl]cyclopropyl}sulfamoyl)amine (208)

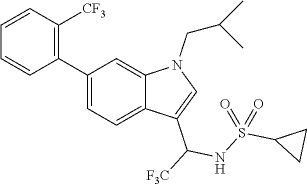 [SMILES: CC(C)Cn1cc(C(NS(=O)(=O)C2CC2)C(F)(F)F)c2ccc(-c3ccccc3C(F)(F)F)cc21]

### Step 1: 1-isobutyl-6-(2-(trifluoromethyl)phenyl)-1H-indole

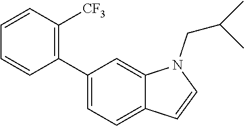 [SMILES: CC(C)Cn1ccc2ccc(-c3ccccc3C(F)(F)F)cc21]

The title compound was prepared following the same general protocol as described for Step 5, Example 1, using 6-bromo-1-isobutyl-1H-indole and (2-(trifluoromethyl)phenyl)boronic acid. ESI-MS (m/z): 317.86 [M+1]⁺.

### Step 2: 2-methyl-N-(3,3,3-trifluoroprop-1-en-1-yl)propane-2-sulfinamide

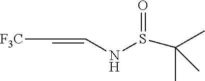

To a solution of 2,2,2-trifluoroethane-1,1-diol (2.93 g, 75% in water, 18.906 mmol) and 2-methylpropane-2-sulfinamide (2.98 g, 24.578 mmol) in anhydrous DCM (38 mL) was added anhydrous MgSO₄ (5.6 g, 37.812 mmol) and 4A MS (19 g). The mixture was heated in a 40° C. oil bath overnight. The solution was cooled to RT, filtered, and used in the next without further purification.

### Step 3: 2-methyl-N-(2,2,2-trifluoro-1-(1-isobutyl-6-(2-(trifluoromethyl)phenyl)-1H-indol-3-yl)ethyl)propane-2-sulfinamide

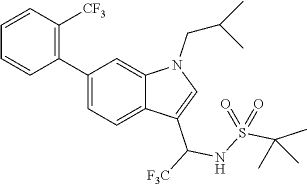 [SMILES: CC(C)Cn1cc(C(NS(=O)(=O)C(C)(C)C)C(F)(F)F)c2ccc(-c3ccccc3C(F)(F)F)cc21]

To a solution of 1-isobutyl-6-(2-(trifluoromethyl)phenyl)-1H-indole (6.0 g, 18.906 mmol) in DCM (24 mL) cooled to −10° C. was added BF₃·OEt₂ (3.05 mL) followed by addition of the imine solution obtained from the previous step. The solution was maintained at −10° C. for 1-2 h monitoring completion of the reaction as judged by TLC (consumption of indole starting material). The solution was diluted with DCM, followed by water. The aqueous layer was extracted with DCM twice. The combined organics were washed with brine, dried (MgSO₄) and concentrated in vacuo in a 25° C. water bath to obtain the crude which was purified by chromatography on silica gel (EtOAc/hex) to afford the title compound. ESI-MS (m/z): 518.70 [M+1]⁺. ¹HNMR (CDCl₃, 400 MHz) δ 7.86 (d, J=8.2 Hz, 1H), 7.74 (d, J=8.0 Hz, 1H), 7.67 (t, J=8.0 Hz, 1H), 7.58 (t, J=8.0 Hz, 1H), 7.46 (m, 3H), 7.10 (d, J=8.0 Hz, 1H), 5.05 (m, 1H), 4.12 (m, 1H), 3.98 (m, 2H), 2.18 (m, 1H), 1.3 (m, 9H), 0.97 (m, 6H)

### Step 4: 2,2,2-trifluoro-1-(1-isobutyl-6-(2-(trifluoromethyl)phenyl)-1H-indol-3-yl)ethan-1-amine

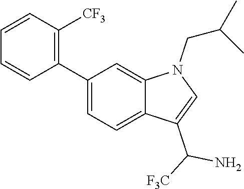 [SMILES: CC(C)Cn1cc(C(N)C(F)(F)F)c2ccc(-c3ccccc3C(F)(F)F)cc21]

To 2-methyl-N-(2,2,2-trifluoro-1-(1-isobutyl-6-(2-(trifluoromethyl)phenyl)-1H-indol-3-yl)ethyl)propane-2-sulfinamide (4.46 g, 8.6 mmol) in MeOH (80 mL, 0.1M) at RT was slowly added con. HCl (2.6 mL, 36%, 0.5 mL/mmol). The solution was stirred for 1-2 h and the completion of reaction was monitored by reverse phase analytical HPLC. The solution was concentrated in vacuo in a 25° C. water bath to obtain the crude which was diluted with DCM, followed by addition of TEA (12 mL, 10 eq). The solution was stirred for 20 min and water was added. The layers were separated, and the aqueous layer was extracted with DCM (2×). The combined organics were washed with brine and concentrated in vacuo in a 25° C. water bath to obtain the crude which was purified by chromatography on silica gel (EtOAc/hex) to afford the title compound. ¹HNMR (CD₃CN, 400 MHz) δ 7.83 (d, J=8.2 Hz, 1H), 7.73 (d, J=8.0 Hz, 1H), 7.67 (t, J=8.0 Hz, 1H), 7.56 (t, J=8.0 Hz, 1H), 7.46 (m, 3H), 7.10 (d, J=8.0 Hz, 1H), 5.05 (m, 1H), 3.98 (d, J=8.0 Hz, 2H), 2.18 (m, 1H), 0.97 (d, J=7.6 Hz, 6H)

### Step 5: dimethyl({1-[1-(2-methylpropyl)-6-(pyridin-4-yl)-1H-indol-3-yl]cyclopropyl}sulfamoyl)amine

To a solution of 2,2,2-trifluoro-1-(1-isobutyl-6-(2-(trifluoromethyl)phenyl)-1H-indol-3-yl)ethan-1-amine (0.1 g, 0.24 mmol, 1.0 eq) in anhydrous pyridine (0.3 mL) at RT was added cyclopropanesulfonyl chloride (0.067 g, 2.0 eq). The solution was stirred at RT overnight and the completion of reaction was monitored by reverse phase analytical HPLC. The solution was concentrated in vacuo in a 25° C. water bath to obtain the crude which was diluted with EtOAc, washed with 2N HCl, water, sat'd NaHCO₃, brine, dried (Na₂SO₄) and concentrated. The solvent was removed in vacuo in a 25° C. water bath to obtain the crude which was purified by chromatography on silica gel (EtOAc/hex) to afford the title compound. ESI-MS (m/z): 518.76 [M+1]⁺. ¹HNMR (CDCl₃, 400 MHz) δ 7.77 (t, J=8.0 Hz, 2H), 7.57 (d, J=8.0 Hz, 1H), 7.45 (d, J=8.0 Hz, 1H), 7.28 (s, 1H), 7.13-7.05 (m, 2H), 5.04-4.98 (m, 1H), 4.61 (d, J=4.0 Hz, 1H), 3.87 (d, J=8.0 Hz, 2H), 2.27-2.24 (m, 1H), 2.18-2.13 (m, 1H), 1.18-1.03 (m, 2H), 0.98-0.85 (m, 8H)

### Example 117: dimethyl({2,2,2-trifluoro-1-[1-(2-methylpropyl)-6-[2-(trifluoromethyl)phenyl]-1H-indol-3-yl]ethyl}sulfamoyl)amine (223)

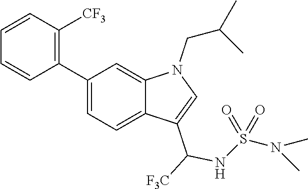 [SMILES: CC(C)Cn1cc(C(NS(=O)(=O)N(C)C)C(F)(F)F)c2ccc(-c3ccccc3C(F)(F)F)cc21]

The title compound was prepared following the same general protocol as described for Step 5, Example 116, using dimethylsulfamoyl chloride. ESI-MS (m/z): 521.90 [M+1]⁺. ¹HNMR (CDCl₃, 400 MHz) δ 7.78-7.70 (m, 2H), 7.57 (t, J=8.0 Hz, 1H), 7.48 (t, J=8.0 Hz, 1H), 7.41 (d, J=4.0 Hz, 1H), 7.31 (s, 1H), 7.25 (d, J=8.0 Hz, 1H), 7.15 (d, J=8.0 Hz, 1H), 5.26 (q, J=8.0 Hz, 1H), 4.78 (d, J=8.0 Hz, 1H), 3.90 (d, J=8.0 Hz, 2H), 2.76 (s, 6H), 2.26-2.08 (m, 1H), 0.92 (d, J=8.0 Hz, 6H)

### Example 118: N-(2,2,2-trifluoro-1-(1-isobutyl-6-(2-(trifluoromethyl)phenyl)-1H-indol-3-yl)ethyl)propane-2-sulfonamide (224)

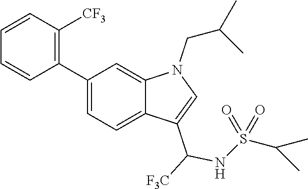 [SMILES: CC(C)Cn1cc(C(NS(=O)(=O)C(C)C)C(F)(F)F)c2ccc(-c3ccccc3C(F)(F)F)cc21]

The title compound was prepared following the same general protocol as described for Step 5, Example 116, using propane-2-sulfonyl chloride. ESI-MS (m/z): 521.83 [M+1]⁺.

### Example 119: N-(2,2,2-trifluoro-1-(1-isobutyl-6-(2-(trifluoromethyl)phenyl)-1H-indol-3-yl)ethyl)propane-2-sulfonamide (221)

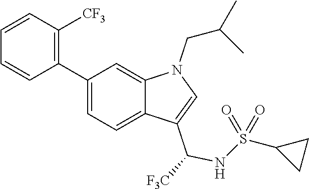 [SMILES: CC(C)Cn1cc([C@H](NS(=O)(=O)C2CC2)C(F)(F)F)c2ccc(-c3ccccc3C(F)(F)F)cc21]

### Step 1: (S)-2-methyl-N-(3,3,3-trifluoroprop-1-en-1-yl)propane-2-sulfinamide

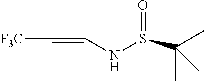

The title compound was prepared following the same general protocol as described for Step 2, Example 116, using (S)-2-methylpropane-2-sulfinamide.

### Step 2: (S)-2-methyl-N—((S)-2,2,2-trifluoro-1-(1-isobutyl-6-(2-(trifluoromethyl)phenyl)-1H-indol-3-yl)ethyl)propane-2-sulfinamide

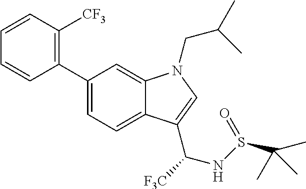 [SMILES: CC(C)Cn1cc([C@H](N[S@@](=O)C(C)(C)C)C(F)(F)F)c2ccc(-c3ccccc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide. ESI-MS (m/z): 518.60 [M+1]⁺. ESI-MS (m/z): 518.70 [M+1]⁺. ¹HNMR (CDCl₃, 400 MHz) δ 7.76 (d, J=8.2 Hz, 1H), 7.65 (d, J=8.0 Hz, 1H), 7.56 (t, J=8.0 Hz, 1H), 7.58-7.41 (m, 2H), 7.30 (s, 1H), 7.26 (s, 1H), 7.10 (d, J=8.0 Hz, 1H), 5.22-5.13 (m, 1H), 3.98-3.95 (m, 1H), 3.93-3.82 (m, 2H), 2.22-2.14 (m, 1H), 1.22 (s, 9H), 0.92 (m, 6H)

### Step 3: (S)-2,2,2-trifluoro-1-(1-isobutyl-6-(2-(trifluoromethyl)phenyl)-1H-indol-3-yl)ethan-1-amine

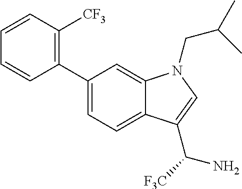 [SMILES: CC(C)Cn1cc([C@H](N)C(F)(F)F)c2ccc(-c3ccccc3C(F)(F)F)cc21]

The title compound was prepared following the same general protocol as described for Step 4, Example 116, using (S)-2-methyl-N—((S)-2,2,2-trifluoro-1-(1-isobutyl-6-(2-(trifluoromethyl)phenyl)-1H-indol-3-yl)ethyl)propane-2-sulfinamide.

### Step 4: N-(2,2,2-trifluoro-1-(1-isobutyl-6-(2-(trifluoromethyl)phenyl)-1H-indol-3-yl)ethyl)propane-2-sulfonamide

The title compound was prepared following the same general protocol as described for Step 5, Example 116, using (S)-2,2,2-trifluoro-1-(1-isobutyl-6-(2-(trifluoromethyl)phenyl)-1H-indol-3-yl)ethan-1-amine. ESI-MS (m/z): 518.73 [M+1]⁺.

### Example 120: (R)—N-(2,2,2-trifluoro-1-(1-isobutyl-6-(2-(trifluoromethyl)phenyl)-1H-indol-3-yl)ethyl)cyclopropanesulfonamide (222)

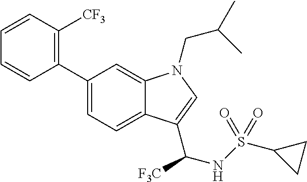 [SMILES: CC(C)Cn1cc([C@@H](NS(=O)(=O)C2CC2)C(F)(F)F)c2ccc(-c3ccccc3C(F)(F)F)cc21]

### Step 1: (R)-2-methyl-N-(3,3,3-trifluoroprop-1-en-1-yl)propane-2-sulfinamide

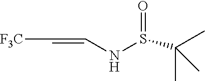

The title compound was prepared following the same general protocol as described for Step 2, Example 116, using (R)-2-methylpropane-2-sulfinamide.

### Step 2: (R)-2-methyl-N—((R)-2,2,2-trifluoro-1-(1-isobutyl-6-(2-(trifluoromethyl)phenyl)-1H-indol-3-yl)ethyl)propane-2-sulfinamide

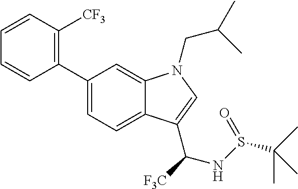 [SMILES: CC(C)Cn1cc([C@@H](N[S@](=O)C(C)(C)C)C(F)(F)F)c2ccc(-c3ccccc3C(F)(F)F)cc21]

The title compound was prepared following the same general protocol as described for Step 3, Example 116, using (R)-2-methyl-N-(3,3,3-trifluoroprop-1-en-1-yl)propane-2-sulfinamide. ESI-MS (m/z): 518.60 [M+1]⁺. ¹HNMR (CDCl₃, 400 MHz) δ 7.76 (d, J=8.2 Hz, 1H), 7.65 (d, J=8.0 Hz, 1H), 7.57 (t, J=8.0 Hz, 1H), 7.49-7.42 (m, 2H), 7.30 (s, 1H), 7.27 (s, 1H), 7.11 (d, J=8.0 Hz, 1H), 5.22-5.19 (m, 1H), 4.00-3.97 (m, 1H), 3.95-3.85 (m, 2H), 2.23-2.13 (m, 1H), 1.24 (s, 9H), 0.95 (m, 6H)

### Step 3: (R)-2,2,2-trifluoro-1-(1-isobutyl-6-(2-(trifluoromethyl)phenyl)-1H-indol-3-yl)ethan-1-amine

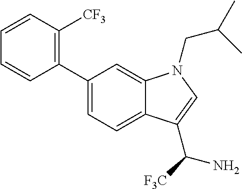 [SMILES: CC(C)Cn1cc([C@@H](N)C(F)(F)F)c2ccc(-c3ccccc3C(F)(F)F)cc21]

The title compound was prepared following the same general protocol as described for Step 4, Example 116, using (R)-2-methyl-N—((S)-2,2,2-trifluoro-1-(1-isobutyl-6-(2-(trifluoromethyl)phenyl)-1H-indol-3-yl)ethyl)propane-2-sulfinamide.

### Step 4: (R)—N-(2,2,2-trifluoro-1-(1-isobutyl-6-(2-(trifluoromethyl)phenyl)-1H-indol-3-yl)ethyl)cyclopropanesulfonamide

The title compound was prepared following the same general protocol as described for Step 5, Example 116, using (R)-2,2,2-trifluoro-1-(1-isobutyl-6-(2-(trifluoromethyl)phenyl)-1H-indol-3-yl)ethan-1-amine. ESI-MS (m/z): 518.76 [M+1]⁺.

### Example 121: (S)—N-(1-(6-bromo-1-isobutyl-1H-indol-3-yl)-2,2,2-trifluoroethyl)cyclopropanesulfonamide (306)

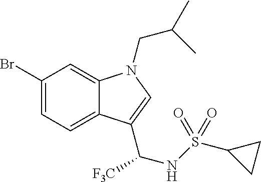 [SMILES: CC(C)Cn1cc([C@H](NS(=O)(=O)C2CC2)C(F)(F)F)c2ccc(Br)cc21]

### Step 1: (S)—N—((S)-1-(6-bromo-1-isobutyl-1H-indol-3-yl)-2,2,2-trifluoroethyl)-2-methylpropane-2-sulfinamide

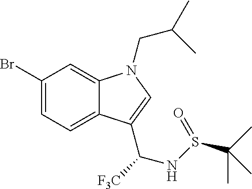 [SMILES: CC(C)Cn1cc([C@H](N[S@@](=O)C(C)(C)C)C(F)(F)F)c2ccc(Br)cc21]

The title compound was prepared following the same general protocol as described for Step 3, Example 116, using (S)-2-methyl-N-(3,3,3-trifluoroprop-1-en-1-yl)propane-2-sulfinamide and 6-bromo-1-isobutyl-1H-indole. ESI-MS (m/z): 452.53, 454.58 [M+1]⁺. ¹HNMR (CDCl₃, 400 MHz) δ 7.58-7.53 (m, 2H), 7.29-7.25 (m, 2H), 5.21-5.15 (m, 1H), 4.09-4.02 (m, 1H), 3.98-3.81 (m, 2H), 2.30-2.21 (m, 1H), 1.3 (s, 9H), 0.97 (m, 6H)

### Step 2: (S)-1-(6-bromo-1-isobutyl-1H-indol-3-yl)-2,2,2-trifluoroethan-1-amine

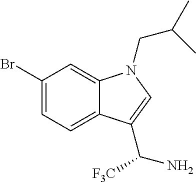 [SMILES: CC(C)Cn1cc([C@H](N)C(F)(F)F)c2ccc(Br)cc21]

The title compound was prepared following the same general protocol as described for Step 4, Example 116, using (S)—N—((S)-1-(6-bromo-1-isobutyl-1H-indol-3-yl)-2,2,2-trifluoroethyl)-2-methylpropane-2-sulfinamide.

### Step 4: N-(2,2,2-trifluoro-1-(1-isobutyl-6-(2-(trifluoromethyl)phenyl)-1H-indol-3-yl)ethyl)propane-2-sulfonamide

The title compound was prepared following the same general protocol as described for Step 5, Example 116, using (S)-1-(6-bromo-1-isobutyl-1H-indol-3-yl)-2,2,2-trifluoroethan-1-amine. ESI-MS (m/z): 452.66, 454.77 [M+1]⁺.

### Example 122: (S)—N-(2,2,2-trifluoro-1-(1-isobutyl-6-(pyridin-4-yl)-1H-indol-3-yl)ethyl)cyclopropanesulfonamide (307)

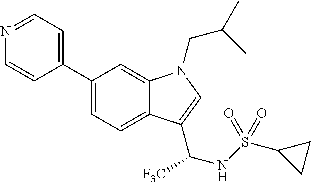 [SMILES: CC(C)Cn1cc([C@H](NS(=O)(=O)C2CC2)C(F)(F)F)c2ccc(-c3ccncc3)cc21]

The title compound was prepared following the same general protocol as described for Step 5, Example 1, using (S)—N-(1-(6-bromo-1-isobutyl-1H-indol-3-yl)-2,2,2-trifluoroethyl)cyclopropanesulfonamide. ESI-MS (m/z): 451.98 [M+1]⁺.

### Example 123: (S)—N-(2,2,2-trifluoro-1-(6-(4-fluorophenyl)-1-isobutyl-1H-indol-3-yl)ethyl)cyclopropanesulfonamide (308)

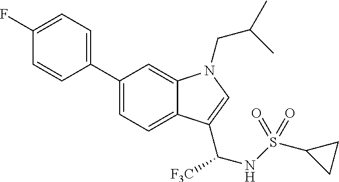 [SMILES: CC(C)Cn1cc([C@H](NS(=O)(=O)C2CC2)C(F)(F)F)c2ccc(-c3ccc(F)cc3)cc21]

The title compound was prepared following the same general protocol as described for Step 5, Example 1, using (S)—N-(1-(6-bromo-1-isobutyl-1H-indol-3-yl)-2,2,2-trifluoroethyl)cyclopropanesulfonamide and 2-(4-fluorophenyl)-4,4,5,5-tetramethyl-1,3,2-dioxaborolane. ESI-MS (m/z): 468.96[M+1]⁺.

### Example 124: (S)—N-(1-(6-(2-chlorophenyl)-1-isobutyl-1H-indol-3-yl)-2,2,2-trifluoroethyl)cyclopropanesulfonamide (311)

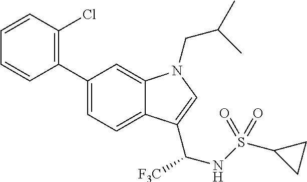 [SMILES: CC(C)Cn1cc([C@H](NS(=O)(=O)C2CC2)C(F)(F)F)c2ccc(-c3ccccc3Cl)cc21]

The title compound was prepared following the same general protocol as described for Step 5, Example 1, using (S)—N-(1-(6-bromo-1-isobutyl-1H-indol-3-yl)-2,2,2-trifluoroethyl)cyclopropanesulfonamide and (2-chlorophenyl)boronic acid. ESI-MS (m/z): 484.69 [M+1]⁺.

### Example 125: (S)—N-(2,2,2-trifluoro-1-(1-isobutyl-6-methyl-1H-indol-3-yl)ethyl)cyclopropanesulfonamide (309)

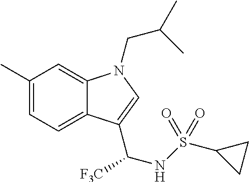 [SMILES: Cc1ccc2c([C@H](NS(=O)(=O)C3CC3)C(F)(F)F)cn(CC(C)C)c2c1]

The title compound was prepared following the same general protocol as described for Step 5, Example 1, using (S)—N-(1-(6-bromo-1-isobutyl-1H-indol-3-yl)-2,2,2-trifluoroethyl)cyclopropanesulfonamide and 2,4,6-trimethyl-1,3,5,2,4,6-trioxatriborinane. ESI-MS (m/z): 338.63 [M+1]⁺.

### Example 126: (S)—N-(1-(6-cyclopropyl-1-isobutyl-1H-indol-3-yl)-2,2,2-trifluoroethyl)cyclopropanesulfonamide (310)

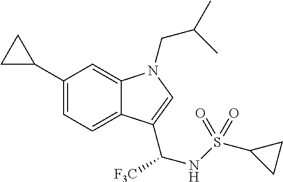 [SMILES: CC(C)Cn1cc([C@H](NS(=O)(=O)C2CC2)C(F)(F)F)c2ccc(C3CC3)cc21]

A mixture of (S)—N-(1-(6-bromo-1-isobutyl-1H-indol-3-yl)-2,2,2-trifluoroethyl)cyclopropanesulfonamide (0.10 g, 0.22 mmol), cyclopropylboronic acid (0.028 g, 0.33 mmol), K₃PO₄ (0.14 g, 0.66 mmol), Pd(OAc)₂ (0.005 g, 0.022 mmol) and Cy₃P (0.012 g, 0.044 mmol) in toluene/water (10/1) (3 mL) was degassed and heated in a 140° C. oil bath for 16 h. The solvent was removed in vacuo to obtain the crude which was purified by prep-HPLC to yield the title compound. ESI-MS (m/z): 414.68 [M+1]⁺.

### Example 128: (S)—N-(2,2,2-trifluoro-1-(5-fluoro-1-isobutyl-6-methyl-1H-indol-3-yl)ethyl)cyclopropanesulfonamide (312)

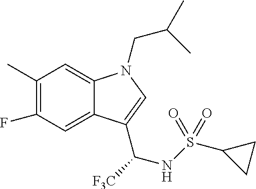 [SMILES: Cc1cc2c(cc1F)c([C@H](NS(=O)(=O)C1CC1)C(F)(F)F)cn2CC(C)C]

### Step 1: 5-fluoro-1-isobutyl-6-methyl-1H-indole

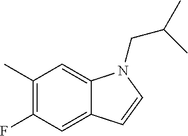

The title compound was prepared following the same general protocol as described for Step 5, Example 1, using 6-bromo-5-fluoro-1-isobutyl-1H-indole and 2,4,6-trimethyl-1,3,5,2,4,6-trioxatriborinane. ESI-MS (m/z): 205.78 [M+1]⁺.

### Step 2: (S)-2-methyl-N—((S)-2,2,2-trifluoro-1-(5-fluoro-1-isobutyl-6-methyl-1H-indol-3-yl)ethyl)propane-2-sulfinamide

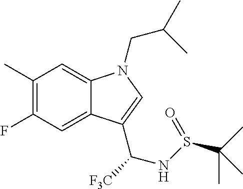 [SMILES: Cc1cc2c(cc1F)c([C@H](N[S@@](=O)C(C)(C)C)C(F)(F)F)cn2CC(C)C]

The title compound was prepared following the same general protocol as described for Step 3, Example 116, using (S)-2-methyl-N-(3,3,3-trifluoroprop-1-en-1-yl)propane-2-sulfinamide and 5-fluoro-1-isobutyl-6-methyl-1H-indole. ESI-MS (m/z): 406.55 [M+1]⁺.

### Step 3: (S)-2,2,2-trifluoro-1-(5-fluoro-1-isobutyl-6-methyl-1H-indol-3-yl)ethan-1-amine

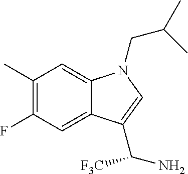 [SMILES: Cc1cc2c(cc1F)c([C@H](N)C(F)(F)F)cn2CC(C)C]

The title compound was prepared following the same general protocol as described for Step 4, Example 116, using (S)-2-methyl-N—((S)-2,2,2-trifluoro-1-(5-fluoro-1-isobutyl-6-methyl-1H-indol-3-yl)ethyl)propane-2-sulfinamide.

### Step 4: (S)—N-(2,2,2-trifluoro-1-(5-fluoro-1-isobutyl-6-methyl-1H-indol-3-yl)ethyl)cyclopropanesulfonamide

The title compound was prepared following the same general protocol as described for Step 5, Example 116, using (S)-2,2,2-trifluoro-1-(5-fluoro-1-isobutyl-6-methyl-1H-indol-3-yl)ethan-1-amine. ESI-MS (m/z): 406.60 [M+1]⁺. ¹HNMR (CDCl₃, 400 MHz) δ 7.33 (d, J=12 Hz, 1H), 7.15 (s, 1H), 7.09 (d, J=4.0 Hz, 1H), 5.29 (q, J=8.0 Hz, 1H), 5.07 (d, J=8.0 Hz, 1H), 3.84 (d, J=4.0 Hz, 2H), 2.41-2.35 (m, 4H), 2.19-2.12 (m, 1H), 1.19-1.07 (m, 2H), 0.89-0.85 (m, 8H)

### Example 129: (S)—N-(2,2,2-trifluoro-1-(5-fluoro-1-isobutyl-6-(2-(trifluoromethyl)phenyl)-1H-indol-3-yl)ethyl)cyclopropanesulfonamide (313)

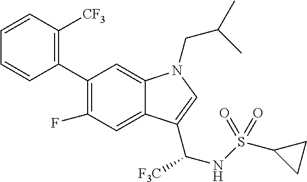 [SMILES: CC(C)Cn1cc([C@H](NS(=O)(=O)C2CC2)C(F)(F)F)c2cc(F)c(-c3ccccc3C(F)(F)F)cc21]

### Step 1: 5-fluoro-1-isobutyl-6-(2-(trifluoromethyl)phenyl)-1H-indole

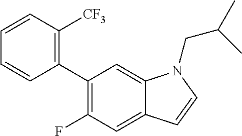 [SMILES: CC(C)Cn1ccc2cc(F)c(-c3ccccc3C(F)(F)F)cc21]

The title compound was prepared following the same general protocol as described for Step 5, Example 1, using 6-bromo-5-fluoro-1-isobutyl-1H-indole and (2-(trifluoromethyl)phenyl)boronic acid. ESI-MS (m/z): 335.87 [M+1]⁺.

### Step 2: (S)-2-methyl-N—((S)-2,2,2-trifluoro-1-(5-fluoro-1-isobutyl-6-(2-(trifluoromethyl)phenyl)-1H-indol-3-yl)ethyl)propane-2-sulfinamide

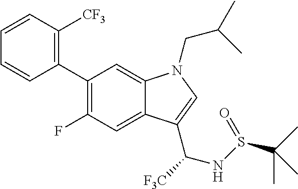 [SMILES: CC(C)Cn1cc([C@H](N[S@@](=O)C(C)(C)C)C(F)(F)F)c2cc(F)c(-c3ccccc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 5-fluoro-1-isobutyl-6-(2-(trifluoromethyl)phenyl)-1H-indole. ESI-MS (m/z): 536.55 [M+1]⁺.

### Step 3: (S)-2,2,2-trifluoro-1-(5-fluoro-1-isobutyl-6-(2-(trifluoromethyl)phenyl)-1H-indol-3-yl)ethan-1-amine

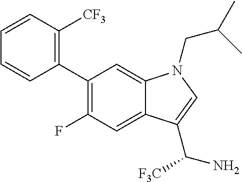 [SMILES: CC(C)Cn1cc([C@H](N)C(F)(F)F)c2cc(F)c(-c3ccccc3C(F)(F)F)cc21]

The title compound was prepared following the same general protocol as described for Step 4, Example 116, using (S)-2-methyl-N—((S)-2,2,2-trifluoro-1-(5-fluoro-1-isobutyl-6-(2-(trifluoromethyl)phenyl)-1H-indol-3-yl)ethyl)propane-2-sulfinamide.

### Step 4: (S)—N-(2,2,2-trifluoro-1-(5-fluoro-1-isobutyl-6-(2-(trifluoromethyl)phenyl)-1H-indol-3-yl)ethyl)cyclopropanesulfonamide

The title compound was prepared following the same general protocol as described for Step 5, Example 116, using (S)-2,2,2-trifluoro-1-(5-fluoro-1-isobutyl-6-(2-(trifluoromethyl)phenyl)-1H-indol-3-yl)ethan-1-amine. ESI-MS (m/z): 536.67 [M+1]⁺.

### Example 130: (S)—N-(1-(6-(2-chlorophenyl)-5-fluoro-1-isobutyl-1H-indol-3-yl)-2,2,2-trifluoroethyl)cyclopropanesulfonamide (314)

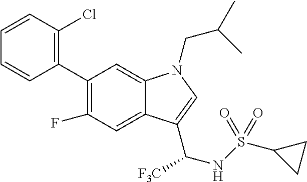 [SMILES: CC(C)Cn1cc([C@H](NS(=O)(=O)C2CC2)C(F)(F)F)c2cc(F)c(-c3ccccc3Cl)cc21]

### Step 1: 6-(2-chlorophenyl)-5-fluoro-1-isobutyl-1H-indole

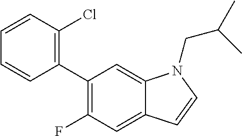 [SMILES: CC(C)Cn1ccc2cc(F)c(-c3ccccc3Cl)cc21]

The title compound was prepared following the same general protocol as described for Step 5, Example 1, using 6-bromo-5-fluoro-1-isobutyl-1H-indole and (2-chlorophenyl)boronic acid. ESI-MS (m/z): 302.34 [M+1]⁺.

### Step 2: (S)—N—((S)-1-(6-(2-chlorophenyl)-5-fluoro-1-isobutyl-1H-indol-3-yl)-2,2,2-trifluoroethyl)-2-methylpropane-2-sulfinamide

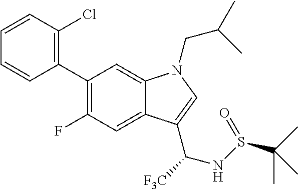 [SMILES: CC(C)Cn1cc([C@H](N[S@@](=O)C(C)(C)C)C(F)(F)F)c2cc(F)c(-c3ccccc3Cl)cc21]

The title compound was prepared following the same general protocol as described for Step 3, Example 116, using (S)-2-methyl-N-(3,3,3-trifluoroprop-1-en-1-yl)propane-2-sulfinamide and 6-(2-chlorophenyl)-5-fluoro-1-isobutyl-1H-indole. ESI-MS (m/z): 502.55 [M+1]⁺.

### Step 3: (S)-1-(6-(2-chlorophenyl)-5-fluoro-1-isobutyl-1H-indol-3-yl)-2,2,2-trifluoroethan-1-amine

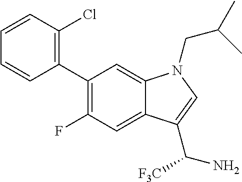 [SMILES: CC(C)Cn1cc([C@H](N)C(F)(F)F)c2cc(F)c(-c3ccccc3Cl)cc21]

The title compound was prepared following the same general protocol as described for Step 4, Example 116, using (S)—N—((S)-1-(6-(2-chlorophenyl)-5-fluoro-1-isobutyl-1H-indol-3-yl)-2,2,2-trifluoroethyl)-2-methylpropane-2-sulfinamide.

### Step 4: (S)—N-(1-(6-(2-chlorophenyl)-5-fluoro-1-isobutyl-1H-indol-3-yl)-2,2,2-trifluoroethyl)cyclopropanesulfonamide

The title compound was prepared following the same general protocol as described for Step 5, Example 116, using (S)-1-(6-(2-chlorophenyl)-5-fluoro-1-isobutyl-1H-indol-3-yl)-2,2,2-trifluoroethan-1-amine. ESI-MS (m/z): 502.62 [M+1]⁺. ¹HNMR (CDCl₃, 400 MHz) δ 7.52-48 (m, 2H), 7.40-7.32 (m, 3H), 7.27-7.24 (m, 2H), 5.36 (q, J=8.0 Hz, 1H), 4.96 (d, J=8.0 Hz, 1H), 3.88 (d, J=8.0 Hz, 2H), 2.49-2.43 (m, 1H), 2.20-2.14 (m, 1H), 1.27-1.22 (m, 1H), 1.17-1.12 (m, 1H), 1.01-0.95 (m, 8H)

### Example 131: (S)—N-(1-(6-cyclopropyl-5-fluoro-1-isobutyl-1H-indol-3-yl)-2,2,2-trifluoroethyl)cyclopropanesulfonamide (315)

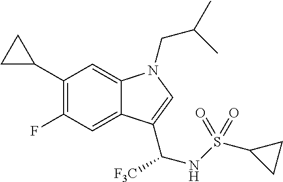 [SMILES: CC(C)Cn1cc([C@H](NS(=O)(=O)C2CC2)C(F)(F)F)c2cc(F)c(C3CC3)cc21]

### Step 1: 6-cyclopropyl-5-fluoro-1-isobutyl-1H-indole

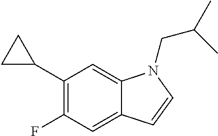 [SMILES: CC(C)Cn1ccc2cc(F)c(C3CC3)cc21]

The title compound was prepared following the same general protocol as described for Example 126, using 6-bromo-5-fluoro-1-isobutyl-1H-indole and cyclopropylboronic acid. ESI-MS (m/z): 231.85 [M+1]⁺.

### Step 2: (S)—N—((S)-1-(6-cyclopropyl-5-fluoro-1-isobutyl-1H-indol-3-yl)-2,2,2-trifluoroethyl)-2-methylpropane-2-sulfinamide

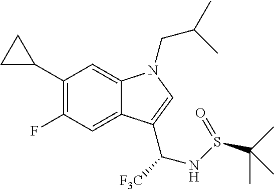 [SMILES: CC(C)Cn1cc([C@H](N[S@@](=O)C(C)(C)C)C(F)(F)F)c2cc(F)c(C3CC3)cc21]

The title compound was prepared following the same general protocol as described for Step 3, Example 116, using (S)-2-methyl-N-(3,3,3-trifluoroprop-1-en-1-yl)propane-2-sulfinamide and 6-cyclopropyl-5-fluoro-1-isobutyl-1H-indole. ESI-MS (m/z): 432.55 [M+1]⁺.

### Step 3: (S)-1-(6-cyclopropyl-5-fluoro-1-isobutyl-1H-indol-3-yl)-2,2,2-trifluoroethan-1-amine

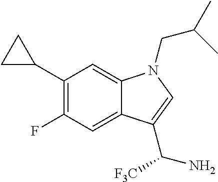 [SMILES: CC(C)Cn1cc([C@H](N)C(F)(F)F)c2cc(F)c(C3CC3)cc21]

The title compound was prepared following the same general protocol as described for Step 4, Example 116, using (S)—N—((S)-1-(6-cyclopropyl-5-fluoro-1-isobutyl-1H-indol-3-yl)-2,2,2-trifluoroethyl)-2-methylpropane-2-sulfinamide.

### Step 4: (S)—N-(1-(6-cyclopropyl-5-fluoro-1-isobutyl-1H-indol-3-yl)-2,2,2-trifluoroethyl)cyclopropanesulfonamide

The title compound was prepared following the same general protocol as described for Step 5, Example 116, using (S)-1-(6-cyclopropyl-5-fluoro-1-isobutyl-1H-indol-3-yl)-2,2,2-trifluoroethan-1-amine. ESI-MS (m/z): 432.68 [M+1]⁺. ¹HNMR (CDCl₃, 400 MHz) δ 7.33 (d, J=8.0 Hz, 1H), 7.15 (s, 1H), 6.85 (d, J=4.0 Hz, 1H), 5.29 (q, J=8.0 Hz, 1H), 5.02 (d, J=8.0 Hz, 1H), 3.82 (d, J=8.0 Hz, 2H), 2.41-2.35 (m, 1H), 2.19-2.09 (m, 2H), 1.17-1.11 (m, 2H), 1.01-0.97 (m, 2H), 0.88-0.86 (m, 8H), 0.74-0.70 (m, 2H)

### Example 132: (S)—N-(2,2,2-trifluoro-1-(5-fluoro-1-isobutyl-6-isopropyl-1H-indol-3-yl)ethyl)cyclopropanesulfonamide (334)

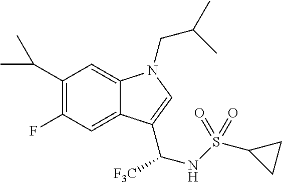 [SMILES: CC(C)Cn1cc([C@H](NS(=O)(=O)C2CC2)C(F)(F)F)c2cc(F)c(C(C)C)cc21]

### Step 1: 5-fluoro-1-isobutyl-6-(prop-1-en-2-yl)-1H-indole

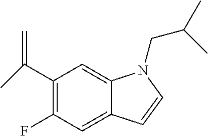 [SMILES: C=C(C)c1cc2c(ccn2CC(C)C)cc1F]

The title compound was prepared following the same general protocol as described for Example 126, using 6-bromo-5-fluoro-1-isobutyl-1H-indole and 4,4,5,5-tetramethyl-2-(prop-1-en-2-yl)-1,3,2-dioxaborolane. ESI-MS (m/z): 231.76 [M+1]⁺.

### Step 2: 5-fluoro-1-isobutyl-6-isopropyl-1H-indole

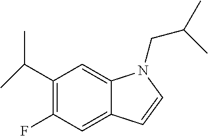 [SMILES: CC(C)Cn1ccc2cc(F)c(C(C)C)cc21]

The title compound was prepared following the same general protocol as described for Example 20, using 5-fluoro-1-isobutyl-6-(prop-1-en-2-yl)-1H-indole. ESI-MS (m/z): 233.89 [M+1]⁺.

### Step 3: (S)-2-methyl-N—((S)-2,2,2-trifluoro-1-(5-fluoro-1-isobutyl-6-isopropyl-1H-indol-3-yl)ethyl)propane-2-sulfinamide

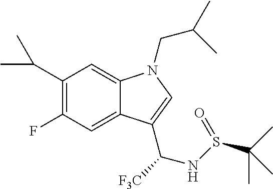 [SMILES: CC(C)Cn1cc([C@H](N[S@@](=O)C(C)(C)C)C(F)(F)F)c2cc(F)c(C(C)C)cc21]

The title compound was prepared following the same general protocol as described for Step 3, Example 116, using (S)-2-methyl-N-(3,3,3-trifluoroprop-1-en-1-yl)propane-2-sulfinamide and 5-fluoro-1-isobutyl-6-isopropyl-1H-indole. ESI-MS (m/z): 510.78 [M+1]⁺.

### Step 4: (S)-2,2,2-trifluoro-1-(5-fluoro-1-isobutyl-6-isopropyl-1H-indol-3-yl)ethan-1-amine

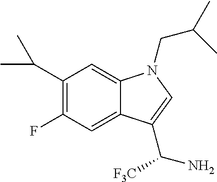 [SMILES: CC(C)Cn1cc([C@H](N)C(F)(F)F)c2cc(F)c(C(C)C)cc21]

The title compound was prepared following the same general protocol as described for Step 4, Example 116, using (S)-2-methyl-N—((S)-2,2,2-trifluoro-1-(5-fluoro-1-isobutyl-6-isopropyl-1H-indol-3-yl)ethyl)propane-2-sulfinamide.

### Step 5: (S)—N-(2,2,2-trifluoro-1-(5-fluoro-1-isobutyl-6-isopropyl-1H-indol-3-yl)ethyl)cyclopropanesulfonamide

The title compound was prepared following the same general protocol as described for Step 5, Example 116, using (S)-2,2,2-trifluoro-1-(5-fluoro-1-isobutyl-6-isopropyl-1H-indol-3-yl)ethan-1-amine. ESI-MS (m/z): 510.90 [M+1]⁺.

### Example 133: (S)—N-(2,2,2-trifluoro-1-(5-fluoro-1-neopentyl-6-(2-(trifluoromethyl)phenyl)-1H-indol-3-yl)ethyl)cyclopropanesulfonamide (319)

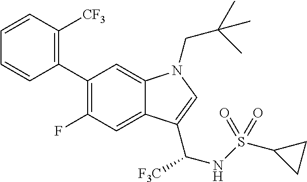 [SMILES: CC(C)(C)Cn1cc([C@H](NS(=O)(=O)C2CC2)C(F)(F)F)c2cc(F)c(-c3ccccc3C(F)(F)F)cc21]

### Step 1: 6-bromo-5-fluoro-1-neopentyl-1H-indole

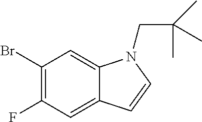 [SMILES: CC(C)(C)Cn1ccc2cc(F)c(Br)cc21]

The title compound was prepared following the same general protocol as described for Example 34, using 6-bromo-5-fluoro-1H-indole and 1-iodo-2,2-dimethylpropane. ESI-MS (m/z): 283.77, 285.79 [M+1]⁺.

### Step 2: 5-fluoro-1-neopentyl-6-(2-(trifluoromethyl)phenyl)-1H-indole

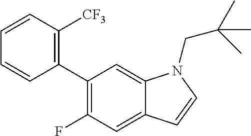 [SMILES: CC(C)(C)Cn1ccc2cc(F)c(-c3ccccc3C(F)(F)F)cc21]

The title compound was prepared following the same general protocol as described for Step 5, Example 1, using 6-bromo-5-fluoro-1-neopentyl-1H-indole and (2-(trifluoromethyl)phenyl)boronic acid. ESI-MS (m/z): 349.95 [M+1]⁺.

### Step 3: (S)-2-methyl-N—((S)-2,2,2-trifluoro-1-(5-fluoro-1-neopentyl-6-(2-(trifluoromethyl)phenyl)-1H-indol-3-yl)ethyl)propane-2-sulfinamide

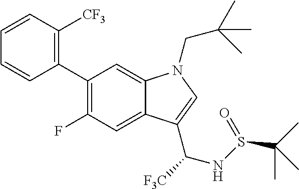 [SMILES: CC(C)(C)Cn1cc([C@H](N[S@@](=O)C(C)(C)C)C(F)(F)F)c2cc(F)c(-c3ccccc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 5-fluoro-1-neopentyl-6-(2-(trifluoromethyl)phenyl)-1H-indole. ESI-MS (m/z): 550.55 [M+1]⁺.

### Step 4: (S)-2,2,2-trifluoro-1-(5-fluoro-1-neopentyl-6-(2-(trifluoromethyl)phenyl)-1H-indol-3-yl)ethan-1-amine

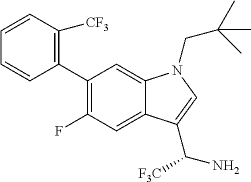 [SMILES: CC(C)(C)Cn1cc([C@H](N)C(F)(F)F)c2cc(F)c(-c3ccccc3C(F)(F)F)cc21]

The title compound was prepared following the same general protocol as described for Step 4, Example 116, using (S)-2-methyl-N—((S)-2,2,2-trifluoro-1-(5-fluoro-1-neopentyl-6-(2-(trifluoromethyl)phenyl)-1H-indol-3-yl)ethyl)propane-2-sulfinamide.

### Step 5: (S)—N-(2,2,2-trifluoro-1-(5-fluoro-1-neopentyl-6-(2-(trifluoromethyl)phenyl)-1H-indol-3-yl)ethyl)cyclopropanesulfonamide

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine. ESI-MS (m/z): 550.64 [M+1]⁺. ¹H NMR (400 MHz, d₆-DMSO) δ 8.64 (s, 1H), 7.86 (d, J=7.6 Hz, 1H), 7.78-7.69 (m, 3H), 7.66 (t, J=7.7 Hz, 1H), 7.54 (d, J=6.2 Hz, 1H), 7.46 (d, J=7.5 Hz, 1H), 5.50 (s, 1H), 4.08 (d, J=14.2 Hz, 1H), 3.93 (dd, J=14.2, 3.2 Hz, 1H), 2.32 (dd, J=7.5, 4.2 Hz, 1H), 0.98-0.60 (m, 13H).

### Example 134: (S)—N-(2,2,2-trifluoro-1-(5-fluoro-6-methyl-1-neopentyl-1H-indol-3-yl)ethyl)cyclopropanesulfonamide (316)

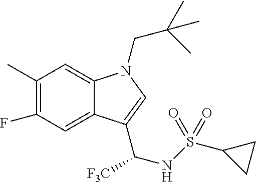 [SMILES: Cc1cc2c(cc1F)c([C@H](NS(=O)(=O)C1CC1)C(F)(F)F)cn2CC(C)(C)C]

### Step 1: 5-fluoro-1-neopentyl-6-(2-(trifluoromethyl)phenyl)-1H-indole

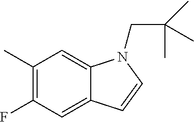 [SMILES: Cc1cc2c(ccn2CC(C)(C)C)cc1F]

The title compound was prepared following the same general protocol as described for Step 5, Example 1, using 6-bromo-5-fluoro-1-neopentyl-1H-indole and 2,4,6-trimethyl-1,3,5,2,4,6-trioxatriborinane. ESI-MS (m/z): 219.87 [M+1]⁺.

### Step 2: (S)-2-methyl-N—((S)-2,2,2-trifluoro-1-(5-fluoro-6-methyl-1-neopentyl-1H-indol-3-yl)ethyl)propane-2-sulfinamide

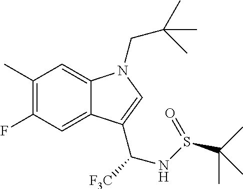 [SMILES: Cc1cc2c(cc1F)c([C@H](N[S@@](=O)C(C)(C)C)C(F)(F)F)cn2CC(C)(C)C]

The title compound was prepared following the same general protocol as described for Step 3, Example 116, using (S)-2-methyl-N-(3,3,3-trifluoroprop-1-en-1-yl)propane-2-sulfinamide and 5-fluoro-6-methyl-1-neopentyl-1H-indole. ESI-MS (m/z): 420.57 [M+1]⁺.

### Step 3: (S)-2,2,2-trifluoro-1-(5-fluoro-6-methyl-1-neopentyl-1H-indol-3-yl)ethan-1-amine

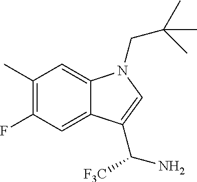 [SMILES: Cc1cc2c(cc1F)c([C@H](N)C(F)(F)F)cn2CC(C)(C)C]

The title compound was prepared following the same general protocol as described for Step 4, Example 116, using (S)-2-methyl-N—((S)-2,2,2-trifluoro-1-(5-fluoro-6-methyl-1-neopentyl-1H-indol-3-yl)ethyl)propane-2-sulfinamide.

### Step 4: (S)—N-(2,2,2-trifluoro-1-(5-fluoro-6-methyl-1-neopentyl-1H-indol-3-yl)ethyl)cyclopropanesulfonamide

The title compound was prepared following the same general protocol as described for Step 5, Example 116, using (S)-2,2,2-trifluoro-1-(5-fluoro-6-methyl-1-neopentyl-1H-indol-3-yl)ethan-1-amine. ESI-MS (m/z): 420.63 [M+1]⁺. ¹HNMR (CDCl₃, 400 MHz) δ 7.31 (d, J=8.0 Hz, 1H), 7.14 (s, 1H), 7.10 (d, J=8.0 Hz, 1H), 5.29 (q, J=8.0 Hz, 1H), 5.11 (d, J=8.0 Hz, 1H), 3.83 (s, 2H), 2.41-2.35 (m, 4H), 1.37-1.02 (m, 2H), 0.99 (s, 9H), 0.92-0.82 (m, 2H)

### Example 135: (S)—N-(1-(6-(2-chlorophenyl)-5-fluoro-1-neopentyl-1H-indol-3-yl)-2,2,2-trifluoroethyl)cyclopropanesulfonamide (331)

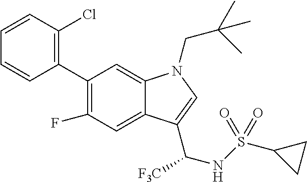 [SMILES: CC(C)(C)Cn1cc([C@H](NS(=O)(=O)C2CC2)C(F)(F)F)c2cc(F)c(-c3ccccc3Cl)cc21]

### Step 1: 6-(2-chlorophenyl)-5-fluoro-1-neopentyl-1H-indole

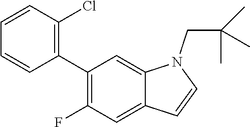 [SMILES: CC(C)(C)Cn1ccc2cc(F)c(-c3ccccc3Cl)cc21]

The title compound was prepared following the same general protocol as described for Step 5, Example 1, using 6-bromo-5-fluoro-1-neopentyl-1H-indole and (2-chlorophenyl)boronic acid. ESI-MS (m/z): 316.41 [M+1]⁺.

### Step 2: (S)—N—((S)-1-(6-(2-chlorophenyl)-5-fluoro-1-neopentyl-1H-indol-3-yl)-2,2,2-trifluoroethyl)-2-methylpropane-2-sulfinamide

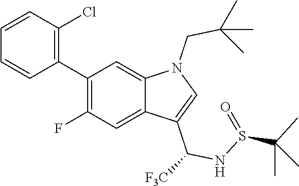 [SMILES: CC(C)(C)Cn1cc([C@H](N[S@@](=O)C(C)(C)C)C(F)(F)F)c2cc(F)c(-c3ccccc3Cl)cc21]

The title compound was prepared following the same general protocol as described for Step 3, Example 116, using (S)-2-methyl-N-(3,3,3-trifluoroprop-1-en-1-yl)propane-2-sulfinamide and 6-(2-chlorophenyl)-5-fluoro-1-neopentyl-1H-indole. ESI-MS (m/z): 516.62 [M+1]⁺.

### Step 3: (S)-1-(6-(2-chlorophenyl)-5-fluoro-1-neopentyl-1H-indol-3-yl)-2,2,2-trifluoroethan-1-amine

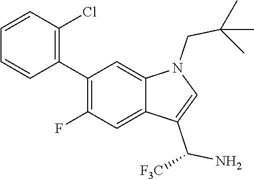 [SMILES: CC(C)(C)Cn1cc([C@H](N)C(F)(F)F)c2cc(F)c(-c3ccccc3Cl)cc21]

The title compound was prepared following the same general protocol as described for Step 4, Example 116, using (S)—N—((S)-1-(6-(2-chlorophenyl)-5-fluoro-1-neopentyl-1H-indol-3-yl)-2,2,2-trifluoroethyl)-2-methylpropane-2-sulfinamide.

### Step 4: (S)—N-(1-(6-(2-chlorophenyl)-5-fluoro-1-neopentyl-1H-indol-3-yl)-2,2,2-trifluoroethyl)cyclopropanesulfonamide

The title compound was prepared following the same general protocol as described for Step 5, Example 116, using (S)-1-(6-(2-chlorophenyl)-5-fluoro-1-neopentyl-1H-indol-3-yl)-2,2,2-trifluoroethan-1-amine. ESI-MS (m/z): 516.80 [M+1]⁺.

### Example 136: (S)—N-(1-(6-cyclopropyl-5-fluoro-1-neopentyl-1H-indol-3-yl)-2,2,2-trifluoroethyl)cyclopropanesulfonamide (327)

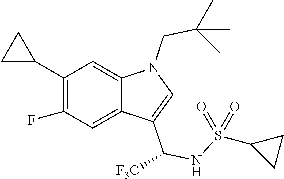 [SMILES: CC(C)(C)Cn1cc([C@H](NS(=O)(=O)C2CC2)C(F)(F)F)c2cc(F)c(C3CC3)cc21]

### Step 1: 6-cyclopropyl-5-fluoro-1-neopentyl-1H-indole

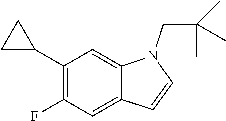 [SMILES: CC(C)(C)Cn1ccc2cc(F)c(C3CC3)cc21]

The title compound was prepared following the same general protocol as described for Step 5, Example 1, using 6-bromo-5-fluoro-1-neopentyl-1H-indole and cyclopropylboronic acid. ESI-MS (m/z): 245.78 [M+1]⁺.

### Step 2: (S)—N—((S)-1-(6-cyclopropyl-5-fluoro-1-neopentyl-1H-indol-3-yl)-2,2,2-trifluoroethyl)-2-methylpropane-2-sulfinamide

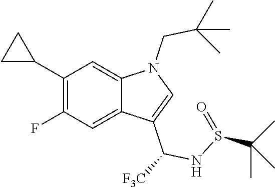 [SMILES: CC(C)(C)Cn1cc([C@H](N[S@@](=O)C(C)(C)C)C(F)(F)F)c2cc(F)c(C3CC3)cc21]

The title compound was prepared following the same general protocol as described for Step 3, Example 116, using (S)-2-methyl-N-(3,3,3-trifluoroprop-1-en-1-yl)propane-2-sulfinamide and 6-cyclopropyl-5-fluoro-1-neopentyl-1H-indole. ESI-MS (m/z): 446.63 [M+1]⁺.

### Step 3: (S)-1-(6-cyclopropyl-5-fluoro-1-neopentyl-1H-indol-3-yl)-2,2,2-trifluoroethan-1-amine

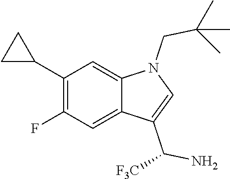 [SMILES: CC(C)(C)Cn1cc([C@H](N)C(F)(F)F)c2cc(F)c(C3CC3)cc21]

The title compound was prepared following the same general protocol as described for Step 4, Example 116, using (S)—N—((S)-1-(6-cyclopropyl-5-fluoro-1-neopentyl-1H-indol-3-yl)-2,2,2-trifluoroethyl)-2-methylpropane-2-sulfinamide.

### Step 4: (S)—N-(1-(6-cyclopropyl-5-fluoro-1-neopentyl-1H-indol-3-yl)-2,2,2-trifluoroethyl)cyclopropanesulfonamide

The title compound was prepared following the same general protocol as described for Step 5, Example 116, using (S)-1-(6-cyclopropyl-5-fluoro-1-neopentyl-1H-indol-3-yl)-2,2,2-trifluoroethan-1-amine. ESI-MS (m/z): 446.76 [M+1]⁺. ¹HNMR (CDCl₃, 400 MHz) δ 7.32 (d, J=12 Hz, 1H), 7.13 (s, 1H), 6.87 (d, J=4.0 Hz, 1H), 5.29 (q, J=8.0 Hz, 1H), 4.99 (d, J=8.0 Hz, 1H), 3.81 (s, 2H), 2.43-2.36 (m, 1H), 2.19-2.12 (m, 1H), 1.22-1.14 (m, 2H), 1.03-0.96 (m, 11H), 0.88-0.86 (m, 2H), 0.71-0.67 (m, 2H)

### Example 137: (S)—N-(2,2,2-trifluoro-1-(5-fluoro-6-isopropyl-1-neopentyl-1H-indol-3-yl)ethyl)cyclopropanesulfonamide (336)

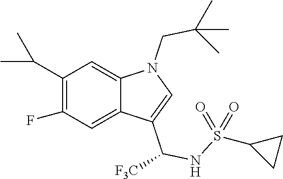 [SMILES: CC(C)c1cc2c(cc1F)c([C@H](NS(=O)(=O)C1CC1)C(F)(F)F)cn2CC(C)(C)C]

### Step 1: 5-fluoro-1-neopentyl-6-(prop-1-en-2-yl)-1H-indole

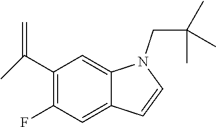 [SMILES: C=C(C)c1cc2c(ccn2CC(C)(C)C)cc1F]

The title compound was prepared following the same general protocol as described for Example 126, using 6-bromo-5-fluoro-1-neopentyl-1H-indole and 4,4,5,5-tetramethyl-2-(prop-1-en-2-yl)-1,3,2-dioxaborolane. ESI-MS (m/z): 245.79 [M+1]⁺.

### Step 2: 5-fluoro-6-isopropyl-1-neopentyl-1H-indole

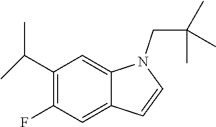 [SMILES: CC(C)c1cc2c(ccn2CC(C)(C)C)cc1F]

The title compound was prepared following the same general protocol as described for Example 20, using 5-fluoro-1-neopentyl-6-(prop-1-en-2-yl)-1H-indole. ESI-MS (m/z): 247.93 [M+1]⁺.

### Step 3: (S)-2-methyl-N—((S)-2,2,2-trifluoro-1-(5-fluoro-6-isopropyl-1-neopentyl-1H-indol-3-yl)ethyl)propane-2-sulfinamide

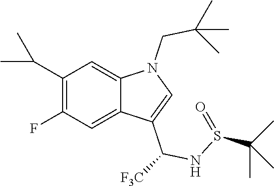 [SMILES: CC(C)c1cc2c(cc1F)c([C@H](N[S@@](=O)C(C)(C)C)C(F)(F)F)cn2CC(C)(C)C]

The title compound was prepared following the same general protocol as described for Step 3, Example 116, using (S)-2-methyl-N-(3,3,3-trifluoroprop-1-en-1-yl)propane-2-sulfinamide and 5-fluoro-6-isopropyl-1-neopentyl-1H-indole. ESI-MS (m/z): 448.56 [M+1]⁺.

### Step 4: (S)-2,2,2-trifluoro-1-(5-fluoro-6-isopropyl-1-neopentyl-1H-indol-3-yl)ethan-1-amine

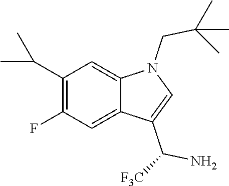 [SMILES: CC(C)c1cc2c(cc1F)c([C@H](N)C(F)(F)F)cn2CC(C)(C)C]

The title compound was prepared following the same general protocol as described for Step 4, Example 116, using (S)-2-methyl-N—((S)-2,2,2-trifluoro-1-(5-fluoro-6-isopropyl-1-neopentyl-1H-indol-3-yl)ethyl)propane-2-sulfinamide.

### Step 5: (S)—N-(2,2,2-trifluoro-1-(5-fluoro-6-isopropyl-1-neopentyl-1H-indol-3-yl)ethyl)cyclopropanesulfonamide

The title compound was prepared following the same general protocol as described for Step 5, Example 116, using (S)-2,2,2-trifluoro-1-(5-fluoro-6-isopropyl-1-neopentyl-1H-indol-3-yl)ethan-1-amine. ESI-MS (m/z): 448.78 [M+1]⁺. ¹HNMR (CDCl₃, 400 MHz) δ 7.43 (s, 1H), 7.33-7.27 (m, 1H), 7.11 (s, 1H), 5.29 (q, J=8.0 Hz, 1H), 4.99 (d, J=8.0 Hz, 1H), 3.84 (s, 2H), 2.41-2.37 (m, 1H), 1.48-0.88 (m, 20H)

### Example 138: (S)—N-(2,2,2-trifluoro-1-(1-neopentyl-6-(2-(trifluoromethyl)phenyl)-1H-indol-3-yl)ethyl)cyclopropanesulfonamide (322)

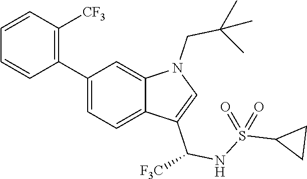 [SMILES: CC(C)(C)Cn1cc([C@H](NS(=O)(=O)C2CC2)C(F)(F)F)c2ccc(-c3ccccc3C(F)(F)F)cc21]

### Step 1: 6-bromo-1-neopentyl-1H-indole

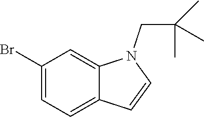

The title compound was prepared following the same general protocol as described for Example 34, using 6-bromoindole and 1-iodo-2,2-dimethylpropane. ESI-MS (m/z): 265.68, 267.70 [M+1]⁺.

### Step 2: 1-neopentyl-6-(2-(trifluoromethyl)phenyl)-1H-indole

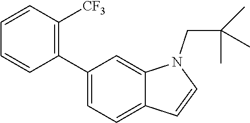 [SMILES: CC(C)(C)Cn1ccc2ccc(-c3ccccc3C(F)(F)F)cc21]

The title compound was prepared following the same general protocol as described for Step 5, Example 1, using 6-bromo-1-neopentyl-1H-indole and (2-(trifluoromethyl)phenyl)boronic acid. ESI-MS (m/z): 331.89 [M+1]⁺.

### Step 3: (S)-2-methyl-N—((S)-2,2,2-trifluoro-1-(1-neopentyl-6-(2-(trifluoromethyl)phenyl)-1H-indol-3-yl)ethyl)propane-2-sulfinamide

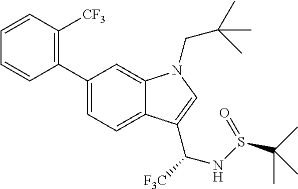 [SMILES: CC(C)(C)Cn1cc([C@H](N[S@@](=O)C(C)(C)C)C(F)(F)F)c2ccc(-c3ccccc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 1-neopentyl-6-(2-(trifluoromethyl)phenyl)-1H-indole. ESI-MS (m/z): 532.59 [M+1]⁺.

### Step 4: (S)-2,2,2-trifluoro-1-(1-neopentyl-6-(2-(trifluoromethyl)phenyl)-1H-indol-3-yl)ethan-1-amine

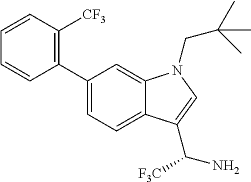 [SMILES: CC(C)(C)Cn1cc([C@H](N)C(F)(F)F)c2ccc(-c3ccccc3C(F)(F)F)cc21]

The title compound was prepared following the same general protocol as described for Step 4, Example 116, using (S)-2-methyl-N—((S)-2,2,2-trifluoro-1-(1-neopentyl-6-(2-(trifluoromethyl)phenyl)-1H-indol-3-yl)ethyl)propane-2-sulfinamide.

### Step 5: (S)—N-(2,2,2-trifluoro-1-(1-neopentyl-6-(2-(trifluoromethyl)phenyl)-1H-indol-3-yl)ethyl)cyclopropanesulfonamide

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine. ESI-MS (m/z): 532.79 [M+1]⁺.

### Example 139: (S)—N-(1-(6-(2-chlorophenyl)-1-neopentyl-1H-indol-3-yl)-2,2,2-trifluoroethyl)cyclopropanesulfonamide (332)

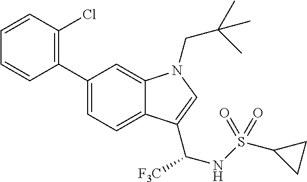 [SMILES: CC(C)(C)Cn1cc([C@H](NS(=O)(=O)C2CC2)C(F)(F)F)c2ccc(-c3ccccc3Cl)cc21]

### Step 1: 6-(2-chlorophenyl)-1-neopentyl-1H-indole

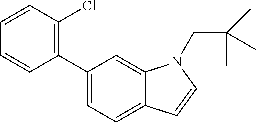 [SMILES: CC(C)(C)Cn1ccc2ccc(-c3ccccc3Cl)cc21]

The title compound was prepared following the same general protocol as described for Step 5, Example 1, using 6-bromo-1-neopentyl-1H-indole and (2-chlorophenyl)boronic acid. ESI-MS (m/z): 298.35 [M+1]⁺.

### Step 2: (S)—N—((S)-1-(6-(2-chlorophenyl)-1-neopentyl-1H-indol-3-yl)-2,2,2-trifluoroethyl)-2-methylpropane-2-sulfinamide

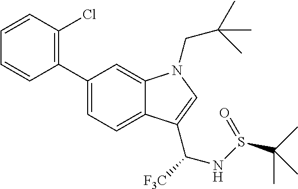 [SMILES: CC(C)(C)Cn1cc([C@H](N[S@@](=O)C(C)(C)C)C(F)(F)F)c2ccc(-c3ccccc3Cl)cc21]

The title compound was prepared following the same general protocol as described for Step 3, Example 116, using (S)-2-methyl-N-(3,3,3-trifluoroprop-1-en-1-yl)propane-2-sulfinamide and 6-(2-chlorophenyl)-1-neopentyl-1H-indole. ESI-MS (m/z): 498.55 [M+1]⁺.

### Step 3: (S)-1-(6-(2-chlorophenyl)-1-neopentyl-1H-indol-3-yl)-2,2,2-trifluoroethan-1-amine

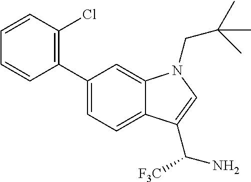 [SMILES: CC(C)(C)Cn1cc([C@H](N)C(F)(F)F)c2ccc(-c3ccccc3Cl)cc21]

The title compound was prepared following the same general protocol as described for Step 4, Example 116, using (S)—N—((S)-1-(6-(2-chlorophenyl)-1-neopentyl-1H-indol-3-yl)-2,2,2-trifluoroethyl)-2-methylpropane-2-sulfinamide.

### Step 4: (S)—N-(1-(6-(2-chlorophenyl)-1-neopentyl-1H-indol-3-yl)-2,2,2-trifluoroethyl)cyclopropanesulfonamide

The title compound was prepared following the same general protocol as described for Step 5, Example 116, using (S)-1-(6-(2-chlorophenyl)-1-neopentyl-1H-indol-3-yl)-2,2,2-trifluoroethan-1-amine. ESI-MS (m/z): 498.71 [M+1]⁺.

### Example 140: (S)—N-(2,2,2-trifluoro-1-(1-isobutyl-6-isopropyl-1H-indol-3-yl)ethyl)cyclopropanesulfonamide (333)

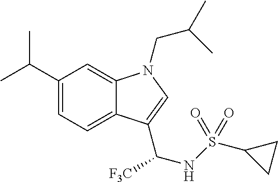 [SMILES: CC(C)Cn1cc([C@H](NS(=O)(=O)C2CC2)C(F)(F)F)c2ccc(C(C)C)cc21]

### Step 1: (E)-2-(4-isopropyl-2-nitrophenyl)-N,N-dimethylethen-1-amine

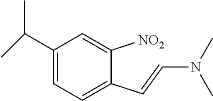 [SMILES: CC(C)c1ccc(/C=C/N(C)C)c([N+](=O)[O-])c1]

The title compound was prepared following the same general protocol as described for Step 1, Example 63, using 4-isopropyl-1-methyl-2-nitrobenzene.

### Step 2: 6-isopropyl-1H-indole

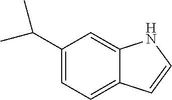

The title compound was prepared following the same general protocol as described for Step 2, Example 63, using (E)-2-(4-isopropyl-2-nitrophenyl)-N,N-dimethylethen-1-amine.

### Step 3: 1-isobutyl-6-isopropyl-1H-indole

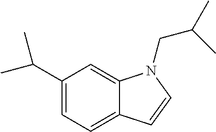 [SMILES: CC(C)Cn1ccc2ccc(C(C)C)cc21]

The title compound was prepared following the same general protocol as described for Example 34, using 6-isopropyl-1H-indole and 1-bromo-2-methylpropane. ESI-MS (m/z): 215.81 [M+1]⁺.

### Step 4: (S)-2-methyl-N—((S)-2,2,2-trifluoro-1-(1-isobutyl-6-isopropyl-1H-indol-3-yl)ethyl)propane-2-sulfinamide

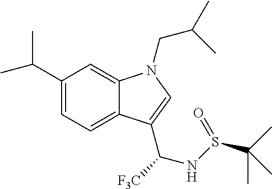 [SMILES: CC(C)Cn1cc([C@H](N[S@@](=O)C(C)(C)C)C(F)(F)F)c2ccc(C(C)C)cc21]

The title compound was prepared following the same general protocol as described for Step 3, Example 116, using (S)-2-methyl-N-(3,3,3-trifluoroprop-1-en-1-yl)propane-2-sulfinamide and 1-isobutyl-6-isopropyl-1H-indole. ESI-MS (m/z): 416.63 [M+1]⁺.

### Step 5: (S)-2,2,2-trifluoro-1-(1-isobutyl-6-isopropyl-1H-indol-3-yl)ethan-1-amine

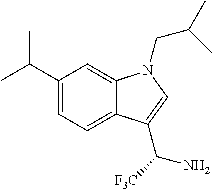 [SMILES: CC(C)Cn1cc([C@H](N)C(F)(F)F)c2ccc(C(C)C)cc21]

The title compound was prepared following the same general protocol as described for Step 4, Example 116, using (S)-2-methyl-N—((S)-2,2,2-trifluoro-1-(1-isobutyl-6-isopropyl-1H-indol-3-yl)ethyl)propane-2-sulfinamide.

### Step 6: (S)—N-(2,2,2-trifluoro-1-(1-isobutyl-6-isopropyl-1H-indol-3-yl)ethyl)cyclopropanesulfonamide

The title compound was prepared following the same general protocol as described for Step 5, Example 116, using (S)-2,2,2-trifluoro-1-(1-isobutyl-6-isopropyl-1H-indol-3-yl)ethan-1-amine. ESI-MS (m/z): 416.81 [M+1]⁺.

### Example 141: (S)—N-(2,2,2-trifluoro-1-(6-isopropyl-1-neopentyl-1H-indol-3-yl)ethyl)cyclopropanesulfonamide (335)

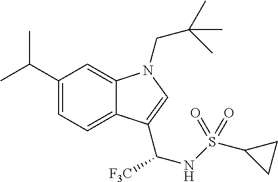 [SMILES: CC(C)c1ccc2c([C@H](NS(=O)(=O)C3CC3)C(F)(F)F)cn(CC(C)(C)C)c2c1]

### Step 1: 6-isopropyl-1-neopentyl-1H-indole

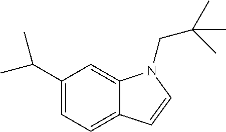 [SMILES: CC(C)c1ccc2ccn(CC(C)(C)C)c2c1]

The title compound was prepared following the same general protocol as described for Example 34, using 6-isopropyl-1H-indole and 1-iodo-2,2-dimethylpropane. ESI-MS (m/z): 229.91 [M+1]⁺.

### Step 2: (S)-2-methyl-N—((S)-2,2,2-trifluoro-1-(6-isopropyl-1-neopentyl-1H-indol-3-yl)ethyl)propane-2-sulfinamide

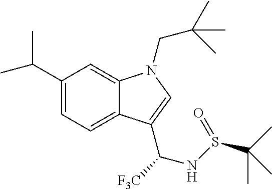 [SMILES: CC(C)c1ccc2c([C@H](N[S@@](=O)C(C)(C)C)C(F)(F)F)cn(CC(C)(C)C)c2c1]

The title compound was prepared following the same general protocol as described for Step 3, Example 116, using (S)-2-methyl-N-(3,3,3-trifluoroprop-1-en-1-yl)propane-2-sulfinamide and 6-isopropyl-1-neopentyl-1H-indole. ESI-MS (m/z): 430.60 [M+1]⁺.

### Step 3: (S)-2,2,2-trifluoro-1-(6-isopropyl-1-neopentyl-1H-indol-3-yl)ethan-1-amine

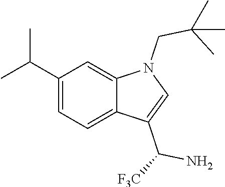 [SMILES: CC(C)c1ccc2c([C@H](N)C(F)(F)F)cn(CC(C)(C)C)c2c1]

The title compound was prepared following the same general protocol as described for Step 4, Example 116, using (S)-2-methyl-N—((S)-2,2,2-trifluoro-1-(6-isopropyl-1-neopentyl-1H-indol-3-yl)ethyl)propane-2-sulfinamide.

### Step 4: (S)—N-(2,2,2-trifluoro-1-(6-isopropyl-1-neopentyl-1H-indol-3-yl)ethyl)cyclopropanesulfonamide

The title compound was prepared following the same general protocol as described for Step 5, Example 116, using (S)-2,2,2-trifluoro-1-(6-isopropyl-1-neopentyl-1H-indol-3-yl)ethan-1-amine. ESI-MS (m/z): 430.86 [M+1]⁺.

### Example 142: (S)—N-(2,2,2-trifluoro-1-(1-isobutyl-6-(trifluoromethyl)-1H-indol-3-yl)ethyl)cyclopropanesulfonamide (344)

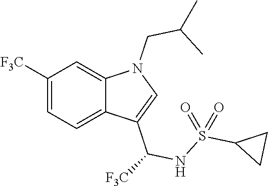 [SMILES: CC(C)Cn1cc([C@H](NS(=O)(=O)C2CC2)C(F)(F)F)c2ccc(C(F)(F)F)cc21]

### Step 1: (S)-2-methyl-N—((S)-2,2,2-trifluoro-1-(1-isobutyl-6-(trifluoromethyl)-1H-indol-3-yl)ethyl)propane-2-sulfinamide

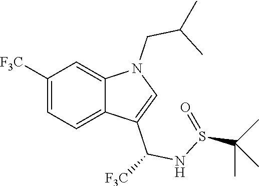 [SMILES: CC(C)Cn1cc([C@H](N[S@@](=O)C(C)(C)C)C(F)(F)F)c2ccc(C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 1-isobutyl-6-(trifluoromethyl)-1H-indole. ESI-MS (m/z): 442.55 [M+1]⁺.

### Step 2: (S)-2,2,2-trifluoro-1-(1-isobutyl-6-(trifluoromethyl)-1H-indol-3-yl)ethan-1-amine

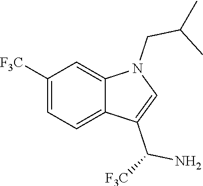 [SMILES: CC(C)Cn1cc([C@H](N)C(F)(F)F)c2ccc(C(F)(F)F)cc21]

The title compound was prepared following the same general protocol as described for Step 4, Example 116, using (S)-2-methyl-N—((S)-2,2,2-trifluoro-1-(1-isobutyl-6-(trifluoromethyl)-1H-indol-3-yl)ethyl)propane-2-sulfinamide.

### Step 3: (S)—N-(2,2,2-trifluoro-1-(1-isobutyl-6-(trifluoromethyl)-1H-indol-3-yl)ethyl)cyclopropanesulfonamide

The title compound was prepared following the same general protocol as described for Step 5, Example 116, using (S)-2,2,2-trifluoro-1-(1-isobutyl-6-(trifluoromethyl)-1H-indol-3-yl)ethan-1-amine. ESI-MS (m/z): 442.82 [M+1]⁺.

### Example 143: (S)—N-(1-(6-chloro-1-isobutyl-1H-indol-3-yl)-2,2,2-trifluoroethyl)cyclopropanesulfonamide (341)

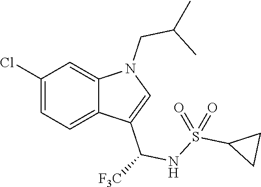 [SMILES: CC(C)Cn1cc([C@H](NS(=O)(=O)C2CC2)C(F)(F)F)c2ccc(Cl)cc21]

### Step 1: (S)—N—((S)-1-(6-chloro-1-isobutyl-1H-indol-3-yl)-2,2,2-trifluoroethyl)-2-methylpropane-2-sulfinamide

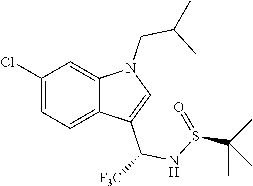 [SMILES: CC(C)Cn1cc([C@H](N[S@@](=O)C(C)(C)C)C(F)(F)F)c2ccc(Cl)cc21]

The title compound was prepared following the same general protocol as described for Step 3, Example 116, using (S)-2-methyl-N-(3,3,3-trifluoroprop-1-en-1-yl)propane-2-sulfinamide and 6-chloro-1-isobutyl-1H-indole. ESI-MS (m/z): 408.54[M+1]⁺.

### Step 2: (S)-1-(6-chloro-1-isobutyl-1H-indol-3-yl)-2,2,2-trifluoroethan-1-amine

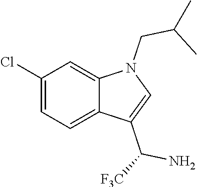 [SMILES: CC(C)Cn1cc([C@H](N)C(F)(F)F)c2ccc(Cl)cc21]

The title compound was prepared following the same general protocol as described for Step 4, Example 116, using (S)—N—((S)-1-(6-chloro-1-isobutyl-1H-indol-3-yl)-2,2,2-trifluoroethyl)-2-methylpropane-2-sulfinamide.

### Step 3: (S)—N-(1-(6-chloro-1-isobutyl-1H-indol-3-yl)-2,2,2-trifluoroethyl)cyclopropanesulfonamide

The title compound was prepared following the same general protocol as described for Step 5, Example 116, using (S)-1-(6-chloro-1-isobutyl-1H-indol-3-yl)-2,2,2-trifluoroethan-1-amine. ESI-MS (m/z): 408.70 [M+1]⁺.

### Example 144: (S)—N-(1-(6-bromo-5-fluoro-1-neopentyl-1H-indol-3-yl)-2,2,2-trifluoroethyl)cyclopropanesulfonamide (342)

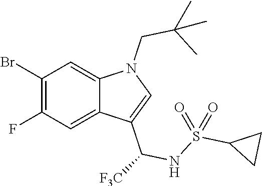 [SMILES: CC(C)(C)Cn1cc([C@H](NS(=O)(=O)C2CC2)C(F)(F)F)c2cc(F)c(Br)cc21]

### Step 1: (S)—N—((S)-1-(6-bromo-5-fluoro-1-neopentyl-1H-indol-3-yl)-2,2,2-trifluoroethyl)-2-methylpropane-2-sulfinamide

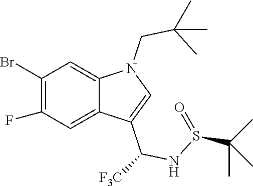 [SMILES: CC(C)(C)Cn1cc([C@H](N[S@@](=O)C(C)(C)C)C(F)(F)F)c2cc(F)c(Br)cc21]

The title compound was prepared following the same general protocol as described for Step 3, Example 116, using (S)-2-methyl-N-(3,3,3-trifluoroprop-1-en-1-yl)propane-2-sulfinamide and 6-bromo-5-fluoro-1-neopentyl-1H-indole. ESI-MS (m/z): 484.55, 486.54 [M+1]⁺.

### Step 2: (S)-1-(6-bromo-5-fluoro-1-neopentyl-1H-indol-3-yl)-2,2,2-trifluoroethan-1-amine

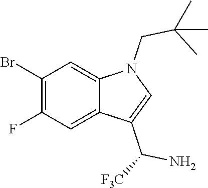 [SMILES: CC(C)(C)Cn1cc([C@H](N)C(F)(F)F)c2cc(F)c(Br)cc21]

The title compound was prepared following the same general protocol as described for Step 4, Example 116, using (S)—N—((S)-1-(6-bromo-5-fluoro-1-neopentyl-1H-indol-3-yl)-2,2,2-trifluoroethyl)-2-methylpropane-2-sulfinamide.

### Step 3: (S)—N-(1-(6-bromo-5-fluoro-1-neopentyl-1H-indol-3-yl)-2,2,2-trifluoroethyl)cyclopropanesulfonamide

The title compound was prepared following the same general protocol as described for Step 5, Example 116, using (S)-1-(6-bromo-5-fluoro-1-neopentyl-1H-indol-3-yl)-2,2,2-trifluoroethan-1-amine. ESI-MS (m/z): 485.74, 486.64 [M+1]⁺.

### Example 145: (S)—N-(1-(6-bromo-1-neopentyl-1H-indol-3-yl)-2,2,2-trifluoroethyl)cyclopropanesulfonamide (343)

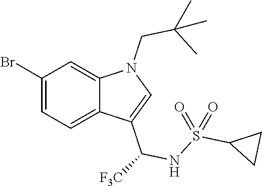 [SMILES: CC(C)(C)Cn1cc([C@H](NS(=O)(=O)C2CC2)C(F)(F)F)c2ccc(Br)cc21]

### Step 1: (S)—N—((S)-1-(6-bromo-1-neopentyl-1H-indol-3-yl)-2,2,2-trifluoroethyl)-2-methylpropane-2-sulfinamide

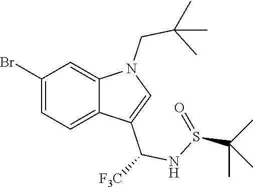 [SMILES: CC(C)(C)Cn1cc([C@H](N[S@@](=O)C(C)(C)C)C(F)(F)F)c2ccc(Br)cc21]

The title compound was prepared following the same general protocol as described for Step 3, Example 116, using (S)-2-methyl-N-(3,3,3-trifluoroprop-1-en-1-yl)propane-2-sulfinamide and 6-bromo-1-neopentyl-1H-indole. ESI-MS (m/z): 466.62, 468.66[M+1]⁺.

### Step 2: (S)-1-(6-bromo-1-neopentyl-1H-indol-3-yl)-2,2,2-trifluoroethan-1-amine

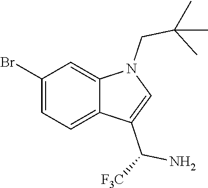 [SMILES: CC(C)(C)Cn1cc([C@H](N)C(F)(F)F)c2ccc(Br)cc21]

The title compound was prepared following the same general protocol as described for Step 4, Example 116, using (S)—N—((S)-1-(6-bromo-1-neopentyl-1H-indol-3-yl)-2,2,2-trifluoroethyl)-2-methylpropane-2-sulfinamide.

### Step 3: (S)—N-(1-(6-bromo-1-neopentyl-1H-indol-3-yl)-2,2,2-trifluoroethyl)cyclopropanesulfonamide

The title compound was prepared following the same general protocol as described for Step 5, Example 116, using (S)-1-(6-bromo-1-neopentyl-1H-indol-3-yl)-2,2,2-trifluoroethan-1-amine. ESI-MS (m/z): 466.62, 468.66 [M+1]⁺.

### Example 146: (S)—N-(2,2,2-trifluoro-1-(6-(4-fluoro-2-(trifluoromethyl)phenyl)-1-isobutyl-1H-indol-3-yl)ethyl)cyclopropanesulfonamide (324)

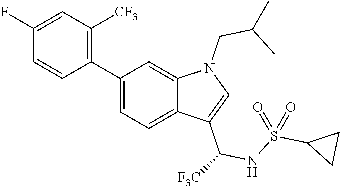 [SMILES: CC(C)Cn1cc([C@H](NS(=O)(=O)C2CC2)C(F)(F)F)c2ccc(-c3ccc(F)cc3C(F)(F)F)cc21]

### Step 1: 6-(4-fluoro-2-(trifluoromethyl)phenyl)-1-isobutyl-1H-indole

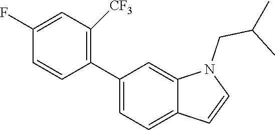 [SMILES: CC(C)Cn1ccc2ccc(-c3ccc(F)cc3C(F)(F)F)cc21]

The title compound was prepared following the same general protocol as described for Step 5, Example 1, using 6-bromo-1-isobutyl-1H-indole and (4-fluoro-2-(trifluoromethyl)phenyl)boronic acid. ESI-MS (m/z): 335.98 [M+1]⁺.

### Step 2: (S)-2-methyl-N—((S)-2,2,2-trifluoro-1-(6-(4-fluoro-2-(trifluoromethyl)phenyl)-1-isobutyl-1H-indol-3-yl)ethyl)propane-2-sulfinamide

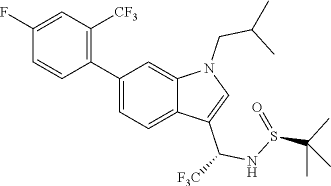 [SMILES: CC(C)Cn1cc([C@H](N[S@@](=O)C(C)(C)C)C(F)(F)F)c2ccc(-c3ccc(F)cc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 6-(4-fluoro-2-(trifluoromethyl)phenyl)-1-isobutyl-1H-indole. ESI-MS (m/z): 536.59 [M+1]⁺.

### Step 3: (S)-2,2,2-trifluoro-1-(6-(4-fluoro-2-(trifluoromethyl)phenyl)-1-isobutyl-1H-indol-3-yl)ethan-1-amine

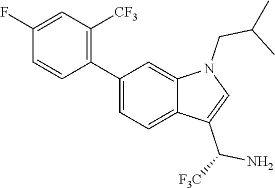 [SMILES: CC(C)Cn1cc([C@H](N)C(F)(F)F)c2ccc(-c3ccc(F)cc3C(F)(F)F)cc21]

The title compound was prepared following the same general protocol as described for Step 4, Example 116, using (S)-2-methyl-N—((S)-2,2,2-trifluoro-1-(6-(4-fluoro-2-(trifluoromethyl)phenyl)-1-isobutyl-1H-indol-3-yl)ethyl)propane-2-sulfinamide.

### Step 4: (S)—N-(2,2,2-trifluoro-1-(6-(4-fluoro-2-(trifluoromethyl)phenyl)-1-isobutyl-1H-indol-3-yl)ethyl)cyclopropanesulfonamide

The title compound was prepared following the same general protocol as described for Step 5, Example 116, using (S)-2,2,2-trifluoro-1-(6-(4-fluoro-2-(trifluoromethyl)phenyl)-1-isobutyl-1H-indol-3-yl)ethan-1-amine. ESI-MS (m/z): 536.71 [M+1]⁺. ¹HNMR (CDCl₃, 400 MHz) δ 7.75 (d, J=8.0 Hz, 1H), 7.48 (dd, J=8.0 Hz, 4.0 Hz, 1H), 7.39-7.36 (m, 1H), 7.29-7.24 (m, 3H), 7.12 (d, J=8.0 Hz, 1H), 5.43 (q, J=8.0 Hz, 1H), 4.97 (d, J=8.0 Hz, 1H), 3.89 (d, J=8.0 Hz, 2H), 2.46-2.41 (m, 1H), 2.21-2.04 (m, 1H), 1.41-1.10 (m, 2H), 0.93-0.89 (m, 8H)

### Example 147: dimethyl({[(1S)-2,2,2-trifluoro-1-[5-fluoro-1-(2-methylpropyl)-6-[2-(trifluoromethyl)phenyl]-1H-indol-3-yl]ethyl]sulfamoyl})amine (320)

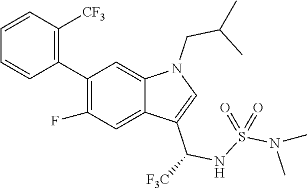 [SMILES: CC(C)Cn1cc([C@H](NS(=O)(=O)N(C)C)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-isobutyl-6-(2-(trifluoromethyl)phenyl)-1H-indol-3-yl)ethan-1-amine and dimethylsulfamoyl chloride. ESI-MS (m/z): 539.40 [M+1]⁺.

### Example 148: dimethyl({[(1S)-2,2,2-trifluoro-1-[5-fluoro-6-methyl-1-(2-methylpropyl)-1H-indol-3-yl]ethyl]sulfamoyl})amine (318)

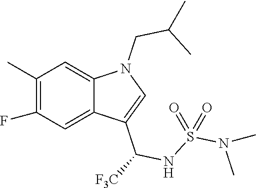 [SMILES: Cc1cc2c(cc1F)c([C@H](NS(=O)(=O)N(C)C)C(F)(F)F)cn2CC(C)C]

The title compound was prepared following the same general protocol as described for Step 5, Example 116, using (S)-2,2,2-trifluoro-1-(5-fluoro-1-isobutyl-6-methyl-1H-indol-3-yl)ethan-1-amine and dimethylsulfamoyl chloride. ESI-MS (m/z): 409.46 [M+1]⁺. ¹HNMR (CDCl₃, 400 MHz) δ 7.32 (d, J=12 Hz, 1H), 7.14 (s, 1H), 7.09 (d, J=8.0 Hz, 1H), 5.11 (q, J=8.0 Hz, 1H), 4.73 (d, J=8.0 Hz, 1H), 3.84 (d, J=8.0 Hz, 2H), 2.75 (s, 6H), 2.40 (s, 3H), 2.23-2.11 (m, 1H) 0.91 (d, J=8.0 Hz, 6H)

### Example 149: {[(1S)-1-[6-(2-chlorophenyl)-5-fluoro-1-(2-methylpropyl)-1H-indol-3-yl]-2,2,2-trifluoroethyl]sulfamoyl}dimethylamine (326)

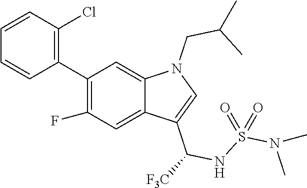 [SMILES: CC(C)Cn1cc([C@H](NS(=O)(=O)N(C)C)C(F)(F)F)c2cc(F)c(-c3ccccc3Cl)cc21]

The title compound was prepared following the same general protocol as described for Step 5, Example 116, using (S)-1-(6-(2-chlorophenyl)-5-fluoro-1-isobutyl-1H-indol-3-yl)-2,2,2-trifluoroethan-1-amine and dimethylsulfamoyl chloride. ESI-MS (m/z): 505.44 [M+1]⁺. ¹HNMR (CDCl₃, 400 MHz) δ 7.57-7.38 (m, 2H), 7.35-7.31 (m, 3H), 7.27-7.24 (m, 2H), 5.19 (q, J=8.0 Hz, 1H), 4.77 (d, J=8.0 Hz, 1H), 3.89 (d, J=4.0 Hz, 2H), 2.80 (s, 6H), 2.22-2.12 (m, 1H), 0.92 (d, J=8.0 Hz, 6H)

### Example 150: {[(1S)-1-[6-cyclopropyl-5-fluoro-1-(2-methylpropyl)-1H-indol-3-yl]-2,2,2-trifluoroethyl]sulfamoyl}dimethylamine (329)

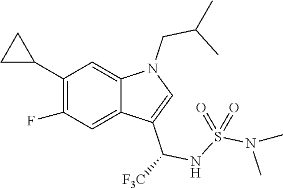 [SMILES: CC(C)Cn1cc([C@H](NS(=O)(=O)N(C)C)C(F)(F)F)c2cc(F)c(C3CC3)cc21]

The title compound was prepared following the same general protocol as described for Step 5, Example 116, using (S)-1-(6-cyclopropyl-5-fluoro-1-isobutyl-1H-indol-3-yl)-2,2,2-trifluoroethan-1-amine and dimethylsulfamoyl chloride. ESI-MS (m/z): 435.46 [M+1]⁺. ¹HNMR (CDCl₃, 400 MHz) δ 7.32 (d, J=12.0 Hz, 1H), 7.14 (s, 1H), 6.85 (d, J=8.0 Hz, 1H), 5.12 (q, J=8.0 Hz, 1H), 4.70 (d, J=8.0 Hz, 1H), 3.83 (d, J=8.0 Hz, 2H), 2.75 (s, 6H), 2.19-1.95 (m, 2H), 1.03-0.98 (m, 2H), 0.91 (d, J=8.0 Hz, 6H), 0.74-0.70 (m, 2H)

### Example 151: {[(1S)-1-[1-(2,2-dimethylpropyl)-5-fluoro-6-methyl-1H-indol-3-yl]-2,2,2-trifluoroethyl]sulfamoyl}dimethylamine (317)

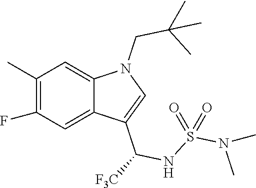 [SMILES: Cc1cc2c(cc1F)c([C@H](NS(=O)(=O)N(C)C)C(F)(F)F)cn2CC(C)(C)C]

The title compound was prepared following the same general protocol as described for Step 5, Example 116, using (S)-2,2,2-trifluoro-1-(5-fluoro-6-methyl-1-neopentyl-1H-indol-3-yl)ethan-1-amine and dimethylsulfamoyl chloride. ESI-MS (m/z): 423.48 [M+1]⁺. ¹HNMR (CDCl₃, 400 MHz) δ 7.30 (d, J=8.0 Hz, 1H), 7.13 (s, 1H), 7.11 (d, J=8.0 Hz, 1H), 5.12 (q, J=8.0 Hz, 1H), 4.74 (d, J=8.0 Hz, 1H), 3.84 (s, 2H), 2.75 (s, 6H), 2.40 (d, J=4.0 Hz, 3H), 0.98 (s, 9H)

### Example 152: {[(1S)-1-[1-(2,2-dimethylpropyl)-5-fluoro-6-[2-(trifluoromethyl)phenyl]-1H-indol-3-yl]-2,2,2-trifluoroethyl]sulfamoyl}dimethylamine (321)

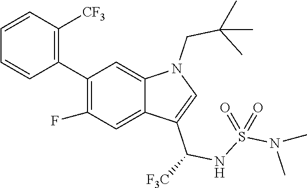 [SMILES: CN(C)S(=O)(=O)N[C@@H](c1cn(CC(C)(C)C)c2cc(-c3ccccc3C(F)(F)F)c(F)cc12)C(F)(F)F]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and dimethylsulfamoyl chloride. ESI-MS (m/z): 553.54 [M+1]⁺.

### Example 153: {[(1S)-1-[6-(2-chlorophenyl)-1-(2,2-dimethylpropyl)-5-fluoro-1H-indol-3-yl]-2,2,2-trifluoroethyl]sulfamoyl}dimethylamine (330)

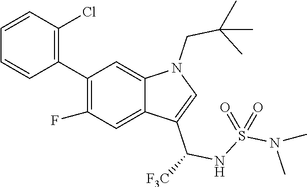 [SMILES: CN(C)S(=O)(=O)N[C@@H](c1cn(CC(C)(C)C)c2cc(-c3ccccc3Cl)c(F)cc12)C(F)(F)F]

The title compound was prepared following the same general protocol as described for Step 5, Example 116, using (S)-1-(6-(2-chlorophenyl)-5-fluoro-1-neopentyl-1H-indol-3-yl)-2,2,2-trifluoroethan-1-amine and dimethylsulfamoyl chloride. ESI-MS (m/z): 520.37 [M+1]⁺. ¹HNMR (CDCl₃, 400 MHz) δ 7.51-7.43 (m, 2H), 7.36-7.30 (m, 3H), 7.27-7.23 (m, 2H), 5.17 (q, J=8.0 Hz, 1H), 4.76 (d, J=8.0 Hz, 1H), 3.86 (s, 2H), 2.78 (s, 6H), 0.97 (s, 9H)

### Example 154: {[(1S)-1-[6-cyclopropyl-1-(2,2-dimethylpropyl)-5-fluoro-1H-indol-3-yl]-2,2,2-trifluoroethyl]sulfamoyl}dimethylamine (328)

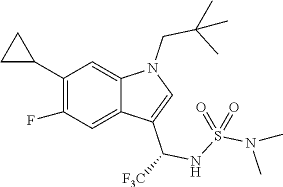 [SMILES: CN(C)S(=O)(=O)N[C@@H](c1cn(CC(C)(C)C)c2cc(C3CC3)c(F)cc12)C(F)(F)F]

The title compound was prepared following the same general protocol as described for Step 5, Example 116, using (S)-1-(6-cyclopropyl-5-fluoro-1-neopentyl-1H-indol-3-yl)-2,2,2-trifluoroethan-1-amine and dimethylsulfamoyl chloride. ESI-MS (m/z): 449.49 [M+1]⁺. ¹HNMR (CDCl₃, 400 MHz) δ 7.30 (d, J=12.0 Hz, 1H), 7.12 (s, 1H), 6.87 (d, J=8.0 Hz, 1H), 5.12 (q, J=8.0 Hz, 1H), 4.72 (d, J=8.0 Hz, 1H), 3.82 (s, 2H), 2.76 (s, 6H), 2.19-2.12 (m, 1H), 1.25-0.99 (m, 11H), 0.71-0.67 (m, 2H)

### Example 155: {[(1S)-1-[1-(2,2-dimethylpropyl)-6-[2-(trifluoromethyl)phenyl]-1H-indol-3-yl]-2,2,2-trifluoroethyl]sulfamoyl}dimethylamine (323)

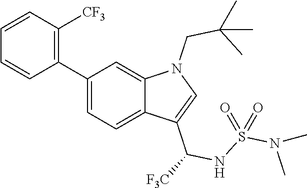 [SMILES: CN(C)S(=O)(=O)N[C@@H](c1cn(CC(C)(C)C)c2cc(-c3ccccc3C(F)(F)F)ccc12)C(F)(F)F]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and dimethylsulfamoyl chloride. ESI-MS (m/z): 535.48 [M+1]⁺. ¹HNMR (CDCl₃, 400 MHz) δ 7.76 (d, J=8.0 Hz, 1H), 7.73 (d, J=8.0 Hz, 1H), 7.56 (t, J=8.0 Hz, 1H), 7.47 (t, J=8.0 Hz, 1H), 7.36 (d, J=8.0 Hz, 1H), 7.32 (s, 1H), 7.22 (s, 1H), 7.13 (d, J=8.0 Hz, 1H), 5.26 (q, J=8.0 Hz, 1H), 4.78 (d, J=8.0 Hz, 1H), 3.89 (s, 2H), 2.76 (s, 6H), 0.99 (s, 9H)

### Example 156: {[(1S)-1-[6-(2-chlorophenyl)-1-(2,2-dimethylpropyl)-1H-indol-3-yl]-2,2,2-trifluoroethyl]sulfamoyl}dimethylamine (338)

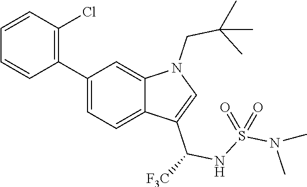 [SMILES: CN(C)S(=O)(=O)N[C@@H](c1cn(CC(C)(C)C)c2cc(-c3ccccc3Cl)ccc12)C(F)(F)F]

The title compound was prepared following the same general protocol as described for Step 5, Example 116, using (S)-1-(6-(2-chlorophenyl)-1-neopentyl-1H-indol-3-yl)-2,2,2-trifluoroethan-1-amine and dimethylsulfamoyl chloride. ESI-MS (m/z): 501.45 [M+1]⁺. ¹HNMR (CDCl₃, 400 MHz) δ 7.57 (d, J=12.0 Hz, 1H), 7.30 (d, J=12.0 Hz, 1H), 7.19 (d, J=8.0 Hz, 1H), 7.15-7.09 (m, 2H), 7.06-7.03 (m, 3H), 5.07 (q, J=8.0 Hz, 1H), 4.57 (d, J=8.0 Hz, 1H), 3.71 (s, 2H), 2.60 (s, 6H), 0.81 (s, 9H)

### Example 157: {[(1S)-1-[1-(2,2-dimethylpropyl)-5-fluoro-6-(propan-2-yl)-1H-indol-3-yl]-2,2,2-trifluoroethyl]sulfamoyl}dimethylamine (337)

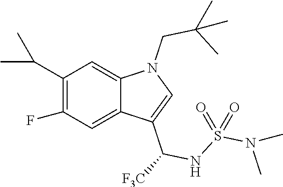 [SMILES: CC(C)c1cc2c(cc1F)c([C@H](NS(=O)(=O)N(C)C)C(F)(F)F)cn2CC(C)(C)C]

The title compound was prepared following the same general protocol as described for Step 5, Example 116, using (S)-2,2,2-trifluoro-1-(5-fluoro-6-isopropyl-1-neopentyl-1H-indol-3-yl)ethan-1-amine and dimethylsulfamoyl chloride. ESI-MS (m/z): 452.14 [M+1]⁺.

### Example 158: dimethyl({[(1S)-2,2,2-trifluoro-1-[1-(2-methylpropyl)-6-(propan-2-yl)-1H-indol-3-yl]ethyl]sulfamoyl})amine (339)

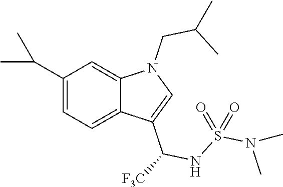 [SMILES: CC(C)Cn1cc([C@H](NS(=O)(=O)N(C)C)C(F)(F)F)c2ccc(C(C)C)cc21]

The title compound was prepared following the same general protocol as described for Step 5, Example 116, using (S)-2,2,2-trifluoro-1-(1-isobutyl-6-isopropyl-1H-indol-3-yl)ethan-1-amine and dimethylsulfamoyl chloride. ESI-MS (m/z): 419.63 [M+1]⁺.

### Example 159: {[(1S)-1-[1-(2,2-dimethylpropyl)-6-(propan-2-yl)-1H-indol-3-yl]-2,2,2-trifluoroethyl]sulfamoyl}dimethylamine (340)

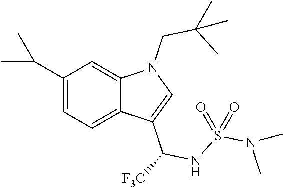 [SMILES: CC(C)c1ccc2c([C@H](NS(=O)(=O)N(C)C)C(F)(F)F)cn(CC(C)(C)C)c2c1]

The title compound was prepared following the same general protocol as described for Step 5, Example 116, using (S)-2,2,2-trifluoro-1-(6-isopropyl-1-neopentyl-1H-indol-3-yl)ethan-1-amine and dimethylsulfamoyl chloride. ESI-MS (m/z): 433.57 [M+1]⁺.

General Procedures for the Suzuki Coupling

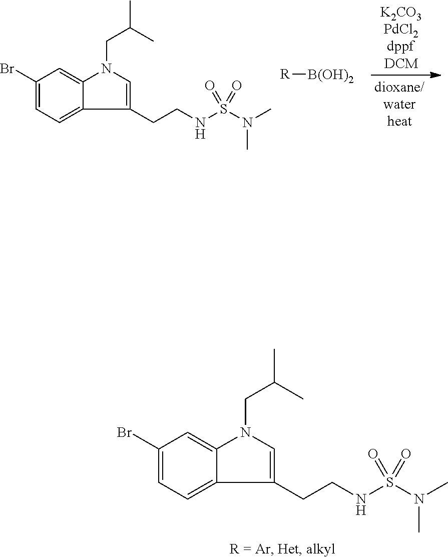

To a mixture of N′-{2-[6-bromo-1-(2-methylpropyl)-1H-indol-3-yl]ethyl}-N,N-dimethyl-sulfuric diamide 30 mg (0.08 mmol), boronic acid (0.12 mmol) and potassium carbonate 22 mg (0.16 mmol) in 1 mL of dioxane and water (v/v=1:1) was added 3 mg of Pd catalyst. The mixture was heated at 100° C. and monitored by LCMS. The solid was filtered and the filtrate was purified by prep-HPLC to obtain the desired product.

### Example 160: N-{2-[6-(4-fluorophenyl)-1-(2-methylpropyl)-1H-indol-3-yl]ethyl}-N,N-dimethylsulfuric diamide (45)

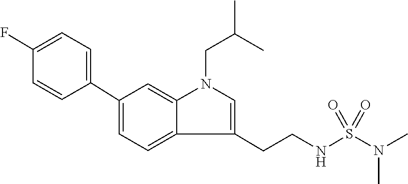 [SMILES: CC(C)Cn1cc(CCNS(=O)(=O)N(C)C)c2ccc(-c3ccc(F)cc3)cc21]

The compound was prepared following the general procedure of Suzuki coupling. ESI-MS (m/z): 417.91 [M+1]⁺.

### Example 161: N-{2-[6-(2-methylphenyl)-1-(2-methylpropyl)-1H-indol-3-yl]ethyl}-N,N-dimethylsulfuric diamide (46)

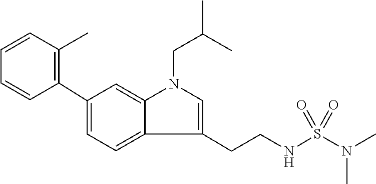 [SMILES: Cc1ccccc1-c1ccc2c(CCNS(=O)(=O)N(C)C)cn(CC(C)C)c2c1]

The compound was prepared following the general procedure of Suzuki coupling. ESI-MS (m/z): 413.91 [M+1]⁺.

### Example 162: N-{2-[6-(3-methylphenyl)-1-(2-methylpropyl)-1H-indol-3-yl]ethyl}-N,N-dimethylsulfuric diamide (47)

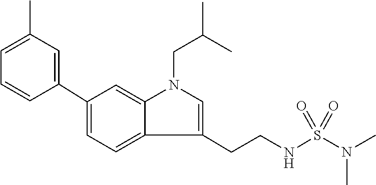 [SMILES: Cc1cccc(-c2ccc3c(CCNS(=O)(=O)N(C)C)cn(CC(C)C)c3c2)c1]

The compound was prepared following the general procedure of Suzuki coupling. ESI-MS (m/z): 413.91 [M+1]⁺.

### Example 163: N-{2-[6-(2-methoxylphenyl)-1-(2-methylpropyl)-1H-indol-3-yl]ethyl}-N,N-dimethylsulfuric diamide (58)

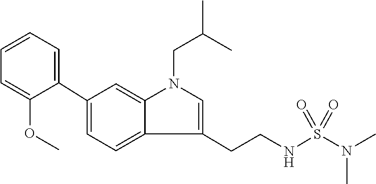 [SMILES: COc1ccccc1-c1ccc2c(CCNS(=O)(=O)N(C)C)cn(CC(C)C)c2c1]

The compound was prepared following the general procedure of Suzuki coupling. ESI-MS (m/z): 429.98 [M+1]⁺.

### Example 164: N-{2-[6-(3-cyanophenyl)-1-(2-methylpropyl)-1H-indol-3-yl]ethyl}-N,N-dimethylsulfuric diamide (59)

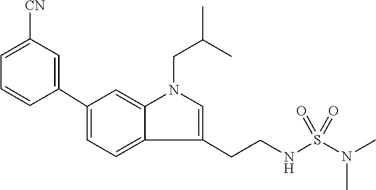 [SMILES: CC(C)Cn1cc(CCNS(=O)(=O)N(C)C)c2ccc(-c3cccc(C#N)c3)cc21]

The compound was prepared following the general procedure of Suzuki coupling. ESI-MS (m/z): 424.87 [M+1]⁺.

### Example 165: N-{2-[6-(3-methylsulfonylphenyl)-1-(2-methylpropyl)-1H-indol-3-yl]ethyl}-N,N-dimethylsulfuric diamide (60)

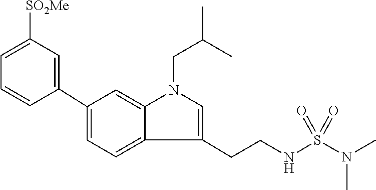 [SMILES: CC(C)Cn1cc(CCNS(=O)(=O)N(C)C)c2ccc(-c3cccc(S(C)(=O)=O)c3)cc21]

The compound was prepared following the general procedure of Suzuki coupling. ESI-MS (m/z): 477.86 [M+1]⁺.

### Example 166: N-{2-[6-(4-methylphenyl)-1-(2-methylpropyl)-1H-indol-3-yl]ethyl}-N,N-dimethylsulfuric diamide (48)

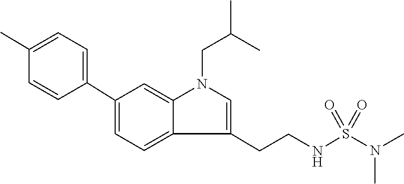

The compound was prepared following the general procedure of Suzuki coupling. ESI-MS (m/z): 413.91 [M+1]⁺.

### Example 167: N-{2-[6-(3-nitrophenyl)-1-(2-methylpropyl)-1H-indol-3-yl]ethyl}-N,N-dimethylsulfuric diamide (49)

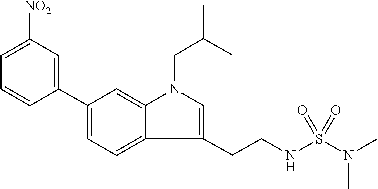 [SMILES: CC(C)Cn1cc(CCNS(=O)(=O)N(C)C)c2ccc(-c3cccc([N+](=O)[O-])c3)cc21]

The compound was prepared following the general procedure of Suzuki coupling, M+1 calculated for C₂₂H₂₉N₄O₄S 445.56, found 445.93.

### Example 168: N-{2-[6-(3-trifluoromethylphenyl)-1-(2-methylpropyl)-1H-indol-3-yl]ethyl}-N,N-dimethyl-sulfuric diamide (50)

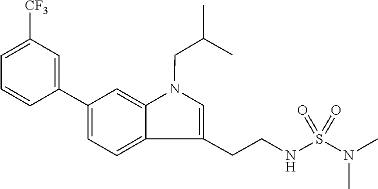 [SMILES: CC(C)Cn1cc(CCNS(=O)(=O)N(C)C)c2ccc(-c3cccc(C(F)(F)F)c3)cc21]

The compound was prepared following the general procedure of Suzuki coupling, M+1 calculated for C₂₃H₂₉F₃N₃O₂S 468.56, found 468.97.

### Example 169: N-{2-[6-(4-trifluoromethoxylphenyl)-1-(2-methylpropyl)-1H-indol-3-yl]ethyl}-N,N-dimethylsulfuric diamide (51)

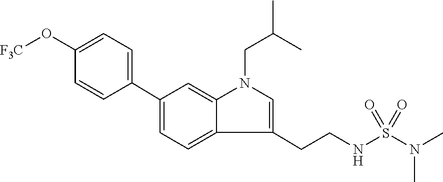 [SMILES: CC(C)Cn1cc(CCNS(=O)(=O)N(C)C)c2ccc(-c3ccc(OC(F)(F)F)cc3)cc21]

The compound was prepared following the general procedure of Suzuki coupling, M+1 calculated for C₂₃H₂₉F₃N₃O₃S 484.19, found 484.91.

### Example 170: N-{2-[6-(4-trifluoromethylphenyl)-1-(2-methylpropyl)-1H-indol-3-yl]ethyl}-N,N-dimethyl-sulfuric diamide (52)

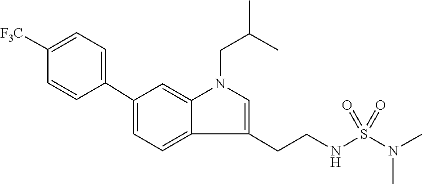 [SMILES: CC(C)Cn1cc(CCNS(=O)(=O)N(C)C)c2ccc(-c3ccc(C(F)(F)F)cc3)cc21]

The compound was prepared following the general procedure of Suzuki coupling, M+1 calculated for C₂₃H₂₉F₃N₃O₂S 468.19, found 468.91.

### Example 171: N-{2-[6-(3-methoxylphenyl)-1-(2-methylpropyl)-1H-indol-3-yl]ethyl}-N,N-dimethyl-sulfuric diamide (53)

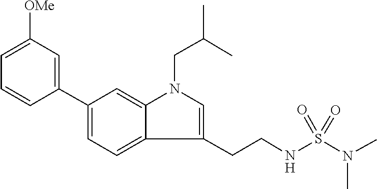 [SMILES: COc1cccc(-c2ccc3c(CCNS(=O)(=O)N(C)C)cn(CC(C)C)c3c2)c1]

The compound was prepared following the general procedure of Suzuki coupling, M+1 calculated for C₂₃H₃₂N₃O₃S: 430.22 found: 431.00.

### Example 172: N-{2-[6-(3,5-difluorophenyl)-1-(2-methylpropyl)-1H-indol-3-yl]ethyl}-N,N-dimethyl-sulfuric diamide (54)

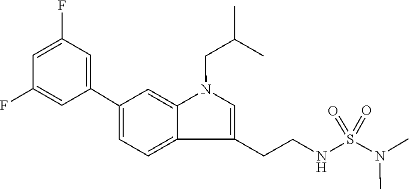 [SMILES: CC(C)Cn1cc(CCNS(=O)(=O)N(C)C)c2ccc(-c3cc(F)cc(F)c3)cc21]

The compound was prepared following the general procedure of Suzuki coupling, M+1 calculated for C₂₂H₂₈F₂N₃O₂S 436.19, found: 436.91.

### Example 173: N-{2-[6-([1,1′-biphenyl]-3-yl)-1-(2-methylpropyl)-1H-indol-3-yl]ethyl}-N,N-dimethyl-sulfuric diamide (55)

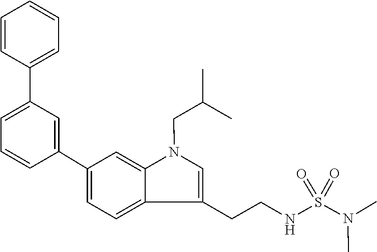 [SMILES: CC(C)Cn1cc(CCNS(=O)(=O)N(C)C)c2ccc(-c3cccc(-c4ccccc4)c3)cc21]

The compound was prepared following the general procedure of Suzuki coupling, M+1 calculated for C₂₈H₃₄N₃O₂S 476.24, found 476.92.

### Example 174: N-{2-[6-(2-fluorophenyl)-1-(2-methylpropyl)-1H-indol-3-yl]ethyl}-N,N-dimethylsulfuric diamide (56)

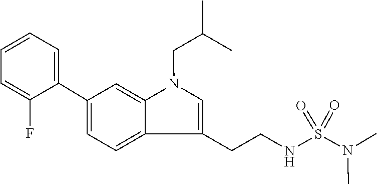 [SMILES: CC(C)Cn1cc(CCNS(=O)(=O)N(C)C)c2ccc(-c3ccccc3F)cc21]

The compound was prepared following the general procedure of Suzuki coupling, M+1 calculated for C₂₂H₂₉FN₃O₂S 418.20, found 417.94.

### Example 175: N-{2-[6-(3-fluorophenyl)-1-(2-methylpropyl)-1H-indol-3-yl]ethyl}-N,N-dimethylsulfuric diamide (57)

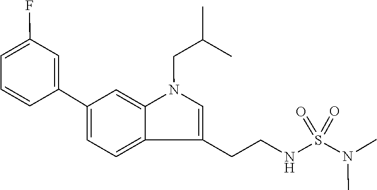 [SMILES: CC(C)Cn1cc(CCNS(=O)(=O)N(C)C)c2ccc(-c3cccc(F)c3)cc21]

The compound was prepared following the general procedure of Suzuki coupling, M+1 calculated for C₂₂H₂₉FN₃O₂S 418.20, found 417.88.

### Example 176: N′-{2-[6-(2-N′-methylsulfonamido phenyl)-1-(2-methylpropyl)-1H-indol-3-yl]ethyl}-N,N-dimethylsulfuric diamide (72)

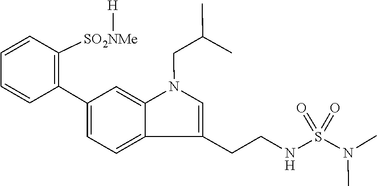

The compound was prepared following the general procedure of Suzuki coupling, M+1 calculated for C₂₃H₃₁N₄O₄S₂ 493.19, found 493.09.

### Example 177: N′-{2-[6-(2-methyl carbonate phenyl)-1-(2-methylpropyl)-1H-indol-3-yl]ethyl}-N,N-dimethyl sulfuric diamide (73)

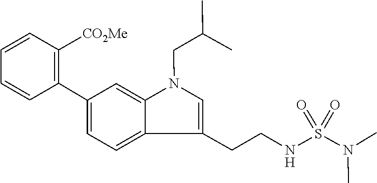 [SMILES: COC(=O)c1ccccc1-c1ccc2c(CCNS(=O)(=O)N(C)C)cn(CC(C)C)c2c1]

The compound was prepared following the general procedure of Suzuki coupling, M+1 calculated for C₂₄H₃₂N₃O₄S 458.21, found 457.95.

### Example 178: N-{2-[6-[(1,1-diphenyl)-2yl]-1-(2-methylpropyl)-1H-indol-3-yl]ethyl}-N,N-dimethylsulfuric diamide (74)

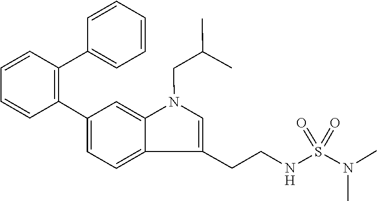 [SMILES: CC(C)Cn1cc(CCNS(=O)(=O)N(C)C)c2ccc(-c3ccccc3-c3ccccc3)cc21]

The compound was prepared following the general procedure of Suzuki coupling, M+1 calculated for C₂₈H₃₄N₃O₂S 476.24, found 475.49.

### Example 179: N-{2-[6-(2-hydroxylphenyl)-1-(2-methylpropyl)-1H-indol-3-yl]ethyl}-N,N-dimethylsulfuric diamide (75)

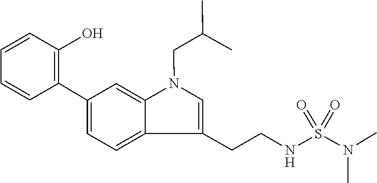 [SMILES: CC(C)Cn1cc(CCNS(=O)(=O)N(C)C)c2ccc(-c3ccccc3O)cc21]

The compound was prepared following the general procedure of Suzuki coupling, M+1 calculated for C₂₂H₃₀N₃O₃S 416.20, found 415.89.

### Example 180: N-{2-[6-(2-hydroxylmethylphenyl)-1-(2-methylpropyl)-1H-indol-3-yl]ethyl}-N,N-dimethyl-sulfuric diamide (76)

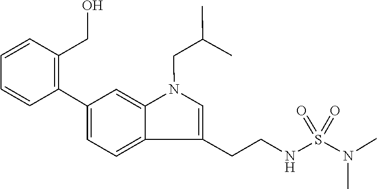 [SMILES: CC(C)Cn1cc(CCNS(=O)(=O)N(C)C)c2ccc(-c3ccccc3CO)cc21]

The compound was prepared following the general procedure of Suzuki coupling, M+1 calculated for C₂₃H₃₂N₃O₃S 430.22, found 429.74.

### Example 181: N-{2-[6-(2-benzyloxylphenyl)-1-(2-methylpropyl)-1H-indol-3-yl]ethyl}-N,N-dimethyl-sulfuric diamide (77)

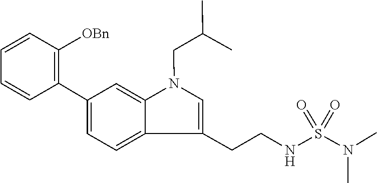 [SMILES: CC(C)Cn1cc(CCNS(=O)(=O)N(C)C)c2ccc(-c3ccccc3OCc3ccccc3)cc21]

The compound was prepared following the general procedure of Suzuki coupling, M+1 calculated for C₂₉H₃₆N₃O₂S 506.25, found 505.94.

### Example 182: N-{2-[6-(2-chlorophenyl)-1-(2-methylpropyl)-1H-indol-3-yl]ethyl}-N,N-dimethylsulfuric diamide (78)

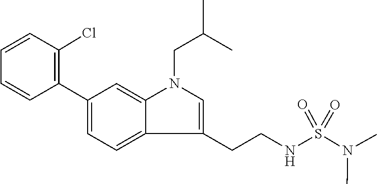 [SMILES: CC(C)Cn1cc(CCNS(=O)(=O)N(C)C)c2ccc(-c3ccccc3Cl)cc21]

The compound was prepared following the general procedure of Suzuki coupling, M+1 calculated for C₂₂H₂₉ClN₃O₂S 434.17, found 433.83.

### Example 183: N-{2-[6-(4-tert-butyl carbonate phenyl)-1-(2-methylpropyl)-1H-indol-3-yl]ethyl}-N,N-dimethylsulfuric diamide (79)

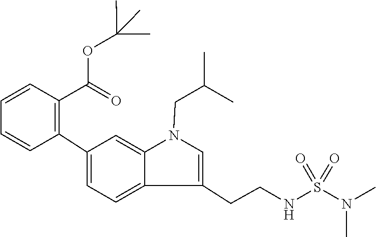 [SMILES: CC(C)Cn1cc(CCNS(=O)(=O)N(C)C)c2ccc(-c3ccccc3C(=O)OC(C)(C)C)cc21]

The compound was prepared following the general procedure of Suzuki coupling, M+1 calculated for C₂₇H₃₈N₃O₄S 500.26, found 499.56.

### Example 184: N-{2-[6-(2,6-dichlorophenyl)-1-(2-methylpropyl)-1H-indol-3-yl]ethyl}-N,N-dimethyl-sulfuric diamide (80)

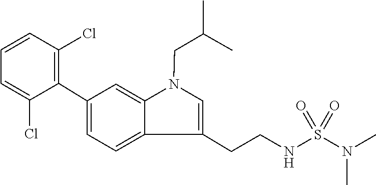 [SMILES: CC(C)Cn1cc(CCNS(=O)(=O)N(C)C)c2ccc(-c3c(Cl)cccc3Cl)cc21]

The compound was prepared following the general procedure of Suzuki coupling, M+1 calculated for C₂₂H₂₈Cl₂N₃O₂S 468.13, found 467.85.

### Example 185: N-{2-[6-(2,6-dimethylphenyl)-1-(2-methylpropyl)-1H-indol-3-yl]ethyl}-N,N-dimethyl-sulfuric diamide (81)

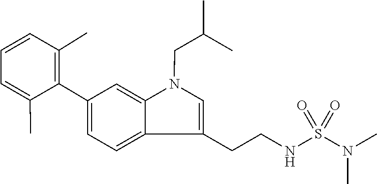 [SMILES: Cc1cccc(C)c1-c1ccc2c(CCNS(=O)(=O)N(C)C)cn(CC(C)C)c2c1]

The compound was prepared following the general procedure of Suzuki coupling, M+1 calculated for C₂₄H₃₄N₃O₂S 428.24, found 427.93.

### Example 186: N-{2-[6-cyclopropyl-1-(2-methylpropyl)-1H-indol-3-yl]ethyl}-N,N-dimethylsulfuric diamide (82)

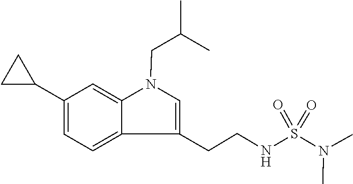 [SMILES: CC(C)Cn1cc(CCNS(=O)(=O)N(C)C)c2ccc(C3CC3)cc21]

The compound was prepared following the general procedure of Suzuki coupling, M+1 calculated for C₁₉H₃₀N₃O₂S 364.21, found 363.96.

General Procedures for Ullmann Reaction

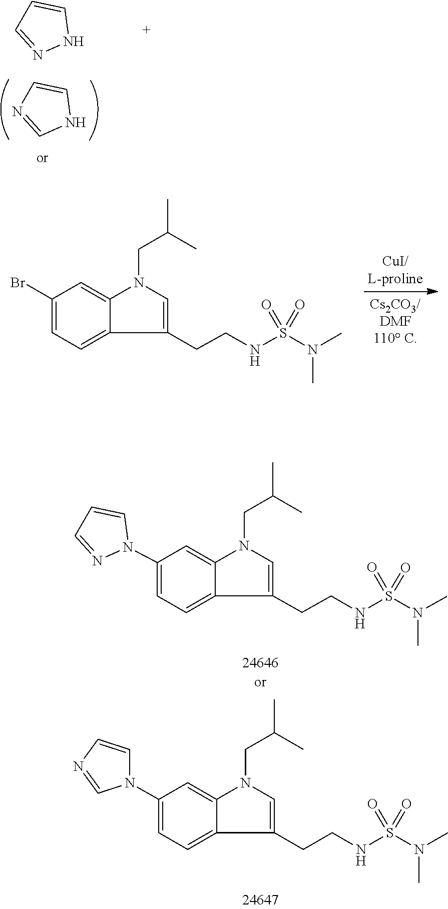

Charge N′-{2-[6-bromo-1-(2-methylpropyl)-1H-indol-3-yl]ethyl}-N,N-dimethylsulfuric diamide 16 mg (0.04 mmol, 1.0 equiv.), 1H-pyrazole (or 1H-imidazole) 8 mg (0.12 mmol, 3.0 equiv.), L-proline 1.8 mg (0.04 equiv.), Cs₂CO₃ 26 mg (0.08 mmol, 2.0 equiv.) and 0.8 mL of DMF into the vial, the mixture was degassed three times. Then CuI 1.5 mg (0.2 equiv.) was added and the reaction mixture was degassed three times again followed by heating in a 110° C. oil bath. The reaction mixture was monitored by LCMS and purified by prep-HPLC to obtain the corresponding product.

### Example 187: N-{2-[6-(1H-pyrazol-1-yl)-1-(2-methylpropyl)-1H-indol-3-yl]ethyl}-N,N-dimethylsulfuric diamide (92)

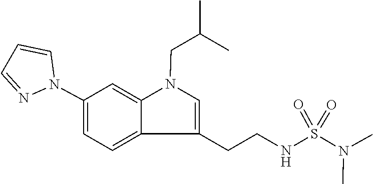 [SMILES: CC(C)Cn1cc(CCNS(=O)(=O)N(C)C)c2ccc(-n3cccn3)cc21]

The compound was prepared following the general procedure from 1H-pyrazole, M+1 calculated for C₁₉H₂₈N₅O₂S 390.20, found 389.97.

### Example 188: N-{2-[6-(1H-imidazol-1-yl)-1-(2-methylpropyl)-1H-indol-3-yl]ethyl}-N,N-dimethylsulfuric diamide (93)

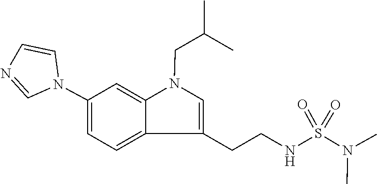 [SMILES: CC(C)Cn1cc(CCNS(=O)(=O)N(C)C)c2ccc(-n3ccnc3)cc21]

The compound was prepared following the general procedure from 1H-imidazole, M+1 calculated for C₁₉H₂₈N₅O₂S 390.20, found 389.96.

General Procedure for the Reduction Amination

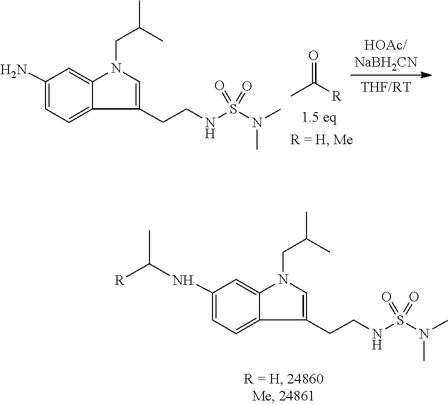

Charge N′-{2-[6-amino-1-(2-methylpropyl)-1H-indol-3-yl]ethyl}-N,N-dimethylsulfuric diamide 22 mg (0.06 mmol, 1.0 equiv.) and acetone (or acetyl aldehyde) 2 equiv. and anhydrous THF 1.0 mL into the vial, then acetic acid 10 uL was added and the reaction mixture was cooled in ice-water bath and then NaBH₃CN (3.0 equiv.) was added, and the reaction mixture was stirred at room temperature. After the reaction completed, the product was purified by prep-HPLC to obtain the desired product.

### Example 189: N-{2-[6-isopropylamino-1-(2-methylpropyl)-1H-indol-3-yl]ethyl}-N,N-dimethylsulfuric diamide (111)

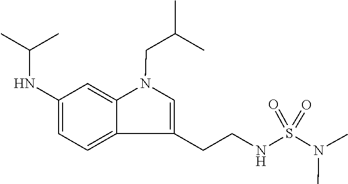 [SMILES: CC(C)Cn1cc(CCNS(=O)(=O)N(C)C)c2ccc(NC(C)C)cc21]

M+1 calculated for C₁₉H₃₃N₄O₂S 381.23, found 380.96.

### Example 190: N′-{2-[6-ethylamino-1-(2-methylpropyl)-1H-indol-3-yl]ethyl}-N,N-dimethylsulfuric diamide (112)

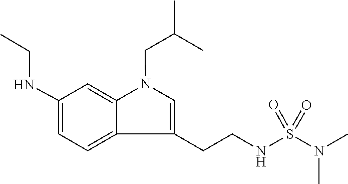 [SMILES: CCNc1ccc2c(CCNS(=O)(=O)N(C)C)cn(CC(C)C)c2c1]

M+1 calculated for C₁₈H₃₁N₄O₂S 367.22, found 366.99.

### Example 191: N′-{2-[6-N,N-dimethylamino-1-(2-methylpropyl)-1H-indol-3-yl]ethyl}-N,N-dimethylsulfuric diamide (113)

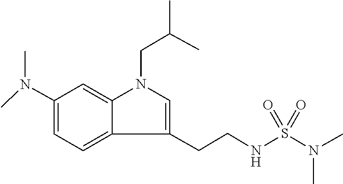

N′-{2-[6-amino-1-(2-methylpropyl)-1H-indol-3-yl]ethyl}-N,N-dimethylsulfuric diamide 20 mg (0.05 mmol, 1.0 equiv.) and paraformaldehyde 18 mg (0.25 mmol, 5.0 equiv.) and HOAc 0.5 mL were stirred at room temperature for 30 mins. NaBH₃CN 5 mg was added to the vial at 0° C., the reaction mixture was stirred at room temperature and monitored by LCMS. After the reaction completed, the reaction mixture was purified by prep-HPLC. M+1 calculated for C₁₈H₃₁N₄O₂S 367.22, found 367.03.

### Example 192: N′-{2-[6-N,N-dimethylamino-1-(2-methylpropyl)-1H-indol-3-yl]ethyl}-N′-methyl-N,N-dimethyl sulfuric diamide (114)

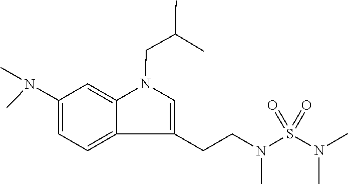

N′-{2-[6-amino-1-(2-methylpropyl)-1H-indol-3-yl]ethyl}-N,N-dimethylsulfuric diamide 20 mg (0.05 mmol, 1.0 equiv.), Mel 7 uL (0.11 mmol, 2.2 equiv.), potassium carbonate 22 mg (0.15 mmol, 3.0 equiv.) and DMF 1 mL was added to the vial, and the reaction mixture was stirred at room temperature. The product was purified by prep-HPLC and confirmed by LCMS. Calculated M+1 C₁₉H₃₃N₄O₂S 381.23, found: 381.10.

### Example 193: N′-{2-[6-benzyloxy-1-(2-methylpropyl)-1H-indol-3-yl]ethyl}-N,N-dimethylsulfuric diamide (115)

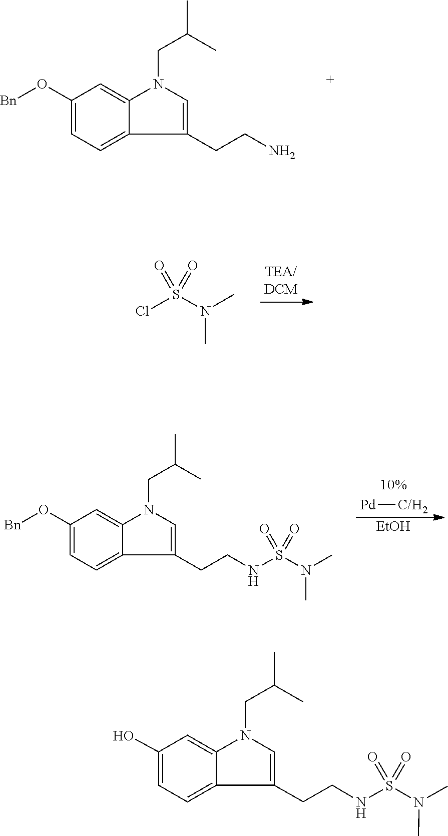

2-(6-(benzyloxy)-1-isobutyl-1H-indol-3-yl)ethan-1-amine 4.0 g (15.34 mmol, 1.0 equiv.), triethylamine 5.6 g (5.0 equiv.) and DCM 60 mL were added into the flask and the mixture was stirred at 0° C. for 30 min. Then dimethylsulfamoyl chloride 2.4 g (23 mmol, 1.5 equiv.) was added dropwise and stirred at room temperature overnight. The reaction was monitored by LCMS, 40 mL of water was added, and the organic layer was separated. The aqueous layer was extracted by DCM (50 mL×3). The combined organic layer was dried with Na₂SO₄. Solvent was removed and the residue was purified by combi-flash with ethyl acetate and hexane as eluent. 3.2 g brown oil was obtained. M+1 calculated for C₂₃H₃₂N₃O₃S 430.22, found 429.91.

### Example 194: N-{2-[6-hydroxy-1-(2-methylpropyl)-1H-indol-3-yl]ethyl}-N,N-dimethylsulfuric diamide (116)

N-{2-[6-benzyloxy-1-(2-methylpropyl)-1H-indol-3-yl]ethyl}-N,N-dimethylsulfuric diamide 900 mg (2.2 mmol, 1.0 equiv.), 10% Pd-C 100 mg and EtOH 20 mL were added to a 50 mL flask. The reaction mixture was degassed 3 times. The mixture was stirred under 1 atm H₂ atmosphere at room temperature for 20 hours. The reaction was monitored by LCMS. Solid was filtered through a celite pad and the solvent was removed under reduced pressure. The residue was purified by combi-flash, eluent (DCM:MeOH=90:10). M+1 calculated for C₁₆H₂₆N₃O₃S 340.17, found 339.83.

### Example 195: N-{2-[6-methoxyl-1-(2-methylpropyl)-1H-indol-3-yl]ethyl}-N-methyl-N,N-dimethyl sulfuric diamide (117)

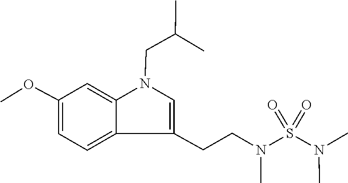

To a mixture of N′-{2-[6-hydroxy-1-(2-methylpropyl)-1H-indol-3-yl]ethyl}-N,N-dimethyl sulfuric diamide 34 mg (0.1 mmol), potassium carbonate 28 mg (0.2 mmol) and 2 mL of dry THF was added Mel 15 uL. The resulting mixture was refluxed overnight. The solid was filtered and the filtrate was purified by prep-HPLC to obtain desired product. M+1 calculated for C₁₈H₃₀N₃O₃S 368.20, found 367.83.

### Example 196: N′-{2-[6-isopropoxy-1-(2-methylpropyl)-1H-indol-3-yl]ethyl}-N,N-dimethylsulfuric diamide (118)

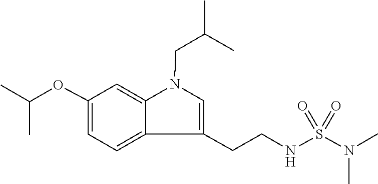 [SMILES: CC(C)Cn1cc(CCNS(=O)(=O)N(C)C)c2ccc(OC(C)C)cc21]

To a mixture of N′-{2-[6-hydroxy-1-(2-methylpropyl)-1H-indol-3-yl]ethyl}-N,N-dimethyl-sulfuric diamide 34 mg (0.1 mmol), potassium carbonate 28 mg (0.2 mmol) and 2 mL of THF was added 2-iodopropane 26 mg. The resulting mixture was refluxed overnight. The solid was filtered and the filtrate was purified by prep-HPLC to obtain desired product. M+1 calculated for C₁₉H₃₂N₃O₃S 382.22, found: 381.88.

### Example 197: N′-{2-[6-tert-butoxy-1-(2-methylpropyl)-1H-indol-3-yl]ethyl}-N,N-dimethylsulfuric diamide (119)

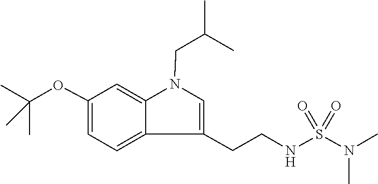 [SMILES: CC(C)Cn1cc(CCNS(=O)(=O)N(C)C)c2ccc(OC(C)(C)C)cc21]

N, N-Dimethylformamide di-t-butyl acetal (4.0 equiv) was added to a solution of N′-{2-[6-hydroxy-1-(2-methylpropyl)-1H-indol-3-yl]ethyl}-N,N-dimethylsulfuric diamide 68 mg (0.2 mmol) in dry DMF (2 ml) at 120° C., The reaction mixture was further heated for 30h. 6 mL of water was added and extracted by DCM (5 mL×3), the combined organic phase was washed with brine (5 mL×3). The solvent was removed and the residue was directly purified by prep-HPLC to obtain the desired product. M+1 calculated for C₂₀H₃₄N₃O₃S 396.23, found: 395.79.

### Examples 198 and 199

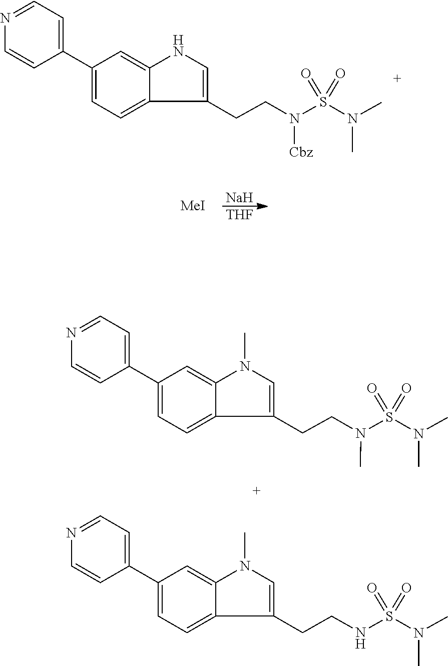

NaH (0.4 mmol) was added to a solution of N′-[2-(6-(pyridin-4-yl)-1H-indol-3-yl)ethyl]-N′-benzyl carbamate-N,N-dimethylsulfuric diamide (0.2 mmol) in dry tetrahydrofuran (THF; 1 mL) at 0° C. and the reaction mixture was stirred for 10 min. Mel (0.30 mmol) was added to the reaction mixture. The reaction mixture was warmed to room temperature and stirred for 12 hours. After the reaction was complete the reaction mixture was quenched with a few drops of water, and then purified by prep-HPLC directly.

### Example 198: N′-{2-[6-(pyridin-4-yl)-1-methyl-1H-indol-3-yl]ethyl}-N′-methyl-N,N-dimethylsulfuric diamide (127)

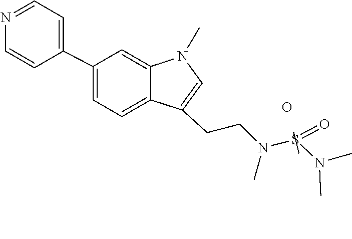

M+1 calculated for C₁₉H₂₅N₄O₂S: 373.17, found: 373.03.

### Example 199: N′-{2-[6-(pyridin-4-yl)-1-methyl-1H-indol-3-yl]ethyl}-N,N-dimethylsulfuric diamide (128)

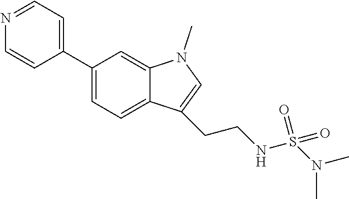 [SMILES: CN(C)S(=O)(=O)NCCc1cn(C)c2cc(-c3ccncc3)ccc12]

M+1 calculated for C₁₈H₂₃N₄O₂S 359.15, found: 358.98.

### Examples 200 and 201

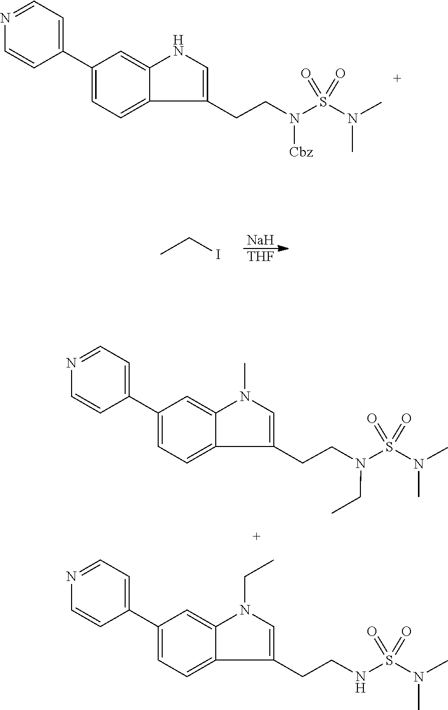

NaH (0.4 mmol) was added to a solution of N′-[2-(6-(pyridin-4-yl)-1H-indol-3-yl)ethyl]-N′-benzyl carbamate-N,N-dimethylsulfuric diamide (0.2 mmol) in dry tetrahydrofuran (THF; 1 mL) at 0° C. and the reaction mixture was stirred for 10 min. EtI (0.3 mmol) was added dropwise to the reaction mixture. The reaction mixture was warmed to room temperature and stirred for 12 hours. After the reaction was complete, the reaction mixture was quenched with a few drops of water and the reaction mixture was purified by prep-HPLC directly.

### Example 200: N′-{2-[6-(pyridin-4-yl)-1-ethyl-1H-indol-3-yl]ethyl}-N,N-dimethylsulfuric diamide (132)

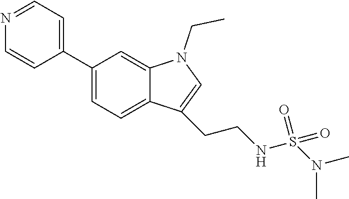 [SMILES: CCn1cc(CCNS(=O)(=O)N(C)C)c2ccc(-c3ccncc3)cc21]

M+1 calculated for C₁₉H₂₅N₄O₂S 373.17, found: 372.95.

### Example 201: N′-{2-[6-(pyridin-4-yl)-1-ethyl-1H-indol-3-yl]ethyl}-N′-ethyl-N,N-dimethylsulfuric diamide (133)

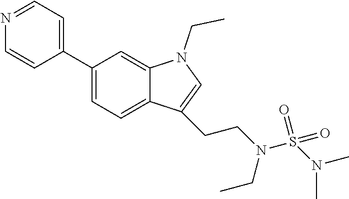 [SMILES: CCN(CCc1cn(CC)c2cc(-c3ccncc3)ccc12)S(=O)(=O)N(C)C]

M+1 calculated for C₂₁H₂₉N₄O₂S 401.20, found: 401.02.

### Example 202: N′-{2-[6-(pyridin-4-yl)-1-isopropyl-1H-indol-3-yl]ethyl}-N,N-dimethylsulfuric diamide (129)

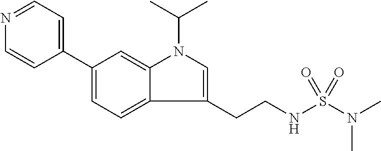 [SMILES: CC(C)n1cc(CCNS(=O)(=O)N(C)C)c2ccc(-c3ccncc3)cc21]

NaH (0.4 mmol) was added to a solution of N′-[2-(6-(pyridin-4-yl)-1H-indol-3-yl)ethyl]-N′-benzyl carbamate-N,N-dimethylsulfuric diamide (0.2 mmol) in dry tetrahydrofuran (1 mL) at 0° C. and the reaction mixture was stirred for 10 min. 2-iodopropane (0.30 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC. M+1 calculated for C₂₀H₂₇N₄O₂S 387.19, found: 387.04.

### Example 203: N′-{2-[6-(pyridin-4-yl)-1-cyclopropyl-1H-indol-3-yl]ethyl}-N,N-dimethylsulfuric diamide (134)

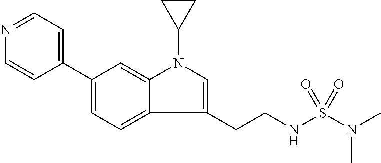 [SMILES: CN(C)S(=O)(=O)NCCc1cn(C2CC2)c2cc(-c3ccncc3)ccc12]

NaH (0.4 mmol) was added to a solution of N′-[2-(6-(pyridin-4-yl)-1H-indol-3-yl)ethyl]-N′-benzylcarbamate-N,N-dimethylsulfuric diamide (0.2 mmol) in dry tetrahydrofuran (1 mL) at 0° C. and the reaction mixture was stirred for 10 min. iodocyclopropane (0.3 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC. M+1 calculated for C₂₀H₂₅N₄O₂S 385.17, found: 385.12.

### Example 204: N′-{2-[6-(pyridin-4-yl)-1-phenethyl-1H-indol-3-yl]ethyl}-N,N-dimethylsulfuric diamide (154)

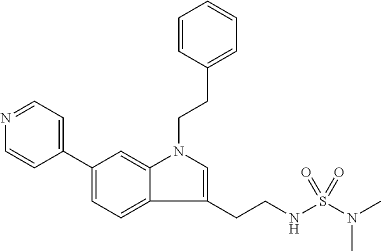 [SMILES: CN(C)S(=O)(=O)NCCc1cn(CCc2ccccc2)c2cc(-c3ccncc3)ccc12]

NaH (0.4 mmol) was added to a solution of N′-[2-(6-(pyridin-4-yl)-1H-indol-3-yl)ethyl]-N′-benzylcarbamate-N,N-dimethylsulfuric diamide (0.2 mmol) in dry tetrahydrofuran (1 mL) at 0° C. and the reaction mixture was stirred for 10 min. 2-bromoethyl)benzene (0.3 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC. M+1 calculated for C₂₅H₂₉N₄O₂S 449.20, found 449.03.

### Example 205: N′-{2-[6-(pyridin-4-yl)-1-isopropylsulfonyl-1H-indol-3-yl]ethyl}-N,N-dimethylsulfuric diamide (130)

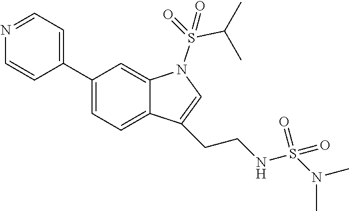 [SMILES: CC(C)S(=O)(=O)n1cc(CCNS(=O)(=O)N(C)C)c2ccc(-c3ccncc3)cc21]

NaH (0.4 mmol) was added to a solution of N′-[2-(6-(pyridin-4-yl)-1H-indol-3-yl)ethyl]-N′-benzylcarbamate-N,N-dimethylsulfuric diamide (0.2 mmol) in dry tetrahydrofuran (1 mL) at 0° C. and the reaction mixture was stirred for 10 min. Propane-2-sulfonyl chloride (0.3 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC. M+1 calculated for C₂₀H₂₇N₄O₄S₂ 451.15, found: 451.03.

### Example 206: N′-{2-[6-(pyridin-4-yl)-1-(3,5-dichlorobenzoyl)-1H-indol-3-yl]ethyl}-N,N-dimethylsulfuric diamide (151)

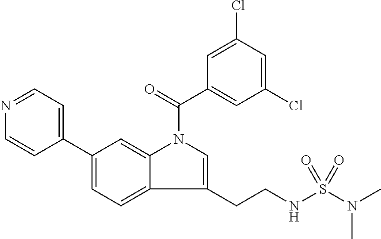 [SMILES: CN(C)S(=O)(=O)NCCc1cn(C(=O)c2cc(Cl)cc(Cl)c2)c2cc(-c3ccncc3)ccc12]

NaH (0.4 mmol) was added to a solution of N′-[2-(6-(pyridin-4-yl)-1H-indol-3-yl)ethyl]-N′-benzylcarbamate-N,N-dimethylsulfuric diamide (0.2 mmol) in dry tetrahydrofuran (1 mL) at 0° C. and the reaction mixture was stirred for 10 min. 3,5-dichlorobenzoyl chloride (0.3 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC. M+1 calculated for C₂₄H₂₃Cl₂N₄O₃S 517.09, found: 516.89.

General Procedures for the Indole Ring N-benzylation

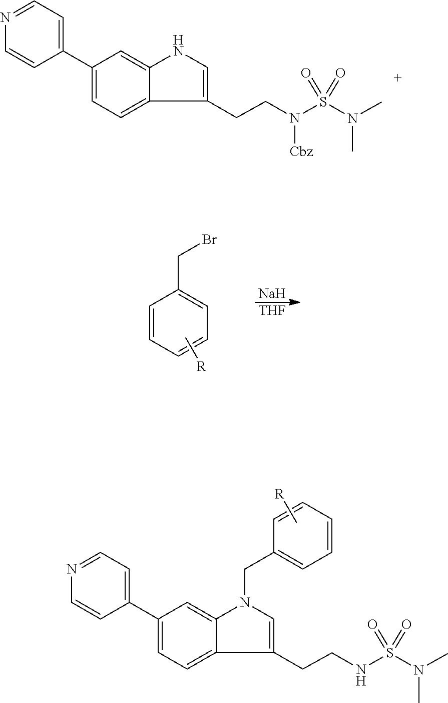

NaH (0.2 mmol) was added to a solution of N′-[2-(6-(pyridin-4-yl)-1H-indol-3-yl)ethyl]-N′-benzylcarbamate-N,N-dimethylsulfuric diamide (0.1 mmol) in dry tetrahydrofuran (1 mL) at 0° C. and the reaction mixture was stirred for 10 min. Benzyl bromide (0.15 mmol) was added dropwise to the reaction mixture. The reaction mixture was warmed to room temperature and stirred for 12 hours. After the reaction was complete the reaction mixture was quenched with a few drops of water and the crude residue was purified by prep-HPLC.

### Example 207: N′-{2-[6-(pyridin-4-yl)-1-(2-bromobenzyl)-1H-indol-3-yl]ethyl}-N,N-dimethylsulfuric diamide (140)

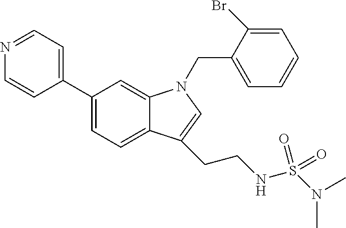 [SMILES: CN(C)S(=O)(=O)NCCc1cn(Cc2ccccc2Br)c2cc(-c3ccncc3)ccc12]

M+1 calculated for C₂₄H₂₆BrN₄O₂S 513.10, found: 512.95.

### Example 208: N-{2-[6-(pyridin-4-yl)-1-(3-chlorobenzyl)-1H-indol-3-yl]ethyl}-N,N-dimethylsulfuric diamide (141)

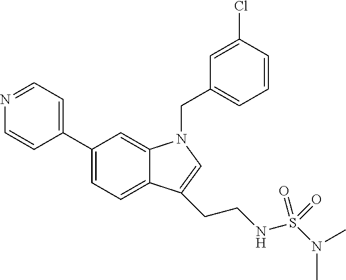 [SMILES: CN(C)S(=O)(=O)NCCc1cn(Cc2cccc(Cl)c2)c2cc(-c3ccncc3)ccc12]

M+1 calculated for C₂₄H₂₆ClN₄O₂S 469.15, found 469.02.

### Example 209: N-{2-[6-(pyridin-4-yl)-1-(3-trifluoromethylbenzyl)-1H-indol-3-yl]ethyl}-N,N-dimethyl-sulfuric diamide (142)

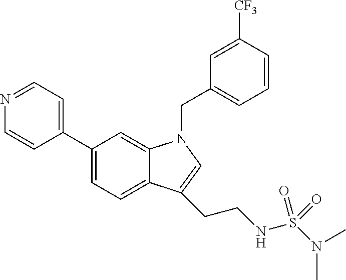 [SMILES: CN(C)S(=O)(=O)NCCc1cn(Cc2cccc(C(F)(F)F)c2)c2cc(-c3ccncc3)ccc12]

M+1 calculated for C₂₅H₂₆F₃N₄O₂S 503.17, found 503.02.

### Example 210: N-{2-[6-(pyridin-4-yl)-1-(3-bromobenzyl)-1H-indol-3-yl]ethyl}-N,N-dimethylsulfuric diamide (143)

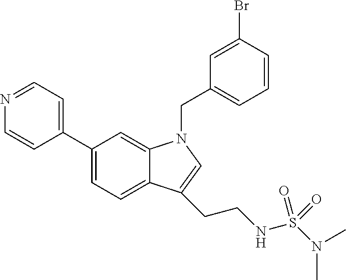 [SMILES: CN(C)S(=O)(=O)NCCc1cn(Cc2cccc(Br)c2)c2cc(-c3ccncc3)ccc12]

M+1 calculated for C₂₄H₂₆BrN₄O₂S 513.10, found: 512.96.

### Example 211: N-{2-[6-(pyridin-4-yl)-1-(3-fluorobenzyl)-1H-indol-3-yl]ethyl}-N,N-dimethylsulfuric diamide (144)

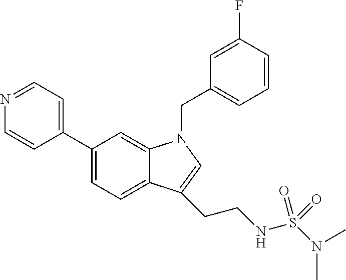 [SMILES: CN(C)S(=O)(=O)NCCc1cn(Cc2cccc(F)c2)c2cc(-c3ccncc3)ccc12]

M+1 calculated for C₂₄H₂₆FN₄O₂S 453.18, found 453.05.

### Example 212: N-{2-[6-(pyridin-4-yl)-1-(3-iodobenzyl)-1H-indol-3-yl]ethyl}-N,N-dimethylsulfuric diamide (145)

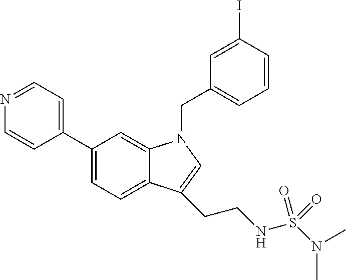 [SMILES: CN(C)S(=O)(=O)NCCc1cn(Cc2cccc(I)c2)c2cc(-c3ccncc3)ccc12]

M+1 calculated for C₂₄H₂₆IN₄O₂S 561.08, found 561.03.

### Example 213: N-{2-[6-(pyridin-4-yl)-1-(4-fluorobenzyl)-1H-indol-3-yl]ethyl}-N,N-dimethylsulfuric diamide (146)

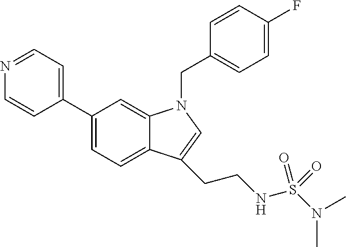 [SMILES: CN(C)S(=O)(=O)NCCc1cn(Cc2ccc(F)cc2)c2cc(-c3ccncc3)ccc12]

M+1 calculated for C₂₄H₂₆FN₄O₂S 453.18, found 453.08.

### Example 214: N-{2-[6-(pyridin-4-yl)-1-(3-methoxybenzyl)-1H-indol-3-yl]ethyl}-N,N-dimethylsulfuric diamide (147)

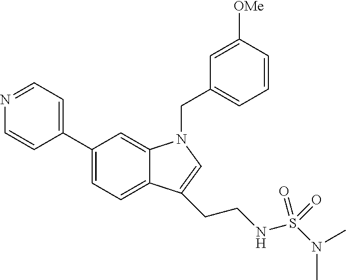 [SMILES: COc1cccc(Cn2cc(CCNS(=O)(=O)N(C)C)c3ccc(-c4ccncc4)cc32)c1]

M+1 calculated for C₂₅H₂₉N₄O₃S 465.20, found 465.08.

### Example 215: N-{2-[6-(pyridin-4-yl)-1-(3-nitrobenzyl)-1H-indol-3-yl]ethyl}-N,N-dimethylsulfuric diamide (148)

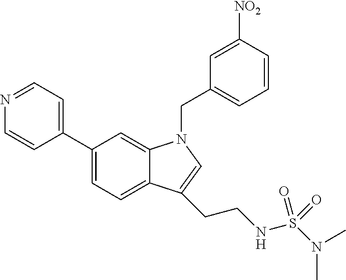 [SMILES: CN(C)S(=O)(=O)NCCc1cn(Cc2cccc([N+](=O)[O-])c2)c2cc(-c3ccncc3)ccc12]

M+1 calculated for C₂₄H₂₆N₅O₄S 480.17, found: 480.01.

### Example 216: N-{2-[6-(pyridin-4-yl)-1-(2,4-difluorobenzyl)-1H-indol-3-yl]ethyl}-N,N-dimethylsulfuric diamide (149)

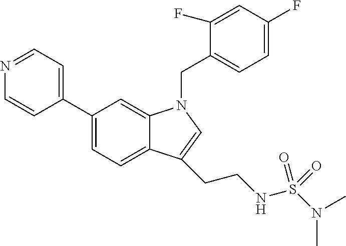 [SMILES: CN(C)S(=O)(=O)NCCc1cn(Cc2ccc(F)cc2F)c2cc(-c3ccncc3)ccc12]

M+1 calculated for C₂₄H₂₅F₂N₄O₂S 471.17, found: 471.04.

### Example 217: N-{2-[6-(pyridin-4-yl)-1-(2-fluorobenzyl)-1H-indol-3-yl]ethyl}-N,N-dimethylsulfuric diamide (150)

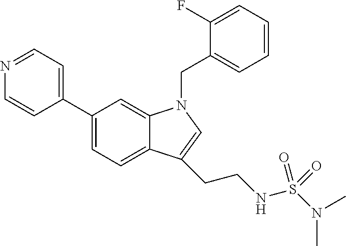 [SMILES: CN(C)S(=O)(=O)NCCc1cn(Cc2ccccc2F)c2cc(-c3ccncc3)ccc12]

M+1 calculated for C₂₄H₂₆FN₄O₂S 453.18, found 452.98.

### Example 218: N-{2-[5-chloro-3-(2,2,2-trifluoroethyl)-1H-indol-1-yl]ethyl}-N,N-dimethylsulfuric diamide (152)

### 1-(5-chloro-1H-indol-3-yl)-2,2,2-trifluoroethan-1-one

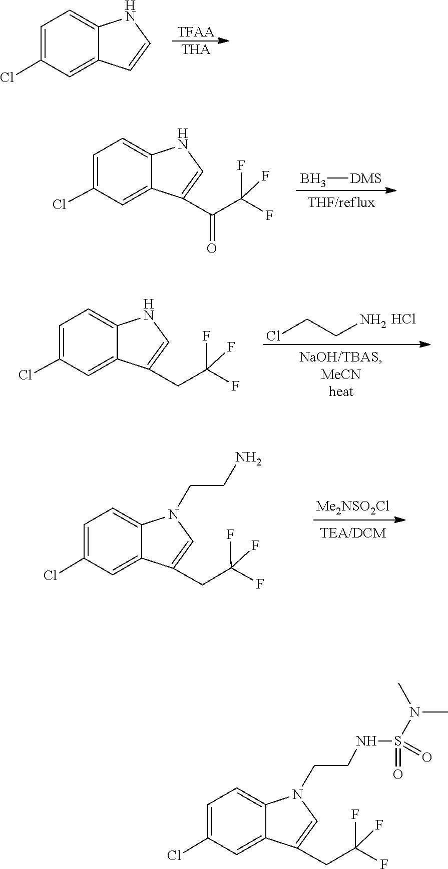

Trifluoroacetic anhydride (3.2 g, 1.5 eq) was added to a solution of 5-chloro-1H-indole (1.5 g, 1 eq) in dry THF (50 mL) at 0° C. and the reaction mixture was slowly warmed to room temperature. After completion of the reaction, the solvent was removed under vacuum and the crude product was treated with DCM and MeOH to obtain a solid. The solid was separated by filtration and washed with MeOH and dried under high vacuum. 2.0 g solid was obtained, yield 80%. M+1 calculated for 247.86, found 247.54. The solid was used in next step directly.

### 5-chloro-3-(2,2,2-trifluoroethyl)-1H-indole

Borane dimethylsulfide complex (3.0 g, 24 mmol) was added dropwise to a stirred solution of 1-(5-chloro-1H-indol-3-yl)-2,2,2-trifluoroethan-1-one (2.0 g, 8 mmol) in dry THF (35 mL) at 60° C. The reaction mixture to stirred at reflux for 7 hours, and then cooled to 0° C. The reaction was carefully quenched with MeOH (10 mL) and then concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the product. 1.6 g, yield 86%. ¹HNMR (CDCl₃, 400 Hz) 3.49 (q, J=10 Hz, 2H), 7.19 (dd, J₁=1.9 Hz, J₂=8.4 Hz, 1H), 7.21 (d, J=1.9 Hz, 1H), 7.31 (d, J=8.4 Hz, 1H), 7.58 (d, J=1.9 Hz, 1H), 8.22 (br, 1H).

### 2-(5-chloro-3-(2,2,2-trifluoroethyl)-1H-indol-1-yl)ethan-1-amine

Powdered NaOH (0.44 g, 11 mmol) and tetrabutylammonium hydrogensulfate (0.11 g, 0.4 mmol) were added to a solution of 5-chloro-3-(2,2,2-trifluoroethyl)-1H-indole (1.2 g, 5 mmol) in dry CH₃CN (30 mL). After stirring at 25° C. for 30 minutes, 2-chloroethylamine hydrochloride (1.3 g, 11 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the product 350 mg, yield 25%. M+1 calculated for C₁₂H₁₃ClF₃N₂ 277.06, found 276.82.

### N′-{2-[5-chloro-3-(2,2,2-trifluoroethyl)-1H-indol-1-yl]ethyl}-N,N-dimethylsulfuric diamide (152)

To a solution of 2-(5-chloro-3-(2,2,2-trifluoroethyl)-1H-indol-1-yl)ethan-1-amine 30 mg (0.1 mmol) and triethylamine (30 uL) in DCM (1 mL) was added dimethylsulfamoyl chloride (21 mg, 0.15 mmol) at 0° C. The reaction was warmed to room temperature and stirred overnight. The reaction was concentrated in vacuo, and the crude residue was purified by prep-HPLC. M+1 calculated for C₁₄H₁₈ClF₃N₃O₂S 384.08, found: 383.77.

### Example 219: N-{2-[5-chloro-3-(2,2,3,3,3-pentafluoropropyl)-1H-indol-1-yl]ethyl}-N,N-dimethylsulfuric diamide (153)

### 1-(5-chloro-1H-indol-3-yl)-2,2,3,3,3-pentafluoropropan-1-one

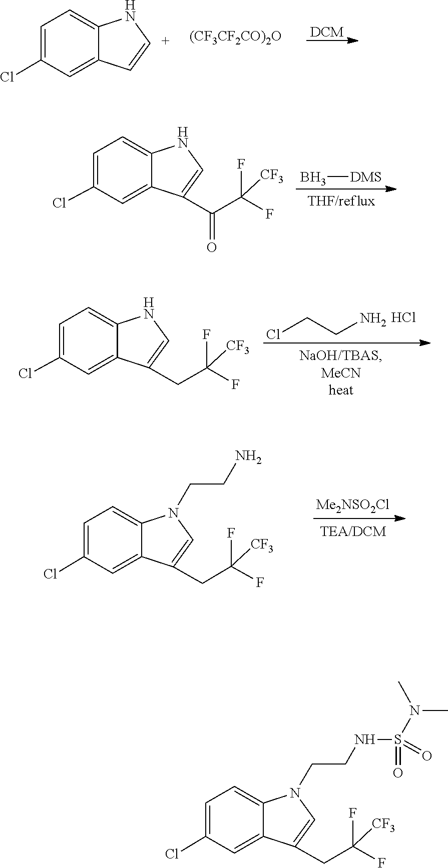

2,2,3,3,3-pentafluoropropanoic anhydride (4.5 g, 1.5 eq) was added to a solution of 5-chloro-1H-indole (1.5 g, 1 eq) in dry THF (50 mL) at 0° C. and the reaction mixture was slowly warmed to room temperature. After completion of the reaction, the solvent was removed under vacuum and the resulting oil was treated with DCM and MeOH to obtain a solid. The solid was separated by filtration and washed with MeOH and dried under high vacuum. 2.4 g solid was obtained, yield 80%. M+1 calculated for C₁₁H₆ClF₅NO 297.68, found 297.45. ¹HNMR (CDCl₃, 400 Hz) 7.35 (dd, J₁=1.0 Hz, J₂=8.6 Hz, 1H), 7.41 (dd, J₁=2.0 Hz, J₂=8.6 Hz, 1H), 8.45 (d, J=2.0 Hz, 1H), 8.13 (m, 1H), 8.96 (br, 1H).

### 5-chloro-3-(2,2,3,3,3-pentafluoropropyl)-1H-indole

Borane dimethylsulfide complex (3.0 g, 24 mmol) was added in a dropwise manner to a stirred solution of 1-(5-chloro-1H-indol-3-yl)-2,2,3,3,3-pentafluoropropan-1-one (2.4 g, 8 mmol) in dry THF (50 mL) at 60° C. The reaction mixture was stirred at reflux for 7 hours, and then cooled to 0° C. MeOH (10 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the product 1.5 g, yield 66%. M+1 calculated for C₁₁H₈ClF₅N 283.82, found 283.65.

### 2-(5-chloro-3-(2,2,3,3,3-pentafluoropropyl)-1H-indol-1-yl)ethan-1-amine

Powdered NaOH (0.44 g, 11 mmol) and tetrabutylammonium hydrogensulfate (0.11 g, 0.4 mmol) were added to a solution of 5-chloro-3-(2,2,3,3,3-pentafluoropropyl)-1H-indole (1.4 g, 5 mmol) in dry CH₃CN (30 mL). After stirring at 25° C. for 30 minutes, 2-chloroethylamine hydrochloride (1.3 g, 11 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the product 420 mg, yield 26%. M+1 calculated for C₁₃H₁₂ClF₅N₂ 326.86, found 326.54.

### N′-{2-[5-chloro-3-(2,2,3,3,3-pentafluoropropyl)-1H-indol-1-yl]ethyl}-N,N-dimethylsulfuric diamide (153)

To a solution of 2-(5-chloro-3-(2,2,3,3,3-pentafluoropropyl)-1H-indol-1-yl)ethan-1-amine 33 mg (0.1 mmol) and triethylamine (30 uL) in DCM (1 mL) was added dimethylsulfamoyl chloride (21 mg, 0.15 mmol) at 0° C. The reaction was warmed to room temperature and stirred overnight. The reaction was concentrated in vacuo, and the crude residue was purified by prep-HPLC. M+1 calculated for C₁₅H₁₈ClF₅N₃O₂S 434.07, found: 433.71.

### Example 220: N-{2-[5-bromo-3-(3,3,3-trifluoropropyl)-1H-indol-1-yl]ethyl}-N,N-dimethylsulfuric diamide (172)

### 1-(5-bromo-1H-indol-3-yl)-3,3,3-trifluoropropan-1-one

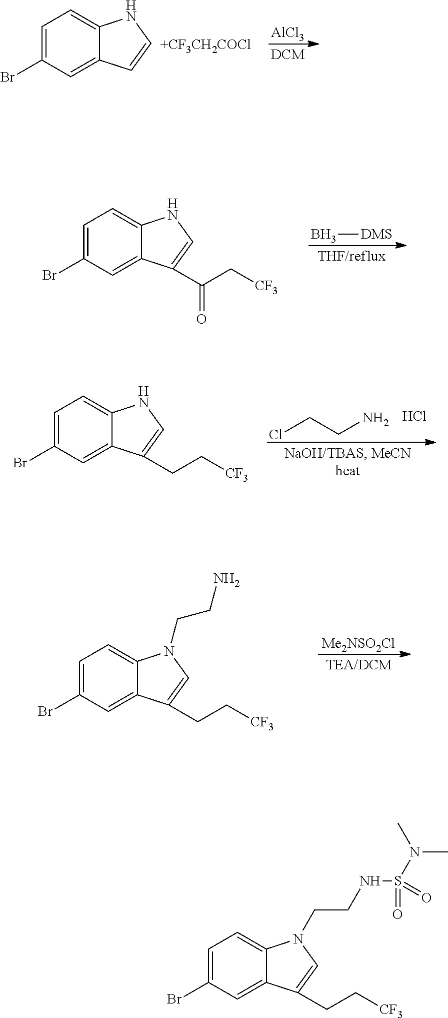

3,3,3-trifluoropropanoyl chloride (1.6 g, 1.1 equiv.) was added to a solution of 5-bromo-1H-indole (1.95 g, 1 equiv.) and AlCl₃ (1.40 g, 1.1 equiv.) in dry DCM (50 mL) at 0° C. under N₂ protection and the reaction mixture was slowly warmed to room temperature. The reaction mixture was poured into 30 mL of water, the solid was filtered through a celite pad and washed with DCM (25 mL). The aqueous layer was separated and the organic layer was dried with Na₂SO₄. The solvent was removed under reduced pressure and the residue was purified by combi-flash to obtain 1.2 g oil, yield 40%. M+1 calculated for C₁₁H₇BrF₃NO 305.76, found 305.61.

### 5-bromo-3-(3,3,3-trifluoropropyl)-1H-indole

Borane dimethylsulfide complex (1.5 g, 12 mmol) was added in a dropwise manner to a stirred solution of 1-(5-bromo-1H-indol-3-yl)-3,3,3-trifluoropropan-1-one (1.2 g, 4 mmol) in dry THF (30 mL) at 60° C. The reaction mixture was stirred at reflux for 7 hours, and then cooled to 0° C. MeOH (50 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the product, 0.8 g, yield 67%. M+1 calculated for C₁₁H₉BrF₃N 291.87, found 291.65. ¹HNMR (CDCl₃, 400 Hz) 2.40-2.52 (m, 2H), 2.94-3.01 (m, 2H), 7.03 (t, J=1.2 Hz, 1H), 7.24 (dd, J₁=1.8 Hz, J₂=8.6 Hz, 1H), 7.30 (dd, J₁=1.8 Hz, J₂=8.6 Hz, 1H), 7.69 (m, 1H), 8.05 (br, 1H).

### 2-(5-bromo-3-(3,3,3-trifluoropropyl)-1H-indol-1-yl)ethan-1-amine

Powdered NaOH (0.17 g, 4.4 mmol) and tetrabutylammonium hydrogensulfate (0.06 g, 0.2 mmol) were added to a solution of 5-bromo-3-(3,3,3-trifluoropropyl)-1H-indole (0.6 g, 2 mmol) in dry CH₃CN (10 mL). After stirring at 25° C. for 30 minutes, 2-chloroethylamine hydrochloride (0.6 g, 4 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the product 210 mg, yield 31%. M+1 calculated for C₁₃H₁₅BrF₃N₂ 334.83, found 334.48.

### N′-{2-[5-bromo-3-(3,3,3-trifluoropropyl)-1H-indol-1-yl]ethyl}-N,N-dimethylsulfuric diamide (172)

To a solution of 2-(5-bromo-3-(3,3,3-trifluoropropyl)-1H-indol-1-yl)ethan-1-amine 33 mg (0.1 mmol) and triethylamine (30 uL) in DCM (1 mL) was added dimethylsulfamoyl chloride (21 mg, 0.15 mmol) at 0° C. The reaction was warmed to room temperature and stirred overnight. The reaction was concentrated in vacuo, and the crude residue was purified by prep-HPLC. M+1 calculated for C₁₅H₂₀BrF₃N₃O₂S 442.01, found: 441.80.

### Example 221: N-(2-(5-bromo-3-neopentyl-1H-indol-1-yl)ethyl)propane-2-sulfonamide (171)

### 1-(5-bromo-1H-indol-3-yl)-2,2-dimethylpropan-1-one

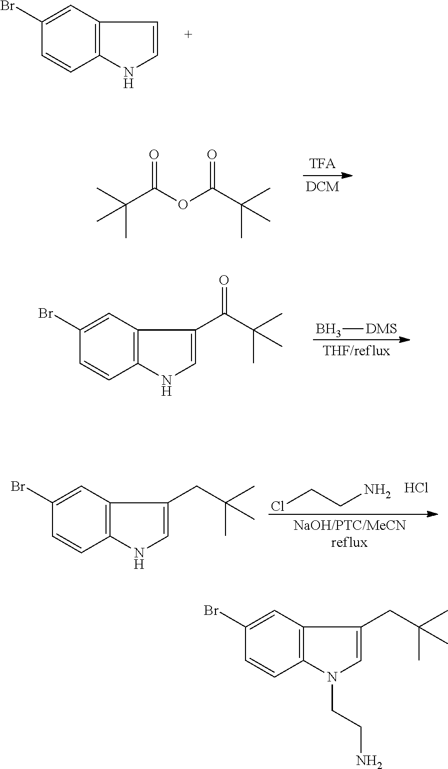

Pivalic anhydride (14.0 g, 1.5 eq) was added to a solution of 5-bromo-1H-indole (9.5 g, 1 eq) in dry DCM (150 mL) and TFA (20 mL) at 0° C. and the reaction mixture was slowly warmed to room temperature. After completion of the reaction, the solvent was removed under vacuum and the resulting oil was treated with DCM and MeOH to obtain a solid. The solid was separated by filtration and washed with MeOH and dried under high vacuum. 10.0 g solid was obtained, yield 71%. M+1 calculated for C₁₃H₁₅BrNO 280.83, found 280.51. ¹HNMR (CDCl₃, 400 Hz) 1.43 (s, 9H), 7.27 (d, J=8.0 Hz, 1H), 7.35 (dd, J₁=2.0 Hz, J₂=8.4 Hz, 1H), 7.94 (d, J=3.0 Hz, 1H), 7.69 (m, 1H), 8.59 (br, 1H), 8.70 (d, J=2.0 Hz, 1H)

### 5-bromo-3-neopentyl-1H-indole

Borane dimethylsulfide complex (13.5 g, 108 mmol) was added in a dropwise manner to a stirred solution of 1-(5-bromo-1H-indol-3-yl)-2,2-dimethylpropan-1-one (10.0 g, 36 mmol) in dry THF (120 mL) at 60° C. The reaction mixture was stirred at reflux for 24 hours, and then cooled to 0° C. MeOH (20 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the product 8.1 g, yield 87%. M+1 calculated for C₁₃H₁₇BrN 265.85, found 265.53. ¹HNMR (CDCl₃, 400 Hz) 0.97 (s, 9H), 2.58 (s, 2H), 6.96 (d, J=2.4 Hz, 1H), 7.22 (d, J=1.0 Hz, 1H), 7.23 (d, J=2.0 Hz, 1H), 7.71 (m, 1H), 8.03 (br, 1H).

### 2-(5-bromo-3-neopentyl-1H-indol-1-yl)ethan-1-amine

Powdered NaOH (0.17 g, 4.4 mmol) and tetrabutylammonium hydrogensulfate (0.06 g, 0.2 mmol) were added to a solution of 5-bromo-3-neopentyl-1H-indole (0.6 g, 2 mmol) in dry CH₃CN (10 mL). After stirring at 25° C. for 30 minutes, 2-chloroethylamine hydrochloride (0.6 g, 4 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the product 210 mg, yield 31%. M+1 calculated for C₁₃H₁₅BrF₃N₂ 334.83, found 334.48.

### N-(2-(5-bromo-3-neopentyl-1H-indol-1-yl)ethyl)propane-2-sulfonamide (171)

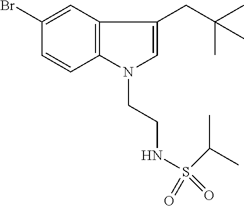 [SMILES: CC(C)S(=O)(=O)NCCn1cc(CC(C)(C)C)c2cc(Br)ccc21]

To a solution of 2-(5-bromo-3-neopentyl-1H-indol-1-yl)ethan-1-amine (33 mg, 0.1 mmol) and triethylamine (20 mg, 0.2 mmol) in dry DCM was added propane-2-sulfonyl chloride (21 mg, 0.15 mmol) at 0° C. The reaction mixture was stirred at room temperature for 12h and then purified by prep-HPLC to obtain the title compound. M+1 calculated for C₁₈H₂₈BrN₂O₂S 415.11, found 415.84.

### Example 222: N-(2-(5-bromo-3-neopentyl-1H-indol-1-yl)ethyl)cyclopropanesulfonamide (298)

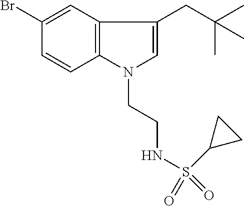 [SMILES: CC(C)(C)Cc1cn(CCNS(=O)(=O)C2CC2)c2ccc(Br)cc12]

To a solution of 2-(5-bromo-3-neopentyl-1H-indol-1-yl)ethan-1-amine (3.3 g, 10 mmol) and triethylamine (2.0 g, 20 mmol) in dry DCM (50 mL) was added cyclopropanesulfonyl chloride (2.1 g, 15 mmol) at 0° C. The reaction mixture was stirred at room temperature for 12 hours. After the reaction was complete, 50 mL water was poured into the flask. The layers were separated, and the aqueous layer was extracted with DCM (25 mL×3). The combined organics were dried (MgSO₄), concentrated and purified by combi-flash to obtain 3.2 g brown oil. M+1 calculated for C₁₈H₂₆BrN₂O₂S 413.09, found 412.82.

### Examples 223 and 224

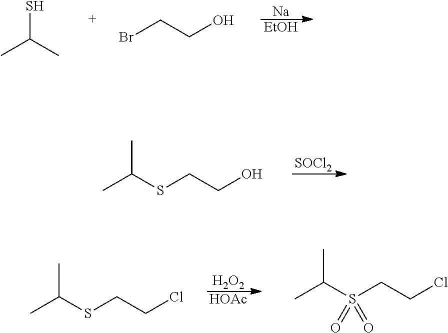

### 2-(isopropylthio)ethan-1-ol

1-methylethyl thiol (6.3 g, 82 mmol) was added dropwise to a solution of sodium (1.9 g, 82 mmol) in absolute ethanol (100.0 mL) in a 250 mL oven-dried round-bottom flask. The mixture was stirred for 1h. 2-bromoethanol (6.66 g, 82 mmol) was slowly added to the reaction mixture. The reaction mixture was stirred at room temperature overnight. The solvent was removed under reduced pressure slowly to obtain a crude oil. The solid NaBr was filtered and the crude product was used without further purification. ¹HNMR (CDCl₃, 400 Hz) 1.26 (d, J=7.8 Hz, 6H), 2.73-2.77 (m, 2H), 2.90-2.98 (m, 1H), 3.64-3.74 (m, 2H).

### (2-chloroethyl)(isopropyl)sulfane

A solution of 2-(isopropylthio)ethan-1-ol (2.4 g, 20 mmol) in 20 ml of thionyl chloride was heated to reflux for 2 hours. The reaction mixture was cooled to room temperature, and the excess thionyl chloride was removed under reduced pressure. DCM (20 mL) was added to the crude oil and the solution was concentrated in vacuo (2×). The crude residue was purified by combi-flash to obtain 1.8 g of an oil. ¹HNMR (CDCl₃, 400 Hz) 1.26 (d, J=8.0 Hz, 6H), 2.88 (q, J=8.1 Hz, 2H), 2.99 (m, 1H), 3.63 (q, J=8.1 Hz, 2H).

### 2-((2-chloroethyl)sulfonyl)propane

To a solution of (2-chloroethyl)(isopropyl)sulfane 1.8 g (1.3 mmol) in acetic acid (10 mL) was added dropwise at 0° C. 30% aqueous hydrogen peroxide (2.4 eq.). The reaction mixture was stirred for 12 h during which time the temperature rose to room temperature. Removal of the solvent and excess oxidizing agent under reduced pressure afforded the crude sulfone. ¹HNMR (CDCl₃, 400 Hz) 1.43 (d, J=8.0 Hz, 6H), 3.19-3.29 (m, 1H), 3.40 (t, J=7.1 Hz, 2H), 3.93 (t, J=7.1 Hz, 2H).

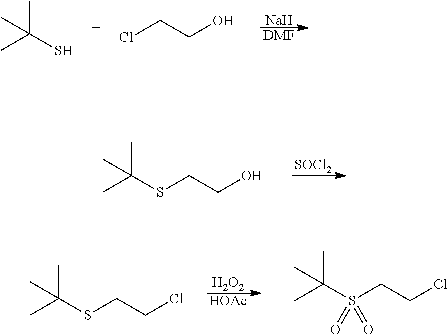

### 2-(tert-butylthio)ethan-1-ol

Sodium hydride (0.2 g, 50 mmol) was added to a solution of 2-methylpropane-2-thiol (4.5 g, 50 mmol) in anhydrous DMF (50.0 mL) in a 100 mL oven-dried round-bottom flask. The mixture was stirred for 1h. Then 2-chloroethanol (4.0 g, 50 mmol) was slowly added to the reaction mixture. The reaction mixture was stirred at room temperature overnight. The solvent was removed under reduced pressure slowly to obtain the crude residue. The solid NaCl was filtered and the crude product was purified by combi-flash to obtain 5.2 g oil which was used without further purification. ¹HNMR (CDCl₃, 400 Hz) 1.34 (s, 9H), 2.78 (t, J=6.4 Hz, 2H), 3.74 (m, 2H).

### tert-butyl(2-chloroethyl)sulfane

A solution of 2-(tert-butylthio)ethan-1-ol (2.7 g, 20 mmol) in 20 ml of thionyl chloride was heated under reflux for 2 hours. The reaction mixture was cooled to room temperature, and the excess thionyl chloride was removed under reduced pressure. DCM (20 mL) was added to the crude oil and the solution was concentrated in vacuo (2×). The residue was purified by combi-flash to obtain 1.9 g oil. ¹HNMR (CDCl₃, 400 Hz) 1.34 (s, 9H), 2.86-2.90 (m, 2H), 3.59-3.63 (m, 2H).

### 2-((2-chloroethyl)sulfonyl)-2-methylpropane

To a solution of tert-butyl(2-chloroethyl)sulfane 1.9 g (1.2 mmol) in acetic acid (10 mL) was added dropwise at 0° C. 30% aqueous hydrogen peroxide (2.4 eq.). The reaction mixture was stirred for 12 h during which time the temperature rose to room temperature. Removal of the solvent and excess oxidizing agent under reduced pressure afforded the crude sulfone. ¹HNMR (CDCl₃, 400 Hz) 1.42 (s, 9H), 3.57 (t, J=7.6 Hz, 2H), 3.95 (t, J=7.6 Hz, 2H).

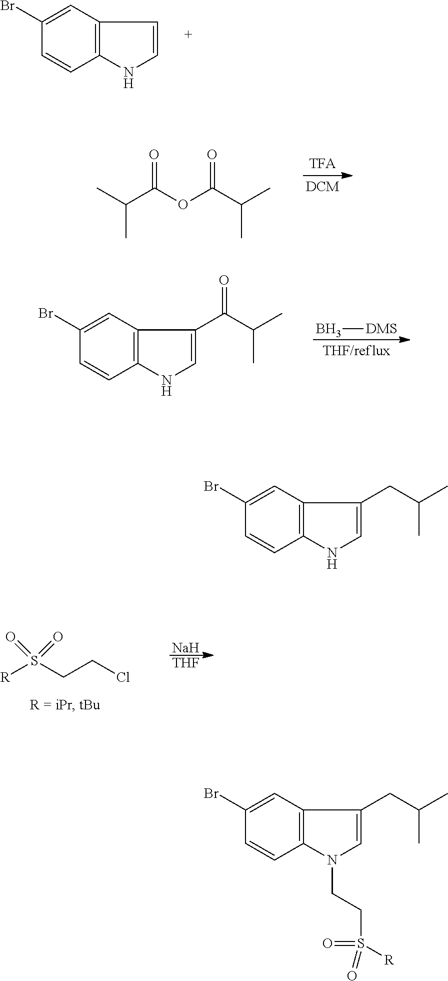

### 1-(5-bromo-1H-indol-3-yl)-2-methylpropan-1-one

Isobutyric anhydride (4.8 g, 30 mmol) was added to a solution of 5-bromo-1H-indole (3.8 g, 20 mmol) in dry DCM (50 mL) and TFA (10 mL) at 0° C. and the reaction mixture was slowly warmed to room temperature. After completion of the reaction, the solvent was removed under vacuum and the resulting oil was treated with DCM and MeOH to obtain a solid. The solid was separated by filtration and washed with MeOH and dried under high vacuum to afford 4.1 g solid, yield 75%. M+1 calculated for C₁₂H₁₃BrNO 265.81, found 265.24.

### 5-bromo-3-isobutyl-1H-indole

Borane dimethylsulfide complex (5.5 g, 45 mmol) was added in a dropwise manner to a stirred solution of 1-(5-bromo-1H-indol-3-yl)-2-methylpropan-1-one (4.1 g, 15.4 mmol) in dry THF (60 mL) at 60° C. The reaction mixture was stirred at reflux for 24 hours, and then cooled to 0° C. MeOH (20 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the product 2.9 g, yield 76%. M+1 calculated for C₁₂H₁₅BrN 251.83, found 251.48. ¹HNMR (CDCl3, 400 Hz) 0.93 (s, 3H), 0.95 (s, 3H), 1.96 (m, 1H), 2.57 (dd, J₁=0.7 Hz, J₂=7.1 Hz, 2H), 6.97 (d, J=2.5 Hz, 1H), 7.23 (d, J=0.7 Hz, 1H), 7.24 (d, J=2.0 Hz, 1H), 7.71 (t, J=0.7 Hz, 1H), 7.96 (br, 1H).

### Example 223: 5-bromo-3-isobutyl-1-(2-(isopropylsulfonyl)ethyl)-1H-indole (173)

To a solution of 5-bromo-3-isobutyl-1H-indole (50 mg, 0.2 mmol) in dry THF (1 mL) was added NaH (10 mg) at 0° C. After stirring for 30 min, (2-chloroethyl) (isopropyl)sulfane (50 mg, 0.3 mmol) was added. The reaction mixture was stirred at room temperature overnight and purified by prep-HPLC. M+1 calculated for C₁₇H₂₅BrNO₂S 386.08, found 385.96.

### Example 224: 5-bromo-1-(2-(tert-butylsulfonyl)ethyl)-3-isobutyl-1H-indole (174)

To a solution of 5-bromo-3-isobutyl-1H-indole (50 mg, 0.2 mmol) in dry THF (1 mL) was added NaH (10 mg) at 0° C. After stirring for 30 min, 2-((2-chloroethyl)sulfonyl)-2-methylpropane (55 mg, 0.3 mmol) was added. The reaction mixture was stirred at room temperature overnight and purified by prep-HPLC. M+1 calculated for C₁₈H₂₇BrNO₂S 400.09, found: 399.87.

### Example 225: 5-bromo-1-(2-(isopropylsulfonyl)ethyl)-3-neopentyl-1H-indole (175)

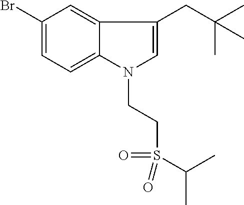 [SMILES: CC(C)S(=O)(=O)CCn1cc(CC(C)(C)C)c2cc(Br)ccc21]

To a solution of 5-bromo-3-neopentyl-1H-indole (53 mg, 0.2 mmol) in dry THF (1 mL) was added NaH (10 mg) at 0° C. After stirring for 30 min, (2-chloroethyl) (isopropyl)sulfane (50 mg, 0.3 mmol) was added. The reaction mixture was stirred at room temperature overnight and purified by prep-HPLC. M+1 calculated for C₁₈H₂₇BrNO₂S 400.09, found 399.78.

### Example 226: N-{2-[5-bromo-3-(2,2-dimethylpropyl)-1H-indol-1-yl]ethyl}-N,N-dimethylsulfuric diamide

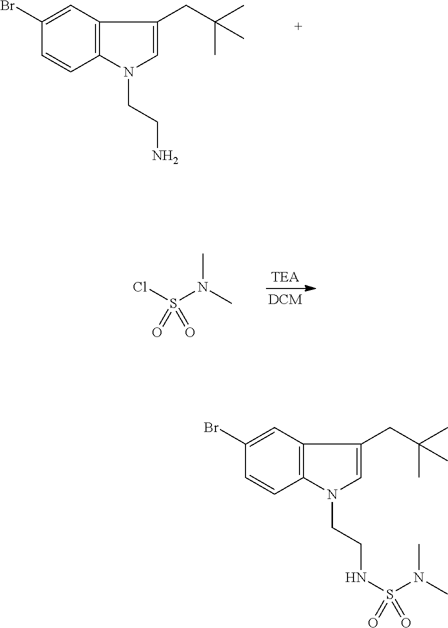

To a solution of 2-(5-bromo-3-neopentyl-1H-indol-1-yl)ethan-1-amine (200 mg, 0.65 mmol) and triethylamine (100 uL, 1.3 mmol) in 5 mL DCM was added dimethylsulfamoyl chloride (143 mg, 1 mmol) at 0° C. The reaction mixture was stirred at room temperature overnight. The solvent was removed under reduced pressure and the residue was purified by combi-flash to obtain 190 mg, yield 70%. M+1 calculated for C₁₇H₂₇BrN₃O₂S 415.89, found 415.51.

### Example 227: N-{2-[5-(pyridin-4-yl)-3-(2,2-dimethylpropyl)-1H-indol-1-yl]ethyl}-N,N-dimethylsulfuric diamide (189)

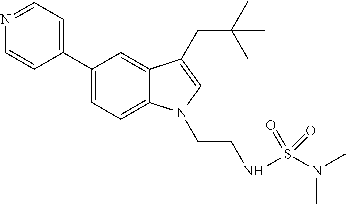 [SMILES: CN(C)S(=O)(=O)NCCn1cc(CC(C)(C)C)c2cc(-c3ccncc3)ccc21]

M+1 calculated for C₂₂H₃₁BN₄O₂S 415.22, found 415.11.

### Example 228: N-{2-[5-(2-chlorophenyl)-3-(2,2-dimethylpropyl)-1H-indol-1-yl]ethyl}-N,N-dimethyl-sulfuric diamide (190)

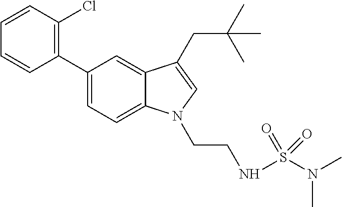 [SMILES: CN(C)S(=O)(=O)NCCn1cc(CC(C)(C)C)c2cc(-c3ccccc3Cl)ccc21]

M+1 calculated for C₂₃H₃₁ClN₃O₂S 448.18, found 447.90.

General Procedures for the Suzuki Coupling

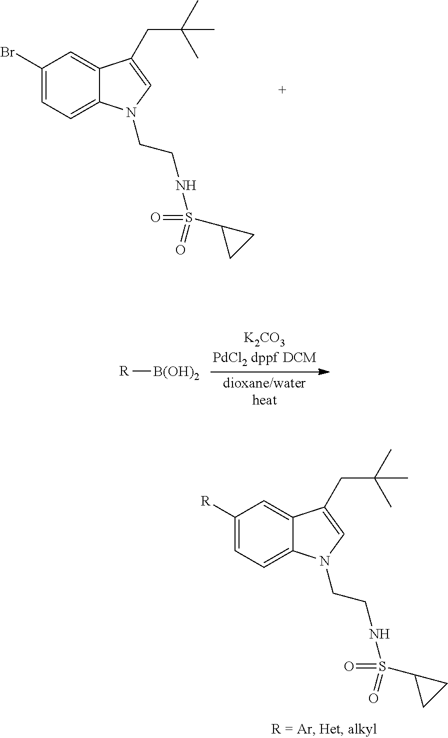

To a mixture of N-{2-[5-bromo-3-(2,2-dimethylpropyl)-1H-indol-1-yl]ethyl}cyclopropane-sulfonamide 32 mg (0.08 mmol), boronic acid (0.12 mmol) and potassium carbonate 22 mg (0.16 mmol) in 1 mL of dioxane and water (v/v=1:1) was added 3 mg of Pd catalyst. The mixture was heated at 100° C. and monitored by LCMS. The solid was filtered and the filtrate was purified by prep-HPLC to obtain the desired product.

### Example 229: N-(2-(5-(naphthalen-1-yl)-3-neopentyl-1H-indol-1-yl)ethyl)cyclopropane-sulfonamide (209)

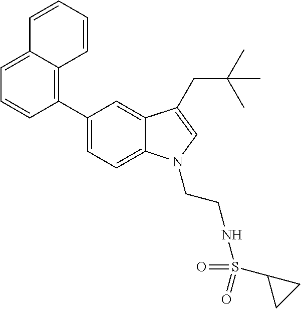 [SMILES: CC(C)(C)Cc1cn(CCNS(=O)(=O)C2CC2)c2ccc(-c3cccc4ccccc34)cc12]

M+1 calculated for C₂₈H₃₃N₂O₂S 461.23, found 460.92.

### Example 230: N-(2-(5-(naphthalen-2-yl)-3-neopentyl-1H-indol-1-yl)ethyl)cyclopropane-sulfonamide (210)

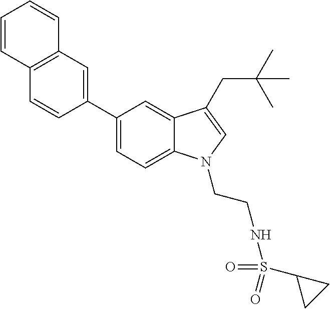 [SMILES: CC(C)(C)Cc1cn(CCNS(=O)(=O)C2CC2)c2ccc(-c3ccc4ccccc4c3)cc12]

M+1 calculated C₂₈H₃₃N₂O₂S 461.23, found 461.10.

### Example 231: N-(2-(5-(6-hydroxynaphthalen-2-yl)-3-neopentyl-1H-indol-1-yl)ethyl)cyclopropane sulfonamide (211)

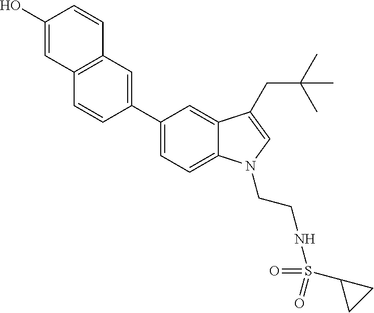 [SMILES: CC(C)(C)Cc1cn(CCNS(=O)(=O)C2CC2)c2ccc(-c3ccc4cc(O)ccc4c3)cc12]

M+1 calculated for C₂₈H₃₃N₂O₂S 477.22, found 476.91.

### Example 232: N-(2-(3-neopentyl-5-(2-(trifluoromethyl)phenyl)-1H-indol-1-yl)ethyl)cyclopropane sulfonamide (212)

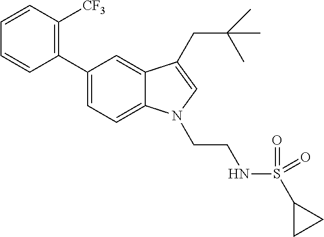 [SMILES: CC(C)(C)Cc1cn(CCNS(=O)(=O)C2CC2)c2ccc(-c3ccccc3C(F)(F)F)cc12]

M+1 calculated for C₂₅H₃₀F₃N₂O₂S 479.20, found 478.95.

### Example 233: N-(2-(5-(2-chlorophenyl)-3-neopentyl-1H-indol-1-yl)ethyl)cyclopropanesulfonamide (213)

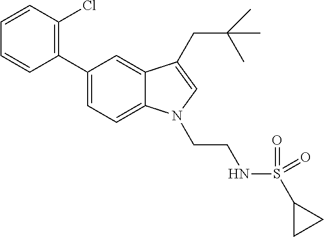 [SMILES: CC(C)(C)Cc1cn(CCNS(=O)(=O)C2CC2)c2ccc(-c3ccccc3Cl)cc12]

M+1 calculated for C₂₄H₃₀ClN₂O₂S 445.17, found 444.92.

### Example 234: N-(2-(5-(2,6-dichlorophenyl)-3-neopentyl-1H-indol-1-yl)ethyl)cyclopropane-sulfonamide (214)

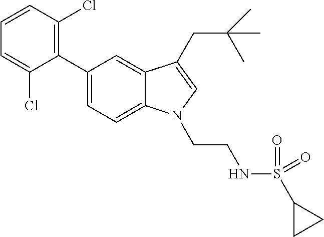 [SMILES: CC(C)(C)Cc1cn(CCNS(=O)(=O)C2CC2)c2ccc(-c3c(Cl)cccc3Cl)cc12]

M+1 calculated for C₂₄H₂₉Cl₂N₂O₂S 479.13, found 478.87.

### Example 235: N-(2-(5-(isoquinolin-1-yl)-3-neopentyl-1H-indol-1-yl)ethyl)cyclopropane-sulfonamide (215)

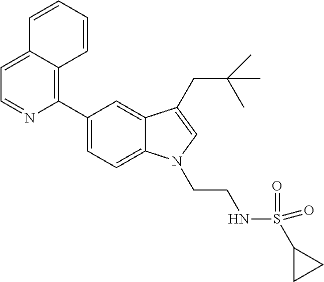 [SMILES: CC(C)(C)Cc1cn(CCNS(=O)(=O)C2CC2)c2ccc(-c3nccc4ccccc34)cc12]

M+1 calculated for C₂₇H₃₂N₃O₂S 462.22, found: 462.09.

### Example 236: N-(2-(3-neopentyl-5-(quinolin-8-yl)-1H-indol-1-yl)ethyl)cyclopropanesulfonamide (216)

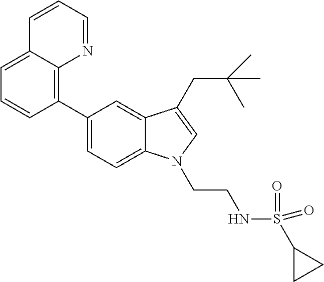 [SMILES: CC(C)(C)Cc1cn(CCNS(=O)(=O)C2CC2)c2ccc(-c3cccc4cccnc34)cc12]

M+1 calculated for C₂₇H₃₂N₃O₂S 462.22, found: 462.03.

### Example 237: N-(2-(5-(isoquinolin-8-yl)-3-neopentyl-1H-indol-1-yl)ethyl)cyclopropane-sulfonamide (217)

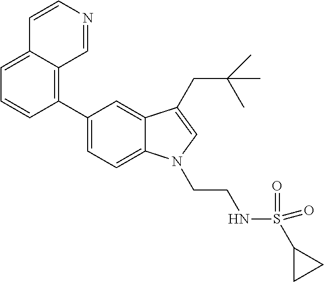 [SMILES: CC(C)(C)Cc1cn(CCNS(=O)(=O)C2CC2)c2ccc(-c3cccc4ccncc34)cc12]

M+1 calculated for C₂₇H₃₂N₃O₂S 462.22, found 462.05.

### Example 238: N-(2-(5-(isoquinolin-4-yl)-3-neopentyl-1H-indol-1-yl)ethyl)cyclopropane-sulfonamide (218)

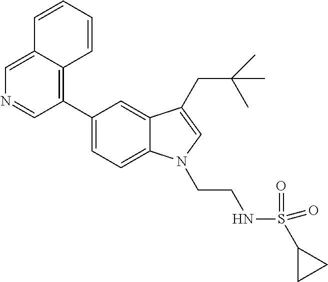 [SMILES: CC(C)(C)Cc1cn(CCNS(=O)(=O)C2CC2)c2ccc(-c3cncc4ccccc34)cc12]

M+1 calculated for C₂₇H₃₂N₃O₂S 462.22, found 462.04.

### Example 239: N-(2-(5-(4-fluoronaphthalen-1-yl)-3-neopentyl-1H-indol-1-yl)ethyl)cyclopropanesulfonamide (219)

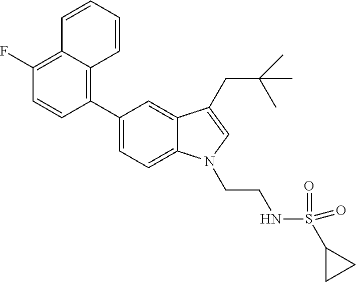 [SMILES: CC(C)(C)Cc1cn(CCNS(=O)(=O)C2CC2)c2ccc(-c3ccc(F)c4ccccc34)cc12]

M+1 calculated for C₂₇H₃₂FN₂O₂S 479.22, found 478.96.

### Example 240: N-(2-(3-neopentyl-5-phenyl-1H-indol-1-yl)ethyl)cyclopropanesulfonamide (225)

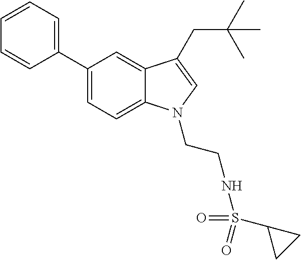 [SMILES: CC(C)(C)Cc1cn(CCNS(=O)(=O)C2CC2)c2ccc(-c3ccccc3)cc12]

M+1 calculated for C₂₄H₃₁N₂O₂S 411.21, found 410.98.

### Example 241: N-(2-(5-(3,4-difluorophenyl)-3-neopentyl-1H-indol-1-yl)ethyl)cyclopropane-sulfonamide (226)

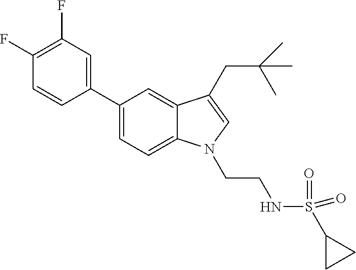 [SMILES: CC(C)(C)Cc1cn(CCNS(=O)(=O)C2CC2)c2ccc(-c3ccc(F)c(F)c3)cc12]

M+1 calculated for C₂₄H₂₉F₂N₂O₂S 447.19, found 446.88.

### Example 242: N-(2-(5-(3-fluoro-2-(trifluoromethyl)phenyl)-3-neopentyl-1H-indol-1-yl)ethyl) cyclopropanesulfonamide (227)

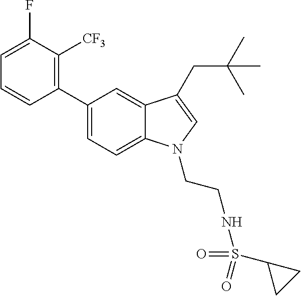 [SMILES: CC(C)(C)Cc1cn(CCNS(=O)(=O)C2CC2)c2ccc(-c3cccc(F)c3C(F)(F)F)cc12]

M+1 calculated for C₂₅H₂₉F₄N₂O₂S 497.19, found 496.97.

### Example 243: N-(2-(5-(2-chloro-3-fluorophenyl)-3-neopentyl-1H-indol-1-yl)ethyl)cyclopropane sulfonamide (228)

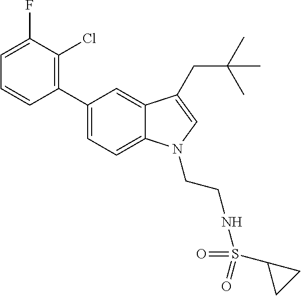 [SMILES: CC(C)(C)Cc1cn(CCNS(=O)(=O)C2CC2)c2ccc(-c3cccc(F)c3Cl)cc12]

M+1 calculated for C₂₄H₂₉FClN₂O₂S 463.16, found 462.88.

### Example 244: N-(2-(5-(4-amino-2-chloro-3-fluorophenyl)-3-neopentyl-1H-indol-1-yl)ethyl) cyclo-propanesulfonamide (229)

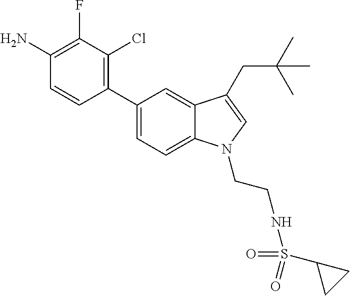 [SMILES: CC(C)(C)Cc1cn(CCNS(=O)(=O)C2CC2)c2ccc(-c3ccc(N)c(F)c3Cl)cc12]

M+1 calculated for C₂₄H₃₀ClFN₃O₂S 478.17, found 477.99.

### Example 245: N-(2-(5-(4-fluoro-2-(trifluoromethyl)phenyl)-3-neopentyl-1H-indol-1-yl)ethyl) cyclopropanesulfonamide (301)

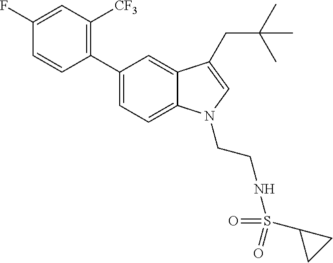 [SMILES: CC(C)(C)Cc1cn(CCNS(=O)(=O)C2CC2)c2ccc(-c3ccc(F)cc3C(F)(F)F)cc12]

M+1 calculated for C₂₅H₂₉F₄N₂O₂S 497.19, found 496.87.

### Example 246: N-(2-(3-neopentyl-5-(o-tolyl)-1H-indol-1-yl)ethyl)cyclopropanesulfonamide (302)

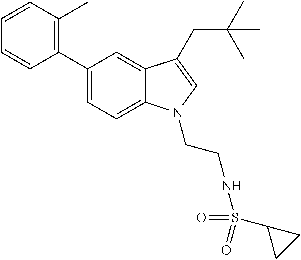 [SMILES: Cc1ccccc1-c1ccc2c(c1)c(CC(C)(C)C)cn2CCNS(=O)(=O)C1CC1]

M+1 calculated for C₂₅H₃₃N₂O₂S 425.23, found 424.89.

### Example 247: N-(2-(5-(2-methoxyphenyl)-3-neopentyl-1H-indol-1-yl)ethyl)cyclopropane-sulfonamide (303)

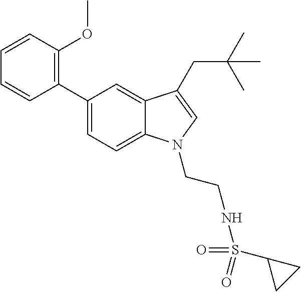 [SMILES: COc1ccccc1-c1ccc2c(c1)c(CC(C)(C)C)cn2CCNS(=O)(=O)C1CC1]

M+1 calculated for C₂₅H₃₃N₂O₃S 441.22, found 440.89.

### Example 248: N-(2-(3-neopentyl-5-(pyridin-2-yl)-1H-indol-1-yl)ethyl)cyclopropanesulfonamide (304)

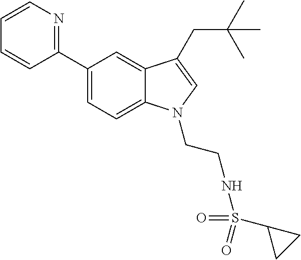 [SMILES: CC(C)(C)Cc1cn(CCNS(=O)(=O)C2CC2)c2ccc(-c3ccccn3)cc12]

M+1 calculated for C₂₃H₃₀N₃O₂S 412.21, found 412.79.

### Example 249: N-(2-(3-neopentyl-5-(pyridin-3-yl)-1H-indol-1-yl)ethyl)cyclopropanesulfonamide (305)

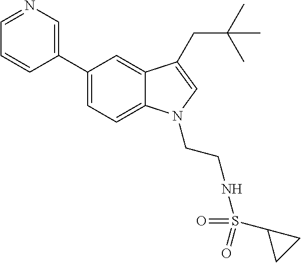 [SMILES: CC(C)(C)Cc1cn(CCNS(=O)(=O)C2CC2)c2ccc(-c3cccnc3)cc12]

M+1 calculated for C₂₃H₃₀N₃O₂S 412.21, found 411.99.

### Example 250: N-(2-(5-(2,4-bis(trifluoromethyl)phenyl)-3-neopentyl-1H-indol-1-yl)ethyl)cyclo-propanesulfonamide (299)

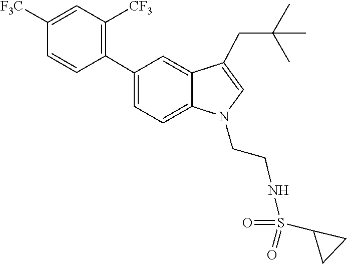 [SMILES: CC(C)(C)Cc1cn(CCNS(=O)(=O)C2CC2)c2ccc(-c3ccc(C(F)(F)F)cc3C(F)(F)F)cc12]

M+1 calculated for C₂₆H₂₉F₆N₂O₂S 547.19, found 546.84.

### Example 251: N-(2-(5-(2-chloro-4-(trifluoromethyl)phenyl)-3-neopentyl-1H-indol-1-yl)ethyl) cyclopropanesulfonamide (300)

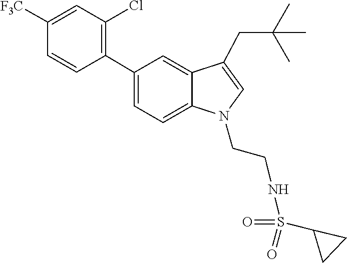 [SMILES: CC(C)(C)Cc1cn(CCNS(=O)(=O)C2CC2)c2ccc(-c3ccc(C(F)(F)F)cc3Cl)cc12]

M+1 calculated for C₂₅H₂₉ClF₃N₂O₂S 513.16, found 512.81.

### Example 252: N-(2-(5-methyl-3-neopentyl-1H-indol-1-yl)ethyl)cyclopropanesulfonamide (296)

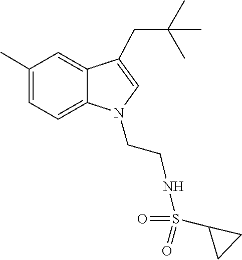 [SMILES: Cc1ccc2c(c1)c(CC(C)(C)C)cn2CCNS(=O)(=O)C1CC1]

M+1 calculated for C₁₉H₂₉N₂O₂S 349.19, found 348.81.

### Example 253: N-(2-(5-(2,6-dichloro-4-fluorophenyl)-3-neopentyl-1H-indol-1-yl)ethyl) cyclo-propane sulfonamide (297)

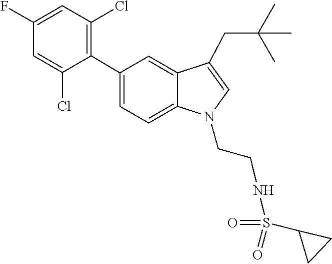 [SMILES: CC(C)(C)Cc1cn(CCNS(=O)(=O)C2CC2)c2ccc(-c3c(Cl)cc(F)cc3Cl)cc12]

M+1 calculated for C₂₄H₂₈Cl₂FN₂O₂S 497.12, found: 497.70.

### Examples 254 and 255

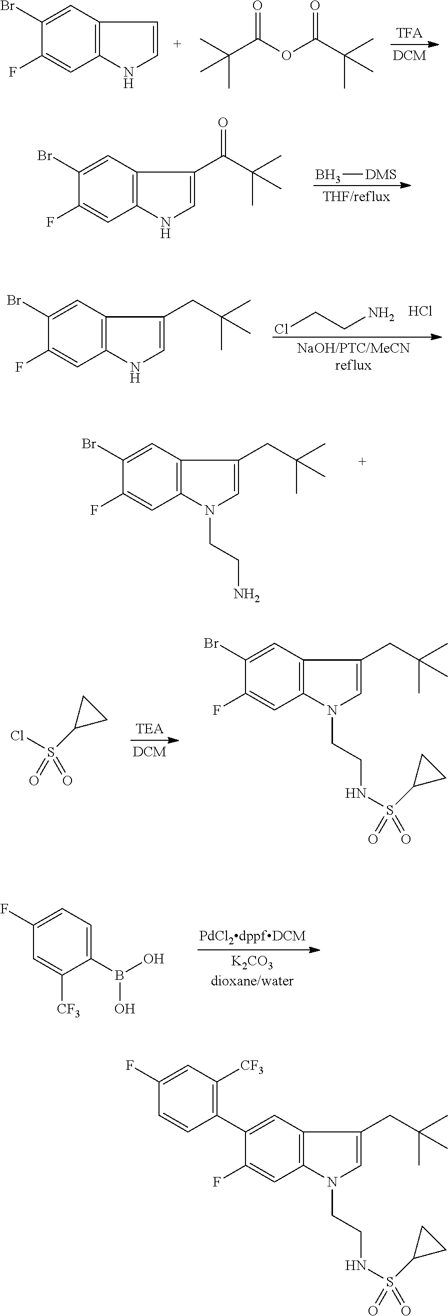

### 2-(5-bromo-6-fluoro-3-neopentyl-1H-indol-1-yl)ethan-1-amine

Add powdered NaOH (100 mg, 2.5 mmol) and tetrabutylammonium hydrogensulfate (30 mg, 0.11 mmol) to a solution of 5-bromo-6-fluoro-3-neopentyl-1H-indole (287 g, 1.0 mmol) in dry CH₃CN (10 mL). Stir the mixture at 25° C. for 30 minutes. Add 2-chloroethylamine hydrochloride (140 g, 1.2 mmol) to the mixture. Reflux the mixture for 24 hours. Filter off the inorganic solid. Wash the mixture with CH₂Cl₂. Dry the combined filtrate over anhydrous K₂CO₃. Concentrate the mixture. And the residue was purified by combi-flash with hexane/EtOAc (1:1) as an eluent to obtain 3-50-3 75 mg. M+1 calcd. C₁₅H₂₁BrFN₂ for 327.07, found 326.78.

### Example 254: N-(2-(5-bromo-6-fluoro-3-neopentyl-1H-indol-1-yl)ethyl)cyclopropanesulfonamide (293)

To a mixture of 2-(5-bromo-6-fluoro-3-neopentyl-1H-indol-1-yl)ethan-1-amine 75 mg (0.24 mmol) in DCM (1 mL) was added triethylamine 48 uL (0.36 mmol) and cyclopropanesulfonyl chloride 48 mg (0.3 mmol) at 0° C. The resulting mixture was stirred at room temperature for 12h until the starting material was consumed. The product was purified by prep-HPLC. M+1 calculated for C₁₈H₂₅BrFN₂O₂S 431.07, found 430.70.

### Example 255: 2-((4-fluoro-2-(trifluoromethyl)phenyl)-6-fluoro-3-neopentyl-1H-indol-1-yl)ethan-1-amine (294)

To a mixture of N-(2-(5-bromo-6-fluoro-3-neopentyl-1H-indol-1-yl)ethyl)cyclopropane sulfonamide 22 mg (0.05 mmol), (4-fluoro-2-(trifluoromethyl)phenyl)boronic acid 16 mg (0.08 mmol) and potassium carbonate 14 mg (0.01 mmol) in dioxane and water (1 mL, v/v=1:1) was added PdCl₂-dppf-DCM (2 mg). The mixture was heated at 100° C. for 1 hour. The dark solution was directly purified by prep-HPLC to obtain 03-51-3. M+1 calcd. for C₂₅H₂₈F₅N₂O₂S 515.18, found 514.81.

### Example 256: N-(2-((4-fluoro-2-(trifluoromethyl)phenyl)-4-fluoro-3-neopentyl-1H-indol-1-yl)ethyl) cyclopropanesulfonamide (295)

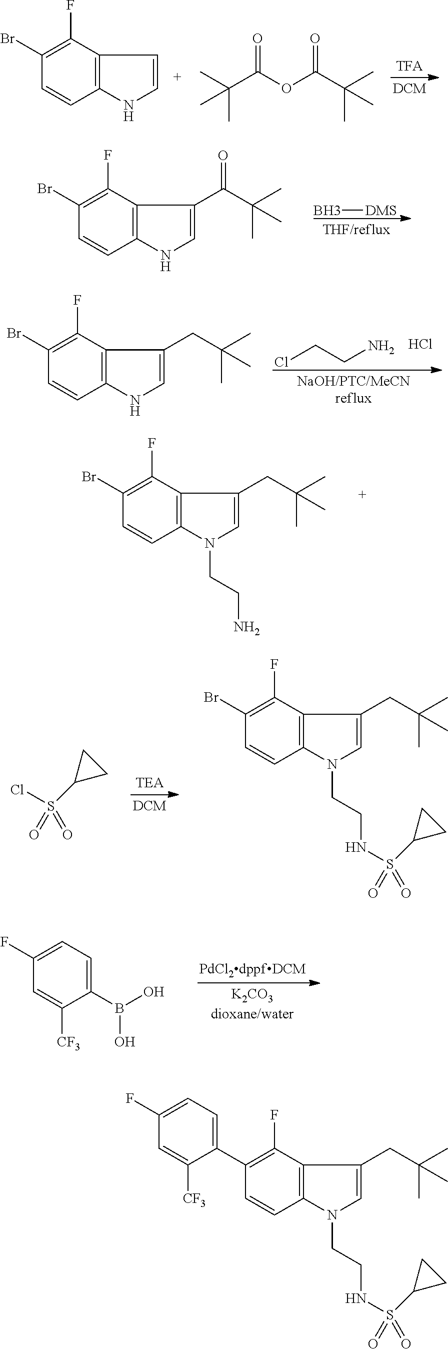

### 1-(5-bromo-4-fluoro-1H-indol-3-yl)-2,2-dimethylpropan-1-one

To a solution of 5-bromo4-fluoro-1H-indole 430 mg (2 mmol) in TFA (2 mL) and DCM (10 mL) was added pivalic anhydride 750 mg (4 mmol). The reaction mixture was stirred at room temperature overnight. The solvent was removed under reduced pressure and the residue was purified by combi-flash to obtain the title compound 450 mg, yield 75%. M+1 calculated for C₁₃H₁₄BrFNO 298.02, found 297.72.

### 5-bromo-4-fluoro-3-neopentyl-1H-indole

To a solution of 1-(5-bromo-4-fluoro-1H-indol-3-yl)-2,2-dimethylpropan-1-one 450 mg (1.5 mmol) in THF (20 mL) was added borane dimethyl sulfide complex 380 mg (5 mmol). The reaction mixture was stirred at reflux for 24 hours, and then cooled to 0° C. MeOH (20 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the title compound as an oil 290 mg, yield 68%. M+1 calculated. C₁₃H₁₆BrFN 283.04, found 282.75.

### 2-(5-bromo-4-fluoro-3-neopentyl-1H-indol-1-yl)ethan-1-amine

Powdered NaOH (100 mg, 2.5 mmol) and tetrabutylammonium hydrogensulfate (30 mg, 0.11 mmol) were added to a solution of 5-bromo-4-fluoro-3-neopentyl-1H-indole (290 g, 1.0 mmol) in dry CH₃CN (10 mL). After stirring at 25° C. for 30 minutes, 2-chloroethylamine hydrochloride (140 mg, 1.2 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the title compound 80 mg. M+1 calculated C₁₅H₂₁BrFN₂ for 327.07, found 326.87.

### N-(2-(5-bromo-4-fluoro-3-neopentyl-1H-indol-1-yl)ethyl)cyclopropanesulfonamide

To a solution of 2-(5-bromo-4-fluoro-3-neopentyl-1H-indol-1-yl)ethan-1-amine 65 mg (0.2 mmol) in DCM (1 mL) was added triethylamine 40 uL (0.32 mmol) and cyclopropanesulfonyl chloride 40 mg (0.3 mmol) at 0° C. The reaction mixture was stirred at room temperature for 12h until the starting material was consumed. The reaction mixture was directly purified by prep-HPLC. M+1 calculated C₁₈H₂₅BrFN₂O₂S 431.07, found 430.87.

### N-(2-((4-fluoro-2-(trifluoromethyl)phenyl)-4-fluoro-3-neopentyl-1H-indol-1-yl)ethyl) cyclopropanesulfonamide (295)

To a mixture of N-(2-(5-bromo-4-fluoro-3-neopentyl-1H-indol-1-yl)ethyl)cyclopropane sulfonamide 22 mg (0.05 mmol), (4-fluoro-2-(trifluoromethyl)phenyl)boronic acid 16 mg (0.08 mmol) and potassium carbonate 14 mg (0.01 mmol) in dioxane and water (1 mL, v/v=1:1) was added PdCl₂-dppf-DCM (2 mg). The mixture was heated at 100° C. for 1 hour. The dark solution was directly purified by prep-HPLC. M+1 calculated for C₂₅H₂₈F₅N₂O₂S 515.18, found 514.87.

### Example 257: N-(2-(3-neopentyl-5-(trifluoromethyl)-1H-indol-1-yl)ethyl)cyclopropanesulfonamide (292)

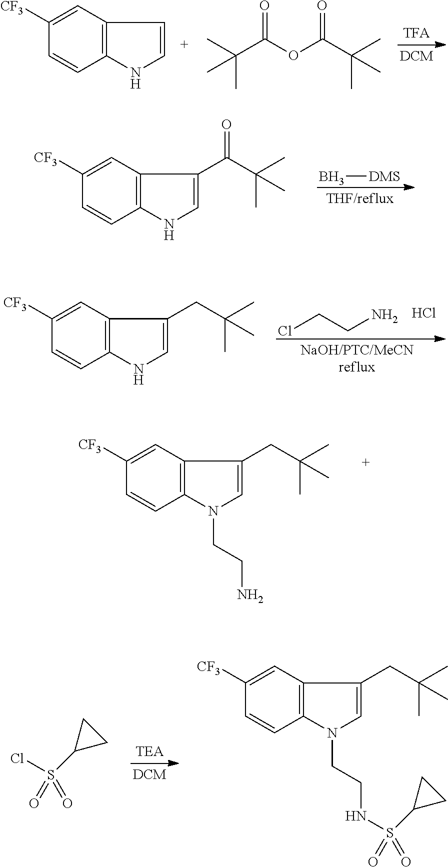

### 2,2-dimethyl-1-(5-(trifluoromethyl)-1H-indol-3-yl)propan-1-one

To a mixture of 5-(trifluoromethyl)-1H-indole 370 mg (2 mmol) in TFA (2 mL) and DCM (10 mL) was added pivalic anhydride 750 mg (4 mmol). The resulting mixture was stirred at room temperature overnight. The solvent was removed under reduced pressure and the residue was purified by combi-flash to afford 430 mg product, yield 80%. M+1 calculated for C₁₄H₁₅F₃NO 270.10, found 270.02.

### 3-neopentyl-5-(trifluoromethyl)-1H-indole

To a mixture of 2,2-dimethyl-1-(5-(trifluoromethyl)-1H-indol-3-yl)propan-1-one 430 mg (1.6 mmol) in THF (20 mL) was added borane dimethyl sulfide complex 380 mg (5 mmol). The reaction mixture was stirred at reflux for 12 hours, and then cooled to 0° C. MeOH (20 mL) was added, and the reaction mixture was concentrated in vacuo. The remaining residue with diluted with water (50 mL) and CH₂Cl₂ and the layers were separated. The aqueous layer was extracted with CH₂Cl₂ (3×50 mL). The combined organics were washed with water (2×50 mL), dried (Na₂SO₄) and filtered. The filtrate was concentrated under reduced pressure and the crude residue was purified by combi-flash to obtain the title compound 285 mg, yield 70%. ¹HNMR (CDCl₃, 400 Hz) 0.96 (s, 9H), 2.65 (s, 2H), 7.07 (s, 1H), 7.23 (s, 1H), 7.28 (t, J=9.5 Hz), 7.88 (s, 1H), 8.20 (br, 1H).

### 2-(3-neopentyl-5-(trifluoromethyl)-1H-indol-1-yl)ethan-1-amine

Powdered NaOH (90 mg, 2.2 mmol) and tetrabutylammonium hydrogensulfate (30 mg, 0.11 mmol) were added to a solution of 3-neopentyl-5-(trifluoromethyl)-1H-indole (280 g, 1.1 mmol) in dry CH₃CN (10 mL). After stirring at 25° C. for 30 minutes, 2-chloroethylamine hydrochloride (140 mg, 1.2 mmol) was added. The mixture was warmed to reflux for 24 hours, cooled, and the inorganic solids were filtered and washed with CH₂Cl₂. The filtrate was dried over anhydrous K₂CO₃ and then filtered. The filtrate was concentrated and the crude residue was purified by combi-flash with hexane/EtOAc (1:1) as an eluent to afford the title compound (45 mg). M+1 calculated C₁₆H₂₂F₃N₂ for 299.17, found 298.87.

### N-(2-(3-neopentyl-5-(trifluoromethyl)-1H-indol-1-yl)ethyl)cyclopropanesulfonamide (292)

To a solution of 2-(3-neopentyl-5-(trifluoromethyl)-1H-indol-1-yl)ethan-1-amine 25 mg (0.08 mmol) in DCM (1 mL) was added triethylamine 20 uL (0.16 mmol) and cyclopropanesulfonyl chloride 17 mg (0.12 mmol) at 0° C. The reaction mixture was stirred at room temperature for 12h until the starting material was consumed. The reaction mixture was directly purified by prep-HPLC. M+1 calculated C₁₉H₂₆F₃N₂O₂S 403.17, found 402.78.

### Example 258: (S)—N-(2,2,2-trifluoro-1-(5-fluoro-1-neopentyl-6-(2-(trifluoromethyl)phenyl)-1H-indol-3-yl)ethyl)azetidine-1-sulfonamide (346)

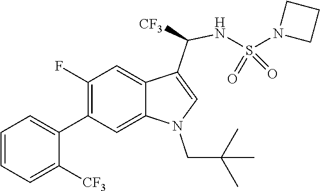 [SMILES: CC(C)(C)Cn1cc([C@H](NS(=O)(=O)N2CCC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and azetidine-1-sulfonyl chloride. ESI-MS (m/z): 565.67 [M+1]⁺.

### Example 259: (S)—N-(2,2,2-trifluoro-1-(5-fluoro-1-neopentyl-6-(2-(trifluoromethyl)phenyl)-1H-indol-3-yl)ethyl)pyrrolidine-1-sulfonamide (347)

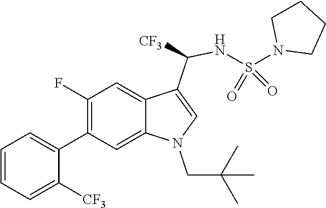 [SMILES: CC(C)(C)Cn1cc([C@H](NS(=O)(=O)N2CCCC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and pyrrolidine-1-sulfonyl chloride. ESI-MS (m/z): 579.66 [M+1]⁺.

### Example 260: (S)—N-(2,2,2-trifluoro-1-(5-fluoro-1-neopentyl-6-(2-(trifluoromethyl)phenyl)-1H-indol-3-yl)ethyl)propane-2-sulfonamide (348)

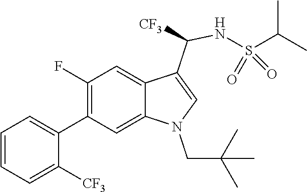 [SMILES: CC(C)S(=O)(=O)N[C@@H](c1cn(CC(C)(C)C)c2cc(-c3ccccc3C(F)(F)F)c(F)cc12)C(F)(F)F]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and propane-2-sulfonyl chloride. ¹HNMR (CDCl₃, 400 MHz) δ 7.79 (d, J=4.0 Hz, 1H), 7.60-7.50 (m, 2H), 7.46-7.25 (m, 3H), 7.22 (d, J=8.0 Hz, 1H), 5.22-5.17 (m, 1H), 4.63 (s, 1H). 4.41-4.32 (m, 1H), 3.95-3.85 (m, 2H), 1.86-1.75 (m, 6H), 0.98 (s, 9H)

### Example 261: (S)—N-(1-(1-(cyclopropylmethyl)-5-fluoro-6-(2-(trifluoromethyl)phenyl)-1H-indol-3-yl)-2,2,2-trifluoroethyl)cyclopropanesulfonamide (349)

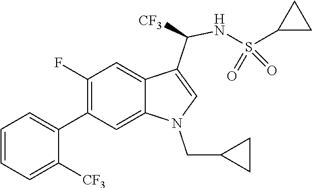 [SMILES: O=S(=O)(N[C@@H](c1cn(CC2CC2)c2cc(-c3ccccc3C(F)(F)F)c(F)cc12)C(F)(F)F)C1CC1]

The title compound was prepared following the same general protocol as described for Step 5, Example 116, using (S)-1-(1-(cyclopropylmethyl)-5-fluoro-6-(2-(trifluoromethyl)phenyl)-1H-indol-3-yl)-2,2,2-trifluoroethan-1-amine and cyclopropanesulfonyl chloride. (S)-1-(1-(cyclopropylmethyl)-5-fluoro-6-(2-(trifluoromethyl)phenyl)-1H-indol-3-yl)-2,2,2-trifluoroethan-1-amine was made following the general protocols as described in Example 133 starting with 6-bromo-1-(cyclopropylmethyl)-5-fluoro-1H-indole. ¹HNMR (CDCl₃, 400 MHz) δ 7.79 (d, J=8.0 Hz, 1H), 7.61-7.43 (m, 3H), 7.40-7.36 (m, 2H), 7.26-7.21 (m, 1H), 5.38 (q, J=8.0 Hz, 1H), 4.83-4.78 (m, 1H), 3.94 (d, J=8.0 Hz, 2H), 2.51-2.38 (m, 1H), 1.29-1.06 (m, 3H), 0.99-0.86 (m, 2H), 0.67-0.65 (m, 2H), 0.37-0.33 (m, 2H)

### Example 262: (S)—N-(2,2,2-trifluoro-1-(5-fluoro-1-neopentyl-6-phenyl-1H-indol-3-yl)ethyl)cyclopropanesulfonamide (350)

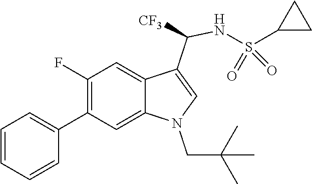 [SMILES: CC(C)(C)Cn1cc([C@H](NS(=O)(=O)C2CC2)C(F)(F)F)c2cc(F)c(-c3ccccc3)cc21]

The title compound was prepared following the same general protocol as described for Step 5, Example 116, using (S)-2,2,2-trifluoro-1-(5-fluoro-1-neopentyl-6-phenyl-1H-indol-3-yl)ethan-1-amine and cyclopropanesulfonyl chloride. (S)-2,2,2-trifluoro-1-(5-fluoro-1-neopentyl-6-phenyl-1H-indol-3-yl)ethan-1-amine was made following the general protocols as described in Example 133 starting with 6-bromo-5-fluoro-1-neopentyl-1H-indole. ¹HNMR (CDCl₃, 400 MHz) δ 7.57-7.55 (m, 2H), 7.49-7.44 (m, 3H), 7.40-7.33 (m, 2H), 7.25 (broad s, 1H), 5.34 (q, J=8.0 Hz, 1H), 5.11 (d, J=8.0 Hz, 1H), 3.89 (s, 2H), 2.48-2.42 (m, 1H), 1.27-1.10 (m, 2H), 1.00-0.90 (m, 11H)

### Example 263: N-((1S)-2,2,2-trifluoro-1-(5-fluoro-6-(2-fluoro-6-(trifluoromethyl)phenyl)-1-neopentyl-1H-indol-3-yl)ethyl)cyclopropanesulfonamide (351)

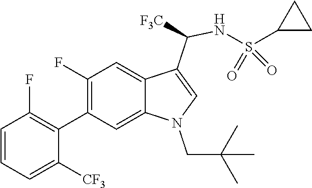 [SMILES: CC(C)(C)Cn1cc([C@H](NS(=O)(=O)C2CC2)C(F)(F)F)c2cc(F)c(-c3c(F)cccc3C(F)(F)F)cc21]

The title compound was prepared following the same general protocol as described for Step 5, Example 116, using (1S)-2,2,2-trifluoro-1-(5-fluoro-6-(2-fluoro-6-(trifluoromethyl)phenyl)-1-neopentyl-1H-indol-3-yl)ethan-1-amine and cyclopropanesulfonyl chloride. (1S)-2,2,2-trifluoro-1-(5-fluoro-6-(2-fluoro-6-(trifluoromethyl)phenyl)-1-neopentyl-1H-indol-3-yl)ethan-1-amine was made following the general protocols as described in Example 133 starting with 6-bromo-5-fluoro-1-neopentyl-1H-indole. ¹HNMR (CDCl₃, 400 MHz) δ 7.60 (dd, J=8.0 Hz, 4.0 Hz, 1H), 7.54-7.47 (m, 2H), 7.38-7.31 (m, 1H), 7.27 (d, J=8.0 Hz, 1H), 7.20 (dd, J=8.0 Hz, 4.0 Hz, 1H), 5.37 (q, J=8.0 Hz, 1H), 5.22-5.14 (m, 1H), 3.91-3.71 (m, 2H), 2.49-2.40 (m, 1H), 1.28-1.24 (m, 2H), 0.97-0.93 (m, 11H).

### Example 264: (S)—N-(2,2,2-trifluoro-1-(7-methyl-1-neopentyl-6-(2-(trifluoromethyl)phenyl)-1H-indol-3-yl)ethyl)cyclopropanesulfonamide (353)

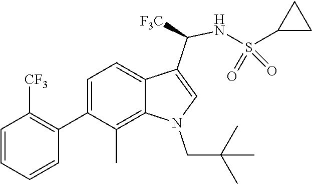 [SMILES: Cc1c(-c2ccccc2C(F)(F)F)ccc2c([C@H](NS(=O)(=O)C3CC3)C(F)(F)F)cn(CC(C)(C)C)c12]

The title compound was prepared following the same general protocol as described for Step 5, Example 116, using (S)-2,2,2-trifluoro-1-(7-methyl-1-neopentyl-6-(2-(trifluoromethyl)phenyl)-1H-indol-3-yl)ethan-1-amine and cyclopropanesulfonyl chloride. (S)-2,2,2-trifluoro-1-(7-methyl-1-neopentyl-6-(2-(trifluoromethyl)phenyl)-1H-indol-3-yl)ethan-1-amine was made following the general protocols as described in Example 133 starting with 6-bromo-7-methyl-1-neopentyl-1H-indole. ¹HNMR (CDCl₃, 400 MHz) δ 7.75 (d, J=8.0 Hz, 1H), 7.58-7.53 (m, 2H), 7.48-7.7.46 (m, 1H), 7.25 (d, J=8.0 Hz, 1H), 7.20 (s, 4.0 Hz, 1H), 6.96 (d, J=8.0 Hz, 1H), 5.45-5.41 (m, 1H), 5.12-4.98 (m, 1H), 4.33-4.08 (m, 2H), 2.44-2.40 (m, 1H), 1.28-1.22 (m, 2H), 0.91-0.85 (m, 11H).

### Example 265: (S)—N-(2,2,2-trifluoro-1-(5-fluoro-6-(4-fluoro-2-(trifluoromethyl)phenyl)-1-neopentyl-1H-indol-3-yl)ethyl)cyclopropanesulfonamide (355)

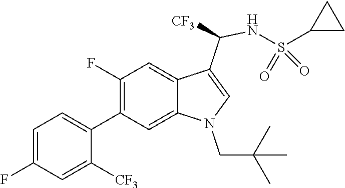 [SMILES: CC(C)(C)Cn1cc([C@H](NS(=O)(=O)C2CC2)C(F)(F)F)c2cc(F)c(-c3ccc(F)cc3C(F)(F)F)cc21]

The title compound was prepared following the same general protocol as described for Step 5, Example 116, using (S)-2,2,2-trifluoro-1-(5-fluoro-6-(4-fluoro-2-(trifluoromethyl)phenyl)-1-neopentyl-1H-indol-3-yl)ethan-1-amine and cyclopropanesulfonyl chloride. (S)-2,2,2-trifluoro-1-(5-fluoro-6-(4-fluoro-2-(trifluoromethyl)phenyl)-1-neopentyl-1H-indol-3-yl)ethan-1-amine was made following the general protocols as described in Example 133 starting with 6-bromo-5-fluoro-1-neopentyl-1H-indole. ¹HNMR (CDCl₃, 400 MHz) δ 7.52-7.45 (m, 2H), 7.37-7.25 (m, 3H), 7.20 (d, J=4.0 Hz, 1H), 5.38 (q, J=8.0 Hz, 1H), 4.84-4.78 (m, 1H), 3.95-3.85 (m, 2H), 2.47-2.40 (m, 1H), 1.27-1.10 (m, 2H), 1.00-0.90 (m, 11H).

### Example 266: (S)—N-(2,2,2-trifluoro-1-(1-neopentyl-5-(trifluoromethyl)-6-(2-(trifluoromethyl)phenyl)-1H-indol-3-yl)ethyl)cyclopropanesulfonamide (357)

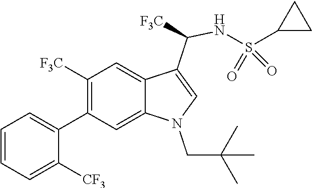 [SMILES: CC(C)(C)Cn1cc([C@H](NS(=O)(=O)C2CC2)C(F)(F)F)c2cc(C(F)(F)F)c(-c3ccccc3C(F)(F)F)cc21]

The title compound was prepared following the same general protocol as described for Step 5, Example 116, using (S)-2,2,2-trifluoro-1-(1-neopentyl-5-(trifluoromethyl)-6-(2-(trifluoromethyl)phenyl)-1H-indol-3-yl)ethan-1-amine and cyclopropanesulfonyl chloride. (S)-2,2,2-trifluoro-1-(1-neopentyl-5-(trifluoromethyl)-6-(2-(trifluoromethyl)phenyl)-1H-indol-3-yl)ethan-1-amine was made following the general protocols as described in Example 133 starting with 6-bromo-1-neopentyl-5-(trifluoromethyl)-1H-indole. ¹HNMR (CDCl₃, 400 MHz) δ 8.10 (s, 1H), 7.67 (s, 1H), 7.25 (s, 1H), 5.38 (q, J=8.0 Hz, 1H), 5.05 (d, J=8.0 Hz, 1H), 3.83 (s, 2H), 2.47-2.41 (m, 1H), 1.31-1.10 (m, 2H), 0.99-0.83 (m, 11H).

### Example 267: (S)—N-(2,2,2-trifluoro-1-(5-fluoro-6-(3-fluoro-2-(trifluoromethyl)phenyl)-1-neopentyl-1H-indol-3-yl)ethyl)cyclopropanesulfonamide (360)

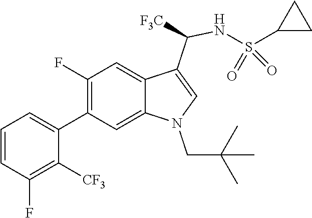 [SMILES: CC(C)(C)Cn1cc([C@H](NS(=O)(=O)C2CC2)C(F)(F)F)c2cc(F)c(-c3cccc(F)c3C(F)(F)F)cc21]

The title compound was prepared following the same general protocol as described for Step 5, Example 116, using (S)-2,2,2-trifluoro-1-(5-fluoro-6-(3-fluoro-2-(trifluoromethyl)phenyl)-1-neopentyl-1H-indol-3-yl)ethan-1-amine and cyclopropanesulfonyl chloride. (S)-2,2,2-trifluoro-1-(5-fluoro-6-(3-fluoro-2-(trifluoromethyl)phenyl)-1-neopentyl-1H-indol-3-yl)ethan-1-amine was made following the general protocols as described in Example 133 starting with 6-bromo-5-fluoro-1-neopentyl-1H-indole. ¹HNMR (CDCl₃, 400 MHz) δ 7.57-7.51 (m, 1H), 7.45 (t, J=8.0 Hz, 1H), 7.27-7.17 (m, 2H), 7.18 (d, J=8.0 Hz, 1H), 7.11 (d, J=8.0 Hz, 1H), 5.34 (q, J=8.0 Hz, 1H), 5.10-4.97 (m, 1H), 3.87-3.85 (m, 2H), 2.50-2.39 (m, 1H), 1.43-1.21 (m, 2H), 0.97-0.82 (m, 11H).

### Example 268: (S)—N-(1-(6-(3-chloropyridin-4-yl)-5-fluoro-1-neopentyl-1H-indol-3-yl)-2,2,2-trifluoroethyl)cyclopropanesulfonamide (361)

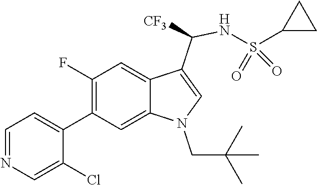 [SMILES: CC(C)(C)Cn1cc([C@H](NS(=O)(=O)C2CC2)C(F)(F)F)c2cc(F)c(-c3ccncc3Cl)cc21]

The title compound was prepared following the same general protocol as described for Step 5, Example 116, using (S)-1-(6-(3-chloropyridin-4-yl)-5-fluoro-1-neopentyl-1H-indol-3-yl)-2,2,2-trifluoroethan-1-amine and cyclopropanesulfonyl chloride. (S)-1-(6-(3-chloropyridin-4-yl)-5-fluoro-1-neopentyl-1H-indol-3-yl)-2,2,2-trifluoroethan-1-amine was made following the general protocols as described in Example 133 starting with 6-bromo-5-fluoro-1-neopentyl-1H-indole. ESI-MS (m/z): 518.01 [M+1]⁺.

Compound Synthesis Examples: Part 2. Example numbers correspond to compound numbers in this part, and they are presented with the prefix “B-”.

General Experimental Conditions

All evaporations were carried out in vacuo with a rotary evaporator. Analytical samples were dried in vacuo (5 mmHg) at rt. Thin layer chromatography (TLC) was performed on silica gel plates, spots were visualized by UV light (254 and 365 nm). Purification by column and flash chromatography was carried out using silica gel (200-300 mesh). All NMR spectra were recorded on a Bruker 400 (400 MHz) spectrometer. ¹H chemical shifts are reported in δ values in ppm with the deuterated solvent as the internal standard. NMR signals are reported as follows: chemical shift, multiplicity (s=singlet, d=doublet, t=triplet, q=quartet, br=broad, m=multiplet), coupling constant (Hz), integration. LCMS spectra were obtained on an Agilent 1200 series with a 6120 mass spectrometer using electrospray ionization.

Methods

LCMS Method A

Column: Waters X-Bridge C18 (50 mm*4.6 mm*3.5 m); Column Temperature: 40° C.; Flow Rate: 2.0 mL/min; Mobile Phase: from 95% [water+0.1% NH₄OH] and 5% [CH₃CN] to 0% [water+0% NH₄OH] and 100% [CH₃CN] in 1.6 min, then under this condition for 1.4 min, finally changed to 95% [water+0% NH₄OH] and 5% [CH₃CN] in 0.1 min and under this condition for 0.7 min.

LCMS Method B

Column: Waters X-Bridge C18 (50 mm*4.6 mm*3.5 m); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 3.0 min, then under this condition for 1.0 min, finally changed to 95% [water+10 mM NH₄HCO₃] and 5% [CH₃CN] in 0.1 min and under this condition for 0.7 min.

### Example B-1: N-{2-[1-(2-methylpropyl)-6-(pyridin-4-yl)-1H-indol-3-yl]ethyl}propane-2-sulfonamide (1)

### Step 1. 6-bromo-1-isobutyl-1H-indole

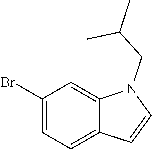

To a solution of 6-bromo-1H-indole (10.0 g, 51.01 mmol) and 1-bromo-2-methylpropane (10.48 g, 76.51 mmol) in DMF (100 mL), were added Cs₂CO₃ (24.93 g, 76.51 mmol) and KI (846.76 mg, 5.10 mmol). The mixture was stirred at 80° C. overnight, and then cooled to room temperature. Water (300 mL) and EA (200 mL) was added. The organic layer was separated. The aqueous layer was extracted with EA (2×100 mL). The combined organic layer was washed with brine (2×100 mL), dried over anhydrous Na₂SO₄ and evaporated to give crude product which was purified by silica gel column chromatography to give the product as a yellow oil (8.5 g, yield: 66%).

### Step 2. 1-isobutyl-6-(pyridin-4-yl)-1H-indole

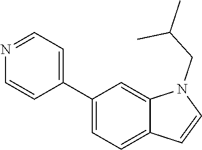 [SMILES: CC(C)Cn1ccc2ccc(-c3ccncc3)cc21]

To a solution of 6-bromo-1-isobutyl-1H-indole (3 g, 12.0 mmol), Na₂CO₃ (2.53 g, 24 mmol) and 4-(4,4,5,5-tetramethyl-1,3,2-dioxaborolan-2-yl)pyridine (2.69 g, 13.4 mol) in dioxane (50 mL) and water (5 mL), was added Pd(dppf)Cl₂ (0.30 g) under N₂ atmosphere. The reaction mixture was heated to 90° C. and stirred for 2 h. The resulting reaction was concentrated under reduced pressure, and then water (100 mL) was added. The mixture was extracted with EA (2×100 mL). The combined organic layers were washed with brine (2×50 mL), dried over Na₂SO₄, filtered, and concentrated under reduced pressure. The residue was purified by silica gel column chromatography to give the product as a yellow solid (2.4 g, yield: 80%).

### Step 3. (Z)-1-isobutyl-3-(2-nitrovinyl)-6-(pyridin-4-yl)-1H-indole

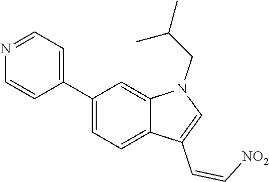 [SMILES: CC(C)Cn1cc(/C=C\[N+](=O)[O-])c2ccc(-c3ccncc3)cc21]

To a solution of 1-isobutyl-6-(pyridin-4-yl)-1H-indole (1.2 g, 4.8 mmol) and (E)-N,N-dimethyl-2-nitroethenamine (0.56 g, 4.8 mmol) in DCM (5.00 mL), was added TFA (0.30 g). The reaction mixture was stirred overnight. The solvent was removed under reduced pressure. The residue was dissolved in DCM (20.0 mL), washed with saturated NaHCO₃ (20.0 mL), dried over Na₂SO₄, filtered, and concentrated to give the product as a yellow oil (1.54 g, yield: 100%).

### Step 4. 2-(1-isobutyl-6-(pyridin-4-yl)-1H-indol-3-yl)ethanamine

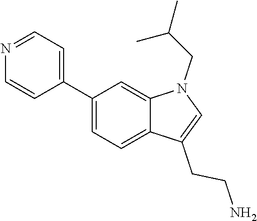 [SMILES: CC(C)Cn1cc(CCN)c2ccc(-c3ccncc3)cc21]

To a solution of (Z)-1-isobutyl-3-(2-nitrovinyl)-6-(pyridin-4-yl)-1H-indole (3.08 g, 9.6 mmol) in THF (25.0 mL) in an ice-water bath, was added LAH (0.69 g, 18.2 mmol) in portions. The reaction mixture was allowed to warm to room temperature and stirred for 5 h. Water (1.4 mL) and 10% NaOH (1.4 mL) were added. The solid was for filtered off. The filtrate was evaporated to give the product as a yellow solid (700 mg, yield: 24%).

### Step 5. N-{2-[1-(2-methylpropyl)-6-(pyridin-4-yl)-1H-indol-3-yl]ethyl}propane-2-sulfonamide

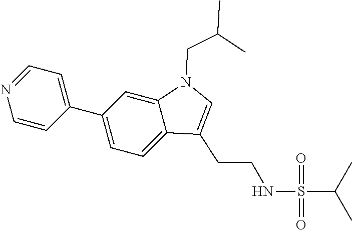 [SMILES: CC(C)Cn1cc(CCNS(=O)(=O)C(C)C)c2ccc(-c3ccncc3)cc21]

To a solution of 2-(1-isobutyl-6-(pyridin-4-yl)-1H-indol-3-yl)ethanamine (80 mg, 0.27 mmol) and DIPEA (105 mg, 0.81 mmol) in DCM (5.0 mL) in an ice-water bath, was added propane-2-sulfonyl chloride (78.1 g, 0.55 mmol) dropwise. The reaction mixture was allowed to warm to room temperature and stirred overnight. The solvent was removed under reduced pressure. The residue was purified by prep-HPLC to give the product as a yellow solid (17 mg, yield: 16%). ¹H NMR (400 MHz, d₆-DMSO): δ 8.59-8.61 (m, 2H), 7.93 (d, J=0.8 Hz, 1H), 7.78-7.79 (m, 2H), 7.64 (d, J=8.0 Hz, 1H), 7.46-7.48 (m, 1H), 7.32 (s, 1H), 7.14 (t, J=5.6 Hz, 1H), 4.03 (d, J=7.2 Hz, 2H), 3.20-3.25 (m, 2H), 3.11-3.18 (m, 1H), 2.89 (t, J=7.8 Hz, 2H), 2.11-2.18 (m, 1H), 1.19 (d, J=6.8 Hz, 6H), 0.87 (d, J=6.8 Hz, 6H). LCMS (Method A) RT 2.17 min, calcd. for C₂₂H₂₉N₃O₂S 399.20 m/z, found 400.3 m/z [M+H]+.

### Example B-2: N-{2-[1-(2-methylpropyl)-6-(pyridin-4-yl)-1H-indol-3-yl]ethyl}pyrrolidine-1-sulfonamide

### Step 1. N-{2-[1-(2-methylpropyl)-6-(pyridin-4-yl)-1H-indol-3-yl]ethyl}pyrrolidine-1-sulfonamide

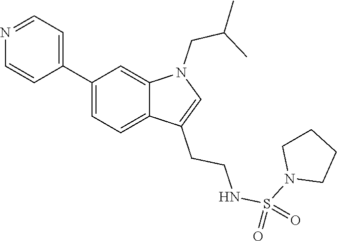 [SMILES: CC(C)Cn1cc(CCNS(=O)(=O)N2CCCC2)c2ccc(-c3ccncc3)cc21]

To a solution of 2-(1-isobutyl-6-(pyridin-4-yl)-1H-indol-3-yl)ethanamine (50 mg, 0.17 mmol) and DIPEA (66 mg, 0.51 mmol) in DCM (5.0 mL) in an ice-water bath, was added pyrrolidine-1-sulfonyl chloride (58 mg, 0.34 mmol) dropwise. The reaction mixture was allowed to warm to room temperature and stirred overnight. The solvent was removed under reduced pressure. The residue was purified by prep-HPLC to give the product as a yellow solid (8 mg, yield: 11%). ¹H NMR (400 MHz, d₆-DMSO): δ 8.59-8.61 (m, 2H), 7.93 (s, 1H), 7.78-7.79 (m, 2H), 7.64 (d, J=8.4 Hz, 1H), 7.46-7.48 (m, 1H), 7.32 (s, 1H), 7.21 (t, J=6.0 Hz, 1H), 4.03 (d, J=7.2 Hz, 2H), 3.17-3.22 (m, 2H), 3.07-3.11 (m, 4H), 2.89 (t, J=7.6 Hz, 2H), 2.11-2.17 (m, 1H), 1.71-1.78 (m, 4H), 0.87 (d, J=6.4 Hz, 6H). LCMS (Method A) RT 2.105 min, calcd. for C₂₃H₃₀N₄O₂S 426.21 m/z, found 427.4 m/z [M+H]⁺.

### Example B-3: N-{2-[1-(2-methylpropyl)-6-(pyridin-4-yl)-1H-indol-3-yl]ethyl}methanesulfonamide

### Step 1. N-{2-[1-(2-methylpropyl)-6-(pyridin-4-yl)-1H-indol-3-yl]ethyl}methanesulfonamide

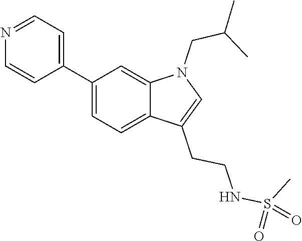 [SMILES: CC(C)Cn1cc(CCNS(C)(=O)=O)c2ccc(-c3ccncc3)cc21]

To a solution of 2-(1-isobutyl-6-(pyridin-4-yl)-1H-indol-3-yl)ethanamine (50 mg, 0.17 mmol) and DIPEA (66 mg, 0.51 mmol) in DCM (5.0 mL) in an ice-water bath, was added methanesulfonic anhydride (59 mg, 0.34 mmol) dropwise. The reaction mixture was allowed to warm to room temperature and stirred overnight. The solvent was removed under reduced pressure. The residue was purified by prep-HPLC to give the product as a yellow solid (4 mg, yield: 6%). ¹H NMR (400 MHz, d₆-DMSO): δ 8.59-8.61 (m, 2H), 7.93 (d, J=0.8 Hz, 1H), 7.78-7.80 (m, 2H), 7.65 (d, J=8.4 Hz, 1H), 7.46-7.49 (m, 1H), 7.33 (s, 1H), 7.13 (t, J=6.0 Hz, 1H), 4.03 (d, J=7.6 Hz, 2H), 3.20-3.25 (m, 2H), 2.91 (t, J=7.6 Hz, 2H), 2.86 (s, 3H), 2.08-2.20 (m, 1H), 0.87 (d, J=6.8 Hz, 6H). LCMS (Method A) RT 1.927 min, calcd. for C₂₀H₂₅N₃O₂S 371.17 m/z, found 372.4 m/z [M+H]+.

### Example B-5: dimethyl[methyl({2-[1-(2-methylpropyl)-6-(pyridin-4-yl)-1H-indol-3-yl]ethyl})sulfamoyl]amine

### Step 1. tert-butyl 2-(1-isobutyl-6-(pyridin-4-yl)-1H-indol-3-yl)ethylcarbamate

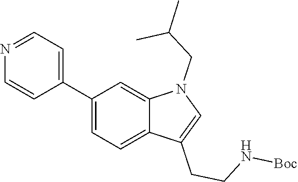 [SMILES: CC(C)Cn1cc(CCNC(=O)OC(C)(C)C)c2ccc(-c3ccncc3)cc21]

To a solution of 2-(1-isobutyl-6-(pyridin-4-yl)-1H-indol-3-yl)ethanamine (150 mg, 0.51 mmol) and DIPEA (197 mg, 1.53 mmol) in DCM (5.0 mL) in an ice-water bath, was added (Boc)₂O (134 mg, 0.61 mmol) dropwise. The reaction mixture was allowed to warm to room temperature and stirred for 2 h. The solvent was removed under reduced pressure. The residue was purified by prep-HPLC to give the product as a yellow oil (160 mg, yield: 80%).

### Step 2. 2-(1-isobutyl-6-(pyridin-4-yl)-1H-indol-3-yl)-N-methylethanamine

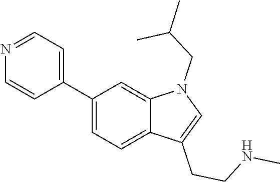 [SMILES: CNCCc1cn(CC(C)C)c2cc(-c3ccncc3)ccc12]

To a solution of tert-butyl 2-(1-isobutyl-6-(pyridin-4-yl)-1H-indol-3-yl)ethylcarbamate (140 mg, 0.36 mmol) in THF (5.0 mL) in an ice-water bath, was added LAH (68 mg, 1.80 mmol) in portions. The reaction mixture was refluxed overnight. And then cooled to room temperature, 10% aq. NaOH (0.3 mL) was added. The solid was filtered off. The filtrate was evaporated to give the product as a colorless oil (110 mg, yield: 100%).

### Step 3. dimethyl[methyl({2-[1-(2-methylpropyl)-6-(pyridin-4-yl)-1H-indol-3-yl]ethyl})sulfamoyl]amine

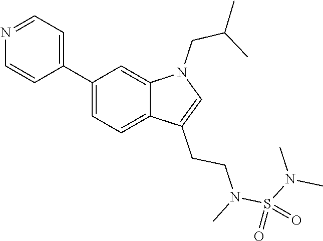 [SMILES: CC(C)Cn1cc(CCN(C)S(=O)(=O)N(C)C)c2ccc(-c3ccncc3)cc21]

To a solution of 2-(1-isobutyl-6-(pyridin-4-yl)-1H-indol-3-yl)-N-methylethanamine (120 mg, 0.39 mmol) and DIPEA (151 mg, 1.17 mmol) in DCM (5.0 mL) in an ice-water bath, was added dimethylsulfamoyl chloride (112 mg, 0.78 mmol) dropwise. The reaction mixture was allowed to warm to room temperature and stirred for 2 h. The solvent was removed under reduced pressure. The residue was purified by prep-HPLC to give the product as a yellow solid (45 mg, yield: 28%). ¹H NMR (400 MHz, d₆-DMSO): δ 8.59-8.61 (m, 2H), 7.93 (d, J=1.2 Hz, 1H), 7.77-7.80 (m, 2H), 7.68 (d, J=8.0 Hz, 1H), 7.46-7.49 (m, 1H), 7.33 (s, 1H), 4.03 (d, J=7.2 Hz, 2H), 3.39-3.42 (m, 2H), 2.96-3.00 (m, 2H), 2.84 (s, 3H), 2.66 (s, 6H), 2.11-2.18 (m, 1H), 0.86 (d, J=6.8 Hz, 6H). LCMS (Method A) RT 2.297 min, calcd. for C₂₂H₃₀N₄O₂S 414.56 m/z, found 415.4 m/z [M+H]+.

### Example B-6: dimethyl({2-[1-propyl-6-(pyridin-4-yl)-1H-indol-3-yl]ethyl}sulfamoyl)amine

### Step 1. 6-(pyridin-4-yl)-1H-indole

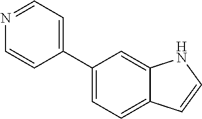 [SMILES: c1cc(-c2ccc3cc[nH]c3c2)ccn1]

To a stirred suspension of 6-bromo-1H-indole (10 g, 51.3 mmol), 4-(4,4,5,5-tetramethyl-1,3,2-dioxaborolan-2-yl)pyridine (10.5 g, 51.3 mmol) and Na₂CO₃ (8.2 g, 76.9 mmol) in dioxane/water (100 mL/10 mL), was added Pd(dppf)Cl₂ (7.5 g, 10.3 mmol). The resulting reaction mixture was stirred at 100° C. for 5 h and cooled down to room temperature at which time the solvent was removed. The residue was partitioned between water (100 mL) and EA (100 mL). The organic layer was separated, the aqueous layer was extracted with EA (2×50 mL), the combined organic layer was washed with brine (2×50 mL), dried over Na₂SO₄, filtered, and concentrated. The residue was purified by prep-HPLC to give the desired product (3.5 g, yield: 35%) as a white solid.

### Step 2. 2-oxo-2-(6-(pyridin-4-yl)-1H-indol-3-yl)acetamide

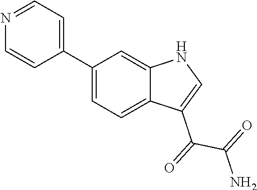 [SMILES: NC(=O)C(=O)c1c[nH]c2cc(-c3ccncc3)ccc12]

To a stirred solution of 6-(pyridin-4-yl)-1H-indole (3.5 g, 18.04 mmol) in dichloromethane (50 ml) was added oxalyl chloride (3.4 g, 27.06 mmol) slowly at 0° C. The resulting reaction mixture was allowed to warm to room temperature, stirred at rt for 12 h and then poured into ammonia water. The solid was filtered, washed with water and dried to give the desired product (3.5 g, yield: 73%) as a white solid.

### Step 3. 2-oxo-2-(1-propyl-6-(pyridin-4-yl)-1H-indol-3-yl)acetamide

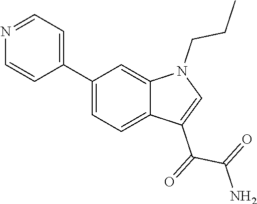 [SMILES: CCCn1cc(C(=O)C(N)=O)c2ccc(-c3ccncc3)cc21]

To a stirred solution of 2-oxo-2-(6-(pyridin-4-yl)-1H-indol-3-yl)acetamide (500 mg, 1.89 mmol) in DMF (10 mL) were added 1-Bromopropane (232 mg, 1.89 mmol) and K₂CO₃ (391 mg, 2.83 mmol). The resulting reaction mixture was stirred at 80° C. for 2 h and cooled down to room temperature at which time water (50 mL) and EA (50 mL) were added. The organic layer was separated and the aqueous layer was extracted with EA (2×30 mL). The combined organic layer was washed with brine (2×50 mL), dried over anhydrous Na₂SO₄ and evaporated to give crude product which was purified by prep-TLC (PE/EA=1:1) to give the desired product (110 mg, yield: 19%) as a yellow solid.

### Step 4. 2-(1-propyl-6-(pyridin-4-yl)-1H-indol-3-yl)ethanamine

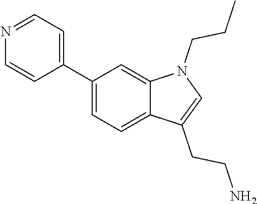 [SMILES: CCCn1cc(CCN)c2ccc(-c3ccncc3)cc21]

A solution of 2-oxo-2-(1-propyl-6-(pyridin-4-yl)-1H-indol-3-yl)acetamide (100 mg, 0.33 mmol) in BH₃ (1 N in THF, 10 mL) was stirred at room temperature for 2 h at which time HCl (0.5 mL, 10 N) was added and the reaction mixture was stirred for another 10 min. The solvent was removed and the residue was purified by prep-HPLC to give the desired compound (80 mg, yield: 88%) as a white solid.

### Step 5. dimethyl({2-[1-propyl-6-(pyridin-4-yl)-1H-indol-3-yl]ethyl}sulfamoyl)amine

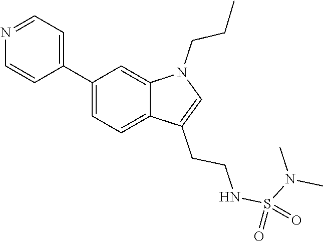 [SMILES: CCCn1cc(CCNS(=O)(=O)N(C)C)c2ccc(-c3ccncc3)cc21]

To a stirred solution of 2-(1-propyl-6-(pyridin-4-yl)-1H-indol-3-yl)ethanamine (80 mg, 0.28 mmol) and TEA (86.86 mg, 0.86 mmol) in DCM (50 mL) was added dimethylsulfamoyl chloride (237 mg, 1.65 mmol) dropwise at room temperature. The mixture was stirred for 1 h and the solvent was removed. The residue was purified by pre-HPLC to give the desired product (9 mg, yield: 8%) as a yellow solid. ¹H NMR (400 MHz, MeOD) δ 8.44 (d, J=5.6 Hz, 2H), 7.67-7.70 (m, 3H), 7.60 (d, J=8.0 Hz, 1H), 7.36 (d, J=8.4 Hz, 1H), 7.13 (s, 1H), 4.09 (t, J=7.2 Hz, 2H), 3.15-3.20 (m, 2H), 2.90 (t, J=7.6 Hz, 2H), 2.59 (s, 6H), 1.70-1.85 (m, 2H), 0.83 (t, J=7.2 Hz, 3H). LCMS (Method A) RT 2.09 min, calcd. for C₂₀H₂₆N₄O₂S 386.18 m/z, found 387.3 m/z [M+H]⁺.

### Example B-7: ({2-[1-(cyclopropylmethyl)-6-(pyridin-4-yl)-1H-indol-3-yl]ethyl}sulfamoyl)dimethylamine

### Step 1. 2-(1-(cyclopropylmethyl)-6-(pyridin-4-yl)-1H-indol-3-yl)-2-oxoacetamide

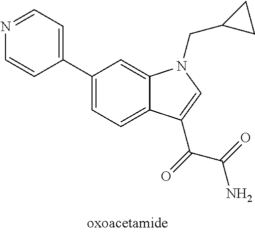 [SMILES: NC(=O)C(=O)c1cn(CC2CC2)c2cc(-c3ccncc3)ccc12]

To a stirred solution of 2-oxo-2-(6-(pyridin-4-yl)-1H-indol-3-yl)acetamide (250 mg, 0.94 mmol) in DMF (5 mL) was added (bromomethyl)cyclopropane (190 mg, 1.42 mmol) and K₂CO₃ (259 mg, 1.88 mmol). The resulting reaction mixture was stirred at 80° C. for 2 h and cooled down to room temperature. Water (30 mL) and EA (30 mL) was then added and the organic layer was separated. The aqueous layer was extracted with EA (2×30 mL), the combined organic layer was washed with brine (2×50 mL), dried over anhydrous Na₂SO₄ and evaporated to give crude product which was purified by prep-TLC (PE/EA=1:1) to give the desired product (110 mg, yield: 37%) as a yellow solid.

### Step 2. 2-(1-(cyclopropylmethyl)-6-(pyridin-4-yl)-1H-indol-3-yl)ethanamine

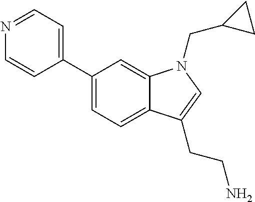 [SMILES: NCCc1cn(CC2CC2)c2cc(-c3ccncc3)ccc12]

A mixture of 2-(1-(cyclopropylmethyl)-6-(pyridin-4-yl)-1H-indol-3-yl)-2-oxoacetamide (110 mg, 0.35 mmol) in BH₃ (1 N in THF, 3 mL) was stirred at room temperature for 2 h at which time, concentrated HCl (12 N, 1 mL) was added. The mixture was then stirred for 10 minutes and then the solvent was removed. The residue was purified by pre-HPLC to give the desired product (70 mg, yield: 69%) as a yellow solid.

### Step 3. ({2-[1-(cyclopropylmethyl)-6-(pyridin-4-yl)-1H-indol-3-yl]ethyl}sulfamoyl)dimethylamine

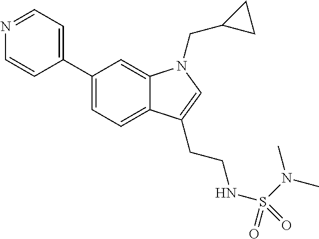 [SMILES: CN(C)S(=O)(=O)NCCc1cn(CC2CC2)c2cc(-c3ccncc3)ccc12]

To a mixture of 2-(1-(cyclopropylmethyl)-6-(pyridin-4-yl)-1H-indol-3-yl)ethanamine (70 mg, 0.24 mmol) and TEA (72.89 mg, 0.72 mmol) in DCM (5 mL) was added dimethylsulfamoyl chloride (68.64 mg, 0.48 mmol) dropwise at room temperature. The mixture was stirred for 2 h at which time the solvent was removed. The residue was purified by prep HPLC to give ({2-[1-(cyclopropylmethyl)-6-(pyridin-4-yl)-1H-indol-3-yl]ethyl}sulfamoyl)dimethylamine (64.47 mg, yield: 67%) as a yellow solid. ¹H NMR (400 MHz, MeOD) δ 8.44 (d, J=5.2 Hz, 2H), 7.67-7.73 (m, 3H), 7.60 (d, J=8.0 Hz, 1H), 7.37 (d, J=8.4 Hz, 1H), 7.21 (s, 1H), 3.99 (d, J=7.2 Hz, 2H), 3.18-3.20 (m, 2H), 2.90 (t, J=7.6 Hz, 2H), 2.59 (s, 6H), 1.18-1.21 (m, 1H), 0.48-0.52 (m, 2H), 0.30-0.35 (m, 2H). LC-MS (Method B), RT 1.94 min; MS Calcd. For C₂₁H₂₆N₄O₂S: 398.2; MS Found: 398.4 [M+H]⁺.

### Example B-8: ({2-[1-benzyl-6-(pyridin-4-yl)-1H-indol-3-yl]ethyl}sulfamoyl)dimethylamine

### Step 1. 2-(1-benzyl-6-(pyridin-4-yl)-1H-indol-3-yl)-2-oxoacetamide

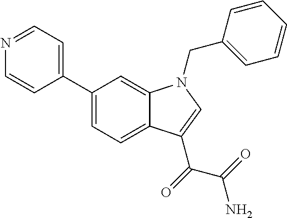 [SMILES: NC(=O)C(=O)c1cn(Cc2ccccc2)c2cc(-c3ccncc3)ccc12]

To a stirred solution of 2-oxo-2-(6-(pyridin-4-yl)-1H-indol-3-yl)acetamide (250 mg, 0.94 mmol) in DMF (5 mL) were added (bromomethyl)benzene (241.4 mg, 1.42 mmol) and K₂CO₃ (259 mg, 1.88 mmol). The resulting reaction mixture was stirred at 80° C. for 2 h and cooled down to room temperature at which time water (30 mL) and EA (30 mL) were added. The organic layer was separated and the aqueous layer was extracted with EA (2×30 mL). The combined organic layer was washed with brine (2×50 mL), dried over anhydrous Na₂SO₄ and evaporated to give crude product which was purified by prep-TLC (PE/EA=1:1) to give the desired product (150 mg, yield: 44%) as a yellow solid.

### Step 2. 2-(1-benzyl-6-(pyridin-4-yl)-1H-indol-3-yl)ethanamine

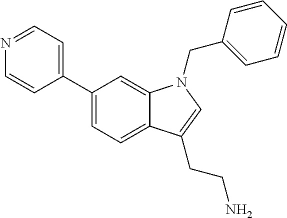 [SMILES: NCCc1cn(Cc2ccccc2)c2cc(-c3ccncc3)ccc12]

A mixture of 2-(1-benzyl-6-(pyridin-4-yl)-1H-indol-3-yl)-2-oxoacetamide (150 mg, 0.42 mmol) in BH₃ (1 N in THF, 3 mL) was stirred at room temperature for 2 h. at which time concentrated HCl (12 N, 1 mL) was added. The mixture was stirred for 10 min and then the solvent was removed. The residue was purified by pre-HPLC to give the desired product (110 mg, yield: 80%) as a yellow solid.

### Step 3. ({2-[1-benzyl-6-(pyridin-4-yl)-1H-indol-3-yl]ethyl}sulfamoyl)dimethylamine

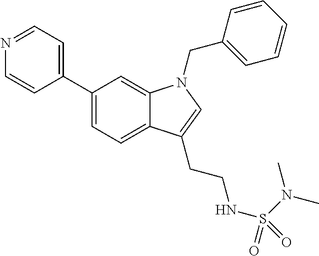 [SMILES: CN(C)S(=O)(=O)NCCc1cn(Cc2ccccc2)c2cc(-c3ccncc3)ccc12]

To a mixture of 2-(1-benzyl-6-(pyridin-4-yl)-1H-indol-3-yl)ethanamine (110 mg, 0.34 mmol) and TEA (67.95 mg, 0.67 mmol) in DCM (5 mL) was added dimethylsulfamoyl chloride (68.64 mg, 0.51 mmol) dropwise at room temperature. The mixture was stirred for 2 h at which time the solvent was removed. The residue was purified by pre-HPLC to give the desired product (7.84 mg, yield: 5%) as a yellow solid. ¹H NMR (400 MHz, MeOD) δ 8.53 (d, J=5.6 Hz, 2H), 7.71-7.76 (m, 4H), 7.50 (d, J=9.2 Hz, 1H), 7.20-7.32 (m, 6H), 5.46 (s, 2H), 3.32-3.33 (m, 2H), 3.04 (d, J=7.2 Hz, 2H), 2.69 (s, 6H). LCMS (Method B) RT 2.04 min, calcd. for C₂₄H₂₆N₄O₂S 434.18 m/z, found 435.3 m/z [M+H]⁺.

### Example B-9: dimethyl[(2-{1-[(oxolan-3-yl)methyl]-6-(pyridin-4-yl)-1H-indol-3-yl}ethyl)sulfamoyl]amine

### Step 1. 2-oxo-2-(6-(pyridin-4-yl)-1-((tetrahydrofuran-3-yl)methyl)-1H-indol-3-yl)acetamide

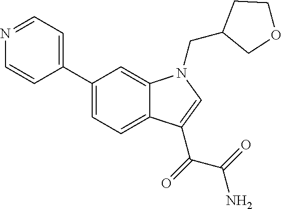 [SMILES: NC(=O)C(=O)c1cn(CC2CCOC2)c2cc(-c3ccncc3)ccc12]

To a stirred solution of 2-oxo-2-(6-(pyridin-4-yl)-1H-indol-3-yl)acetamide (250 mg, 0.94 mmol) in DMF (5 mL) were added 3-(bromomethyl)tetrahydrofuran (233 mg, 1.42 mmol) and K₂CO₃ (259 mg, 1.88 mmol). The resulting reaction mixture was stirred at 80° C. for 2 h and cooled down to room temperature. Water (30 mL) and EA (30 mL) was added. The organic layer was separated and the aqueous layer was extracted with EA (2×30 mL). The combined organic layer was washed with brine (2×50 mL), dried over anhydrous Na₂SO₄ and evaporated to give crude product which was purified by prep-TLC (PE/EA=1:1) to give the desired product (110 mg, yield: 33.4%) as a yellow solid.

### Step 2. 2-(6-(pyridin-4-yl)-1-((tetrahydrofuran-3-yl)methyl)-1H-indol-3-yl)ethanamine

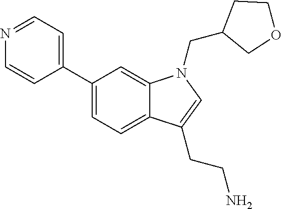 [SMILES: NCCc1cn(CC2CCOC2)c2cc(-c3ccncc3)ccc12]

To a suspension of 2-oxo-2-(6-(pyridin-4-yl)-1-((tetrahydrofuran-3-yl)methyl)-1H-indol-3-yl)acetamide (110 mg, 0.32 mmol) in dioxane (3 mL), was added BH₃ (1 N in THF, 3 mL) at room temperature. The mixture was stirred at 65° C. for 2 h and cooled down to room temperature. Con. HCl (12 N, 1 mL) was then added. The mixture was stirred for another 10 min, and then the solvent was removed. The residue was purified by pre-HPLC to give the desired product (95 mg, yield: 94%) as a yellow solid.

### Step 3. 2-oxo-2-(6-(pyridin-4-yl)-1-((tetrahydrofuran-3-yl)methyl)-1H-indol-3-yl)acetamide

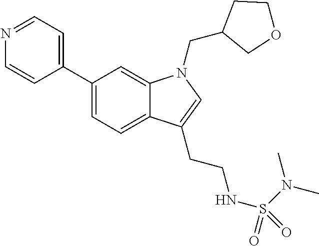 [SMILES: CN(C)S(=O)(=O)NCCc1cn(CC2CCOC2)c2cc(-c3ccncc3)ccc12]

To a mixture of 2-(6-(pyridin-4-yl)-1-((tetrahydrofuran-3-yl)methyl)-1H-indol-3-yl)ethanamine (95 mg, 0.30 mmol) and TEA (89.67 mg, 0.89 mmol) in DMF (5 mL), was added dimethylsulfamoyl chloride (64.35 mg, 0.45 mmol) dropwise at room temperature. The mixture was stirred at 70° C. for 2 h, cooled down to room temperature and purified by pre-HPLC to give the desired product (110 mg, yield: 87%) as a yellow solid. ¹H NMR (400 MHz, MeOD) δ 8.52-8.63 (m, 2H), 7.81-7.86 (m, 3H), 7.73 (d, J=8.4 Hz, 1H), 7.50 (d, J=8.0 Hz, 1H), 7.28 (s, 1H), 4.24 (d, J=8.0 Hz, 2H), 3.95-4.23 (m, 1H), 3.72-3.81 (m, 2H), 3.58-3.63 (m, 1H), 3.31-3.32 (m, 2H), 3.02 (t, J=7.2 Hz, 2H), 2.85-2.95 (m, 1H), 2.72 (s, 6H), 2.01-2.11 (m, 1H), 1.72-1.81 (m, 1H). LCMS (Method A) RT=1.90 calcd. for C₂₂H₂₈N₄O₃S 428.19 m/z, found 429.2 m/z [M+H]⁺.

### Example B-11: N-{2-[1-(2-methylpropyl)-6-(pyridin-4-yl)-1H-indol-3-yl]ethyl}cyclopentanesulfonamide

### Step 1. 6-bromo-1-isobutyl-1H-indole

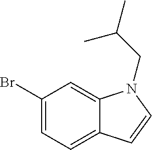

To a stirred solution of 6-bromo-1H-indole (5.0 g, 25.5 mmol) in DMF (100 mL) was added Cs₂CO₃ (24.9 g, 76.5 mmol) and 1-bromo-2-methylpropane (10.5 g, 76.5 mmol) at rt. The resulting reaction mixture was stirred for 6 h at 70° C. Then water was added, the aqueous phase was extracted with dichloromethane, the organic phases was dried over anhydrous sodium sulfate, filtered and concentrated in vacuo. The crude product was purified by prep-HPLC to give the desired product (5.7 g, yield: 89%) as a yellow solid.

### Step 2. 1-isobutyl-6-(pyridin-4-yl)-1H-indole

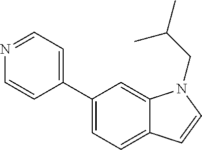 [SMILES: CC(C)Cn1ccc2ccc(-c3ccncc3)cc21]

To a stirred solution of 6-bromo-1-isobutyl-1H-indole (5.7 g, 22.7 mmol) in DMF (100 mL) was added Pd(PPh₃)₄ (5.2 g, 4.5 mmol), Cs₂CO₃ (11.1 g, 34.1 mmol) and 4-(4,4,5,5-tetramethyl-1,3,2-dioxaborolan-2-yl)pyridine (4.7 g, 22.7 mmol). The resulting reaction mixture was heated to 90° C., stirred for 4 h and concentrated in vacuo to remove the solvent. Water was then added, the aqueous phase was extracted with dichloromethane, the organic phases was dried over anhydrous sodium sulfate, filtered and concentrated. The crude was purified by prep-HPLC to give the desired product (5.1 g, yield: 89%) as a yellow solid.

### Step 3. 2-(1-isobutyl-6-(pyridin-4-yl)-1H-indol-3-yl)-2-oxoacetamide

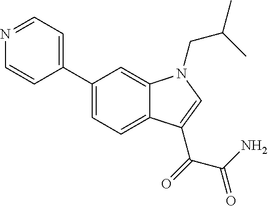 [SMILES: CC(C)Cn1cc(C(=O)C(N)=O)c2ccc(-c3ccncc3)cc21]

To a stirred solution of 1-isobutyl-6-phenyl-1H-indole (5.1 g, 20.4 mmol) in DCM (100 ml) was added Oxalyl chloride (7.8 g, 61.2 mmol) at 0° C. The resulting reaction mixture was stirred at rt for 0.5 h. Then an ammonia solution was added, the aqueous phase was extracted with dichloromethane, the organic phases was dried over anhydrous sodium sulfate, filtered and concentrated in vacuo to give the desired product (4.0 g, yield: 61%) as a white solid.

### Step 4. 2-(1-isobutyl-6-(pyridin-4-yl)-1H-indol-3-yl)ethanamine

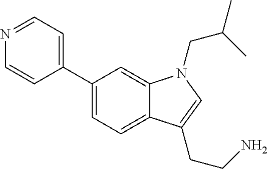 [SMILES: CC(C)Cn1cc(CCN)c2ccc(-c3ccncc3)cc21]

To a stirred solution of 2-(1-isobutyl-6-(pyridin-4-yl)-1H-indol-3-yl)-2-oxoacetamide (4.0 g, 12.5 mmol) in THF (100 ml) was added B₂H₆/THF (1 M, 100 mL) at rt. The resulting reaction mixture was heated to 70° C. for 16 h. HCl (1.0 N, 3 mL) was then added and stirred for 1 h at rt at which time the aqueous phase was neutralized and extracted with dichloromethane. The organic phases was dried over anhydrous sodium sulfate, filtered and concentrated in vacuo. The crude was purified by prep-HPLC to give the desired product (1.9 g, yield: 51%) as a yellow solid.

### Step 5. N-(2-(1-isobutyl-6-(pyridin-4-yl)-1H-indol-3-yl)ethyl)cyclopentanesulfonamide

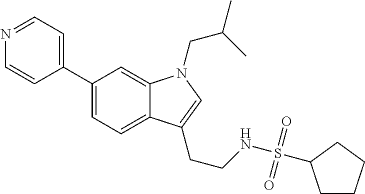 [SMILES: CC(C)Cn1cc(CCNS(=O)(=O)C2CCCC2)c2ccc(-c3ccncc3)cc21]

To a stirred solution of 2-(1-isobutyl-6-(pyridin-4-yl)-1H-indol-3-yl)ethanamine (100 mg, 0.34 mmol) in MeCN (5 mL) were added cyclopentanesulfonyl chloride (86 mg, 0.51 mmol) and DIEA (132 mg, 1.02 mmol). The resulting reaction mixture was stirred for 12 h at rt. Water was then added, the aqueous phase was extracted with DCM, the organic phases was dried over anhydrous sodium sulfate, filtered and concentrated in vacuo. The crude was purified by prep-TLC to give the desired product (17 mg, yield: 11.7%) as a yellow solid. ¹H NMR (400 MHz, DMSO-d₆) δ 8.60 (d, J=4.4 Hz, 2H), 7.93 (d, J=1.2 Hz, 1H), 7.78 (d, J=4.8 Hz, 2H), 7.64 (d, J=8.4 Hz, 1H), 7.47 (d, J=8.4 Hz, 1H), 7.33 (s, 1H), 7.15 (s, 1H), 4.03 (d, J=7.2 Hz, 2H), 3.49-3.51 (m, 1H), 3.10-3.18 (m, 2H), 2.90 (d, J=7.6 Hz, 2H), 2.09-2.20 (m, 1H), 1.75-1.93 (m, 4H), 1.45-1.70 (m, 4H), 0.87 (d, J=6.8 Hz, 6H). LC-MS (Method B), Purity: 100.0%, Rt=2.13 min; MS Calcd. for C₂₄H₃₁N₃O₂S: 425.2; MS Found: 426.2 [M+H]⁺.

### Example B-12: 2-methyl-N-{2-[1-(2-methylpropyl)-6-(pyridin-4-yl)-1H-indol-3-yl]ethyl}propane-1-sulfonamide

### Step 1 N-(2-(1-isobutyl-6-(pyridin-4-yl)-1H-indol-3-yl)ethyl)-2-methylpropane-1-sulfonamide

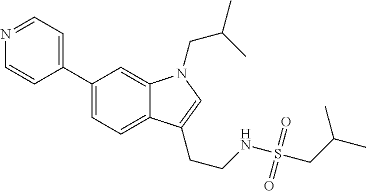 [SMILES: CC(C)Cn1cc(CCNS(=O)(=O)CC(C)C)c2ccc(-c3ccncc3)cc21]

To a stirred solution of 2-(1-isobutyl-6-(pyridin-4-yl)-1H-indol-3-yl)ethan-1-amine (100 mg, 0.34 mmol) in MeCN (5 mL) was added 2-methylpropane-1-sulfonyl chloride (80 mg, 0.51 mmol) and DIEA (132 mg, 1.02 mmol). The resulting reaction mixture was stirred for 12 h at rt. Water was then added, the aqueous phase was extracted with DCM, the organic phases was dried over anhydrous sodium sulfate, filtered, concentrated in vacuo and purified by prep-TLC to give the desired product (48 mg, yield: 34%) as a white solid. ¹H NMR (400 MHz, DMSO-d₆) δ 8.60 (d, J=4.8 Hz, 2H), 7.93 (d, J=0.8 Hz, 1H), 7.78 (d, J=4.8 Hz, 2H), 7.65 (d, J=8.4 Hz, 1H), 7.47 (d, J=8.4 Hz, 1H), 7.33 (s, 1H), 7.15 (t, J=6.0 Hz, 1H), 4.03 (d, J=7.2 Hz, 2H), 3.18-3.35 (m, 2H), 2.86-2.94 (m, 2H), 2.81 (d, J=6.4 Hz, 2H), 1.95-2.20 (m, 2H), 0.96 (d, J=6.8 Hz, 6H), 0.87 (d, J=6.4 Hz, 6H). LC-MS (Method B), Purity: 100.00%, Rt=2.14 min; MS Calcd. for C₂₃H₃₁N₃O₂S: 413.2; MS Found: 414.3 [M+H]⁺.

### Example B-13: N-{2-[1-(2-methylpropyl)-6-(pyridin-4-yl)-1H-indol-3-yl]ethyl}cyclopropanesulfonamide

### Step 1. N-(2-(1-isobutyl-6-(pyridin-4-yl)-1H-indol-3-yl)ethyl)cyclopropanesulfonamide

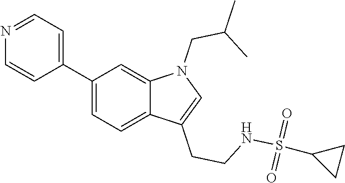 [SMILES: CC(C)Cn1cc(CCNS(=O)(=O)C2CC2)c2ccc(-c3ccncc3)cc21]

To a stirred solution of 2-(1-isobutyl-6-(pyridin-4-yl)-1H-indol-3-yl)ethan-1-amine (100 mg, 0.34 mmol) in MeCN (5 mL) was added cyclopropanesulfonyl chloride (72 mg, 0.51 mmol) and DIEA (132 mg, 1.02 mmol). The resulting reaction mixture was stirred for 12 h at rt. Water was then added, the aqueous phase was extracted with DCM, the organic phases was dried over anhydrous sodium sulfate, filtered, concentrated in vacuo and purified by prep-TLC to give the desired product (41 mg, yield: 30%) as a pink solid. ¹H NMR (400 MHz, DMSO-d₆) δ 8.60 (d, J=4.4 Hz, 2H), 7.93 (d, J=1.2 Hz, 1H), 7.78 (d, J=4.8 Hz, 2H), 7.64 (d, J=8.0 Hz, 1H), 7.47 (d, J=8.0 Hz, 1H), 7.33 (s, 1H), 7.15 (t, J=6.0 Hz, 1H), 4.03 (d, J=7.2 Hz, 2H), 3.20-3.30 (m, 2H), 2.92 (t, J=8.0 Hz, 2H), 2.52-2.56 (m, 1H), 2.10-2.20 (m, 1H), 0.82-0.94 (m, 10H). LC-MS (Method B), Purity: 100.00%, Rt=1.974 min; MS Calcd. for C₂₂H₂₇N₃O₂S: 397.2; MS Found: 398.2 [M+H]⁺.

### Example B-14: 3,5-dimethyl-N-{2-[1-(2-methylpropyl)-6-(pyridin-4-yl)-1H-indol-3-yl]ethyl}-1,2-oxazole-4-sulfonamide

### Step 1. N-(2-(1-isobutyl-6-(pyridin-4-yl)-1H-indol-3-yl)ethyl)-3,5-dimethylisoxazole-4-sulfonamide

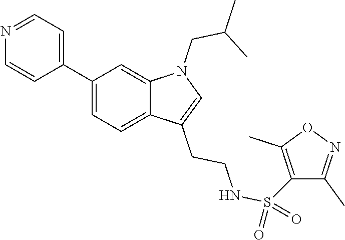 [SMILES: Cc1noc(C)c1S(=O)(=O)NCCc1cn(CC(C)C)c2cc(-c3ccncc3)ccc12]

To a stirred solution of 2-(1-isobutyl-6-(pyridin-4-yl)-1H-indol-3-yl)ethan-1-amine (100 mg, 0.34 mmol) in MeCN (5 mL) was added 3,5-dimethylisoxazole-4-sulfonyl chloride (100 mg, 0.51 mmol) and DIEA (132 mg, 1.02 mmol). The resulting reaction mixture was stirred for 12 h at rt. Water was then added, the aqueous phase was extracted with DCM, the organic phases were dried over anhydrous sodium sulfate, filtered, concentrated in vacuo and purified by prep-TLC to give the desired product (33 mg, yield: 21%) as a white solid. ¹H NMR (400 MHz, DMSO-d₆) δ 8.60 (d, J=4.8 Hz, 2H), 8.02 (d, J=6.0 Hz, 1H), 7.93 (d, J=0.8 Hz, 1H), 7.78 (d, J=4.8 Hz, 2H), 7.58 (d, J=8.4 Hz, 1H), 7.45 (d, J=8.4 Hz, 1H), 7.34 (s, 1H), 4.00 (d, J=7.6 Hz, 2H), 3.08-3.20 (m, 2H), 2.86 (t, J=7.2 Hz, 2H), 2.50-2.52 (m, 3H), 2.27 (s, 3H), 2.08-2.20 (m, 1H), 0.86 (d, J=6.8 Hz, 6H). LC-MS (Method B), Purity: 99.17%, Rt=2.10 min; MS Calcd for C₂₄H₂₈N₄O₃S.: 452.2; MS Found: 453.1 [M+H]⁺.

### Example B-15: N-{2-[1-(2-methylpropyl)-6-(pyridin-4-yl)-1H-indol-3-yl]ethyl}pyridine-3-sulfonamide

### Step 1. N-(2-(1-isobutyl-6-(pyridin-4-yl)-1H-indol-3-yl)ethyl)pyridine-3-sulfonamide

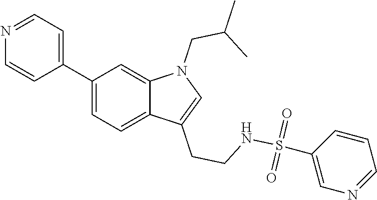 [SMILES: CC(C)Cn1cc(CCNS(=O)(=O)c2cccnc2)c2ccc(-c3ccncc3)cc21]

To a stirred solution of 2-(1-isobutyl-6-(pyridin-4-yl)-1H-indol-3-yl)ethan-1-amine (100 mg, 0.34 mmol) in MeCN (5 mL) was added pyridine-3-sulfonyl chloride hydrochloride (150 mg, 0.70 mmol) and DIEA (132 mg, 1.02 mmol). The resulting reaction mixture was stirred for 12 h at rt. Water was then add, the aqueous phase was extracted with DCM, the organic phases were dried over anhydrous sodium sulfate, filtered, concentrated in vacuo and purified by prep-TLC to give the desired product (50 mg, yield: 34%) as a white solid. ¹H NMR (400 MHz, DMSO-d₆) δ 8.92-8.93 (m, 1H), 8.78 (d, J=4.8 Hz, 1H), 8.60 (d, J=4.4 Hz, 2H), 8.11-8.14 (m, 1H), 8.02 (s, 1H), 7.90 (d, J=0.8 Hz, 1H), 7.78 (d, J=4.4 Hz, 2H), 7.52-7.60 (m, 2H), 7.44 (d, J=8.4 Hz, 1H), 7.24 (s, 1H), 4.00 (d, J=7.6 Hz, 2H), 3.10 (t, J=7.2 Hz, 2H), 2.83 (t, J=7.2 Hz, 2H), 2.07-2.14 (m, 1H), 0.86 (d, J=6.4 Hz, 6H). LC-MS (Method B), Purity: 100.00%, Rt=1.929 min; MS Calcd.: 434.2; MS Found: 435.3 [M+H]⁺.

### Example B-16: ({2-[6-cyclohexyl-1-(2-methylpropyl)-1H-indol-3-yl]ethyl}sulfamoyl)dimethylamine

### Step 1. ({2-[6-(cyclohex-1-en-1-yl)-1-(2-methylpropyl)-1H-indol-3-yl]ethyl}sulfamoyl)dimethylamine

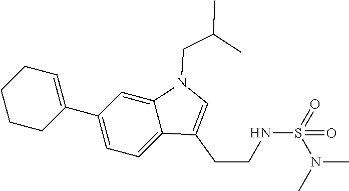 [SMILES: CC(C)Cn1cc(CCNS(=O)(=O)N(C)C)c2ccc(C3=CCCCC3)cc21]

To a solution of 2-(cyclohex-1-en-1-yl)-4,4,5,5-tetramethyl-1,3,2-dioxaborolane (74.69 mg, 0.36 mmol), ({2-[6-bromo-1-(2-methylpropyl)-1H-indol-3-yl]ethyl}sulfamoyl)dimethylamine (120 mg, 0.30 mmol) and K₃PO₄·7H₂O (304.20 mg, 0.90 mmol) in dioxane (5.00 mL), was added Pd(dppf)Cl₂ (30.00 mg) under N₂ atmosphere. The reaction mixture was stirred at 90° C. for 2 h, at which time the solvent was removed. The residue was purified by pre-HPLC to give the desired product (75.00 mg, yield: 62%) as a white solid.

### Step 2. ({2-[6-cyclohexyl-1-(2-methylpropyl)-1H-indol-3-yl]ethyl}sulfamoyl)dimethylamine

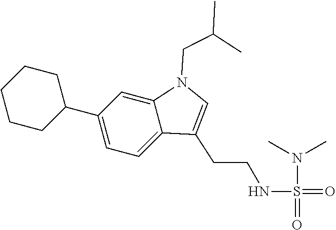 [SMILES: CC(C)Cn1cc(CCNS(=O)(=O)N(C)C)c2ccc(C3CCCCC3)cc21]

To a mixture of ({2-[6-(cyclohex-1-en-1-yl)-1-(2-methylpropyl)-1H-indol-3-yl]ethyl}sulfamoyl)dimethylamine (75.0 mg, 0.186 mmol) in MeOH (5 mL) was added Pd/C (10 mg) at which time the mixture was reacted under H₂ (1.0 atm) for 1 h. The solid was filtered off and the filtrate was evaporated to give the desired product (64.8 mg, yield: 86%) as a white solid. ¹H NMR (400 MHz, d₆-DMSO): δ 7.38 (d, J=8.0 Hz, 1H), 7.29 (t, J=6.0 Hz, 1H), 7.22 (s, 1H), 7.10 (s, 1H), 6.89 (dd, J=8.4 Hz, 1.2 Hz 1H), 3.87 (d, J=7.2 Hz, 2H), 3.10-3.16 (m, 2H), 2.83 (t, J=8.4 Hz, 2H), 2.50-2.59 (m, 7H), 2.04-2.11 (m, 1H), 1.70-1.82 (m, 5H), 1.33-1.52 (m, 5H), 0.87 (t, J=7.2 Hz, 6H). LCMS (Method B), RT 2.45 min, calcd. for C₂₂H₃₅N₃O₂S 405.24 m/z, found 406.4 m/z [M+H]⁺.

### Example B-17: dimethyl({2-[1-(2-methylpropyl)-6-[2-(trifluoromethyl)pyridin-4-yl]-1H-indol-3-yl]ethyl}sulfamoyl)amine

### Step 1. dimethyl({2-[1-(2-methylpropyl)-6-[2-(trifluoromethyl)pyridin-4-yl]-1H-indol-3-yl]ethyl}sulfamoyl)amine

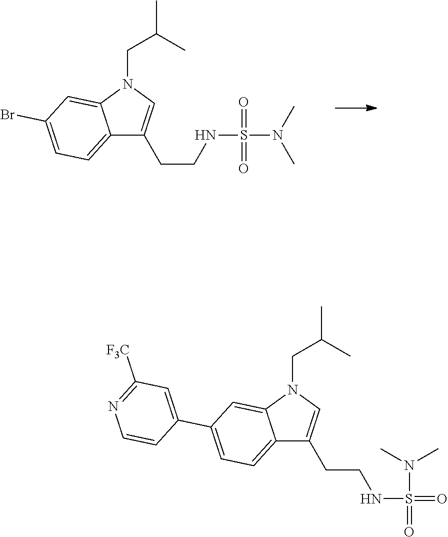 [SMILES: CC(C)Cn1cc(CCNS(=O)(=O)N(C)C)c2ccc(Br)cc21 ; CC(C)Cn1cc(CCNS(=O)(=O)N(C)C)c2ccc(-c3ccnc(C(F)(F)F)c3)cc21]

To a solution of 4-(4,4,5,5-tetramethyl-1,3,2-dioxaborolan-2-yl)-2-(trifluoromethyl)pyridine (81.70 mg, 0.30 mmol), ({2-[6-bromo-1-(2-methylpropyl)-1H-indol-3-yl]ethyl}sulfamoyl)dimethylamine (100 mg, 0.25 mmol) and K₃PO₄·7H₂O (253.50 mg, 0.75 mmol) in dioxane (5.00 mL), was added Pd(dppf)Cl₂ (10.00 mg) under N₂ atmosphere. The reaction mixture was stirred at 90° C. for 2 h at which time the solvent was removed. The residue was purified by pre-HPLC to give the desired product (65.40 mg, yield: 56%) as a white solid. ¹H NMR (400 MHz, d₆-DMSO): δ 8.78 (d, J=5.2 Hz, 1H), 8.24 (d, J=1.2 Hz, 1H), 8.13 (dd, J=4.8 Hz, 1.2 Hz, 1H), 8.09 (d, J=1.2 Hz, 1H), 7.67 (d, J=8.4 Hz, 1H), 7.57 (dd, J=8.4 Hz, 1.2 Hz 1H), 7.37 (s, 1H), 7.32 (t, J=6.0 Hz, 1H), 4.06 (d, J=7.2 Hz, 2H), 3.16-3.21 (m, 2H), 2.90 (t, J=7.6 Hz, 2H), 2.63 (s, 6H), 2.14-2.18 (m, 1H), 0.87 (d, J=6.8 Hz, 6H). LCMS (Method B), RT 2.34 min, calcd. for C₂₂H₂₇F₃N₄O₂S 468.18 m/z, found 469.1 m/z [M+H]⁺.

### Example B-18: 4-(3-{2-[(dimethylsulfamoyl)amino]ethyl}-1-(2-methylpropyl)-1H-indol-6-yl)-1,2-dihydropyridin-2-one

### Step 1. dimethyl({2-[1-(2-methylpropyl)-6-(4,4,5,5-tetramethyl-1,3,2-dioxaborolan-2-yl)-1H-indol-3-yl]ethyl}sulfamoyl)amine

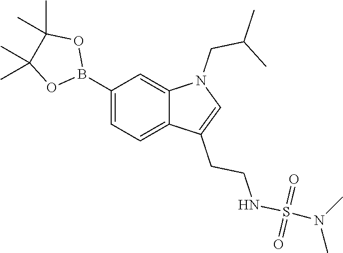

To a solution of 4,4,4′,4′,5,5,5′,5′-octamethyl-2,2′-bi(1,3,2-dioxaborolane) (114.02 mg, 0.45 mmol), ({2-[6-bromo-1-(2-methylpropyl)-1H-indol-3-yl]ethyl}sulfamoyl)dimethylamine (150 mg, 0.37 mmol) and KOAc (108.78 mg, 1.11 mmol) in toluene (5.00 mL), was added Pd(dppf)Cl₂ (10.00 mg) under N₂ atmosphere. The reaction mixture was stirred at 110° C. for 2 h, at which time the solvent was removed. The residue was purified by pre-TLC (PE/EA=2:1) to give the desired product (110.00 mg, yield: 65%) as a white solid.

### Step 2: 4-(3-{2-[(dimethylsulfamoyl)amino]ethyl}-1-(2-methylpropyl)-1H-indol-6-yl)-1,2-dihydropyridin-2-one

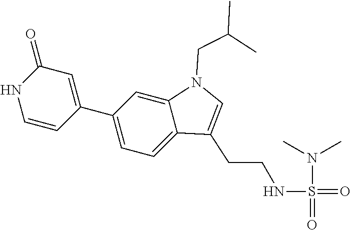 [SMILES: CC(C)Cn1cc(CCNS(=O)(=O)N(C)C)c2ccc(-c3cc[nH]c(=O)c3)cc21]

To a solution of 4-bromopyridin-2(1H)-one (50.86 mg, 0.29 mmol), dimethyl({2-[1-(2-methylpropyl)-6-(4,4,5,5-tetramethyl-1,3,2-dioxaborolan-2-yl)-1H-indol-3-yl]ethyl}sulfamoyl)amine (110 mg, 0.24 mmol) and K₃PO₄·7H₂O (108.78 mg, 1.11 mmol) in dioxane (5.00 mL), was added Pd(dppf)Cl₂ (15.00 mg) under N₂ atmosphere. The reaction mixture was stirred at 90° C. for 2 h at which time the solvent was removed. The residue was purified by pre-HPLC to give the desired product (42.11 mg, yield: 41%) as a white solid. ¹H NMR (400 MHz, d₆-DMSO): δ 11.49 (s, 1H), 7.83 (s, 1H), 7.58 (d, J=8.4 Hz, 1H), 7.42 (d, J=6.8 Hz, 1H), 7.29-7.36 (m, 3H), 6.67 (d, J=1.2 Hz, 1H), 6.62 (dd, J=6.8 Hz, 1.6 Hz 1H), 4.02 (d, J=7.2 Hz, 2H), 3.14-3.19 (m, 2H), 2.88 (t, J=8.0 Hz, 2H), 2.63 (s, 6H), 2.07-2.14 (m, 1H), 0.85 (d, J=6.4 Hz, 6H). LCMS (Method B), RT 1.79 min, calcd. for C₂₁H₂₈N₄O₃S 416.19 m/z, found 417.0 m/z [M+H]⁺.

### Example B-19: ({2-[6-(2-methoxypyridin-4-yl)-1-(2-methylpropyl)-1H-indol-3-yl]ethyl}sulfamoyl)dimethylamine

### Step 1. ({2-[6-(2-methoxypyridin-4-yl)-1-(2-methylpropyl)-1H-indol-3-yl]ethyl}sulfamoyl)dimethylamine

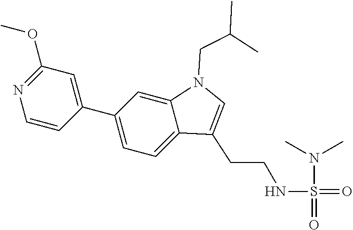 [SMILES: COc1cc(-c2ccc3c(CCNS(=O)(=O)N(C)C)cn(CC(C)C)c3c2)ccn1]

To a solution of 2-methoxy-4-(4,4,5,5-tetramethyl-1,3,2-dioxaborolan-2-yl)pyridine (84.39 mg, 0.36 mmol), ({2-[6-bromo-1-(2-methylpropyl)-1H-indol-3-yl]ethyl}sulfamoyl)dimethylamine (120 mg, 0.30 mmol) and K₃PO₄·7H₂O (304.20 mg, 0.90 mmol) in dioxane (5.00 mL), was added Pd(dppf)Cl₂ (15.00 mg) under N₂ atmosphere. The reaction mixture was stirred at 90° C. for 2 h at which time the solvent was removed. The residue was purified by pre-HPLC to give the desired product (51.04 mg, yield: 40%) as a white solid. ¹H NMR (400 MHz, d₆-DMSO): δ 8.20 (d, J=5.2 Hz, 1H), 7.91 (d, J=0.8 Hz, 1H), 7.60 (d, J=8.4 Hz, 1H), 7.39-7.45 (m, 2H), 7.31 (s, 2H), 7.19 (d, J=0.8 Hz, 1H), 4.03 (d, J=7.6 Hz, 2H), 3.09 (s, 3H), 3.15-3.19 (m, 2H), 2.89 (t, J=8.0 Hz, 2H), 2.63 (s, 6H), 2.12-2.15 (m, 1H), 0.86 (d, J=6.4 Hz, 6H). LCMS (Method B), RT 2.204 min, calcd. for C₂₂H₃₀N₄O₃S 430.20 m/z, found 431.4 m/z [M+H]⁺.

### Example B-20: ({2-[6-(2-chloropyridin-4-yl)-1-(2-methylpropyl)-1H-indol-3-yl]ethyl}sulfamoyl)dimethylamine

### Step 1. The synthesis of ({2-[6-(2-chloropyridin-4-yl)-1-(2-methylpropyl)-1H-indol-3-yl]ethyl}sulfamoyl)dimethylamine

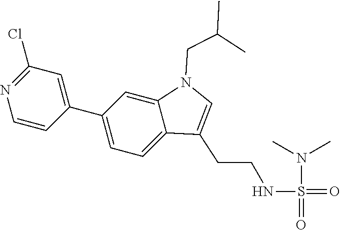 [SMILES: CC(C)Cn1cc(CCNS(=O)(=O)N(C)C)c2ccc(-c3ccnc(Cl)c3)cc21]

To a solution of 2-chloro-4-(4,4,5,5-tetramethyl-1,3,2-dioxaborolan-2-yl)pyridine (86.04 mg, 0.36 mmol), ({2-[6-bromo-1-(2-methylpropyl)-1H-indol-3-yl]ethyl}sulfamoyl)dimethylamine (120 mg, 0.30 mmol) and K₃PO₄·7H₂O (304.20 mg, 0.90 mmol) in dioxane (5.00 mL), was added Pd(dppf)Cl₂ (15.00 mg) under N₂ atmosphere. The reaction mixture was stirred at 90° C. for 2 h at which time the solvent was removed. The residue was purified by pre-HPLC to give the desired product (38.77 mg, yield: 30%) as a yellow solid. ¹H NMR (400 MHz, d₆-DMSO): δ 8.42 (d, J=5.2 Hz, 1H), 8.03 (d, J=1.2 Hz, 1H), 7.94 (d, J=1.2 Hz, 1H), 7.84 (dd, J=5.6 Hz, 1.6 Hz, 1H), 7.64 (d, J=8.4 Hz, 1H), 7.51 (dd, J=8.4 Hz, 1.6 Hz, 1H), 7.36 (s, 1H), 7.32 (s, 1H), 4.05 (d, J=7.6 Hz, 2H), 3.16-3.19 (m, 2H), 2.89 (t, J=8.0 Hz, 2H), 2.63 (s, 6H), 2.13-2.17 (m, 1H), 0.86 (d, J=6.8 Hz, 6H). LCMS (Method B), RT 2.20 min, calcd. for C₂₁H₂₇ClN₄O₂S 434.15 m/z, found 435.3 m/z [M+H]⁺.

### Example B-21: 4-(3-{2-[(dimethylsulfamoyl)amino]ethyl}-1-(2-methylpropyl)-1H-indol-6-yl)pyridin-2-amine

### Step 1. 2-aminopyridin-4-ylboronic acid

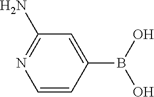

To a solution of 4,4,4′,4′,5,5,5′,5′-octamethyl-2,2′-bi(1,3,2-dioxaborolane) (158.75 mg, 0.63 mmol), 4-bromopyridin-2-amine (100 mg, 0.52 mmol) and KOAc (152.88 mg, 1.56 mmol) in DMSO (2.00 mL), was added Pd(dppf)Cl₂ (10.00 mg) under N₂ atmosphere. The reaction mixture was stirred at 120° C. under MW for 1 h and cooled down to room temperature. The crude mixture was used to the next step directly.

### Step 2. 4-(3-{2-[(dimethylsulfamoyl)amino]ethyl}-1-(2-methylpropyl)-1H-indol-6-yl)pyridin-2-amine

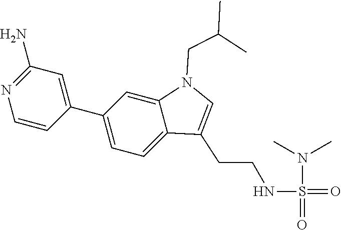 [SMILES: CC(C)Cn1cc(CCNS(=O)(=O)N(C)C)c2ccc(-c3ccnc(N)c3)cc21]

To a mixture of ({2-[6-bromo-1-(2-methylpropyl)-1H-indol-3-yl]ethyl}sulfamoyl)dimethylamine (104.26 mg, 0.26 mmol), 2-aminopyridin-4-ylboronic acid (71.76 mg, 0.52 mmol) and K₃PO₄·7H₂O (263.64 mg, 0.78 mmol) in dioxane (5.00 mL), was added Pd(dppf)Cl₂ (15.00 mg) under N₂ atmosphere. The reaction mixture was stirred at 90° C. for 2 h. The solvent was removed. The residue was purified by pre-TLC pre-HPLC to give the desired product (15.84 mg, yield: 15%) as a white solid. ¹H NMR (400 MHz, d₆-DMSO): δ 7.84 (d, J=5.6 Hz, 1H), 7.71 (s, 1H), 7.59 (d, J=8.4 Hz, 1H), 7.28-7.33 (m, 3H), 6.86 (dd, J=5.2 Hz, 1.6 Hz 1H), 6.76 (s, 1H), 5.90 (s, 2H), 3.99 (d, J=7.2 Hz, 2H), 3.15-3.20 (m, 2H), 2.88 (t, J=8.0 Hz, 2H), 2.63 (s, 6H), 2.12-2.15 (m, 1H), 0.86 (d, J=6.4 Hz, 6H). LCMS (Method B), RT 1.94 min, calcd. for C₂₁H₂₉N₅O₂S 415.20 m/z, found 416.3 m/z [M+H]⁺.

### Example B-230: ({2-[1-(butan-2-yl)-6-(pyridin-4-yl)-1H-indol-3-yl]ethyl}sulfamoyl)dimethylamine

### Step 1. 6-bromo-1-sec-butyl-1H-indole

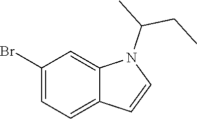

To a stirred solution of 6-bromoindole (4.5 g, 23.07 mmol) and 2-bromobutane (4.71 g, 34.62 mmol) in DMF (20 mL), was added K₂CO₃ (6.37 g, 46.14 mmol). The resulting reaction mixture was stirred at 80° C. for 12 h and cooled to room temperature. Water (100 mL) and EA (100 mL) was added. The organic layer was separated. The aqueous layer was extracted with EA (2×50 mL). The combined organic layer was washed with brine (2×50 mL), dried over anhydrous Na₂SO₄ and evaporated to give crude product which was purified by prep-TLC to give the desired product as a yellow solid (1.0 g, yield: 17%).

### Step 2. 2-(6-bromo-1-sec-butyl-1H-indol-3-yl)-2-oxoacetamide

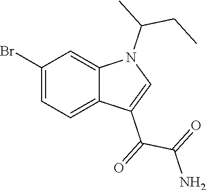 [SMILES: CCC(C)n1cc(C(=O)C(N)=O)c2ccc(Br)cc21]

To a stirred solution of 6-bromo-1-sec-butyl-1H-indole (1.0 g, 3.98 mmol) in dichloromethane (10 ml) was added oxalyl chloride (0.75 g, 5.97 mmol) slowly at 0° C. The resulting reaction mixture was allowed to warm to room temperature, stirred at rt for 30 min and then poured into 10% aq. ammonium hydroxide. The solid was collected by filtration, washed with water and dried to give the product as a white solid (0.70 g, yield: 55%).

### Step 3. 2-(6-bromo-1-sec-butyl-1H-indol-3-yl)ethanamine

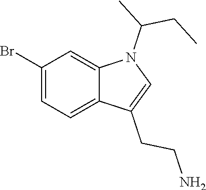 [SMILES: CCC(C)n1cc(CCN)c2ccc(Br)cc21]

To 2-(6-bromo-1-sec-butyl-1H-indol-3-yl)-2-oxoacetamide (700 mg, 2.17 mmol) was added BH₃ in THF (1 N, 5 mL). The solution was stirred at room temperature for 12 h. Conc. HCl (1 mL) was added. The reaction mixture was stirred for another 10 min. The solvent was removed under reduced pressure. The residue was purified by prep-HPLC to give the product as a yellow oil (550 mg, yield: 86%).

### Step 4. ({2-[6-bromo-1-(butan-2-yl)-1H-indol-3-yl]ethyl}sulfamoyl)dimethylamine

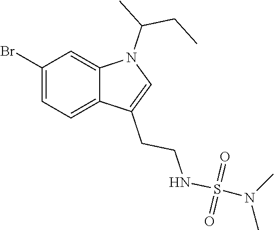 [SMILES: CCC(C)n1cc(CCNS(=O)(=O)N(C)C)c2ccc(Br)cc21]

To a stirred solution of 2-(6-bromo-1-sec-butyl-1H-indol-3-yl)ethanamine (550 mg, 1.87 mmol) and TEA (567 mg, 5.61 mmol) in DCM (10 mL), was added dimethylsulfamoyl chloride (401 mg, 2.81 mmol) dropwise at room temperature. The mixture was stirred for 12 h and the solvent was removed under reduced pressure. The residue was purified by prep-HPLC to give the product as a yellow oil (100 mg, yield: 13%).

### Step 5. ({2-[1-(butan-2-yl)-6-(pyridin-4-yl)-1H-indol-3-yl]ethyl}sulfamoyl)dimethylamine

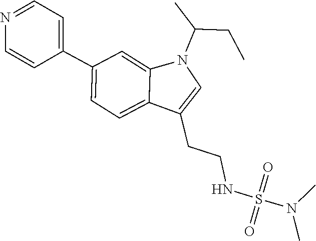 [SMILES: CCC(C)n1cc(CCNS(=O)(=O)N(C)C)c2ccc(-c3ccncc3)cc21]

To a solution of ({2-[6-bromo-1-(butan-2-yl)-1H-indol-3-yl]ethyl}sulfamoyl)dimethylamine (100 mg, 0.25 mmol), K₃PO₄·7H₂O (254 mg, 0.75 mmol) and 4-(4,4,5,5-tetramethyl-1,3,2-dioxaborolan-2-yl)pyridined (61.5 mg, 0.30 μmol) in dioxane (50 mL), was added Pd(dppf)Cl₂ (15 mg) under N₂ atmosphere. The reaction mixture was stirred at 90° C. for 2 h and cooled to room temperature. The resulting reaction was concentrated under reduced pressure, and then water (30 mL) was added. The mixture was extracted with EA (2×20 mL). The combined organic layers were washed with brine (2×50 mL), dried over Na₂SO₄, filtered, and concentrated under reduced pressure. The residue was purified by prep-HPLC to give the product as a yellow solid. (25 mg, yield: 25%). ¹H NMR (400 MHz, d₆-DMSO) δ 8.59 (d, J=6.0 Hz, 2H), 7.97 (s, 1H), 7.79 (d, J=6.4 Hz, 2H), 7.62 (d, J=8.4 Hz, 1H), 7.49-7.44 (m, 2H), 7.33 (t, J=6.0 Hz, 1H), 4.67 (d, J=6.8 Hz, 1H), 3.15-3.43 (m, 2H), 2.90 (t, J=7.6 Hz, 2H), 2.63 (s, 6H), 1.57-1.90 (m, 2H), 1.44 (d, J=6.8 Hz, 3H), 0.74 (t, J=7.2 Hz, 3H). LCMS (Method A) RT 2.15 min, calcd. for C₂₁H₂₈N₄O₂S 400.19 m/z, found 401.2 m/z [M+H]⁺.

### Example B-231: dimethyl({2-[1-(3-methylbutyl)-6-(pyridin-4-yl)-1H-indol-3-yl]ethyl}sulfamoyl)amine

### Step 1. 6-bromo-1-isopentyl-1H-indole

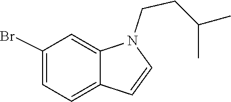

To a stirred solution of 6-bromoindole (1 g, 5.13 mmol) and 1-bromo-3-methylbutane (1.53 g, 10.26 mmol) in DMF (10 mL), was added K₂CO₃ (1.42 g, 10.26 mmol). The resulting reaction mixture was stirred at 80° C. for 12 h and cooled to room temperature. Water (50 mL) and EA (50 mL) was added. The organic layer was separated. The aqueous layer was extracted with EA (2×30 mL). The combined organic layer was washed with brine (2×30 mL), dried over anhydrous Na₂SO₄ and evaporated to give crude product which was purified by silica gel column chromatography to give the desired product as a yellow solid (400 mg, yield: 29%).

### Step 2. 2-(6-bromo-1-isopentyl-1H-indol-3-yl)-2-oxoacetamide

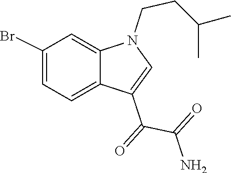 [SMILES: CC(C)CCn1cc(C(=O)C(N)=O)c2ccc(Br)cc21]

To a stirred solution of 6-bromo-1-isopentyl-1H-indole (400 mg, 1.51 mmol) in dichloromethane (10 ml) was added oxalyl chloride (285 mg, 2.26 mmol) slowly at 0° C. The resulting reaction mixture was allowed to warm to room temperature, stirred at room temperature for 1 h and then poured into 10% aq. ammonium hydroxide. The solid was collected by filtration, washed with water and dried to give the product as a white solid (500 mg, yield: 99%).

### Step 3. 2-(6-bromo-1-isopentyl-1H-indol-3-yl)ethanamine

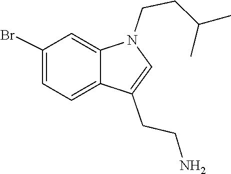 [SMILES: CC(C)CCn1cc(CCN)c2ccc(Br)cc21]

To 2-(6-bromo-1-isopentyl-1H-indol-3-yl)-2-oxoacetamide (500 mg, 1.49 mmol) was added borane-THF (1 M in THF, 5 mL). The solution was stirred at room temperature for 12 h. Conc. HCl (1 mL) was added. The reaction mixture was stirred for another 10 min. The solvent was removed under reduced pressure. The residue was purified by prep-HPLC to give the product as a yellow oil (400 mg, yield: 87%).

### Step 4. ({2-[6-bromo-1-(3-methylbutyl)-1H-indol-3-yl]ethyl}sulfamoyl)dimethylamine

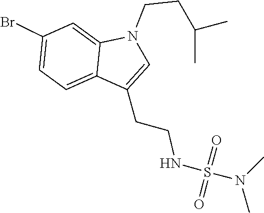 [SMILES: CC(C)CCn1cc(CCNS(=O)(=O)N(C)C)c2ccc(Br)cc21]

To a stirred solution of compound 2-(6-bromo-1-isopentyl-1H-indol-3-yl)ethanamine (400 mg, 1.30 mmol) and TEA (394 mg, 3.90 mmol) in DCM (10 mL), was added dimethylsulfamoyl chloride (279 mg, 1.95 mmol) dropwise at room temperature. The mixture was stirred for 24 h and the solvent was removed under reduced pressure. The residue was purified by prep-HPLC to give the product as a yellow oil (77 mg, yield: 14%).

### Step 5. dimethyl({2-[1-(3-methylbutyl)-6-(pyridin-4-yl)-1H-indol-3-yl]ethyl}sulfamoyl)amine

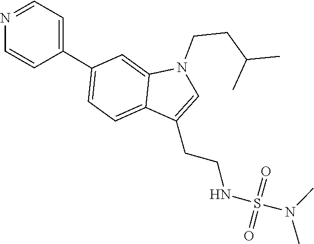 [SMILES: CC(C)CCn1cc(CCNS(=O)(=O)N(C)C)c2ccc(-c3ccncc3)cc21]

To a solution of ({2-[6-bromo-1-(3-methylbutyl)-1H-indol-3-yl]ethyl}sulfamoyl)dimethylamine (77 mg, 0.19 mmol), K₃PO₄·7H₂O (188 mg, 0.56 mmol) and 4-(4,4,5,5-tetramethyl-1,3,2-dioxaborolan-2-yl)pyridined (46.7 mg, 0.23 mmol) in dioxane (5 mL), was added Pd(dppf)Cl₂ (10 mg) under N₂ atmosphere. The reaction mixture was stirred at 90° C. for 5 h and cooled to room temperature. The resulting reaction was concentrated under reduced pressure, and then water (30 mL) was added. The mixture was extracted with EA (2×20 mL). The combined organic layers were washed with brine (2×50 mL), dried over Na₂SO₄, filtered, and concentrated under reduced pressure. The residue was purified by prep-HPLC to give the product as a yellow solid (25 mg, yield: 33%) as a yellow solid. ¹H NMR (400 MHz, d₆-DMSO) δ 8.60 (d, J=5.6 Hz, 2H), 7.90 (d, J=7.6 Hz, 1H), 7.78 (d, J=5.2 Hz, 2H), 7.61-7.66 (m, 1H), 7.46-7.52 (m, 1H), 7.25-7.40 (m, 2H), 4.19-4.32 (m, 2H), 3.18 (s, 2H), 2.85-2.95 (m, 2H), 2.64 (s, 3H), 2.63 (s, 3H), 1.62-1.72 (m, 2H), 1.45-1.59 (m, 1H), 0.90-0.99 (m, 6H). LCMS (Method A) RT 2.263 min, calcd. for C₂₂H₃₀N₄O₂S 414.21 m/z, found 415.3 m/z [M+H]⁺.

### Example B-232: dimethyl({2-[1-(2-methylpropyl)-5-(pyridin-4-yl)-1H-indol-3-yl]ethyl}sulfamoyl)amine

### Step 1. 5-bromo-1-isobutyl-1H-indole

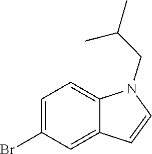

To a stirred solution of 5-bromoindole (2 g, 10.2 mmol) in DMF (40 mL) were added 1-bromo-2-methylpropane (8.4 g, 61.2 mmol) and K₂CO₃ (8.5 g, 61.2 mmol). The resulting reaction mixture was stirred at 80° C. for 12 h and cooled to room temperature. Water (250 mL) and EA (250 mL) was added. The organic layer was separated. The aqueous layer was extracted with EA (2×250 mL). The combined organic layer was washed with brine (2×250 mL), dried over anhydrous Na₂SO₄ and evaporated to give crude product which was purified by prep-HPLC to give the product as a yellow oil (700 mg, yield: 27%).

### Step 2. 2-(5-bromo-1-isobutyl-1H-indol-3-yl)-2-oxoacetamide

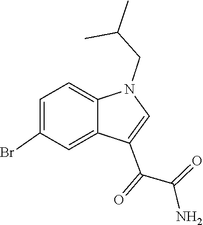 [SMILES: CC(C)Cn1cc(C(=O)C(N)=O)c2cc(Br)ccc21]

To a stirred solution of 5-bromo-1-isobutyl-1H-indole (500 mg, 1.98 mmol) in dichloromethane (10 ml), was added oxalyl chloride (4.4 g, 34.5 mmol) slowly at 0° C. The resulting reaction mixture was allowed to warm to room temperature, stirred for 1 h and then poured into 10% aq. ammonium hydroxide. The solid was collected by filtration, washed with water and dried to give the product as a yellow solid (450 mg, yield: 70%).

### Step 3. 2-(5-bromo-1-isobutyl-1H-indol-3-yl)ethanamine

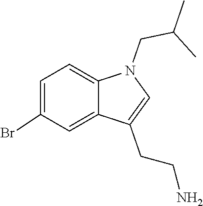 [SMILES: CC(C)Cn1cc(CCN)c2cc(Br)ccc21]

To 2-(5-bromo-1-isobutyl-1H-indol-3-yl)-2-oxoacetamide (300 mg, 0.93 mmol) was added borane-THF (1 M in THF, 20 mL). The solution was stirred at 65° C. for 2 h. Conc. HCl (2 mL, 12 N) was added. The reaction mixture was stirred for another 10 min at room temperature. The solvent was removed under reduced pressure. The residue was purified by prep-HPLC to give the product as a yellow solid (260 mg, yield: 68%).

### Step 4. ({2-[5-bromo-1-(2-methylpropyl)-1H-indol-3-yl]ethyl}sulfamoyl)dimethylamine

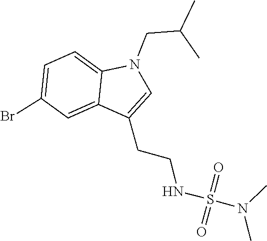 [SMILES: CC(C)Cn1cc(CCNS(=O)(=O)N(C)C)c2cc(Br)ccc21]

To a stirred solution of 2-(5-bromo-1-isobutyl-1H-indol-3-yl)ethanamine (260 mg, 0.88 mmol) and DIPEA (800 mg, 6.2 mmol) in DCM (10 mL), was added dimethylsulfamoyl chloride (600 mg, 4.2 mmol) dropwise at room temperature. The mixture was stirred for 12 h and the solvent was removed under reduced pressure. The residue was purified by prep-HPLC to give the product as a yellow oil (70 mg, yield: 20%).

### Step 5. dimethyl({2-[1-(2-methylpropyl)-5-(pyridin-4-yl)-1H-indol-3-yl]ethyl}sulfamoyl)amine

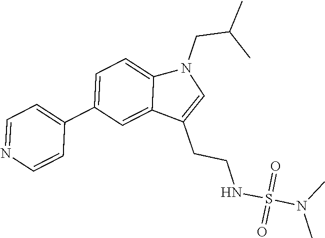 [SMILES: CC(C)Cn1cc(CCNS(=O)(=O)N(C)C)c2cc(-c3ccncc3)ccc21]

To a stirred suspension of ({2-[5-bromo-1-(2-methylpropyl)-1H-indol-3-yl]ethyl}sulfamoyl)dimethylamine (70 mg, 0.17 mmol), 4-(4,4,5,5-tetramethyl-1,3,2-dioxaborolan-2-yl)pyridine (117 mg, 0.57 mmol) and K₃PO₄·7H₂O (200 mg, 0.94 mmol) in dioxane/water (3 mL/0.3 mL), was added Pd(dppf)Cl₂ (15 mg, 0.21 mmol). The resulting reaction mixture was stirred at 90° C. for 5 h and cooled to room temperature. The solvent was removed under reduced pressure. The residue was partitioned between water (20 mL) and EA (20 mL). The organic layer was separated. The aqueous layer was extracted with EA (2×20 mL), The combined organic layer was washed with brine (2×20 mL), dried over Na₂SO₄, filtered, and concentrated under reduced pressure. The residue was purified by prep-HPLC to give the desired product as a yellow solid (60 mg, yield: 86%). ¹H NMR (400 MHz, d₆-DMSO): δ 8.59 (d, J=6.0 Hz, 2H), 8.59 (s, 1H), 7.74 (d, J=8.4 Hz, 2H), 7.57 (s, 2H), 7.26-7.35 (m, 2H), 3.96 (d, J=6.8 Hz, 2H), 3.15-3.25 (m, 2H), 2.94 (t, J=7.6 Hz, 2H), 2.62 (s, 6H), 2.05-2.18 (m, 1H), 0.86 (d, J=6.8 Hz, 6H). LCMS (Method A) RT 2.20 min, calcd. for C₂₁H₂₈N₄O₂S 400.19 m/z, found 401.3 m/z [M+H]⁺.

### Example B-233: dimethyl({2-[1-(2-methylpropyl)-6-(pyridin-4-yl)-1H-indol-3-yl]-2-oxoethyl}sulfamoyl)amine

### Step 1. ethyl 1-(6-bromo-1-isobutyl-1H-indol-3-yl)-2-chloroethanone

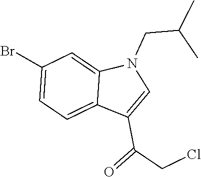 [SMILES: CC(C)Cn1cc(C(=O)CCl)c2ccc(Br)cc21]

To a solution of 6-bromo-1-isobutyl-1H-indole (1 g, 3.97 mmol) and 2-chloroacetyl chloride (671.88 mg, 5.95 mmol) in DCM (15 mL) in an ice-water bath, was added AlCl₃ (581.69 mg, 4.36 mmol) in five portions. The mixture was stirred at room temperature for 2 h. K₂CO₃ (1.0 N) was added until pH>8. The organic layer was separated. The aqueous was extracted with DCM (2×30 mL). The combined organic layers were washed with brine (30.0 mL), dried over anhydrous Na₂SO₄, filtered and concentrated in vacuo to give crude product, which was washed with petroleum ether to give the product as a pink solid (800 mg, yield: 61%).

### Step 2. 2-amino-1-(6-bromo-1-isobutyl-1H-indol-3-yl)ethanone

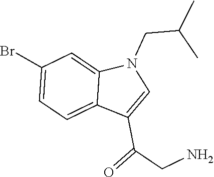 [SMILES: CC(C)Cn1cc(C(=O)CN)c2ccc(Br)cc21]

Ethyl 1-(6-bromo-1-isobutyl-1H-indol-3-yl)-2-chloroethanone (150 mg, 456.44 μmol) was dissolved in acetone (5 mL). A solution of ammonia in THF (1 N, 2 mL) was added, and the reaction mixture was refluxed for 12 hr. The solvent was removed under reduced pressure. The residue was purified by prep-HPLC to give the product as a white solid (75 mg, yield: 53%).

### Step 3. ({2-[6-bromo-1-(2-methylpropyl)-1H-indol-3-yl]-2-oxoethyl}sulfamoyl)dimethylamine

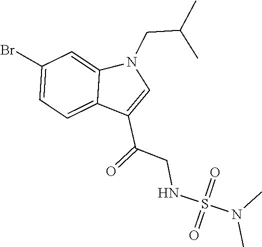 [SMILES: CC(C)Cn1cc(C(=O)CNS(=O)(=O)N(C)C)c2ccc(Br)cc21]

To a solution of 2-amino-1-(6-bromo-1-isobutyl-1H-indol-3-yl)ethanone (75 mg, 242.56 μmol) and DIEA (93.87 mg, 727.68 μmol) in THF (5 mL), was added N,N-dimethylsulfamoyl chloride (34.83 mg, 242.56 μmol). The mixture was stirred at room temperature for 12 hr. The solvent was removed under reduced pressure. The residue was purified by prep-TLC to give the product (35 mg, yield: 34%) as a yellow solid.

### Step 4. dimethyl({2-[1-(2-methylpropyl)-6-(pyridin-4-yl)-1H-indol-3-yl]-2-oxoethyl}sulfamoyl)amine

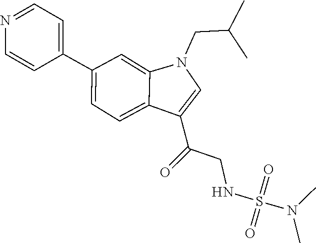 [SMILES: CC(C)Cn1cc(C(=O)CNS(=O)(=O)N(C)C)c2ccc(-c3ccncc3)cc21]

To a solution of ({2-[6-bromo-1-(2-methylpropyl)-1H-indol-3-yl]-2-oxoethyl}sulfamoyl)dimethylamine (120 mg, 288.23 μmol), 4-(4,4,5,5-tetramethyl-1,3,2-dioxaborolan-2-yl)pyridine (59.10 mg, 288.23 μmol) and K₃PO₄·7H₂O (292.27 mg, 864.69 μmol) in dioxane (5 mL), was added Pd(dppf)Cl₂ (21.10 mg, 28.82 μmol) under N₂ atmosphere. The reaction mixture stirred at 90° C. for 5 h. The solvent was removed under reduced pressure. The residue was partitioned between EA (25.0 mL) and water (25.0 mL). The organic layer was separated. The aqueous layer was extracted with EA (2×30.0 mL). The combined organic layer was washed with brine (2×30 mL), dried over Na₂SO₄, filtered, and concentrated under reduced pressure. The residue was purified by prep-HPLC to give the product as a white solid (38 mg, yield: 31%). ¹H NMR (400 MHz, d₆-DMSO): δ 8.64-8.65 (m, 2H), 8.60 (s, 1H), 8.30 (d, J=8.4 Hz, 1H), 8.13 (s, 1H), 7.82-7.84 (m, 2H), 7.70-7.72 (m, 1H), 7.48 (t, J=5.6 Hz, 1H), 4.37 (d, J=9.6 Hz, 2H), 4.18 (d, J=7.2 Hz, 2H), 2.69 (s, 6H), 2.22-2.33 (m, 1H), 0.91 (d, J=6.8 Hz, 6H). LCMS (Method A) RT 1.912 min, calcd. for C₂₁H₂₆N₄O₃S 414.17 m/z, found 415.4 m/z [M+H]⁺.

### Example B-234: N-(dimethylsulfamoyl)-2-[1-(2-methylpropyl)-6-(pyridin-4-yl)-1H-indol-3-yl]acetamide

### Step 1. 2-(6-bromo-1-isobutyl-1H-indol-3-yl)-2-hydroxyacetamide

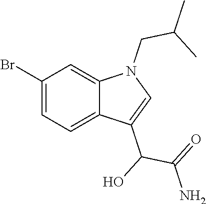 [SMILES: CC(C)Cn1cc(C(O)C(N)=O)c2ccc(Br)cc21]

To a suspension of 2-(6-bromo-1-isobutyl-1H-indol-3-yl)-2-oxoacetamide (1.5 g, 4.64 mmol) in MeOH (10 mL) in an ice-water bath was added NaBH₄ (176 mg, 1.03 mmol) in portions. The mixture was allowed to warm to room and stirred for 4 h. 1.0 N HCl was added until pH<5. The solvent was removed under reduced pressure. The residue was purified by prep-HPLC to give the product as a white solid (1.3 g, yield: 86%).

### Step 2. 2-(6-bromo-1-isobutyl-1H-indol-3-yl)acetamide

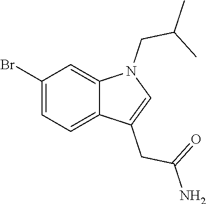 [SMILES: CC(C)Cn1cc(CC(N)=O)c2ccc(Br)cc21]

To a stirred solution of 2-(6-bromo-1-isobutyl-1H-indol-3-yl)-2-hydroxyacetamide (1.3 g, 4.00 mmol) in MeCN (10 ml), were added TMSCl (1.3 g, 11.99 mmol) and NaI (1.8 g, 11.99 mmol) slowly at 0° C. The mixture was allowed to warm to room and stirred for another 2 h and cooled down to 0° C., Ac₂O (1.22 g, 11.99 mmol) was added dropwise slowly. The mixture was stirred for another 3 h and then poured into ice-water. The solvent was removed under reduced pressure. The residue was purified by prep-HPLC to give the product as a green oil (700 mg, yield: 57%).

### Step 3. 2-(6-bromo-1-isobutyl-1H-indol-3-yl)acetic acid

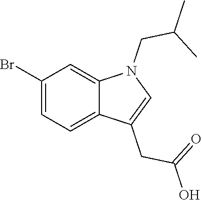 [SMILES: CC(C)Cn1cc(CC(=O)O)c2ccc(Br)cc21]

To a mixture of 2-(6-bromo-1-isobutyl-1H-indol-3-yl)acetamide (700 mg, 2.26 mmol) in dioxane (10 mL), was added HCl (10 mL, 12 N). The reaction mixture was stirred at 80° C. for 1 h. The solvent was removed under reduced pressure. The residue was purified by prep-HPLC to give the product as a yellow oil (350 mg, yield: 49%).

### Step 4. 2-(6-bromo-1-isobutyl-1H-indol-3-yl)-N—(N,N-dimethylsulfamoyl)acetamide

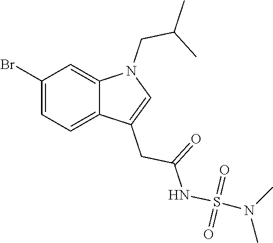 [SMILES: CC(C)Cn1cc(CC(=O)NS(=O)(=O)N(C)C)c2ccc(Br)cc21]

To a stirred solution of 2-(6-bromo-1-isobutyl-1H-indol-3-yl)acetic acid (350 mg, 1.13 mmol), [methyl(sulfamoyl)amino]methane (350 mg, 2.82 mmol) and DIPEA (742 mg, 5.74 mmol) in DCM (5 mL), was added TBTU (550 mg, 1.71 mmol) in portions at room temperature. The mixture was stirred for 12 h and the solvent was removed under reduced pressure. The residue was purified by prep-HPLC to give the product as a yellow oil (160 mg, yield: 34%).

### Step 5. N-(dimethylsulfamoyl)-2-[1-(2-methylpropyl)-6-(pyridin-4-yl)-1H-indol-3-yl]acetamide

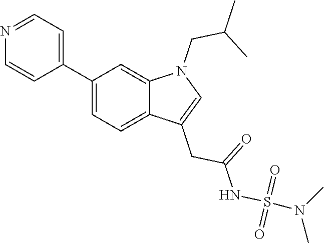 [SMILES: CC(C)Cn1cc(CC(=O)NS(=O)(=O)N(C)C)c2ccc(-c3ccncc3)cc21]

To a suspension of 2-(6-bromo-1-isobutyl-1H-indol-3-yl)-N—(N,N-dimethylsulfamoyl)acetamide (160 mg, 384 μmol), 4-(4,4,5,5-tetramethyl-1,3,2-dioxaborolan-2-yl)pyridine (160 mg, 780 μmol) and K₃PO₄·7H₂O (350 mg, 1.65 mmol) in dioxane/water (3 mL/0.3 mL), was added Pd(dppf)Cl₂ (40 mg, 55 μmol) under N₂ atmosphere. The reaction mixture was stirred at 90° C. for 5 h. The resulting reaction was concentrated under reduced pressure, and then water (15.0 mL) was added. The mixture was extracted with EA (2×15 mL). The combined organic layer was washed with brine (15 mL), dried over anhydrous Na₂SO₄, filtered, and concentrated under reduced pressure. The residue was purified by prep-HPLC to give the product as a yellow solid (31 mg, yield: 19%). ¹H NMR (400 MHz, d₆-DMSO): δ 11.62 (s, 1H), 8.60 (d, J=1.6 Hz, 2H), 7.95 (s, 1H), 7.79 (d, J=6.4 Hz, 2H), 7.65 (d, J=8.4 Hz, 1H), 7.49 (d, J=8.4 Hz, 1H), 7.36 (s, 1H), 4.07 (d, J=7.2 Hz, 2H), 3.71 (s, 2H), 2.75 (s, 6H), 2.10-2.21 (m, 1H), 0.87 (d, J=6.8 Hz, 6H). LCMS (Method A) RT 1.599 min, calcd. for C₂₁H₂₆N₄O₃S 414.17 m/z, found 415.3 m/z [M+H]⁺.

### Example B-235: dimethyl({3-[1-(2-methylpropyl)-6-(pyridin-4-yl)-1H-indol-3-yl]propyl}sulfamoyl)amine

### Step 1. 2-(6-bromo-1-isobutyl-1H-indol-3-yl)-N-(isopropylsulfonyl)acetamide

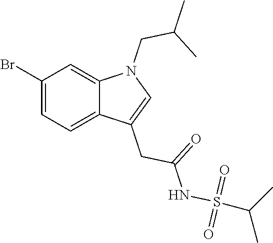 [SMILES: CC(C)Cn1cc(CC(=O)NS(=O)(=O)C(C)C)c2ccc(Br)cc21]

To a stirred solution of 2-(6-bromo-1-isobutyl-1H-indol-3-yl)acetic acid (210 mg, 0.68 mmol), propane-2-sulfonamide (198 mg, 1.61 mmol) and DIPEA (400 mg, 0.54 mmol) in DCM (3 mL), was added TBTU (450 mg, 1.40 mmol) at room temperature. The mixture was stirred for 12 h and the solvent was removed under reduced pressure. The residue was purified by prep-HPLC to give the product as a yellow oil (120 mg, yield: 43%).

### Step 2. dimethyl({3-[1-(2-methylpropyl)-6-(pyridin-4-yl)-1H-indol-3-yl]propyl}sulfamoyl)amine

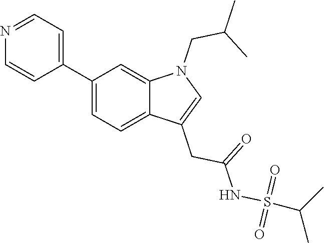 [SMILES: CC(C)Cn1cc(CC(=O)NS(=O)(=O)C(C)C)c2ccc(-c3ccncc3)cc21]

To a suspension of 2-(6-bromo-1-isobutyl-1H-indol-3-yl)-N-(isopropylsulfonyl)acetamide (50 mg, 120 μmol), 4-(4,4,5,5-tetramethyl-1,3,2-dioxaborolan-2-yl)pyridine (50 mg, 244 μmol) and K₃PO₄·7H₂O (100 mg, 471 μmol) in dioxane/water (3 mL/0.3 mL), was added Pd(dppf)Cl₂ (20 mg, 27 μmol) under N₂ atmosphere. The reaction mixture was stirred at 90° C. for 5 hr. The resulting reaction was concentrated under reduced pressure, and then water (15.0 mL) was added. The mixture was extracted with EA (2×15 mL). The combined organic layer was washed with brine (15 mL), dried over anhydrous Na₂SO₄, filtered, and concentrated under reduced pressure. The residue was purified by prep-HPLC to give the product as a yellow solid (26 mg, yield: 52%). ¹H NMR (400 MHz, d₆-DMSO): δ 11.75 (s, 1H), 8.58 (d, J=6.0 Hz, 2H), 7.92 (s, 1H), 7.76 (d, J=7.2 Hz, 2H), 7.62 (d, J=8.4 Hz, 1H), 7.46 (d, J=8.4 Hz, 1H), 7.34 (s, 1H), 4.04 (d, J=7.2 Hz, 2H), 3.71 (s, 2H), 3.48-3.59 (m, 1H), 2.06-2.16 (m, 1H), 1.19 (d, J=6.8 Hz, 6H), 0.84 (d, J=6.4 Hz, 6H). LCMS (Method A) RT 1.494 min, calcd. for C₂₂H₂₇N₃O₃S 413.18 m/z, found 414.4 m/z [M+H]⁺.

### Example B-236: dimethyl({2-[2-methyl-1-(2-methylpropyl)-6-(pyridin-4-yl)-1H-indol-3-yl]ethyl}sulfamoyl)amine

### Step 1. 1-(6-bromo-1-isobutyl-1H-indol-3-yl)-3-chloropropan-1-one

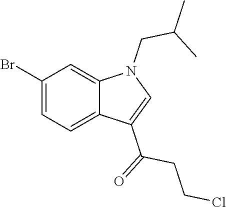 [SMILES: CC(C)Cn1cc(C(=O)CCCl)c2ccc(Br)cc21]

To a solution of 6-bromo-1-isobutyl-1H-indole (1 g, 3.97 mmol) and 3-chloropropanoyl chloride ((755.32 mg, 5.95 mmol) in DCM (15 mL) in an ice-water bath, was added AlCl₃ (581.70 mg, 4.36 mmol) in portions. The mixture was stirred at room temperature for 2 h. K₂CO₃ (1.0 N) was added until pH>8. The organic layer was separated. The aqueous was extracted with DCM (2×30 mL). The combined organic layer was washed with brine (30.0 mL), dried over anhydrous Na₂SO₄, filtered and concentrated in vacuo to give crude product which was washed with petrol to give the product as a white solid (600 mg, yield: 44%).

### Step 2. 6-bromo-3-(3-chloropropyl)-1-isobutyl-1H-indole

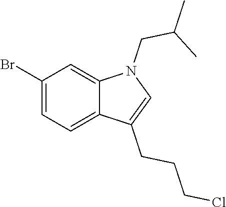 [SMILES: CC(C)Cn1cc(CCCCl)c2ccc(Br)cc21]

A mixture of compound 1-(6-bromo-1-isobutyl-1H-indol-3-yl)-3-chloropropan-1-one (600 mg, 1.75 mmol) and borane-THF (1.0 N in THF, 3.5 mL) was stirred for 3 h at room temperature. Aq. HCl (6.0 N, 2 mL) was added. The mixture was stirred for 10 min. The solvent was removed under reduced pressure. The residue was purified by prep-HPLC to give the product as colorless oil (300 mg, yield: 52%).

### Step 3. 3-(6-bromo-1-isobutyl-1H-indol-3-yl)propan-1-amine

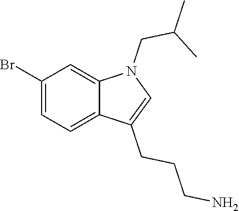 [SMILES: CC(C)Cn1cc(CCCN)c2ccc(Br)cc21]

A mixture of. 6-bromo-3-(3-chloropropyl)-1-isobutyl-1H-indole (250 mg, 760.63 μmol) and NaI (11.40 mg, 76.06 μmol) in NH₃ in methanol (1 M, 3.80 mL) was stirred in sealed tube at 75° C. for 24 hr. The solvent was removed under reduced pressure. The residue was purified by prep-HPLC to give the product as a white solid (120 mg, yield: 51%).

### Step 4. ({3-[6-bromo-1-(2-methylpropyl)-1H-indol-3-yl]propyl}sulfamoyl)dimethylamine

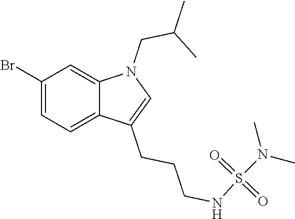 [SMILES: CC(C)Cn1cc(CCCNS(=O)(=O)N(C)C)c2ccc(Br)cc21]

To a solution of 3-(6-bromo-1-isobutyl-1H-indol-3-yl)propan-1-amine (120 mg, 388.04 μmol) and DIEA (150.17 mg, 1.16 mmol) in THF (5 mL), was added dimethylsulfamoyl chloride (83.58 mg, 582.06 μmol). The mixture was stirred at 50° C. overnight. The solvent was removed under reduced pressure. The residue was purified by prep-HPLC to give the product as a white solid (100 mg, yield: 61%).

### Step 5. dimethyl({2-[2-methyl-1-(2-methylpropyl)-6-(pyridin-4-yl)-1H-indol-3-yl]ethyl}sulfamoyl)amine

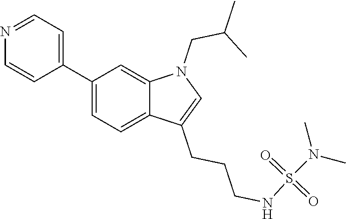 [SMILES: CC(C)Cn1cc(CCCNS(=O)(=O)N(C)C)c2ccc(-c3ccncc3)cc21]

To a solution of ({3-[6-bromo-1-(2-methylpropyl)-1H-indol-3-yl]propyl}sulfamoyl)dimethylamine (100 mg, 240.17 μmol), 4-(4,4,5,5-tetramethyl-1,3,2-dioxaborolan-2-yl)pyridine (59.10 mg, 288.23 μmol) and K₃PO₄·7H₂O (243.53 mg, 720.50 μmol) in dioxane (5 mL) was added Pd(dppf)Cl₂ (17.58 mg, 24.02 μmol) under N₂ atmosphere. The reaction mixture stirred at 90° C. for 5 hr and cooled to room temperature. The solvent was removed under reduced pressure. The residue was partitioned between EA (25.0 mL) and water (25.0 mL). The organic layer was separated. The aqueous layer was extracted with EA (2×30.0 mL). The combined organic layer was washed with brine (2×30 mL), dried over Na₂SO₄, filtered, and concentrated under reduced pressure. The residue was purified by prep-HPLC to give the product as a yellow solid (33.91 mg, yield: 34%). ¹H NMR (400 MHz, d₆-DMSO): δ 8.59-8.61 (m, 2H), 7.92 (s, 1H), 7.78-7.80 (m, 2H), 7.65 (d, J=8.4 Hz, 1H), 7.44-7.46 (m, 1H), 7.21-7.26 (m, 2H), 4.03 (d, J=7.2 Hz, 2H), 2.92-2.97 (m, 2H), 2.75 (t, J=7.6 Hz, 2H), 2.64 (s, 6H), 2.13-2.16 (m, 1H), 1.78-2.1.86 (m, 2H), 0.86 (d, J=6.4 Hz, 6H). LCMS (Method A) RT 2.061 min, calcd. for C₂₂H₃₀N₄O₂S 414.21 m/z, found 415.2 m/z [M+H]⁺.

### Example B-237: [(2-{1-[2-(tert-butoxy)ethyl]-6-(pyridin-4-yl)-1H-indol-3-yl}ethyl)sulfamoyl]dimethylamine

### Step 1. 6-bromo-1-sec-butyl-2-methyl-1H-indole

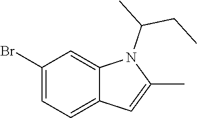

To a solution of 6-bromo-2-methylindole (3 g, 14.28 mmol) and 1-bromo-2-methyl-propane (12.50 g, 91.23 mmol) in DMF (12 mL), were added K₂CO₃ (10.00 g, 30.69 mmol) and NaI (1 g, 6.67 mmol) in portions. The mixture was stirred at 80° C. for 12 h and cooled to room temperature. Water (100 mL) and EA (50 mL) was added. The organic layer was separated. The aqueous layer was extracted with EA (2×50 mL). The combined organic layer was washed with brine (50 mL), dried over anhydrous Na₂SO₄ and evaporated to give the product as a yellow oil (1.6 g, yield: 42%).

### Step 2. 2-(6-bromo-1-sec-butyl-2-methyl-1H-indol-3-yl)-2-oxoacetamide

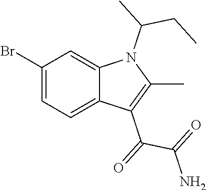 [SMILES: CCC(C)n1c(C)c(C(=O)C(N)=O)c2ccc(Br)cc21]

To a stirred solution of 6-bromo-1-sec-butyl-2-methyl-1H-indole (1.6 g, 6.01 mmol) in dichloromethane (10 ml) was added oxalyl chloride (2.92 g, 23.01 mmol) slowly at 0° C. The resulting reaction mixture was allowed to warm to room temperature, stirred for 1 h and then poured into 10% aq. ammonium hydroxide. The solid was collected by filtration, washed with water and dried to give the product as a yellow oil (1.1 g, yield: 54%).

### Step 3. 2-(6-bromo-1-sec-butyl-2-methyl-1H-indol-3-yl)ethanamine

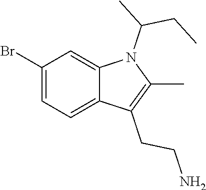 [SMILES: CCC(C)n1c(C)c(CCN)c2ccc(Br)cc21]

The solution of 2-(6-bromo-1-sec-butyl-2-methyl-1H-indol-3-yl)-2-oxoacetamide (1.1 g, 3.26 mmol) and borane-THF (1 N in THF, 15 mL) was stirred at room temperature for 12 h. Conc. HCl (1 mL) was added. The reaction mixture was stirred for another 10 min. The solvent was removed under reduced pressure. The residue was purified by prep-HPLC to give the product as a white solid (550 mg, yield: 54%).

### Step 4. ({2-[6-bromo-1-(butan-2-yl)-2-methyl-1H-indol-3-yl]ethyl}sulfamoyl)dimethylamine

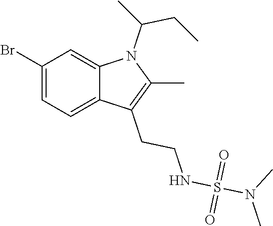 [SMILES: CCC(C)n1c(C)c(CCNS(=O)(=O)N(C)C)c2ccc(Br)cc21]

To a stirred solution of 2-(6-bromo-1-sec-butyl-2-methyl-1H-indol-3-yl)ethanamine (550 mg, 1.78 mmol) and DIPEA (1.11 g, 8.61 mmol) in DCM (5 mL), was added dimethylsulfamoyl chloride (800 mg, 5.57 mmol) dropwise at room temperature. The mixture was stirred for 18 h and then the solvent was removed under reduced pressure. The residue was purified by prep-HPLC to give the product as a yellow solid (80 mg, yield: 10%).

### Step 5. [(2-{1-[2-(tert-butoxy)ethyl]-6-(pyridin-4-yl)-1H-indol-3-yl}ethyl)sulfamoyl]dimethylamine

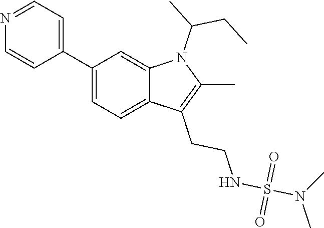 [SMILES: CCC(C)n1c(C)c(CCNS(=O)(=O)N(C)C)c2ccc(-c3ccncc3)cc21]

To a suspension of ({2-[6-bromo-1-(butan-2-yl)-2-methyl-1H-indol-3-yl]ethyl}sulfamoyl)dimethylamine (80 mg, 192 μmol), 4-(4,4,5,5-tetramethyl-1,3,2-dioxaborolan-2-yl)pyridine (80 mg, 390 μmol) and K₃PO₄·7H₂O (350 mg, 1.65 mmol) in dioxane/water (3 mL/0.3 mL), was added Pd(dppf)Cl₂ (40 mg, 55 μmol) under N₂ atmosphere. The reaction mixture was stirred at 90° C. for 2 hr. The resulting reaction was concentrated under reduced pressure, and then water (15.0 mL) was added. The mixture was extracted with EA (2×15 mL). The combined organic layer was washed with brine (15 mL), dried over anhydrous Na₂SO₄, filtered, and concentrated under reduced pressure. The residue was purified by prep-HPLC to give the product as a yellow solid (48 mg, yield: 60%). ¹H NMR (400 MHz, d₆-DMSO): δ 8.59 (d, J=6.0 Hz, 2H), 7.86 (s, 1H), 7.77 (d, J=6.0 Hz, 2H), 7.55 (d, J=8.0 Hz, 1H), 7.45 (d, J=8.4 Hz, 1H), 7.28 (t, J=1.6 Hz, 1H), 4.03 (d, J=7.2 Hz, 2H), 3.01-3.09 (m, 2H), 2.84-2.91 (m, 2H), 2.61 (s, 6H), 2.39 (s, 3H), 2.05-2.19 (m, 1H), 0.89 (d, J=6.8 Hz, 6H). LCMS (Method A) RT 2.053 min, calcd. for C₂₂H₃₀N₄O₂S 414.21 m/z, found 415.3 m/z [M+H]⁺.

### Example B-238: dimethyl({[1-(2-methylpropyl)-6-(pyridin-4-yl)-1H-indol-3-yl]methyl}sulfamoyl)amine

### Step 1. 6-bromo-1-(2-tert-butoxyethyl)-1H-indole

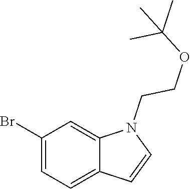 [SMILES: CC(C)(C)OCCn1ccc2ccc(Br)cc21]

To a solution of 6-bromoindole (1.5 g, 7.65 mmol) and 2-tert-butoxyethyl 4-methylbenzenesulfonate (7.5 g, 27.54 mmol) in DMF (25 mL), was added Cs₂CO₃ (21.00 g, 64.45 mmol) in portions. The mixture was stirred at 90° C. for 12 h and cooled to room temperature. Water (100 mL) and EA (150 mL) was added. The organic layer was separated. The aqueous layer was extracted with EA (2×150 mL). The combined organic layer was washed with brine (4×150 mL), dried over anhydrous Na₂SO₄ and evaporated to give the product as a colorless oil (1.4 g, yield: 61%).

### Step 2. 2-(6-bromo-1-(2-(tert-butoxy)ethyl)-1H-indol-3-yl)-2-oxoacetamide

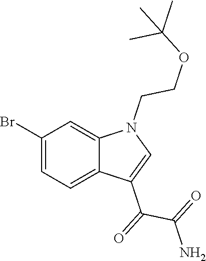 [SMILES: CC(C)(C)OCCn1cc(C(=O)C(N)=O)c2ccc(Br)cc21]

To a stirred solution of 6-bromo-1-(2-tert-butoxyethyl)-1H-indole (1.4 g, 4.73 mmol) in dichloromethane (20 ml) was added oxalyl chloride (730 mg, 5.75 mmol) slowly at 0° C. The resulting reaction mixture was allowed to warm to room temperature, stirred at room temperature for 0.5 h and then poured into 10% aq. ammonium hydroxide. The solid was collected by filtration, washed with water and dried to give the product as a yellow solid (1.5 g, yield: 86%).

### Step 3. 2-(6-bromo-1-(2-(tert-butoxy)ethyl)-1H-indol-3-yl)ethan-1-amine

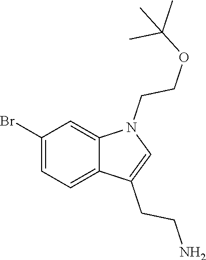 [SMILES: CC(C)(C)OCCn1cc(CCN)c2ccc(Br)cc21]

A solution of 2-(6-bromo-1-(2-(tert-butoxy)ethyl)-1H-indol-3-yl)-2-oxoacetamide (1.0 g, 2.72 mmol) in borane-THF (1 N in THF, 12 mL) was stirred at room temperature for 12 h. Conc. HCl (1 mL) was added. The reaction mixture was stirred for another 10 min. The solvent was removed under reduced pressure. The residue was purified by prep-HPLC to give the product as a white solid (400 mg, yield: 43%).

### Step 4. [(2-{6-bromo-1-[2-(tert-butoxy)ethyl]-1H-indol-3-yl}ethyl)sulfamoyl]dimethylamine

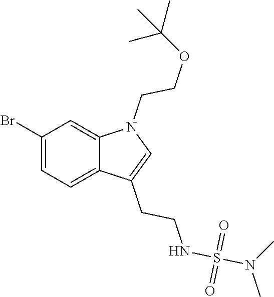 [SMILES: CN(C)S(=O)(=O)NCCc1cn(CCOC(C)(C)C)c2cc(Br)ccc12]

To a stirred solution of 2-(6-bromo-1-sec-butyl-2-methyl-1H-indol-3-yl)ethanamine (400 mg, 1.18 mmol) and DIPEA (742 mg, 5.74 mmol) in DCM (5 mL), was added dimethylsulfamoyl chloride (500 mg, 3.48 mmol) dropwise at room temperature. The mixture was stirred for 12 h and the solvent was removed under reduced pressure. The residue was purified by prep-HPLC to give the product as a yellow solid (150 mg, yield: 28%).

### Step 5. [(2-{1-[2-(tert-butoxy)ethyl]-6-(pyridin-4-yl)-1H-indol-3-yl}ethyl)sulfamoyl]dimethylamine

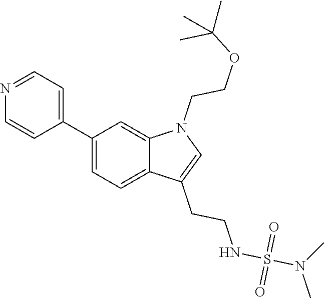 [SMILES: CN(C)S(=O)(=O)NCCc1cn(CCOC(C)(C)C)c2cc(-c3ccncc3)ccc12]

To a suspension of [(2-{6-bromo-1-[2-(tert-butoxy)ethyl]-1H-indol-3-yl}ethyl)sulfamoyl]dimethylamine (150 mg, 336 μmol), 4-(4,4,5,5-tetramethyl-1,3,2-dioxaborolan-2-yl)pyridine (150 mg, 731 μmol) and K₃PO₄·7H₂O (350 mg, 1.65 mmol) in dioxane/water (3 mL/0.3 mL), was added Pd(dppf)Cl₂ (40 mg, 55 μmol) under N₂ atmosphere. The reaction mixture was stirred at 90° C. for 2 hr. The resulting reaction was concentrated under reduced pressure, and then water (15.0 mL) was added. The mixture was extracted with EA (2×15 mL). The combined organic layer was washed with brine (15 mL), dried over anhydrous Na₂SO₄, filtered, and concentrated under reduced pressure. The residue was purified by prep-HPLC to give the product as a yellow solid (46 mg, yield: 30%). ¹H NMR (400 MHz, d₆-DMSO): δ 8.60 (d, J=4.8 Hz, 2H), 7.96 (d, J=0.8 Hz, 1H), 7.78 (d, J=4.8 Hz, 2H), 7.62 (d, J=8.4 Hz, 1H), 7.48 (d, J=8.0 Hz, 1H), 7.27-7.34 (m, 2H), 4.31 (t, J=5.6 Hz, 2H), 3.63 (t, J=5.6 Hz, 2H), 3.13-3.21 (m, 2H), 2.86-2.93 (m, 2H), 2.64 (s, 6H), 1.01 (s, 9H). LCMS (Method A) RT 1.949 min, calcd. for C₂₃H₃₂N₄O₃S 444.22 m/z, found 445.1 m/z [M+H]⁺.

### Example B-239: dimethyl({[1-(2-methylpropyl)-6-(pyridin-4-yl)-1H-indol-3-yl]methyl}sulfamoyl)amine

### Step 1. isobutyl 6-bromo-1-isobutyl-1H-indole-3-carboxylate

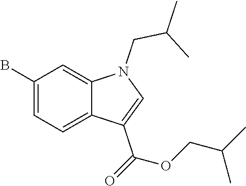

To a solution of 6-bromo-1H-indole-3-carboxylic acid (1 g, 4.17 mmol) and 1-bromo-2-methyl-propane (1.71 g, 12.50 mmol, 1.37 mL) in DMF (20 mL), was added K₂CO₃ (1.73 g, 12.50 mmol) in portions. The mixture was stirred at 80° C. overnight and cooled to room temperature. Water (100 mL) and EA (50 mL) was added. The organic layer was separated. The aqueous layer was extracted with EA (2×50 mL). The combined organic layer was washed with brine (50 mL), dried over anhydrous Na₂SO₄ and evaporated to give the product as a yellow oil (1.2 g, yield: 81%).

### Step 2. isobutyl 1-isobutyl-6-(pyridin-4-yl)-1H-indole-3-carboxylate

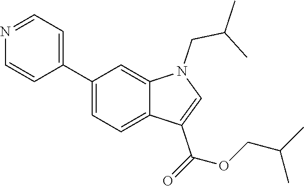 [SMILES: CC(C)COC(=O)c1cn(CC(C)C)c2cc(-c3ccncc3)ccc12]

To a solution of compound isobutyl 6-bromo-1-isobutyl-1H-indole-3-carboxylate (900 mg, 2.55 mmol), K₃PO₄·7H₂O (2.59 g, 7.65 mmol) and 4-(4,4,5,5-tetramethyl-1,3,2-dioxaborolan-2-yl)pyridine (628 mg, 3.06 mol) in dioxane (20 mL), was added Pd(dppf)Cl₂ (0.2 g) under N₂ atmosphere. The reaction mixture was stirred at 90° C. for 2 h and then cooled to room temperature. The resulting reaction was concentrated under reduced pressure, and then water (100 mL) was added. The mixture was extracted with EA (2×100 mL). The combined organic layers were washed with brine (2×50 mL), dried over Na₂SO₄, filtered, and concentrated under reduced pressure. The residue was purified by prep-HPLC to give the product as a white solid (800 mg, yield: 89%).

### Step 3. (1-isobutyl-6-(pyridin-4-yl)-1H-indol-3-yl)methanol

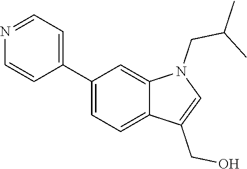 [SMILES: CC(C)Cn1cc(CO)c2ccc(-c3ccncc3)cc21]

To a solution of compound isobutyl 1-isobutyl-6-(pyridin-4-yl)-1H-indole-3-carboxylate (880 mg, 2.51 mmol) in THF (40.0 mL) in an ice-water bath, was added LAH (95.3 mg, 2.51 mmol) in portions. The reaction mixture was allowed to warm to room temperature and stirred overnight. Water (0.2 mL) and 10% NaOH (0.2 mL) were added. The solid was for filtered off. The filtrate was evaporated to give the product as a yellow solid (500 mg, yield: 71%).

### Step 4. 3-(azidomethyl)-1-isobutyl-6-(pyridin-4-yl)-1H-indole

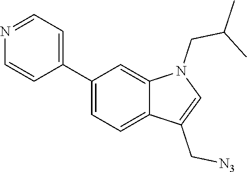 [SMILES: CC(C)Cn1cc(CN=[N+]=[N-])c2ccc(-c3ccncc3)cc21]

To a solution of (1-isobutyl-6-(pyridin-4-yl)-1H-indol-3-yl)methanol (450 mg, 1.61 mmol) and DPPA (883.42 mg, 3.21 mmol) in THF (10 mL) in an ice-water bath was added DBU (488.70 mg, 3.21 mmol) dropwise under N₂ atmosphere. The reaction mixture was allowed to warm to room and stirred for 5 h. Water (50 mL) and EA (50 mL) were added. The organic layer was separated. The aqueous layer was extracted with EA (2×50 mL). The combined organic layer was washed with brine (50 mL), dried over anhydrous Na₂SO₄ and evaporated to give crude product which was purified by prep-HPLC to give the product as a yellow oil (200 mg, yield: 40%).

### Step 5. (1-isobutyl-6-(pyridin-4-yl)-1H-indol-3-yl)methanamine

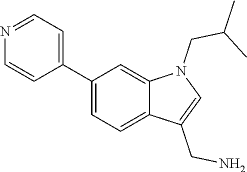 [SMILES: CC(C)Cn1cc(CN)c2ccc(-c3ccncc3)cc21]

A mixture of 3-(azidomethyl)-1-isobutyl-6-(pyridin-4-yl)-1H-indole (200 mg, 654.93 μmol) and triphenylphosphane (223.31 mg, 851.41 μmol) in THF (5 mL) was refluxed for 2 h. NH₃ in water (1 mL) was added. The reaction mixture was refluxed for another 2 h. The solvent was removed under reduced pressure. The residue was partitioned between EA (20 mL) and water (0.5 N HCl, 10 mL). The organic layer was separated. The aqueous layer was neutralized with 2 N aqueous K₂CO₃ until pH>8. The mixture was extracted with EA (2×20 mL). The combined organic layer was washed with brine (20 mL), dried over anhydrous Na₂SO₄ and evaporated to give the product as a yellow solid (150 mg, yield 81%).

### Step 6. dimethyl({[1-(2-methylpropyl)-6-(pyridin-4-yl)-1H-indol-3-yl]methyl}sulfamoyl)amine

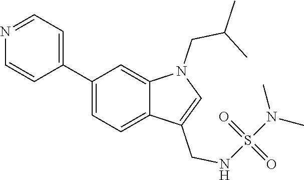 [SMILES: CC(C)Cn1cc(CNS(=O)(=O)N(C)C)c2ccc(-c3ccncc3)cc21]

To a solution of (1-isobutyl-6-(pyridin-4-yl)-1H-indol-3-yl)methanamine (65 mg, 0.23 mmol) and TEA (70.63 mg, 0.70 mmol) in THF (5.0 mL) in an ice-water bath, was added dimethylsulfamoyl chloride (33.41 mg, 0.23 μmol) dropwise. The reaction mixture was allowed to warm to room temperature and stirred overnight. The solvent was removed under reduced pressure. The residue was purified by prep-HPLC to give the product as a yellow solid (57 mg, yield: 63%). ¹H NMR (400 MHz, d₆-DMSO): δ 8.60-8.62 (m, 2H), 7.96 (d, J=0.8 Hz, 1H), 7.77-7.82 (m, 3H), 7.50-7.55 (m, 2H), 7.41 (s, 1H), 4.27 (d, J=6.0 Hz, 2H), 4.06 (d, J=7.2 Hz, 2H), 2.64 (s, 6H), 2.11-2.18 (m, 1H), 0.88 (d, J=6.4 Hz, 6H). LCMS (Method A) RT 1.996 min, calcd. for C₂₀H₂₆N₄O₂S 386.18 m/z, found 387.3 m/z [M+H]⁺.

### Example B-242: N-{[1-(2-methylpropyl)-6-(pyridin-4-yl)-1H-indol-3-yl]methyl}propane-2-sulfonamide

### Step 1. 6-bromo-1H-indole-3-carbaldehyde

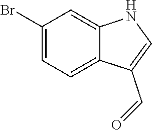 [SMILES: O=Cc1c[nH]c2cc(Br)ccc12]

To a solution of compound 6-bromoindole (5.0 g, 25.50 mmol) in DMF (20 mL) was added POCl₃ (5.87 g, 38.26 mmol). The mixture was stirred at 0° C. for 1 h. After the reaction was completed, the mixture was quenched with water and then extracted with EA (30 mL×3). The organic layer was separated, dried over Na₂SO₄ and concentrated in vacuo. The residue was purified by column chromatography to give the product as a yellow solid (4.9 g, 85% yield).

### Step 2. 6-bromo-1-isobutyl-1H-indole-3-carbaldehyde

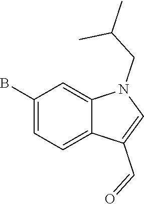

To a solution of 6-bromo-1H-indole-3-carbaldehyde (4.9 g, 21.87 mmol) in DMF (10 mL) was added KI (7.26 g, 43.74 mmol) and Cs₂CO₃ (14.25 g, 43.74 mmol). The mixture was stirred at 80° C. for 4 h. After the reaction was completed, the mixture was quenched with water and then extracted with EA (30 mL×3). The organic layer was separated, dried over Na₂SO₄ and concentrated in vacuo. The residue was purified by prep-HPLC to give the product as a yellow solid (6.0 g, 98% yield).

### Step 3. 1-isobutyl-6-(pyridin-4-yl)-1H-indole-3-carbaldehyde

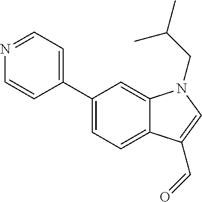 [SMILES: CC(C)Cn1cc(C=O)c2ccc(-c3ccncc3)cc21]

To a solution of 6-bromo-1-isobutyl-1H-indole-3-carbaldehyde (4.0 g, 14.34 mmol) in dioxane (10 mL) was added Pd(dppf)Cl₂ (1.05 g, 1.43 mmol), K₃PO₄·7H₂O (10.39 g, 43.02 mmol) and 4-(4,4,5,5-tetramethyl-1,3,2-dioxaborolan-2-yl)pyridine (3.52 g, 17.2 mmol). The mixture was stirred at 85° C. for 3 h. After the reaction was completed, the mixture was quenched with water and then extracted with EA (30 mL×3). The organic layer was separated, dried over Na₂SO₄ and concentrated in vacuo. The residue was purified by Prep-HPLC to give the product as a yellow solid (3.6 g, 90% yield).

### Step 4. (1-isobutyl-6-(pyridin-4-yl)-1H-indol-3-yl)methanamine

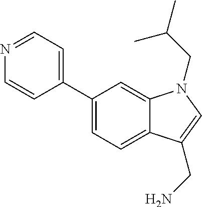 [SMILES: CC(C)Cn1cc(CN)c2ccc(-c3ccncc3)cc21]

To a solution of 1-isobutyl-6-(pyridin-4-yl)-1H-indole-3-carbaldehyde (1.0 g, 3.59 mmol) in MeOH (5 ml) was added NH₂OH·HCl (498.94 mg, 7.18 mmol). The mixture was stirred at room temperature for 6 hr. After the reaction was completed, the mixture was quenched with water and then extracted with EA (30 mL×3). Organic layer was separated, dried over Na₂SO₄ and concentrated in vacuo. Then mixture was dissolved in THF (10 mL) and added LiAlH₄ (163.49 mg, 4.31 mmol). The mixture was stirred at 80° C. for 1 hr. After the reaction was completed, the mixture was quenched with Na₂SO₄·10H₂O and then extracted with EA (30 mL×3). Organic layer was separated, dried over Na₂SO₄ and concentrated in vacuo. The residue was purified by prep-HPLC to give the product as a yellow solid (0.5 g, 49% yield).

### Step 5. N-((1-isobutyl-6-(pyridin-4-yl)-1H-indol-3-yl)methyl)propane-2-sulfonamide)

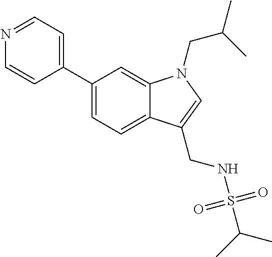 [SMILES: CC(C)Cn1cc(CNS(=O)(=O)C(C)C)c2ccc(-c3ccncc3)cc21]

To a solution of (1-isobutyl-6-(pyridin-4-yl)-1H-indol-3-yl)methanamine (50 mg, 17.90 μmol) and TEA (54.33 mg, 0.53 mmol) in DCM (5 mL) was added propane-2-sulfonyl chloride (75.22 mg, 0.53 mmol). The mixture was stirred at 0° C. for 1 hr. The solvent was removed under reduced pressure. The residue was purified by prep-HPLC to give the product as a white solid (6.6 mg, yield: 9%). ¹HNMR (400 MHz, DMSO-d₆) δ 8.59-8.61 (m, 2H), 7.96 (s, 1H), 7.79-7.82 (m, 3H), 7.49-7.51 (m, 1H), 7.43 (t, J=5.6 Hz, 1H), 7.40 (s, 1H), 4.31 (d, J=6.0 Hz, 2H), 4.06 (d, J=7.2 Hz, 2H), 2.98-3.01 (m, 1H), 2.12-2.17 (m, 1H), 1.15 (d, J=6.8 Hz, 6H), 0.86 (d, J=6.4 Hz, 6H). LCMS (Method A) RT 2.024 min, calcd. for C₂₁H₂₇N₃O₂S 385.5 m/z, found 386.4 m/z [M+H]⁺.

### Example B-243: N-{[1-(2-methylpropyl)-6-(pyridin-4-yl)-1H-indol-3-yl]methyl}cyclopentanesulfonamide

### Step 1. N-((1-isobutyl-6-(pyridin-4-yl)-1H-indol-3-yl)methyl)cyclopentanesulfonamide

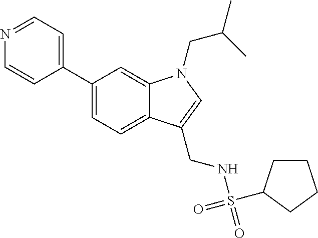 [SMILES: CC(C)Cn1cc(CNS(=O)(=O)C2CCCC2)c2ccc(-c3ccncc3)cc21]

To a solution of (1-isobutyl-6-(pyridin-4-yl)-1H-indol-3-yl)methanamine (50 mg, 17.90 μmol) and TEA (54.33 mg, 0.53 mmol) in DCM (5 mL) was added cyclopentanesulfonyl chloride (89.32 mg, 0.53 mmol). The mixture was stirred at room temperature for 12 hr. The solvent was removed under reduced pressure. The residue was purified by prep-HPLC to give the product as a yellow solid (21.79 mg, yield: 29%). ¹HNMR (400 MHz, DMSO-d₆) δ 8.57-8.59 (m, 2H), 7.94 (d, J=1.2 Hz, 1H), 7.77-7.79 (m, 3H), 7.47-7.50 (m, 1H), 7.41 (t, J=5.6 Hz, 1H), 7.38 (s, 1H), 4.29 (d, J=6.0 Hz, 2H), 4.04 (d, J=7.2 Hz, 2H), 3.35-3.37 (m, 1H), 2.10-2.14 (m, 1H), 1.70-1.84 (m, 4H), 1.56-1.59 (m, 2H), 1.41-1.46 (m, 2H), 0.85 (d, J=6.8 Hz, 6H). LCMS (Method A) RT 2.118 min, calcd. for C₂₃H₂₉N₃O₂S 411.5 m/z, found 412.4 m/z [M+H]⁺.

### Example B-244: N-{[1-(2-methylpropyl)-6-(pyridin-4-yl)-1H-indol-3-yl]methyl}pyrrolidine-1-sulfonamide

### Step 1. N-{[1-(2-methylpropyl)-6-(pyridin-4-yl)-1H-indol-3-yl]methyl}pyrrolidine-1-sulfonamide

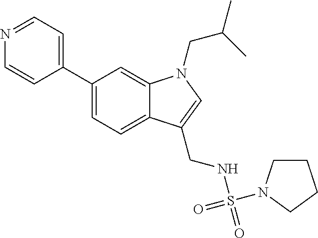 [SMILES: CC(C)Cn1cc(CNS(=O)(=O)N2CCCC2)c2ccc(-c3ccncc3)cc21]

A mixture of (1-isobutyl-6-(pyridin-4-yl)-1H-indol-3-yl)methanamine (300 mg, 1.07 mmol), pyrrolidine-1-sulfonyl chloride (273 mg, 1.61 mmol) and TEA (325 mg, 3.21 mmol) in THF (3 mL) was stirred at room temperature overnight. The reaction mixture was poured into water and extracted with EA (50 mL×3), dried and concentrated. The residue was purified by Prep-HPLC to give the product as a yellow solid (39 mg, yield: 9%). ¹HNMR (400 MHz, d₆-DMSO) δ 8.59 (d, J=6.4 Hz, 2H), 7.94 (s, 1H), 7.74-7.80 (m, 3H), 7.43-7.51 (m, 2H), 7.38 (s, 1H), 4.26 (d, J=5.6 Hz, 2H), 4.05 (d, J=7.6 Hz, 2H), 3.07-3.12 (m, 4H), 2.08-2.18 (m, 1H), 1.68-1.72 (m, 4H), 0.87 (d, J=6.8 Hz, 6H). LCMS (Method A) RT 2.088 min, calcd. for C₂₂H₂₈N₄O₂S 412.19 m/z, found 413.4 m/z [M+H]⁺.

### Example B-245: 2-methyl-N-{[1-(2-methylpropyl)-6-(pyridin-4-yl)-1H-indol-3-yl]methyl}propane-1-sulfonamide

### Step 1. N-((1-isobutyl-6-(pyridin-4-yl)-1H-indol-3-yl)methyl)-2-methylpropane-1-sulfonamide

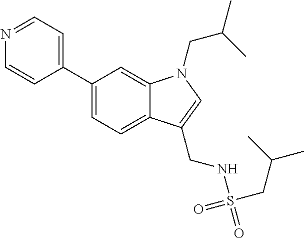 [SMILES: CC(C)Cn1cc(CNS(=O)(=O)CC(C)C)c2ccc(-c3ccncc3)cc21]

To a solution of of (1-isobutyl-6-(pyridin-4-yl)-1H-indol-3-yl)methanamine (50 mg, 17.90 μmol) and TEA (54.33 mg, 0.53 mmol) in DCM (5 mL) was added 2-methylpropane-1-sulfonyl chloride (83.01 mg, 0.53 mmol). The mixture was stirred at room temperature for 12 hr. The solvent was removed under reduced pressure. The residue was purified by prep-HPLC to give the product as a yellow solid (32.73 mg, yield: 45%). HNMR (400 MHz, DMSO-d₆) δ 8.57-8.59 (m, 2H), 7.94 (d, J=0.8 Hz, 1H), 7.77-7.79 (m, 3H), 7.48-7.50 (m, 1H), 7.44 (t, J=5.6 Hz, 1H), 7.39 (s, 1H), 4.28 (d, J=5.6 Hz, 2H), 4.04 (d, J=7.2 Hz, 2H), 2.70 (d, J=6.4 Hz, 2H), 2.08-2.18 (m, 1H), 1.93-2.00 (m, 1H), 0.80-0.86 (m, 12H). LCMS (Method A) RT 2.116 min, calcd. for C₂₂H₂₉N₃O₂S 399.5 m/z, found 400.4 m/z [M+H]⁺.

### Example B-246: dimethyl[methyl({[1-(2-methylpropyl)-6-(pyridin-4-yl)-1H-indol-3-yl]methyl})sulfamoyl]amine

### Step 1. tert-butyl (1-isobutyl-6-(pyridin-4-yl)-1H-indol-3-yl)methylcarbamate

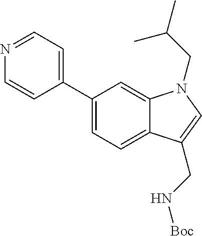 [SMILES: CC(C)Cn1cc(CNC(=O)OC(C)(C)C)c2ccc(-c3ccncc3)cc21]

A mixture of (1-isobutyl-6-(pyridin-4-yl)-1H-indol-3-yl)methanamine (450 mg, 1.61 mmol), (Boc)₂O (702 mg, 3.22 mmol) and TEA (326 mg, 3.22 mmol) in DCM (10 mL) was stirred at room temperature for 4 h. The reaction mixture was poured into water and extracted with EA (3×50 mL), dried and concentrated. The residue was purified by Prep-TLC (PE/EA=2:1) to give the product as a yellow solid (210 mg, 34% yield).

### Step 2. 1-(1-isobutyl-6-(pyridin-4-yl)-1H-indol-3-yl)-N-methylmethanamine

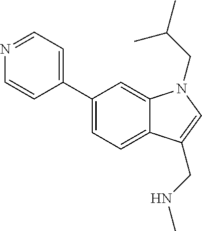 [SMILES: CNCc1cn(CC(C)C)c2cc(-c3ccncc3)ccc12]

To a solution of tert-butyl (1-isobutyl-6-(pyridin-4-yl)-1H-indol-3-yl)methylcarbamate (280 mg, 0.74 mmol) in THF (5 mL), was added LAH (115 mg, 2.95 mmol). The mixture was stirred at 75° C. for 3 hours and cooled to room temperature. Na₂SO₄·10H₂O was added. The mixture was stirred at room temperature for 0.5 hour. Then the reaction mixture was filtered and concentrated. The residue was purified by Prep-HPLC to give the product as a yellow oil (168 mg, yield: 77%).

### Step 3. dimethyl[methyl({[1-(2-methylpropyl)-6-(pyridin-4-yl)-1H-indol-3-yl]methyl})sulfamoyl]amine

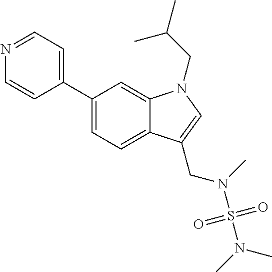 [SMILES: CC(C)Cn1cc(CN(C)S(=O)(=O)N(C)C)c2ccc(-c3ccncc3)cc21]

A mixture of 1-(1-isobutyl-6-(pyridin-4-yl)-1H-indol-3-yl)-N-methylmethanamine (150 mg, 0.51 mmol), dimethylsulfamoyl chloride (294 mg, 2.04 mmol) and TEA (259 mg, 2.56 mmol) in THF (3 mL) was stirred at room temperature overnight. The reaction mixture was poured into water and extracted with EA (3×50 mL). The combined organic layer was washed with brine, dried and concentrated to give crude product which was purified by. Prep-HPLC to give the product as a yellow solid (81 mg, yield: 39%). ¹HNMR (400 MHz, d₆-DMSO) δ 8.58-8.60 (m, 2H), 7.94-7.96 (m, 1H), 7.75-7.79 (m, 3H), 7.45-7.50 (m, 2H), 4.46 (s, 2H), 4.07 (d, J=7.2 Hz, 2H), 2.61 (s, 3H), 2.47-2.50 (m, 6H), 2.08-2.20 (m, 1H), 0.85 (d, J=6.8 Hz, 6H). LCMS (Method B) RT 2.965 min, calcd. for C₂₁H₂₈N₄O₂S 400.19 m/z, found 401.3 m/z [M+H]⁺.

### Example B-247: 6-bromo-1-(2-methylpropyl)-3-[2-(propane-2-sulfonyl)ethyl]-1H-indole

### Step 1. 1-(6-bromo-1-isobutyl-1H-indol-3-yl)-2-(isopropylthio)ethanone

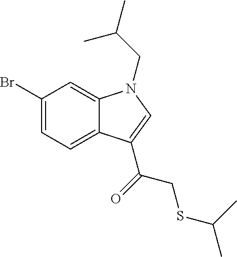 [SMILES: CC(C)Cn1cc(C(=O)CSC(C)C)c2ccc(Br)cc21]

A mixture of 1-(6-bromo-1-isobutyl-1H-indol-3-yl)-2-chloroethan-1-one (700 mg, 2.13 mmol), propane-2-thiol (350 mg, 4.60 mmol) and NaOH (280 mg, 7 mmol) in MeOH (15 mL) was stirred at 0° C. for 1 h. The reaction mixture was poured into water and extracted with EA (3×45 mL). The combined organic layer was washed with brine, dried and concentrated to give crude product which was purified by Prep-HPLC to give the product as a yellow oil (650 mg, yield: 83%).

### Step 2. 6-bromo-1-isobutyl-3-(2-(isopropylthio)ethyl)-1H-indole

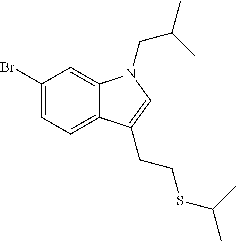 [SMILES: CC(C)Cn1cc(CCSC(C)C)c2ccc(Br)cc21]

A mixture of 1-(6-bromo-1-isobutyl-1H-indol-3-yl)-2-(isopropylthio)ethenone (700 mg, 1.90 mmol) in borane-THF (1 N in THF, 20 mL) was stirred at 65° C. overnight. Conc. HCl (12 N, 10 mL) was added. The mixture was stirred for 10 min and the solvent was removed under reduced pressure. The residue was purified by prep-HPLC to give the product as a yellow oil (600 mg, yield: 89%).

### Step 3. 6-bromo-1-isobutyl-3-(2-(isopropylsulfonyl)ethyl)-1H-indole

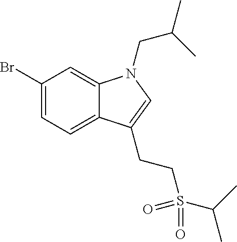 [SMILES: CC(C)Cn1cc(CCS(=O)(=O)C(C)C)c2ccc(Br)cc21]

To a stirred solution of 6-bromo-1-isobutyl-3-(2-(isopropylthio)ethyl)-1H-indole (500 mg, 1.41 mmol) in MeOH (15 mL), was added Oxone (900 mg, 2.93 mmol). The reaction mixture was stirred at room temperature for 30 minutes until the reaction was completed. The suspension was diluted with Na₂SO₃ (50 mL, aq.) and extracted with DCM (100 mL×2), concentrated. The crude product was purified by prep-HPLC to give the product as a yellow oil (290 mg, yield: 53%). ¹HNMR (400 MHz, d₆-DMSO) δ 7.71 (d, J=1.2 Hz, 1H), 7.49 (d, J=8.4 Hz, 1H), 7.30 (s, 1H), 7.13 (d, J=8.4 Hz, 1H), 3.89 (d, J=7.2 Hz, 2H), 3.34-3.39 (m, 2H), 3.20-3.32 (m, 1H), 3.04-3.12 (m, 2H), 2.00-2.07 (m, 1H), 1.22 (d, J=6.8 Hz, 6H), 0.81 (d, J=6.4 Hz, 6H). LCMS (Method B) RT 2.474 min, calcd. for C₁₇H₂₄BrNO₂S 385.07 m/z, found 386.2 m/z [M+H]⁺.

### Example B-248: 1-(2-methylpropyl)-3-[2-(propane-2-sulfonyl)ethyl]-6-(pyridin-4-yl)-1H-indole

### Step 1. 1-isobutyl-3-(2-(isopropylsulfonyl)ethyl)-6-(pyridin-4-yl)-1H-indole

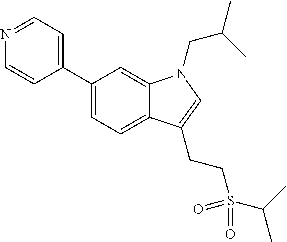 [SMILES: CC(C)Cn1cc(CCS(=O)(=O)C(C)C)c2ccc(-c3ccncc3)cc21]

To a suspension of 6-bromo-1-isobutyl-3-(2-(isopropylsulfonyl)ethyl)-1H-indole (130 mg, 336 μmol), 4-(4,4,5,5-tetramethyl-1,3,2-dioxaborolan-2-yl)pyridine (150 mg, 731.49 μmol) and K₃PO₄·7H₂O (220 mg, 1.04 mmol) in dioxane (3 mL), was added Pd(dppf)Cl₂ (30 mg, 41 μmol) under N₂ atmosphere. The reaction mixture was stirred at 90° C. for 1 hr. The resulting reaction was concentrated under reduced pressure. Water (25.0 mL) was added. The mixture was extracted with EA (2×25 mL). The combined organic layer was washed with brine (25 mL), dried over anhydrous Na₂SO₄, filtered, and concentrated under reduced pressure. The residue was purified by prep-HPLC to give the product as a yellow solid (22 mg, yield: 17%). ¹HNMR (400 MHz, d₆-DMSO) δ 8.60 (d, J=6.0 Hz, 2H), 7.95 (s, 1H), 7.79 (d, J=4.8 Hz, 2H), 7.68 (d, J=8.4 Hz, 1H), 7.48 (d, J=8.4 Hz, 1H), 7.41 (s, 1H), 4.03 (d, J=7.2 Hz, 2H), 3.39-3.45 (m, 2H), 3.24-3.31 (m, 1H), 3.12-3.18 (m, 2H), 2.11-2.17 (m, 1H), 1.25 (d, J=6.8 Hz, 6H), 0.87 (d, J=6.8 Hz, 6H). LCMS (Method B) RT 2.071 min, calcd. for C₂₂H₂₈N₂O₂S 384.19 m/z, found 385.3 m/z [M+H]⁺.

### Example B-249: 1-(2-methylpropyl)-6-phenyl-3-[2-(propane-2-sulfonyl)ethyl]-1H-indole

### Step 1. 1-isobutyl-3-(2-(isopropylsulfonyl)ethyl)-6-phenyl-1H-indole

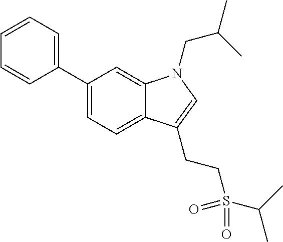 [SMILES: CC(C)Cn1cc(CCS(=O)(=O)C(C)C)c2ccc(-c3ccccc3)cc21]

To a suspension of 6-bromo-1-isobutyl-3-(2-(isopropylsulfonyl)ethyl)-1H-indole (130 mg, 336 μmol), phenylboronic acid (90 mg, 738.13 μmol) and K₃PO₄·7H₂O (220 mg, 1.04 mmol) in dioxane (3 mL), was added Pd(dppf)Cl₂ (30 mg, 41 μmol) under N₂ atmosphere. The reaction mixture was stirred at 90° C. for 1 hr. The resulting reaction was concentrated under reduced pressure. Water (25.0 mL) was added. The mixture was extracted with EA (2×25 mL). The combined organic layer was washed with brine (25 mL), dried over anhydrous Na₂SO₄, filtered, and concentrated under reduced pressure. The residue was purified by prep-HPLC to give the product as a white solid (25 mg, yield: 19%). HNMR (400 MHz, d₆-DMSO) δ 7.70-7.74 (m, 3H), 7.60-7.64 (m, 1H), 7.42 (t, J=7.6 Hz, 2H), 7.29-7.37 (m, 3H), 4.00 (d, J=7.2 Hz, 2H), 3.38-3.44 (m, 2H), 3.24-3.33 (m, 1H), 3.11-3.17 (m, 2H), 2.08-2.19 (m, 1H), 1.26 (d, J=6.8 Hz, 6H), 0.87 (d, J=6.4 Hz, 6H). LCMS (Method B) RT 2.557 min, calcd. for C₂₃H₂₉NO₂S 383.19 m/z, found 384.3 m/z [M+H]⁺.

### Example B-250: 6-cyclohexyl-1-(2-methylpropyl)-3-[2-(propane-2-sulfonyl)ethyl]-1H-indole

### Step 1. 6-cyclohexenyl-1-isobutyl-3-(2-(isopropylsulfonyl)ethyl)-1H-indole

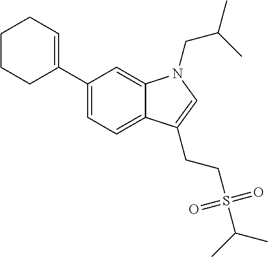 [SMILES: CC(C)Cn1cc(CCS(=O)(=O)C(C)C)c2ccc(C3=CCCCC3)cc21]

To a suspension of 6-bromo-1-isobutyl-3-(2-(isopropylsulfonyl)ethyl)-1H-indole (150 mg, 388 μmol), cyclohexenylboronic acid (73.33 mg, 582 μmol) and K₃PO₄·7H₂O (393.4 mg, 1.16 mmol) in dioxane (5 mL), was added Pd(dppf)Cl₂ (15 mg) under N₂ atmosphere. The reaction mixture was stirred at 90° C. for 1 hr. The resulting reaction was concentrated under reduced pressure. Water (25.0 mL) was added. The mixture was extracted with EA (2×25 mL). The combined organic layer was washed with brine (25 mL), dried over anhydrous Na₂SO₄, filtered, and concentrated under reduced pressure. The residue was purified by prep-HPLC to give the product as a white solid (50 mg, yield: 33%).

### Step 2. 6-cyclohexyl-1-(2-methylpropyl)-3-[2-(propane-2-sulfonyl)ethyl]-1H-indole

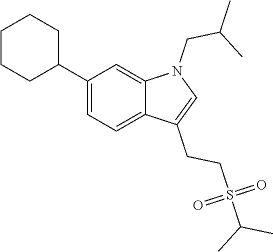 [SMILES: CC(C)Cn1cc(CCS(=O)(=O)C(C)C)c2ccc(C3CCCCC3)cc21]

To a mixture of 6-cyclohexenyl-1-isobutyl-3-(2-(isopropylsulfonyl)ethyl)-1H-indole (50 mg, 0.13 mmol) in EA (3 mL) and MeOH (1 mL), was added Pd/C (10 mg). The mixture was reacted under H₂ (1.0 atm.) at room temperature overnight. The solid was filtered off. The filtrate was evaporated to give the product as a white solid (33 mg, yield: 66%). T HNMR (400 MHz, d₆-DMSO) δ 7.42 (d, J=8.0 Hz, 1H), 7.24 (s, 1H), 7.19 (s, 1H), 6.91 (dd, J=8.0 Hz, 1.2 Hz 1H), 3.88 (d, J=7.2 Hz, 2H), 3.24-3.37 (m, 3H), 3.06-3.10 (m, 2H), 2.57 (t, J=11.6 Hz, 1H), 2.04-2.11 (m, 1H), 1.70-1.82 (m, 5H), 1.28-1.52 (m, 5H), 1.24 (d, J=6.4 Hz, 6H), 0.84 (d, J=6.8 Hz, 6H). LCMS (Method B) RT 2.754 min, calcd. for C₂₃H₃₅NO₂S 389.24 m/z, found 390.3 m/z [M+H]⁺.

### Example B-251: N-{2-[1-(2-methylpropyl)-6-(pyridin-4-yl)-1H-indol-3-yl]propyl}propane-2-sulfonamide

### Step 1. (E)-6-bromo-3-(2-nitrovinyl)-1H-indole

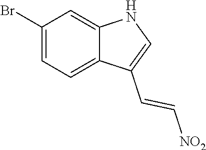 [SMILES: O=[N+]([O-])/C=C/c1c[nH]c2cc(Br)ccc12]

A mixture of 6-bromo-1H-indole-3-carbaldehyde (4 g, 17.85 mmol) and ammonium acetate (2.75 g, 35.71 mmol) in nitromethane (15 mL) was stirred at 70° C. for 1 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give the product as a yellow oil (3.5 g, yield: 73%).

### Step 2. 6-bromo-3-(1-nitropropan-2-yl)-1H-indole

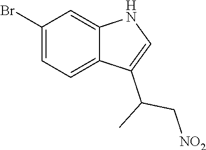 [SMILES: CC(C[N+](=O)[O-])c1c[nH]c2cc(Br)ccc12]

To a solution of (E)-6-bromo-3-(2-nitrovinyl)-1H-indole (3.3 g, 12.36 mmol) in anhydrous Et₂O (25 mL), was add a solution of methylmagnesium bromide (30.89 mmol, 10.3 mL) dropwise at −40° C. After stirred for 5 h, the reaction mixture was allowed to warm to room and quenched with a saturated aqueous solution of ammonium chloride. The resulting mixture was extracted with diethyl ether (3×50 ml). The combined organic layer was washed with brine (2×50 mL), dried over anhydrous Na₂SO₄ and evaporated to give the product as a yellow oil (3.3 g, yield: 94%).

### Step 3. 2-(6-bromo-1H-indol-3-yl)propan-1-amine

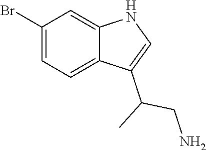

To a solution of 6-bromo-3-(1-nitropropan-2-yl)-1H-indole (3.3 g, 11.66 mmol) and conc. HCl (2 mL) in MeOH (4 mL) was added iron powder (3.25 g, 58.28 mmol). The mixture was refluxed for 2 hr. The solid was filtered and washed with MeOH. The combined organic layer was concentrated and purified by prep-HPLC to give the product as a yellow oil (2.52 g, yield: 85%).

### Step 4. tert-butyl 2-(6-bromo-1H-indol-3-yl)propylcarbamate

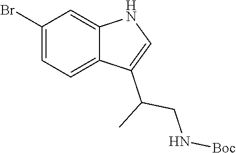 [SMILES: CC(CNC(=O)OC(C)(C)C)c1c[nH]c2cc(Br)ccc12]

To a solution of 2-(6-bromo-1H-indol-3-yl)propan-1-amine (2.52 g, 9.96 mmol) and DIPEA (3.86 g, 29.87 mmol) in DCM (30 mL), was added Boc₂O (2.82 g, 12.94 mmol) dropwise. The mixture was stirred for 1 h. The solvent was removed under reduced pressure. The residue was purified by silica gel column chromatography to give the product as a yellow oil (3.4 g, yield: 96%).

### Step 5. tert-butyl 2-(6-bromo-1-isobutyl-1H-indol-3-yl)propylcarbamate

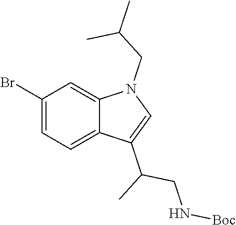 [SMILES: CC(C)Cn1cc(C(C)CNC(=O)OC(C)(C)C)c2ccc(Br)cc21]

To a solution of tert-butyl 2-(6-bromo-1H-indol-3-yl)propylcarbamate (3.4 g, 9.62 mmol) and 1-bromo-2-methyl-propane (2.64 g, 19.25 mmol) in DMF (30 mL), were added Cs₂CO₃ (6.27 g, 19.25 mmol) and KI (0.3 g). The mixture was stirred at 80° C. for 3 d, and then cooled to room temperature. Water (100 mL) and EA (100 mL) was added. The organic layer was separated. The aqueous layer was extracted with EA (2×100 mL). The combined organic layer was washed with brine (4×50 mL), dried over anhydrous Na₂SO₄ and evaporated to give crude product which was purified by silica gel column chromatography to give the product as a yellow oil (2.7 g, yield: 68%).

### Step 6. tert-butyl 2-(1-isobutyl-6-(pyridin-4-yl)-1H-indol-3-yl)propylcarbamate

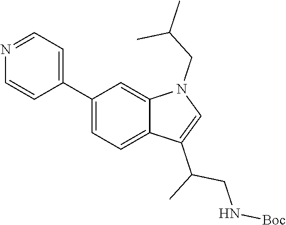 [SMILES: CC(C)Cn1cc(C(C)CNC(=O)OC(C)(C)C)c2ccc(-c3ccncc3)cc21]

To a suspension of tert-butyl 2-(6-bromo-1-isobutyl-1H-indol-3-yl)propylcarbamate (900 mg, 2.20 mmol), 4-(4,4,5,5-tetramethyl-1,3,2-dioxaborolan-2-yl)pyridine (450.84 mg, 2.20 mmol) and K₃PO₄·7H₂O (2.23 g, 6.60 mmol) in dioxane (10 mL), was added Pd(dppf)Cl₂ (160.87 mg, 219.86 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (750 mg, yield: 83%).

### Step 7. 2-(1-isobutyl-6-(pyridin-4-yl)-1H-indol-3-yl)propan-1-amine

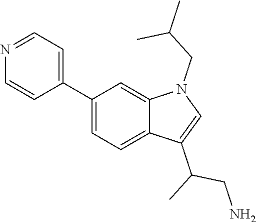 [SMILES: CC(C)Cn1cc(C(C)CN)c2ccc(-c3ccncc3)cc21]

A mixture of tert-butyl 2-(1-isobutyl-6-(pyridin-4-yl)-1H-indol-3-yl)propylcarbamate (750 mg, 1.84 mmol) in DCM (8 mL) and TFA (4 mL) was stirred at room temperature for 1 h. The solvent was removed under reduced pressure. The residue was dissolved in EA (50 mL), neutralized with 1 N K₂CO₃ until pH>8, washed with brine, dried over anhydrous Na₂SO₄ and evaporated to give the product as a yellow oil (460 mg, yield: 81%).

### Step 8 N-{2-[1-(2-methylpropyl)-6-(pyridin-4-yl)-1H-indol-3-yl]propyl}propane-2-sulfonamide

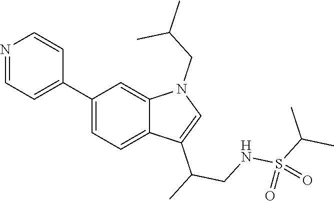 [SMILES: CC(C)Cn1cc(C(C)CNS(=O)(=O)C(C)C)c2ccc(-c3ccncc3)cc21]

To a mixture of 2-(1-isobutyl-6-(pyridin-4-yl)-1H-indol-3-yl)propan-1-amine (100 mg, 325.28 μmol) and DIPEA (98.75 mg, 975.84 μmol) in DCM (5 mL) in an ice water bath, was added propane-2-sulfonyl chloride (46.39 mg, 325.28 μmol). The mixture was allowed to warm to room temperature and stirred overnight. The solvent was removed under reduced pressure. The residue was purified by prep-HPLC to give the product as a yellow solid (14.25 mg, yield: 10%). ¹H NMR (400 MHz, d₆-DMSO): δ 8.59-8.61 (m, 2H), 8.47 (s, 1H), 7.92 (d, J=1.2 Hz, 1H), 7.78-7.79 (m, 2H), 7.68 (d, J=8.4 Hz, 1H), 7.45-7.47 (m, 1H), 7.29 (s, 1H), 7.15 (t, J=5.6 Hz, 1H), 4.03 (d, J=7.2 Hz, 2H), 3.28-3.34 (m, 1H), 3.07-3.18 (m, 2H), 2.95-3.02 (m, 1H), 2.10-2.18 (m, 1H), 1.33 (d, J=7.2 Hz, 3H), 1.16-1.19 (m, 6H), 0.86-0.89 (m, 6H). LCMS (Method A) RT 2.172 min, calcd. for C₂₃H₃₁N₃O₂S 413.21 m/z, found 414.3 m/z [M+H]⁺.

### Example B-252: dimethyl({2-[1-(2-methylpropyl)-6-(pyridin-4-yl)-1H-indol-3-yl]propyl}sulfamoyl)amine

### Step 1. dimethyl({2-[1-(2-methylpropyl)-6-(pyridin-4-yl)-1H-indol-3-yl]propyl}sulfamoyl)amine

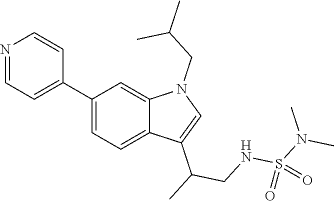 [SMILES: CC(C)Cn1cc(C(C)CNS(=O)(=O)N(C)C)c2ccc(-c3ccncc3)cc21]

To a mixture of 2-(1-isobutyl-6-(pyridin-4-yl)-1H-indol-3-yl)propan-1-amine (100 mg, 325.28 μmol) and DIPEA (98.75 mg, 975.84 μmol) in CH₃CN (5 mL) in an ice-water bath, was added dimethylsulfamoyl chloride (70.06 mg, 487.91 μmol). The mixture was allowed to warm to room temperature and stirred overnight. The solvent was removed under reduced pressure. The residue was purified by prep-HPLC to give the product as a yellow solid (42 mg, yield: 31%). ¹H NMR (400 MHz, d₆-DMSO): δ 8.57-8.59 (m, 2H), 7.90 (d, J=1.2 Hz, 1H), 7.76-7.77 (m, 2H), 7.65 (d, J=8.4 Hz, 1H), 7.43-7.46 (m, 1H), 7.29 (s, 1H), 7.26 (s, 1H), 4.01 (d, J=7.2 Hz, 2H), 3.22-3.27 (m, 1H), 3.12-3.17 (m, 1H), 2.89-2.95 (m, 1H), 2.60 (s, 6H), 2.08-2.18 (m, 1H), 1.32 (d, J=6.8 Hz, 3H), 0.85 (d, J=6.0 Hz, 6H). LCMS (Method A) RT 2.167 min, calcd. for C₂₂H₃₀N₄O₂S 414.21 m/z, found 415.3 m/z [M+H]⁺.

### Example B-253: N-{2-[1-(2-methylpropyl)-6-(pyridin-4-yl)-1H-indol-3-yl]propyl}cyclopropanesulfonamide

### Step 1. N-{2-[1-(2-methylpropyl)-6-(pyridin-4-yl)-1H-indol-3-yl]propyl}cyclopropanesulfonamide

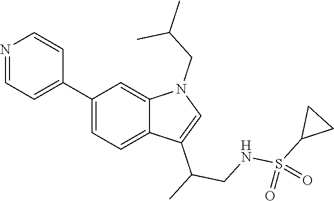 [SMILES: CC(C)Cn1cc(C(C)CNS(=O)(=O)C2CC2)c2ccc(-c3ccncc3)cc21]

To a mixture of 2-(1-isobutyl-6-(pyridin-4-yl)-1H-indol-3-yl)propan-1-amine (100 mg, 325.28 μmol) and DIPEA (98.75 mg, 975.84 μmol) in DCM (5 mL) in an ice water bath, was added cyclopropanesulfonyl chloride (68.59 mg, 487.91 μmol). The mixture was allowed to warm to room temperature and stirred for 4 h. The solvent was removed under reduced pressure. The residue was purified by prep-HPLC to give the product as a yellow solid (52.68 mg, yield: 39%). ¹H NMR (400 MHz, d₆-DMSO): δ 8.57-8.59 (m, 2H), 7.90 (d, J=1.2 Hz, 1H), 7.76-7.77 (m, 2H), 7.66 (d, J=8.4 Hz, 1H), 7.43-7.46 (m, 1H), 7.27 (s, 1H), 7.18 (s, 1H), 4.01 (d, J=7.6 Hz, 2H), 3.31-3.35 (m, 1H), 3.12-3.18 (m, 1H), 2.97-3.02 (m, 1H), 2.46-2.51 (m, 1H), 2.10-2.16 (m, 1H), 1.33 (d, J=6.4 Hz, 3H), 0.84-0.87 (m, 10H). LCMS (Method A) RT 2.141 min, calcd. for C₂₃H₂₉N₃O₂S 411.20 m/z, found 412.4 m/z [M+H]⁺.

### Example B-254: N-{2-[1-(2-methylpropyl)-6-phenyl-1H-indol-3-yl]propyl}propane-2-sulfonamide

### Step 1. tert-butyl 2-(1-isobutyl-6-phenyl-1H-indol-3-yl)propylcarbamate

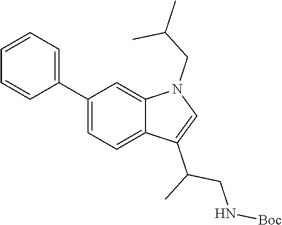 [SMILES: CC(C)Cn1cc(C(C)CNC(=O)OC(C)(C)C)c2ccc(-c3ccccc3)cc21]

To a suspension of tert-butyl (2-(6-bromo-1-isobutyl-1H-indol-3-yl)propyl)carbamate (1.00 g, 2.44 mmol), 4,4,5,5-tetramethyl-2-phenyl-1,3,2-dioxaborolane (648 mg, 3.18 mmol) and K₃PO₄·7H₂O (2.48 g, 7.33 mmol) in dioxane (10 mL), was added Pd(dppf)Cl₂ (178 mg, 244 μmol) under N₂ atmosphere. The reaction mixture was reacted at 85° C. for 3 h. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (790 mg, yield: 79%).

### Step 2. 2-(1-isobutyl-6-phenyl-1H-indol-3-yl)propan-1-amine

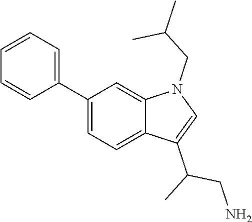 [SMILES: CC(C)Cn1cc(C(C)CN)c2ccc(-c3ccccc3)cc21]

A mixture of tert-butyl 2-(1-isobutyl-6-phenyl-1H-indol-3-yl)propylcarbamate (790 mg, 1.94 mmol) in DCM (8 mL) and TFA (4 mL) was stirred at room temperature for 1 h. The solvent was removed under reduced pressure. The residue was dissolved in EA (50 mL), neutralized with 1 N K₂CO₃ until pH>8, washed with brine, dried over anhydrous Na₂SO₄ and evaporated to give the product as a yellow oil (510 mg, yield: 85%).

### Step 3. N-{2-[1-(2-methylpropyl)-6-phenyl-1H-indol-3-yl]propyl}propane-2-sulfonamide

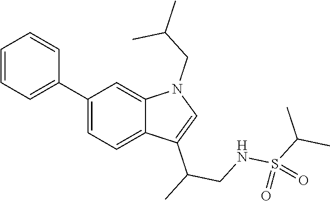 [SMILES: CC(C)Cn1cc(C(C)CNS(=O)(=O)C(C)C)c2ccc(-c3ccccc3)cc21]

To a mixture of 2-(1-isobutyl-6-phenyl-1H-indol-3-yl)propan-1-amine (100 mg, 325.28 μmol) and DIPEA (42.18 mg, 326.32 μmol) in DCM (5 mL) in an ice-water bath, was added propane-2-sulfonyl chloride (46.54 mg, 326.32 μmol). The mixture was allowed to warm to room temperature and stirred overnight. The solvent was removed under reduced pressure. The residue was purified by prep-HPLC to give the product as a white solid (13.23 mg, yield: 9%). ¹H NMR (400 MHz, d₆-DMSO): δ 7.69-7.72 (m, 3H), 7.63 (d, J=8.4 Hz, 1H), 7.45 (t, J=7.6 Hz, 2H), 7.30-7.33 (m, 2H), 7.21 (s, 1H), 7.13 (s, 1H), 3.99 (d, J=7.2 Hz, 2H), 3.30-3.32 (m, 1H), 3.08-3.15 (m, 2H), 2.95-3.01 (m, 1H), 2.07-2.19 (m, 1H), 1.34 (d, J=6.8 Hz, 3H), 1.17-1.19 (m, 6H), 0.86-0.88 (m, 6H). LCMS (Method A) RT 2.545 min, calcd. for C₂₄H₃₂N₂O₂S 412.22 m/z, found 413.4 m/z [M+H]⁺.

### Example B-255: dimethyl({2-[1-(2-methylpropyl)-6-phenyl-1H-indol-3-yl]propyl}sulfamoyl)amine

### Step 1. dimethyl({2-[1-(2-methylpropyl)-6-phenyl-1H-indol-3-yl]propyl}sulfamoyl)amine

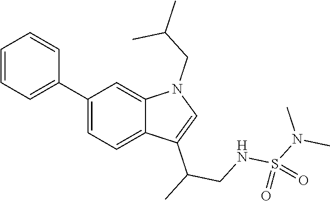 [SMILES: CC(C)Cn1cc(C(C)CNS(=O)(=O)N(C)C)c2ccc(-c3ccccc3)cc21]

To a mixture of 2-(1-isobutyl-6-phenyl-1H-indol-3-yl)propan-1-amine (100 mg, 326.32 μmol) and DIPEA (42.18 mg, 326.32 μmol) in DMF (5 mL) in an ice-water bath, was added dimethylsulfamoyl chloride (46.86 mg, 326.32 μmol). The mixture was allowed to warm to room temperature and stirred for 1 h. The solvent was removed under reduced pressure. The residue was purified by prep-HPLC to give the product as a white solid (35 mg, yield: 25%). ¹H NMR (400 MHz, d₆-DMSO): δ 7.70-7.72 (m, 3H), 7.62 (d, J=8.4 Hz, 1H), 7.45 (t, J=7.6 Hz, 2H), 7.30-7.33 (m, 3H), 7.22 (s, 1H), 3.99 (d, J=7.6 Hz, 2H), 3.25-3.30 (m, 1H), 3.13-3.16 (m, 1H), 2.90-2.96 (m, 1H), 2.63 (s, 6H), 2.10-2.17 (m, 1H), 1.34 (d, J=6.8 Hz, 3H), 0.86 (d, J=6.0 Hz, 6H). LCMS (Method A) RT 2.539 min, calcd. for C₂₃H₃₁N₃O₂S 413.21 m/z, found 414.4 m/z [M+H]+.

### Example B-256: N-{2-[1-(2-methylpropyl)-6-phenyl-1H-indol-3-yl]propyl}cyclopropanesulfonamide

### Step 1. N-(2-(1-isobutyl-6-phenyl-1H-indol-3-yl)propyl)cyclopropanesulfonamide

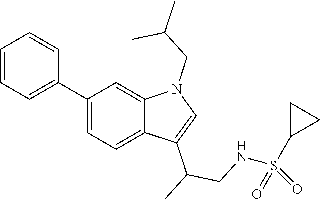 [SMILES: CC(C)Cn1cc(C(C)CNS(=O)(=O)C2CC2)c2ccc(-c3ccccc3)cc21]

To a mixture of 2-(1-isobutyl-6-phenyl-1H-indol-3-yl)propan-1-amine (100 mg, 326.32 μmol) and DIPEA (126.53 mg, 978.97 μmol) in DMF (5 mL) in an ice-water bath, was added cyclopropanesulfonyl chloride (45.88 mg, 326.32 μmol). The mixture was allowed to warm to room temperature and stirred for 5 h. The solvent was removed under reduced pressure. The residue was purified by prep-HPLC to give the product as a white solid (47 mg, yield: 35%). ¹H NMR (400 MHz, d₆-DMSO): δ 7.70-7.72 (m, 3H), 7.63 (d, J=8.0 Hz, 1H), 7.45 (t, J=7.6 Hz, 2H), 7.30-7.33 (m, 2H), 7.21 (s, 1H), 7.19 (s, 1H), 3.99 (d, J=7.2 Hz, 2H), 3.30-3.38 (m, 1H), 3.13-3.18 (m, 1H), 2.98-3.03 (m, 1H), 2.49-2.55 (m, 1H), 2.11-2.17 (m, 1H), 1.35 (d, J=6.8 Hz, 3H), 0.86-0.89 (m, 10H). LCMS (Method A) RT 2.504 min, calcd. for C₂₄H₃₀N₂O₂S 410.20 m/z, found 411.3 m/z [M+H]⁺.

### Example B-257: N-[rel-(1R,2R)-2-[6-bromo-1-(2-methylpropyl)-1H-indol-3-yl]cyclopropyl]propane-2-sulfonamide

### Step 1. 6-bromo-1-isobutyl-3-vinyl-1H-indole

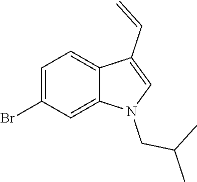 [SMILES: C=Cc1cn(CC(C)C)c2cc(Br)ccc12]

To a suspension of 6-bromo-1-isobutyl-1H-indole-3-carbaldehyde (6.0 g, 21.42 mmol) and iodo-methyl-triphenyl-phosphane (9.52 g, 23.56 mmol) in THF (120 mL), was added potassium tert-butoxide (2.52 g, 22.49 mmol) in portions under ice-water. The mixture was stirred for 2 h at 0° C. Water (200 mL) and EA (200 mL) was added. The organic layer was separated. The aqueous layer was extracted with EA (2×100 mL). The combined organic layers was washed with brine (4×50 mL), dried over anhydrous Na₂SO₄ and evaporated to give crude product which was purified by silica gel column chromatography to give the product as a yellow oil (4 g, yield: 67%).

### Step 2. rel-(1R,2R)-ethyl 2-(6-bromo-1-isobutyl-1H-indol-3-yl)cyclopropanecarboxylate

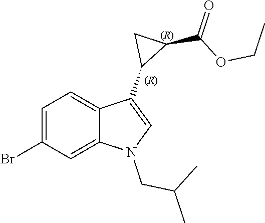

To a solution of 6-bromo-1-isobutyl-3-vinyl-1H-indole (4 g, 14.38 mmol) and diacetoxyrhodium (317.76 mg, 718.94 μmol) in DCM (100 mL), was added ethyl 2-diazoacetate (3.28 g, 28.76 mmol) in DCM (50 mL) dropwise. The mixture was allowed to warm to room temperature, and then stirred for another 2 h. The solvent was removed under reduced pressure. The residue was purified by silica gel column chromatography and prep-HPLC to give the product as a yellow oil (2.2 g, yield: 42%).

### Step 3. rel-(1R,2R)-2-(6-bromo-1-isobutyl-1H-indol-3-yl)cyclopropanecarboxylic acid

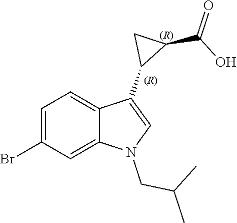

To a solution of rel-(1R,2R)-ethyl 2-(6-bromo-1-isobutyl-1H-indol-3-yl)cyclopropanecarboxylate (2.2 g, 6.04 mmol) in MeOH (20 mL) was added lithium hydroxide hydrate (1.27 g, 30.20 mmol) in portions. The mixture was stirred at room temperature for 2 h. The solvent was removed under reduced pressure. The residue was dissolved in water (50 mL). 1N aqueous HCl was added until pH>5. The mixture was extracted with EA (3×50 mL). The combined organic layer was washed with brine (50 mL×4), dried over anhydrous Na₂SO₄ and evaporated to give the product as a yellow oil (1.8 g, yield: 88%).

### Step 4. rel-(1R,2R)-2-(6-bromo-1-isobutyl-1H-indol-3-yl)cyclopropanamine

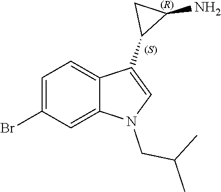

To a solution of rel-(1R,2R)-2-(6-bromo-1-isobutyl-1H-indol-3-yl)cyclopropanecarboxylic acid (1.8 g, 5.35 mmol) and TEA (541.73 mg, 5.35 mmol) in CH₃CN (80 mL), was added DPPA (1.47 g, 5.35 mmol). The resulting mixture was heated at 50° C. for 2 h. After cooling to room temperature, aqueous 1 N HCl (90 mL) was added and then the reaction mixture was refluxed overnight. The solvent was removed under reduced pressure. The residue was neutralized with aqueous 1 N NaOH until pH>14. The aqueous layer was extracted with DCM (3×50 mL). The combined organic layer was dried over Na₂SO₄, filtered and evaporated to give crude product which was purified by prep-HPLC to give the product as a yellow oil (1.0 g, yield: 60%).

### Step 5. N-[rel-(1R,2R)-2-[6-bromo-1-(2-methylpropyl)-1H-indol-3-yl]cyclopropyl]propane-2-sulfonamide

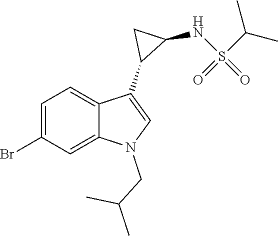 [SMILES: CC(C)Cn1cc([C@@H]2C[C@H]2NS(=O)(=O)C(C)C)c2ccc(Br)cc21]

To a mixture of rel-(1R,2R)-2-(6-bromo-1-isobutyl-1H-indol-3-yl)cyclopropanamine (600 mg, 1.95 mmol)) and TEA (592.86 mg, 5.86 mmol) in DCM (20 mL) in an ice-water bath, was added propane-2-sulfonyl chloride (557.00 mg, 3.91 mmol) dropwise. The mixture was allowed to warm to room temperature and stirred overnight. The solvent was removed under reduced pressure. The residue was purified by prep-HPLC to give the product as a yellow solid (280 mg, yield: 34%). ¹H NMR (400 MHz, d₆-DMSO): δ 7.65-7.69 (m, 3H), 7.11 (dd, J=8.4 Hz, 2.0 Hz, 1H), 7.06 (s, 1H), 3.80-3.90 (m, 2H), 3.21-3.28 (m, 1H), 2.38-2.41 (m, 1H), 2.09-2.14 (m, 1H), 1.98-2.05 (m, 1H), 1.20-1.24 (m, 6H), 1.08-1.12 (m, 2H), 0.79 (dd, J=6.8 Hz, 1.2 Hz, 6H). LCMS (Method A) RT 2.432 min, calcd. for C₁₈H₂₅BrN₂O₂S 412.08 m/z, found 413.3 m/z [M+H]⁺.

### Example B-258: N-[(rel-1R,2R)-2-[1-(2-methylpropyl)-6-(pyridin-4-yl)-1H-indol-3-yl]cyclopropyl]propane-2-sulfonamide

### Step 1. N-[(rel-1R,2R)-2-[1-(2-methylpropyl)-6-(pyridin-4-yl)-1H-indol-3-yl]cyclopropyl]propane-2-sulfonamide

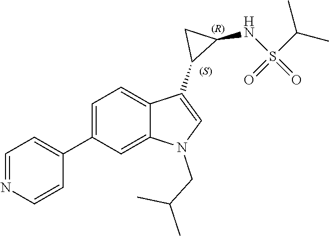

To a suspension of N-[rel-(1R,2R)-2-[6-bromo-1-(2-methylpropyl)-1H-indol-3-yl]cyclopropyl]propane-2-sulfonamide (100 mg, 241.91 μmol), 4-(4,4,5,5-tetramethyl-1,3,2-dioxaborolan-2-yl)pyridine (49.61 mg, 241.91 μmol) and K₃PO₄·7H₂O (245.30 mg, 725.74 μmol) in dioxane (5 mL), was added Pd(dppf)Cl₂ (17.70 mg, 24.19 μmol) under N₂ atmosphere. The reaction mixture was reacted at 85° C. for 3 h and cooled to room temperature. The resulting reaction mixture was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (18.9 mg, yield: 18%). ¹H NMR (400 MHz, d₆-DMSO): δ 8.57-8.58 (m, 2H), 7.89 (d, J=0.8 Hz, 1H), 7.84 (d, J=8.0 Hz, 1H), 7.75-7.78 (m, 2H), 7.62 (br, 1H), 7.46 (dd, J=8.4 Hz, 1.6 Hz, 1H), 7.15 (s, 1H), 3.92-4.02 (m, 2H), 3.24-3.31 (m, 1H), 2.42-2.46 (m, 1H), 2.05-2.19 (m, 2H), 1.22-1.26 (m, 6H), 1.12-1.15 (m, 2H), 0.82 (dd, J=6.8 Hz, 1.2 Hz, 6H). LCMS (Method A) RT 2.121 min, calcd. for C₂₃H₂₉N₃O₂S 411.20 m/z, found 412.4 m/z [M+H]⁺.

### Example B-259: N-[rel-(1R,2R)-2-[1-(2-methylpropyl)-6-phenyl-1H-indol-3-yl]cyclopropyl]propane-2-sulfonamide

### Step 1. N-[rel-(1R,2R)-2-[1-(2-methylpropyl)-6-phenyl-1H-indol-3-yl]cyclopropyl]propane-2-sulfonamide

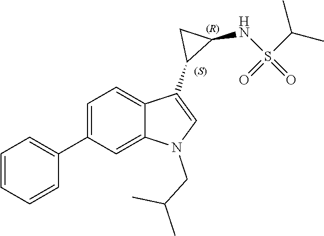

To a suspension of N-[rel-(1R,2R)-2-[6-bromo-1-(2-methylpropyl)-1H-indol-3-yl]cyclopropyl]propane-2-sulfonamide (100 mg, 241.91 μmol), phenylboronic acid (35.40 mg, 290.30 μmol) and K₃PO₄·7H₂O (245.30 mg, 725.74 μmol) in dioxane (5 mL), was added Pd(dppf)Cl₂ (17.70 mg, 24.19 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (18.63 mg, yield: 18%). ¹H NMR (400 MHz, d₆-DMSO): δ 7.78 (d, J=8.0 Hz, 1H), 7.59-7.71 (m, 4H), 7.43 (t, J=7.6 Hz, 2H), 7.28-7.32 (m, 2H), 7.07 (s, 1H), 3.88-3.99 (m, 2H), 3.24-3.31 (m, 1H), 2.42-2.45 (m, 1H), 2.08-2.18 (m, 2H), 1.22-1.26 (m, 6H), 1.11-1.14 (m, 2H), 0.82 (d, J=6.0 Hz, 6H). LCMS (Method A) RT 2.491 min, calcd. for C₂₄H₃₀N₂O₂S 410.20 m/z, found 411.4 m/z [M+H]⁺

### Example B-260: N-{2-[5-fluoro-1-(2-methylpropyl)-6-(pyridin-4-yl)-1H-indol-3-yl]ethyl}propane-2-sulfonamide

### Step 1. (E)-6-bromo-5-fluoro-1-isobutyl-3-(2-nitrovinyl)-1H-indole

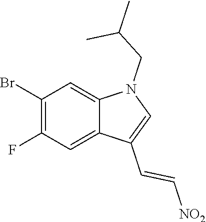 [SMILES: CC(C)Cn1cc(/C=C/[N+](=O)[O-])c2cc(F)c(Br)cc21]

To a solution of 6-bromo-5-fluoro-1-isobutyl-1H-indole-3-carbaldehyde (2.5 g, 8.39 mmol) in nitromethane (9 mL), was added AcONH₄ (2.5 g, 32.43 mmol). The mixture was stirred at 70° C. for 1 hr. The resulting reaction was concentrated under reduced pressure, and water (30 mL) was added. The mixture was extracted with EA (2×30 mL). The combined organic layer was washed with brine (2×40 mL), dried over Na₂SO₄, filtered, and concentrated to give the product as a yellow oil (2.5 g, yield: 87%).

### Step 2. 6-bromo-5-fluoro-1-isobutyl-3-(2-nitroethyl)-1H-indole

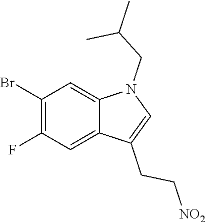 [SMILES: CC(C)Cn1cc(CC[N+](=O)[O-])c2cc(F)c(Br)cc21]

To a solution of (E)-6-bromo-5-fluoro-1-isobutyl-3-(2-nitrovinyl)-1H-indole (2.5 g, 7.33 mmol) in MeOH (12 mL), was added NaBH₄ (1.36 g, 36.04 mmol) in portions at 0° C. The mixture was stirred for 1 h at that temperature, and poured into ice-water. The solvent was removed under reduced pressure. The residue was extracted with EA (2×50 mL). The combined organic layer was washed with brine (2×50 mL), dried and evaporated to give crude product which was used to the next step directly.

### Step 3. 2-(6-bromo-5-fluoro-1-isobutyl-1H-indol-3-yl)ethanamine

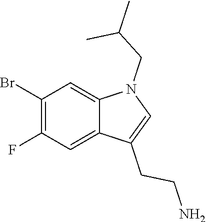 [SMILES: CC(C)Cn1cc(CCN)c2cc(F)c(Br)cc21]

To a solution of 6-bromo-5-fluoro-1-isobutyl-3-(2-nitroethyl)-1H-indole (2.3 g, 6.70 mmol) and HCl (12 N, 3 mL) in MeOH (10 mL), was added Fe (1.84 g, 32.95 mmol). The mixture was refluxed for 2 hr. The solid was filtered off and washed with MeOH. The combined organic layer was concentrated and purified by prep-HPLC to give the product as a yellow solid (1.1 g, yield: 52%).

### Step 4. N-(2-(6-bromo-5-fluoro-1-isobutyl-1H-indol-3-yl)ethyl)propane-2-sulfonamide

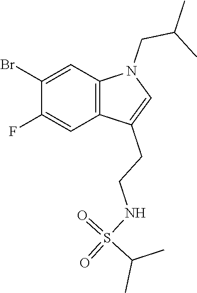 [SMILES: CC(C)Cn1cc(CCNS(=O)(=O)C(C)C)c2cc(F)c(Br)cc21]

To a solution of 2-(6-bromo-5-fluoro-1-isobutyl-1H-indol-3-yl)ethanamine (1.1 g, 3.51 mmol) and DIEA (2.04 g, 15.79 mmol) in DCM (3 mL), was added propane-2-sulfonyl chloride (1.1 g, 7.71 mmol) dropwise at −20° C. The mixture was allowed to warm to room temperature and stirred for 1 h. The solvent was removed under reduced pressure. The residue was purified by prep-HPLC to give the product as a white solid (500 mg, yield: 33%).

### Step 5. N-{2-[5-fluoro-1-(2-methylpropyl)-6-(pyridin-4-yl)-1H-indol-3-yl]ethyl}propane-2-sulfonamide

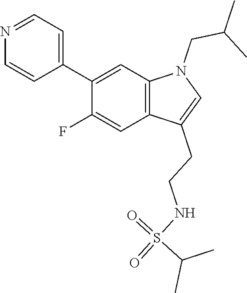 [SMILES: CC(C)Cn1cc(CCNS(=O)(=O)C(C)C)c2cc(F)c(-c3ccncc3)cc21]

To a suspension of N-(2-(6-bromo-5-fluoro-1-isobutyl-1H-indol-3-yl)ethyl)propane-2-sulfonamide (100 mg, 238.46 μmol), 4-(4,4,5,5-tetramethyl-1,3,2-dioxaborolan-2-yl)pyridine (100 mg, 487.66 μmol) and K₃PO₄·7H₂O (300 mg, 1.41 mmol) in dioxane (3 mL), was added Pd(dppf)Cl₂ (100 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (25 mg, yield: 25%). ¹H NMR (400 MHz, d₆-DMSO): δ 8.62 (d, J=4.4 Hz, 2H), 7.70 (d, J=6.8 Hz, 1H), 7.61 (d, J=4.4 Hz, 2H), 7.43 (d, J=12.0 Hz, 1H), 7.37 (s, 1H), 7.08 (s, 1H), 3.98 (d, J=7.2 Hz, 2H), 3.09-3.22 (m, 3H), 2.84 (t, J=7.6 Hz, 2H), 2.05-2.15 (s, 1H), 1.17 (d, J=6.8 Hz, 6H), 0.83 (d, J=6.8 Hz, 6H). LCMS (Method A) RT 2.224 min, calcd. for C₂₂H₂₈FN₃O₂S 417.19 m/z, found 418.3 m/z [M+H]⁺.

### Example B-261: N-{2-[5-fluoro-1-(2-methylpropyl)-6-phenyl-1H-indol-3-yl]ethyl}propane-2-sulfonamide

### Step 1. N-{2-[5-fluoro-1-(2-methylpropyl)-6-phenyl-1H-indol-3-yl]ethyl}propane-2-sulfonamide

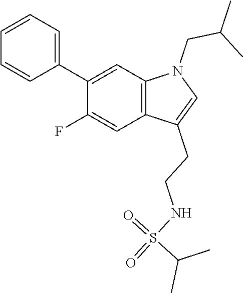 [SMILES: CC(C)Cn1cc(CCNS(=O)(=O)C(C)C)c2cc(F)c(-c3ccccc3)cc21]

To a suspension of N-(2-(6-bromo-5-fluoro-1-isobutyl-1H-indol-3-yl)ethyl)propane-2-sulfonamide (100 mg, 238.46 μmol), phenylboronic acid (100 mg, 820.14 μmol) and K₃PO₄·7H₂O (300 mg, 1.41 mmol) in dioxane (3 mL), was added Pd(dppf)Cl₂ (100 mg) under N₂ atmosphere. The reaction mixture was reacted at 85° C. for 30 min and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (28 mg, yield: 28%). ¹H NMR (400 MHz, d₆-DMSO): δ 7.50-7.58 (m, 3H), 7.45 (d, J=7.6 Hz, 2H), 7.27-7.40 (m, 3H), 7.09 (t, J=6.0 Hz, 1H), 3.95 (d, J=7.2 Hz, 2H), 3.09-3.23 (m, 3H), 2.84 (t, J=7.6 Hz, 2H), 2.04-2.11 (m, 1H), 1.17 (d, J=6.8 Hz, 6H), 0.82 (d, J=6.8 Hz, 6H). LCMS (Method A) RT 2.131 min, calcd. for C₂₃H₂₉FN₂O₂S 416.19 m/z, found 417.3 m/z [M+H]⁺.

### Example B-262: N-{2-[6-(2-chlorophenyl)-5-fluoro-1-(2-methylpropyl)-1H-indol-3-yl]ethyl}propane-2-sulfonamide

### Step 1. N-{2-[6-(2-chlorophenyl)-5-fluoro-1-(2-methylpropyl)-1H-indol-3-yl]ethyl}propane-2-sulfonamide

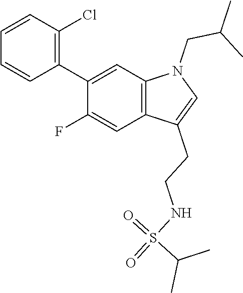 [SMILES: CC(C)Cn1cc(CCNS(=O)(=O)C(C)C)c2cc(F)c(-c3ccccc3Cl)cc21]

To a suspension of N-(2-(6-bromo-5-fluoro-1-isobutyl-1H-indol-3-yl)ethyl)propane-2-sulfonamide (130 mg, 310.00 μmol), 2-(2-chlorophenyl)-4,4,5,5-tetramethyl-1,3,2-dioxaborolane (150 mg, 628.88 μmol) and K₃PO₄·7H₂O (200 mg, 942.21 μmol) in dioxane (3 mL), was added Pd(dppf)Cl₂ (50 mg) under N₂ atmosphere. The reaction mixture was reacted at 85° C. for 30 min and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (26 mg, yield: 18%). ¹H NMR (400 MHz, d₆-DMSO): δ 7.53-7.58 (m, 1H), 7.33-7.44 (m, 5H), 7.32 (s, 1H), 7.10 (s, 1H), 3.91 (d, J=7.2 Hz, 2H), 3.09-3.32 (m, 3H), 2.84 (t, J=7.6 Hz, 2H), 2.00-2.12 (m, 1H), 1.17 (d, J=6.8 Hz, 6H), 0.82 (d, J=6.8 Hz, 6H). LCMS (Method A) RT 2.495 min, calcd. for C₂₃H₂₈ClFN₂O₂S 450.15 m/z, found 451.3 m/z [M+H]⁺.

### Example B-263: N-{2-[6-(3-chloropyridin-4-yl)-5-fluoro-1-(2-methylpropyl)-1H-indol-3-yl]ethyl}propane-2-sulfonamide

### Step 1. N-{2-[6-(3-chloropyridin-4-yl)-5-fluoro-1-(2-methylpropyl)-1H-indol-3-yl]ethyl}propane-2-sulfonamide

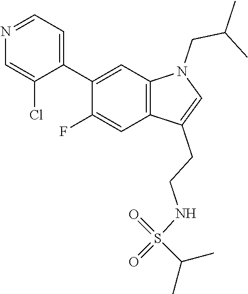 [SMILES: CC(C)Cn1cc(CCNS(=O)(=O)C(C)C)c2cc(F)c(-c3ccncc3Cl)cc21]

To a suspension of N-(2-(6-bromo-5-fluoro-1-isobutyl-1H-indol-3-yl)ethyl)propane-2-sulfonamide (100 mg, 238.46 μmol), 3-chloro-4-(4,4,5,5-tetramethyl-1,3,2-dioxaborolan-2-yl)pyridine (150 mg, 626.29 μmol) and K₃PO₄·7H₂O (300 mg, 1.41 mmol) in dioxane (3 mL), was added Pd(dppf)Cl₂ (100 mg) under N₂ atmosphere. The reaction mixture was reacted at 85° C. for 1 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (26 mg, yield: 24%). ¹H NMR (400 MHz, d₆-DMSO): δ 8.73 (s, 1H), 8.59 (d, J=4.8 Hz, 1H), 7.50-7.55 (m, 2H), 7.43 (d, J=10.8 Hz, 1H), 7.37 (s, 1H), 7.09 (s, 1H), 3.93 (d, J=7.6 Hz, 2H), 3.07-3.32 (m, 3H), 2.82-2.91 (m, 2H), 2.02-2.21 (m, 1H), 1.76 (d, J=6.8 Hz, 6H), 0.82 (d, J=6.8 Hz, 6H). LCMS (Method A) RT 1.912 min, calcd. for C₂₂H₂₇ClFN₃O₂S 451.15 m/z, found 452.1 m/z [M+H]⁺.

### Example B-264: N-{2-[6-(2-chlorophenyl)-5-fluoro-1-(2-methylpropyl)-1H-indol-3-yl]ethyl}propane-2-sulfonamide

### Step 1. N-{2-[6-(2-chlorophenyl)-5-fluoro-1-(2-methylpropyl)-1H-indol-3-yl]ethyl}propane-2-sulfonamide

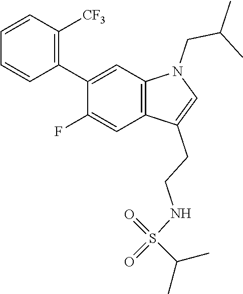 [SMILES: CC(C)Cn1cc(CCNS(=O)(=O)C(C)C)c2cc(F)c(-c3ccccc3C(F)(F)F)cc21]

To a suspension of N-(2-(6-bromo-5-fluoro-1-isobutyl-1H-indol-3-yl)ethyl)propane-2-sulfonamide (100 mg, 238.46 μmol), 2-(trifluoromethyl)phenylboronic acid (100 mg, 526.52 μmol) and K₃PO₄·7H₂O (300 mg, 1.41 mmol) in dioxane (3 mL), was added Pd(dppf)Cl₂ (100 mg) under N₂ atmosphere. The reaction mixture was maintained at 90° C. for 0.5 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (16 mg, yield: 13%). ¹H NMR (400 MHz, d₆-DMSO): δ 7.83 (d, J=7.6 Hz, 1H), 7.72 (t, J=7.6 Hz, 1H), 7.63 (t, J=7.6 Hz, 1H), 7.44 (d, J=7.6 Hz, 1H), 7.29-7.37 (m, 3H), 6.88-7.25 (m, 1H), 3.82-3.96 (m, 2H), 3.05-3.22 (m, 3H), 2.85 (t, J=7.6 Hz, 2H), 1.95-2.08 (m, 1H), 1.15-1.25 (m, 6H), 0.85-0.92 (m, 6H). LCMS (Method A) RT 2.501 min, calcd. for C₂₄H₂₈F₄N₂O₂S 484.18 m/z, found 485.3 m/z [M+H]⁺.

### Example B-265: N-{[5-fluoro-1-(2-methylpropyl)-6-(pyridin-4-yl)-1H-indol-3-yl]methyl}propane-2-sulfonamide

### Step 1. 6-bromo-5-fluoro-1H-indole-3-carbaldehyde

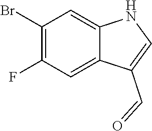 [SMILES: O=Cc1c[nH]c2cc(Br)c(F)cc12]

To a solution of 5-fluoro-6-bromoindole (5.0 g, 23.36 mmol) in DMF (20 mL) in an ice-water bath, was added POCl₃ (7.16 g, 46.72 mmol) dropwise. The mixture was allowed to warm to room temperature and stirred for another 2 h. The resulting mixture was poured into ice-water, quenched with 6 N aqueous NaOH until pH>11 and extracted with EA (3×150 mL). The combined organic layer was washed with brine (3×100 mL), dried over anhydrous Na₂SO₄ and evaporated to give crude product which was washed with ether to give the product as a yellow solid (4.9 g, yield: 86%).

### Step 2. of 6-bromo-5-fluoro-1-isobutyl-1H-indole-3-carbaldehyde

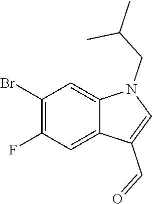 [SMILES: CC(C)Cn1cc(C=O)c2cc(F)c(Br)cc21]

To a solution of 6-bromo-5-fluoro-1H-indole-3-carbaldehyde (4.9 g, 20.24 mmol) and 1-bromo-2-methyl-propane (4.16 g, 30.37 mmol) in DMF (20 mL), were added Cs₂CO₃ (13.19 g, 40.49 mmol) and KI (0.4 g). The mixture was stirred at 80° C. for 5 h and cooled to room temperature. Water (100 mL) and EA (150 mL) was added. The organic layer was separated. The aqueous layer was extracted with EA (2×100 mL). The combined organic layer was washed with brine (4×50 mL), dried over anhydrous Na₂SO₄ and evaporated to give crude product which was purified by silica gel column chromatography to give the product as a yellow oil (5.1 g, yield: 84%).

### Step 3. (E)-6-bromo-5-fluoro-1-isobutyl-1H-indole-3-carbaldehyde O-methyl oxime

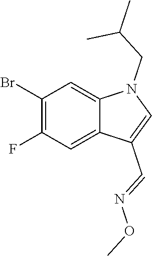 [SMILES: CO/N=C/c1cn(CC(C)C)c2cc(Br)c(F)cc12]

To a suspension of 6-bromo-5-fluoro-1-isobutyl-1H-indole-3-carbaldehyde (2 g, 6.71 mmol) and O-methylhydroxylamine hydrochloride (0.67 g, 8.05 mmol) in H₂O (4 mL) and EtOH (20 mL), was added Na₂CO₃ (499.09 mg, 4.02 mmol). The mixture was stirred at 80° C. for 2 h. The solvent was removed under reduced pressure. The residue was partitioned between EA (50 mL) and water (50 mL). The organic layer was separated. The aqueous layer was extracted with EA (2×50 mL). The combined organic layers were washed with brine (4×50 mL), dried over anhydrous Na₂SO₄ and evaporated to give the product as a yellow solid (2.1 g, 95% yield).

### Step 4. (6-bromo-5-fluoro-1-isobutyl-1H-indol-3-yl)methanamine

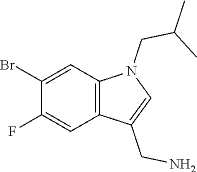 [SMILES: CC(C)Cn1cc(CN)c2cc(F)c(Br)cc21]

A mixture of (E)-6-bromo-5-fluoro-1-isobutyl-1H-indole-3-carbaldehyde O-methyl oxime (2.1 g, 6.42 mmol) and borane-THF (1 N in THF, 13 mL) was refluxed for 2 hr and cooled to room temperature. HCl (2 mL 12 N) was added. The reaction mixture was stirred for another 10 min. The solvent was removed under reduced pressure. The residue was purified by prep-HPLC to give the product as a yellow oil (1.6 g, 83% yield).

### Step 5. N-((6-bromo-5-fluoro-1-isobutyl-1H-indol-3-yl)methyl)propane-2-sulfonamide

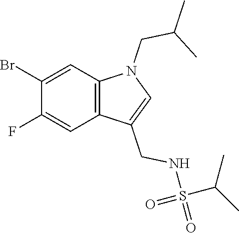 [SMILES: CC(C)Cn1cc(CNS(=O)(=O)C(C)C)c2cc(F)c(Br)cc21]

To a mixture of (6-bromo-5-fluoro-1-isobutyl-1H-indol-3-yl)methanamine (1.6 g, 5.35 mmol) and TEA (1.62 g, 16.04 mmol) in DCM (25 mL), was added propane-2-sulfonyl chloride (1.14 g, 8.02 mmol) dropwise at −40° C. The mixture was allowed to warm to room temperature and stirred for 1 h. The solvent was removed under reduced pressure. The residue was purified by prep-HPLC to give the product as a white solid (0.80 g, yield: 36%).

### Step 6. N-((5-fluoro-1-isobutyl-6-(pyridin-4-yl)-1H-indol-3-yl)methyl)propane-2-sulfonamide

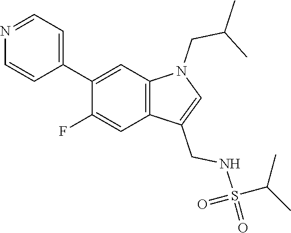 [SMILES: CC(C)Cn1cc(CNS(=O)(=O)C(C)C)c2cc(F)c(-c3ccncc3)cc21]

To a suspension of N-((6-bromo-5-fluoro-1-isobutyl-1H-indol-3-yl)methyl)propane-2-sulfonamide (120 mg, 296.06 μmol), 4-(4,4,5,5-tetramethyl-1,3,2-dioxaborolan-2-yl)pyridine (91.07 mg, 444.09 μmol) and K₃PO₄·7H₂O (300.20 mg, 888.18 μmol) in dioxane (5 mL), was added Pd(dppf)Cl₂ (21.66 mg, 29.61 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (102.53 mg, yield: 85%). ¹H NMR (400 MHz, d₆-DMSO): δ 8.64-8.66 (m, 2H), 7.76 (d, J=6.4 Hz, 1H), 7.61-7.65 (m, 3H), 7.44-7.47 (m, 2H), 4.29 (d, J=6.0 Hz, 2H), 4.03 (d, J=7.2 Hz, 2H), 2.98-3.05 (m, 1H), 2.06-2.17 (m, 1H), 1.16 (d, J=6.8 Hz, 6H), 0.84 (d, J=6.8 Hz, 6H). LCMS (Method A) RT 1.968 min, calcd. for C₂₁H₂₆FN₃O₂S 403.17 m/z, found 404.2 m/z [M+H]⁺.

### Example B-266: N-{[5-fluoro-1-(2-methylpropyl)-6-phenyl-1H-indol-3-yl]methyl}propane-2-sulfonamide

### Step 1. N-((5-fluoro-1-isobutyl-6-phenyl-1H-indol-3-yl)methyl)propane-2-sulfonamide

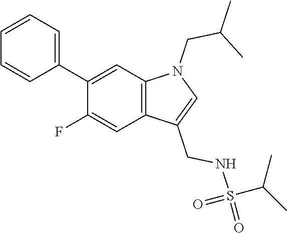 [SMILES: CC(C)Cn1cc(CNS(=O)(=O)C(C)C)c2cc(F)c(-c3ccccc3)cc21]

To a suspension of N-((6-bromo-5-fluoro-1-isobutyl-1H-indol-3-yl)methyl)propane-2-sulfonamide (120 mg, 296.06 μmol), phenylboronic acid (54.15 mg, 444.19 μmol) and K₃PO₄·7H₂O (300.20 mg, 888.18 μmol) in dioxane (5 mL), was added Pd(dppf)Cl₂ (21.66 mg, 29.61 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a light pink solid (76.08 mg, yield: 74%). ¹H NMR (400 MHz, d₆-DMSO): δ 7.54-7.58 (m, 4H), 7.34-7.48 (m, 5H), 4.26 (d, J=6.0 Hz, 2H), 3.98 (d, J=7.6 Hz, 2H), 2.96-3.03 (m, 1H), 2.05-2.12 (m, 1H), 1.15 (d, J=6.8 Hz, 6H), 0.82 (d, J=6.8 Hz, 6H). LCMS (Method A) RT 2.364 min, calcd. for C₂₂H₂₇FN₂O₂S 402.18 m/z, found 403.1 m/z [M+H]⁺.

### Example B-267: N-{[6-(2-chlorophenyl)-5-fluoro-1-(2-methylpropyl)-1H-indol-3-yl]methyl}propane-2-sulfonamide

### Step 1. N-((6-(2-chlorophenyl)-5-fluoro-1-isobutyl-1H-indol-3-yl)methyl)propane-2-sulfonamide

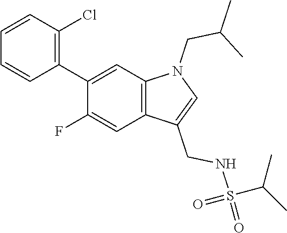 [SMILES: CC(C)Cn1cc(CNS(=O)(=O)C(C)C)c2cc(F)c(-c3ccccc3Cl)cc21]

To a suspension of N-((6-bromo-5-fluoro-1-isobutyl-1H-indol-3-yl)methyl)propane-2-sulfonamide (120 mg, 296.06 μmol), 2-(2-chlorophenyl)-4,4,5,5-tetramethyl-1,3,2-dioxaborolane (105.92 mg, 444.09 μmol) and K₃PO₄·7H₂O (300.20 mg, 888.18 μmol) in dioxane (5 mL), was added Pd(dppf)Cl₂ (21.66 mg, 29.61 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (77 mg, yield: 59%). ¹H NMR (400 MHz, d₆-DMSO): δ 7.52-7.57 (m, 2H), 7.40-7.45 (m, 6H), 4.27 (s, 2H), 3.94 (d, J=7.6 Hz, 2H), 3.00-3.06 (m, 1H), 1.98-2.09 (m, 1H), 1.16 (d, J=6.4 Hz, 6H), 0.81 (d, J=6.8 Hz, 6H). LCMS (Method A) RT 2.439 min, calcd. for C₂₂H₂₆ClFN₂O₂S 436.14 m/z, found 437.2 m/z [M+H]⁺.

### Example B-268: N-{[6-(3-chloropyridin-4-yl)-5-fluoro-1-(2-methylpropyl)-1H-indol-3-yl]methyl}propane-2-sulfonamide

### Step 1. N-((6-(3-chloropyridin-4-yl)-5-fluoro-1-isobutyl-1H-indol-3-yl)methyl)propane-2-sulfonamide

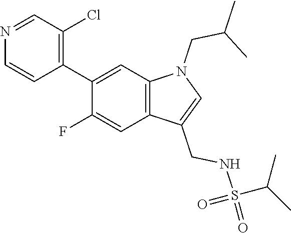 [SMILES: CC(C)Cn1cc(CNS(=O)(=O)C(C)C)c2cc(F)c(-c3ccncc3Cl)cc21]

To a suspension of N-((6-bromo-5-fluoro-1-isobutyl-1H-indol-3-yl)methyl)propane-2-sulfonamide (120 mg, 296.06 μmol), 3-chloro-4-(4,4,5,5-tetramethyl-1,3,2-dioxaborolan-2-yl)pyridine (106.36 mg, 444.09 μmol) and K₃PO₄·7H₂O (300.20 mg, 888.18 μmol) in dioxane (5 mL), was added Pd(dppf)Cl₂ (21.66 mg, 29.61 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (69 mg, yield: 53%). ¹H NMR (400 MHz, d₆-DMSO): δ 8.73 (s, 1H), 8.60 (d, J=4.8 Hz, 1H), 7.56-7.61 (m, 2H), 7.52 (d, J=5.2 Hz, 1H), 7.45 (s, 1H), 7.42 (t, J=6.0 Hz, 1H), 4.28 (d, J=6.0 Hz, 2H), 3.96 (d, J=7.6 Hz, 2H), 3.00-3.07 (m, 1H), 2.03-2.08 (m, 1H), 1.16 (d, J=6.8 Hz, 6H), 0.81 (d, J=6.8 Hz, 6H). LCMS (Method A) RT 2.202 min, calcd. for C₂₁H₂₅ClFN₃O₂S 437.13 m/z, found 438.3 m/z [M+H]⁺.

### Example B-269: N-{[5-fluoro-1-(2-methylpropyl)-6-[2-(trifluoromethyl)phenyl]-1H-indol-3-yl]methyl}propane-2-sulfonamide

### Step 1. N-((5-fluoro-1-isobutyl-6-(2-(trifluoromethyl)phenyl)-1H-indol-3-yl)methyl)propane-2-sulfonamide

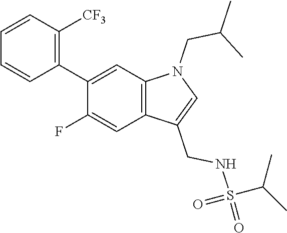 [SMILES: CC(C)Cn1cc(CNS(=O)(=O)C(C)C)c2cc(F)c(-c3ccccc3C(F)(F)F)cc21]

To a suspension of N-((6-bromo-5-fluoro-1-isobutyl-1H-indol-3-yl)methyl)propane-2-sulfonamide (120 mg, 296.06 μmol), 2-(trifluoromethyl)phenylboronic acid (84.38 mg, 444.09 μmol) and K₃PO₄·7H₂O (300.20 mg, 888.18 μmol) in dioxane (5 mL), was added Pd(dppf)Cl₂ (21.66 mg, 29.61 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (79 mg, yield: 56%). ¹H NMR (400 MHz, d₆-DMSO): δ 7.84 (d, J=7.6 Hz, 1H), 7.72 (t, J=7.2 Hz, 1H), 7.63 (t, J=8.0 Hz, 1H), 7.52 (d, J=10.4 Hz, 1H), 7.39-7.46 (m, 4H), 4.27 (d, J=6.0 Hz, 2H), 3.86-3.99 (m, 2H), 2.95-3.02 (m, 1H), 1.98-2.04 (m, 1H), 1.15 (t, J=5.6 Hz, 6H), 0.79 (d, J=6.0 Hz, 6H). LCMS (Method A), RT 2.395 min, calcd. for C₂₃H₂₆F₄N₂O₂S 470.17 m/z, found 471.1 m/z [M+H]⁺.

### Example B-270: N-{2-[1-(3,3-dimethylbutyl)-6-(pyridin-4-yl)-1H-indol-3-yl]ethyl}propane-2-sulfonamide

### Step 1. 6-bromo-1-(3,3-dimethylbutyl)-1H-indole-3-carbaldehyde

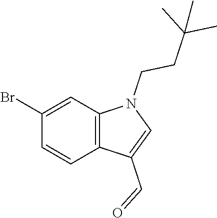 [SMILES: CC(C)(C)CCn1cc(C=O)c2ccc(Br)cc21]

To a solution of 6-bromo-1H-indole-3-carbaldehyde (500 mg, 2.23 mmol) and 1-bromo-3,3-dimethylbutane (368.37 mg, 2.23 mmol) in DMF (10 mL), were added Cs₂CO₃ (1.45 g, 4.46 mmol) and KI (0.1 g). The mixture was stirred at 80° C. for 2 h, and then cooled to room temperature. Water (50 mL) and EA (50 mL) was added. The organic layer was separated. The aqueous layer was extracted with EA (2×50 mL). The combined organic layer was washed with brine (2×50 mL), dried over anhydrous Na₂SO₄ and evaporated to give crude product which was purified by silica gel column chromatography to give the product as a yellow oil (520 mg, yield: 75%).

### Step 2. (E)-6-bromo-1-(3,3-dimethylbutyl)-3-(2-nitrovinyl)-1H-indole

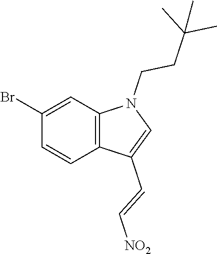 [SMILES: CC(C)(C)CCn1cc(/C=C/[N+](=O)[O-])c2ccc(Br)cc21]

A mixture of 6-bromo-1-(3,3-dimethylbutyl)-1H-indole-3-carbaldehyde (520 mg, 1.69 mmol) and ammonium acetate (260.1 mg, 3.37 mmol) in nitromethane (5 mL) was stirred at 80° C. for 5 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give the product as a yellow oil (520 mg, yield: 87%).

### Step 3. 6-bromo-1-(3,3-dimethylbutyl)-3-(2-nitroethyl)-1H-indole

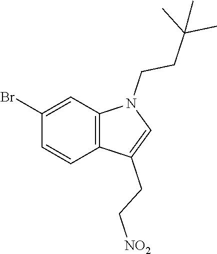 [SMILES: CC(C)(C)CCn1cc(CC[N+](=O)[O-])c2ccc(Br)cc21]

To a solution of (E)-6-bromo-1-(3,3-dimethylbutyl)-3-(2-nitrovinyl)-1H-indole (520 mg, 1.48 mmol) in MeOH (20 mL), was added NaBH₄ (56.01 mg, 1.48 mmol) in portions at 0° C. The mixture was stirred for 1.0 h, and poured into ice-water. The solvent was removed under reduced pressure. The residue was extracted with EA (2×50 mL). The combined organic layer was washed with brine (2×50 mL), dried and evaporated to give crude product which was purified by prep-HPLC to give the product as a yellow solid (450 mg, yield: 86%).

### Step 4. 2-(6-bromo-1-(3,3-dimethylbutyl)-1H-indol-3-yl)ethanamine

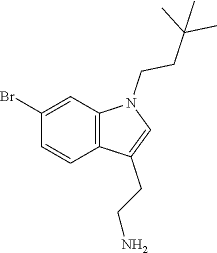 [SMILES: CC(C)(C)CCn1cc(CCN)c2ccc(Br)cc21]

To a solution of 6-bromo-1-(3,3-dimethylbutyl)-3-(2-nitroethyl)-1H-indole (450 mg, 1.27 mmol) and HCl (12 N, 2 mL) in MeOH (4 mL) was added Fe (355.73 mg, 6.37 mmol). The mixture was refluxed for 2 hr. The solid was filtered off and washed with MeOH. The combined organic layer was concentrated and purified by prep-HPLC to give the product as a yellow oil (300 mg, yield: 72%).

### Step 5. N-(2-(6-bromo-1-(3,3-dimethylbutyl)-1H-indol-3-yl)ethyl)propane-2-sulfonamide

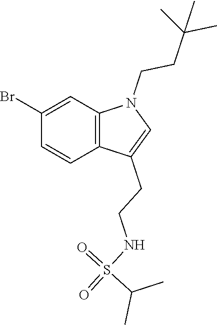 [SMILES: CC(C)S(=O)(=O)NCCc1cn(CCC(C)(C)C)c2cc(Br)ccc12]

To a mixture of 2-(6-bromo-1-(3,3-dimethylbutyl)-1H-indol-3-yl)ethanamine (300 mg, 928.02 μmol) and TEA (281.72 mg, 2.78 mmol) in DCM (5 mL), was added propane-2-sulfonyl chloride (198.51 mg, 1.39 mmol) at −40° C. The mixture was allowed to warm to room temperature and stirred for 1 h. The solvent was removed under reduced pressure. The residue was purified by prep-HPLC to give the product as a yellow oil (120 mg, yield: 30%).

### Step 6. N-(2-(1-(3,3-dimethylbutyl)-6-(pyridin-4-yl)-1H-indol-3-yl)ethyl)propane-2-sulfonamide

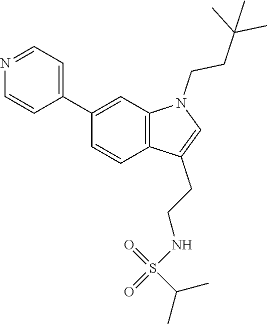 [SMILES: CC(C)S(=O)(=O)NCCc1cn(CCC(C)(C)C)c2cc(-c3ccncc3)ccc12]

To a suspension of N-(2-(6-bromo-1-(3,3-dimethylbutyl)-1H-indol-3-yl)ethyl)propane-2-sulfonamide (120 mg, 279.45 μmol), 4-(4,4,5,5-tetramethyl-1,3,2-dioxaborolan-2-yl)pyridine (85.96 mg, 419.18 μmol) and K₃PO₄·7H₂O (283.36 mg, 419.18 μmol) in dioxane (6 mL), was added Pd(dppf)Cl₂ (20.45 mg, 27.95 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (37 mg, yield: 30%). ¹H NMR (400 MHz, d₆-DMSO): δ 8.58-8.59 (m, 2H), 7.82 (d, J=0.8 Hz, 1H), 7.75-7.76 (m, 2H), 7.62 (d, J=8.4 Hz, 1H), 7.45 (dd, J=8.4 Hz, 1.6 Hz, 1H), 7.36 (s, 1H), 7.11 (br, 1H), 4.17-4.22 (m, 2H), 3.10-3.23 (m, 3H), 2.87 (t, J=7.6 Hz, 2H) 1.64-1.68 (m, 2H), 1.17 (d, J=6.8 Hz, 6H), 0.98 (s, 9H). LCMS (Method A) RT 2.274 min, calcd. for C₂₄H₃₃N₃O₂S 427.23 m/z, found 428.3 m/z [M+H]⁺.

### Example B-271: N-{2-[6-(pyridin-4-yl)-1-[3,3,3-trifluoro-2-(trifluoromethyl)propyl]-1H-indol-3-yl]ethyl}propane-2-sulfonamide

### Step 1. 6-bromo-1-(3,3,3-trifluoro-2-(trifluoromethyl)propyl)-1H-indole-3-carbaldehyde

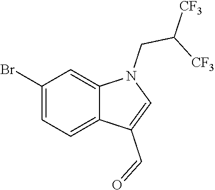 [SMILES: O=Cc1cn(CC(C(F)(F)F)C(F)(F)F)c2cc(Br)ccc12]

To a solution of 6-bromo-1H-indole-3-carbaldehyde (300 mg, 1.34 mmol) and 2-(bromomethyl)-1,1,1,3,3,3-hexafluoropropane (327.99 mg, 1.34 mmol) in CH₃CN (10 mL), was added K₂CO₃ (185.05 mg, 1.34 mmol). The mixture was stirred at 50° C. for 1 h, and then cooled to room temperature. Water (50 mL) and EA (50 mL) was added. The organic layer was separated. The aqueous layer was extracted with EA (2×50 mL). The combined organic layer was washed with brine (2×50 mL), dried over anhydrous Na₂SO₄ and evaporated to give crude product which was purified by prep-TLC to give the product as a yellow oil (300 mg, yield: 57%).

### Step 2. (E)-6-bromo-3-(2-nitrovinyl)-1-(3,3,3-trifluoro-2-(trifluoromethyl)propyl)-1H-indole

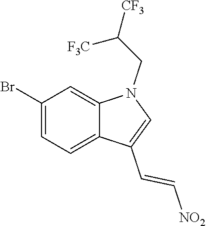 [SMILES: O=[N+]([O-])/C=C/c1cn(CC(C(F)(F)F)C(F)(F)F)c2cc(Br)ccc12]

A mixture of 6-bromo-1-(3,3,3-trifluoro-2-(trifluoromethyl)propyl)-1H-indole-3-carbaldehyde (300 mg, 772.99 μmol) and ammonium acetate (119.17 mg, 1.55 mmol) in nitromethane (5 mL) was stirred at 80° C. for 2 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give the product as a yellow oil (280 mg, yield: 83%).

### Step 3. 6-bromo-3-(2-nitroethyl)-1-(3,3,3-trifluoro-2-(trifluoromethyl)propyl)-1H-indole

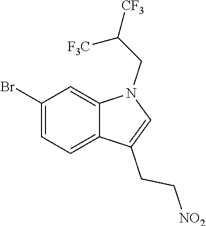 [SMILES: O=[N+]([O-])CCc1cn(CC(C(F)(F)F)C(F)(F)F)c2cc(Br)ccc12]

To a solution of (E)-6-bromo-3-(2-nitrovinyl)-1-(3,3,3-trifluoro-2-(trifluoromethyl)propyl)-1H-indole (280 mg, 649.46 mmol) in MeOH (20 mL), was added NaBH₄ (49.14 mg, 1.30 mmol) in portions at 0° C. The mixture was stirred for 1h at that temperature, and poured into ice-water. The solvent was removed under reduced pressure. The residue was extracted with EA (2×50 mL). The combined organic layer was washed with brine (2×50 mL), dried and evaporated to give crude product which was purified by prep-HPLC to give the product as a yellow oil (260 mg, yield: 82%).

### Step 4. 2-(6-bromo-1-(3,3,3-trifluoro-2-(trifluoromethyl)propyl)-1H-indol-3-yl)ethanamine

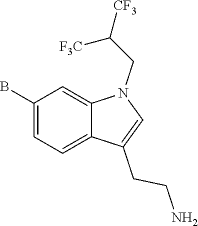

To a solution of 6-bromo-3-(2-nitroethyl)-1-(3,3,3-trifluoro-2-(trifluoromethyl)propyl)-1H-indole (260 mg, 600.26 μmol) and HCl (12 N, 1 mL) in MeOH (4 mL), was added Fe (335.25 mg, 6.00 mmol). The mixture was refluxed for 2 hr. The solid was filtered off and washed with MeOH. The combined organic layer was concentrated and purified by prep-HPLC to give the product as a yellow oil (100 mg, yield: 41%).

### Step 5. N-(2-(6-bromo-1-(3,3,3-trifluoro-2-(trifluoromethyl)propyl)-1H-indol-3-yl)ethyl)propane-2-sulfonamide

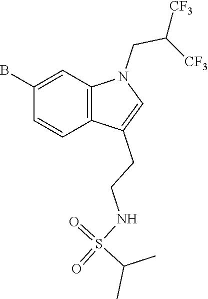

To a mixture of 2-(6-bromo-1-(3,3,3-trifluoro-2-(trifluoromethyl)propyl)-1H-indol-3-yl)ethanamine (180 mg, 446.47 μmol) and TEA (135.54 mg, 1.34 mmol) in DCM (5 mL), was added propane-2-sulfonyl chloride (95.50 mg, 669.71 mmol) at −40° C. The mixture was allowed to warm to room temperature and stirred for 2 h. The solvent was removed under reduced pressure. The residue was purified by prep-HPLC to give the product as a yellow oil (80 mg, yield: 35%).

### Step 6. N-(2-(6-(pyridin-4-yl)-1-(3,3,3-trifluoro-2-(trifluoromethyl)propyl)-1H-indol-3-yl)ethyl)propane-2-sulfonamide

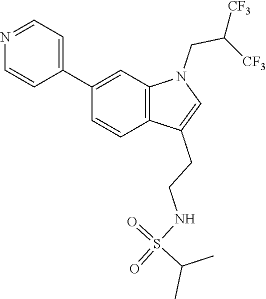 [SMILES: CC(C)S(=O)(=O)NCCc1cn(CC(C(F)(F)F)C(F)(F)F)c2cc(-c3ccncc3)ccc12]

To a suspension of N-(2-(6-bromo-1-(3,3,3-trifluoro-2-(trifluoromethyl)propyl)-1H-indol-3-yl)ethyl)propane-2-sulfonamide (80 mg, 157.08 μmol), 4-(4,4,5,5-tetramethyl-1,3,2-dioxaborolan-2-yl)pyridine (32.21 mg, 157.08 μmol) and K₃PO₄·7H₂O (159.28 mg, 471.23 μmol) in dioxane (5 mL), was added Pd(dppf)Cl₂ (11.49 mg, 15.71 μmol) under N₂ atmosphere. The reaction mixture was reacted at 85° C. for 3 h. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (12.8 mg, yield: 16%). ¹H NMR (400 MHz, d₆-DMSO): δ 8.60-8.62 (m, 2H), 7.90 (s, 1H), 7.77-7.78 (m, 2H), 7.65 (d, J=8.0 Hz, 1H), 7.51 (dd, J=8.4 Hz, 1.2 Hz, 1H), 7.36 (s, 1H), 7.13 (t, J=6.0 Hz, 1H), 4.76-4.83 (m, 3H), 3.10-3.23 (m, 3H), 2.88 (t, J=7.6 Hz, 2H), 1.18 (d, J=6.8 Hz, 6H). LCMS (Method A) RT 2.109 min, calcd. for C₂₂H₂₃F₆N₃O₂S 507.14 m/z, found 508.3 m/z [M+H]⁺.

### Example B-272: N-{2-[1-(cyclopentylmethyl)-6-(pyridin-4-yl)-1H-indol-3-yl]ethyl}propane-2-sulfonamide

### Step 1. 6-bromo-1-(cyclopentylmethyl)-1H-indole-3-carbaldehyde

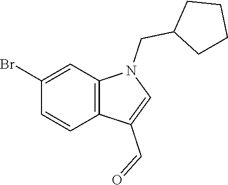 [SMILES: O=Cc1cn(CC2CCCC2)c2cc(Br)ccc12]

To a stirred solution of 6-bromo-1H-indole-3-carbaldehyde (500 mg, 2.24 mmol) in DMF (10 mL) were added (bromomethyl)cyclopentane (548 mg, 3.36 mmol) and K₂CO₃ (464 mg, 3.36 mmol). The resulting reaction mixture was stirred at 70° C. for 1 h and cooled to room temperature. Water (50 mL) and EA (50 mL) was added. The organic layer was separated. The aqueous layer was extracted with EA (2×50 mL). The combined organic layer was washed with brine (2×50 mL), dried over anhydrous Na₂SO₄ and evaporated to give crude product which was used to the next step directly.

### Step 2. (E)-6-bromo-1-(cyclopentylmethyl)-3-(2-nitrovinyl)-1H-indole

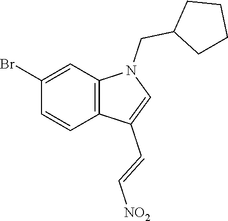 [SMILES: O=[N+]([O-])/C=C/c1cn(CC2CCCC2)c2cc(Br)ccc12]

A mixture of 6-bromo-1-(cyclopentylmethyl)-1H-indole-3-carbaldehyde and ammonium acetate (1 g, 12.97 mmol) in nitromethane (5 mL) was stirred at 80° C. for 1 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give crude product which was used to the next step directly.

### Step 3. 6-bromo-1-(cyclopentylmethyl)-3-(2-nitroethyl)-1H-indole

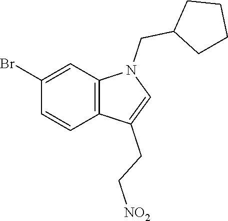 [SMILES: O=[N+]([O-])CCc1cn(CC2CCCC2)c2cc(Br)ccc12]

To a solution of (E)-6-bromo-1-(cyclopentylmethyl)-3-(2-nitrovinyl)-1H-indole in MeOH (10 mL), was added NaBH₄ (400 mg, 10.58 mmol) in portions at 0° C. The mixture was stirred for 1 h at that temperature and poured into ice-water. The solvent was removed under reduced pressure. The residue was extracted with EA (2×50 mL). The combined organic layer was washed with brine (2×50 mL), dried and evaporated to give crude product which was used to the next step directly.

### Step 4. 2-(6-bromo-1-(cyclopentylmethyl)-1H-indol-3-yl)ethanamine

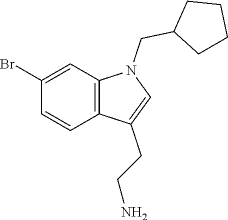 [SMILES: NCCc1cn(CC2CCCC2)c2cc(Br)ccc12]

To a solution of 6-bromo-1-(cyclopentylmethyl)-3-(2-nitroethyl)-1H-indole (500 mg, 1.42 mmol) and HCl (12 N, 5 mL) in MeOH (8 mL), was added Fe (750 mg, 13.43 mmol). The mixture was refluxed for 2 hr. The solid was filtered off and washed with MeOH. The combined organic layer was concentrated and purified by prep-HPLC to give the product as a yellow oil (300 mg, yield: 65%).

### Step 5. N-(2-(6-bromo-1-(cyclopentylmethyl)-1H-indol-3-yl)ethyl)propane-2-sulfonamide

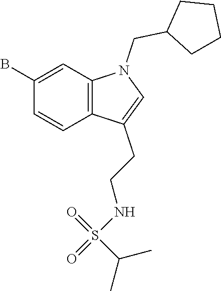

To a solution of 2-(6-bromo-1-(cyclopentylmethyl)-1H-indol-3-yl)ethanamine (300 mg, 0.93 mmol) and DIEA (121 mg, 5.74 mmol) in DCM (3 mL), was added propane-2-sulfonyl chloride (300 mg, 2.10 mmol) dropwise at −20° C. The mixture was stirred for 1h at that temperature. The solvent was removed under reduced pressure. The residue was purified by prep-HPLC to give the product as a white solid (150 mg, yield: 37%).

### Step 6. N-{2-[1-(cyclopentylmethyl)-6-(pyridin-4-yl)-1H-indol-3-yl]ethyl}propane-2-sulfonamide

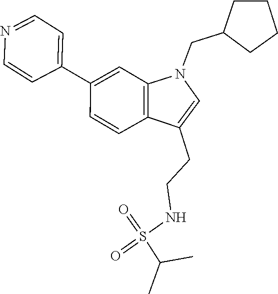 [SMILES: CC(C)S(=O)(=O)NCCc1cn(CC2CCCC2)c2cc(-c3ccncc3)ccc12]

To a suspension of N-(2-(6-bromo-1-(cyclopentylmethyl)-1H-indol-3-yl)ethyl)propane-2-sulfonamide (150 mg, 350.96 μmol), 4-(4,4,5,5-tetramethyl-1,3,2-dioxaborolan-2-yl)pyridine (150 mg, 731.49 μmol) and K₃PO₄·7H₂O (250 mg, 1.18 mmol) in dioxane (3 mL), was added Pd(dppf)Cl₂ (50 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (8 mg, yield: 5%). ¹HNMR (400 MHz, d₆-DMSO) δ 8.58 (d, J=4.4 Hz, 2H), 7.92 (d, J=1.2 Hz, 1H), 7.77 (d, J=4.4 Hz, 2H), 7.61 (d, J=8.4 Hz, 1H), 7.45 (d, J=8.4 Hz, 1H), 7.34 (s, 1H), 7.01-7.22 (m, 1H), 4.11 (d, J=7.6 Hz, 2H), 3.10-3.24 (m, 3H), 2.87 (t, J=7.6 Hz, 2H), 2.28-2.42 (m, 1H), 1.54-1.65 (m, 6H), 1.20-1.28 (m, 2H), 1.17 (d, J=6.8 Hz, 6H). LCMS (Method B) RT 2.305 min, calcd. for C₂₄H₃₁N₃O₂S 425.21 m/z, found 426.3 m/z [M+H]⁺.

### Example B-273: N-{1,1,1-trifluoro-3-[1-(2-methylpropyl)-6-(pyridin-4-yl)-1H-indol-3-yl]propan-2-yl}propane-2-sulfonamide

### Step 1. 3-(6-bromo-1-isobutyl-1H-indol-3-yl)-1,1,1-trifluoropropan-2-one

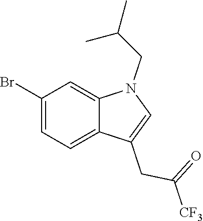 [SMILES: CC(C)Cn1cc(CC(=O)C(F)(F)F)c2ccc(Br)cc21]

To a solution of 2-(6-bromo-1-isobutyl-1H-indol-3-yl)acetic acid (1.1 g, 3.55 mmol) in DCM (15 mL), were added (COCl₂)₂ (900.23 mg, 7.09 mmol) and DMF (25.92 mg, 354.63 μmol). The mixture was stirred for 1 h. The solvent was removed under reduced pressure. The residue was dissolved in DCM (50 mL). Pyridine (561.02 mg, 7.09 mmol) and TFAA (2.23 g, 10.64 mmol) was added at −60° C. The mixture was allowed to warm to room temperature and stirred for 10 h. Water (25 mL) was added. The organic layer was separated, washed with brine (25 mL), dried over anhydrous Na₂SO₄ and evaporated to give crude product which was purified by prep-HPLC to give the product as a yellow oil (500 mg, yield: 38%).

### Step 2. (E)-3-(6-bromo-1-isobutyl-1H-indol-3-yl)-1,1,1-trifluoropropan-2-one O-methyl oxime

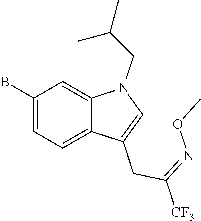

To a suspension of 3-(6-bromo-1-isobutyl-1H-indol-3-yl)-1,1,1-trifluoropropan-2-one (500 mg, 1.38 mmol) and O-methylhydroxylamine hydrochloride (230.59 mg, 2.76 mmol) in H₂O (2 mL) and EtOH (10 mL), was added Na₂CO₃ (146.32 mg, 1.38 mmol). The mixture was stirred at 80° C. for 2 h. The solvent was removed under reduced pressure. The residue was partitioned between EA (50 mL) and water (50 mL). The organic layer was separated. The aqueous layer was extracted with EA (2×50 mL). The combined organic layer was washed with brine (2×50 mL), dried over anhydrous Na₂SO₄ and evaporated to give the product as a yellow solid (540.09 mg, yield: 100%).

### Step 3. 3-(6-bromo-1-isobutyl-1H-indol-3-yl)-1,1,1-trifluoropropan-2-amine

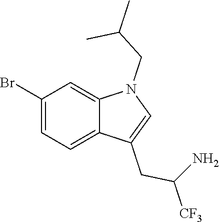 [SMILES: CC(C)Cn1cc(CC(N)C(F)(F)F)c2ccc(Br)cc21]

A mixture of (E)-3-(6-bromo-1-isobutyl-1H-indol-3-yl)-1,1,1-trifluoropropan-2-one O-methyl oxime (540.09 mg, 1.38 mmol) and borane-THF (1 N in THF, 13 mL) was refluxed for 2 hr and cooled to room temperature. HCl (2 mL, 12N aqueous) was added. The reaction mixture was stirred for another 10 min. The solvent was removed under reduced pressure. The residue was purified by prep-HPLC to give the product as a yellow oil (300 mg, yield: 59%).

### Step 4. N-(3-(6-bromo-1-isobutyl-1H-indol-3-yl)-1,1,1-trifluoropropan-2-yl)propane-2-sulfonamide

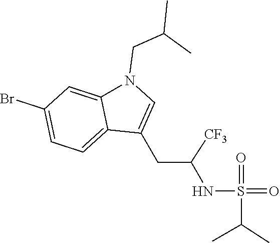 [SMILES: CC(C)Cn1cc(CC(NS(=O)(=O)C(C)C)C(F)(F)F)c2ccc(Br)cc21]

To a mixture of 3-(6-bromo-1-isobutyl-1H-indol-3-yl)-1,1,1-trifluoropropan-2-amine (460 mg, 1.27 mmol) and DBU (1.93 mg, 12.66 mmol) in DCM (4 mL), was added propane-2-sulfonyl chloride (361.21 mg, 2.53 mmol) at −40° C. The mixture was allowed to warm to room temperature and stirred for 2 h. The solvent was removed under reduced pressure. The residue was purified by prep-TLC to give the product as a yellow oil (260 mg, yield: 43%).

### Step 5. N-(1,1,1-trifluoro-3-(1-isobutyl-6-(pyridin-4-yl)-1H-indol-3-yl)propan-2-yl)propane-2-sulfonamide

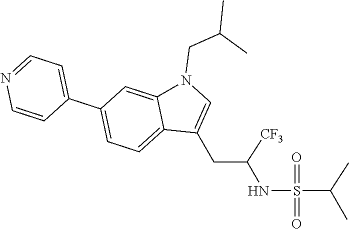 [SMILES: CC(C)Cn1cc(CC(NS(=O)(=O)C(C)C)C(F)(F)F)c2ccc(-c3ccncc3)cc21]

To a suspension of N-(3-(6-bromo-1-isobutyl-1H-indol-3-yl)-1,1,1-trifluoropropan-2-yl)propane-2-sulfonamide (120 mg, 255.67 μmol), 4-(4,4,5,5-tetramethyl-1,3,2-dioxaborolan-2-yl)pyridine (68.16 mg, 332.37 μmol) and K₃PO₄·7H₂O (259.25 mg, 767.00 μmol) in dioxane (5 mL), was added Pd(dppf)Cl₂ (18.71 mg, 25.57 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (81 mg, yield: 67%). ¹H NMR (400 MHz, d₆-DMSO): δ 8.60-8.61 (m, 2H), 8.06 (d, J=9.2 Hz, 1H), 7.96 (s, 1H), 7.79-7.81 (m, 2H), 7.70 (d, J=8.4 Hz, 1H), 7.50 (dd, J=8.4 Hz, 1.2 Hz, 1H), 7.41 (s, 1H), 3.98-4.24 (m, 3H), 3.27-3.32 (m, 1H), 3.18 (dd, J=14.8 Hz, 4.0 Hz, 1H), 2.96-3.02 (m, 1H), 2.09-2.16 (m, 1H), 0.98 (d, J=6.8 Hz, 3H), 0.84-0.90 (m, 9H). LCMS (Method A) RT 2.235 min, calcd. for C₂₃H₂₈F₃N₃O₂S 467.19 m/z, found 468.3 m/z [M+H]⁺.

### Example B-274: N-methyl-N-{1,1,1-trifluoro-3-[1-(2-methylpropyl)-6-(pyridin-4-yl)-1H-indol-3-yl]propan-2-yl}propane-2-sulfonamide

### Step 1. N-(3-(6-bromo-1-isobutyl-1H-indol-3-yl)-1,1,1-trifluoropropan-2-yl)-N-methylpropane-2-sulfonamide

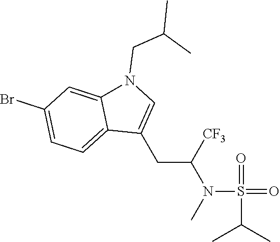 [SMILES: CC(C)Cn1cc(CC(N(C)S(=O)(=O)C(C)C)C(F)(F)F)c2ccc(Br)cc21]

To a solution of N-(3-(6-bromo-1-isobutyl-1H-indol-3-yl)-1,1,1-trifluoropropan-2-yl)propane-2-sulfonamide (140 mg, 298.28 mmol) in DMF (5 mL), was added NaH (60% in mineral) (23.86 mg, 596.56 mmol) in portions at 0° C. The mixture was stirred for 1 h, and then Mel (63.51 mg, 447.42 mmol) was added dropwise. The reaction mixture was stirred for another 2 h and poured in to ice-water (20 mL). The organic layer was separated. The aqueous layer was extracted with EA (2×20 mL). The combined organic layer was washed with brine (2×20 mL), dried over anhydrous Na₂SO₄ and evaporated to give the product as a white solid (120 mg, yield: 83%).

### Step 2. N-methyl-N-{1,1,1-trifluoro-3-[1-(2-methylpropyl)-6-(pyridin-4-yl)-1H-indol-3-yl]propan-2-yl}propane-2-sulfonamide

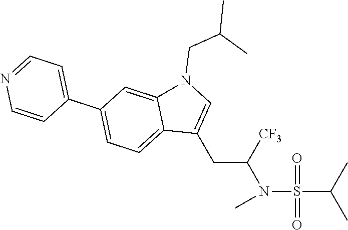 [SMILES: CC(C)Cn1cc(CC(N(C)S(=O)(=O)C(C)C)C(F)(F)F)c2ccc(-c3ccncc3)cc21]

To a suspension of N-(3-(6-bromo-1-isobutyl-1H-indol-3-yl)-1,1,1-trifluoropropan-2-yl)-N-methylpropane-2-sulfonamide (120 mg, 248.25 μmol), 4-(4,4,5,5-tetramethyl-1,3,2-dioxaborolan-2-yl)pyridine (50.91 mg, 248.25 μmol) and K₃PO₄·7H₂O (251.72 mg, 744.75 μmol) in dioxane (5 mL), was added Pd(dppf)Cl₂ (18.16 mg, 24.82 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (68 mg, yield: 56%). ¹H NMR (400 MHz, d₆-DMSO): δ 8.60-8.61 (m, 2H), 7.97 (d, J=1.2 Hz, 1H), 7.79-7.81 (m, 2H), 7.71 (d, J=8.0 Hz, 1H), 7.50-7.52 (m, 2H), 4.72-4.78 (m, 1H), 3.99-4.10 (m, 2H), 3.22-3.35 (m, 2H), 2.92 (s, 3H), 2.76-2.82 (m, 1H), 2.09-2.16 (m, 1H), 0.87-0.90 (m, 9H), 0.77 (d, J=6.8 Hz, 3H). LCMS (Method A) RT 2.367 min, calcd. for C₂₄H₃₀F₃N₃O₂S 481.20 m/z, found 482.4 m/z [M+H]⁺.

### Example B-275: N-{1-[1-(2-methylpropyl)-6-(pyridin-4-yl)-1H-indol-3-yl]propan-2-yl}propane-2-sulfonamide

### Step 1. (E)-6-bromo-1-isobutyl-3-(2-nitroprop-1-enyl)-1H-indole

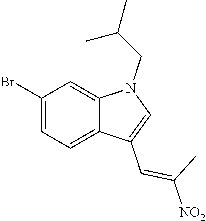 [SMILES: C/C(=C\c1cn(CC(C)C)c2cc(Br)ccc12)[N+](=O)[O-]]

A mixture of 6-bromo-1-isobutyl-1H-indole-3-carbaldehyde (2 g, 7.14 mmol) and ammonium acetate (1.1 g, 14.28 mmol) in nitroethane (15 mL) was stirred at 70° C. for 1 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give the product as a yellow oil (2.1 g, yield: 87%).

### Step 2. 6-bromo-1-isobutyl-3-(2-nitropropyl)-1H-indole

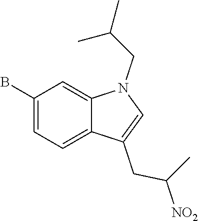

To a solution of (E)-6-bromo-1-isobutyl-3-(2-nitroprop-1-enyl)-1H-indole (2.1 g, 6.23 mmol) in MeOH (20 mL), was added NaBH₄ (471.18 mg, 12.46 mmol) in portions at 0° C. The mixture was stirred for 1 h, and poured into ice-water. The solvent was removed under reduced pressure. The residue was extracted with EA (2×50 mL). The combined organic layer was washed with brine (2×50 mL), dried and evaporated to give crude product which was purified by prep-HPLC to give the product as a yellow solid (450 mg, yield: 89%

### Step 3. 1-(6-bromo-1-isobutyl-1H-indol-3-yl)propan-2-amine

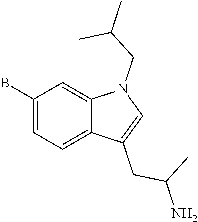

To a solution of 6-bromo-1-isobutyl-3-(2-nitropropyl)-1H-indole (1.9 g, 5.60 mmol) and 12 N aqueous HCl (2 mL) in MeOH (8 mL) was added Fe (1.56 g, 28.00 mmol). The mixture was refluxed for 2 hr. The solid was filtered off and washed with MeOH. The combined organic layer was concentrated and purified by prep-HPLC to give the product as a yellow oil (900 mg, yield: 51%).

### Step 4. N-(1-(6-bromo-1-isobutyl-1H-indol-3-yl)propan-2-yl)propane-2-sulfonamide

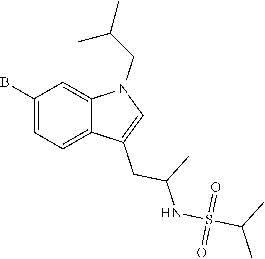

To a mixture of 1-(6-bromo-1-isobutyl-1H-indol-3-yl)propan-2-amine (900 mg, 2.91 mmol) and TEA (294.50 mg, 2.91 mmol) in DCM (15 mL), was added propane-2-sulfonyl chloride (830.05 mg, 5.82 mmol) at 0° C. The mixture was allowed to warm to room temperature and stirred for 1 h. The solvent was removed under reduced pressure. The residue was purified by prep-HPLC to give the product as a yellow oil (350 mg, yield: 28%).

### Step 5. N-{1-[1-(2-methylpropyl)-6-(pyridin-4-yl)-1H-indol-3-yl]propan-2-yl}propane-2-sulfonamide

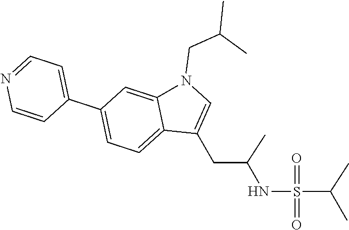 [SMILES: CC(C)Cn1cc(CC(C)NS(=O)(=O)C(C)C)c2ccc(-c3ccncc3)cc21]

To a suspension of N-(1-(6-bromo-1-isobutyl-1H-indol-3-yl)propan-2-yl)propane-2-sulfonamide (120 mg, 288.89 μmol), 4-(4,4,5,5-tetramethyl-1,3,2-dioxaborolan-2-yl)pyridine (88.86 mg, 433.33 μmol) and K₃PO₄·7H₂O (292.93 mg, 866.66 μmol) in dioxane (6 mL), was added Pd(dppf)Cl₂ (21.14 mg, 28.89 μmol) under N₂ atmosphere. The reaction mixture was reacted at 85° C. for 3 h. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (32 mg, yield: 26%). ¹H NMR (400 MHz, d₆-DMSO): δ 8.57-8.58 (m, 2H), 7.90 (d, J=0.8 Hz, 1H), 7.76-7.77 (m, 2H), 7.65 (d, J=8.4 Hz, 1H), 7.45 (dd, J=8.4 Hz, 1.6 Hz, 1H), 7.28 (s, 1H), 7.00 (br, 1H), 3.96-4.07 (m, 2H), 3.51-3.58 (m, 1H), 2.92-2.98 (m, 2H), 2.69-2.74 (m, 1H), 2.07-2.16 (m, 1H), 1.06-1.13 (m, 9H), 0.83-0.86 (m, 6H). LCMS (Method A) RT 2.169 min, calcd. for C₂₃H₃₁N₃O₂S 413.21 m/z, found 414.4 m/z [M+H]⁺.

### Example B-276: N-methyl-N-{1-[1-(2-methylpropyl)-6-(pyridin-4-yl)-1H-indol-3-yl]propan-2-yl}propane-2-sulfonamide

### Step 1. N-(1-(6-bromo-1-isobutyl-1H-indol-3-yl)propan-2-yl)-N-methylpropane-2-sulfonamide

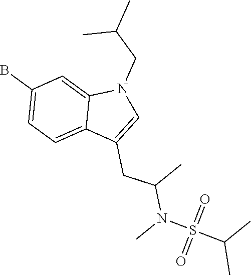

To a solution of N-(1-(6-bromo-1-isobutyl-1H-indol-3-yl)propan-2-yl)propane-2-sulfonamide (200 mg, 481.48 mmol) in DMF (5 mL), was added NaH (60% in mineral) (37.08 mg, 962.96 mmol) in portions at 0° C. The mixture was stirred for 1 h, and then Mel (102.51 mg, 722.22 mmol) was added dropwise. The reaction mixture was stirred for another 1 h and poured in to ice-water (20 mL). The organic layer was separated. The aqueous layer was extracted with EA (2×20 mL). The combined organic layer was washed with brine (2×20 mL), dried over anhydrous Na₂SO₄ and evaporated to give the product as a white solid (206.76 mg, yield: 100%).

### Step 2. N-(1-(1-isobutyl-6-(pyridin-4-yl)-1H-indol-3-yl)propan-2-yl)-N-methylpropane-2-sulfonamide

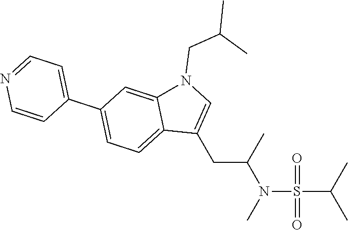 [SMILES: CC(C)Cn1cc(CC(C)N(C)S(=O)(=O)C(C)C)c2ccc(-c3ccncc3)cc21]

To a suspension of N-(1-(6-bromo-1-isobutyl-1H-indol-3-yl)propan-2-yl)-N-methylpropane-2-sulfonamide (210 mg, 490.65 μmol), 4-(4,4,5,5-tetramethyl-1,3,2-dioxaborolan-2-yl)pyridine (120.70 mg, 588.78 μmol) and K₃PO₄·7H₂O (331.24 mg, 981.30 μmol) in dioxane (5 mL), was added Pd(dppf)Cl₂ (18.16 mg, 24.82 μmol) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (150 mg, yield: 71%). ¹H NMR (400 MHz, d₆-DMSO): δ 8.57-8.59 (m, 2H), 7.91 (d, J=1.2 Hz, 1H), 7.76-7.78 (m, 2H), 7.68 (d, J=8.4 Hz, 1H), 7.46 (dd, J=8.0 Hz, 1.2 Hz, 1H), 7.31 (s, 2H), 3.96-4.12 (m, 3H), 2.98-3.05 (m, 1H), 2.91 (d, J=7.6 Hz, 2H), 3.12 (s, 3H), 2.07-2.17 (m, 1H), 2.11 (d, J=6.4 Hz, 3H), 1.06 (d, J=6.8 Hz, 3H), 0.99 (d, J=6.8 Hz, 3H), 0.83-0.86 (m, 6H). LCMS (Method A) RT 2.302 min, calcd. for C₂₄H₃₃N₃O₂S 427.23 m/z, found 428.4 m/z [M+H]⁺.

### Example B-277: N-{2-[1-(2-methylpropyl)-6-(pyridin-4-yl)-1H-indol-3-yl]ethyl}-N-(propan-2-yl)propane-2-sulfonamide

### Step 1. (E)-6-bromo-1-isobutyl-3-(2-nitrovinyl)-1H-indole

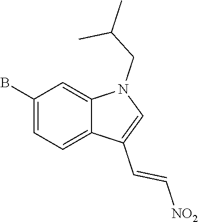

A mixture of 6-bromo-1-isobutyl-1H-indole-3-carbaldehyde (1.0 g, 3.58 mmol) and ammonium acetate (1.0 g, 13.01 mmol) in nitromethane (5 mL) was stirred at 80° C. for 1 hr. The solvent was removed under reduced pressure. The residue was partitioned between EA (50 mL) and water (50 mL). The organic layer was separated. The aqueous layer was extracted with EA (2×50 mL). The combined organic layer was washed with brine (3×50 mL), dried over anhydrous Na₂SO₄ and evaporated to give crude product which was used to the next step directly.

### Step 2. 6-bromo-1-isobutyl-3-(2-nitroethyl)-1H-indole

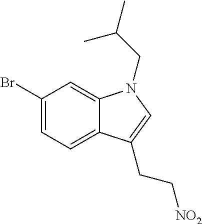 [SMILES: CC(C)Cn1cc(CC[N+](=O)[O-])c2ccc(Br)cc21]

To a solution of (E)-6-bromo-1-isobutyl-3-(2-nitrovinyl)-1H-indole (1.0 g, 3.11 mmol) in MeOH (10 mL), was added NaBH₄ (800 mg, 21.16 mmol) in portions at 0° C. The mixture was stirred for 1h at that temperature and poured into ice-water. The solvent was removed under reduced pressure. The residue was extracted with EA (2×50 mL). The combined organic layer was washed with brine (2×50 mL), dried and evaporated to give crude product which was used to the next step directly.

### Step 3. 2-(6-bromo-1-isobutyl-1H-indol-3-yl)ethan-1-amine

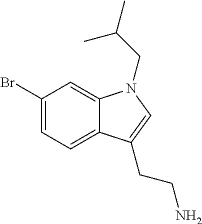 [SMILES: CC(C)Cn1cc(CCN)c2ccc(Br)cc21]

To a solution of 6-bromo-1-isobutyl-3-(2-nitroethyl)-1H-indole (1 g, 3.08 mmol) and HCl (12 N, 10 mL) in MeOH (15 mL), was added Fe (1.5 mg, 26.86 mmol). The mixture was refluxed for 2 hr. The solid was filtered off and washed with MeOH. The combined organic layer was concentrated and purified by prep-HPLC to give the product as a yellow solid (700 mg, yield: 77%).

### Step 4. N-(2-(6-bromo-1-isobutyl-1H-indol-3-yl)ethyl)propane-2-sulfonamide

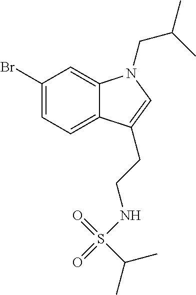 [SMILES: CC(C)Cn1cc(CCNS(=O)(=O)C(C)C)c2ccc(Br)cc21]

To a solution of 2-(6-bromo-1-isobutyl-1H-indol-3-yl)ethan-1-amine (700 mg, 2.37 mmol) and DIEA (306 mg, 2.37 mmol) in DCM (3 mL), was added propane-2-sulfonyl chloride (700 mg, 4.91 mmol) dropwise at −20° C. The mixture was stirred for 1 h at that temperature. The solvent was removed under reduced pressure. The residue was purified by prep-HPLC to give the product as a white solid (200 mg, yield: 21%).

### Step 5. N-(2-(6-bromo-1-isobutyl-1H-indol-3-yl)ethyl)-N-isopropylpropane-2-sulfonamide

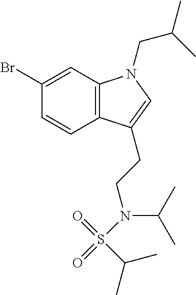 [SMILES: CC(C)Cn1cc(CCN(C(C)C)S(=O)(=O)C(C)C)c2ccc(Br)cc21]

To a solution of N-(2-(6-bromo-1-isobutyl-1H-indol-3-yl)ethyl)propane-2-sulfonamide (200 mg, 0.50 mmol) in DMF (3 mL), was added NaH (60% in mineral) (80.00 mg, 2.00 mmol) in portions at 0° C. The mixture was stirred for 1 h, and then 2-iodopropane (680.00 mg, 4.00 mmol) was added dropwise. The reaction mixture was stirred for another 4 h and poured in to ice-water (20 mL). The organic layer was separated. The aqueous layer was extracted with EA (2×20 mL). The combined organic layer was washed with brine (2×20 mL), dried over anhydrous Na₂SO₄ and evaporated to give the product as a white solid (80 mg, yield: 36%).

### Step 6. N-{2-[1-(2-methylpropyl)-6-(pyridin-4-yl)-1H-indol-3-yl]ethyl}-N-(propan-2-yl)propane-2-sulfonamide

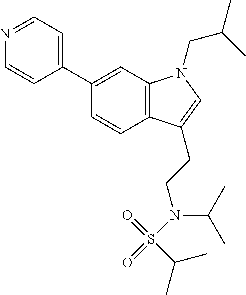 [SMILES: CC(C)Cn1cc(CCN(C(C)C)S(=O)(=O)C(C)C)c2ccc(-c3ccncc3)cc21]

To a suspension of N-(2-(6-bromo-1-isobutyl-1H-indol-3-yl)ethyl)-N-isopropylpropane-2-sulfonamide (80 mg, 180.41 μmol), 4-(4,4,5,5-tetramethyl-1,3,2-dioxaborolan-2-yl)pyridine (80 mg, 390.13 μmol) and K₃PO₄·7H₂O (150 mg, 706.66 μmol) in dioxane (3 mL), was added Pd(dppf)Cl₂ (20 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (11 mg, yield: 13%).

¹HNMR (400 MHz, d₆-DMSO) δ 8.58 (d, J=4.4 Hz, 2H), 7.91 (s, 1H), 7.77 (d, J=6.0 Hz, 2H), 7.65 (d, J=4.4 Hz, 1H), 7.46 (d, J=8.4 Hz, 1H), 7.33 (s, 1H), 4.01 (d, J=7.2 Hz, 2H), 3.85-3.95 (m, 1H), 3.27-3.29 (m, 2H), 2.90-2.99 (m, 2H), 2.07-2.20 (m, 1H), 1.15-1.24 (m, 12H), 0.85 (d, J=6.4 Hz, 6H). LCMS (Method B), RT 2.372 min, calcd. for C₂₅H₃₅N₃O₂S 441.24 m/z, found 442.3 m/z [M+H]⁺.

### Example B-278: N-{[1-(2,2-dimethylpropyl)-5-fluoro-6-[2-(trifluoromethyl)phenyl]-1H-indol-3-yl]methyl}cyclopropanesulfonamide

### Step 1. 6-bromo-5-fluoro-1-neopentyl-1H-indole-3-carbaldehyde

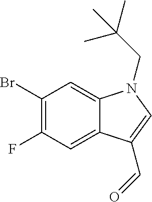 [SMILES: CC(C)(C)Cn1cc(C=O)c2cc(F)c(Br)cc21]

To a solution of 6-bromo-5-fluoro-1H-indole-3-carbaldehyde (800 mg, 3.32 mmol) and 1-bromo-2,2-dimethylpropane (747 mg, 4.98 mmol) in DMF (10 mL), were added Cs₂CO₃ (2.18 g, 6.64 mmol) and KI (0.1 g). The mixture was stirred at 80° C. for 1 h and cooled to room temperature. Water (50 mL) and EA (50 mL) was added. The organic layer was separated. The aqueous layer was extracted with EA (2×30 mL). The combined organic layer was washed with brine (2×30 mL), dried over anhydrous Na₂SO₄ and evaporated to give crude product which was purified by silica gel column chromatography to give the product as a yellow oil (420 mg, yield: 40%).

### Step 2. ((E)-6-bromo-5-fluoro-1-neopentyl-1H-indole-3-carbaldehyde O-methyl oxime

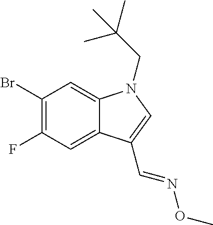 [SMILES: CO/N=C/c1cn(CC(C)(C)C)c2cc(Br)c(F)cc12]

To a suspension of. 6-bromo-5-fluoro-1-neopentyl-1H-indole-3-carbaldehyde (0.42 g, 1.35 mmol) and O-methylhydroxylamine hydrochloride (0.22 g, 2.70 mmol) in H₂O (4 mL) and EtOH (20 mL), was added Na₂CO₃ (143.1 mg, 1.35 mmol). The mixture was refluxed for 2 h. The solvent was removed under reduced pressure. The residue was partitioned between EA (50 mL) and water (50 mL). The organic layer was separated. The aqueous layer was extracted with EA (2×50 mL). The combined organic layers were washed with brine (2×50 mL), dried over anhydrous Na₂SO₄ and evaporated to give the product as a yellow solid (0.45 g, 98% yield).

### Step 3. (6-bromo-5-fluoro-1-neopentyl-1H-indol-3-yl)methanamine

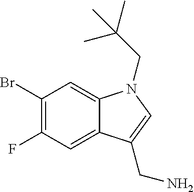 [SMILES: CC(C)(C)Cn1cc(CN)c2cc(F)c(Br)cc21]

A mixture of ((E)-6-bromo-5-fluoro-1-neopentyl-1H-indole-3-carbaldehyde O-methyl oxime (0.45 g, 1.44 mmol) and borane-THF (1 N in THF, 5 mL) was refluxed for 1 hr and cooled to room temperature. HCl (1 mL, 12 N aqueous) was added. The reaction mixture was stirred for another 10 min. The solvent was removed under reduced pressure. The residue was purified by prep-HPLC to give the product as a yellow oil (0.35 g, 84% yield).

### Step 4. N-((6-bromo-5-fluoro-1-neopentyl-1H-indol-3-yl)methyl)cyclopropanesulfonamide

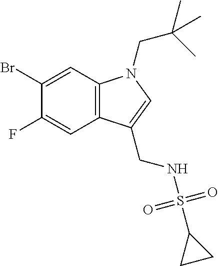 [SMILES: CC(C)(C)Cn1cc(CNS(=O)(=O)C2CC2)c2cc(F)c(Br)cc21]

To a mixture of (6-bromo-5-fluoro-1-neopentyl-1H-indol-3-yl)methanamine (0.35 g, 1.12 mmol) and TEA (0.34 g, 3.36 mmol) in DCM (25 mL), was added cyclopropanesulfonyl chloride (188.16 mg, 1.34 mmol) dropwise at room temperature. The mixture was stirred for 2 h. The solvent was removed under reduced pressure. The residue was purified by prep-HPLC to give the product as a white solid (280 mg, yield: 60%).

### Step 5. N-((5-fluoro-1-neopentyl-6-(2-(trifluoromethyl)phenyl)-1H-indol-3-yl)methyl)

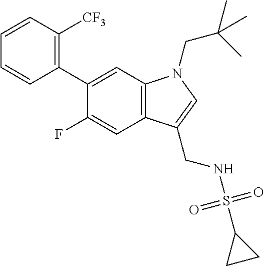 [SMILES: CC(C)(C)Cn1cc(CNS(=O)(=O)C2CC2)c2cc(F)c(-c3ccccc3C(F)(F)F)cc21]

To a suspension of N-((6-bromo-5-fluoro-1-neopentyl-1H-indol-3-yl)methyl)cyclopropanesulfonamide (100 mg, 0.24 mmol), 2-(trifluoromethyl)phenylboronic acid (54.72 mg, 0.29 mmol) and K₃PO₄·7H₂O (236.16 mg, 0.72 mmol) in dioxane (5 mL), was added Pd(dppf)Cl₂ (20 mg) under N₂ atmosphere. The reaction mixture was reacted at 85° C. for 3 h and cooled to room temperature. The resulting reaction mixture was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (42 mg, yield: 36%). ¹H NMR (400 MHz, d₆-DMSO): δ 7.76 (d, J=7.6 Hz, 1H), 7.66 (t, J=7.2 Hz, 1H), 7.57 (d, J=7.6 Hz, 1H), 7.35-7.42 (m, 4H), 7.30 (s, 1H), 4.25 (d, J=6.0 Hz, 2H), 3.95 (d, J=14.8 Hz, 1H), 3.79 (d, J=14.4 Hz, 1H), 2.36-2.44 (m, 1H), 0.74-0.84 (m, 13H). LCMS (Method A) RT 2.459 min, calcd. for C₂₄H₂₆F₄N₂O₂S 482.17 m/z, found 483.3 m/z [M+H]⁺.

### Example B-279: N-{[6-(3-chloropyridin-4-yl)-1-(2,2-dimethylpropyl)-5-fluoro-1H-indol-3-yl]methyl}cyclopropanesulfonamide

### Step 1. N-{[6-(3-chloropyridin-4-yl)-1-(2,2-dimethylpropyl)-5-fluoro-1H-indol-3-yl]methyl}cyclopropanesulfonamide

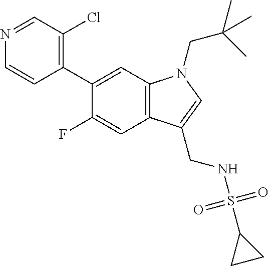 [SMILES: CC(C)(C)Cn1cc(CNS(=O)(=O)C2CC2)c2cc(F)c(-c3ccncc3Cl)cc21]

To a suspension of N-((6-bromo-5-fluoro-1-neopentyl-1H-indol-3-yl)methyl)cyclopropanesulfonamide (100 mg, 0.24 mmol), 3-chloro-4-(4,4,5,5-tetramethyl-1,3,2-dioxaborolan-2-yl)pyridine (69.31 mg, 0.29 mmol) and K₃PO₄·7H₂O (236.16 mg, 0.72 mmol) in dioxane (5 mL), was added Pd(dppf)Cl₂ (20 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (36 mg, yield: 33%).

¹H NMR (400 MHz, d₆-DMSO): δ 8.74 (s, 1H), 8.60 (d, J=5.2 Hz, 1H), 7.60 (d, J=6.0 Hz, 1H), 7.55 (d, J=10.8 Hz, 1H), 7.42-7.48 (m, 3H), 4.32 (d, J=6.0 Hz, 2H), 3.97 (s, 2H), 2.45-2.50 (m, 1H), 0.83-0.92 (m, 13H). LCMS (Method A) RT 2.236 min, calcd. for C₂₂H₂₅ClFN₃O₂S 449.13 m/z, found 450.4 m/z [M+H]⁺.

### Example B-280: N-{[1-(cyclopentylmethyl)-5-fluoro-6-[2-(trifluoromethyl)phenyl]-1H-indol-3-yl]methyl}cyclopropanesulfonamide

### Step 1. 6-bromo-1-(cyclopentylmethyl)-5-fluoro-1H-indole-3-carbaldehyde

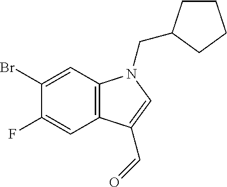 [SMILES: O=Cc1cn(CC2CCCC2)c2cc(Br)c(F)cc12]

To a solution of 6-bromo-5-fluoro-1H-indole-3-carbaldehyde (1 g, 4.13 mmol) and bromomethylcyclopentane (1.01 g, 6.20 mmol) in DMF (10 mL), were added Cs₂CO₃ (2.69 g, 8.26 mmol) and potassium iodide (68.58 mg, 413.15 μmol). The mixture was stirred at 80° C. for 1 hr and cooled to room temperature. Water (100 mL) was added. The mixture was extracted with EA (2×50 mL). The combined organic layer was washed with brine (50 mL), dried over anhydrous Na₂SO₄, filtered and concentrated in vacuo to give crude product which was purified by silica gel column chromatography to give the product as a colorless oil (1.3 g, yield: 97%).

### Step 2. (E)-6-bromo-1-(cyclopentylmethyl)-5-fluoro-1H-indole-3-carbaldehyde O-methyl oxime

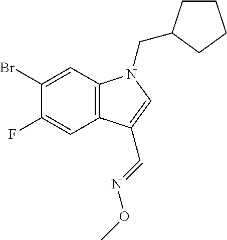 [SMILES: CO/N=C/c1cn(CC2CCCC2)c2cc(Br)c(F)cc12]

To a suspension of 6-bromo-1-(cyclopentylmethyl)-5-fluoro-1H-indole-3-carbaldehyde (496.91 mg, 1.53 mmol) and O-methylhydroxylamine hydrochloride (288.51 mg, 6.13 mmol) in H₂O (2 mL) and EtOH (10 mL), was added Na₂CO₃ (324.92 mg, 3.07 mmol). The mixture solution was refluxed for 2 hr. The resulting reaction was concentrated under reduced pressure, and then water (100 mL) was added. The mixture was extracted with EA (2×100 mL). The combined organic layer was washed with brine (2×50 mL), dried over anhydrous Na₂SO₄, filtered, and concentrated under reduced pressure. The residue was purified by silica gel column to give the product as a yellow solid (530 mg, yield: 97%).

### Step 3. [6-bromo-1-(cyclopentylmethyl)-5-fluoro-indol-3-yl]methanamine

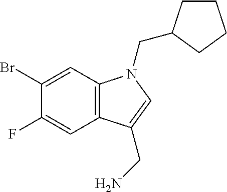 [SMILES: NCc1cn(CC2CCCC2)c2cc(Br)c(F)cc12]

The solution of (E)-6-bromo-1-(cyclopentylmethyl)-5-fluoro-1H-indole-3-carbaldehyde O-methyl oxime (576 mg, 1.63 mmol) and borane-THF (1 N in THF, 7 mL) was refluxed for 1 hr and cooled to room temperature. Conc. HCl (2 mL) was added. The reaction mixture was stirred for another 10 min. The solvent was removed under reduced pressure. The residue was purified by prep-HPLC to the product as a white solid (393 mg, yield: 74%).

### Step 4. N-((6-bromo-1-(cyclopentylmethyl)-5-fluoro-1H-indol-3-yl)methyl)cyclopropanesulfonamide

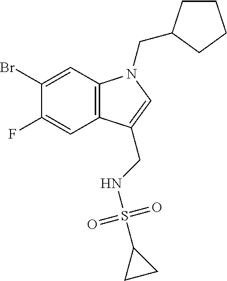 [SMILES: O=S(=O)(NCc1cn(CC2CCCC2)c2cc(Br)c(F)cc12)C1CC1]

To a solution of [6-bromo-1-(cyclopentylmethyl)-5-fluoro-indol-3-yl]methanamine (393 mg, 1.21 mmol) and cyclopropanesulfonyl chloride (339.78 mg, 2.42 mmol) in DCM (10 mL) in an ice-water bath, was added TEA (489.12 mg, 4.83 mmol). The mixture was allowed to warm to room temperature and stirred for 2 hr. The solvent was removed under reduced pressure. The residue was purified by prep-HPLC to give the product as a yellow solid (400 mg, yield: 77%).

### Step 5. N-{[1-(cyclopentylmethyl)-5-fluoro-6-[2-(trifluoromethyl)phenyl]-1H-indol-3-yl]methyl}cyclopropanesulfonamide

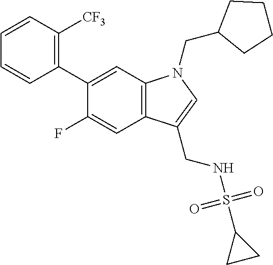 [SMILES: O=S(=O)(NCc1cn(CC2CCCC2)c2cc(-c3ccccc3C(F)(F)F)c(F)cc12)C1CC1]

To a solution of compound N-((6-bromo-1-(cyclopentylmethyl)-5-fluoro-1H-indol-3-yl)methyl)cyclopropanesulfonamide (100 mg, 232.91 μmol), K₃PO₄·7H₂O (236.36 mg, 699.29 μmol) and [2-(trifluoromethyl)phenyl]boronic acid (66.04 mg, 347.74 μmol) in dioxane (50 mL), was added Pd(dppf)Cl₂ (15 mg) under N₂ atmosphere. The reaction mixture was stirred at 85° C. for 3 h. The resulting reaction was concentrated under reduced pressure, and then water (30 mL) was added. The mixture was extracted with EA (2×20 mL). The combined organic layers were washed with brine (2×50 mL), dried over Na₂SO₄, filtered, and concentrated under reduced pressure. The residue was purified by prep-HPLC to give the product as a yellow solid (66 mg, yield: 57%). ¹H NMR (400 MHz, d₆-DMSO): δ 7.86 (d, J=7.6 Hz, 1H), 7.74 (t, J=7.2 Hz, 1H), 7.65 (t, J=7.6 Hz, 1H), 7.41-7.51 (m, 5H), 4.32 (d, J=6.0 Hz, 2H), 3.99-4.10 (m, 2H), 2.43-2.52 (m, 1H), 2.28-2.33 (m, 1H), 1.45-1.59 (m, 6H), 1.19-1.25 (m, 2H), 0.83-0.92 (m, 4H). LCMS (Method A) RT 2.537 min, calcd. for C₂₅H₂₆F₄N₂O₂S 494.17 m/z, found 511.1 m/z [M+H]⁺.

### Example B-281: N-{2-[6-(3-chloropyridin-4-yl)-1-(cyclopentylmethyl)-1H-indol-3-yl]ethyl}cyclopropanesulfonamide

### Step 1. 6-bromo-1-(cyclopentylmethyl)-1H-indole-3-carbaldehyde

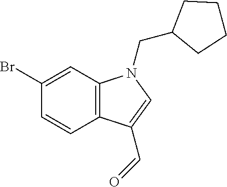 [SMILES: O=Cc1cn(CC2CCCC2)c2cc(Br)ccc12]

To a stirred solution of 6-bromo-1H-indole-3-carbaldehyde (500 mg, 2.24 mmol) in DMF (10 mL) were added (bromomethyl)cyclopentane (548 mg, 3.36 mmol) and K₂CO₃ (464 mg, 3.36 mmol). The resulting reaction mixture was stirred at 70° C. for 1 h and cooled down to room temperature at which time water (50 mL) and EA (50 mL) were added. The organic layer was separated and the aqueous layer was extracted with EA (2×50 mL). The combined organic layer was washed with brine (2×50 mL), dried over anhydrous Na₂SO₄ and evaporated to give the desired product (580 mg, yield: 84.81%) as yellow oil.

### Step 2. (E)-6-bromo-1-(cyclopentylmethyl)-3-(2-nitrovinyl)-1H-indole

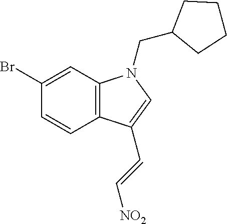 [SMILES: O=[N+]([O-])/C=C/c1cn(CC2CCCC2)c2cc(Br)ccc12]

A mixture of 6-bromo-1-(cyclopentylmethyl)-1H-indole-3-carbaldehyde (580 mg, 1.90 mmol) and ammonium acetate (900 mg, 11.67 mmol) in nitromethane (5 mL) was stirred at 80° C. for 1 hr. The solvent was then removed and the residue was partitioned between EA (50 mL) and water (50 mL). The organic layer was separated and the aqueous layer was extracted with EA (2×50 mL). The combined organic layer was washed with brine (3×50 mL), dried over anhydrous Na₂SO₄ and evaporated to give the desired product (510 mg, yield: 77.07%) as yellow oil.

### Step 3. 6-bromo-1-(cyclopentylmethyl)-3-(2-nitroethyl)-1H-indole

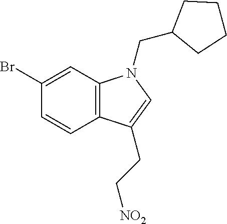 [SMILES: O=[N+]([O-])CCc1cn(CC2CCCC2)c2cc(Br)ccc12]

To a solution of (E)-6-bromo-1-(cyclopentylmethyl)-3-(2-nitrovinyl)-1H-indole (510 mg, 1.47 mmol) in MeOH (10 mL), was added NaBH₄ (300 mg, 7.94 mmol) in portions at 0° C. The mixture was stirred for 1 h at that temperature, poured into ice-water and the solvent was removed. The residue was extracted with EA (2×50 mL), the combined organic layer was washed with brine (2×50 mL), dried and evaporated to give crude product which was used to the next step directly.

### Step 4. 2-(6-bromo-1-(cyclopentylmethyl)-1H-indol-3-yl)ethanamine

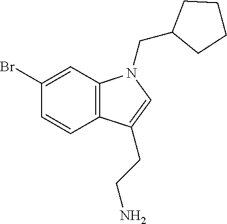 [SMILES: NCCc1cn(CC2CCCC2)c2cc(Br)ccc12]

To a solution of 6-bromo-1-(cyclopentylmethyl)-3-(2-nitroethyl)-1H-indole and HCl (12 N, 5 mL) in MeOH (8 mL), was added Fe (750 mg, 13.43 mmol). The mixture was refluxed for 2 hr. The solid was filtered off and washed with MeOH. The combined organic layer was concentrated and purified by pre-HPLC to give the desired product (150 mg, yield: 31.99%) as yellow oil.

### Step 5. N-(2-(6-bromo-1-(cyclopentylmethyl)-1H-indol-3 yl)ethyl)cyclopropanesulfonamide

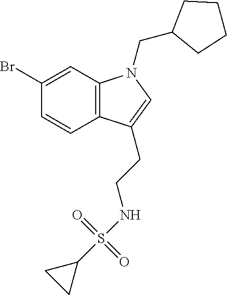 [SMILES: O=S(=O)(NCCc1cn(CC2CCCC2)c2cc(Br)ccc12)C1CC1]

To a solution of 2-(6-bromo-1-(cyclopentylmethyl)-1H-indol-3-yl)ethanamine (150 mg, 0.35 mmol) and DIEA (226 mg, 1.75 mmol) in DCM (3 mL), was added cyclopropanesulfonyl chloride (100 mg, 0.70 mmol) dropwise at −20° C. The mixture was stirred for 1 h at that −20° C. at which time the solvent was removed. The residue was purified by pre-HPLC to give the desired product (150 mg, yield: 75.47%) as a white solid.

### Step 6. N-(2-(6-(3-chloropyridin-4-yl)-1-(cyclopentylmethyl)-1H-indol-3-yl)ethyl)cyclopropanesulfonamid

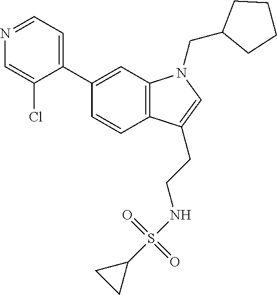 [SMILES: O=S(=O)(NCCc1cn(CC2CCCC2)c2cc(-c3ccncc3Cl)ccc12)C1CC1]

To a suspension of N-(2-(6-bromo-1-(cyclopentylmethyl)-1H-indol-3 yl)ethyl)cyclopropanesulfonamide (150 mg, 0.35 mol), 3-chloro-4-(4,4,5,5-tetramethyl-1,3,2-dioxaborolan-2-yl)pyridine (150 mg, 731.49 μmol) and K₃PO₄·7H₂O (250 mg, 1.18 mmol) in dioxane (3 mL), was added Pd(dppf)Cl₂ (50 mg) under N₂ atmosphere. The reaction mixture was reacted at 85° C. for 3 h and cooled down to room temperature. The resulting reaction was poured in to water (50 mL) and extracted with EA (2×25 mL). The combined organic layer was washed with brine (25 mL), dried over Na₂SO₄, filtered and concentrated. The residue was purified by pre-HPLC to give the desired product (63 mg, yield: 38.97%) as a white solid. ¹HNMR (400 MHz, d₆-DMSO) δ ¹H NMR (400 MHz, d₆-DMSO) δ 8.72 (s, 1H), 8.57 (d, J=5.2 Hz, 1H), 7.62-7.67 (m, 2H), 7.53 (d, J=5.2 Hz, 1H), 7.39 (s, 1H), 7.14-7.24 (m, 2H), 4.08 (d, J=7.6 Hz, 2H), 3.22-3.35 (m, 2H), 2.93 (t, J=7.6 Hz, 2H), 2.50-2.55 (m, 1H), 2.30-2.45 (m, 1H), 1.45-1.65 (m, 6H), 1.21-1.52 (m, 2H), 0.87-0.93 (m, 4H). LCMS (Method A) RT 1.90 min, calcd. for C₂₄H₂₈ClN₃O₂S 457.16 m/z, found 458.4 m/z [M+H]⁺.

### Example B-282: N-{2-[6-(3-chloropyridin-4-yl)-1-(cyclopentylmethyl)-5-fluoro-1H-indol-3-yl]ethyl}cyclopropanesulfonamide

### Step 1. 6-bromo-1-(cyclopentylmethyl)-5-fluoro-3-[(E)-2-nitrovinyl]indole

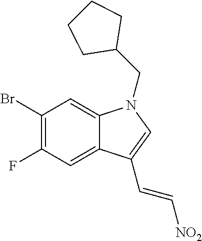 [SMILES: O=[N+]([O-])/C=C/c1cn(CC2CCCC2)c2cc(Br)c(F)cc12]

To a solution of 6-bromo-1-(cyclopentylmethyl)-5-fluoro-1H-indole-3-carbaldehyde (496.91 mg, 1.53 mmol) in nitromethane (10 mL), was added AcONH₄ (235.6 mg, 3.06 mmol). The mixture was stirred at 70° C. for 1 hr. The resulting reaction was concentrated under reduced pressure, and water (30 mL) was added. The mixture was extracted with EA (2×30 mL). The combined organic layer was washed with brine (2×40 mL), dried over Na₂SO₄, filtered, and concentrated to give the product as a yellow solid (557 mg, yield: 98%).

### Step 2. 6-bromo-1-(cyclopentylmethyl)-5-fluoro-3-(2-nitroethyl)-1H-indole

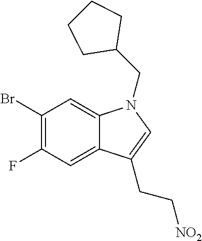 [SMILES: O=[N+]([O-])CCc1cn(CC2CCCC2)c2cc(Br)c(F)cc12]

To a solution of 6-bromo-1-(cyclopentylmethyl)-5-fluoro-3-[(E)-2-nitrovinyl]indole (553.96 mg, 1.51 mmol) in MeOH (20 mL), was added NaBH₄ (57.07 mg, 1.51 mmol) in portions at 0° C. The mixture was stirred for 1 h at that temperature, and poured into ice-water. The solvent was removed under reduced pressure. The residue was extracted with EA (2×30 mL). The combined organic layer was washed with brine (2×30 mL), dried and evaporated to give crude product which was used to the next step directly.

### Step 3. 2-(6-bromo-1-(cyclopentylmethyl)-5-fluoro-1H-indol-3-yl)ethanamine

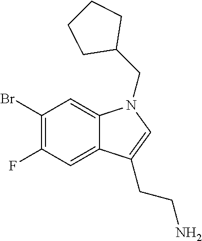 [SMILES: NCCc1cn(CC2CCCC2)c2cc(Br)c(F)cc12]

To a solution of 6-bromo-1-(cyclopentylmethyl)-5-fluoro-3-(2-nitroethyl)-1H-indole (554 mg, 5.93 mmol) and HCl (12 N, 2 mL) in MeOH (4 mL), was added Fe (252.76 mg, 4.53 mmol). The mixture was refluxed for 2 hr. The solid was filtered off and washed with MeOH. The combined organic layer was concentrated and purified by prep-HPLC to give the product as a white solid (100 mg, yield: 19%).

### Step 4. N-[2-[6-bromo-1-(cyclopentylmethyl)-5-fluoro-indol-3-yl]ethyl]cyclopropanesulfonamide

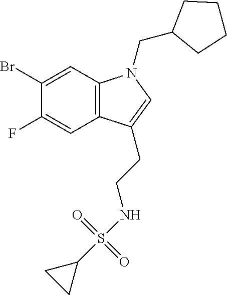 [SMILES: O=S(=O)(NCCc1cn(CC2CCCC2)c2cc(Br)c(F)cc12)C1CC1]

To a solution of 2-(6-bromo-1-(cyclopentylmethyl)-5-fluoro-1H-indol-3-yl)ethanamine (330 mg, 0.98 mmol) and DIEA (378 mg, 2.93 mmol) in DCM (10 mL), was added cyclopropanesulfonyl chloride (206 mg, 1.47 mmol) dropwise at 0° C. The mixture was allowed to warm to room temperature and stirred for 1 h. The solvent was removed under reduced pressure. The residue was purified by prep-HPLC to give the product as a yellow solid (280 mg, yield: 67%).

### Step 5. N-{2-[6-(3-chloropyridin-4-yl)-1-(cyclopentylmethyl)-5-fluoro-1H-indol-3-yl]ethyl}cyclopropanesulfonamide

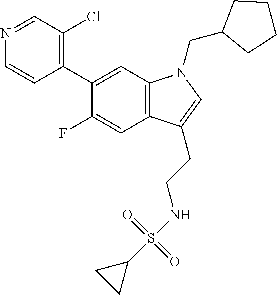 [SMILES: O=S(=O)(NCCc1cn(CC2CCCC2)c2cc(-c3ccncc3Cl)c(F)cc12)C1CC1]

To a suspension of N-[2-[6-bromo-1-(cyclopentylmethyl)-5-fluoro-indol-3-yl]ethyl]cyclopropanesulfonamide (130 mg, 293.21 μmol), 3-chloro-4-(4,4,5,5-tetramethyl-1,3,2-dioxaborolan-2-yl)pyridine (105.34 mg, 439.81 μmol) and K₃PO₄·7H₂O (212.94 mg, 0.63 mmol) in dioxane (5 mL), was added Pd(dppf)Cl₂ (20 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (40 mg, yield: 28%). ¹H NMR (400 MHz, d₆-DMSO): δ 8.76 (d, J=7.6 Hz, 1H), 8.61 (d, J=5.2 Hz, 1H), 7.53-7.57 (m, 2H), 7.44 (t, J=8 Hz, 2H), 7.18 (t, J=5.6 Hz, 1H), 4.06 (d, J=7.2 Hz, 2H), 3.23-3.28 (m, 2H), 2.90 (t, J=7.6 Hz, 2H), 2.49-2.56 (m, 1H) 2.31-2.38 (m, 1H), 1.47-1.64 (m, 6H), 1.19-1.26 (m, 2H), 0.85-0.91 (m, 4H). LCMS (Method A), RT 2.316 min, calcd. for C₂₄H₂₇ClFN₃O₂S 475.15 m/z, found 476.2m/z [M+H]⁺.

### Example B-283: N-{[6-(3-chloropyridin-4-yl)-5-fluoro-1-(2-methylpropyl)-1H-indol-3-yl]methyl}-1,1,1-trifluoromethanesulfonamide

### Step 1. N-((6-bromo-5-fluoro-1-isobutyl-1H-indol-3-yl)methyl)-1,1,1-trifluoromethanesulfonamide

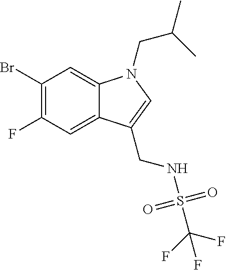 [SMILES: CC(C)Cn1cc(CNS(=O)(=O)C(F)(F)F)c2cc(F)c(Br)cc21]

To a solution of (6-bromo-5-fluoro-1-isobutyl-1H-indol-3-yl)methanamine (300 mg, 1.00 mmol) and TEA (152.20 mg, 1.50 mmol) in EA (10 mL), was added trifluoromethanesulfonyl chloride (152.09 mg, 902.46 μmol) dropwise at −40° C. The mixture was allowed to warm to room temperature and stirred for 30 min. Water (30 mL) was added. The organic layer was separated. The aqueous layer was extracted with EA (2×30 mL). The combined organic layer was washed with brine (2×30 mL), dried over Na₂SO₄, filtered, and concentrated under reduced pressure. The residue was purified by prep-HPLC to give the product as a yellow solid (400 mg, yield: 92%).

### Step 2. N-{[6-(3-chloropyridin-4-yl)-5-fluoro-1-(2-methylpropyl)-1H-indol-3-yl]methyl}-1,1,1-trifluoromethanesulfonamide

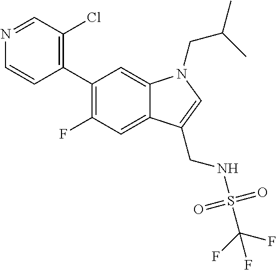 [SMILES: CC(C)Cn1cc(CNS(=O)(=O)C(F)(F)F)c2cc(F)c(-c3ccncc3Cl)cc21]

To a suspension of N-((6-bromo-5-fluoro-1-isobutyl-1H-indol-3-yl)methyl)-1,1,1-trifluoromethanesulfonamide (100 mg, 232.91 μmol), 3-chloro-4-(4,4,5,5-tetramethyl-1,3,2-dioxaborolan-2-yl)pyridine (83.31 mg, 347.83 μmol) and K₃PO₄·7H₂O (157.51 mg 465.82 μmol) in dioxane (5 mL), was added Pd(dppf)Cl₂ (44 mg) under N₂ atmosphere. The reaction mixture was stirred at 85° C. for 3 h and cooled to room temperature. The solvent was removed under reduced pressure. The residue was purified by prep-HPLC to give the product as a yellow solid (100 mg, yield: 61%). ¹H NMR (400 MHz, d₆-DMSO): δ 9.76 (s, 1H), 8.77 (s, 1H), 8.62 (d, J=4.8 Hz, 1H), 7.63 (d, J=5.6 Hz, 1H), 7.52-7.56 (m, 3H), 4.52 (s, 2H), 4.00 (d, J=7.2 Hz, 2H), 2.03-2.13 (m, 1H), 0.84 (m, J=6.4 Hz, 6H). LCMS (Method A) RT 2.297 min, calcd. for C₁₉H₁₈ClF₄N₃O₂S 463.07 m/z, found 464.2 m/z [M+H]⁺.

### Example B-284: N-{[6-(3-chloropyridin-4-yl)-5-fluoro-1-[3,3,3-trifluoro-2-(trifluoromethyl)propyl]-1H-indol-3-yl]methyl}cyclopropanesulfonamide

### Step 1. (E)-6-bromo-5-fluoro-1-(3,3,3-trifluoro-2-(trifluoromethyl)propyl)-1H-indole-3-carbaldehyde O-methyl oxime

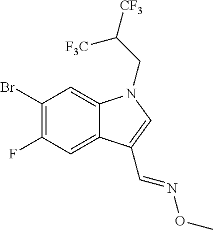 [SMILES: CO/N=C/c1cn(CC(C(F)(F)F)C(F)(F)F)c2cc(Br)c(F)cc12]

To a solution of 6-bromo-5-fluoro-1-(3,3,3-trifluoro-2-(trifluoromethyl)propyl)-1H-indole-3-carbaldehyde (496.91 mg, 1.53 mmol) and O-methylhydroxylamine hydrochloride (508.79 mg, 6.13 mmol) in H₂O (2 mL) and EtOH (10 mL), was added AcONa (502.66 mg, 6.13 mmol). The mixture was stirred at room temperature overnight. The resulting reaction was concentrated under reduced pressure, and then water (30 mL) was added. The mixture was extracted with EA (2×30 mL). The combined organic layers were washed with brine (2×50 mL), dried over Na₂SO₄, filtered, and concentrated to give the product as a yellow solid (530 mg, yield: 97%).

### Step 2. (6-bromo-5-fluoro-1-(3,3,3-trifluoro-2-(trifluoromethyl)propyl)-1H-indol-3-yl)methanamine

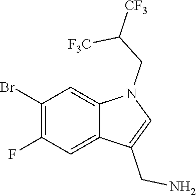 [SMILES: NCc1cn(CC(C(F)(F)F)C(F)(F)F)c2cc(Br)c(F)cc12]

A mixture of (E)-6-bromo-5-fluoro-1-(3,3,3-trifluoro-2-(trifluoromethyl)propyl)-1H-indole-3-carbaldehyde O-methyl oxime (576 mg, 1.63 mmol) and borane-THF (1 N in THF, 7 mL) was refluxed for 1 hr and cooled to room temperature. Conc. HCl (12 N, 2 mL) was added. The mixture was stirred for 10 min, and then the solvent was removed under reduced pressure. The residue was purified by prep-HPLC to give the product as a white solid (300 mg, yield: 74%).

### Step 3. N-((6-bromo-5-fluoro-1-(3,3,3-trifluoro-2-(trifluoromethyl)propyl)-1H-indol-3-yl)methyl)cyclopropanesulfonamide

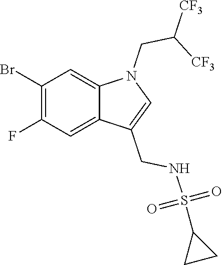 [SMILES: O=S(=O)(NCc1cn(CC(C(F)(F)F)C(F)(F)F)c2cc(Br)c(F)cc12)C1CC1]

To a solution of (6-bromo-5-fluoro-1-(3,3,3-trifluoro-2-(trifluoromethyl)propyl)-1H-indol-3-yl)methanamine (300 mg, 736.88 μmol) and DIEA (190.47 mg, 1.47 mmol) in DCM (10 mL) in an ice-water bath, was added cyclopropanesulfonyl chloride (155.40 mg, 1.11 mmol) dropwise. The mixture was allowed to warm to room temperature and stirred overnight. The solvent was removed under reduced pressure. The residue was purified by prep-HPLC to give the product as a yellow solid (300 mg, yield: 79%).

### Step 4. N-{[6-(3-chloropyridin-4-yl)-5-fluoro-1-[3,3,3-trifluoro-2-(trifluoromethyl)propyl]-1H-indol-3-yl]methyl}cyclopropanesulfonamide

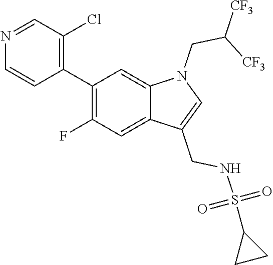 [SMILES: O=S(=O)(NCc1cn(CC(C(F)(F)F)C(F)(F)F)c2cc(-c3ccncc3Cl)c(F)cc12)C1CC1]

To a solution of compound N-((6-bromo-5-fluoro-1-(3,3,3-trifluoro-2-(trifluoromethyl)propyl)-1H-indol-3-yl)methyl)cyclopropanesulfonamide (100 mg, 195.60 μmol), K₃PO₄·7H₂O (66.11 mg, 195.60 μmol) and 3-chloro-4-(4,4,5,5-tetramethyl-1,3,2-dioxaborolan-2-yl)pyridine (46.85 mg, 195.60 μmol) in dioxane (10 mL), was added Pd(dppf)Cl₂ (20 mg) under N₂ atmosphere. The reaction mixture was stirred at 85° C. for 3 h and cooled to room temperature. The resulting reaction was concentrated under reduced pressure, and then water (30 mL) was added. The mixture was extracted with EA (2×30 mL). The combined organic layer was washed with brine (2×30 mL), dried over Na₂SO₄, filtered, and concentrated under reduced pressure. The residue was purified by prep-HPLC to give the product as a yellow solid (55 mg, yield: 51%). ¹H NMR (400 MHz, d₆-DMSO): δ 8.75 (s, 1H), 8.63 (d, J=4.8 Hz, 1H), 7.52-7.63 (m, 5H), 4.75-4.82 (m, 3H), 4.30 (d, J=6.0 Hz, 2H), 2.39-2.5 (m, 1H), 0.81-0.91 (m, 4H). LCMS (Method A) RT 2.181 min, calcd. for C₂₁H₁₇ClF₇N₃O₂S 543.06 m/z, found 544.1 m/z [M+H]⁺.

### Example B-285: N-{[6-(3-chloropyridin-4-yl)-1-(2,2-dimethylpropanoyl)-5-fluoro-1H-indol-3-yl]methyl}-1,1,1-trifluoromethanesulfonamide

### Step 1. (E)-6-bromo-5-fluoro-1H-indole-3-carbaldehyde O-methyl oxime

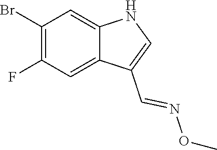

To a suspension of 6-bromo-5-fluoro-1H-indole-3-carbaldehyde (1.00 g, 4.13 mmol) and O-methylhydroxylamine hydrochloride (500 mg, 5.99 mmol) in H₂O (1 mL) and EtOH (5 mL), was added AcONa (500 mg, 6.10 mmol). The mixture was stirred for 12 h. The solvent was removed under reduced pressure. The residue was partitioned between EA (50 mL) and water (50 mL). The organic layer was separated. The aqueous layer was extracted with EA (2×50 mL). The combined organic layers were washed with brine (2×50 mL), dried over anhydrous Na₂SO₄ and evaporated to give the product as a yellow oil (1.00 g, 89% yield).

### Step 2. (6-bromo-5-fluoro-1H-indol-3-yl)methanamine

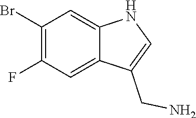 [SMILES: NCc1c[nH]c2cc(Br)c(F)cc12]

A mixture of (E)-6-bromo-5-fluoro-1H-indole-3-carbaldehyde O-methyl oxime (1.00 g, 3.69 mmol) and borane-THF (1 N in THF, 8 mL) was refluxed for 1 hr and cooled to room temperature. HCl (12 N, 1 mL) was added. The reaction mixture was stirred for another 10 min. The solvent was removed under reduced pressure. The residue was purified by prep-HPLC to give the product as a brown oil (380 mg, 42% yield).

### Step 3. tert-butyl (6-bromo-5-fluoro-1H-indol-3-yl)methylcarbamate

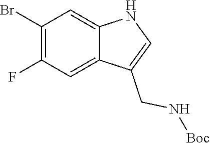 [SMILES: CC(C)(C)OC(=O)NCc1c[nH]c2cc(Br)c(F)cc12]

To a solution of compound (6-bromo-5-fluoro-1H-indol-3-yl)methanamine (380 mg, 1.56 mmol) and TEA (217.80 mg, 2.15 mmol) in DCM (5.0 mL) in an ice-water bath, was added (Boc)₂O (380 mg, 1.74 mmol) dropwise. The reaction mixture was allowed to warm to room temperature and stirred for 0.5 h. The solvent was removed under reduced pressure. The residue was purified by prep-HPLC to give the product as a yellow oil (450 mg, yield: 83%).

### Step 4. tert-butyl (6-bromo-5-fluoro-1-pivaloyl-1H-indol-3-yl)methylcarbamate

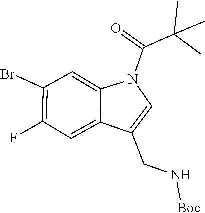 [SMILES: CC(C)(C)OC(=O)NCc1cn(C(=O)C(C)(C)C)c2cc(Br)c(F)cc12]

To a solution of tert-butyl (6-bromo-5-fluoro-1H-indol-3-yl)methylcarbamate (450 mg, 1.31 mmol), DMAP (15 mg, 122.78 μmol) and TEA (363 mg, 3.59 mmol) in DCM (3 mL), was added pivaloyl chloride (450 mg, 3.40 mmol) dropwise at 0° C. The reaction mixture was allowed to warm to room temperature and stirred for 2 h. The solvent was removed under reduced pressure. The residue was purified by prep-HPLC to give the product as a yellow oil (300 mg, yield: 52%).

### Step 5. 1-(3-(aminomethyl)-6-bromo-5-fluoro-1H-indol-1-yl)-2,2-dimethylpropan-1-one

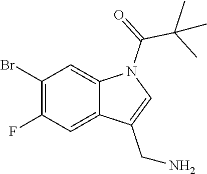 [SMILES: CC(C)(C)C(=O)n1cc(CN)c2cc(F)c(Br)cc21]

To a solution of tert-butyl (6-bromo-5-fluoro-1-pivaloyl-1H-indol-3-yl)methylcarbamate (300 mg, 683.06 μmol) in DCM (6 mL) in an ice-water bath, was added TFA (3 mL) dropwise. The reaction mixture was allowed to warm to room temperature and stirred for 1 h. The solvent was removed under reduced pressure. The residue was purified by prep-HPLC to give the product as a yellow solid (210 mg, yield: 91%).

### Step 6. N-((6-bromo-5-fluoro-1-pivaloyl-1H-indol-3-yl)methyl)-1,1,1-trifluoromethanesulfonamide

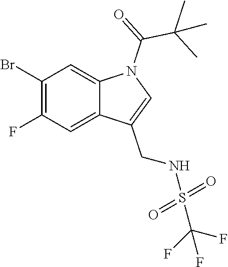 [SMILES: CC(C)(C)C(=O)n1cc(CNS(=O)(=O)C(F)(F)F)c2cc(F)c(Br)cc21]

To a solution of 1-(3-(aminomethyl)-6-bromo-5-fluoro-1H-indol-1-yl)-2,2-dimethylpropan-1-one (210 mg, 463.47 μmol) and TEA (145 mg, 1.43 mmol) in EA (5 mL), was added trifluoromethanesulfonyl chloride (200 mg, 1.19 mmol) dropwise at −78° C. The reaction mixture was allowed to warm to room temperature and stirred for 2 h. The solvent was removed under reduced pressure. The residue was purified by prep-HPLC to give the product as a white solid (150 mg, yield: 68%).

### Step 7. N-{[6-(3-chloropyridin-4-yl)-1-(2,2-dimethylpropanoyl)-5-fluoro-1H-indol-3-yl]methyl}-1,1,1-trifluoromethanesulfonamide

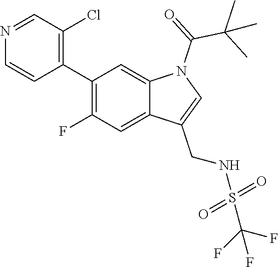 [SMILES: CC(C)(C)C(=O)n1cc(CNS(=O)(=O)C(F)(F)F)c2cc(F)c(-c3ccncc3Cl)cc21]

To a suspension of N-((6-bromo-5-fluoro-1-pivaloyl-1H-indol-3-yl)methyl)-1,1,1-trifluoromethanesulfonamide (150 mg, 318.37 μmol), 3-chloro-4-(4,4,5,5-tetramethyl-1,3,2-dioxaborolan-2-yl)pyridine (150 mg, 626.29 μmol) and K₃PO₄·7H₂O (200 mg, 942.21 μmol) in dioxane (3 mL), was added Pd(dppf)Cl₂ (20 mg, 27.33 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a yellow solid (51 mg, yield: 31%). ¹HNMR (400 MHz, d₆-DMSO) δ 9.96 (s, 1H), 8.77 (s, 1H), 8.62 (d, J=4.8 Hz, 1H), 8.37 (d, J=6.4 Hz, 1H), 8.30 (s, 1H), 7.65 (d, J=10.0 Hz, 1H), 7.54 (d, J=4.8 Hz, 1H), 4.61 (s, 2H), 1.44 (s, 9H). LCMS (Method B), RT 2.127 min, calcd. for C₂₀H₁₈ClF₄N₃O₃S 491.07 m/z, found 492.0 m/z [M+H]⁺.

### Example B-286: N-{[6-(3-chloropyridin-4-yl)-5-fluoro-1-{[1-(trifluoromethyl)cyclopropyl]methyl}-1H-indol-3-yl]methyl}cyclopropanesulfonamide

### Step 1. (1-(trifluoromethyl)cyclopropyl)methanol

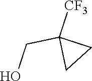

A mixture of 1-(trifluoromethyl)cyclopropane-1-carboxylic acid (0.50 g, 3.25 mmol) BH₃ (1 N in THF, 5 mL) was refluxed for 1 hr and cooled to room temperature. 1 mL 12 N aqueous HCl was added. The reaction mixture was stirred for another 10 min. The solvent was removed under reduced pressure. The residue was partitioned between Et₂O (20 mL) and water (20 mL). The organic layer was separated. The aqueous layer was extracted with Et₂O (2×20 mL). The combined organic layer was washed with brine (2×50 mL), dried over anhydrous Na₂SO₄ and evaporated to give the product as a colorless oil (0.35 g, yield: 77%).

### Step 2. (1-(trifluoromethyl)cyclopropyl)methyl 4-methylbenzenesulfonate

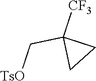 [SMILES: Cc1ccc(S(=O)(=O)OCC2(C(F)(F)F)CC2)cc1]

To a solution of (1-(trifluoromethyl)cyclopropyl)methanol (350 mg, 2.5 mmol) and TEA (757.5 mg, 7.5 mmol) in DCM (5 mL) in an ice-water bath, was added TsCl (573 mg, 3.0 mmol) in portions. The mixture was allowed to warm to room temperature and stirred for 2 h. The solvent was removed under reduced pressure. The residue was purified by prep-HPLC to give the product as a colorless oil (0.52 g, yield: 70%).

### Step 3. 6-bromo-5-fluoro-1-((1-(trifluoromethyl)cyclopropyl)methyl)-1H-indole-3-carbaldehyde

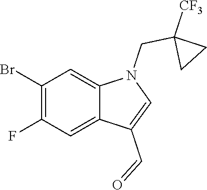 [SMILES: O=Cc1cn(CC2(C(F)(F)F)CC2)c2cc(Br)c(F)cc12]

To a solution of 6-bromo-5-fluoro-1H-indole-3-carbaldehyde (400 mg, 1.66 mmol) and (1-(trifluoromethyl)cyclopropyl)methyl 4-methylbenzenesulfonate (586 mg, 1.99 mmol) in DMF (10 mL), were added Cs₂CO₃ (1.08 g, 3.32 mmol) and KI (0.1 g). The mixture was stirred at 80° C. for 1 h and cooled to room temperature. Water (20 mL) and EA (20 mL) was added. The organic layer was separated. The aqueous layer was extracted with EA (2×30 mL). The combined organic layer was washed with brine (2×30 mL), dried over anhydrous Na₂SO₄ and evaporated to give crude product which was purified by silica gel column chromatography to give the product as a yellow oil (550 mg, yield: 91%).

### Step 4. (E)-6-bromo-5-fluoro-1-((1-(trifluoromethyl)cyclopropyl)methyl)-1H-indole-3-carbaldehyde O-methyl oxime

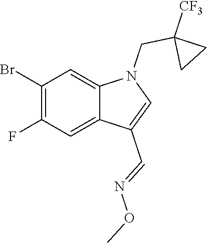 [SMILES: CO/N=C/c1cn(CC2(C(F)(F)F)CC2)c2cc(Br)c(F)cc12]

To a suspension of 6-bromo-5-fluoro-1-((1-(trifluoromethyl)cyclopropyl)methyl)-1H-indole-3-carbaldehyde (300 mg, 0.83 mmol) and O-methylhydroxylamine hydrochloride (137 mg, 1.65 mmol) in H₂O (1 mL) and EtOH (5 mL), was added AcONa (135.3 mg, 1.65 mmol). The mixture was stirred at room temperature overnight. The solvent was removed under reduced pressure. The residue was partitioned between EA (20 mL) and water (20 mL). The organic layer was separated. The aqueous layer was extracted with EA (2×20 mL). The combined organic layers were washed with brine (2×20 mL), dried over anhydrous Na₂SO₄ and evaporated to give the product as a yellow solid (280 mg, 86% yield).

### Step 5. (6-bromo-5-fluoro-1-((1-(trifluoromethyl)cyclopropyl)methyl)-1H-indol-3-yl)methanamine

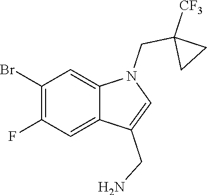 [SMILES: NCc1cn(CC2(C(F)(F)F)CC2)c2cc(Br)c(F)cc12]

A mixture of (E)-6-bromo-5-fluoro-1-((1-(trifluoromethyl)cyclopropyl)methyl)-1H-indole-3-carbaldehyde O-methyl oxime (280 mg, 0.74 mmol) and borane-THF (1 N in THF, 3 mL) was refluxed for 1 hr and cooled to room temperature. 1 mL 12 N aqueous HCl was added. The reaction mixture was stirred for another 10 min. The solvent was removed under reduced pressure. The residue was purified by prep-HPLC to give the product as a yellow oil (210 mg, yield: 81%).

### Step 6. N-((6-bromo-5-fluoro-1-((1-(trifluoromethyl)cyclopropyl)methyl)-1H-indol-3-yl)methyl)cyclopropanesulfonamide

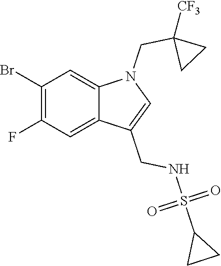 [SMILES: O=S(=O)(NCc1cn(CC2(C(F)(F)F)CC2)c2cc(Br)c(F)cc12)C1CC1]

To a mixture of (6-bromo-5-fluoro-1-((1-(trifluoromethyl)cyclopropyl)methyl)-1H-indol-3-yl)methanamine (210 mg, 0.58 mmol) and TEA (175 mg, 1.73 mmol) in DCM (5 mL), was added cyclopropanesulfonyl chloride (121.8 mg, 0.87 mmol) dropwise at room temperature. The mixture was stirred for 2 h. The solvent was removed under reduced pressure. The residue was purified by prep-HPLC to give the product as a white solid (180 mg, yield: 67%).

### Step 7. N-{[6-(3-chloropyridin-4-yl)-5-fluoro-1-{[1-(trifluoromethyl)cyclopropyl]methyl}-1H-indol-3-yl]methyl}cyclopropanesulfonamide

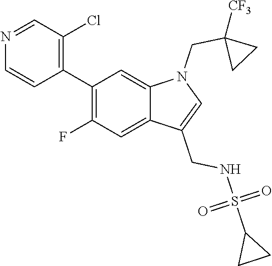 [SMILES: O=S(=O)(NCc1cn(CC2(C(F)(F)F)CC2)c2cc(-c3ccncc3Cl)c(F)cc12)C1CC1]

To a suspension of N-((6-bromo-5-fluoro-1-((1-(trifluoromethyl)cyclopropyl)methyl)-1H-indol-3-yl)methyl)cyclopropanesulfonamide (180 mg, 0.38 mmol), 3-chloro-4-(4,4,5,5-tetramethyl-1,3,2-dioxaborolan-2-yl)pyridine (138 mg, 0.58 mmol) and K₃PO₄·7H₂O (372 mg, 1.14 mmol) in dioxane (5 mL), was added Pd(dppf)Cl₂ (15 mg) under N₂ atmosphere. The reaction mixture was reacted at 85° C. for 3 h and cooled to room temperature. The resulting reaction mixture was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (42.7 mg, yield: 22%). ¹H NMR (400 MHz, d₆-DMSO): δ 8.76 (s, 1H), 8.61 (d, J=4.8 Hz, 1H), 7.48-7.60 (m, 5H), 4.49 (s, 2H), 4.31 (d, J=6.0 Hz, 2H), 2.42-2.45 (m, 1H), 1.01 (s, 4H), 0.87-0.91 (m, 2H), 0.81-0.96 (m, 2H). LCMS (Method A) RT 2.153 min, calcd. for C₂₂H₂₀ClF₄N₃O₂S 510.09 m/z, found 502.2 m/z [M+H]⁺.

### Example B-287: N-{2-[1-(2,2-dimethylpropyl)-5-fluoro-6-[2-(trifluoromethyl)phenyl]-1H-indol-3-yl]ethyl}cyclopropanesulfonamide

### Step 1. (E)-6-bromo-5-fluoro-1-neopentyl-3-(2-nitrovinyl)-1H-indole

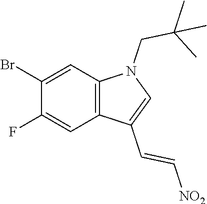 [SMILES: CC(C)(C)Cn1cc(/C=C/[N+](=O)[O-])c2cc(F)c(Br)cc21]

To a solution of 6-bromo-5-fluoro-1-neopentyl-1H-indole-3-carbaldehyde (2.0 g, 6.41 mmol) in nitromethane (9 mL), was added AcONH₄ (2.5 g, 32.43 mmol). The mixture was stirred at 70° C. for 1 hr. The resulting reaction was concentrated under reduced pressure, and water (30 mL) was added. The mixture was extracted with EA (2×30 mL). The combined organic layer was washed with brine (2×40 mL), dried over Na₂SO₄, filtered, and concentrated to give the product as a yellow oil (2.1 g, yield: 92%).

### Step 2. 6-bromo-5-fluoro-1-neopentyl-3-(2-nitroethyl)-1H-indole

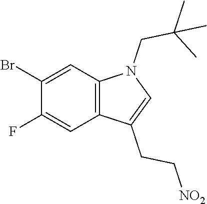 [SMILES: CC(C)(C)Cn1cc(CC[N+](=O)[O-])c2cc(F)c(Br)cc21]

To a solution of (E)-6-bromo-5-fluoro-1-neopentyl-3-(2-nitrovinyl)-1H-indole (2.1 g, 5.92 mmol) in MeOH (12 mL), was added NaBH₄ (1.36 g, 36.04 mmol) in portions at 0° C. The mixture was stirred for 2 h at that temperature, and poured into ice-water. The solvent was removed under reduced pressure. The residue was extracted with EA (2×50 mL). The combined organic layer was washed with brine (2×50 mL), dried and evaporated to give crude product which was used to the next step directly.

### Step 3. 2-(6-bromo-5-fluoro-1-neopentyl-1H-indol-3-yl)ethanamine

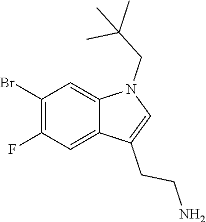 [SMILES: CC(C)(C)Cn1cc(CCN)c2cc(F)c(Br)cc21]

To a solution of 6-bromo-5-fluoro-1-neopentyl-3-(2-nitroethyl)-1H-indole (2.1 g, 5.88 mmol) and HCl (12 N, 3 mL) in MeOH (10 mL), was added Fe (1.84 g, 32.95 mmol). The mixture was refluxed for 2 hr. The solid was filtered off and washed with MeOH. The combined organic layer was concentrated and purified by prep-HPLC to give the product as a yellow solid (600 mg, yield: 32%).

### Step 4. N-(2-(6-bromo-5-fluoro-1-neopentyl-1H-indol-3-yl)ethyl)cyclopropanesulfonamide

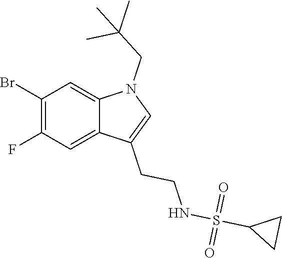 [SMILES: CC(C)(C)Cn1cc(CCNS(=O)(=O)C2CC2)c2cc(F)c(Br)cc21]

To a solution of 2-(6-bromo-5-fluoro-1-neopentyl-1H-indol-3-yl)ethanamine (300 mg, 0.92 mmol) and DIEA (400 mg, 3.10 mmol) in DCM (3 mL), was added cyclopropanesulfonyl chloride (300 mg, 2.13 mmol) dropwise at −20° C. The mixture was allowed to warm to room temperature and stirred for 2 h. The solvent was removed under reduced pressure. The residue was purified by prep-HPLC to give the product as a white solid (300 mg, yield: 75%).

### Step 5. N-{2-[1-(2,2-dimethylpropyl)-5-fluoro-6-[2-(trifluoromethyl)phenyl]-1H-indol-3-yl]ethyl}cyclopropanesulfonamide

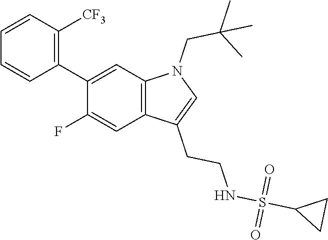 [SMILES: CC(C)(C)Cn1cc(CCNS(=O)(=O)C2CC2)c2cc(F)c(-c3ccccc3C(F)(F)F)cc21]

To a suspension of N-(2-(6-bromo-5-fluoro-1-neopentyl-1H-indol-3-yl)ethyl)cyclopropanesulfonamide (200 mg, 464 μmol), [2-(trifluoromethyl)phenyl]boronic acid (200 mg, 1.05 mmol) and K₃PO₄·7H₂O (300 mg, 1.41 mmol) in dioxane (3 mL), was added Pd(dppf)Cl₂ (50 mg, 68.33 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (117 mg, yield: 50%). ¹HNMR (400 MHz, d₆-DMSO) δ 7.85 (d, J=8.0 Hz, 1H), 7.60-7.78 (m, 2H), 7.39-7.46 (m, 2H), 7.36 (d, J=10.4 Hz, 1H), 7.30 (s 1H), 7.20 (t, J=6.0 Hz, 1H), 4.00 (d, J=14.0 Hz, 1H), 3.85 (d, J=14.0 Hz, 1H), 3.23-3.31 (m, 2H), 2.90 (t, J=7.6 Hz, 2H), 2.52-2.55 (m, 1H), 0.85-0.92 (m, 13H). LCMS (Method B), RT 2.562 min, calcd. for C₂₅H₂₈F₄N₂O₂S 496.18 m/z, found 497.2 m/z [M+H]⁺.

### Example B-288: N-{2-[6-(3-chloropyridin-4-yl)-1-(2,2-dimethylpropyl)-5-fluoro-1H-indol-3-yl]ethyl}cyclopropanesulfonamide

### Step 1. N-{2-[6-(3-chloropyridin-4-yl)-1-(2,2-dimethylpropyl)-5-fluoro-1H-indol-3-yl]ethyl}cyclopropanesulfonamide

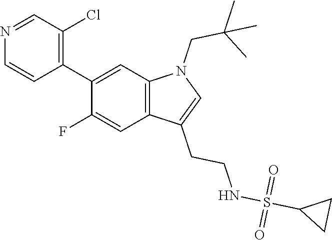 [SMILES: CC(C)(C)Cn1cc(CCNS(=O)(=O)C2CC2)c2cc(F)c(-c3ccncc3Cl)cc21]

To a suspension of N-(2-(6-bromo-5-fluoro-1-neopentyl-1H-indol-3-yl)ethyl)cyclopropanesulfonamide (200 mg, 464 μmol), (3-chloro-4-pyridyl)boronic acid (150 mg, 953.21 μmol) and K₃PO₄·7H₂O (300 mg, 1.41 mmol) in dioxane (3 mL), was added Pd(dppf)Cl₂ (50 mg, 68.33 μmol) under N₂ atmosphere. The reaction mixture was reacted at 85° C. for 3 h. The resulting reaction was cooled to room temperature, poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (64 mg, yield: 29%). ¹HNMR (400 MHz, d₆-DMSO) δ 8.76 (s, 1H), 8.61 (d, J=5.2 Hz, 1H), 7.59 (d, J=6.0 Hz, 1H), 7.50 (d, J=4.8 Hz, 1H), 7.45 (d, J=11.2 Hz, 1H), 7.35 (s, 1H), 7.15-7.23 (m, 1H), 3.97 (s, 2H), 3.22-3.30 (m, 2H), 2.91 (t, J=7.6 Hz, 2H), 2.52-2.57 (m, 1H), 0.86-0.94 (m, 13H). LCMS (Method B), RT 2.290 min, calcd. for C₂₃H₂₇ClFN₃O₂S 463.15 m/z, found 464.2 m/z [M+H]⁺.

### Example B-289: N-{2-[1-(2,2-dimethylpropyl)-5-fluoro-6-[2-(trifluoromethyl)phenyl]-1H-indol-3-yl]ethyl}propane-2-sulfonamide

### Step 1. N-(2-(6-bromo-5-fluoro-1-neopentyl-1H-indol-3-yl)ethyl)propane-2-sulfonamide

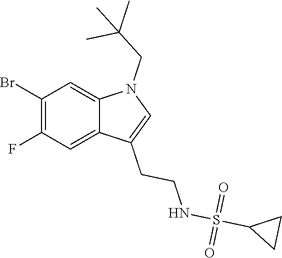 [SMILES: CC(C)(C)Cn1cc(CCNS(=O)(=O)C2CC2)c2cc(F)c(Br)cc21]

To a mixture of 2-(6-bromo-5-fluoro-1-neopentyl-1H-indol-3-yl)ethanamine (300 mg, 0.92 mmol) and TEA (278.83 mg, 2.76 mmol) in DCM (5 mL), was added propane-2-sulfonyl chloride (156.77 mg, 1.10 mmol) at −40° C. The mixture was allowed to warm to room temperature and stirred for 2 h. The solvent was removed under reduced pressure. The residue was purified by prep-HPLC to give the product as a yellow oil (180 mg, yield: 45%).

### Step 2. N-{2-[1-(2,2-dimethylpropyl)-5-fluoro-6-[2-(trifluoromethyl)phenyl]-1H-indol-3-yl]ethyl}propane-2-sulfonamide

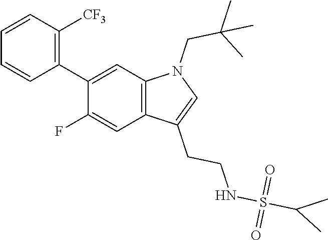 [SMILES: CC(C)S(=O)(=O)NCCc1cn(CC(C)(C)C)c2cc(-c3ccccc3C(F)(F)F)c(F)cc12]

To a suspension of N-(2-(6-bromo-5-fluoro-1-neopentyl-1H-indol-3-yl)ethyl)propane-2-sulfonamide (90 mg, 0.21 mmol), 2-(trifluoromethyl)phenylboronic acid (47.50 mg, 0.25 mmol) and K₃PO₄·7H₂O (212.94 mg, 0.63 mmol) in dioxane (5 mL), was added Pd(dppf)Cl₂ (20 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (42 mg, yield: 40%). ¹H NMR (400 MHz, d₆-DMSO): δ 7.85 (d, J=7.6 Hz, 1H), 7.74 (t, J=7.2 Hz, 1H), 7.65 (t, J=7.6 Hz, 1H), 7.41-7.44 (m, 2H), 7.36 (d, J=10.4 Hz, 1H), 7.29 (s, 1H), 7.13 (t, J=5.6 Hz, 1H), 3.99 (d, J=14.0 Hz, 1H), 3.84 (d, J=14.4 Hz, 1H), 3.21-3.26 (m, 2H), 3.08-3.15 (m, 1H), 2.87 (t, J=7.6 Hz, 2H), 1.16 (d J=6.8 Hz, 6H), 0.90 (s, 9H). LCMS (Method A) RT 2.578 min, calcd. for C₂₅H₃₀F₄N₂O₂S 498.20 m/z, found 499.2 m/z [M+H]⁺.

### Example B-290: N-{2-[6-(3-chloropyridin-4-yl)-1-(2,2-dimethylpropyl)-5-fluoro-1H-indol-3-yl]ethyl}propane-2-sulfonamide

### Step 1. N-{2-[6-(3-chloropyridin-4-yl)-1-(2,2-dimethylpropyl)-5-fluoro-1H-indol-3-yl]ethyl}propane-2-sulfonamide

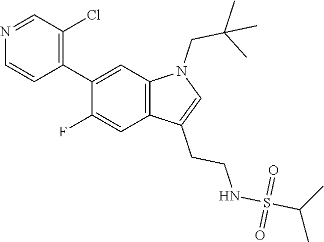 [SMILES: CC(C)S(=O)(=O)NCCc1cn(CC(C)(C)C)c2cc(-c3ccncc3Cl)c(F)cc12]

To a suspension of N-(2-(6-bromo-5-fluoro-1-neopentyl-1H-indol-3-yl)ethyl)propane-2-sulfonamide (90 mg, 0.21 mmol), 3-chloro-4-(4,4,5,5-tetramethyl-1,3,2-dioxaborolan-2-yl)pyridine (59.75 mg, 0.25 mmol) and K₃PO₄·7H₂O (212.94 mg, 0.63 mmol) in dioxane (5 mL), was added Pd(dppf)Cl₂ (20 mg) under N₂ atmosphere. The reaction mixture was reacted at 85° C. for 3 h and cooled to room temperature. The resulting reaction was poured into water (50 mL) and extracted with EA (2×25 mL). The combined organic layer was washed with brine (25 mL), dried over Na₂SO₄, filtered, and concentrated under reduced pressure. The residue was purified by prep-HPLC to give the product as a white solid (43.64 mg, yield: 44%).

¹H NMR (400 MHz, d₆-DMSO): δ 8.76 (s, 1H), 8.61 (d, J=4.8 Hz, 1H), 7.59 (d, J=6.0 Hz, 1H), 7.49 (d, J=5.2 Hz, 1H), 7.44 (d, J=10.8 Hz, 1H), 7.35 (s, 1H), 7.13 (t, J=5.6 Hz, 1H), 3.96 (s, 2H), 3.20-3.25 (m, 2H), 3.10-3.19 (m, 1H), 2.88 (t, J=7.2 Hz, 2H), 1.17 (d, J=6.8 Hz, 6H), 0.93 (s, 9H). LCMS (Method A) RT 2.338 min, calcd. for C₂₃H₂₉ClFN₃O₂S 465.17 m/z, found 466.2 m/z [M+H]⁺.

Compound Synthesis Examples: Part 3. Compound numbers in Part 3 are shown in parentheses in each example.

### Example 366: (S)-2-methyl-N-(2,2,2-trifluoro-1-(5-fluoro-1-neopentyl-6-(2-(trifluoromethyl)phenyl)-1H-indol-3-yl)ethyl)propane-2-sulfonamide (366)

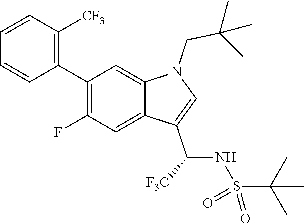 [SMILES: CC(C)(C)Cn1cc([C@H](NS(=O)(=O)C(C)(C)C)C(F)(F)F)c2cc(F)c(-c3ccccc3C(F)(F)F)cc21]

To a solution of 2-methyl-N—((S)-2,2,2-trifluoro-1-(5-fluoro-1-neopentyl-6-(2-(trifluoromethyl)phenyl)-1H-indol-3-yl)ethyl)propane-2-sulfinamide (0.0756 g, 0.137 mmol) in DCM (1 mL) was added mCPBA (0.036 g, 0.205 mmol). The reaction was monitored by anal. HPLC for completion. The mixture was diluted with DCM, washed with saturated NaHCO₃ and brine. The solvent was removed and the crude was purified by prep-HPLC to obtain the desired title compd. ESI-MS (m/z): 566.78 [M+1]⁺.

### Example 367: (S)—N-(2,2,2-trifluoro-1-(6-(4-fluoro-2-(trifluoromethyl)phenyl)-1-neopentyl-1H-indol-3-yl)ethyl)cyclopropanesulfonamide (367)

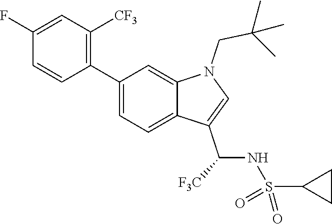 [SMILES: CC(C)(C)Cn1cc([C@H](NS(=O)(=O)C2CC2)C(F)(F)F)c2ccc(-c3ccc(F)cc3C(F)(F)F)cc21]

### Step 1: 6-(4-fluoro-2-(trifluoromethyl)phenyl)-1-neopentyl-1H-indole

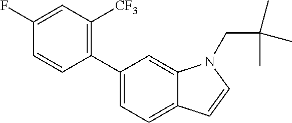 [SMILES: CC(C)(C)Cn1ccc2ccc(-c3ccc(F)cc3C(F)(F)F)cc21]

The title compound was prepared following the same general protocol as described for Step 5, Example 1, using 6-bromo-1-neopentyl-1H-indole and (4-fluoro-2-(trifluoromethyl)phenyl)boronic acid. ESI-MS (m/z): 349.85 [M+1]⁺.

### Step 2: (R)-2-methyl-N—((S)-2,2,2-trifluoro-1-(6-(4-fluoro-2-(trifluoromethyl)phenyl)-1-neopentyl-1H-indol-3-yl)ethyl)propane-2-sulfinamide

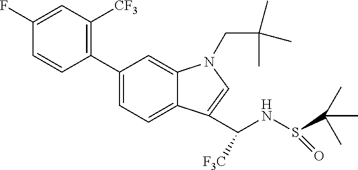 [SMILES: CC(C)(C)Cn1cc([C@H](N[S@](=O)C(C)(C)C)C(F)(F)F)c2ccc(-c3ccc(F)cc3C(F)(F)F)cc21]

The title compound was prepared following the same general protocol as described for Step 3, Example 116, using (S)-2-methyl-N-(3,3,3-trifluoroprop-1-en-1-yl)propane-2-sulfinamide and 6-(4-fluoro-2-(trifluoromethyl)phenyl)-1-neopentyl-1H-indole. ESI-MS (m/z): 551.70 [M+1]⁺.

### Step 3: (S)-2,2,2-trifluoro-1-(6-(4-fluoro-2-(trifluoromethyl)phenyl)-1-neopentyl-1H-indol-3-yl)ethan-1-amine

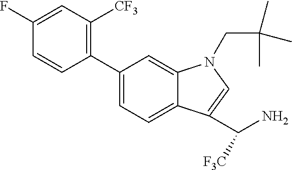 [SMILES: CC(C)(C)Cn1cc([C@H](N)C(F)(F)F)c2ccc(-c3ccc(F)cc3C(F)(F)F)cc21]

The title compound was prepared following the same general protocol as described for Step 4, Example 116, using 2-methyl-N—((S)-2,2,2-trifluoro-1-(6-(4-fluoro-2-(trifluoromethyl)phenyl)-1-neopentyl-1H-indol-3-yl)ethyl)propane-2-sulfinamide.

### Step 4: (S)—N-(2,2,2-trifluoro-1-(6-(4-fluoro-2-(trifluoromethyl)phenyl)-1-neopentyl-1H-indol-3-yl)ethyl)cyclopropanesulfonamide

The title compound was prepared following the same general protocol as described for Step 5, Example 116, using (S)-2,2,2-trifluoro-1-(6-(4-fluoro-2-(trifluoromethyl)phenyl)-1-neopentyl-1H-indol-3-yl)ethan-1-amine. HNMR (CDCl₃, 400 MHz) δ 7.75 (d, J=8.0 Hz, 1H), 7.48 (dd, J=8.0 Hz, 4.0 Hz, 1H), 7.39-7.36 (m, 1H), 7.29-7.24 (m, 3H), 7.12 (d, J=8.0 Hz, 1H), 5.45 (q, J=8.0 Hz, 1H), 4.82 (d, J=8.0 Hz, 1H), 3.89 (s, 2H), 2.48-2.42 (m, 1H), 1.25-1.21 (m, 2H), 0.99 (s, 9H), 0.98-0.96 (m, 2H)

### Example 368: (S)—N-(1-(6-bromo-7-methyl-1-neopentyl-1H-indol-3-yl)-2,2,2-trifluoroethyl)cyclopropanesulfonamide (368)

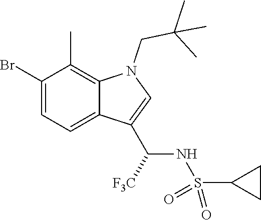 [SMILES: Cc1c(Br)ccc2c([C@H](NS(=O)(=O)C3CC3)C(F)(F)F)cn(CC(C)(C)C)c12]

### Step 1: 6-bromo-7-methyl-1-neopentyl-1H-indole

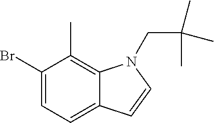 [SMILES: Cc1c(Br)ccc2ccn(CC(C)(C)C)c12]

The title compound was prepared following the same general protocol as described for Example 34, using 6-bromo-7-methyl-1H-indole and 1-iodo-2,2-dimethylpropane. ESI-MS (m/z): 279.80, 281.80 [M+1]⁺.

### Step 2: (S)—N—((S)-1-(6-bromo-7-methyl-1-neopentyl-1H-indol-3-yl)-2,2,2-trifluoroethyl)-2-methylpropane-2-sulfinamide

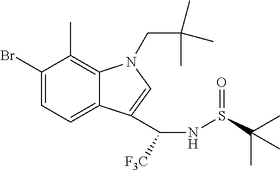 [SMILES: Cc1c(Br)ccc2c([C@H](N[S@@](=O)C(C)(C)C)C(F)(F)F)cn(CC(C)(C)C)c12]

The title compound was prepared following the same general protocol as described for Step 3, Example 116, using (S)-2-methyl-N-(3,3,3-trifluoroprop-1-en-1-yl)propane-2-sulfinamide and 6-bromo-7-methyl-1-neopentyl-1H-indole. ESI-MS (m/z): 482.51 [M+1]⁺.

### Step 3: (S)-1-(6-bromo-7-methyl-1-neopentyl-1H-indol-3-yl)-2,2,2-trifluoroethan-1-amine

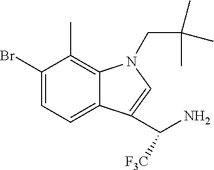 [SMILES: Cc1c(Br)ccc2c([C@H](N)C(F)(F)F)cn(CC(C)(C)C)c12]

The title compound was prepared following the same general protocol as described for Step 4, Example 116, using (S)—N—((S)-1-(6-bromo-7-methyl-1-neopentyl-1H-indol-3-yl)-2,2,2-trifluoroethyl)-2-methylpropane-2-sulfinamide.

### Step 4: (S)—N-(1-(6-bromo-7-methyl-1-neopentyl-1H-indol-3-yl)-2,2,2-trifluoroethyl)cyclopropanesulfonamide

The title compound was prepared following the same general protocol as described for Step 5, Example 116, using: (S)-1-(6-bromo-7-methyl-1-neopentyl-1H-indol-3-yl)-2,2,2-trifluoroethan-1-amine. ESI-MS (m/z): 482.08 [M+1]⁺.

### Example 369: (S)—N-(1-(6-bromo-7-fluoro-1-neopentyl-1H-indol-3-yl)-2,2,2-trifluoroethyl)cyclopropanesulfonamide (369)

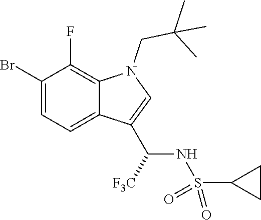 [SMILES: CC(C)(C)Cn1cc([C@H](NS(=O)(=O)C2CC2)C(F)(F)F)c2ccc(Br)c(F)c21]

### Step 1: 6-bromo-7-fluoro-1-neopentyl-1H-indole

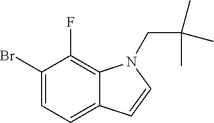 [SMILES: CC(C)(C)Cn1ccc2ccc(Br)c(F)c21]

The title compound was prepared following the same general protocol as described for Example 34, using 6-bromo-7-fluoro-1H-indole and 1-iodo-2,2-dimethylpropane. ESI-MS (m/z): 283.77, 285.79 [M+1]⁺.

### Step 2: (S)—N—((S)-1-(6-bromo-7-fluoro-1-neopentyl-1H-indol-3-yl)-2,2,2-trifluoroethyl)-2-methylpropane-2-sulfinamide

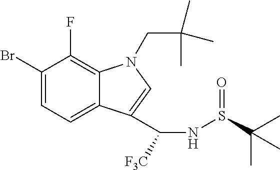 [SMILES: CC(C)(C)Cn1cc([C@H](N[S@@](=O)C(C)(C)C)C(F)(F)F)c2ccc(Br)c(F)c21]

The title compound was prepared following the same general protocol as described for Step 3, Example 116, using (S)-2-methyl-N-(3,3,3-trifluoroprop-1-en-1-yl)propane-2-sulfinamide and 6-bromo-7-fluoro-1-neopentyl-1H-indole. ESI-MS (m/z): 484.55, 486.54 [M+1]⁺.

### Step 3: (S)-1-(6-bromo-7-fluoro-1-neopentyl-1H-indol-3-yl)-2,2,2-trifluoroethan-1-amine

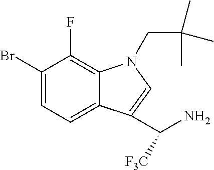 [SMILES: CC(C)(C)Cn1cc([C@H](N)C(F)(F)F)c2ccc(Br)c(F)c21]

The title compound was prepared following the same general protocol as described for Step 4, Example 116, using (S)—N—((S)-1-(6-bromo-7-fluoro-1-neopentyl-1H-indol-3-yl)-2,2,2-trifluoroethyl)-2-methylpropane-2-sulfinamide.

### Step 4: (S)—N-(1-(6-bromo-7-fluoro-1-neopentyl-1H-indol-3-yl)-2,2,2-trifluoroethyl)cyclopropanesulfonamide

The title compound was prepared following the same general protocol as described for Step 5, Example 116, using: (S)-1-(6-bromo-7-fluoro-1-neopentyl-1H-indol-3-yl)-2,2,2-trifluoroethan-1-amine. ESI-MS (m/z): 485.74, 486.64 [M+1]⁺.

### Example 370: (S)—N-(2,2,2-trifluoro-1-(1-isobutyl-6-(1-methyl-1H-pyrazol-5-yl)-1H-indol-3-yl)ethyl)cyclopropanesulfonamide (370)

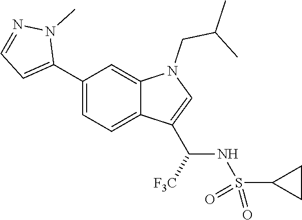 [SMILES: CC(C)Cn1cc([C@H](NS(=O)(=O)C2CC2)C(F)(F)F)c2ccc(-c3ccnn3C)cc21]

The title compound was prepared following the same general protocol as described for Step 5, Example 1, using N—((S)-1-(6-bromo-1-isobutyl-1H-indol-3-yl)-2,2,2-trifluoroethyl)cyclopropanesulfinamide and 1-methyl-5-(4,4,5,5-tetramethyl-1,3,2-dioxaborolan-2-yl)-1H-pyrazole. ESI-MS (m/z): 455.01 [M+1]⁺.

### Example 371: (S)-methyl-N-(2,2,2-trifluoro-1-(5-fluoro-1-neopentyl-6-(2-(trifluoromethyl)phenyl)-1H-indol-3-yl)ethyl)sulfamoyl)amine (371)

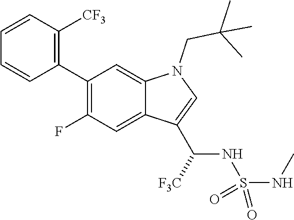 [SMILES: CNS(=O)(=O)N[C@@H](c1cn(CC(C)(C)C)c2cc(-c3ccccc3C(F)(F)F)c(F)cc12)C(F)(F)F]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and methylsulfamoyl chloride. ¹HNMR (CDCl₃, 400 MHz) δ 7.79 (d, J=8.0 Hz, 1H), 7.59 (t, J=8.0 Hz, 1H), 7.52 (t, J=8.0 Hz, 1H), 7.45 (d, J=8.0 Hz, 1H), 7.37 (d, J=8.0 Hz, 1H), 7.27 (s, 1H), 7.22 (d, J=8.0 Hz, 1H), 5.22 (q, J=8.0 Hz, 1H), 4.95 (d, J=8.0 Hz, 1H), 4.29-4.25 (m, 1H), 3.91-3.80 (m, 2H), 2.67 (broad S, 3H), 0.97 (s, 9H)

### Example 372: (S)—N-(2,2,2-trifluoro-1-(5-fluoro-1-neopentyl-6-(2-(trifluoromethyl)phenyl)-1H-indol-3-yl)ethyl)cyclopentanesulfonamide (372)

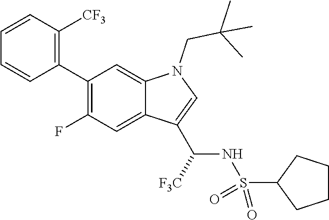 [SMILES: CC(C)(C)Cn1cc([C@H](NS(=O)(=O)C2CCCC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and cyclopentanesulfonyl chloride. ESI-MS (m/z): 579.21 [M+1]⁺.

### Example 373: (S)—N-(2,2,2-trifluoro-1-(6-(2-fluoro-6-(trifluoromethyl)phenyl)-1-neopentyl-1H-indol-3-yl)ethyl)cyclopropanesulfonamide (373)

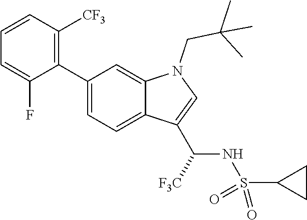 [SMILES: CC(C)(C)Cn1cc([C@H](NS(=O)(=O)C2CC2)C(F)(F)F)c2ccc(-c3c(F)cccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and cyclopropanesulfonyl chloride. ¹HNMR (CDCl₃, 400 MHz) δ 7.78 (d, J=8.0 Hz, 1H), 7.59 (t, J=8.0 Hz, 1H), 7.52 (t, J=8.0 Hz, 1H), 7.35 (d, J=8.0 Hz, 1H), 7.28 (d, J=8.0 Hz, 1H), 7.27 (s, 1H), 7.21 (d, J=8.0 Hz, 1H), 5.30 (q, J=8.0 Hz, 1H), 5.12-4.95 (m, 1H), 4.29-4.25 (m, 1H), 3.98-3.74 (m, 3H), 2.53-41 (m, 2H), 2.31-2.09 (m, 2H), 1.96-1.84 (m, 2H), 0.96 (s, 9H)

### Example 374: (S)—N-(2,2,2-trifluoro-1-(5-fluoro-1-neopentyl-6-(2-(trifluoromethyl)phenyl)-1H-indol-3-yl)ethyl)cyclobutanesulfonamide (374)

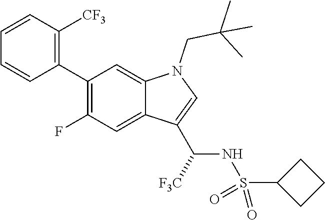 [SMILES: CC(C)(C)Cn1cc([C@H](NS(=O)(=O)C2CCC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and cyclobutanesulfonyl chloride. ¹HNMR (CDCl₃, 400 MHz) δ 7.78 (d, J=8.0 Hz, 1H), 7.48 (dd, J=8.0 Hz, 4.0 Hz, 1H), 7.39-7.36 (m, 1H), 7.29-7.24 (m, 3H), 7.12 (d, J=8.0 Hz, 1H), 5.45 (q, J=8.0 Hz, 1H), 4.82 (d, J=8.0 Hz, 1H), 3.89 (s, 2H), 2.48-2.42 (m, 1H), 1.25-1.21 (m, 2H), 0.99 (s, 9H), 0.98-0.96 (m, 2H). LCMS: 564.8 m/z [M+1]⁺.

### Example 375: (S)-1-methyl-N-(2,2,2-trifluoro-1-(5-fluoro-1-neopentyl-6-(2-(trifluoromethyl)phenyl)-1H-indol-3-yl)ethyl)cyclopropane-1-sulfonamide (375)

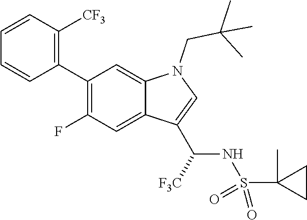 [SMILES: CC(C)(C)Cn1cc([C@H](NS(=O)(=O)C2(C)CC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and 1-methylcyclopropane-1-sulfonyl chloride. ¹HNMR (CDCl₃, 400 MHz) δ 7.78 (d, J=8.0 Hz, 1H), 7.59 (t, J=8.0 Hz, 1H), 7.52 (t, J=8.0 Hz, 1H), 7.38-7.33 (m, 1H), 7.32 (d, J=8.0 Hz, 1H), 7.27 (s, 1H), 7.21 (d, J=8.0 Hz, 1H), 5.20 (q, J=8.0 Hz, 1H), 5.01-4.86 (m, 1H), 3.93-3.82 (m, 2H), 1.88-1.74 (m, 1H), 1.44 (s, 3H), 1.27-1.10 (m, 1H), 0.96 (s, 9H), 0.88-0.68 (m, 2H)

### Example 376: (S)—N-(1-(6-bromo-1-neopentyl-5-(trifluoromethyl)-1H-indol-3-yl)-2,2,2-trifluoroethyl)cyclopropanesulfonamide (376)

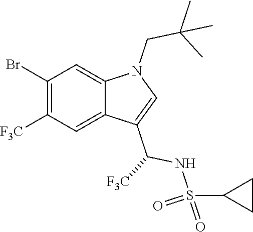 [SMILES: CC(C)(C)Cn1cc([C@H](NS(=O)(=O)C2CC2)C(F)(F)F)c2cc(C(F)(F)F)c(Br)cc21]

### Step 1: 6-bromo-1-neopentyl-5-(trifluoromethyl)-1H-indole

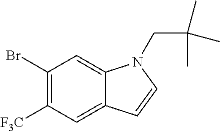 [SMILES: CC(C)(C)Cn1ccc2cc(C(F)(F)F)c(Br)cc21]

The title compound was prepared following the same general protocol as described for Example 34, using 6-bromo-5-(trifluoromethyl)-1H-indole and 1-iodo-2,2-dimethylpropane. ¹HNMR (CDCl₃, 400 MHz) δ 7.91 (s, 1H), 7.41 (s, 1H), 7.17 (d, J=4.0 Hz, 1H), 6.59 (d, J=4.0 Hz, 1H), 3.94 (s, 2H), 1.01 (s, 9H)

### Step 2: (S)—N—((S)-1-(6-bromo-1-neopentyl-5-(trifluoromethyl)-1H-indol-3-yl)-2,2,2-trifluoroethyl)-2-methylpropane-2-sulfinamide

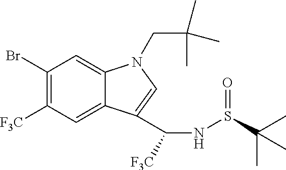 [SMILES: CC(C)(C)Cn1cc([C@H](N[S@@](=O)C(C)(C)C)C(F)(F)F)c2cc(C(F)(F)F)c(Br)cc21]

The title compound was prepared following the same general protocol as described for Step 3, Example 116, using (S)-2-methyl-N-(3,3,3-trifluoroprop-1-en-1-yl)propane-2-sulfinamide and 6-bromo-1-neopentyl-5-(trifluoromethyl)-1H-indole. ESI-MS (m/z): 535.01, 536.55 [M+1]⁺.

### Step 3: (S)-1-(6-bromo-7-fluoro-1-neopentyl-1H-indol-3-yl)-2,2,2-trifluoroethan-1-amine

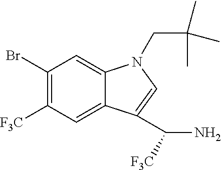 [SMILES: CC(C)(C)Cn1cc([C@H](N)C(F)(F)F)c2cc(C(F)(F)F)c(Br)cc21]

The title compound was prepared following the same general protocol as described for Step 4, Example 116, using (S)—N—((S)-1-(6-bromo-1-neopentyl-5-(trifluoromethyl)-1H-indol-3-yl)-2,2,2-trifluoroethyl)-2-methylpropane-2-sulfinamide.

### Step 4: (S)—N-(1-(6-bromo-1-neopentyl-5-(trifluoromethyl)-1H-indol-3-yl)-2,2,2-trifluoroethyl)cyclopropanesulfonamide

The title compound was prepared following the same general protocol as described for Step 5, Example 116, using (S)-1-(6-bromo-7-fluoro-1-neopentyl-1H-indol-3-yl)-2,2,2-trifluoroethan-1-amine. ¹HNMR (CDCl₃, 400 MHz) δ 8.10 (s, 1H), 7.68 (s, 1H), 7.25 (s, 1H), 5.42-5.35 (q, J=8.0 Hz, 1H), 5.05 (d, J=8.0 Hz, 1H), 3.83 (s, 2H), 2.48-2.42 (m, 1H), 1.25-1.21 (m, 2H), 0.99 (s, 9H), 0.98-0.96 (m, 2H)

### Example 377: (S)—N-(2,2,2-trifluoro-1-(5-fluoro-1-isobutyl-6-(1H-pyrazol-5-yl)-1H-indol-3-yl)ethyl)cyclopropanesulfonamide (377)

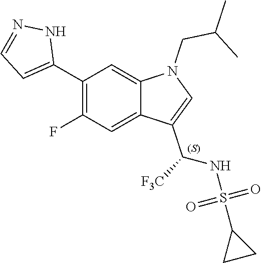

A mixture of N-[(1S)-1-(6-bromo-5-fluoro-1-isobutyl-indol-3-yl)-2,2,2-trifluoro-ethyl]cyclopropanesulfonamide (60 mg, 127.31 μmol, 1H-pyrazol-5-ylboronic acid (25.25 mg, 225.62 umol), Pd(dppf)Cl2 (368.78 mg, 0.504 mmol) and Na₂CO₃ (40.48 mg, 381.93 μmol) in 1,4-dioxane (3 mL) and water (0.7 mL) was stirred at 80° C. overnight under Nitrogen. the mixture was concentrated, to the residue was added water, the aqueous phase was extracted with EtOAc twice. the combined organic phases were were washed with brine, dried with anhydrous Na₂SO₄ and filtered. The filtrate was concentrated to get oil, which was purified by prep-HPLC to get N-[(1S)-2,2,2-trifluoro-1-[5-fluoro-1-isobutyl-6-(1H-pyrazol-5-yl)indol-3-yl]ethyl]cyclopropanesulfonamide (36.5 mg, 79.61 μmol) as a white solid. 1H NMR (400 MHz, DMSO) δ 13.10 (d, J=111.1 Hz, 1H), 8.57 (s, 1H), 7.95 (d, J=6.1 Hz, 1H), 7.81 (d, J=13.3 Hz, 1H), 7.70 (dd, J=42.2, 28.0 Hz, 2H), 6.64 (s, 1H), 5.45 (s, 1H), 4.03 (d, J=7.1 Hz, 2H), 2.35-2.24 (m, 1H), 2.12 (dd, J=13.3, 6.6 Hz, 1H), 1.20-0.38 (m, 10H). LCMS: 458.8 m/z [M+H]⁺.

### Example 378: N-[(1S)-1-[1-(2,2-dimethylpropyl)-5-fluoro-6-(5,6,7,8-tetrahydroquinolin-4-yl)indol-3-yl]-2,2,2-trifluoro-ethyl]cyclopropanesulfonamide (378)

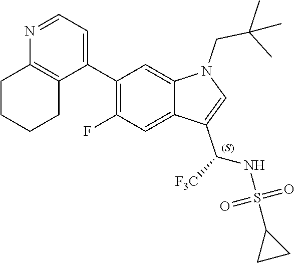

5,6,7,8-tetrahydroquinolin-4-yl trifluoromethanesulfonate (60 mg, 170.67 μmol), N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (151.44 mg, 170.67 μmol) Na₂CO₃ (36.18 mg, 341.33 μmol, 14.30 uL) and palladium;triphenylphosphane (39.44 mg, 34.13 μmol) were added to the sealed tube (20 ml). Then dioxane (4 mL) and H₂O (1 mL) were added to the mixture. The mixture was stirred at 100° C. for 2 h. Water (5 mL) was added to the mixture. The mixture was extracted with EA (50 ml). The organic layer was dried over anhydrous Na₂SO₄, filtered and concentrated. The residue obtained was purified by prep-HPLC to give N-[(1S)-1-[1-(2,2-dimethylpropyl)-5-fluoro-6-(5,6,7,8-tetrahydroquinolin-4-yl)indol-3-yl]-2,2,2-trifluoro-ethyl]cyclopropanesulfonamide (10 mg, 18.60 μmol, 10.90% yield) as a white solid. MS Found: 537.8 [M+H]⁺: 1H NMR (400 MHz, MeOD) δ 8.31 (d, J=5.0 Hz, 1H), 7.56 (d, J=12.3 Hz, 2H), 7.35 (d, J=5.9 Hz, 1H), 7.12 (d, J=5.0 Hz, 1H), 5.42 (q, J=7.9 Hz, 1H), 4.01 (s, 2H), 2.98 (t, J=6.5 Hz, 2H), 2.59 (s, 2H), 2.33 (ddd, J=9.5, 6.4, 4.0 Hz, 1H), 1.92 (dt, J=12.8, 6.5 Hz, 2H), 1.74 (s, 2H), 1.09-0.95 (m, 11H), 0.85-0.71 (m, 2H).

### Example 379: N-[(1S)-1-[1-(2,2-dimethylpropyl)-5-fluoro-6-pyrimidin-4-yl-indol-3-yl]-2,2,2-trifluoro-ethyl]cyclopropanesulfonamide (379)

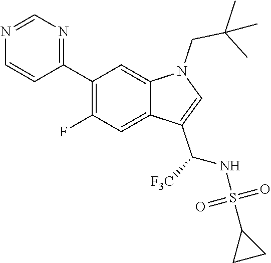 [SMILES: CC(C)(C)Cn1cc([C@H](NS(=O)(=O)C2CC2)C(F)(F)F)c2cc(F)c(-c3ccncn3)cc21]

### Step 1: 6-bromo-5-fluoro-1-neopentyl-1H-indole

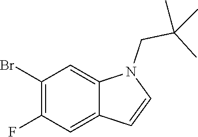 [SMILES: CC(C)(C)Cn1ccc2cc(F)c(Br)cc21]

6-Bromo-5-fluoro-1H-indole (10 g, 46.72 mmol) and potassium iodide (15.51 g, 93.44 mmol) in DMF (10 mL) was added to a suspension of NaH (7.47 g, 186.89 mmol, 60% purity) in anhydrous DMF (100 mL) in ice-water under nitrogen. The mixture was stirred for 1 h at room temperature then cooled with an ice batch, at which time, 1-bromo-2,2-dimethyl-propane (14.11 g, 93.44 mmol, 11.76 mL) was added and the resulting mixture was stirred at 40° C. overnight under nitrogen. After quenching with water the reaction mixture was partitioned between ethyl acetate (50 mL) and water (50 mL). The aqueous layer was extracted using ethyl acetate (2×50 mL), the combined organic layers were washed sequentially with sat. NH₄Cl and brine, then dried over anhydrous sodium sulfate. After the solvent was removed in vacuo, flash chromatography (silica gel, 100% Petroleum ether) yielded 6-bromo-1-(2,2-dimethylpropyl)-5-fluoro-indole (15.8 g, 55.60 mmol, 119.01% yield) as red brown oil.

### Step 2: (S,E)-2-methyl-N-(2,2,2-trifluoroethylidene)propane-2-sulfinamide

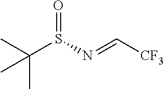

To a mixture of 2,2,2-trifluoroethane-1,1-diol (8 g, 51.71 mmol, 5.33 mL) and (S)-2-methylpropane-2-sulfinamide (8.15 g, 67.22 mmol) in anhydrous CH₂Cl₂ (120 mL) was added anhydrous MgSO₄ (11 g) and 4A Molecular sieves (30 g). The mixture was heated at 40° C. oil bath overnight. The mixture was cooled to RT and filtered. The filtrate solution of (S,E)-2-methyl-N-(2,2,2-trifluoroethylidene)propane-2-sulfinamide in DCM was used with no further purification.

### Step 3: (S)—N—((S)-1-(6-bromo-5-fluoro-1-neopentyl-1H-indol-3-yl)-2,2,2-trifluoroethyl)-2-methylpropane-2-sulfinamide

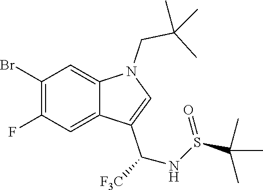 [SMILES: CC(C)(C)Cn1cc([C@H](N[S@@](=O)C(C)(C)C)C(F)(F)F)c2cc(F)c(Br)cc21]

The mixture of 6-bromo-1-(2,2-dimethylpropyl)-5-fluoro-indole (10.8 g, 38.01 mmol) in anhydrous CH₂Cl₂ (50 mL) was cooled to −10° C., at which time BF₃·OEt₂ (3.05 mL, 1.3 eq) was added slowly, followed by addition of a solution of (S,E)-2-methyl-N-(2,2,2-trifluoroethylidene)propane-2-sulfinamide in DCM. The mixture was then continued to stir for 2 h and then the mixture was diluted with DCM, followed by water. The aqueous layer was extracted with DCM twice. Organic layers were combined and washed with brine and concentrated in vacuo to obtain the crude product, which was purified by silica gel column (EA/PE=0-80%) to give (S)—N-[(1S)-1-[6-bromo-1-(2,2-dimethylpropyl)-5-fluoro-indol-3-yl]-2,2,2-trifluoro-ethyl]-2-methyl-propane-2-sulfinamide (12.47 g, 25.69 mmol) as yellow oil.

### Step 4: (S)-1-(6-bromo-5-fluoro-1-neopentyl-1H-indol-3-yl)-2,2,2-trifluoroethanamine

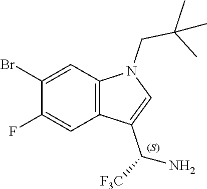

A mixture of (S)—N-[(1S)-1-[6-bromo-1-(2,2-dimethylpropyl)-5-fluoro-indol-3-yl]-2,2,2-trifluoro-ethyl]-2-methyl-propane-2-sulfinamide (11.97 g, 24.66 mmol) in 4M HCl(g)/MeOH (50 mL) was stirred at RT for 3 h. The mixture was concentrated, the residue was added sat. aqueous NaHCO₃ and EtOAc, stirred and separated, the aqueous was extracted with EtOAc×2. The combined organic phases were washed with brine, dried with anhydrous Na₂SO₄ and filtered. The filtrate was concentrated to give the crude product, which was purified by prep-HPLC to get (1S)-1-[6-bromo-1-(2,2-dimethylpropyl)-5-fluoro-indol-3-yl]-2,2,2-trifluoro-ethanamine (4.08 g, 10.70 mmol, 43.40% yield) as white solids. ¹H NMR (400 MHz, d₆-DMSO) δ 7.96 (d, J=5.9 Hz, 1H), 7.66 (d, J=9.9 Hz, 1H), 7.54 (s, 1H), 4.85 (d, J=7.6 Hz, 1H), 4.00 (s, 2H), 3.7-3.8 (br s, 2H), 0.91 (s, 9H). LCMS: 365.8 m/z [M+H]⁺.

### Step 5: N-[(1S)-1-[6-bromo-1-(2,2-dimethylpropyl)-5-fluoro-indol-3-yl]-2,2,2-trifluoro-ethyl]cyclopropanesulfonamide

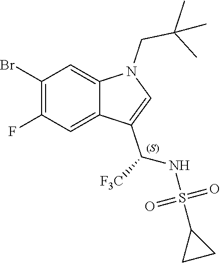

A mixture of (1S)-1-[6-bromo-1-(2,2-dimethylpropyl)-5-fluoro-indol-3-yl]-2,2,2-trifluoro-ethanamine (1.3 g, 3.41 mmol) and cyclopropanesulfonyl chloride (1.44 g, 10.23 mmol, 1.04 mL) in pyridine (9 mL) was stirred at RT overnight. The mixture was acidified with 2N HCl, then was extracted with EtOAc (3×40 ml). The combined organic phases were washed with Sat. NH₄Cl and brine, dried with Na₂SO₄, filtered and concentrated to get N-[(1S)-1-[6-bromo-1-(2,2-dimethylpropyl)-5-fluoro-indol-3-yl]-2,2,2-trifluoro-ethyl]cyclopropanesulfonamide (1.80 g, 3.71 mmol) as yellow solids.

### Step 6: N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide

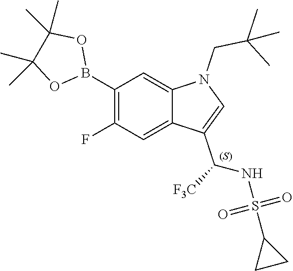

A mixture of N-[(1S)-1-[6-bromo-1-(2,2-dimethylpropyl)-5-fluoro-indol-3-yl]-2,2,2-trifluoro-ethyl]cyclopropanesulfonamide (611 mg, 1.26 mmol), 4,4,5,5-tetramethyl-2-(4,4,5,5-tetramethyl-1,3,2-dioxaborolan-2-yl)-1,3,2-dioxaborolane (479.53 mg, 1.89 mmol), Pd(dppf)Cl2 (368.78 mg, 0.504 mmol) and KOAc (370.65 mg, 3.78 mmol) in anhydrous dioxane (15 mL) was stirred at 80° C. overnight under Nitrogen. The completion of reaction was monitored by lcms. The mixture was concentrated, the residue was purified by silica gel column (EA/PE=0-25%) to get N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (790 mg, 1.48 mmol) as yellow gum, directly next step.

### Step 7: N-[(1S)-1-[1-(2,2-dimethylpropyl)-5-fluoro-6-pyrimidin-4-yl-indol-3-yl]-2,2,2-trifluoro-ethyl]cyclopropanesulfonamide

A mixture of 4-bromopyrimidine;hydrobromide (85 mg, 354.32 umol), N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (408.72 mg, 460.62 umol), Pd(PPh₃)₄ (163.78 mg, 141.73 umol) and K₂CO₃ (293.82 mg, 2.13 mmol) in dioxane (4 mL) and water (0.7 mL) was stirred at 80° C. overnight under Nitrogen. The completion of reaction was monitored by lcms. The mixture was added water and EtOAc, the mixture was stirred and separated, the aqueous phase was extracted with EtOAc twice, the combined organic phases were washed with brine and dried with Na₂SO₄, filtered and concentrated to get brown oil, which was purified by silica gel column (EA/PE=0-50%) to get yellow oil. which was purified by prep-HPLC to get N-[(1S)-1-[1-(2,2-dimethylpropyl)-5-fluoro-6-pyrimidin-4-yl-indol-3-yl]-2,2,2-trifluoro-ethyl]cyclopropanesulfonamide (15.4 mg, 31.78 umol) as light-yellow solid. ¹H NMR (400 MHz, CDCl₃) δ 9.29 (d, J=1.0 Hz, 1H), 8.75 (d, J=5.4 Hz, 1H), 8.25 (d, J=6.2 Hz, 1H), 7.92 (dd, J=3.8, 1.6 Hz, 1H), 7.52 (d, J=12.4 Hz, 1H), 7.30 (s, 1H), 5.38 (dd, J=15.1, 7.6 Hz, 1H), 5.16 (s, 1H), 3.97 (d, J=2.2 Hz, 2H), 2.47 (tt, J=8.0, 4.9 Hz, 1H), 1.29-1.10 (m, 2H), 1.05-0.83 (m, 11H). LCMS: 484.8 m/z [M+H]⁺.

### Example 380: N-[(1S)-1-[1-(2,2-dimethylpropyl)-5-fluoro-6-(1H-pyrazol-5-yl)indol-3-yl]-2,2,2-trifluoro-ethyl]cyclopropanesulfonamide (380)

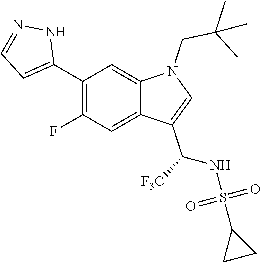 [SMILES: CC(C)(C)Cn1cc([C@H](NS(=O)(=O)C2CC2)C(F)(F)F)c2cc(F)c(-c3ccn[nH]3)cc21]

A mixture of N-[(1S)-1-[6-bromo-1-(2,2-dimethylpropyl)-5-fluoro-indol-3-yl]-2,2,2-trifluoro-ethyl]cyclopropanesulfonamide (73 mg, 150.41 umol), 1H-pyrazol-5-ylboronic acid (25.25 mg, 225.62 umol), Pd(dppf)Cl2 (368.78 mg, 0.504 mmol) and disodium carbonate (47.83 mg, 451.24 umol) in 1,4-dioxane (3 mL) and water (0.7 mL) was stirred at 80° C. overnight under Nitrogen. the mixture was concentrated, the residue was added water, the aqueous phase was extracted with EtOAc twice. the combined organic phases were washed with brine, dried with anhydrous Na₂SO₄ and filtered. The filtrate was concentrated to get oil, which was purified by prep-HPLC to get N-[(1S)-1-[1-(2,2-dimethylpropyl)-5-fluoro-6-(1H-pyrazol-5-yl)indol-3-yl]-2,2,2-trifluoro-ethyl]cyclopropanesulfonamide (17.5 mg, 37.04 umol, 24.62% yield) as a white solid. ¹H NMR (400 MHz, MeOD) δ 7.90 (d, J=51.1 Hz, 1H), 7.62 (d, J=80.6 Hz, 3H), 6.73 (s, 1H), 5.39 (d, J=7.8 Hz, 1H), 4.04 (s, 2H), 2.29 (s, 1H), 1.30 (s, 1H), 0.99 (d, J=23.3 Hz, 10H), 0.83-0.63 (m, 2H). LCMS: 472.8 m/z [M+H]⁺.

### Example 381: N-[(1S)-1-[1-(2,2-dimethylpropyl)-5-fluoro-6-(4-methyl-1H-pyrazol-5-yl)indol-3-yl]-2,2,2-trifluoro-ethyl]cyclopropanesulfonamide (381)

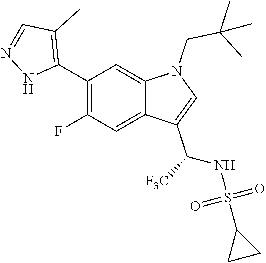 [SMILES: Cc1cn[nH]c1-c1cc2c(cc1F)c([C@H](NS(=O)(=O)C1CC1)C(F)(F)F)cn2CC(C)(C)C]

A mixture of 3-bromo-4-methyl-1H-pyrazole (120 mg, 745.34 umol), N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (436.50 mg, 819.87 umol), Pd(PPh₃)₄ (344.52 mg, 298.14 umol) and K₂CO₃ (309.04 mg, 2.24 mmol) in dioxane (5 mL) and water (0.8 mL) was stirred at 80° C. overnight under Nitrogen. The completion of reaction was monitored by lcms. The mixture was added water and EtOAc, the mixture was stirred and separated, the aqueous phase was extracted with EtOAc twice, the combined organic phases were washed with brine and dried with Na₂SO₄, filtered and concentrated to get brown oil, which was purified by silica gel column (EA/PE=0-50%) to get yellow oil. which was purified by prep-HPLC twice to get N-[(1S)-1-[1-(2,2-dimethylpropyl)-5-fluoro-6-(4-methyl-1H-pyrazol-5-yl)indol-3-yl]-2,2,2-trifluoro-ethyl]cyclopropanesulfonamide (9.03 mg, 18.56 umol) as a white solid. ¹H NMR (400 MHz, CDCl₃) δ 7.53-7.45 (m, 2H), 7.38 (d, J=5.8 Hz, 1H), 7.24 (s, 1H), 5.36 (d, J=7.6 Hz, 2H), 3.85-3.65 (m, 2H), 2.48 (ddd, J=9.7, 6.4, 4.0 Hz, 1H), 2.18 (s, 3H), 1.29-1.21 (m, 1H), 1.19-1.11 (m, 1H), 1.05-0.88 (m, 10H). LCMS: 486.8 m/z [M+H]⁺.

### Example 382: (S)—N-(2,2,2-trifluoro-1-(1-neopentyl-6-(pyrimidin-2-yl)-1H-indol-3-yl)ethyl)cyclopropanesulfonamide (382)

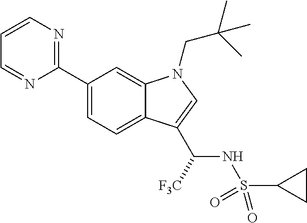 [SMILES: CC(C)(C)Cn1cc([C@H](NS(=O)(=O)C2CC2)C(F)(F)F)c2ccc(-c3ncccn3)cc21]

A mixture of (S)—N-(1-(6-bromo-1-neopentyl-1H-indol-3-yl)-2,2,2-trifluoroethyl)cyclopropanesulfonamide (0.0737 g, 0.158 mmol), 2-(tributylstannyl)pyrimidine (0.088 g, 0.238 mmol), CsF (0.048 g, 1.16 mmol), CuI (0.003 g, 0.016 mmol) and Pd(PPh₃)₄ (0.018 g, 0.016 mmol) in DMF was degassed and heated overnight in a 140° C. oil bath. The mixture was cooled to rt and filtered. The crude was purified by prep-HPLC to obtain the title compound as a TFA salt. ESI-MS (m/z): 467.4 [M+1]⁺.

### Example 383: (S)—N-(2,2,2-trifluoro-1-(1-neopentyl-6-(pyridin-3-yl)-1H-indol-3-yl)ethyl)cyclopropanesulfonamide (383)

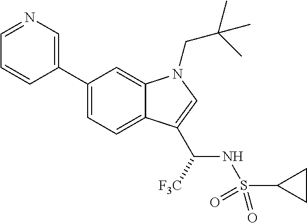 [SMILES: CC(C)(C)Cn1cc([C@H](NS(=O)(=O)C2CC2)C(F)(F)F)c2ccc(-c3cccnc3)cc21]

The title compound was prepared following the same general protocol as described for Step 5, Example 1, using (S)—N-(1-(6-bromo-1-neopentyl-1H-indol-3-yl)-2,2,2-trifluoroethyl)cyclopropanesulfonamide and pyridin-3-ylboronic acid. ESI-MS (m/z): 466.30 [M+1]⁺.

### Example 384: (S)—N-(1-(6-(3-cyanopyridin-4-yl)-1-neopentyl-1H-indol-3-yl)-2,2,2-trifluoroethyl)cyclopropanesulfonamide (384)

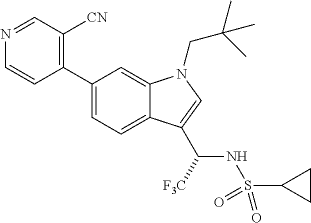 [SMILES: CC(C)(C)Cn1cc([C@H](NS(=O)(=O)C2CC2)C(F)(F)F)c2ccc(-c3ccncc3C#N)cc21]

The title compound was prepared following the same general protocol as described for Step 5, Example 1, using (S)—N-(1-(6-bromo-1-neopentyl-1H-indol-3-yl)-2,2,2-trifluoroethyl)cyclopropanesulfonamide and 4-(4,4,5,5-tetramethyl-1,3-dioxolan-2-yl)nicotinonitrile. ESI-MS (m/z): 491.42 [M+1]⁺.

### Example 385: (S)—N-(2,2,2-trifluoro-1-(6-(2-methylimidazo[1,2-a]pyridin-3-yl)-1-neopentyl-1H-indol-3-yl)ethyl)cyclopropanesulfonamide (385)

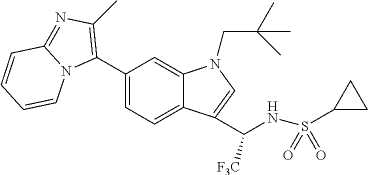 [SMILES: Cc1nc2ccccn2c1-c1ccc2c([C@H](NS(=O)(=O)C3CC3)C(F)(F)F)cn(CC(C)(C)C)c2c1]

### Step 1: (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide

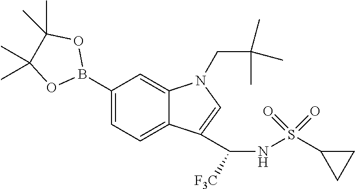 [SMILES: CC(C)(C)Cn1cc([C@H](NS(=O)(=O)C2CC2)C(F)(F)F)c2ccc(B3OC(C)(C)C(C)(C)O3)cc21]

The title compound was prepared following the same general protocol as described for Step 1, Example B-18, using (S)—N-(1-(6-bromo-1-neopentyl-1H-indol-3-yl)-2,2,2-trifluoroethyl)cyclopropanesulfonamide. ¹HNMR (CDCl₃, 400 MHz) δ 7.84 (s, 1H), 7.70 (d, J=8.0 Hz, 1H), 7.60 (d, J=8.0 Hz, 1H), 7.25 (s, 1H), 5.43-5.35 (q, J=8.0 Hz, 1H), 5.13 (d, J=8.0 Hz, 1H), 3.95 (s, 2H), 2.48-2.42 (m, 1H), 1.86-1.53 (m, 2H), 1.48 (s, 12H), 0.99 (s, 9H), 0.98-0.96 (m, 2H)

### Step 2: (S)—N-(2,2,2-trifluoro-1-(6-(2-methylimidazo[1,2-a]pyridin-3-yl)-1-neopentyl-1H-indol-3-yl)ethyl)cyclopropanesulfonamide

The title compound was prepared following the same general protocol as described for Step 5, Example 1, using 3-bromo-2-methylimidazo[1,2-a]pyridine and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 8.08 (d, J=8.0 Hz, 1H), 7.88 (d, J=8.0 Hz, 1H), 7.55 (d, J=8.0 Hz, 1H), 7.32 (s, 1H), 7.28-7.23 (m, 2H), 7.14 (t, J=8.0 Hz, 1H), 7.69 (t, J=8.0 Hz, 1H), 5.68 (broad s, 1H), 5.49 (broad s, 1H), 3.89 (s, 2H), 2.48-2.42 (m, 1H), 2.45 (s, 3H), 1.09-0.93 (m, 2H), 0.92-0.71 (m, 11H)

### Example 386: (S)—N-(1-(6-(2-cyano-3-fluorophenyl)-1-neopentyl-1H-indol-3-yl)-2,2,2-trifluoroethyl)cyclopropanesulfonamide (386)

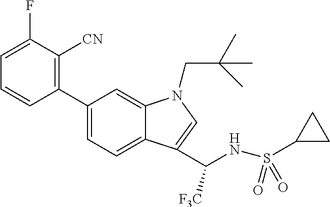 [SMILES: CC(C)(C)Cn1cc([C@H](NS(=O)(=O)C2CC2)C(F)(F)F)c2ccc(-c3cccc(F)c3C#N)cc21]

The title compound was prepared following the same general protocol as described for Step 5, Example 1, using 2-bromo-6-fluorobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. ¹HNMR (CDCl₃, 400 MHz) δ 7.82 (d, J=8.0 Hz, 1H), 7.65-7.59 (m, 1H), 7.53 (s, 1H), 7.40-7.28 (m, 3H), 7.17 (t, J=8.0 Hz, 1H), 7.69 (t, J=8.0 Hz, 1H), 5.44 (q, J=8.0 Hz, 1H), 5.24 (d, J=8.0 Hz, 1H), 3.88 (s, 2H), 2.47-2.41 (m, 1H), 1.27-1.02 (m, 2H), 0.96-0.88 (m, 11H)

### Example 387: (S)—N-(1-(6-(3-cyano-4-fluorophenyl)-1-neopentyl-1H-indol-3-yl)-2,2,2-trifluoroethyl)cyclopropanesulfonamide (387)

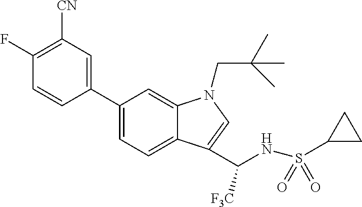 [SMILES: CC(C)(C)Cn1cc([C@H](NS(=O)(=O)C2CC2)C(F)(F)F)c2ccc(-c3ccc(F)c(C#N)c3)cc21]

The title compound was prepared following the same general protocol as described for Step 5, Example 1, using 5-bromo-2-fluorobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.82-7.79 (m, 3H), 7.43-7.26 (m, 4H), 5.44 (q, J=8.0 Hz, 1H), 5.17 (d, J=8.0 Hz, 1H), 3.95 (s, 2H), 2.47-2.41 (m, 1H), 1.27-1.10 (m, 2H), 0.95 (s, 9H), 0.94-0.89 (m, 2H).

### Example 388: (S)—N-(1-(6-(2-chloro-6-methylphenyl)-1-neopentyl-1H-indol-3-yl)-2,2,2-trifluoroethyl)cyclopropanesulfonamide (388)

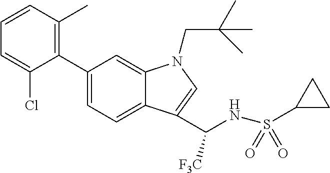 [SMILES: Cc1cccc(Cl)c1-c1ccc2c([C@H](NS(=O)(=O)C3CC3)C(F)(F)F)cn(CC(C)(C)C)c2c1]

The title compound was prepared following the same general protocol as described for Step 5, Example 1, using 2-bromo-1-chloro-3-methylbenzene and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.78 (t, J=8.0 Hz, 1H), 7.34-7.32 (m, 1H), 7.21-7.18 (m, 4H), 7.99 (d, J=8.0 Hz, 1H), 5.47-5.41 (m, 1H), 5.17 (dd, J=24.0 Hz, 8.0 Hz, 1H), 3.90 (s, 2H), 2.49-2.42 (m, 1H), 2.08 (d, J=4.0 Hz, 3H), 1.27-1.02 (m, 2H), 0.99 (s, 9H), 0.94-0.88 (m, 2H).

### Example 389: (S)—N-(1-(6-(2-cyano-6-(trifluoromethyl)phenyl)-1-neopentyl-1H-indol-3-yl)-2,2,2-trifluoroethyl)cyclopropanesulfonamide (389)

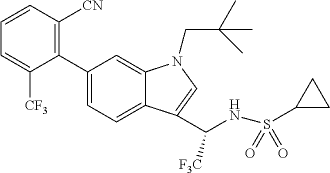 [SMILES: CC(C)(C)Cn1cc([C@H](NS(=O)(=O)C2CC2)C(F)(F)F)c2ccc(-c3c(C#N)cccc3C(F)(F)F)cc21]

The title compound was prepared following the same general protocol as described for Step 5, Example 1, using 2-bromo-3-(trifluoromethyl)benzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.98 (d, J=8.0 Hz, 1H), 7.91 (d, J=8.0 Hz, 1H), 7.80 (t, J=8.0 Hz, 1H), 7.61 (t, J=8.0 Hz, 1H), 7.29 (d, J=8.0 Hz, 2H), 7.08 (d, J=8.0 Hz, 1H), 5.46-5.40 (m, 1H), 5.19 (t, J=8.0 Hz, 1H), 3.90 (m, 2H), 2.49-2.42 (m, 1H), 1.18-1.04 (m, 2H), 0.97 (s, 9H), 0.90-0.78 (m, 2H).

### Example 390: (S)—N-(1-(6-(2-cyano-6-methylphenyl)-1-neopentyl-1H-indol-3-yl)-2,2,2-trifluoroethyl)cyclopropanesulfonamide (390)

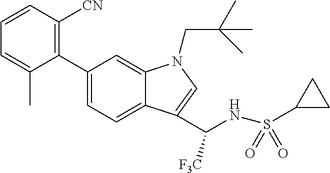 [SMILES: Cc1cccc(C#N)c1-c1ccc2c([C@H](NS(=O)(=O)C3CC3)C(F)(F)F)cn(CC(C)(C)C)c2c1]

The title compound was prepared following the same general protocol as described for Step 5, Example 1, using 2-bromo-3-methylbenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.80 (broad s, 1H), 7.58 (d, J=8.0 Hz, 1H), 7.50 (d, J=4.0 Hz, 1H), 7.34 (t, J=8.0 Hz, 1H), 7.27 (d, J=4.0 Hz, 2H), 7.05 (d, J=8.0 Hz, 1H), 5.48-5.40 (m, 1H), 5.29-5.14 (m, 1H), 3.89 (broad s, 2H), 2.49-2.42 (m, 1H), 2.17 (s, 3H), 1.19-1.10 (m, 2H), 0.99 (s, 9H), 0.89-0.83 (m, 2H).

### Example 391: N-[(1S)-2,2,2-trifluoro-1-[5-fluoro-1-(2-methoxy-2-methyl-propyl)-6-[2-(trifluoromethyl)phenyl]indol-3-yl]ethyl]cyclobutanesulfonamide (391)

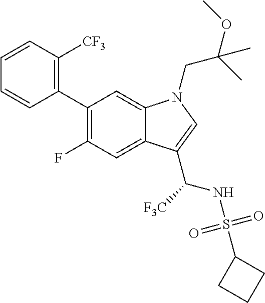 [SMILES: COC(C)(C)Cn1cc([C@H](NS(=O)(=O)C2CCC2)C(F)(F)F)c2cc(F)c(-c3ccccc3C(F)(F)F)cc21]

### Step 1: cyclobutanesulfonyl chloride

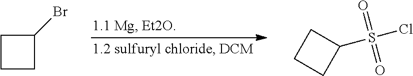 [SMILES: BrC1CCC1 ; O=S(=O)(Cl)C1CCC1]

To a suspension of Magnesium (306 mg, 12.59 mmol, 175.86 uL) in Diethyl ether (8 mL) was added a solution of bromocyclobutane (1 g, 7.41 mmol) in Diethyl ether (8 mL) in several small portions at 25° C. The mixture was stirred at 35° C. for 15 min under N2. Then the mixture was refluxed for 1 h. The suspension was added in small portions to an ice-cold solution of sulfuryl chloride (3 g, 22.23 mmol) in DCM (12 mL). The suspension was warmed to RT, and the volatiles were removed under reduced pressure at RT for 15 min and at 42° C. for 20 min. The residue obtained was extracted with hexane (50 ml) and filtered. The filtrates were dried over anhydrous Na₂SO₄, filtered and concentrated to give cyclobutanesulfonyl chloride (0.3 g, 1.94 mmol, 26.19% yield) as a light-yellow oil.

### Step 2: N-[(1S)-2,2,2-trifluoro-1-[5-fluoro-1-(2-methoxy-2-methyl-propyl)-6-[2-(trifluoromethyl)phenyl]indol-3-yl]ethyl]cyclobutanesulfonamide

To a mixture of (1S)-2,2,2-trifluoro-1-[5-fluoro-1-(2-methoxy-2-methyl-propyl)-6-[2-(trifluoromethyl)phenyl]indol-3-yl]ethanamine (0.1 g, 216.26 μmol) in Pyridine (5 mL) at RT was added cyclobutanesulfonyl chloride (300.94 mg, 1.95 mmol). The mixture was stirred at RT overnight. Then the reaction was stirred at 90° C. in microwave for 1 h. The mixture was diluted with EtOAc, washed with 2N HCl, water, sat. NaHCO₃ and brine and dried over Na₂SO₄. Solvent was removed in vacuo to obtain the crude which was purified by prep-HPLC to afford N-[(1S)-2,2,2-trifluoro-1-[5-fluoro-1-(2-methoxy-2-methyl-propyl)-6-[2-(trifluoromethyl)phenyl]indol-3-yl]ethyl]cyclobutanesulfonamide (4.17 mg, 7.18 μmol) as an orange solid. MS Found: 580.8 [M+H]⁺. 1H NMR (400 MHz, MeOD) δ 7.80 (d, J=7.6 Hz, 1H), 7.66 (t, J=7.2 Hz, 1H), 7.58 (t, J=7.6 Hz, 1H), 7.53 (s, 1H), 7.51-7.37 (m, 3H), 5.33 (d, J=4.7 Hz, 1H), 4.15 (q, J=19.2 Hz, 2H), 3.76-3.55 (m, 1H), 3.21 (s, 3H), 2.41-2.25 (m, 2H), 2.08-1.98 (m, 2H), 1.81 (d, J=5.8 Hz, 2H), 1.14 (t, J=14.2 Hz, 6H).

### Example 392: [(1S)-1-[1-[(1-cyanocyclopropyl)methyl]-5-fluoro-6-[2-(trifluoromethyl)phenyl]indol-3-yl]-2,2,2-trifluoro-ethyl]cyclobutanesulfonamide (392)

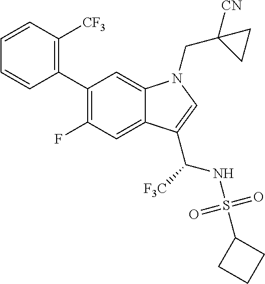 [SMILES: N#CC1(Cn2cc([C@H](NS(=O)(=O)C3CCC3)C(F)(F)F)c3cc(F)c(-c4ccccc4C(F)(F)F)cc32)CC1]

The mixture of 1-[[3-[(1S)-1-amino-2,2,2-trifluoro-ethyl]-5-fluoro-6-[2-(trifluoromethyl)phenyl]indol-1-yl]methyl]cyclopropanecarbonitrile (100 mg, 219.60 umol) and cyclobutanesulfonyl chloride (67.91 mg, 439.20 umol) in pyridine (1 mL) were stirred at 90° C. for 2 h under microwave. LCMS showed 20% desired product was observed. The reaction was washed by 1 N HCl and extracted with EA (50 mL×2). The combined organic layers were dried over anhydrous Na₂SO₄, filtered and concentrated. The residue obtained was purified by prep-HPLC (base) to give N-[(1S)-1-[1-[(1-cyanocyclopropyl)methyl]-5-fluoro-6-[2-(trifluoromethyl)phenyl]indol-3-yl]-2,2,2-trifluoro-ethyl]cyclobutanesulfonamide (3.5 mg, 6.10 umol) as a yellow solid. ¹H NMR (400 MHz, MeOD) δ 7.83 (d, J=7.7 Hz, 1H), 7.75-7.66 (m, 2H), 7.65-7.53 (m, 2H), 7.49 (d, J=5.9 Hz, 2H), 5.47-5.33 (m, 1H), 4.52 (s, 2H), 4.35-4.20 (m, 1H), 3.82-3.62 (m, 1H), 2.42-2.27 (m, 2H), 2.04 (s, 2H), 1.85 (d, J=6.3 Hz, 2H), 1.30-1.10 (m, 3H). LCMS: 590.8 m/z [M+H₂O]⁺.

### Example 393: N-(2-(6-(3-fluoro-2-(trifluoromethyl)phenyl)-1-neopentyl-1H-indol-3-yl)ethyl)cyclopropanesulfonamide. (393)

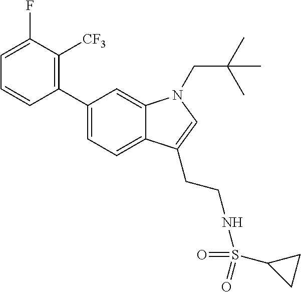 [SMILES: CC(C)(C)Cn1cc(CCNS(=O)(=O)C2CC2)c2ccc(-c3cccc(F)c3C(F)(F)F)cc21]

### Step 1: 2-(6-bromo-1H-indol-3-yl)-2-oxoacetamide

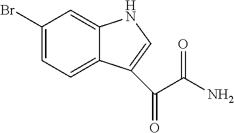 [SMILES: NC(=O)C(=O)c1c[nH]c2cc(Br)ccc12]

The title compound was prepared following the same general protocol as described for Step 2, Example 1, using 6-bromo-1H-indole. ESI-MS (m/z): 266.45, 248.57 [M+1]⁺.

### Step 2: 2-(6-bromo-1-pivaloyl-1H-indol-3-yl)-2-oxoacetamide

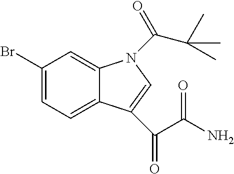 [SMILES: CC(C)(C)C(=O)n1cc(C(=O)C(N)=O)c2ccc(Br)cc21]

To a mixture of 2-(6-bromo-1H-indol-3-yl)-2-oxoacetamide (4.588 g, 17.178 mmol), TEA (7.2 mL, 51.534 mmol) and DMAP (0.42 g, 3.44 mmol) in DCM (180 mL) at 0° C. was added pivaloyl chloride (4.2 mL, 34.335 mmol). The reaction was monitored by anal. HPLC for completion. The mixture was concentrated and redissolved in EtOAc, washed with 1N HCl, water and saturated NaHCO₃ and brine, and dried over Na₂SO₄. The solvent was removed and purified with silica gel to obtain the title compound. ESI-MS (m/z): 350.81, 352.23 [M+1]⁺.

### Step 3: 2-(6-bromo-1-neopentyl-1H-indol-3-yl)ethan-1-amine

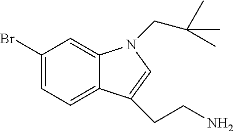 [SMILES: CC(C)(C)Cn1cc(CCN)c2ccc(Br)cc21]

The title compound was prepared following the same general protocol as described for Step 3, Example 1, 2-(6-bromo-1-pivaloyl-1H-indol-3-yl)-2-oxoacetamide. ESI-MS (m/z): 308.79, 310.45 [M+1]⁺.

### Step 4: N-(2-(6-bromo-1-neopentyl-1H-indol-3-yl)ethyl)cyclopropanesulfonamide

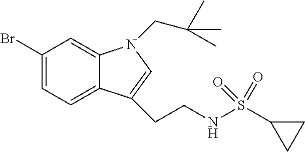 [SMILES: CC(C)(C)Cn1cc(CCNS(=O)(=O)C2CC2)c2ccc(Br)cc21]

The title compound was prepared following the same general protocol as described for Step 4, Example 1, 2-(6-bromo-1-neopentyl-1H-indol-3-yl)ethan-1-amine and cyclopropanesulfonyl chloride. HNMR (CDCl₃, 400 MHz) δ 7.55 (broad s, 1H), 7.45 (d, J=8.0 Hz, 1H), 7.22 (dd, J=8.0 4.0 Hz, 1H), 7.09 (broad s, 1H), 4.28 (t, J=8.0 Hz, 1H), 3.44 (q, J=8.0 Hz, 2H), 3.15 (s, 2H), 3.03 (t, J=8.0 Hz, 2H), 2.49-2.42 (m, 1H), 1.19-1.10 (m, 2H), 0.99-0.83 (m, 11H)

### Step 5: N-(2-(6-(3-fluoro-2-(trifluoromethyl)phenyl)-1-neopentyl-1H-indol-3-yl)ethyl)cyclopropanesulfonamide

The title compound was prepared following the same general protocol as described for Step 5, Example 1, using N-(2-(6-bromo-1-neopentyl-1H-indol-3-yl)ethyl)cyclopropanesulfonamide and 2-(3-fluoro-2-(trifluoromethyl)phenyl)-4,4,5,5-tetramethyl-1,3,2-dioxaborolane. HNMR (CDCl₃, 400 MHz) δ 7.59 (d, J=8.0 Hz, 1H), 7.56-7.48 (m, 1H), 7.32 (broad s, 1H), 7.24-7.12 (m, 3H), 7.04 (d, J=8.0 Hz, 1H), 4.30 (t, J=8.0 Hz, 1H), 3.52 (q, J=8.0 Hz, 2H), 3.14 (s, 2H), 3.08 (t, J=8.0 Hz, 2H), 2.39-2.32 (m, 1H), 1.18-1.10 (m, 2H), 0.99 (s, 9H), 0.98-0.83 (m, 2H)

### Example 394: N-(2-(6-(4-chloro-2-(trifluoromethyl)phenyl)-1-neopentyl-1H-indol-3-yl)ethyl)cyclopropanesulfonamide (394)

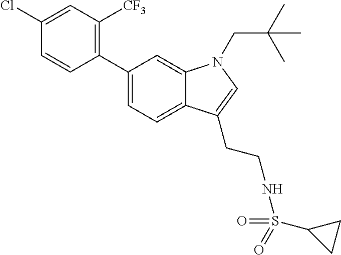 [SMILES: CC(C)(C)Cn1cc(CCNS(=O)(=O)C2CC2)c2ccc(-c3ccc(Cl)cc3C(F)(F)F)cc21]

The title compound was prepared following the same general protocol as described for Step 5, Example 1, using N-(2-(6-bromo-1-neopentyl-1H-indol-3-yl)ethyl)cyclopropanesulfonamide and (4-chloro-2-(trifluoromethyl)phenyl)boronic acid. ¹HNMR (CDCl₃, 400 MHz) δ 7.74 (d, J=4.0 Hz, 1H), 7.76-7.44 (m, 3H), 7.35 (d, J=8.0 Hz, 1H), 7.25-7.18 (m, 1H), 7.09 (s, 1H), 4.36 (t, J=8.0 Hz, 1H), 3.54-3.43 (m, 2H), 3.12 (s, 2H), 3.00 (t, J=8.0 Hz, 2H), 2.38-2.31 (m, 1H), 1.13-1.10 (m, 2H), 0.99 (s, 9H), 0.95-0.90 (m, 2H)

### Example 395: N-(2-(6-(4-fluoro-2-(trifluoromethyl)phenyl)-1-neopentyl-1H-indol-3-yl)ethyl)cyclopropanesulfonamide (395)

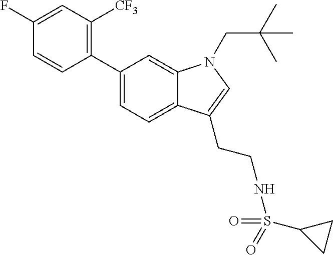 [SMILES: CC(C)(C)Cn1cc(CCNS(=O)(=O)C2CC2)c2ccc(-c3ccc(F)cc3C(F)(F)F)cc21]

The title compound was prepared following the same general protocol as described for Step 5, Example 1, using N-(2-(6-bromo-1-neopentyl-1H-indol-3-yl)ethyl)cyclopropanesulfonamide and (4-fluoro-2-(trifluoromethyl)phenyl)boronic acid. ¹HNMR (CDCl₃, 400 MHz) δ 7.76 (s, 1H), 7.59-7.51 (m, 3H), 7.19-7.17 (m, 2H), 7.05 (s, 1H), 4.21 (t, J=8.0 Hz, 1H), 3.53 (q, J=8.0 Hz, 2H), 3.16 (s, 2H), 3.19 (t, J=8.0 Hz, 2H), 2.38-2.30 (m, 1H), 1.14-1.10 (m, 2H), 0.99 (s, 9H), 0.95-0.90 (m, 2H)

### Example 396: N-(2-(6-(2-chloro-4-(trifluoromethyl)phenyl)-1-neopentyl-1H-indol-3-yl)ethyl)cyclopropanesulfonamide (396)

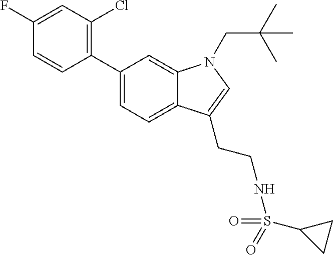 [SMILES: CC(C)(C)Cn1cc(CCNS(=O)(=O)C2CC2)c2ccc(-c3ccc(F)cc3Cl)cc21]

The title compound was prepared following the same general protocol as described for Step 5, Example 1, using N-(2-(6-bromo-1-neopentyl-1H-indol-3-yl)ethyl)cyclopropanesulfonamide and (2-chloro-4-(trifluoromethyl)phenyl)boronic acid. ¹HNMR (CDCl₃, 400 MHz) δ 7.76 (s, 1H), 7.65 (d, J=8.0 Hz, 1H), 7.59-7.51 (m, 3H), 7.19-7.17 (m, 2H), 4.21 (t, J=8.0 Hz, 1H), 3.53 (q, J=8.0 Hz, 2H), 3.16 (s, 2H), 3.19 (t, J=8.0 Hz, 2H), 2.38-2.30 (m, 1H), 1.14-1.10 (m, 2H), 0.99 (s, 9H), 0.95-0.90 (m, 2H)

### Example 397: N-(2-(6-(2,4-dichlorophenyl)-1-neopentyl-1H-indol-3-yl)ethyl)cyclopropanesulfonamide (397)

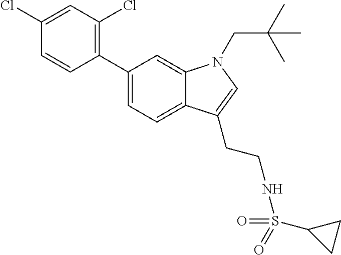 [SMILES: CC(C)(C)Cn1cc(CCNS(=O)(=O)C2CC2)c2ccc(-c3ccc(Cl)cc3Cl)cc21]

The title compound was prepared following the same general protocol as described for Step 5, Example 1, using N-(2-(6-bromo-1-neopentyl-1H-indol-3-yl)ethyl)cyclopropanesulfonamide and (2,4-dichlorophenyl)boronic acid. ¹HNMR (CDCl₃, 400 MHz) δ 7.73 (d, J=8.0 Hz, 1H), 7.52 (d, J=4.0 Hz, 1H), 7.46 (s, 1H), 7.34-7.32 (m, 2H), 7.18-7.14 (m, 2H), 4.21 (t, J=8.0 Hz, 1H), 3.53 (q, J=8.0 Hz, 2H), 3.15 (s, 2H), 3.09 (t, J=8.0 Hz, 2H), 2.40-2.32 (m, 1H), 1.16-1.12 (m, 2H), 0.96 (s, 9H), 0.95-0.86 (m, 2H)

### Example 398: N-(2-(6-(4-methyl-2-(trifluoromethyl)phenyl)-1-neopentyl-1H-indol-3-yl)ethyl)cyclopropanesulfonamide (398)

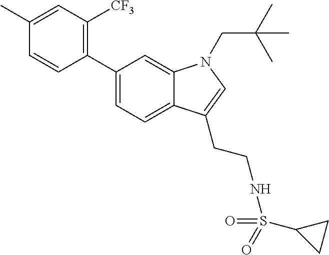 [SMILES: Cc1ccc(-c2ccc3c(CCNS(=O)(=O)C4CC4)cn(CC(C)(C)C)c3c2)c(C(F)(F)F)c1]

### Step 1: N-(2-(1-neopentyl-6-(4,4,5,5-tetramethyl-1,3,2-dioxaborolan-2-yl)-1H-indol-3-yl)ethyl)cyclopropanesulfonamide

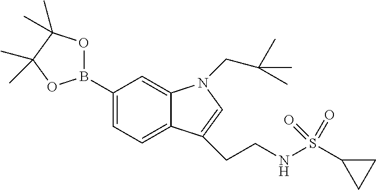 [SMILES: CC(C)(C)Cn1cc(CCNS(=O)(=O)C2CC2)c2ccc(B3OC(C)(C)C(C)(C)O3)cc21]

The title compound was prepared following the same general protocol as described for Step 1, Example B-18, using N-(2-(6-bromo-1-neopentyl-1H-indol-3-yl)ethyl)cyclopropanesulfonamide. ¹HNMR (CDCl₃, 400 MHz) δ 7.81 (s, 1H), 7.57-7.52 (m, 2H), 7.02 (s, 1H), 4.19 (t, J=8.0 Hz, 1H), 3.92 (s, 2H), 3.48 (q, J=8.0 Hz, 2H), 3.05 (t, J=8.0 Hz, 2H), 2.31-2.25 (m, 1H), 1.17 (s, 12H), 1.16-1.12 (m, 2H), 0.96 (s, 9H), 0.95-0.86 (m, 2H)

### Step 2: N-(2-(6-(4-methyl-2-(trifluoromethyl)phenyl)-1-neopentyl-1H-indol-3-yl)ethyl)cyclopropanesulfonamide

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 1-bromo-4-methyl-2-(trifluoromethyl)benzene. ¹HNMR (CDCl₃, 400 MHz) δ 7.57-7.55 (m, 2H), 7.35 (d, J=8.0 Hz, 1H), 7.27 (s, 2H), 7.04 (d, J=8.0 Hz, 1H), 7.00 (s, 1H), 4.25 (t, J=8.0 Hz, 1H), 3.85 (s, 2H), 3.52 (q, J=8.0 Hz, 2H), 3.07 (t, J=8.0 Hz, 2H), 2.45 (s, 3H), 2.3-2.25 (m, 1H), 1.16-1.12 (m, 2H), 0.96 (s, 9H), 0.95-0.86 (m, 2H)

### Example 399: N-(2-(6-(2-cyano-3-(trifluoromethyl)phenyl)-1-neopentyl-1H-indol-3-yl)ethyl)cyclopropanesulfonamide (399)

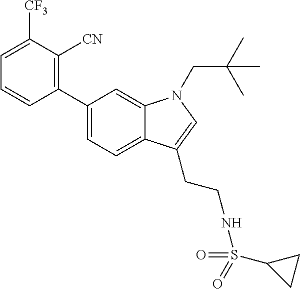 [SMILES: CC(C)(C)Cn1cc(CCNS(=O)(=O)C2CC2)c2ccc(-c3cccc(C(F)(F)F)c3C#N)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-6-(trifluoromethyl)benzonitrile. HNMR (CDCl₃, 400 MHz) δ 7.79-7.73 (m, 2H), 7.69 (d, J=8.0 Hz, 1H), 7.21 (d, J=8.0 Hz, 1H), 7.09 (s, 1H), 4.24 (t, J=8.0 Hz, 1H), 3.92 (s, 2H), 3.50 (q, J=8.0 Hz, 2H), 3.09 (t, J=8.0 Hz, 2H), 2.37-2.31 (m, 1H), 1.15-1.11 (m, 2H), 0.99 (s, 9H), 0.96-0.89 (m, 2H)

### Example 400: N-(2-(6-(2-cyano-4-(trifluoromethyl)phenyl)-1-neopentyl-1H-indol-3-yl)ethyl)cyclopropanesulfonamide (400)

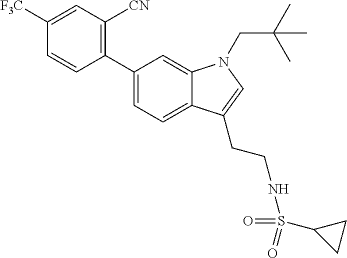 [SMILES: CC(C)(C)Cn1cc(CCNS(=O)(=O)C2CC2)c2ccc(-c3ccc(C(F)(F)F)cc3C#N)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-5-(trifluoromethyl)benzonitrile. ¹HNMR (CDCl₃, 400 MHz) δ 8.03 (s, 1H), 7.87 (d, J=8.0 Hz, 1H), 7.21-7.69 (m, 2H), 7.55 (s, 1H), 7.37 (d, J=8.0 Hz, 1H), 7.09 (s, 1H), 4.28 (t, J=8.0 Hz, 1H), 3.92 (s, 2H), 3.50 (q, J=8.0 Hz, 2H), 3.08 (t, J=8.0 Hz, 2H), 2.38-2.32 (m, 1H), 1.14-1.11 (m, 2H), 0.99 (s, 9H), 0.96-092 (m, 2H)

### Example 401: N-(2-(5-(4-chloro-2-cyanophenyl)-3-neopentyl-1H-indol-1-yl)ethyl)cyclopropanesulfonamide (401)

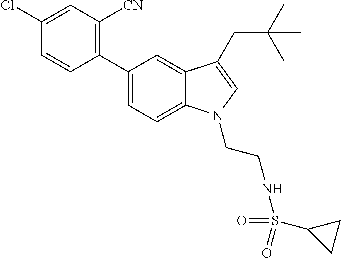 [SMILES: CC(C)(C)Cc1cn(CCNS(=O)(=O)C2CC2)c2ccc(-c3ccc(Cl)cc3C#N)cc12]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-5-chlorobenzonitrile. HNMR (CDCl₃, 400 MHz) δ 7.73-7.71 (m, 2H), 7.61 (dd, J=8.0, 4.0 Hz, 1H), 7.49 (d, J=8.0 Hz, 1H), 7.42 (d, J=8.0 Hz, 1H), 7.35 (dd, J=8.0, 4.0 Hz, 1H), 6.99 (s, 1H), 4.38 (t, J=8.0 Hz, 1H), 4.33 (t, J=8.0 Hz, 2H), 3.56 (q, J=8.0 Hz, 2H), 2.64 (s, 2H), 2.25-2.17 (m, 1H), 1.09-1.08 (m, 2H), 0.96 (s, 9H), 0.88-0.85 (m, 2H)

### Example 402: N-(2-(5-(4-methyl-2-(trifluoromethyl)phenyl)-3-neopentyl-1H-indol-1-yl)ethyl)cyclopropanesulfonamide (402)

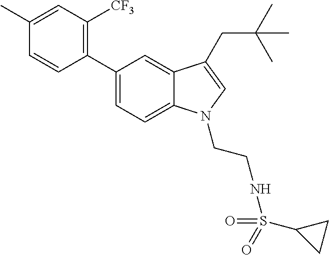 [SMILES: Cc1ccc(-c2ccc3c(c2)c(CC(C)(C)C)cn3CCNS(=O)(=O)C2CC2)c(C(F)(F)F)c1]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 1-bromo-4-methyl-2-(trifluoromethyl)benzene. ¹HNMR (CDCl₃, 400 MHz) δ 7.51 (s, 1H), 7.50 (s, 1H), 7.35-7.24 (m, 3H), 7.14 (d, J=8.0 Hz, 1H), 6.94 (d, J=4.0 Hz, 1H), 4.37-4.30 (m, 3H), 3.61-3.54 (m, 2H), 2.64 (s, 2H), 2.46 (s, 3H), 2.20-2.13 (m, 1H), 1.07-1.04 (m, 2H), 0.93 (s, 9H), 0.84-0.80 (m, 2H).

### Example 403: N-[(1S)-2,2,2-trifluoro-1-[5-fluoro-1-(3-methoxy-2,2-dimethyl-propyl)-6-[2-(trifluoromethyl)phenyl]indol-3-yl]ethyl]cyclopropanesulfonamide (403)

### Step 1: 6-bromo-5-fluoro-1-(3-methoxy-2,2-dimethyl-propyl)indole

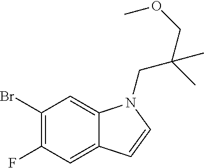 [SMILES: COCC(C)(C)Cn1ccc2cc(F)c(Br)cc21]

To a stirred solution of 3-(6-bromo-5-fluoro-indol-1-yl)-2,2-dimethyl-propan-1-ol (0.9 g, 3.00 mmol) in THF (10 mL) was added HNa (479.69 mg, 11.99 mmol, 60% purity) at 0° C. under N₂. The mixture was stirred at rt for 30 min. iodomethane (1.70 g, 11.99 mmol, 746.63 uL) was injected to the mixture. The reaction was stirred at RT for 2 h. TLC showed a new spot and start material was consumed. The reaction was quenched by water and extracted with EA (100 mL×2). The combined organic layers were dried over anhydrous Na₂SO₄, filtered and concentrated. The residue obtained was purified by column flash of silica gel eluting with PE/EA from 5% to 15% to give 6-bromo-5-fluoro-1-(3-methoxy-2,2-dimethyl-propyl)indole (0.9 g, 2.86 mmol, 95.54% yield) as a yellow oil.

### Step 2: (S)—N-[(1S)-1-[6-bromo-5-fluoro-1-(3-methoxy-2,2-dimethyl-propyl)indol-3-yl]-2,2,2-trifluoro-ethyl]-2-methyl-propane-2-sulfinamide

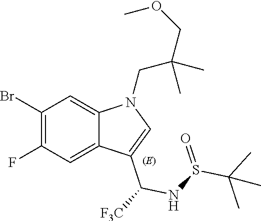 [SMILES: COCC(C)(C)Cn1cc([C@H](N[S@@](=O)C(C)(C)C)C(F)(F)F)c2cc(F)c(Br)cc21]

The mixture of (NE,S)-2-methyl-N-(2,2,2-trifluoroethylidene)propane-2-sulfinamide (2.02 g, 10.03 mmol) in DCM (10 mL) was cooled to −20° C., BF₃·Et₂O (1.22 g, 8.59 mmol, 1.06 mL) was added slowly, followed by addition DCM solution of 6-bromo-5-fluoro-1-(3-methoxy-2,2-dimethyl-propyl)indole (0.9 g, 2.86 mmol). The mixture was then continued to stir at −10° C. for 1.5 h. The mixture was diluted with DCM, followed by water. The aqueous layer was extracted with DCM twice. Organic layers were combined and washed with brine and concentrated in vacuo to give (S)—N-[(1S)-1-[6-bromo-5-fluoro-1-(3-methoxy-2,2-dimethyl-propyl)indol-3-yl]-2,2,2-trifluoro-ethyl]-2-methyl-propane-2-sulfinamide (2.1 g, 4.07 mmol, 142.24% yield).

### Step 3: The synthesis of (1S)-1-[6-bromo-5-fluoro-1-(3-methoxy-2,2-dimethyl-propyl)indol-3-yl]-2,2,2-trifluoro-ethanamine

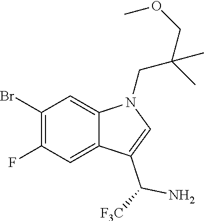 [SMILES: COCC(C)(C)Cn1cc([C@H](N)C(F)(F)F)c2cc(F)c(Br)cc21]

To a (S)—N-[(1S)-1-[6-bromo-5-fluoro-1-(3-methoxy-2,2-dimethyl-propyl)indol-3-yl]-2,2,2-trifluoro-ethyl]-2-methyl-propane-2-sulfinamide (2.0 g, 3.88 mmol) was added 4N HCl/MeOH (15 mL). The mixture was stirred at RT for 1.5 h. The reaction was concentrated and adjusted pH=8 by addition aq. NaHCO₃. The aqueous layer was extracted by DCM (50 mL×2). The combined organic layers were dried over anhydrous Na₂SO₄, filtered and concentrated. The residue obtained was purified by column chromatography of silica gel eluting with PE/EA from 50/1 to 10/1 to give (1S)-1-[6-bromo-5-fluoro-1-(3-methoxy-2,2-dimethyl-propyl)indol-3-yl]-2,2,2-trifluoro-ethanamine (0.95 g, 2.31 mmol, 59.53% yield) as a brown oil.

### Step 4: (1S)-2,2,2-trifluoro-1-[5-fluoro-1-(3-methoxy-2,2-dimethyl-propyl)-6-[2-(trifluoromethyl)phenyl]indol-3-yl]ethanamine

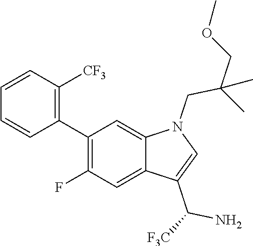 [SMILES: COCC(C)(C)Cn1cc([C@H](N)C(F)(F)F)c2cc(F)c(-c3ccccc3C(F)(F)F)cc21]

A mixture of (1S)-1-[6-bromo-5-fluoro-1-(3-methoxy-2,2-dimethyl-propyl)indol-3-yl]-2,2,2-trifluoro-ethanamine (500 mg, 1.22 mmol), [2-(trifluoromethyl)phenyl]boronic acid (300.20 mg, 1.58 mmol), cyclopentyl (diphenyl)phosphane;dichloropalladium;iron (266.89 mg, 364.76 umol) and disodium;carbonate (386.60 mg, 3.65 mmol, 152.81 uL) in DIOXANE (5 mL) and H2O (1 mL) was stirred at 80° C. for 2 h under Nitrogen. The mixture was extracted with EA (50 mL×2). The combined organic layers were concentrated, the residue was purified by silica gel column (EA/PE=0-50%) to get (1S)-2,2,2-trifluoro-1-[5-fluoro-1-(3-methoxy-2,2-dimethyl-propyl)-6-[2-(trifluoromethyl)phenyl]indol-3-yl]ethanamine (400 mg, 839.58 umol, 69.05% yield) as a yellow oil.

### Step 6: N-[(1S)-2,2,2-trifluoro-1-[5-fluoro-1-(3-methoxy-2,2-dimethyl-propyl)-6-[2-(trifluoromethyl)phenyl]indol-3-yl]ethyl]cyclopropanesulfonamide

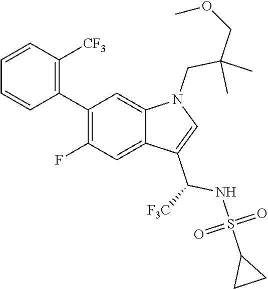 [SMILES: COCC(C)(C)Cn1cc([C@H](NS(=O)(=O)C2CC2)C(F)(F)F)c2cc(F)c(-c3ccccc3C(F)(F)F)cc21]

To a mixture of (1S)-2,2,2-trifluoro-1-[5-fluoro-1-(3-methoxy-2,2-dimethyl-propyl)-6-[2-(trifluoromethyl)phenyl]indol-3-yl]ethanamine (0.2 g, 419.79 umol) in anhydrous pyridine (5 mL) at RT was added cyclopropanesulfonyl chloride (177.05 mg, 1.26 mmol, 128.30 uL). The mixture was stirred at RT overnight and the completion of reaction was monitored by lcms. The mixture was concentrated and diluted with EtOAc, washed with 2N HCl, water, saturated NaHCO₃ and brine and dried over Na₂SO₄. Solvent was removed in vacuo to obtain the crude which was purified by chromatography on silica gel (EtOAc/hex) to afford N-[(1S)-2,2,2-trifluoro-1-[5-fluoro-1-(3-methoxy-2,2-dimethyl-propyl)-6-[2-(trifluoromethyl)phenyl]indol-3-yl]ethyl]cyclopropanesulfonamide (82 mg, 141.24 umol, 33.65% yield) as a yellow solid. ¹H NMR (400 MHz, MeOD) δ 7.80 (d, J=7.7 Hz, 1H), 7.66 (t, J=7.2 Hz, 1H), 7.58 (t, J=7.6 Hz, 1H), 7.52 (d, J=9.7 Hz, 1H), 7.48 (s, 1H), 7.42 (d, J=7.5 Hz, 1H), 7.36 (d, J=6.0 Hz, 1H), 5.41 (q, J=7.9 Hz, 1H), 4.06 (d, J=2.4 Hz, 2H), 3.26 (s, 3H), 3.05-2.92 (m, 2H), 2.39-2.22 (m, 1H), 1.09-0.97 (m, 2H), 0.93 (dd, J=15.3, 8.9 Hz, 6H), 0.83-0.68 (m, 2H). LCMS: 580.8 m/z [M+1]⁺.

### Example 404: N-[(1S)-1-[1-[2-(chloromethyl)-3-hydroxy-2-methyl-propyl]-5-fluoro-6-[2-(trifluoromethyl)phenyl]indol-3-yl]-2,2,2-trifluoro-ethyl]cyclopropanesulfonamide (404)

### Step 1: 6-bromo-5-fluoro-1-[(3-methyloxetan-3-yl)methyl]indole

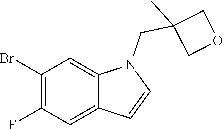 [SMILES: CC1(Cn2ccc3cc(F)c(Br)cc32)COC1]

6-bromo-5-fluoro-1H-indole (1 g, 4.67 mmol) was added to a suspension of NaH (275 mg, 6.88 mmol, 60% purity) in anhydrous DMF (8 mL) in ice-water under nitrogen. The mixture was stirred for 1 h at room temperature, then cooling in ice-water for the addition of 3-(bromomethyl)-3-methyl-oxetane (1.00 g, 6.07 mmol) and the resulting mixture was stirred at room temperature overnight under nitrogen. LCMS showed the starting material was consumed completely. After quenching with water, the aqueous phase was extracted with ethyl acetate three times. The combined organic layer was wash with sat. NH₄Cl twice and brine, dried over anhydrous sodium sulfate. After the solvent was removed in vacuo, flash chromatography (EA/PE=0-50%) yielded 6-bromo-5-fluoro-1-[(3-methyloxetan-3-yl)methyl]indole (1.37 g, 4.59 mmol) as a yellow solid.

### Step 2: (S)—N-[(1S)-1-[6-bromo-5-fluoro-1-[(3-methyloxetan-3-yl)methyl]indol-3-yl]-2,2,2-trifluoro-ethyl]-2-methyl-propane-2-sulfinamide

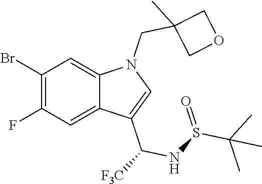

The mixture of 6-bromo-5-fluoro-1-[(3-methyloxetan-3-yl)methyl]indole (500 mg, 1.68 mmol) in anhydrous CH₂Cl₂ (10 mL) was cooled to −10° C., BF₃·OEt₂ (476.88 mg, 3.36 mmol, 414.68 uL) was added slowly, followed by addition of a solution of (S,E)-2-methyl-N-(2,2,2-trifluoroethylidene)propane-2-sulfinamide in DCM. The mixture was then continued to stir for 1-2 h. The mixture was diluted with DCM, followed by water. The aqueous layer was extracted with DCM twice. Organic layers were combined and washed with brine and concentrated in vacuo to obtain the crude (S)—N-[(1S)-1-[6-bromo-5-fluoro-1-[(3-methyloxetan-3-yl)methyl]indol-3-yl]-2,2,2-trifluoro-ethyl]-2-methyl-propane-2-sulfinamide (460 mg, 921.18 μmol) as yellow oil.

### Step 3: 3-(3-((S)-1-amino-2,2,2-trifluoroethyl)-6-bromo-5-fluoro-1H-indol-1-yl)-2-(chloromethyl)-2-methylpropan-1-ol

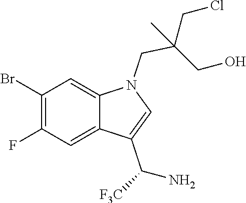 [SMILES: CC(CO)(CCl)Cn1cc([C@H](N)C(F)(F)F)c2cc(F)c(Br)cc21]

A mixture of (S)—N-[(1S)-1-[6-bromo-5-fluoro-1-[(3-methyloxetan-3-yl)methyl]indol-3-yl]-2,2,2-trifluoro-ethyl]-2-methyl-propane-2-sulfinamide (720 mg, 1.44 mmol) in 4M HCl(g)/MeOH (10 mL) was stirred at room temperature for 3 hours. The mixture was concentrated in vacuo, the residue was basified to pH=8 with sat. NaHCO₃, the aqueous phase was extracted with EtOAc twice. The combined organic phases were washed with brine, dried with anhydrous Na2SO4 and filtered. The filtrate was concentrated to give the crude product, which was purified by prep-HPLC to get 3-(3-((S)-1-amino-2,2,2-trifluoroethyl)-6-bromo-5-fluoro-1H-indol-1-yl)-2-(chloromethyl)-2-methylpropan-1-ol (520 mg, 1.20 mmol, 83.66%) as colorless oil.

### Step 4: N-((1S)-1-(6-bromo-1-(3-chloro-2-(hydroxymethyl)-2-methylpropyl)-5-fluoro-1H-indol-3-yl)-2,2,2-trifluoroethyl)cyclopropanesulfonamide

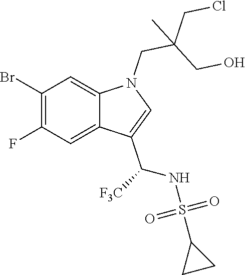 [SMILES: CC(CO)(CCl)Cn1cc([C@H](NS(=O)(=O)C2CC2)C(F)(F)F)c2cc(F)c(Br)cc21]

A mixture of 3-(3-((S)-1-amino-2,2,2-trifluoroethyl)-6-bromo-5-fluoro-1H-indol-1-yl)-2-(chloromethyl)-2-methylpropan-1-ol (220 mg, 532.42 μmol) and cyclopropanesulfonyl chloride (193.95 mg, 1.38 mmol, 140.55 uL) in pyridine (1.5 mL) was stirred at RT overnight. The mixture was acidified to pH=1 with 1N HCl, then was extracted with EtOAc twice. The combined organic phases were washed with brine, dried with Na₂SO₄, filtered and concentrated to give the crude product, which was purified by silica gel column (EA/PE=0-80%) to get N-((1S)-1-(6-bromo-1-(3-chloro-2-(hydroxymethyl)-2-methylpropyl)-5-fluoro-1H-indol-3-yl)-2,2,2-trifluoroethyl)cyclopropanesulfonamide (187 mg, 361.47 μmol, 65.55% yield) as yellow oil.

### Step 5: N-[(1S)-1-[1-[2-(chloromethyl)-3-hydroxy-2-methyl-propyl]-5-fluoro-6-[2-(trifluoromethyl)phenyl]indol-3-yl]-2,2,2-trifluoro-ethyl]cyclopropanesulfonamide

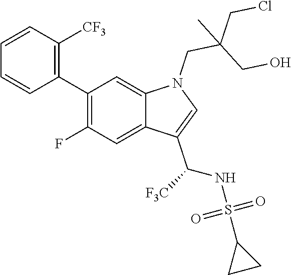 [SMILES: CC(CO)(CCl)Cn1cc([C@H](NS(=O)(=O)C2CC2)C(F)(F)F)c2cc(F)c(-c3ccccc3C(F)(F)F)cc21]

A mixture of N-[(1S)-1-[6-bromo-1-[2-(chloromethyl)-3-hydroxy-2-methyl-propyl]-5-fluoro-indol-3-yl]-2,2,2-trifluoro-ethyl]cyclopropanesulfonamide (187 mg, 349.03 μmol), [2-(trifluoromethyl)phenyl]boronic acid (99.43 mg, 523.54 μmol) and K2CO3 (144.71 mg, 1.05 mmol) in dioxane (4 mL) and water (1.5 mL) was stirred at 80° C. overnight under Nitrogen. The mixture was added water and EtOAc, the mixture was stirred and separated, the aqueous phase was extracted with EtOAc twice, the combined organic phases were washed with brine and dried with Na2SO4, filtered through silica gel to get oil, which was purified by prep-HPLC to get N-[(1S)-1-[1-[2-(chloromethyl)-3-hydroxy-2-methyl-propyl]-5-fluoro-6-[2-(trifluoromethyl)phenyl]indol-3-yl]-2,2,2-trifluoro-ethyl]cyclopropanesulfonamide (86.21 mg, 143.45 μmol) as a white solid. ¹H NMR (400 MHz, DMSO) δ 8.61 (s, 1H), 7.86 (d, J=7.7 Hz, 1H), 7.80-7.70 (m, 3H), 7.66 (t, J=7.6 Hz, 1H), 7.61-7.53 (m, 1H), 7.48 (d, J=7.5 Hz, 1H), 5.52 (q, J=8.0 Hz, 1H), 5.00 (dd, J=12.1, 6.7 Hz, 1H), 4.15 (dd, J=23.1, 12.5 Hz, 2H), 3.67-3.48 (m, 2H), 3.30-3.14 (m, 2H), 2.41-2.31 (m, 1H), 0.92 (d, J=3.8 Hz, 2H), 0.80 (dd, J=8.2, 5.0 Hz, 3H), 0.77-0.59 (m, 2H). LCMS: 600.7 [M+H]⁺ and 617.8 m/z [M+H₂O]⁺.

### Example 405: (S)—N-(1-(1-(3-cyano-2,2-dimethylpropyl)-5-fluoro-6-(2-(trifluoromethyl)phenyl)-1H-indol-3-yl)-2,2,2-trifluoroethyl)cyclopropanesulfonamide (405)

### Step 1: 3-(6-bromo-5-fluoro-indol-1-yl)-2,2-dimethyl-propan-1-ol

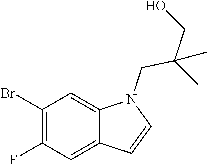 [SMILES: CC(C)(CO)Cn1ccc2cc(F)c(Br)cc21]

To a stirred solution of 6-bromo-5-fluoro-1H-indole (1.5 g, 7.01 mmol) and 1,4,7,10,13,16-hexaoxacyclooctadecane (2.41 g, 9.11 mmol, 2.04 mL) in anhydrous THF (20 mL) was added NaH (701.13 mg, 17.53 mmol, 60% purity) at 5° C. Then the mixture was heated to 80° C. To the reaction mixture was added a solution for 5,5-dimethyl-1,3,2-dioxathiane 2,2-dioxide (874.19 mg, 5.26 mmol) in THF (5 ml), and the stirring was continued at 80° C. overnight. The reaction mixture was cooled down to 0° C., and conc. HCl was added slowly until pH reached 1-2. Then the reaction mixture was further stirred at 80° C. for 2 hours. After neutralized with saturated aqueous NaHCO3 solution, the reaction mixture was partitioned between ethyl acetate and water. The organic layer was washed with brine, dried with anhydrous Na₂SO₄, filtered, and evaporated. The residue was purified by silica gel column (EA/PE=0-33%) to get 3-(6-bromo-5-fluoro-indol-1-yl)-2,2-dimethyl-propan-1-ol (1.82 g, 6.05 mmol) as yellow oil.

### Step 2: [3-(6-bromo-5-fluoro-indol-1-yl)-2,2-dimethyl-propyl]methanesulfonate

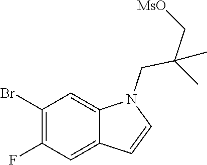 [SMILES: CC(C)(COS(C)(=O)=O)Cn1ccc2cc(F)c(Br)cc21]

To a stirred solution of 3-(6-bromo-5-fluoro-indol-1-yl)-2,2-dimethyl-propan-1-ol (1.82 g, 6.06 mmol) and Triethylamine (2.45 g, 24.25 mmol, 3.38 mL) in anhydrous CH2Cl2 (50 mL) was added dropwise methanesulfonyl chloride (1.39 g, 12.13 mmol, 938.59 uL) at 0° C., the mixture was stirred at 0-25° C. for 2 hours under N2, then to the mixture was added water. The organic phase was separated, the organic was extracted with CH₂Cl₂, the combined organic phases were washed with brine, dried with anhydrous Na₂SO₄ and filtered. The filtrate was concentrated to get yellow oil, which was purified by silica gel column (EA/PE=10-33%) to get [3-(6-bromo-5-fluoro-indol-1-yl)-2,2-dimethyl-propyl]methanesulfonate (2.09 g, 5.53 mmol, 91.13% yield) as light yellow oil.

### Step 3: 4-(6-bromo-5-fluoro-indol-1-yl)-3,3-dimethyl-butanenitrile

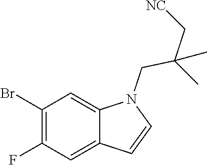 [SMILES: CC(C)(CC#N)Cn1ccc2cc(F)c(Br)cc21]

A mixture of [3-(6-bromo-5-fluoro-indol-1-yl)-2,2-dimethyl-propyl]methanesulfonate (1.99 g, 5.26 mmol), trimethylsilylformonitrile (2.16 g, 21.77 mmol) and K₂CO₃ (3.18 g, 23.01 mmol) in anhydrous DMF (9 mL) was stirred at 120° C. for 4 hours and 6 hours in the microwave twice. The mixture was cooled to rt and added EtOAc, the black mixture was filtered through diatomite, to the filtrate was added water and stirred, the organic phase was separated, the aqueous phase was extracted with EtOAc twice, the combine organic phases were washed with brine, dried with anhydrous Na₂SO₄ and filtered. The filtrate was concentrated to give the crude product, which was purified by prep-HPLC to get 4-(6-bromo-5-fluoro-indol-1-yl)-3,3-dimethyl-butanenitrile (400 mg, 1.29 mmol).

### Step 4: (S)—N—((S)-1-(6-bromo-1-(3-cyano-2,2-dimethylpropyl)-5-fluoro-1H-indol-3-yl)-2,2,2-trifluoroethyl)-2-methylpropane-2-sulfinamide

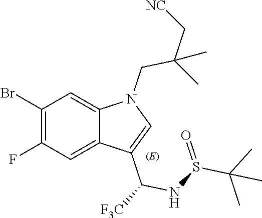 [SMILES: CC(C)(CC#N)Cn1cc([C@H](N[S@@](=O)C(C)(C)C)C(F)(F)F)c2cc(F)c(Br)cc21]

The mixture of 4-(6-bromo-5-fluoro-indol-1-yl)-3,3-dimethyl-butanenitrile (400 mg, 1.29 mmol) in anhydrous CH₂Cl₂ (8 mL) was cooled to −10° C., BF3·OEt2 (549.27 mg, 3.87 mmol, 477.63 uL) was added slowly, followed by addition of a solution of (S,E)-2-methyl-N-(2,2,2-trifluoroethylidene)propane-2-sulfinamide in DCM. The resulting mixture was allowed to raise to r.t. and stirred for 3 h. The mixture was diluted with DCM, followed by water. The aqueous layer was extracted with DCM twice. Organic layers were combined and washed with brine and concentrated in vacuo to obtain the crude product as brown oil, which was used directly next step.

### Step 5: 4-[3-[(1S)-1-amino-2,2,2-trifluoro-ethyl]-6-bromo-5-fluoro-indol-1-yl]-3,3-dimethyl-butanenitrile

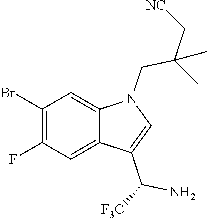 [SMILES: CC(C)(CC#N)Cn1cc([C@H](N)C(F)(F)F)c2cc(F)c(Br)cc21]

A mixture of (S)—N-[(1S)-1-[6-bromo-1-(3-cyano-2,2-dimethyl-propyl)-5-fluoro-indol-3-yl]-2,2,2-trifluoro-ethyl]-2-methyl-propane-2-sulfinamide (658.40 mg, 1.29 mmol) in 4M HCl(g)/MeOH (30 mL) was stirred at RT for 2 hours, the mixture was concentrated, the residue was basified to pH=8 with sat. NaHCO₃, the aqueous phase was extracted with EtOAc twice, the combined organic phases were washed with brine, dried with anhydrous Na2SO4 and filtered. The filtrate was concentrated to give the crude product, which was purified by silica gel column (EA/PE=0-50%) to get 4-[3-[(1S)-1-amino-2,2,2-trifluoro-ethyl]-6-bromo-5-fluoro-indol-1-yl]-3,3-dimethyl-butanenitrile (513 mg, 1.26 mmol, 97.90% yield) as light yellow oil.

### Step 7: (S)-4-(3-(1-amino-2,2,2-trifluoroethyl)-5-fluoro-6-(2-(trifluoromethyl)phenyl)-1H-indol-1-yl)-3,3-dimethylbutanenitrile

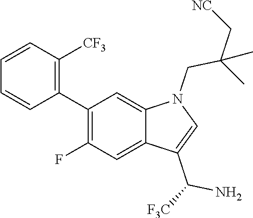 [SMILES: CC(C)(CC#N)Cn1cc([C@H](N)C(F)(F)F)c2cc(F)c(-c3ccccc3C(F)(F)F)cc21]

A mixture of 4-[3-[(1S)-1-amino-2,2,2-trifluoro-ethyl]-6-bromo-5-fluoro-indol-1-yl]-3,3-dimethyl-butanenitrile (150 mg, 369.26 μmol), [2-(trifluoromethyl)phenyl]boronic acid (105.20 mg, 553.89 μmol), Pd(PPh₃)₄ (128.01 mg, 110.78 μmol) and Na₂CO₃ (136.98 mg, 1.29 mmol) in dioxane (3 mL) and water (0.75 mL) was stirred at 80° C. for 4 hours under Nitrogen. To the mixture was added water and EtOAc, the mixture was stirred and separated, the aqueous phase was extracted with EtOAc twice, the combined organic phases were washed with brine, dried with anhydrous Na₂SO₄ and filtered through silica gel. The filtrate was concentrated to get brown oil, which was used directly next step.

### Step 8: (S)—N-(1-(1-(3-cyano-2,2-dimethylpropyl)-5-fluoro-6-(2-(trifluoromethyl)phenyl)-1H-indol-3-yl)-2,2,2-trifluoroethyl)cyclopropanesulfonamide

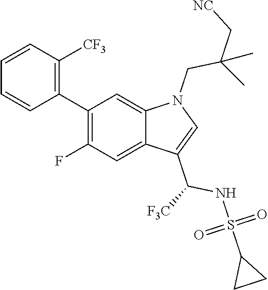 [SMILES: CC(C)(CC#N)Cn1cc([C@H](NS(=O)(=O)C2CC2)C(F)(F)F)c2cc(F)c(-c3ccccc3C(F)(F)F)cc21]

A mixture of (S)-4-(3-(1-amino-2,2,2-trifluoroethyl)-5-fluoro-6-(2-(trifluoromethyl)phenyl)-1H-indol-1-yl)-3,3-dimethylbutanenitrile (158.56 mg, 336.35 μmol) and cyclopropanesulfonyl chloride (259.53 mg, 1.85 mmol, 188.06 uL) in pyridine (2.5 mL) was stirred at RT overnight. The mixture was concentrated for remove pyridine to get brown oil, which was purified by silica gel column (EA/PE=0-50%) to get crude product as brown oil, which was purified by prep-HPLC to get (S)—N-(1-(1-(3-cyano-2,2-dimethylpropyl)-5-fluoro-6-(2-(trifluoromethyl)phenyl)-1H-indol-3-yl)-2,2,2-trifluoroethyl)cyclopropanesulfonamide (27.55 mg, 47.87 μmol) as a white solid. ¹H NMR (400 MHz, CDCl₃) δ 7.79 (d, J=7.5 Hz, 1H), 7.60 (t, J=7.4 Hz, 1H), 7.52 (dd, J=16.2, 8.6 Hz, 2H), 7.37 (d, J=7.4 Hz, 1H), 7.30 (s, 1H), 7.23 (d, J=5.8 Hz, 1H), 5.39 (p, J=7.6 Hz, 1H), 5.02 (d, J=29.7 Hz, 1H), 4.03 (s, 2H), 2.50 (s, 1H), 2.27 (s, 2H), 1.34-1.20 (m, 2H), 1.15 (s, 6H), 1.01 (dd, J=13.0, 5.5 Hz, 2H). LCMS: 592.8 m/z [M+H]⁺.

### Example 406: N-(1-((1-neopentyl-6-(2-(trifluoromethyl)phenyl)-1H-indol-3-yl)methyl)cyclopropyl)cyclopropanesulfonamide (406)

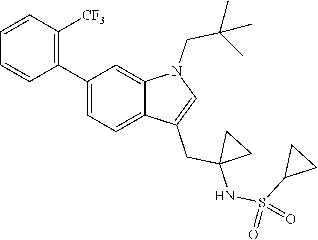 [SMILES: CC(C)(C)Cn1cc(CC2(NS(=O)(=O)C3CC3)CC2)c2ccc(-c3ccccc3C(F)(F)F)cc21]

### Step 1: 2-(6-bromo-1H-indol-3-yl)acetonitrile

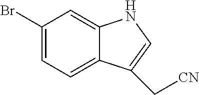 [SMILES: N#CCc1c[nH]c2cc(Br)ccc12]

The title compound was prepared following the same general protocol as described for Step 3, Example 98, using 6-bromo-1H-indole. ESI-MS (m/z): 234.45, 236.64 [M+1]⁺.

### Step 2: 6-bromo-7-fluoro-1-neopentyl-1H-indole

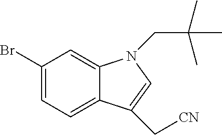 [SMILES: CC(C)(C)Cn1cc(CC#N)c2ccc(Br)cc21]

The title compound was prepared following the same general protocol as described for Example 34, using 2-(6-bromo-1H-indol-3-yl)acetonitrile and 1-iodo-2,2-dimethylpropane. ¹HNMR (CDCl₃, 400 MHz) δ 7.50 (d, J=4.0 Hz, 1H), 7.41 (d, J=8.0 Hz, 1H), 7.24 (d, J=8.0 Hz, 1H), 7.06 (s, 1H), 3.82 (s, 2H), 3.80 (s, 2H), 1.00 (s, 9H)

### Step 3: 1-((6-bromo-1-neopentyl-1H-indol-3-yl)methyl)cyclopropan-1-amine

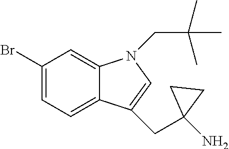 [SMILES: CC(C)(C)Cn1cc(CC2(N)CC2)c2ccc(Br)cc21]

To a mixture of 6-bromo-7-fluoro-1-neopentyl-1H-indole (0.526 g, 1.72 mmol) in THF was added TiMe(Oi-Pr)₃ (2.6 mL, 1.0 M in THF, 2.6 mmol), then ethylmagnesium bromide (0.87 mL, 3.0M in ether, 2.6 mmol) was added dropwise. The mixture was stirred for 1.5 h at rt, followed by addition of BF₃·OEt₂ (0.43 mL, 3.44 mmol). The mixture stirred for another 30 min, and then quenched by addition of 1N HCl (17 mL). EtOAc was added to dilute the mixture, 3M NaOH was used to adjust the pH-9. EtOAc was applied to extract the aqueous layer 2 times. The organic layers were combined and washed with brine and dried over Na₂SO₄. The solvent was concentrated and the crude was purified via silica gel to obtain the title compound. ESI-MS (m/z): 334.8, 336.45 [M+1]⁺.

### Step 4: N-(1-((6-bromo-1-neopentyl-1H-indol-3-yl)methyl)cyclopropyl)cyclopropanesulfonamide

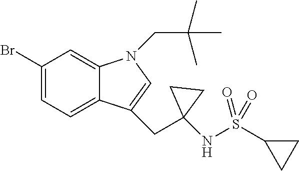 [SMILES: CC(C)(C)Cn1cc(CC2(NS(=O)(=O)C3CC3)CC2)c2ccc(Br)cc21]

The title compound was prepared following the same general protocol as described for Step 5, Example 116, using N-(1-((6-bromo-1-neopentyl-1H-indol-3-yl)methyl)cyclopropyl)cyclopropanesulfonamide. ¹HNMR (CDCl₃, 400 MHz) δ 7.47 (d, J=4.0 Hz, 1H), 7.44 (d, J=8.0 Hz, 1H), 7.18 (dd, J=8.0 Hz, 4.0 Hz, 1H), 7.03 (s, 1H), 4.62 (s, 1H), 3.83 (s, 2H), 3.09 (s, 2H), 2.40-2.33 (m, 1H), 1.21-1.20 (m, 2H), 1.06-1.03 (m, 2H), 1.00-0.97 (m, 11H), 0.83 (t, J=8.0 Hz, 2H)

### Step 5: N-(1-((1-neopentyl-6-(2-(trifluoromethyl)phenyl)-1H-indol-3-yl)methyl)cyclopropyl)cyclopropanesulfonamide

The title compound was prepared following the same general protocol as described for Step 5, Example 1, using N-(1-((6-bromo-1-neopentyl-1H-indol-3-yl)methyl)cyclopropyl)cyclopropanesulfonamide and (2-(trifluoromethyl)phenyl)boronic acid. ¹HNMR (CDCl₃, 400 MHz) δ 7.75 (d, J=8.0 Hz, 1H), 7.58 (d, J=8.0 Hz, 1H), 7.55 (d, J=8.0 Hz, 1H), 7.46 (t, J=8.0 Hz, 1H), 7.39 (d, J=8.0 Hz, 1H), 7.29 (s, 1H), 7.11 (s, 1H), 7.04 (d, J=8.0 Hz, 1H), 4.74 (s, 1H), 3.88 (s, 2H), 3.14 (s, 2H), 2.42-2.36 (m, 1H), 1.25-1.20 (m, 2H), 1.12-1.09 (m, 2H), 1.03-0.97 (m, 11H), 0.83 (m, 2H)

### Example 407: 1-isobutyl-3-(1,1,1-trifluoro-3-(methylsulfonyl)propan-2-yl)-6-(2-(trifluoromethyl)phenyl)-1H-indole (407)

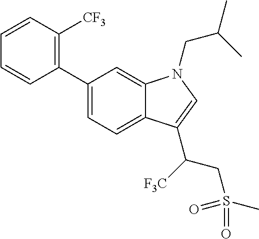 [SMILES: CC(C)Cn1cc(C(CS(C)(=O)=O)C(F)(F)F)c2ccc(-c3ccccc3C(F)(F)F)cc21]

### Step 1: 1-(6-bromo-1-isobutyl-1H-indol-3-yl)-2,2,2-trifluoroethan-1-one

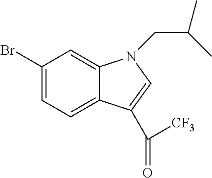 [SMILES: CC(C)Cn1cc(C(=O)C(F)(F)F)c2ccc(Br)cc21]

The title compound was prepared following the same general protocol as described for Step 1, Example 1, using 1-(6-bromo-1H-indol-3-yl)-2,2,2-trifluoroethan-1-one. ESI-MS (m/z): 347.53, 349.61 [M+1]⁺.

### Step 2: 2,2,2-trifluoro-1-(1-isobutyl-6-(2-(trifluoromethyl)phenyl)-1H-indol-3-yl)ethan-1-one

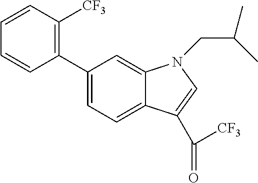 [SMILES: CC(C)Cn1cc(C(=O)C(F)(F)F)c2ccc(-c3ccccc3C(F)(F)F)cc21]

The title compound was prepared following the same general protocol as described for Step 5, Example 1, using 1-(6-bromo-1-isobutyl-1H-indol-3-yl)-2,2,2-trifluoroethan-1-one and (2-(trifluoromethyl)phenyl)boronic acid. ESI-MS (m/z): 413.78 [M+1]⁺.

### Step 3: 1-isobutyl-3-(3,3,3-trifluoro-1-(methylsulfonyl)prop-1-en-2-yl)-6-(2-(trifluoromethyl)phenyl)-1H-indole

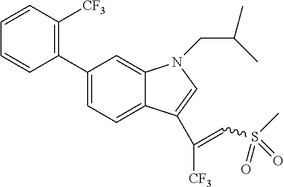 [SMILES: CC(C)Cn1cc(C(=CS(C)(=O)=O)C(F)(F)F)c2ccc(-c3ccccc3C(F)(F)F)cc21]

To a mixture of diethyl ((methylsulfonyl)methyl)phosphonate (0.184 g, 0.795 mmol) in THF (2 mL) at −78° C. under argon was added LiHMDS (0.85 mL, 1M in THF, 0.85 mmol) dropwise. The mixture was continued to stir for 30 min, 2,2,2-trifluoro-1-(1-isobutyl-6-(2-(trifluoromethyl)phenyl)-1H-indol-3-yl)ethan-1-one (0.22 g, 0.53 mmol) in THF (0.5 mL) was added. The reaction was monitored by anal. HPLC for completion. The mixture was quenched by addition of saturated NH₄Cl and diluted with EtOAc. The layers were separated, and the aqueous layer was extracted with EtOAc (2×). The combined organics were concentrated and the crude residue was purified by silica gel to obtain the title compound. ¹HNMR (CDCl₃, 400 MHz) δ 7.83 (d, J=8.0 Hz, 1H), 7.78 (d, J=8.0 Hz, 1H), 7.60 (t, J=8.0 Hz, 1H), 7.54-7.49 (m, 2H), 7.40 (d, J=8.0 Hz, 1H), 7.36 (s, 1H), 7.25 (d, J=8.0 Hz, 1H), 7.11 (s, 1H), 3.92 (d, J=8.0 Hz, 2H), 3.24 (s, 3H), 2.40-2.17 (m, 1H), 1.25-1.20 (m, 2H), 0.96 (d, J=8.0 Hz, 6H), 0.88-0.78 (m, 2H)

### Step 4: 1-isobutyl-3-(1,1,1-trifluoro-3-(methylsulfonyl)propan-2-yl)-6-(2-(trifluoromethyl)phenyl)-1H-indole

To a solution of 1-isobutyl-3-(3,3,3-trifluoro-1-(methylsulfonyl)prop-1-en-2-yl)-6-(2-(trifluoromethyl)phenyl)-1H-indole (0.204 g) in EtOH (1 mL) was added Pd/C (0.02 g) and the reaction mixture was stirred under a hydrogen balloon at rt overnight. The mixture was filtered through a pad of celite and concentrated, the crude was purified by silica gel to obtain the title compound. ¹HNMR (CDCl₃, 400 MHz) δ 7.76 (d, J=8.0 Hz, 1H), 7.66 (d, J=8.0 Hz, 1H), 7.58 (t, J=8.0 Hz, 1H), 7.48 (t, J=8.0 Hz, 1H), 7.41 (d, J=8.0 Hz, 1H), 7.32 (s, 1H), 7.22 (s, 1H), 7.15 (d, J=8.0 Hz, 1H), 4.30 (q, J=8.0 Hz, 1H), 3.93 (d, J=8.0 Hz, 2H), 3.85-3.49 (m, 2H), 2.35 (s, 3H), 2.20-2.13 (m, 1H), 1.25-1.20 (m, 2H), 0.96 (m, 8H)

### Example 408: 3-(2-(cyclopropylsulfonyl)ethyl)-6-(4-methyl-2-(trifluoromethyl)phenyl)-1-neopentyl-1H-indole (408)

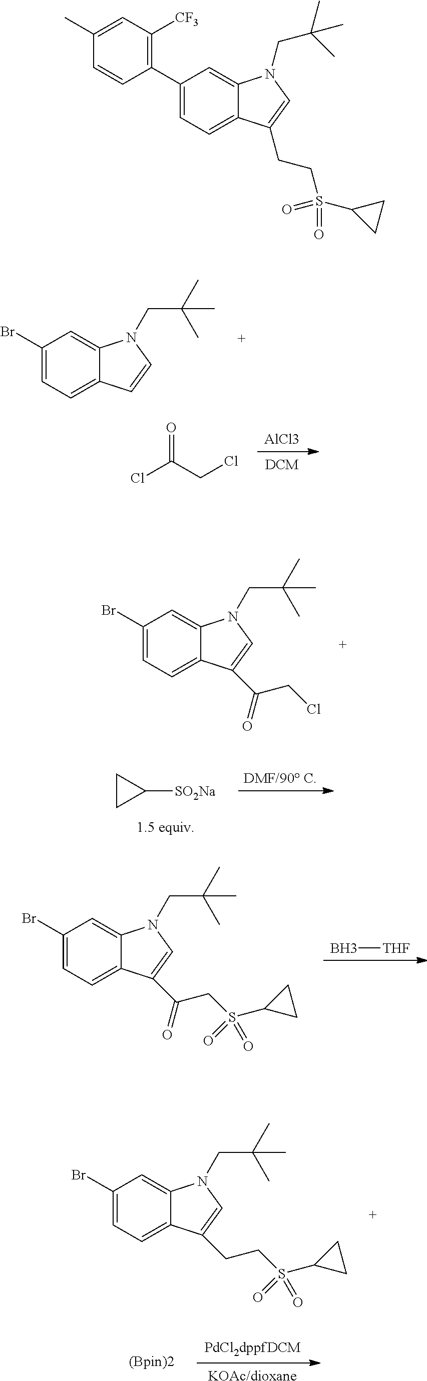

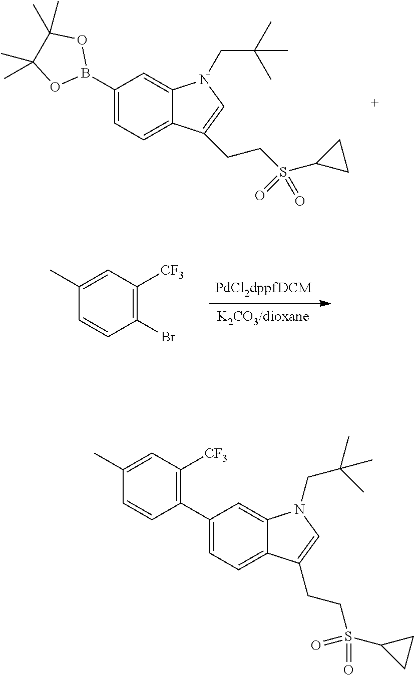 [SMILES: CC(C)(C)Cn1cc(CCS(=O)(=O)C2CC2)c2ccc(B3OC(C)(C)C(C)(C)O3)cc21 ; Cc1ccc(Br)c(C(F)(F)F)c1 ; "K2CO3/dioxane" ; "PdCl2dppfDCM" ; Cc1ccc(-c2ccc3c(CCS(=O)(=O)C4CC4)cn(CC(C)(C)C)c3c2)c(C(F)(F)F)c1]

### Step 1: 1-(6-bromo-1-neopentyl-1H-indol-3-yl)-2-chloroethan-1-one

The title compound was prepared following the same general protocol as described for Step 1, Example 87, using 2-chloroacetyl chloride and 6-bromo-1-neopentyl-1H-indole. ¹HNMR (CDCl₃, 400 MHz) δ 8.22 (d, J=8.9 Hz, 1H), 7.78 (s, 1H), 7.54 (d, J=1.5 Hz, 1H), 7.40 (dd, J=8.9 Hz, 1.6 Hz, 1H), 4.49 (s, 2H), 3.93 (s, 2H), 1.04 (s, 9H).

### Step 2: 1-(6-bromo-1-neopentyl-1H-indol-3-yl)-2-(cyclopropylsulfonyl)ethan-1-one

Sodium cyclopropanesulfinate (0.48 g, 1.5 equiv.) was added to 1-(6-bromo-1-neopentyl-1H-indol-3-yl)-2-chloroethan-1-one (0.85 g, 1.0 equiv) in DMF (10 mL). The reaction mixture was heated at 90° C. overnight, and the completion of the reaction was monitored by reverse phase analytical HPLC. Water (30 mL) and DCM (30 mL) were added into the flask. The aqueous layer was separated and was extracted with DCM (2×30 mL). The combined organic layers were washed with brine (2×30 mL), dried (Na₂SO₄) and filtered. The solvent was removed under reduced pressure and the residue was purified by combi-flash on silica gel to obtain 0.7 g of the title compound as an oil, yield ˜70%. ESI-MS (m/z): 412.1 [M+1]⁺. HNMR (CDCl₃, 400 MHz) δ 8.24 (d, J=8.9 Hz, 1H), 7.84 (s, 1H), 7.55 (d, J=1.5 Hz, 1H), 7.42 (dd, J₁=8.9 Hz, J₂=1.6 Hz, 1H), 4.47 (s, 2H), 3.94 (s, 2H), 2.70 (m, 2H), 1.24-1.30 (m, 2H), 1.09-1.13 (m, 2H), 1.04 (s, 9H).

### Step 3: 6-bromo-3-(2-(cyclopropylsulfonyl)ethyl)-1-neopentyl-1H-indole

The title compound was prepared following the same general protocol as described for Step 3, Example 1, using 1-(6-bromo-1-neopentyl-1H-indol-3-yl)-2-(cyclopropylsulfonyl)ethan-1-one. ESI-MS (m/z): 400.1 [M+1]⁺.

### Step 4: 3-(2-(cyclopropylsulfonyl)ethyl)-1-neopentyl-6-(4,4,5,5-tetramethyl-1,3,2-dioxaborolan-2-yl)-1H-indole

The title compound was prepared following the same general protocol as described for Step 2, Example 33, using 6-bromo-3-(2-(cyclopropylsulfonyl)ethyl)-1-neopentyl-1H-indole. ESI-MS (m/z): 446.3 [M+1]⁺.

### Step 5: 3-(2-(cyclopropylsulfonyl)ethyl)-6-(4-methyl-2-(trifluoromethyl)phenyl)-1-neopentyl-1H-indole

The title compound was prepared following the same general protocol as described for Step 5, Example 1, using 3-(2-(cyclopropylsulfonyl)ethyl)-1-neopentyl-6-(4,4,5,5-tetramethyl-1,3,2-dioxaborolan-2-yl)-1H-indole and 1-bromo-4-methyl-2-(trifluoromethyl)benzene. ESI-MS (m/z): 478.2 [M+1]⁺.

### Example 409: (S)—N-(1-(6-(2-cyano-6-fluorophenyl)-1-neopentyl-1H-indol-3-yl)-2,2,2-trifluoroethyl)cyclobutanesulfonamide (409)

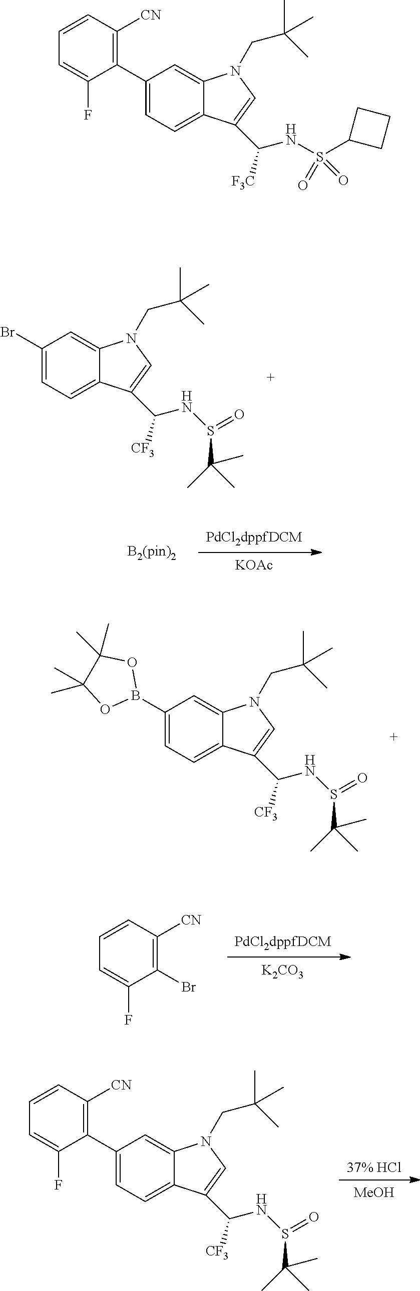

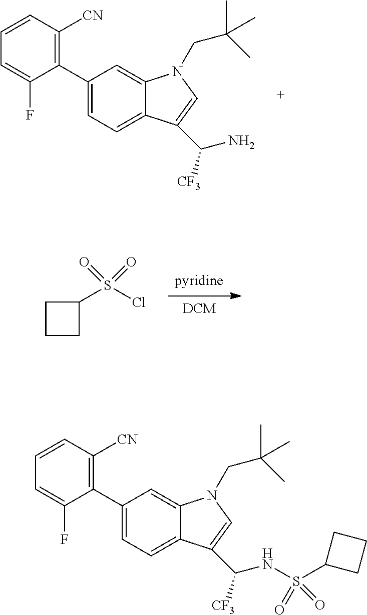

### Step 1: (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide

The title compound was prepared following the same general protocol as described for Step 2, Example 33, using (S)—N—((S)-1-(6-bromo-1-neopentyl-1H-indol-3-yl)-2,2,2-trifluoroethyl)-2-methylpropane-2-sulfinamide. ESI-MS (m/z): 515.3 [M+1]⁺.

### Step 2: (S)—N—((S)-1-(6-(2-cyano-6-fluorophenyl)-1-neopentyl-1H-indol-3-yl)-2,2,2-trifluoroethyl)-2-methylpropane-2-sulfinamide

The title compound was prepared following the same general protocol as described for Step 5, Example 1, using (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide and 2-bromo-3-fluorobenzonitrile. ESI-MS (m/z): 508.2 [M+1]⁺.

### Step 3: (S)-2-(3-(1-amino-2,2,2-trifluoroethyl)-1-neopentyl-1H-indol-6-yl)-3-fluorobenzonitrile

The title compound was prepared following the same general protocol as described for Step 4, Example 116, using (S)—N—((S)-1-(6-(2-cyano-6-fluorophenyl)-1-neopentyl-1H-indol-3-yl)-2,2,2-trifluoroethyl)-2-methylpropane-2-sulfinamide. ESI-MS (m/z): 387.2 [M-NH₂]⁺.

### Step 4: (S)—N-(1-(6-(2-cyano-6-fluorophenyl)-1-neopentyl-1H-indol-3-yl)-2,2,2-trifluoroethyl)cyclobutanesulfonamide

The title compound was prepared following the same general protocol as described for Step 5, Example 116, using (S)-2-(3-(1-amino-2,2,2-trifluoroethyl)-1-neopentyl-1H-indol-6-yl)-3-fluorobenzonitrile and cyclobutanesulfonyl chloride. ESI-MS (m/z): 522.2 [M+1]⁺.

### Example 410: (S)—N-(2,2,2-trifluoro-1-(1-isobutyl-6-(2-(trifluoromethyl)phenyl)-1H-indol-3-yl)ethyl)cyclopropanesulfonamide (410)

### Step 1: 6-bromo-1-isobutyl-1H-indole

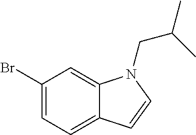

To a stirred solution of 6-bromo-1H-indole (25 g, 127.52 mmol) in DMF (150 mL) was added sodium hydride (20.40 g, 510.09 mmol, 60% purity) in ice-bath under N2. The mixture was stirred at 50° C. for 15 min. Then 1-iodo-2-methyl-propane (46.93 g, 255.05 mmol, 29.35 mL) was added to the mixture. The mixture was stirred at 80° C. for 16 h. The reaction was poured to ice water (150 mL) and extracted with EA (400 mL*2). The combined organic layers were dried over anhydrous Na2SO4, filtered and concentrated. The residue obtained was purified by CC (PE) to give 6-bromo-1-isobutyl-indole (15.6 g, 61.87 mmol, 48.52% yield) as a light-yellow oil.

### Step 2: (S)—N—((S)-1-(6-bromo-1-isobutyl-1H-indol-3-yl)-2,2,2-trifluoroethyl)-2-methylpropane-2-sulfinamide

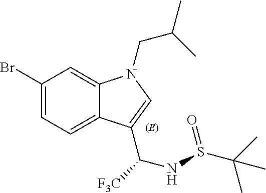 [SMILES: CC(C)Cn1cc([C@H](N[S@@](=O)C(C)(C)C)C(F)(F)F)c2ccc(Br)cc21]

To a stirred solution of 6-bromo-1-isobutyl-indole (5 g, 19.83 mmol) in anhydrous CH₂Cl₂ (17 mL) was added BF3·OEt2 (2.81 g, 19.83 mmol, 2.45 mL) slowly at −10° C. under Nitrogen, followed by addition of a solution of (S,E)-2-methyl-N-(2,2,2-trifluoroethylidene)propane-2-sulfinamide in DCM. The resulting mixture was raised to room temperature for 5 h. Then the mixture was added water. The mixture was separated organic phase, the aqueous phase was extracted with CH₂Cl₂, the combined organic phases were washed with brine, dried with anhydrous Na₂SO₄ and filtered. The filtrate was concentrated to give the crude product, which was purified by prep-HPLC to get (S)—N—((S)-1-(6-bromo-1-isobutyl-1H-indol-3-yl)-2,2,2-trifluoroethyl)-2-methylpropane-2-sulfinamide (3.5 g, 7.72 mmol, 46.72% yield) as a white solid.

### Step 3: (S)-1-(6-bromo-1-isobutyl-1H-indol-3-yl)-2,2,2-trifluoroethanamine

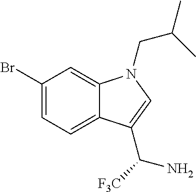 [SMILES: CC(C)Cn1cc([C@H](N)C(F)(F)F)c2ccc(Br)cc21]

A mixture of (S)—N—((S)-1-(6-bromo-1-isobutyl-1H-indol-3-yl)-2,2,2-trifluoroethyl)-2-methylpropane-2-sulfinamide (3.0 g, 6.62 mmol) in 4M HCl(g)/ethanol (30 mL) was stirred at room temperature for 2 hours, the mixture was concentrated, the residue was basified to pH=8 with sat. NaHCO₃, the aqueous phase was extracted with EtOAc twice, the combined organic phases were washed with brine, dried with anhydrous Na₂SO₄ and filtered. The filtrate was concentrated to give the crude product, which was purified by silica gel column (EA/PE=0-50%) to get (S)-1-(6-bromo-1-isobutyl-1H-indol-3-yl)-2,2,2-trifluoroethanamine (2.3 g, 6.59 mmol, 99.54% yield) as yellow oil.

### Step 4: (S)-2,2,2-trifluoro-1-(1-isobutyl-6-(2-(trifluoromethyl)phenyl)-1H-indol-3-yl)ethanamine

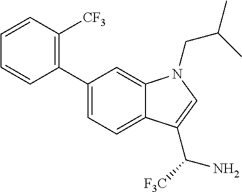 [SMILES: CC(C)Cn1cc([C@H](N)C(F)(F)F)c2ccc(-c3ccccc3C(F)(F)F)cc21]

A mixture of (S)-1-(6-bromo-1-isobutyl-1H-indol-3-yl)-2,2,2-trifluoroethanamine (2.3 g, 6.59 mmol), [2-(trifluoromethyl)phenyl]boronic acid (1.50 g, 7.90 mmol), Pd(dppf)Cl2 (481.95 mg, 658.67 μmol) and Na₂CO₃ (1.75 g, 16.47 mmol) in dioxane (40 mL) and water (10 mL) was stirred at 80° C. overnight under Nitrogen. The reaction mixture was concentrated to remove solvent, to the residue was added water and extracted with ethyl acetate thrice. The combined organic phases were washed with brine, dried with anhydrous Na₂SO₄ and filtered. The filtrate was concentrated to give the crude product, which was purified by silica gel column (EA/PE=0-50%) to get (S)-2,2,2-trifluoro-1-(1-isobutyl-6-(2-(trifluoromethyl)phenyl)-1H-indol-3-yl)ethanamine (2.4 g, 5.79 mmol, 87.93% yield) as yellow oil.

### Step 5: (S)—N-(2,2,2-trifluoro-1-(1-isobutyl-6-(2-(trifluoromethyl)phenyl)-1H-indol-3-yl)ethyl)cyclopropanesulfonamide

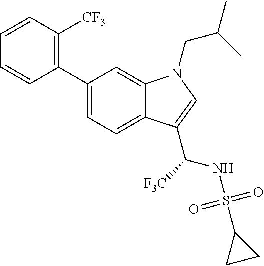 [SMILES: CC(C)Cn1cc([C@H](NS(=O)(=O)C2CC2)C(F)(F)F)c2ccc(-c3ccccc3C(F)(F)F)cc21]

A mixture of(S)-2,2,2-trifluoro-1-(1-isobutyl-6-(2-(trifluoromethyl)phenyl)-1H-indol-3-yl)ethanamine (2.4 g, 5.79 mmol) and cyclopropanesulfonyl chloride (2.44 g, 17.38 mmol, 1.77 mL) in anhydrous pyridine (20 mL) was stirred at room temperature overnight. The mixture was concentrated for remove pyridine to get black oil, which was added 1N HCl and EtOAc. The organic phase was separated, the aqueous phase was extracted with EtOAc twice. The combined organic phases were washed with brine, dried with anhydrous Na2SO4 and filtered. The filtrate was concentrated to get yellow oil, which was purified by silica gel column (EA/PE=0-80%) to get crude product as light yellow oil. Which was purified by prep-HPLC to get (S)—N-(2,2,2-trifluoro-1-(1-isobutyl-6-(2-(trifluoromethyl)phenyl)-1H-indol-3-yl)ethyl)cyclopropanesulfonamide (1.88 g, 3.62 mmol, 62.44% yield) as a white solid. ¹H NMR (400 MHz, DMSO) δ 8.58 (s, 1H), 7.83 (dd, J=7.7, 4.1 Hz, 2H), 7.72 (t, J=7.3 Hz, 1H), 7.68 (s, 1H), 7.60 (t, J=7.7 Hz, 1H), 7.47 (d, J=6.7 Hz, 2H), 7.03 (d, J=8.3 Hz, 1H), 5.53-5.39 (m, 1H), 4.01 (d, J=7.2 Hz, 2H), 2.33 (tt, J=8.0, 4.8 Hz, 1H), 2.11-2.03 (m, 1H), 0.90 (dt, J=9.7, 4.8 Hz, 2H), 0.84 (dd, J=6.5, 5.5 Hz, 6H), 0.78-0.64 (m, 2H). LCMS: 518.7 m/z [M+H]⁺.

### Example 411: (S)—N-(1-(6-(2-chloro-6-fluorophenyl)-1-neopentyl-1H-indol-3-yl)-2,2,2-trifluoroethyl)cyclobutanesulfonamide (411)

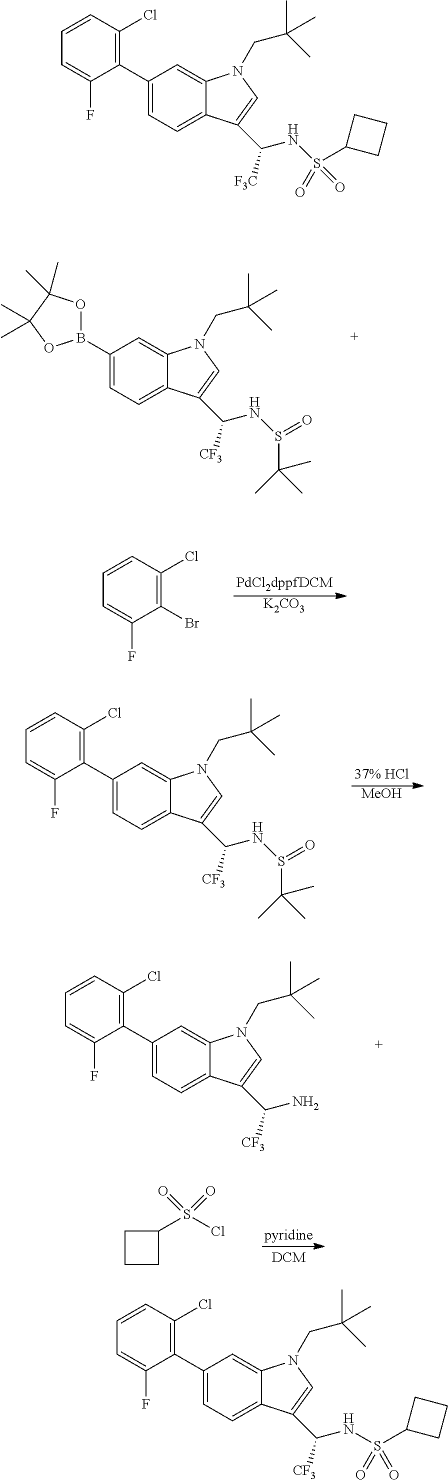

### Step 1: (S)—N—((S)-1-(6-(2-chloro-6-fluorophenyl)-1-neopentyl-1H-indol-3-yl)-2,2,2-trifluoroethyl)-2-methylpropane-2-sulfinamide

The title compound was prepared following the same general protocol as described for Step 5, Example 1, using (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide and 2-bromo-1-chloro-3-fluorobenzene. ESI-MS (m/z): 517.2 [M+1]⁺.

### Step 2: (S)-1-(6-(2-chloro-6-fluorophenyl)-1-neopentyl-1H-indol-3-yl)-2,2,2-trifluoroethan-1-amine

The title compound was prepared following the same general protocol as described for Step 4, Example 116, using (S)—N—((S)-1-(6-(2-chloro-6-fluorophenyl)-1-neopentyl-1H-indol-3-yl)-2,2,2-trifluoroethyl)-2-methylpropane-2-sulfinamide. ESI-MS (m/z): 396.1 [M-NH₂]⁺.

### Step 3: (S)—N-(1-(6-(2-chloro-6-fluorophenyl)-1-neopentyl-1H-indol-3-yl)-2,2,2-trifluoroethyl)cyclobutanesulfonamide

The title compound was prepared following the same general protocol as described for Step 5, Example 116, using (S)-1-(6-(2-chloro-6-fluorophenyl)-1-neopentyl-1H-indol-3-yl)-2,2,2-trifluoroethan-1-amine and cyclobutanesulfonyl chloride. ESI-MS (m/z): 531.2 [M+1]⁺.

### Example 412: The synthesis of N-[rac-(1S)-2,2,2-trifluoro-1-[1-(2-hydroxy-2-methyl-propyl)-6-[2-(trifluoromethyl)phenyl]pyrrolo[2,3-b]pyridin-3-yl]ethyl]cyclobutanesulfonamide (412)

### Step: 1-(6-bromopyrrolo[2,3-b]pyridin-1-yl)-2-methyl-propan-2-ol

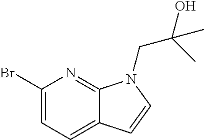

6-bromo-1H-pyrrolo[2,3-b]pyridine (2.5 g, 12.69 mmol) was added to a suspension of sodium hydride (1.01 g, 25.38 mmol, 60% purity) in anhydrous DMF (30 mL) in ice-water under nitrogen. The mixture was stirred for 1 h at room temperature, then cooling in ice-water for the addition of 2,2-dimethyloxirane (2.74 g, 38.06 mmol) and the resulting mixture was stirred at room temperature overnight under nitrogen. After quenching with water the reaction mixture was partitioned between ethyl acetate and water. The aqueous layer was extracted using ethyl acetate and the combined organic layers were washed brine, dried over anhydrous sodium sulfate. After the solvent was removed in vacuo, flash chromatography (EA/PE=0-50%) yielded 1-(6-bromopyrrolo[2,3-b]pyridin-1-yl)-2-methyl-propan-2-ol (3.4 g, 12.63 mmol, 99.56% yield) as yellow oil.

### Step 2: (S)—N—((S)-1-(6-bromo-1-(2-hydroxy-2-methylpropyl)-1H-pyrrolo[2,3-b]pyridin-3-yl)-2,2,2-trifluoroethyl)-2-methylpropane-2-sulfinamide

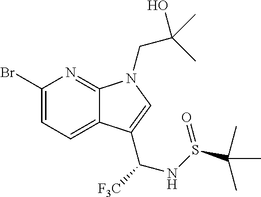 [SMILES: CC(C)(O)Cn1cc([C@H](N[S@@](=O)C(C)(C)C)C(F)(F)F)c2ccc(Br)nc21]

The mixture of 1-(6-bromopyrrolo[2,3-b]pyridin-1-yl)-2-methyl-propan-2-ol (900 mg, 3.34 mmol) in anhydrous CH₂Cl₂ (6 mL) was cooled to −10° C., BF₃·OEt₂ (2.85 g, 20.06 mmol, 2.48 mL) was added slowly, followed by addition of a solution of (S,E)-2-methyl-N-(2,2,2-trifluoroethylidene)propane-2-sulfinamide in DCM. The resulting mixture was heated to reflux overnight under Nitrogen. The mixture was diluted with DCM, followed by water. The aqueous layer was extracted with DCM twice. Organic layers were combined and washed with brine and concentrated in vacuo to give the crude product as brown oil, directly next step.

### Step 3: 1-[6-bromo-3-[rac-(1S)-1-amino-2,2,2-trifluoro-ethyl]pyrrolo[2,3-b]pyridin-1-yl]-2-methyl-propan-2-ol

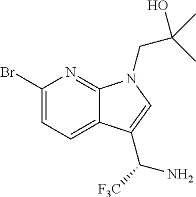 [SMILES: CC(C)(O)Cn1cc([C@H](N)C(F)(F)F)c2ccc(Br)nc21]

A mixture of N-[1-[6-bromo-1-(2-hydroxy-2-methyl-propyl)pyrrolo[2,3-b]pyridin-3-yl]-2,2,2-trifluoro-ethyl]-2-methyl-propane-2-sulfinamide (1.57 g, 3.34 mmol) in 4M HCl(g)/ethanol (20 mL) was stirred at room temperature for 2 hours, the mixture was concentrated, the residue was basified to pH=8 with sat. NaHCO₃, the aqueous phase was extracted with EtOAc twice, the combined organic phases were washed with brine, dried with anhydrous Na₂SO₄ and filtered. The filtrate was concentrated to give the crude product, which was purified by silica gel column (EA/PE=0-80%) to get 1-[6-bromo-3-[rac-(1S)-1-amino-2,2,2-trifluoro-ethyl]pyrrolo[2,3-b]pyridin-1-yl]-2-methyl-propan-2-ol (870 mg, 2.38 mmol, 71.13% yield) as yellow oil.

### Step 4: The synthesis of 2-methyl-1-[3-[rac-(1S)-1-amino-2,2,2-trifluoro-ethyl]-6-[2-(trifluoromethyl)phenyl]pyrrolo[2,3-b]pyridin-1-yl]propan-2-ol

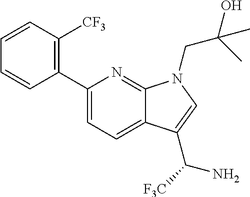 [SMILES: CC(C)(O)Cn1cc([C@H](N)C(F)(F)F)c2ccc(-c3ccccc3C(F)(F)F)nc21]

A mixture of 1-[3-(1-amino-2,2,2-trifluoro-ethyl)-6-bromo-pyrrolo[2,3-b]pyridin-1-yl]-2-methyl-propan-2-ol (870 mg, 2.38 mmol), [2-(trifluoromethyl)phenyl]boronic acid (586.62 mg, 3.09 mmol), Pd(PPh₃)₄ (164.73 mg, 142.55 μmol) and K₂CO₃ (656.73 mg, 4.75 mmol) in dioxane (10 mL) and Water (2.5 mL) was stirred at 80° C. overnight under Nitrogen. The reaction mixture was added water and extracted with ethyl acetate thrice. The combined organic phases were washed with brine, dried with anhydrous Na₂SO₄ and filtered. The filtrate was concentrated to give the crude product, which was purified by silica gel column (EA/PE=0-80%) to get 2-methyl-1-[3-[rac-(1S)-1-amino-2,2,2-trifluoro-ethyl]-6-[2-(trifluoromethyl)phenyl]pyrrolo[2,3-b]pyridin-1-yl]propan-2-ol (775 mg, 1.80 mmol, 75.62% yield) as yellow oil.

### Step 5: N-[rac-(1S)-2,2,2-trifluoro-1-[1-(2-hydroxy-2-methyl-propyl)-6-[2-(trifluoromethyl)phenyl]pyrrolo[2,3-b]pyridin-3-yl]ethyl]cyclobutanesulfonamide

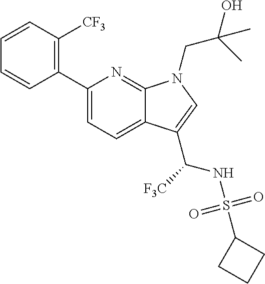 [SMILES: CC(C)(O)Cn1cc([C@H](NS(=O)(=O)C2CCC2)C(F)(F)F)c2ccc(-c3ccccc3C(F)(F)F)nc21]

A mixture of 2-methyl-1-[3-[rac-(1S)-1-amino-2,2,2-trifluoro-ethyl]-6-[2-(trifluoromethyl)phenyl]pyrrolo[2,3-b]pyridin-1-yl]propan-2-ol (383.46 mg, 565 μmol) and cyclobutanesulfonyl chloride (349.43 mg, 2.26 mmol) in anhydrous Pyridine (0.8 mL) was stirred at 100° C. for 4 hours in the microwave. The reaction liquid was checked by LC-MS, the mixture was concentrated for remove pyridine to get black oil, which was purified by silica gel column (EA/PE=0-80%) to get crude product as brown oil, which was purified by prep-HPLC to get N-[rac-(1S)-2,2,2-trifluoro-1-[1-(2-hydroxy-2-methyl-propyl)-6-[2-(trifluoromethyl)phenyl]pyrrolo[2,3-b]pyridin-3-yl]ethyl]cyclobutanesulfonamide (80.33 mg, 146.18 μmol, 25.87% yield) as a off-white solid. ¹H NMR (400 MHz, DMSO) δ 8.70 (s, 1H), 8.34 (d, J=8.1 Hz, 1H), 7.94 (s, 1H), 7.86 (d, J=7.8 Hz, 1H), 7.76 (t, J=7.4 Hz, 1H), 7.66 (t, J=7.6 Hz, 1H), 7.59 (d, J=7.6 Hz, 1H), 7.27 (d, J=8.2 Hz, 1H), 5.53 (d, J=7.0 Hz, 1H), 4.82 (s, 1H), 4.21 (s, 2H), 3.76-3.62 (m, 1H), 2.30-2.13 (m, 2H), 2.01-1.85 (m, 2H), 1.80-1.65 (m, 2H), 1.04 (s, 6H). LCMS: 549.8 m/z [M+H]⁺.

### Example 413: (S)—N-(2,2,2-trifluoro-1-(1-(2-methoxy-2-methylpropyl)-6-(2-(trifluoromethyl)phenyl)-1H-pyrrolo[2,3-b]pyridin-3-yl)ethyl)cyclobutanesulfonamide (413)

### Step 1: 6-bromo-1-(2-methoxy-2-methyl-propyl)pyrrolo[2,3-b]pyridine

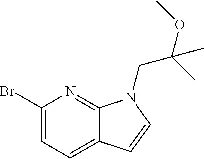 [SMILES: COC(C)(C)Cn1ccc2ccc(Br)nc21]

1-(6-bromopyrrolo[2,3-b]pyridin-1-yl)-2-methyl-propan-2-ol (1.0 g, 3.72 mmol) was added to a suspension of sodium hydride (520.13 mg, 13.00 mmol, 60% purity) in anhydrous DMF (20 mL) in ice-water under nitrogen. The mixture was stirred for 1 h at room temperature, then cooling in ice-water for the addition of iodomethane (3.69 g, 26.01 mmol, 1.62 mL) and the resulting mixture was stirred at room temperature overnight under nitrogen. To the reaction mixture was added water and extracted with ethyl acetate. The combined organic phases were washed with brine, dried with anhydrous Na2SO4 and filtered. The filtrate was concentrated to give the crude product, which was purified by silica gel column (EA/PE=0-25%) to get 6-bromo-1-(2-methoxy-2-methyl-propyl)pyrrolo[2,3-b]pyridine (937 mg, 3.31 mmol, 89.06% yield) as light yellow oil.

### Step 2: N—((S)-1-(6-bromo-1-(2-methoxy-2-methylpropyl)-1H-pyrrolo[2,3-b]pyridin-3-yl)-2,2,2-trifluoroethyl)-2-methylpropane-2-sulfinamide

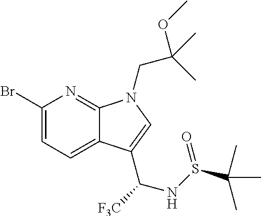 [SMILES: COC(C)(C)Cn1cc([C@H](N[S@@](=O)C(C)(C)C)C(F)(F)F)c2ccc(Br)nc21]

The mixture of 6-bromo-1-(2-methoxy-2-methyl-propyl)pyrrolo[2,3-b]pyridine (937 mg, 3.31 mmol) in anhydrous CH₂Cl₂ (6 mL) was cooled to −10° C., BF₃·OEt₂ (2.82 g, 19.86 mmol) was added slowly, followed by addition of a solution of (S,E)-2-methyl-N-(2,2,2-trifluoroethylidene)propane-2-sulfinamide in DCM. The resulting mixture was heated to reflux overnight under Nitrogen. The mixture was diluted with DCM, followed by water. The aqueous layer was extracted with DCM twice. Organic layers were combined and washed with brine and concentrated in vacuo to give the crude product as brown oil, directly next step.

### Step 3: 1-[6-bromo-1-(2-methoxy-2-methyl-propyl)pyrrolo[2,3-b]pyridin-3-yl]-2,2,2-trifluoro-ethanamine

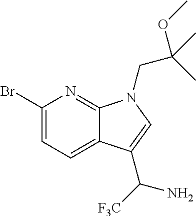 [SMILES: COC(C)(C)Cn1cc(C(N)C(F)(F)F)c2ccc(Br)nc21]

A mixture of 2-methyl-N-[1-[6-bromo-1-(2-methoxy-2-methyl-propyl)pyrrolo[2,3-b]pyridin-3-yl]-2,2,2-trifluoro-ethyl]propane-2-sulfinamide (1.60 g, 3.31 mmol) in 4M HCl(g)/ethanol (20 mL) was stirred at room temperature for 2 hours, the mixture was concentrated, the residue was basified to pH=8 with sat. NaHCO3, the aqueous phase was extracted with EtOAc twice, the combined organic phases were washed with brine, dried with anhydrous Na2SO4 and filtered. The filtrate was concentrated to give the crude product, which was purified by silica gel column (EA/PE=0-80%) to get crude, which was purified by prep-HPLC to get 1-[6-bromo-1-(2-methoxy-2-methyl-propyl)pyrrolo[2,3-b]pyridin-3-yl]-2,2,2-trifluoro-ethanamine (366 mg, 962.64 μmol, 29.08% yield) as yellow oil.

### Step 4: 2,2,2-trifluoro-1-[1-(2-methoxy-2-methyl-propyl)-6-[2-(trifluoromethyl)phenyl]pyrrolo[2,3-b]pyridin-3-yl]ethanamine

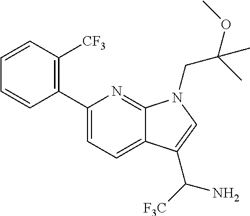 [SMILES: COC(C)(C)Cn1cc(C(N)C(F)(F)F)c2ccc(-c3ccccc3C(F)(F)F)nc21]

A mixture of 1-[6-bromo-1-(2-methoxy-2-methyl-propyl)pyrrolo[2,3-b]pyridin-3-yl]-2,2,2-trifluoro-ethanamine (204 mg, 536.56 μmol), [2-(trifluoromethyl)phenyl]boronic acid (132.48 mg, 697.52 μmol), Pd(PPh₃)₄ (49.60 mg, 42.92 μmol) in dioxane (4 mL) and water (1 mL) was stirred at 80° C. for 6 hours under Nitrogen. The reaction mixture was added water and extracted with ethyl acetate thrice. The combined organic phases were washed with brine, dried with anhydrous Na₂SO₄ and filtered. The filtrate was concentrated to give the crude product, which was purified by silica gel column (EA/PE=0-80%) to give 2,2,2-trifluoro-1-[1-(2-methoxy-2-methyl-propyl)-6-[2-(trifluoromethyl)phenyl]pyrrolo[2,3-b]pyridin-3-yl]ethanamine (202 mg, 453.52 μmol, 84.53% yield) as yellow oil.

### Step 5: (2,2,2-trifluoro-1-(1-(2-methoxy-2-methylpropyl)-6-(2-(trifluoromethyl)phenyl)-1H-pyrrolo[2,3-b]pyridin-3-yl)ethyl)cyclobutanesulfonamide

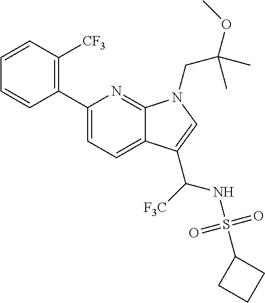 [SMILES: COC(C)(C)Cn1cc(C(NS(=O)(=O)C2CCC2)C(F)(F)F)c2ccc(-c3ccccc3C(F)(F)F)nc21]

A mixture of 2,2,2-trifluoro-1-(1-(2-methoxy-2-methylpropyl)-6-(2-(trifluoromethyl)phenyl)-1H-pyrrolo[2,3-b]pyridin-3-yl)ethanamine (202 mg, 453.52 μmol) and cyclobutanesulfonyl chloride (280.49 mg, 1.81 mmol) in anhydrous pyridine (0.6 mL) was stirred at 100° C. for 4 hours in the microwave. The reaction liquid was checked by LC-MS, the mixture was concentrated for remove pyridine to get black oil, which was purified by silica gel column (EA/PE=0-80%) to get crude product as brown oil, which was purified by prep-HPLC to get (S)—N-(2,2,2-trifluoro-1-(1-(2-methoxy-2-methylpropyl)-6-(2-(trifluoromethyl)phenyl)-1H-pyrrolo[2,3-b]pyridin-3-yl)ethyl)cyclobutanesulfonamide (33.27 mg, 59.04 μmol, 13.02% yield) as a white solid. ¹H NMR (400 MHz, DMSO) δ 8.74 (d, J=9.4 Hz, 1H), 8.36 (d, J=8.1 Hz, 1H), 7.89 (t, J=3.6 Hz, 2H), 7.78 (t, J=7.4 Hz, 1H), 7.68 (t, J=7.6 Hz, 1H), 7.62 (d, J=7.6 Hz, 1H), 7.30 (d, J=8.2 Hz, 1H), 5.63-5.48 (m, 1H), 4.40-4.22 (m, 2H), 3.69 (p, J=8.1 Hz, 1H), 3.23 (s, 3H), 2.31-2.11 (m, 2H), 2.06-1.86 (m, 2H), 1.82-1.65 (m, 2H), 1.05 (d, J=6.5 Hz, 6H). LCMS: 563.7 m/z [M+H]⁺.

### Example 414: (S)—N-(1-(1-((1-cyanocyclopropyl)methyl)-6-(2-(trifluoromethyl)phenyl)-1H-pyrrolo[2,3-b]pyridin-3-yl)-2,2,2-trifluoroethyl)cyclobutanesulfonamide (414)

### Step 1: (1-cyanocyclopropyl)methyl methanesulfonate

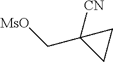 [SMILES: CS(=O)(=O)OCC1(C#N)CC1]

To a stirred solution of 1-(hydroxymethyl)cyclopropanecarbonitrile (1 g, 10.30 mmol) and Triethylamine (3.13 g, 30.89 mmol, 4.31 mL) in anhydrous CH₂Cl₂ (25 mL) was added methanesulfonyl chloride (1.77 g, 15.45 mmol, 1.20 mL) in portions under ice-bath. The mixture was stirred at room temperature overnight under nitrogen, diluted with CH₂C12 and washed with 1 M aqueous HCl solution and brine. After drying (Na₂SO₄) the solvent is evaporated give (1-cyanocyclopropyl)methyl methanesulfonate (1.50 g, 8.56 mmol) as a yellow liquid.

### Step 2: 1-[(6-bromopyrrolo[2,3-b]pyridin-1-yl)methyl]cyclopropanecarbonitrile

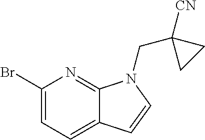 [SMILES: N#CC1(Cn2ccc3ccc(Br)nc32)CC1]

6-bromo-1H-pyrrolo[2,3-b]pyridine (800 mg, 4.06 mmol) and potassium iodide (1.35 g, 8.12 mmol, 432.03 uL) in DMF (30 mL) was added NaH (487.15 mg, 12.18 mmol, 60% purity) under ice-bath. The mixture was stirred for 1 hour and heated to 50° C. under N2. Then (1-cyanocyclopropyl)methyl methanesulfonate (1.42 g, 8.12 mmol) in DMF (5 mL) was injected to reaction. The reaction was stirred at 80° C. overnight under N2. The mixture was cooled to RT and quenched with water and extracted with EtOAc three times. The combined organic layers were washed with brine twice and dried over anhydrous Na₂SO₄, filtered and concentrated. The residue obtained was purified by flash (PE/EA from 0% to 20%) to give 1-[(6-bromopyrrolo[2,3-b]pyridin-1-yl)methyl]cyclopropanecarbonitrile (1.25 g, 4.53 mmol) as a light-yellow solid.

### Step 3: rac-(S)—N—((S)-1-(6-bromo-1-((1-cyanocyclopropyl)methyl)-1H-pyrrolo[2,3-b]pyridin-3-yl)-2,2,2-trifluoroethyl)-2-methylpropane-2-sulfinamide

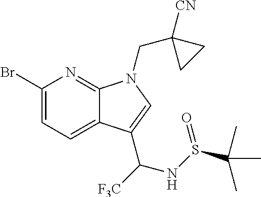 [SMILES: CC(C)(C)[S@](=O)NC(c1cn(CC2(C#N)CC2)c2nc(Br)ccc12)C(F)(F)F]

The mixture of 1-[(6-bromopyrrolo[2,3-b]pyridin-1-yl)methyl]cyclopropanecarbonitrile (650 mg, 2.35 mmol) in anhydrous CH₂Cl₂ (12 mL) was cooled to −10° C., BF₃·OEt₂ (1.00 g, 7.06 mmol, 871.54 μL) was added slowly, followed by addition of a solution of (S,E)-2-methyl-N-(2,2,2-trifluoroethylidene)propane-2-sulfinamide in DCM. The resulting mixture was heated to reflux for 6 h. The mixture was diluted with DCM, followed by water. The aqueous layer was extracted with DCM twice. Organic layers were combined and washed with brine and concentrated in vacuo to give the crude product as brown oil, directly next step.

### Step 4: 1-[[6-bromo-3-[rac-(1S)-1-amino-2,2,2-trifluoro-ethyl]pyrrolo[2,3-b]pyridin-1-yl]methyl]cyclopropanecarbonitrile

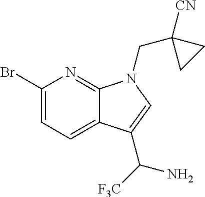 [SMILES: N#CC1(Cn2cc(C(N)C(F)(F)F)c3ccc(Br)nc32)CC1]

A mixture of rac-(S)-2-methyl-N-[rac-(1S)-1-[6-bromo-1-[(1-cyanocyclopropyl)methyl]pyrrolo[2,3-b]pyridin-3-yl]-2,2,2-trifluoro-ethyl]propane-2-sulfinamide (1.12 g, 2.35 mmol) in 4M HCl/EtOH (25 mL) was stirred at RT for 2 hours, the mixture was concentrated, the residue was basified to pH=8 with sat. NaHCO₃, the aqueous phase was extracted with EtOAc twice, the combined organic phases were washed with brine, dried with anhydrous Na₂SO₄ and filtered. The filtrate was concentrated to give the crude product, which was purified by silica gel column (EA/PE=0-50%) to get 1-[[6-bromo-3-[rac-(1S)-1-amino-2,2,2-trifluoro-ethyl]pyrrolo[2,3-b]pyridin-1-yl]methyl]cyclopropanecarbonitrile (810 mg, 2.17 mmol, 92.37% yield) as yellow oil.

### Step 5: 1-[[3-[rac-(1S)-1-amino-2,2,2-trifluoro-ethyl]-6-[2-(trifluoromethyl)phenyl]pyrrolo[2,3-b]pyridin-1-yl]methyl]cyclopropanecarbonitrile

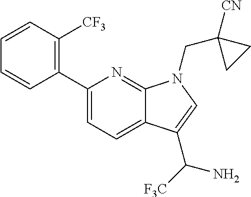 [SMILES: N#CC1(Cn2cc(C(N)C(F)(F)F)c3ccc(-c4ccccc4C(F)(F)F)nc32)CC1]

A mixture of 1-[[6-bromo-3-[rac-(1S)-1-amino-2,2,2-trifluoro-ethyl]pyrrolo[2,3-b]pyridin-1-yl]methyl]cyclopropanecarbonitrile (810 mg, 2.17 mmol), [2-(trifluoromethyl)phenyl]boronic acid (535.93 mg, 2.82 mmol) and K2CO3 (599.97 mg, 4.34 mmol) in dioxane (10 mL) and water (2.5 mL) was stirred at 80° C. overnight under Nitrogen. The mixture was added water and EtOAc. The mixture was stirred and separated. The aqueous phase was extracted with EtOAc twice. The combined organic phases were washed with brine, dried with anhydrous Na₂SO₄ and filtered. The filtrate was concentrated to get brown oil, which was purified by silica gel column (EA/PE=0-80%) to get 1-[[3-[rac-(1S)-1-amino-2,2,2-trifluoro-ethyl]-6-[2-(trifluoromethyl)phenyl]pyrrolo[2,3-b]pyridin-1-yl]methyl]cyclopropanecarbonitrile (920 mg, 2.10 mmol, 96.69% yield) as brown oil.

### Step 6: rac-(S)—N-(1-(1-((1-cyanocyclopropyl)methyl)-6-(2-(trifluoromethyl)phenyl)-1H-pyrrolo[2,3-b]pyridin-3-yl)-2,2,2-trifluoroethyl)cyclobutanesulfonamide

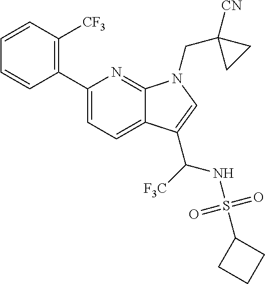 [SMILES: N#CC1(Cn2cc(C(NS(=O)(=O)C3CCC3)C(F)(F)F)c3ccc(-c4ccccc4C(F)(F)F)nc32)CC1]

A mixture of 1-[[3-[rac-(1S)-1-amino-2,2,2-trifluoro-ethyl]-6-[2-(trifluoromethyl)phenyl]pyrrolo[2,3-b]pyridin-1-yl]methyl]cyclopropanecarbonitrile (450 mg, 545.09 μmol) and cyclobutanesulfonyl chloride (400 mg, 2.59 mmol) in anhydrous pyridine (1 mL) was stirred at 100° C. for 4 hours in the microwave. The reaction liquid was checked by LC-MS, the mixture was cooled to room temperature, added ethyl acetate and filtered through silica gel. The filtrate was concentrated to get black oil, which was purified by prep-HPLC to get rac-(S)—N-(1-(1-((1-cyanocyclopropyl)methyl)-6-(2-(trifluoromethyl)phenyl)-1H-pyrrolo[2,3-b]pyridin-3-yl)-2,2,2-trifluoroethyl)cyclobutanesulfonamide (25.77 mg, 46.31 μmol, 8.50% yield) as a light-yellow solid. ¹H NMR (400 MHz, DMSO) δ 8.73 (s, 1H), 8.40 (d, J=8.2 Hz, 1H), 8.06 (s, 1H), 7.88 (d, J=7.5 Hz, 1H), 7.78 (t, J=7.3 Hz, 1H), 7.71-7.62 (m, 2H), 7.33 (d, J=8.2 Hz, 1H), 5.57 (d, J=6.9 Hz, 1H), 4.46 (dd, J=30.7, 14.8 Hz, 2H), 3.75 (d, J=8.1 Hz, 1H), 2.21 (dd, J=19.2, 9.7 Hz, 2H), 2.01-1.86 (m, 2H), 1.81-1.64 (m, 2H), 1.39-1.28 (m, 4H). LCMS: 556.8 m/z [M+H]⁺.

### Example 415: (S)—N-(2,2,2-trifluoro-1-(6-isopropyl-1-neopentyl-1H-indol-3-yl)ethyl)cyclobutanesulfonamide (415)

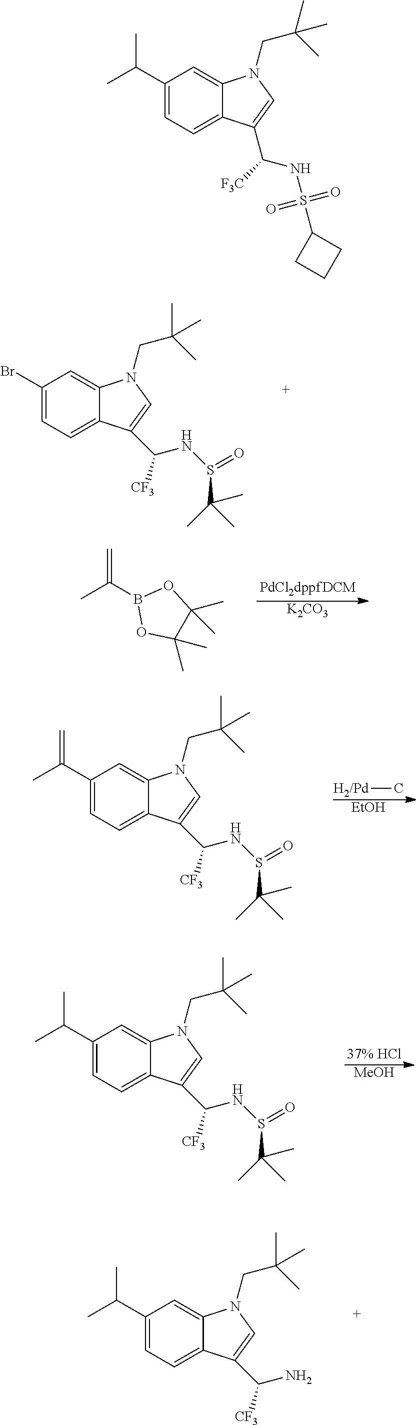

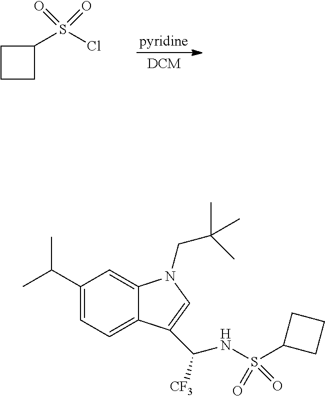 [SMILES: O=S(=O)(Cl)C1CCC1 ; CC(C)c1ccc2c([C@H](NS(=O)(=O)C3CCC3)C(F)(F)F)cn(CC(C)(C)C)c2c1]

### Step 1: (S)-2-methyl-N—((S)-2,2,2-trifluoro-1-(1-neopentyl-6-(prop-1-en-2-yl)-1H-indol-3-yl)ethyl)propane-2-sulfinamide

The title compound was prepared following the same general protocol as described for Step 5, Example 1, using (S)—N—((S)-1-(6-bromo-1-neopentyl-1H-indol-3-yl)-2,2,2-trifluoroethyl)-2-methylpropane-2-sulfinamide and 4,4,5,5-tetramethyl-2-(prop-1-en-2-yl)-1,3,2-dioxaborolane. ESI-MS (m/z): 429.2 [M+1]⁺.

### Step 2: (S)-2-methyl-N—((S)-2,2,2-trifluoro-1-(6-isopropyl-1-neopentyl-1H-indol-3-yl)ethyl)propane-2-sulfinamide

The title compound was prepared following the same general protocol as described for Example 20, using (S)-2-methyl-N—((S)-2,2,2-trifluoro-1-(1-neopentyl-6-(prop-1-en-2-yl)-1H-indol-3-yl)ethyl)propane-2-sulfinamide. ESI-MS (m/z): 431.2 [M+1]⁺.

### Step 3: (S)-2,2,2-trifluoro-1-(6-isopropyl-1-neopentyl-1H-indol-3-yl)ethan-1-amine

The title compound was prepared following the same general protocol as described for Step 4, Example 116, using (S)-2-methyl-N—((S)-2,2,2-trifluoro-1-(6-isopropyl-1-neopentyl-1H-indol-3-yl)ethyl)propane-2-sulfinamide. ESI-MS (m/z): 310.2 [M-NH₂]⁺.

### Step 4: (S)—N-(2,2,2-trifluoro-1-(6-isopropyl-1-neopentyl-1H-indol-3-yl)ethyl)cyclo-butanesulfonamide

The title compound was prepared following the same general protocol as described for Step 5, Example 116, using (S)-1-(6-(2-chloro-6-fluorophenyl)-1-neopentyl-1H-indol-3-yl)-2,2,2-trifluoroethan-1-amine and cyclobutanesulfonyl chloride. ESI-MS (m/z): 445.2 [M+1]⁺.

### Example 416: (S)—N-(2,2,2-trifluoro-1-(1-neopentyl-6-(trifluoromethyl)-1H-indol-3-yl)ethyl)cyclobutanesulfonamide (416)

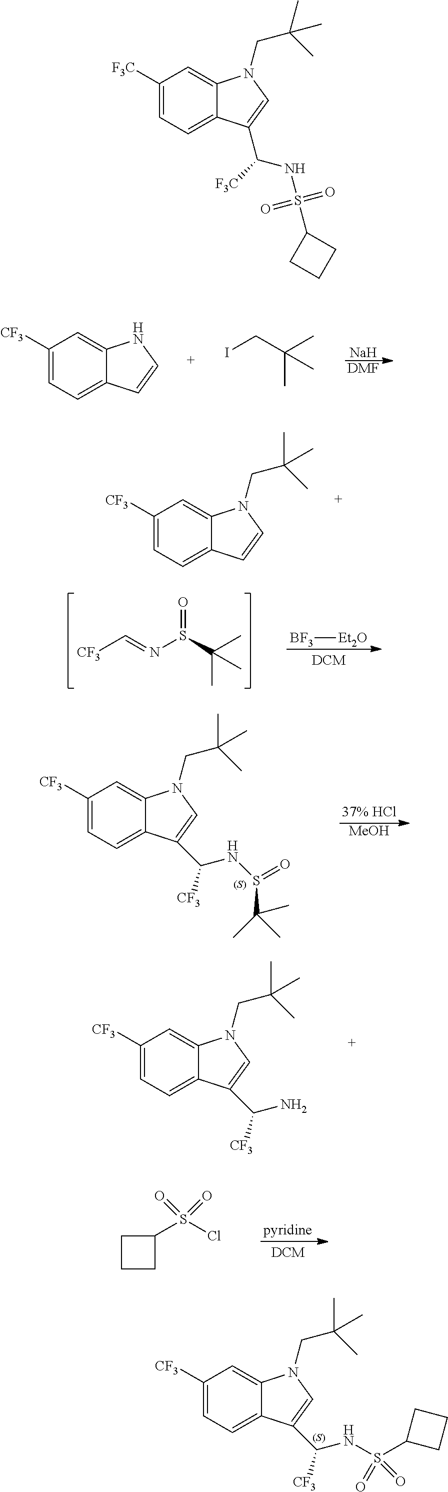

### Step1: 1-neopentyl-6-(trifluoromethyl)-1H-indole

The title compound was prepared following the same general protocol as described for Step 1, Example 1, using 6-(trifluoromethyl)-1H-indole and 1-iodo-2,2-dimethylpropane and 1-iodo-2,2-dimethylpropane. After purification, the product was used in next step directly.

### Step2. (S)-2-methyl-N—((S)-2,2,2-trifluoro-1-(1-neopentyl-6-(trifluoromethyl)-1H-indol-3-yl)ethyl)propane-2-sulfinamide

The title compound was prepared following the same general protocol as described for Step 3, Example 116, using 6-(trifluoromethyl)-1H-indole. ESI-MS (m/z): 457.2 [M+1]⁺.

### Step 3: (S)-2,2,2-trifluoro-1-(1-neopentyl-6-(trifluoromethyl)-1H-indol-3-yl)ethan-1-amine

The title compound was prepared following the same general protocol as described for Step 4, Example 116, using (S)-2-methyl-N—((S)-2,2,2-trifluoro-1-(1-neopentyl-6-(trifluoromethyl)-1H-indol-3-yl)ethyl)propane-2-sulfinamide. ESI-MS (m/z): 336.2 [M-NH₂]⁺.

### Step 4: (S)—N-(2,2,2-trifluoro-1-(1-neopentyl-6-(trifluoromethyl)-1H-indol-3-yl)ethyl)cyclo-butanesulfonamide

The title compound was prepared following the same general protocol as described for Step 5, Example 116, using (S)-2,2,2-trifluoro-1-(1-neopentyl-6-(trifluoromethyl)-1H-indol-3-yl)ethan-1-amine and cyclobutanesulfonyl chloride. ESI-MS (m/z): 471.2 [M+1]⁺.

### Example 417: N-(2-(5-(2,4-bis(trifluoromethyl)phenyl)-3-neopentyl-1H-indol-1-yl)ethyl)propane-2-sulfonamide (417)

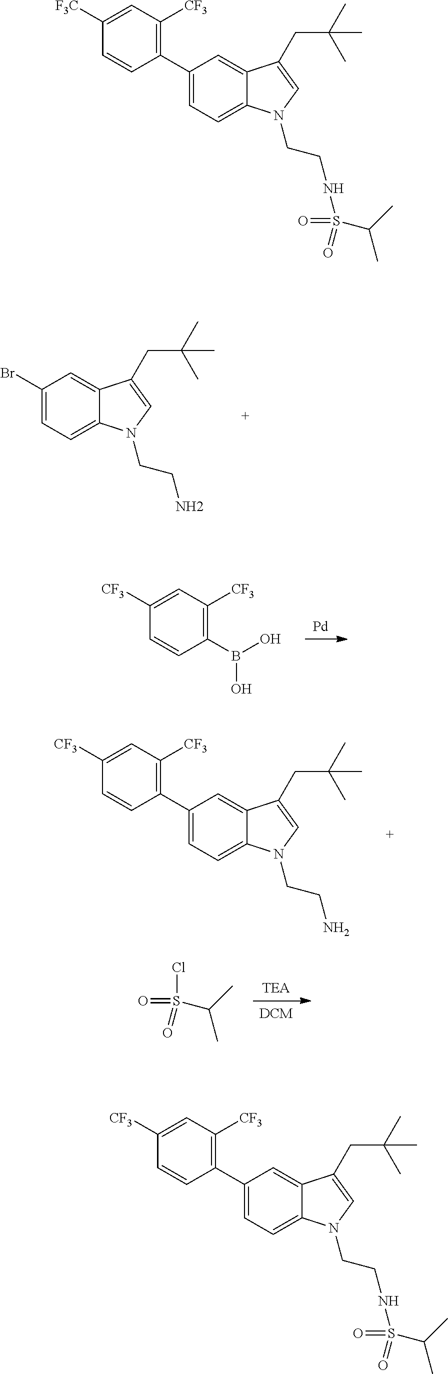

### Step 1: 2-(5-(2,4-bis(trifluoromethyl)phenyl)-3-neopentyl-1H-indol-1-yl)ethan-1-amine

The title compound was prepared following the same general protocol as described for Step 5, Example 1, using 2-(5-bromo-3-neopentyl-1H-indol-1-yl)ethan-1-amine and (2,4-bis-(trifluoromethyl)phenyl)boronic acid. ESI-MS (m/z): 443.2 [M+1]⁺.

### Step 2: 2-(5-(2,4-bis(trifluoromethyl)phenyl)-3-neopentyl-1H-indol-1-yl)ethan-1-amine

The title compound was prepared following the same general protocol as described for Step 4, Example 218, using 2-(5-(2,4-bis(trifluoromethyl)phenyl)-3-neopentyl-1H-indol-1-yl)ethan-1-amine and propane-2-sulfonyl chloride. ESI-MS (m/z): 549.2 [M+1]⁺.

### Example 418: N-(2-(5-(2,4-bis(trifluoromethyl)phenyl)-3-neopentyl-1H-indol-1-yl)ethyl)cyclobutanesulfonamide (418)

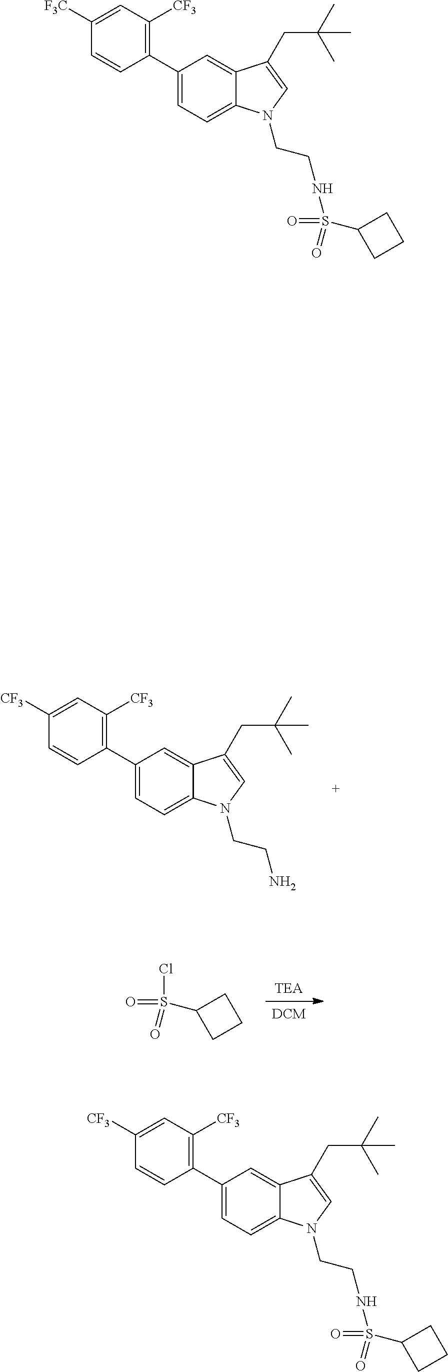

The title compound was prepared following the same general protocol as described for Step 4, Example 218, using 2-(5-(2,4-bis(trifluoromethyl)phenyl)-3-neopentyl-1H-indol-1-yl)ethan-1-amine and cyclobutanesulfonyl chloride. ESI-MS (m/z): 561.3 [M+1]⁺.

### Example 419: (S)-3,3-difluoro-N-(2,2,2-trifluoro-1-(1-neopentyl-6-(2-(trifluoromethyl)phenyl)-1H-indol-3-yl)ethyl)cyclobutane-1-sulfonamide (419)

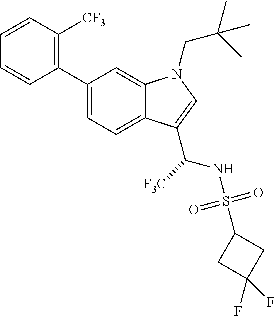 [SMILES: CC(C)(C)Cn1cc([C@H](NS(=O)(=O)C2CC(F)(F)C2)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 3,3-difluorocyclobutane-1-sulfonyl chloride. ¹HNMR (CDCl₃, 400 MHz) δ 7.76 (d, J=8.0 Hz, 1H), 7.65 (d, J=8.0 Hz, 1H), 7.57 (t, J=8.0 Hz, 1H), 7.48 (t, J=8.0 Hz, 1H), 7.39-7.29 (m, 3H), 7.14 (t, J=8.0 Hz, 1H), 5.30-5.21 (m, 1H), 4.58-4.28 (m, 1H), 3.99-3.83 (m, 3H), 3.64-2.92 (m, 4H), 0.99 (s, 9H)

### Example 420: N-(1-(1-neopentyl-6-(2-(trifluoromethyl)phenyl)-1H-indol-3-yl)ethyl)-cyclopropanesulfonamide (420)

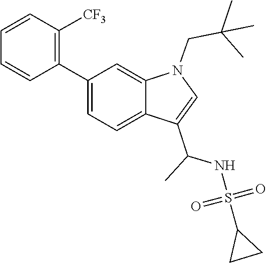 [SMILES: CC(NS(=O)(=O)C1CC1)c1cn(CC(C)(C)C)c2cc(-c3ccccc3C(F)(F)F)ccc12]

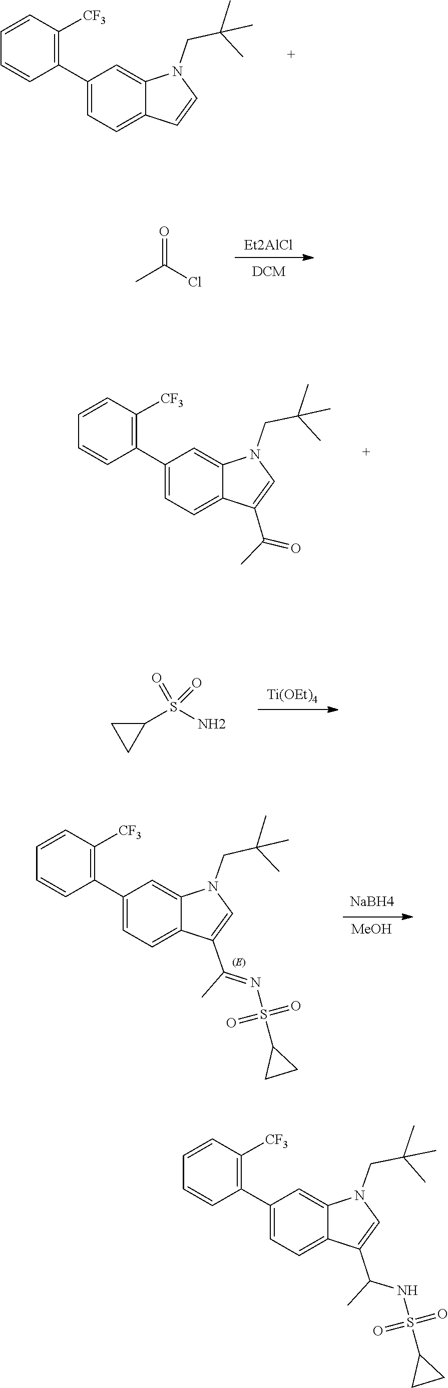

### Step 1: 1-(1-neopentyl-6-(2-(trifluoromethyl)phenyl)-1H-indol-3-yl)ethan-1-one

The title compound was prepared following the same general protocol as described for Step 1, Example 87, using 1-neopentyl-6-(2-(trifluoromethyl)phenyl)-1H-indole and acetyl chloride. ESI-MS (m/z): 389.2 [M+1]⁺.

### Step 2: (E)-N-(1-(1-neopentyl-6-(2-(trifluoromethyl)phenyl)-1H-indol-3-yl)ethylidene)cyclopropanesulfonamide

The title compound was prepared following the same general protocol as described for Step 4, Example 113, using 1-(1-neopentyl-6-(2-(trifluoromethyl)phenyl)-1H-indol-3-yl)ethan-1-one and cyclopropanesulfonamide. ESI-MS (m/z): 477.2 [M+1]⁺.

### Step 3: N-(1-(1-neopentyl-6-(2-(trifluoromethyl)phenyl)-1H-indol-3-yl)ethyl)cyclopropanesulfonamide

NaBH₄ (4 mg, 2 equiv.) was added to a solution of imine (24 mg, 1 equiv.) in MeOH (1 mL) at 0° C. and the reaction mixture was stirred at room temperature for 1-2 hours. The solvent was removed under reduced pressure and the residue was dissolved in EtOAc (5 mL) and water (2 mL). The layers were separated and the aqueous layer was extracted with EtOAc (2×3 mL). The combined organics were dried (Na₂SO₄) and filtered. The solvent was removed under reduced pressure and the residue was purified by combi-flash on silica gel to obtain the title compound as an oil. ESI-MS (m/z): 501.2 [M+Na]⁺.

### Example 421: (S)—N-(1-(1-(cyclopentylmethyl)-5-fluoro-6-(2-(trifluoromethyl)phenyl)-1H-indol-3-yl)-2,2,2-trifluoroethyl)cyclobutanesulfonamide (421)

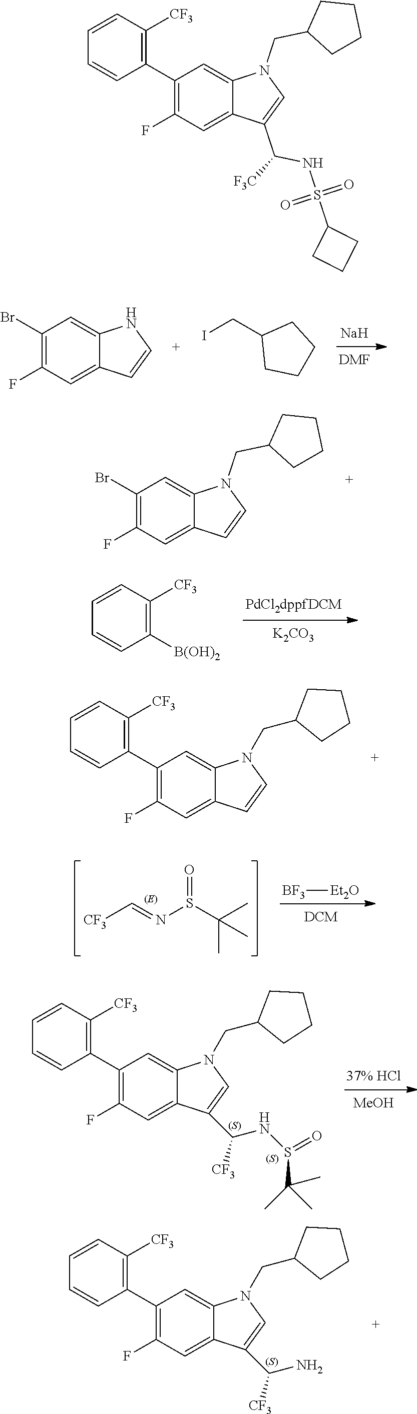

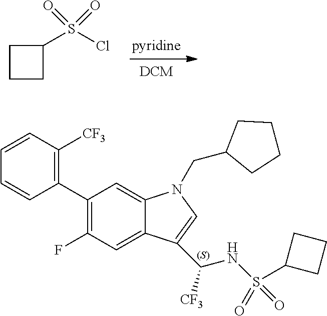

### Step 1: 6-bromo-1-(cyclopentylmethyl)-5-fluoro-1H-indole

The title compound was prepared following the same general protocol as described for Step 1, Example 1, using 6-bromo-5-fluoro-1H-indole and (iodomethyl)cyclopentane. ¹HNMR (CDCl₃, 400 MHz) δ 7.50 (d, J=5.7 Hz, 1H), 7.34 (d, J=9.2 Hz, 1H), 7.13 (d, J=3.2 Hz, 1H), 6.42 (dd, J1=3.1 Hz, J2=0.6 Hz, 1H), 3.98 (d, J=7.2 Hz, 2H), 2.35-2.44 (m, 1H), 1.64-1.74 (m, 4H), 1.5-1.62 (m, 2H), 1.22-1.30 (m, 2H).

### Step 2: 1-(cyclopentylmethyl)-5-fluoro-6-(2-(trifluoromethyl)phenyl)-1H-indole

The title compound was prepared following the same general protocol as described for Step 5, Example 1, using 6-bromo-1-(cyclopentylmethyl)-5-fluoro-1H-indole and (2-(trifluoromethyl)phenyl)boronic acid. ESI-MS (m/z): 362.2 [M+1]⁺.

### Step 3: (S)—N—((S)-1-(1-(cyclopentylmethyl)-5-fluoro-6-(2-(trifluoromethyl)phenyl)-1H-indol-3-yl)-2,2,2-trifluoroethyl)-2-methylpropane-2-sulfinamide

The title compound was prepared following the same general protocol as described for Step 3, Example 116, using 1-(cyclopentylmethyl)-5-fluoro-6-(2-(trifluoromethyl)phenyl)-1H-indole. ESI-MS (m/z): 563.2 [M+1]⁺.

### Step 4: (S)-1-(1-(cyclopentylmethyl)-5-fluoro-6-(2-(trifluoromethyl)phenyl)-1H-indol-3-yl)-2,2,2-trifluoroethan-1-amine

The title compound was prepared following the same general protocol as described for Step 4, Example 116, using (S)—N—((S)-1-(1-(cyclopentylmethyl)-5-fluoro-6-(2-(trifluoromethyl)phenyl)-1H-indol-3-yl)-2,2,2-trifluoroethyl)-2-methylpropane-2-sulfinamide. ESI-MS (m/z): 442.2 [M-NH₂]⁺.

### Step 5: (S)—N-(1-(1-(cyclopentylmethyl)-5-fluoro-6-(2-(trifluoromethyl)phenyl)-1H-indol-3-yl)-2,2,2-trifluoroethyl)cyclobutanesulfonamide

The title compound was prepared following the same general protocol as described for Step 5, Example 116, using (S)-1-(1-(cyclopentylmethyl)-5-fluoro-6-(2-(trifluoromethyl)phenyl)-1H-indol-3-yl)-2,2,2-trifluoroethan-1-amine. ESI-MS (m/z): 577.2 [M+1]⁺.

### Example 422: (S)—N-(1-(1-(cyclopentylmethyl)-5-fluoro-6-(2-(trifluoromethyl)phenyl)-1H-indol-3-yl)-2,2,2-trifluoroethyl)cyclopropanesulfonamide (422)

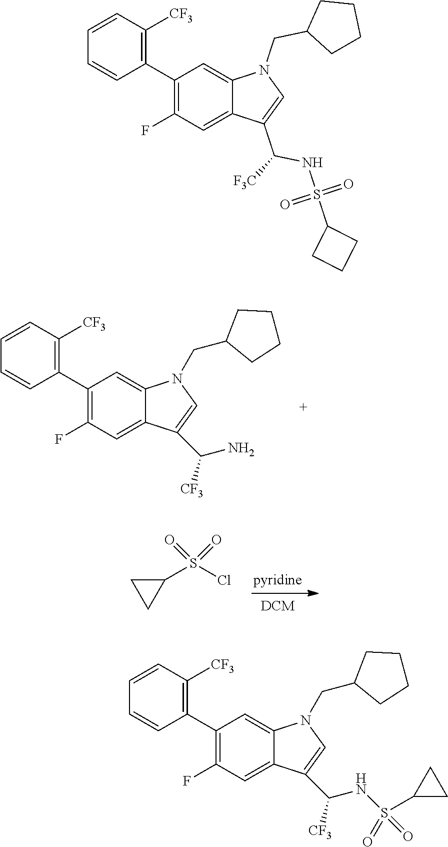 [SMILES: O=S(=O)(N[C@@H](c1cn(CC2CCCC2)c2cc(-c3ccccc3C(F)(F)F)c(F)cc12)C(F)(F)F)C1CCC1 ; N[C@@H](c1cn(CC2CCCC2)c2cc(-c3ccccc3C(F)(F)F)c(F)cc12)C(F)(F)F ; O=S(=O)(Cl)C1CC1 ; O=S(=O)(N[C@@H](c1cn(CC2CCCC2)c2cc(-c3ccccc3C(F)(F)F)c(F)cc12)C(F)(F)F)C1CC1]

The title compound was prepared following the same general protocol as described for Step 5, Example 116, using (S)-1-(1-(cyclopentylmethyl)-5-fluoro-6-(2-(trifluoromethyl)phenyl)-1H-indol-3-yl)-2,2,2-trifluoroethan-1-amine and cyclopropanesulfonyl chloride. ESI-MS (m/z): 563.2 [M+1]⁺.

### Example 423: N-(1-(5-fluoro-1-neopentyl-6-(2-(trifluoromethyl)phenyl)-1H-indol-3-yl)ethyl)cyclopropanesulfonamide (423)

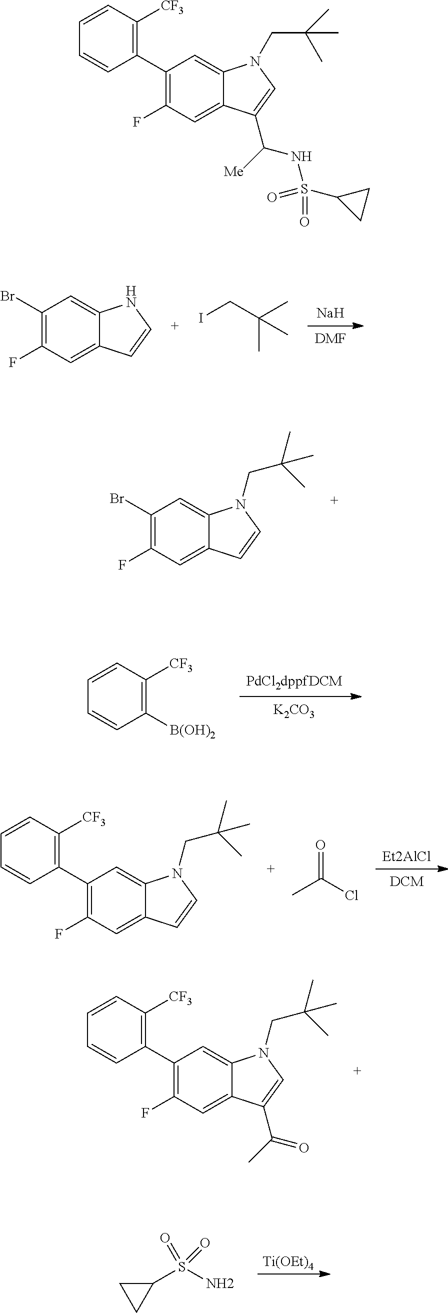

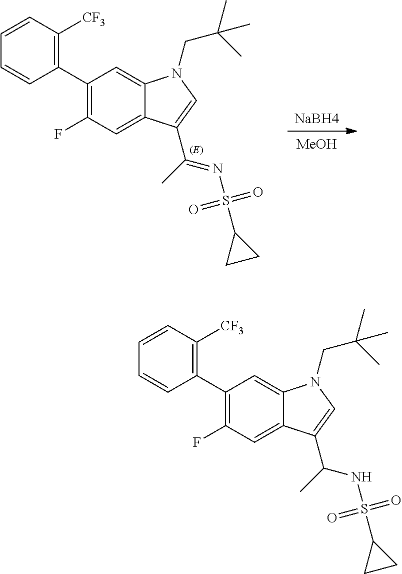 [SMILES: "MeOH" ; C/C(=N\S(=O)(=O)C1CC1)c1cn(CC(C)(C)C)c2cc(-c3ccccc3C(F)(F)F)c(F)cc12 ; "NaBH4" ; CC(NS(=O)(=O)C1CC1)c1cn(CC(C)(C)C)c2cc(-c3ccccc3C(F)(F)F)c(F)cc12]

### Step 1: 6-bromo-5-fluoro-1-neopentyl-1H-indole

The title compound was prepared following the same general protocol as described for Step 1, Example 1, using 6-bromo-5-fluoro-1H-indole and 1-iodo-2,2-dimethylpropane. The product was used in next step directly.

### Step 2: 5-fluoro-1-neopentyl-6-(2-(trifluoromethyl)phenyl)-1H-indole

The title compound was prepared following the same general protocol as described for Step 5, Example 1, using 6-bromo-5-fluoro-1-neopentyl-1H-indole and (2-(trifluoromethyl)-phenyl)boronic acid. ESI-MS (m/z): 350.2 [M+1]⁺.

### Step 3: 1-(5-fluoro-1-neopentyl-6-(2-(trifluoromethyl)phenyl)-1H-indol-3-yl)ethan-1-one

The title compound was prepared following the same general protocol as described for Step 1, Example 87, using 5-fluoro-1-neopentyl-6-(2-(trifluoromethyl)phenyl)-1H-indole and acetyl chloride. ESI-MS (m/z): 392.2 [M+1]⁺.

### Step 4: (E)-N-(1-(5-fluoro-1-neopentyl-6-(2-(trifluoromethyl)phenyl)-1H-indol-3-yl)ethylidene)cyclopropanesulfonamide

The title compound was prepared following the same general protocol as described for Step 4, Example 113, using 1-(5-fluoro-1-neopentyl-6-(2-(trifluoromethyl)phenyl)-1H-indol-3-yl)ethan-1-one and cyclopropanesulfonamide. ESI-MS (m/z): 495.2 [M+1]⁺.

### Step 5: N-(1-(5-fluoro-1-neopentyl-6-(2-(trifluoromethyl)phenyl)-1H-indol-3-yl)ethyl)cyclopropanesulfonamide

The title compound was prepared following the same general protocol as described for Step 3, Example 420 (Compound 420), using (E)-N-(1-(5-fluoro-1-neopentyl-6-(2-(trifluoromethyl)phenyl)-1H-indol-3-yl)ethylidene)cyclopropanesulfonamide. ESI-MS (m/z): 619.2 [M+Na]⁺.

### Example 424: N-(cyclopropyl(1-neopentyl-6-(2-(trifluoromethyl)phenyl)-1H-indol-3-yl)methyl)cyclobutanesulfonamide (424)

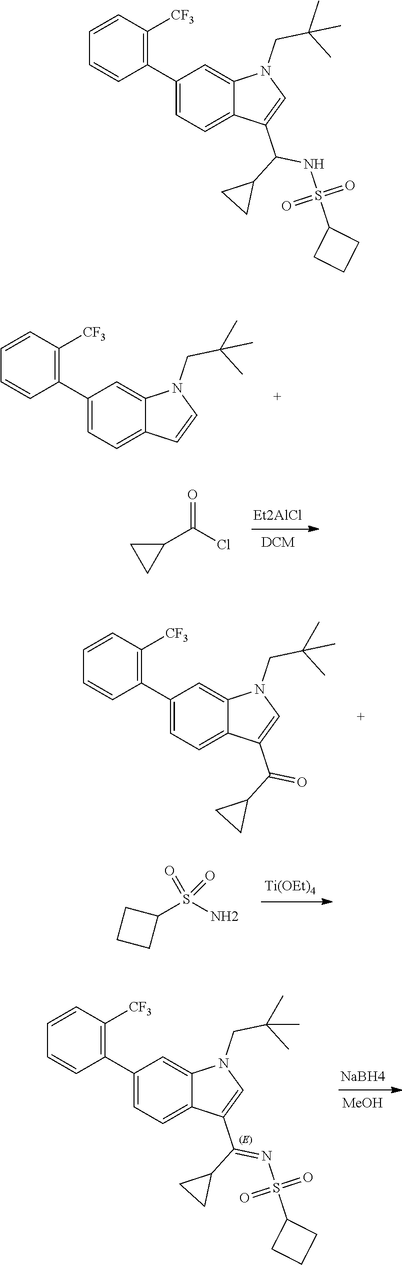

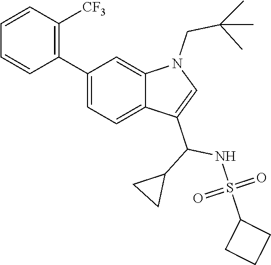 [SMILES: CC(C)(C)Cn1cc(C(NS(=O)(=O)C2CCC2)C2CC2)c2ccc(-c3ccccc3C(F)(F)F)cc21]

### Step 1: cyclopropyl(1-neopentyl-6-(2-(trifluoromethyl)phenyl)-1H-indol-3-yl)methanone

The title compound was prepared following the same general protocol as described for Step 1, Example 87, using 1-neopentyl-6-(2-(trifluoromethyl)phenyl)-1H-indole and cyclopropane-carbonyl chloride. ESI-MS (m/z): 400.2 [M+1]⁺. ¹HNMR (CDCl₃, 400 MHz) δ 8.39 (dd, J1=8.1 Hz, J2=0.6 Hz, 1H), 7.88 (s, 1H), 7.76 (d, J=7.4 Hz, 1H), 7.57 (t, J=7.4 Hz, 1H), 7.47 (t, J=7.4 Hz, 1H), 7.38 (d, J=7.5 Hz, 1H), 7.34 (s, 1H), 7.22 (dd, J1=7.9 Hz, J2=1.6 Hz, 1H), 3.96 (s, 2H), 2.40-2.50 (m, 1H), 1.24-1.30 (m, 2H), 1.04 (s, 9H), 0.90-1.00 (m, 2H).

### Step 2: (E)-N-(cyclopropyl(1-neopentyl-6-(2-(trifluoromethyl)phenyl)-1H-indol-3-yl)methylene)cyclobutanesulfonamide

The title compound was prepared following the same general protocol as described for Step 4, Example 113, using cyclopropyl(1-neopentyl-6-(2-(trifluoromethyl)phenyl)-1H-indol-3-yl)methanone and cyclobutanesulfonamide. ESI-MS (m/z): 517.2 [M+1]⁺.

### Step 3: N-(cyclopropyl(1-neopentyl-6-(2-(trifluoromethyl)phenyl)-1H-indol-3-yl)methyl)cyclobutanesulfonamide

The title compound was prepared following the same general protocol as described for Step 3, Example 420 (Compound 420), using (E)-N-(cyclopropyl(1-neopentyl-6-(2-(trifluoromethyl)phenyl)-1H-indol-3-yl)methylene)cyclobutanesulfonamide. ESI-MS (m/z): 519.2 [M+1]⁺.

### Example 425: N-(1-(1-neopentyl-6-(2-(trifluoromethyl)phenyl)-1H-indol-3-yl)propyl)cyclobutanesulfonamide (425)

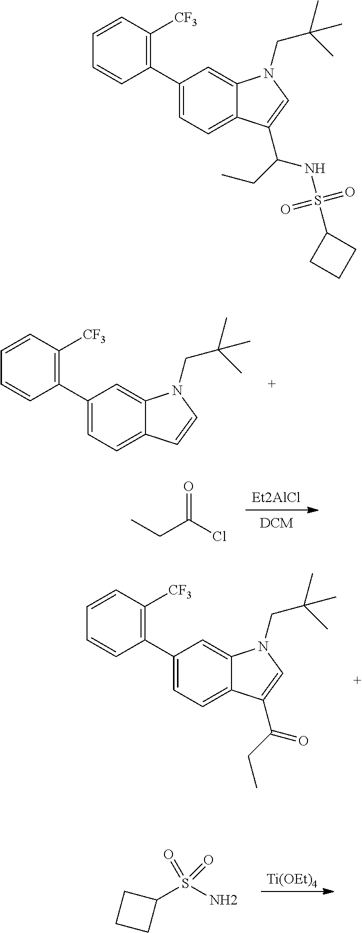

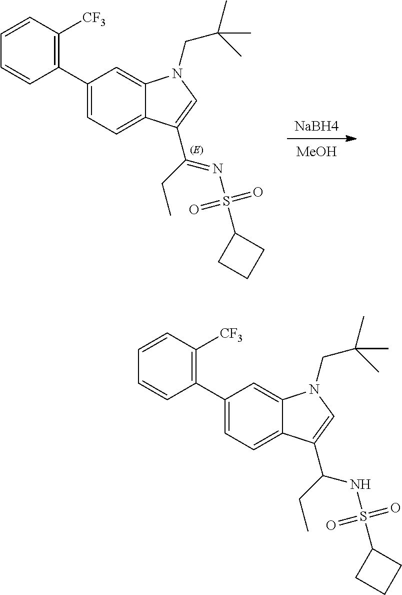 [SMILES: "MeOH" ; CC/C(=N\S(=O)(=O)C1CCC1)c1cn(CC(C)(C)C)c2cc(-c3ccccc3C(F)(F)F)ccc12 ; "NaBH4" ; CCC(NS(=O)(=O)C1CCC1)c1cn(CC(C)(C)C)c2cc(-c3ccccc3C(F)(F)F)ccc12]

### Step 1: 1-(1-neopentyl-6-(2-(trifluoromethyl)phenyl)-1H-indol-3-yl)propan-1-one

The title compound was prepared following the same general protocol as described for Step 1, Example 87, using 1-neopentyl-6-(2-(trifluoromethyl)phenyl)-1H-indole and propionyl chloride. ESI-MS (m/z): 388.2 [M+1]⁺. ¹HNMR (CDCl₃, 400 MHz) δ 8.39 (dd, J1=8.1 Hz, J2=0.6 Hz, 1H), 7.76 (d, J=7.4 Hz, 1H), 7.75 (s, 1H), 7.54 (t, J=7.4 Hz, 1H), 7.47 (t, J=7.4 Hz, 1H), 7.37 (d, J=7.5 Hz, 1H), 7.33 (s, 1H), 7.23 (dd, J1=7.9 Hz, J2=1.6 Hz, 1H), 3.94 (s, 2H), 2.93 (q, J=5.6 Hz, 1H), 1.29 (t, J=7.4 Hz, 3H), 1.02 (s, 9H).

### Step 2: (E)-N-(1-(1-neopentyl-6-(2-(trifluoromethyl)phenyl)-1H-indol-3-yl)propylidene)-cyclobutanesulfonamide

The title compound was prepared following the same general protocol as described for Step 4, Example 113, using 1-(1-neopentyl-6-(2-(trifluoromethyl)phenyl)-1H-indol-3-yl)propan-1-one and cyclobutanesulfonamide. ESI-MS (m/z): 505.2 [M+1]⁺.

### Step 3: N-(1-(1-neopentyl-6-(2-(trifluoromethyl)phenyl)-1H-indol-3-yl)propyl)-cyclobutanesulfonamide

The title compound was prepared following the same general protocol as described for Step 3, Example 420 (Compound 420), using (E)-N-(1-(1-neopentyl-6-(2-(trifluoromethyl)phenyl)-1H-indol-3-yl)propylidene)-cyclobutanesulfonamide. ESI-MS (m/z): 507.2 [M+1]⁺.

### Example 426: 2,2-dimethyl-N—((S)-2,2,2-trifluoro-1-(1-neopentyl-6-(2-(trifluoromethyl)phenyl)-1H-indol-3-yl)ethyl)cyclopropane-1-sulfonamide (426)

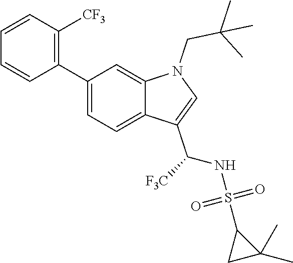 [SMILES: CC(C)(C)Cn1cc([C@H](NS(=O)(=O)C2CC2(C)C)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 2,2-dimethylcyclopropane-1-sulfonyl chloride. ¹HNMR (CDCl₃, 400 MHz) δ 7.76 (d, J=8.0 Hz, 1H), 7.71 (d, J=8.0 Hz, 1H), 7.56 (t, J=8.0 Hz, 1H), 7.47 (t, J=8.0 Hz, 1H), 7.37-7.32 (m, 2H), 7.22 (s, 1H), 7.15-7.12 (m, 1H), 5.50-5.39 (m, 1H), 5.00-4.94 (m, 1H), 3.89 (d, J=4.0 Hz, 2H), 2.29-2.20 (m, 1H), 2.00 (s, 1H), 1.61 (s, 1H), 1.57-1.30 (m, 2H), 1.19-1.10 (m, 2H), 0.98 (s, 9H), 0.88-0.79 (m, 1H), 0.68 (s, 1H)

### Example 427: (S)—N-(1-(6-cyclopentyl-1-neopentyl-1H-pyrrolo[2,3-b]pyridin-3-yl)-2,2,2-trifluoroethyl)cyclobutanesulfonamide (427)

### Step 1: (S)-1-(6-cyclopentenyl-1-neopentyl-1H-pyrrolo[2,3-b]pyridin-3-yl)-2,2,2-trifluoroethanamine

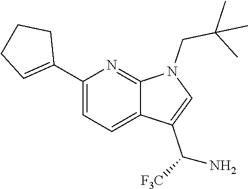 [SMILES: CC(C)(C)Cn1cc([C@H](N)C(F)(F)F)c2ccc(C3=CCCC3)nc21]

A mixture of (S)-1-(6-bromo-1-neopentyl-1H-pyrrolo[2,3-b]pyridin-3-yl)-2,2,2-trifluoroethanamine (345 mg, 947.27 μmol), 2-(cyclopenten-1-yl)-4,4,5,5-tetramethyl-1,3,2-dioxaborolane (275.77 mg, 1.42 mmol), Pd(dppf)Cl2 (138.62 mg, 189.45 μmol) and Cs2CO3 (617.28 mg, 1.89 mmol) in dioxane (12 mL) and Water (3 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na₂SO₄ and filtered. The filtrate was concentrated to give the crude product, which was purified by silica gel column (EA/PE=0-50%) to get (S)-1-(6-cyclopentenyl-1-neopentyl-1H-pyrrolo[2,3-b]pyridin-3-yl)-2,2,2-trifluoroethanamine (240 mg, 682.97 μmol, 72.10% yield) as yellow oil.

### Step 2: (S)-1-(6-cyclopentyl-1-neopentyl-1H-pyrrolo[2,3-b]pyridin-3-yl)-2,2,2-trifluoroethanamine

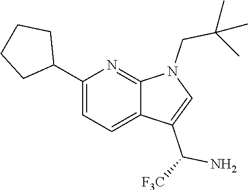 [SMILES: CC(C)(C)Cn1cc([C@H](N)C(F)(F)F)c2ccc(C3CCCC3)nc21]

A mixture of (S)-1-(6-cyclopentenyl-1-neopentyl-1H-pyrrolo[2,3-b]pyridin-3-yl)-2,2,2-trifluoroethanamine (240 mg, 682.97 μmol) and 10% Pd/C (42 mg) in Methanol (10 mL) was stirred at room temperature overnight under H2. The mixture was filtered, The filtrate was concentrated to get (S)-1-(6-cyclopentyl-1-neopentyl-1H-pyrrolo[2,3-b]pyridin-3-yl)-2,2,2-trifluoroethanamine (237 mg, 670.58 μmol, 98.19% yield) as yellow oil.

### Step 3: (S)—N-(1-(6-cyclopentyl-1-neopentyl-1H-pyrrolo[2,3-b]pyridin-3-yl)-2,2,2-trifluoroethyl)cyclobutanesulfonamide

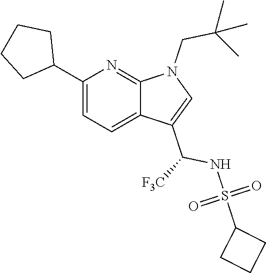 [SMILES: CC(C)(C)Cn1cc([C@H](NS(=O)(=O)C2CCC2)C(F)(F)F)c2ccc(C3CCCC3)nc21]

A mixture of (S)-1-(6-cyclopentyl-1-neopentyl-1H-pyrrolo[2,3-b]pyridin-3-yl)-2,2,2-trifluoroethanamine (237 mg, 670.58 μmol) and cyclobutanesulfonyl chloride (266.61 mg, 1.21 mmol) in anhydrous pyridine (0.5 mL) was stirred at 100° C. for 4 hours in the microwave. The mixture was added EtOAc and concentrated. The residue was purified by silica gel column to get the crude product, which was by prep-HPLC to get (S)—N-(1-(6-cyclopentyl-1-neopentyl-1H-pyrrolo[2,3-b]pyridin-3-yl)-2,2,2-trifluoroethyl)cyclobutanesulfonamide (20.30 mg, 43.05 μmol, 6.42% yield) as a white solid. ¹H NMR (400 MHz, CDCl₃) δ 7.89 (d, J=8.1 Hz, 1H), 7.21 (s, 1H), 7.00 (d, J=8.1 Hz, 1H), 5.28 (p, J=7.8 Hz, 1H), 5.04 (d, J=8.4 Hz, 1H), 4.13-4.02 (m, 2H), 3.74 (p, J=8.3 Hz, 1H), 3.25 (p, J=8.0 Hz, 1H), 2.42 (dt, J=17.9, 9.0 Hz, 2H), 2.22-2.00 (m, 4H), 1.95-1.80 (m, 6H), 1.69 (dd, J=7.0, 2.3 Hz, 2H), 0.97 (s, 9H). LCMS: 471.8 m/z [M+1]⁺.

### Example 428: (S)—N-(1-(6-cyclopentyl-5-fluoro-1-neopentyl-1H-indol-3-yl)-2,2,2-trifluoroethyl)cyclobutanesulfonamide (428)

### Step 1: (S)-1-(6-cyclopentenyl-5-fluoro-1-neopentyl-1H-indol-3-yl)-2,2,2-trifluoroethanamine

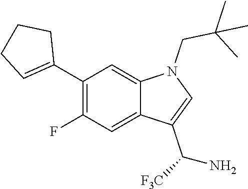 [SMILES: CC(C)(C)Cn1cc([C@H](N)C(F)(F)F)c2cc(F)c(C3=CCCC3)cc21]

A mixture of (S)-1-(6-bromo-5-fluoro-1-neopentyl-1H-indol-3-yl)-2,2,2-trifluoroethanamine (260 mg, 682.05 μmol), (S)-1-(6-bromo-5-fluoro-1-neopentyl-1H-indol-3-yl)-2,2,2-trifluoroethanamine (260 mg, 682.05 μmol), Pd(dppf)Cl2 (99.81 mg, 136.41 μmol) and Cs2CO3 (555.56 mg, 1.71 mmol) in dioxane (12 mL) and Water (3 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-33%) to get (S)-1-(6-cyclopentenyl-5-fluoro-1-neopentyl-1H-indol-3-yl)-2,2,2-trifluoroethanamine (246 mg, 667.73 μmol, 97.90% yield) as light-yellow oil.

### Step 2: The synthesis of (S)-1-(6-cyclopentyl-5-fluoro-1-neopentyl-1H-indol-3-yl)-2,2,2-trifluoroethanamine

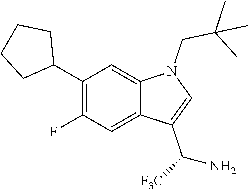 [SMILES: CC(C)(C)Cn1cc([C@H](N)C(F)(F)F)c2cc(F)c(C3CCCC3)cc21]

A mixture of (S)-1-(6-cyclopentenyl-5-fluoro-1-neopentyl-1H-indol-3-yl)-2,2,2-trifluoroethanamine (246 mg, 667.73 μmol) and 10% Pd/C (50 mg, 667.73 μmol) in Methanol (10 mL) was stirred at room temperature for 2.5 days under H₂. The mixture was filtered. The filtrate was concentrated to get oil, which was purified by prep-HPLC to get (S)-1-(6-cyclopentyl-5-fluoro-1-neopentyl-1H-indol-3-yl)-2,2,2-trifluoroethanamine (112 mg, 302.35 μmol, 45.28% yield) as a white solid.

### Step 3: (S)—N-(1-(6-cyclopentyl-5-fluoro-1-neopentyl-1H-indol-3-yl)-2,2,2-trifluoroethyl)cyclobutanesulfonamide

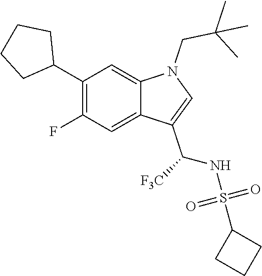 [SMILES: CC(C)(C)Cn1cc([C@H](NS(=O)(=O)C2CCC2)C(F)(F)F)c2cc(F)c(C3CCCC3)cc21]

A mixture of (S)-1-(6-cyclopentyl-5-fluoro-1-neopentyl-1H-indol-3-yl)-2,2,2-trifluoroethanamine (112 mg, 302.35 μmol) and cyclobutanesulfonyl chloride (146.92 mg, 665.18 μmol) in anhydrous pyridine (0.4 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(1-(6-cyclopentyl-5-fluoro-1-neopentyl-1H-indol-3-yl)-2,2,2-trifluoroethyl)cyclobutanesulfonamide (10.33 mg, 21.14 μmol, 6.99% yield) as a white solid. ¹H NMR (400 MHz, CDCl₃) δ 7.30 (d, J=11.0 Hz, 1H), 7.16 (d, J=5.8 Hz, 1H), 7.12 (s, 1H), 5.34-5.17 (m, 1H), 4.88 (d, J=8.1 Hz, 1H), 3.92-3.69 (m, 3H), 3.43-3.26 (m, 1H), 2.46 (dd, J=20.0, 10.0 Hz, 2H), 2.25-2.06 (m, 4H), 1.92 (dd, J=15.9, 8.8 Hz, 2H), 1.85 (d, J=15.1 Hz, 2H), 1.74 (dd, J=14.1, 7.2 Hz, 2H), 1.66 (d, J=17.8 Hz, 2H), 0.97 (s, 9H). LCMS: 488.8 m/z [M+1]⁺.

### Example 429: (R)—N-(2-fluoro-1-(1-neopentyl-6-(2-(trifluoromethyl)phenyl)-1H-indol-3-yl)ethyl)cyclobutanesulfonamide (429)

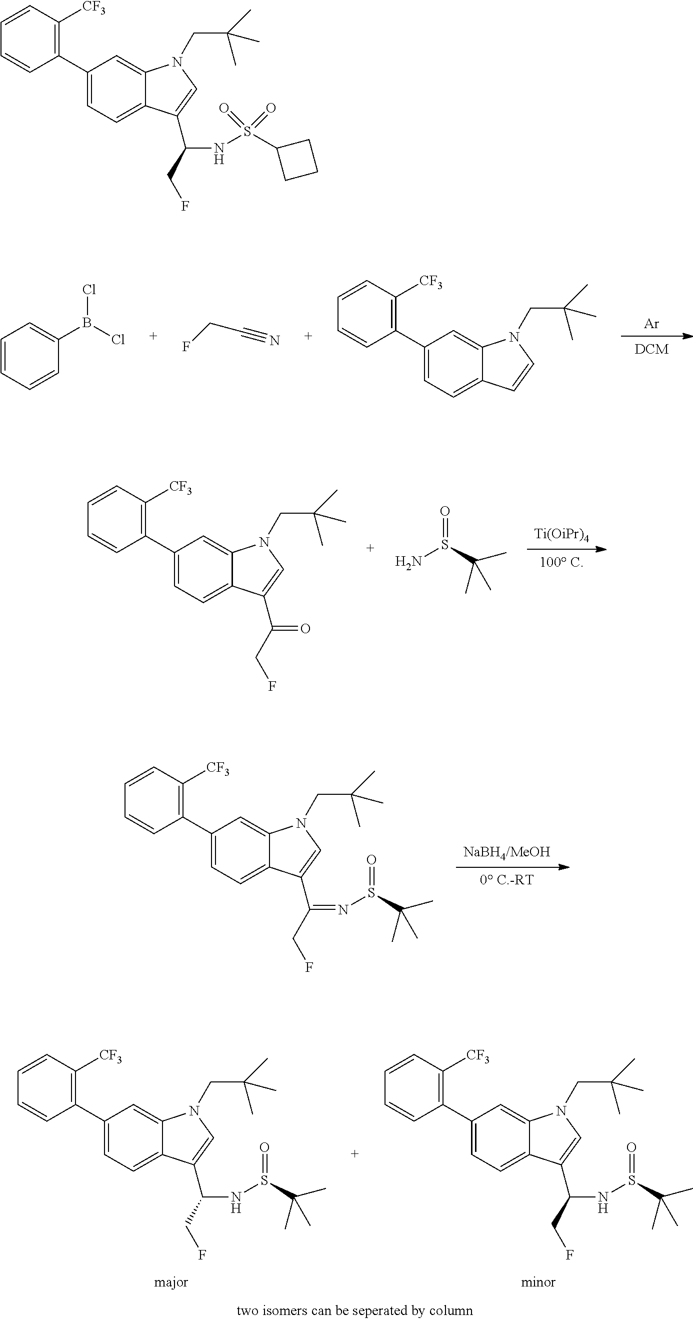

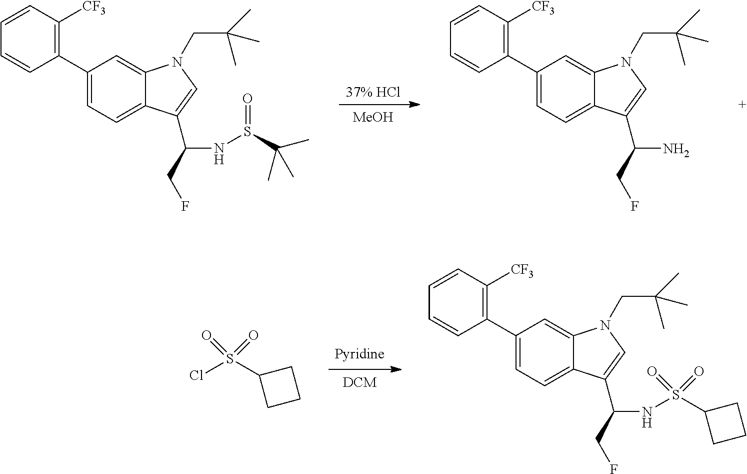

### Step 1: 2-fluoro-1-(1-neopentyl-6-(2-(trifluoromethyl)phenyl)-1H-indol-3-yl)ethan-1-one

PhBCl₂ (3 mmol) was added to a solution of 2-fluoroacetonitrile (2.4 mmol) in DCM (20 mL) at room temperature under argon. After stirring for 15 mins, the indole (2 mmol) was added. The resulting solution was stirred for additional 3 hours and quenched with Na₂CO₃ solution. The layers were separated and the aqueous layer was extracted with DCM (2×30 mL). The combined organics were washed with brine (2×30 mL), dried (Na₂SO₄) and filtered. The solvent was removed under reduced pressure and the residue was purified by combi-flash to obtain the title compound (0.4 g) as an oil. ESI-MS (m/z): 392.2 [M+1]⁺. ¹HNMR (CDCl3, 400 MHz) δ 8.42 (dd, J1=8.1 Hz, J2=0.6 Hz, 1H), 8.06 (d, J=2.1 Hz, 1H), 7.77 (d, J=7.4 Hz, 1H), 7.58 (t, J=7.2 Hz, 1H), 7.49 (t, J=7.4 Hz, 1H), 7.38 (d, J=7.7 Hz, 1H), 7.39 (s, 1H), 7.28 (s, 1H), 5.32 (s, 1H), 5.20 (s, 1H), 3.96 (s, 2H), 1.04 (s, 9H).

### Step 2: (S, E)-N-(2-fluoro-1-(1-neopentyl-6-(2-(trifluoromethyl)phenyl)-1H-indol-3-yl) ethylidene)-2-methylpropane-2-sulfinamide

The title compound was prepared following the same general protocol as described for Step 4, Example 113, using 1-(1-neopentyl-6-(2-(trifluoromethyl)phenyl)-1H-indol-3-yl)propan-1-one. ESI-MS (m/z): 495.2 [M+1]⁺.

### Step 3: (S)—N—((S)-2-fluoro-1-(1-neopentyl-6-(2-(trifluoromethyl)phenyl)-1H-indol-3-yl)ethyl)-2-methylpropane-2-sulfinamide

The title compound was prepared following the same general protocol as described for Step 3, Example 420 (Compound 420), using (S, E)-N-(2-fluoro-1-(1-neopentyl-6-(2-(trifluoromethyl)phenyl)-1H-indol-3-yl)ethylidene)-2-methylpropane-2-sulfinamide. The two isomers can be separated by silica gel column. ESI-MS (m/z): 497.2 [M+1]⁺.

### Step 4: (R)-2-fluoro-1-(1-neopentyl-6-(2-(trifluoromethyl)phenyl)-1H-indol-3-yl)ethan-1-amine

The title compound was prepared following the same general protocol as described for Step 4, Example 116, using (R)—N—((S)-2-fluoro-1-(1-neopentyl-6-(2-(trifluoromethyl)phenyl)-1H-indol-3-yl)ethyl)-2-methylpropane-2-sulfinamide. ESI-MS (m/z): 376.2 [M-NH₂]⁺.

### Step 5: (R)—N-(2-fluoro-1-(1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2-fluoro-1-(1-neopentyl-6-(2-(trifluoromethyl)phenyl)-1H-indol-3-yl)ethan-1-amine and cyclobutanesulfonyl chloride. ESI-MS (m/z): 511.2 [M+1]⁺.

### Example 430: ethyl (S)-2-(cyclopropanesulfonamido)-2-(1-neopentyl-6-(2-(trifluoromethyl)phenyl)-1H-indol-3-yl)acetate (430)

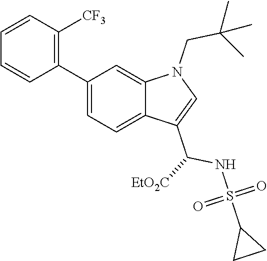 [SMILES: CCOC(=O)[C@@H](NS(=O)(=O)C1CC1)c1cn(CC(C)(C)C)c2cc(-c3ccccc3C(F)(F)F)ccc12]

### Step1: ethyl (S)-2-(((S)-tert-butylsulfinyl)amino)-2-(1-neopentyl-6-(2-(trifluoromethyl)phenyl)-1H-indol-3-yl)acetate

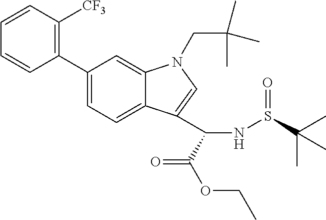 [SMILES: CCOC(=O)[C@@H](N[S@@](=O)C(C)(C)C)c1cn(CC(C)(C)C)c2cc(-c3ccccc3C(F)(F)F)ccc12]

To a vial of anhydrous Cu(OTf)₂ (0.112 g, 0.311 mmol) under argon at rt was added ethyl (S)-2-((tert-butylsulfinyl)imino)acetate (0.426 g, 2.075 mmol) in DCM (8 mL), followed by addition of 1-neopentyl-6-(2-(trifluoromethyl)phenyl)-1H-indole (0.825 g, 1.038 mmol). The mixture was continued to stir for 2 h at rt. The mixture was quenched by addition of saturated NH₄Cl and diluted with EtOAc. The layers were separated and the aqueous layer was extracted with EtOAc (2×). The organic layers were combined and concentrated, the crude was purified by silica gel to obtain the title compound. ¹HNMR (CDCl₃, 400 MHz) δ 7.75 (d, J=8.0 Hz, 1H), 7.63 (d, J=8.0 Hz, 1H), 7.54 (t, J=8.0 Hz, 1H), 7.46 (t, J=8.0 Hz, 1H), 7.38 (d, J=8.0 Hz, 1H), 7.28 (s, 1H), 7.12 (s, 1H), 7.04 (d, J=8.0 Hz, 1H), 5.35 (d, J=8.0 Hz, 1H), 4.51 (d, J=4.0 Hz, 1H), 4.29-4.18 (m, 2H), 3.86 (s, 2H), 1.27-1.20 (m, 12H), 0.98 (s, 9H)

### Step 2 ethyl (S)-2-amino-2-(1-neopentyl-6-(2-(trifluoromethyl)phenyl)-1H-indol-3-yl)acetate

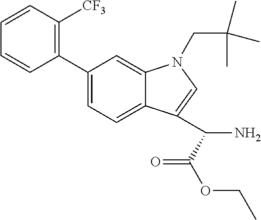 [SMILES: CCOC(=O)[C@@H](N)c1cn(CC(C)(C)C)c2cc(-c3ccccc3C(F)(F)F)ccc12]

The title compound was prepared following the same general protocol as described for Step 4, Example 116, using ethyl (S)-2-(((S)-tert-butylsulfinyl)amino)-2-(1-neopentyl-6-(2-(trifluoromethyl)phenyl)-1H-indol-3-yl)acetate.

### Step 3 ethyl (S)-2-(cyclopropanesulfonamido)-2-(1-neopentyl-6-(2-(trifluoromethyl)phenyl)-1H-indol-3-yl)acetate

The title compound was prepared following the same general protocol as described for Step 5, Example 116, using ethyl (S)-2-amino-2-(1-neopentyl-6-(2-(trifluoromethyl)phenyl)-1H-indol-3-yl)acetate. ¹HNMR (CDCl₃, 400 MHz) δ 7.76-7.73 (m, 2H), 7.56 (t, J=8.0 Hz, 1H), 7.47 (t, J=8.0 Hz, 1H), 7.37 (d, J=8.0 Hz, 1H), 7.29 (s, 1H), 7.14 (s, 1H), 7.09 (d, J=8.0 Hz, 1H), 5.49 (d, J=8.0 Hz, 1H), 5.38 (d, J=8.0 Hz, 1H), 4.32-4.21 (m, 2H), 3.87 (s, 2H), 2.24-2.18 (m, 1H), 1.27-1.22 (m, 3H), 1.18-1.14 (m, 2H), 0.98 (s, 9H), 0.77-0.75 (m, 2H)

### Example 431: (S)-2-(cyclopropanesulfonamido)-2-(1-neopentyl-6-(2-(trifluoromethyl)phenyl)-1H-indol-3-yl)acetic acid (431)

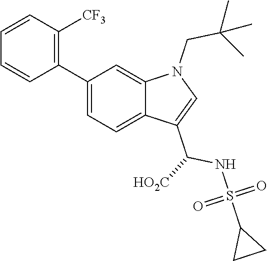 [SMILES: CC(C)(C)Cn1cc([C@H](NS(=O)(=O)C2CC2)C(=O)O)c2ccc(-c3ccccc3C(F)(F)F)cc21]

To ethyl (S)-2-(cyclopropanesulfonamido)-2-(1-neopentyl-6-(2-(trifluoromethyl)phenyl)-1H-indol-3-yl)acetate (0.183 g, 0.339 mmol) in THF (1 mL) was added NaOH (3M, 0.23 mL, 0.69 mmol) at rt. The reaction was monitored by anal. HPLC for completion. The crude was purified by prep. HPLC to obtain the title compound. ESI-MS (m/z): 508.72 [M+1]⁺.

### Example 432: (S)—N-(2-hydroxy-1-(1-neopentyl-6-(2-(trifluoromethyl)phenyl)-1H-indol-3-yl)ethyl)cyclopropanesulfonamide (432)

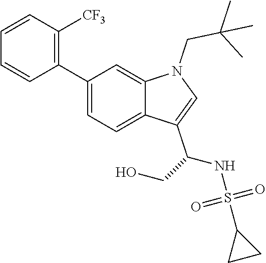 [SMILES: CC(C)(C)Cn1cc([C@@H](CO)NS(=O)(=O)C2CC2)c2ccc(-c3ccccc3C(F)(F)F)cc21]

To a solution of (S)-2-(cyclopropanesulfonamido)-2-(1-neopentyl-6-(2-(trifluoromethyl)phenyl)-1H-indol-3-yl)acetic acid (0.031 g, 0.06 mmol) in THF was added BH₃ (2M in THF, 0.1 mL, 0.2 mmol). The reaction was monitored by anal. HPLC for completion. When complete, the reaction was quenched with MeOH and concentrated in vacuo. The crude was purified by prep. HPLC to obtain the title compound. ESI-MS (m/z): 494.75 [M+1]⁺.

### Example 433: N-(1-(1-neopentyl-6-(2-(trifluoromethyl)phenyl)-1H-indol-3-yl)ethyl)bicyclo[1.1.1]pentane-1-sulfonamide (433)

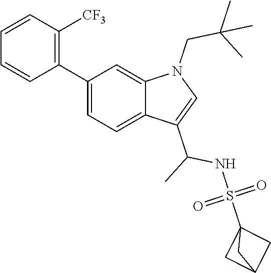 [SMILES: CC(NS(=O)(=O)C12CC(C1)C2)c1cn(CC(C)(C)C)c2cc(-c3ccccc3C(F)(F)F)ccc12]

### Step 1: N-(1-(1-neopentyl-6-(2-(trifluoromethyl)phenyl)-1H-indol-3-yl)ethylidene)bicyclo[1.1.1]pentane-1-sulfonamide

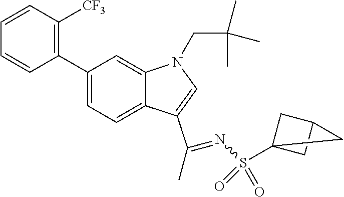 [SMILES: CC(=NS(=O)(=O)C12CC(C1)C2)c1cn(CC(C)(C)C)c2cc(-c3ccccc3C(F)(F)F)ccc12]

The title compound was prepared following the same general protocol as described for Step 4, Example 113, using 1-(1-neopentyl-6-(2-(trifluoromethyl)phenyl)-1H-indol-3-yl)ethan-1-one and bicyclo[1.1.1]pentane-1-sulfonamide. HNMR (CDCl₃, 400 MHz) δ 8.37 (d, J=8.0 Hz, 1H), 8.10 (s, 1H), 7.80 (d, J=8.0 Hz, 1H), 7.65 (t, J=8.0 Hz, 1H), 7.55 (t, J=8.0 Hz, 1H), 7.51 (s, 1H), 7.39 (d, J=8.0 Hz, 1H), 7.24 (d, J=8.0 Hz, 1H), 4.02 (s, 2H), 2.82 (s, 3H), 2.28 (s, 5H), 2.12 (s, 2H), 0.96 (s, 9H)

### Step 2: N-(1-(1-neopentyl-6-(2-(trifluoromethyl)phenyl)-1H-indol-3-yl)ethyl)bicyclo[1.1.1]pentane-1-sulfonamide

The title compound was prepared following the same general protocol as described for Step 3, Example 1, using N-(1-(1-neopentyl-6-(2-(trifluoromethyl)phenyl)-1H-indol-3-yl)ethylidene)bicyclo[1.1.1]pentane-1-sulfonamide. ¹HNMR (CDCl₃, 400 MHz) δ 7.76 (d, J=8.0 Hz, 1H), 7.72 (d, J=8.0 Hz, 1H), 7.56 (t, J=8.0 Hz, 1H), 7.47 (t, J=8.0 Hz, 1H), 7.39 (d, J=8.0 Hz, 1H), 7.30 (s, 1H), 7.08 (d, J=8.0 Hz, 1H), 7.06 (s, 1H), 5.00 (q, J=8.0 Hz, 1H), 4.40 (d, J=8.0 Hz, 1H), 3.85 (s, 2H), 2.59 (s, 1H), 2.10-2.01 (m, 6H), 1.75 (d, J=8.0 Hz, 3H), 0.98 (s, 9H)

### Example 434: 1-[[3-[(1S)-1-amino-2,2,2-trifluoro-ethyl]-5-fluoro-6-[2-(trifluoromethyl)phenyl]indol-1-yl]methyl]cyclopropanecarbonitrile (434)

### Step 1: 6-bromo-1-tetrahydropyran-4-yl-indole

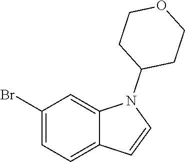 [SMILES: Brc1ccc2ccn(C3CCOCC3)c2c1]

A mixture of 6-bromo-1H-indole (1.27 g, 6.48 mmol), tetrahydropyran-4-yl methanesulfonate (1.06 g, 5.89 mmol) and Cs2CO₃ (3.84 g, 11.78 mmol) in anhydrous DMF (15 mL) was stirred for 64 h at 80° C. (70% of material wasn't reacted) the reaction mixture was cooled to room temperature, added water and ethyl acetate, stirred and separated out the organic phase. the aqueous phase was extracted with ethyl acetate twice. The combined organic phases were washed with brine, dried with anhydrous Na₂SO₄ and filtered. The filtrate was concentrated to give brown oil, which was purified by prep-HPLC to get 6-bromo-1-tetrahydropyran-4-yl-indole (240 mg, 856.65 μmol, 14.55% yield) as light-yellow solid.

### Step 2: (S)—N—((S)-1-(6-bromo-1-(tetrahydro-2H-pyran-4-yl)-1H-pyrrolo[2,3-b]pyridin-3-yl)-2,2,2-trifluoroethyl)-2-methylpropane-2-sulfinamide

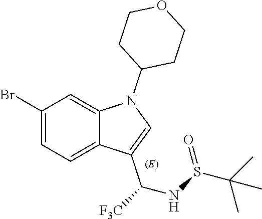 [SMILES: CC(C)(C)[S@](=O)N[C@@H](c1cn(C2CCOCC2)c2cc(Br)ccc12)C(F)(F)F]

To a stirred solution of 6-bromo-1-tetrahydropyran-4-yl-indole (240 mg, 856.65 μmol) in anhydrous CH₂Cl₂ (3 mL) was added BF₃·OEt₂ (218.85 mg, 1.54 mmol, 190.31 μL) at 0° C. under Nitrogen, followed by addition the CH₂Cl₂ mixture of imine (18 mL). The resulting mixture was stirred at room temperature for 2 h under nitrogen. The mixture was added water, stirred and separated organic phase, the aqueous phase was extracted with CH₂Cl₂ once. The combined organic phases were washed with brine and filtered. The filtrate was concentrated to get (S)—N—((S)-1-(6-bromo-1-(tetrahydro-2H-pyran-4-yl)-1H-pyrrolo[2,3-b]pyridin-3-yl)-2,2,2-trifluoroethyl)-2-methylpropane-2-sulfinamide as yellow oil, which was used directly next step.

### Step 3: (S)-1-(6-bromo-1-(tetrahydro-2H-pyran-4-yl)-1H-indol-3-yl)-2,2,2-trifluoroethanamine

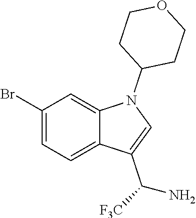 [SMILES: N[C@@H](c1cn(C2CCOCC2)c2cc(Br)ccc12)C(F)(F)F]

A mixture of (S)—N—((S)-1-(6-bromo-1-(tetrahydro-2H-pyran-4-yl)-1H-pyrrolo[2,3-b]pyridin-3-yl)-2,2,2-trifluoroethyl)-2-methylpropane-2-sulfinamide (413.21 mg, 856.65 μmol) in 4M HCl(g)/methanol (10 mL) was stirred at room temperature for 2 hours, the mixture was concentrated, the residue was basified to pH=8 with Sat. NaHCO3, the aqueous phase was extracted with EtOAc twice, the combined organic phases were washed with brine, dried with anhydrous Na2SO4 and filtered. The filtrate was concentrated to give the crude product, which was purified by silica gel column (EA/PE=0:1 to 1:1) to get (S)-1-(6-bromo-1-(tetrahydro-2H-pyran-4-yl)-1H-indol-3-yl)-2,2,2-trifluoroethanamine (320 mg, 848.36 μmol, 99.03% yield) as yellow oil.

### Step 4: (S)-2-(3-(1-amino-2,2,2-trifluoroethyl)-1-(tetrahydro-2H-pyran-4-yl)-1H-indol-6-yl)-3-fluorobenzamide

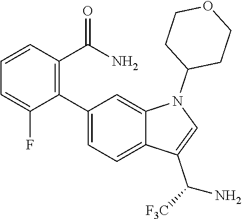 [SMILES: NC(=O)c1cccc(F)c1-c1ccc2c([C@H](N)C(F)(F)F)cn(C3CCOCC3)c2c1]

A mixture of (S)-1-(6-bromo-1-(tetrahydro-2H-pyran-4-yl)-1H-indol-3-yl)-2,2,2-trifluoroethanamine (320 mg, 848.36 μmol), (2-cyano-6-fluoro-phenyl)boronic acid (209.88 mg, 1.27 mmol), Pd(dppf)Cl₂ (124.15 mg, 169.67 μmol) and Cs₂CO₃ (691.03 mg, 2.12 mmol) in dioxane (12 mL) and water (3 mL) was stirred at 80° C. overnight under Nitrogen. The material was consumed completely. It was purified by silica gel column (EA/PE=0:1 to 1:1) to get (S)-2-(3-(1-amino-2,2,2-trifluoroethyl)-1-(tetrahydro-2H-pyran-4-yl)-1H-indol-6-yl)-3-fluorobenzamide (180 mg, 413.40 μmol, 48.73% yield).

### Step 5: 1-[[3-[(1S)-1-amino-2,2,2-trifluoro-ethyl]-5-fluoro-6-[2-(trifluoromethyl)phenyl]indol-1-yl]methyl]cyclopropanecarbonitrile

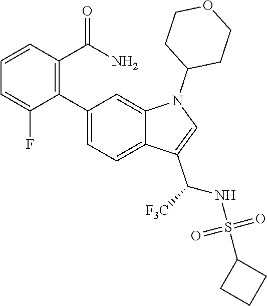 [SMILES: NC(=O)c1cccc(F)c1-c1ccc2c([C@H](NS(=O)(=O)C3CCC3)C(F)(F)F)cn(C3CCOCC3)c2c1]

A mixture of (S)-2-(3-(1-amino-2,2,2-trifluoroethyl)-1-(tetrahydro-2H-pyran-4-yl)-1H-indol-6-yl)-3-fluorobenzamide (180 mg, 413.40 μmol) and cyclobutanesulfonyl chloride (136.97 mg, 620.10 μmol) in anhydrous pyridine (0.4 mL) was stirred at 100° C. for 4 hours in the microwave. The mixture was added EtOAc and concentrated to get black oil, which was purified by silica gel column (MeOH/EtOAc=0:1 to 1:5) to get brown oil. It was purified by prep-HPLC to get (S)-2-(3-(1-(cyclobutanesulfonamido)-2,2,2-trifluoroethyl)-1-(tetrahydro-2H-pyran-4-yl)-1H-indol-6-yl)-3-fluorobenzamide (8.29 mg, 14.98 μmol, 3.62% yield) as a white solid. ¹H NMR (400 MHz, CDCl₃) δ 7.83 (d, J=8.2 Hz, 1H), 7.63 (d, J=7.2 Hz, 1H), 7.46 (d, J=3.4 Hz, 1H), 7.46-7.40 (m, 1H), 7.39 (s, 1H), 7.28 (dd, J=9.5, 1.2 Hz, 1H), 7.25 (dd, J=2.9, 1.3 Hz, 1H), 5.76 (s, 1H), 5.51-5.36 (m, 1H), 5.22 (s, 1H), 5.06 (d, J=8.3 Hz, 1H), 4.41 (ddd, J=16.0, 11.4, 4.4 Hz, 1H), 4.15-4.06 (m, 1H), 4.05-3.97 (m, 1H), 3.91 (p, J=8.3 Hz, 1H), 3.53 (tdd, J=11.4, 8.6, 2.6 Hz, 2H), 2.62-2.43 (m, 2H), 2.41-2.23 (m, 2H), 2.13-1.82 (m, 6H). LCMS: 571.2 m/z [M+18]⁺.

### Example 435: (S)—N-(2,2-difluoro-1-(5-fluoro-1-neopentyl-6-(2-(trifluoromethyl)phenyl)-1H-indol-3-yl)ethyl)cyclobutanesulfonamide (435)

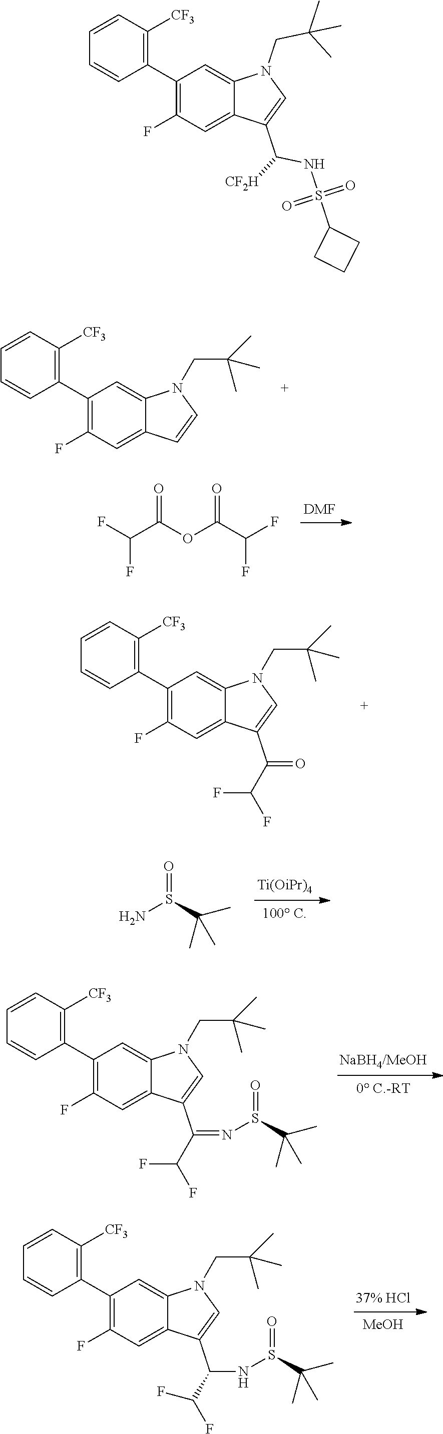

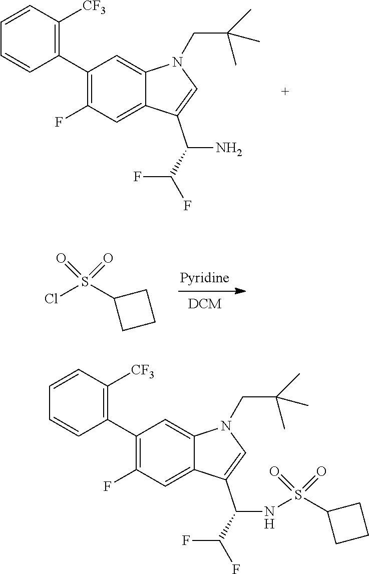

### Step 1: 2,2-difluoro-1-(5-fluoro-1-neopentyl-6-(2-(trifluoromethyl)phenyl)-1H-indol-3-yl)ethan-1-one

The title compound was prepared following the same general protocol as described for Step 1, Example 128, using 5-fluoro-1-neopentyl-6-(2-(trifluoromethyl)phenyl)-1H-indole and 2,2-difluoroacetic anhydride. ESI-MS (m/z): 428.1 [M+1]⁺.

### Step 2: (S, E)-N-(2,2-difluoro-1-(5-fluoro-1-neopentyl-6-(2-(trifluoromethyl)phenyl)-1H-indol-3-yl)ethylidene)-2-methylpropane-2-sulfinamide

The title compound was prepared following the same general protocol as described for Step 4, Example 113, using 2,2-difluoro-1-(5-fluoro-1-neopentyl-6-(2-(trifluoromethyl)phenyl)-1H-indol-3-yl)ethan-1-one. ESI-MS (m/z): 531.2 [M+1]⁺.

### Step 3: (S)—N—((S)-2,2-difluoro-1-(5-fluoro-1-neopentyl-6-(2-(trifluoromethyl)phenyl)-1H-indol-3-yl)ethyl)-2-methylpropane-2-sulfinamide

The title compound was prepared following the same general protocol as described for Step 3, Example 420 (Compound 420), using (S,E)-N-(2,2-difluoro-1-(5-fluoro-1-neopentyl-6-(2-(trifluoromethyl)phenyl)-1H-indol-3-yl)ethylidene)-2-methylpropane-2-sulfinamide. ESI-MS (m/z): 533.2 [M+1]⁺.

### Step 4: (S)-2,2-difluoro-1-(5-fluoro-1-neopentyl-6-(2-(trifluoromethyl)phenyl)-1H-indol-3-yl)ethan-1-amine

The title compound was prepared following the same general protocol as described for Step 4, Example 116, using (S)—N—((S)-2,2-difluoro-1-(5-fluoro-1-neopentyl-6-(2-(trifluoromethyl)-phenyl)-1H-indol-3-yl)ethyl)-2-methylpropane-2-sulfinamide. ESI-MS (m/z): 412.2 [M-NH₂]⁺.

### Step 5: (S)—N-(2,2-difluoro-1-(5-fluoro-1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2,2-difluoro-1-(5-fluoro-1-neopentyl-6-(2-(trifluoromethyl)phenyl)-1H-indol-3-yl)ethan-1-amine and cyclobutanesulfonyl chloride. ESI-MS (m/z): 547.2 [M+1]⁺.

### Example 436: (S)—N-(2-fluoro-1-(5-fluoro-1-neopentyl-6-(2-(trifluoromethyl)phenyl)-1H-indol-3-yl)ethyl)cyclobutanesulfonamide (436)

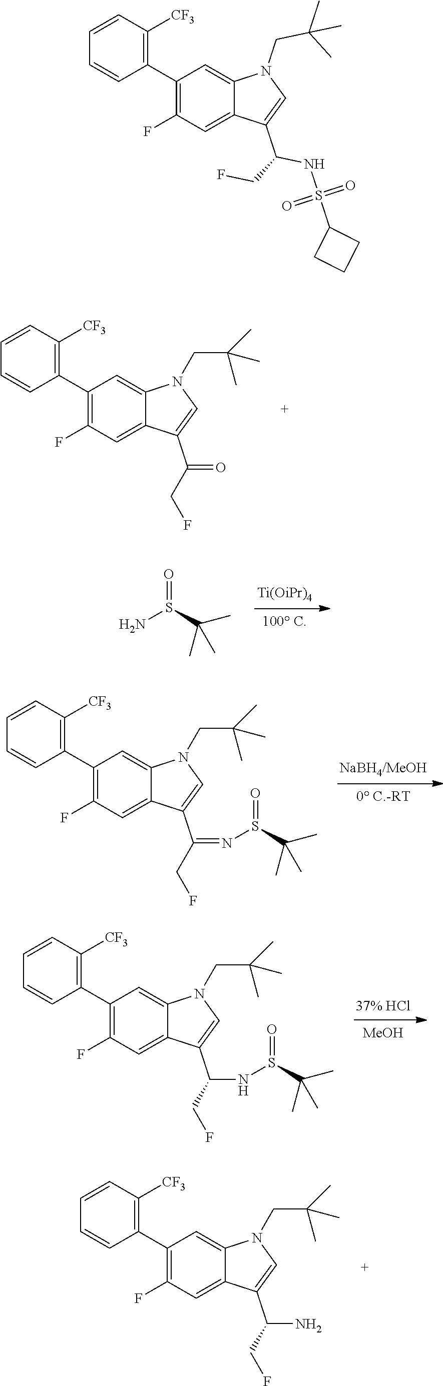

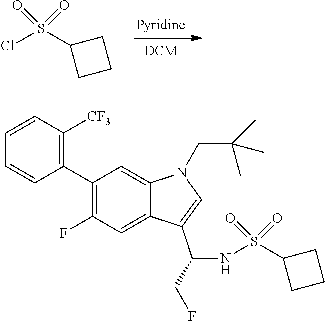

### Step 1: (S, E)-N-(2-fluoro-1-(1-neopentyl-6-(2-(trifluoromethyl)phenyl)-1H-indol-3-yl)ethylidene)-2-methylpropane-2-sulfinamide

The title compound was prepared following the same general protocol as described for Step 4, Example 113, using 1-(1-neopentyl-6-(2-(trifluoromethyl)phenyl)-1H-indol-3-yl)propan-1-one and (S)-2-methylpropane-2-sulfinamide. ESI-MS (m/z): 495.2 [M+1]⁺.

### Step 2: (S)—N—((S)-2-fluoro-1-(1-neopentyl-6-(2-(trifluoromethyl)phenyl)-1H-indol-3-yl)ethyl)-2-methylpropane-2-sulfinamide

The title compound was prepared following the same general protocol as described for Step 3, Example 420 (Compound 420), using (S,E)-N-(2-fluoro-1-(1-neopentyl-6-(2-(trifluoromethyl)phenyl)-1H-indol-3-yl)ethylidene)-2-methylpropane-2-sulfinamide. ESI-MS (m/z): 497.2 [M+1]⁺.

### Step 3: (S)-2-fluoro-1-(1-neopentyl-6-(2-(trifluoromethyl)phenyl)-1H-indol-3-yl)ethan-1-amine

The title compound was prepared following the same general protocol as described for Step 4, Example 116, using (S)—N—((S)-2-fluoro-1-(1-neopentyl-6-(2-(trifluoromethyl)phenyl)-1H-indol-3-yl)ethyl)-2-methylpropane-2-sulfinamide. ESI-MS (m/z): 376.2 [M-NH₂]⁺.

### Step 5: (S)—N-(2-fluoro-1-(1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2-fluoro-1-(1-neopentyl-6-(2-(trifluoromethyl)phenyl)-1H-indol-3-yl)ethan-1-amine and cyclobutanesulfonyl chloride. ESI-MS (m/z): 511.2 [M+1]⁺.

### Example 437: (S)-3-fluoro-N-(2,2,2-trifluoro-1-(1-neopentyl-6-(2-(trifluoromethyl)phenyl)-1H-indol-3-yl)ethyl)cyclobutane-1-sulfonamide (437)

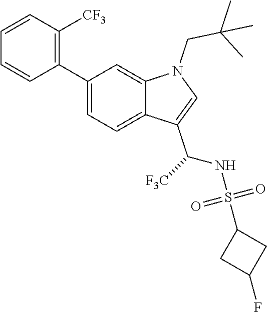 [SMILES: CC(C)(C)Cn1cc([C@H](NS(=O)(=O)C2CC(F)C2)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 3-fluorocyclobutane-1-sulfonyl chloride. ¹HNMR (CDCl₃, 400 MHz) δ 7.76 (d, J=8.0 Hz, 1H), 7.65 (d, J=8.0 Hz, 1H), 7.57 (t, J=8.0 Hz, 1H), 7.48 (t, J=8.0 Hz, 1H), 7.40-7.33 (m, 2H), 7.29-7.22 (m, 1H), 7.13 (d, J=8.0 Hz, 1H), 5.42-5.13 (m, 2H), 4.99-4.71 (m, 1H), 3.99-3.83 (m, 2H), 3.35-2.72 (m, 5H), 0.99 (s, 9H)

### Example 438: (S)—N-(2,2,2-trifluoro-1-(1-propyl-6-(2-(trifluoromethyl)phenyl)-1H-indol-3-yl)ethyl)cyclobutanesulfonamide (438)

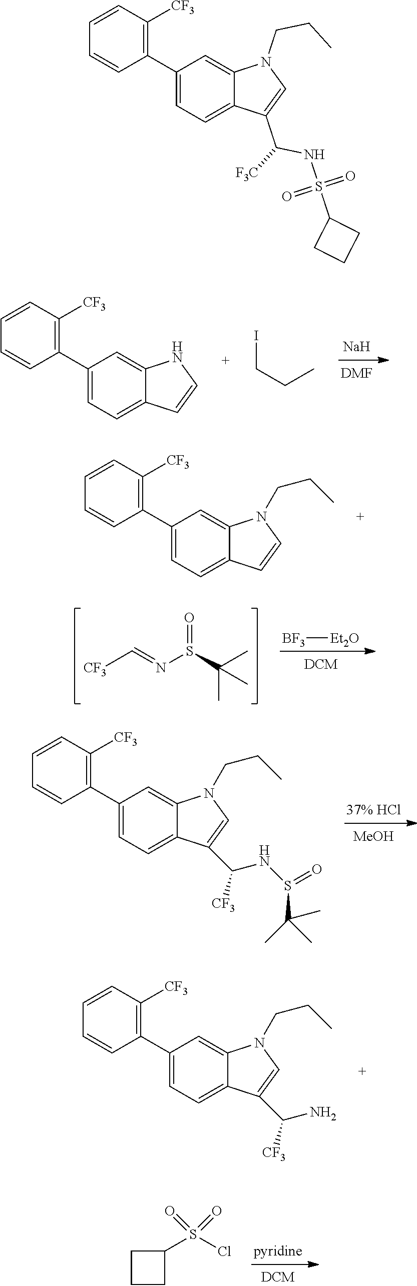

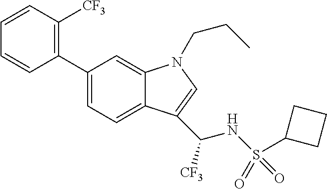 [SMILES: CCCn1cc([C@H](NS(=O)(=O)C2CCC2)C(F)(F)F)c2ccc(-c3ccccc3C(F)(F)F)cc21]

### Step 1: 1-propyl-6-(2-(trifluoromethyl)phenyl)-1H-indole

The title compound was prepared following the same general protocol as described for Step 1, Example 1, using 6-(2-(trifluoromethyl)phenyl)-1H-indole and 1-iodopropane. ESI-MS (m/z): 304.1 [M+1]⁺.

### Step 2: (S)-2-methyl-N—((S)-2,2,2-trifluoro-1-(1-propyl-6-(2-(trifluoromethyl)phenyl)-1H-indol-3-yl)ethyl)propane-2-sulfinamide

The title compound was prepared following the same general protocol as described for Step 3, Example 116, using 1-propyl-6-(2-(trifluoromethyl)phenyl)-1H-indole. ESI-MS (m/z): 505.2 [M+1]⁺.

### Step 3: (S)-2,2,2-trifluoro-1-(1-propyl-6-(2-(trifluoromethyl)phenyl)-1H-indol-3-yl)ethan-1-amine

The title compound was prepared following the same general protocol as described for Step 4, Example 116, using (S)-2-methyl-N—((S)-2,2,2-trifluoro-1-(1-propyl-6-(2-(trifluoromethyl)-phenyl)-1H-indol-3-yl)ethyl)propane-2-sulfinamide. ESI-MS (m/z): 384.1 [M-NH₂]⁺.

### Step 4: (S)—N-(2,2,2-trifluoro-1-(1-propyl-6-(2-(trifluoromethyl)phenyl)-1H-indol-3-yl)ethyl)cyclobutanesulfonamide

The title compound was prepared following the same general protocol as described for Step 5, Example 116, (S)-2,2,2-trifluoro-1-(1-propyl-6-(2-(trifluoromethyl)phenyl)-1H-indol-3-yl)ethan-1-amine and cyclobutanesulfonyl chloride. ESI-MS (m/z): 519.2 [M+1]⁺.

### Example 439: (S)-2-(3-(1-(cyclobutanesulfonamido)-2,2,2-trifluoroethyl)-1-cyclobutyl-1H-indol-6-yl)-3-fluorobenzamide (439)

### Step 1: (S)-2-(3-(1-amino-2,2,2-trifluoroethyl)-1-cyclobutyl-1H-indol-6-yl)-3-fluorobenzamide

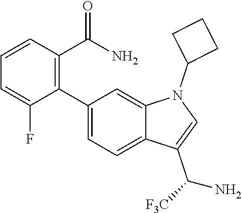 [SMILES: NC(=O)c1cccc(F)c1-c1ccc2c([C@H](N)C(F)(F)F)cn(C3CCC3)c2c1]

A mixture of (S)-1-(6-bromo-1-cyclobutyl-1H-indol-3-yl)-2,2,2-trifluoroethanamine (300 mg, 864.12 μmol), (2-cyano-6-fluoro-phenyl)boronic acid (156.77 mg, 950.53 μmol), Pd(dppf)Cl2 (126.46 mg, 172.82 μmol) and Cs2CO3 (703.87 mg, 2.16 mmol) in dioxane (4 mL) and water (1 mL) was stirred at 80° C. for 4 h under Nitrogen. The mixture was concentrated, the residue was purified by silica gel column (EA/PE=1:10 to MeOH/EA=1:5) to get (S)-2-(3-(1-amino-2,2,2-trifluoroethyl)-1-cyclobutyl-1H-indol-6-yl)-3-fluorobenzamide (290 mg, 715.36 μmol, 82.79% yield) as brown oil.

### Step 2: (S)-2-(3-(1-(cyclobutanesulfonamido)-2,2,2-trifluoroethyl)-1-cyclobutyl-1H-indol-6-yl)-3-fluorobenzamide

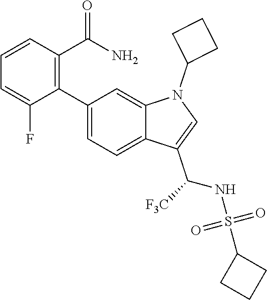 [SMILES: NC(=O)c1cccc(F)c1-c1ccc2c([C@H](NS(=O)(=O)C3CCC3)C(F)(F)F)cn(C3CCC3)c2c1]

A mixture of (S)-2-(3-(1-amino-2,2,2-trifluoroethyl)-1-cyclobutyl-1H-indol-6-yl)-3-fluorobenzamide (200 mg, 493.35 μmol) and cyclobutanesulfonyl chloride (152.56 mg, 986.71 μmol) in anhydrous pyridine (0.4 mL) was stirred at 100° C. for 4 hours in the microwave. The mixture was added CH2Cl2 and concentrated to get black oil, which was purified by silica gel column (MeOH/EA=1:5) to get black oil. which was purified by prep-HPLC to get (S)-2-(3-(1-(cyclobutanesulfonamido)-2,2,2-trifluoroethyl)-1-cyclobutyl-1H-indol-6-yl)-3-fluorobenzamide (22.41 mg, 42.80 μmol, 8.68% yield) as a white solid. ¹H NMR (400 MHz, CDCl₃) δ 7.79 (d, J=8.3 Hz, 1H), 7.66 (d, J=7.7 Hz, 1H), 7.47-7.37 (m, 3H), 7.30-7.26 (m, 1H), 7.22 (dd, J=8.3, 1.3 Hz, 1H), 5.53 (s, 1H), 5.39 (dd, J=15.6, 7.8 Hz, 1H), 5.19 (s, 1H), 5.03 (d, J=8.2 Hz, 1H), 4.86-4.76 (m, 1H), 3.87 (p, J=8.3 Hz, 1H), 2.63-2.34 (m, 6H), 2.27 (ddd, J=14.7, 7.3, 3.2 Hz, 2H), 2.03-1.85 (m, 4H). LCMS: 541.2 m/z [M+18]⁺.

### Example 440: (S)—N-(1-(6-(2-cyanophenyl)-5-fluoro-1-neopentyl-1H-indol-3-yl)-2,2,2-trifluoroethyl)cyclobutanesulfonamide (440)

### Step 1: (S)-2,2,2-trifluoro-1-(5-fluoro-1-neopentyl-6-(4,4,5,5-tetramethyl-1,3,2-dioxaborolan-2-yl)-1H-indol-3-yl)ethanamine

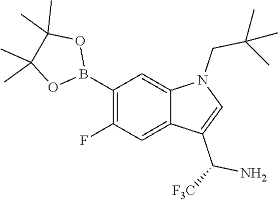 [SMILES: CC(C)(C)Cn1cc([C@H](N)C(F)(F)F)c2cc(F)c(B3OC(C)(C)C(C)(C)O3)cc21]

A mixture of (S)-1-(6-bromo-5-fluoro-1-neopentyl-1H-indol-3-yl)-2,2,2-trifluoroethanamine (300 mg, 786.98 mol), 4,4,5,5-tetramethyl-2-(4,4,5,5-tetramethyl-1,3,2-dioxaborolan-2-yl)-1,3,2-dioxaborolane (299.76 mg, 1.18 mmol), Pd(dppf)Cl₂ (57.58 mg, 78.70 μmol) and potassium acetate (231.71 mg, 2.36 mmol, 147.58 μL) in anhydrous dioxane (6 mL) was stirred at 110° C. for 30 min in the microwave under Nitrogen. The mixture was concentrated, the residue was purified by silica gel column (EA/PE=1:10 to 1:3) to get (S)-2,2,2-trifluoro-1-(5-fluoro-1-neopentyl-6-(4,4,5,5-tetramethyl-1,3,2-dioxaborolan-2-yl)-1H-indol-3-yl)ethanamine (336 mg, 784.55 μmol, 99.69% yield) as yellow oil.

### Step 2: (S)-2-(3-(1-amino-2,2,2-trifluoroethyl)-5-fluoro-1-neopentyl-1H-indol-6-yl)benzonitrile

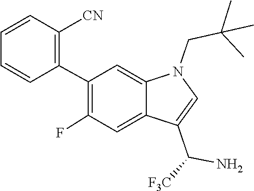 [SMILES: CC(C)(C)Cn1cc([C@H](N)C(F)(F)F)c2cc(F)c(-c3ccccc3C#N)cc21]

A mixture of (S)-2,2,2-trifluoro-1-(5-fluoro-1-neopentyl-6-(4,4,5,5-tetramethyl-1,3,2-dioxaborolan-2-yl)-1H-indol-3-yl)ethanamine (296 mg, 691.15 μmol), 2-bromobenzonitrile (188.70 mg, 1.04 mmol), Pd(dppf)Cl₂ (50.57 mg, 69.12 μmol) and K₂CO₃ (191.04 mg, 1.38 mmol) in dioxane (6 mL) and water (0.4 mL) was stirred at 80° C. for 3 h under Nitrogen. The mixture was concentrated, the residue was purified by silica gel column (EA/PE=1:10 to 1:1) to get (S)-2-(3-(1-amino-2,2,2-trifluoroethyl)-5-fluoro-1-neopentyl-1H-indol-6-yl)benzonitrile (270 mg, 669.29 μmol, 96.84% yield) as brown solid.

### Step 3: (S)—N-(1-(6-(2-cyanophenyl)-5-fluoro-1-neopentyl-1H-indol-3-yl)-2,2,2-trifluoroethyl)cyclobutanesulfonamide

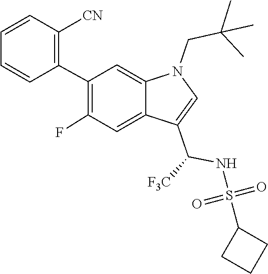 [SMILES: CC(C)(C)Cn1cc([C@H](NS(=O)(=O)C2CCC2)C(F)(F)F)c2cc(F)c(-c3ccccc3C#N)cc21]

A mixture of (S)-2-(3-(1-amino-2,2,2-trifluoroethyl)-5-fluoro-1-neopentyl-1H-indol-6-yl)benzonitrile (236 mg, 585.01 μmol) and cyclobutanesulfonyl chloride (245.51 mg, 1.11 mmol) in anhydrous pyridine (0.5 mL) was stirred at 100° C. for 4 hours in the microwave. To the mixture was added CH2Cl2 and concentrated to get black oil, which was purified by silica gel column (MeOH/EA=1:5) to get brown oil. which was purified by prep-HPLC to get (S)—N-(1-(6-(2-cyanophenyl)-5-fluoro-1-neopentyl-1H-indol-3-yl)-2,2,2-trifluoroethyl)cyclobutanesulfonamide (66.87 mg, 128.21 μmol, 21.92% yield) as a white solid. ¹H NMR (400 MHz, CDCl₃) δ 7.78 (dd, J=7.8, 0.9 Hz, 1H), 7.65 (td, J=7.7, 1.3 Hz, 1H), 7.56-7.50 (m, 2H), 7.47 (td, J=7.7, 1.2 Hz, 1H), 7.37 (d, J=6.0 Hz, 1H), 7.28 (s, 1H), 5.32 (dd, J=15.8, 7.9 Hz, 1H), 5.02 (d, J=8.4 Hz, 1H), 3.85 (d, J=4.0 Hz, 2H), 3.80 (dd, J=11.3, 5.3 Hz, 1H), 2.52-2.43 (m, 2H), 2.25 (ddd, J=15.0, 7.6, 5.2 Hz, 2H), 1.99-1.92 (m, 2H), 1.00 (s, 9H). LCMS: 539.2 m/z [M+18]⁺.

### Example 441: (S)—N-(1-(6-(4-cyanopyridin-3-yl)-5-fluoro-1-neopentyl-1H-indol-3-yl)-2,2,2-trifluoroethyl)cyclobutanesulfonamide (441)

### Step 1: (S)-3-(3-(1-amino-2,2,2-trifluoroethyl)-5-fluoro-1-neopentyl-1H-indol-6-yl)isonicotinonitrile

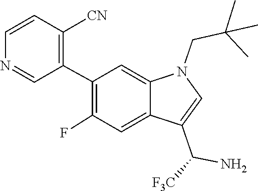 [SMILES: CC(C)(C)Cn1cc([C@H](N)C(F)(F)F)c2cc(F)c(-c3cnccc3C#N)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (449 mg, 838.72 μmol) and 3-bromopyridine-4-carbonitrile (184.19 mg, 1.01 mmol) in dioxane (10 mL) and H2O (2 mL) was added Pd(dppf)Cl₂ (61.39 mg, 83.87 μmol) and K₂CO₃ (231.49 mg, 1.68 mmol). The mixture was stirred at 90° C. for 5 hours. The mixture was quenched by H2O (10 mL). Then the mixture was extracted with DCM (20 mL*3) and the organic layers were washed by brine (10 ml). The product was purified by chromatograph silica on gel (EA-PE 0%˜50%, UV=254 nm). 3-[1-(2,2-dimethylpropyl)-5-fluoro-3-[rac-(1S)-1-amino-2,2,2-trifluoro-ethyl]indol-6-yl]pyridine-4-carbonitrile (180 mg, 392 μmol, 47% yield, 88% purity) as a yellow gum was obtained.

### Step 2: (S)—N-(1-(6-(4-cyanopyridin-3-yl)-5-fluoro-1-neopentyl-1H-indol-3-yl)-2,2,2-trifluoroethyl)cyclobutanesulfonamide

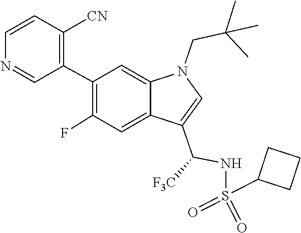 [SMILES: CC(C)(C)Cn1cc([C@H](NS(=O)(=O)C2CCC2)C(F)(F)F)c2cc(F)c(-c3cnccc3C#N)cc21]

A mixture of 3-[1-(2,2-dimethylpropyl)-5-fluoro-3-[rac-(1S)-1-amino-2,2,2-trifluoro-ethyl]indol-6-yl]pyridine-4-carbonitrile (180 mg, 356.08 μmol) and cyclobutanesulfonyl chloride (82.58 mg, 534.12 μmol) in Pyridine (2 mL) was stirred at 100° C. for 4 hours under microwave. The mixture was concentrated in vacuo and the product was purified by chromatograph silica on gel (EA:PE, 0%˜60%, UV=254 nm). A crude product (120 mg) was obtained. The product was purified by Prep HPLC (base). N-[rac-(1S)-1-[6-(4-cyano-3-pyridyl)-1-(2,2-dimethylpropyl)-5-fluoro-indol-3-yl]-2,2,2-trifluoro-ethyl]cyclobutanesulfonamide (20 mg, 38.27 μmol, 11% yield) as a white solid was obtained. 1H NMR (400 MHz, CDCl3) δ 8.77 (d, J=0.7 Hz, 1H), 8.70 (dd, J=5.0, 2.0 Hz, 1H), 7.57 (dd, J=5.1, 0.7 Hz, 1H), 7.52 (d, J=10.5 Hz, 1H), 7.30 (d, J=5.9 Hz, 1H), 7.25 (s, 1H), 5.28 (s, 1H), 5.11 (s, 1H), 3.83-3.75 (m, 3H), 2.42 (ddd, J=12.2, 11.1, 3.4 Hz, 2H), 2.23-2.14 (m, 2H), 1.94-1.86 (m, 2H), 0.92 (s, 9H). MS Found: 522.7[M+H]⁺.

### Example 442: (S)—N-(1-(1-(2,2-difluoropropyl)-6-(2-(trifluoromethyl)phenyl)-1H-indol-3-yl)-2,2,2-trifluoroethyl)cyclobutanesulfonamide (442)

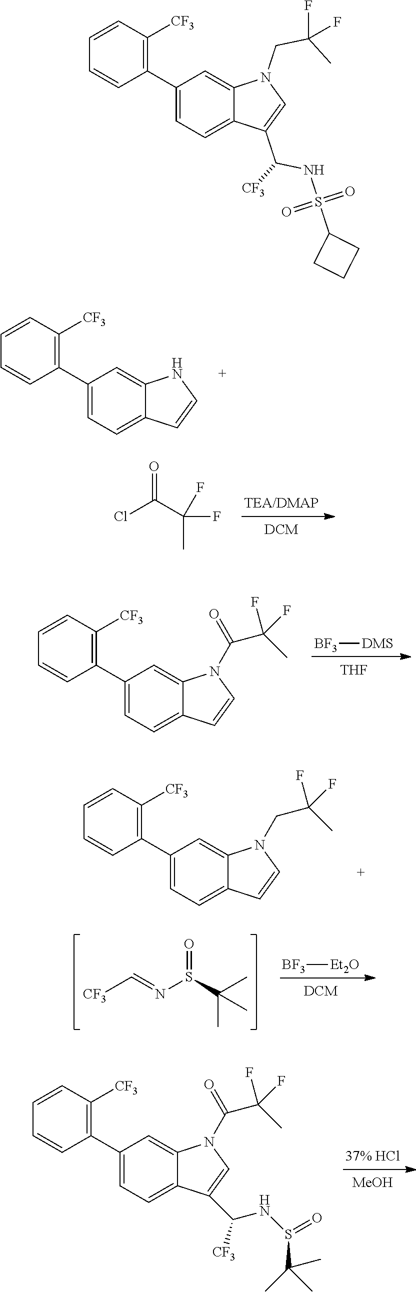

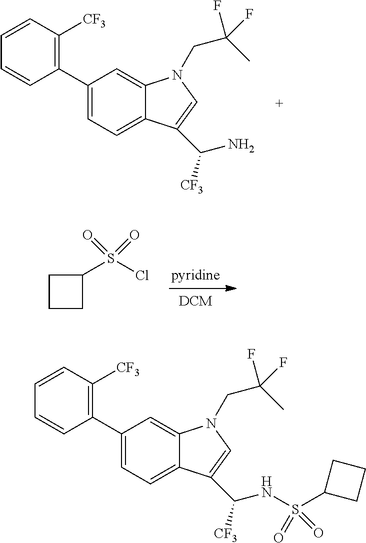 [SMILES: CC(F)(F)Cn1cc([C@H](N)C(F)(F)F)c2ccc(-c3ccccc3C(F)(F)F)cc21 ; O=S(=O)(Cl)C1CCC1 ; CC(F)(F)Cn1cc([C@H](NS(=O)(=O)C2CCC2)C(F)(F)F)c2ccc(-c3ccccc3C(F)(F)F)cc21]

### Step 1: 2,2-difluoro-1-(6-(2-(trifluoromethyl)phenyl)-1H-indol-1-yl)propan-1-one

The title compound was prepared following the same general protocol as described for Example 34, using 6-(2-(trifluoromethyl)phenyl)-1H-indole and 2,2-difluoropropanoyl chloride. ESI-MS (m/z): 354.1 [M+1]⁺.

### Step 2: 1-propyl-6-(2-(trifluoromethyl)phenyl)-1H-indole

The title compound was prepared following the same general protocol as described for Step 3, Example 1, using 2,2-difluoro-1-(6-(2-(trifluoromethyl)phenyl)-1H-indol-1-yl)propan-1-one. ESI-MS (m/z): 340.1 [M+1]⁺.

### Step 3: (S)-2-methyl-N—((S)-2,2,2-trifluoro-1-(1-propyl-6-(2-(trifluoromethyl)phenyl)-1H-indol-3-yl)ethyl)propane-2-sulfinamide

The title compound was prepared following the same general protocol as described for Step 3, Example 116, using 1-propyl-6-(2-(trifluoromethyl)phenyl)-1H-indole. ESI-MS (m/z): 541.2 [M+1]⁺.

### Step 4: (S)-2,2,2-trifluoro-1-(1-propyl-6-(2-(trifluoromethyl)phenyl)-1H-indol-3-yl)ethan-1-amine

The title compound was prepared following the same general protocol as described for Step 4, Example 116, using (S)-2-methyl-N—((S)-2,2,2-trifluoro-1-(1-propyl-6-(2-(trifluoromethyl)-phenyl)-1H-indol-3-yl)ethyl)propane-2-sulfinamide. ESI-MS (m/z): 420.1 [M-NH₂]⁺.

### Step 5: (S)—N-(2,2,2-trifluoro-1-(1-propyl-6-(2-(trifluoromethyl)phenyl)-1H-indol-3-yl)ethyl)cyclobutanesulfonamide

The title compound was prepared following the same general protocol as described for Step 5, Example 116, (S)-2,2,2-trifluoro-1-(1-propyl-6-(2-(trifluoromethyl)phenyl)-1H-indol-3-yl)ethan-1-amine and cyclobutanesulfonyl chloride. ESI-MS (m/z): 555.2 [M+1]⁺.

### Example 444: (S)—N-(2,2,2-trifluoro-1-(5-fluoro-1-neopentyl-6-(3-(trifluoromethyl)pyridin-2-yl)-1H-indol-3-yl)ethyl)cyclobutanesulfonamide (444)

### Step 1: (S)-2,2,2-trifluoro-1-(5-fluoro-1-neopentyl-6-(3-(trifluoromethyl)pyridin-2-yl)-1H-indol-3-yl)ethan-1-amine

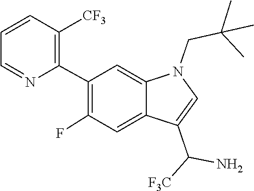 [SMILES: CC(C)(C)Cn1cc(C(N)C(F)(F)F)c2cc(F)c(-c3ncccc3C(F)(F)F)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 3-bromo-2-(trifluoromethyl)pyridine (172.87 mg, 764.94 μmol) in dioxane (10 mL) and H2O (2 mL) was added Pd(dppf)Cl2 (43.07 mg, 58.84 μmol) and K2CO3 (162.40 mg, 1.18 mmol). The mixture was purged by N2 for 10 seconds. The mixture was stirred at 85° C. for 5 hours. The mixture was combined with crude material from a previous test reaction using the same condition. The product was purified by chromatograph silica on gel (EA:PE, 0%˜60%, UV=254 nm). rac-(1S)-1-[1-(2,2-Dimethylpropyl)-5-fluoro-6-[3-(trifluoromethyl)-2-pyridyl]indol-3-yl]-2,2,2-trifluoro-ethanamine (160 mg, 358 μmol, 61% yield) as a yellow gum was obtained.

### Step 2: (S)—N-(2,2,2-trifluoro-1-(5-fluoro-1-neopentyl-6-(3-(trifluoromethyl)pyridin-2-yl)-1H-indol-3-yl)ethyl)cyclobutanesulfonamide

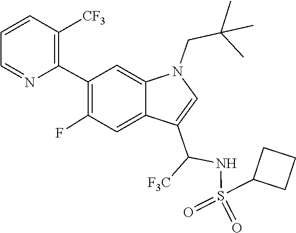 [SMILES: CC(C)(C)Cn1cc(C(NS(=O)(=O)C2CCC2)C(F)(F)F)c2cc(F)c(-c3ncccc3C(F)(F)F)cc21]

A mixture of rac-(1S)-1-[1-(2,2-dimethylpropyl)-5-fluoro-6-[3-(trifluoromethyl)-2-pyridyl]indol-3-yl]-2,2,2-trifluoro-ethanamine (160 mg, 357.63 μmol) in Pyridine (2 mL) was added cyclobutanesulfonyl chloride (160 mg, 1.03 mmol). The mixture was purged by N2 for 20 seconds. The mixture was stirred at 100° C. for 4 hours under microwave and N2. The mixture was concentrated in vacuo and the product was purified by Prep HPLC. N-[rac-(1S)-1-[1-(2,2-dimethylpropyl)-5-fluoro-6-[3-(trifluoromethyl)-2-pyridyl]indol-3-yl]-2,2,2-trifluoro-ethyl]cyclobutanesulfonamide (16 mg, 28.29 μmol, 7.91% yield) as a light yellow solid was obtained. 1H NMR (400 MHz, CDCl3) δ 8.85 (d, J=4.6 Hz, 1H), 8.10 (dd, J=8.0, 1.3 Hz, 1H), 7.47 (dd, J=11.6, 5.6 Hz, 2H), 7.30 (d, J=5.8 Hz, 1H), 7.26 (s, 1H), 5.31 (dd, J=15.6, 7.8 Hz, 1H), 5.09 (d, J=7.8 Hz, 1H), 3.78 (dd, J=16.3, 7.9 Hz, 3H), 2.46 (dd, J=19.9, 11.4 Hz, 2H), 2.25-2.11 (m, 2H), 1.93 (td, J=9.3, 6.8 Hz, 2H), 0.96 (s, 9H). MS Found: 566.2[M+H]⁺.

### Example 445: N-(1-(1-neopentyl-6-(2-(trifluoromethyl)phenyl)-1H-indol-3-yl)ethyl-1-d)cyclobutanesulfonamide (445)

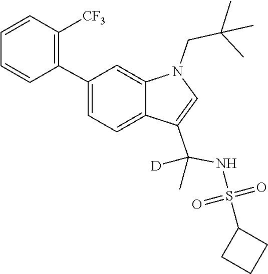 [SMILES: [2H]C(C)(NS(=O)(=O)C1CCC1)c1cn(CC(C)(C)C)c2cc(-c3ccccc3C(F)(F)F)ccc12]

### Step 1: N-(1-(1-neopentyl-6-(2-(trifluoromethyl)phenyl)-1H-indol-3-yl)ethylidene)cyclobutanesulfonamide

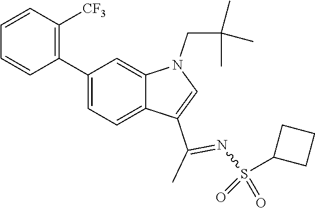 [SMILES: CC(=NS(=O)(=O)C1CCC1)c1cn(CC(C)(C)C)c2cc(-c3ccccc3C(F)(F)F)ccc12]

The title compound was prepared following the same general protocol as described for Step 4, Example 113, using 1-(1-neopentyl-6-(2-(trifluoromethyl)phenyl)-1H-indol-3-yl)ethan-1-one and cyclobutanesulfonamide. ESI-MS (m/z): 490.78 [M+1]⁺.

### Step 2: N-(1-(1-neopentyl-6-(2-(trifluoromethyl)phenyl)-1H-indol-3-yl)ethyl-1-d)cyclobutanesulfonamide

To a solution of N-(1-(1-neopentyl-6-(2-(trifluoromethyl)phenyl)-1H-indol-3-yl)ethylidene)cyclobutanesulfonamide (0.06 g, 0.122 mmol) in THF (1 mL) and 1 drop of D₂O at rt was added NaBD₄ (0.011 g, 0.244 mmol), The reaction was monitored by anal. HPLC for completion. When complete, the reaction was quenched with water and the crude mixture was purified by prep HPLC to obtain the title compound. ¹HNMR (CDCl₃, 400 MHz) δ 7.95 (d, J=8.0 Hz, 1H), 7.91 (d, J=8.0 Hz, 1H), 7.75 (t, J=8.0 Hz, 1H), 7.67 (t, J=8.0 Hz, 1H), 7.58 (d, J=8.0 Hz, 1H), 7.49 (s, 1H), 7.26 (d, J=8.0 Hz, 2H), 4.82 (s, 1H), 4.09 (s, 2H), 3.69-3.61 (m, 1H), 2.67-2.49 (m, 2H), 2.32-2.29 (m, 1H), 2.27 (s, 1H), 2.19-1.98 (m, 3H), 1.5 (s, 2H), 1.10 (s, 9H)

### Example 446: N-((1S)-1-(1-(sec-butyl)-6-(2-(trifluoromethyl)phenyl)-1H-indol-3-yl)-2,2,2-trifluoroethyl)cyclobutanesulfonamide (446)

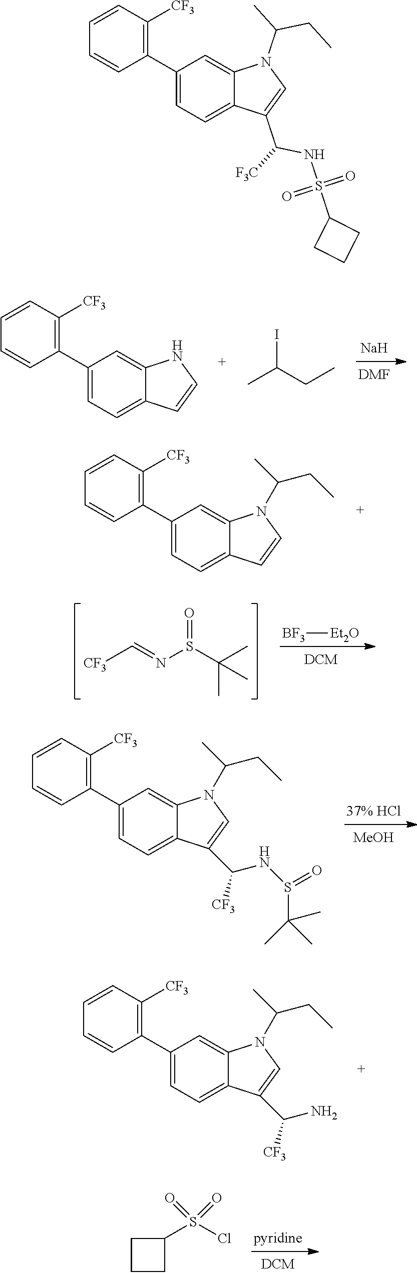

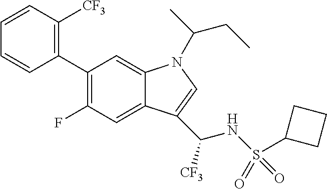 [SMILES: CCC(C)n1cc([C@H](NS(=O)(=O)C2CCC2)C(F)(F)F)c2cc(F)c(-c3ccccc3C(F)(F)F)cc21]

### Step 1: 1-(sec-butyl)-6-(2-(trifluoromethyl)phenyl)-1H-indole

The title compound was prepared following the same general protocol as described for Step 1, Example 1, using 6-(2-(trifluoromethyl)phenyl)-1H-indole and 2-iodobutane. ESI-MS (m/z): 318.1 [M+1]⁺.

### Step 2: (S)—N-((1S)-1-(1-(sec-butyl)-6-(2-(trifluoromethyl)phenyl)-1H-indol-3-yl)-2,2,2-trifluoroethyl)-2-methylpropane-2-sulfinamide

The title compound was prepared following the same general protocol as described for Step 3, Example 116, using 1-(sec-butyl)-6-(2-(trifluoromethyl)phenyl)-1H-indole. ESI-MS (m/z): 519.2 [M+1]⁺.

### Step 3: (1S)-1-(1-(sec-butyl)-6-(2-(trifluoromethyl)phenyl)-1H-indol-3-yl)-2,2,2-trifluoroethan-1-amine

The title compound was prepared following the same general protocol as described for Step 4, Example 116, using (S)—N-((1S)-1-(1-(sec-butyl)-6-(2-(trifluoromethyl)phenyl)-1H-indol-3-yl)-2,2,2-trifluoroethyl)-2-methylpropane-2-sulfinamide. ESI-MS (m/z): 398.1 [M-NH₂]⁺.

### Step 4: N-((1S)-1-(1-(sec-butyl)-6-(2-(trifluoromethyl)phenyl)-1H-indol-3-yl)-2,2,2-trifluoroethyl)cyclobutanesulfonamide

The title compound was prepared following the same general protocol as described for Step 5, Example 116, (1S)-1-(1-(sec-butyl)-6-(2-(trifluoromethyl)phenyl)-1H-indol-3-yl)-2,2,2-trifluoroethan-1-amine and cyclobutanesulfonyl chloride. ESI-MS (m/z): 533.2 [M+1]⁺.

### Example 454: N-[(1S)-2,2,2-trifluoro-1-[5-fluoro-1-isobutyl-6-(5,6,7,8-tetrahydroquinolin-4-yl)indol-3-yl]ethyl]cyclopropanesulfonamide (454)

### Step 1: 6-bromo-5-fluoro-1-isobutyl-1H-indole

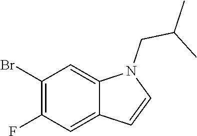

6-bromo-5-fluoro-1H-indole (22 g, 102.79 mmol) in DMF (4 mL) was added to a suspension of NaH (8.63 g, 359.76 mmol) in anhydrous DMF (150 mL) in ice-water under nitrogen. The mixture was stirred for 1 h at room temperature, at which time the reaction was cooled in an ice bath followed by the addition of 1-iodo-2-methyl-propane (24.59 g, 133.62 mmol, 15.37 mL) and the resulting mixture was stirred at 40° C. overnight under nitrogen. After quenching with water the reaction mixture was partitioned between ethyl acetate (50 mL) and water (50 mL). The aqueous layer was extracted using ethyl acetate (2×50 mL) and the combined organic layers were washed sequentially with brine (3×50 mL) and dried over anhydrous sodium sulfate. After the solvent was removed in vacuo, flash chromatography (silica gel, 100% Petroleum ether) yielded 6-bromo-5-fluoro-1-isobutyl-indole (9.7 g, 27.65 mmol, 26.90% yield, 77% purity) as pale yellow oil.

### Step 2: (S)—N—((S)-1-(6-bromo-5-fluoro-1-isobutyl-1H-indol-3-yl)-2,2,2-trifluoroethyl)-2-methylpropane-2-sulfinamide

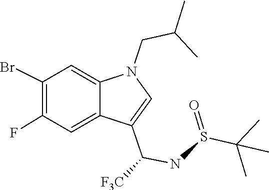

The mixture of 6-bromo-5-fluoro-1-isobutyl-indole (8.7 g, 32.21 mmol) in anhydrous CH₂Cl₂ (35 mL) was cooled to −10° C., BF₃·OEt₂ (48.32 mol, 6 mL) was added slowly, followed by addition of a solution of (S,E)-2-methyl-N-(2,2,2-trifluoroethylidene)propane-2-sulfinamide in DCM. The mixture was then continued to stir for 1-2 h. The mixture was diluted with DCM, followed by water. The aqueous layer was extracted with DCM twice. Organic layers were combined and washed with brine and concentrated in vacuo to obtain the crude (S)—N-[(1S)-1-(6-bromo-5-fluoro-1-isobutyl-indol-3-yl)-2,2,2-trifluoro-ethyl]-2-methyl-propane-2-sulfinamide.

### Step 3: (S)-1-(6-bromo-5-fluoro-1-isobutyl-1H-indol-3-yl)-2,2,2-trifluoroethanamine

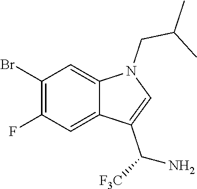 [SMILES: CC(C)Cn1cc([C@H](N)C(F)(F)F)c2cc(F)c(Br)cc21]

A mixture of(S)—N-[(1S)-1-(6-bromo-5-fluoro-1-isobutyl-indol-3-yl)-2,2,2-trifluoro-ethyl]-2-methyl-propane-2-sulfinamide (10.1 g, 21.43 mmol) in 4M HCl(g)/MeOH (50 mL) was stirred stirred at RT for 2 h. the mixture was concentrated. The purity of the residue was 72% by HPLC after two purification with METE, the residue was basified to pH=9 with sat. NaHCO3. extracted with EtOAc (2*20 mL), the combined organic phases were washed with brine, dried with anhydrous Na2SO4 and filtered. The filtrate was concentrated to give (1S)-1-(6-bromo-5-fluoro-1-isobutyl-indol-3-yl)-2,2,2-trifluoro-ethanamine (8.2 g, 22.33 mmol). This (1 g) was purified by prep-HPLC to get pure product (650 mg).

### Step 4: (S)—N-(1-(6-bromo-5-fluoro-1-isobutyl-1H-indol-3-yl)-2,2,2-trifluoroethyl)cyclopropanesulfonamide

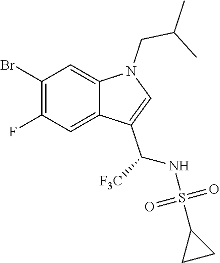 [SMILES: CC(C)Cn1cc([C@H](NS(=O)(=O)C2CC2)C(F)(F)F)c2cc(F)c(Br)cc21]

To a stirred solution of (1S)-1-(6-bromo-5-fluoro-1-isobutyl-indol-3-yl)-2,2,2-trifluoro-ethanamine (2.2 g, 5.99 mmol) in pyridine (15 mL) was added cyclopropanesulfonyl chloride (2.53 g, 17.97 mmol, 1.83 mL) at RT. After the addition, the mixture was stirred at RT for 7 h under Nitrogen. The mixture was concentrated at 50° C. in vacuo. The residue was dissolved with MeOH (3 mL) and purified by prep-HPLC to get N-[(1S)-1-(6-bromo-5-fluoro-1-isobutyl-indol-3-yl)-2,2,2-trifluoro-ethyl]cyclopropanesulfonamide (1.13 g, 2.40 mmol, 40.02% yield) as a white solid. ¹H NMR (400 MHz, DMSO) δ 8.59 (d, J=9.4 Hz, 1H), 7.96 (d, J=5.9 Hz, 1H), 7.87 (d, J=10.1 Hz, 1H), 7.74 (s, 1H), 5.59-5.32 (m, 1H), 4.01 (d, J=7.3 Hz, 2H), 2.35-2.21 (m, 1H), 2.13-1.98 (m, 1H), 0.95-0.77 (m, 8H), 0.74-0.57 (m, 2H). LCMS: 470.9 m/z [M+H]⁺.

### Step 5: (S)—N-(2,2,2-trifluoro-1-(5-fluoro-1-isobutyl-6-(4,4,5,5-tetramethyl-1,3,2-dioxaborolan-2-yl)-1H-indol-3-yl)ethyl)cyclopropanesulfonamide

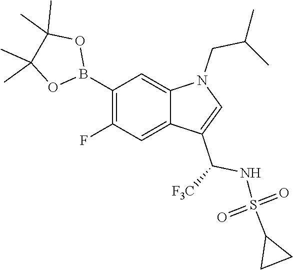 [SMILES: CC(C)Cn1cc([C@H](NS(=O)(=O)C2CC2)C(F)(F)F)c2cc(F)c(B3OC(C)(C)C(C)(C)O3)cc21]

A mixture of N-[(1S)-1-(6-bromo-5-fluoro-1-isobutyl-indol-3-yl)-2,2,2-trifluoro-ethyl]cyclopropanesulfonamide (490 mg, 1.04 mmol), 4,4,5,5-tetramethyl-2-(4,4,5,5-tetramethyl-1,3,2-dioxaborolan-2-yl)-1,3,2-dioxaborolane (528.02 mg, 2.08 mmol) and KOAc (306.10 mg, 3.12 mmol) in anhydrous dioxane (10 mL) was stirred at 80° C. overnight under Nitrogen. The mixture was concentrated, the residue was purified by silica gel column (EA/PE=0-20%) to get N-[(1S)-2,2,2-trifluoro-1-[5-fluoro-1-isobutyl-6-(4,4,5,5-tetramethyl-1,3,2-dioxaborolan-2-yl)indol-3-yl]ethyl]cyclopropanesulfonamide (750 mg, 1.45 mmol) as light yellow gum.

### Step 6: N-[(1S)-2,2,2-trifluoro-1-[5-fluoro-1-isobutyl-6-(5,6,7,8-tetrahydroquinolin-4-yl)indol-3-yl]ethyl]cyclopropanesulfonamide

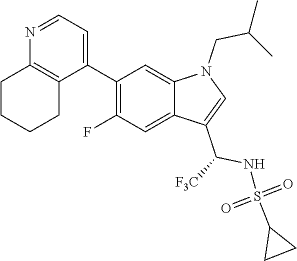 [SMILES: CC(C)Cn1cc([C@H](NS(=O)(=O)C2CC2)C(F)(F)F)c2cc(F)c(-c3ccnc4c3CCCC4)cc21]

5,6,7,8-tetrahydroquinolin-4-yl trifluoromethanesulfonate (200 mg, 711.11 μmol), N-[(1S)-2,2,2-trifluoro-1-[5-fluoro-1-isobutyl-6-(4,4,5,5-tetramethyl-1,3,2-dioxaborolan-2-yl)indol-3-yl]ethyl]cyclopropanesulfonamide (368.62 mg, 711.11 μmol), dipotassium;carbonate (196.56 mg, 1.42 mmol, 85.83 uL) and palladium;tetrakistriphenylphosphane (164.35 mg, 142.22 μmol) were added to the sealed tube (20 ml). Then dioxane (2.5 mL) and H₂O (0.5 mL) were added to the mixture. The mixture was stirred at 100° C. for 2 h. Water (5 mL) was added to the mixture. The mixture was extracted with EA (50 ml). The organic layer was dried over anhydrous Na₂SO₄, filtered and concentrated. The residue obtained was purified by prep-HPLC to give N-[(1S)-2,2,2-trifluoro-1-[5-fluoro-1-isobutyl-6-(5,6,7,8-tetrahydroquinolin-4-yl)indol-3-yl]ethyl]cyclopropanesulfonamide (20 mg, 37.70 μmol, 5.30% yield, 98.7% purity) as a white solid. 1H NMR (400 MHz, MeOD) δ 8.33 (d, J=5.0 Hz, 1H), 7.63-7.56 (m, 2H), 7.34 (d, J=5.9 Hz, 1H), 7.16 (d, J=5.0 Hz, 1H), 5.43 (q, J=7.9 Hz, 1H), 4.03 (d, J=7.4 Hz, 2H), 3.00 (t, J=6.5 Hz, 2H), 2.62 (s, 2H), 2.40-2.30 (m, 1H), 2.19 (dt, J=13.6, 6.9 Hz, 1H), 1.95 (dt, J=12.6, 6.5 Hz, 2H), 1.77 (s, 2H), 1.04 (tt, J=8.8, 4.3 Hz, 2H), 0.93 (dd, J=6.6, 3.6 Hz, 6H), 0.88-0.75 (m, 2H). MS Found: 523.8 [M+H]⁺.

### Example 455: (S)—N-(2,2,2-trifluoro-1-(5-fluoro-1-isobutyl-6-(pyrimidin-4-yl)-1H-indol-3-yl)ethyl)cyclopropanesulfonamide (455)

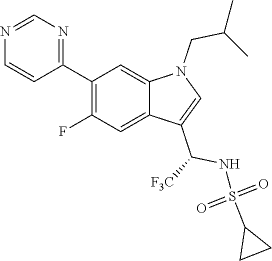 [SMILES: CC(C)Cn1cc([C@H](NS(=O)(=O)C2CC2)C(F)(F)F)c2cc(F)c(-c3ccncn3)cc21]

A mixture of N-[(1S)-2,2,2-trifluoro-1-[5-fluoro-1-isobutyl-6-(4,4,5,5-tetramethyl-1,3,2-dioxaborolan-2-yl)indol-3-yl]ethyl]cyclopropanesulfonamide (280.91 mg, 541.91 μmol), 4-bromopyrimidine;hydrobromide (100 mg, 416.85 μmol), Pd(PPh3)4 (192.68 mg, 166.74 μmol) and K2CO3 (345.53 mg, 2.50 mmol) in dioxane (3 mL) and water (0.5 mL) was stirred at 80° C. overnight under Nitrogen. The mixture was concentrated in vacuo to dryness, the residue was diluted with EtOAc, filtered through silica gel, the filtrate was concentrated to get brown oil, which was purified by prep-HPLC to get N-[(1S)-2,2,2-trifluoro-1-(5-fluoro-1-isobutyl-6-pyrimidin-4-yl-indol-3-yl)ethyl]cyclopropanesulfonamide (21.56 mg, 45.83 μmol) as a yellow solid. ¹H NMR (400 MHz, DMSO) δ 9.31 (s, 1H), 8.87 (s, 1H), 8.64 (s, 1H), 8.22 (d, J=4.4 Hz, 1H), 8.06-7.80 (m, 3H), 5.52 (d, J=6.6 Hz, 1H), 4.10 (d, J=5.7 Hz, 2H), 2.31 (s, 1H), 2.13 (s, 1H), 1.02-0.52 (m, 10H). LCMS: 470.8 m/z [M+H]⁺.

### Compound 456: (S)—N-(2,2,2-trifluoro-1-(5-fluoro-1-isobutyl-6-(4-methyl-1H-pyrazol-5-yl)-1H-indol-3-yl)ethyl)cyclopropanesulfonamide (456)

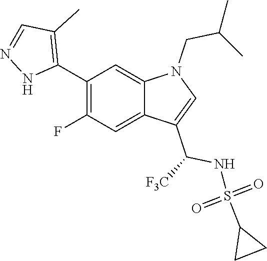 [SMILES: Cc1cn[nH]c1-c1cc2c(cc1F)c([C@H](NS(=O)(=O)C1CC1)C(F)(F)F)cn2CC(C)C]

A mixture of N-[(1S)-2,2,2-trifluoro-1-[5-fluoro-1-isobutyl-6-(4,4,5,5-tetramethyl-1,3,2-dioxaborolan-2-yl)indol-3-yl]ethyl]cyclopropanesulfonamide (180 mg, 347.24 μmol) in dioxane (3 mL) and water (0.7 mL) was stirred at 80° C. overnight under Nitrogen. The mixture was diluted with EtOAc, filtered through silica gel, the filtrate was concentrated to get brown oil, which was purified by prep-HPLC to get N-[(1S)-2,2,2-trifluoro-1-[5-fluoro-1-isobutyl-6-(4-methyl-1H-pyrazol-5-yl)indol-3-yl]ethyl]cyclopropanesulfonamide (7.0 mg, 14.81 μmol) as a white solid. ¹H NMR (400 MHz, CDCl3) δ 7.61-7.26 (m, 3H), 7.22 (s, 1H), 5.6-5.8 (br. s, 2H), 5.34 (d, J=6.8 Hz, 1H), 3.64 (dd, J=18.9, 6.4 Hz, 2H), 2.47 (s, 1H), 2.28-1.89 (m, 4H), 1.36-0.45 (m, 10H). LCMS: 472.8 m/z [M+H]⁺.

### Example 457: N-[(1S)-2,2,2-trifluoro-1-(5-fluoro-1-isobutyl-6-thiazol-4-yl-indol-3-yl)ethyl]cyclopropanesulfonamide (457)

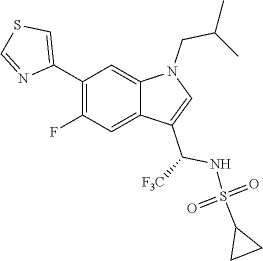 [SMILES: CC(C)Cn1cc([C@H](NS(=O)(=O)C2CC2)C(F)(F)F)c2cc(F)c(-c3cscn3)cc21]

A mixture of N-[(1S)-2,2,2-trifluoro-1-[5-fluoro-1-isobutyl-6-(4,4,5,5-tetramethyl-1,3,2-dioxaborolan-2-yl)indol-3-yl]ethyl]cyclopropanesulfonamide (109.99 mg, 212.18 μmol), 4-bromothiazole (104.41 mg, 636.54 μmol, 56.74 uL), Pd(PPh3)4 (98.07 mg, 84.87 μmol) and K2CO3 (87.84 mg, 636.54 μmol) in dioxane and water was stirred at 80° C. overnight under nitrogen. The mixture was diluted with EtOAc, filtered through diatomite, the filtrate was concentrated to get black oi, which was purified by prep-HPLC to get N-[(1S)-2,2,2-trifluoro-1-(5-fluoro-1-isobutyl-6-thiazol-4-yl-indol-3-yl)ethyl]cyclopropanesulfonamide (23.89 mg, 50.24 μmol, 23.68% yield). ¹H NMR (400 MHz, DMSO) δ 9.24 (s, 1H), 8.60 (s, 1H), 8.21 (d, J=5.5 Hz, 1H), 7.97 (s, 1H), 7.90-7.68 (m, 2H), 5.49 (s, 1H), 4.06 (d, J=6.4 Hz, 2H), 2.30 (s, 1H), 2.13 (d, J=5.9 Hz, 1H), 1.03-0.57 (m, 10H). LCMS: 475.8 m/z [M+H]⁺.

### Example 458: (S)—N-(2,2,2-trifluoro-1-(1-neopentyl-6-(pyridin-4-yl)-5-(trifluoromethyl)-1H-indol-3-yl)ethyl)cyclopropanesulfonamide (458)

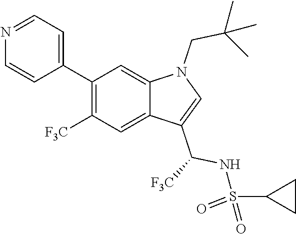 [SMILES: CC(C)(C)Cn1cc([C@H](NS(=O)(=O)C2CC2)C(F)(F)F)c2cc(C(F)(F)F)c(-c3ccncc3)cc21]

The title compound was prepared following the same general protocol as described for Step 5, Example 1, using (S)—N-(1-(6-bromo-1-neopentyl-5-(trifluoromethyl)-1H-indol-3-yl)-2,2,2-trifluoroethyl)cyclopropanesulfonamide and 4-(4,4,5,5-tetramethyl-1,3,2-dioxaborolan-2-yl)pyridine. HNMR (CDCl₃, 400 MHz) δ 8.64 (s, 2H), 8.18 (s, 1H), 7.36 (s, 1H), 7.31 (d, J=4.0 Hz, 2H), 7.22 (s, 1H), 5.79 (d, J=12.0 Hz, 1H), 5.47 (q, J=8.0 Hz, 1H), 3.89 (s, 2H), 2.51-2.45 (m, 1H), 1.28-1.24 (m, 1H), 1.17-1.12 (m, 1H), 1.03-0.91 (m, 11H)

### Example 459: (S)—N-(2,2,2-trifluoro-1-(5-fluoro-1-neopentyl-6-(2-(trifluoromethyl)phenyl)-1H-indol-3-yl)ethyl)oxetane-3-sulfonamide (459)

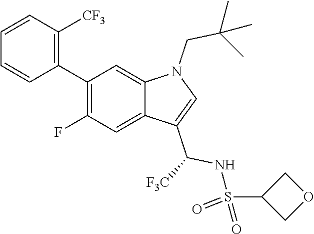 [SMILES: CC(C)(C)Cn1cc([C@H](NS(=O)(=O)C2COC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and oxetane-3-sulfonyl chloride. ¹HNMR (CDCl₃, 400 MHz) δ 7.79 (d, J=8.0 Hz, 1H), 7.59 (t, J=8.0 Hz, 1H), 7.53 (t, J=8.0 Hz, 1H), 7.44-7.34 (m, 2H), 7.26-7.23 (m, 2H), 5.36 (q, J=8.0 Hz, 1H), 5.24-5.18 (m, 1H), 4.95-4.71 (m, 4H), 4.51-4.91 (m, 1H), 3.91-3.81 (m, 2H), 0.97 (s, 9H)

### Example 460: (S)—N-(1-(6-(2-cyano-6-fluorophenyl)-1-neopentyl-1H-indol-3-yl)-2,2,2-trifluoroethyl)cyclopropanesulfonamide (460)

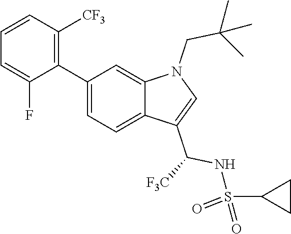 [SMILES: CC(C)(C)Cn1cc([C@H](NS(=O)(=O)C2CC2)C(F)(F)F)c2ccc(-c3c(F)cccc3C(F)(F)F)cc21]

The title compound was prepared following the same general protocol as described for Step 5, Example 1, using 2-bromo-3-fluorobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.83 (d, J=8.0 Hz, 1H), 7.59 (dd, J=8.0 Hz, 4.0 Hz, 1H), 7.49-7.38 (m, 3H), 7.28 (s, 2H), 5.44 (q, J=8.0 Hz, 1H), 5.16 (d, J=8.0 Hz, 1H), 3.88 (s, 2H), 2.45-2.39 (m, 1H), 1.43-1.21 (m, 2H), 1.18 (s, 9H), 1.16-1.00 (m, 2H)

### Example 461: N—((S)-2,2,2-trifluoro-1-(5-fluoro-1-neopentyl-6-(2-(trifluoromethyl)phenyl)-1H-indol-3-yl)ethyl)tetrahydrofuran-3-sulfonamide (461)

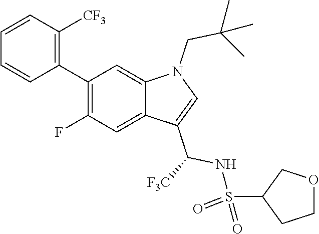 [SMILES: CC(C)(C)Cn1cc([C@H](NS(=O)(=O)C2CCOC2)C(F)(F)F)c2cc(F)c(-c3ccccc3C(F)(F)F)cc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-indol-3-yl)ethan-1-amine and tetrahydrofuran-3-sulfonyl chloride. ¹HNMR (CDCl₃, 400 MHz) δ 7.79 (d, J=8.0 Hz, 1H), 7.59 (t, J=8.0 Hz, 1H), 7.52 (t, J=8.0 Hz, 1H), 7.46-7.38 (m, 2H), 7.31-7.21 (m, 2H), 4.89-4.87 (m, 1H), 4.77 (q, J=8.0 Hz, 1H), 4.49-4.45 (m, 1H), 4.21-4.11 (m, 1H), 3.88 (t, J=8.0 Hz, 2H), 3.57 (s, 2H), 3.37-3.31 (m, 1H), 2.62 (d, J=12.0 Hz, 1H), 2.42 (d, J=12.0 Hz, 1H), 0.98 (s, 9H)

### Example 462: (S)—N-(1-(6-(2-cyanophenyl)-1-neopentyl-1H-indol-3-yl)-2,2,2-trifluoroethyl)cyclopropanesulfonamide (462)

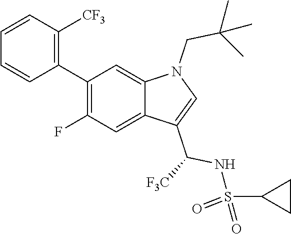 [SMILES: CC(C)(C)Cn1cc([C@H](NS(=O)(=O)C2CC2)C(F)(F)F)c2cc(F)c(-c3ccccc3C(F)(F)F)cc21]

The title compound was prepared following the same general protocol as described for Step 5, Example 1, using 2-bromobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.82 (d, J=8.0 Hz, 1H), 7.76 (d, J=8.0 Hz, 1H), 7.64 (t, J=8.0 Hz, 1H), 7.57 (s, 2H), 7.42 (t, J=8.0 Hz, 1H), 7.31 (d, J=8.0 Hz, 1H), 7.28 (s, 1H), 5.44 (q, J=8.0 Hz, 1H), 5.32 (d, J=8.0 Hz, 1H), 3.89 (s, 2H), 2.44-2.38 (m, 1H), 1.43-1.21 (m, 2H), 0.98 (s, 9H), 0.95-0.89 (m, 2H)

### Example 463: (S)—N-(2,2,2-trifluoro-1-(6-(2-methyl-6-(trifluoromethyl)phenyl)-1-neopentyl-1H-indol-3-yl)ethyl)cyclopropanesulfonamide (463)

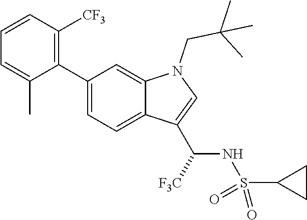 [SMILES: Cc1cccc(C(F)(F)F)c1-c1ccc2c([C@H](NS(=O)(=O)C3CC3)C(F)(F)F)cn(CC(C)(C)C)c2c1]

The title compound was prepared following the same general protocol as described for Step 5, Example 1, using 2-bromo-1-methyl-3-(trifluoromethyl)benzene and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. ¹HNMR (CDCl₃, 400 MHz) δ 7.74 (t, J=8.0 Hz, 1H), 7.62 (d, J=8.0 Hz, 1H), 7.45 (d, J=4.0 Hz, 1H), 7.37 (t, J=8.0 Hz, 1H), 7.18 (s, 1H), 7.14 (s, 1H), 6.96 (d, J=8.0 Hz, 1H), 5.49-5.39 (m, 1H), 5.22-5.10 (m, 1H), 3.93-3.82 (m, 2H), 2.46-2.33 (m, 1H), 2.09 (d, J=4.0 Hz, 3H), 1.19-1.10 (m, 2H), 0.96 (s, 9H), 0.91-0.84 (m, 2H)

### Example 464: (S)—N-(1-(6-(2-chloro-6-fluorophenyl)-1-neopentyl-1H-indol-3-yl)-2,2,2-trifluoroethyl)cyclopropanesulfonamide (464)

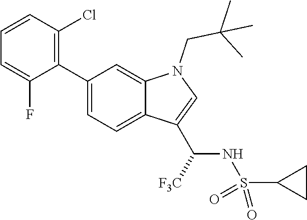 [SMILES: CC(C)(C)Cn1cc([C@H](NS(=O)(=O)C2CC2)C(F)(F)F)c2ccc(-c3c(F)cccc3Cl)cc21]

The title compound was prepared following the same general protocol as described for Step 5, Example 1, using 2-bromo-1-chloro-3-fluorobenzene and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.81 (d, J=8.0 Hz, 1H), 7.36 (s, 1H), 7.33-7.27 (m, 3H), 7.12 (d, J=8.0 Hz, 1H), 7.08 (t, J=8.0 Hz, 1H), 5.46 (q, J=8.0 Hz, 1H), 5.26 (J=8.0 Hz, 1H), 3.85 (s, 2H), 2.49-2.42 (m, 1H), 1.24-1.11 (m, 2H), 1.00 (s, 9H), 0.95-0.90 (m, 2H)

### Example 465: N-[(1S)-1-[1-(2,2-dimethylpropyl)-5-fluoro-6-thiazol-4-yl-indol-3-yl]-2,2,2-trifluoro-ethyl]cyclopropanesulfonamide (465)

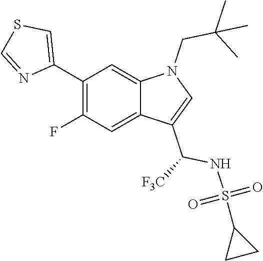 [SMILES: CC(C)(C)Cn1cc([C@H](NS(=O)(=O)C2CC2)C(F)(F)F)c2cc(F)c(-c3cscn3)cc21]

A mixture of N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (137 mg, 257.33 umol), 4-bromothiazole (84.42 mg, 514.66 umol, 45.88 uL) and K2CO3 (98.40 mg, 711.99 umol) in dioxane (3 mL) and water (0.7 mL) was stirred at 80° C. overnight under Nitrogen. The mixture was concentrated, the residue was added water, the aqueous phase was extracted with EtOAc twice. the combined organic phases were were washed with brine, dried with anhydrous Na2SO4 and filtered. The filtrate was concentrated to get oil, which was purified by silica gel plate to get N-[(1S)-1-[1-(2,2-dimethylpropyl)-5-fluoro-6-thiazol-4-yl-indol-3-yl]-2,2,2-trifluoro-ethyl]cyclopropanesulfonamide (39.22 mg, 80.11 umol) as a white solid. ¹H NMR (400 MHz, CDCl₃) δ 8.82 (d, J=1.7 Hz, 1H), 8.18 (d, J=6.1 Hz, 1H), 7.77 (t, J=2.2 Hz, 1H), 7.42 (d, J=12.2 Hz, 1H), 7.17 (s, 1H), 5.36-5.21 (m, 1H), 5.01 (d, J=8.1 Hz, 1H), 3.88 (s, 2H), 2.36 (ddd, J=8.0, 4.7, 3.2 Hz, 1H), 1.17-1.01 (m, 2H), 0.98-0.75 (m, 11H). LCMS: 489.8 m/z [M+H]⁺.

### Example 466: (S)—N-(1-(6-(2-cyanopyridin-3-yl)-1-neopentyl-1H-indol-3-yl)-2,2,2-trifluoroethyl)cyclopropanesulfonamide (466)

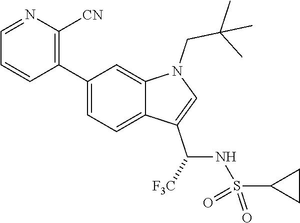 [SMILES: CC(C)(C)Cn1cc([C@H](NS(=O)(=O)C2CC2)C(F)(F)F)c2ccc(-c3cccnc3C#N)cc21]

The title compound was prepared following the same general protocol as described for Step 5, Example 1, using 3-bromopicolinonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. ¹HNMR (CDCl₃, 400 MHz) δ 8.58 (d, J=8.0 Hz, 1H), 7.81 (d, J=8.0 Hz, 1H), 7.78 (d, J=8.0 Hz, 1H), 7.52-7.49 (m, 2H), 7.25 (s, 1H), 7.21 (d, J=8.0 Hz, 1H), 5.69 (d, J=8.0 Hz, 1H), 5.37 (q, J=8.0 Hz, 1H), 3.79 (s, 2H), 2.39-2.33 (m, 1H), 1.26-1.10 (m, 2H), 0.96 (s, 9H), 0.89-0.79 (m, 2H)

### Example 467: (S)—N-(1-(6-(3-chloropyridin-2-yl)-1-neopentyl-1H-indol-3-yl)-2,2,2-trifluoroethyl)cyclopropanesulfonamide (467)

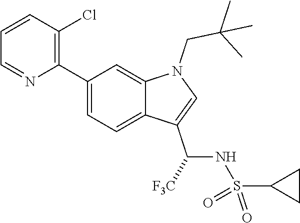 [SMILES: CC(C)(C)Cn1cc([C@H](NS(=O)(=O)C2CC2)C(F)(F)F)c2ccc(-c3ncccc3Cl)cc21]

The title compound was prepared following the same general protocol as described for Step 5, Example 1, using 2-bromo-3-chloropyridine and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 8.61 (d, J=4.0 Hz, 1H), 7.86-7.72 (m, 3H), 7.54 (d, J=8.0 Hz, 1H), 7.24-7.21 (m, 2H), 5.45 (q, J=8.0 Hz, 1H), 5.07 (d, J=8.0 Hz, 1H), 3.93 (s, 2H), 2.42-2.36 (m, 1H), 1.26-1.10 (m, 2H), 1.01 (s, 9H), 0.91-0.83 (m, 2H)

### Example 468: (S)—N-(1-(6-(3-cyanopyridin-2-yl)-1-neopentyl-1H-indol-3-yl)-2,2,2-trifluoroethyl)cyclopropanesulfonamide (468)

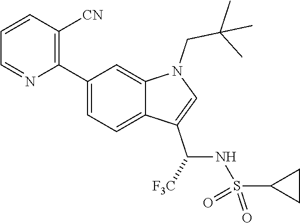 [SMILES: CC(C)(C)Cn1cc([C@H](NS(=O)(=O)C2CC2)C(F)(F)F)c2ccc(-c3ncccc3C#N)cc21]

The title compound was prepared following the same general protocol as described for Step 5, Example 1, using 2-chloronicotinonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 8.87 (d, J=4.0 Hz, 1H), 8.08 (dd, J=8.0 Hz, 4.0 Hz, 1H), 7.95 (s, 1H), 7.83 (d, J=8.0 Hz, 1H), 7.73 (d, J=8.0 Hz, 1H), 7.37-7.34 (m, 1H), 7.30 (s, 1H), 5.53 (d, J=8.0 Hz, 1H), 5.43 (q, J=8.0 Hz, 1H), 3.92 (d, J=4.0 Hz, 2H), 2.40-2.33 (m, 1H), 1.17-1.10 (m, 2H), 0.99 (s, 9H), 0.88-0.82 (m, 2H)

### Example 469: (S)—N-(2,2,2-trifluoro-1-(1-neopentyl-6-(pyridin-2-yl)-1H-indol-3-yl)ethyl)cyclopropanesulfonamide (469)

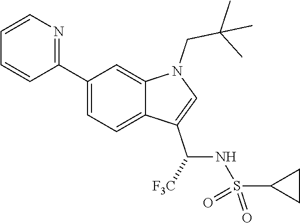 [SMILES: CC(C)(C)Cn1cc([C@H](NS(=O)(=O)C2CC2)C(F)(F)F)c2ccc(-c3ccccn3)cc21]

The title compound was prepared following the same general protocol as described in Example 382 (compound 382), using 2-(tributylstannyl)pyridine. ¹HNMR (CDCl₃, 400 MHz) δ 8.70 (d, J=4.0 Hz, 1H), 8.07 (s, 1H), 7.79-7.71 (m, 4H), 7.22-7.20 (m, 2H), 5.48-5.38 (m, 2H), 3.94 (s, 2H), 2.43-2.36 (m, 1H), 1.17-1.10 (m, 2H), 0.98 (s, 9H), 0.88-0.74 (m, 2H)

### Example 470: (S)—N-(1-(6-(3,5-dichloropyridin-4-yl)-1-neopentyl-1H-indol-3-yl)-2,2,2-trifluoroethyl)cyclopropanesulfonamide (470)

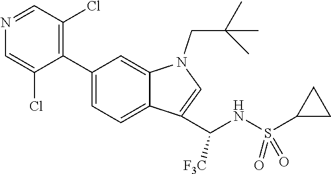 [SMILES: CC(C)(C)Cn1cc([C@H](NS(=O)(=O)C2CC2)C(F)(F)F)c2ccc(-c3c(Cl)cncc3Cl)cc21]

The title compound was prepared following the same general protocol as described for Step 5, Example 1, using 4-bromo-3,5-dichloropyridine and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 8.59 (s, 1H), 7.85 (d, J=4.0 Hz, 1H), 7.29-7.25 (m, 3H), 7.07 (d, J=8.0 Hz, 1H), 5.46 (q, J=8.0 Hz, 1H), 4.95 (d, J=8.0 Hz, 1H), 3.91 (s, 2H), 2.52-2.45 (m, 1H), 1.28-1.11 (m, 2H), 0.99-0.91 (m, 11H)

### Example 471: (S)-2-(3-(1-(cyclopropanesulfonamido)-2,2,2-trifluoroethyl)-1-neopentyl-1H-indol-6-yl)-N,N-dimethylbenzamide (471)

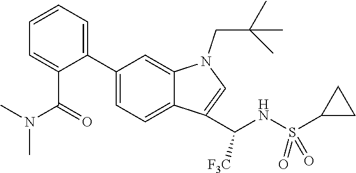 [SMILES: CN(C)C(=O)c1ccccc1-c1ccc2c([C@H](NS(=O)(=O)C3CC3)C(F)(F)F)cn(CC(C)(C)C)c2c1]

The title compound was prepared following the same general protocol as described for Step 5, Example 1, using 2-bromo-N,N-dimethylbenzamide and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.74 (d, J=8.0 Hz, 1H), 7.46-7.36 (m, 5H), 7.26-7.23 (m, 2H), 5.43 (q, J=8.0 Hz, 1H), 5.20 (d, J=8.0 Hz, 1H), 3.87 (d, J=8.0 Hz, 2H), 2.80 (s, 3H), 2.43-2.42 (m, 4H), 1.28-1.11 (m, 2H), 0.99-0.91 (m, 11H)

### Example 472: (S)—N-(1-(6-(2-cyano-4-fluorophenyl)-1-neopentyl-1H-indol-3-yl)-2,2,2-trifluoroethyl)cyclopropanesulfonamide (472)

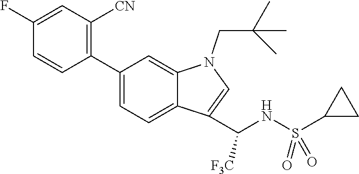 [SMILES: CC(C)(C)Cn1cc([C@H](NS(=O)(=O)C2CC2)C(F)(F)F)c2ccc(-c3ccc(F)cc3C#N)cc21]

The title compound was prepared following the same general protocol as described for Step 5, Example 1, using 2-bromo-5-fluorobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.81 (d, J=8.0 Hz, 1H), 7.53-7.51 (m, 2H), 7.45 (dd, J=8.0 Hz, 4.0 Hz, 1H), 7.36 (td, J=8.0 Hz, 4.0 Hz, 1H), 7.28 (s, 2H), 5.44 (q, J=8.0 Hz, 1H), 5.23 (d, J=8.0 Hz, 1H), 3.89 (s, 2H), 2.46-2.40 (m, 1H), 1.28-1.11 (m, 2H), 1.02 (s, 9H), 0.98-0.89 (m, 2H)

### Example 473: (S)—N-(1-(6-(3-cyano-2-fluorophenyl)-1-neopentyl-1H-indol-3-yl)-2,2,2-trifluoroethyl)cyclopropanesulfonamide (473)

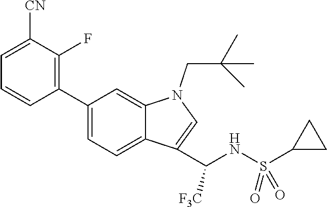 [SMILES: CC(C)(C)Cn1cc([C@H](NS(=O)(=O)C2CC2)C(F)(F)F)c2ccc(-c3cccc(C#N)c3F)cc21]

The title compound was prepared following the same general protocol as described for Step 5, Example 1, using 3-bromo-2-fluorobenzonitrile and (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide. HNMR (CDCl₃, 400 MHz) δ 7.81 (d, J=8.0 Hz, 1H), 7.73 (t, J=8.0 Hz, 1H), 7.60 (t, J=8.0 Hz, 1H), 7.51 (s, 1H), 7.42-7.27 (m, 3H), 5.43 (q, J=8.0 Hz, 1H), 5.36 (d, J=8.0 Hz, 1H), 3.88 (s, 2H), 2.46-2.40 (m, 1H), 1.28-1.11 (m, 2H), 0.99 (s, 9H), 0.98-0.84 (m, 2H)

### Example 474: N-[(1S)-2,2,2-trifluoro-1-[5-fluoro-1-(2-hydroxy-2-methyl-propyl)-6-[2-(trifluoromethyl)phenyl]indol-3-yl]ethyl]cyclopropanesulfonamide (474)

### Step 1: The synthesis of 1-(6-bromo-5-fluoro-1H-indol-1-yl)-2-methylpropan-2-ol

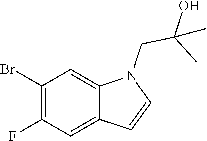 [SMILES: CC(C)(O)Cn1ccc2cc(F)c(Br)cc21]

6-bromo-5-fluoro-1H-indole (3 g, 14.02 mmol) in DMF (5 mL) was added to a suspension of NaH (1.12 g, 28.03 mmol, 60% purity) in anhydrous DMF (40 mL) in ice-water under nitrogen. The mixture was stirred for 1 h at room temperature, then cooling in ice-water for the addition of 2,2-dimethyloxirane (3.03 g, 42.05 mmol, 3.73 mL) and the resulting mixture was stirred at room temperature overnight under nitrogen. After quenching with water the reaction mixture was partitioned between ethyl acetate (50 mL) and water (50 mL). The aqueous layer was extracted using ethyl acetate (2*50 mL) and the combined organic layers were washed sequentially with sat. NH4Cl and brine, dried over anhydrous sodium sulfate. After the solvent was removed in vacuo, flash chromatography (EA/PE=0-80%) yielded 1-(6-bromo-5-fluoro-indol-1-yl)-2-methyl-propan-2-ol (4.15 g, 14.50 mmol) as yellow oil.

### Step 2: (S)—N-[(1S)-1-[6-bromo-5-fluoro-1-(2-hydroxy-2-methyl-propyl)indol-3-yl]-2,2,2-trifluoro-ethyl]-2-methyl-propane-2-sulfinamide

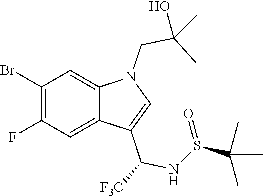 [SMILES: CC(C)(O)Cn1cc([C@H](N[S@@](=O)C(C)(C)C)C(F)(F)F)c2cc(F)c(Br)cc21]

The mixture of 1-(6-bromo-5-fluoro-indol-1-yl)-2-methyl-propan-2-ol (2.5 g, 8.74 mmol) in anhydrous CH2Cl2 (50 mL) was cooled to −10° C., BF3·OEt2 (2.48 g, 17.48 mmol, 2.16 mL) was added slowly, followed by addition of a solution of (S,E)-2-methyl-N-(2,2,2-trifluoroethylidene)propane-2-sulfinamide in DCM. The mixture was then continued to stir for 1-2 h. The mixture was diluted with DCM, followed by water. The aqueous layer was extracted with DCM twice. Organic layers were combined and washed with brine and concentrated in vacuo to obtain the crude (S)—N-[(1S)-1-[6-bromo-5-fluoro-1-(2-hydroxy-2-methyl-propyl)indol-3-yl]-2,2,2-trifluoro-ethyl]-2-methyl-propane-2-sulfinamide (3.29 g, 6.75 mmol).

### Step 3: 1-[3-[(1S)-1-amino-2,2,2-trifluoro-ethyl]-6-bromo-5-fluoro-indol-1-yl]-2-methyl-propan-2-ol

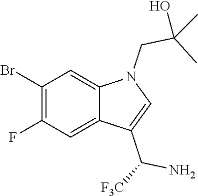 [SMILES: CC(C)(O)Cn1cc([C@H](N)C(F)(F)F)c2cc(F)c(Br)cc21]

A mixture of (S)—N-[(1S)-1-[6-bromo-5-fluoro-1-(2-hydroxy-2-methyl-propyl)indol-3-yl]-2,2,2-trifluoro-ethyl]-2-methyl-propane-2-sulfinamide (1.28 g, 2.63 mmol) in 4M HCl(g)/MeOH (15 mL) was stirred at room temperature for 3 hours. The mixture was concentrated in vacuo, the residue was basified to pH=8 with sat. NaHCO3, the aqueous phase was extracted with EtOAc twice. The combined organic phases were washed with brine, dried with anhydrous Na2SO4 and filtered. The filtrate was concentrated to give the crude product, which was purified by silica gel column (EA/PE=0-80%) to get 1-[3-[(1S)-1-amino-2,2,2-trifluoro-ethyl]-6-bromo-5-fluoro-indol-1-yl]-2-methyl-propan-2-ol (770 mg, 2.01 mmol, 76.51% yield) as light yellow oil.

### Step 4: 1-[3-[(1S)-1-amino-2,2,2-trifluoro-ethyl]-5-fluoro-6-[2-(trifluoromethyl)phenyl]indol-1-yl]-2-methyl-propan-2-ol

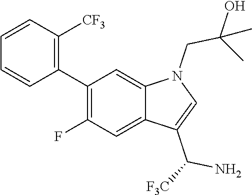 [SMILES: CC(C)(O)Cn1cc([C@H](N)C(F)(F)F)c2cc(F)c(-c3ccccc3C(F)(F)F)cc21]

A mixture of 1-[3-[(1S)-1-amino-2,2,2-trifluoro-ethyl]-6-bromo-5-fluoro-indol-1-yl]-2-methyl-propan-2-ol (770 mg, 2.01 mmol), [2-(trifluoromethyl)phenyl]boronic acid (496.28 mg, 2.61 mmol), Pd(dppf)Cl2 (441.22 mg, 0.603 mmol) and Na2CO3 (639.11 mg, 6.03 mmol) in dioxane (15 mL) and water (3 mL) was stirred at 80° C. overnight under Nitrogen. The mixture was added water and EtOAc, the mixture was stirred and separated, the aqueous phase was extracted with EtOAc twice, the combined organic phases were washed with brine and dried with Na2SO4, filtered and concentrated to get brown oil, which was purified by silica gel column (EA/PE=0-50%) to get 1-[3-[(1S)-1-amino-2,2,2-trifluoro-ethyl]-5-fluoro-6-[2-(trifluoromethyl)phenyl]indol-1-yl]-2-methyl-propan-2-ol (812 mg, 1.81 mmol) as brown oil.

The synthesis of N-[(1S)-2,2,2-trifluoro-1-[5-fluoro-1-(2-hydroxy-2-methyl-propyl)-6-[2-(trifluoromethyl)phenyl]indol-3-yl]ethyl]cyclopropanesulfonamide

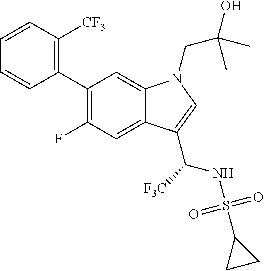 [SMILES: CC(C)(O)Cn1cc([C@H](NS(=O)(=O)C2CC2)C(F)(F)F)c2cc(F)c(-c3ccccc3C(F)(F)F)cc21]

A mixture of 1-[3-[(1S)-1-amino-2,2,2-trifluoro-ethyl]-5-fluoro-6-[2-(trifluoromethyl)phenyl]indol-1-yl]-2-methyl-propan-2-ol (130 mg, 289.93 μmol) and cyclopropanesulfonyl chloride (122.28 mg, 869.80 μmol, 88.61 uL) in pyridine (2 mL) was stirred at RT overnight. The mixture was added EtOAc and acidified to pH>1 with 1N HCl, then the aqueous was extracted with EtOAc twice. The combined organic phases were washed with brine twice, dried with Na2SO4, filtered and concentrated to get N-[(1S)-2,2,2-trifluoro-1-[5-fluoro-1-(2-hydroxy-2-methyl-propyl)-6-[2-(trifluoromethyl)phenyl]indol-3-yl]ethyl]cyclopropanesulfonamide (57.93 mg, 104.85 μmol, 36.16% yield), which was purified by prep-HPLC to get N-[(1S)-2,2,2-trifluoro-1-[5-fluoro-1-(2-hydroxy-2-methyl-propyl)-6-[2-(trifluoromethyl)phenyl]indol-3-yl]ethyl]cyclopropanesulfonamide (57.93 mg, 104.85 μmol, 36.16% yield) as yellow solids. ¹H NMR (400 MHz, DMSO) δ 8.51 (s, 1H), 7.86 (d, J=7.6 Hz, 1H), 7.70 (dq, J=22.9, 7.8 Hz, 4H), 7.54 (d, J=6.3 Hz, 1H), 7.46 (d, J=7.5 Hz, 1H), 5.49 (q, J=8.0 Hz, 1H), 4.61 (d, J=1.9 Hz, 1H), 4.07 (dd, J=42.2, 14.4 Hz, 2H), 2.42-2.25 (m, 1H), 1.17-0.52 (m, 10H). LCMS: 552.8 m/z [M+H]⁺.

### Example 475: N-[(1S)-2,2,2-trifluoro-1-[5-fluoro-1-(2-methoxy-2-methyl-propyl)-6-[2-(trifluoromethyl)phenyl]indol-3-yl]ethyl]cyclopropanesulfonamide (475)

### Step 1: 6-bromo-5-fluoro-1-(2-methoxy-2-methyl-propyl)indole

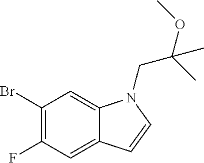 [SMILES: COC(C)(C)Cn1ccc2cc(F)c(Br)cc21]

1-(6-bromo-5-fluoro-indol-1-yl)-2-methyl-propan-2-ol (4 g, 13.98 mmol) was added to a suspension of NaH (1.96 g, 48.93 mmol, 60% purity) in anhydrous THF (50 mL) in ice-water under nitrogen. The mixture was stirred for 1 h at room temperature, then cooling in ice-water for the addition of iodomethane (13.89 g, 97.86 mmol, 6.09 mL) and the resulting mixture was stirred at room temperature overnight under nitrogen. After quenching with water, the aqueous phase was extracted with ethyl acetate three times. The combined organic layer was wash with sat. NH4Cl twice and brine, dried over anhydrous sodium sulfate. After the solvent was removed in vacuo, flash chromatography (EA/PE=0-50%) yielded 6-bromo-5-fluoro-1-(2-methoxy-2-methyl-propyl)indole (3.2 g, 10.66 mmol) as yellow oil.

### Step 2: N-[(1S)-2,2,2-trifluoro-1-[5-fluoro-1-(2-methoxy-2-methyl-propyl)-6-[2-(trifluoromethyl)phenyl]indol-3-yl]ethyl]cyclopropanesulfonamide

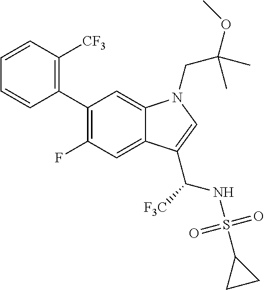 [SMILES: COC(C)(C)Cn1cc([C@H](NS(=O)(=O)C2CC2)C(F)(F)F)c2cc(F)c(-c3ccccc3C(F)(F)F)cc21]

A mixture of (1S)-2,2,2-trifluoro-1-[5-fluoro-1-(2-methoxy-2-methyl-propyl)-6-[2-(trifluoromethyl)phenyl]indol-3-yl]ethanamine (127 mg, 274.65 μmol, synthesized using the same method as Example 474, Step 4) and cyclopropanesulfonyl chloride (115.84 mg, 823.96 μmol, 83.94 uL) in pyridine (2 mL) was stirred at RT overnight. The mixture was added EtOAc and acidified to pH>1 with 1N HCl, then the aqueous was extracted with EtOAc twice. The combined organic phases were washed with brine twice, dried with Na2SO4, filtered and concentrated to get crude, which was purified by prep-HPLC to get N-[(1S)-2,2,2-trifluoro-1-[5-fluoro-1-(2-methoxy-2-methyl-propyl)-6-[2-(trifluoromethyl)phenyl]indol-3-yl]ethyl]cyclopropanesulfonamide (56.2 mg, 99.20 μmol) as a light-gray solid. ¹H NMR (400 MHz, DMSO) δ 8.65 (s, 1H), 7.86 (d, J=7.6 Hz, 1H), 7.70 (dq, J=11.0, 7.4 Hz, 4H), 7.56 (d, J=6.2 Hz, 1H), 7.47 (d, J=7.5 Hz, 1H), 5.50 (q, J=7.9 Hz, 1H), 4.30-4.03 (m, 2H), 3.13 (s, 3H), 2.37-2.21 (m, 1H), 1.20-0.53 (m, 10H). LCMS: 556.8 m/z [M+H]⁺.

### Example 476 The synthesis of 1-[[3-[(1S)-1-amino-2,2,2-trifluoro-ethyl]-5-fluoro-6-[2-(trifluoromethyl)phenyl]indol-1-yl]methyl]cyclopropanecarbonitrile (476)

### Step 1: (1-cyanocyclopropyl)methyl methanesulfonate

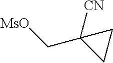 [SMILES: CS(=O)(=O)OCC1(C#N)CC1]

To a solution of methanesulfonyl chloride (884.66 mg, 7.72 mmol, 597.74 uL) 1-(hydroxymethyl)cyclopropanecarbonitrile (0.5 g, 5.15 mmol) in DCM (10 mL) and Triethylamine (1.56 g, 15.45 mmol, 2.15 mL) was added methanesulfonyl chloride (884.66 mg, 7.72 mmol, 597.74 uL) in portions under ice-bath. The mixture is stirred for 16 hours at room temperature, diluted with diethylether and washed with 1 M aqueous HCl solution and brine. After drying (MgSO4) the solvent is evaporated to give (1-cyanocyclopropyl)methyl methanesulfonate (900 mg, 5.14 mmol, 99.77% yield) as a yellow liquid.

### Step 2: The synthesis of 1-[(6-bromo-5-fluoro-indol-1-yl)methyl]cyclopropanecarbonitrile

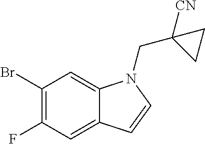 [SMILES: N#CC1(Cn2ccc3cc(F)c(Br)cc32)CC1]

6-bromo-5-fluoro-1H-indole (450 mg, 2.10 mmol), potassium iodide (698.03 mg, 4.20 mmol) in DMF (4 mL) was added Sodium hydride (201.82 mg, 8.41 mmol) under ice-bath. The mixture was heated to 50° C. and stirred for 15 min under N2. Then (1-cyanocyclopropyl)methyl methanesulfonate (736.73 mg, 4.20 mmol) in DMF (1 mL) was injected to reaction. The reaction was stirred at 80° C. overnight (16 h). TLC showed 6-bromo-5-fluoro-1H-indole was consumed. The mixture was cooled to RT and quenched with saturated NH4Cl and extracted with EA (50 mL×2). The combined organic layers were dried over anhydrous Na2SO4, filtered and concentrated. The residue obtained was purified by flash (PE/EA from 5% to 10%) to give 1-[(6-bromo-5-fluoro-indol-1-yl)methyl]cyclopropanecarbonitrile (1.2 g, 4.09 mmol, 194.71% yield) as a light yellow liquid.

### Step 2: The synthesis of (S)—N-[(1S)-1-[6-bromo-1-[(1-cyanocyclopropyl)methyl]-5-fluoro-indol-3-yl]-2,2,2-trifluoro-ethyl]-2-methyl-propane-2-sulfinamide

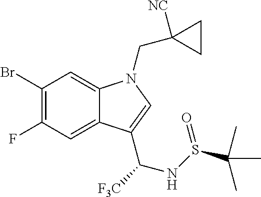 [SMILES: CC(C)(C)[S@](=O)N[C@@H](c1cn(CC2(C#N)CC2)c2cc(Br)c(F)cc12)C(F)(F)F]

The mixture of 1-[(6-bromo-5-fluoro-indol-1-yl)methyl]cyclopropanecarbonitrile (946.96 mg, 3.23 mmol) in DCM (15 mL) was cooled to −20° C., diethyloxonio(trifluoro)boranuide (596.04 mg, 4.20 mmol, 518.30 uL) was added slowly, followed by addition DCM solution of (NE,S)-2-methyl-N-(2,2,2-trifluoroethylidene)propane-2-sulfinamide (650 mg, 3.23 mmol). The mixture was then continued to stir at RT for 1.5 h and LCMS showed little product was observed. The mixture was diluted with DCM, followed by water. The aqueous layer was extracted with DCM twice. Organic layers were combined and washed with brine and concentrated in vacuo to give the crude (S)—N-[(1S)-1-[6-bromo-1-[(1-cyanocyclopropyl)methyl]-5-fluoro-indol-3-yl]-2,2,2-trifluoro-ethyl]-2-methyl-propane-2-sulfinamide (1.2 g, 2.43 mmol) as a yellow liquid. It was used in next step directly.

### Step 4: The synthesis of (1-[[3-[(1S)-1-amino-2,2,2-trifluoro-ethyl]-6-bromo-5-fluoro-indol-1-yl]methyl]cyclopropanecarbonitrile

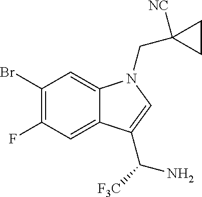 [SMILES: N#CC1(Cn2cc([C@H](N)C(F)(F)F)c3cc(F)c(Br)cc32)CC1]

To a (S)—N-[(1S)-1-[6-bromo-1-[(1-cyanocyclopropyl)methyl]-5-fluoro-indol-3-yl]-2,2,2-trifluoro-ethyl]-2-methyl-propane-2-sulfinamide (900 mg, 1.82 mmol) was added 4 M HCl/MeOH (10 mL) at RT. The mixture was stirred at RT for 1 h. LCMS showed the reaction was completed. The reaction was concentrated and adjusted pH to 7 by addition saturated Na2CO3. The aqueous was extracted with DCM (50 mL×2). The combined organic layers were dried over anhydrous Na2SO₄, filtered and concentrated. The residue obtained was purified by Flash to give 1-[[3-[(1S)-1-amino-2,2,2-trifluoro-ethyl]-6-bromo-5-fluoro-indol-1-yl]methyl]cyclopropanecarbonitrile (620 mg, 1.59 mmol, 87.28% yield) as a yellow oil.

### Step 5: The synthesis of 1-[[3-[(1S)-1-amino-2,2,2-trifluoro-ethyl]-5-fluoro-6-[2-(trifluoromethyl)phenyl]indol-1-yl]methyl]cyclopropanecarbonitrile

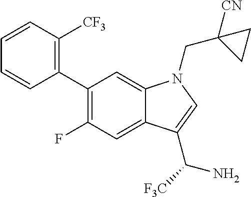 [SMILES: N#CC1(Cn2cc([C@H](N)C(F)(F)F)c3cc(F)c(-c4ccccc4C(F)(F)F)cc32)CC1]

A mixture of 1-[[3-[(1S)-1-amino-2,2,2-trifluoro-ethyl]-6-bromo-5-fluoro-indol-1-yl]methyl]cyclopropanecarbonitrile (620 mg, 1.59 mmol), [2-(trifluoromethyl)phenyl]boronic acid (392.34 mg, 2.07 mmol), cyclopentyl(diphenyl)phosphane;dichloropalladium;iron (348.81 mg, 476.71 umol) and Sodium carbonate (505.26 mg, 4.77 mmol, 199.71 uL) in dioxane (5 mL) was stirred at 80° C. overnight under Nitrogen. Lcms showed the reaction was completed. The mixture was concentrated, the residue was purified by silica gel column (EA/PE=0-50%) to get 1-[[3-[(1S)-1-amino-2,2,2-trifluoro-ethyl]-5-fluoro-6-[2-(trifluoromethyl)phenyl]indol-1-yl]methyl]cyclopropanecarbonitrile (550 mg, 1.21 mmol, 76.01% yield) as a yellow oil.

### Step 6: The synthesis of 1-[[3-[(1S)-1-amino-2,2,2-trifluoro-ethyl]-5-fluoro-6-[2-(trifluoromethyl)phenyl]indol-1-yl]methyl]cyclopropanecarbonitrile

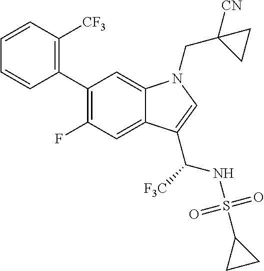 [SMILES: N#CC1(Cn2cc([C@H](NS(=O)(=O)C3CC3)C(F)(F)F)c3cc(F)c(-c4ccccc4C(F)(F)F)cc32)CC1]

To a mixture of 1-[[3-[(1S)-1-amino-2,2,2-trifluoro-ethyl]-5-fluoro-6-[2-(trifluoromethyl)phenyl]indol-1-yl]methyl]cyclopropanecarbonitrile (100 mg, 219.60 umol) in pyridine (3 mL) at RT was added cyclopropanesulfonyl chloride (92.62 mg, 658.80 umol, 67.12 uL). The mixture was stirred at RT overnight and the completion of reaction was monitored by lcms. The mixture was concentrated and diluted with EtOAc, washed with 2N HCl, water, Sat's NaHCO3 and brine and dried over Na2SO4. Solvent was removed in vacuo to obtain the crude which was purified by chromatography on silica gel (EtOAc/hex) to afford N-[(1S)-1-[1-[(1-cyanocyclopropyl)methyl]-5-fluoro-6-[2-(trifluoromethyl)phenyl]indol-3-yl]-2,2,2-trifluoro-ethyl]cyclopropanesulfonamide (40 mg, 71.49 umol, 32.56% yield) as a white solid. ¹H NMR (400 MHz, MeOD) δ 7.81 (d, J=7.6 Hz, 1H), 7.73 (s, 1H), 7.66 (t, J=7.3 Hz, 1H), 7.57 (dd, J=19.8, 9.1 Hz, 2H), 7.45 (dd, J=9.4, 7.0 Hz, 2H), 5.42 (q, J=7.9 Hz, 1H), 4.57-4.46 (m, 1H), 4.24 (dd, J=15.2, 4.5 Hz, 1H), 2.37-2.23 (m, 1H), 1.34 (dd, J=12.2, 7.9 Hz, 2H), 1.27-1.18 (m, 2H), 1.04-0.93 (m, 2H), 0.75 (dt, J=23.0, 15.3 Hz, 2H). LCMS. 576.7 m/z [M+H₂O]⁺.

### Example 477: N-[(1S)-1-[1-(2,2-dimethylpropyl)-5-fluoro-6-tetralin-5-yl-indol-3-yl]-2,2,2-trifluoro-ethyl]cyclopropanesulfonamide (477)

### Step 1: tetralin-5-yl trifluoromethanesulfonate

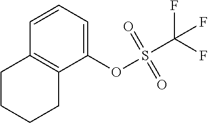 [SMILES: O=S(=O)(Oc1cccc2c1CCCC2)C(F)(F)F]

Tetralin-5-ol (500 mg, 3.37 mmol) and N,N-diethylethanamine (512.09 mg, 5.06 mmol, 705.36 uL) were dissolved in DCM (5 mL). trifluoromethylsulfonyl trifluoromethanesulfonate (1.14 g, 4.05 mmol, 679.91 uL) was added at ice-bath, and the mixture was stirred at RT for 1.5 h. The mixture was poured into water (50 mL) and extracted three times with DCM (50 mL). The combined organic layers were dried (MgSO4), and concentrated. The residue obtained was purified by column chromatography of silica gel eluting with PE/EA (10/1) to give tetralin-5-yl trifluoromethanesulfonate (900 mg, 3.21 mmol, 95.18% yield) as light-yellow oil.

### Step 2: N-[(1S)-1-[1-(2,2-dimethylpropyl)-5-fluoro-6-tetralin-5-yl-indol-3-yl]-2,2,2-trifluoro-ethyl]cyclopropanesulfonamide

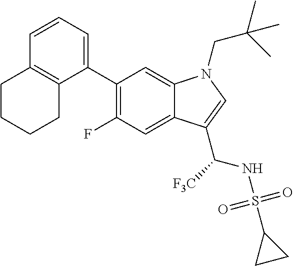 [SMILES: CC(C)(C)Cn1cc([C@H](NS(=O)(=O)C2CC2)C(F)(F)F)c2cc(F)c(-c3cccc4c3CCCC4)cc21]

Tetralin-5-yl trifluoromethanesulfonate (150.00 mg, 535.21 umol), N-[(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethyl]cyclopropanesulfonamide (316.61 mg, 356.81 umol), palladium;triphenylphosphane (82.46 mg, 71.36 umol) and disodium;carbonate (113.45 mg, 1.07 mmol, 44.84 uL) were added to the sealed tube (20 ml). Then Dioxane (5 mL) and DCM (1 mL) were added to the mixture. The mixture was stirred at 80° C. for 2 h. LCMS showed desired product was observed. water (5 mL) was added to the mixture. The mixture was extracted with EA (50 ml). The organic layer was dried over anhydrous Na2SO4, filtered and concentrated. The residue obtained was purified by prep-HPLC to give N-[(1S)-1-[1-(2,2-dimethylpropyl)-5-fluoro-6-tetralin-5-yl-indol-3-yl]-2,2,2-trifluoro-ethyl]cyclopropanesulfonamide (35 mg, 64.83 umol, 18.17% yield, 99.4% purity) as a white solid. ¹H NMR (400 MHz, MeOD) δ 7.47 (d, J=10.6 Hz, 2H), 7.24 (d, J=6.0 Hz, 1H), 7.15-7.06 (m, 2H), 6.98 (d, J=6.9 Hz, 1H), 5.39 (q, J=7.9 Hz, 1H), 4.05-3.91 (m, 2H), 2.84 (d, J=4.5 Hz, 2H), 2.61-2.26 (m, 3H), 1.86-1.60 (m, 4H), 1.08-0.94 (m, 11H), 0.84-0.70 (m, 2H). LCMS: 536.8 m/z [M+H]⁺.

### Example 478: N-[(1S)-2,2,2-trifluoro-1-[5-fluoro-1-(2-hydroxy-2-methyl-propyl)-6-[2-(trifluoromethyl)phenyl]indol-3-yl]ethyl]cyclobutanesulfonamide (478)

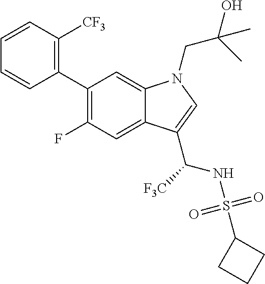 [SMILES: CC(C)(O)Cn1cc([C@H](NS(=O)(=O)C2CCC2)C(F)(F)F)c2cc(F)c(-c3ccccc3C(F)(F)F)cc21]

A mixture of 1-[3-[(1S)-1-amino-2,2,2-trifluoro-ethyl]-5-fluoro-6-[2-(trifluoromethyl)phenyl]indol-1-yl]-2-methyl-propan-2-ol (80 mg, 178.42 μmol) and cyclobutanesulfonyl chloride (165.44 mg, 1.07 mmol) in pyridine (0.4 mL) was stirred at 100° C. for 2 hours in the microwave. The reaction was concentrated, the residue was diluted with EtOAc and 1N HCl, the aqueous phase was separated and extracted with EtOAc twice, the combined organic phases were wash with brine and dried with Na₂SO₄, filtered and concentrated to get black oil, which was purified by prep-HPLC to get N-[(1S)-2,2,2-trifluoro-1-[5-fluoro-1-(2-hydroxy-2-methyl-propyl)-6-[2-(trifluoromethyl)phenyl]indol-3-yl]ethyl]cyclobutanesulfonamide (13.26 mg, 23.41 μmol) as a light yellow solid. ¹H NMR (400 MHz, CDCl₃) δ 7.78 (d, J=7.5 Hz, 1H), 7.58 (t, J=7.2 Hz, 1H), 7.52 (t, J=7.6 Hz, 1H), 7.46 (d, J=9.9 Hz, 1H), 7.38 (d, J=8.7 Hz, 2H), 7.28 (s, 1H), 5.33 (q, J=7.6 Hz, 1H), 4.02 (s, 2H), 3.84 (s, 1H), 2.59-2.38 (m, 2H), 2.24 (s, 2H), 2.06-1.87 (m, 2H), 1.65-1.55 (m, 2H), 1.27-1.18 (m, 6H). LCMS: 583.8 m/z [M+H]⁺.

### Example 479: N-[(1S)-1-[1-(2,2-dimethylpropyl)-5-fluoro-6-tetralin-5-yl-indol-3-yl]-2,2,2-trifluoro-ethyl]cyclobutanesulfonamide (479)

### Step 1: The synthesis of (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine

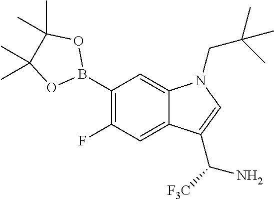 [SMILES: CC(C)(C)Cn1cc([C@H](N)C(F)(F)F)c2cc(F)c(B3OC(C)(C)C(C)(C)O3)cc21]

A mixture of (1S)-1-[6-bromo-1-(2,2-dimethylpropyl)-5-fluoro-indol-3-yl]-2,2,2-trifluoro-ethanamine (500 mg, 1.31 mmol), 4,4,5,5-tetramethyl-2-(4,4,5,5-tetramethyl-1,3,2-dioxaborolan-2-yl)-1,3,2-dioxaborolane (333.07 mg, 1.31 mmol) cyclopentyl(diphenyl)phosphane;dichloropalladium;iron (287.92 mg, 393.49 umol) and potassium;acetate (386.18 mg, 3.93 mmol, 245.97 uL) in dioxane (10 mL) was stirred at 80° C. overnight under Nitrogen. The completion of reaction was monitored by lcms. The mixture was concentrated, the residue was purified by silica gel column (EA/PE=0-25%) to get (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (680 mg, 1.11 mmol, 84.74% yield) as yellow oil.

### Step 2: (1S)-1-[1-(2,2-dimethylpropyl)-5-fluoro-6-tetralin-5-yl-indol-3-yl]-2,2,2-trifluoro-ethanamine

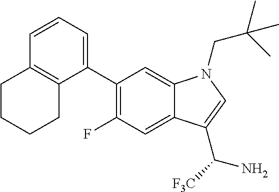 [SMILES: CC(C)(C)Cn1cc([C@H](N)C(F)(F)F)c2cc(F)c(-c3cccc4c3CCCC4)cc21]

Tetralin-5-yl trifluoromethanesulfonate (200 mg, 713.62 umol), (1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (305.62 mg, 713.62 umol), disodium;carbonate (151.27 mg, 1.43 mmol, 59.79 uL) and palladium;triphenylphosphane (164.93 mg, 142.72 umol) were added to the sealed tube (20 ml). Then dioxane (4 mL) and H2O (1 mL) were added to the mixture. The mixture was stirred at 85° C. for 2 h. LCMS showed desired product was observed. water (5 mL) was added to the mixture. The mixture was extracted with EA (50 ml). The organic layer was dried over anhydrous Na₂SO₄, filtered and concentrated. The residue obtained was purified by flash to give (1S)-1-[1-(2,2-dimethylpropyl)-5-fluoro-6-tetralin-5-yl-indol-3-yl]-2,2,2-trifluoro-ethanamine (250 mg, 578.04 umol).

### Step 3: N-[(1S)-1-[1-(2,2-dimethylpropyl)-5-fluoro-6-tetralin-5-yl-indol-3-yl]-2,2,2-trifluoro-ethyl]cyclobutanesulfonamide

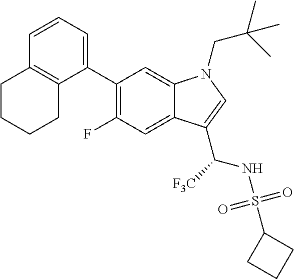

To a mixture of (1S)-1-[1-(2,2-dimethylpropyl)-5-fluoro-6-tetralin-5-yl-indol-3-yl]-2,2,2-trifluoro-ethanamine (100 mg, 231.22 umol) in pyridine (5 mL) at RT was added cyclobutanesulfonyl chloride (89.37 mg, 462.43 umol). The mixture was stirred at RT overnight and the completion of reaction was monitored by lcms. The mixture was concentrated and diluted with EtOAc, washed with 2N HCl, water, Sat'd NaHCO₃ and brine and dried over Na₂SO₄. Solvent was removed in vacuo to obtain the crude which was purified by chromatography on silica gel (EtOAc/hex) to afford N-[(1S)-1-[1-(2,2-dimethylpropyl)-5-fluoro-6-tetralin-5-yl-indol-3-yl]-2,2,2-trifluoro-ethyl]cyclobutanesulfonamide (3.3 mg, 5.99 umol, 2.59% yield) as a white solid. ¹H NMR (400 MHz, MeOD) δ 7.46 (t, J=5.2 Hz, 2H), 7.24 (d, J=6.0 Hz, 1H), 7.15-7.06 (m, 2H), 6.99 (d, J=6.5 Hz, 1H), 5.39-5.28 (m, 1H), 4.56 (s, 1H), 3.98 (t, J=4.8 Hz, 2H), 3.74-3.64 (m, 1H), 2.84 (d, J=4.2 Hz, 2H), 2.53 (s, 1H), 2.49-2.28 (m, 3H), 2.11-1.97 (m, 2H), 1.92-1.67 (m, 5H), 0.98 (s, 9H). LCMS: 550.8 m/z [M+H]⁺.

### Example 480: (S)—N-(2,2,2-trifluoro-1-(1-pivaloyl-6-(2-(trifluoromethyl)phenyl)-1H-indol-3-yl)ethyl)cyclopropanesulfonamide (480)

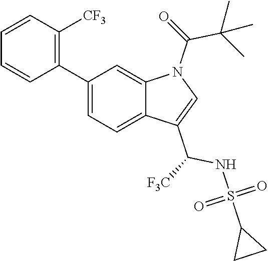 [SMILES: CC(C)(C)C(=O)n1cc([C@H](NS(=O)(=O)C2CC2)C(F)(F)F)c2ccc(-c3ccccc3C(F)(F)F)cc21]

### Step 1: 6-bromo-1-(4-methoxybenzyl)-1H-indole

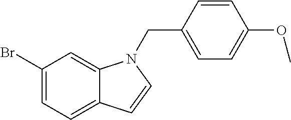 [SMILES: COc1ccc(Cn2ccc3ccc(Br)cc32)cc1]

The title compound was prepared following the same general protocol as described for Example 34, using 6-bromo-1H-indole and 1-(chloromethyl)-4-methoxybenzene. ESI-MS (m/z): 315.56, 317.43 [M+1]⁺.

### Step 2: (S)—N—((S)-1-(6-bromo-1-(4-methoxybenzyl)-1H-indol-3-yl)-2,2,2-trifluoroethyl)-2-methylpropane-2-sulfinamide

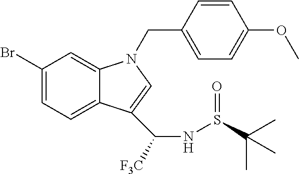 [SMILES: COc1ccc(Cn2cc([C@H](N[S@@](=O)C(C)(C)C)C(F)(F)F)c3ccc(Br)cc32)cc1]

The title compound was prepared following the same general protocol as described for Step 3, Example 116, using (S)-2-methyl-N-(3,3,3-trifluoroprop-1-en-1-yl)propane-2-sulfinamide and 6-bromo-1-(4-methoxybenzyl)-1H-indole. ESI-MS (m/z): 516.58, 518.44 [M+1]⁺.

### Step 3: (S)-1-(6-bromo-1-(4-methoxybenzyl)-1H-indol-3-yl)-2,2,2-trifluoroethan-1-amine

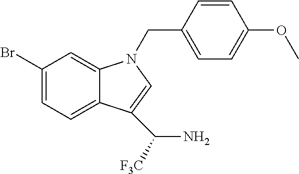 [SMILES: COc1ccc(Cn2cc([C@H](N)C(F)(F)F)c3ccc(Br)cc32)cc1]

The title compound was prepared following the same general protocol as described for Step 4, Example 116, using (S)—N—((S)-1-(6-bromo-1-(4-methoxybenzyl)-1H-indol-3-yl)-2,2,2-trifluoroethyl)-2-methylpropane-2-sulfinamide.

### Step 4: (S)—N-(1-(6-bromo-1-(4-methoxybenzyl)-1H-indol-3-yl)-2,2,2-trifluoroethyl)cyclopropanesulfonamide

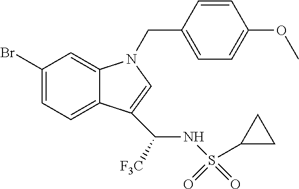 [SMILES: COc1ccc(Cn2cc([C@H](NS(=O)(=O)C3CC3)C(F)(F)F)c3ccc(Br)cc32)cc1]

The title compound was prepared following the same general protocol as described for Step 5, Example 116, using (S)-1-(6-bromo-1-(4-methoxybenzyl)-1H-indol-3-yl)-2,2,2-trifluoroethan-1-amine. ESI-MS (m/z): 516.67, 518.47 [M+1]⁺.

### Step 5: (S)—N-(2,2,2-trifluoro-1-(1-(4-methoxybenzyl)-6-(2-(trifluoromethyl)phenyl)-1H-indol-3-yl)ethyl)cyclopropanesulfonamide

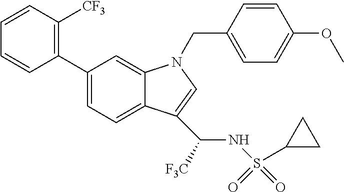 [SMILES: COc1ccc(Cn2cc([C@H](NS(=O)(=O)C3CC3)C(F)(F)F)c3ccc(-c4ccccc4C(F)(F)F)cc32)cc1]

The title compound was prepared following the same general protocol as described for Step 5, Example 1, using (S)—N-(1-(6-bromo-1-(4-methoxybenzyl)-1H-indol-3-yl)-2,2,2-trifluoroethyl)cyclopropanesulfonamide and (2-(trifluoromethyl)phenyl)boronic acid. ESI-MS (m/z): 582.57 [M+1]⁺.

### Step 6: (S)—N-(2,2,2-trifluoro-1-(6-(2-(trifluoromethyl)phenyl)-1H-indol-3-yl)ethyl)cyclopropanesulfonamide

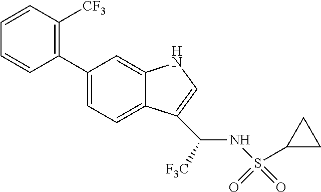 [SMILES: O=S(=O)(N[C@@H](c1c[nH]c2cc(-c3ccccc3C(F)(F)F)ccc12)C(F)(F)F)C1CC1]

To a solution of (S)—N-(2,2,2-trifluoro-1-(1-(4-methoxybenzyl)-6-(2-(trifluoromethyl)phenyl)-1H-indol-3-yl)ethyl)cyclopropanesulfonamide (0.1656 g, 0.582 mmmol) in toluene (10 mL) and H₂O (0.3 mL) was added DDQ (0.291 g, 1.28 mmol). The mixture was stirred for 3 days in an 80° C. oil bath. The mixture was cooled to rt, diluted with EtOAc, and washed with saturated NaHCO₃, brine, dried (Na₂SO₄) and filtered. The solvent was removed and the crude was purified by silica gel to obtain the title compound. ESI-MS (m/z): 462.78 [M+1]⁺.

### Step 7: (S)—N-(2,2,2-trifluoro-1-(1-pivaloyl-6-(2-(trifluoromethyl)phenyl)-1H-indol-3-yl)ethyl)cyclopropanesulfonamide

The title compound was prepared following the same general protocol as described for Step 4, Example B-285, using (S)—N-(2,2,2-trifluoro-1-(6-(2-(trifluoromethyl)phenyl)-1H-indol-3-yl)ethyl)cyclopropanesulfonamide. ¹HNMR (CDCl₃, 400 MHz) δ 8.53 (s, 1H), 7.93 (s, 1H), 7.74 (t, J=8.0 Hz, 2H), 7.55 (t, J=8.0 Hz, 1H), 7.47 (t, J=8.0 Hz, 1H), 7.34 (t, J=8.0 Hz, 2H), 5.45 (q, J=8.0 Hz, 1H), 5.31 (d, J=8.0 Hz, 1H), 2.45-2.40 (m, 1H), 1.26 (s, 9H), 1.28-1.11 (m, 2H), 0.98-0.91 (m, 2H)

### Example 481: N-(2-(6-(2,4-bis(trifluoromethyl)phenyl)-1-neopentyl-1H-indol-3-yl)ethyl)cyclopropanesulfonamide (481)

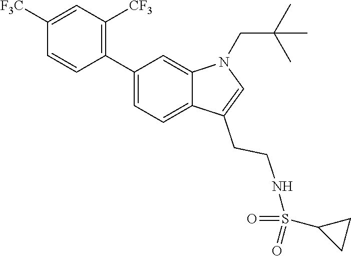 [SMILES: CC(C)(C)Cn1cc(CCNS(=O)(=O)C2CC2)c2ccc(-c3ccc(C(F)(F)F)cc3C(F)(F)F)cc21]

The title compound was prepared following the same general protocol as described for Step 5, Example 1, using N-(2-(6-bromo-1-neopentyl-1H-indol-3-yl)ethyl)cyclopropanesulfonamide and (2,4-bis(trifluoromethyl)phenyl)boronic acid. ESI-MS (m/z): 546.78 [M+1]⁺.

### Example 482: N-(2-(6-(4-chloro-2-cyanophenyl)-1-neopentyl-1H-indol-3-yl)ethyl)cyclopropanesulfonamide (482)

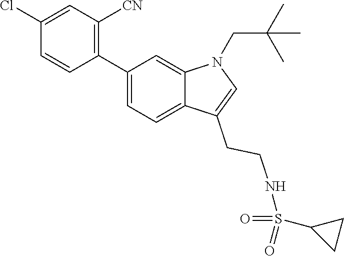 [SMILES: CC(C)(C)Cn1cc(CCNS(=O)(=O)C2CC2)c2ccc(-c3ccc(Cl)cc3C#N)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-5-chlorobenzonitrile. ESI-MS (m/z): 470.64 [M+1]⁺.

### Example 483: N-(2-(6-(2,5-bis(trifluoromethyl)phenyl)-1-neopentyl-1H-indol-3-yl)ethyl)cyclopropanesulfonamide (483)

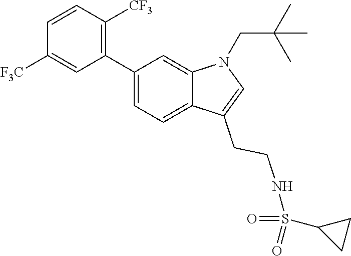 [SMILES: CC(C)(C)Cn1cc(CCNS(=O)(=O)C2CC2)c2ccc(-c3cc(C(F)(F)F)ccc3C(F)(F)F)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-1,4-bis(trifluoromethyl)benzene. ESI-MS (m/z): 546.78 [M+1]⁺.

### Example 484: N-(2-(6-(2-chloro-5-(trifluoromethyl)phenyl)-1-neopentyl-1H-indol-3-yl)ethyl)cyclopropanesulfonamide (484)

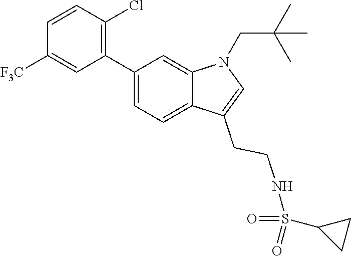 [SMILES: CC(C)(C)Cn1cc(CCNS(=O)(=O)C2CC2)c2ccc(-c3cc(C(F)(F)F)ccc3Cl)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-1-chloro-4-(trifluoromethyl)benzene. ESI-MS (m/z): 512.75 [M+1]⁺.

### Example 485: N-(2-(6-(2-chloro-3-(trifluoromethyl)phenyl)-1-neopentyl-1H-indol-3-yl)ethyl)cyclopropanesulfonamide (485)

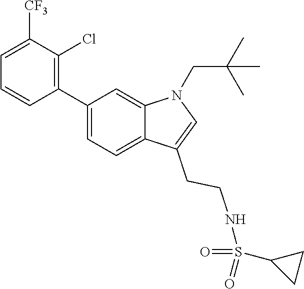 [SMILES: CC(C)(C)Cn1cc(CCNS(=O)(=O)C2CC2)c2ccc(-c3cccc(C(F)(F)F)c3Cl)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 1-bromo-2-chloro-3-(trifluoromethyl)benzene. ESI-MS (m/z): 512.73 [M+1]⁺.

### Example 486: N-(2-(6-(2-chloro-3-cyanophenyl)-1-neopentyl-1H-indol-3-yl)ethyl)cyclopropanesulfonamide (486)

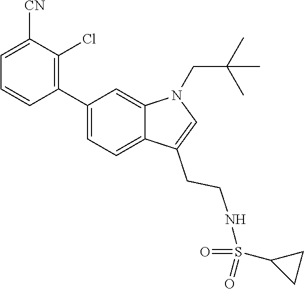 [SMILES: CC(C)(C)Cn1cc(CCNS(=O)(=O)C2CC2)c2ccc(-c3cccc(C#N)c3Cl)cc21]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 3-bromo-2-chlorobenzonitrile. ESI-MS (m/z): 469.73 [M+1]⁺.

### Example 487: N-(2-(5-(2-cyano-4-(trifluoromethyl)phenyl)-3-neopentyl-1H-indol-1-yl)ethyl)cyclopropanesulfonamide (487)

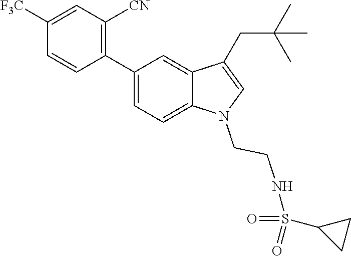 [SMILES: CC(C)(C)Cc1cn(CCNS(=O)(=O)C2CC2)c2ccc(-c3ccc(C(F)(F)F)cc3C#N)cc12]

The title compound was prepared following the same general protocol as described for Step 5, Example 1, using N-(2-(1-neopentyl-6-(4,4,5,5-tetramethyl-1,3,2-dioxaborolan-2-yl)-1H-indol-3-yl)ethyl)cyclopropanesulfonamide and 2-bromo-5-(trifluoromethyl)benzonitrile. ESI-MS (m/z): 503.86 [M+1]⁺.

### Example 488: N-(2-(5-(2,4-dichlorophenyl)-3-neopentyl-1H-indol-1-yl)ethyl)cyclopropanesulfonamide (488)

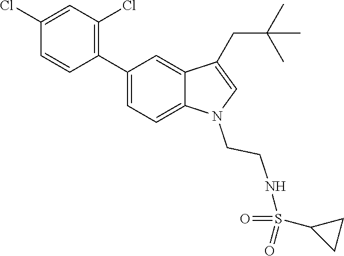 [SMILES: CC(C)(C)Cc1cn(CCNS(=O)(=O)C2CC2)c2ccc(-c3ccc(Cl)cc3Cl)cc12]

The title compound was prepared following the same general protocol as described for Step 5, Example 1, using N-(2-(5-bromo-3-neopentyl-1H-indol-1-yl)ethyl)cyclopropanesulfonamide and (2,4-dichlorophenyl)boronic acid. ESI-MS (m/z): 478.66 [M+1]⁺.

### Example 489: N-[(1S)-2,2,2-trifluoro-1-[5-fluoro-1-[(3-methyloxetan-3-yl)methyl]-6-[2-(trifluoromethyl)phenyl]indol-3-yl]ethyl]cyclopropanesulfonamide (489)

### Step 1: (S)—N-[(1S)-1-(6-bromo-5-fluoro-1H-indol-3-yl)-2,2,2-trifluoro-ethyl]-2-methyl-propane-2-sulfinamide

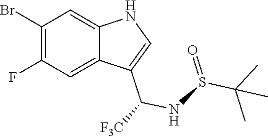

The mixture of 6-bromo-5-fluoro-1H-indole (2 g, 9.34 mmol) in anhydrous CH₂Cl₂ (20 mL) was cooled to −10° C., BF₃·OEt₂ (2.07 g, 14.57 mmol, 1.80 mL) was added slowly, followed by addition of imine mixture. The mixture was then continued to stir for 1-2 h and the completion of reaction was monitored by TLC indicating the complete consume of indole. The mixture was diluted with DCM, followed by water. The aqueous layer was extracted with DCM twice. Organic layers were combined and washed with brine and concentrated in vacuo to obtain the crude, which was purified by silica gel column (EA/PE=0-70%) to get (S)—N-[(1S)-1-(6-bromo-5-fluoro-1H-indol-3-yl)-2,2,2-trifluoro-ethyl]-2-methyl-propane-2-sulfinamide (2.52 g, 6.07 mmol)

### Step 2: The synthesis of (1S)-1-(6-bromo-5-fluoro-1H-indol-3-yl)-2,2,2-trifluoro-ethanamine

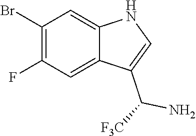 [SMILES: N[C@@H](c1c[nH]c2cc(Br)c(F)cc12)C(F)(F)F]

A mixture of(S)—N-[(1S)-1-(6-bromo-5-fluoro-1H-indol-3-yl)-2,2,2-trifluoro-ethyl]-2-methyl-propane-2-sulfinamide (690 mg, 1.66 mmol) in 4M HCl(g)/MeOH (10 mL) was stirred at RT for 1.5 hours, the mixture was concentrated, the residue was basified to PH=8 with Sat. NaHCO₃, the aqueous phase was extracted with EtOAc twice, the combined organic phases were washed with brine and concentrated to get (1S)-1-(6-bromo-5-fluoro-1H-indol-3-yl)-2,2,2-trifluoro-ethanamine (440 mg, 1.41 mmol) as a light yellow solid.

### Step 3: The synthesis of (1S)-2,2,2-trifluoro-1-[5-fluoro-6-[2-(trifluoromethyl)phenyl]-1H-indol-3-yl]ethanamine

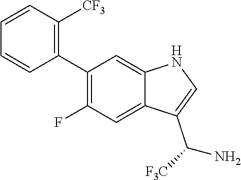 [SMILES: N[C@@H](c1c[nH]c2cc(-c3ccccc3C(F)(F)F)c(F)cc12)C(F)(F)F]

A mixture of (1S)-1-(6-bromo-5-fluoro-1H-indol-3-yl)-2,2,2-trifluoro-ethanamine (440 mg, 1.41 mmol), [2-(trifluoromethyl)phenyl]boronic acid (347.57 mg, 1.83 mmol) and Na₂CO₃ (448.33 mg, 4.23 mmol) in dioxane (9 mL) and water (2 mL) was stirred at 80° C. for 4 hours under Nitrogen. The completion of reaction was monitored by lcms. The mixture was added water and EtOAc, the mixture was stirred and separated, the aqueous phase was extracted with EtOAc twice, the combined organic phases were washed with brine and dried with Na2SO4, filtered and concentrated to get brown oil, which was purified by silica gel column (EA/PE=0-50%) to get (1S)-2,2,2-trifluoro-1-[5-fluoro-6-[2-(trifluoromethyl)phenyl]-1H-indol-3-yl]ethanamine (474 mg, 1.26 mmol) as brown oil.

### Step 4: The synthesis of N-[(1S)-2,2,2-trifluoro-1-[5-fluoro-6-[2-(trifluoromethyl)phenyl]-1H-indol-3-yl]ethyl]cyclopropanesulfonamide

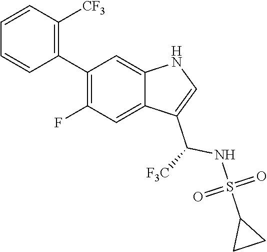 [SMILES: O=S(=O)(N[C@@H](c1c[nH]c2cc(-c3ccccc3C(F)(F)F)c(F)cc12)C(F)(F)F)C1CC1]

A mixture of (1S)-2,2,2-trifluoro-1-[5-fluoro-6-[2-(trifluoromethyl)phenyl]-1H-indol-3-yl]ethanamine (200 mg, 531.53 umol) and cyclopropanesulfonyl chloride (223.54 mg, 1.59 mmol, 161.98 uL) in pyridine (1.5 mL) was stirred at RT overnight. The mixture was acidified to PH=1 with 1N HCl, then was extracted with EtOAc twice. The combined organic phases were washed with brine, dried with Na₂SO₄, filtered and concentrated to get brown oil, which was purified by silica gel column (EA/PE=0-33%) to get N-[(1S)-2,2,2-trifluoro-1-[5-fluoro-6-[2-(trifluoromethyl)phenyl]-1H-indol-3-yl]ethyl]cyclopropanesulfonamide (184 mg, 383.02 umol) as yellow oil.

### Step 5: N-[(1S)-2,2,2-trifluoro-1-[5-fluoro-1-[(3-methyloxetan-3-yl)methyl]-6-[2-(trifluoromethyl)phenyl]indol-3-yl]ethyl]cyclopropanesulfonamide

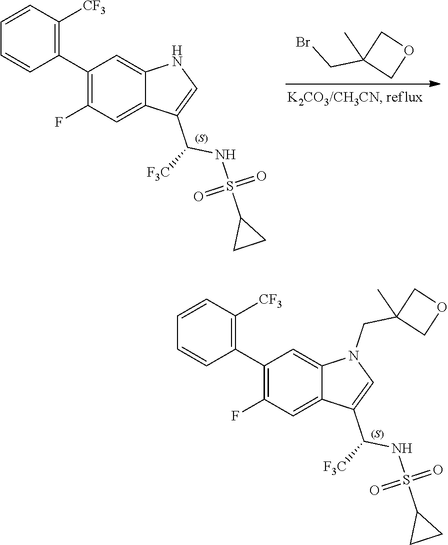

A mixture of N-[(1S)-2,2,2-trifluoro-1-[5-fluoro-6-[2-(trifluoromethyl)phenyl]-1H-indol-3-yl]ethyl]cyclopropanesulfonamide (414 mg, 861.78 umol), 3-(bromomethyl)-3-methyl-oxetane (568.88 mg, 3.45 mmol) and K2CO3 (476.82 mg, 3.45 mmol) in CH3CN (2.5 mL) was stirred at reflux for 4 hours, the mixture was concentrated to remove CH3CN, the residue was poured into water and extracted with EtOAc twice. The combined organic phases were washed with brine, dried with anhydrous Na2SO4 and filtered. The filtrate was concentrated to give the crude product, which was purified prep-HPLC to get N-[(1S)-2,2,2-trifluoro-1-[5-fluoro-1-[(3-methyloxetan-3-yl)methyl]-6-[2-(trifluoromethyl)phenyl]indol-3-yl]ethyl]cyclopropanesulfonamide (32.41 mg, 57.41 umol) as a white solid. ¹H NMR (400 MHz, DMSO) δ 8.58 (s, 1H), 7.86 (d, J=7.7 Hz, 1H), 7.81 (s, 1H), 7.79-7.70 (m, 2H), 7.66 (t, J=7.6 Hz, 1H), 7.59 (d, J=6.1 Hz, 1H), 7.48 (d, J=7.4 Hz, 1H), 5.51 (q, J=7.8 Hz, 1H), 4.42 (ddd, J=18.6, 17.6, 4.9 Hz, 4H), 4.17 (dd, J=9.6, 5.9 Hz, 2H), 2.35-2.24 (m, 1H), 1.17 (s, 3H), 0.88 (d, J=4.3 Hz, 2H), 0.78-0.56 (m, 2H). LCMS: 582.2 m/z [M+H₂O]⁺.

### Example 490: N-[(1S)-2,2,2-trifluoro-1-[5-fluoro-1-(3-hydroxy-2,2-dimethyl-propyl)-6-[2-(trifluoromethyl)phenyl]indol-3-yl]ethyl]cyclopropanesulfonamide (490)

### Step 1: 3-(6-bromo-5-fluoro-indol-1-yl)-2,2-dimethyl-propan-1-ol

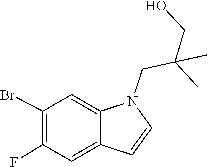 [SMILES: CC(C)(CO)Cn1ccc2cc(F)c(Br)cc21]

To a stirred solution of 6-bromo-5-fluoro-1H-indole (2.0 g, 9.34 mmol), and 1,4,7,10,13,16-hexaoxacyclooctadecane (3.21 g, 12.15 mmol, 2.72 mL) in THF (15 ml) was added sodium;hydride (747.47 mg, 18.69 mmol, 60% purity) at 5° C. Then the mixture was heated to 80° C. To the reaction mixture was added a solution for 5,5-dimethyl-1,3,2-dioxathiane 2,2-dioxide (1.16 g, 7.01 mmol) in THF (5 ml), and the stirring was continued at 80° C. overnight. The reaction mixture was cooled down to 0° C., and conc. HCl was added slowly until pH reaches 1-2. Then the reaction mixture was further stirred at 80° C. for 1 hours. After neutralized with saturated aqueous NaHCO3 solution, the reaction mixture was partitioned between ethyl acetate and water. The organic layer was washed with brine, dried with anhydrous MgSO4, filtered, and evaporated. The residue was purified by column chromatography of silica to give 3-(6-bromo-5-fluoro-indol-1-yl)-2,2-dimethyl-propan-1-ol (1.9 g, 6.33 mmol, 67.74% yield) as a yellow solid.

### Step 2: (S)—N-[(1S)-1-[6-bromo-5-fluoro-1-(3-hydroxy-2,2-dimethyl-propyl)indol-3-yl]-2,2,2-trifluoro-ethyl]-2-methyl-propane-2-sulfinamide

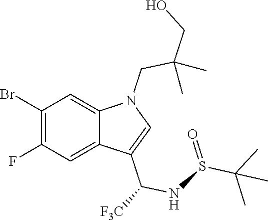

The mixture of 3-(6-bromo-5-fluoro-indol-1-yl)-2,2-dimethyl-propan-1-ol (0.8 g, 2.67 mmol) in DCM (10 mL) was cooled to −20° C., BF₃·Et₂O (1.13 g, 8.00 mmol, 986.78 uL) was added slowly, followed by addition DCM solution of (NE,S)-2-methyl-N-(2,2,2-trifluoroethylidene)propane-2-sulfinamide (1.07 g, 5.33 mmol). The mixture was then continued to stir at −10° C. for 1.5 h and the completion of reaction was monitored by TLC indicating the complete consume of indole. The mixture was diluted with DCM, followed by water. The aqueous layer was extracted with DCM twice. Organic layers were combined and washed with brine and concentrated in vacuo to give (S)—N-[(1S)-1-[6-bromo-5-fluoro-1-(3-hydroxy-2,2-dimethyl-propyl)indol-3-yl]-2,2,2-trifluoro-ethyl]-2-methyl-propane-2-sulfinamide (2.1 g, 4.19 mmol, 157.15% yield). It was used in next step without purification.

### Step 3: 3-[3-[(1S)-1-amino-2,2,2-trifluoro-ethyl]-6-bromo-5-fluoro-indol-1-yl]-2,2-dimethyl-propan-1-ol

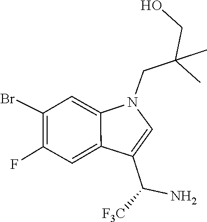 [SMILES: CC(C)(CO)Cn1cc([C@H](N)C(F)(F)F)c2cc(F)c(Br)cc21]

To a (S)—N-[(1S)-1-[6-bromo-5-fluoro-1-(3-hydroxy-2,2-dimethyl-propyl)indol-3-yl]-2,2,2-trifluoro-ethyl]-2-methyl-propane-2-sulfinamide (2.1 g, 4.19 mmol) was added 4N HCl/MeOH (15 mL). The mixture was stirred at RT for 1.5 h. The reaction was concentrated and adjusted pH=8 by addition aq. NaHCO3. The aqueous layer was extracted by DCM (50 mL×2). The combined organic layers were dried over anhydrous Na2SO4, filtered and concentrated. The residue obtained was purified by column chromatography of silica gel eluting with DCM/MeOH from 50/1 to 10/1 to give 3-[3-[(1S)-1-amino-2,2,2-trifluoro-ethyl]-6-bromo-5-fluoro-indol-1-yl]-2,2-dimethyl-propan-1-ol (0.82 g, 2.06 mmol, 49.29% yield) as a brown oil.

### Step 4: 3-[3-[(1S)-1-amino-2,2,2-trifluoro-ethyl]-5-fluoro-6-[2-(trifluoromethyl)phenyl]indol-1-yl]-2,2-dimethyl-propan-1-ol

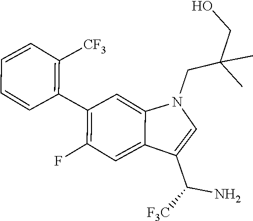 [SMILES: CC(C)(CO)Cn1cc([C@H](N)C(F)(F)F)c2cc(F)c(-c3ccccc3C(F)(F)F)cc21]

A mixture of 3-[3-[(1S)-1-amino-2,2,2-trifluoro-ethyl]-6-bromo-5-fluoro-indol-1-yl]-2,2-dimethyl-propan-1-ol (0.5 g, 1.26 mmol), [2-(trifluoromethyl)phenyl]boronic acid (310.80 mg, 1.64 mmol), cyclopentyl (diphenyl)phosphane;dichloropalladium;iron (276.32 mg, 377.64 umol) and disodium;carbonate (400.26 mg, 3.78 mmol, 158.20 uL) in DIOXANE (5 mL) and H2O (1 mL) was stirred at 80° C. for 2 h under Nitrogen. The mixture was extracted with EA (50 mL×2). The combined organic layers were concentrated, the residue was purified by silica gel column (EA/PE=0-50%) to get 3-[3-[(1S)-1-amino-2,2,2-trifluoro-ethyl]-5-fluoro-6-[2-(trifluoromethyl)phenyl]indol-1-yl]-2,2-dimethyl-propan-1-ol (350 mg, 756.92 umol) as a yellow oil.

### Step 5: N-[(1S)-2,2,2-trifluoro-1-[5-fluoro-1-(3-hydroxy-2,2-dimethyl-propyl)-6-[2-(trifluoromethyl)phenyl]indol-3-yl]ethyl]cyclopropanesulfonamide

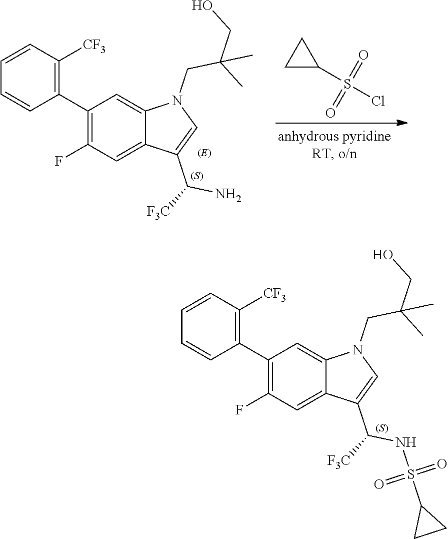

To a mixture of 3-[3-[(1S)-1-amino-2,2,2-trifluoro-ethyl]-5-fluoro-6-[2-(trifluoromethyl)phenyl]indol-1-yl]-2,2-dimethyl-propan-1-ol (200 mg, 432.52 umol) in anhydrous pyridine (5 mL) at RT was added cyclopropanesulfonyl chloride (182.42 mg, 1.30 mmol, 132.19 uL). The mixture was stirred at RT overnight and the completion of reaction was monitored by lcms. The mixture was concentrated and diluted with EtOAc, washed with 2N HCl, water, sat'd NaHCO3 and brine and dried over Na₂SO₄. Solvent was removed in vacuo to obtain the crude which was purified by chromatography on silica gel (EtOAc/hex) to afford N-[(1 S)-2,2,2-trifluoro-1-[5-fluoro-1-(3-hydroxy-2,2-dimethyl-propyl)-6-[2-(trifluoromethyl)phenyl]indol-3-yl]ethyl]cyclopropanesulfonamide (62 mg, 109.44 umol) as a yellow solid. ¹H NMR (400 MHz, MeOD) δ 7.79 (d, J=7.6 Hz, 1H), 7.65 (t, J=7.3 Hz, 1H), 7.62-7.53 (m, 2H), 7.53-7.46 (m, 1H), 7.41 (t, J=7.2 Hz, 2H), 5.40 (dd, J=15.9, 8.0 Hz, 1H), 4.07 (s, 2H), 3.28 (s, 2H), 2.29 (d, J=12.6 Hz, 1H), 1.00 (dd, J=10.6, 7.5 Hz, 2H), 0.89 (d, J=9.6 Hz, 6H), 0.75 (d, J=8.1 Hz, 2H). LCMS: 583.8 m/z [M+18]⁺.

### Example 491: N-[(1S)-1-[1-(2,2-dimethylpropyl)-6-[2-(trifluoromethyl)phenyl]pyrrolo[2,3-b]pyridin-3-yl]-2,2,2-trifluoro-ethyl]cyclopropanesulfonamide (491)

### Step 1: 6-bromo-1-(2,2-dimethylpropyl)pyrrolo[2,3-b]pyridine

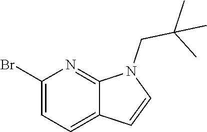

6-bromo-1H-pyrrolo[2,3-b]pyridine (1 g, 5.08 mmol) was added to a suspension of NaH (507.48 mg, 12.69 mmol, 60% purity) in anhydrous DMF (10 mL) in ice-water under nitrogen. The mixture was stirred for 1 h at room temperature, then cooling in ice-water for the addition of 1-bromo-2,2-dimethyl-propane (2.30 g, 15.23 mmol, 1.92 mL) and the resulting mixture was stirred at room temperature overnight under nitrogen. After quenching with water, the aqueous phase was extracted with ethyl acetate twice. The combined organic layer was wash with brine, dried over anhydrous sodium sulfate and filtered. The filtrate was concentrated to give the crude product, which was purified by silica gel column (EA/PE=0-3.3%) to get 6-bromo-1-(2,2-dimethylpropyl)pyrrolo[2,3-b]pyridine (939 mg, 3.51 mmol) as colorless oil.

### Step 2: (S)—N—((S)-1-(6-bromo-1-neopentyl-1H-pyrrolo[2,3-b]pyridin-3-yl)-2,2,2-trifluoroethyl)-2-methylpropane-2-sulfinamide

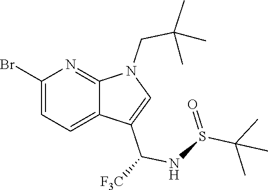

The mixture of 6-bromo-1-(2,2-dimethylpropyl)pyrrolo[2,3-b]pyridine (939 mg, 3.51 mmol) in anhydrous CH₂Cl₂ (10 mL) was cooled to −10° C., BF3·OEt2 (997.68 mg, 7.03 mmol, 867.55 uL) was added slowly, followed by addition of a solution of (S,E)-2-methyl-N-(2,2,2-trifluoroethylidene)propane-2-sulfinamide in DCM. The mixture was heated to reflux overnight under Nitrogen. The mixture was diluted with DCM, followed by water. The aqueous layer was extracted with DCM twice. Organic layers were combined and washed with brine and concentrated in vacuo to obtain the crude as brown oil, which was used directly next step.

### Step 3: (1S)-1-[6-bromo-1-(2,2-dimethylpropyl)pyrrolo[2,3-b]pyridin-3-yl]-2,2,2-trifluoro-ethanamine

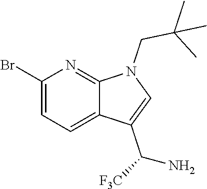 [SMILES: CC(C)(C)Cn1cc([C@H](N)C(F)(F)F)c2ccc(Br)nc21]

A mixture of (S)—N-[(1S)-1-[6-bromo-1-(2,2-dimethylpropyl)pyrrolo[2,3-b]pyridin-3-yl]-2,2,2-trifluoro-ethyl]-2-methyl-propane-2-sulfinamide (1.64 g, 3.51 mmol) in 4M HCl/MeOH (25 mL) was stirred at RT for 2 hours, the mixture was concentrated, the residue was basified to pH=8 with sat. NaHCO3, the aqueous phase was extracted with EtOAc twice, the combined organic phases were washed with brine and concentrated to get crude product, which was purified by silica gel column (EA/PE=0-50%) to get (1S)-1-[6-bromo-1-(2,2-dimethylpropyl)pyrrolo[2,3-b]pyridin-3-yl]-2,2,2-trifluoro-ethanamine (867 mg, 2.38 mmol) as light yellow oil.

### Step 4: (1S)-1-[1-(2,2-dimethylpropyl)-6-[2-(trifluoromethyl)phenyl]pyrrolo[2,3-b]pyridin-3-yl]-2,2,2-trifluoro-ethanamine

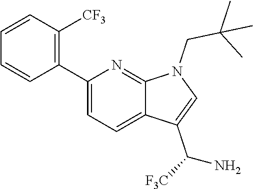 [SMILES: CC(C)(C)Cn1cc([C@H](N)C(F)(F)F)c2ccc(-c3ccccc3C(F)(F)F)nc21]

A mixture of (1S)-1-[6-bromo-1-(2,2-dimethylpropyl)pyrrolo[2,3-b]pyridin-3-yl]-2,2,2-trifluoro-ethanamine (400 mg, 1.10 mmol), [2-(trifluoromethyl)phenyl]boronic acid (312.89 mg, 1.65 mmol) and K2CO3 (455.37 mg, 3.29 mmol) in dioxane (8 mL) and water (1.6 mL) was stirred at 80° C. overnight under Nitrogen. The mixture was added water and EtOAc, the mixture was stirred and separated, the aqueous phase was extracted with EtOAc twice, the combined organic phases were washed with brine and dried with Na2SO4, filtered through silica gel to get oil, which was purified by silica gel column (EA/PE=0-50%) to get (1S)-1-[1-(2,2-dimethylpropyl)-6-[2-(trifluoromethyl)phenyl]pyrrolo[2,3-b]pyridin-3-yl]-2,2,2-trifluoro-ethanamine (420 mg, 978.11 μmol, 89.06% yield) as brown oil.

### Step 5: N-[(1S)-1-[1-(2,2-dimethylpropyl)-6-[2-(trifluoromethyl)phenyl]pyrrolo[2,3-b]pyridin-3-yl]-2,2,2-trifluoro-ethyl]cyclopropanesulfonamide

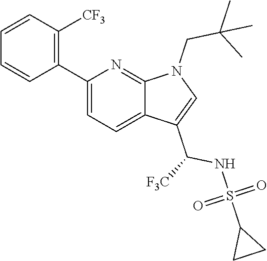 [SMILES: CC(C)(C)Cn1cc([C@H](NS(=O)(=O)C2CC2)C(F)(F)F)c2ccc(-c3ccccc3C(F)(F)F)nc21]

A mixture of (1S)-1-[1-(2,2-dimethylpropyl)-6-[2-(trifluoromethyl)phenyl]pyrrolo[2,3-b]pyridin-3-yl]-2,2,2-trifluoro-ethanamine (153 mg, 356.31 μmol) and cyclopropanesulfonyl chloride (150.28 mg, 1.07 mmol, 108.90 uL) in pyridine (1.5 mL) was stirred at RT overnight. The mixture was concentrated to remove pyridine to get black oil, which was purified by silica gel column (EA/PE=0-50%) to get crude product as brown oil, which was purified by prep-HPLC twice to get N-[(1S)-1-[1-(2,2-dimethylpropyl)-6-[2-(trifluoromethyl)phenyl]pyrrolo[2,3-b]pyridin-3-yl]-2,2,2-trifluoro-ethyl]cyclopropanesulfonamide (56.40 mg, 105.71 μmol) as a white solid. ¹H NMR (400 MHz, DMSO) δ 8.72 (s, 1H), 8.38 (d, J=8.1 Hz, 1H), 7.95-7.85 (m, 2H), 7.78 (t, J=7.3 Hz, 1H), 7.71-7.66 (m, 1H), 7.59 (d, J=7.5 Hz, 1H), 7.29 (d, J=8.2 Hz, 1H), 5.60 (d, J=7.9 Hz, 1H), 4.13 (s, 2H), 2.42-2.33 (m, 1H), 0.97-0.89 (m, 11H), 0.76 (ddd, J=8.8, 5.4, 2.6 Hz, 1H), 0.68 (ddd, J=8.6, 5.4, 2.2 Hz, 1H). LCMS: 533.8 m/z [M+H]⁺.

### Example 492: N-[(1S)-1-[1-(2,2-dimethylpropyl)-6-[2-(trifluoromethyl)phenyl]pyrrolo[2,3-b]pyridin-3-yl]-2,2,2-trifluoro-ethyl]cyclobutanesulfonamide (492)

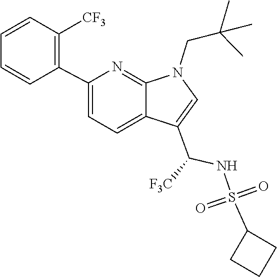 [SMILES: CC(C)(C)Cn1cc([C@H](NS(=O)(=O)C2CCC2)C(F)(F)F)c2ccc(-c3ccccc3C(F)(F)F)nc21]

A mixture of (1S)-1-[1-(2,2-dimethylpropyl)-6-[2-(trifluoromethyl)phenyl]pyrrolo[2,3-b]pyridin-3-yl]-2,2,2-trifluoro-ethanamine (270 mg, 628.78 μmol) and cyclobutanesulfonyl chloride (500 mg, 3.23 mmol) in anhydrous pyridine (1 mL) was stirred at 100° C. for 3.5 hours in the microwave. The mixture was concentrated for remove pyridine to get black oil, which was purified by silica gel column (EA/PE=0-50%) to get crude product as brown oil, which was purified by prep-HPLC to get N-[(1S)-1-[1-(2,2-dimethylpropyl)-6-[2-(trifluoromethyl)phenyl]pyrrolo[2,3-b]pyridin-3-yl]-2,2,2-trifluoro-ethyl]cyclobutanesulfonamide (27.52 mg, 50.26 μmol, 7.99% yield) as a white solid. ¹H NMR (400 MHz, DMSO) δ 8.69 (s, 1H), 8.37 (d, J=8.1 Hz, 1H), 7.92-7.86 (m, 2H), 7.78 (t, J=7.5 Hz, 1H), 7.68 (t, J=7.6 Hz, 1H), 7.59 (d, J=7.6 Hz, 1H), 7.28 (d, J=8.2 Hz, 1H), 5.56 (d, J=6.9 Hz, 1H), 4.13 (s, 2H), 3.78-3.68 (m, 1H), 2.31-2.15 (m, 2H), 2.03-1.85 (m, 2H), 1.81-1.65 (m, 2H), 0.93 (s, 9H). LCMS: 548.2 m/z [M+H]⁺.

### Example 493: 3-(2-(cyclopropylsulfonyl)ethyl)-1-neopentyl-6-(2-(trifluoromethyl)phenyl)-1H-indole (493)

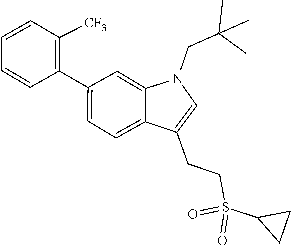 [SMILES: CC(C)(C)Cn1cc(CCS(=O)(=O)C2CC2)c2ccc(-c3ccccc3C(F)(F)F)cc21]

The title compound was prepared following the same general protocol as described for Step 5, Example 1, using (2-(trifluoromethyl)phenyl)boronic acid and 6-bromo-3-(2-(cyclopropylsulfonyl)ethyl)-1-neopentyl-1H-indole. ESI-MS (m/z): 464.2 [M+1]⁺.

### Example 494: (S)—N-(2,2,2-trifluoro-1-(1-neopentyl-6-(2-(trifluoromethyl)phenyl)-1H-indol-3-yl)ethyl)cyclobutanesulfonamide (494)

### Step 1: (S)-2,2,2-trifluoro-1-(1-neopentyl-6-(2-(trifluoromethyl)phenyl)-1H-pyrrolo[2,3-b]pyridin-3-yl)ethanamine

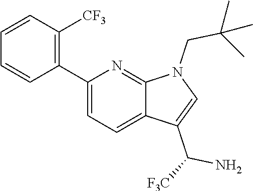 [SMILES: CC(C)(C)Cn1cc([C@H](N)C(F)(F)F)c2ccc(-c3ccccc3C(F)(F)F)nc21]

A mixture of rac-(1S)-1-[6-bromo-1-(2,2-dimethylpropyl)indol-3-yl]-2,2,2-trifluoro-ethanamine (400 mg, 1.10 mmol), [2-(trifluoromethyl)phenyl]boronic acid (250.99 mg, 1.32 mmol), Pd(dppf)Cl2 (80.58 mg, 110.13 μmol) and Na2CO3 (291.81 mg, 2.75 mmol) in dioxane (8 mL) and water (2 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-50%) to get rac-(1S)-1-[1-(2,2-dimethylpropyl)-6-[2-(trifluoromethyl)phenyl]indol-3-yl]-2,2,2-trifluoro-ethanamine (405 mg, 945.35 μmol, 85.84% yield) as yellow oil.

### Step 2: (S)—N-(2,2,2-trifluoro-1-(1-neopentyl-6-(2-(trifluoromethyl)phenyl)-1H-indol-3-yl)ethyl)cyclobutanesulfonamide

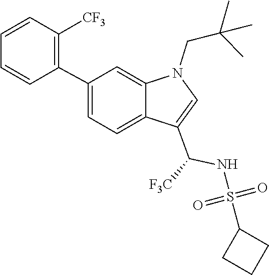 [SMILES: CC(C)(C)Cn1cc([C@H](NS(=O)(=O)C2CCC2)C(F)(F)F)c2ccc(-c3ccccc3C(F)(F)F)cc21]

A mixture of (S)-2,2,2-trifluoro-1-(1-neopentyl-6-(2-(trifluoromethyl)phenyl)-1H-pyrrolo[2,3-b]pyridin-3-yl)ethanamine (405 mg, 945.35 μmol) and cyclobutanesulfonyl chloride (263.10 mg, 1.70 mmol) in anhydrous pyridine (0.8 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(2,2,2-trifluoro-1-(1-neopentyl-6-(2-(trifluoromethyl)phenyl)-1H-indol-3-yl)ethyl)cyclobutanesulfonamide (141.76 mg, 259.36 μmol, 27.44% yield) as a white solid. ¹H NMR (400 MHz, DMSO) δ 8.58 (s, 1H), 7.81 (dd, J=15.2, 7.8 Hz, 2H), 7.72 (t, J=7.3 Hz, 1H), 7.61 (dd, J=16.1, 8.4 Hz, 2H), 7.51 (s, 1H), 7.43 (d, J=7.5 Hz, 1H), 7.00 (d, J=8.3 Hz, 1H), 5.42 (d, J=7.4 Hz, 1H), 4.01 (s, 2H), 3.71-3.59 (m, 1H), 2.26-2.11 (m, 2H), 1.99-1.83 (m, 2H), 1.70 (dt, J=9.0, 6.8 Hz, 2H), 0.92 (s, 9H). LCMS: 546.7 m/z [M+1]⁺.

### Example 495: (S)—N-(1-(6-(2-cyanophenyl)-1-neopentyl-1H-indol-3-yl)-2,2,2-trifluoroethyl)cyclobutanesulfonamide (495)

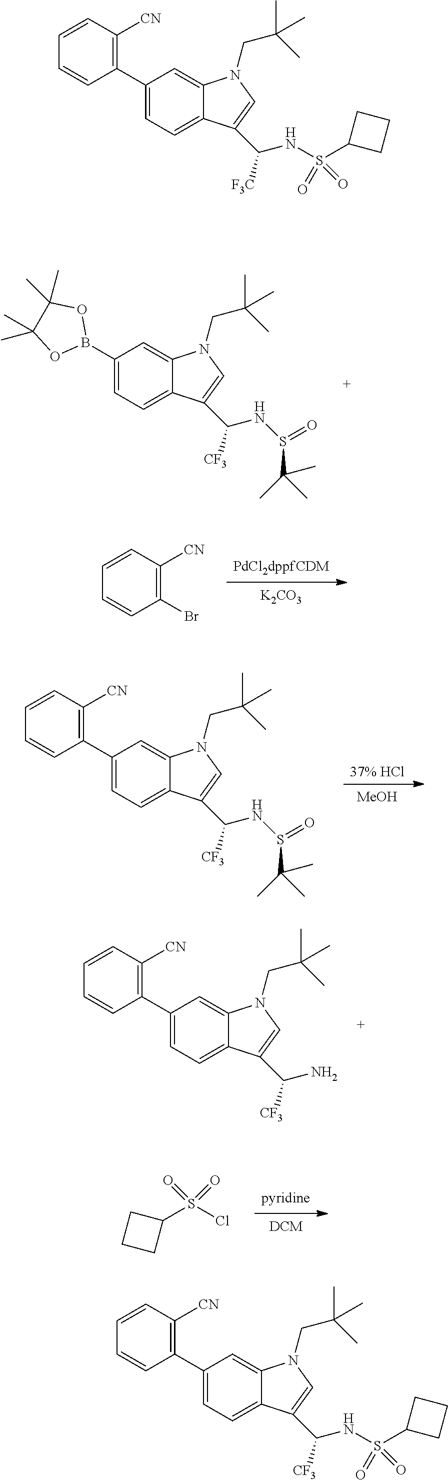

### Step 1: (S)—N—((S)-1-(6-(2-cyanophenyl)-1-neopentyl-1H-indol-3-yl)-2,2,2-trifluoroethyl)-2-methylpropane-2-sulfinamide

The title compound was prepared following the same general protocol as described for Step 5, Example 1, using (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide and 2-bromobenzonitrile. ESI-MS (m/z): 490.2 [M+1]⁺.

### Step 2: (S)-2-(3-(1-amino-2,2,2-trifluoroethyl)-1-neopentyl-1H-indol-6-yl)benzonitrile

The title compound was prepared following the same general protocol as described for Step 4, Example 116, using (S)—N—((S)-1-(6-(2-cyanophenyl)-1-neopentyl-1H-indol-3-yl)-2,2,2-trifluoroethyl)-2-methylpropane-2-sulfinamide. ESI-MS (m/z): 369.2 [M-NH₂]⁺.

### Step 3: (S)—N-(1-(6-(2-cyanophenyl)-1-neopentyl-1H-indol-3-yl)-2,2,2-trifluoroethyl)cyclobutanesulfonamide

The title compound was prepared following the same general protocol as described for Step 5, Example 116, (1S)-1-(1-(sec-butyl)-6-(2-(trifluoromethyl)phenyl)-1H-indol-3-yl)-2,2,2-trifluoroethan-1-amine and cyclobutanesulfonyl chloride. ESI-MS (m/z): 504.2 [M+1]⁺.

### Example 496: (S)—N-(2,2,2-trifluoro-1-(6-(2-fluoro-6-(trifluoromethyl)phenyl)-1-neopentyl-1H-indol-3-yl)ethyl)cyclobutanesulfonamide (496)

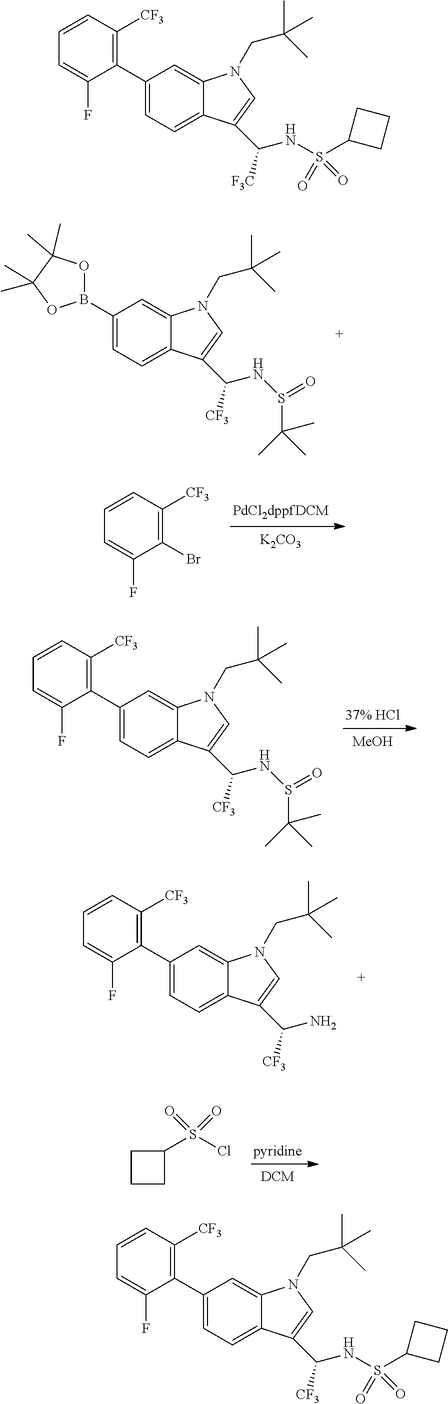

### Step 1: (S)-2-methyl-N—((S)-2,2,2-trifluoro-1-(6-(2-fluoro-6-(trifluoromethyl)phenyl)-1-neopentyl-1H-indol-3-yl)ethyl)propane-2-sulfinamide

The title compound was prepared following the same general protocol as described for Step 5, Example 1, using (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide and 2-bromo-1-fluoro-3-(trifluoromethyl)benzene. ESI-MS (m/z): 551.2 [M+1]⁺.

### Step 2: (S)-2,2,2-trifluoro-1-(6-(2-fluoro-6-(trifluoromethyl)phenyl)-1-neopentyl-1H-indol-3-yl)ethan-1-amine

The title compound was prepared following the same general protocol as described for Step 4, Example 116, (S)-2-methyl-N—((S)-2,2,2-trifluoro-1-(6-(2-fluoro-6-(trifluoromethyl)phenyl)-1-neopentyl-1H-indol-3-yl)ethyl)propane-2-sulfinamide. ESI-MS (m/z): 430.2 [M-NH₂]⁺.

### Step 3: (S)—N-(2,2,2-trifluoro-1-(6-(2-fluoro-6-(trifluoromethyl)phenyl)-1-neopentyl-1H-indol-3-yl)ethyl)cyclobutanesulfonamide

The title compound was prepared following the same general protocol as described for Step 5, Example 116, using (S)-2,2,2-trifluoro-1-(6-(2-fluoro-6-(trifluoromethyl)phenyl)-1-neopentyl-1H-indol-3-yl)ethan-1-amine and cyclobutanesulfonyl chloride. ESI-MS (m/z): 565.2 [M+1]⁺.

### Example 497: (S)—N-(2,2,2-trifluoro-1-(6-(4-fluoro-2-(trifluoromethyl)phenyl)-1-neopentyl-1H-indol-3-yl)ethyl)cyclobutanesulfonamide (497)

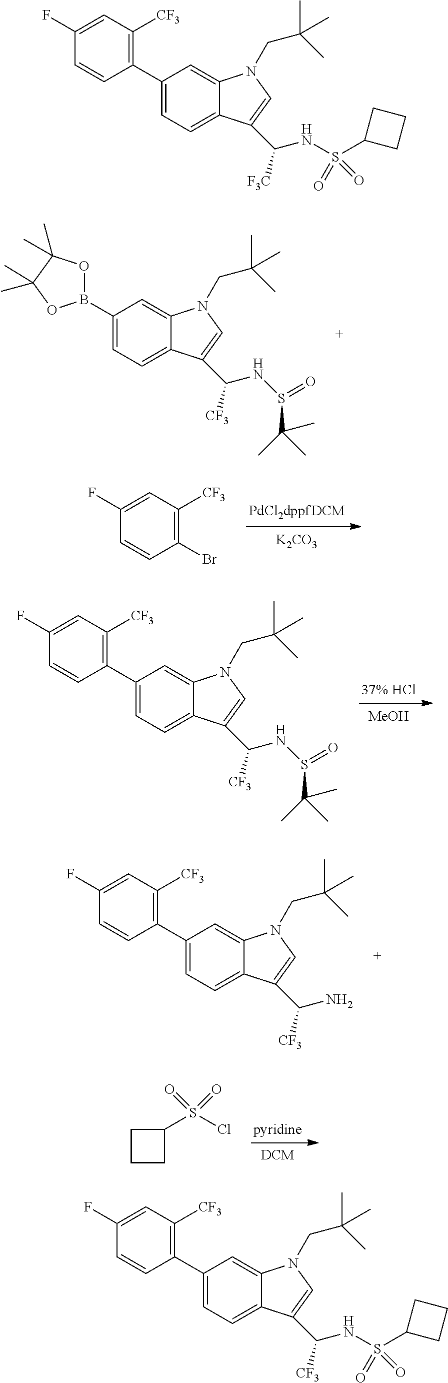

### Step 1: (S)-2-methyl-N—((S)-2,2,2-trifluoro-1-(6-(4-fluoro-2-(trifluoromethyl)phenyl)-1-neopentyl-1H-indol-3-yl)ethyl)propane-2-sulfinamide

The title compound was prepared following the same general protocol as described for Step 5, Example 1, using (S)-2-methyl-N—((S)-2,2,2-trifluoro-1-(1-neopentyl-6-(4,4,5,5-tetramethyl-1,3,2-dioxaborolan-2-yl)-1H-indol-3-yl)ethyl)propane-2-sulfinamide and 1-bromo-4-fluoro-2-(trifluoromethyl)benzene. ESI-MS (m/z): 551.2 [M+1]⁺.

### Step 2: (S)-2,2,2-trifluoro-1-(6-(4-fluoro-2-(trifluoromethyl)phenyl)-1-neopentyl-1H-indol-3-yl)ethan-1-amine

The title compound was prepared following the same general protocol as described for Step 4, Example 116, (S)-2-methyl-N—((S)-2,2,2-trifluoro-1-(6-(2-fluoro-6-(trifluoromethyl)phenyl)-1-neopentyl-1H-indol-3-yl)ethyl)propane-2-sulfinamide. ESI-MS (m/z): 430.2 [M-NH₂]⁺.

### Step 3: (S)—N-(2,2,2-trifluoro-1-(6-(4-fluoro-2-(trifluoromethyl)phenyl)-1-neopentyl-1H-indol-3-yl)ethyl)cyclobutanesulfonamide

The title compound was prepared following the same general protocol as described for Step 5, Example 116, using (S)-2,2,2-trifluoro-1-(6-(2-fluoro-6-(trifluoromethyl)phenyl)-1-neopentyl-1H-indol-3-yl)ethan-1-amine and cyclobutanesulfonyl chloride. ESI-MS (m/z): 565.2 [M+1]⁺.

### Example 498: 3-(3-(cyclopropylsulfonyl)-1,1,1-trifluoropropan-2-yl)-1-isobutyl-6-(2-(trifluoromethyl)phenyl)-1H-indole (498)

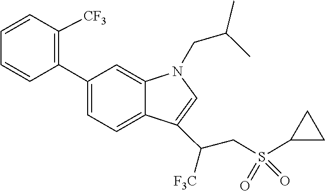 [SMILES: CC(C)Cn1cc(C(CS(=O)(=O)C2CC2)C(F)(F)F)c2ccc(-c3ccccc3C(F)(F)F)cc21]

### Step1: diisopropyl ((cyclopropylsulfonyl)methyl)phosphonate

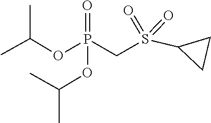 [SMILES: CC(C)OP(=O)(CS(=O)(=O)C1CC1)OC(C)C]

A mixture of diisopropyl (bromomethyl)phosphonate (0.5 g, 1.93 mmol), sodium cyclopropanesulfinate (0.37 g, 2.895 mmol) and TBAI (0.071 g, 0.19 mmol) in DMF (4 mL) was heated overnight in a 100° C. oil bath. The mixture was cooled to rt and diluted with EtOAc and water. The layers were separated, and the organic layer was washed with saturated NaHCO₃, brine, dried (Na₂SO₄) and filtered. The solvent was removed and the crude was purified by silica gel to obtain the title compound. ¹HNMR (CDCl₃, 400 MHz) 4.87-4.79 (m, 2H), 3.56 (d, J=16.0 Hz, 2H), 3.02-2.95 (m, 1H), 1.35 (dd, J=8.0 Hz, 4.0 Hz, 12H), 1.30-1.25 (m, 2H), 1.08-1.04 (m, 2H)

### Step 2: 3-(1-(cyclopropylsulfonyl)-3,3,3-trifluoroprop-1-en-2-yl)-1-isobutyl-6-(2-(trifluoromethyl)phenyl)-1H-indole

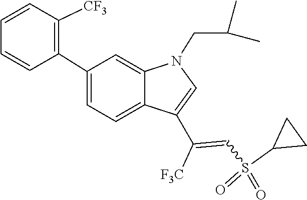 [SMILES: CC(C)Cn1cc(C(=CS(=O)(=O)C2CC2)C(F)(F)F)c2ccc(-c3ccccc3C(F)(F)F)cc21]

To the mixture of diisopropyl ((cyclopropylsulfonyl)methyl)phosphonate (0.104 g, 0.367 mmol) in THF (1.5 mL) at −78° C. under argon was added dropwise LiHMDS (0.73 mL, 1M in THF, 0.73 mmol). After 30 min, 2,2,2-trifluoro-1-(1-isobutyl-6-(2-(trifluoromethyl)phenyl)-1H-indol-3-yl)ethan-1-one (0.151 g, 0.367 mmol) was added. The reaction was allowed to warm to rt overnight. Saturated NH₄Cl was added and extracted with EtOAc. The solvent was removed and the crude was purified by silica gel to obtain the title compound. ¹HNMR (CDCl₃, 400 MHz) 7.76 (d, J=8.0 Hz, 1H), 7.64 (d, J=8.0 Hz, 1H), 7.56 (s, 1H), 7.46 (t, J=8.0 Hz, 2H), 7.41 (d, J=8.0 Hz, 1H), 7.32 (s, 1H), 7.16-7.14 (m, 2H), 3.95 (d, J=8.0 Hz, 2H), 2.36-2.20 (m, 2H), 1.16-1.11 (m, 2H), 0.96 (d, J=8.0 Hz, 6H), 0.97-0.79 (m, 2H)

### Step3: 3-(3-(cyclopropylsulfonyl)-1,1,1-trifluoropropan-2-yl)-1-isobutyl-6-(2-(trifluoromethyl)phenyl)-1H-indole

To a solution of 3-(1-(cyclopropylsulfonyl)-3,3,3-trifluoroprop-1-en-2-yl)-1-isobutyl-6-(2-(trifluoromethyl)phenyl)-1H-indole (0.007 g) in EtOH was added Pd/C (0.003 g). The mixture was stirred under a H₂ balloon overnight at rt. The mixture was filtered and purified by prep. HPLC to obtain the title compound. HNMR (CDCl₃, 400 MHz) 7.76 (d, J=8.0 Hz, 1H), 7.68 (d, J=8.0 Hz, 1H), 7.56 (t, J=8.0 Hz, 2H), 7.46 (t, J=8.0 Hz, 2H), 7.41 (d, J=8.0 Hz, 1H), 7.29 (s, 1H), 7.22 (s, 1H), 7.13 (d, J=8.0 Hz, 1H), 4.38-4.32 (m, 1H), 3.94-3.88 (m, 2H), 3.65 (d, J=8.0 Hz, 1H), 2.21-2.13 (m, 1H), 1.61-1.51 (m, 1H), 1.29-1.10 (m, 1H), 0.82-0.98 (m, 7H), 0.64-0.58 (m, 1H), 0.41-0.33 (m, 1H)

### Example 499: (S)—N-(2,2,2-trifluoro-1-(1-isobutyl-6-(2-(trifluoromethyl)phenyl)-1H-indol-3-yl)ethyl)cyclobutanesulfonamide (499)

### Step 1: (S)-1-(6-bromo-1-isobutyl-1H-indol-3-yl)-2,2,2-trifluoroethanamine hydrochloride

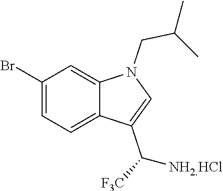

A mixture of rac-(S)-2-methyl-N-[rac-(1S)-1-(6-bromo-1-isobutyl-indol-3-yl)-2,2,2-trifluoro-ethyl]propane-2-sulfinamide (450 mg, 992.59 μmol) in 4M HCl(g)/ethanol (8 mL) was stirred for 2 h, the mixture was concentrated to rac-(1S)-1-(6-bromo-1-isobutyl-indol-3-yl)-2,2,2-trifluoro-ethanamine;hydrochloride (382.79 mg, 992.58 μmol, 100.00% yield) as a yellow solid, which was used directly next step.

### Step 2: (S)-2,2,2-trifluoro-1-(1-isobutyl-6-(2-(trifluoromethyl)phenyl)-1H-indol-3-yl)ethanamine

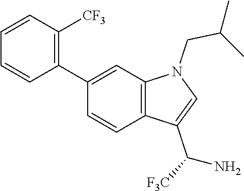 [SMILES: CC(C)Cn1cc([C@H](N)C(F)(F)F)c2ccc(-c3ccccc3C(F)(F)F)cc21]

A mixture of rac-(1S)-1-(6-bromo-1-isobutyl-indol-3-yl)-2,2,2-trifluoro-ethanamine;hydrochloride (382.79 mg, 992.58 μmol), [2-(trifluoromethyl)phenyl]boronic acid (226.22 mg, 1.19 mmol), Pd(dppf)Cl2 (72.63 mg, 99.26 μmol) and Na2CO3 (736.42 mg, 6.95 mmol) in dioxane (8 mL) and water (2 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-50%) to get rac-(1S)-2,2,2-trifluoro-1-[1-isobutyl-6-[2-(trifluoromethyl)phenyl]indol-3-yl]ethanamine (385 mg, 929.08 μmol, 93.60% yield) as yellow oil.

### Step 3: (S)—N-(2,2,2-trifluoro-1-(1-isobutyl-6-(2-(trifluoromethyl)phenyl)-1H-indol-3-yl)ethyl)cyclobutanesulfonamide

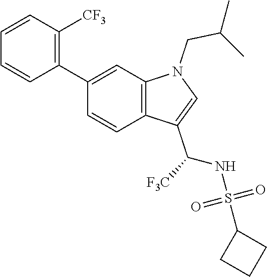 [SMILES: CC(C)Cn1cc([C@H](NS(=O)(=O)C2CCC2)C(F)(F)F)c2ccc(-c3ccccc3C(F)(F)F)cc21]

A mixture of (S)-2,2,2-trifluoro-1-(1-isobutyl-6-(2-(trifluoromethyl)phenyl)-1H-indol-3-yl)ethanamine (305 mg, 736.03 μmol) and cyclobutanesulfonyl chloride (182.08 mg, 1.18 mmol) in anhydrous pyridine (0.8 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(2,2,2-trifluoro-1-(1-isobutyl-6-(2-(trifluoromethyl)phenyl)-1H-indol-3-yl)ethyl)cyclobutanesulfonamide (122.48 mg, 229.99 μmol, 31.25% yield) as a white solid. ¹H NMR (400 MHz, DMSO) δ 8.55 (s, 1H), 7.82 (t, J=8.8 Hz, 2H), 7.72 (t, J=7.4 Hz, 1H), 7.66 (s, 1H), 7.61 (t, J=7.7 Hz, 1H), 7.48 (d, J=5.1 Hz, 2H), 7.04 (d, J=8.3 Hz, 1H), 5.42 (d, J=6.4 Hz, 1H), 4.02 (d, J=7.2 Hz, 2H), 3.76-3.59 (m, 1H), 2.21 (ddd, J=21.5, 13.1, 5.9 Hz, 2H), 2.08 (dt, J=13.5, 6.8 Hz, 1H), 2.02-1.85 (m, 2H), 1.80-1.65 (m, 2H), 0.85 (t, J=7.0 Hz, 6H). LCMS: 532.7 m/z [M+1]⁺.

### Example 500: (S)—N-(2,2,2-trifluoro-1-(6-methyl-1-neopentyl-1H-indol-3-yl)ethyl)cyclobutanesulfonamide (500)

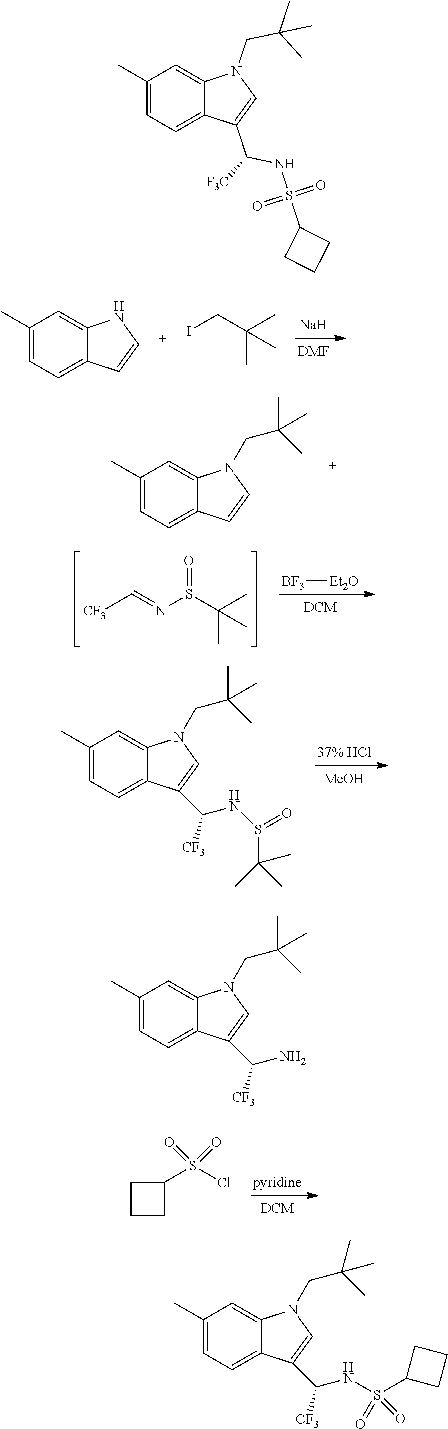

### Step 1: 6-methyl-1-neopentyl-1H-indole

The title compound was prepared following the same general protocol as described for Step 1, Example 1, using 6-methyl-1H-indole and 1-iodo-2,2-dimethylpropane. ESI-MS (m/z): 202.1 [M+1]⁺.

### Step 2. (S)-2-methyl-N—((S)-2,2,2-trifluoro-1-(6-methyl-1-neopentyl-1H-indol-3-yl)ethyl)propane-2-sulfinamide

The title compound was prepared following the same general protocol as described for Step 3, Example 116, using 6-methyl-1-neopentyl-1H-indole. ESI-MS (m/z): 403.2 [M+1]⁺.

### Step 3: (S)-2,2,2-trifluoro-1-(6-methyl-1-neopentyl-1H-indol-3-yl)ethan-1-amine

The title compound was prepared following the same general protocol as described for Step 4, Example 116, using (S)-2-methyl-N—((S)-2,2,2-trifluoro-1-(6-methyl-1-neopentyl-1H-indol-3-yl)ethyl)propane-2-sulfinamide. ESI-MS (m/z): 336.1 [M-NH₂]⁺.

### Step 4: (S)—N-(2,2,2-trifluoro-1-(6-methyl-1-neopentyl-1H-indol-3-yl)ethyl)cyclobutanesulfonamide

The title compound was prepared following the same general protocol as described for Step 5, Example 116, using (S)-2,2,2-trifluoro-1-(6-methyl-1-neopentyl-1H-indol-3-yl)ethan-1-amine and cyclobutanesulfonyl chloride. ESI-MS (m/z): 417.2 [M+1]⁺.

### Example 501: (S)—N-(1-(6-cyclopropyl-1-neopentyl-1H-indol-3-yl)-2,2,2-trifluoroethyl)cyclobutanesulfonamide (501)

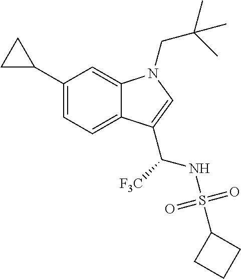 [SMILES: CC(C)(C)Cn1cc([C@H](NS(=O)(=O)C2CCC2)C(F)(F)F)c2ccc(C3CC3)cc21]

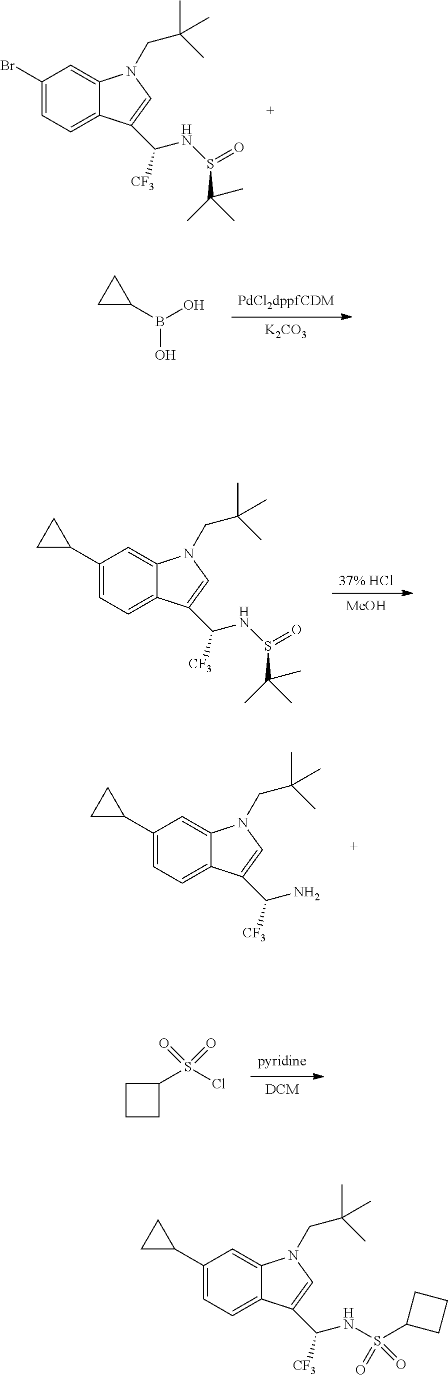

### Step 1. (S)—N—((S)-1-(6-cyclopropyl-1-neopentyl-1H-indol-3-yl)-2,2,2-trifluoroethyl)-2-methylpropane-2-sulfinamide

The title compound was prepared following the same general protocol as described for Step 5, Example 1, using (S)—N—((S)-1-(6-bromo-1-neopentyl-1H-indol-3-yl)-2,2,2-trifluoroethyl)-2-methylpropane-2-sulfinamide and cyclopropylboronic acid. ESI-MS (m/z): 429.2 [M+1]⁺.

### Step 2: (S)-1-(6-cyclopropyl-1-neopentyl-1H-indol-3-yl)-2,2,2-trifluoroethan-1-amine

The title compound was prepared following the same general protocol as described for Step 4, Example 116, using (S)—N—((S)-1-(6-cyclopropyl-1-neopentyl-1H-indol-3-yl)-2,2,2-trifluoroethyl)-2-methylpropane-2-sulfinamide. ESI-MS (m/z): 308.1 [M-NH₂]⁺.

### Step 3: (S)—N-(1-(6-cyclopropyl-1-neopentyl-1H-indol-3-yl)-2,2,2-trifluoroethyl)cyclobutanesulfonamide

The title compound was prepared following the same general protocol as described for Step 5, Example 116, using (S)-2,2,2-trifluoro-1-(6-methyl-1-neopentyl-1H-indol-3-yl)ethan-1-amine and cyclobutanesulfonyl chloride. ESI-MS (m/z): 443.2 [M+1]⁺.

### Example 502: N-(2-(5-(2,4-bis(trifluoromethyl)phenyl)-3-neopentyl-1H-indol-1-yl)ethyl)ethanesulfonamide (502)

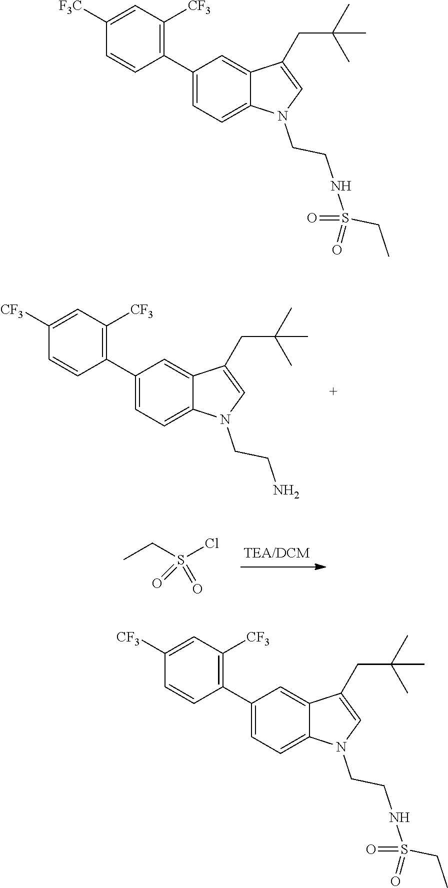

The title compound was prepared following the same general protocol as described for Step 5, Example 116, using 2-(5-(2,4-bis(trifluoromethyl)phenyl)-3-neopentyl-1H-indol-1-yl)ethan-1-amine and ethanesulfonyl chloride. ESI-MS (m/z): 535.2 [M+1]⁺.

### Example 503: N-(2-(5-(2,4-bis(trifluoromethyl)phenyl)-3-neopentyl-1H-indol-1-yl)ethyl)cyclopentanesulfonamide (503)

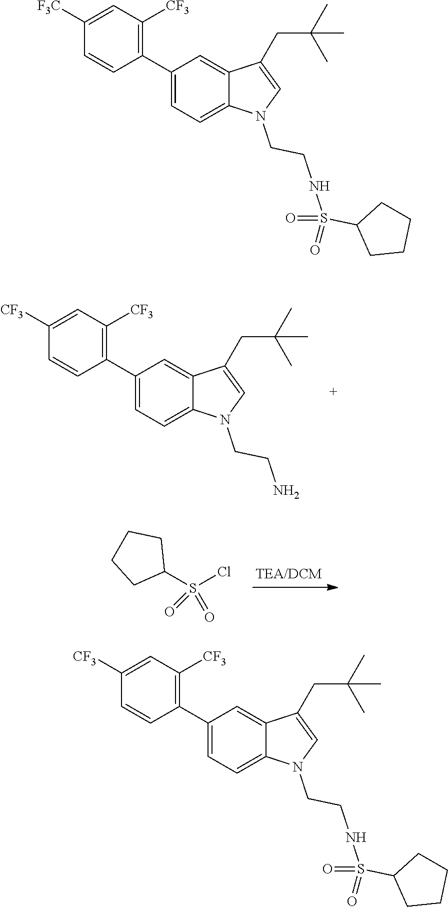

The title compound was prepared following the same general protocol as described for Step 5, Example 116, using 2-(5-(2,4-bis(trifluoromethyl)phenyl)-3-neopentyl-1H-indol-1-yl)ethan-1-amine and cyclopentanesulfonyl chloride. ESI-MS (m/z): 575.2 [M+1]⁺.

### Example 504: (S)—N-(2,2,2-trifluoro-1-(1-isobutyryl-6-(2-(trifluoromethyl)phenyl)-1H-indol-3-yl)ethyl)cyclobutanesulfonamide (504)

### Step 1: 1-(6-bromoindol-1-yl)-2-methyl-propan-1-one

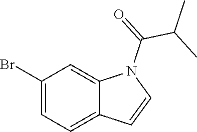

To a solution of 6-bromo-1H-indole (1.2 g, 6.12 mmol) and Triethylamine (929.09 mg, 9.18 mmol, 1.28 mL) in anhydrous CH2Cl2 (25 mL) was added 2-methylpropanoyl chloride (782.65 mg, 7.35 mmol, 769.56 μL) dropwise at room temperature. After stirring for overnight at room temperature, the solvent was removed under reduced pressure and the residue was partitioned between EtOAc and brine. The organic layer was collected, and the aqueous layer was extracted by EtOAc for two times. The combined organic layers were dried over Na2SO4, filtered, and concentrated under reduced pressure. The residue was purified by chromatography on silica gel (Petroleum ether/EtOAc) to give 1-(6-bromoindol-1-yl)-2-methyl-propan-1-one (1.55 g, 5.82 mmol, 95.15% yield) as yellow oil.

### Step 2: (S)—N—((S)-1-(6-bromo-1-isobutyryl-1H-indol-3-yl)-2,2,2-trifluoroethyl)-2-methylpropane-2-sulfinamide

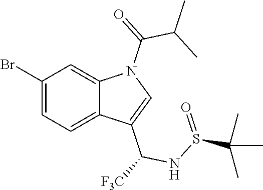 [SMILES: CC(C)C(=O)n1cc([C@H](N[S@@](=O)C(C)(C)C)C(F)(F)F)c2ccc(Br)cc21]

To a stirred solution of 1-(6-bromoindol-1-yl)-2-methyl-propan-1-one (1.3 g, 4.88 mmol) in anhydrous CH2Cl2 (7 mL) was added BF3·OEt2 (2.77 g, 19.54 mmol, 2.41 mL) slowly at −10° C. under Nitrogen, followed by addition of a solution of (S,E)-2-methyl-N-(2,2,2-trifluoroethylidene)propane-2-sulfinamide in DCM. The resulting mixture was raised to reflux overnight. Then the mixture was cooled and added water. The mixture was separated organic phase, the aqueous phase was extracted with CH2Cl2, the combined organic phases were washed with brine, dried with anhydrous Na2SO4 and filtered. The filtrate was concentrated to get (S)—N—((S)-1-(6-bromo-1-isobutyryl-1H-indol-3-yl)-2,2,2-trifluoroethyl)-2-methylpropane-2-sulfinamide as yellow oil, which was directly next step without purification.

### Step 3: (S)-1-(3-(1-amino-2,2,2-trifluoroethyl)-6-bromo-1H-indol-1-yl)-2-methylpropan-1-one

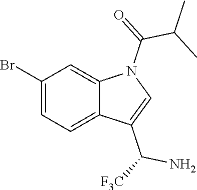 [SMILES: CC(C)C(=O)n1cc([C@H](N)C(F)(F)F)c2ccc(Br)cc21]

A mixture of (S)—N—((S)-1-(6-bromo-1-isobutyryl-1H-indol-3-yl)-2,2,2-trifluoroethyl)-2-methylpropane-2-sulfinamide of above step in 4M HCl(g)/methanol (25 mL) was stirred at room temperature for 2 h. The mixture was basified to PH>8 with sat. NaHCO3, extracted with EtOAC twice. The combined phases was washed with water, dried over Na2SO4, filtered, and concentrated under reduced pressure. The residue was purified by chromatography on silica gel (Petroleum ether/EtOAc) to give (S)-1-(3-(1-amino-2,2,2-trifluoroethyl)-6-bromo-1H-indol-1-yl)-2-methylpropan-1-one (504 mg, 1.39 mmol, 28.44% yield) as yellow oil.

### Step 3: (S)-1-(3-(1-amino-2,2,2-trifluoroethyl)-6-(2-(trifluoromethyl)phenyl)-1H-indol-1-yl)-2-methylpropan-1-one

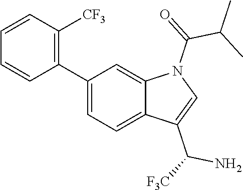 [SMILES: CC(C)C(=O)n1cc([C@H](N)C(F)(F)F)c2ccc(-c3ccccc3C(F)(F)F)cc21]

A mixture of(S)-1-(3-(1-amino-2,2,2-trifluoroethyl)-6-bromo-1H-indol-1-yl)-2-methylpropan-1-one (504 mg, 1.39 mmol), [2-(trifluoromethyl)phenyl]boronic acid (342.65 mg, 1.80 mmol), Pd(dppf)Cl2 (203.09 mg, 277.55 μmol) and Na2CO3 (441.26 mg, 4.16 mmol) in dioxane (12 mL) and water (3 mL) was stirred at 80° C. for 5 h under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-50%) to get (S)-1-(3-(1-amino-2,2,2-trifluoroethyl)-6-(2-(trifluoromethyl)phenyl)-1H-indol-1-yl)-2-methylpropan-1-one (460 mg, 187.92 μmol, 13.54% yield, 17.50% purity) as brown oil.

### Step 4: (S)—N-(2,2,2-trifluoro-1-(1-isobutyryl-6-(2-(trifluoromethyl)phenyl)-1H-indol-3-yl)ethyl)cyclobutanesulfonamide

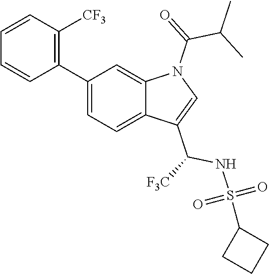 [SMILES: CC(C)C(=O)n1cc([C@H](NS(=O)(=O)C2CCC2)C(F)(F)F)c2ccc(-c3ccccc3C(F)(F)F)cc21]

A mixture of (S)-1-(3-(1-amino-2,2,2-trifluoroethyl)-6-(2-(trifluoromethyl)phenyl)-1H-indol-1-yl)-2-methylpropan-1-one (300 mg, 122.56 μmol) and cyclobutanesulfonyl chloride (217.92 mg, 1.41 mmol) in anhydrous pyridine (0.5 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC twice to get (S)—N-(2,2,2-trifluoro-1-(1-isobutyryl-6-(2-(trifluoromethyl)phenyl)-1H-indol-3-yl)ethyl)cyclobutanesulfonamide (12.46 mg, 22.80 μmol, 18.60% yield) as a white solid. ¹H NMR (400 MHz, CDCl₃) δ 8.52 (s, 1H), 7.73 (dd, J=19.1, 10.4 Hz, 3H), 7.56 (t, J=7.4 Hz, 1H), 7.48 (t, J=7.6 Hz, 1H), 7.36 (t, J=7.3 Hz, 2H), 5.46-5.34 (m, 1H), 5.00 (d, J=8.4 Hz, 1H), 3.85 (p, J=8.2 Hz, 1H), 3.31 (dt, J=13.5, 6.7 Hz, 1H), 2.61-2.38 (m, 2H), 2.26 (d, J=5.5 Hz, 2H), 2.05-1.86 (m, 2H), 1.44-1.21 (m, 6H). LCMS: 563.8 m/z [M+18]⁺.

### Example: 505: (S)—N-(2,2,2-trifluoro-1-(5-fluoro-1-isobutyryl-6-(2-(trifluoromethyl)phenyl)-1H-indol-3-yl)ethyl)cyclobutanesulfonamide (505)

### Step 1: (S)—N—((S)-1-(6-bromo-5-fluoro-1H-indol-3-yl)-2,2,2-trifluoroethyl)-2-methylpropane-2-sulfinamide

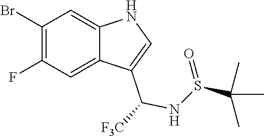 [SMILES: CC(C)(C)[S@](=O)N[C@@H](c1c[nH]c2cc(Br)c(F)cc12)C(F)(F)F]

To a stirred solution of 6-bromo-5-fluoro-1H-indole (1.2 g, 5.61 mmol) in anhydrous CH2Cl2 (10 mL) was added BF3·OEt2 (1.19 g, 8.41 mmol, 1.04 mL) slowly at −10° C. under Nitrogen, followed by addition of a solution of (S,E)-2-methyl-N-(2,2,2-trifluoroethylidene)propane-2-sulfinamide in DCM. The resulting mixture was raised to room temperature for 3 h. Then the mixture was added water. The mixture was separated organic phase, the aqueous phase was extracted with CH2Cl2, the combined organic phases were washed with brine, dried with anhydrous Na2SO4 and filtered. The filtrate was concentrated to give the crude product, which was purified by silica gel column (EA/PE=0-100%) to get (S)—N—((S)-1-(6-bromo-5-fluoro-1H-indol-3-yl)-2,2,2-trifluoroethyl)-2-methylpropane-2-sulfinamide (1.42 g, 3.42 mmol, 60.99% yield) as yellow oil.

### Step 2: (S)—N—((S)-1-(6-bromo-5-fluoro-1-isobutyryl-1H-indol-3-yl)-2,2,2-trifluoroethyl)-2-methylpropane-2-sulfinamide

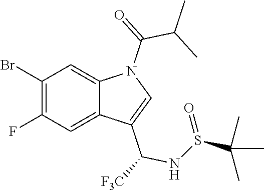 [SMILES: CC(C)C(=O)n1cc([C@H](N[S@@](=O)C(C)(C)C)C(F)(F)F)c2cc(F)c(Br)cc21]

A mixture of (S)—N—((S)-1-(6-bromo-5-fluoro-1H-indol-3-yl)-2,2,2-trifluoroethyl)-2-methylpropane-2-sulfinamide (1.29 g, 3.10 mmol), 2-methylpropanoyl chloride (396.60 mg, 3.72 mmol, 389.97 μL), N,N-dimethylpyridin-4-amine (75.79 mg, 620.36 μmol) and Triethylamine (627.74 mg, 6.20 mmol, 864.66 μL) in anhydrous CH2Cl2 (25 mL) was stirred at room temperature for overnight. The mixture was concentrated to get oil, which was purified by prep-HPLC to get (S)—N—((S)-1-(6-bromo-5-fluoro-1-isobutyryl-1H-indol-3-yl)-2,2,2-trifluoroethyl)-2-methylpropane-2-sulfinamide (580 mg, 1.20 mmol, 38.53% yield) as light-yellow gum.

### Step 3: (S)-1-(3-(1-amino-2,2,2-trifluoroethyl)-6-bromo-5-fluoro-1H-indol-1-yl)-2-methylpropan-1-one hydrochloride

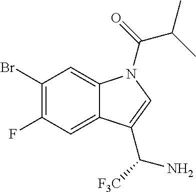 [SMILES: CC(C)C(=O)n1cc([C@H](N)C(F)(F)F)c2cc(F)c(Br)cc21]

A mixture of (S)—N—((S)-1-(6-bromo-5-fluoro-1-isobutyryl-1H-indol-3-yl)-2,2,2-trifluoroethyl)-2-methylpropane-2-sulfinamide (580 mg, 1.20 mmol) in 4M HCl(g)/methanol (12 mL) was stirred at room temperature for 1.5 h. The mixture was concentrated to get (S)-1-(3-(1-amino-2,2,2-trifluoroethyl)-6-bromo-5-fluoro-1H-indol-1-yl)-2-methylpropan-1-one hydrochloride (499.08 mg, 1.20 mmol, 100.00% yield) as a off-white solid.

### Step 4: (S)-1-(3-(1-amino-2,2,2-trifluoroethyl)-5-fluoro-6-(2-(trifluoromethyl)phenyl)-1H-indol-1-yl)-2-methylpropan-1-one

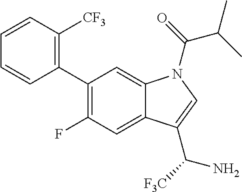 [SMILES: CC(C)C(=O)n1cc([C@H](N)C(F)(F)F)c2cc(F)c(-c3ccccc3C(F)(F)F)cc21]

A mixture of (S)-1-(3-(1-amino-2,2,2-trifluoroethyl)-6-bromo-5-fluoro-1H-indol-1-yl)-2-methylpropan-1-one hydrochloride (455 mg, 1.09 mmol), [2-(trifluoromethyl)phenyl]boronic acid (269.00 mg, 1.42 mmol), Pd(dppf)Cl2 (159.44 mg, 217.90 μmol) and Na2CO3 (577.37 mg, 5.45 mmol) in dioxane (10 mL) and water (2.5 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-50%) to get (S)-1-(3-(1-amino-2,2,2-trifluoroethyl)-5-fluoro-6-(2-(trifluoromethyl)phenyl)-1H-indol-1-yl)-2-methylpropan-1-one (278 mg, 622.81 μmol, 57.17% yield) as a yellow solid.

### Step 5: (S)—N-(2,2,2-trifluoro-1-(5-fluoro-1-isobutyryl-6-(2-(trifluoromethyl)phenyl)-1H-indol-3-yl)ethyl)cyclobutanesulfonamide

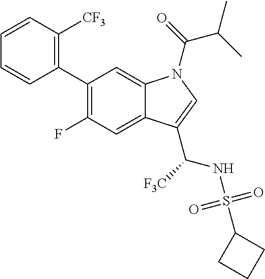 [SMILES: CC(C)C(=O)n1cc([C@H](NS(=O)(=O)C2CCC2)C(F)(F)F)c2cc(F)c(-c3ccccc3C(F)(F)F)cc21]

A mixture of (S)-1-(3-(1-amino-2,2,2-trifluoroethyl)-5-fluoro-6-(2-(trifluoromethyl)phenyl)-1H-indol-1-yl)-2-methylpropan-1-one (140 mg, 313.65 μmol) and cyclobutanesulfonyl chloride (145.48 mg, 940.94 μmol) in anhydrous pyridine (0.4 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(2,2,2-trifluoro-1-(5-fluoro-1-isobutyryl-6-(2-(trifluoromethyl)phenyl)-1H-indol-3-yl)ethyl)cyclobutanesulfonamide (72 mg, 127.54 μmol, 40.66% yield) as a white solid. ¹H NMR (400 MHz, DMSO) δ 8.67 (s, 1H), 8.47 (d, J=5.9 Hz, 1H), 8.31 (d, J=6.6 Hz, 1H), 7.95 (dd, J=9.8, 5.1 Hz, 1H), 7.89 (d, J=7.5 Hz, 1H), 7.77 (t, J=7.4 Hz, 1H), 7.70 (t, J=7.6 Hz, 1H), 7.50 (d, J=7.5 Hz, 1H), 5.67 (s, 1H), 3.81 (dt, J=16.5, 8.3 Hz, 1H), 3.43 (dd, J=13.6, 6.8 Hz, 1H), 2.34-2.21 (m, 2H), 2.11-1.96 (m, 2H), 1.82 (dd, J=16.4, 8.1 Hz, 2H), 1.25 (d, J=6.4 Hz, 6H). LCMS: 582.2 m/z [M+18]⁺.

### Example 506: (S)—N-(1-(6-cyclopentyl-1-neopentyl-1H-indol-3-yl)-2,2,2-trifluoroethyl)cyclobutanesulfonamide (506)

### Step 1: (S)-1-(6-cyclopentenyl-1-neopentyl-1H-indol-3-yl)-2,2,2-trifluoroethanamine

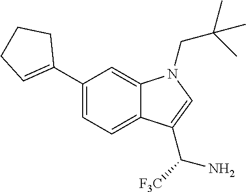 [SMILES: CC(C)(C)Cn1cc([C@H](N)C(F)(F)F)c2ccc(C3=CCCC3)cc21]

A mixture of (S)-1-(6-bromo-1-neopentyl-1H-indol-3-yl)-2,2,2-trifluoroethanamine (400 mg, 1.10 mmol), 2-(cyclopenten-1-yl)-4,4,5,5-tetramethyl-1,3,2-dioxaborolane (320.60 mg, 1.65 mmol), Pd(dppf)Cl2 (161.16 mg, 220.26 μmol) and Cs2CO3 (897.04 mg, 2.75 mmol) in dioxane (16 mL) and water (4 mL) was stirred at 80° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-50%) to get (S)-1-(6-cyclopentenyl-1-neopentyl-1H-indol-3-yl)-2,2,2-trifluoroethanamine (350 mg, 998.80 μmol, 90.69% yield) as light-yellow oil.

### Step 2: (S)-1-(6-cyclopentyl-1-neopentyl-1H-indol-3-yl)-2,2,2-trifluoroethanamine

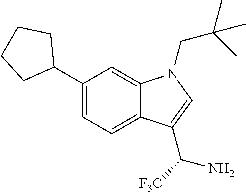 [SMILES: CC(C)(C)Cn1cc([C@H](N)C(F)(F)F)c2ccc(C3CCCC3)cc21]

A mixture of (S)-1-(6-cyclopentenyl-1-neopentyl-1H-indol-3-yl)-2,2,2-trifluoroethanamine (205 mg, 585.01 μmol) 10% Pd/C (41 mg, 585.01 μmol) in Methanol (8 mL) was stirred at room temperature overnight under hydrogen. The mixture was filtered, The filtrate was concentrated to get (S)-1-(6-cyclopentyl-1-neopentyl-1H-indol-3-yl)-2,2,2-trifluoroethanamine (170 mg, 482.36 μmol, 82.45% yield) as light-yellow oil.

### Step 3: (S)—N-(1-(6-cyclopentyl-1-neopentyl-1H-indol-3-yl)-2,2,2-trifluoroethyl)cyclobutanesulfonamide

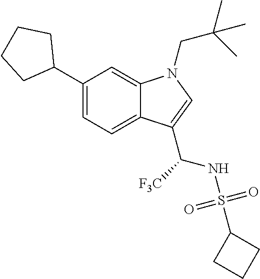 [SMILES: CC(C)(C)Cn1cc([C@H](NS(=O)(=O)C2CCC2)C(F)(F)F)c2ccc(C3CCCC3)cc21]

A mixture of (S)-1-(6-cyclopentyl-1-neopentyl-1H-indol-3-yl)-2,2,2-trifluoroethanamine (160 mg, 453.98 μmol) and cyclobutanesulfonyl chloride (180.50 mg, 817.17 μmol) in anhydrous pyridine (0.4 mL) was stirred at 100° C. for 4 hours in the microwave. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(1-(6-cyclopentyl-1-neopentyl-1H-indol-3-yl)-2,2,2-trifluoroethyl)cyclobutanesulfonamide (19.30 mg, 41.01 μmol, 9.03% yield) as a white solid. ¹H NMR (400 MHz, MeOD) δ 7.56 (d, J=8.3 Hz, 1H), 7.30 (d, J=12.3 Hz, 2H), 7.04 (dd, J=8.3, 1.3 Hz, 1H), 5.27 (t, J=8.0 Hz, 1H), 4.7-4.8 (m, 1H), 3.95 (dd, J=11.1, 6.5 Hz, 2H), 3.60 (t, J=8.2 Hz, 1H), 3.13 (t, J=8.4 Hz, 1H), 2.29 (ddd, J=20.9, 8.8, 3.4 Hz, 2H), 2.16-2.05 (m, 2H), 2.00 (d, J=2.5 Hz, 1H), 1.93-1.81 (m, 3H), 1.80-1.63 (m, 6H), 0.98 (d, J=8.2 Hz, 9H): 470.9 m/z [M+1]⁺.

### Example 507: N-(1-(1-neopentyl-6-(2-(trifluoromethyl)phenyl)-1H-indol-3-yl)ethyl)cyclobutanesulfonamide (507)

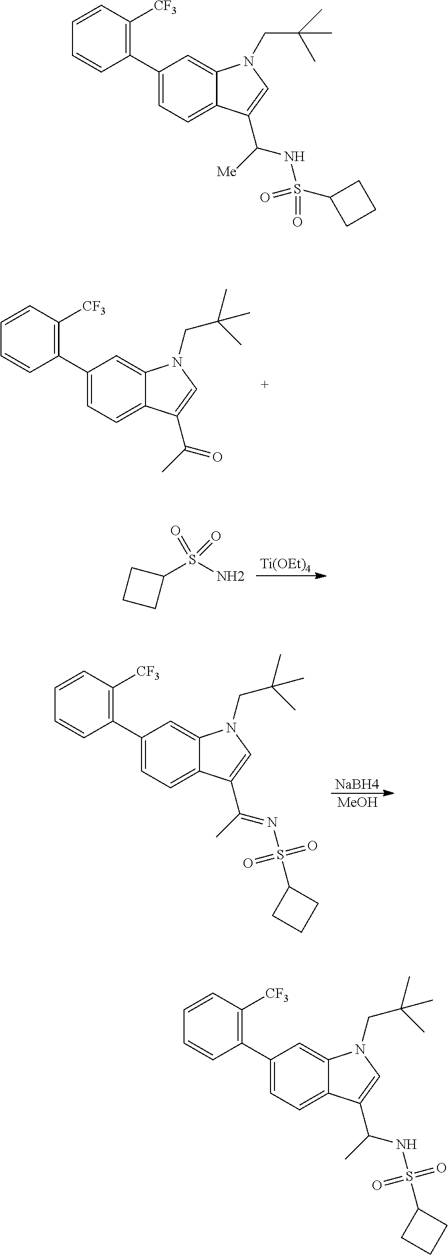

### Step 1: (E)-N-(1-(1-neopentyl-6-(2-(trifluoromethyl)phenyl)-1H-indol-3-yl)ethylidene)cyclobutanesulfonamide

The title compound was prepared following the same general protocol as described for Step 4, Example 113, using 1-(1-neopentyl-6-(2-(trifluoromethyl)phenyl)-1H-indol-3-yl)ethan-1-one and cyclobutanesulfonamide. ESI-MS (m/z): 491.2 [M+1]⁺.

### Step 2: N-(1-(1-neopentyl-6-(2-(trifluoromethyl)phenyl)-1H-indol-3-yl)ethyl)cyclobutanesulfonamide

The title compound was prepared following the same general protocol as described for Step 3, Example 420 (Compound 420), using (E)-N-(1-(1-neopentyl-6-(2-(trifluoromethyl)phenyl)-1H-indol-3-yl)ethylidene)cyclobutanesulfonamide. ESI-MS (m/z): 515.2 [M+Na]⁺.

### Example 508: N-(1-(5-fluoro-1-neopentyl-6-(2-(trifluoromethyl)phenyl)-1H-indol-3-yl)ethyl)cyclobutanesulfonamide (508)

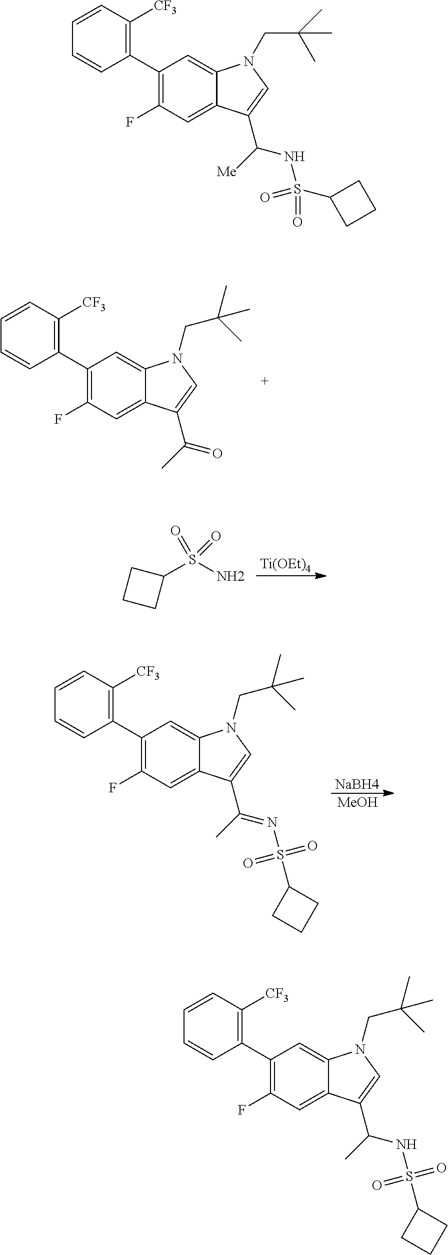

### Step 1: (E)-N-(1-(5-fluoro-1-neopentyl-6-(2-(trifluoromethyl)phenyl)-1H-indol-3-yl)ethylidene)cyclobutanesulfonamide

The title compound was prepared following the same general protocol as described for Step 4, Example 113, using 1-(5-fluoro 1-neopentyl-6-(2-(trifluoromethyl)phenyl)-1H-indol-3-yl)ethan-1-one and cyclobutanesulfonamide. ESI-MS (m/z): 509.2 [M+1]⁺.

### Step 2: N-(1-(5-fluoro 1-neopentyl-6-(2-(trifluoromethyl)phenyl)-1H-indol-3-yl)ethyl)cyclobutanesulfonamide

The title compound was prepared following the same general protocol as described for Step 3, Example 420 (Compound 420), using (E)-N-(1-(5-fluoro 1-neopentyl-6-(2-(trifluoromethyl)phenyl)-1H-indol-3-yl)ethylidene)cyclobutanesulfonamide. ESI-MS (m/z): 533.2 [M+Na]⁺.

### Example 509: N-(1-(1-neopentyl-6-(2-(trifluoromethyl)phenyl)-1H-indol-3-yl)ethyl-2,2,2-d3)cyclobutanesulfonamide (509)

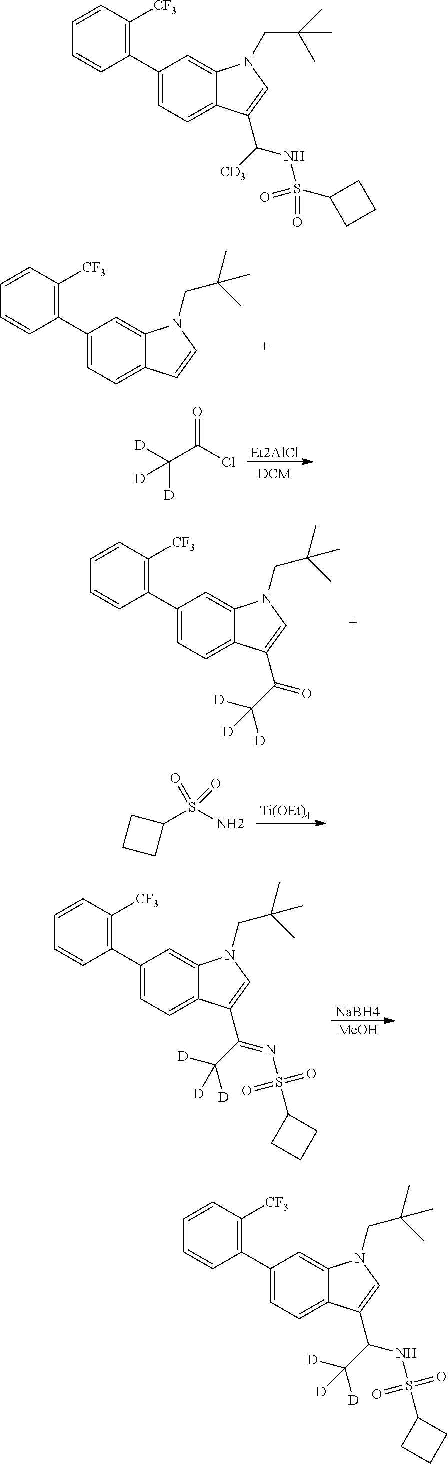

### Step 1: 1-(1-neopentyl-6-(2-(trifluoromethyl)phenyl)-1H-indol-3-yl)ethan-1-one-2,2,2-d3

The title compound was prepared following the same general protocol as described for Step 1, Example 87, using 1-neopentyl-6-(2-(trifluoromethyl)phenyl)-1H-indole and acetyl-d3 chloride. ESI-MS (m/z): 377.2 [M+1]⁺.

### Step 2: (E)-N-(1-(1-neopentyl-6-(2-(trifluoromethyl)phenyl)-1H-indol-3-yl)ethylidene-2,2,2-d3)cyclobutanesulfonamide

The title compound was prepared following the same general protocol as described for Step 4, Example 113, using 1-(1-neopentyl-6-(2-(trifluoromethyl)phenyl)-1H-indol-3-yl)ethan-1-one-2,2,2-d3. ESI-MS (m/z): 494.2 [M+1]⁺.

### Step 3: N-(1-(1-neopentyl-6-(2-(trifluoromethyl)phenyl)-1H-indol-3-yl)ethyl-2,2,2-d3)cyclobutanesulfonamide

The title compound was prepared following the same general protocol as described for Step 3, Example 420 (Compound 420), using (E)-N-(1-(1-neopentyl-6-(2-(trifluoromethyl)phenyl)-1H-indol-3-yl)ethylidene-2,2,2-d3)cyclobutanesulfonamide. ESI-MS (m/z): 496.2 [M+1]⁺.

### Example 510: N-(1-(1-(cyclopentylmethyl)-6-(2-(trifluoromethyl)phenyl)-1H-indol-3-yl)ethyl)cyclobutanesulfonamide (510)

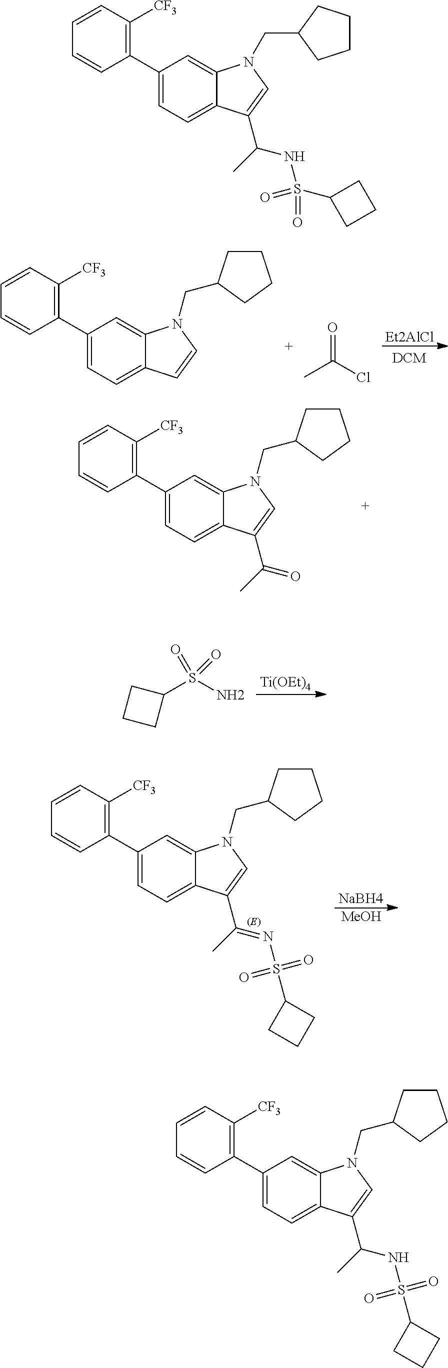

### Step 1: 1-(1-(cyclopentylmethyl)-6-(2-(trifluoromethyl)phenyl)-1H-indol-3-yl)ethan-1-one

The title compound was prepared following the same general protocol as described for Step 1, Example 87, using 1-(cyclopentylmethyl)-6-(2-(trifluoromethyl)phenyl)-1H-indole and acetyl chloride. ESI-MS (m/z): 386.2 [M+1]⁺.

### Step 2: (E)-N-(1-(1-(cyclopentylmethyl)-6-(2-(trifluoromethyl)phenyl)-1H-indol-3-yl)ethylidene)cyclobutanesulfonamide

The title compound was prepared following the same general protocol as described for Step 4, Example 113, using 1-(1-(cyclopentylmethyl)-6-(2-(trifluoromethyl)phenyl)-1H-indol-3-yl)ethan-1-one. ESI-MS (m/z): 503.2 [M+1]⁺.

### Step 3: N-(1-(1-(cyclopentylmethyl)-6-(2-(trifluoromethyl)phenyl)-1H-indol-3-yl)ethyl)cyclobutanesulfonamide

The title compound was prepared following the same general protocol as described for Step 3, Example 420 (Compound 420), using (E)-N-(1-(1-(cyclopentylmethyl)-6-(2-(trifluoromethyl)phenyl)-1H-indol-3-yl)ethylidene)cyclobutanesulfonamide. ESI-MS (m/z): 527.2 [M+Na]⁺.

### Example 511: N-((1S)-2,2,2-trifluoro-1-(5-fluoro-6-(2-fluoro-6-(trifluoromethyl)phenyl)-1-neopentyl-1H-indol-3-yl)ethyl)cyclobutanesulfonamide (511)

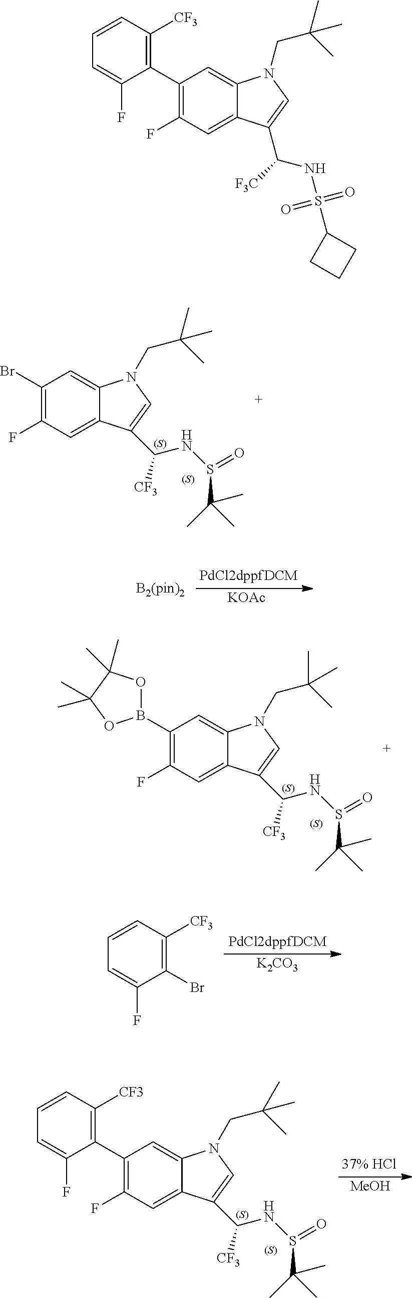

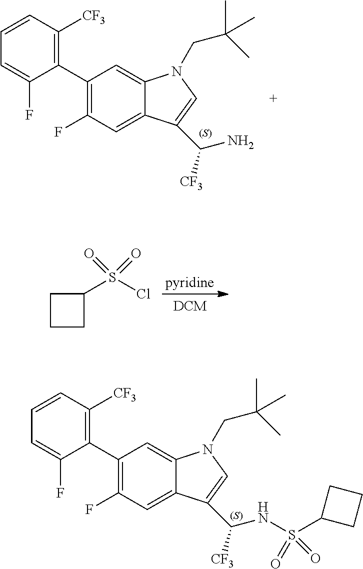

### Step 1: (S)-2-methyl-N—((S)-2,2,2-trifluoro-1-(5-fluoro-1-neopentyl-6-(4,4,5,5-tetramethyl-1,3,2-dioxaborolan-2-yl)-1H-indol-3-yl)ethyl)propane-2-sulfinamide

The title compound was prepared following the same general protocol as described for Step 2, Example 33, using (S)—N—((S)-1-(6-bromo-5-fluoro-1-neopentyl-1H-indol-3-yl)-2,2,2-trifluoroethyl)-2-methylpropane-2-sulfinamide. ESI-MS (m/z): 548.2 [M+1]⁺.

### Step 2: (S)-2-methyl-N-((1S)-2,2,2-trifluoro-1-(5-fluoro-6-(2-fluoro-6-(trifluoromethyl)phenyl)-1-neopentyl-1H-indol-3-yl)ethyl)propane-2-sulfinamide

The title compound was prepared following the same general protocol as described for Step 5, Example 1, using (S)-2-methyl-N—((S)-2,2,2-trifluoro-1-(5-fluoro-1-neopentyl-6-(4,4,5,5-tetramethyl-1,3,2-dioxaborolan-2-yl)-1H-indol-3-yl)ethyl)propane-2-sulfinamide and 2-bromo-1-fluoro-3-(trifluoromethyl)benzene. ESI-MS (m/z): 569.2 [M+1]⁺.

### Step 3: (1S)-2,2,2-trifluoro-1-(5-fluoro-6-(2-fluoro-6-(trifluoromethyl)phenyl)-1-neopentyl-1H-indol-3-yl)ethan-1-amine

The title compound was prepared following the same general protocol as described for Step 4, Example 116, using (S)-2-methyl-N-((1S)-2,2,2-trifluoro-1-(5-fluoro-6-(2-fluoro-6-(trifluoromethyl)phenyl)-1-neopentyl-1H-indol-3-yl)ethyl)propane-2-sulfinamide. ESI-MS (m/z): 448.2 [M-NH₂]⁺.

### Step 4: N-((1S)-2,2,2-trifluoro-1-(5-fluoro-6-(2-fluoro-6-(trifluoromethyl)phenyl)-1-neopentyl-1H-indol-3-yl)ethyl)cyclobutanesulfonamide

The title compound was prepared following the same general protocol as described for Step 5, Example 116, using (1S)-2,2,2-trifluoro-1-(5-fluoro-6-(2-fluoro-6-(trifluoromethyl)phenyl)-1-neopentyl-1H-indol-3-yl)ethan-1-amine and cyclobutanesulfonyl chloride. ESI-MS (m/z): 583.2 [M+1]⁺.

### Example 512: (S)—N-(2,2,2-trifluoro-1-(1-((1-methylcyclopropyl)methyl)-6-(2-(trifluoromethyl)phenyl)-1H-indol-3-yl)ethyl)cyclobutanesulfonamide (512)

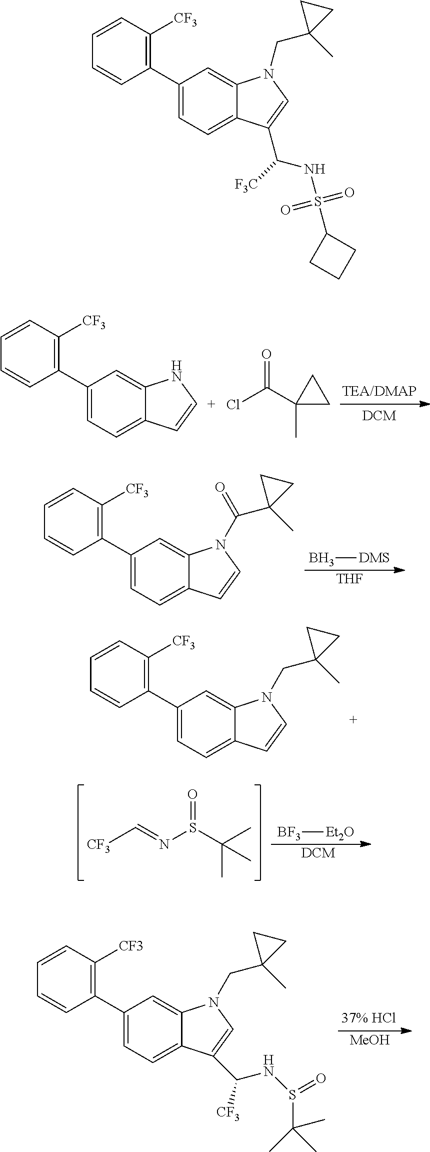

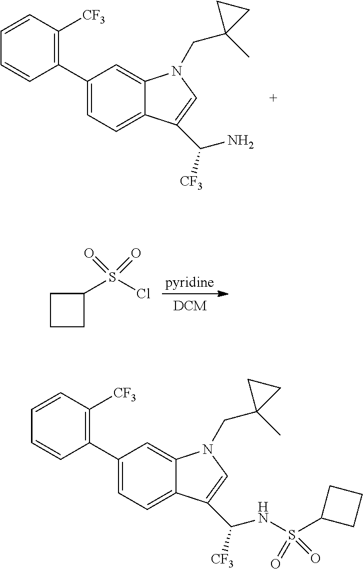

### Step 1: (1-methylcyclopropyl)(6-(2-(trifluoromethyl)phenyl)-1H-indol-1-yl)methanone

The title compound was prepared following the same general protocol as described for Example 34, using 6-(2-(trifluoromethyl)phenyl)-1H-indole and 1-methylcyclopropane-1-carbonyl chloride. ESI-MS (m/z): 344.1 [M+1]⁺.

### Step 2: 1-((1-methylcyclopropyl)methyl)-6-(2-(trifluoromethyl)phenyl)-1H-indole

The title compound was prepared following the same general protocol as described for Step 3, Example 1, using (1-methylcyclopropyl)(6-(2-(trifluoromethyl)phenyl)-1H-indol-1-yl)methanone. ESI-MS (m/z): 330.1 [M+1]⁺.

### Step 3: (S)-2-methyl-N—((S)-2,2,2-trifluoro-1-(1-((1-methylcyclopropyl)methyl)-6-(2-(trifluoromethyl)phenyl)-1H-indol-3-yl)ethyl)propane-2-sulfinamide

The title compound was prepared following the same general protocol as described for Step 3, Example 116, using 1-((1-methylcyclopropyl)methyl)-6-(2-(trifluoromethyl)phenyl)-1H-indole. ESI-MS (m/z): 531.2 [M+1]⁺.

### Step 4: (S)-2,2,2-trifluoro-1-(1-((1-methylcyclopropyl)methyl)-6-(2-(trifluoromethyl)phenyl)-1H-indol-3-yl)ethan-1-amine

The title compound was prepared following the same general protocol as described for Step 4, Example 116, using (S)-2-methyl-N—((S)-2,2,2-trifluoro-1-(1-((1-methylcyclopropyl)methyl)-6-(2-(trifluoromethyl)phenyl)-1H-indol-3-yl)ethyl)propane-2-sulfinamide. ESI-MS (m/z): 410.1 [M-NH₂]⁺.

### Step 5: (S)—N-(2,2,2-trifluoro-1-(1-((1-methylcyclopropyl)methyl)-6-(2-(trifluoromethyl)phenyl)-1H-indol-3-yl)ethyl)cyclobutanesulfonamide

The title compound was prepared following the same general protocol as described for Step 5, Example 116, using (S)-2,2,2-trifluoro-1-(1-((1-methylcyclopropyl)methyl)-6-(2-(trifluoromethyl)phenyl)-1H-indol-3-yl)ethan-1-amine and cyclobutanesulfonyl chloride. ESI-MS (m/z): 545.2 [M+1]⁺.

### Example 513: (S)—N-(2,2-difluoro-1-(1-neopentyl-6-(2-(trifluoromethyl)phenyl)-1H-indol-3-yl)ethyl)cyclobutanesulfonamide (513)

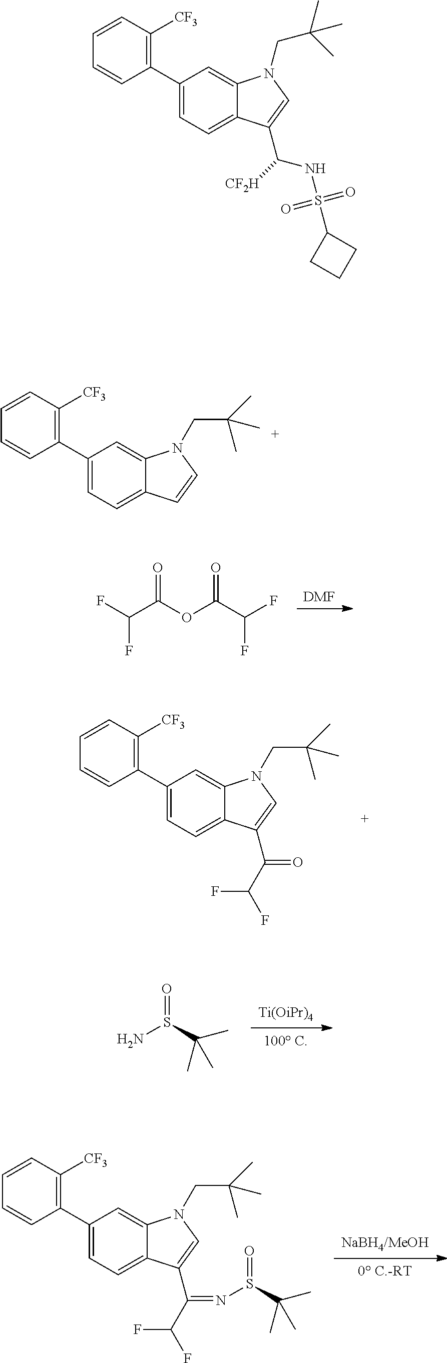

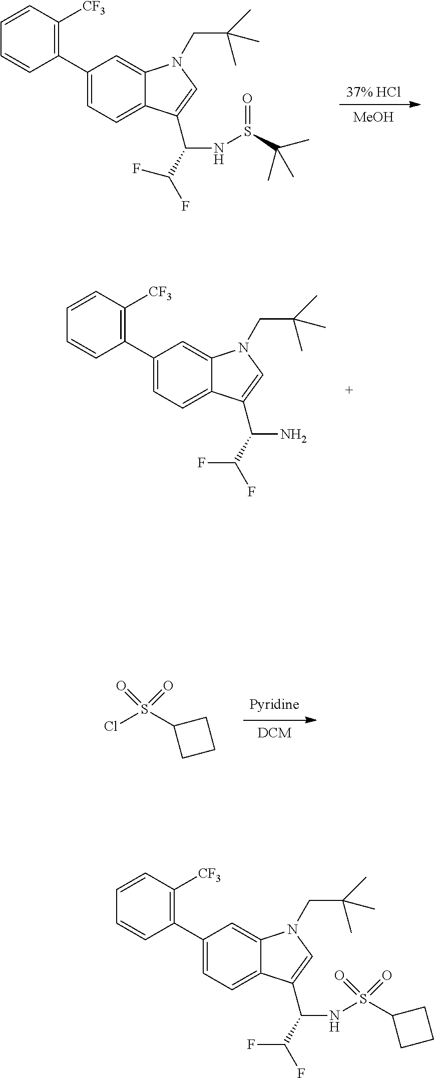

### Step 1: 2,2-difluoro-1-(1-neopentyl-6-(2-(trifluoromethyl)phenyl)-1H-indol-3-yl)ethan-1-one

The title compound was prepared following the same general protocol as described for Step 1, Example 128, using 1-neopentyl-6-(2-(trifluoromethyl)phenyl)-1H-indole and 2,2-difluoroacetic anhydride. ESI-MS (m/z): 410.1 [M+1]⁺. ¹HNMR (CDCl3, 400 MHz) δ 8.42 (dd, J1=8.1 Hz, J2=0.6 Hz, 1H), 8.04 (t, J=1.7 Hz, 1H), 7.77 (d, J=7.4 Hz, 1H), 7.58 (t, J=7.4 Hz, 1H), 7.49 (t, J=7.4 Hz, 1H), 7.38 (s, 1H), 7.37 (d, J=7.5 Hz, 1H), 7.29 (dd, J1=7.9 Hz, J2=1.6 Hz, 1H), 6.14 (t, J=54 Hz, 1H), 3.98 (s, 2H), 1.04 (s, 9H).

### Step 2: (S,E)-N-(2,2-difluoro-1-(1-neopentyl-6-(2-(trifluoromethyl)phenyl)-1H-indol-3-yl)ethylidene)-2-methylpropane-2-sulfinamide

The title compound was prepared following the same general protocol as described for Step 4, Example 113, using 2,2-difluoro-1-(1-neopentyl-6-(2-(trifluoromethyl)phenyl)-1H-indol-3-yl)ethan-1-one. ESI-MS (m/z): 513.2 [M+1]⁺.

### Step 3: (S)—N—((S)-2,2-difluoro-1-(1-neopentyl-6-(2-(trifluoromethyl)phenyl)-1H-indol-3-yl)ethyl)-2-methylpropane-2-sulfinamide

The title compound was prepared following the same general protocol as described for Step 3, Example 420 (Compound 420), using (S,E)-N-(2,2-difluoro-1-(1-neopentyl-6-(2-(trifluoromethyl)phenyl)-1H-indol-3-yl)ethylidene)-2-methylpropane-2-sulfinamide. ESI-MS (m/z): 515.2 [M+1]⁺.

### Step 4: (S)-2,2-difluoro-1-(1-neopentyl-6-(2-(trifluoromethyl)phenyl)-1H-indol-3-yl)ethan-1-amine

The title compound was prepared following the same general protocol as described for Step 4, Example 116, using (S)—N—((S)-2,2-difluoro-1-(1-neopentyl-6-(2-(trifluoromethyl)-phenyl)-1H-indol-3-yl)ethyl)-2-methylpropane-2-sulfinamide. ESI-MS (m/z): 394.2 [M-NH₂]⁺.

### Step 5: (S)—N-(2,2-difluoro-1-(1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2,2-difluoro-1-(1-neopentyl-6-(2-(trifluoromethyl)phenyl)-1H-indol-3-yl)ethan-1-amine and cyclobutanesulfonyl chloride. ESI-MS (m/z): 529.2 [M+1]⁺.

### Example 514: (1S,2S)-2-methyl-N—((S)-2,2,2-trifluoro-1-(1-neopentyl-6-(2-(trifluoromethyl)phenyl)-1H-indol-3-yl)ethyl)cyclopropane-1-sulfonamide (514)

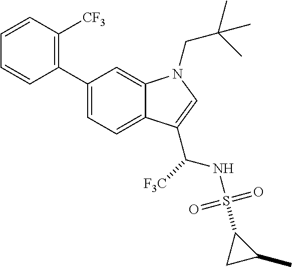

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and (1R,2S)-2-methylcyclopropane-1-sulfonyl chloride. ¹HNMR (CDCl₃, 400 MHz) δ 7.77-7.72 (m, 2H), 7.56 (t, J=8.0 Hz, 1H), 7.47 (t, J=8.0 Hz, 1H), 7.36 (d, J=8.0 Hz, 1H), 7.32 (s, 1H), 7.24 (s, 1H), 7.13 (d, J=8.0 Hz, 1H), 5.42 (q, J=8.0 Hz, 1H), 5.00-4.99 (m, 1H), 3.88 (s, 2H), 2.10-2.06 (m, 1H), 1.58-1.46 (m, 1H), 1.14-1.25 (m, 1H), 0.98 (s, 9H), 0.90 (d, J=8.0 Hz, 3H), 0.71-0.65 (m, 1H)

### Example 515: 2,2-difluoro-N—((S)-2,2,2-trifluoro-1-(1-neopentyl-6-(2-(trifluoromethyl)phenyl)-1H-indol-3-yl)ethyl)cyclopropane-1-sulfonamide (515)

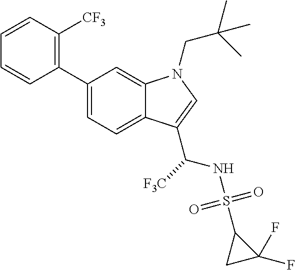 [SMILES: CC(C)(C)Cn1cc([C@H](NS(=O)(=O)C2CC2(F)F)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 2,2-difluorocyclopropane-1-sulfonyl chloride. HNMR (CDCl₃, 400 MHz) δ 7.76 (d, J=8.0 Hz, 1H), 7.69 (d, J=8.0 Hz, 1H), 7.56 (t, J=8.0 Hz, 1H), 7.48 (t, J=8.0 Hz, 1H), 7.37-7.33 (m, 2H), 7.25 (s, 1H), 7.16-7.13 (m, 1H), 5.46 (q, J=8.0 Hz, 1H), 5.40-5.36 (m, 1H), 3.90-3.70 (m, 4H), 2.10-2.06 (m, 1H), 0.98 (s, 9H)

### Example 516: (S)—N-(2-fluoro-1-(1-neopentyl-6-(2-(trifluoromethyl)phenyl)-1H-indol-3-yl)ethyl)cyclobutanesulfonamide (516)

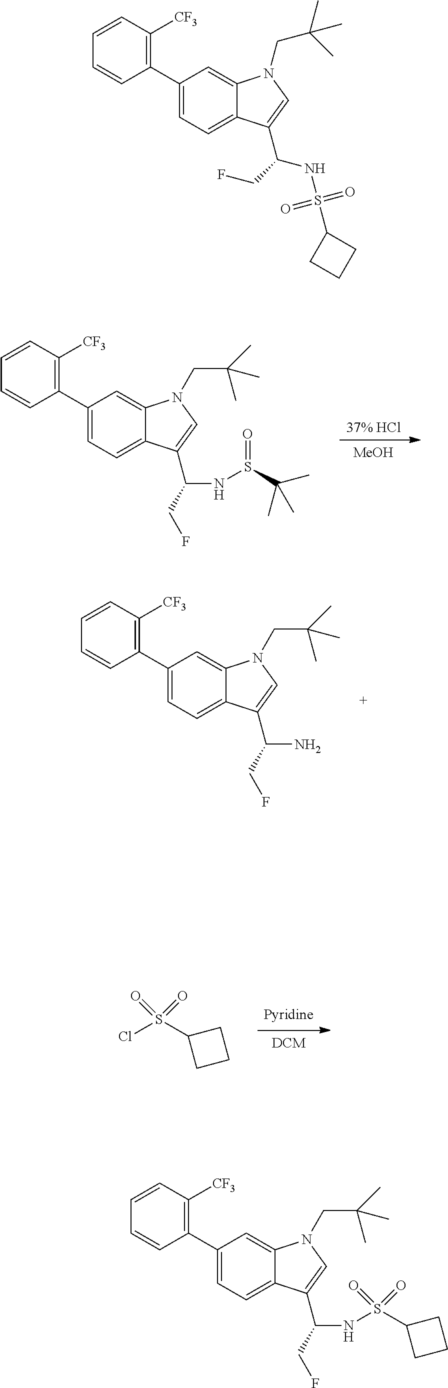

### Step 1: (S)-2-fluoro-1-(1-neopentyl-6-(2-(trifluoromethyl)phenyl)-1H-indol-3-yl)ethan-1-amine

The title compound was prepared following the same general protocol as described for Step 4, Example 116, using (S)—N—((S)-2-fluoro-1-(1-neopentyl-6-(2-(trifluoromethyl)phenyl)-1H-indol-3-yl)ethyl)-2-methylpropane-2-sulfinamide. ESI-MS (m/z): 376.2 [M-NH₂]⁺.

### Step 2: (S)—N-(2-fluoro-1-(1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2-fluoro-1-(1-neopentyl-6-(2-(trifluoromethyl)phenyl)-1H-indol-3-yl)ethan-1-amine and cyclobutanesulfonyl chloride. ESI-MS (m/z): 511.2 [M+1]⁺.

### Example 517: (S)—N-(1-(1-(2,2-dimethylpropyl-1,1-d2)-6-(2-(trifluoromethyl)phenyl)-1H-indol-3-yl)-2,2,2-trifluoroethyl)cyclobutanesulfonamide (517)

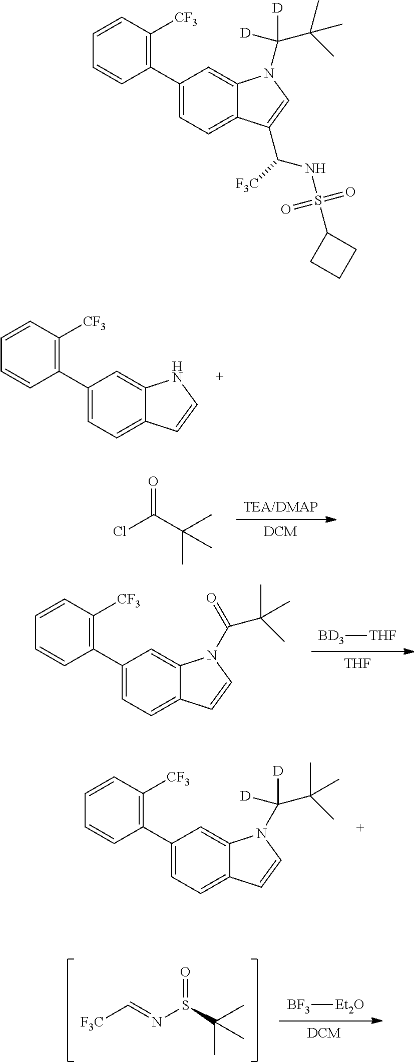

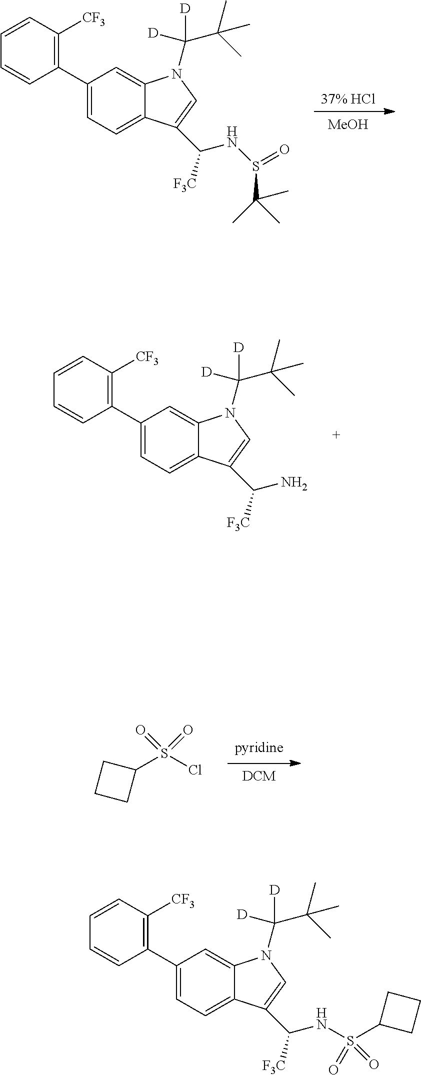

### Step 1: 2,2-dimethyl-1-(6-(2-(trifluoromethyl)phenyl)-1H-indol-1-yl)propan-1-one

The title compound was prepared following the same general protocol as described for Example 34, using 6-(2-(trifluoromethyl)phenyl)-1H-indole and pivaloyl chloride. ESI-MS (m/z): 346.1 [M+1]⁺.

### Step 2: 1-(2,2-dimethylpropyl-1,1-d2)-6-(2-(trifluoromethyl)phenyl)-1H-indole

The title compound was prepared following the same general protocol as described for Step 3, Example 1, using 2,2-dimethyl-1-(6-(2-(trifluoromethyl)phenyl)-1H-indol-1-yl)propan-1-one and borane-d3 THF solution. ESI-MS (m/z): 334.2 [M+1]⁺.

### Step 3: (S)—N—((S)-1-(1-(2,2-dimethylpropyl-1,1-d2)-6-(2-(trifluoromethyl)phenyl)-1H-indol-3-yl)-2,2,2-trifluoroethyl)-2-methylpropane-2-sulfinamide

The title compound was prepared following the same general protocol as described for Step 3, Example 116, using 1-(2,2-dimethylpropyl-1,1-d2)-6-(2-(trifluoromethyl)phenyl)-1H-indole. ESI-MS (m/z): 535.2 [M+1]⁺.

### Step 4: (S)-1-(1-(2,2-dimethylpropyl-1,1-d2)-6-(2-(trifluoromethyl)phenyl)-1H-indol-3-yl)-2,2,2-trifluoroethan-1-amine

The title compound was prepared following the same general protocol as described for Step 4, Example 116, using (S)—N—((S)-1-(1-(2,2-dimethylpropyl-1,1-d2)-6-(2-(trifluoromethyl)phenyl)-1H-indol-3-yl)-2,2,2-trifluoroethyl)-2-methylpropane-2-sulfinamide. ESI-MS (m/z): 414.2 [M-NH₂]⁺.

### Step 5: (S)—N-(1-(1-(2,2-dimethylpropyl-1,1-d2)-6-(2-(trifluoromethyl)phenyl)-1H-indol-3-yl)-2,2,2-trifluoroethyl)cyclobutanesulfonamide

The title compound was prepared following the same general protocol as described for Step 5, Example 116, using (S)-1-(1-(2,2-dimethylpropyl-1,1-d2)-6-(2-(trifluoromethyl)phenyl)-1H-indol-3-yl)-2,2,2-trifluoroethan-1-amine and cyclobutanesulfonyl chloride. ESI-MS (m/z): 549.2 [M+1]⁺.

### Example 518: (S)—N-(2,2-difluoro-1-(1-neopentyl-6-(2-(trifluoromethyl)phenyl)-1H-pyrrolo[2,3-b]pyridin-3-yl)ethyl)cyclobutanesulfonamide (518)

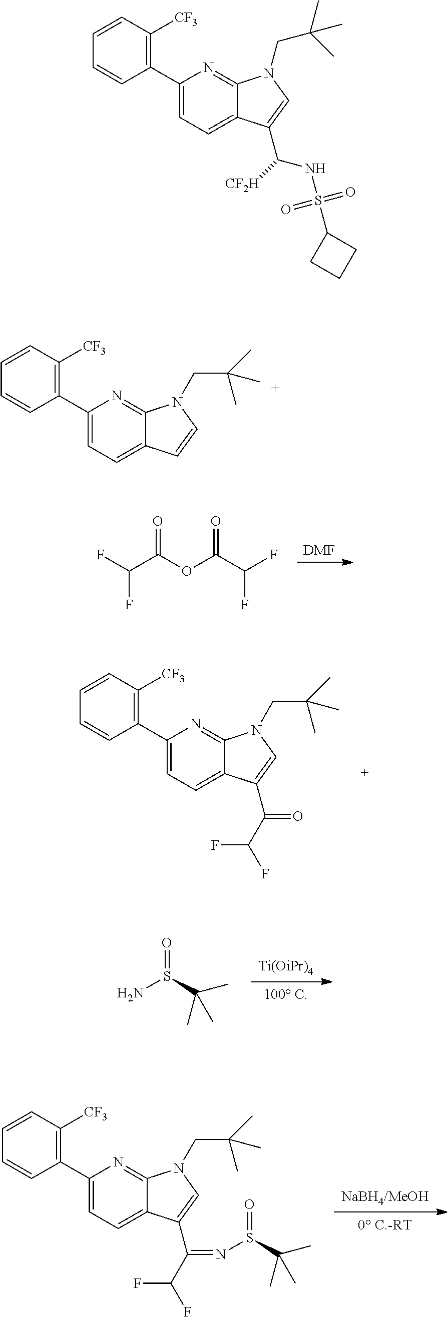

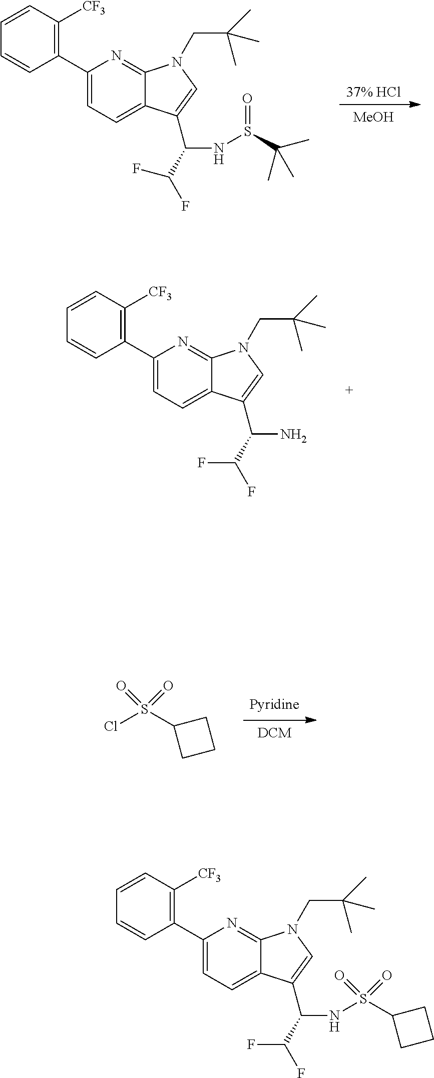

### Step 1: 2,2-difluoro-1-(1-neopentyl-6-(2-(trifluoromethyl)phenyl)-1H-pyrrolo[2,3-b]pyridin-3-yl)ethan-1-one

The title compound was prepared following the same general protocol as described for Step 1, Example 128, using 1-neopentyl-6-(2-(trifluoromethyl)phenyl)-1H-pyrrolo[2,3-b]pyridine and 2,2-difluoroacetic anhydride. ESI-MS (m/z): 411.1 [M+1]⁺. ¹HNMR (CDCl₃, 400 MHz) δ 8.67 (d, J=8.4 Hz, 1H), 8.19 (t, J=1.7 Hz, 1H), 7.40 (d, J=7.4 Hz, 1H), 7.63 (t, J=7.4 Hz, 1H), 7.50-7.56 (m, 2H), 7.41 (d, J=8.1 Hz, 1H), 6.14 (t, J=54 Hz, 1H), 4.21 (s, 2H), 1.01 (s, 9H).

### Step 2: (S,E)-N-(2,2-difluoro-1-(1-neopentyl-6-(2-(trifluoromethyl)phenyl)-1H-pyrrolo[2,3-b]pyridin-3-yl)ethylidene)-2-methylpropane-2-sulfinamide

The title compound was prepared following the same general protocol as described for Step 4, Example 113, using 2,2-difluoro-1-(1-neopentyl-6-(2-(trifluoromethyl)phenyl)-1H-pyrrolo[2,3-b]pyridin-3-yl)ethan-1-one. ESI-MS (m/z): 514.2 [M+1]⁺.

### Step 3: (S)—N—((S)-2,2-difluoro-1-(1-neopentyl-6-(2-(trifluoromethyl)phenyl)-1H-pyrrolo[2,3-b]pyridin-3-yl)ethyl)-2-methylpropane-2-sulfinamide

The title compound was prepared following the same general protocol as described for Step 3, Example 420 (Compound 420), using (S,E)-N-(2,2-difluoro-1-(1-neopentyl-6-(2-(trifluoromethyl)phenyl)-1H-pyrrolo[2,3-b]pyridin-3-yl)ethylidene)-2-methylpropane-2-sulfinamide. ESI-MS (m/z): 516.2 [M+1]⁺.

### Step 4: (S)-2,2-difluoro-1-(1-neopentyl-6-(2-(trifluoromethyl)phenyl)-1H-pyrrolo[2,3-b]pyridin-3-yl)ethan-1-amine

The title compound was prepared following the same general protocol as described for Step 4, Example 116, using (S)—N—((S)-2,2-difluoro-1-(1-neopentyl-6-(2-(trifluoromethyl)phenyl)-1H-pyrrolo[2,3-b]pyridin-3-yl)ethyl)-2-methylpropane-2-sulfinamide. ESI-MS (m/z): 395.2 [M-NH₂]⁺.

### Step 5: (S)—N-(2,2-difluoro-1-(1-neopentyl-6-(2-(trifluoromethyl)phenyl)-1H-pyrrolo[2,3-b]pyridin-3-yl)ethyl)cyclobutanesulfonamide

The title compound was prepared following the same general protocol as described for Step 5, Example 116, using (S)-2,2-difluoro-1-(1-neopentyl-6-(2-(trifluoromethyl)phenyl)-1H-pyrrolo[2,3-b]pyridin-3-yl)ethan-1-amine and cyclobutanesulfonyl chloride. ESI-MS (m/z): 530.2 [M+1]⁺.

### Example 519: (S)—N-(2,2,2-trifluoro-1-(1-neopentyl-6-(tetrahydro-2H-pyran-4-yl)-1H-indol-3-yl)ethyl)cyclobutanesulfonamide (519)

### Step 1: (S)-1-(6-(3,6-dihydro-2H-pyran-4-yl)-1-neopentyl-1H-indol-3-yl)-2,2,2-trifluoroethanamine

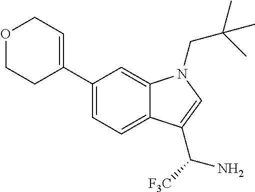 [SMILES: CC(C)(C)Cn1cc([C@H](N)C(F)(F)F)c2ccc(C3=CCOCC3)cc21]

A mixture of (S)-1-(6-bromo-1-neopentyl-1H-indol-3-yl)-2,2,2-trifluoroethanamine hydrochloride (370 mg, 925.75 μmol), 2-(3,6-dihydro-2H-pyran-4-yl)-4,4,5,5-tetramethyl-1,3,2-dioxaborolane (291.72 mg, 1.39 mmol), Pd(dppf)Cl2 (135.47 mg, 185.15 μmol) and Cs2CO3 (1.21 g, 3.70 mmol) in dioxane (16 mL) and water (4 mL) was stirred at 80° C. overnight under Nitrogen. The reaction mixture was cooled to room temperature, added water and ethyl acetate, stirred and separated out the organic phase. the aqueous phase was extracted with ethyl acetate twice. The combined organic phases were washed with brine, dried with anhydrous Na2SO4 and filtered. The filtrate was concentrated to give the crude product, which was purified by silica gel column (EA/PE=0-50%) to get (S)-1-(6-(3,6-dihydro-2H-pyran-4-yl)-1-neopentyl-1H-indol-3-yl)-2,2,2-trifluoroethanamine (337 mg, 919.71 μmol, 99.35% yield) as yellow oil.

### Step 2: (S)-2,2,2-trifluoro-1-(1-neopentyl-6-(tetrahydro-2H-pyran-4-yl)-1H-indol-3-yl)ethanamine

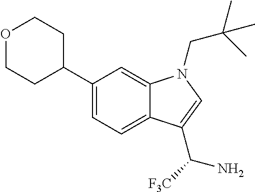 [SMILES: CC(C)(C)Cn1cc([C@H](N)C(F)(F)F)c2ccc(C3CCOCC3)cc21]

A mixture of (S)-1-(6-(3,6-dihydro-2H-pyran-4-yl)-1-neopentyl-1H-indol-3-yl)-2,2,2-trifluoroethanamine (337 mg, 919.71 μmol) and 10% Pd/C (68 mg) in methanol (10 mL) was stirred at room temperature overnight under H2. The mixture was filtered, The filtrate was concentrated to get (S)-2,2,2-trifluoro-1-(1-neopentyl-6-(tetrahydro-2H-pyran-4-yl)-1H-indol-3-yl)ethanamine (338 mg, 917.39 μmol, 99.75% yield) as yellow oil.

### Step 3: (S)—N-(2,2,2-trifluoro-1-(1-neopentyl-6-(tetrahydro-2H-pyran-4-yl)-1H-indol-3-yl)ethyl)cyclobutanesulfonamide

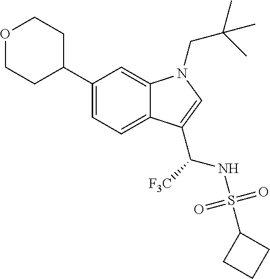 [SMILES: CC(C)(C)Cn1cc([C@H](NS(=O)(=O)C2CCC2)C(F)(F)F)c2ccc(C3CCOCC3)cc21]

A mixture of (S)-2,2,2-trifluoro-1-(1-neopentyl-6-(tetrahydro-2H-pyran-4-yl)-1H-indol-3-yl)ethanamine (338 mg, 917.39 μmol) and cyclobutanesulfonyl chloride (141.84 mg, 917.39 μmol) in anhydrous pyridine (0.7 mL) was stirred at 100° C. for 4 h in a microwave reactor. The mixture was added 1N HCl and EtOAc. The organic phase was separated, the aqueous phase was extracted with EtOAc twice, the combined organic phases were washed with brine, dried with anhydrous Na2SO4 and filtered. The filtrate was concentrated to get black oil, which was purified by prep-HPLC to get (S)—N-(2,2,2-trifluoro-1-(1-neopentyl-6-(tetrahydro-2H-pyran-4-yl)-1H-indol-3-yl)ethyl)cyclobutanesulfonamide (21.11 mg, 43.38 μmol, 4.73% yield) as a light-yellow solid. ¹H NMR (400 MHz, CDCl₃) δ 7.57 (d, J=8.2 Hz, 1H), 7.09 (d, J=18.3 Hz, 2H), 6.98 (d, J=8.2 Hz, 1H), 5.26 (s, 1H), 5.06 (s, 1H), 4.03 (d, J=9.5 Hz, 2H), 3.83-3.66 (m, 3H), 3.49 (t, J=11.0 Hz, 2H), 2.80 (t, J=11.3 Hz, 1H), 2.35 (dt, J=22.0, 8.8 Hz, 2H), 2.17-1.99 (m, 2H), 1.86-1.70 (m, 6H), 0.91 (s, 9H). LCMS: 487.2 m/z [M+1]⁺.

### Example 520: (S)—N-(1-(6-(2-cyano-6-fluorophenyl)-1-cyclobutyl-1H-indol-3-yl)-2,2,2-trifluoroethyl)cyclobutanesulfonamide (520)

### Step 1: 6-bromo-1-cyclobutyl-indole

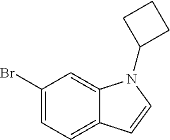 [SMILES: Brc1ccc2ccn(C3CCC3)c2c1]

A mixture of 6-bromo-1H-indole (1.5 g, 7.65 mmol), bromocyclobutane (1.24 g, 9.18 mmol) and KOH (858.57 mg, 15.30 mmol, 420.04 μL) in anhydrous DMF (10 mL) was stirred at 80° C. for 5 h, the reaction mixture was cooled to room temperature, added ice water and stirred. The aqueous phase was extracted with CH2Cl2 twice. The combined organic phases were washed with brine, dried with anhydrous Na2SO4 and filtered. The filtrate was concentrated to give brown oil, which was purified by silica gel column (PE=100%) to get 6-bromo-1-cyclobutyl-indole (1.3 g, 5.20 mmol, 67.93% yield) as yellow oil.

### Step 2: (S)—N—((S)-1-(6-bromo-1-cyclobutyl-1H-indol-3-yl)-2,2,2-trifluoroethyl)-2-methylpropane-2-sulfinamide

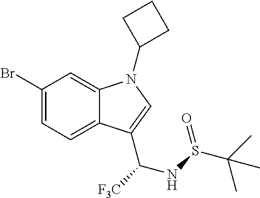 [SMILES: CC(C)(C)[S@](=O)N[C@@H](c1cn(C2CCC2)c2cc(Br)ccc12)C(F)(F)F]

To a stirred solution of 6-bromo-1-cyclobutyl-indole (1.3 g, 5.20 mmol) in anhydrous CH2Cl2 (10 mL) was added BF3·OEt2 (1.33 g, 9.35 mmol, 1.15 mL) at 0° C. under Nitrogen, followed by addition the CH2Cl2 mixture of a solution of (S,E)-2-methyl-N-(2,2,2-trifluoroethylidene)propane-2-sulfinamide in DCM (30 mL). The resulting mixture was stirred at room temperature for 2 h under Nitrogen. To the mixture was added water, stirred and separated organic phase, the aqueous phase was extracted with CH2Cl2 once. The combined organic phases were washed with brine and filtered. The filtrate was concentrated to get (S)—N—((S)-1-(6-bromo-1-cyclobutyl-1H-indol-3-yl)-2,2,2-trifluoroethyl)-2-methylpropane-2-sulfinamide as yellow oil, which was used directly next step.

### Step 3: (S)-1-(6-bromo-1-cyclobutyl-1H-indol-3-yl)-2,2,2-trifluoroethanamine

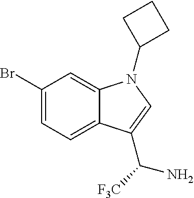 [SMILES: N[C@@H](c1cn(C2CCC2)c2cc(Br)ccc12)C(F)(F)F]

A mixture of (S)—N—((S)-1-(6-bromo-1-cyclobutyl-1H-indol-3-yl)-2,2,2-trifluoroethyl)-2-methylpropane-2-sulfinamide (2.35 g, 5.20 mmol) in 4M HCl(g)/ethanol (25 mL) was stirred at room temperature for 2 hours, the mixture was concentrated, the residue was basified to pH=8 with sat. NaHCO3, the aqueous phase was extracted with EtOAc twice, the combined organic phases were washed with brine, dried with anhydrous Na2SO4 and filtered. The filtrate was concentrated to give the crude product, which was purified by silica gel column (EA/PE=0:1 to 1:1) to get (S)-1-(6-bromo-1-cyclobutyl-1H-indol-3-yl)-2,2,2-trifluoroethanamine (1.80 g, 5.18 mmol, 99.71% yield) as yellow oil.

### Step 4: (S)-2-(3-(1-amino-2,2,2-trifluoroethyl)-1-cyclobutyl-1H-indol-6-yl)-3-fluorobenzonitrile

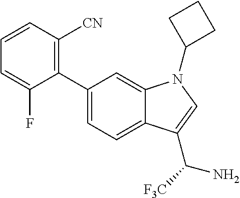 [SMILES: N#Cc1cccc(F)c1-c1ccc2c([C@H](N)C(F)(F)F)cn(C3CCC3)c2c1]

A mixture of (S)-1-(6-bromo-1-cyclobutyl-1H-indol-3-yl)-2,2,2-trifluoroethanamine (700 mg, 2.02 mmol), (2-cyano-6-fluoro-phenyl)boronic acid (498.82 mg, 3.02 mmol), Pd(dppf)Cl2 (295.06 mg, 403.26 μmol) and K2CO3 (835.99 mg, 6.05 mmol) in anhydrous DMF (6 mL) was stirred at 80° C. for 1.5 h in the microwave under Nitrogen. The reaction mixture was cooled to room temperature, added water and ethyl acetate, stirred and separated out the organic phase. The aqueous phase was extracted with ethyl acetate twice. The combined organic phases were washed with brine, dried with anhydrous Na2SO4 and filtered. The filtrate was concentrated to give the crude product, which was purified by silica gel column (EA/PE=0:1 to 1:2) to get (S)-2-(3-(1-amino-2,2,2-trifluoroethyl)-1-cyclobutyl-1H-indol-6-yl)-3-fluorobenzonitrile (100 mg, 58.03 μmol, 2.88% yield, 22.48% purity) as brown oil.

### Step 5: (S)—N-(1-(6-(2-cyano-6-fluorophenyl)-1-cyclobutyl-1H-indol-3-yl)-2,2,2-trifluoroethyl)cyclobutanesulfonamide

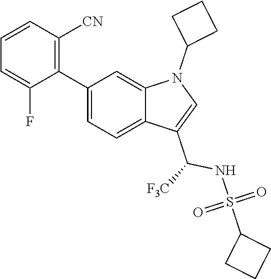 [SMILES: N#Cc1cccc(F)c1-c1ccc2c([C@H](NS(=O)(=O)C3CCC3)C(F)(F)F)cn(C3CCC3)c2c1]

A mixture of (S)-2-(3-(1-amino-2,2,2-trifluoroethyl)-1-cyclobutyl-1H-indol-6-yl)-3-fluorobenzonitrile (100 mg, 58.03 μmol, purity: 22.48%) and cyclobutanesulfonyl chloride (161.51 mg, 1.04 mmol) in anhydrous pyridine (0.3 mL) was stirred at 100° C. for 4 hours in the microwave. The mixture was added CH2Cl2 and concentrated to get black oil, which was purified by prep-HPLC to get (S)—N-(1-(6-(2-cyano-6-fluorophenyl)-1-cyclobutyl-1H-indol-3-yl)-2,2,2-trifluoroethyl)cyclobutanesulfonamide (2.77 mg, 5.48 μmol, 9.44% yield) as a light-yellow solid. ¹H NMR (400 MHz, CDCl₃) δ 7.82 (d, J=8.3 Hz, 1H), 7.63-7.58 (m, 1H), 7.52 (s, 1H), 7.46-7.38 (m, 3H), 7.31 (dt, J=8.3, 1.7 Hz, 1H), 5.45-5.35 (m, 1H), 4.92-4.83 (m, 1H), 4.74 (d, J=6.7 Hz, 1H), 3.83 (p, J=8.4 Hz, 1H), 2.66-2.57 (m, 2H), 2.55-2.42 (m, 4H), 2.30-2.18 (m, 2H), 2.02-1.89 (m, 4H). LCMS: 523.2 m/z [M+1]⁺.

### Example 521: (S)—N-(1-(6-cyclopentenyl-1-neopentyl-1H-indol-3-yl)-2,2,2-trifluoroethyl)cyclobutanesulfonamide (521)

### Step 1: (S)-1-(6-cyclopentenyl-1-neopentyl-1H-indol-3-yl)-2,2,2-trifluoroethanamine

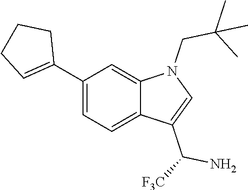 [SMILES: CC(C)(C)Cn1cc([C@H](N)C(F)(F)F)c2ccc(C3=CCCC3)cc21]

A mixture of (S)-1-(6-bromo-1-neopentyl-1H-indol-3-yl)-2,2,2-trifluoroethanamine hydrochloride (440 mg, 1.10 mmol), 2-(cyclopenten-1-yl)-4,4,5,5-tetramethyl-1,3,2-dioxaborolane (320.49 mg, 1.65 mmol), Pd(dppf)Cl2 (161.10 mg, 220.18 μmol) and Cs2CO3 (1.43 g, 4.40 mmol) in dioxane (16 mL) and water (4 mL) was stirred at 90° C. overnight under Nitrogen. The reaction mixture was concentrated to remove solvent, to the residue was added water and extracted with ethyl acetate twice. The combined organic phases were washed with brine, dried with anhydrous Na2SO4 and filtered. The filtrate was concentrated to give the crude product, which was purified by silica gel column (EA/PE=0-25%) to get (S)-1-(6-cyclopentenyl-1-neopentyl-1H-indol-3-yl)-2,2,2-trifluoroethanamine (330 mg, 941.73 μmol, 85.54% yield) as yellow oil.

### Step 2: (S)—N-(1-(6-cyclopentenyl-1-neopentyl-1H-indol-3-yl)-2,2,2-trifluoroethyl)cyclobutanesulfonamide

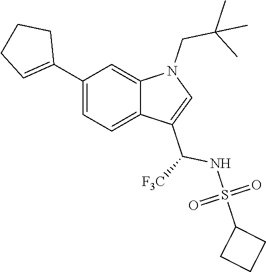 [SMILES: CC(C)(C)Cn1cc([C@H](NS(=O)(=O)C2CCC2)C(F)(F)F)c2ccc(C3=CCCC3)cc21]

A mixture of (S)-1-(6-cyclopentenyl-1-neopentyl-1H-indol-3-yl)-2,2,2-trifluoroethanamine (200 mg, 570.74 μmol) and cyclobutanesulfonyl chloride (226.92 mg, 1.03 mmol) in anhydrous pyridine (0.4 mL) was stirred at 100° C. for 4 hours in the microwave. The mixture was added EtOAc and concentrated to get black oil, which was purified by silica gel column (EA/PE: 0:1 to 1:1) to get crude. which was purified by prep-HPLC to get (S)—N-(1-(6-cyclopentenyl-1-neopentyl-1H-indol-3-yl)-2,2,2-trifluoroethyl)cyclobutanesulfonamide (32.35 mg, 69.04 μmol, 12.10% yield) as a white solid. ¹H NMR (400 MHz, CDCl₃) δ 7.61 (d, J=8.4 Hz, 1H), 7.36 (dd, J=8.4, 1.3 Hz, 1H), 7.32 (s, 1H), 7.12 (s, 1H), 6.22-6.15 (m, 1H), 5.32 (p, J=7.8 Hz, 1H), 4.92 (d, J=8.3 Hz, 1H), 3.86 (s, 2H), 3.75 (p, J=8.3 Hz, 1H), 2.82-2.73 (m, 2H), 2.56 (td, J=7.7, 2.4 Hz, 2H), 2.43 (tt, J=18.0, 9.1 Hz, 2H), 2.23-2.14 (m, 1H), 2.05 (ddd, J=21.7, 10.9, 4.5 Hz, 3H), 1.92-1.84 (m, 2H), 0.99 (s, 9H). LCMS: 469.2 m/z [M+1]⁺.

### Example 522: (S)-3-hydroxy-N-(2,2,2-trifluoro-1-(1-neopentyl-6-(2-(trifluoromethyl)phenyl)-1H-indol-3-yl)ethyl)cyclobutane-1-sulfonamide (522)

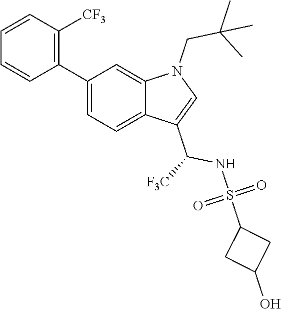 [SMILES: CC(C)(C)Cn1cc([C@H](NS(=O)(=O)C2CC(O)C2)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 3-hydroxycyclobutane-1-sulfonyl chloride. ESI-MS (m/z): 562.78 [M+1]⁺.

### Example 523: (S)—N-(2,2,2-trifluoro-1-(1-((1-(trifluoromethyl)cyclopropyl)methyl)-6-(2-(trifluoromethyl)phenyl)-1H-indol-3-yl)ethyl)cyclobutanesulfonamide (523)

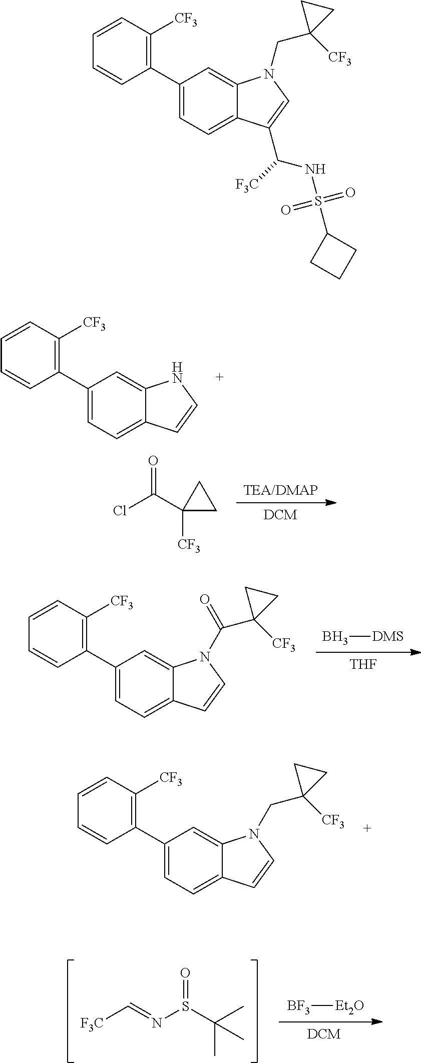

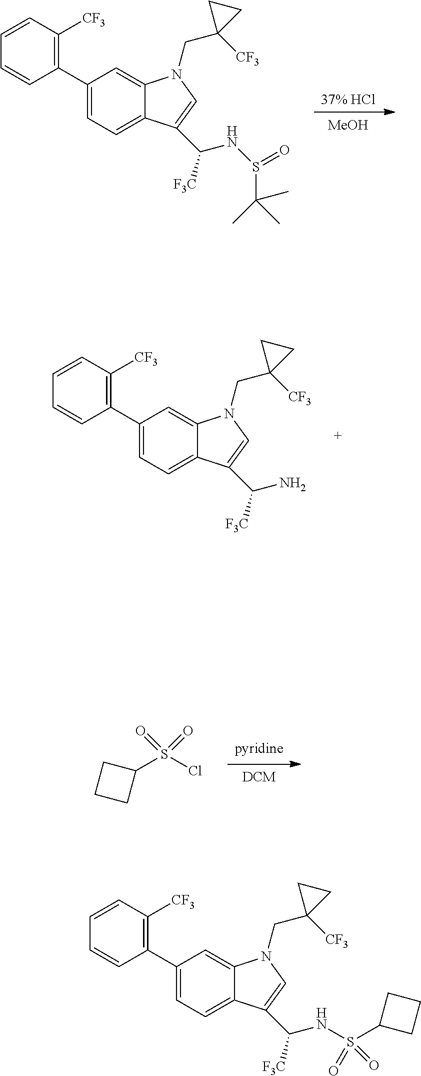

### Step 1: (1-(trifluoromethyl)cyclopropyl)(6-(2-(trifluoromethyl)phenyl)-1H-indol-1-yl)methanone

The title compound was prepared following the same general protocol as described for Example 34, using 6-(2-(trifluoromethyl)phenyl)-1H-indole and 1-(trifluoromethyl)cyclopropane-1-carbonyl chloride. ESI-MS (m/z): 398.1 [M+1]⁺.

### Step 2: 1-((1-(trifluoromethyl)cyclopropyl)methyl)-6-(2-(trifluoromethyl)phenyl)-1H-indole

The title compound was prepared following the same general protocol as described for Step 3, Example 1, using (1-(trifluoromethyl)cyclopropyl)(6-(2-(trifluoromethyl)phenyl)-1H-indol-1-yl)methanone. ESI-MS (m/z): 384.1 [M+1]⁺.

### Step 3: (S)-2-methyl-N—((S)-2,2,2-trifluoro-1-(1-((1-(trifluoromethyl)cyclopropyl)methyl)-6-(2-(trifluoromethyl)phenyl)-1H-indol-3-yl)ethyl)propane-2-sulfinamide

The title compound was prepared following the same general protocol as described for Step 3, Example 116, using 1-((1-(trifluoromethyl)cyclopropyl)methyl)-6-(2-(trifluoromethyl)phenyl)-1H-indole. ESI-MS (m/z): 585.2 [M+1]⁺.

### Step 4: (S)-2,2,2-trifluoro-1-(1-((1-(trifluoromethyl)cyclopropyl)methyl)-6-(2-(trifluoromethyl)-phenyl)-1H-indol-3-yl)ethan-1-amine

The title compound was prepared following the same general protocol as described for Step 4, Example 116, using (S)-2-methyl-N—((S)-2,2,2-trifluoro-1-(1-((1-(trifluoromethyl)cyclopropyl)-methyl)-6-(2-(trifluoromethyl)phenyl)-1H-indol-3-yl)ethyl)propane-2-sulfinamide. ESI-MS (m/z): 464.1 [M-NH₂]⁺.

### Step 5: (S)—N-(2,2,2-trifluoro-1-(1-((1-(trifluoromethyl)cyclopropyl)methyl)-6-(2-(trifluoromethyl)phenyl)-1H-indol-3-yl)ethyl)cyclobutanesulfonamide

The title compound was prepared following the same general protocol as described for Step 5, Example 116, using (S)-2,2,2-trifluoro-1-(1-((1-(trifluoromethyl)cyclopropyl)methyl)-6-(2-(trifluoromethyl)phenyl)-1H-indol-3-yl)ethan-1-amine and cyclobutanesulfonyl chloride. ESI-MS (m/z): 599.1 [M+1]⁺.

### Example 524: (S)—N-(2,2,2-trifluoro-1-(5-fluoro-1-neopentyl-6-(2-(trifluoromethyl)phenyl)-1H-pyrrolo[2,3-b]pyridin-3-yl)ethyl)cyclopropanesulfonamide (524)

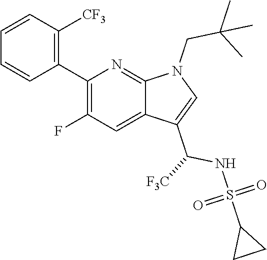 [SMILES: CC(C)(C)Cn1cc([C@H](NS(=O)(=O)C2CC2)C(F)(F)F)c2cc(F)c(-c3ccccc3C(F)(F)F)nc21]

### Step 1: 5-fluoro-1H-pyrrolo[2,3-b]pyridine 7-oxide

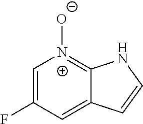 [SMILES: [O-][n+]1cc(F)cc2cc[nH]c21]

To the mixture of 5-fluoro-1H-pyrrolo[2,3-b]pyridine (5.04 g, 37.03 mmol) in ether (200 mL) was added mCPBA (12.8 g, 55.54 mmol) portionwise. The mixture was stirred at rt overnight. The mixture was diluted with ether and washed with saturated NaHCO₃ and brine, dried over Na₂SO₄. The solvent was removed to obtain the title compound with no further purification.

### Step 2: 1-(6-chloro-5-fluoro-1H-pyrrolo[2,3-b]pyridin-1-yl)-2,2-dimethylpropan-1-one

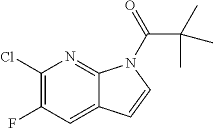 [SMILES: CC(C)(C)C(=O)n1ccc2cc(F)c(Cl)nc21]

To a solution of 5-fluoro-1H-pyrrolo[2,3-b]pyridine 7-oxide (5.255 g, 34.54 mmol) and HMDS (7.92 mL, 37.99 mmol) in THF at 0° C. under argon was added pivaloyl chloride (10.62 mL, 86.35 mml) dropwise. The mixture was stirred at rt overnight. The mixture was diluted with EtOAc and washed with saturated NaHCO₃ and brine, dried over Na₂SO₄. The solvent was removed and the crude was purified by silica gel to obtain the title compound and 6-chloro-5-fluoro-1H-pyrrolo[2,3-b]pyridine. ¹HNMR (CDCl₃, 400 MHz) δ 7.99 (d, J=4.0 Hz, 1H), 7.62 (d, J=8.0 Hz, 1H), 7.55 (d, J=8.0 Hz, 1H), 1.59 (s, 9H)

### Step 3: 6-chloro-5-fluoro-1-neopentyl-1H-pyrrolo[2,3-b]pyridine

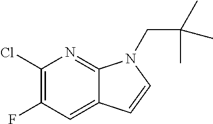 [SMILES: CC(C)(C)Cn1ccc2cc(F)c(Cl)nc21]

To a solution of 1-(6-chloro-5-fluoro-1H-pyrrolo[2,3-b]pyridin-1-yl)-2,2-dimethylpropan-1-one (3.12 g, 12.26 mmol) in THF (30 mL0 was added BH₃·DMS (3.5 mL, 36.78 mmol) slowly. The mixture was warmed to reflux overnight. The reaction was monitored by anal. HPLC for completion. The mixture was cooled to 0° C. and water was added slowly to quench the reaction. The mixture was diluted with EtOAc and washed with saturated NaHCO₃ and brine, dried over Na₂SO₄. The solvent was removed and the crude was purified by silica gel to obtain the title compound. ¹HNMR (CDCl₃, 400 MHz) δ 7.64 (d, J=8.0 Hz, 1H), 7.23 (d, J=4.0 Hz, 1H), 6.41 (d, J=4.0 Hz, 1H), 4.05 (s, 2H), 0.98 (s, 9H)

### Step 4: (R)—N—((S)-1-(6-chloro-5-fluoro-1-neopentyl-1H-pyrrolo[2,3-b]pyridin-3-yl)-2,2,2-trifluoroethyl)-2-methylpropane-2-sulfinamide

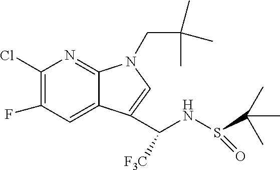 [SMILES: CC(C)(C)Cn1cc([C@H](N[S@](=O)C(C)(C)C)C(F)(F)F)c2cc(F)c(Cl)nc21]

The title compound was prepared following the same general protocol as described for Step 3, Example 116, using (S)-2-methyl-N-(3,3,3-trifluoroprop-1-en-1-yl)propane-2-sulfinamide and 6-chloro-5-fluoro-1-neopentyl-1H-pyrrolo[2,3-b]pyridine. ¹HNMR (CDCl₃, 400 MHz) δ 7.91 (d, J=8.0 Hz, 1H), 7.44 (s, 1H), 4.68 (q, J=8.0 Hz, 1H), 4.14-4.00 (m, 3H), 1.22 (s, 6H), 1.20 (s, 3H), 0.98 (s, 9H)

### Step 5: (S)-1-(6-chloro-5-fluoro-1-neopentyl-1H-pyrrolo[2,3-b]pyridin-3-yl)-2,2,2-trifluoroethan-1-amine

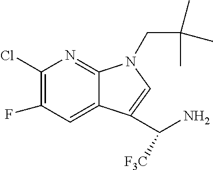 [SMILES: CC(C)(C)Cn1cc([C@H](N)C(F)(F)F)c2cc(F)c(Cl)nc21]

The title compound was prepared following the same general protocol as described for Step 4, Example 116, using 2-methyl-N—((S)-2,2,2-trifluoro-1-(6-(4-fluoro-2-(trifluoromethyl)phenyl)-1-neopentyl-1H-indol-3-yl)ethyl)propane-2-sulfinamide.

### Step 6: (S)-2,2,2-trifluoro-1-(5-fluoro-1-neopentyl-6-(2-(trifluoromethyl)phenyl)-1H-pyrrolo[2,3-b]pyridin-3-yl)ethan-1-amine

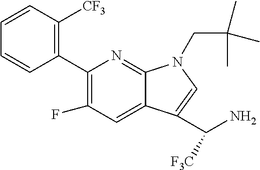 [SMILES: CC(C)(C)Cn1cc([C@H](N)C(F)(F)F)c2cc(F)c(-c3ccccc3C(F)(F)F)nc21]

The title compound was prepared following the same general protocol as described for Step 5, Example 1, using (S)-1-(6-chloro-5-fluoro-1-neopentyl-1H-pyrrolo[2,3-b]pyridin-3-yl)-2,2,2-trifluoroethan-1-amine and (2-(trifluoromethyl)phenyl)boronic acid. ¹HNMR (CDCl₃, 400 MHz) δ 7.91-7.80 (m, 2H), 7.64 (t, J=8.0 Hz, 1H), 7.56 (t, J=8.0 Hz, 1H), 7.46 (d, J=4.0 Hz, 1H), 7.40 (s, 1H), 4.72 (q, J=8.0 Hz, 1H), 4.09 (d, J=4.0 Hz, 2H), 0.96 (s, 9H)

### Step 7: (S)—N-(2,2,2-trifluoro-1-(5-fluoro-1-neopentyl-6-(2-(trifluoromethyl)phenyl)-1H-pyrrolo[2,3-b]pyridin-3-yl)ethyl)cyclopropanesulfonamide

The title compound was prepared following the same general protocol as described for Step 5, Example 116, using (S)-2,2,2-trifluoro-1-(5-fluoro-1-neopentyl-6-(2-(trifluoromethyl)phenyl)-1H-pyrrolo[2,3-b]pyridin-3-yl)ethan-1-amine and cyclopropanesulfonyl chloride. HNMR (CDCl₃, 400 MHz) δ 7.84 (d, J=8.0 Hz, 1H), 7.80 (d, J=8.0 Hz, 1H), 7.64 (t, J=8.0 Hz, 2H), 7.57 (t, J=8.0 Hz, 1H), 7.44 (d, J=8.0 Hz, 1H), 7.42 (s, 1H), 5.37 (q, J=8.0 Hz, 1H), 4.97 (d, J=8.0 Hz, 1H), 4.09 (s, 2H), 2.49-2.43 (m, 1H), 1.28-1.23 (m, 1H), 1.16-1.09 (m, 1H), 1.08-0.93 (m, 11H)

### Example 525: (S)—N-(2,2,2-trifluoro-1-(5-fluoro-1-neopentyl-6-(2-(trifluoromethyl)phenyl)-1H-pyrrolo[2,3-b]pyridin-3-yl)ethyl)cyclobutanesulfonamide (525)

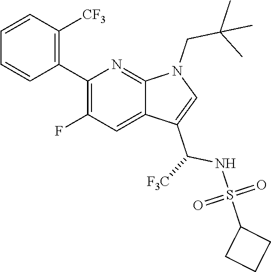 [SMILES: CC(C)(C)Cn1cc([C@H](NS(=O)(=O)C2CCC2)C(F)(F)F)c2cc(F)c(-c3ccccc3C(F)(F)F)nc21]

The title compound was prepared following the same general protocol as described for Step 5, Example 116, using (S)-2,2,2-trifluoro-1-(5-fluoro-1-neopentyl-6-(2-(trifluoromethyl)phenyl)-1H-pyrrolo[2,3-b]pyridin-3-yl)ethan-1-amine and cyclobutanesulfonyl chloride. ¹HNMR (CDCl₃, 400 MHz) δ 7.83 (d, J=8.0 Hz, 1H), 7.78 (d, J=8.0 Hz, 1H), 7.64 (t, J=8.0 Hz, 2H), 7.57 (t, J=8.0 Hz, 1H), 7.44 (d, J=8.0 Hz, 1H), 7.40 (s, 1H), 5.33 (q, J=8.0 Hz, 1H), 4.84 (d, J=8.0 Hz, 1H), 4.11 (s, 2H), 3.86-3.78 (m, 1H), 2.67-2.44 (m, 2H), 2.29-2.20 (m, 2H), 2.04-1.92 (m, 2H), 0.95 (s, 9H)

### Example 526: Step 3: (S)—N-(1-(6-(2-cyano-6-fluorophenyl)-1-(tetrahydro-2H-pyran-4-yl)-1H-indol-3-yl)-2,2,2-trifluoroethyl)cyclobutanesulfonamide (526)

### Step 1: (S)-2,2,2-trifluoro-1-(1-(tetrahydro-2H-pyran-4-yl)-6-(4,4,5,5-tetramethyl-1,3,2-dioxaborolan-2-yl)-1H-indol-3-yl)ethan-1-amine

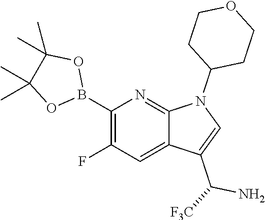 [SMILES: CC1(C)OB(c2nc3c(cc2F)c([C@H](N)C(F)(F)F)cn3C2CCOCC2)OC1(C)C]

A mixture of (1S)-1-(6-bromo-1-tetrahydropyran-4-yl-indol-3-yl)-2,2,2-trifluoro-ethanamine (600 mg, 731.71 μmol) in dioxane (10 mL) was added Pd(dppf)Cl₂ (53.56 mg, 73.17 μmol) and KOAc (215.12 mg, 2.20 mmol). The mixture was purged by N2 for 30 seconds. The mixture was stirred at 90° C. for 3 hours. The mixture was filtered and the filtration was concentrated in vacuo. A crude product (460 mg) was obtained.

### Step 2: (S)-2-(3-(1-amino-2,2,2-trifluoroethyl)-1-(tetrahydro-2H-pyran-4-yl)-1H-indol-6-yl)-3-fluorobenzonitrile

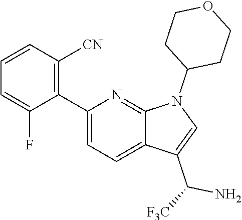 [SMILES: N#Cc1cccc(F)c1-c1ccc2c([C@H](N)C(F)(F)F)cn(C3CCOCC3)c2n1]

A mixture of (1S)-2,2,2-trifluoro-1-[l-tetrahydropyran-4-yl-6-(4,4,5,5-tetramethyl-1,3,2-dioxaborolan-2-yl)indol-3-yl]ethanamine (310 mg, 584.54 μmol) and 2-bromo-3-fluoro-benzonitrile (140.30 mg, 701.45 μmol) in dioxane (8 mL) and H₂O (2 mL) was added Pd(dppf)Cl₂ (42.79 mg, 58.45 μmol) and K₂CO₃ (161.33 mg, 1.17 mmol). The mixture was purged by N₂ for 10 seconds. The mixture was stirred at 90° C. for 5 hours. The mixture was extracted with DCM (20 mL×3) and H₂O (10 mL). The organic layers were combined and washed by brine (10 mL). The organic layers were concentrated in vacuo and the product was purified by chromatograph silica on gel (PE:EA, 0%˜50%, UV=254 nm). 3-fluoro-2-[3-[rac-(1S)-1-amino-2,2,2-trifluoro-ethyl]-1-tetrahydropyran-4-yl-indol-6-yl]benzonitrile (130 mg, 264.73 μmol, 45.29% yield, 85% purity) as a yellow solid was obtained.

### Step 3: (S)—N-(1-(6-(2-cyano-6-fluorophenyl)-1-(tetrahydro-2H-pyran-4-yl)-1H-indol-3-yl)-2,2,2-trifluoroethyl)cyclobutanesulfonamide

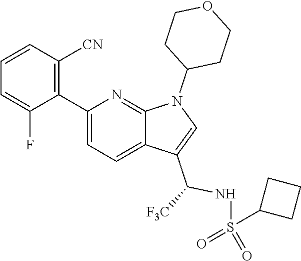 [SMILES: N#Cc1cccc(F)c1-c1ccc2c([C@H](NS(=O)(=O)C3CCC3)C(F)(F)F)cn(C3CCOCC3)c2n1]

A mixture of 3-fluoro-2-[3-[(1S)-1-amino-2,2,2-trifluoro-ethyl]-1-tetrahydropyran-4-yl-indol-6-yl]benzonitrile (120 mg, 287.49 μmol) and cyclobutanesulfonyl chloride (66.68 mg, 431.24 μmol) in Pyridine (2 mL) was stirred at 100° C. under microwave for 4 hours. The mixture was concentrated in vacuo and purified by chromatograph silica on gel (EA:PE, 0%˜60%, UV=254 nm). A crude product (80 mg) was obtained. The product was purified by Prep-HPLC (base) to give N-[(1S)-1-[6-(2-cyano-6-fluoro-phenyl)-1-tetrahydropyran-4-yl-indol-3-yl]-2,2,2-trifluoro-ethyl]cyclobutanesulfonamide (8 mg, 14.94 μmol, 5.20% yield) as a white solid. 1H NMR (400 MHz, CDCl3) δ 7.85 (d, J=8.3 Hz, 1H), 7.63-7.58 (m, 1H), 7.56 (s, 1H), 7.43 (dd, J=13.0, 6.5 Hz, 3H), 7.32 (d, J=8.3 Hz, 1H), 5.40 (dd, J=15.4, 7.7 Hz, 1H), 5.07 (d, J=8.1 Hz, 1H), 4.46 (dd, J=18.6, 13.0 Hz, 1H), 4.19-4.05 (m, 2H), 3.82 (dd, J=16.6, 8.3 Hz, 1H), 3.58 (ddd, J=14.1, 11.7, 6.5 Hz, 2H), 2.48 (dq, J=18.1, 9.1 Hz, 2H), 2.23 (dd, J=18.9, 12.1 Hz, 2H), 2.12-1.91 (m, 6H). MS Found: 552.7 [M+H₂O]⁺.

### Example 527: (S)—N-(2,2-difluoro-1-(6-(2-fluoro-6-(trifluoromethyl)phenyl)-1-neopentyl-1H-indol-3-yl)ethyl)cyclobutanesulfonamide (527)

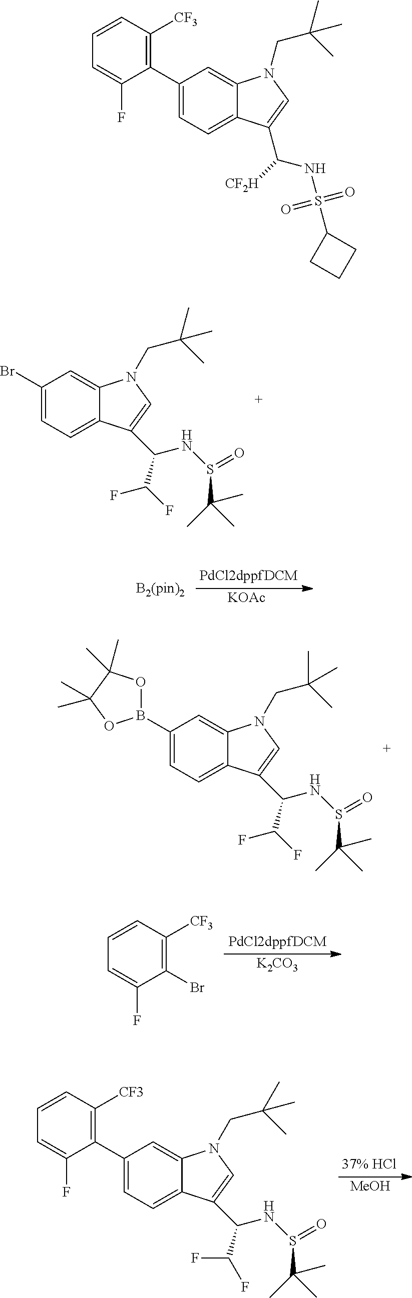

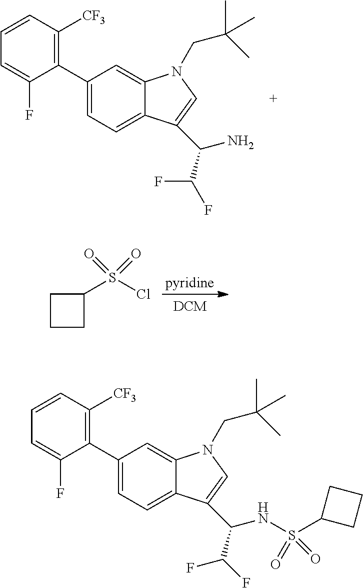

### Step 1: (S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide

The title compound was prepared following the same general protocol as described for Step 2, Example 33, using (S)—N—((S)-1-(6-bromo-1-neopentyl-1H-indol-3-yl)-2,2-difluoroethyl)-2-methylpropane-2-sulfinamide. ESI-MS (m/z): 512.3 [M+1]⁺.

### Step 2: (S)—N—((S)-2,2-difluoro-1-(6-(2-fluoro-6-(trifluoromethyl)phenyl)-1-neopentyl-1H-indol-3-yl)ethyl)-2-methylpropane-2-sulfinamide

The title compound was prepared following the same general protocol as described for Step 5, Example 1, using (S)—N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide and 2-bromo-1-fluoro-3-(trifluoromethyl)benzene. ESI-MS (m/z): 533.2 [M+1]⁺.

### Step 3: (S)-2,2-difluoro-1-(6-(2-fluoro-6-(trifluoromethyl)phenyl)-1-neopentyl-1H-indol-3-yl)ethan-1-amine

The title compound was prepared following the same general protocol as described for Step 4, Example 116, using (S)—N—((S)-2,2-difluoro-1-(6-(2-fluoro-6-(trifluoromethyl)phenyl)-1-neopentyl-1H-indol-3-yl)ethyl)-2-methylpropane-2-sulfinamide. ESI-MS (m/z): 412.2 [M-NH₂]⁺.

### Step 4: (S)—N-(2,2-difluoro-1-(6-(2-fluoro-6-(trifluoromethyl)phenyl)-1-neopentyl-1H-indol-3-yl)ethyl)cyclobutanesulfonamide

The title compound was prepared following the same general protocol as described for Step 5, Example 116, using (S)-2,2-difluoro-1-(6-(2-fluoro-6-(trifluoromethyl)phenyl)-1-neopentyl-1H-indol-3-yl)ethan-1-amine and cyclobutanesulfonyl chloride. ESI-MS (m/z): 547.2 [M+1]⁺.

### Example 528: (S)—N-(2,2,2-trifluoro-1-(5-fluoro-1-neopentyl-6-(2-(trifluoromethyl)pyridin-3-yl)-1H-indol-3-yl)ethyl)cyclobutanesulfonamide (528)

### Step 1: (S)-2,2,2-trifluoro-1-(5-fluoro-1-neopentyl-6-(2-(trifluoromethyl)pyridin-3-yl)-1H-indol-3-yl)ethan-1-amine

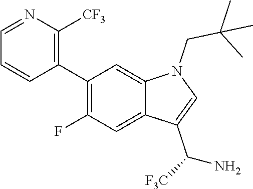 [SMILES: CC(C)(C)Cn1cc([C@H](N)C(F)(F)F)c2cc(F)c(-c3cccnc3C(F)(F)F)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 3-bromo-2-(trifluoromethyl)pyridine (172.87 mg, 764.94 μmol) in dioxane (10 mL) and H2O (2 mL) was added Pd(dppf)Cl2 (43.07 mg, 58.84 μmol) and K2CO3 (162.40 mg, 1.18 mmol). The mixture was purged by N2 for 10 seconds. The mixture was stirred at 85° C. for 5 hours. The mixture was combined with another lot. The product was purified by chromatograph silica on gel (EA:PE, 0%˜60%, UV=254 nm). rac-(1S)-1-[1-(2,2-dimethylpropyl)-5-fluoro-6-[2-(trifluoromethyl)-3-pyridyl]indol-3-yl]-2,2,2-trifluoro-ethanamine (250 mg, 558.79 μmol, 94.97% yield) as a yellow gum was obtained.

### Step 2: (S)—N-(2,2,2-trifluoro-1-(5-fluoro-1-neopentyl-6-(2-(trifluoromethyl)pyridin-3-yl)-1H-indol-3-yl)ethyl)cyclobutanesulfonamide

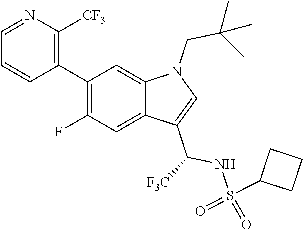 [SMILES: CC(C)(C)Cn1cc([C@H](NS(=O)(=O)C2CCC2)C(F)(F)F)c2cc(F)c(-c3cccnc3C(F)(F)F)cc21]

A mixture of rac-(1S)-1-[1-(2,2-dimethylpropyl)-5-fluoro-6-[2-(trifluoromethyl)-3-pyridyl]indol-3-yl]-2,2,2-trifluoro-ethanamine (250 mg, 558.79 μmol) in Pyridine (2 mL) was added cyclobutanesulfonyl chloride (250 mg, 1.62 mmol). The mixture was purged by N2 for 20 seconds. The mixture was stirred at 100° C. for 4 hours under microwave and N2. The mixture was concentrated in vacuo and the product was purified by Prep HPLC. N-[rac-(1S)-1-[1-(2,2-dimethylpropyl)-5-fluoro-6-[2-(trifluoromethyl)-3-pyridyl]indol-3-yl]-2,2,2-trifluoro-ethyl]cyclobutanesulfonamide (22 mg, 38.90 μmol, 6.96% yield) as a light yellow solid was obtained. 1H NMR (400 MHz, CDCl3) δ 8.75 (d, J=3.7 Hz, 1H), 7.77 (d, J=7.6 Hz, 1H), 7.55 (dd, J=7.8, 4.7 Hz, 1H), 7.50 (d, J=10.0 Hz, 1H), 7.28 (s, 1H), 7.21 (d, J=5.8 Hz, 1H), 5.39-5.27 (m, 1H), 5.03 (d, J=8.2 Hz, 1H), 3.84 (d, J=2.1 Hz, 3H), 2.48 (ddd, J=18.4, 13.5, 9.2 Hz, 2H), 2.22 (dd, J=13.2, 6.6 Hz, 2H), 1.99-1.89 (m, 2H), 0.97 (s, 9H). MS Found: 566.2[M+H]⁺.

### Example 529: (S)—N-(1-(6-(2-cyanopyridin-3-yl)-5-fluoro-1-neopentyl-1H-indol-3-yl)-2,2,2-trifluoroethyl)cyclobutanesulfonamide (529)

### Step 1: (S)-2,2,2-trifluoro-1-(5-fluoro-1-neopentyl-6-(4,4,5,5-tetramethyl-1,3,2-dioxaborolan-2-yl)-1H-indol-3-yl)ethan-1-amine

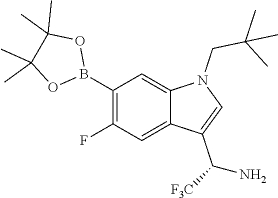 [SMILES: CC(C)(C)Cn1cc([C@H](N)C(F)(F)F)c2cc(F)c(B3OC(C)(C)C(C)(C)O3)cc21]

A mixture of rac-(1S)-1-[6-bromo-1-(2,2-dimethylpropyl)-5-fluoro-indol-3-yl]-2,2,2-trifluoro-ethanamine (3.0 g, 7.87 mmol) in dioxane (10 mL) was added Pd(dppf)Cl2 (576.07 mg, 786.98 μmol) and KOAc (2.32 g, 23.63 mmol). The mixture was purged by N2 for 30 seconds. The mixture was stirred at 90° C. for 3 hours. The mixture was concentrated in vacuo and the product was purified by chromatograph silica on gel (EA:PE, 0%˜30%, UV=254 nm). rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (2.5 g, 4.09 mmol, 51.92% yield, 70% purity) as a yellow gum was obtained.

### Step 2: (S)-3-(3-(1-amino-2,2,2-trifluoroethyl)-5-fluoro-1-neopentyl-1H-indol-6-yl)picolinonitrile

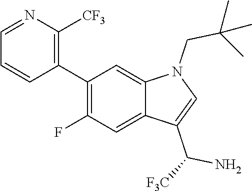 [SMILES: CC(C)(C)Cn1cc([C@H](N)C(F)(F)F)c2cc(F)c(-c3cccnc3C(F)(F)F)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 3-bromopyridine-2-carbonitrile (139.99 mg, 764.94 μmol) in dioxane (10 mL) and H2O (2 mL) was added Pd(dppf)Cl2 (43.07 mg, 58.84 μmol) and K2CO3 (162.40 mg, 1.18 mmol). The mixture was purged by N2 for 10 seconds. The mixture was stirred at 85° C. for 5 hours. The mixture was combined with another lot. The product was purified by chromatograph silica on gel (EA:PE, 0-60%, UV=254 nm). 3-[1-(2,2-dimethylpropyl)-5-fluoro-3-[rac-(1S)-1-amino-2,2,2-trifluoro-ethyl]indol-6-yl]pyridine-2-carbonitrile (230 mg, 568.74 μmol, 96.66% yield) as a yellow gum was obtained.

### Step 3: (S)—N-(1-(6-(2-cyanopyridin-3-yl)-5-fluoro-1-neopentyl-1H-indol-3-yl)-2,2,2-trifluoroethyl)cyclobutanesulfonamide

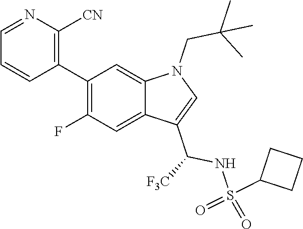 [SMILES: CC(C)(C)Cn1cc([C@H](NS(=O)(=O)C2CCC2)C(F)(F)F)c2cc(F)c(-c3cccnc3C#N)cc21]

A mixture of 3-[1-(2,2-dimethylpropyl)-5-fluoro-3-[rac-(1S)-1-amino-2,2,2-trifluoro-ethyl]indol-6-yl]pyridine-2-carbonitrile (230 mg, 568.74 μmol) and cyclobutanesulfonyl chloride (230 mg, 1.49 mmol) in pyridine (2 mL) was added pyridine (2 mL). The mixture was purged by N2 for 20 seconds. The mixture was stirred at 100° C. for 4 hours under microwave. The mixture was concentrated in vacuo. The crude product was purified by Prep HPLC. N-[rac-(1S)-1-[6-(2-cyano-3-pyridyl)-1-(2,2-dimethylpropyl)-5-fluoro-indol-3-yl]-2,2,2-trifluoro-ethyl]cyclobutanesulfonamide (8 mg, 15.31 μmol, 2.69% yield) as a light yellow solid was obtained. 1H NMR (400 MHz, CDCl3) δ 8.71 (dd, J=4.7, 1.5 Hz, 1H), 7.94-7.88 (m, 1H), 7.62-7.54 (m, 2H), 7.42 (d, J=6.0 Hz, 1H), 7.32 (s, 1H), 5.39-5.30 (m, 1H), 5.14 (d, J=8.5 Hz, 1H), 3.91-3.81 (m, 3H), 2.49 (dd, J=19.3, 9.0 Hz, 2H), 2.27 (ddd, J=9.7, 6.0, 3.2 Hz, 2H), 1.97 (td, J=9.2, 4.2 Hz, 2H), 0.99 (s, 9H). MS Found: 523.2[M+H]⁺.

### Example 530: (S)—N-(2,2,2-trifluoro-1-(5-fluoro-1-neopentyl-6-(4-(trifluoromethyl)pyridin-3-yl)-1H-indol-3-yl)ethyl)cyclobutanesulfonamide (530)

### Step 1: (S)-2,2,2-trifluoro-1-(5-fluoro-1-neopentyl-6-(4-(trifluoromethyl)pyridin-3-yl)-1H-indol-3-yl)ethan-1-amine

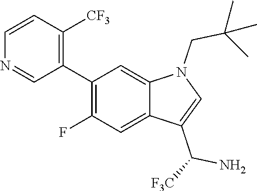 [SMILES: CC(C)(C)Cn1cc([C@H](N)C(F)(F)F)c2cc(F)c(-c3cnccc3C(F)(F)F)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 3-bromo-4-(trifluoromethyl)pyridine (172.87 mg, 764.94 μmol) in dioxane (10 mL) and H2O (2 mL) was added Pd(dppf)Cl2 (43.07 mg, 58.84 μmol) and K2CO3 (162.40 mg, 1.18 mmol). The mixture was purged by N2 for 10 seconds. The mixture was stirred at 85° C. for 5 hours. The mixture was combined with crude material from a previous test reaction using the same condition. The product was purified by chromatograph silica on gel (EA:PE, 0%˜60%, UV=254 nm). rac-(1S)-1-[1-(2,2-Dimethylpropyl)-5-fluoro-6-[4-(trifluoromethyl)-3-pyridyl]indol-3-yl]-2,2,2-trifluoro-ethanamine (250 mg, 558.79 μmol, 94.97% yield) as a yellow gum was obtained.

### Step 2: (S)—N-(2,2,2-trifluoro-1-(5-fluoro-1-neopentyl-6-(4-(trifluoromethyl)pyridin-3-yl)-1H-indol-3-yl)ethyl)cyclobutanesulfonamide

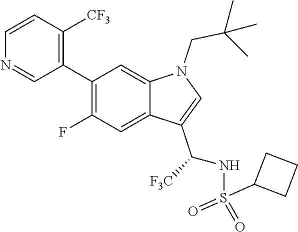 [SMILES: CC(C)(C)Cn1cc([C@H](NS(=O)(=O)C2CCC2)C(F)(F)F)c2cc(F)c(-c3cnccc3C(F)(F)F)cc21]

A mixture of rac-(1S)-1-[1-(2,2-dimethylpropyl)-5-fluoro-6-[4-(trifluoromethyl)-3-pyridyl]indol-3-yl]-2,2,2-trifluoro-ethanamine (250 mg, 558.79 μmol) in pyridine (2 mL) was added cyclobutanesulfonyl chloride (250 mg, 1.62 mmol). The mixture was purged by N2 for 20 seconds. The mixture was stirred at 100° C. for 4 hours under microwave and N2. The mixture was concentrated in vacuo and the product was purified by Prep HPLC. N-[rac-(1S)-1-[1-(2,2-dimethylpropyl)-5-fluoro-6-[4-(trifluoromethyl)-3-pyridyl]indol-3-yl]-2,2,2-trifluoro-ethyl]cyclobutanesulfonamide (33 mg, 58.35 μmol, 10.44% yield) as a light yellow solid was obtained.

¹H NMR (400 MHz, CDCl3) δ 8.82 (d, J=5.1 Hz, 1H), 8.67 (s, 1H), 7.66 (d, J=5.2 Hz, 1H), 7.51 (d, J=9.9 Hz, 1H), 7.27 (s, 1H), 7.22 (d, J=5.8 Hz, 1H), 5.34 (dd, J=15.6, 7.8 Hz, 1H), 4.97 (d, J=10.1 Hz, 1H), 3.85 (d, J=14.0 Hz, 3H), 2.49 (dt, J=20.8, 9.6 Hz, 2H), 2.34-2.16 (m, 2H), 2.05-1.85 (m, 2H), 0.97 (s, 9H). MS Found: 566.2[M+H]⁺.

### Example 533: (S)—N-(2,2-difluoro-1-(5-fluoro-1-neopentyl-6-(2-(trifluoromethyl)phenyl)-1H-pyrrolo[2,3-b]pyridin-3-yl)ethyl)cyclobutanesulfonamide (533)

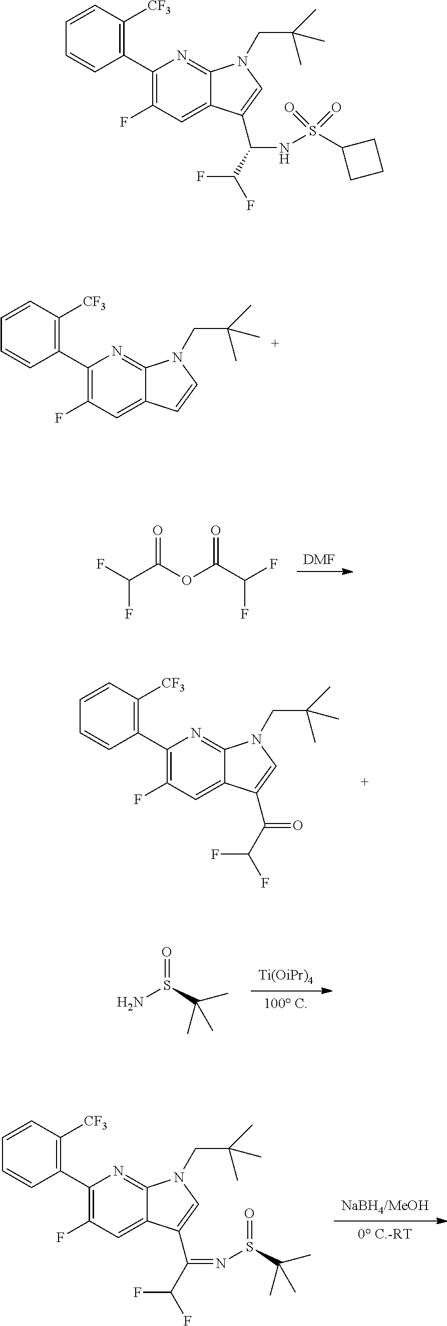

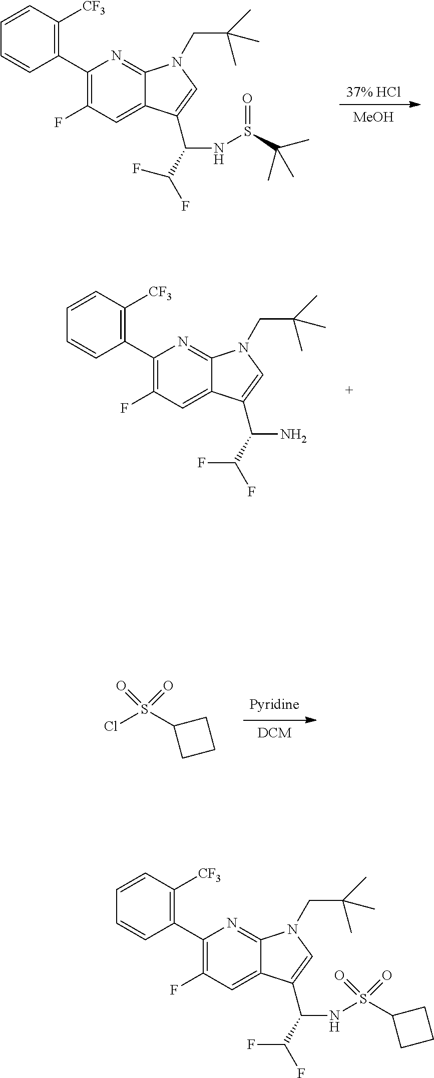

### Step 1: 2,2-difluoro-1-(5-fluoro-1-neopentyl-6-(2-(trifluoromethyl)phenyl)-1H-pyrrolo[2,3-b]pyridin-3-yl)ethan-1-one

The title compound was prepared following the same general protocol as described for Step 1, Example 128, using 5-fluoro-1-neopentyl-6-(2-(trifluoromethyl)phenyl)-1H-pyrrolo[2,3-b]pyridine and 2,2-difluoroacetic anhydride. ESI-MS (m/z): 429.1 [M+1]⁺.

### Step 2: (S,E)-N-(2,2-difluoro-1-(5-fluoro-1-neopentyl-6-(2-(trifluoromethyl)phenyl)-1H-pyrrolo[2,3-b]pyridin-3-yl)ethylidene)-2-methylpropane-2-sulfinamide

The title compound was prepared following the same general protocol as described for Step 4, Example 113, using 2,2-difluoro-1-(5-fluoro-1-neopentyl-6-(2-(trifluoromethyl)phenyl)-1H-pyrrolo[2,3-b]pyridin-3-yl)ethan-1-one. ESI-MS (m/z): 532.2 [M+1]⁺.

### Step 3: (S)—N—((S)-2,2-difluoro-1-(5-fluoro-1-neopentyl-6-(2-(trifluoromethyl)phenyl)-1H-pyrrolo[2,3-b]pyridin-3-yl)ethyl)-2-methylpropane-2-sulfinamide

The title compound was prepared following the same general protocol as described for Step 3, Example 420 (Compound 420), using (S,E)-N-(2,2-difluoro-1-(5-fluoro-1-neopentyl-6-(2-(trifluoromethyl)phenyl)-1H-pyrrolo[2,3-b]pyridin-3-yl)ethylidene)-2-methylpropane-2-sulfinamide. ESI-MS (m/z): 534.2 [M+1]⁺.

### Step 4: (S)-2,2-difluoro-1-(5-fluoro 1-neopentyl-6-(2-(trifluoromethyl)phenyl)-1H-pyrrolo[2,3-b]pyridin-3-yl)ethan-1-amine

The title compound was prepared following the same general protocol as described for Step 4, Example 116, using (S)—N—((S)-2,2-difluoro-1-(5-fluoro-1-neopentyl-6-(2-(trifluoromethyl)phenyl)-1H-pyrrolo[2,3-b]pyridin-3-yl)ethyl)-2-methylpropane-2-sulfinamide. ESI-MS (m/z): 413.2 [M-NH₂]⁺.

### Step 5: (S)—N-(2,2-difluoro-1-(5-fluoro-1-neopentyl-6-(2-(trifluoromethyl)phenyl)-1H-pyrrolo[2,3-b]pyridin-3-yl)ethyl)cyclobutanesulfonamide

The title compound was prepared following the same general protocol as described for Step 5, Example 116, using (S)-2,2-difluoro-1-(5-fluoro-1-neopentyl-6-(2-(trifluoromethyl)phenyl)-1H-pyrrolo[2,3-b]pyridin-3-yl)ethan-1-amine and cyclobutanesulfonyl chloride. ESI-MS (m/z): 548.2 [M+1]⁺.

### Example 534: (S)—N-(1-(6-(2-cyanophenyl)-1-neopentyl-1H-pyrrolo[2,3-b]pyridin-3-yl)-2,2,2-trifluoroethyl)cyclobutanesulfonamide (534)

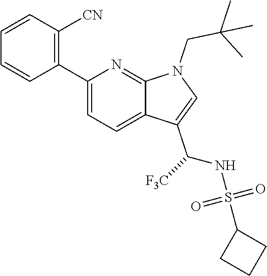 [SMILES: CC(C)(C)Cn1cc([C@H](NS(=O)(=O)C2CCC2)C(F)(F)F)c2ccc(-c3ccccc3C#N)nc21]

### Step 1: (S)-2-(3-(1-amino-2,2,2-trifluoroethyl)-1-neopentyl-1H-pyrrolo[2,3-b]pyridin-6-yl)benzonitrile

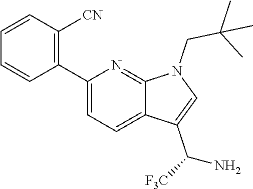 [SMILES: CC(C)(C)Cn1cc([C@H](N)C(F)(F)F)c2ccc(-c3ccccc3C#N)nc21]

The title compound was prepared following the same general protocol as described for Step 4, Example 491, using (1S)-1-[6-bromo-1-(2,2-dimethylpropyl)pyrrolo[2,3-b]pyridin-3-yl]-2,2,2-trifluoro-ethanamine and (2-cyanophenyl)boronic acid.

### Step 2: (S)—N-(1-(6-(2-cyanophenyl)-1-neopentyl-1H-pyrrolo[2,3-b]pyridin-3-yl)-2,2,2-trifluoroethyl)cyclobutanesulfonamide

The title compound was prepared following the same general protocol as described for Step 5, Example 491, using (S)-2-(3-(1-amino-2,2,2-trifluoroethyl)-1-neopentyl-1H-pyrrolo[2,3-b]pyridin-6-yl)benzonitrile and cyclobutanesulfonyl chloride. ESI-MS (m/z): 505.2 [M+1]⁺.

### Example 535: (S)—N-(1-(6-(2-cyano-6-(trifluoromethyl)phenyl)-1-neopentyl-1H-indol-3-yl)-2,2-difluoroethyl)cyclobutanesulfonamide (535)

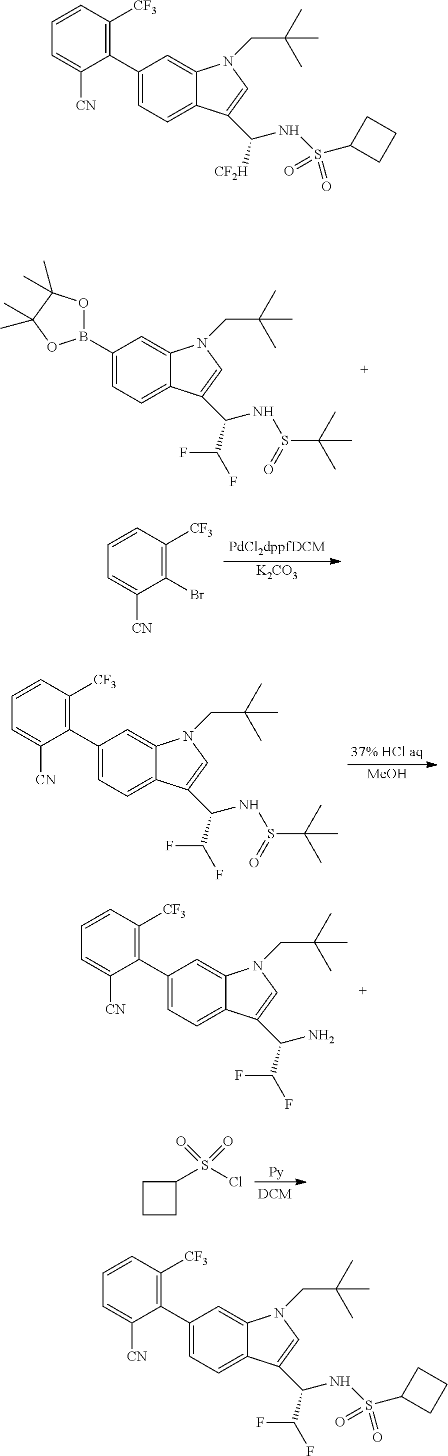

### Step 1: N—((S)-1-(6-(2-cyano-6-(trifluoromethyl)phenyl)-1-neopentyl-1H-indol-3-yl)-2,2-difluoroethyl)-2-methylpropane-2-sulfinamide

The title compound was prepared following the same general protocol as described for Step 5, Example 1, using N—((S)-2,2-difluoro-1-(1-neopentyl-6-(4,4,5,5-tetramethyl-1,3,2-dioxaborolan-2-yl)-1H-indol-3-yl)ethyl)-2-methylpropane-2-sulfinamide and 2-bromo-3-(trifluoromethyl)benzonitrile. ESI-MS (m/z): 540.2 [M+1]⁺.

### Step 2: (S)-2-(3-(1-amino-2,2-difluoroethyl)-1-neopentyl-1H-indol-6-yl)-3-(trifluoromethyl)benzonitrile

The title compound was prepared following the same general protocol as described for Step 4, Example 116, using N—((S)-1-(6-(2-cyano-6-(trifluoromethyl)phenyl)-1-neopentyl-1H-indol-3-yl)-2,2-difluoroethyl)-2-methylpropane-2-sulfinamide. ESI-MS (m/z): 419.2 [M-NH₂]⁺.

### Step 3: (S)—N-(1-(6-(2-cyano-6-(trifluoromethyl)phenyl)-1-neopentyl-1H-indol-3-yl)-2,2-difluoroethyl)cyclobutanesulfonamide

The title compound was prepared following the same general protocol as described for Step 5, Example 116, using (S)-2-(3-(1-amino-2,2-difluoroethyl)-1-neopentyl-1H-indol-6-yl)-3-(trifluoromethyl)benzonitrile and cyclobutanesulfonyl chloride. ESI-MS (m/z): 554.2 [M+1]⁺.

### Example 536: (S)—N-(2,2,2-trifluoro-1-(7-fluoro-1-neopentyl-6-(2-(trifluoromethyl)phenyl)-1H-indol-3-yl)ethyl)cyclopropanesulfonamide (536)

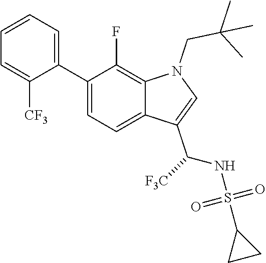 [SMILES: CC(C)(C)Cn1cc([C@H](NS(=O)(=O)C2CC2)C(F)(F)F)c2ccc(-c3ccccc3C(F)(F)F)c(F)c21]

### Step 1: 6-bromo-7-fluoro-1-neopentyl-1H-indole

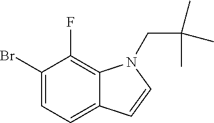 [SMILES: CC(C)(C)Cn1ccc2ccc(Br)c(F)c21]

The title compound was prepared following the same general protocol as described for Example 34, using 6-bromo-7-fluoro-1H-indole and 1-iodo-2,2-dimethylpropane. ESI-MS (m/z): 283.64, 285.45 [M+1]⁺.

### Step 2: (7-fluoro-1-neopentyl-1H-indol-6-yl)boronic acid

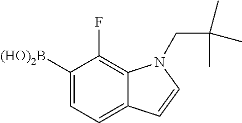 [SMILES: CC(C)(C)Cn1ccc2ccc(B(O)O)c(F)c21]

To a mixture of 6-bromo-7-fluoro-1-neopentyl-1H-indole (1.308 g, 4.605 mmol) and B(Oi-Pr)₃ (1.4 mL, 5.987 mmol) in THF (10 mL) at −78° C. under argon was added dropwise BuLi (4.0 mL, 2.5 M in hexane, 10.13 mmol). The reaction was monitored by anal. HPLC for completion. The mixture was quenched by the addition of saturated NH₄Cl and diluted with EtOAc. The aqueous layer was extracted with EtOAc (2×), and the combined organics were washed with brine, dried (Na₂SO₄) and concentrated. The crude was purified by silica gel to obtain the title compound. ESI-MS (m/z): 249.71 [M+1]⁺.

### Step 3: 7-fluoro-1-neopentyl-6-(2-(trifluoromethyl)phenyl)-1H-indole

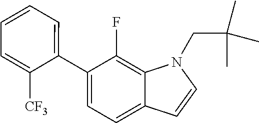 [SMILES: CC(C)(C)Cn1ccc2ccc(-c3ccccc3C(F)(F)F)c(F)c21]

The title compound was prepared following the same general protocol as described for Step 5, Example 1, using 1-bromo-2-(trifluoromethyl)benzene and (7-fluoro-1-neopentyl-1H-indol-6-yl)boronic acid. ESI-MS (m/z): 349.78 [M+1]⁺.

### Step 4: (S)-2-methyl-N—((S)-2,2,2-trifluoro-1-(7-fluoro-1-neopentyl-6-(2-(trifluoromethyl)phenyl)-1H-indol-3-yl)ethyl)propane-2-sulfinamide

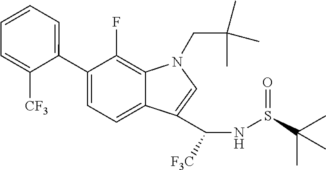 [SMILES: CC(C)(C)Cn1cc([C@H](N[S@@](=O)C(C)(C)C)C(F)(F)F)c2ccc(-c3ccccc3C(F)(F)F)c(F)c21]

The title compound was prepared following the same general protocol as described for Step 3, Example 116, using (S)-2-methyl-N-(3,3,3-trifluoroprop-1-en-1-yl)propane-2-sulfinamide and 7-fluoro-1-neopentyl-6-(2-(trifluoromethyl)phenyl)-1H-indole. ESI-MS (m/z): 550.58 [M+1]⁺.

### Step 5: (S)-2,2,2-trifluoro-1-(7-fluoro-1-neopentyl-6-(2-(trifluoromethyl)phenyl)-1H-indol-3-yl)ethan-1-amine

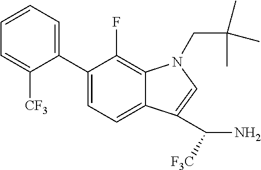 [SMILES: CC(C)(C)Cn1cc([C@H](N)C(F)(F)F)c2ccc(-c3ccccc3C(F)(F)F)c(F)c21]

The title compound was prepared following the same general protocol as described for Step 4, Example 116, using (S)-2-methyl-N—((S)-2,2,2-trifluoro-1-(7-fluoro-1-neopentyl-6-(2-(trifluoromethyl)phenyl)-1H-indol-3-yl)ethyl)propane-2-sulfinamide.

### Step 6: S)—N-(2,2,2-trifluoro-1-(7-fluoro-1-neopentyl-6-(2-(trifluoromethyl)phenyl)-1H-indol-3-yl)ethyl)cyclopropanesulfonamide

The title compound was prepared following the same general protocol as described for Step 5, Example 116, using (S)-2,2,2-trifluoro-1-(7-fluoro-1-neopentyl-6-(2-(trifluoromethyl)phenyl)-1H-indol-3-yl)ethan-1-amine. ¹HNMR (CDCl₃, 400 MHz) δ 7.78 (d, J=8.0 Hz, 1H), 7.59 (t, J=8.0 Hz, 1H), 7.54-7.48 (m, 2H), 7.36 (d, J=8.0 Hz, 1H), 7.18 (s, 1H), 6.99 (t, J=8.0 Hz, 1H), 5.44 (q, J=8.0 Hz, 1H), 5.05-4.95 (m, 1H), 4.19-3.99 (m, 2H), 2.60-2.40 (m, 1H), 1.30-1.09 (m, 2H), 0.98-0.83 (m, 11H)

### Example 537: 2,2-difluoro-N—((S)-2,2,2-trifluoro-1-(1-neopentyl-6-(2-(trifluoromethyl)phenyl)-1H-indol-3-yl)ethyl)cyclobutane-1-sulfonamide (537)

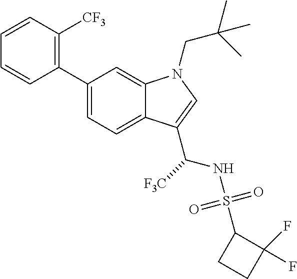 [SMILES: CC(C)(C)Cn1cc([C@H](NS(=O)(=O)C2CCC2(F)F)C(F)(F)F)c2ccc(-c3ccccc3C(F)(F)F)cc21]

The title compound was prepared following the same general protocol as described for Step 5, Example 116, using (S)-2,2,2-trifluoro-1-(1-neopentyl-6-(2-(trifluoromethyl)phenyl)-1H-indol-3-yl)ethan-1-amine and 2,2-difluorocyclobutane-1-sulfonyl chloride. HNMR (CDCl₃, 400 MHz) δ 7.77 (d, J=8.0 Hz, 1H), 7.65 (d, J=8.0 Hz, 1H), 7.56 (t, J=8.0 Hz, 1H), 7.46 (t, J=8.0 Hz, 1H), 7.39-7.23 (m, 3H), 7.11 (t, J=8.0 Hz, 1H), 5.15-4.84 (m, 1H), 4.33-4.18 (m, 1H), 4.00-3.84 (m, 2H), 3.75-3.35 (m, 1H), 3.24-1.61 (m, 4H), 0.99 (s, 9H)

### Example 538: (S)—N-(1-(6-(3-cyanopyridin-2-yl)-5-fluoro-1-neopentyl-1H-indol-3-yl)-2,2,2-trifluoroethyl)cyclobutanesulfonamide (538)

### Step 1: (S)-2-(3-(1-amino-2,2,2-trifluoroethyl)-5-fluoro-1-neopentyl-1H-indol-6-yl)nicotinonitrile

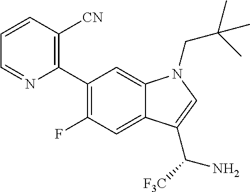 [SMILES: CC(C)(C)Cn1cc([C@H](N)C(F)(F)F)c2cc(F)c(-c3ncccc3C#N)cc21]

A mixture of rac-(1S)-1-[1-(2,2-dimethylpropyl)-5-fluoro-6-(4,4,5,5-tetramethyl-1,3,2-dioxaborolan-2-yl)indol-3-yl]-2,2,2-trifluoro-ethanamine (360 mg, 588.41 μmol) and 2-bromopyridine-3-carbonitrile (139.99 mg, 764.94 μmol) in dioxane (10 mL) and H2O (2 mL) was added Pd(dppf)Cl2 (43.07 mg, 58.84 μmol) and K2CO3 (162.40 mg, 1.18 mmol). The mixture was purged by N2 for 10 seconds. The mixture was stirred at 85° C. for 5 hours. The product was combined with another lot. The product was purified by chromatograph silica on gel (EA:PE, 0%˜60%, UV=254 nm). 2-[1-(2,2-dimethylpropyl)-5-fluoro-3-[rac-(1S)-1-amino-2,2,2-trifluoro-ethyl]indol-6-yl]pyridine-3-carbonitrile (230 mg, 569 μmol, 97% yield) as a yellow gum was obtained.

### Step 2: (S)—N-(1-(6-(3-cyanopyridin-2-yl)-5-fluoro-1-neopentyl-1H-indol-3-yl)-2,2,2-trifluoroethyl)cyclobutanesulfonamide

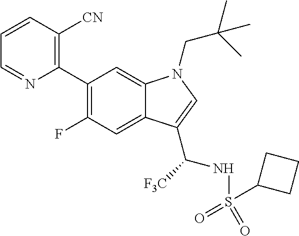 [SMILES: CC(C)(C)Cn1cc([C@H](NS(=O)(=O)C2CCC2)C(F)(F)F)c2cc(F)c(-c3ncccc3C#N)cc21]

A mixture of 2-[1-(2,2-dimethylpropyl)-5-fluoro-3-[rac-(1S)-1-amino-2,2,2-trifluoro-ethyl]indol-6-yl]pyridine-3-carbonitrile (230 mg, 568.74 μmol) in pyridine (2 mL) was added cyclobutanesulfonyl chloride (230 mg, 1.49 mmol). The mixture was purged by N2 for 20 seconds. The mixture was stirred at 100° C. for 4 hours under microwave and N2. The mixture was concentrated in vacuo. The product was purified by Prep HPLC. N-[rac-(1S)-1-[6-(3-cyano-2-pyridyl)-1-(2,2-dimethylpropyl)-5-fluoro-indol-3-yl]-2,2,2-trifluoro-ethyl]cyclobutanesulfonamide (7 mg, 13.40 μmol, 2% yield) as a light yellow solid was obtained. 1H NMR (400 MHz, CDCl3) δ 8.89 (dd, J=4.9, 1.7 Hz, 1H), 8.09 (dd, J=7.9, 1.8 Hz, 1H), 7.55 (s, 1H), 7.53 (d, J=5.1 Hz, 1H), 7.43 (dd, J=7.9, 4.9 Hz, 1H), 7.27 (s, 1H), 5.47 (d, J=7.6 Hz, 1H), 5.32 (p, J=7.7 Hz, 1H), 3.82 (dt, J=15.0, 10.9 Hz, 3H), 2.48 (ddd, J=18.1, 14.3, 9.3 Hz, 2H), 2.29-2.16 (m, 2H), 2.00-1.90 (m, 2H), 0.97 (s, 9H). MS Found: 523.2 [M+H]⁺.

### Example 541: (S)—N-(2,2-difluoro-1-(1-neopentyl-6-(2-(trifluoromethyl)phenyl)-1H-indol-3-yl)ethyl)cyclopropanesulfonamide (541)

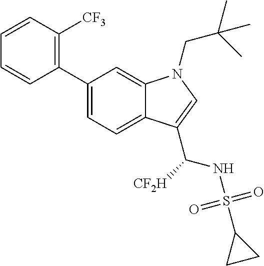 [SMILES: CC(C)(C)Cn1cc([C@H](NS(=O)(=O)C2CC2)C(F)F)c2ccc(-c3ccccc3C(F)(F)F)cc21]

The title compound was prepared following the same general protocol as described for Step 5, Example 116, using (S)-2,2-difluoro-1-(1-neopentyl-6-(2-(trifluoromethyl)phenyl)-1H-indol-3-yl)ethan-1-amine and cyclopropanesulfonyl chloride. ESI-MS (m/z): 515.2 [M+1]⁺.

### Example 542: (S)—N-(2,2-difluoro-1-(5-fluoro-1-neopentyl-6-(2-(trifluoromethyl)phenyl)-1H-indol-3-yl)ethyl)cyclopropanesulfonamide (542)

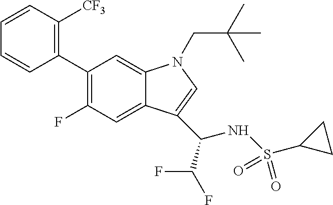 [SMILES: CC(C)(C)Cn1cc([C@H](NS(=O)(=O)C2CC2)C(F)F)c2cc(F)c(-c3ccccc3C(F)(F)F)cc21]

The title compound was prepared following the same general protocol as described for Step 5, Example 116, using (S)-2,2-difluoro-1-(5-fluoro-1-neopentyl-6-(2-(trifluoromethyl)phenyl)-1H-indol-3-yl)ethan-1-amine and cyclopropanesulfonyl chloride. ESI-MS (m/z): 533.2 [M+1]⁺.

### Example 543: (S)—N-(2,2-difluoro-1-(6-(2-fluoro-6-(trifluoromethyl)phenyl)-1-neopentyl-1H-indol-3-yl)ethyl)cyclopropanesulfonamide (543)

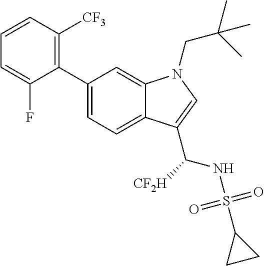 [SMILES: CC(C)(C)Cn1cc([C@H](NS(=O)(=O)C2CC2)C(F)F)c2ccc(-c3c(F)cccc3C(F)(F)F)cc21]

The title compound was prepared following the same general protocol as described for Step 5, Example 116, using (S)-2,2-difluoro-1-(6-(2-fluoro-6-(trifluoromethyl)phenyl)-1-neopentyl-1H-indol-3-yl)ethan-1-amine and cyclopropanesulfonyl chloride. ESI-MS (m/z): 533.2 [M+1]⁺.

### Example 544: (S)—N-(2,2,2-trifluoro-1-(7-fluoro-1-neopentyl-6-(2-(trifluoromethyl)phenyl)-1H-indol-3-yl)ethyl)cyclobutanesulfonamide (544)

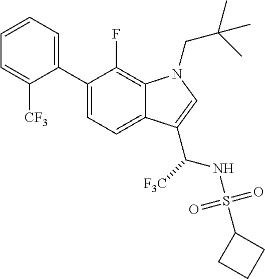 [SMILES: CC(C)(C)Cn1cc([C@H](NS(=O)(=O)C2CCC2)C(F)(F)F)c2ccc(-c3ccccc3C(F)(F)F)c(F)c21]

The title compound was prepared following the same general protocol as described for Step 6, Example 536 (Compound 536), using ((S)-2,2,2-trifluoro-1-(7-fluoro-1-neopentyl-6-(2-(trifluoromethyl)phenyl)-1H-indol-3-yl)ethan-1-amine and cyclobutanesulfonyl chloride. ¹HNMR (CDCl₃, 400 MHz) δ 7.78 (t, J=8.0 Hz, 1H), 7.53-7.47 (d, J=8.0 Hz, 1H), 7.56 (m, 2H), 7.38-7.35 (m, 1H), 7.17 (s, 1H), 6.99 (t, J=8.0 Hz, 1H), 5.35 (q, J=8.0 Hz, 1H), 4.90-4.82 (m, 1H), 4.20-4.15 (m, 1H), 4.04-3.98 (m, 1H), 3.85-3.74 (m, 1H), 2.53-2.41 (m, 2H), 2.24-2.13 (m, 2H), 2.01-1.86 (m, 2H), 0.99 (s, 9H)

### Example 545: (S)—N-(1-(6-(2-cyano-6-(trifluoromethyl)phenyl)-1-neopentyl-1H-indol-3-yl)-2,2-difluoroethyl)cyclopropanesulfonamide (545)

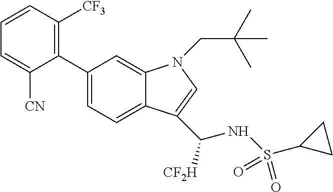 [SMILES: CC(C)(C)Cn1cc([C@H](NS(=O)(=O)C2CC2)C(F)F)c2ccc(-c3c(C#N)cccc3C(F)(F)F)cc21]

The title compound was prepared following the same general protocol as described for Step 5, Example 116, using (S)-2-(3-(1-amino-2,2-difluoroethyl)-1-neopentyl-1H-indol-6-yl)-3-(trifluoromethyl)benzonitrile and cyclopropanesulfonyl chloride. ESI-MS (m/z): 540.2 [M+1]⁺.

### Example 546: (S)—N-(2,2-difluoro-1-(5-fluoro-1-neopentyl-6-(2-(trifluoromethyl)phenyl)-1H-pyrrolo[2,3-b]pyridin-3-yl)ethyl)cyclopropanesulfonamide (546)

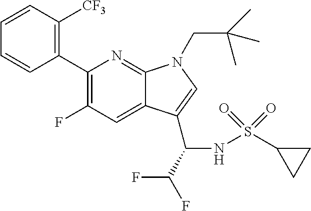 [SMILES: CC(C)(C)Cn1cc([C@H](NS(=O)(=O)C2CC2)C(F)F)c2cc(F)c(-c3ccccc3C(F)(F)F)nc21]

The title compound was prepared following the same general protocol as described for Step 5, Example 116, using (S)-2,2-difluoro-1-(5-fluoro-1-neopentyl-6-(2-(trifluoromethyl)phenyl)-1H-pyrrolo[2,3-b]pyridin-3-yl)ethan-1-amine and cyclopropanesulfonyl chloride. ESI-MS (m/z): 534.2 [M+1]⁺.

### Example 547: (S)—N-(2,2-difluoro-1-(1-neopentyl-6-(2-(trifluoromethyl)phenyl)-1H-pyrrolo[2,3-b]pyridin-3-yl)ethyl)cyclopropanesulfonamide (547)

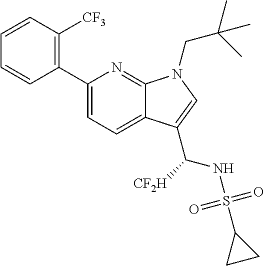 [SMILES: CC(C)(C)Cn1cc([C@H](NS(=O)(=O)C2CC2)C(F)F)c2ccc(-c3ccccc3C(F)(F)F)nc21]

The title compound was prepared following the same general protocol as described for Step 5, Example 116, using (S)-2,2-difluoro-1-(1-neopentyl-6-(2-(trifluoromethyl)phenyl)-1H-pyrrolo[2,3-b]pyridin-3-yl)ethan-1-amine and cyclopropanesulfonyl chloride. ESI-MS (m/z): 516.2 [M+1]⁺.

### Example 550: (S)—N-(2,2-difluoro-1-(1-neopentyl-6-(2-(trifluoromethyl)phenyl)-1H-indol-3-yl)ethyl-1-d)cyclopropanesulfonamide (550)

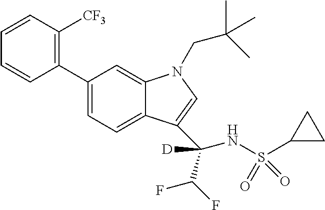 [SMILES: [2H][C@](NS(=O)(=O)C1CC1)(c1cn(CC(C)(C)C)c2cc(-c3ccccc3C(F)(F)F)ccc12)C(F)F]

The title compound was prepared following the same general protocol as described for Step 5, Example 116, using (S)-2,2-difluoro-1-(1-neopentyl-6-(2-(trifluoromethyl)phenyl)-1H-indol-3-yl)ethan-1-d-1-amine and cyclopropanesulfonyl chloride. ESI-MS (m/z): 516.2 [M+1]⁺.

### Example 551: (S)—N-(2,2-difluoro-1-(6-(4-fluoro-2-(trifluoromethyl)phenyl)-1-neopentyl-1H-indol-3-yl)ethyl)cyclopropanesulfonamide (551)

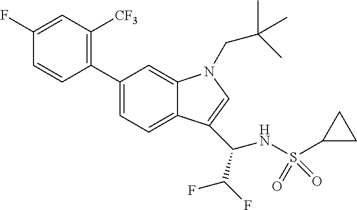 [SMILES: CC(C)(C)Cn1cc([C@H](NS(=O)(=O)C2CC2)C(F)F)c2ccc(-c3ccc(F)cc3C(F)(F)F)cc21]

The title compound was prepared following the same general protocol as described for Step 5, Example 116, using (S)-2,2-difluoro-1-(6-(4-fluoro-2-(trifluoromethyl)phenyl)-1-neopentyl-1H-indol-3-yl)ethan-1-amine and cyclopropanesulfonyl chloride. ESI-MS (m/z): 533.2 [M+1]⁺.

### Example 552: (S)—N-(2,2-difluoro-1-(7-fluoro-1-neopentyl-6-(2-(trifluoromethyl)phenyl)-1H-indol-3-yl)ethyl)cyclobutanesulfonamide (552)

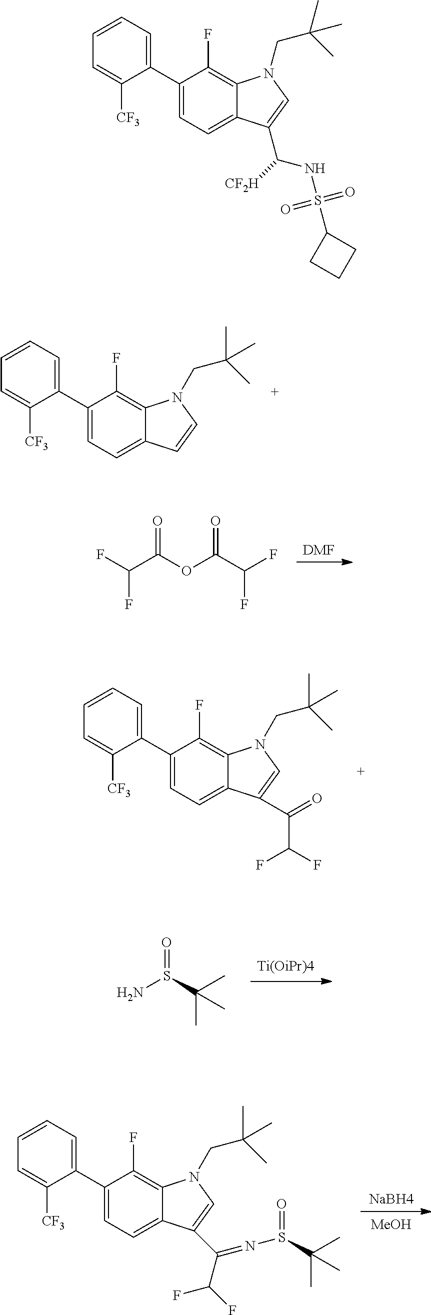

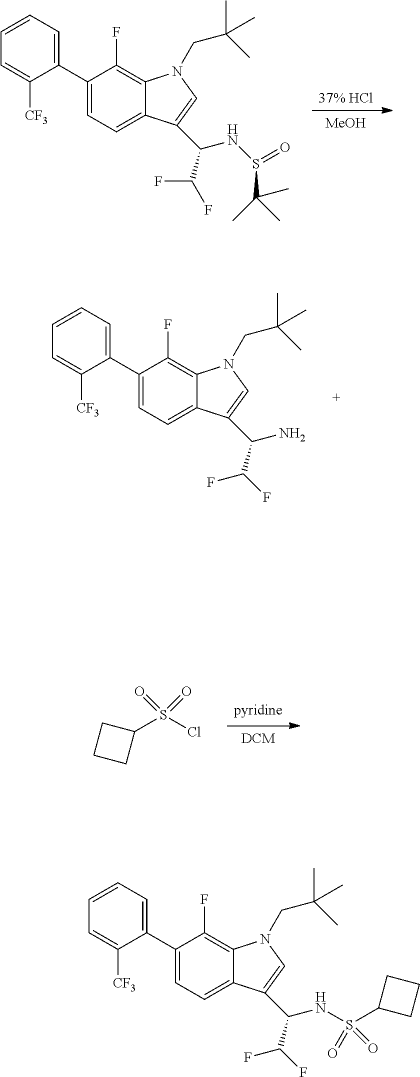

### Step 1: 2,2-difluoro-1-(7-fluoro-1-neopentyl-6-(2-(trifluoromethyl)phenyl)-1H-indol-3-yl)ethan-1-one

The title compound was prepared following the same general protocol as described for Step 1, Example 128, using 7-fluoro-1-neopentyl-6-(2-(trifluoromethyl)phenyl)-1H-indole and 2,2-difluoroacetic anhydride. ESI-MS (m/z): 428.1 [M+1]⁺.

### Step 2: (S)—N-(2,2-difluoro-1-(7-fluoro-1-neopentyl-6-(2-(trifluoromethyl)phenyl)-1H-indol-3-yl)ethylidene)-2-methylpropane-2-sulfinamide

The title compound was prepared following the same general protocol as described for Step 4, Example 113, using 2,2-difluoro-1-(7-fluoro-1-neopentyl-6-(2-(trifluoromethyl)phenyl)-1H-indol-3-yl)ethan-1-one. ESI-MS (m/z): 531.2 [M+1]⁺.

### Step 3: (S)—N—((S)-2,2-difluoro-1-(7-fluoro-1-neopentyl-6-(2-(trifluoromethyl)phenyl)-1H-indol-3-yl)ethyl)-2-methylpropane-2-sulfinamide

The title compound was prepared following the same general protocol as described for Step 3, Example 420, using (S,E)-N-(2,2-difluoro-1-(7-fluoro-1-neopentyl-6-(2-(trifluoromethyl)phenyl)-1H-indol-3-yl)ethylidene)-2-methylpropane-2-sulfinamide. ESI-MS (m/z): 533.2 [M+1]⁺.

### Step 4: (S)-2,2-difluoro-1-(7-fluoro-1-neopentyl-6-(2-(trifluoromethyl)phenyl)-1H-indol-3-yl)ethan-1-amine

The title compound was prepared following the same general protocol as described for Step 4, Example 116, using (S)—N—((S)-2,2-difluoro-1-(7-fluoro-1-neopentyl-6-(2-(trifluoromethyl)-phenyl)-1H-indol-3-yl)ethyl)-2-methylpropane-2-sulfinamide. ESI-MS (m/z): 412.2 [M-NH₂]⁺.

### Step 5: (S)—N-(2,2-difluoro-1-(7-fluoro-1-neopentyl-6-(2-(trifluoromethyl)phenyl)-1H-indol-3-yl)ethyl)cyclobutanesulfonamide

The title compound was prepared following the same general protocol as described for Step 5, Example 116, using (S)-2,2-difluoro-1-(7-fluoro-1-neopentyl-6-(2-(trifluoromethyl)phenyl)-1H-indol-3-yl)ethan-1-amine and cyclobutanesulfonyl chloride. ESI-MS (m/z): 547.2 [M+1]f.

### Example 553: (S)—N-(2,2-difluoro-1-(7-fluoro-1-neopentyl-6-(2-(trifluoromethyl)phenyl)-1H-indol-3-yl)ethyl)cyclopropanesulfonamide (553)

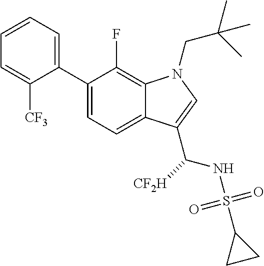 [SMILES: CC(C)(C)Cn1cc([C@H](NS(=O)(=O)C2CC2)C(F)F)c2ccc(-c3ccccc3C(F)(F)F)c(F)c21]

The title compound was prepared following the same general protocol as described for Step 5, Example 116, using (S)-2,2-difluoro-1-(7-fluoro-1-neopentyl-6-(2-(trifluoromethyl)phenyl)-1H-indol-3-yl)ethan-1-amine and cyclopropanesulfonyl chloride. ESI-MS (m/z): 533.2 [M+1]f.

### Example 555: (S)—N-(2,2-difluoro-1-(5-fluoro-1-neopentyl-6-(2-(trifluoromethyl)pyridin-3-yl)-1H-indol-3-yl)ethyl)cyclopropanesulfonamide (555)

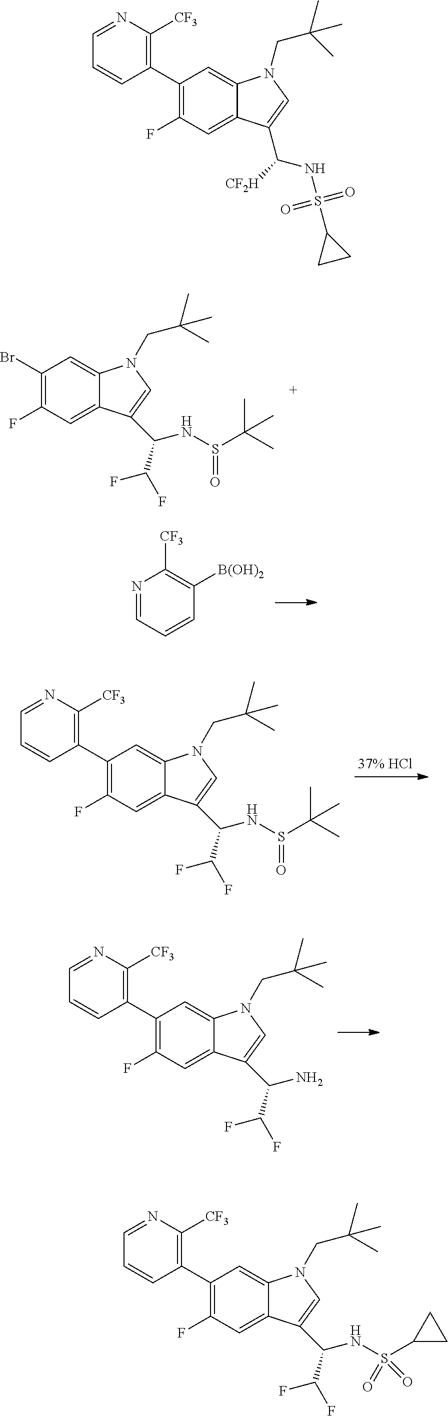

### Step 1: N—((S)-2,2-difluoro-1-(5-fluoro-1-neopentyl-6-(2-(trifluoromethyl)pyridin-3-yl)-1H-indol-3-yl)ethyl)-2-methylpropane-2-sulfinamide

The title compound was prepared following the same general protocol as described for Step 5, Example 1, using N—((S)-1-(6-bromo-5-fluoro-1-neopentyl-1H-indol-3-yl)-2,2-difluoroethyl)-2-methylpropane-2-sulfinamide and (2-(trifluoromethyl)pyridin-3-yl)boronic acid. ESI-MS (m/z): 534.2 [M+1]⁺.

### Step 2: (S)-2,2-difluoro-1-(5-fluoro-1-neopentyl-6-(2-(trifluoromethyl)pyridin-3-yl)-1H-indol-3-yl)ethan-1-amine

The title compound was prepared following the same general protocol as described for Step 4, Example 116, using N—((S)-2,2-difluoro-1-(5-fluoro-1-neopentyl-6-(2-(trifluoromethyl)pyridin-3-yl)-1H-indol-3-yl)ethyl)-2-methylpropane-2-sulfinamide. ESI-MS (m/z): 413.2 [M-NH₂]⁺.

### Step 3: (S)—N-(2,2-difluoro-1-(5-fluoro-1-neopentyl-6-(2-(trifluoromethyl)pyridin-3-yl)-1H-indol-3-yl)ethyl)cyclopropanesulfonamide

The title compound was prepared following the same general protocol as described for Step 5, Example 116, using (S)-2,2-difluoro-1-(5-fluoro-1-neopentyl-6-(2-(trifluoromethyl)pyridin-3-yl)-1H-indol-3-yl)ethan-1-amine and cyclopropanesulfonyl chloride. ESI-MS (m/z): 534.2 [M+1]⁺.

### Example 557: (S)—N-(2,2-difluoro-1-(5-fluoro-1-neopentyl-6-(3-(trifluoromethyl)pyridin-2-yl)-1H-indol-3-yl)ethyl)cyclopropanesulfonamide (557)

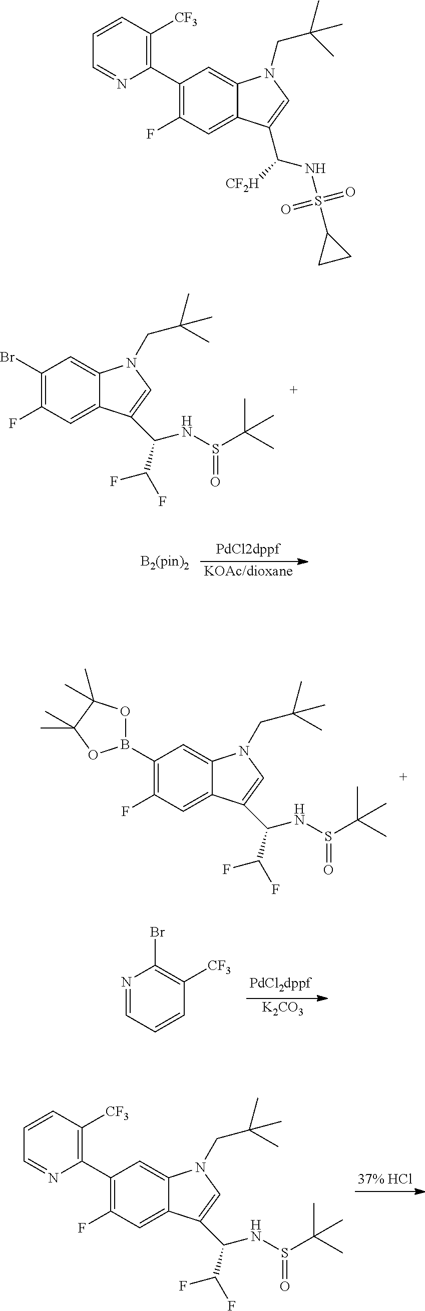

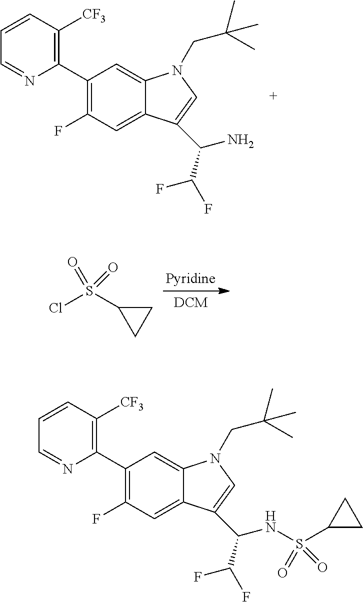 [SMILES: CC(C)(C)Cn1cc([C@H](N)C(F)F)c2cc(F)c(-c3ncccc3C(F)(F)F)cc21 ; O=S(=O)(Cl)C1CC1 ; CC(C)(C)Cn1cc([C@H](NS(=O)(=O)C2CC2)C(F)F)c2cc(F)c(-c3ncccc3C(F)(F)F)cc21]

### Step 1: N—((S)-2,2-difluoro-1-(5-fluoro-1-neopentyl-6-(4,4,5,5-tetramethyl-1,3,2-dioxaborolan-2-yl)-1H-indol-3-yl)ethyl)-2-methylpropane-2-sulfinamide

The title compound was prepared following the same general protocol as described for Step 2, Example 33, using N—((S)-1-(6-bromo-5-fluoro-1-neopentyl-1H-indol-3-yl)-2,2-difluoroethyl)-2-methylpropane-2-sulfinamide. ESI-MS (m/z): 515.3 [M+1]⁺.

### Step 2: N—((S)-2,2-difluoro-1-(5-fluoro-1-neopentyl-6-(3-(trifluoromethyl)pyridin-2-yl)-1H-indol-3-yl)ethyl)-2-methylpropane-2-sulfinamide

The title compound was prepared following the same general protocol as described for Step 5, Example 1, using N—((S)-2,2-difluoro-1-(5-fluoro-1-neopentyl-6-(4,4,5,5-tetramethyl-1,3,2-dioxaborolan-2-yl)-1H-indol-3-yl)ethyl)-2-methylpropane-2-sulfinamide and 2-bromo-3-(trifluoromethyl)pyridine. ESI-MS (m/z): 534.2 [M+1]⁺.

### Step 3: (S)-2,2-difluoro-1-(5-fluoro-1-neopentyl-6-(3-(trifluoromethyl)pyridin-2-yl)-1H-indol-3-yl)ethan-1-amine

The title compound was prepared following the same general protocol as described for Step 4, Example 116, using N—((S)-2,2-difluoro-1-(5-fluoro-1-neopentyl-6-(3-(trifluoromethyl)pyridin-2-yl)-1H-indol-3-yl)ethyl)-2-methylpropane-2-sulfinamide. ESI-MS (m/z): 413.2 [M-NH₂]⁺.

### Step 4: (S)—N-(2,2-difluoro-1-(5-fluoro-1-neopentyl-6-(3-(trifluoromethyl)pyridin-2-yl)-1H-indol-3-yl)ethyl)cyclopropanesulfonamide

The title compound was prepared following the same general protocol as described for Step 5, Example 116, using (S)-2,2-difluoro-1-(5-fluoro-1-neopentyl-6-(3-(trifluoromethyl)pyridin-2-yl)-1H-indol-3-yl)ethan-1-amine and cyclopropanesulfonyl chloride. ESI-MS (m/z): 534.2 [M+1]⁺.

### Example 558: (S)—N-(1-(6-(3-cyanopyridin-2-yl)-5-fluoro-1-neopentyl-1H-indol-3-yl)-2,2-difluoroethyl)cyclopropanesulfonamide (558)

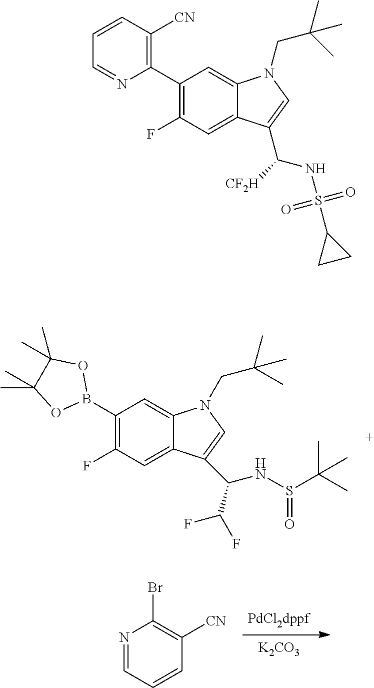

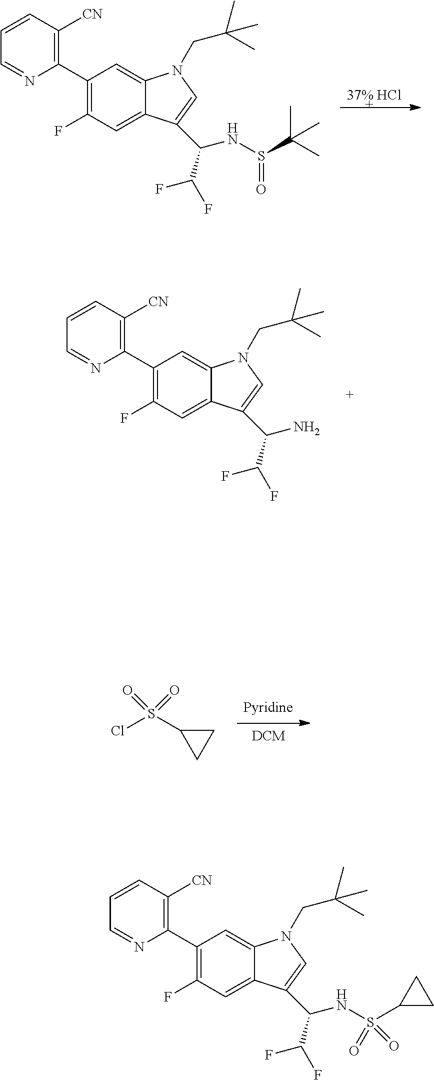

### Step 1: (R)—N—((S)-1-(6-(3-cyanopyridin-2-yl)-5-fluoro-1-neopentyl-1H-indol-3-yl)-2,2-difluoroethyl)-2-methylpropane-2-sulfinamide

The title compound was prepared following the same general protocol as described for Step 5, Example 1, using N—((S)-2,2-difluoro-1-(5-fluoro-1-neopentyl-6-(4,4,5,5-tetramethyl-1,3,2-dioxaborolan-2-yl)-1H-indol-3-yl)ethyl)-2-methylpropane-2-sulfinamide and 2-bromonicotino-nitrile. ESI-MS (m/z): 491.2 [M+1]⁺.

### Step 2: (S)-2-(3-(1-amino-2,2-difluoroethyl)-5-fluoro-1-neopentyl-1H-indol-6-yl)nicotinonitrile

The title compound was prepared following the same general protocol as described for Step 4, Example 116, using (R)—N—((S)-1-(6-(3-cyanopyridin-2-yl)-5-fluoro-1-neopentyl-1H-indol-3-yl)-2,2-difluoroethyl)-2-methylpropane-2-sulfinamide. ESI-MS (m/z): 370.2 [M-NH₂]⁺.

### Step 3: (S)—N-(1-(6-(3-cyanopyridin-2-yl)-5-fluoro-1-neopentyl-1H-indol-3-yl)-2,2-difluoroethyl)cyclopropanesulfonamide

The title compound was prepared following the same general protocol as described for Step 5, Example 116, using (S)-2-(3-(1-amino-2,2-difluoroethyl)-5-fluoro-1-neopentyl-1H-indol-6-yl)nicotinonitrile and cyclopropanesulfonyl chloride. ESI-MS (m/z): 491.2 [M+1]⁺.

### Example 562: (S)—N-(2,2-difluoro-1-(6-(4-fluoro-2-(trifluoromethyl)phenyl)-1-neopentyl-1H-indol-3-yl)ethyl)cyclobutanesulfonamide (562)

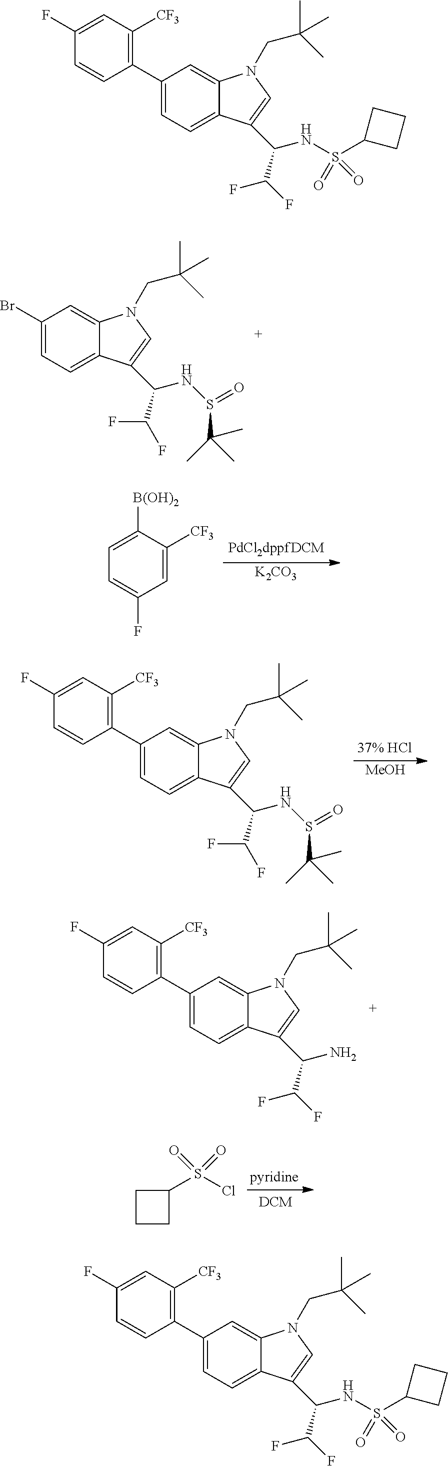

### Step 1: (S)—N—((S)-2,2-difluoro-1-(6-(4-fluoro-2-(trifluoromethyl)phenyl)-1-neopentyl-1H-indol-3-yl)ethyl)-2-methylpropane-2-sulfinamide

The title compound was prepared following the same general protocol as described for Step 5, Example 1, using (S)—N—((S)-1-(6-bromo-1-neopentyl-1H-indol-3-yl)-2,2-difluoroethyl)-2-methylpropane-2-sulfinamide and (4-fluoro-2-(trifluoromethyl)phenyl)boronic acid. ESI-MS (m/z): 533.2 [M+1]⁺.

### Step 2: (S)-2,2-difluoro-1-(6-(4-fluoro-2-(trifluoromethyl)phenyl)-1-neopentyl-1H-indol-3-yl)ethan-1-amine

The title compound was prepared following the same general protocol as described for Step 4, Example 116, using (S)—N—((S)-2,2-difluoro-1-(6-(4-fluoro-2-(trifluoromethyl)phenyl)-1-neopentyl-1H-indol-3-yl)ethyl)-2-methylpropane-2-sulfinamide. ESI-MS (m/z): 412.2 [M-NH2]⁺.

### Step 3: (S)—N-(2,2-difluoro-1-(6-(4-fluoro-2-(trifluoromethyl)phenyl)-1-neopentyl-1H-indol-3-yl)ethyl)cyclobutanesulfonamide

The title compound was prepared following the same general protocol as described for Step 5, Example 116, using (S)-2,2-difluoro-1-(6-(4-fluoro-2-(trifluoromethyl)phenyl)-1-neopentyl-1H-indol-3-yl)ethan-1-amine and cyclobutanesulfonyl chloride. ESI-MS (m/z): 547.2 [M+1]⁺.

### Example 563: (S)—N-(2,2-difluoro-1-(1-neopentyl-6-(2-(trifluoromethyl)phenyl)-1H-indol-3-yl)ethyl-1-d)cyclobutanesulfonamide (563)

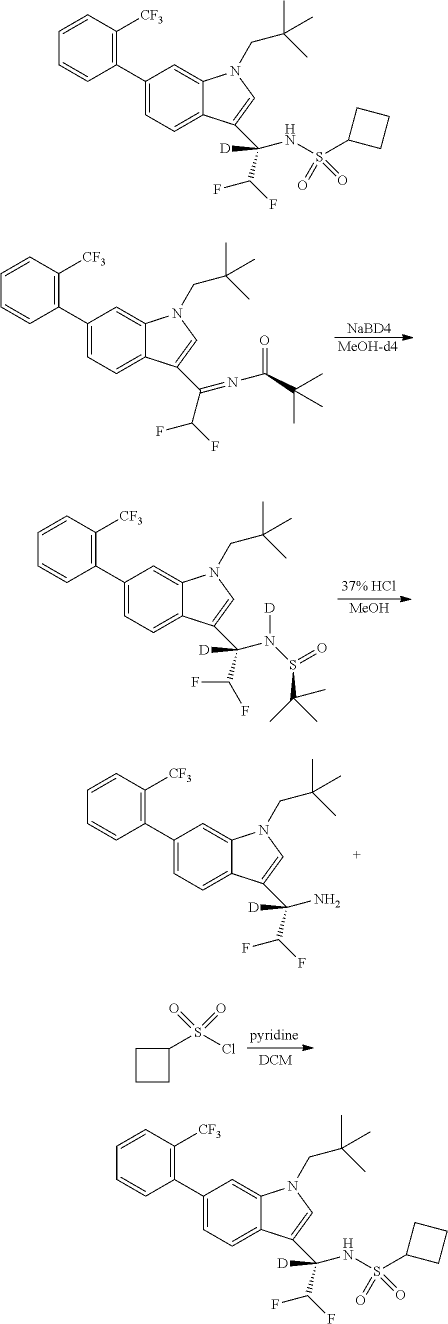

### Step 1: (S)—N—((S)-2,2-difluoro-1-(1-neopentyl-6-(2-(trifluoromethyl)phenyl)-1H-indol-3-yl)ethyl-1-d)-2-methylpropane-2-sulfinamide-d

The title compound was prepared following the same general protocol as described for Step 3, Example 420 (Compound 420), using N-(2,2-difluoro-1-(1-neopentyl-6-(2-(trifluoromethyl)phenyl)-1H-indol-3-yl)ethylidene)pivalamide and NaBH₄ in MeOH-d4. ESI-MS (m/z): 517.2 [M+1]+.

### Step 2: (S)-2,2-difluoro-1-(1-neopentyl-6-(2-(trifluoromethyl)phenyl)-1H-indol-3-yl)ethan-1-d-1-amine

The title compound was prepared following the same general protocol as described for Step 4, Example 116, using (S)—N—((S)-2,2-difluoro-1-(1-neopentyl-6-(2-(trifluoromethyl)phenyl)-1H-indol-3-yl)ethyl-1-d)-2-methylpropane-2-sulfinamide-d. ESI-MS (m/z): 395.2 [M-NH₂]⁺.

### Step 3: (S)—N-(2,2-difluoro-1-(1-neopentyl-6-(2-(trifluoromethyl)phenyl)-1H-indol-3-yl)ethyl-1-d)cyclobutanesulfonamide

The title compound was prepared following the same general protocol as described for Step 5, Example 116, using (S)-2,2-difluoro-1-(1-neopentyl-6-(2-(trifluoromethyl)phenyl)-1H-indol-3-yl)ethan-1-d-1-amine and cyclobutanesulfonyl chloride. ESI-MS (m/z): 530.2 [M+1]⁺.

### Example 564: (S)—N-(1-(1-(2,2-dimethylpropyl-1,1-d2)-6-(2-(trifluoromethyl)phenyl)-1H-indol-3-yl)-2,2,2-trifluoroethyl)cyclopropanesulfonamide (564)

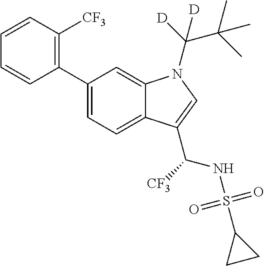 [SMILES: [2H]C([2H])(n1cc([C@H](NS(=O)(=O)C2CC2)C(F)(F)F)c2ccc(-c3ccccc3C(F)(F)F)cc21)C(C)(C)C]

The title compound was prepared following the same general protocol as described for Step 5, Example 116, using (S)-1-(1-(2,2-dimethylpropyl-1,1-d2)-6-(2-(trifluoromethyl)phenyl)-1H-indol-3-yl)-2,2,2-trifluoroethan-1-amine and cyclopropanesulfonyl chloride. ESI-MS (m/z): 535.2 [M+1]⁺.

### Example 565: (S)—N-(1-(6-(2-cyanophenyl)-1-neopentyl-1H-pyrrolo[2,3-b]pyridin-3-yl)-2,2,2-trifluoroethyl)cyclopropanesulfonamide (565)

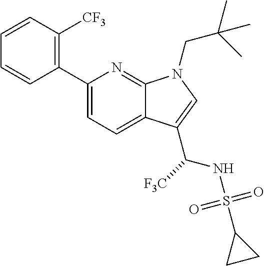 [SMILES: CC(C)(C)Cn1cc([C@H](NS(=O)(=O)C2CC2)C(F)(F)F)c2ccc(-c3ccccc3C(F)(F)F)nc21]

The title compound was prepared following the same general protocol as described for Step 5, Example 491, using (S)-2-(3-(1-amino-2,2,2-trifluoroethyl)-1-neopentyl-1H-pyrrolo[2,3-b]pyridin-6-yl)benzonitrile and cyclopropanesulfonyl chloride. ESI-MS (m/z): 491.2 [M+1]⁺.

### Example 566: (S)—N-(2,2-difluoro-1-(5-fluoro-6-(4-fluoro-2-(trifluoromethyl)phenyl)-1-neopentyl-1H-indol-3-yl)ethyl)cyclopropanesulfonamide (566)

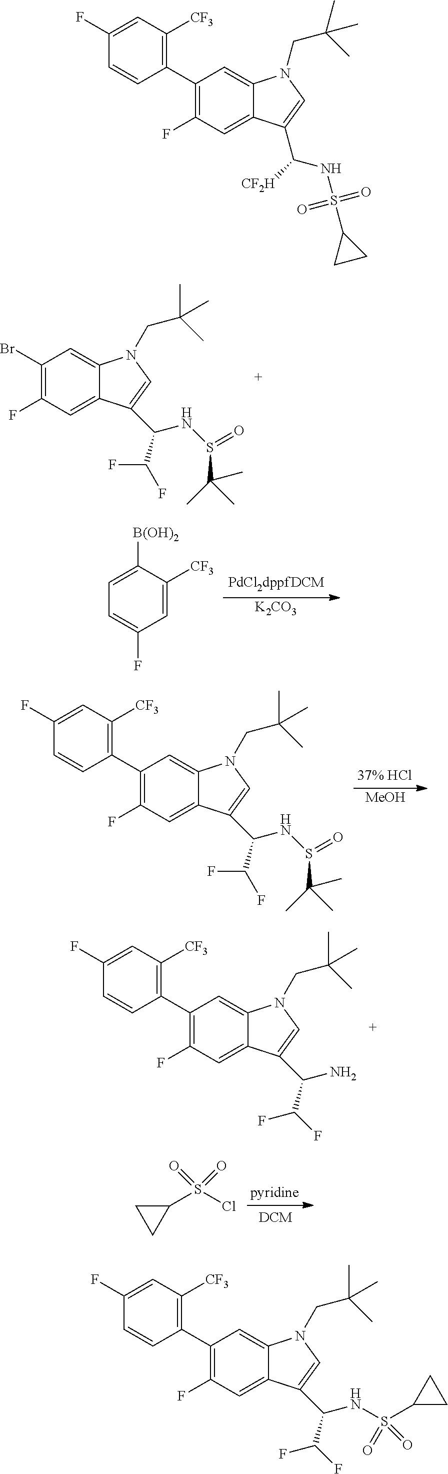

### Step 1: (S)—N—((S)-2,2-difluoro-1-(5-fluoro-6-(4-fluoro-2-(trifluoromethyl)phenyl)-1-neopentyl-1H-indol-3-yl)ethyl)-2-methylpropane-2-sulfinamide

The title compound was prepared following the same general protocol as described for Step 5, Example 1, using (S)—N—((S)-1-(6-bromo-5-fluoro-1-neopentyl-1H-indol-3-yl)-2,2-difluoroethyl)-2-methylpropane-2-sulfinamide and (4-fluoro-2-(trifluoromethyl)phenyl)boronic acid. ESI-MS (m/z): 551.2 [M+1]⁺.

### Step 2: (S)-2,2-difluoro-1-(5-fluoro-6-(4-fluoro-2-(trifluoromethyl)phenyl)-1-neopentyl-1H-indol-3-yl)ethan-1-amine

The title compound was prepared following the same general protocol as described for Step 4, Example 116, using (S)—N—((S)-2,2-difluoro-1-(5-fluoro-6-(4-fluoro-2-(trifluoromethyl)phenyl)-1-neopentyl-1H-indol-3-yl)ethyl)-2-methylpropane-2-sulfinamide. ESI-MS (m/z): 430.2 [M-NH2]⁺.

### Step 3: (S)—N-(2,2-difluoro-1-(5-fluoro-6-(4-fluoro-2-(trifluoromethyl)phenyl)-1-neopentyl-1H-indol-3-yl)ethyl)cyclopropanesulfonamide

The title compound was prepared following the same general protocol as described for Step 5, Example 116, using (S)-2,2-difluoro-1-(5-fluoro-6-(4-fluoro-2-(trifluoromethyl)phenyl)-1-neopentyl-1H-indol-3-yl)ethan-1-amine and cyclopropanesulfonyl chloride. ESI-MS (m/z): 573.1 [M+Na]⁺.

### Example 567: (R)—N-((6-(2-cyano-5-fluorophenyl)-1-neopentyl-1H-indol-3-yl)((difluoro-l3-methyl)-l2-fluoraneyl)methyl)cyclopropanesulfonamide (567)

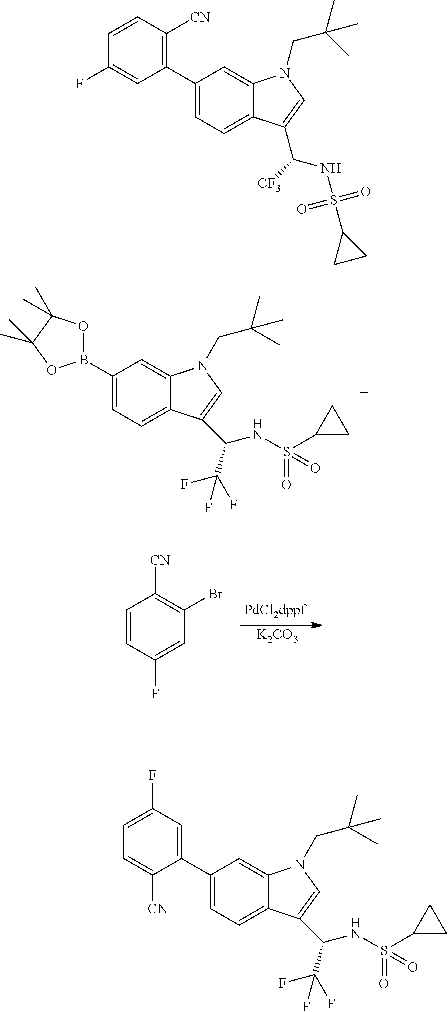

The title compound was prepared following the same general protocol as described for Step 5, Example 1, using (S)—N-(2,2,2-trifluoro-1-(1-neopentyl-6-(4,4,5,5-tetramethyl-1,3,2-dioxaborolan-2-yl)-1H-indol-3-yl)ethyl)cyclopropanesulfonamide and 2-bromo-4-fluorobenzo-nitrile. ESI-MS (m/z): 508.2 [M+1]⁺.

### Example 568: (S)—N-(2,2-difluoro-1-(6-(4-fluoro-2-(trifluoromethyl)phenyl)-1-neopentyl-1H-pyrrolo[2,3-b]pyridin-3-yl)ethyl)cyclopropanesulfonamide (568)

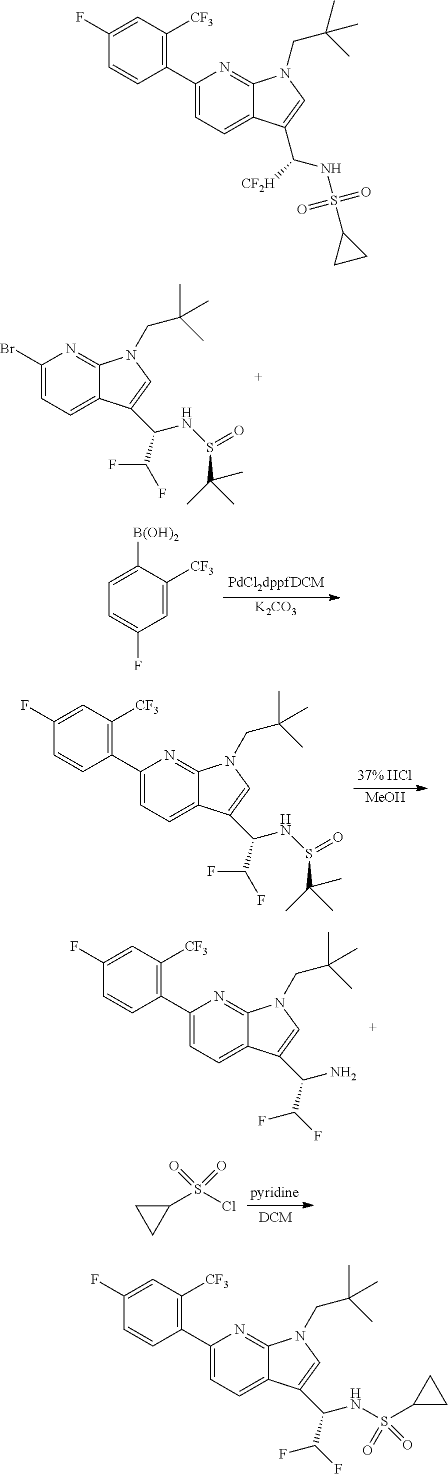

### Step 1: (S)—N—((S)-2,2-difluoro-1-(6-(4-fluoro-2-(trifluoromethyl)phenyl)-1-neopentyl-1H-pyrrolo[2,3-b]pyridin-3-yl)ethyl)-2-methylpropane-2-sulfinamide

The title compound was prepared following the same general protocol as described for Step 5, Example 1, using (S)—N—((S)-1-(6-bromo-1-neopentyl-1H-pyrrolo[2,3-b]pyridin-3-yl)-2,2-difluoroethyl)-2-methylpropane-2-sulfinamide and (4-fluoro-2-(trifluoromethyl)phenyl)boronic acid. ESI-MS (m/z): 534.2 [M+1]⁺.

### Step 2: (S)-2,2-difluoro-1-(6-(4-fluoro-2-(trifluoromethyl)phenyl)-1-neopentyl-1H-pyrrolo[2,3-b]pyridin-3-yl)ethan-1-amine

The title compound was prepared following the same general protocol as described for Step 4, Example 116, using (S)—N—((S)-2,2-difluoro-1-(6-(4-fluoro-2-(trifluoromethyl)phenyl)-1-neopentyl-1H-pyrrolo[2,3-b]pyridin-3-yl)ethyl)-2-methylpropane-2-sulfinamide. ESI-MS (m/z): 413.2 [M-NH₂]⁺.

### Step 3: (S)—N-(2,2-difluoro-1-(6-(4-fluoro-2-(trifluoromethyl)phenyl)-1-neopentyl-1H-pyrrolo[2,3-b]pyridin-3-yl)ethyl)cyclopropanesulfonamide

The title compound was prepared following the same general protocol as described for Step 5, Example 116, using (S)-2,2-difluoro-1-(6-(4-fluoro-2-(trifluoromethyl)phenyl)-1-neopentyl-1H-pyrrolo[2,3-b]pyridin-3-yl)ethan-1-amine and cyclopropanesulfonyl chloride. ESI-MS (m/z): 534.2 [M+1]⁺.

### Biological Examples

Cell culture. HEK293 were purchased from American Type Culture Collection (ATCC) and cultured in in DMEM supplemented with 10% FBS, 2 mM L-glutamine, and 1% penicillin/streptomycin at 37° C., 5% CO₂ under standard culture conditions.

Luciferase reporter assays. HEK293 cells were plated 24 hours prior to transfection in 10 cm dishes at a density of 350,000 cells/10 ml/plate. Transfections were performed using Lipofectamine 3000 (Invitrogen) according to manufacturer's protocol. Cells were co-transfected with pG5-UAS, pCMV-Gal4-REV-ERBα (residues 206-614, containing the hinge and the ligand binding domain), along with pACT (Promega) fused with the mouse NCOR receptor interaction domain (RID, residues 1828-2471) or pACT empty vector expressing the VP-16 fusion protein only as a control. Sixteen hours post-transfection, cells were trypsinized, counted and plated at 10,000 cells/20 ul/well in 384 well plates. 4 h post cell plating, cells were treated with vehicle or compound. Ligand stocks were prepared via serial dilution in DMSO, added to 8 wells per concentration, and plates were incubated at 37° C. Twenty-four hours post-treatment, luciferase activity was measured using BriteLite (PerkinElmer Life and Analytical Sciences) and read using an Envision multilabel plate reader (PerkinElmer Life and Analytical Sciences). All values were normalized to DMSO to produce fold induction values. Data were plotted using GraphPad Prism.

TR-FRET corepressor interaction assay. The time-resolved fluorescence resonance energy transfer (TR-FRET) assay was performed in black low-volume 384-well plates (Greiner). Each well contained 4 nM 6×Histag-REV-ERBα LBD (human; residues 281-614 Δ324-422) or 6×Histag-REV-ERBα LBD (human; residues 381-579) protein expressed in and purified from Escherichia coli using nickel affinity and size exclusion chromatography; 1 nM LanthaScreen Elite Tb-anti-HIS Antibody (Thermo Fisher Scientific); and 400 nM FITC-labeled peptide derived from the SMRT corepressor (TNMGLEAIIRKALMGKYDQWEE) containing a N-terminal FITC label with a six-carbon linker (Ahx) and an amidated C-terminus for stability (synthesized by LifeTein) in TR-FRET buffer (20 mM potassium phosphate, pH 7.4, 50 mM potassium chloride, 1 mM dithiothreitol, and 0.005% Tween-20). Ligand stocks were prepared via serial dilution in DMSO, added to wells in triplicate, and plates were incubated at 4° C. for 2 h and read using a BioTek Synergy Neo multimode plate reader. The Tb donor was excited at 340 nm; its fluorescence emission was monitored at 495 nm, and the acceptor FITC emission was measured at 520 nm; and the TR-FRET ratio was calculated as the signal at 520 nm divided by the signal at 495 nm. Data were plotted using GraphPad Prism as TR-FRET ratio vs. ligand concentration and fit to sigmoidal dose response curve equation.

Exemplary compounds of the present disclosure were assayed as described above to assess agonism of REV-ERBα and REV-ERBβ. Results of the FRET assay are shown in Table 2 below (A: EC₅₀<500 nM; B: EC₅₀ 500 nM-3 μM; C: EC₅₀>3 μM; D: not tested).

TABLE 2
Agonism of REV-ERBα and REV-ERBβ.

| Compound | Structure | REVERBα | REVERBβ |
|---|---|---|---|
| 1 | 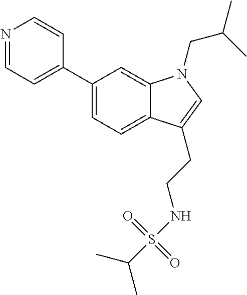 [SMILES: CC(C)Cn1cc(CCNS(=O)(=O)C(C)C)c2ccc(-c3ccncc3)cc21] | A | D |
| 2 | 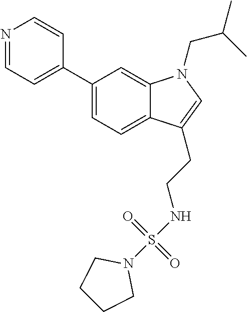 [SMILES: CC(C)Cn1cc(CCNS(=O)(=O)N2CCCC2)c2ccc(-c3ccncc3)cc21] | A | D |
| 3 | 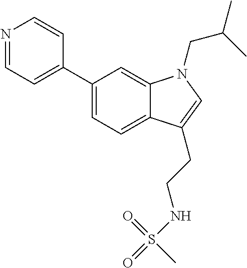 [SMILES: CC(C)Cn1cc(CCNS(C)(=O)=O)c2ccc(-c3ccncc3)cc21] | B | D |
| 5 | 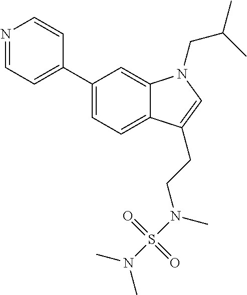 [SMILES: CC(C)Cn1cc(CCN(C)S(=O)(=O)N(C)C)c2ccc(-c3ccncc3)cc21] | A | D |
| 6 | 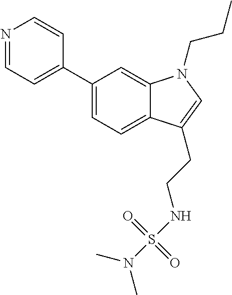 [SMILES: CCCn1cc(CCNS(=O)(=O)N(C)C)c2ccc(-c3ccncc3)cc21] | A | D |
| 7 | 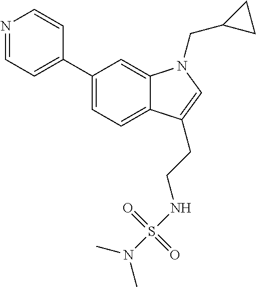 [SMILES: CN(C)S(=O)(=O)NCCc1cn(CC2CC2)c2cc(-c3ccncc3)ccc12] | A | D |
| 8 | 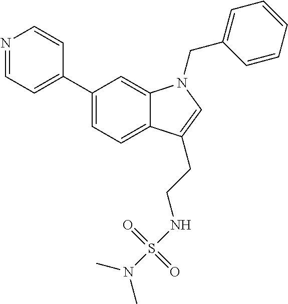 [SMILES: CN(C)S(=O)(=O)NCCc1cn(Cc2ccccc2)c2cc(-c3ccncc3)ccc12] | A | D |
| 9 | 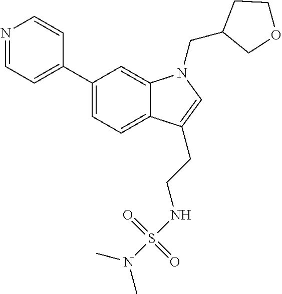 [SMILES: CN(C)S(=O)(=O)NCCc1cn(CC2CCOC2)c2cc(-c3ccncc3)ccc12] | B | D |
| 11 | 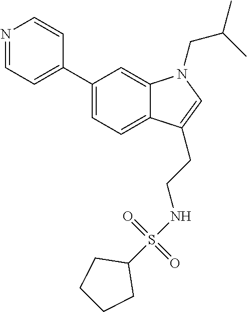 [SMILES: CC(C)Cn1cc(CCNS(=O)(=O)C2CCCC2)c2ccc(-c3ccncc3)cc21] | A | C |
| 12 | 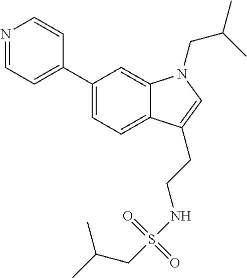 [SMILES: CC(C)Cn1cc(CCNS(=O)(=O)CC(C)C)c2ccc(-c3ccncc3)cc21] | A | C |
| 13 | 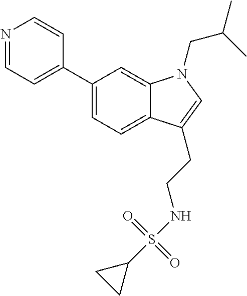 [SMILES: CC(C)Cn1cc(CCNS(=O)(=O)C2CC2)c2ccc(-c3ccncc3)cc21] | A | C |
| 14 | 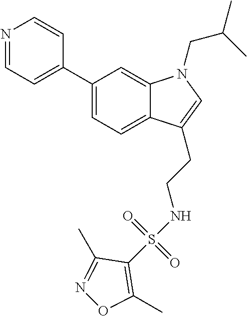 [SMILES: Cc1noc(C)c1S(=O)(=O)NCCc1cn(CC(C)C)c2cc(-c3ccncc3)ccc12] | C | C |
| 15 | 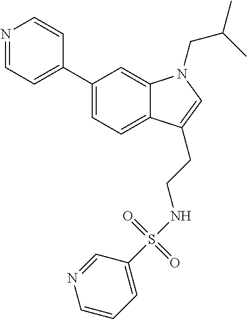 [SMILES: CC(C)Cn1cc(CCNS(=O)(=O)c2cccnc2)c2ccc(-c3ccncc3)cc21] | B | C |
| 16 | 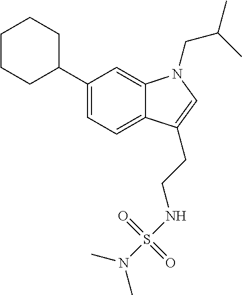 [SMILES: CC(C)Cn1cc(CCNS(=O)(=O)N(C)C)c2ccc(C3CCCCC3)cc21] | A | B |
| 17 | 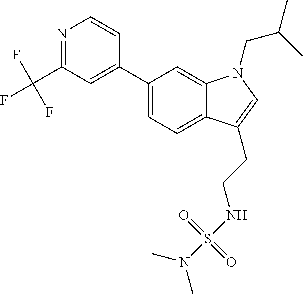 [SMILES: CC(C)Cn1cc(CCNS(=O)(=O)N(C)C)c2ccc(-c3ccnc(C(F)(F)F)c3)cc21] | B | C |
| 18 | 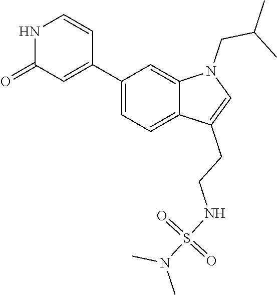 [SMILES: CC(C)Cn1cc(CCNS(=O)(=O)N(C)C)c2ccc(-c3cc[nH]c(=O)c3)cc21] | C | C |
| 19 | 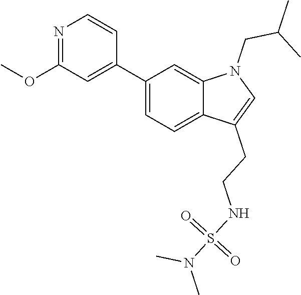 [SMILES: COc1cc(-c2ccc3c(CCNS(=O)(=O)N(C)C)cn(CC(C)C)c3c2)ccn1] | B | D |
| 20 | 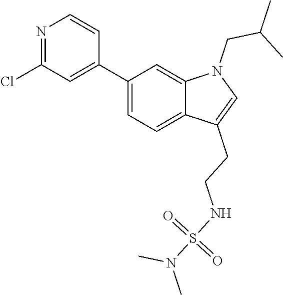 [SMILES: CC(C)Cn1cc(CCNS(=O)(=O)N(C)C)c2ccc(-c3ccnc(Cl)c3)cc21] | A | C |
| 21 | 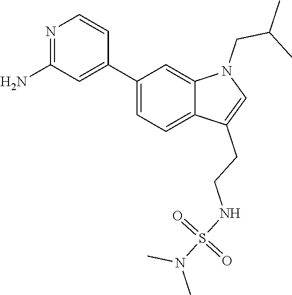 [SMILES: CC(C)Cn1cc(CCNS(=O)(=O)N(C)C)c2ccc(-c3ccnc(N)c3)cc21] | B | C |
| 22 | 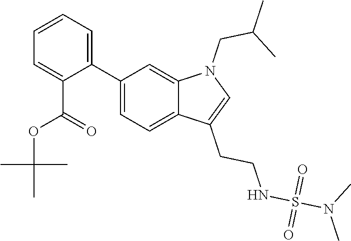 [SMILES: CC(C)Cn1cc(CCNS(=O)(=O)N(C)C)c2ccc(-c3ccccc3C(=O)OC(C)(C)C)cc21] | B | D |
| 23 | 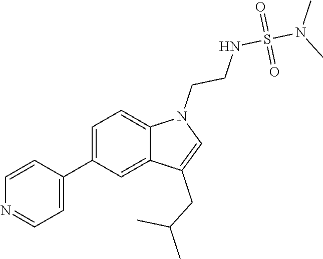 [SMILES: CC(C)Cc1cn(CCNS(=O)(=O)N(C)C)c2ccc(-c3ccncc3)cc12] | A | B |
| 24 | 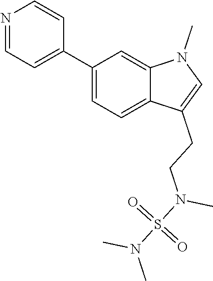 [SMILES: CN(C)S(=O)(=O)N(C)CCc1cn(C)c2cc(-c3ccncc3)ccc12] | B | D |
| 25 | 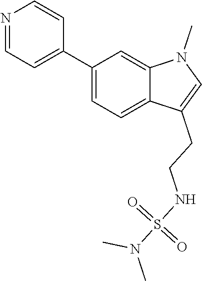 [SMILES: CN(C)S(=O)(=O)NCCc1cn(C)c2cc(-c3ccncc3)ccc12] | B | D |
| 26 | 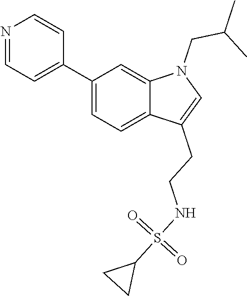 [SMILES: CC(C)Cn1cc(CCNS(=O)(=O)C2CC2)c2ccc(-c3ccncc3)cc21] | A | D |
| 27 | 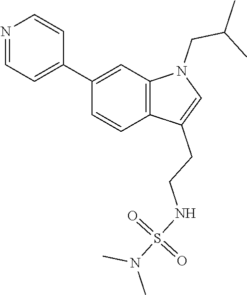 [SMILES: CC(C)Cn1cc(CCNS(=O)(=O)N(C)C)c2ccc(-c3ccncc3)cc21] | A | B |
| 28 | 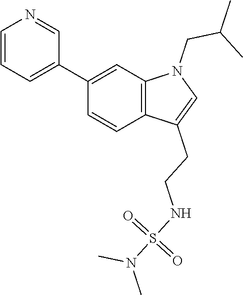 [SMILES: CC(C)Cn1cc(CCNS(=O)(=O)N(C)C)c2ccc(-c3cccnc3)cc21] | A | C |
| 29 | 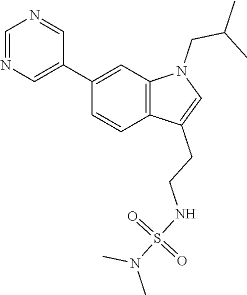 [SMILES: CC(C)Cn1cc(CCNS(=O)(=O)N(C)C)c2ccc(-c3cncnc3)cc21] | B | C |
| 30 | 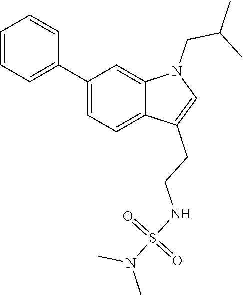 [SMILES: CC(C)Cn1cc(CCNS(=O)(=O)N(C)C)c2ccc(-c3ccccc3)cc21] | A | B |
| 31 | 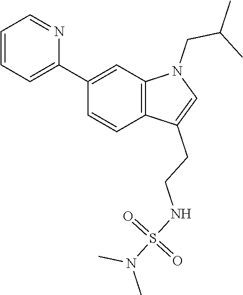 [SMILES: CC(C)Cn1cc(CCNS(=O)(=O)N(C)C)c2ccc(-c3ccccn3)cc21] | B | C |
| 32 | 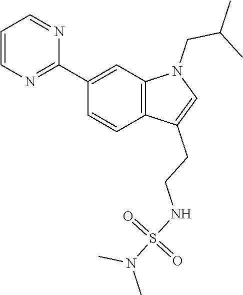 [SMILES: CC(C)Cn1cc(CCNS(=O)(=O)N(C)C)c2ccc(-c3ncccn3)cc21] | B | C |
| 33 | 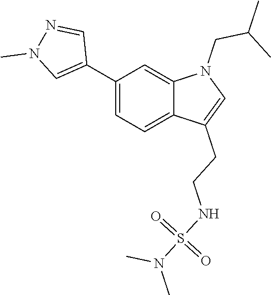 [SMILES: CC(C)Cn1cc(CCNS(=O)(=O)N(C)C)c2ccc(-c3cnn(C)c3)cc21] | C | C |
| 34 | 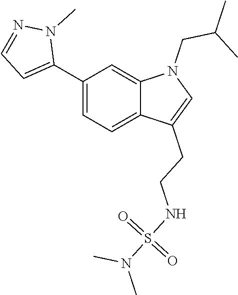 [SMILES: CC(C)Cn1cc(CCNS(=O)(=O)N(C)C)c2ccc(-c3ccnn3C)cc21] | A | C |
| 35 | 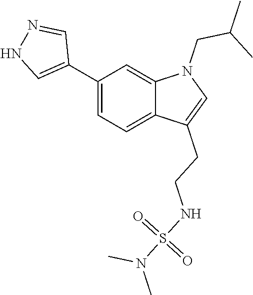 [SMILES: CC(C)Cn1cc(CCNS(=O)(=O)N(C)C)c2ccc(-c3cn[nH]c3)cc21] | A | C |
| 36 | 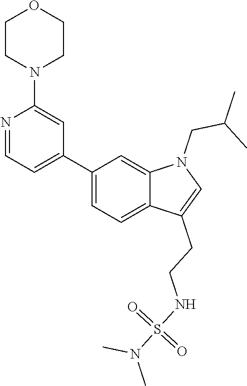 [SMILES: CC(C)Cn1cc(CCNS(=O)(=O)N(C)C)c2ccc(-c3ccnc(N4CCOCC4)c3)cc21] | C | C |
| 37 | 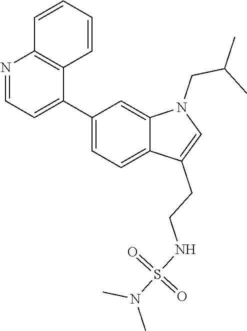 [SMILES: CC(C)Cn1cc(CCNS(=O)(=O)N(C)C)c2ccc(-c3ccnc4ccccc34)cc21] | A | B |
| 38 | 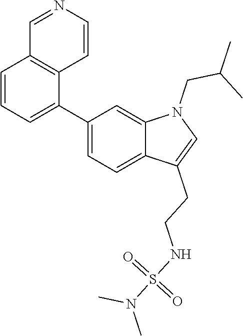 [SMILES: CC(C)Cn1cc(CCNS(=O)(=O)N(C)C)c2ccc(-c3cccc4cnccc34)cc21] | C | C |
| 39 | 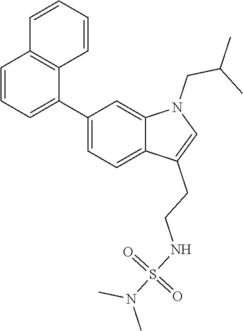 [SMILES: CC(C)Cn1cc(CCNS(=O)(=O)N(C)C)c2ccc(-c3cccc4ccccc34)cc21] | A | C |
| 40 | 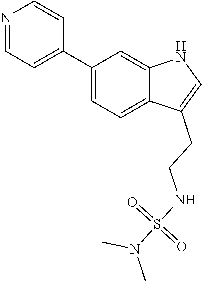 [SMILES: CN(C)S(=O)(=O)NCCc1c[nH]c2cc(-c3ccncc3)ccc12] | C | D |
| 41 | 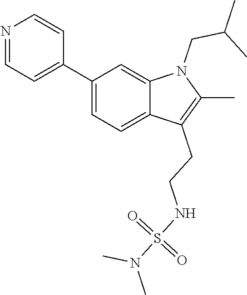 [SMILES: Cc1c(CCNS(=O)(=O)N(C)C)c2ccc(-c3ccncc3)cc2n1CC(C)C] | C | D |
| 42 | 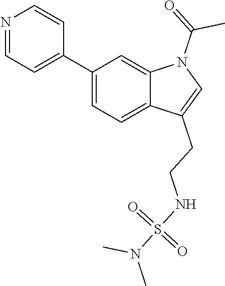 [SMILES: CC(=O)n1cc(CCNS(=O)(=O)N(C)C)c2ccc(-c3ccncc3)cc21] | C | D |
| 43 | 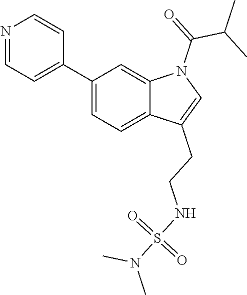 [SMILES: CC(C)C(=O)n1cc(CCNS(=O)(=O)N(C)C)c2ccc(-c3ccncc3)cc21] | A | D |
| 44 | 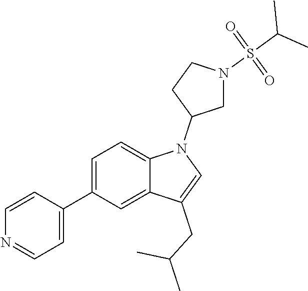 [SMILES: CC(C)Cc1cn(C2CCN(S(=O)(=O)C(C)C)C2)c2ccc(-c3ccncc3)cc12] | B | D |
| 45 | 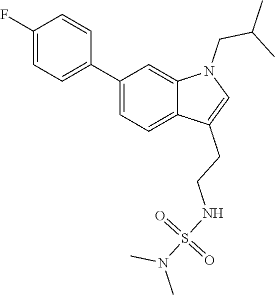 [SMILES: CC(C)Cn1cc(CCNS(=O)(=O)N(C)C)c2ccc(-c3ccc(F)cc3)cc21] | B | D |
| 46 | 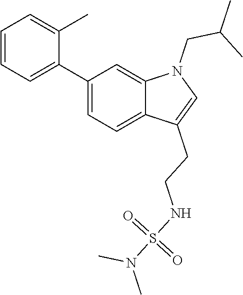 [SMILES: Cc1ccccc1-c1ccc2c(CCNS(=O)(=O)N(C)C)cn(CC(C)C)c2c1] | A | D |
| 47 | 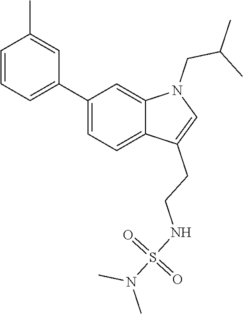 [SMILES: Cc1cccc(-c2ccc3c(CCNS(=O)(=O)N(C)C)cn(CC(C)C)c3c2)c1] | B | D |
| 48 | 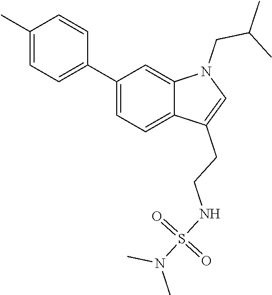 [SMILES: Cc1ccc(-c2ccc3c(CCNS(=O)(=O)N(C)C)cn(CC(C)C)c3c2)cc1] | B | D |
| 49 | 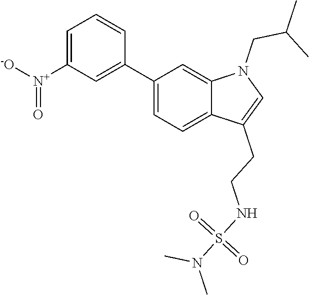 | A | D |
| 50 | 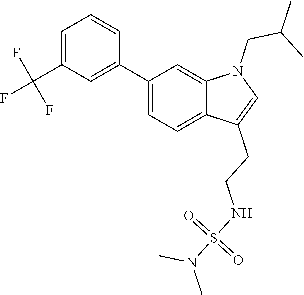 [SMILES: CC(C)Cn1cc(CCNS(=O)(=O)N(C)C)c2ccc(-c3cccc(C(F)(F)F)c3)cc21] | B | D |
| 51 | 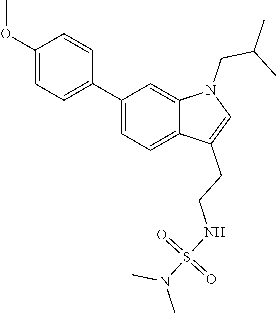 [SMILES: COc1ccc(-c2ccc3c(CCNS(=O)(=O)N(C)C)cn(CC(C)C)c3c2)cc1] | B | D |
| 52 | 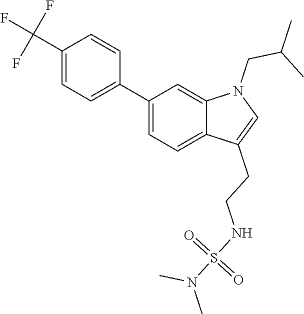 [SMILES: CC(C)Cn1cc(CCNS(=O)(=O)N(C)C)c2ccc(-c3ccc(C(F)(F)F)cc3)cc21] | C | D |
| 53 | 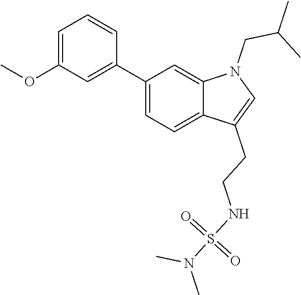 [SMILES: COc1cccc(-c2ccc3c(CCNS(=O)(=O)N(C)C)cn(CC(C)C)c3c2)c1] | B | D |
| 54 | 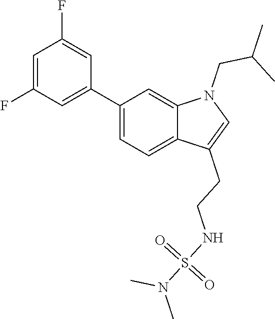 [SMILES: CC(C)Cn1cc(CCNS(=O)(=O)N(C)C)c2ccc(-c3cc(F)cc(F)c3)cc21] | B | D |
| 55 | 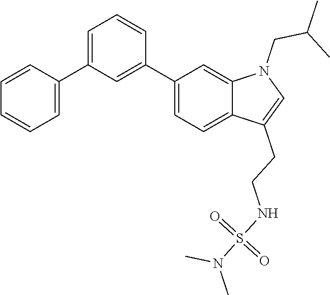 [SMILES: CC(C)Cn1cc(CCNS(=O)(=O)N(C)C)c2ccc(-c3cccc(-c4ccccc4)c3)cc21] | B | D |
| 56 | 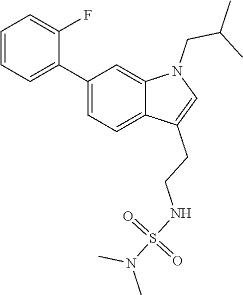 [SMILES: CC(C)Cn1cc(CCNS(=O)(=O)N(C)C)c2ccc(-c3ccccc3F)cc21] | B | D |
| 57 | 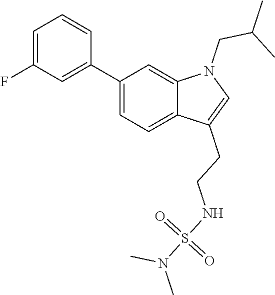 [SMILES: CC(C)Cn1cc(CCNS(=O)(=O)N(C)C)c2ccc(-c3cccc(F)c3)cc21] | B | D |
| 58 | 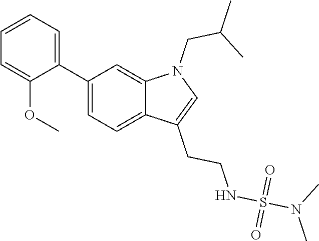 [SMILES: COc1ccccc1-c1ccc2c(CCNS(=O)(=O)N(C)C)cn(CC(C)C)c2c1] | A | C |
| 59 | 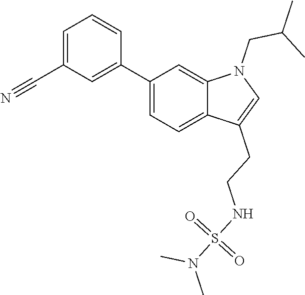 [SMILES: CC(C)Cn1cc(CCNS(=O)(=O)N(C)C)c2ccc(-c3cccc(C#N)c3)cc21] | A | D |
| 60 | 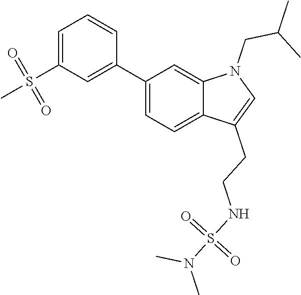 [SMILES: CC(C)Cn1cc(CCNS(=O)(=O)N(C)C)c2ccc(-c3cccc(S(C)(=O)=O)c3)cc21] | B | D |
| 61 | 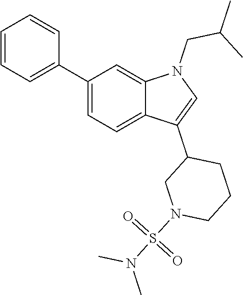 [SMILES: CC(C)Cn1cc(C2CCCN(S(=O)(=O)N(C)C)C2)c2ccc(-c3ccccc3)cc21] | B | D |
| 62 | 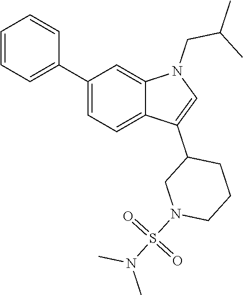 [SMILES: CC(C)Cn1cc(C2CCCN(S(=O)(=O)N(C)C)C2)c2ccc(-c3ccccc3)cc21] | B | D |
| 63 | 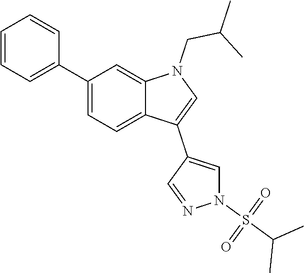 [SMILES: CC(C)Cn1cc(-c2cnn(S(=O)(=O)C(C)C)c2)c2ccc(-c3ccccc3)cc21] | C | D |
| 64 | 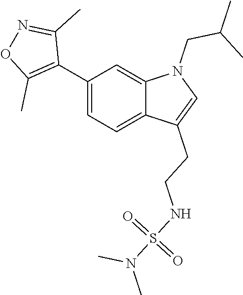 [SMILES: Cc1noc(C)c1-c1ccc2c(CCNS(=O)(=O)N(C)C)cn(CC(C)C)c2c1] | C | D |
| 65 | 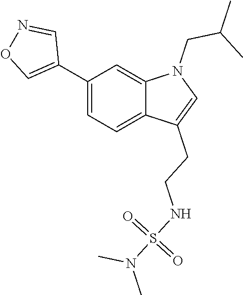 [SMILES: CC(C)Cn1cc(CCNS(=O)(=O)N(C)C)c2ccc(-c3cnoc3)cc21] | B | D |
| 66 | 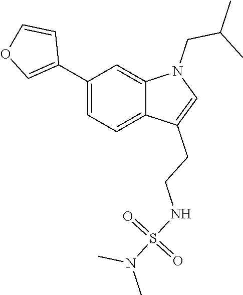 [SMILES: CC(C)Cn1cc(CCNS(=O)(=O)N(C)C)c2ccc(-c3ccoc3)cc21] | A | D |
| 67 | 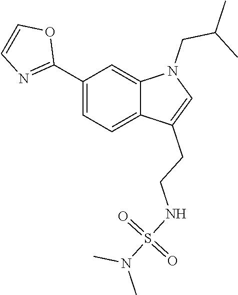 [SMILES: CC(C)Cn1cc(CCNS(=O)(=O)N(C)C)c2ccc(-c3ncco3)cc21] | B | D |
| 68 | 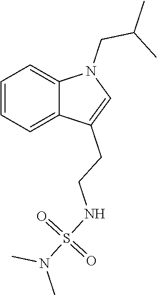 [SMILES: CC(C)Cn1cc(CCNS(=O)(=O)N(C)C)c2ccccc21] | B | C |
| 69 | 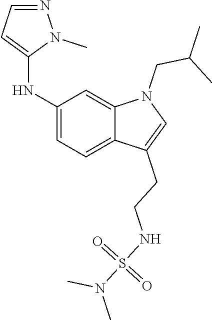 [SMILES: CC(C)Cn1cc(CCNS(=O)(=O)N(C)C)c2ccc(Nc3ccnn3C)cc21] | C | D |
| 70 | 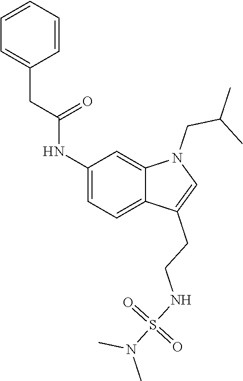 [SMILES: CC(C)Cn1cc(CCNS(=O)(=O)N(C)C)c2ccc(NC(=O)Cc3ccccc3)cc21] | C | D |
| 71 | 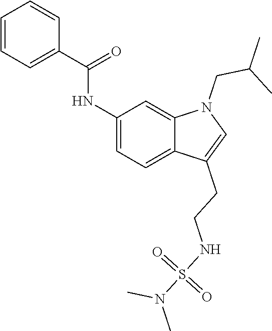 [SMILES: CC(C)Cn1cc(CCNS(=O)(=O)N(C)C)c2ccc(NC(=O)c3ccccc3)cc21] | C | D |
| 72 | 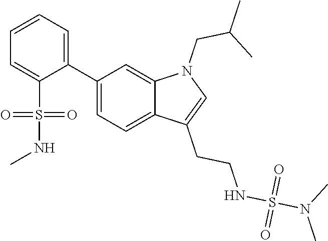 [SMILES: CNS(=O)(=O)c1ccccc1-c1ccc2c(CCNS(=O)(=O)N(C)C)cn(CC(C)C)c2c1] | B | D |
| 73 | 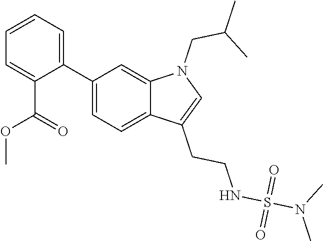 [SMILES: COC(=O)c1ccccc1-c1ccc2c(CCNS(=O)(=O)N(C)C)cn(CC(C)C)c2c1] | C | D |
| 74 | 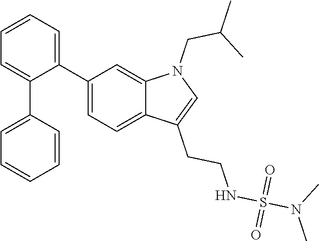 [SMILES: CC(C)Cn1cc(CCNS(=O)(=O)N(C)C)c2ccc(-c3ccccc3-c3ccccc3)cc21] | C | D |
| 75 | 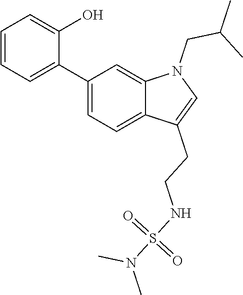 [SMILES: CC(C)Cn1cc(CCNS(=O)(=O)N(C)C)c2ccc(-c3ccccc3O)cc21] | B | D |
| 76 | 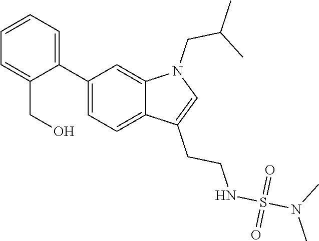 [SMILES: CC(C)Cn1cc(CCNS(=O)(=O)N(C)C)c2ccc(-c3ccccc3CO)cc21] | B | D |
| 77 | 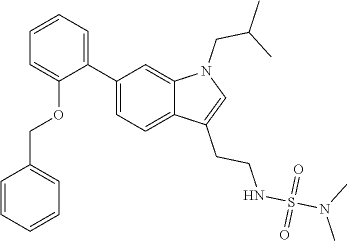 [SMILES: CC(C)Cn1cc(CCNS(=O)(=O)N(C)C)c2ccc(-c3ccccc3OCc3ccccc3)cc21] | B | D |
| 78 | 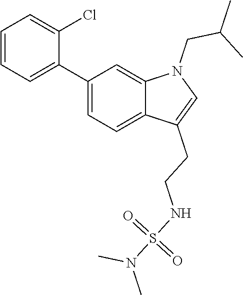 [SMILES: CC(C)Cn1cc(CCNS(=O)(=O)N(C)C)c2ccc(-c3ccccc3Cl)cc21] | A | C |
| 79 | 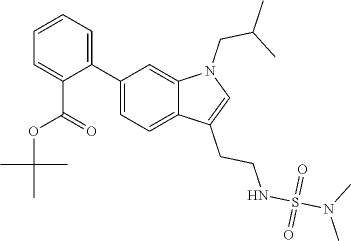 [SMILES: CC(C)Cn1cc(CCNS(=O)(=O)N(C)C)c2ccc(-c3ccccc3C(=O)OC(C)(C)C)cc21] | B | D |
| 80 | 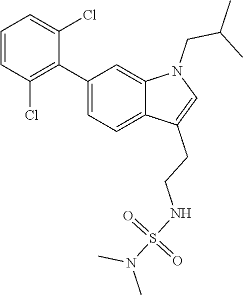 [SMILES: CC(C)Cn1cc(CCNS(=O)(=O)N(C)C)c2ccc(-c3c(Cl)cccc3Cl)cc21] | A | C |
| 81 | 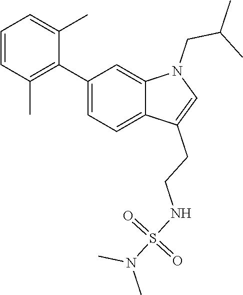 [SMILES: Cc1cccc(C)c1-c1ccc2c(CCNS(=O)(=O)N(C)C)cn(CC(C)C)c2c1] | A | D |
| 82 | 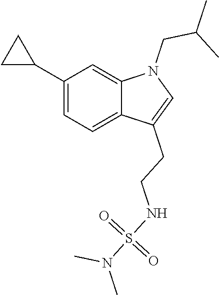 [SMILES: CC(C)Cn1cc(CCNS(=O)(=O)N(C)C)c2ccc(C3CC3)cc21] | A | C |
| 83 | 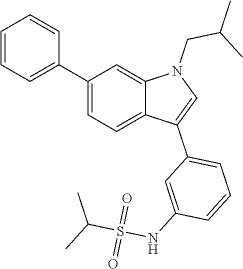 [SMILES: CC(C)Cn1cc(-c2cccc(NS(=O)(=O)C(C)C)c2)c2ccc(-c3ccccc3)cc21] | C | D |
| 84 | 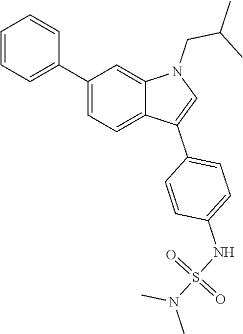 [SMILES: CC(C)Cn1cc(-c2ccc(NS(=O)(=O)N(C)C)cc2)c2ccc(-c3ccccc3)cc21] | C | D |
| 86 | 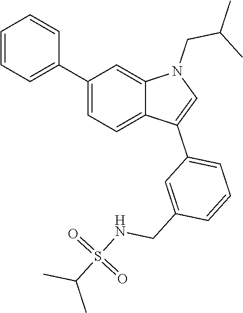 [SMILES: CC(C)Cn1cc(-c2cccc(CNS(=O)(=O)C(C)C)c2)c2ccc(-c3ccccc3)cc21] | B | D |
| 87 | 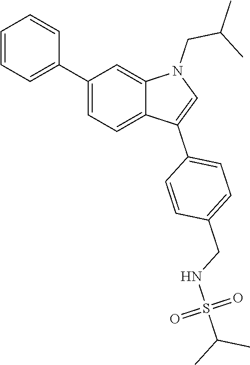 [SMILES: CC(C)Cn1cc(-c2ccc(CNS(=O)(=O)C(C)C)cc2)c2ccc(-c3ccccc3)cc21] | B | D |
| 88 | 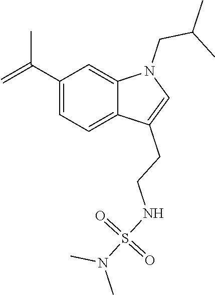 [SMILES: C=C(C)c1ccc2c(CCNS(=O)(=O)N(C)C)cn(CC(C)C)c2c1] | A | D |
| 91 | 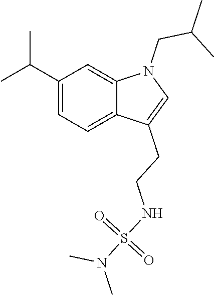 [SMILES: CC(C)Cn1cc(CCNS(=O)(=O)N(C)C)c2ccc(C(C)C)cc21] | A | B |
| 92 | 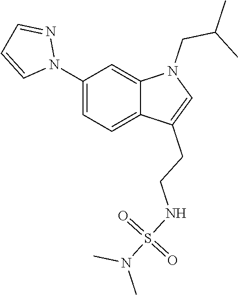 [SMILES: CC(C)Cn1cc(CCNS(=O)(=O)N(C)C)c2ccc(-n3cccn3)cc21] | B | D |
| 93 | 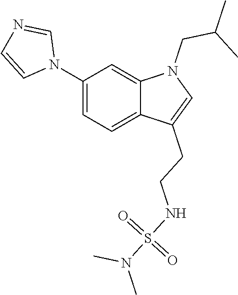 [SMILES: CC(C)Cn1cc(CCNS(=O)(=O)N(C)C)c2ccc(-n3ccnc3)cc21] | B | D |
| 95 | 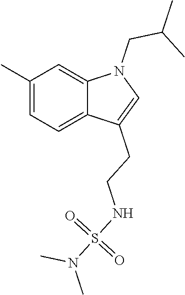 [SMILES: Cc1ccc2c(CCNS(=O)(=O)N(C)C)cn(CC(C)C)c2c1] | A | D |
| 96 | 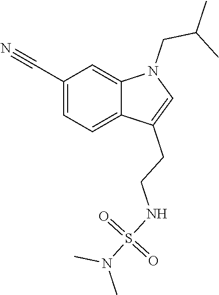 [SMILES: CC(C)Cn1cc(CCNS(=O)(=O)N(C)C)c2ccc(C#N)cc21] | A | D |
| 97 | 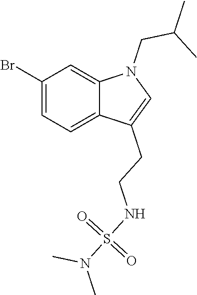 [SMILES: CC(C)Cn1cc(CCNS(=O)(=O)N(C)C)c2ccc(Br)cc21] | A | D |
| 98 | 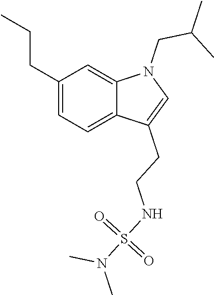 [SMILES: CCCc1ccc2c(CCNS(=O)(=O)N(C)C)cn(CC(C)C)c2c1] | A | C |
| 99 | 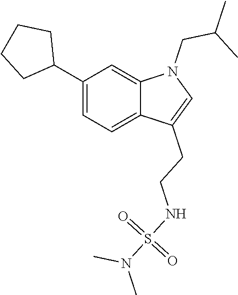 [SMILES: CC(C)Cn1cc(CCNS(=O)(=O)N(C)C)c2ccc(C3CCCC3)cc21] | A | B |
| 100 | 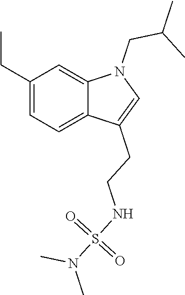 [SMILES: CCc1ccc2c(CCNS(=O)(=O)N(C)C)cn(CC(C)C)c2c1] | A | C |
| 101 | 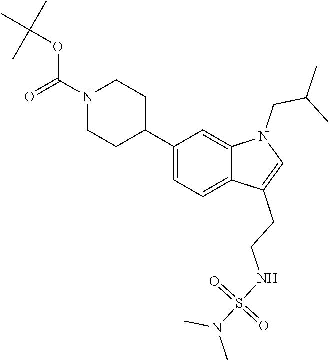 [SMILES: CC(C)Cn1cc(CCNS(=O)(=O)N(C)C)c2ccc(C3CCN(C(=O)OC(C)(C)C)CC3)cc21] | B | D |
| 102 | 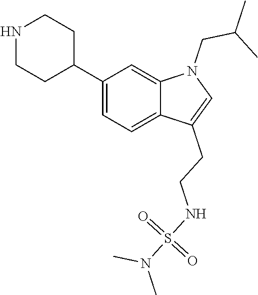 [SMILES: CC(C)Cn1cc(CCNS(=O)(=O)N(C)C)c2ccc(C3CCNCC3)cc21] | C | D |
| 103 | 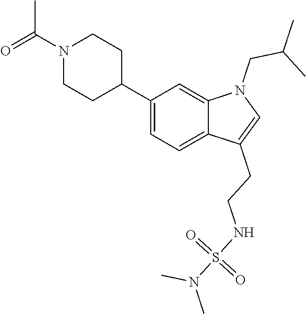 [SMILES: CC(=O)N1CCC(c2ccc3c(CCNS(=O)(=O)N(C)C)cn(CC(C)C)c3c2)CC1] | C | D |
| 105 | 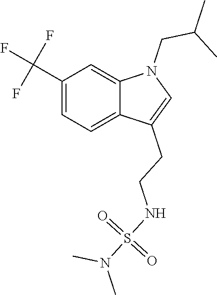 [SMILES: CC(C)Cn1cc(CCNS(=O)(=O)N(C)C)c2ccc(C(F)(F)F)cc21] | A | C |
| 106 | 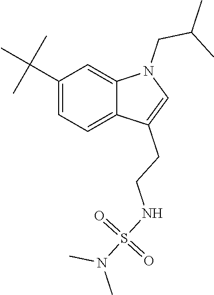 [SMILES: CC(C)Cn1cc(CCNS(=O)(=O)N(C)C)c2ccc(C(C)(C)C)cc21] | C | D |
| 107 | 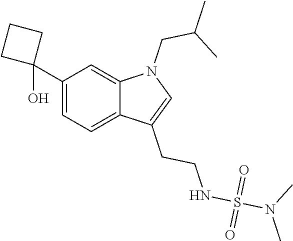 [SMILES: CC(C)Cn1cc(CCNS(=O)(=O)N(C)C)c2ccc(C3(O)CCC3)cc21] | B | D |
| 108 | 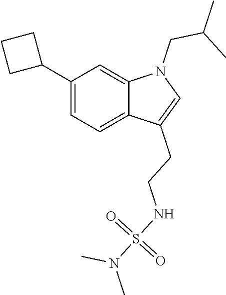 [SMILES: CC(C)Cn1cc(CCNS(=O)(=O)N(C)C)c2ccc(C3CCC3)cc21] | A | D |
| 109 | 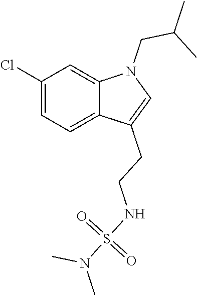 [SMILES: CC(C)Cn1cc(CCNS(=O)(=O)N(C)C)c2ccc(Cl)cc21] | A | D |
| 110 | 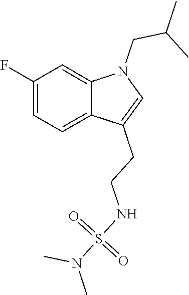 [SMILES: CC(C)Cn1cc(CCNS(=O)(=O)N(C)C)c2ccc(F)cc21] | B | D |
| 111 | 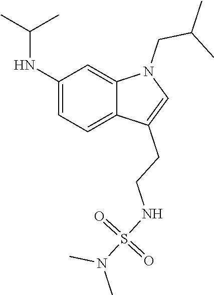 [SMILES: CC(C)Cn1cc(CCNS(=O)(=O)N(C)C)c2ccc(NC(C)C)cc21] | B | D |
| 112 | 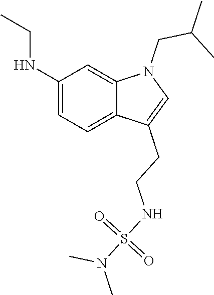 [SMILES: CCNc1ccc2c(CCNS(=O)(=O)N(C)C)cn(CC(C)C)c2c1] | C | D |
| 113 | 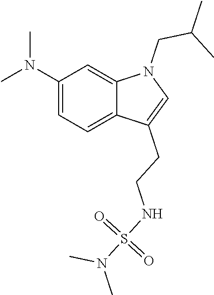 [SMILES: CC(C)Cn1cc(CCNS(=O)(=O)N(C)C)c2ccc(N(C)C)cc21] | B | D |
| 114 | 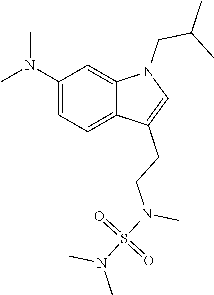 [SMILES: CC(C)Cn1cc(CCN(C)S(=O)(=O)N(C)C)c2ccc(N(C)C)cc21] | B | D |
| 115 | 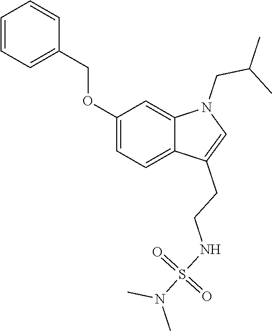 [SMILES: CC(C)Cn1cc(CCNS(=O)(=O)N(C)C)c2ccc(OCc3ccccc3)cc21] | B | D |
| 116 | 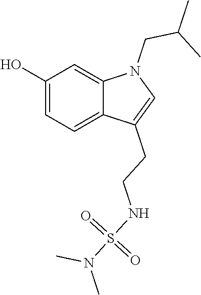 [SMILES: CC(C)Cn1cc(CCNS(=O)(=O)N(C)C)c2ccc(O)cc21] | C | D |
| 117 | 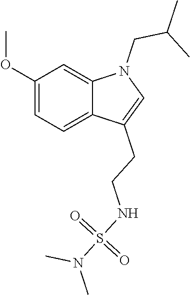 [SMILES: COc1ccc2c(CCNS(=O)(=O)N(C)C)cn(CC(C)C)c2c1] | C | D |
| 118 | 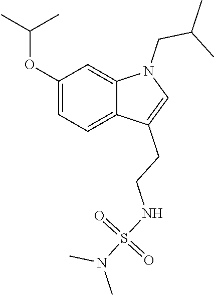 [SMILES: CC(C)Cn1cc(CCNS(=O)(=O)N(C)C)c2ccc(OC(C)C)cc21] | B | D |
| 119 | 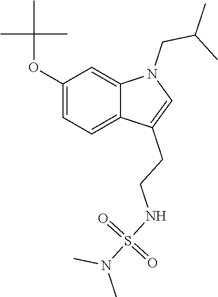 [SMILES: CC(C)Cn1cc(CCNS(=O)(=O)N(C)C)c2ccc(OC(C)(C)C)cc21] | B | D |
| 120 | 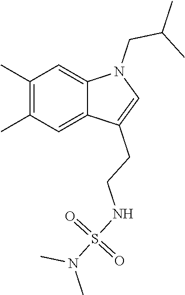 [SMILES: Cc1cc2c(CCNS(=O)(=O)N(C)C)cn(CC(C)C)c2cc1C] | A | C |
| 121 | 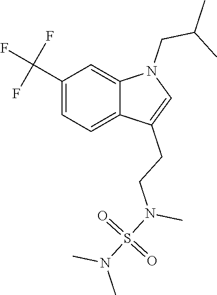 [SMILES: CC(C)Cn1cc(CCN(C)S(=O)(=O)N(C)C)c2ccc(C(F)(F)F)cc21] | B | D |
| 122 | 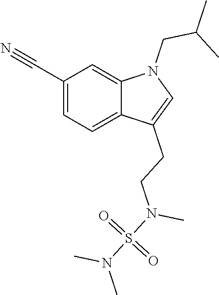 [SMILES: CC(C)Cn1cc(CCN(C)S(=O)(=O)N(C)C)c2ccc(C#N)cc21] | C | D |
| 123 | 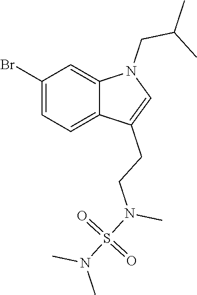 [SMILES: CC(C)Cn1cc(CCN(C)S(=O)(=O)N(C)C)c2ccc(Br)cc21] | B | D |
| 124 | 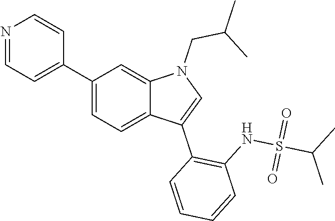 [SMILES: CC(C)Cn1cc(-c2ccccc2NS(=O)(=O)C(C)C)c2ccc(-c3ccncc3)cc21] | C | D |
| 125 | 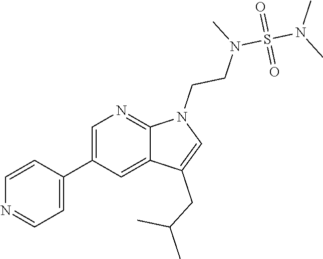 [SMILES: CC(C)Cc1cn(CCN(C)S(=O)(=O)N(C)C)c2ncc(-c3ccncc3)cc12] | A | D |
| 126 | 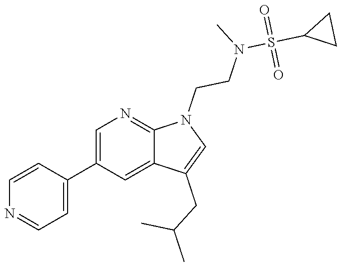 [SMILES: CC(C)Cc1cn(CCN(C)S(=O)(=O)C2CC2)c2ncc(-c3ccncc3)cc12] | B | D |
| 127 | 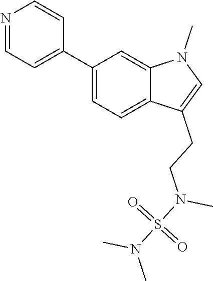 [SMILES: CN(C)S(=O)(=O)N(C)CCc1cn(C)c2cc(-c3ccncc3)ccc12] | B | D |
| 128 | 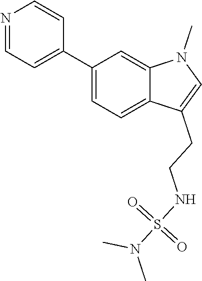 [SMILES: CN(C)S(=O)(=O)NCCc1cn(C)c2cc(-c3ccncc3)ccc12] | B | D |
| 129 | 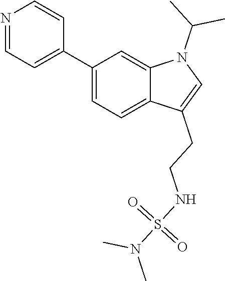 [SMILES: CC(C)n1cc(CCNS(=O)(=O)N(C)C)c2ccc(-c3ccncc3)cc21] | B | D |
| 130 | 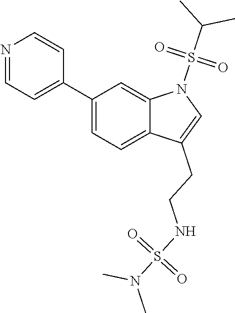 [SMILES: CC(C)S(=O)(=O)n1cc(CCNS(=O)(=O)N(C)C)c2ccc(-c3ccncc3)cc21] | C | D |
| 132 | 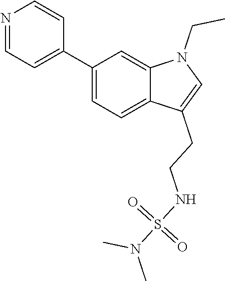 [SMILES: CCn1cc(CCNS(=O)(=O)N(C)C)c2ccc(-c3ccncc3)cc21] | C | D |
| 133 | 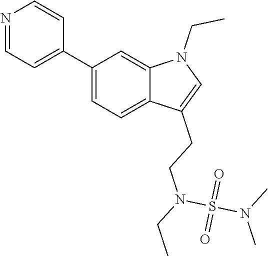 [SMILES: CCN(CCc1cn(CC)c2cc(-c3ccncc3)ccc12)S(=O)(=O)N(C)C] | B | D |
| 134 | 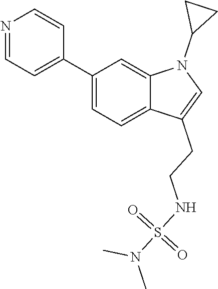 [SMILES: CN(C)S(=O)(=O)NCCc1cn(C2CC2)c2cc(-c3ccncc3)ccc12] | C | D |
| 135 | 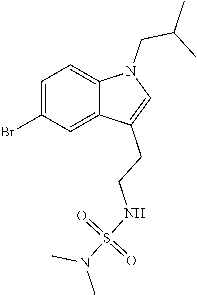 [SMILES: CC(C)Cn1cc(CCNS(=O)(=O)N(C)C)c2cc(Br)ccc21] | A | D |
| 136 | 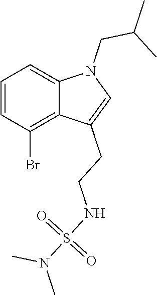 [SMILES: CC(C)Cn1cc(CCNS(=O)(=O)N(C)C)c2c(Br)cccc21] | B | D |
| 137 | 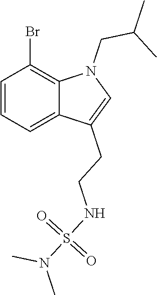 [SMILES: CC(C)Cn1cc(CCNS(=O)(=O)N(C)C)c2cccc(Br)c21] | B | C |
| 140 | 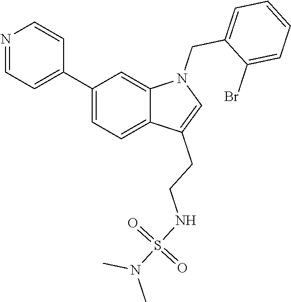 [SMILES: CN(C)S(=O)(=O)NCCc1cn(Cc2ccccc2Br)c2cc(-c3ccncc3)ccc12] | C | D |
| 141 | 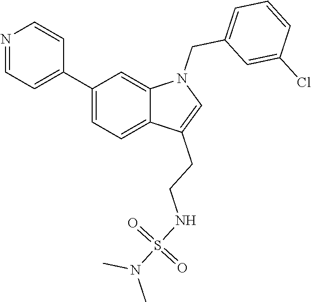 [SMILES: CN(C)S(=O)(=O)NCCc1cn(Cc2cccc(Cl)c2)c2cc(-c3ccncc3)ccc12] | A | D |
| 142 | 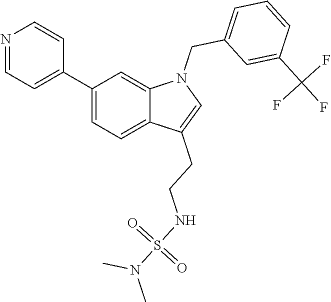 [SMILES: CN(C)S(=O)(=O)NCCc1cn(Cc2cccc(C(F)(F)F)c2)c2cc(-c3ccncc3)ccc12] | B | D |
| 143 | 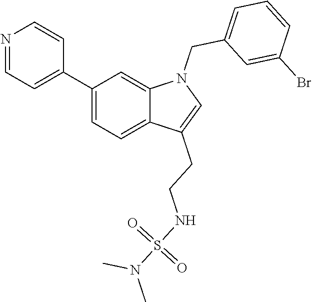 [SMILES: CN(C)S(=O)(=O)NCCc1cn(Cc2cccc(Br)c2)c2cc(-c3ccncc3)ccc12] | A | D |
| 144 | 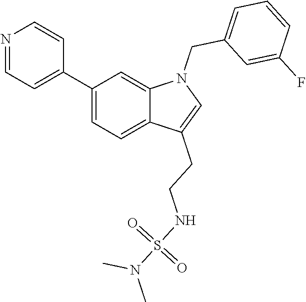 [SMILES: CN(C)S(=O)(=O)NCCc1cn(Cc2cccc(F)c2)c2cc(-c3ccncc3)ccc12] | A | D |
| 145 | 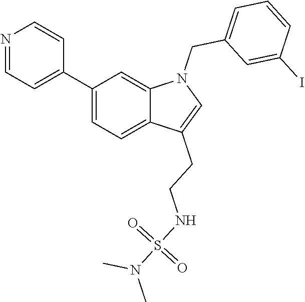 [SMILES: CN(C)S(=O)(=O)NCCc1cn(Cc2cccc(I)c2)c2cc(-c3ccncc3)ccc12] | C | D |
| 146 | 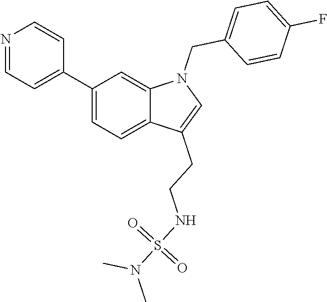 [SMILES: CN(C)S(=O)(=O)NCCc1cn(Cc2ccc(F)cc2)c2cc(-c3ccncc3)ccc12] | B | D |
| 147 | 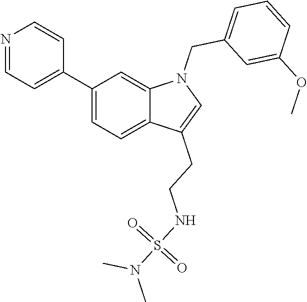 [SMILES: COc1cccc(Cn2cc(CCNS(=O)(=O)N(C)C)c3ccc(-c4ccncc4)cc32)c1] | B | D |
| 148 | 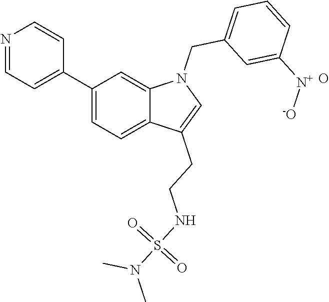 | B | D |
| 149 | 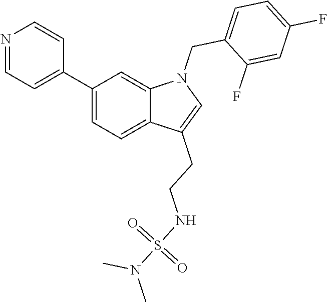 [SMILES: CN(C)S(=O)(=O)NCCc1cn(Cc2ccc(F)cc2F)c2cc(-c3ccncc3)ccc12] | B | D |
| 150 | 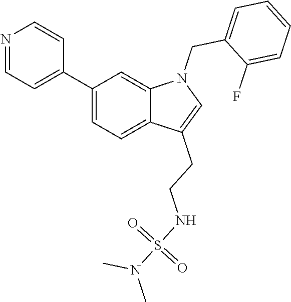 [SMILES: CN(C)S(=O)(=O)NCCc1cn(Cc2ccccc2F)c2cc(-c3ccncc3)ccc12] | B | D |
| 151 | 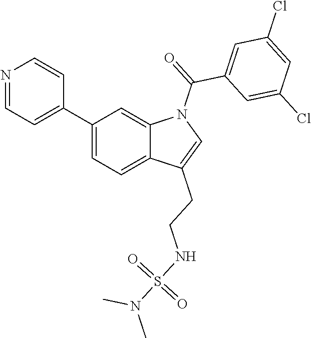 [SMILES: CN(C)S(=O)(=O)NCCc1cn(C(=O)c2cc(Cl)cc(Cl)c2)c2cc(-c3ccncc3)ccc12] | C | D |
| 152 | 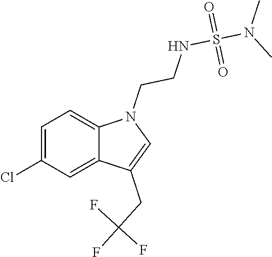 [SMILES: CN(C)S(=O)(=O)NCCn1cc(CC(F)(F)F)c2cc(Cl)ccc21] | B | D |
| 153 | 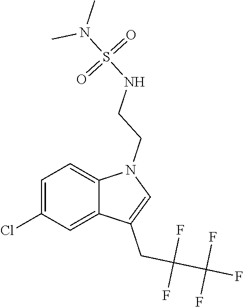 [SMILES: CN(C)S(=O)(=O)NCCn1cc(CC(F)(F)C(F)(F)F)c2cc(Cl)ccc21] | B | D |
| 154 | 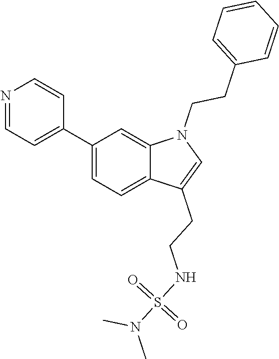 [SMILES: CN(C)S(=O)(=O)NCCc1cn(CCc2ccccc2)c2cc(-c3ccncc3)ccc12] | B | D |
| 155 | 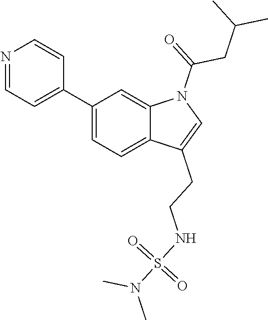 [SMILES: CC(C)CC(=O)n1cc(CCNS(=O)(=O)N(C)C)c2ccc(-c3ccncc3)cc21] | B | D |
| 156 | 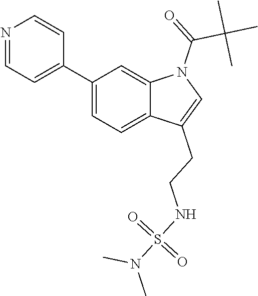 [SMILES: CN(C)S(=O)(=O)NCCc1cn(C(=O)C(C)(C)C)c2cc(-c3ccncc3)ccc12] | C | D |
| 157 | 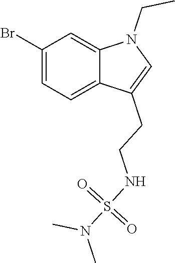 [SMILES: CCn1cc(CCNS(=O)(=O)N(C)C)c2ccc(Br)cc21] | B | D |
| 158 | 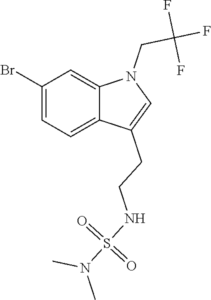 [SMILES: CN(C)S(=O)(=O)NCCc1cn(CC(F)(F)F)c2cc(Br)ccc12] | B | D |
| 159 | 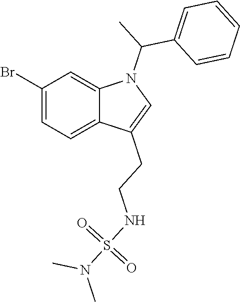 [SMILES: CC(c1ccccc1)n1cc(CCNS(=O)(=O)N(C)C)c2ccc(Br)cc21] | B | D |
| 160 | 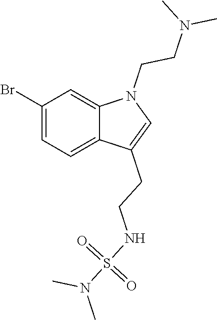 [SMILES: CN(C)CCn1cc(CCNS(=O)(=O)N(C)C)c2ccc(Br)cc21] | C | D |
| 161 | 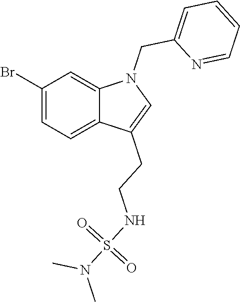 [SMILES: CN(C)S(=O)(=O)NCCc1cn(Cc2ccccn2)c2cc(Br)ccc12] | B | D |
| 162 | 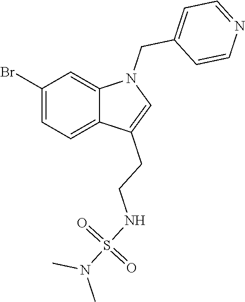 [SMILES: CN(C)S(=O)(=O)NCCc1cn(Cc2ccncc2)c2cc(Br)ccc12] | B | D |
| 163 | 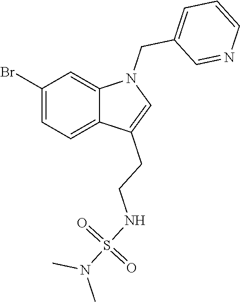 [SMILES: CN(C)S(=O)(=O)NCCc1cn(Cc2cccnc2)c2cc(Br)ccc12] | B | D |
| 164 | 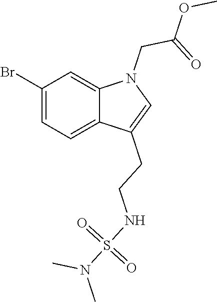 [SMILES: COC(=O)Cn1cc(CCNS(=O)(=O)N(C)C)c2ccc(Br)cc21] | C | D |
| 165 | 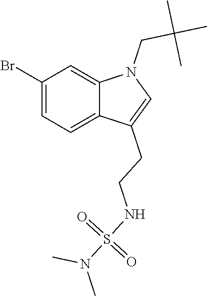 [SMILES: CN(C)S(=O)(=O)NCCc1cn(CC(C)(C)C)c2cc(Br)ccc12] | B | D |
| 166 | 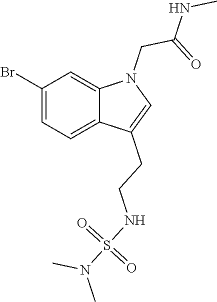 [SMILES: CNC(=O)Cn1cc(CCNS(=O)(=O)N(C)C)c2ccc(Br)cc21] | B | D |
| 167 | 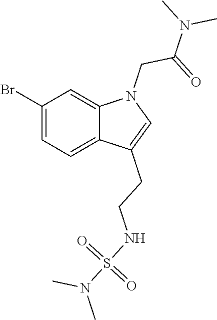 [SMILES: CN(C)C(=O)Cn1cc(CCNS(=O)(=O)N(C)C)c2ccc(Br)cc21] | B | D |
| 168 | 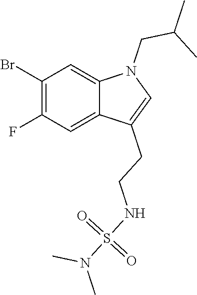 [SMILES: CC(C)Cn1cc(CCNS(=O)(=O)N(C)C)c2cc(F)c(Br)cc21] | A | D |
| 169 | 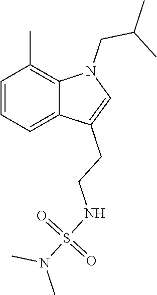 [SMILES: Cc1cccc2c(CCNS(=O)(=O)N(C)C)cn(CC(C)C)c12] | B | D |
| 170 | 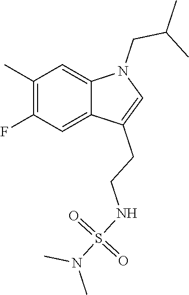 [SMILES: Cc1cc2c(cc1F)c(CCNS(=O)(=O)N(C)C)cn2CC(C)C] | A | D |
| 171 | 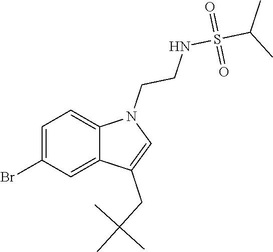 [SMILES: CC(C)S(=O)(=O)NCCn1cc(CC(C)(C)C)c2cc(Br)ccc21] | B | D |
| 172 | 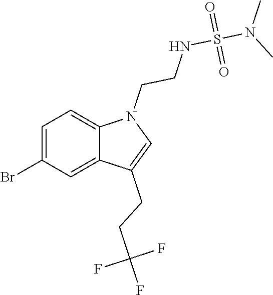 [SMILES: CN(C)S(=O)(=O)NCCn1cc(CCC(F)(F)F)c2cc(Br)ccc21] | B | D |
| 173 | 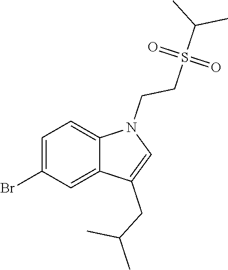 [SMILES: CC(C)Cc1cn(CCS(=O)(=O)C(C)C)c2ccc(Br)cc12] | B | D |
| 174 | 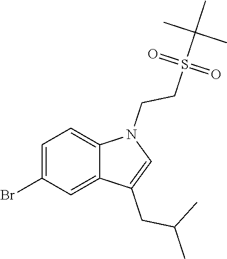 [SMILES: CC(C)Cc1cn(CCS(=O)(=O)C(C)(C)C)c2ccc(Br)cc12] | B | D |
| 175 | 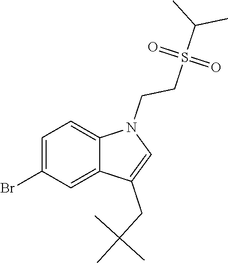 [SMILES: CC(C)S(=O)(=O)CCn1cc(CC(C)(C)C)c2cc(Br)ccc21] | C | D |
| 176 | 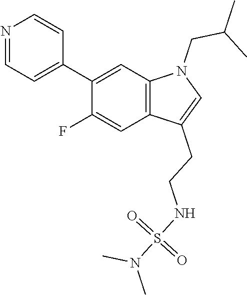 | A | D |
| 177 | 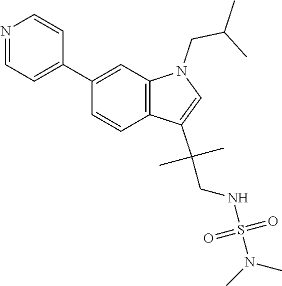 [SMILES: CC(C)Cn1cc(C(C)(C)CNS(=O)(=O)N(C)C)c2ccc(-c3ccncc3)cc21] | B | D |
| 178 | 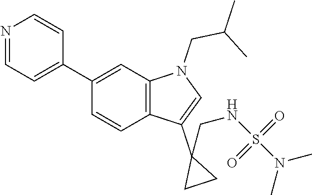 [SMILES: CC(C)Cn1cc(C2(CNS(=O)(=O)N(C)C)CC2)c2ccc(-c3ccncc3)cc21] | A | D |
| 179 | 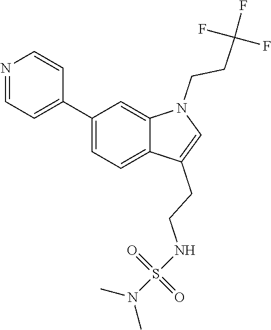 [SMILES: CN(C)S(=O)(=O)NCCc1cn(CCC(F)(F)F)c2cc(-c3ccncc3)ccc12] | A | D |
| 180 | 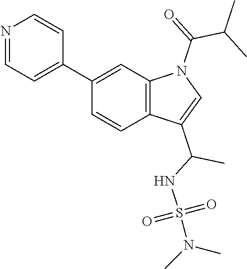 [SMILES: CC(C)C(=O)n1cc(C(C)NS(=O)(=O)N(C)C)c2ccc(-c3ccncc3)cc21] | A | C |
| 181 | 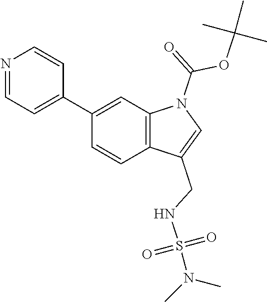 [SMILES: CN(C)S(=O)(=O)NCc1cn(C(=O)OC(C)(C)C)c2cc(-c3ccncc3)ccc12] | B | D |
| 182 | 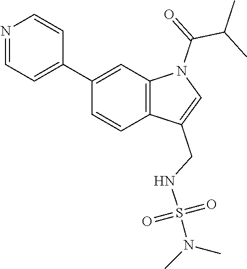 [SMILES: CC(C)C(=O)n1cc(CNS(=O)(=O)N(C)C)c2ccc(-c3ccncc3)cc21] | B | D |
| 183 | 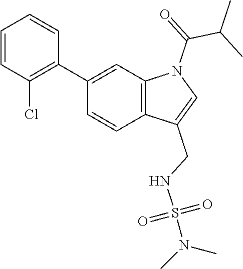 [SMILES: CC(C)C(=O)n1cc(CNS(=O)(=O)N(C)C)c2ccc(-c3ccccc3Cl)cc21] | B | D |
| 184 | 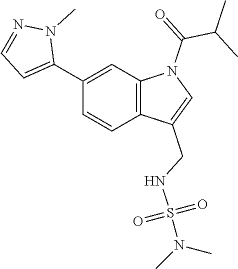 [SMILES: CC(C)C(=O)n1cc(CNS(=O)(=O)N(C)C)c2ccc(-c3ccnn3C)cc21] | C | D |
| 185 | 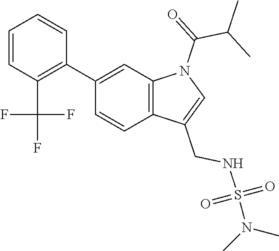 [SMILES: CC(C)C(=O)n1cc(CNS(=O)(=O)N(C)C)c2ccc(-c3ccccc3C(F)(F)F)cc21] | A | D |
| 186 | 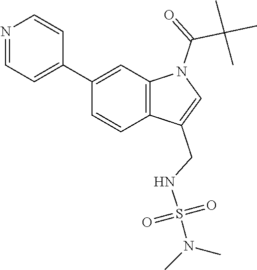 [SMILES: CN(C)S(=O)(=O)NCc1cn(C(=O)C(C)(C)C)c2cc(-c3ccncc3)ccc12] | B | D |
| 187 | 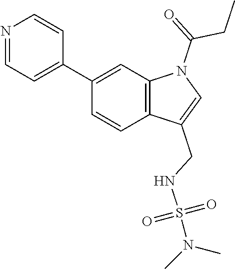 [SMILES: CCC(=O)n1cc(CNS(=O)(=O)N(C)C)c2ccc(-c3ccncc3)cc21] | C | D |
| 188 | 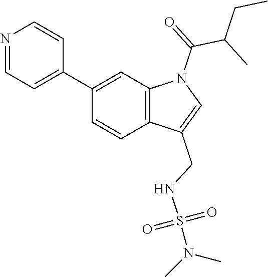 [SMILES: CCC(C)C(=O)n1cc(CNS(=O)(=O)N(C)C)c2ccc(-c3ccncc3)cc21] | A | V |
| 189 | 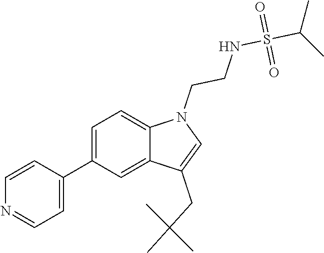 [SMILES: CC(C)S(=O)(=O)NCCn1cc(CC(C)(C)C)c2cc(-c3ccncc3)ccc21] | A | D |
| 190 | 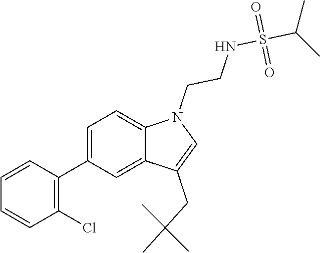 [SMILES: CC(C)S(=O)(=O)NCCn1cc(CC(C)(C)C)c2cc(-c3ccccc3Cl)ccc21] | A | D |
| 191 | 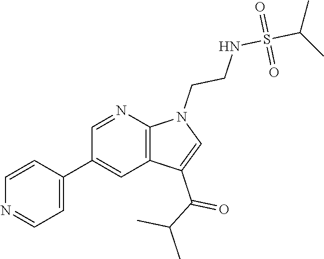 [SMILES: CC(C)C(=O)c1cn(CCNS(=O)(=O)C(C)C)c2ncc(-c3ccncc3)cc12] | C | D |
| 192 | 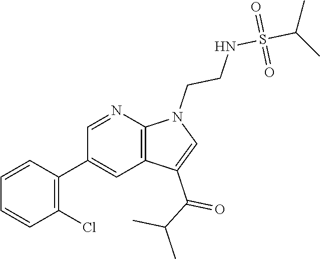 [SMILES: CC(C)C(=O)c1cn(CCNS(=O)(=O)C(C)C)c2ncc(-c3ccccc3Cl)cc12] | B | D |
| 194 | 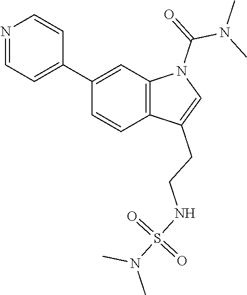 | C | D |
| 195 | 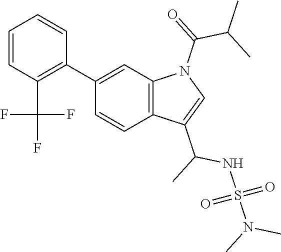 [SMILES: CC(C)C(=O)n1cc(C(C)NS(=O)(=O)N(C)C)c2ccc(-c3ccccc3C(F)(F)F)cc21] | A | B |
| 196 | 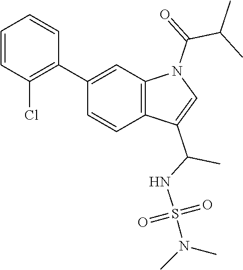 [SMILES: CC(C)C(=O)n1cc(C(C)NS(=O)(=O)N(C)C)c2ccc(-c3ccccc3Cl)cc21] | A | D |
| 197 | 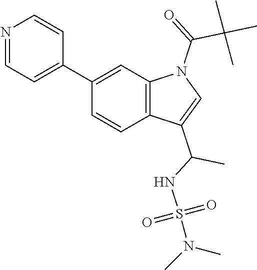 [SMILES: CC(NS(=O)(=O)N(C)C)c1cn(C(=O)C(C)(C)C)c2cc(-c3ccncc3)ccc12] | A | D |
| 199 | 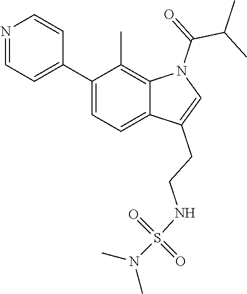 [SMILES: Cc1c(-c2ccncc2)ccc2c(CCNS(=O)(=O)N(C)C)cn(C(=O)C(C)C)c12] | A | B |
| 200 | 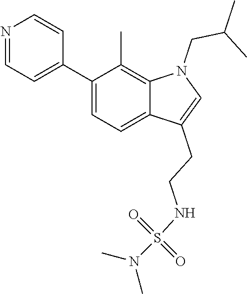 [SMILES: Cc1c(-c2ccncc2)ccc2c(CCNS(=O)(=O)N(C)C)cn(CC(C)C)c12] | A | C |
| 202 | 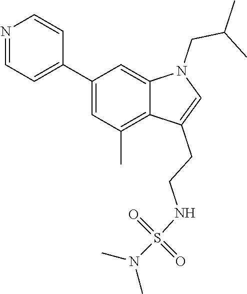 [SMILES: Cc1cc(-c2ccncc2)cc2c1c(CCNS(=O)(=O)N(C)C)cn2CC(C)C] | C | D |
| 203 | 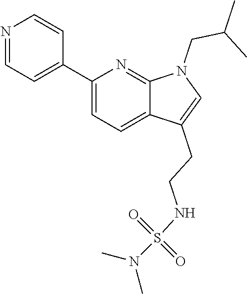 [SMILES: CC(C)Cn1cc(CCNS(=O)(=O)N(C)C)c2ccc(-c3ccncc3)nc21] | A | C |
| 204 | 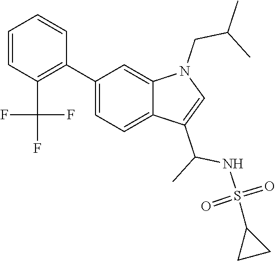 [SMILES: CC(C)Cn1cc(C(C)NS(=O)(=O)C2CC2)c2ccc(-c3ccccc3C(F)(F)F)cc21] | A | D |
| 205 | 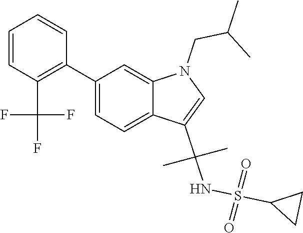 [SMILES: CC(C)Cn1cc(C(C)(C)NS(=O)(=O)C2CC2)c2ccc(-c3ccccc3C(F)(F)F)cc21] | A | D |
| 206 | 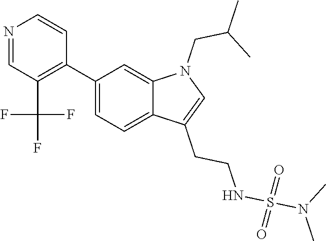 [SMILES: CC(C)Cn1cc(CCNS(=O)(=O)N(C)C)c2ccc(-c3ccncc3C(F)(F)F)cc21] | A | D |
| 207 | 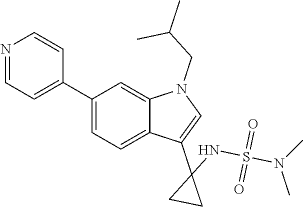 [SMILES: CC(C)Cn1cc(C2(NS(=O)(=O)N(C)C)CC2)c2ccc(-c3ccncc3)cc21] | C | D |
| 208 | 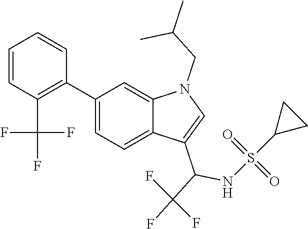 [SMILES: CC(C)Cn1cc(C(NS(=O)(=O)C2CC2)C(F)(F)F)c2ccc(-c3ccccc3C(F)(F)F)cc21] | A | D |
| 209 | 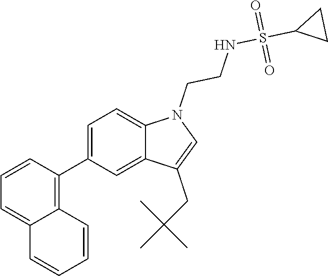 [SMILES: CC(C)(C)Cc1cn(CCNS(=O)(=O)C2CC2)c2ccc(-c3cccc4ccccc34)cc12] | A | D |
| 210 | 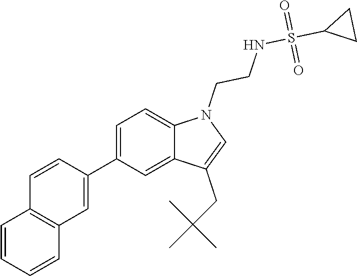 [SMILES: CC(C)(C)Cc1cn(CCNS(=O)(=O)C2CC2)c2ccc(-c3ccc4ccccc4c3)cc12] | B | D |
| 211 | 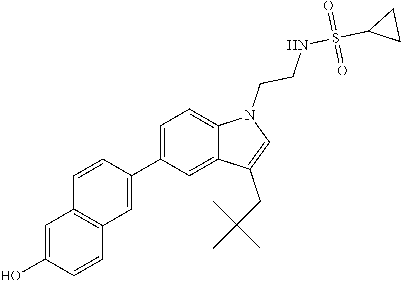 [SMILES: CC(C)(C)Cc1cn(CCNS(=O)(=O)C2CC2)c2ccc(-c3ccc4cc(O)ccc4c3)cc12] | B | D |
| 212 | 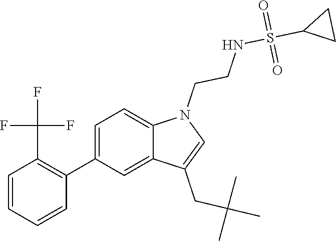 [SMILES: CC(C)(C)Cc1cn(CCNS(=O)(=O)C2CC2)c2ccc(-c3ccccc3C(F)(F)F)cc12] | A | B |
| 213 | 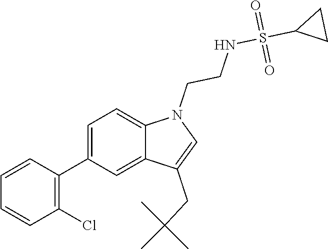 [SMILES: CC(C)(C)Cc1cn(CCNS(=O)(=O)C2CC2)c2ccc(-c3ccccc3Cl)cc12] | A | D |
| 214 | 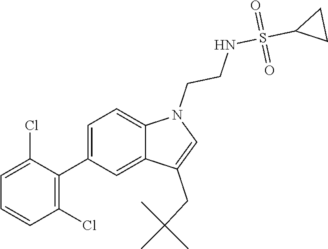 [SMILES: CC(C)(C)Cc1cn(CCNS(=O)(=O)C2CC2)c2ccc(-c3c(Cl)cccc3Cl)cc12] | A | B |
| 215 | 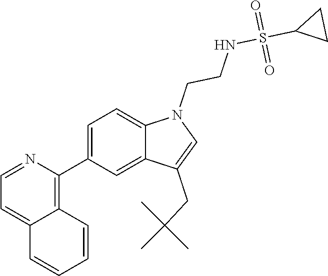 [SMILES: CC(C)(C)Cc1cn(CCNS(=O)(=O)C2CC2)c2ccc(-c3nccc4ccccc34)cc12] | C | D |
| 216 | 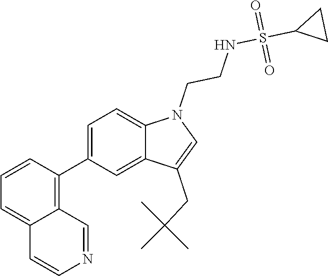 [SMILES: CC(C)(C)Cc1cn(CCNS(=O)(=O)C2CC2)c2ccc(-c3cccc4ccncc34)cc12] | B | C |
| 217 | 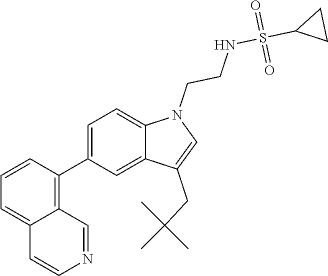 [SMILES: CC(C)(C)Cc1cn(CCNS(=O)(=O)C2CC2)c2ccc(-c3cccc4ccncc34)cc12] | B | D |
| 218 | 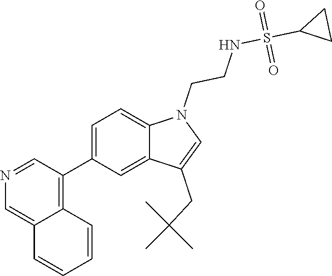 [SMILES: CC(C)(C)Cc1cn(CCNS(=O)(=O)C2CC2)c2ccc(-c3cncc4ccccc34)cc12] | B | D |
| 219 | 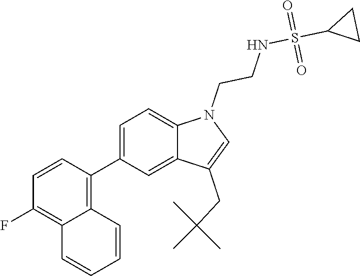 [SMILES: CC(C)(C)Cc1cn(CCNS(=O)(=O)C2CC2)c2ccc(-c3ccc(F)c4ccccc34)cc12] | A | D |
| 220 | 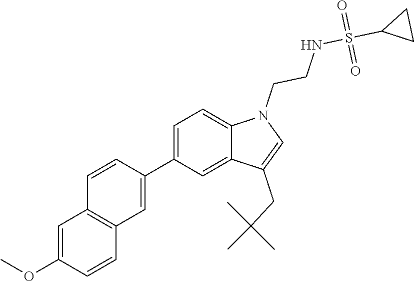 [SMILES: COc1ccc2cc(-c3ccc4c(c3)c(CC(C)(C)C)cn4CCNS(=O)(=O)C3CC3)ccc2c1] | C | D |
| 221 | 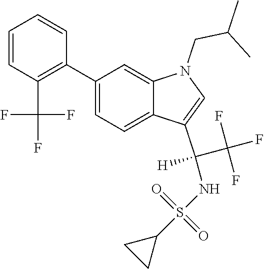 [SMILES: CC(C)Cn1cc([C@H](NS(=O)(=O)C2CC2)C(F)(F)F)c2ccc(-c3ccccc3C(F)(F)F)cc21] | A | D |
| 222 | 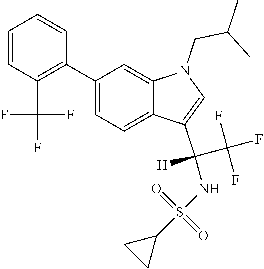 [SMILES: CC(C)Cn1cc([C@@H](NS(=O)(=O)C2CC2)C(F)(F)F)c2ccc(-c3ccccc3C(F)(F)F)cc21] | B | D |
| 223 | 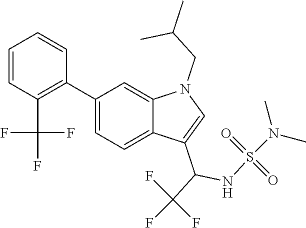 [SMILES: CC(C)Cn1cc(C(NS(=O)(=O)N(C)C)C(F)(F)F)c2ccc(-c3ccccc3C(F)(F)F)cc21] | A | D |
| 224 | 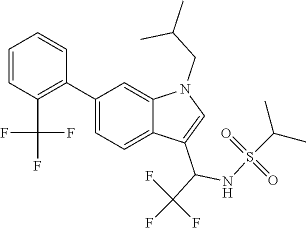 [SMILES: CC(C)Cn1cc(C(NS(=O)(=O)C(C)C)C(F)(F)F)c2ccc(-c3ccccc3C(F)(F)F)cc21] | B | D |
| 225 | 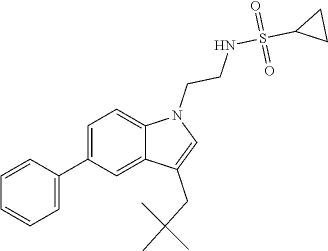 [SMILES: CC(C)(C)Cc1cn(CCNS(=O)(=O)C2CC2)c2ccc(-c3ccccc3)cc12] | B | D |
| 226 | 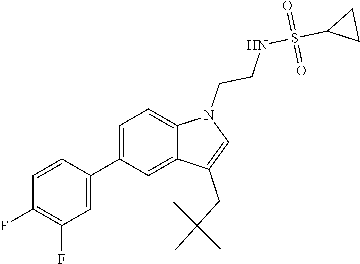 [SMILES: CC(C)(C)Cc1cn(CCNS(=O)(=O)C2CC2)c2ccc(-c3ccc(F)c(F)c3)cc12] | B | D |
| 227 | 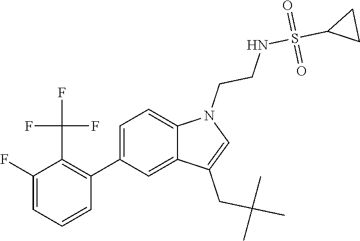 [SMILES: CC(C)(C)Cc1cn(CCNS(=O)(=O)C2CC2)c2ccc(-c3cccc(F)c3C(F)(F)F)cc12] | A | B |
| 228 | 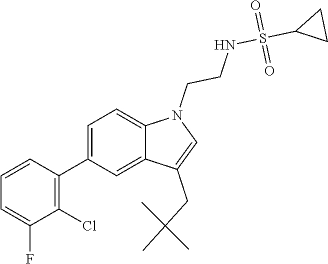 [SMILES: CC(C)(C)Cc1cn(CCNS(=O)(=O)C2CC2)c2ccc(-c3cccc(F)c3Cl)cc12] | A | D |
| 229 | 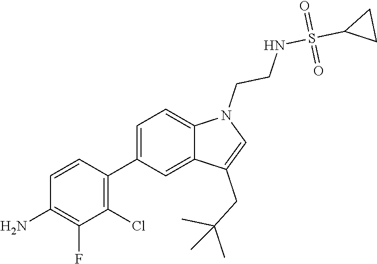 [SMILES: CC(C)(C)Cc1cn(CCNS(=O)(=O)C2CC2)c2ccc(-c3ccc(N)c(F)c3Cl)cc12] | A | D |
| 230 | 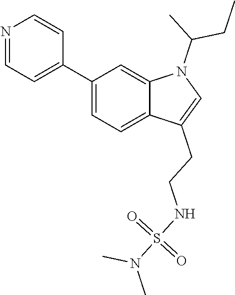 [SMILES: CCC(C)n1cc(CCNS(=O)(=O)N(C)C)c2ccc(-c3ccncc3)cc21] | A | B |
| 231 | 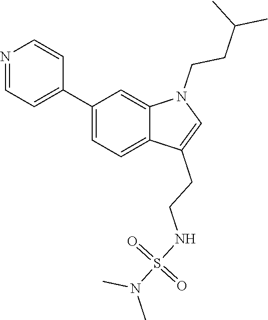 [SMILES: CC(C)CCn1cc(CCNS(=O)(=O)N(C)C)c2ccc(-c3ccncc3)cc21] | A | B |
| 232 | 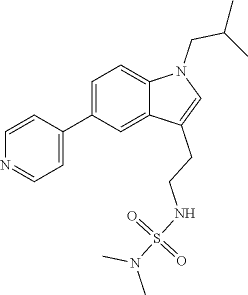 | C | D |
| 233 | 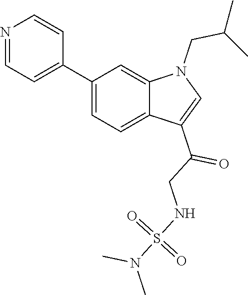 | C | D |
| 234 | 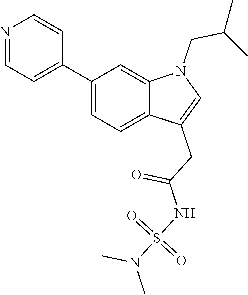 | C | D |
| 235 | 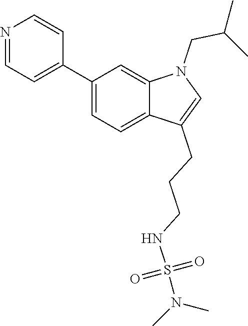 [SMILES: CC(C)Cn1cc(CCCNS(=O)(=O)N(C)C)c2ccc(-c3ccncc3)cc21] | C | D |
| 236 | 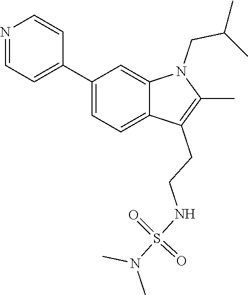 [SMILES: Cc1c(CCNS(=O)(=O)N(C)C)c2ccc(-c3ccncc3)cc2n1CC(C)C] | B | D |
| 237 | 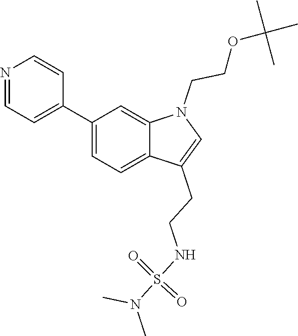 [SMILES: CN(C)S(=O)(=O)NCCc1cn(CCOC(C)(C)C)c2cc(-c3ccncc3)ccc12] | B | D |
| 238 | 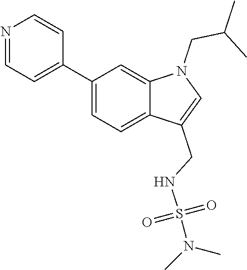 [SMILES: CC(C)Cn1cc(CNS(=O)(=O)N(C)C)c2ccc(-c3ccncc3)cc21] | B | D |
| 239 | 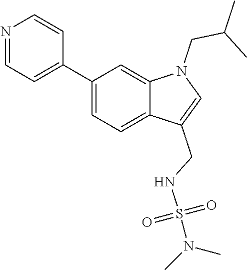 [SMILES: CC(C)Cn1cc(CNS(=O)(=O)N(C)C)c2ccc(-c3ccncc3)cc21] | A | D |
| 242 | 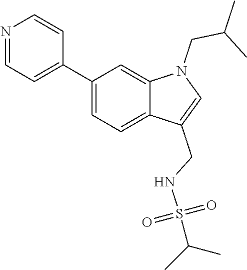 [SMILES: CC(C)Cn1cc(CNS(=O)(=O)C(C)C)c2ccc(-c3ccncc3)cc21] | A | D |
| 243 | 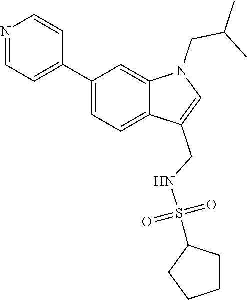 [SMILES: CC(C)Cn1cc(CNS(=O)(=O)C2CCCC2)c2ccc(-c3ccncc3)cc21] | A | D |
| 244 | 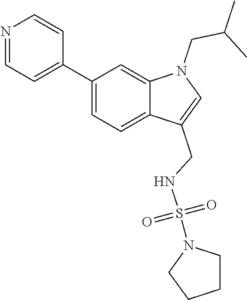 [SMILES: CC(C)Cn1cc(CNS(=O)(=O)N2CCCC2)c2ccc(-c3ccncc3)cc21] | A | D |
| 245 | 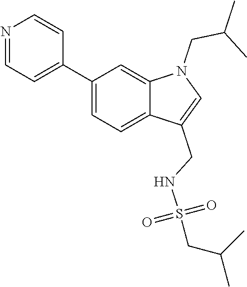 [SMILES: CC(C)Cn1cc(CNS(=O)(=O)CC(C)C)c2ccc(-c3ccncc3)cc21] | A | D |
| 246 | 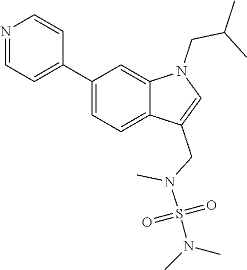 [SMILES: CC(C)Cn1cc(CN(C)S(=O)(=O)N(C)C)c2ccc(-c3ccncc3)cc21] | B | D |
| 247 | 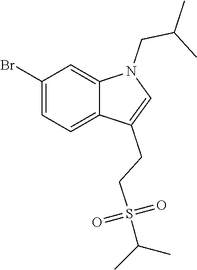 [SMILES: CC(C)Cn1cc(CCS(=O)(=O)C(C)C)c2ccc(Br)cc21] | B | D |
| 248 | 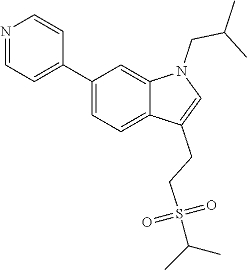 [SMILES: CC(C)Cn1cc(CCS(=O)(=O)C(C)C)c2ccc(-c3ccncc3)cc21] | A | D |
| 249 | 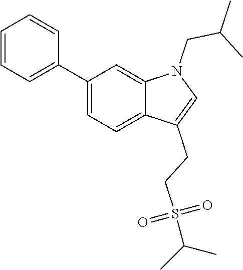 [SMILES: CC(C)Cn1cc(CCS(=O)(=O)C(C)C)c2ccc(-c3ccccc3)cc21] | A | D |
| 250 | 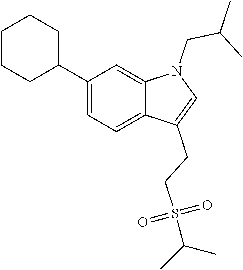 [SMILES: CC(C)Cn1cc(CCS(=O)(=O)C(C)C)c2ccc(C3CCCCC3)cc21] | A | D |
| 251 | 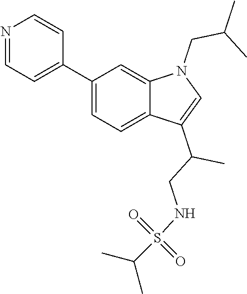 [SMILES: CC(C)Cn1cc(C(C)CNS(=O)(=O)C(C)C)c2ccc(-c3ccncc3)cc21] | A | D |
| 252 | 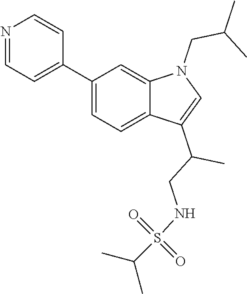 [SMILES: CC(C)Cn1cc(C(C)CNS(=O)(=O)C(C)C)c2ccc(-c3ccncc3)cc21] | B | D |
| 253 | 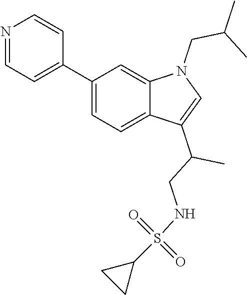 [SMILES: CC(C)Cn1cc(C(C)CNS(=O)(=O)C2CC2)c2ccc(-c3ccncc3)cc21] | B | D |
| 254 | 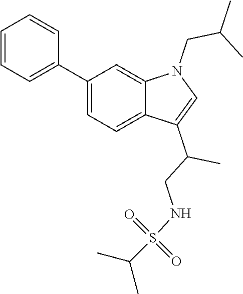 [SMILES: CC(C)Cn1cc(C(C)CNS(=O)(=O)C(C)C)c2ccc(-c3ccccc3)cc21] | C | D |
| 255 | 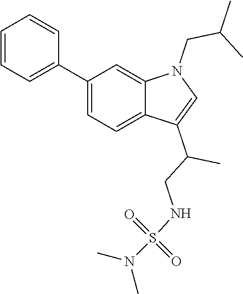 [SMILES: CC(C)Cn1cc(C(C)CNS(=O)(=O)N(C)C)c2ccc(-c3ccccc3)cc21] | B | D |
| 256 | 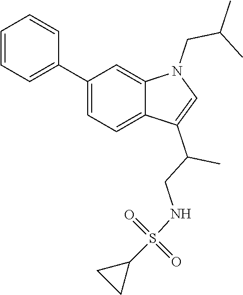 [SMILES: CC(C)Cn1cc(C(C)CNS(=O)(=O)C2CC2)c2ccc(-c3ccccc3)cc21] | C | D |
| 257 | 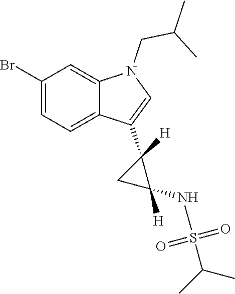 [SMILES: CC(C)Cn1cc([C@H]2C[C@H]2NS(=O)(=O)C(C)C)c2ccc(Br)cc21] | B | D |
| 258 | 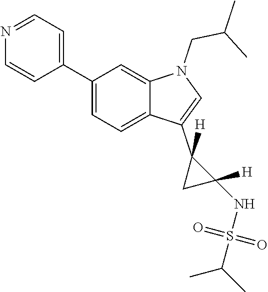 [SMILES: CC(C)Cn1cc([C@H]2C[C@H]2NS(=O)(=O)C(C)C)c2ccc(-c3ccncc3)cc21] | A | D |
| 259 | 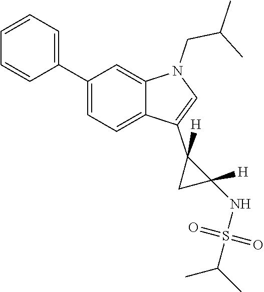 [SMILES: CC(C)Cn1cc([C@H]2C[C@H]2NS(=O)(=O)C(C)C)c2ccc(-c3ccccc3)cc21] | C | D |
| 260 | 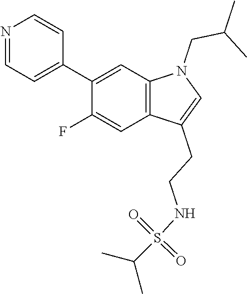 [SMILES: CC(C)Cn1cc(CCNS(=O)(=O)C(C)C)c2cc(F)c(-c3ccncc3)cc21] | A | D |
| 261 | 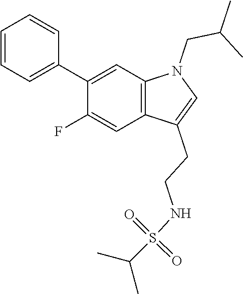 [SMILES: CC(C)Cn1cc(CCNS(=O)(=O)C(C)C)c2cc(F)c(-c3ccccc3)cc21] | A | D |
| 262 | 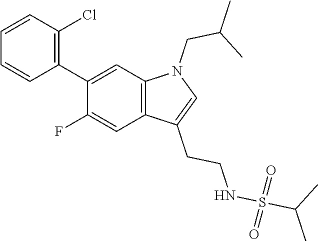 [SMILES: CC(C)Cn1cc(CCNS(=O)(=O)C(C)C)c2cc(F)c(-c3ccccc3Cl)cc21] | A | C |
| 263 | 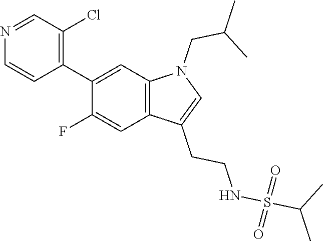 [SMILES: CC(C)Cn1cc(CCNS(=O)(=O)C(C)C)c2cc(F)c(-c3ccncc3Cl)cc21] | A | C |
| 264 | 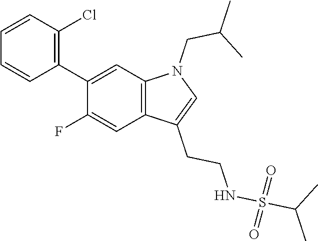 [SMILES: CC(C)Cn1cc(CCNS(=O)(=O)C(C)C)c2cc(F)c(-c3ccccc3Cl)cc21] | A | D |
| 265 | 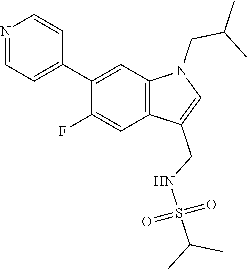 [SMILES: CC(C)Cn1cc(CNS(=O)(=O)C(C)C)c2cc(F)c(-c3ccncc3)cc21] | A | C |
| 266 | 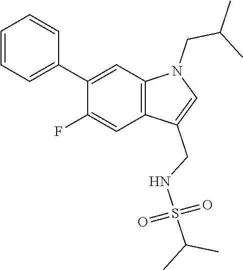 [SMILES: CC(C)Cn1cc(CNS(=O)(=O)C(C)C)c2cc(F)c(-c3ccccc3)cc21] | A | D |
| 267 | 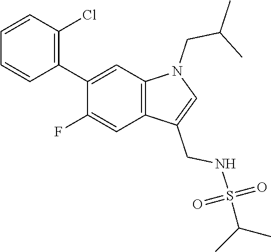 [SMILES: CC(C)Cn1cc(CNS(=O)(=O)C(C)C)c2cc(F)c(-c3ccccc3Cl)cc21] | A | D |
| 268 | 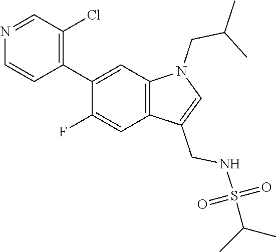 [SMILES: CC(C)Cn1cc(CNS(=O)(=O)C(C)C)c2cc(F)c(-c3ccncc3Cl)cc21] | A | D |
| 269 | 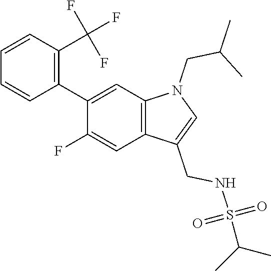 [SMILES: CC(C)Cn1cc(CNS(=O)(=O)C(C)C)c2cc(F)c(-c3ccccc3C(F)(F)F)cc21] | A | D |
| 270 | 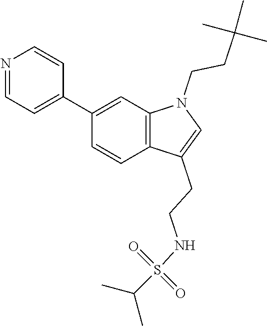 [SMILES: CC(C)S(=O)(=O)NCCc1cn(CCC(C)(C)C)c2cc(-c3ccncc3)ccc12] | A | D |
| 271 | 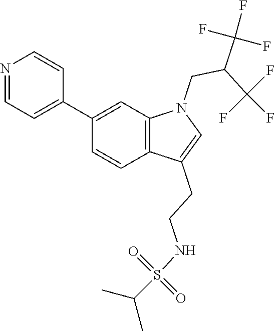 [SMILES: CC(C)S(=O)(=O)NCCc1cn(CC(C(F)(F)F)C(F)(F)F)c2cc(-c3ccncc3)ccc12] | B | D |
| 272 | 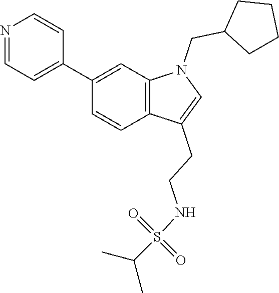 [SMILES: CC(C)S(=O)(=O)NCCc1cn(CC2CCCC2)c2cc(-c3ccncc3)ccc12] | A | D |
| 273 | 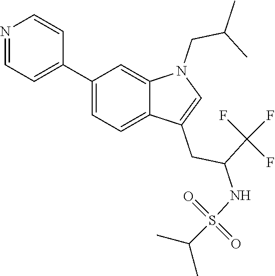 [SMILES: CC(C)Cn1cc(CC(NS(=O)(=O)C(C)C)C(F)(F)F)c2ccc(-c3ccncc3)cc21] | C | D |
| 274 | 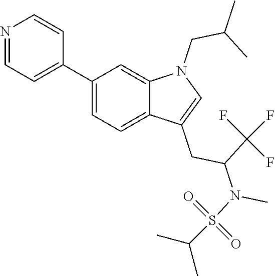 [SMILES: CC(C)Cn1cc(CC(N(C)S(=O)(=O)C(C)C)C(F)(F)F)c2ccc(-c3ccncc3)cc21] | C | D |
| 275 | 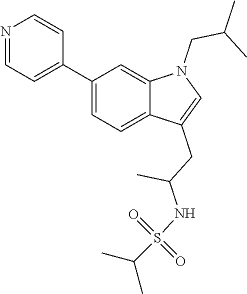 [SMILES: CC(C)Cn1cc(CC(C)NS(=O)(=O)C(C)C)c2ccc(-c3ccncc3)cc21] | B | D |
| 276 | 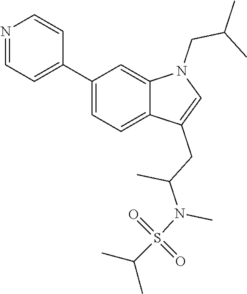 [SMILES: CC(C)Cn1cc(CC(C)N(C)S(=O)(=O)C(C)C)c2ccc(-c3ccncc3)cc21] | A | D |
| 277 | 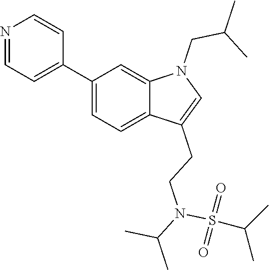 [SMILES: CC(C)Cn1cc(CCN(C(C)C)S(=O)(=O)C(C)C)c2ccc(-c3ccncc3)cc21] | A | D |
| 278 | 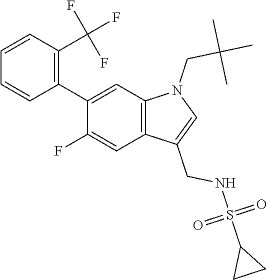 [SMILES: CC(C)(C)Cn1cc(CNS(=O)(=O)C2CC2)c2cc(F)c(-c3ccccc3C(F)(F)F)cc21] | A | B |
| 279 | 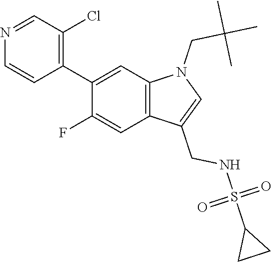 [SMILES: CC(C)(C)Cn1cc(CNS(=O)(=O)C2CC2)c2cc(F)c(-c3ccncc3Cl)cc21] | A | C |
| 280 | 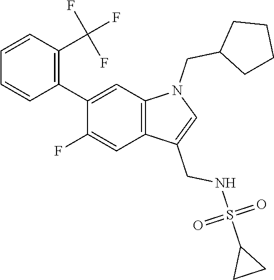 [SMILES: O=S(=O)(NCc1cn(CC2CCCC2)c2cc(-c3ccccc3C(F)(F)F)c(F)cc12)C1CC1] | A | D |
| 281 | 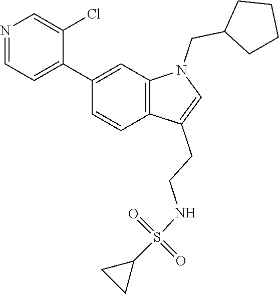 [SMILES: O=S(=O)(NCCc1cn(CC2CCCC2)c2cc(-c3ccncc3Cl)ccc12)C1CC1] | A | D |
| 282 | 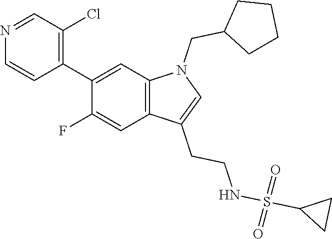 [SMILES: O=S(=O)(NCCc1cn(CC2CCCC2)c2cc(-c3ccncc3Cl)c(F)cc12)C1CC1] | A | D |
| 283 | 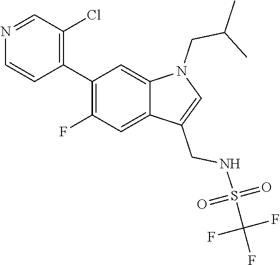 [SMILES: CC(C)Cn1cc(CNS(=O)(=O)C(F)(F)F)c2cc(F)c(-c3ccncc3Cl)cc21] | B | D |
| 284 | 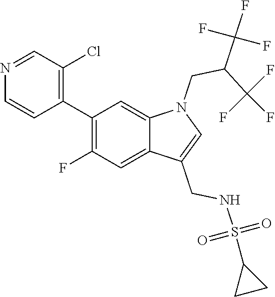 [SMILES: O=S(=O)(NCc1cn(CC(C(F)(F)F)C(F)(F)F)c2cc(-c3ccncc3Cl)c(F)cc12)C1CC1] | B | D |
| 285 | 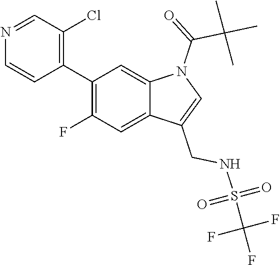 [SMILES: CC(C)(C)C(=O)n1cc(CNS(=O)(=O)C(F)(F)F)c2cc(F)c(-c3ccncc3Cl)cc21] | C | D |
| 286 | 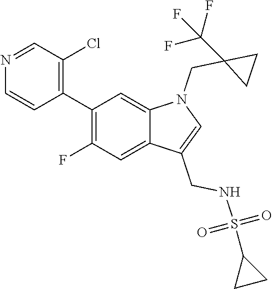 [SMILES: O=S(=O)(NCc1cn(CC2(C(F)(F)F)CC2)c2cc(-c3ccncc3Cl)c(F)cc12)C1CC1] | A | C |
| 287 | 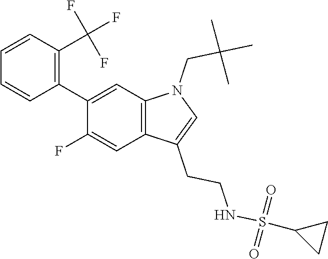 [SMILES: CC(C)(C)Cn1cc(CCNS(=O)(=O)C2CC2)c2cc(F)c(-c3ccccc3C(F)(F)F)cc21] | A | D |
| 288 | 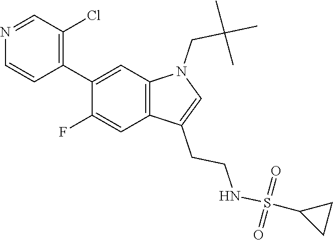 [SMILES: CC(C)(C)Cn1cc(CCNS(=O)(=O)C2CC2)c2cc(F)c(-c3ccncc3Cl)cc21] | A | D |
| 289 | 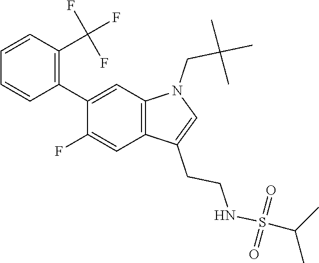 [SMILES: CC(C)S(=O)(=O)NCCc1cn(CC(C)(C)C)c2cc(-c3ccccc3C(F)(F)F)c(F)cc12] | A | D |
| 290 | 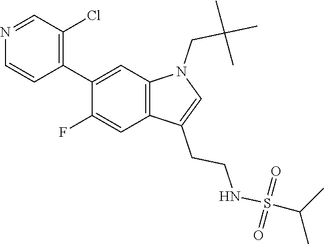 [SMILES: CC(C)S(=O)(=O)NCCc1cn(CC(C)(C)C)c2cc(-c3ccncc3Cl)c(F)cc12] | A | D |
| 291 | 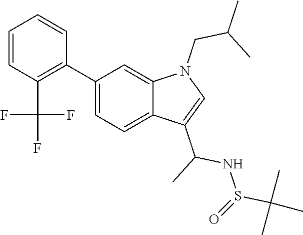 [SMILES: CC(C)Cn1cc(C(C)NS(=O)C(C)(C)C)c2ccc(-c3ccccc3C(F)(F)F)cc21] | C | D |
| 292 | 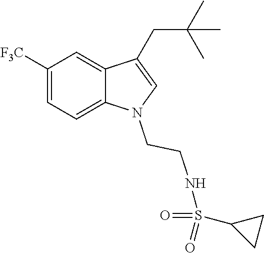 [SMILES: CC(C)(C)Cc1cn(CCNS(=O)(=O)C2CC2)c2ccc(C(F)(F)F)cc12] | B | D |
| 293 | 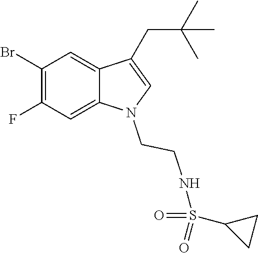 [SMILES: CC(C)(C)Cc1cn(CCNS(=O)(=O)C2CC2)c2cc(F)c(Br)cc12] | C | D |
| 294 | 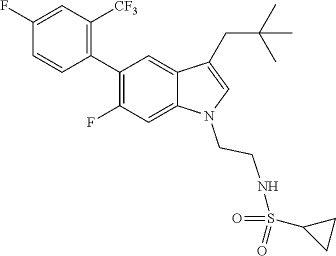 [SMILES: CC(C)(C)Cc1cn(CCNS(=O)(=O)C2CC2)c2cc(F)c(-c3ccc(F)cc3C(F)(F)F)cc12] | A | D |
| 295 | 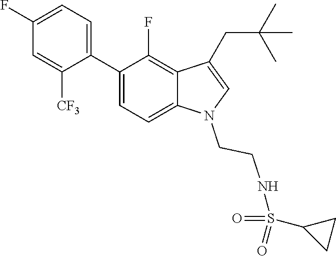 [SMILES: CC(C)(C)Cc1cn(CCNS(=O)(=O)C2CC2)c2ccc(-c3ccc(F)cc3C(F)(F)F)c(F)c12] | B | D |
| 296 | 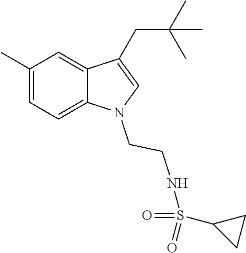 [SMILES: Cc1ccc2c(c1)c(CC(C)(C)C)cn2CCNS(=O)(=O)C1CC1] | B | C |
| 297 | 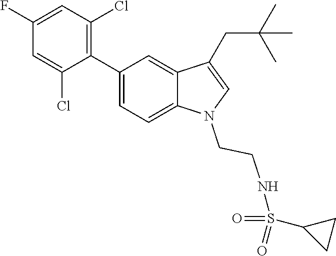 [SMILES: CC(C)(C)Cc1cn(CCNS(=O)(=O)C2CC2)c2ccc(-c3c(Cl)cc(F)cc3Cl)cc12] | B | B |
| 298 | 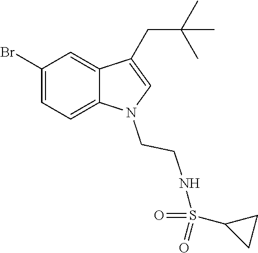 [SMILES: CC(C)(C)Cc1cn(CCNS(=O)(=O)C2CC2)c2ccc(Br)cc12] | B | C |
| 299 | 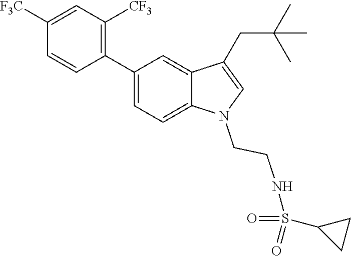 [SMILES: CC(C)(C)Cc1cn(CCNS(=O)(=O)C2CC2)c2ccc(-c3ccc(C(F)(F)F)cc3C(F)(F)F)cc12] | A | B |
| 300 | 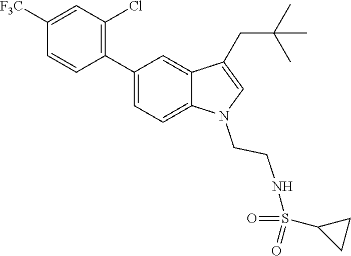 [SMILES: CC(C)(C)Cc1cn(CCNS(=O)(=O)C2CC2)c2ccc(-c3ccc(C(F)(F)F)cc3Cl)cc12] | A | B |
| 301 | 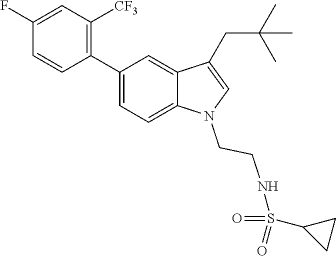 [SMILES: CC(C)(C)Cc1cn(CCNS(=O)(=O)C2CC2)c2ccc(-c3ccc(F)cc3C(F)(F)F)cc12] | A | B |
| 302 | 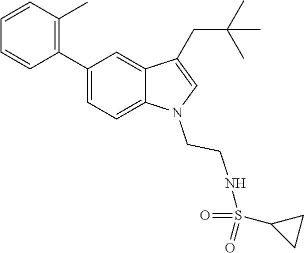 [SMILES: Cc1ccccc1-c1ccc2c(c1)c(CC(C)(C)C)cn2CCNS(=O)(=O)C1CC1] | A | D |
| 303 | 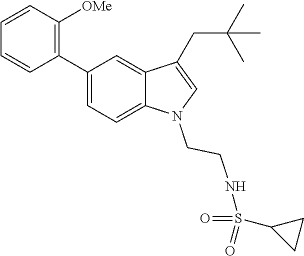 [SMILES: COc1ccccc1-c1ccc2c(c1)c(CC(C)(C)C)cn2CCNS(=O)(=O)C1CC1] | A | D |
| 304 | 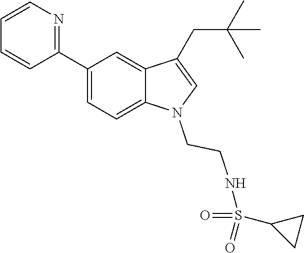 [SMILES: CC(C)(C)Cc1cn(CCNS(=O)(=O)C2CC2)c2ccc(-c3ccccn3)cc12] | B | D |
| 305 | 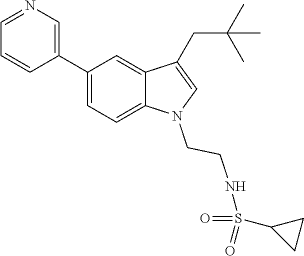 [SMILES: CC(C)(C)Cc1cn(CCNS(=O)(=O)C2CC2)c2ccc(-c3cccnc3)cc12] | B | D |
| 306 | 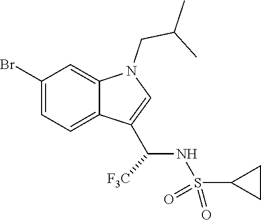 [SMILES: CC(C)Cn1cc([C@H](NS(=O)(=O)C2CC2)C(F)(F)F)c2ccc(Br)cc21] | A | D |
| 307 | 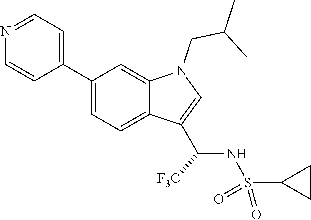 [SMILES: CC(C)Cn1cc([C@H](NS(=O)(=O)C2CC2)C(F)(F)F)c2ccc(-c3ccncc3)cc21] | A | D |
| 308 | 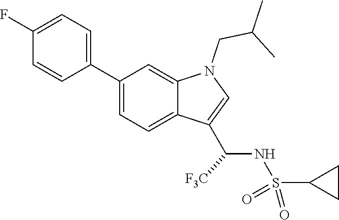 [SMILES: CC(C)Cn1cc([C@H](NS(=O)(=O)C2CC2)C(F)(F)F)c2ccc(-c3ccc(F)cc3)cc21] | B | D |
| 309 | 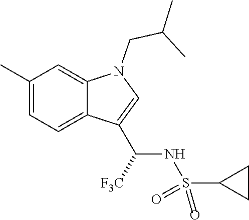 | A | A |
| 310 | 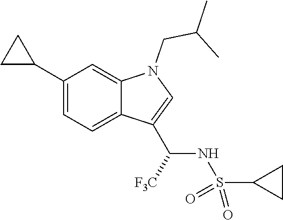 [SMILES: CC(C)Cn1cc([C@H](NS(=O)(=O)C2CC2)C(F)(F)F)c2ccc(C3CC3)cc21] | A | B |
| 311 | 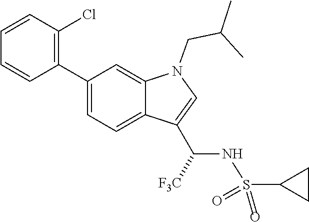 | A | D |
| 312 | 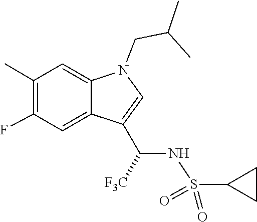 | A | D |
| 313 | 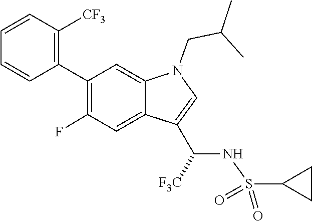 | A | C |
| 314 | 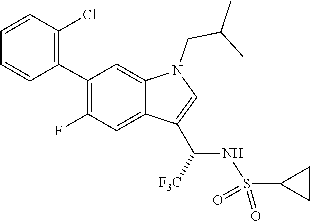 | A | D |
| 315 | 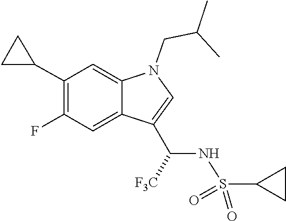 | A | D |
| 316 | 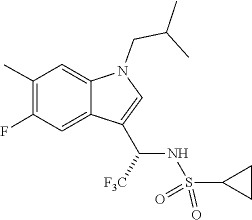 [SMILES: Cc1cc2c(cc1F)c([C@H](NS(=O)(=O)C1CC1)C(F)(F)F)cn2CC(C)C] | A | D |
| 317 | 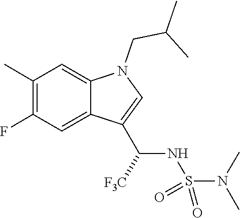 [SMILES: Cc1cc2c(cc1F)c([C@H](NS(=O)(=O)N(C)C)C(F)(F)F)cn2CC(C)C] | A | D |
| 318 | 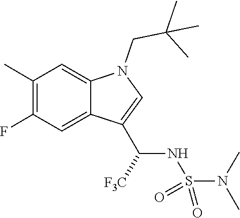 [SMILES: Cc1cc2c(cc1F)c([C@H](NS(=O)(=O)N(C)C)C(F)(F)F)cn2CC(C)(C)C] | A | D |
| 319 | 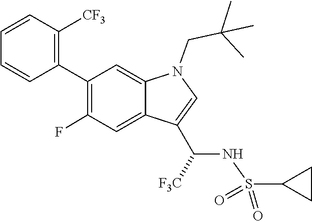 | A | D |
| 320 | 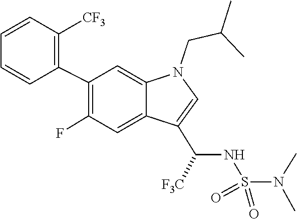 | A | D |
| 321 | 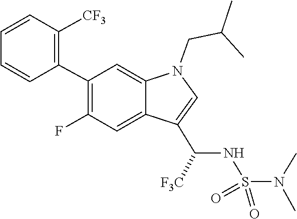 [SMILES: CC(C)Cn1cc([C@H](NS(=O)(=O)N(C)C)C(F)(F)F)c2cc(F)c(-c3ccccc3C(F)(F)F)cc21] | A | D |
| 322 | 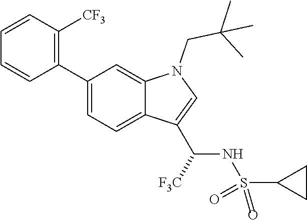 | A | B |
| 323 | 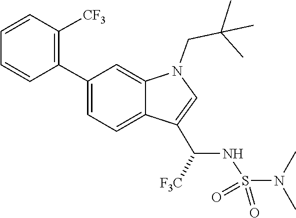 [SMILES: CN(C)S(=O)(=O)N[C@@H](c1cn(CC(C)(C)C)c2cc(-c3ccccc3C(F)(F)F)ccc12)C(F)(F)F] | A | C |
| 324 | 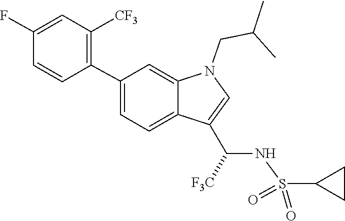 [SMILES: CC(C)Cn1cc([C@H](NS(=O)(=O)C2CC2)C(F)(F)F)c2ccc(-c3ccc(F)cc3C(F)(F)F)cc21] | A | D |
| 325 | 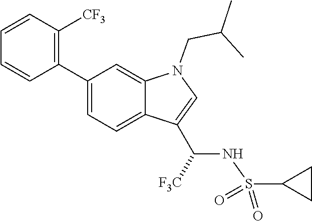 | A | D |
| 326 | 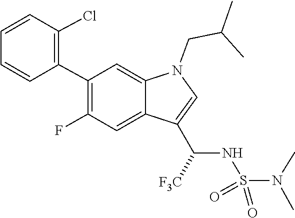 | A | D |
| 327 | 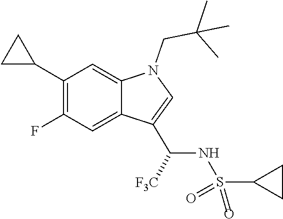 | A | D |
| 328 | 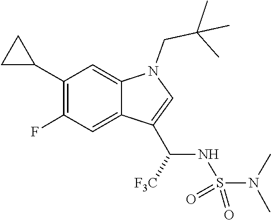 [SMILES: CN(C)S(=O)(=O)N[C@@H](c1cn(CC(C)(C)C)c2cc(C3CC3)c(F)cc12)C(F)(F)F] | A | C |
| 329 | 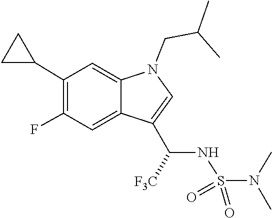 | A | C |
| 330 | 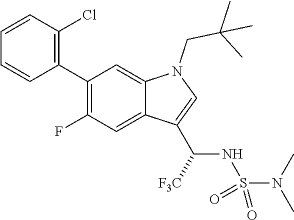 [SMILES: CN(C)S(=O)(=O)N[C@@H](c1cn(CC(C)(C)C)c2cc(-c3ccccc3Cl)c(F)cc12)C(F)(F)F] | A | D |
| 331 | 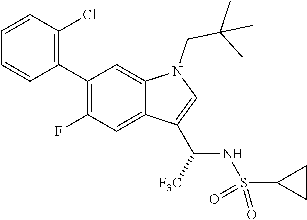 [SMILES: CC(C)(C)Cn1cc([C@H](NS(=O)(=O)C2CC2)C(F)(F)F)c2cc(F)c(-c3ccccc3Cl)cc21] | A | B |
| 332 | 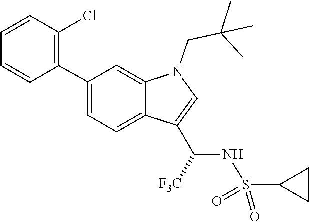 | A | D |
| 333 | 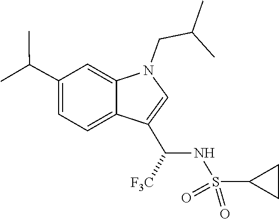 [SMILES: CC(C)Cn1cc([C@H](NS(=O)(=O)C2CC2)C(F)(F)F)c2ccc(C(C)C)cc21] | A | C |
| 334 | 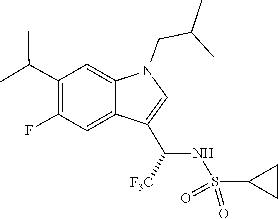 [SMILES: CC(C)Cn1cc([C@H](NS(=O)(=O)C2CC2)C(F)(F)F)c2cc(F)c(C(C)C)cc21] | B | D |
| 335 | 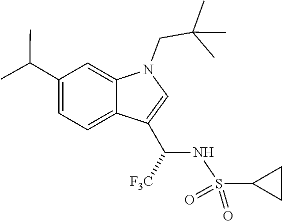 | A | D |
| 336 | 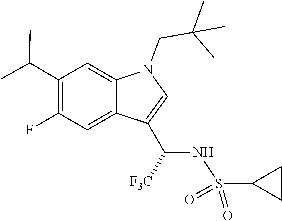 | B | D |
| 337 | 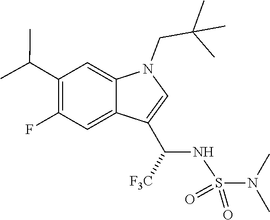 | B | D |
| 338 | 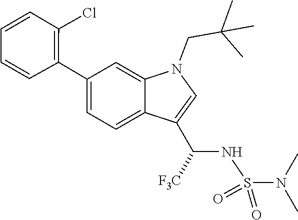 | A | D |
| 339 | 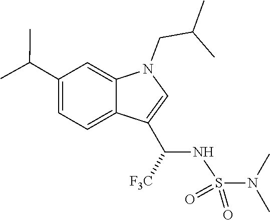 | A | D |
| 340 | 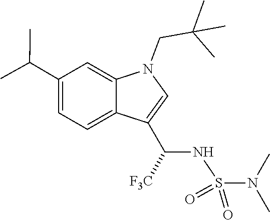 | A | B |
| 341 | 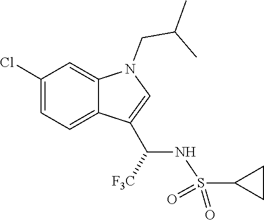 | A | D |
| 342 | 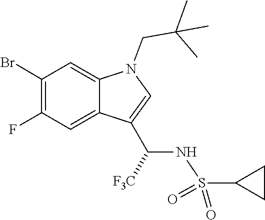 | A | C |
| 343 | 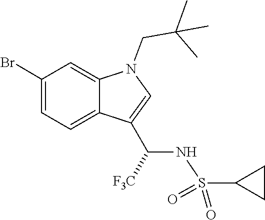 | A | D |
| 344 | 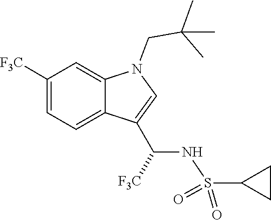 | A | C |
| 346 | 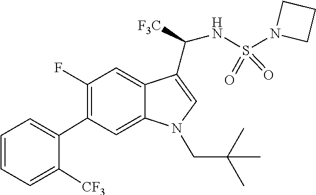 [SMILES: CC(C)(C)Cn1cc([C@H](NS(=O)(=O)N2CCC2)C(F)(F)F)c2cc(F)c(-c3ccccc3C(F)(F)F)cc21] | A | D |
| 347 | 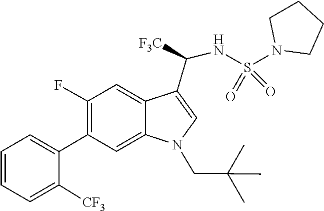 [SMILES: CC(C)(C)Cn1cc([C@H](NS(=O)(=O)N2CCCC2)C(F)(F)F)c2cc(F)c(-c3ccccc3C(F)(F)F)cc21] | A | D |
| 348 | 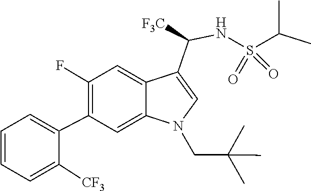 [SMILES: CC(C)S(=O)(=O)N[C@@H](c1cn(CC(C)(C)C)c2cc(-c3ccccc3C(F)(F)F)c(F)cc12)C(F)(F)F] | B | D |
| 349 | 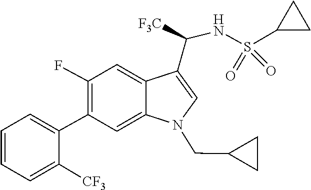 [SMILES: O=S(=O)(N[C@@H](c1cn(CC2CC2)c2cc(-c3ccccc3C(F)(F)F)c(F)cc12)C(F)(F)F)C1CC1] | A | D |
| 350 | 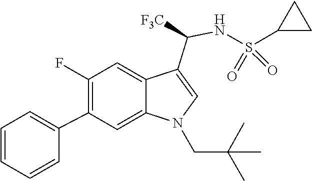 [SMILES: CC(C)(C)Cn1cc([C@H](NS(=O)(=O)C2CC2)C(F)(F)F)c2cc(F)c(-c3ccccc3)cc21] | B | D |
| 351 | 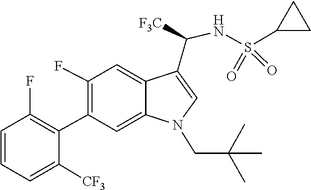 [SMILES: CC(C)(C)Cn1cc([C@H](NS(=O)(=O)C2CC2)C(F)(F)F)c2cc(F)c(-c3c(F)cccc3C(F)(F)F)cc21] | A | D |
| 353 | 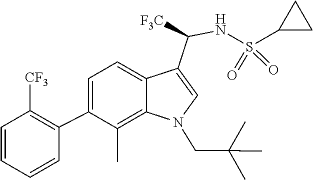 [SMILES: Cc1c(-c2ccccc2C(F)(F)F)ccc2c([C@H](NS(=O)(=O)C3CC3)C(F)(F)F)cn(CC(C)(C)C)c12] | A | D |
| 355 | 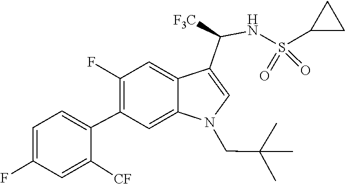 | A | D |
| 357 | 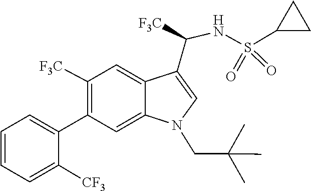 [SMILES: CC(C)(C)Cn1cc([C@H](NS(=O)(=O)C2CC2)C(F)(F)F)c2cc(C(F)(F)F)c(-c3ccccc3C(F)(F)F)cc21] | B | D |
| 360 | 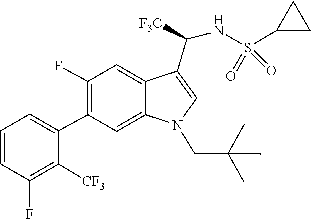 [SMILES: CC(C)(C)Cn1cc([C@H](NS(=O)(=O)C2CC2)C(F)(F)F)c2cc(F)c(-c3cccc(F)c3C(F)(F)F)cc21] | A | D |
| 361 | 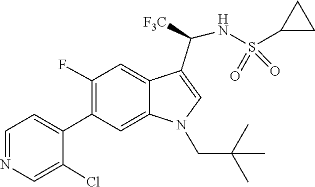 [SMILES: CC(C)(C)Cn1cc([C@H](NS(=O)(=O)C2CC2)C(F)(F)F)c2cc(F)c(-c3ccncc3Cl)cc21] | A | D |
| 365 | 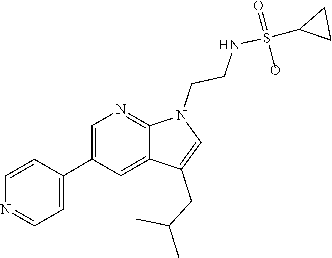 | A | C |
| 366 | 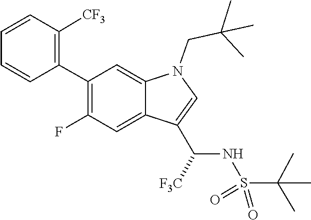 | B | D |
| 367 | 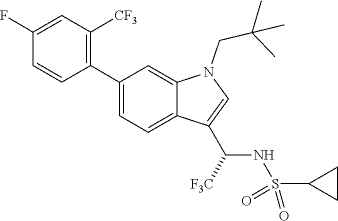 [SMILES: CC(C)(C)Cn1cc([C@H](NS(=O)(=O)C2CC2)C(F)(F)F)c2ccc(-c3ccc(F)cc3C(F)(F)F)cc21] | A | D |
| 368 | 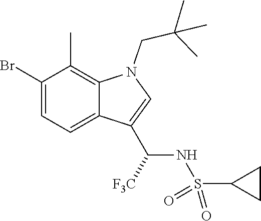 [SMILES: Cc1c(Br)ccc2c([C@H](NS(=O)(=O)C3CC3)C(F)(F)F)cn(CC(C)(C)C)c12] | B | D |
| 369 | 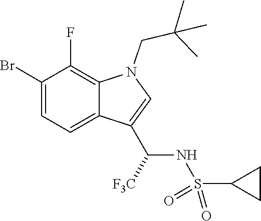 [SMILES: CC(C)(C)Cn1cc([C@H](NS(=O)(=O)C2CC2)C(F)(F)F)c2ccc(Br)c(F)c21] | B | D |
| 370 | 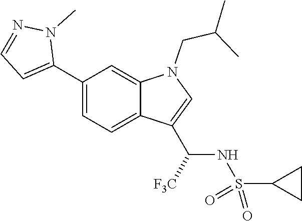 [SMILES: CC(C)Cn1cc([C@H](NS(=O)(=O)C2CC2)C(F)(F)F)c2ccc(-c3ccnn3C)cc21] | A | D |
| 371 | 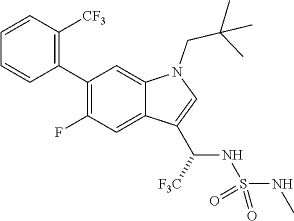 [SMILES: CNS(=O)(=O)N[C@@H](c1cn(CC(C)(C)C)c2cc(-c3ccccc3C(F)(F)F)c(F)cc12)C(F)(F)F] | B | D |
| 372 | 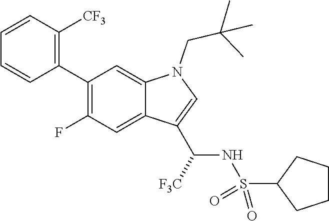 [SMILES: CC(C)(C)Cn1cc([C@H](NS(=O)(=O)C2CCCC2)C(F)(F)F)c2cc(F)c(-c3ccccc3C(F)(F)F)cc21] | B | D |
| 373 | 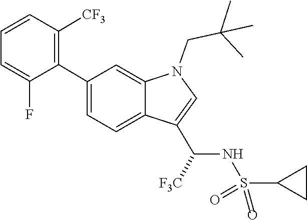 | A | D |
| 374 | 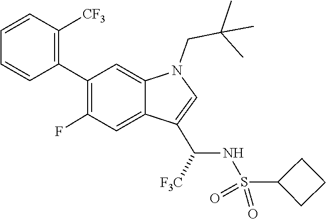 [SMILES: CC(C)(C)Cn1cc([C@H](NS(=O)(=O)C2CCC2)C(F)(F)F)c2cc(F)c(-c3ccccc3C(F)(F)F)cc21] | A | D |
| 375 | 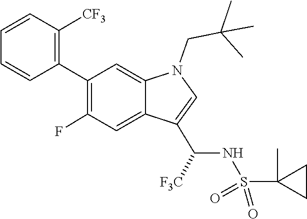 [SMILES: CC(C)(C)Cn1cc([C@H](NS(=O)(=O)C2(C)CC2)C(F)(F)F)c2cc(F)c(-c3ccccc3C(F)(F)F)cc21] | A | D |
| 376 | 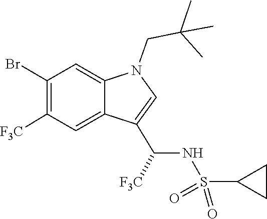 [SMILES: CC(C)(C)Cn1cc([C@H](NS(=O)(=O)C2CC2)C(F)(F)F)c2cc(C(F)(F)F)c(Br)cc21] | C | D |
| 377 | 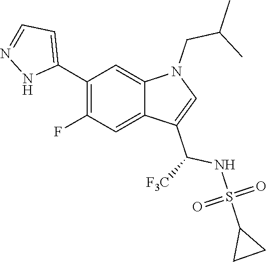 [SMILES: CC(C)Cn1cc([C@H](NS(=O)(=O)C2CC2)C(F)(F)F)c2cc(F)c(-c3ccn[nH]3)cc21] | B | D |
| 378 | 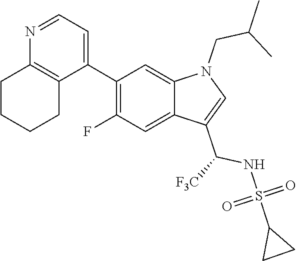 [SMILES: CC(C)Cn1cc([C@H](NS(=O)(=O)C2CC2)C(F)(F)F)c2cc(F)c(-c3ccnc4c3CCCC4)cc21] | A | D |
| 379 | 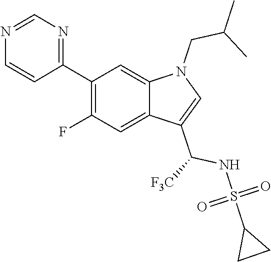 [SMILES: CC(C)Cn1cc([C@H](NS(=O)(=O)C2CC2)C(F)(F)F)c2cc(F)c(-c3ccncn3)cc21] | A | D |
| 380 | 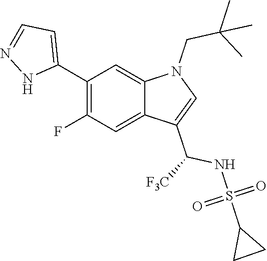 [SMILES: CC(C)(C)Cn1cc([C@H](NS(=O)(=O)C2CC2)C(F)(F)F)c2cc(F)c(-c3ccn[nH]3)cc21] | A | D |
| 381 | 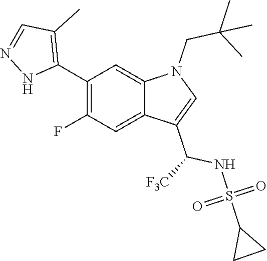 [SMILES: Cc1cn[nH]c1-c1cc2c(cc1F)c([C@H](NS(=O)(=O)C1CC1)C(F)(F)F)cn2CC(C)(C)C] | B | D |
| 382 | 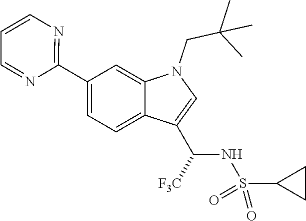 [SMILES: CC(C)(C)Cn1cc([C@H](NS(=O)(=O)C2CC2)C(F)(F)F)c2ccc(-c3ncccn3)cc21] | B | D |
| 383 | 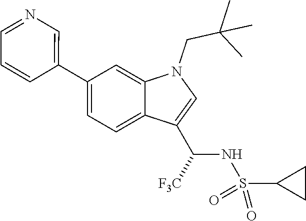 [SMILES: CC(C)(C)Cn1cc([C@H](NS(=O)(=O)C2CC2)C(F)(F)F)c2ccc(-c3cccnc3)cc21] | B | D |
| 384 | 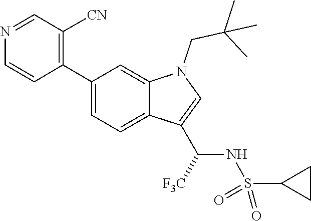 [SMILES: CC(C)(C)Cn1cc([C@H](NS(=O)(=O)C2CC2)C(F)(F)F)c2ccc(-c3ccncc3C#N)cc21] | A | D |
| 385 | 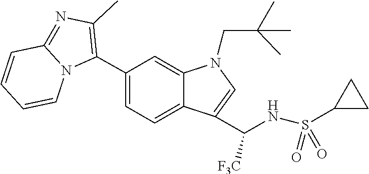 [SMILES: Cc1nc2ccccn2c1-c1ccc2c([C@H](NS(=O)(=O)C3CC3)C(F)(F)F)cn(CC(C)(C)C)c2c1] | B | D |
| 386 | 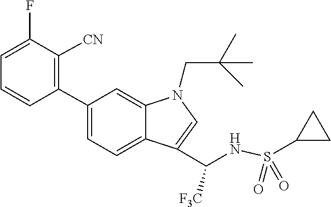 [SMILES: CC(C)(C)Cn1cc([C@H](NS(=O)(=O)C2CC2)C(F)(F)F)c2ccc(-c3cccc(F)c3C#N)cc21] | B | D |
| 387 | 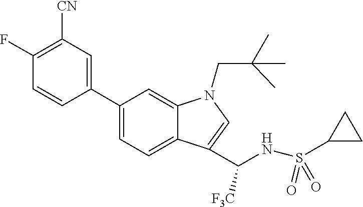 [SMILES: CC(C)(C)Cn1cc([C@H](NS(=O)(=O)C2CC2)C(F)(F)F)c2ccc(-c3ccc(F)c(C#N)c3)cc21] | B | D |
| 388 | 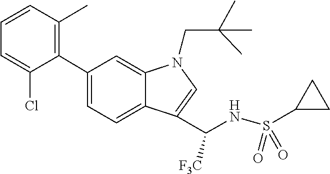 [SMILES: Cc1cccc(Cl)c1-c1ccc2c([C@H](NS(=O)(=O)C3CC3)C(F)(F)F)cn(CC(C)(C)C)c2c1] | A | D |
| 389 | 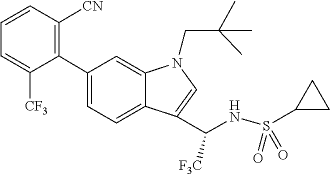 [SMILES: CC(C)(C)Cn1cc([C@H](NS(=O)(=O)C2CC2)C(F)(F)F)c2ccc(-c3c(C#N)cccc3C(F)(F)F)cc21] | A | D |
| 390 | 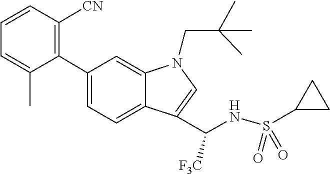 [SMILES: Cc1cccc(C#N)c1-c1ccc2c([C@H](NS(=O)(=O)C3CC3)C(F)(F)F)cn(CC(C)(C)C)c2c1] | A | D |
| 391 | 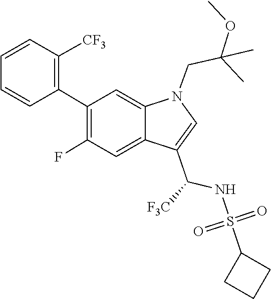 [SMILES: COC(C)(C)Cn1cc([C@H](NS(=O)(=O)C2CCC2)C(F)(F)F)c2cc(F)c(-c3ccccc3C(F)(F)F)cc21] | A | D |
| 392 | 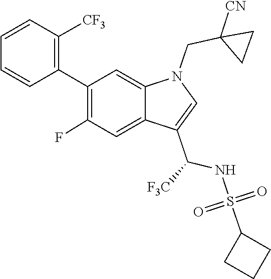 [SMILES: N#CC1(Cn2cc([C@H](NS(=O)(=O)C3CCC3)C(F)(F)F)c3cc(F)c(-c4ccccc4C(F)(F)F)cc32)CC1] | A | D |
| 393 | 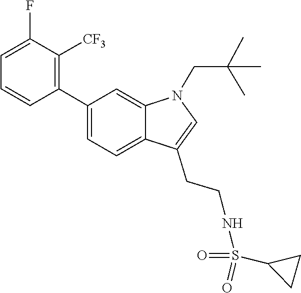 [SMILES: CC(C)(C)Cn1cc(CCNS(=O)(=O)C2CC2)c2ccc(-c3cccc(F)c3C(F)(F)F)cc21] | B | D |
| 394 | 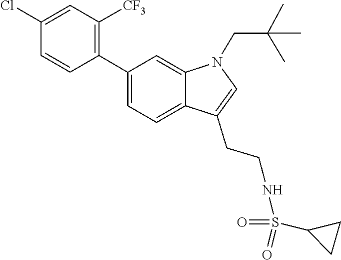 [SMILES: CC(C)(C)Cn1cc(CCNS(=O)(=O)C2CC2)c2ccc(-c3ccc(Cl)cc3C(F)(F)F)cc21] | A | D |
| 395 | 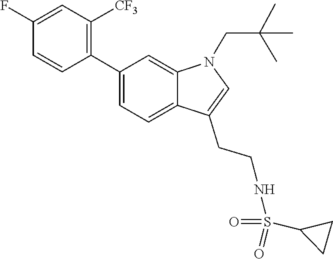 [SMILES: CC(C)(C)Cn1cc(CCNS(=O)(=O)C2CC2)c2ccc(-c3ccc(F)cc3C(F)(F)F)cc21] | A | D |
| 396 | 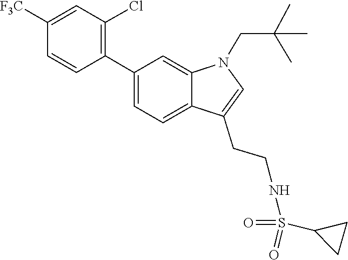 [SMILES: CC(C)(C)Cn1cc(CCNS(=O)(=O)C2CC2)c2ccc(-c3ccc(C(F)(F)F)cc3Cl)cc21] | A | D |
| 397 | 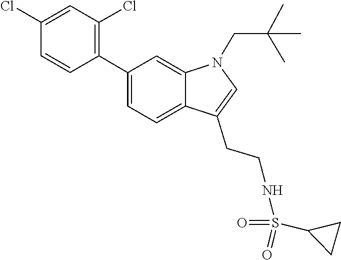 [SMILES: CC(C)(C)Cn1cc(CCNS(=O)(=O)C2CC2)c2ccc(-c3ccc(Cl)cc3Cl)cc21] | A | D |
| 398 | 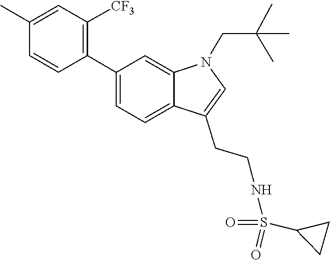 [SMILES: Cc1ccc(-c2ccc3c(CCNS(=O)(=O)C4CC4)cn(CC(C)(C)C)c3c2)c(C(F)(F)F)c1] | A | D |
| 399 | 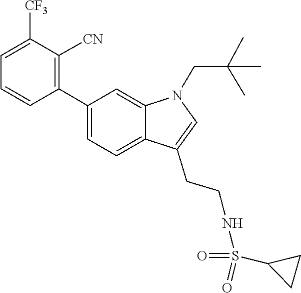 [SMILES: CC(C)(C)Cn1cc(CCNS(=O)(=O)C2CC2)c2ccc(-c3cccc(C(F)(F)F)c3C#N)cc21] | C | D |
| 400 | 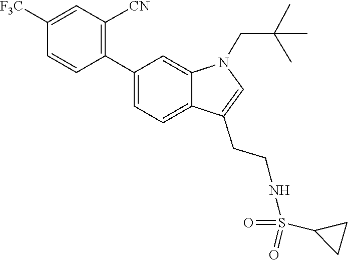 [SMILES: CC(C)(C)Cn1cc(CCNS(=O)(=O)C2CC2)c2ccc(-c3ccc(C(F)(F)F)cc3C#N)cc21] | A | D |
| 401 | 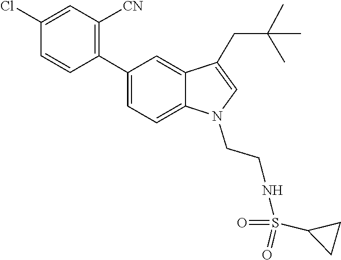 [SMILES: CC(C)(C)Cc1cn(CCNS(=O)(=O)C2CC2)c2ccc(-c3ccc(Cl)cc3C#N)cc12] | A | D |
| 402 | 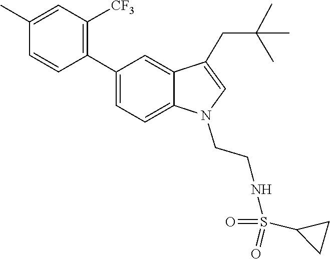 [SMILES: Cc1ccc(-c2ccc3c(c2)c(CC(C)(C)C)cn3CCNS(=O)(=O)C2CC2)c(C(F)(F)F)c1] | A | D |
| 403 | 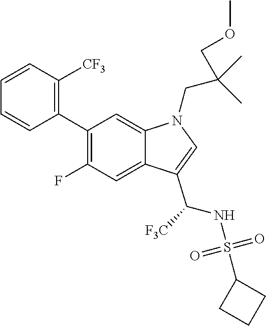 [SMILES: COCC(C)(C)Cn1cc([C@H](NS(=O)(=O)C2CCC2)C(F)(F)F)c2cc(F)c(-c3ccccc3C(F)(F)F)cc21] | A | D |
| 404 | 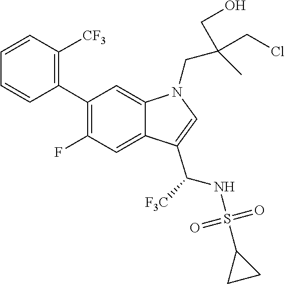 [SMILES: CC(CO)(CCl)Cn1cc([C@H](NS(=O)(=O)C2CC2)C(F)(F)F)c2cc(F)c(-c3ccccc3C(F)(F)F)cc21] | B | D |
| 405 | 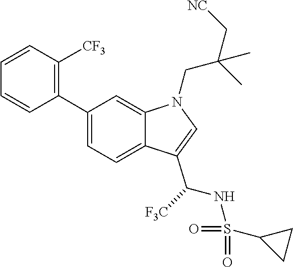 [SMILES: CC(C)(CC#N)Cn1cc([C@H](NS(=O)(=O)C2CC2)C(F)(F)F)c2ccc(-c3ccccc3C(F)(F)F)cc21] | A | D |
| 406 | 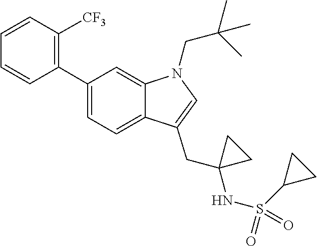 [SMILES: CC(C)(C)Cn1cc(CC2(NS(=O)(=O)C3CC3)CC2)c2ccc(-c3ccccc3C(F)(F)F)cc21] | C | D |
| 407 | 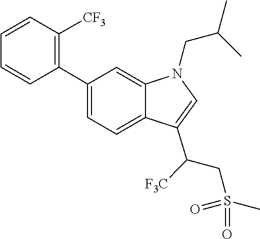 [SMILES: CC(C)Cn1cc(C(CS(C)(=O)=O)C(F)(F)F)c2ccc(-c3ccccc3C(F)(F)F)cc21] | C | D |
| 408 | 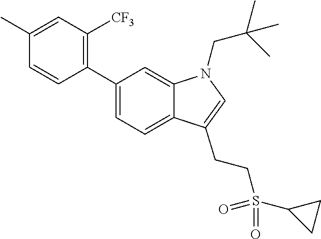 [SMILES: Cc1ccc(-c2ccc3c(CCS(=O)(=O)C4CC4)cn(CC(C)(C)C)c3c2)c(C(F)(F)F)c1] | A | D |
| 409 | 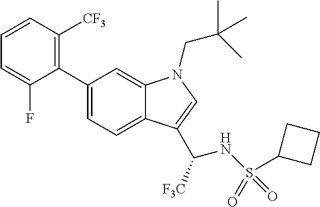 [SMILES: CC(C)(C)Cn1cc([C@H](NS(=O)(=O)C2CCC2)C(F)(F)F)c2ccc(-c3c(F)cccc3C(F)(F)F)cc21] | A | D |
| 410 | 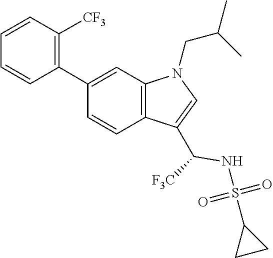 [SMILES: CC(C)Cn1cc([C@H](NS(=O)(=O)C2CC2)C(F)(F)F)c2ccc(-c3ccccc3C(F)(F)F)cc21] | A | D |
| 411 | 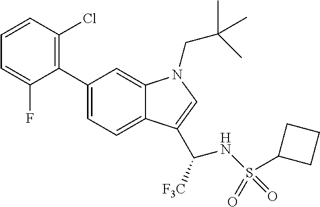 [SMILES: CC(C)(C)Cn1cc([C@H](NS(=O)(=O)C2CCC2)C(F)(F)F)c2ccc(-c3c(F)cccc3Cl)cc21] | A | D |
| 412 | 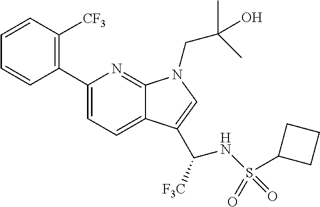 [SMILES: CC(C)(O)Cn1cc([C@H](NS(=O)(=O)C2CCC2)C(F)(F)F)c2ccc(-c3ccccc3C(F)(F)F)nc21] | B | D |
| 413 | 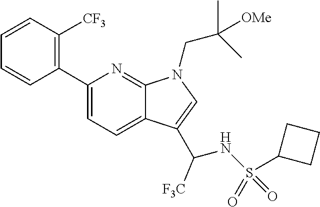 [SMILES: COC(C)(C)Cn1cc(C(NS(=O)(=O)C2CCC2)C(F)(F)F)c2ccc(-c3ccccc3C(F)(F)F)nc21] | A | D |
| 414 | 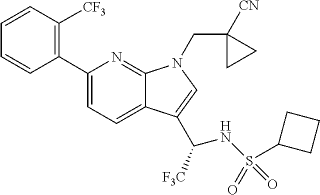 [SMILES: N#CC1(Cn2cc([C@H](NS(=O)(=O)C3CCC3)C(F)(F)F)c3ccc(-c4ccccc4C(F)(F)F)nc32)CC1] | A | D |
| 415 | 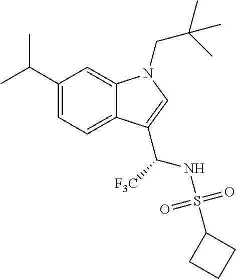 [SMILES: CC(C)c1ccc2c([C@H](NS(=O)(=O)C3CCC3)C(F)(F)F)cn(CC(C)(C)C)c2c1] | B | D |
| 416 | 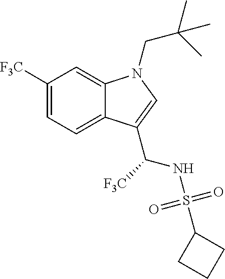 [SMILES: CC(C)(C)Cn1cc([C@H](NS(=O)(=O)C2CCC2)C(F)(F)F)c2ccc(C(F)(F)F)cc21] | C | D |
| 417 | 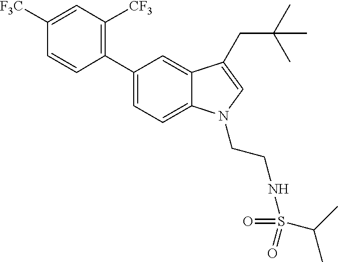 [SMILES: CC(C)S(=O)(=O)NCCn1cc(CC(C)(C)C)c2cc(-c3ccc(C(F)(F)F)cc3C(F)(F)F)ccc21] | A | D |
| 418 | 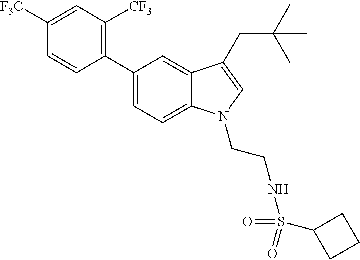 [SMILES: CC(C)(C)Cc1cn(CCNS(=O)(=O)C2CCC2)c2ccc(-c3ccc(C(F)(F)F)cc3C(F)(F)F)cc12] | A | D |
| 419 | 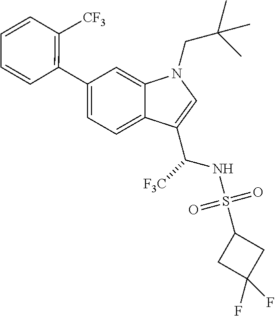 [SMILES: CC(C)(C)Cn1cc([C@H](NS(=O)(=O)C2CC(F)(F)C2)C(F)(F)F)c2ccc(-c3ccccc3C(F)(F)F)cc21] | B | D |
| 420 | 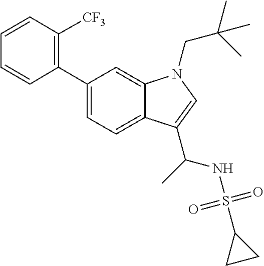 [SMILES: CC(NS(=O)(=O)C1CC1)c1cn(CC(C)(C)C)c2cc(-c3ccccc3C(F)(F)F)ccc12] | A | D |
| 421 | 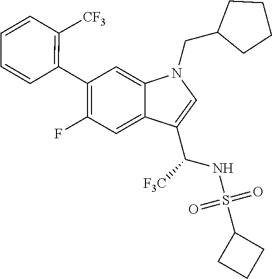 [SMILES: O=S(=O)(N[C@@H](c1cn(CC2CCCC2)c2cc(-c3ccccc3C(F)(F)F)c(F)cc12)C(F)(F)F)C1CCC1] | A | D |
| 422 | 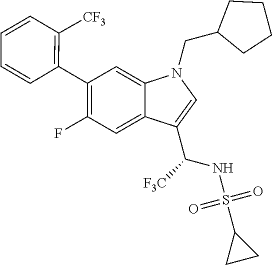 [SMILES: O=S(=O)(N[C@@H](c1cn(CC2CCCC2)c2cc(-c3ccccc3C(F)(F)F)c(F)cc12)C(F)(F)F)C1CC1] | A | D |
| 423 | 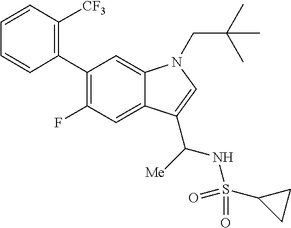 [SMILES: CC(NS(=O)(=O)C1CC1)c1cn(CC(C)(C)C)c2cc(-c3ccccc3C(F)(F)F)c(F)cc12] | A | D |
| 424 | 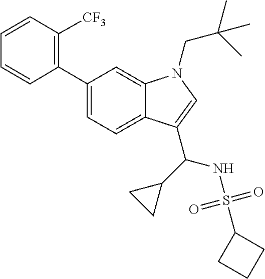 [SMILES: CC(C)(C)Cn1cc(C(NS(=O)(=O)C2CCC2)C2CC2)c2ccc(-c3ccccc3C(F)(F)F)cc21] | A | D |
| 425 | 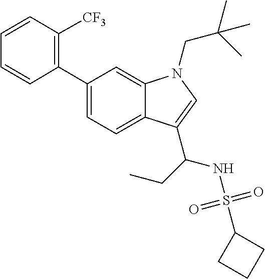 [SMILES: CCC(NS(=O)(=O)C1CCC1)c1cn(CC(C)(C)C)c2cc(-c3ccccc3C(F)(F)F)ccc12] | A | D |
| 426 | 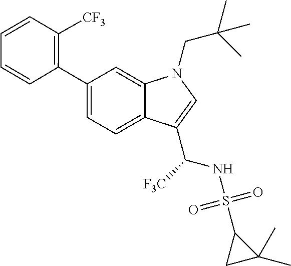 [SMILES: CC(C)(C)Cn1cc([C@H](NS(=O)(=O)C2CC2(C)C)C(F)(F)F)c2ccc(-c3ccccc3C(F)(F)F)cc21] | B | D |
| 427 | 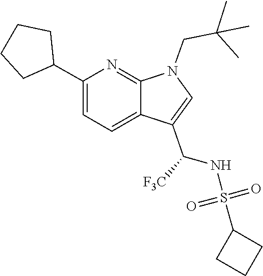 [SMILES: CC(C)(C)Cn1cc([C@H](NS(=O)(=O)C2CCC2)C(F)(F)F)c2ccc(C3CCCC3)nc21] | A | D |
| 428 | 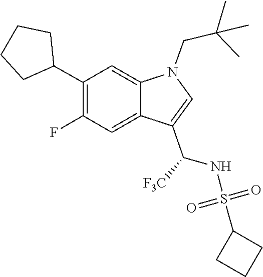 [SMILES: CC(C)(C)Cn1cc([C@H](NS(=O)(=O)C2CCC2)C(F)(F)F)c2cc(F)c(C3CCCC3)cc21] | A | D |
| 429 | 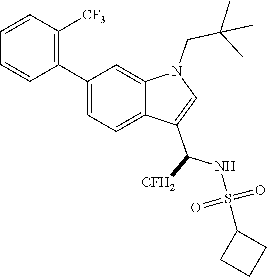 | A | D |
| 430 | 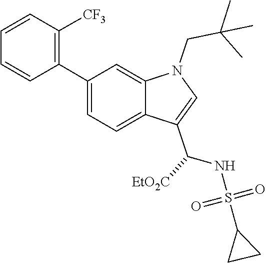 [SMILES: CCOC(=O)[C@@H](NS(=O)(=O)C1CC1)c1cn(CC(C)(C)C)c2cc(-c3ccccc3C(F)(F)F)ccc12] | C | D |
| 431 | 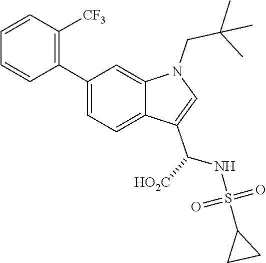 [SMILES: CC(C)(C)Cn1cc([C@H](NS(=O)(=O)C2CC2)C(=O)O)c2ccc(-c3ccccc3C(F)(F)F)cc21] | C | D |
| 432 | 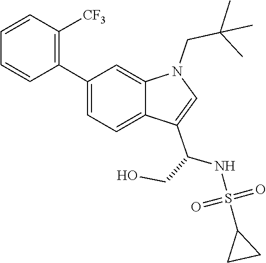 [SMILES: CC(C)(C)Cn1cc([C@@H](CO)NS(=O)(=O)C2CC2)c2ccc(-c3ccccc3C(F)(F)F)cc21] | A | D |
| 433 | 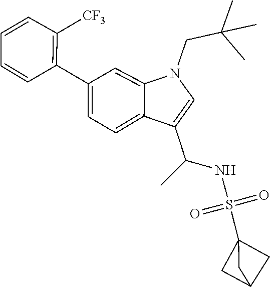 [SMILES: CC(NS(=O)(=O)C12CC(C1)C2)c1cn(CC(C)(C)C)c2cc(-c3ccccc3C(F)(F)F)ccc12] | A | D |
| 434 | 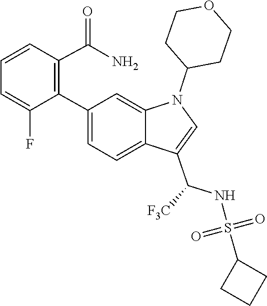 [SMILES: NC(=O)c1cccc(F)c1-c1ccc2c([C@H](NS(=O)(=O)C3CCC3)C(F)(F)F)cn(C3CCOCC3)c2c1] | C | D |
| 435 | 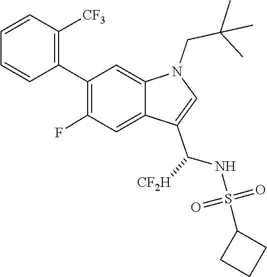 [SMILES: CC(C)(C)Cn1cc([C@H](NS(=O)(=O)C2CCC2)C(F)F)c2cc(F)c(-c3ccccc3C(F)(F)F)cc21] | A | D |
| 436 | 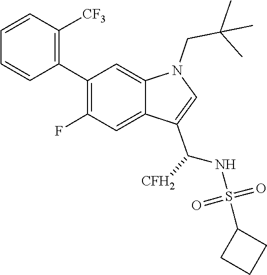 | A | D |
| 437 | 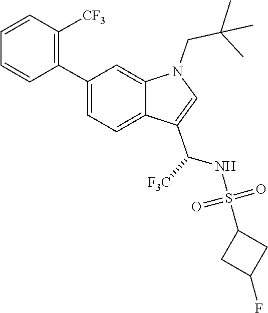 [SMILES: CC(C)(C)Cn1cc([C@H](NS(=O)(=O)C2CC(F)C2)C(F)(F)F)c2ccc(-c3ccccc3C(F)(F)F)cc21] | B | D |
| 438 | 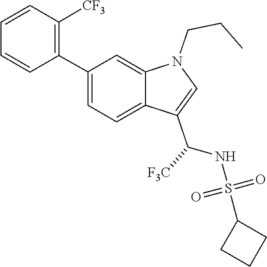 [SMILES: CCCn1cc([C@H](NS(=O)(=O)C2CCC2)C(F)(F)F)c2ccc(-c3ccccc3C(F)(F)F)cc21] | A | D |
| 439 | 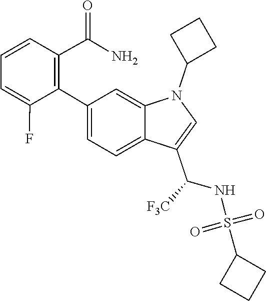 [SMILES: NC(=O)c1cccc(F)c1-c1ccc2c([C@H](NS(=O)(=O)C3CCC3)C(F)(F)F)cn(C3CCC3)c2c1] | B | D |
| 440 | 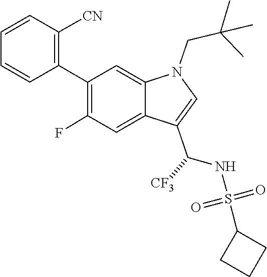 [SMILES: CC(C)(C)Cn1cc([C@H](NS(=O)(=O)C2CCC2)C(F)(F)F)c2cc(F)c(-c3ccccc3C#N)cc21] | A | D |
| 441 | 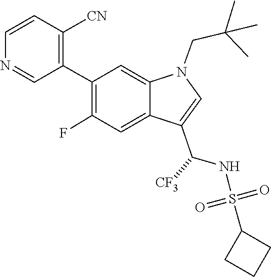 [SMILES: CC(C)(C)Cn1cc([C@H](NS(=O)(=O)C2CCC2)C(F)(F)F)c2cc(F)c(-c3cnccc3C#N)cc21] | A | D |
| 442 | 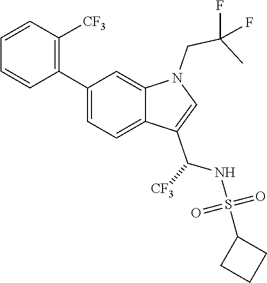 [SMILES: CC(F)(F)Cn1cc([C@H](NS(=O)(=O)C2CCC2)C(F)(F)F)c2ccc(-c3ccccc3C(F)(F)F)cc21] | B | D |
| 444 | 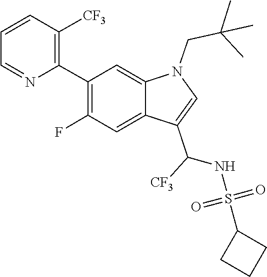 [SMILES: CC(C)(C)Cn1cc(C(NS(=O)(=O)C2CCC2)C(F)(F)F)c2cc(F)c(-c3ncccc3C(F)(F)F)cc21] | A | D |
| 445 | 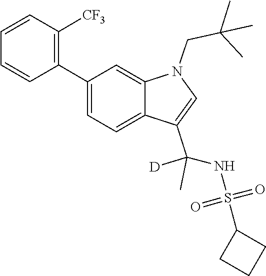 [SMILES: [2H]C(C)(NS(=O)(=O)C1CCC1)c1cn(CC(C)(C)C)c2cc(-c3ccccc3C(F)(F)F)ccc12] | A | D |
| 446 | 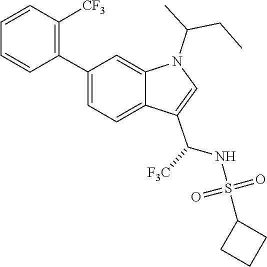 [SMILES: CCC(C)n1cc([C@H](NS(=O)(=O)C2CCC2)C(F)(F)F)c2ccc(-c3ccccc3C(F)(F)F)cc21] | A | D |
| 454 | 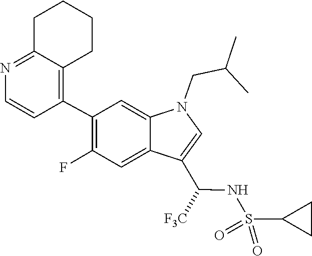 [SMILES: CC(C)Cn1cc([C@H](NS(=O)(=O)C2CC2)C(F)(F)F)c2cc(F)c(-c3ccnc4c3CCCC4)cc21] | A | D |
| 455 | 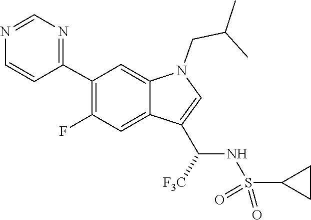 [SMILES: CC(C)Cn1cc([C@H](NS(=O)(=O)C2CC2)C(F)(F)F)c2cc(F)c(-c3ccncn3)cc21] | A | D |
| 456 | 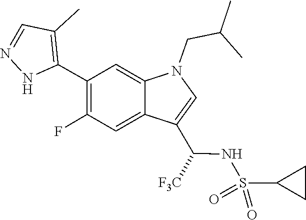 [SMILES: Cc1cn[nH]c1-c1cc2c(cc1F)c([C@H](NS(=O)(=O)C1CC1)C(F)(F)F)cn2CC(C)C] | B | D |
| 457 | 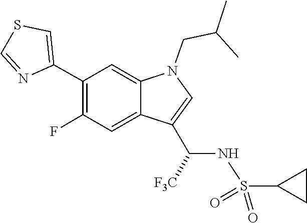 [SMILES: CC(C)Cn1cc([C@H](NS(=O)(=O)C2CC2)C(F)(F)F)c2cc(F)c(-c3cscn3)cc21] | B | D |
| 458 | 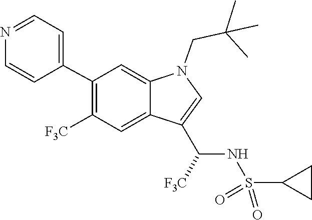 [SMILES: CC(C)(C)Cn1cc([C@H](NS(=O)(=O)C2CC2)C(F)(F)F)c2cc(C(F)(F)F)c(-c3ccncc3)cc21] | B | D |
| 459 | 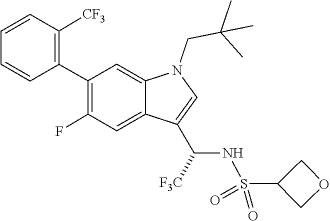 [SMILES: CC(C)(C)Cn1cc([C@H](NS(=O)(=O)C2COC2)C(F)(F)F)c2cc(F)c(-c3ccccc3C(F)(F)F)cc21] | A | D |
| 460 | 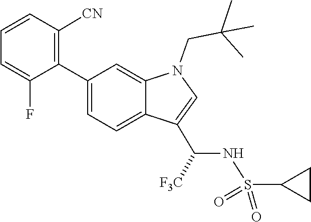 [SMILES: CC(C)(C)Cn1cc([C@H](NS(=O)(=O)C2CC2)C(F)(F)F)c2ccc(-c3c(F)cccc3C#N)cc21] | A | D |
| 461 | 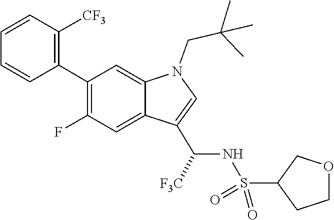 [SMILES: CC(C)(C)Cn1cc([C@H](NS(=O)(=O)C2CCOC2)C(F)(F)F)c2cc(F)c(-c3ccccc3C(F)(F)F)cc21] | C | D |
| 462 | 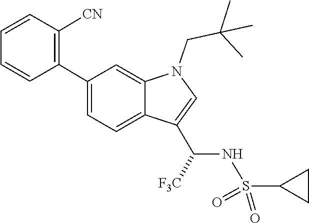 [SMILES: CC(C)(C)Cn1cc([C@H](NS(=O)(=O)C2CC2)C(F)(F)F)c2ccc(-c3ccccc3C#N)cc21] | A | D |
| 463 | 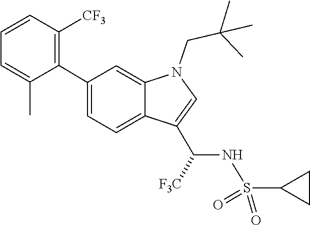 [SMILES: Cc1cccc(C(F)(F)F)c1-c1ccc2c([C@H](NS(=O)(=O)C3CC3)C(F)(F)F)cn(CC(C)(C)C)c2c1] | A | D |
| 464 | 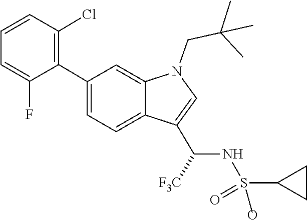 | A | D |
| 465 | 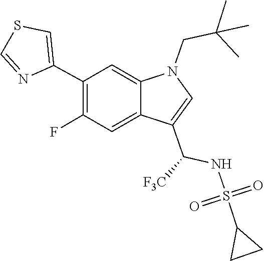 [SMILES: CC(C)(C)Cn1cc([C@H](NS(=O)(=O)C2CC2)C(F)(F)F)c2cc(F)c(-c3cscn3)cc21] | B | D |
| 466 | 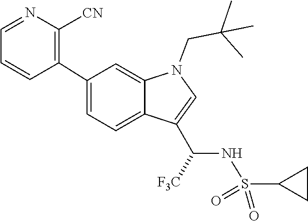 [SMILES: CC(C)(C)Cn1cc([C@H](NS(=O)(=O)C2CC2)C(F)(F)F)c2ccc(-c3cccnc3C#N)cc21] | A | D |
| 467 | 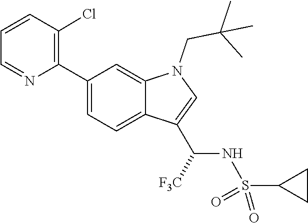 | B | D |
| 468 | 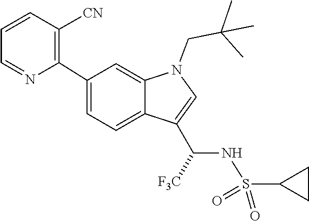 [SMILES: CC(C)(C)Cn1cc([C@H](NS(=O)(=O)C2CC2)C(F)(F)F)c2ccc(-c3ncccc3C#N)cc21] | A | D |
| 469 | 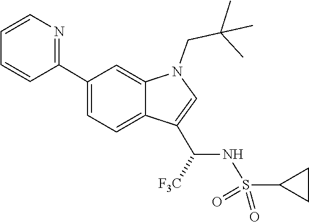 [SMILES: CC(C)(C)Cn1cc([C@H](NS(=O)(=O)C2CC2)C(F)(F)F)c2ccc(-c3ccccn3)cc21] | B | D |
| 470 | 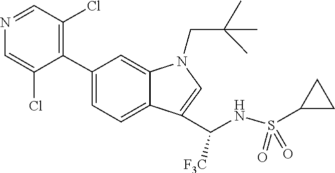 [SMILES: CC(C)(C)Cn1cc([C@H](NS(=O)(=O)C2CC2)C(F)(F)F)c2ccc(-c3c(Cl)cncc3Cl)cc21] | A | D |
| 471 | 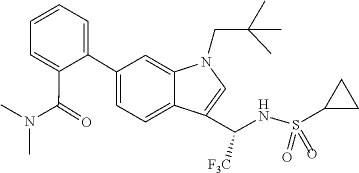 | B | D |
| 472 | 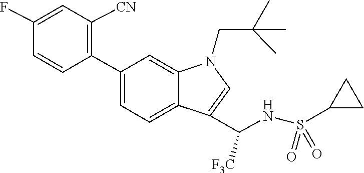 [SMILES: CC(C)(C)Cn1cc([C@H](NS(=O)(=O)C2CC2)C(F)(F)F)c2ccc(-c3ccc(F)cc3C#N)cc21] | A | D |
| 473 | 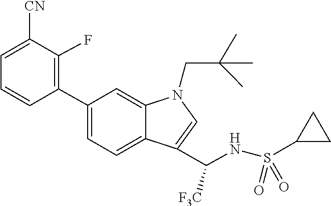 | B | D |
| 474 | 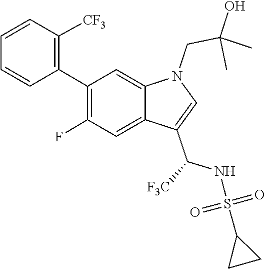 [SMILES: CC(C)(O)Cn1cc([C@H](NS(=O)(=O)C2CC2)C(F)(F)F)c2cc(F)c(-c3ccccc3C(F)(F)F)cc21] | B | D |
| 475 | 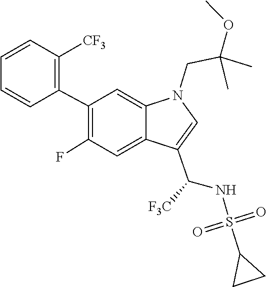 [SMILES: COC(C)(C)Cn1cc([C@H](NS(=O)(=O)C2CC2)C(F)(F)F)c2cc(F)c(-c3ccccc3C(F)(F)F)cc21] | A | D |
| 476 | 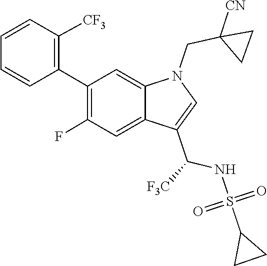 [SMILES: N#CC1(Cn2cc([C@H](NS(=O)(=O)C3CC3)C(F)(F)F)c3cc(F)c(-c4ccccc4C(F)(F)F)cc32)CC1] | B | D |
| 477 | 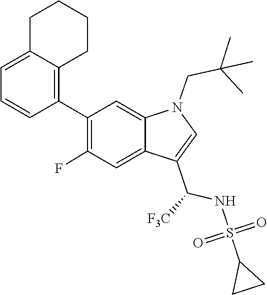 [SMILES: CC(C)(C)Cn1cc([C@H](NS(=O)(=O)C2CC2)C(F)(F)F)c2cc(F)c(-c3cccc4c3CCCC4)cc21] | B | D |
| 478 | 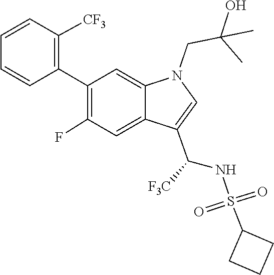 [SMILES: CC(C)(O)Cn1cc([C@H](NS(=O)(=O)C2CCC2)C(F)(F)F)c2cc(F)c(-c3ccccc3C(F)(F)F)cc21] | B | D |
| 479 | 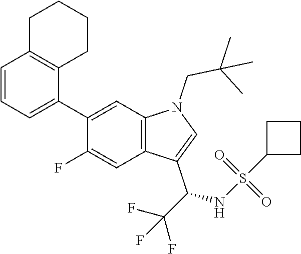 [SMILES: CC(C)(C)Cn1cc([C@H](NS(=O)(=O)C2CCC2)C(F)(F)F)c2cc(F)c(-c3cccc4c3CCCC4)cc21] | A | D |
| 480 | 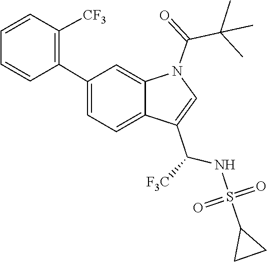 [SMILES: CC(C)(C)C(=O)n1cc([C@H](NS(=O)(=O)C2CC2)C(F)(F)F)c2ccc(-c3ccccc3C(F)(F)F)cc21] | B | D |
| 481 | 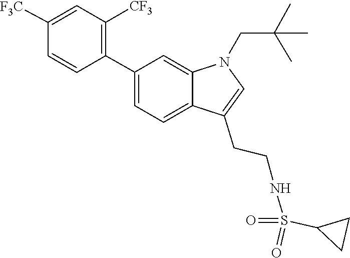 [SMILES: CC(C)(C)Cn1cc(CCNS(=O)(=O)C2CC2)c2ccc(-c3ccc(C(F)(F)F)cc3C(F)(F)F)cc21] | B | D |
| 482 | 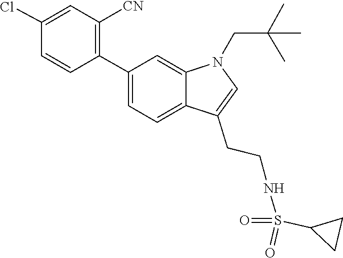 [SMILES: CC(C)(C)Cn1cc(CCNS(=O)(=O)C2CC2)c2ccc(-c3ccc(Cl)cc3C#N)cc21] | A | D |
| 483 | 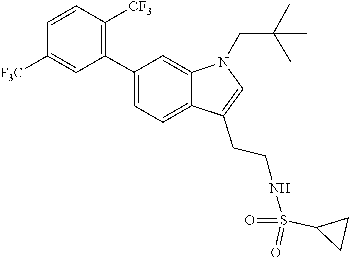 [SMILES: CC(C)(C)Cn1cc(CCNS(=O)(=O)C2CC2)c2ccc(-c3cc(C(F)(F)F)ccc3C(F)(F)F)cc21] | A | D |
| 484 | 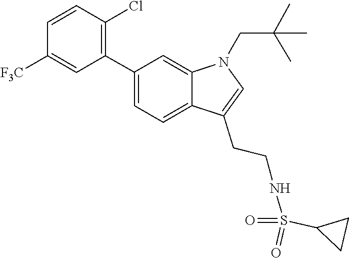 [SMILES: CC(C)(C)Cn1cc(CCNS(=O)(=O)C2CC2)c2ccc(-c3cc(C(F)(F)F)ccc3Cl)cc21] | A | D |
| 485 | 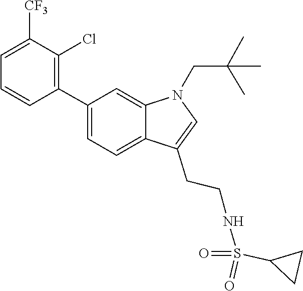 [SMILES: CC(C)(C)Cn1cc(CCNS(=O)(=O)C2CC2)c2ccc(-c3cccc(C(F)(F)F)c3Cl)cc21] | B | D |
| 486 | 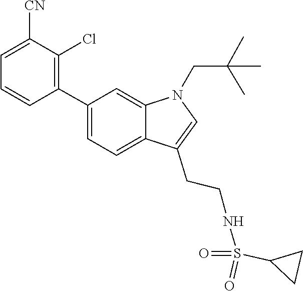 [SMILES: CC(C)(C)Cn1cc(CCNS(=O)(=O)C2CC2)c2ccc(-c3cccc(C#N)c3Cl)cc21] | B | D |
| 487 | 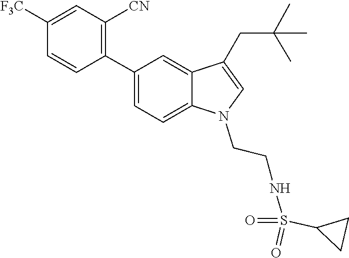 [SMILES: CC(C)(C)Cc1cn(CCNS(=O)(=O)C2CC2)c2ccc(-c3ccc(C(F)(F)F)cc3C#N)cc12] | B | D |
| 488 | 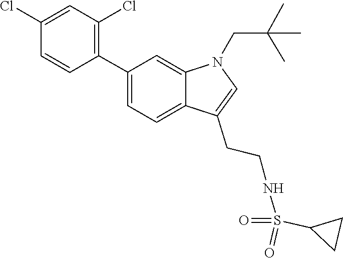 [SMILES: CC(C)(C)Cn1cc(CCNS(=O)(=O)C2CC2)c2ccc(-c3ccc(Cl)cc3Cl)cc21] | A | D |
| 489 | 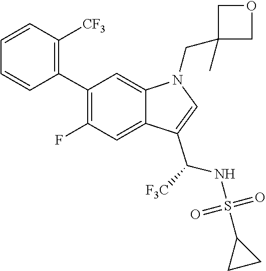 [SMILES: CC1(Cn2cc([C@H](NS(=O)(=O)C3CC3)C(F)(F)F)c3cc(F)c(-c4ccccc4C(F)(F)F)cc32)COC1] | A | D |
| 490 | 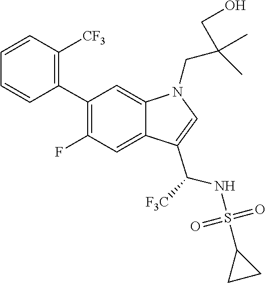 [SMILES: CC(C)(CO)Cn1cc([C@H](NS(=O)(=O)C2CC2)C(F)(F)F)c2cc(F)c(-c3ccccc3C(F)(F)F)cc21] | B | D |
| 491 | 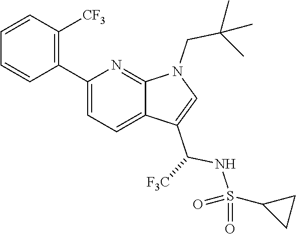 [SMILES: CC(C)(C)Cn1cc([C@H](NS(=O)(=O)C2CC2)C(F)(F)F)c2ccc(-c3ccccc3C(F)(F)F)nc21] | A | D |
| 492 | 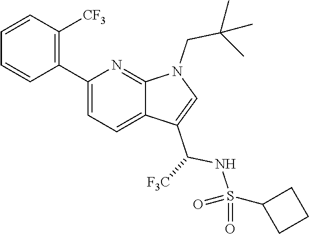 [SMILES: CC(C)(C)Cn1cc([C@H](NS(=O)(=O)C2CCC2)C(F)(F)F)c2ccc(-c3ccccc3C(F)(F)F)nc21] | A | D |
| 493 | 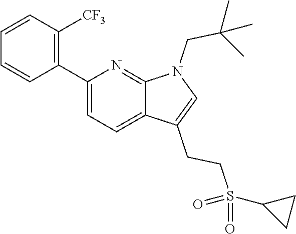 [SMILES: CC(C)(C)Cn1cc(CCS(=O)(=O)C2CC2)c2ccc(-c3ccccc3C(F)(F)F)nc21] | A | D |
| 494 | 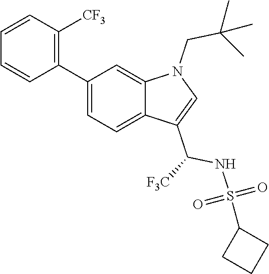 [SMILES: CC(C)(C)Cn1cc([C@H](NS(=O)(=O)C2CCC2)C(F)(F)F)c2ccc(-c3ccccc3C(F)(F)F)cc21] | A | D |
| 495 | 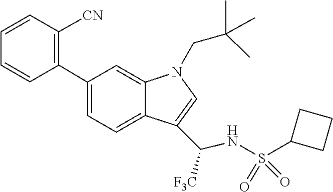 [SMILES: CC(C)(C)Cn1cc([C@H](NS(=O)(=O)C2CCC2)C(F)(F)F)c2ccc(-c3ccccc3C#N)cc21] | A | D |
| 496 | 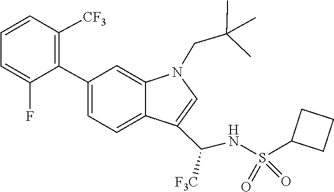 [SMILES: CC(C)(C)Cn1cc([C@H](NS(=O)(=O)C2CCC2)C(F)(F)F)c2ccc(-c3c(F)cccc3C(F)(F)F)cc21] | A | D |
| 497 | 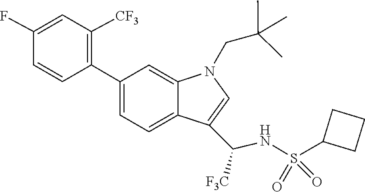 [SMILES: CC(C)(C)Cn1cc([C@H](NS(=O)(=O)C2CCC2)C(F)(F)F)c2ccc(-c3ccc(F)cc3C(F)(F)F)cc21] | A | D |
| 498 | 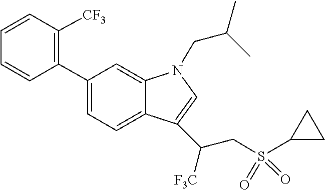 [SMILES: CC(C)Cn1cc(C(CS(=O)(=O)C2CC2)C(F)(F)F)c2ccc(-c3ccccc3C(F)(F)F)cc21] | B | D |
| 499 | 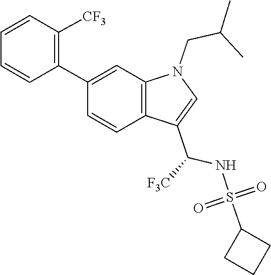 [SMILES: CC(C)Cn1cc([C@H](NS(=O)(=O)C2CCC2)C(F)(F)F)c2ccc(-c3ccccc3C(F)(F)F)cc21] | A | D |
| 500 | 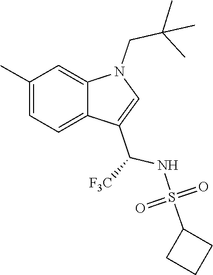 [SMILES: Cc1ccc2c([C@H](NS(=O)(=O)C3CCC3)C(F)(F)F)cn(CC(C)(C)C)c2c1] | A | D |
| 501 | 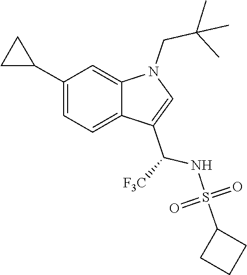 [SMILES: CC(C)(C)Cn1cc([C@H](NS(=O)(=O)C2CCC2)C(F)(F)F)c2ccc(C3CC3)cc21] | A | D |
| 502 | 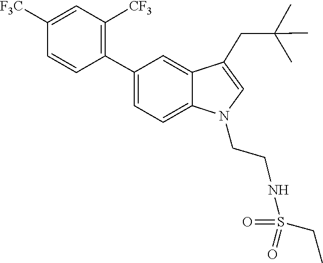 [SMILES: CCS(=O)(=O)NCCn1cc(CC(C)(C)C)c2cc(-c3ccc(C(F)(F)F)cc3C(F)(F)F)ccc21] | A | D |
| 503 | 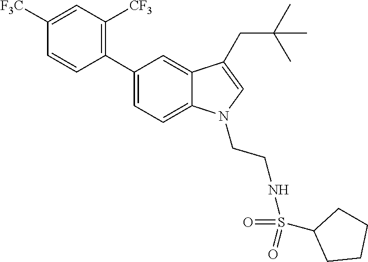 [SMILES: CC(C)(C)Cc1cn(CCNS(=O)(=O)C2CCCC2)c2ccc(-c3ccc(C(F)(F)F)cc3C(F)(F)F)cc12] | A | D |
| 504 | 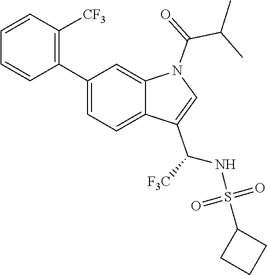 [SMILES: CC(C)C(=O)n1cc([C@H](NS(=O)(=O)C2CCC2)C(F)(F)F)c2ccc(-c3ccccc3C(F)(F)F)cc21] | A | D |
| 505 | 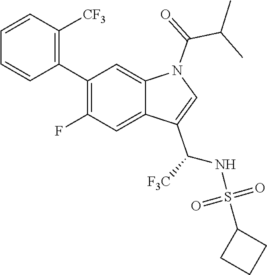 [SMILES: CC(C)C(=O)n1cc([C@H](NS(=O)(=O)C2CCC2)C(F)(F)F)c2cc(F)c(-c3ccccc3C(F)(F)F)cc21] | A | D |
| 506 | 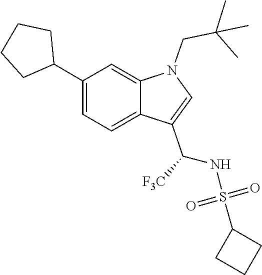 [SMILES: CC(C)(C)Cn1cc([C@H](NS(=O)(=O)C2CCC2)C(F)(F)F)c2ccc(C3CCCC3)cc21] | A | D |
| 507 | 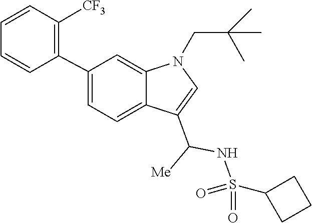 [SMILES: CC(NS(=O)(=O)C1CCC1)c1cn(CC(C)(C)C)c2cc(-c3ccccc3C(F)(F)F)ccc12] | A | D |
| 508 | 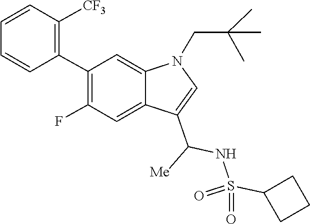 [SMILES: CC(NS(=O)(=O)C1CCC1)c1cn(CC(C)(C)C)c2cc(-c3ccccc3C(F)(F)F)c(F)cc12] | A | D |
| 509 | 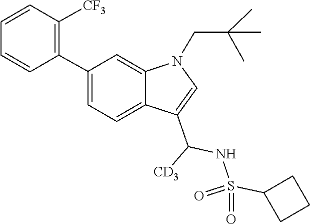 [SMILES: [2H]C([2H])([2H])C(NS(=O)(=O)C1CCC1)c1cn(CC(C)(C)C)c2cc(-c3ccccc3C(F)(F)F)ccc12] | A | D |
| 510 | 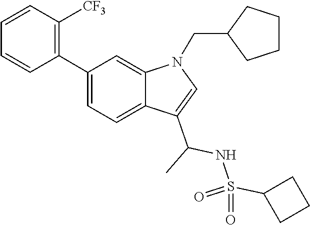 [SMILES: CC(NS(=O)(=O)C1CCC1)c1cn(CC2CCCC2)c2cc(-c3ccccc3C(F)(F)F)ccc12] | A | D |
| 511 | 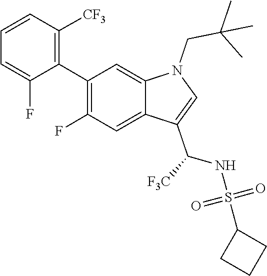 [SMILES: CC(C)(C)Cn1cc([C@H](NS(=O)(=O)C2CCC2)C(F)(F)F)c2cc(F)c(-c3c(F)cccc3C(F)(F)F)cc21] | B | D |
| 512 | 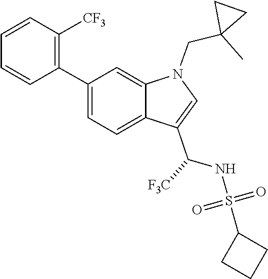 [SMILES: CC1(Cn2cc([C@H](NS(=O)(=O)C3CCC3)C(F)(F)F)c3ccc(-c4ccccc4C(F)(F)F)cc32)CC1] | A | D |
| 513 | 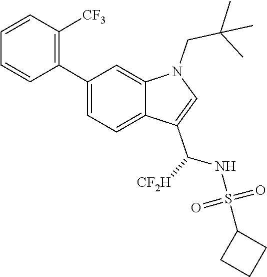 [SMILES: CC(C)(C)Cn1cc([C@H](NS(=O)(=O)C2CCC2)C(F)F)c2ccc(-c3ccccc3C(F)(F)F)cc21] | A | D |
| 514 | 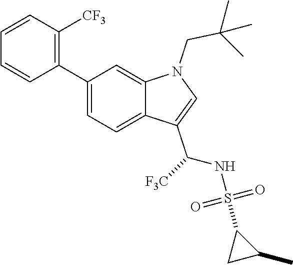 [SMILES: C[C@H]1C[C@@H]1S(=O)(=O)N[C@@H](c1cn(CC(C)(C)C)c2cc(-c3ccccc3C(F)(F)F)ccc12)C(F)(F)F] | A | D |
| 515 | 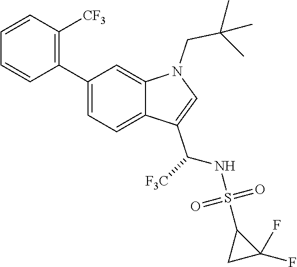 [SMILES: CC(C)(C)Cn1cc([C@H](NS(=O)(=O)C2CC2(F)F)C(F)(F)F)c2ccc(-c3ccccc3C(F)(F)F)cc21] | B | D |
| 516 | 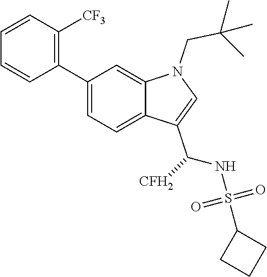 | A | D |
| 517 | 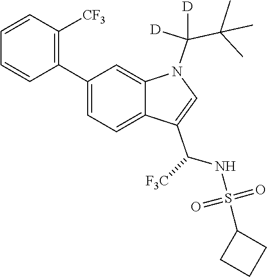 [SMILES: [2H]C([2H])(n1cc([C@H](NS(=O)(=O)C2CCC2)C(F)(F)F)c2ccc(-c3ccccc3C(F)(F)F)cc21)C(C)(C)C] | A | D |
| 518 | 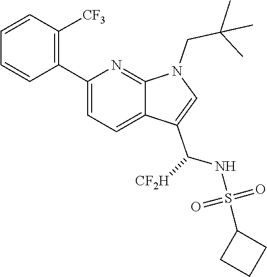 [SMILES: CC(C)(C)Cn1cc([C@H](NS(=O)(=O)C2CCC2)C(F)F)c2ccc(-c3ccccc3C(F)(F)F)nc21] | A | D |
| 519 | 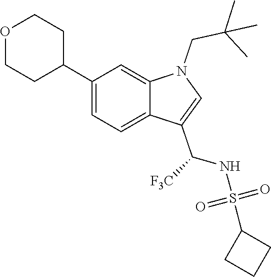 [SMILES: CC(C)(C)Cn1cc([C@H](NS(=O)(=O)C2CCC2)C(F)(F)F)c2ccc(C3CCOCC3)cc21] | A | D |
| 520 | 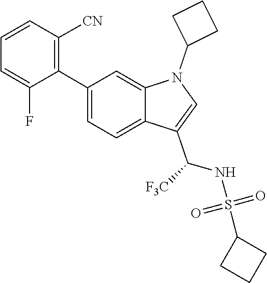 [SMILES: N#Cc1cccc(F)c1-c1ccc2c([C@H](NS(=O)(=O)C3CCC3)C(F)(F)F)cn(C3CCC3)c2c1] | A | D |
| 521 | 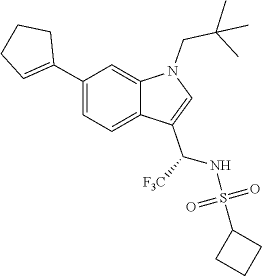 [SMILES: CC(C)(C)Cn1cc([C@H](NS(=O)(=O)C2CCC2)C(F)(F)F)c2ccc(C3=CCCC3)cc21] | A | D |
| 522 | 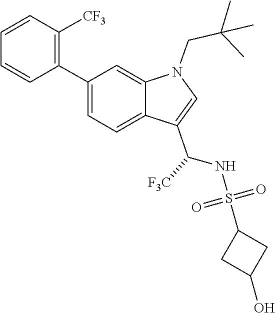 [SMILES: CC(C)(C)Cn1cc([C@H](NS(=O)(=O)C2CC(O)C2)C(F)(F)F)c2ccc(-c3ccccc3C(F)(F)F)cc21] | B | D |
| 523 | 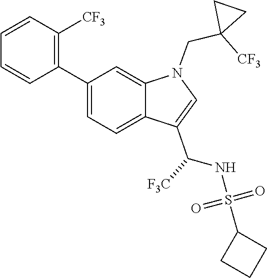 [SMILES: O=S(=O)(N[C@@H](c1cn(CC2(C(F)(F)F)CC2)c2cc(-c3ccccc3C(F)(F)F)ccc12)C(F)(F)F)C1CCC1] | A | D |
| 524 | 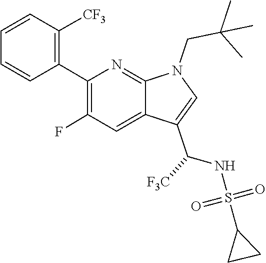 [SMILES: CC(C)(C)Cn1cc([C@H](NS(=O)(=O)C2CC2)C(F)(F)F)c2cc(F)c(-c3ccccc3C(F)(F)F)nc21] | A | D |
| 525 | 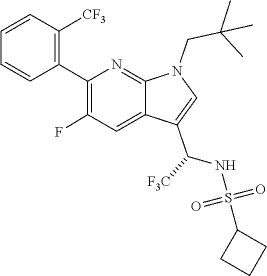 [SMILES: CC(C)(C)Cn1cc([C@H](NS(=O)(=O)C2CCC2)C(F)(F)F)c2cc(F)c(-c3ccccc3C(F)(F)F)nc21] | A | D |
| 526 | 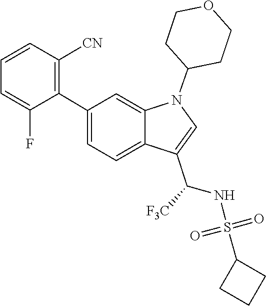 [SMILES: N#Cc1cccc(F)c1-c1ccc2c([C@H](NS(=O)(=O)C3CCC3)C(F)(F)F)cn(C3CCOCC3)c2c1] | A | D |
| 527 | 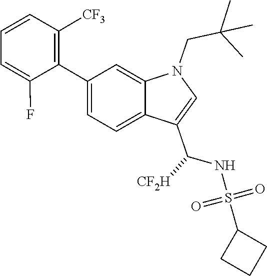 [SMILES: CC(C)(C)Cn1cc([C@H](NS(=O)(=O)C2CCC2)C(F)F)c2ccc(-c3c(F)cccc3C(F)(F)F)cc21] | A | D |
| 528 | 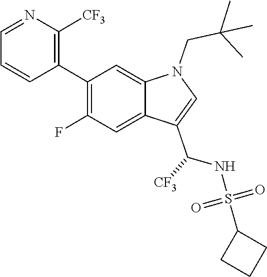 [SMILES: CC(C)(C)Cn1cc([C@H](NS(=O)(=O)C2CCC2)C(F)(F)F)c2cc(F)c(-c3cccnc3C(F)(F)F)cc21] | A | D |
| 529 | 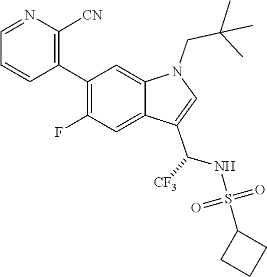 [SMILES: CC(C)(C)Cn1cc([C@H](NS(=O)(=O)C2CCC2)C(F)(F)F)c2cc(F)c(-c3cccnc3C#N)cc21] | A | D |
| 530 | 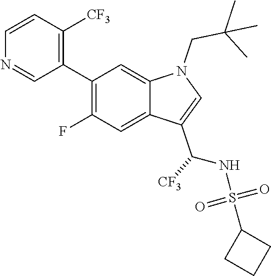 [SMILES: CC(C)(C)Cn1cc([C@H](NS(=O)(=O)C2CCC2)C(F)(F)F)c2cc(F)c(-c3cnccc3C(F)(F)F)cc21] | A | D |
| 533 | 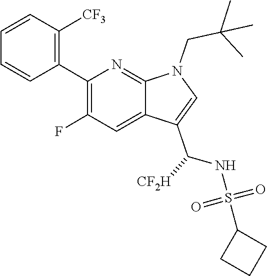 [SMILES: CC(C)(C)Cn1cc([C@H](NS(=O)(=O)C2CCC2)C(F)F)c2cc(F)c(-c3ccccc3C(F)(F)F)nc21] | A | D |
| 534 | 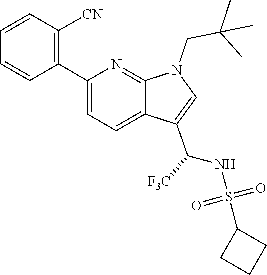 [SMILES: CC(C)(C)Cn1cc([C@H](NS(=O)(=O)C2CCC2)C(F)(F)F)c2ccc(-c3ccccc3C#N)nc21] | A | D |
| 535 | 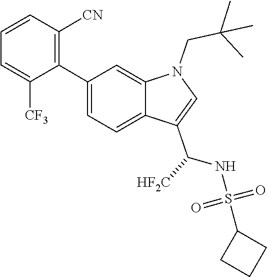 | A | D |
| 536 | 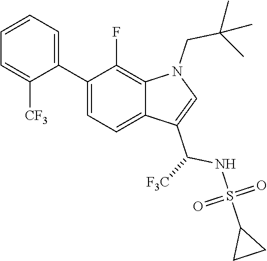 [SMILES: CC(C)(C)Cn1cc([C@H](NS(=O)(=O)C2CC2)C(F)(F)F)c2ccc(-c3ccccc3C(F)(F)F)c(F)c21] | A | D |
| 537 | 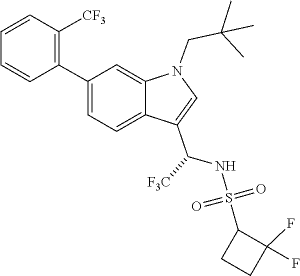 [SMILES: CC(C)(C)Cn1cc([C@H](NS(=O)(=O)C2CCC2(F)F)C(F)(F)F)c2ccc(-c3ccccc3C(F)(F)F)cc21] | C | D |
| 538 | 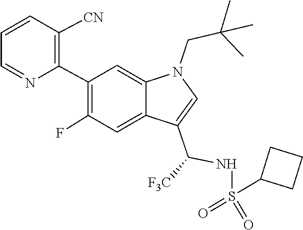 [SMILES: CC(C)(C)Cn1cc([C@H](NS(=O)(=O)C2CCC2)C(F)(F)F)c2cc(F)c(-c3ncccc3C#N)cc21] | A | D |
| 541 | 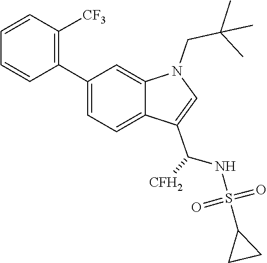 | A | D |
| 542 | 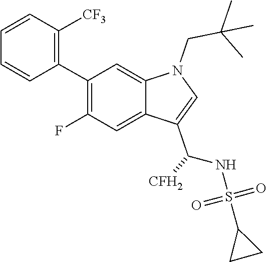 | A | D |
| 543 | 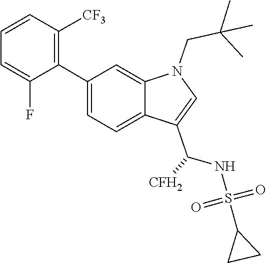 | A | D |
| 544 | 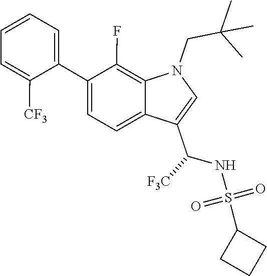 [SMILES: CC(C)(C)Cn1cc([C@H](NS(=O)(=O)C2CCC2)C(F)(F)F)c2ccc(-c3ccccc3C(F)(F)F)c(F)c21] | A | D |
| 545 | 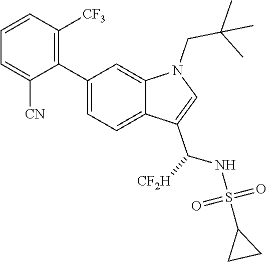 [SMILES: CC(C)(C)Cn1cc([C@H](NS(=O)(=O)C2CC2)C(F)F)c2ccc(-c3c(C#N)cccc3C(F)(F)F)cc21] | A | D |
| 546 | 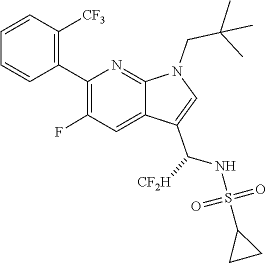 [SMILES: CC(C)(C)Cn1cc([C@H](NS(=O)(=O)C2CC2)C(F)F)c2cc(F)c(-c3ccccc3C(F)(F)F)nc21] | A | D |
| 547 | 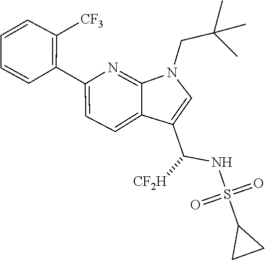 [SMILES: CC(C)(C)Cn1cc([C@H](NS(=O)(=O)C2CC2)C(F)F)c2ccc(-c3ccccc3C(F)(F)F)nc21] | A | D |
| 550 | 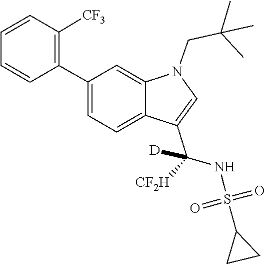 [SMILES: [2H][C@](NS(=O)(=O)C1CC1)(c1cn(CC(C)(C)C)c2cc(-c3ccccc3C(F)(F)F)ccc12)C(F)F] | A | D |
| 551 | 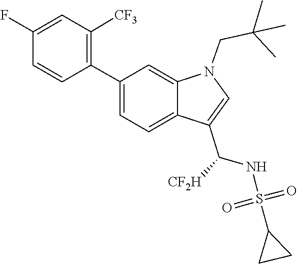 [SMILES: CC(C)(C)Cn1cc([C@H](NS(=O)(=O)C2CC2)C(F)F)c2ccc(-c3ccc(F)cc3C(F)(F)F)cc21] | A | D |
| 552 | 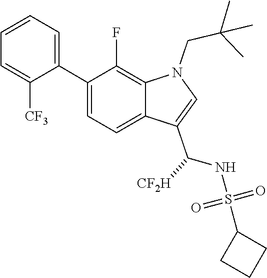 [SMILES: CC(C)(C)Cn1cc([C@H](NS(=O)(=O)C2CCC2)C(F)F)c2ccc(-c3ccccc3C(F)(F)F)c(F)c21] | A | D |
| 553 | 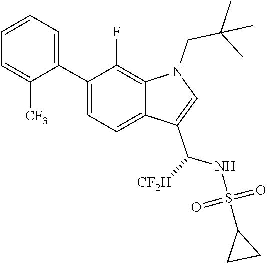 [SMILES: CC(C)(C)Cn1cc([C@H](NS(=O)(=O)C2CC2)C(F)F)c2ccc(-c3ccccc3C(F)(F)F)c(F)c21] | A | D |
| 562 | 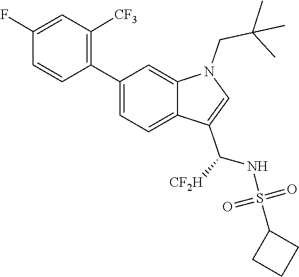 [SMILES: CC(C)(C)Cn1cc([C@H](NS(=O)(=O)C2CCC2)C(F)F)c2ccc(-c3ccc(F)cc3C(F)(F)F)cc21] | A | D |
| 563 | 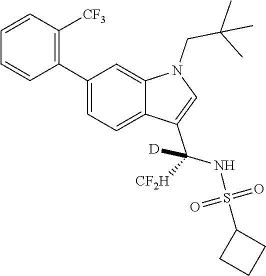 [SMILES: [2H][C@](NS(=O)(=O)C1CCC1)(c1cn(CC(C)(C)C)c2cc(-c3ccccc3C(F)(F)F)ccc12)C(F)F] | A | D |
| 564 | 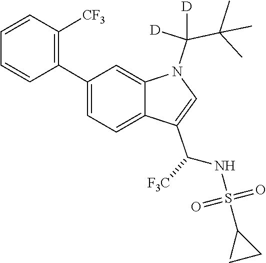 [SMILES: [2H]C([2H])(n1cc([C@H](NS(=O)(=O)C(C)CC)C(F)(F)F)c2ccc(-c3ccccc3C(F)(F)F)cc21)C(C)(C)C] | A | D |

We claim:

1. A compound of Formula IA or IB: 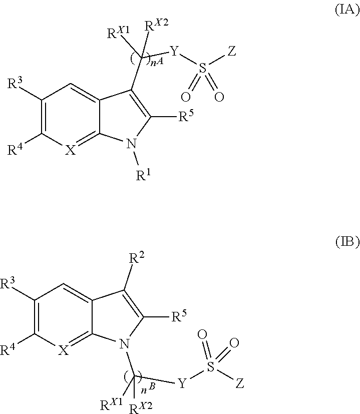
wherein
X is CH, CCl, CF, CBr, C(C₁-C₆-alkyl), or N;
R¹ is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S),
R² is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —C(O)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S), and
wherein any alkyl, alkoxy, aryl, cycloalkyl, and heteroaryl in R¹ and R² is optionally substituted with 1 to 6 substituents selected from the group consisting of halo, NO₂, OH, CN, and C₁-C₆-haloalkyl;
R^{X1} and R^{X2} are independently selected from the group consisting of H, C₁-C₆-alkyl, C₁-C₆-haloalkyl, C₁-C₆-hydroxyalkyl, C₃-C₅-cycloalkyl, and —C(O)O(R′)₂ (wherein each R′ is independently selected from H and C₁-C₆-alkyl),
or R^{X1} and R^{X2} together with the carbon atom to which they are bound form a spiro-fused C₃-C₅-cycloalkyl;
or any two vicinal R^{X1} and R^{X2} together with the carbon atoms to which they are bound form a C₃-C₅-cycloalkyl or a moiety selected from 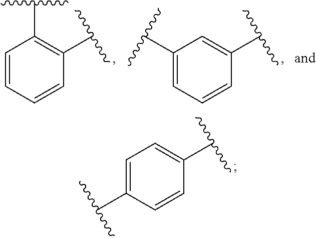
Y is —CR^{Y1}R^{Y2}— or —NR^{Y1}—;
R^{Y1} and R^{Y2} are independently selected from H and C₁-C₆-alkyl;
n^{A} is 0, 1, or 2;
n^{B} is 1 or 2;
when Y is —NR^{Y1}—, then n^{A} is 1 or 2 and, n^{B} is 2 and, optionally, R^{Y1} and one of R^{X1} and R^{X2}, together with the atoms to which they are bound, form a 3- to 6-membered heterocycloalkyl (wherein 1-4 ring members are independently selected from N, O, and S);
Z is selected from the group consisting of C₁-C₆-alkyl, C₁-C₆-haloalkyl, C₃-C₅-cycloalkyl, 5- to 10-membered heteroaryl (wherein 1-4 heteroaryl members are independently selected from N, O, and S), 3- to 6-membered heterocycloalkyl(wherein 1-4 ring members are independently selected from N, O, and S); and —NR^{Z1}R^{Z2};
R^{Z1} and R^{Z2} are independently selected from the group consisting of H, C₁-C₆-alkyl and C₃-C₈-cycloalkyl,
or R^{Z1} and R^{Z2} together with the nitrogen to which they are bound form a 3- to 6-membered heterocycloalkyl (wherein 1-4 ring members are independently selected from N, O, and S);
wherein any alkyl, aryl, cycloalkyl, heterocycloalkyl, and heteroaryl in Z is optionally substituted with 1 to 6 substituents selected from the group consisting of halo, NO₂, OH, and C₁-C₆-haloalkyl;
R³ and R⁴ are independently selected from the group consisting of H, halo, CN, OH, N(R′)₂ (wherein each R′ is independently selected from H and C₁-C₆-alkyl), C₁-C₆-alkyl, C₁-C₆-haloalkyl, C₂-C₆-alkenyl, C₂-C₆-alkynyl, C₁-C₆-alkoxy, —O(C₁-C₆-alkyl)(C₆-C₁₀-aryl), C₃-C₈-cycloalkyl, C₆-C₁₀-aryl, and 5- to 10-membered heteroaryl (wherein 1-4 heteroaryl members are independently selected from N, O, and S), and 3- to 6-membered heterocycloalkyl (wherein 1-4 ring members are independently selected from N, O, and S); wherein
R³ and R⁴ are not simultaneously H or simultaneously any combination of aryl, heteroaryl, and cycloalkyl; and
R³ and R⁴ are optionally substituted with 1 to 3 substituents selected from the group consisting of halo, NO₂, OH, CN, C₁-C₆-alkyl, C₁-C₆-hydroxyalkyl, C₁-C₆-haloalkyl, —C₁-C₆-haloalkyl(OH), C₁-C₆-alkoxy, —S(O)₂C₁-C₆-alkyl, —S(O)₂NH(C₁-C₆-alkyl), —C(O)(C₁-C₆-alkyl), —C(O)O(C₁-C₆-alkyl), —C(O)N(R′) (wherein each R′ is independently selected from H and C₁-C₆-alkyl), —NHC(O)R′, 3- to 6-membered heterocycloalkyl (wherein 1-4 ring members are independently selected from N, O, and S), and C₆-C₁₀-aryl;
or R³ and R⁴, together with the carbon atoms to which they are bound, form a fused C₅-C₆-cycloalkyl ring optionally substituted as defined for R³ and R⁴ above;
R⁵ is selected from H and C₁-C₆-alkyl;
or a pharmaceutically acceptable salt thereof;
and wherein the compound is not any of the following:

| | |
|---|---|
| dimethyl({2-[3-(2-methylpropyl)-5-(pyridin- 4-yl)-1H-pyrrolo[2,3-b]pyridin-1- yl]ethyl}sulfamoyl)amine | 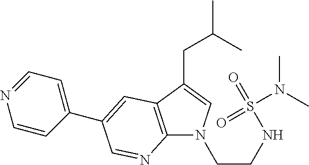 [SMILES: CC(C)Cc1cn(CCNS(=O)(=O)N(C)C)c2ncc(-c3ccncc3)cc12] |
| N-[2-(1,6-dimethyl-1H-indol-3-yl)ethyl]-3- methyl-1-piperidinesulfonamide | 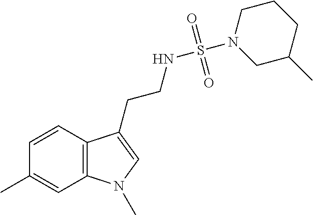 |
| N-[2-(1,6-dimethyl-1H-indol-3-yl)ethyl]-4- methyl-1-piperidinesulfonamide | 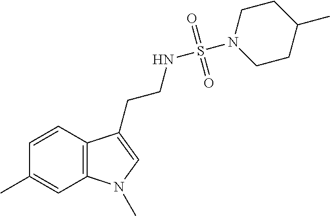 |
| N-[2-(1,6-dimethyl-1H-indol-3-yl)ethyl]-1- piperidinesulfonamide | 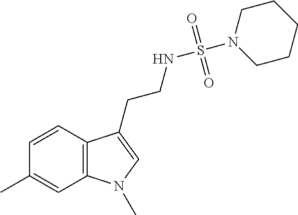 [SMILES: Cc1ccc2c(CCNS(=O)(=O)N3CCCCC3)cn(C)c2c1] |
| N-[2-(1,6-dimethyl-1H-indol-3-yl)ethyl]-1- pyrrolidinesulfonamide | 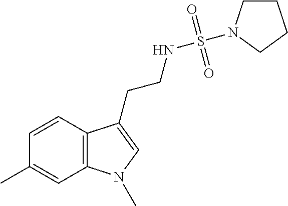 [SMILES: Cc1ccc2c(CCNS(=O)(=O)N3CCCC3)cn(C)c2c1] |
| N-[2-(1,6-dimethyl-1H-indol-3-yl)ethyl]-3- methyl-1-pyrrolidinesulfonamide | 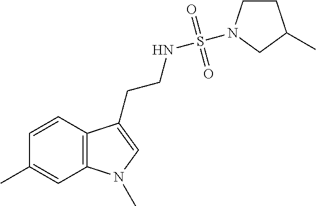 |
| N-[2-(1,6-dimethyl-1H-indol-3-yl)ethyl]-2- methyl-1-piperidinesulfonamide | 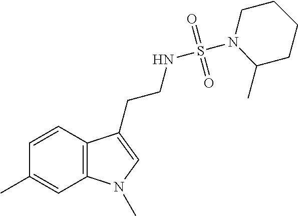 [SMILES: Cc1ccc2c(CCNS(=O)(=O)N3CCCCC3C)cn(C)c2c1] |
| N-[2-(1,6-dimethyl-1H-indol-3-yl)ethyl]-2- azabicyclo[2.2.1]heptane-2-sulfonamide | 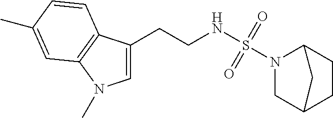 [SMILES: Cc1ccc2c(CCNS(=O)(=O)N3CC4CCC3C4)cn(C)c2c1] |
| N-[2-(1,6-dimethyl-1H-indol-3-yl)ethy1]-2- methyl-1-pyrrolidinesulfonamide | 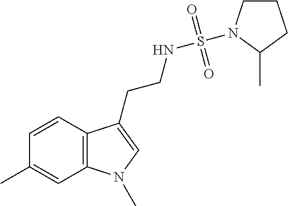 [SMILES: Cc1ccc2c(CCNS(=O)(=O)N3CCCC3C)cn(C)c2c1] |
| N-[2-(1,6-dimethyl-1H-indol-3-yl)ethyl]-4- morpholinesulfonamide | 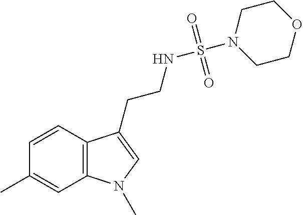 [SMILES: Cc1ccc2c(CCNS(=O)(=O)N3CCOCC3)cn(C)c2c1] |
| N-[2-(1,6-dimethyl-1H-indol-3-yl)ethyl]-2,6- dimethyl-4-morpholinesulfonamide | 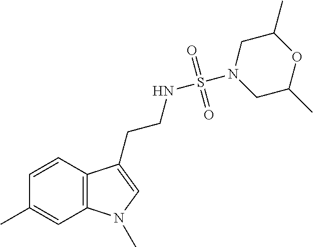 |
| N-[2-(1,6-dimethyl-1H-indol-3-yl)ethyl]-2,2- dimethyl-4-morpholinesulfonamide | 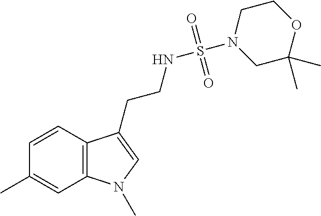 |
| 1-[[[2-(1,6-dimethyl-1H-indol-3- ylethyl]amino]sulfonyl]-4- Piperidinecarboxylic acid methyl ester | 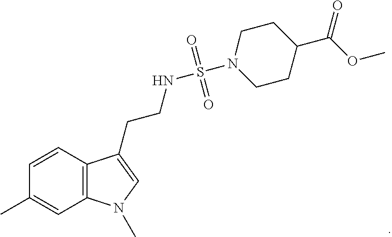 |

2. The compound according to claim 1, wherein:
X is CH, CCl, CF, CBr, C(C₁-C₆-alkyl), or N;
R¹ is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S),
R² is selected from the group consisting of C₁-C₆-alkyl, C₁-C₆-haloalkyl, —(C₁-C₆-alkyl)(C₁-C₆-alkoxy), —C(O)(C₁-C₆-alkyl), —C(O)O(C₁-C₆-alkyl), —C(O)(C₆-C₁₀-aryl), —SO₂(C₁-C₆-alkyl), —(C₁-C₆-alkyl)C(O)O(C₁-C₆-alkyl), —(C₁-C₆-alkyl)N(R′)₂, —C(O)N(R′)₂, —(C₁-C₆-alkyl)C(O)N(R′)₂ (wherein each R′ is independently selected from H and C₁-C₆-alkyl), C₃-C₈-cycloalkyl, —(C₁-C₆-alkyl)(C₆-C₁₀-aryl), —(C₁-C₆-alkyl)(C₃-C₈-cycloalkyl), —(C₁-C₆-alkyl)(3- to 6-membered heterocycloalkyl (wherein 1-4 ring members are independently selected from N, O, and S), and
wherein any alkyl, alkoxy, aryl, cycloalkyl, and heteroaryl in R¹ and R² is optionally substituted with 1 to 6 substituents selected from the group consisting of halo, NO₂, OH, C₁-C₆-haloalkyl;
R^{X1} and R^{X2} are independently selected from the group consisting of H, C₁-C₆-alkyl, and C₁-C₆-haloalkyl,
or R^{X1} and R^{X2} together with the carbon atom to which they are bound form a spiro-fused C₃-C₅-cycloalkyl;
or any two vicinal R^{X1} and R^{X2} together with the carbon atoms to which they are bound form a C₃-C₅-cycloalkyl or a moiety selected from 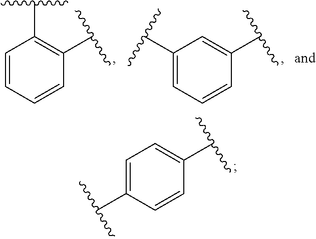
Y is —CR^{Y1}R^{Y2}— or —NR^{Y1}—;
R^{Y1} and R^{Y2} are independently selected from H and C₁-C₆-alkyl;
n^{A} is 0, 1, or 2;
n^{B} is 1 or 2;
when Y is —NR^{Y1}—, then n^{A} is 1 or 2 and, n^{B} is 2 and, optionally, R^{Y1} and one of R^{X1} and R^{X2}, together with the atoms to which they are bound, form a 3- to 6-membered heterocycloalkyl (wherein 1-4 ring members are independently selected from N, O, and S);
Z is selected from the group consisting of C₁-C₆-alkyl, C₁-C₆-haloalkyl, C₃-C₅-cycloalkyl, 5- to 10-membered heteroaryl (wherein 1-4 heteroaryl members are independently selected from N, O, and S), 3- to 6-membered heterocycloalkyl(wherein 1-4 ring members are independently selected from N, O, and S); and —NR^{Z1}R^{Z2};
R^{Z1} and R^{Z2} are independently selected from the group consisting of H, C₁-C₆-alkyl and C₃-C₈-cycloalkyl,
or R^{Z1} and R^{Z2} together with the nitrogen to which they are bound form a 3- to 6-membered heterocycloalkyl (wherein 1-4 ring members are independently selected from N, O, and S);
wherein any alkyl, aryl, cycloalkyl, heterocycloalkyl, and heteroaryl in Z is optionally substituted with 1 to 6 substituents selected from the group consisting of halo, NO₂, OH, and C₁-C₆-haloalkyl;
R³ and R⁴ are independently selected from the group consisting of H, halo, CN, OH N(R′)₂ (wherein each R′ is independently selected from H and C₁-C₆ alkyl), C₁-C₆-alkyl, C₁-C₆haloalkyl, C₂-C₆-alkenyl, C₂-C₆-alkynyl, C₁-C₆-alkoxy, —O(C₁-C₆-alkyl)(C₆-C₁₀-aryl), C₃-C₈-cycloalkyl, C₆-C₁₀-aryl, and 5- to 10-membered heteroaryl (wherein 1-4 heteroaryl members are independently selected from N, O, and S); wherein
R³ and R⁴ are not simultaneously H or simultaneously any combination of aryl, heteroaryl, and cycloalkyl;
R³ and R⁴ are optionally substituted with 1 to 3 substituents selected from the group consisting of halo, NO₂, OH, C₁-C₆-haloalkyl, —C₁-C₆-haloalkyl(OH), —C(O)(C₁-C₆-alkyl), —C(O)O(C₁-C₆-alkyl);
R⁵ is selected from H and C₁-C₆-alkyl;
or a pharmaceutically acceptable salt thereof.

3. The compound according to claim 1 or 2, wherein X is CH.

4. The compound according to claim 1 or 2, wherein the compound is of Formula IA, and R⁵ is H.

5. The compound according to claim 1 or 2, wherein the compound is of Formula IB, and R⁵ is H.

6. The compound according to any one of claims 1 to 5, wherein Y is —NR^{Y1}—.

7. The compound according to any one of claims 1 to 5, wherein n^{A} is 1 or 2.

8. The compound according to any one of claims 1 to 7, wherein n^{A} is 1.

9. The compound according to any one of claims 1 to 7, wherein n^{A} is 2.

10. The compound according to claim 8 or 9, wherein R^{X1} and R^{X2} together with the carbon atom to which they are bound form a spiro-died C₃-C₅-cycloalkyl.

11. The compound according to claim 9, wherein any two vicinal R^{X1} and R^{X2} together with the carbon atoms to which they are bound form a C₃-C₅-cycloalkyl.

12. The compound according to claim 10 or 11, wherein the C₃-C₅-cycloalkyl is cyclopropyl.

13. The compound according to claim 8, wherein R^{X1} or R^{X2} is C₁-C₆-haloalkyl.

14. The compound according to claim 13, wherein R^{X1} is H.

15. The compound according to claim 8, 13, or 14, wherein R^{X1} is H and R^{X2} is —CF₃.

16. The compound according to any one of claims 1 to 5, wherein Y is —NR^{Y1}—, n^{A} is 1 or 2 and n^{B} is 2, and, R^{Y1} and one of R^{X1} and R^{X2}, together with the atoms to which they are bound, form a 3- to 6-membered heterocycloalkyl.

17. The compound according to claim 16, wherein the 3- to 6-membered heterocycloalkyl is piperazinyl.

18. The compound according to any one of claims 1 to 17, wherein R¹ and R² are independently selected from the group consisting of C₁-C₆-alkyl, C₁-C₆-haloalkyl, and —(C₁-C₆-alkyl)(C₃-C₈-cycloalkyl).

19. The compound according to any one of claims 1 to 18, wherein R¹ and R² are independently C₁-C₆-alkyl.

20. The compound according to any one of claims 1 to 19, wherein R¹ and R² are independently C₄-C₆-alkyl.

21. The compound according to any one of claims 1 to 19, wherein R¹ and R² are independently selected from iso-butyl and neo-pentyl.

22. The compound according to any one of claims 1 to 18, wherein R¹ and R² are independently —(C₁-C₆-alkyl)(C₃-C₈-cycloalkyl).

23. The compound according to any one of claims 1 to 22, wherein Z is —NR^{Z1}R^{Z2}.

24. the compound according to any one of claims 1 to 22, wherein Z is C₁-C₆-alkyl.

25. The compound according to any one of claims 1 to 22, wherein Z is C₃-C₅-cycloalkyl.

26. The compound according to any one of claims 1 to 25, wherein R³ is H.

27. The compound according to any one of claims 1 to 25, wherein R⁴ is H.

28. The compound according to claim 1 or 2, wherein:
the compound is of Formula IA;
X is CH;
Y is —NR^{Y1}—;
n^{A} is 1 or 2;
R^{X1} and R^{X2} are independently selected from the group consisting of H, C₁-C₆-alkyl, and C₁-C₆-haloalkyl;
R¹ is C₁-C₆-alkyl;
Z is —NR^{Z1}R^{Z2} or C₁-C₆-alkyl; and
R³ or R⁴ is H.

29. The compound according to claim 1, wherein:
the compound is of Formula IA;
X is CH or N;
Y is —NR^{Y1}—;
n^{A} is 1;
R^{X1} is H;
R^{X2} is C₁-C₆-alkyl or C₁-C₆-haloalkyl;
R¹ is C₁-C₆-alkyl;
Z is —C₃-C₅-cycloalkyl;
R³ is halo; and
R⁴ is optionally substituted C₆-C₁₀-aryl or 5- to 7-membered heteroaryl (wherein 1-4 heteroaryl members are independently selected from N, O, and S), and 3- to 6-membered heterocycloalkyl (wherein 1-4 ring members are independently selected from N, O, and S).

30. The compound according to claim 29, wherein:
R^{Y1} is H;
R^{X2} is selected from the group consisting of CH₃, CH₂F, CHF₂, and CF₃;
R¹ is —CH₂^{t}Bu; and
R⁴ is phenyl or pyridyl, wherein R⁴ is substituted with one or two substituents independently selected from the group consisting of F, Cl, CF₃, and CN.

31. A compound or pharmaceutically acceptable salt thereof according to claim 1 that is selected from the following table:

| | |
|---|---|
| 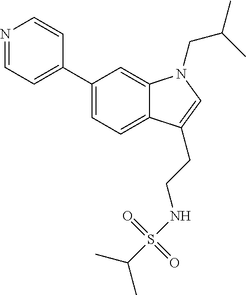 [SMILES: CC(C)Cn1cc(CCNS(=O)(=O)C(C)C)c2ccc(-c3ccncc3)cc21] | 1 |
| 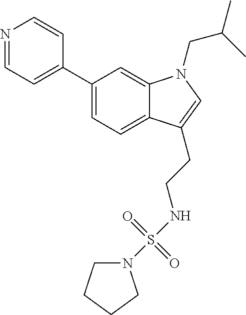 [SMILES: CC(C)Cn1cc(CCNS(=O)(=O)N2CCCC2)c2ccc(-c3ccncc3)cc21] | 2 |
| 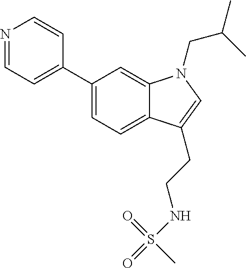 [SMILES: CC(C)Cn1cc(CCNS(C)(=O)=O)c2ccc(-c3ccncc3)cc21] | 3 |
| 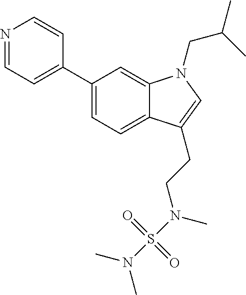 [SMILES: CC(C)Cn1cc(CCN(C)S(=O)(=O)N(C)C)c2ccc(-c3ccncc3)cc21] | 5 |
| 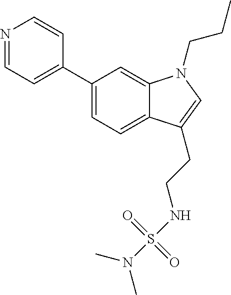 [SMILES: CCCn1cc(CCNS(=O)(=O)N(C)C)c2ccc(-c3ccncc3)cc21] | 6 |
| 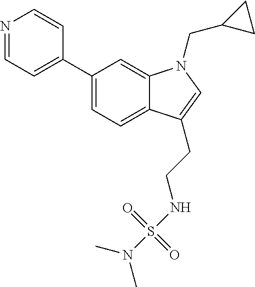 [SMILES: CN(C)S(=O)(=O)NCCc1cn(CC2CC2)c2cc(-c3ccncc3)ccc12] | 7 |
| 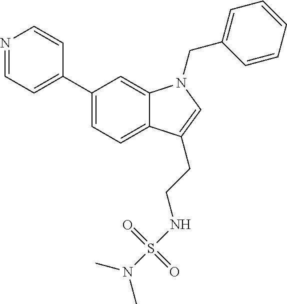 [SMILES: CN(C)S(=O)(=O)NCCc1cn(Cc2ccccc2)c2cc(-c3ccncc3)ccc12] | 8 |
| 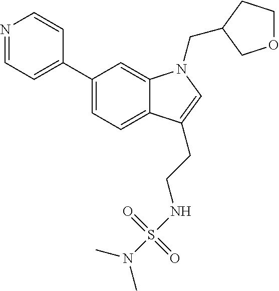 [SMILES: CN(C)S(=O)(=O)NCCc1cn(CC2CCOC2)c2cc(-c3ccncc3)ccc12] | 9 |
| 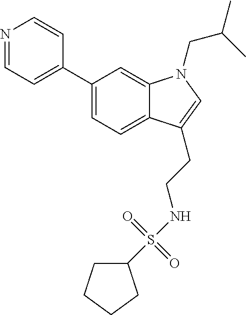 [SMILES: CC(C)Cn1cc(CCNS(=O)(=O)C2CCCC2)c2ccc(-c3ccncc3)cc21] | 11 |
| 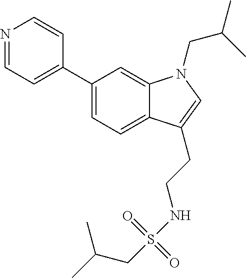 [SMILES: CC(C)Cn1cc(CCNS(=O)(=O)CC(C)C)c2ccc(-c3ccncc3)cc21] | 12 |
| 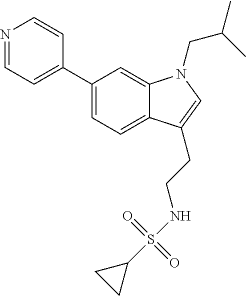 [SMILES: CC(C)Cn1cc(CCNS(=O)(=O)C2CC2)c2ccc(-c3ccncc3)cc21] | 13 |
| 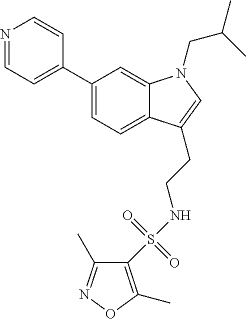 [SMILES: Cc1noc(C)c1S(=O)(=O)NCCc1cn(CC(C)C)c2cc(-c3ccncc3)ccc12] | 14 |
| 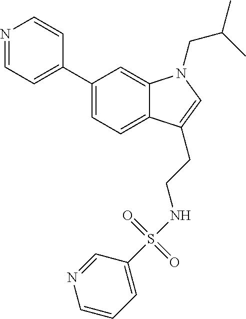 [SMILES: CC(C)Cn1cc(CCNS(=O)(=O)c2cccnc2)c2ccc(-c3ccncc3)cc21] | 15 |
| 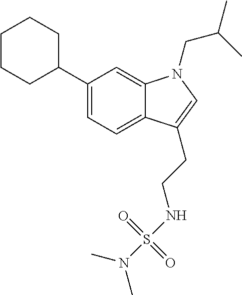 [SMILES: CC(C)Cn1cc(CCNS(=O)(=O)N(C)C)c2ccc(C3CCCCC3)cc21] | 16 |
| 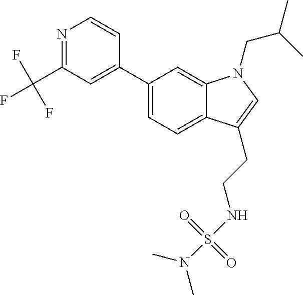 [SMILES: CC(C)Cn1cc(CCNS(=O)(=O)N(C)C)c2ccc(-c3ccnc(C(F)(F)F)c3)cc21] | 17 |
| 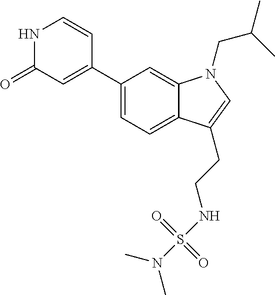 [SMILES: CC(C)Cn1cc(CCNS(=O)(=O)N(C)C)c2ccc(-c3cc[nH]c(=O)c3)cc21] | 18 |
| 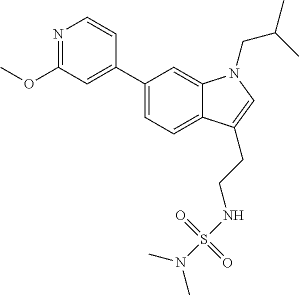 [SMILES: COc1cc(-c2ccc3c(CCNS(=O)(=O)N(C)C)cn(CC(C)C)c3c2)ccn1] | 19 |
| 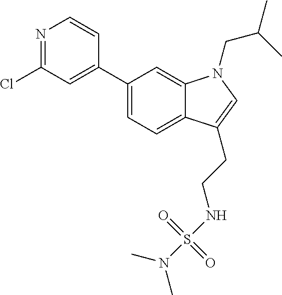 [SMILES: CC(C)Cn1cc(CCNS(=O)(=O)N(C)C)c2ccc(-c3ccnc(Cl)c3)cc21] | 20 |
| 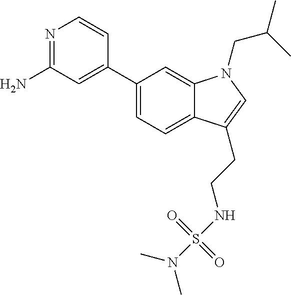 [SMILES: CC(C)Cn1cc(CCNS(=O)(=O)N(C)C)c2ccc(-c3ccnc(N)c3)cc21] | 21 |
| 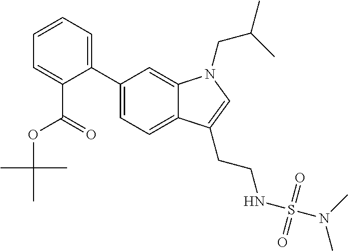 [SMILES: CC(C)Cn1cc(CCNS(=O)(=O)N(C)C)c2ccc(-c3ccccc3C(=O)OC(C)(C)C)cc21] | 22 |
| 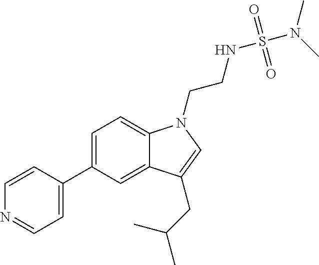 [SMILES: CC(C)Cc1cn(CCNS(=O)(=O)N(C)C)c2ccc(-c3ccncc3)cc12] | 23 |
| 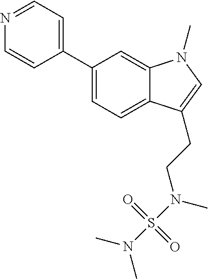 [SMILES: CN(C)S(=O)(=O)N(C)CCc1cn(C)c2cc(-c3ccncc3)ccc12] | 24 |
| 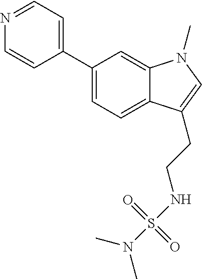 [SMILES: CN(C)S(=O)(=O)NCCc1cn(C)c2cc(-c3ccncc3)ccc12] | 25 |
| 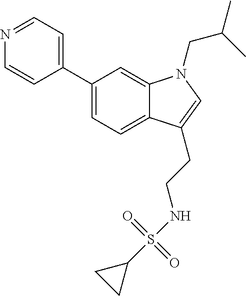 [SMILES: CC(C)Cn1cc(CCNS(=O)(=O)C2CC2)c2ccc(-c3ccncc3)cc21] | 26 |
| 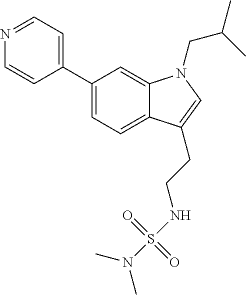 [SMILES: CC(C)Cn1cc(CCNS(=O)(=O)N(C)C)c2ccc(-c3ccncc3)cc21] | 27 |
| 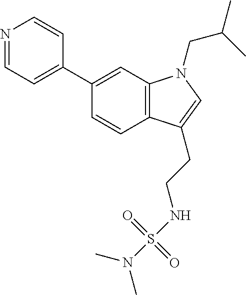 [SMILES: CC(C)Cn1cc(CCNS(=O)(=O)N(C)C)c2ccc(-c3ccncc3)cc21] | 28 |
| 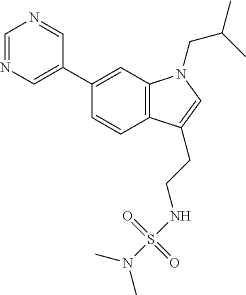 [SMILES: CC(C)Cn1cc(CCNS(=O)(=O)N(C)C)c2ccc(-c3cncnc3)cc21] | 29 |
| 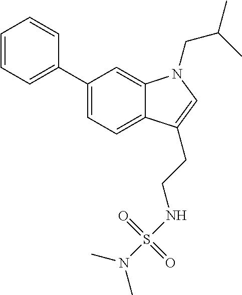 [SMILES: CC(C)Cn1cc(CCNS(=O)(=O)N(C)C)c2ccc(-c3ccccc3)cc21] | 30 |
| 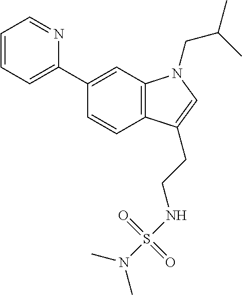 [SMILES: CC(C)Cn1cc(CCNS(=O)(=O)N(C)C)c2ccc(-c3ccccn3)cc21] | 31 |
| 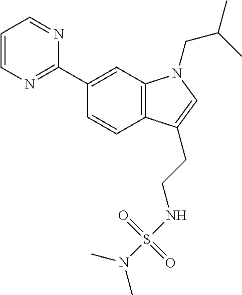 [SMILES: CC(C)Cn1cc(CCNS(=O)(=O)N(C)C)c2ccc(-c3ncccn3)cc21] | 32 |
| 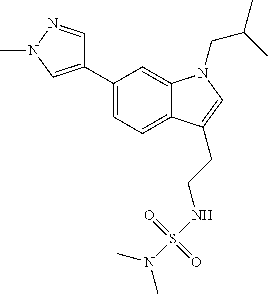 [SMILES: CC(C)Cn1cc(CCNS(=O)(=O)N(C)C)c2ccc(-c3cnn(C)c3)cc21] | 33 |
| 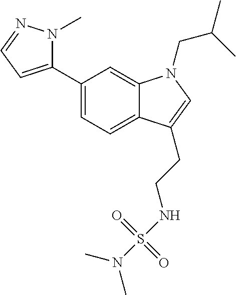 [SMILES: CC(C)Cn1cc(CCNS(=O)(=O)N(C)C)c2ccc(-c3ccnn3C)cc21] | 34 |
| 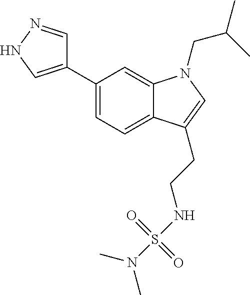 [SMILES: CC(C)Cn1cc(CCNS(=O)(=O)N(C)C)c2ccc(-c3cn[nH]c3)cc21] | 35 |
| 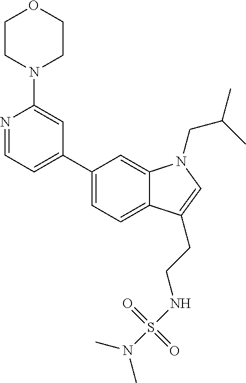 [SMILES: CC(C)Cn1cc(CCNS(=O)(=O)N(C)C)c2ccc(-c3ccnc(N4CCOCC4)c3)cc21] | 36 |
| 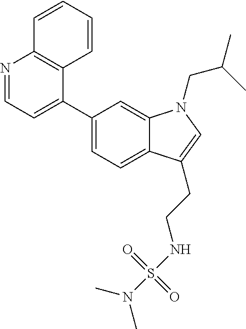 [SMILES: CC(C)Cn1cc(CCNS(=O)(=O)N(C)C)c2ccc(-c3ccnc4ccccc34)cc21] | 37 |
| 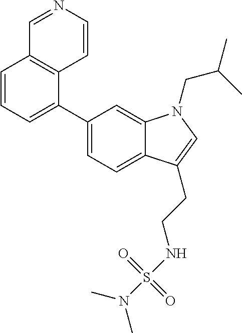 [SMILES: CC(C)Cn1cc(CCNS(=O)(=O)N(C)C)c2ccc(-c3cccc4cnccc34)cc21] | 38 |
| 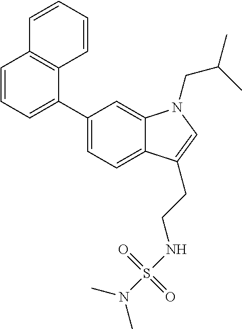 [SMILES: CC(C)Cn1cc(CCNS(=O)(=O)N(C)C)c2ccc(-c3cccc4ccccc34)cc21] | 39 |
| 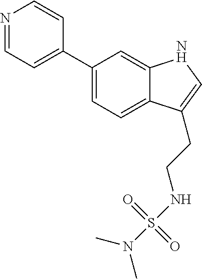 [SMILES: CN(C)S(=O)(=O)NCCc1c[nH]c2cc(-c3ccncc3)ccc12] | 40 |
| 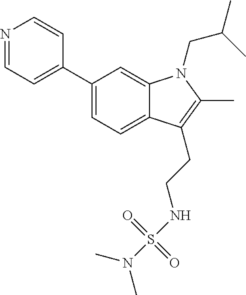 [SMILES: Cc1c(CCNS(=O)(=O)N(C)C)c2ccc(-c3ccncc3)cc2n1CC(C)C] | 41 |
| 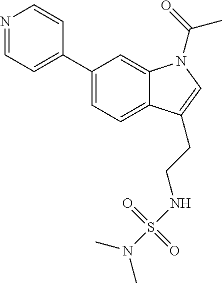 [SMILES: CC(=O)n1cc(CCNS(=O)(=O)N(C)C)c2ccc(-c3ccncc3)cc21] | 42 |
| 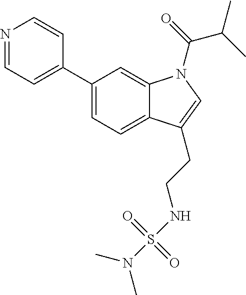 [SMILES: CC(C)C(=O)n1cc(CCNS(=O)(=O)N(C)C)c2ccc(-c3ccncc3)cc21] | 43 |
| 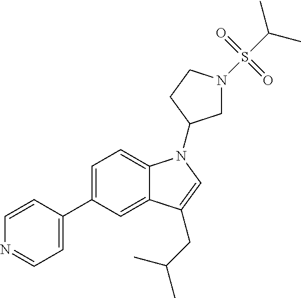 [SMILES: CC(C)Cc1cn(C2CCN(S(=O)(=O)C(C)C)C2)c2ccc(-c3ccncc3)cc12] | 44 |
| 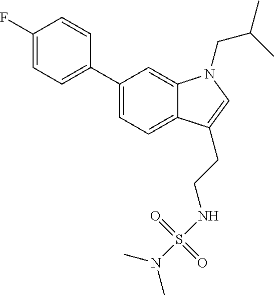 [SMILES: CC(C)Cn1cc(CCNS(=O)(=O)N(C)C)c2ccc(-c3ccc(F)cc3)cc21] | 45 |
| 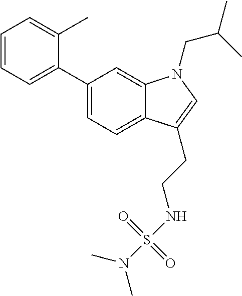 [SMILES: Cc1ccccc1-c1ccc2c(CCNS(=O)(=O)N(C)C)cn(CC(C)C)c2c1] | 46 |
| 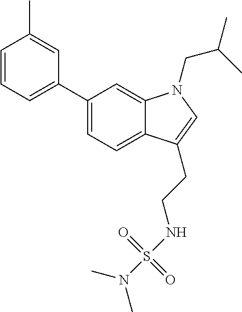 [SMILES: Cc1cccc(-c2ccc3c(CCNS(=O)(=O)N(C)C)cn(CC(C)C)c3c2)c1] | 47 |
| 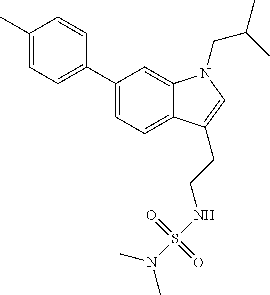 [SMILES: Cc1ccc(-c2ccc3c(CCNS(=O)(=O)N(C)C)cn(CC(C)C)c3c2)cc1] | 48 |
| 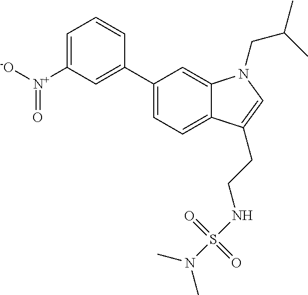 | 49 |
| 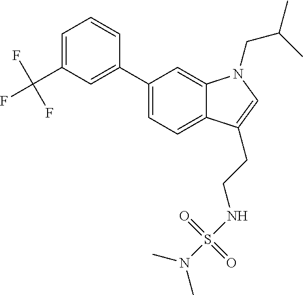 [SMILES: CC(C)Cn1cc(CCNS(=O)(=O)N(C)C)c2ccc(-c3cccc(C(F)(F)F)c3)cc21] | 50 |
| 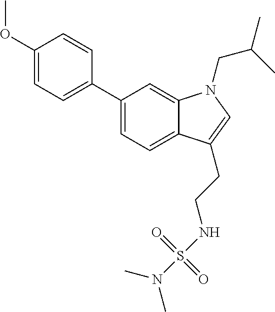 [SMILES: COc1ccc(-c2ccc3c(CCNS(=O)(=O)N(C)C)cn(CC(C)C)c3c2)cc1] | 51 |
| 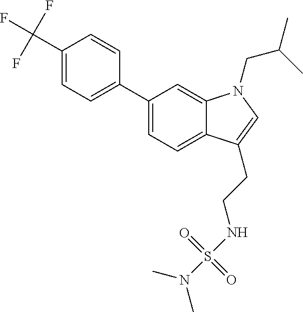 [SMILES: CC(C)Cn1cc(CCNS(=O)(=O)N(C)C)c2ccc(-c3ccc(C(F)(F)F)cc3)cc21] | 52 |
| 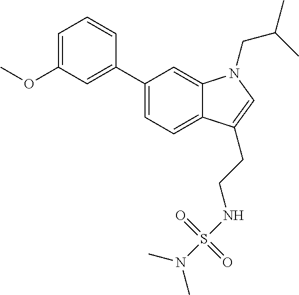 [SMILES: COc1cccc(-c2ccc3c(CCNS(=O)(=O)N(C)C)cn(CC(C)C)c3c2)c1] | 53 |
| 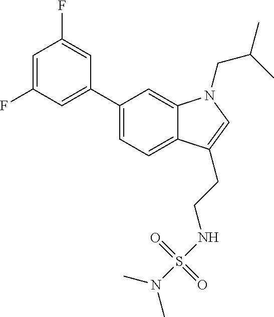 [SMILES: CC(C)Cn1cc(CCNS(=O)(=O)N(C)C)c2ccc(-c3cc(F)cc(F)c3)cc21] | 54 |
| 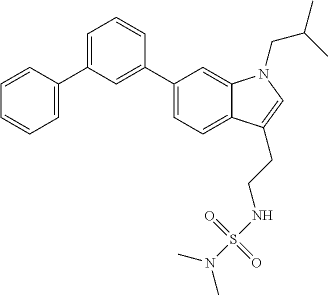 [SMILES: CC(C)Cn1cc(CCNS(=O)(=O)N(C)C)c2ccc(-c3cccc(-c4ccccc4)c3)cc21] | 55 |
| 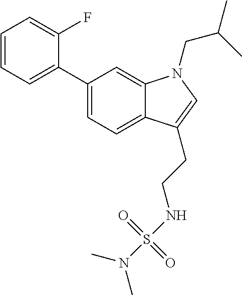 [SMILES: CC(C)Cn1cc(CCNS(=O)(=O)N(C)C)c2ccc(-c3ccccc3F)cc21] | 56 |
| 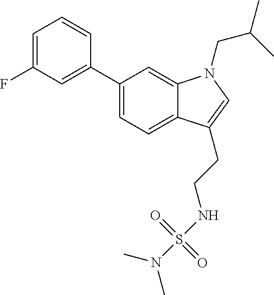 [SMILES: CC(C)Cn1cc(CCNS(=O)(=O)N(C)C)c2ccc(-c3cccc(F)c3)cc21] | 57 |
| 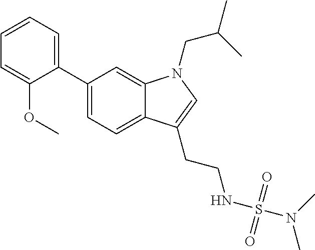 [SMILES: COc1ccccc1-c1ccc2c(CCNS(=O)(=O)N(C)C)cn(CC(C)C)c2c1] | 58 |
| 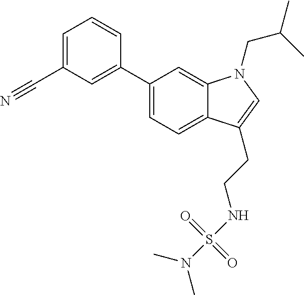 [SMILES: CC(C)Cn1cc(CCNS(=O)(=O)N(C)C)c2ccc(-c3cccc(C#N)c3)cc21] | 59 |
| 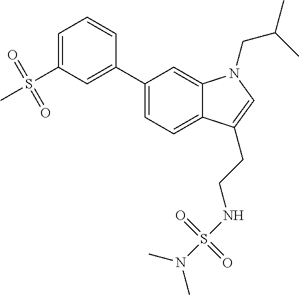 [SMILES: CC(C)Cn1cc(CCNS(=O)(=O)N(C)C)c2ccc(-c3cccc(S(C)(=O)=O)c3)cc21] | 60 |
| 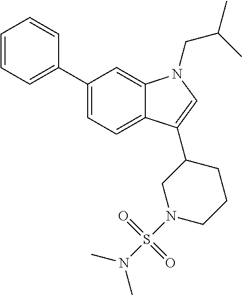 [SMILES: CC(C)Cn1cc(C2CCCN(S(=O)(=O)N(C)C)C2)c2ccc(-c3ccccc3)cc21] | 61 |
| 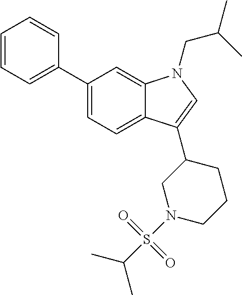 [SMILES: CC(C)Cn1cc(C2CCCN(S(=O)(=O)C(C)C)C2)c2ccc(-c3ccccc3)cc21] | 62 |
| 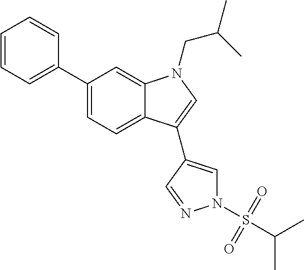 [SMILES: CC(C)Cn1cc(-c2cnn(S(=O)(=O)C(C)C)c2)c2ccc(-c3ccccc3)cc21] | 63 |
| 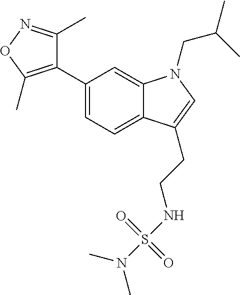 [SMILES: Cc1noc(C)c1-c1ccc2c(CCNS(=O)(=O)N(C)C)cn(CC(C)C)c2c1] | 64 |
| 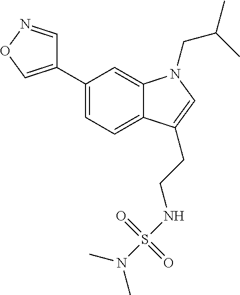 [SMILES: CC(C)Cn1cc(CCNS(=O)(=O)N(C)C)c2ccc(-c3cnoc3)cc21] | 65 |
| 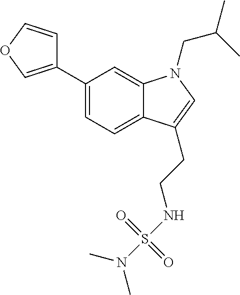 [SMILES: CC(C)Cn1cc(CCNS(=O)(=O)N(C)C)c2ccc(-c3ccoc3)cc21] | 66 |
| 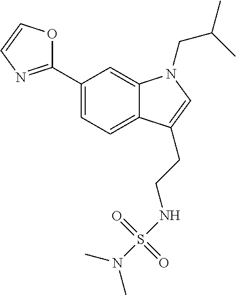 [SMILES: CC(C)Cn1cc(CCNS(=O)(=O)N(C)C)c2ccc(-c3ncco3)cc21] | 67 |
| 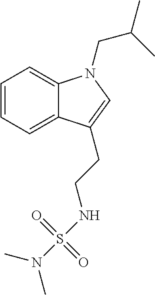 [SMILES: CC(C)Cn1cc(CCNS(=O)(=O)N(C)C)c2ccccc21] | 68 |
| 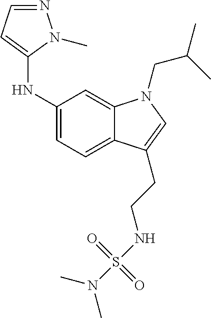 [SMILES: CC(C)Cn1cc(CCNS(=O)(=O)N(C)C)c2ccc(Nc3ccnn3C)cc21] | 69 |
| 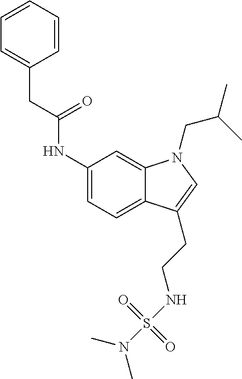 [SMILES: CC(C)Cn1cc(CCNS(=O)(=O)N(C)C)c2ccc(NC(=O)Cc3ccccc3)cc21] | 70 |
| 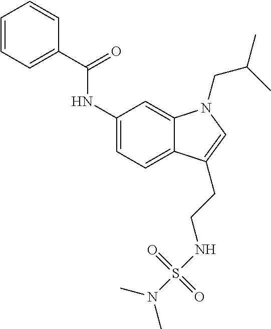 [SMILES: CC(C)Cn1cc(CCNS(=O)(=O)N(C)C)c2ccc(NC(=O)c3ccccc3)cc21] | 71 |
| 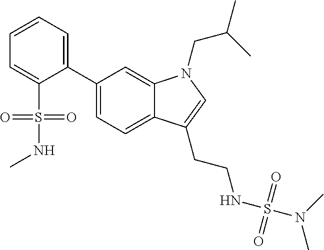 [SMILES: CNS(=O)(=O)c1ccccc1-c1ccc2c(CCNS(=O)(=O)N(C)C)cn(CC(C)C)c2c1] | 72 |
| 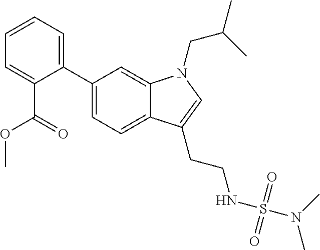 [SMILES: COC(=O)c1ccccc1-c1ccc2c(CCNS(=O)(=O)N(C)C)cn(CC(C)C)c2c1] | 73 |
| 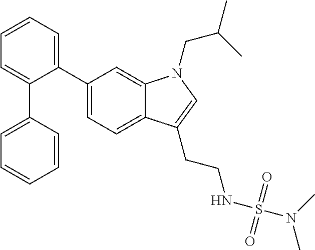 [SMILES: CC(C)Cn1cc(CCNS(=O)(=O)N(C)C)c2ccc(-c3ccccc3-c3ccccc3)cc21] | 74 |
| 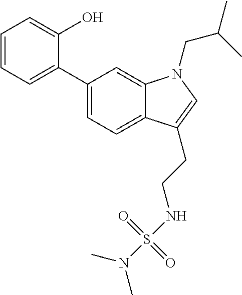 [SMILES: CC(C)Cn1cc(CCNS(=O)(=O)N(C)C)c2ccc(-c3ccccc3O)cc21] | 75 |
| 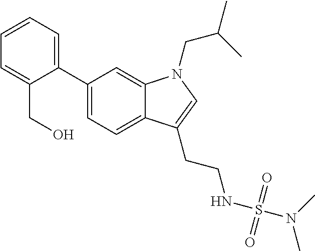 [SMILES: CC(C)Cn1cc(CCNS(=O)(=O)N(C)C)c2ccc(-c3ccccc3CO)cc21] | 76 |
| 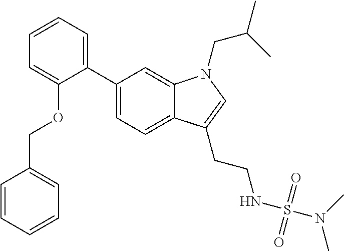 [SMILES: CC(C)Cn1cc(CCNS(=O)(=O)N(C)C)c2ccc(-c3ccccc3OCc3ccccc3)cc21] | 77 |
| 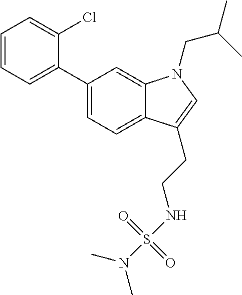 [SMILES: CC(C)Cn1cc(CCNS(=O)(=O)N(C)C)c2ccc(-c3ccccc3Cl)cc21] | 78 |
| 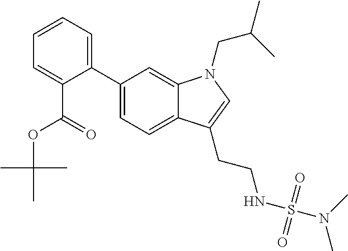 [SMILES: CC(C)Cn1cc(CCNS(=O)(=O)N(C)C)c2ccc(-c3ccccc3C(=O)OC(C)(C)C)cc21] | 79 |
| 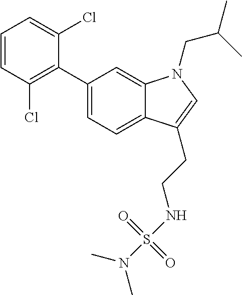 [SMILES: CC(C)Cn1cc(CCNS(=O)(=O)N(C)C)c2ccc(-c3c(Cl)cccc3Cl)cc21] | 80 |
| 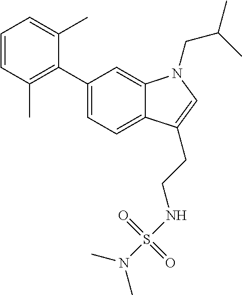 [SMILES: Cc1cccc(C)c1-c1ccc2c(CCNS(=O)(=O)N(C)C)cn(CC(C)C)c2c1] | 81 |
| 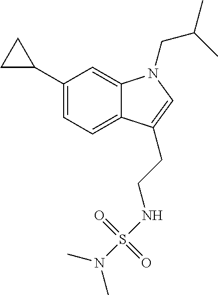 [SMILES: CC(C)Cn1cc(CCNS(=O)(=O)N(C)C)c2ccc(C3CC3)cc21] | 82 |
| 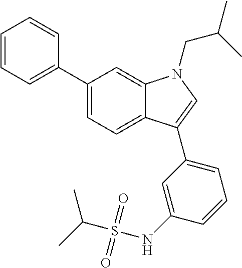 [SMILES: CC(C)Cn1cc(-c2cccc(NS(=O)(=O)C(C)C)c2)c2ccc(-c3ccccc3)cc21] | 83 |
| 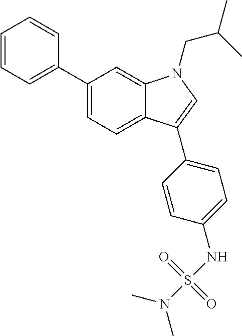 [SMILES: CC(C)Cn1cc(-c2ccc(NS(=O)(=O)N(C)C)cc2)c2ccc(-c3ccccc3)cc21] | 84 |
| 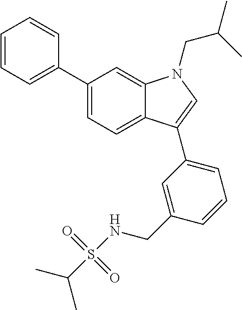 [SMILES: CC(C)Cn1cc(-c2cccc(CNS(=O)(=O)C(C)C)c2)c2ccc(-c3ccccc3)cc21] | 86 |
| 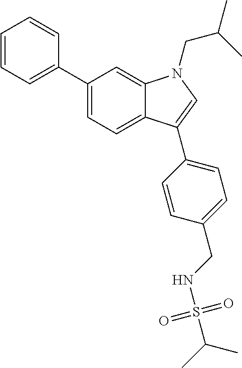 [SMILES: CC(C)Cn1cc(-c2ccc(CNS(=O)(=O)C(C)C)cc2)c2ccc(-c3ccccc3)cc21] | 87 |
| 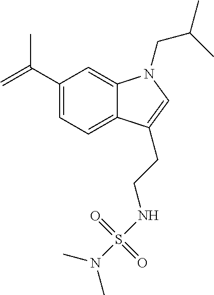 [SMILES: C=C(C)c1ccc2c(CCNS(=O)(=O)N(C)C)cn(CC(C)C)c2c1] | 88 |
| 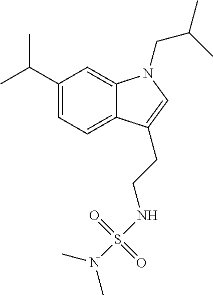 [SMILES: CC(C)Cn1cc(CCNS(=O)(=O)N(C)C)c2ccc(C(C)C)cc21] | 91 |
| 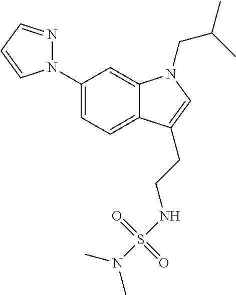 [SMILES: CC(C)Cn1cc(CCNS(=O)(=O)N(C)C)c2ccc(-n3cccn3)cc21] | 92 |
| 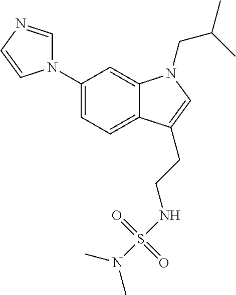 [SMILES: CC(C)Cn1cc(CCNS(=O)(=O)N(C)C)c2ccc(-n3ccnc3)cc21] | 93 |
| 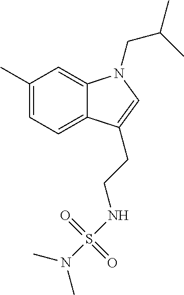 [SMILES: Cc1ccc2c(CCNS(=O)(=O)N(C)C)cn(CC(C)C)c2c1] | 95 |
| 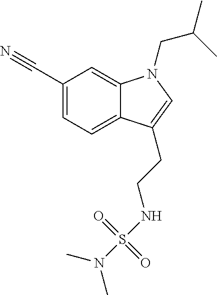 [SMILES: CC(C)Cn1cc(CCNS(=O)(=O)N(C)C)c2ccc(C#N)cc21] | 96 |
| 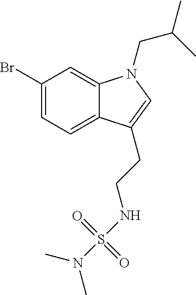 [SMILES: CC(C)Cn1cc(CCNS(=O)(=O)N(C)C)c2ccc(Br)cc21] | 97 |
| 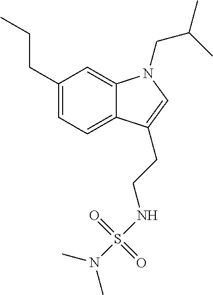 [SMILES: CCCc1ccc2c(CCNS(=O)(=O)N(C)C)cn(CC(C)C)c2c1] | 98 |
| 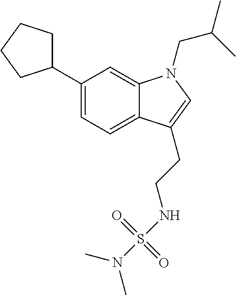 [SMILES: CC(C)Cn1cc(CCNS(=O)(=O)N(C)C)c2ccc(C3CCCC3)cc21] | 99 |
| 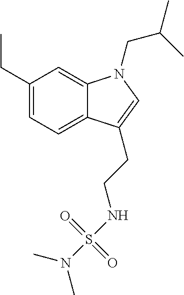 [SMILES: CCc1ccc2c(CCNS(=O)(=O)N(C)C)cn(CC(C)C)c2c1] | 100 |
| 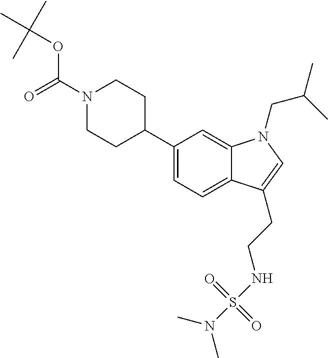 [SMILES: CC(C)Cn1cc(CCNS(=O)(=O)N(C)C)c2ccc(C3CCN(C(=O)OC(C)(C)C)CC3)cc21] | 101 |
| 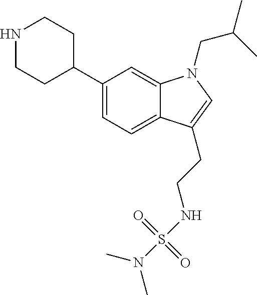 [SMILES: CC(C)Cn1cc(CCNS(=O)(=O)N(C)C)c2ccc(C3CCNCC3)cc21] | 102 |
| 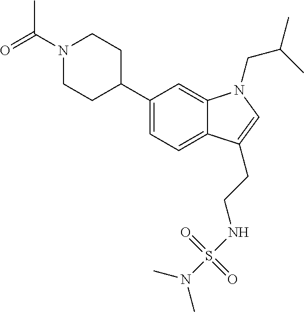 [SMILES: CC(=O)N1CCC(c2ccc3c(CCNS(=O)(=O)N(C)C)cn(CC(C)C)c3c2)CC1] | 103 |
| 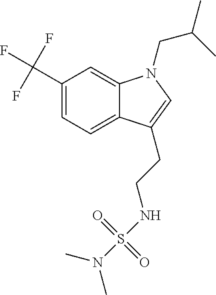 [SMILES: CC(C)Cn1cc(CCNS(=O)(=O)N(C)C)c2ccc(C(F)(F)F)cc21] | 105 |
| 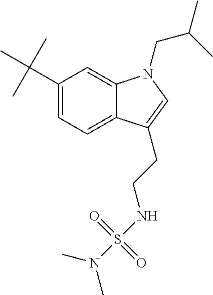 [SMILES: CC(C)Cn1cc(CCNS(=O)(=O)N(C)C)c2ccc(C(C)(C)C)cc21] | 106 |
| 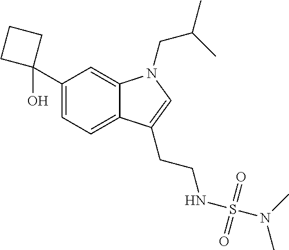 [SMILES: CC(C)Cn1cc(CCNS(=O)(=O)N(C)C)c2ccc(C3(O)CCC3)cc21] | 107 |
| 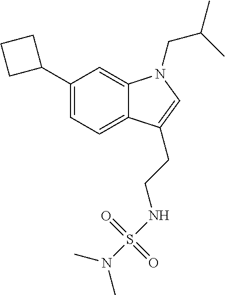 [SMILES: CC(C)Cn1cc(CCNS(=O)(=O)N(C)C)c2ccc(C3CCC3)cc21] | 108 |
| 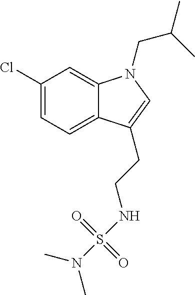 [SMILES: CC(C)Cn1cc(CCNS(=O)(=O)N(C)C)c2ccc(Cl)cc21] | 109 |
| 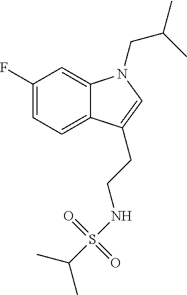 [SMILES: CC(C)Cn1cc(CCNS(=O)(=O)C(C)C)c2ccc(F)cc21] | 110 |
| 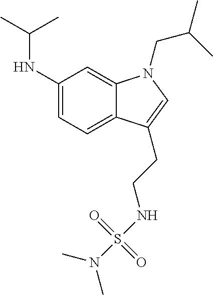 [SMILES: CC(C)Cn1cc(CCNS(=O)(=O)N(C)C)c2ccc(NC(C)C)cc21] | 111 |
| 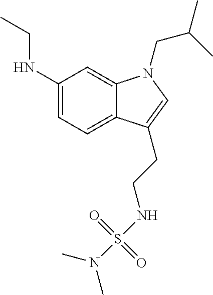 [SMILES: CCNc1ccc2c(CCNS(=O)(=O)N(C)C)cn(CC(C)C)c2c1] | 112 |
| 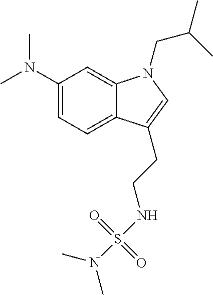 [SMILES: CC(C)Cn1cc(CCNS(=O)(=O)N(C)C)c2ccc(N(C)C)cc21] | 113 |
| 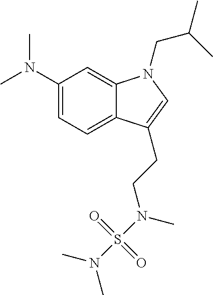 [SMILES: CC(C)Cn1cc(CCN(C)S(=O)(=O)N(C)C)c2ccc(N(C)C)cc21] | 114 |
| 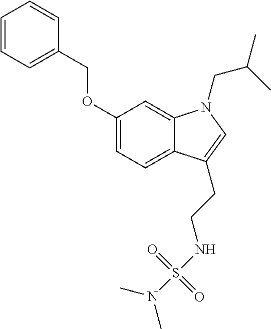 [SMILES: CC(C)Cn1cc(CCNS(=O)(=O)N(C)C)c2ccc(OCc3ccccc3)cc21] | 115 |
| 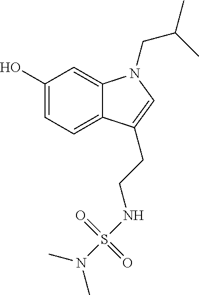 [SMILES: CC(C)Cn1cc(CCNS(=O)(=O)N(C)C)c2ccc(O)cc21] | 116 |
| 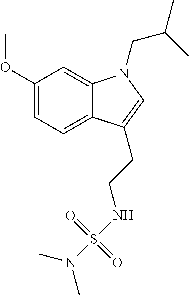 [SMILES: COc1ccc2c(CCNS(=O)(=O)N(C)C)cn(CC(C)C)c2c1] | 117 |
| 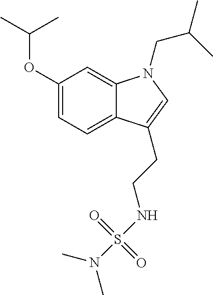 [SMILES: CC(C)Cn1cc(CCNS(=O)(=O)N(C)C)c2ccc(OC(C)C)cc21] | 118 |
| 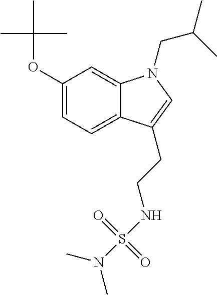 [SMILES: CC(C)Cn1cc(CCNS(=O)(=O)N(C)C)c2ccc(OC(C)(C)C)cc21] | 119 |
| 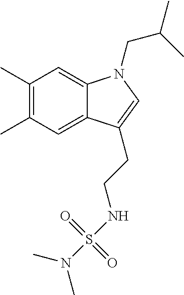 [SMILES: Cc1cc2c(CCNS(=O)(=O)N(C)C)cn(CC(C)C)c2cc1C] | 120 |
| 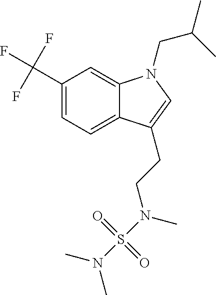 [SMILES: CC(C)Cn1cc(CCN(C)S(=O)(=O)N(C)C)c2ccc(C(F)(F)F)cc21] | 121 |
| 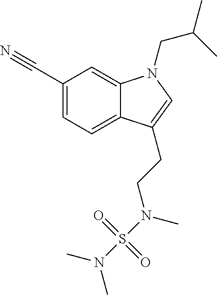 [SMILES: CC(C)Cn1cc(CCN(C)S(=O)(=O)N(C)C)c2ccc(C#N)cc21] | 122 |
| 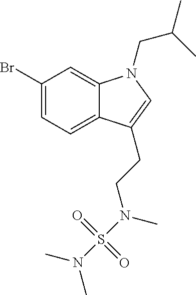 [SMILES: CC(C)Cn1cc(CCN(C)S(=O)(=O)N(C)C)c2ccc(Br)cc21] | 123 |
| 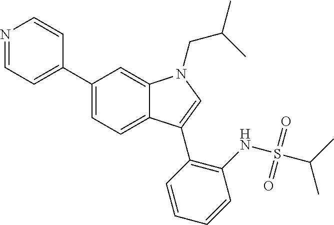 [SMILES: CC(C)Cn1cc(-c2ccccc2NS(=O)(=O)C(C)C)c2ccc(-c3ccncc3)cc21] | 124 |
| 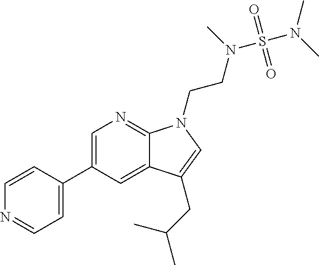 [SMILES: CC(C)Cc1cn(CCN(C)S(=O)(=O)N(C)C)c2ncc(-c3ccncc3)cc12] | 125 |
| 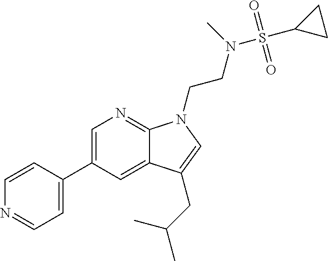 [SMILES: CC(C)Cc1cn(CCN(C)S(=O)(=O)C2CC2)c2ncc(-c3ccncc3)cc12] | 126 |
| 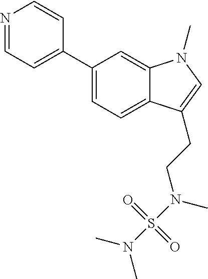 [SMILES: CN(C)S(=O)(=O)N(C)CCc1cn(C)c2cc(-c3ccncc3)ccc12] | 127 |
| 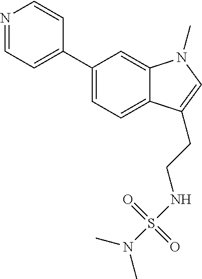 [SMILES: CN(C)S(=O)(=O)NCCc1cn(C)c2cc(-c3ccncc3)ccc12] | 128 |
| 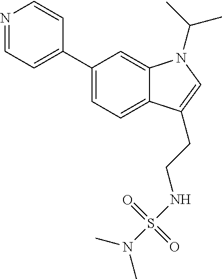 [SMILES: CC(C)n1cc(CCNS(=O)(=O)N(C)C)c2ccc(-c3ccncc3)cc21] | 129 |
| 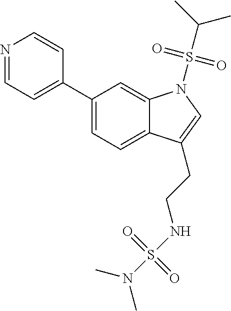 [SMILES: CC(C)S(=O)(=O)n1cc(CCNS(=O)(=O)N(C)C)c2ccc(-c3ccncc3)cc21] | 130 |
| 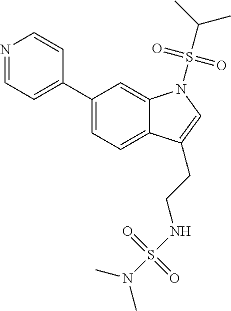 [SMILES: CC(C)S(=O)(=O)n1cc(CCNS(=O)(=O)N(C)C)c2ccc(-c3ccncc3)cc21] | 131 |
| 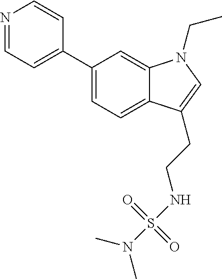 [SMILES: CCn1cc(CCNS(=O)(=O)N(C)C)c2ccc(-c3ccncc3)cc21] | 132 |
| 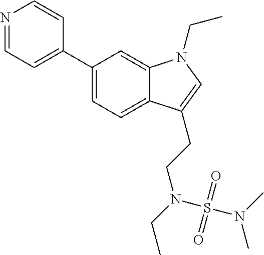 [SMILES: CCN(CCc1cn(CC)c2cc(-c3ccncc3)ccc12)S(=O)(=O)N(C)C] | 133 |
| 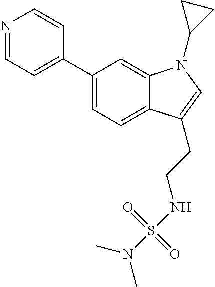 [SMILES: CN(C)S(=O)(=O)NCCc1cn(C2CC2)c2cc(-c3ccncc3)ccc12] | 134 |
| 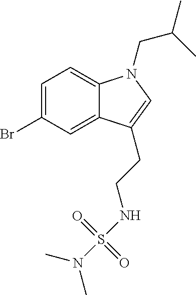 [SMILES: CC(C)Cn1cc(CCNS(=O)(=O)N(C)C)c2cc(Br)ccc21] | 135 |
| 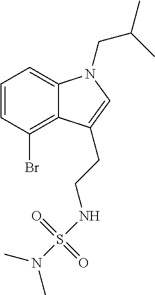 [SMILES: CC(C)Cn1cc(CCNS(=O)(=O)N(C)C)c2c(Br)cccc21] | 136 |
| 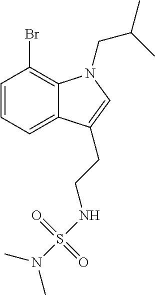 [SMILES: CC(C)Cn1cc(CCNS(=O)(=O)N(C)C)c2cccc(Br)c21] | 137 |
| 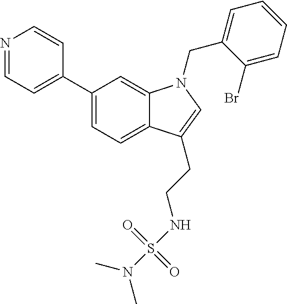 [SMILES: CN(C)S(=O)(=O)NCCc1cn(Cc2ccccc2Br)c2cc(-c3ccncc3)ccc12] | 140 |
| 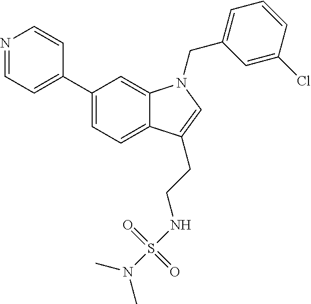 [SMILES: CN(C)S(=O)(=O)NCCc1cn(Cc2cccc(Cl)c2)c2cc(-c3ccncc3)ccc12] | 141 |
| 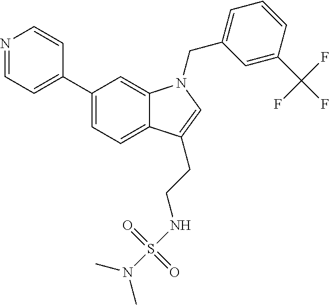 [SMILES: CN(C)S(=O)(=O)NCCc1cn(Cc2cccc(C(F)(F)F)c2)c2cc(-c3ccncc3)ccc12] | 142 |
| 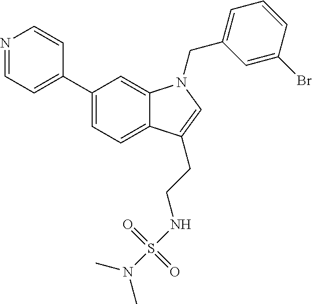 [SMILES: CN(C)S(=O)(=O)NCCc1cn(Cc2cccc(Br)c2)c2cc(-c3ccncc3)ccc12] | 143 |
| 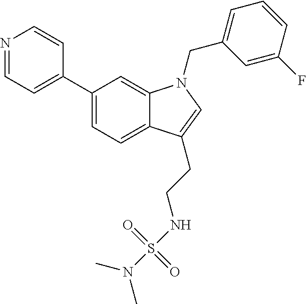 [SMILES: CN(C)S(=O)(=O)NCCc1cn(Cc2cccc(F)c2)c2cc(-c3ccncc3)ccc12] | 144 |
| 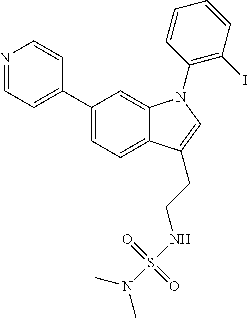 [SMILES: CN(C)S(=O)(=O)NCCc1cn(-c2ccccc2I)c2cc(-c3ccncc3)ccc12] | 145 |
| 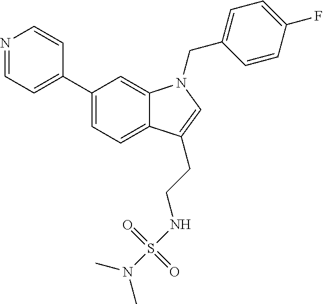 [SMILES: CN(C)S(=O)(=O)NCCc1cn(Cc2ccc(F)cc2)c2cc(-c3ccncc3)ccc12] | 146 |
| 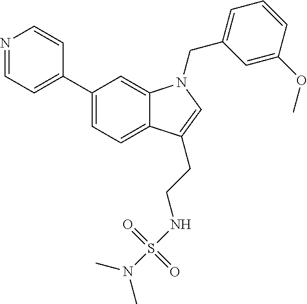 [SMILES: COc1cccc(Cn2cc(CCNS(=O)(=O)N(C)C)c3ccc(-c4ccncc4)cc32)c1] | 147 |
| 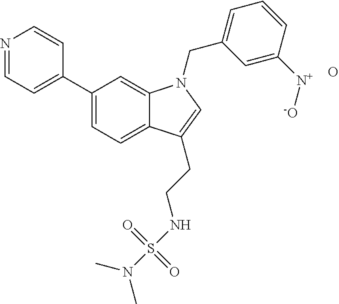 | 148 |
| 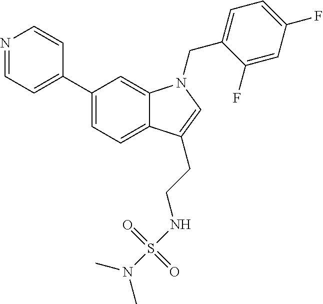 [SMILES: CN(C)S(=O)(=O)NCCc1cn(Cc2ccc(F)cc2F)c2cc(-c3ccncc3)ccc12] | 149 |
| 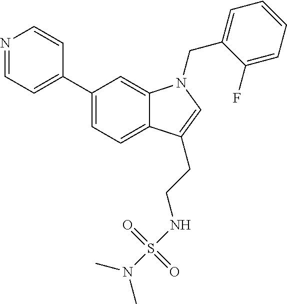 [SMILES: CN(C)S(=O)(=O)NCCc1cn(Cc2ccccc2F)c2cc(-c3ccncc3)ccc12] | 150 |
| 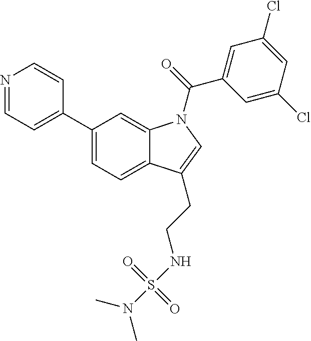 [SMILES: CN(C)S(=O)(=O)NCCc1cn(C(=O)c2cc(Cl)cc(Cl)c2)c2cc(-c3ccncc3)ccc12] | 151 |
| 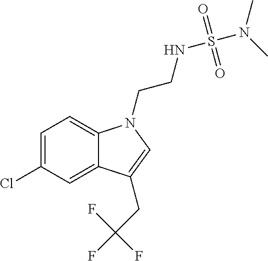 [SMILES: CN(C)S(=O)(=O)NCCn1cc(CC(F)(F)F)c2cc(Cl)ccc21] | 152 |
| 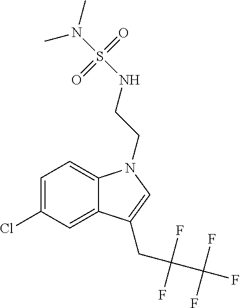 [SMILES: CN(C)S(=O)(=O)NCCn1cc(CC(F)(F)C(F)(F)F)c2cc(Cl)ccc21] | 153 |
| 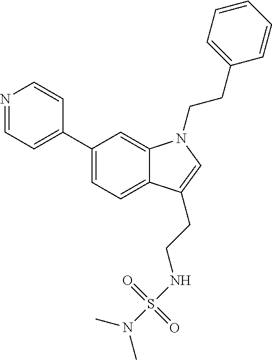 [SMILES: CN(C)S(=O)(=O)NCCc1cn(CCc2ccccc2)c2cc(-c3ccncc3)ccc12] | 154 |
| 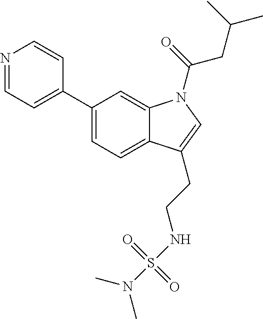 [SMILES: CC(C)CC(=O)n1cc(CCNS(=O)(=O)N(C)C)c2ccc(-c3ccncc3)cc21] | 155 |
| 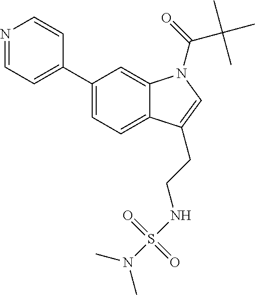 [SMILES: CN(C)S(=O)(=O)NCCc1cn(C(=O)C(C)(C)C)c2cc(-c3ccncc3)ccc12] | 156 |
| 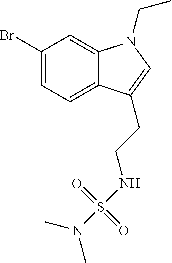 [SMILES: CCn1cc(CCNS(=O)(=O)N(C)C)c2ccc(Br)cc21] | 157 |
| 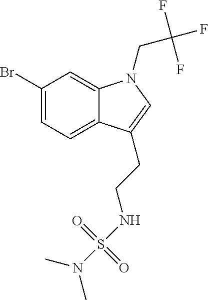 [SMILES: CN(C)S(=O)(=O)NCCc1cn(CC(F)(F)F)c2cc(Br)ccc12] | 158 |
| 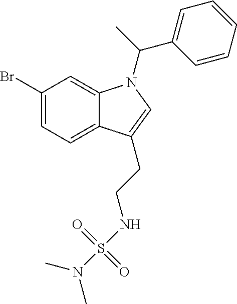 [SMILES: CC(c1ccccc1)n1cc(CCNS(=O)(=O)N(C)C)c2ccc(Br)cc21] | 159 |
| 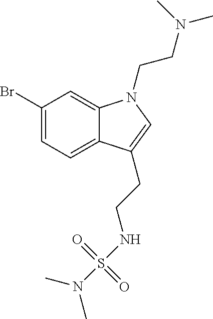 [SMILES: CN(C)CCn1cc(CCNS(=O)(=O)N(C)C)c2ccc(Br)cc21] | 160 |
| 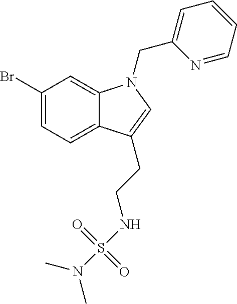 [SMILES: CN(C)S(=O)(=O)NCCc1cn(Cc2ccccn2)c2cc(Br)ccc12] | 161 |
| 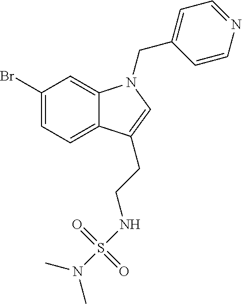 [SMILES: CN(C)S(=O)(=O)NCCc1cn(Cc2ccncc2)c2cc(Br)ccc12] | 162 |
| 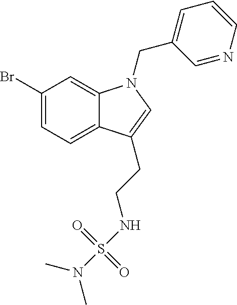 [SMILES: CN(C)S(=O)(=O)NCCc1cn(Cc2cccnc2)c2cc(Br)ccc12] | 163 |
| 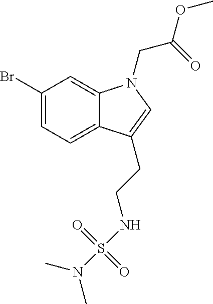 [SMILES: COC(=O)Cn1cc(CCNS(=O)(=O)N(C)C)c2ccc(Br)cc21] | 164 |
| 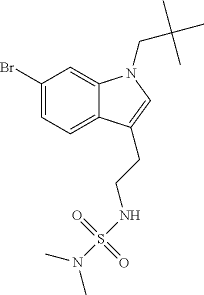 [SMILES: CN(C)S(=O)(=O)NCCc1cn(CC(C)(C)C)c2cc(Br)ccc12] | 165 |
| 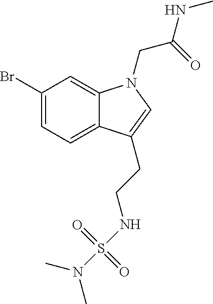 [SMILES: CNC(=O)Cn1cc(CCNS(=O)(=O)N(C)C)c2ccc(Br)cc21] | 166 |
| 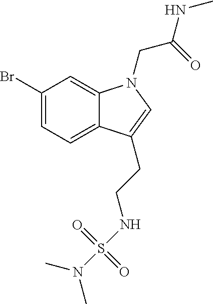 [SMILES: CNC(=O)Cn1cc(CCNS(=O)(=O)N(C)C)c2ccc(Br)cc21] | 167 |
| 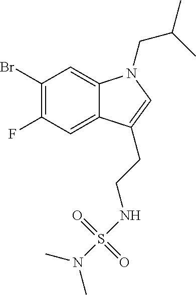 [SMILES: CC(C)Cn1cc(CCNS(=O)(=O)N(C)C)c2cc(F)c(Br)cc21] | 168 |
| 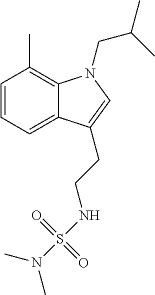 [SMILES: Cc1cccc2c(CCNS(=O)(=O)N(C)C)cn(CC(C)C)c12] | 169 |
| 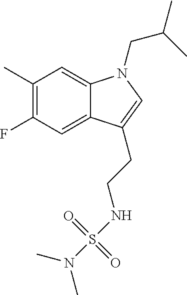 [SMILES: Cc1cc2c(cc1F)c(CCNS(=O)(=O)N(C)C)cn2CC(C)C] | 170 |
| 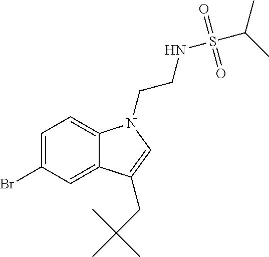 [SMILES: CC(C)S(=O)(=O)NCCn1cc(CC(C)(C)C)c2cc(Br)ccc21] | 171 |
| 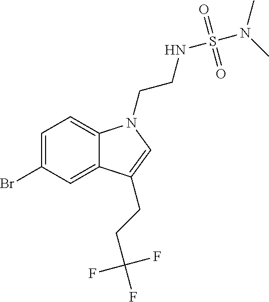 [SMILES: CN(C)S(=O)(=O)NCCn1cc(CCC(F)(F)F)c2cc(Br)ccc21] | 172 |
| 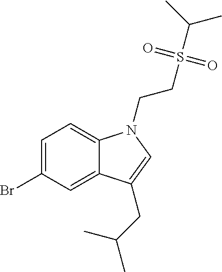 [SMILES: CC(C)Cc1cn(CCS(=O)(=O)C(C)C)c2ccc(Br)cc12] | 173 |
| 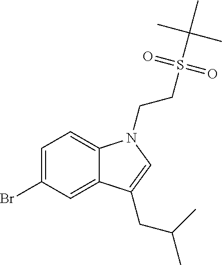 [SMILES: CC(C)Cc1cn(CCS(=O)(=O)C(C)(C)C)c2ccc(Br)cc12] | 174 |
| 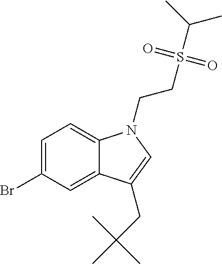 [SMILES: CC(C)S(=O)(=O)CCn1cc(CC(C)(C)C)c2cc(Br)ccc21] | 175 |
| 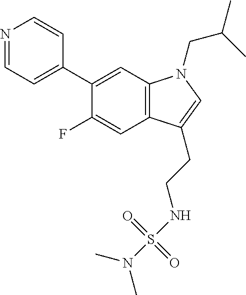 [SMILES: CC(C)Cn1cc(CCNS(=O)(=O)N(C)C)c2cc(F)c(-c3ccncc3)cc21] | 176 |
| 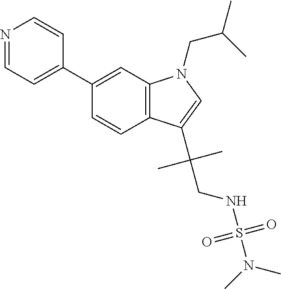 [SMILES: CC(C)Cn1cc(C(C)(C)CNS(=O)(=O)N(C)C)c2ccc(-c3ccncc3)cc21] | 177 |
| 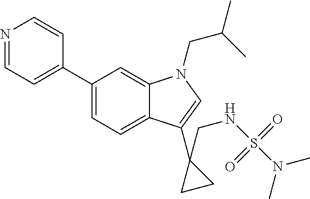 [SMILES: CC(C)Cn1cc(C2(CNS(=O)(=O)N(C)C)CC2)c2ccc(-c3ccncc3)cc21] | 178 |
| 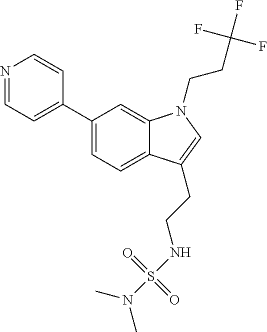 [SMILES: CN(C)S(=O)(=O)NCCc1cn(CCC(F)(F)F)c2cc(-c3ccncc3)ccc12] | 179 |
| 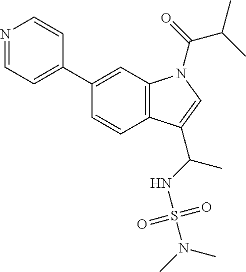 [SMILES: CC(C)C(=O)n1cc(C(C)NS(=O)(=O)N(C)C)c2ccc(-c3ccncc3)cc21] | 180 |
| 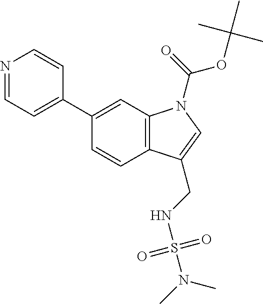 [SMILES: CN(C)S(=O)(=O)NCc1cn(C(=O)OC(C)(C)C)c2cc(-c3ccncc3)ccc12] | 181 |
| 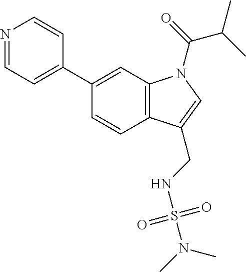 [SMILES: CC(C)C(=O)n1cc(CNS(=O)(=O)N(C)C)c2ccc(-c3ccncc3)cc21] | 182 |
| 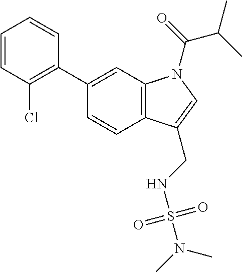 [SMILES: CC(C)C(=O)n1cc(CNS(=O)(=O)N(C)C)c2ccc(-c3ccccc3Cl)cc21] | 183 |
| 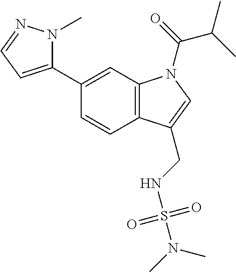 [SMILES: CC(C)C(=O)n1cc(CNS(=O)(=O)N(C)C)c2ccc(-c3ccnn3C)cc21] | 184 |
| 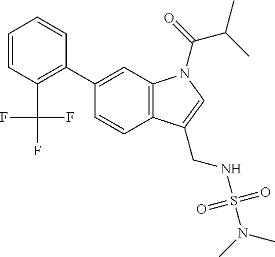 [SMILES: CC(C)C(=O)n1cc(CNS(=O)(=O)N(C)C)c2ccc(-c3ccccc3C(F)(F)F)cc21] | 185 |
| 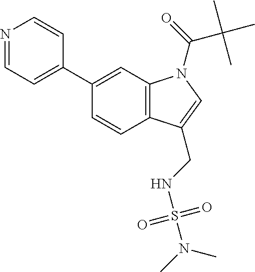 [SMILES: CN(C)S(=O)(=O)NCc1cn(C(=O)C(C)(C)C)c2cc(-c3ccncc3)ccc12] | 186 |
| 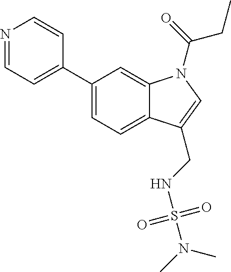 [SMILES: CCC(=O)n1cc(CNS(=O)(=O)N(C)C)c2ccc(-c3ccncc3)cc21] | 187 |
| 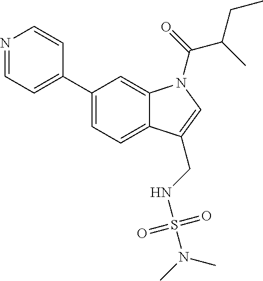 [SMILES: CCC(C)C(=O)n1cc(CNS(=O)(=O)N(C)C)c2ccc(-c3ccncc3)cc21] | 188 |
| 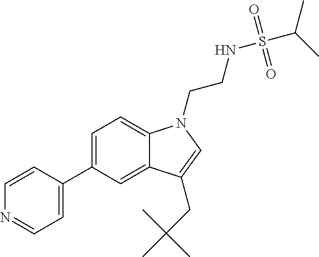 [SMILES: CC(C)S(=O)(=O)NCCn1cc(CC(C)(C)C)c2cc(-c3ccncc3)ccc21] | 189 |
| 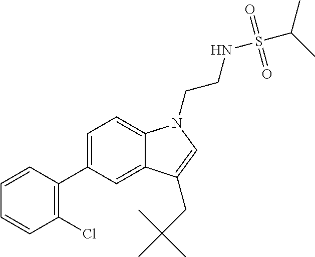 [SMILES: CC(C)S(=O)(=O)NCCn1cc(CC(C)(C)C)c2cc(-c3ccccc3Cl)ccc21] | 190 |
| 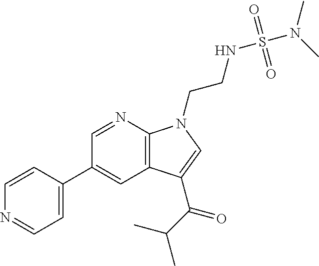 [SMILES: CC(C)C(=O)c1cn(CCNS(=O)(=O)N(C)C)c2ncc(-c3ccncc3)cc12] | 191 |
| 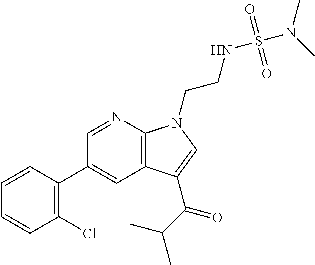 [SMILES: CC(C)C(=O)c1cn(CCNS(=O)(=O)N(C)C)c2ncc(-c3ccccc3Cl)cc12] | 192 |
| 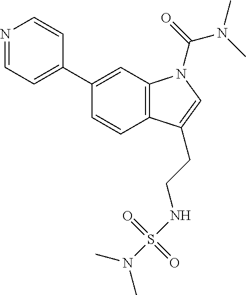 [SMILES: CN(C)C(=O)n1cc(CCNS(=O)(=O)N(C)C)c2ccc(-c3ccncc3)cc21] | 194 |
| 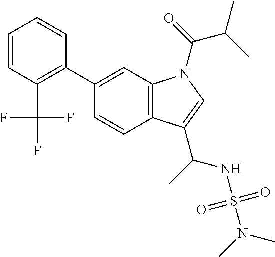 [SMILES: CC(C)C(=O)n1cc(C(C)NS(=O)(=O)N(C)C)c2ccc(-c3ccccc3C(F)(F)F)cc21] | 195 |
| 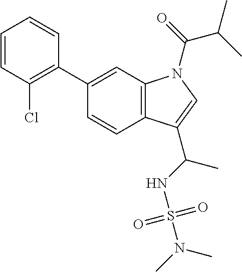 [SMILES: CC(C)C(=O)n1cc(C(C)NS(=O)(=O)N(C)C)c2ccc(-c3ccccc3Cl)cc21] | 196 |
| 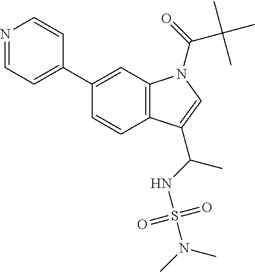 [SMILES: CC(NS(=O)(=O)N(C)C)c1cn(C(=O)C(C)(C)C)c2cc(-c3ccncc3)ccc12] | 197 |
| 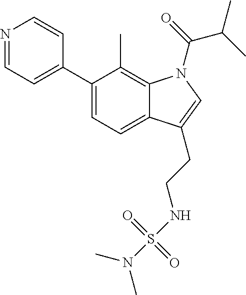 [SMILES: Cc1c(-c2ccncc2)ccc2c(CCNS(=O)(=O)N(C)C)cn(C(=O)C(C)C)c12] | 199 |
| 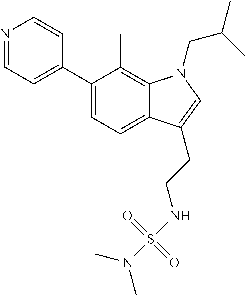 [SMILES: Cc1c(-c2ccncc2)ccc2c(CCNS(=O)(=O)N(C)C)cn(CC(C)C)c12] | 200 |
| 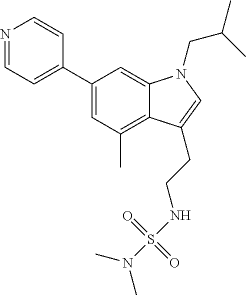 [SMILES: Cc1cc(-c2ccncc2)cc2c1c(CCNS(=O)(=O)N(C)C)cn2CC(C)C] | 202 |
| 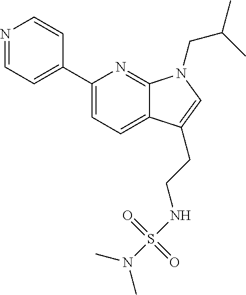 [SMILES: CC(C)Cn1cc(CCNS(=O)(=O)N(C)C)c2ccc(-c3ccncc3)nc21] | 203 |
| 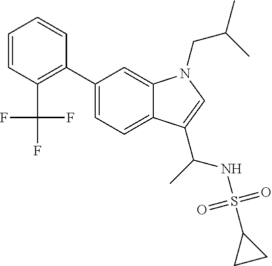 [SMILES: CC(C)Cn1cc(C(C)NS(=O)(=O)C2CC2)c2ccc(-c3ccccc3C(F)(F)F)cc21] | 204 |
| 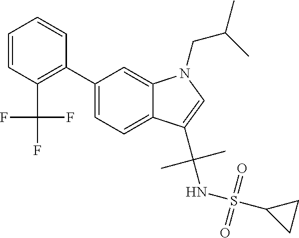 [SMILES: CC(C)Cn1cc(C(C)(C)NS(=O)(=O)C2CC2)c2ccc(-c3ccccc3C(F)(F)F)cc21] | 205 |
| 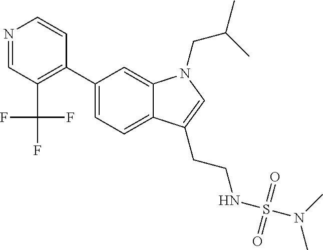 [SMILES: CC(C)Cn1cc(CCNS(=O)(=O)N(C)C)c2ccc(-c3ccncc3C(F)(F)F)cc21] | 206 |
| 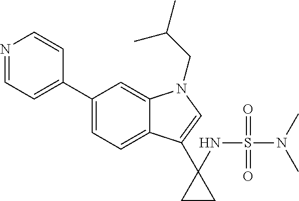 [SMILES: CC(C)Cn1cc(C2(NS(=O)(=O)N(C)C)CC2)c2ccc(-c3ccncc3)cc21] | 207 |
| 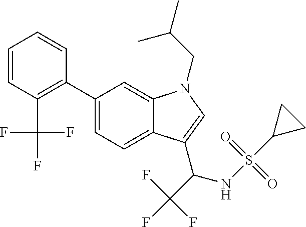 [SMILES: CC(C)Cn1cc(C(NS(=O)(=O)C2CC2)C(F)(F)F)c2ccc(-c3ccccc3C(F)(F)F)cc21] | 208 |
| 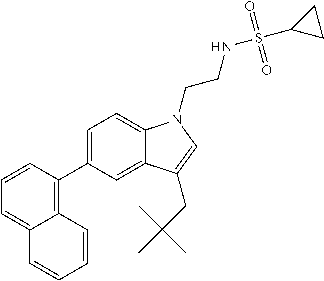 [SMILES: CC(C)(C)Cc1cn(CCNS(=O)(=O)C2CC2)c2ccc(-c3cccc4ccccc34)cc12] | 209 |
| 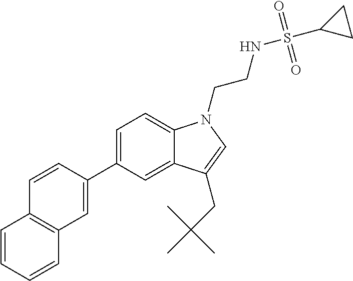 [SMILES: CC(C)(C)Cc1cn(CCNS(=O)(=O)C2CC2)c2ccc(-c3ccc4ccccc4c3)cc12] | 210 |
| 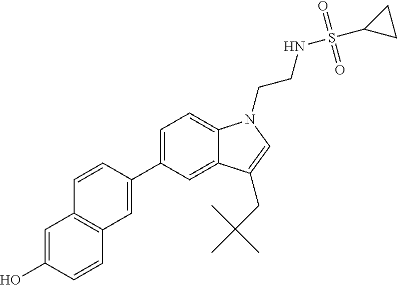 [SMILES: CC(C)(C)Cc1cn(CCNS(=O)(=O)C2CC2)c2ccc(-c3ccc4cc(O)ccc4c3)cc12] | 211 |
| 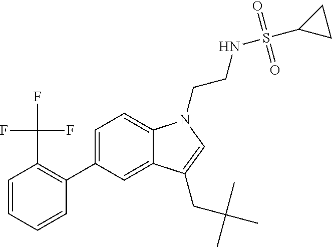 [SMILES: CC(C)(C)Cc1cn(CCNS(=O)(=O)C2CC2)c2ccc(-c3ccccc3C(F)(F)F)cc12] | 212 |
| 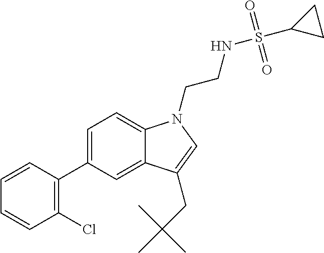 [SMILES: CC(C)(C)Cc1cn(CCNS(=O)(=O)C2CC2)c2ccc(-c3ccccc3Cl)cc12] | 213 |
| 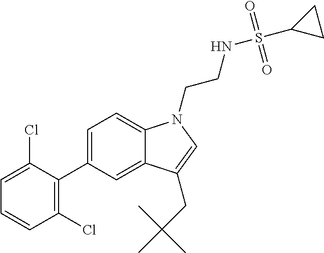 [SMILES: CC(C)(C)Cc1cn(CCNS(=O)(=O)C2CC2)c2ccc(-c3c(Cl)cccc3Cl)cc12] | 214 |
| 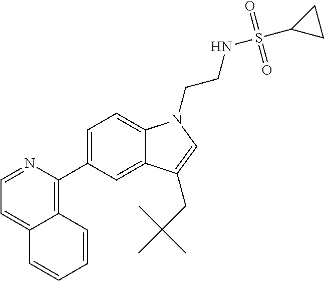 [SMILES: CC(C)(C)Cc1cn(CCNS(=O)(=O)C2CC2)c2ccc(-c3nccc4ccccc34)cc12] | 215 |
| 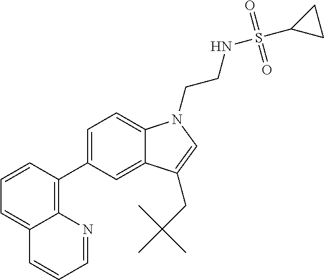 [SMILES: CC(C)(C)Cc1cn(CCNS(=O)(=O)C2CC2)c2ccc(-c3cccc4cccnc34)cc12] | 216 |
| 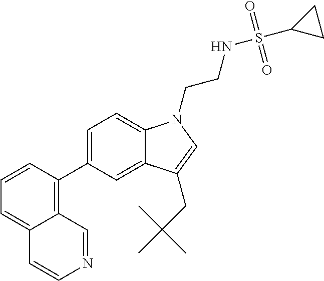 [SMILES: CC(C)(C)Cc1cn(CCNS(=O)(=O)C2CC2)c2ccc(-c3cccc4ccncc34)cc12] | 217 |
| 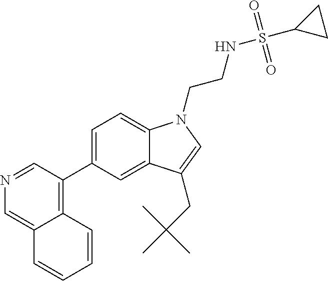 [SMILES: CC(C)(C)Cc1cn(CCNS(=O)(=O)C2CC2)c2ccc(-c3cncc4ccccc34)cc12] | 218 |
| 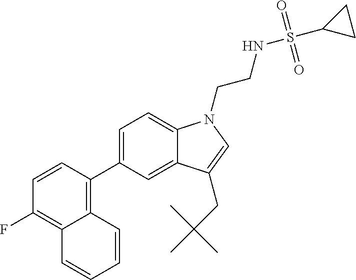 [SMILES: CC(C)(C)Cc1cn(CCNS(=O)(=O)C2CC2)c2ccc(-c3ccc(F)c4ccccc34)cc12] | 219 |
| 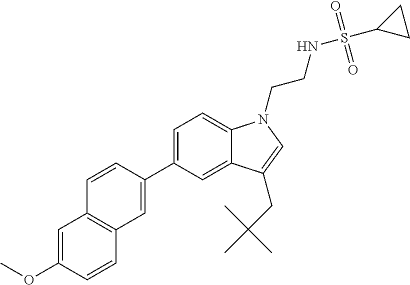 [SMILES: COc1ccc2cc(-c3ccc4c(c3)c(CC(C)(C)C)cn4CCNS(=O)(=O)C3CC3)ccc2c1] | 220 |
| 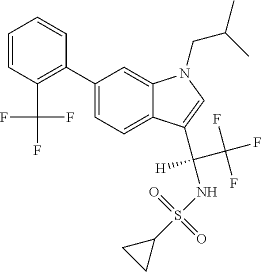 [SMILES: CC(C)Cn1cc([C@H](NS(=O)(=O)C2CC2)C(F)(F)F)c2ccc(-c3ccccc3C(F)(F)F)cc21] | 221 |
| 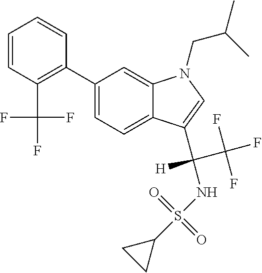 [SMILES: CC(C)Cn1cc([C@@H](NS(=O)(=O)C2CC2)C(F)(F)F)c2ccc(-c3ccccc3C(F)(F)F)cc21] | 222 |
| 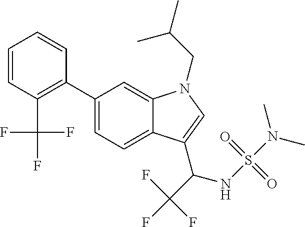 [SMILES: CC(C)Cn1cc(C(NS(=O)(=O)N(C)C)C(F)(F)F)c2ccc(-c3ccccc3C(F)(F)F)cc21] | 223 |
| 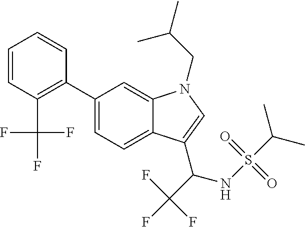 [SMILES: CC(C)Cn1cc(C(NS(=O)(=O)C(C)C)C(F)(F)F)c2ccc(-c3ccccc3C(F)(F)F)cc21] | 224 |
| 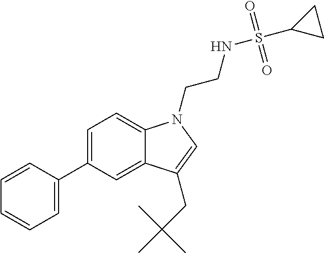 [SMILES: CC(C)(C)Cc1cn(CCNS(=O)(=O)C2CC2)c2ccc(-c3ccccc3)cc12] | 225 |
| 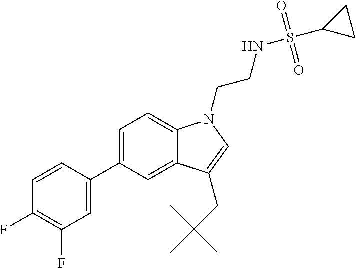 [SMILES: CC(C)(C)Cc1cn(CCNS(=O)(=O)C2CC2)c2ccc(-c3ccc(F)c(F)c3)cc12] | 226 |
| 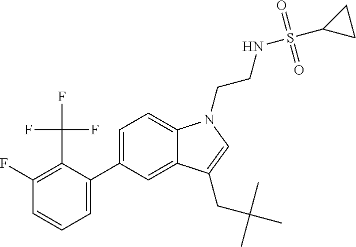 [SMILES: CC(C)(C)Cc1cn(CCNS(=O)(=O)C2CC2)c2ccc(-c3cccc(F)c3C(F)(F)F)cc12] | 227 |
| 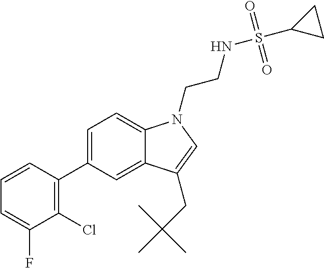 [SMILES: CC(C)(C)Cc1cn(CCNS(=O)(=O)C2CC2)c2ccc(-c3cccc(F)c3Cl)cc12] | 228 |
| 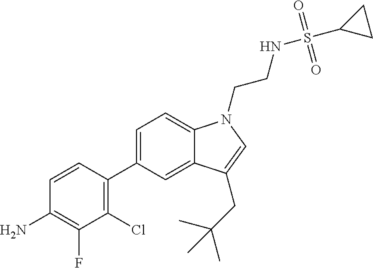 [SMILES: CC(C)(C)Cc1cn(CCNS(=O)(=O)C2CC2)c2ccc(-c3ccc(N)c(F)c3Cl)cc12] | 229 |
| 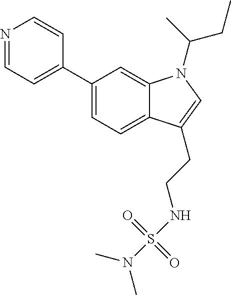 [SMILES: CCC(C)n1cc(CCNS(=O)(=O)N(C)C)c2ccc(-c3ccncc3)cc21] | 230 |
| 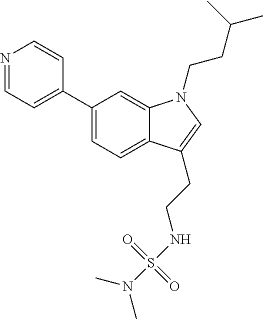 [SMILES: CC(C)CCn1cc(CCNS(=O)(=O)N(C)C)c2ccc(-c3ccncc3)cc21] | 231 |
| 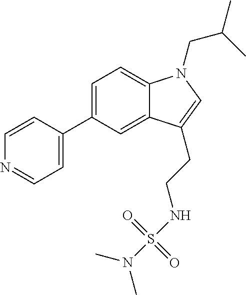 [SMILES: CC(C)Cn1cc(CCNS(=O)(=O)N(C)C)c2cc(-c3ccncc3)ccc21] | 232 |
| 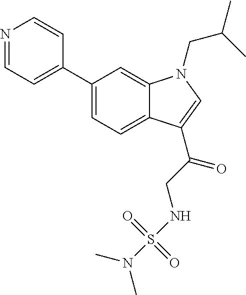 [SMILES: CC(C)Cn1cc(C(=O)CNS(=O)(=O)N(C)C)c2ccc(-c3ccncc3)cc21] | 233 |
| 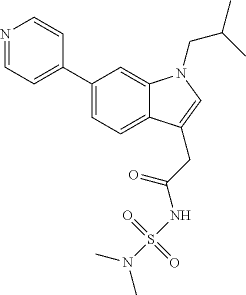 [SMILES: CC(C)Cn1cc(CC(=O)NS(=O)(=O)N(C)C)c2ccc(-c3ccncc3)cc21] | 234 |
| 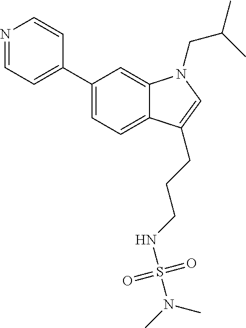 [SMILES: CC(C)Cn1cc(CCCNS(=O)(=O)N(C)C)c2ccc(-c3ccncc3)cc21] | 235 |
| 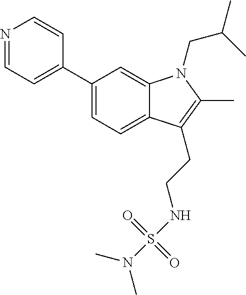 [SMILES: Cc1c(CCNS(=O)(=O)N(C)C)c2ccc(-c3ccncc3)cc2n1CC(C)C] | 236 |
| 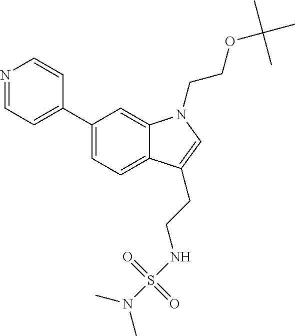 [SMILES: CN(C)S(=O)(=O)NCCc1cn(CCOC(C)(C)C)c2cc(-c3ccncc3)ccc12] | 237 |
| 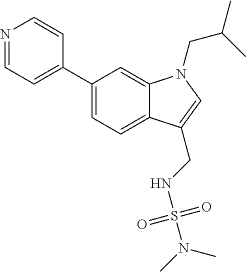 [SMILES: CC(C)Cn1cc(CNS(=O)(=O)N(C)C)c2ccc(-c3ccncc3)cc21] | 238 |
| 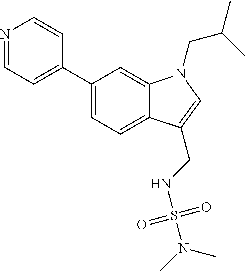 [SMILES: CC(C)Cn1cc(CNS(=O)(=O)N(C)C)c2ccc(-c3ccncc3)cc21] | 239 |
| 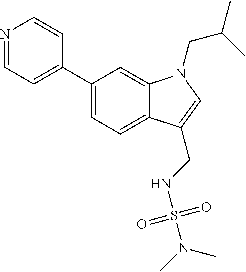 [SMILES: CC(C)Cn1cc(CNS(=O)(=O)N(C)C)c2ccc(-c3ccncc3)cc21] | 242 |
| 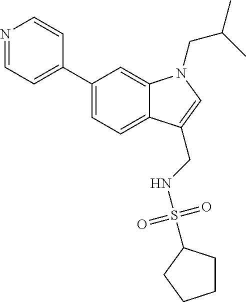 [SMILES: CC(C)Cn1cc(CNS(=O)(=O)C2CCCC2)c2ccc(-c3ccncc3)cc21] | 243 |
| 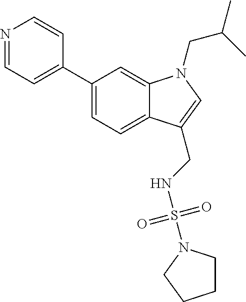 [SMILES: CC(C)Cn1cc(CNS(=O)(=O)N2CCCC2)c2ccc(-c3ccncc3)cc21] | 244 |
| 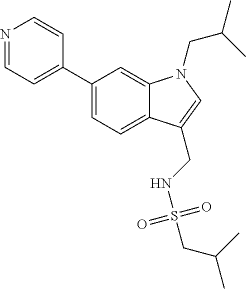 [SMILES: CC(C)Cn1cc(CNS(=O)(=O)CC(C)C)c2ccc(-c3ccncc3)cc21] | 245 |
| 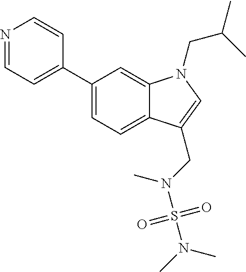 [SMILES: CC(C)Cn1cc(CN(C)S(=O)(=O)N(C)C)c2ccc(-c3ccncc3)cc21] | 246 |
| 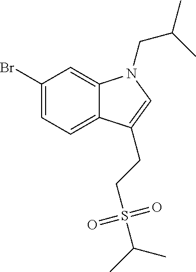 [SMILES: CC(C)Cn1cc(CCS(=O)(=O)C(C)C)c2ccc(Br)cc21] | 247 |
| 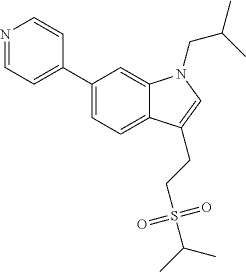 [SMILES: CC(C)Cn1cc(CCS(=O)(=O)C(C)C)c2ccc(-c3ccncc3)cc21] | 248 |
| 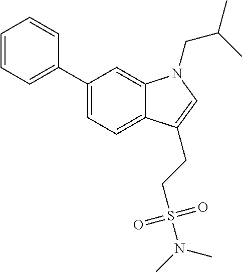 [SMILES: CC(C)Cn1cc(CCS(=O)(=O)N(C)C)c2ccc(-c3ccccc3)cc21] | 249 |
| 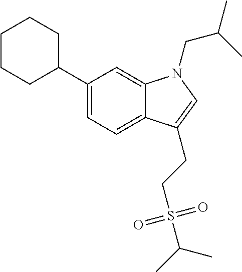 [SMILES: CC(C)Cn1cc(CCS(=O)(=O)C(C)C)c2ccc(C3CCCCC3)cc21] | 250 |
| 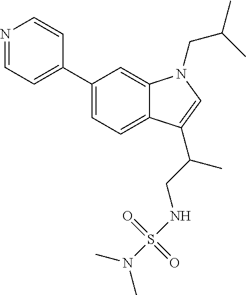 [SMILES: CC(C)Cn1cc(C(C)CNS(=O)(=O)N(C)C)c2ccc(-c3ccncc3)cc21] | 251 |
| 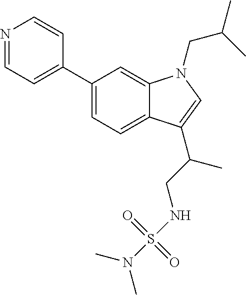 [SMILES: CC(C)Cn1cc(C(C)CNS(=O)(=O)N(C)C)c2ccc(-c3ccncc3)cc21] | 252 |
| 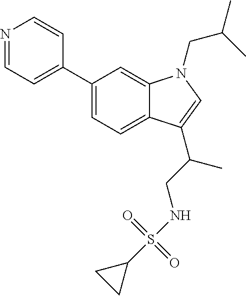 [SMILES: CC(C)Cn1cc(C(C)CNS(=O)(=O)C2CC2)c2ccc(-c3ccncc3)cc21] | 253 |
| 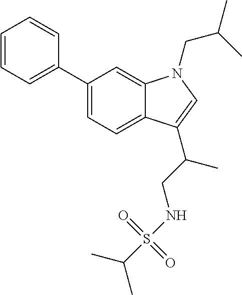 [SMILES: CC(C)Cn1cc(C(C)CNS(=O)(=O)C(C)C)c2ccc(-c3ccccc3)cc21] | 254 |
| 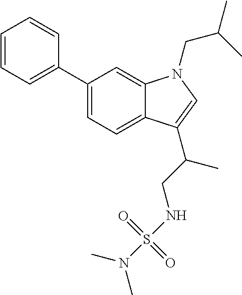 [SMILES: CC(C)Cn1cc(C(C)CNS(=O)(=O)N(C)C)c2ccc(-c3ccccc3)cc21] | 255 |
| 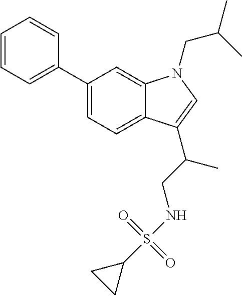 [SMILES: CC(C)Cn1cc(C(C)CNS(=O)(=O)C2CC2)c2ccc(-c3ccccc3)cc21] | 256 |
| 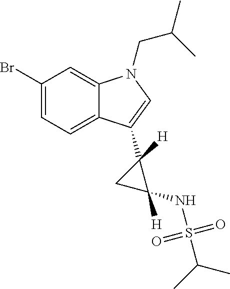 | 257 |
| 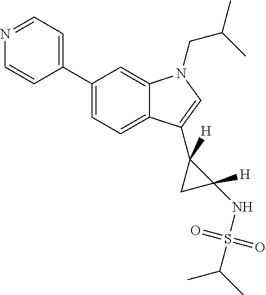 [SMILES: CC(C)Cn1cc([C@H]2C[C@H]2NS(=O)(=O)C(C)C)c2ccc(-c3ccncc3)cc21] | 258 |
| 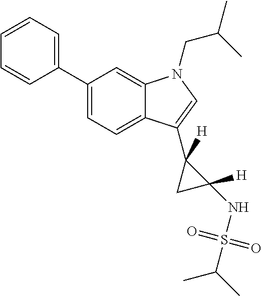 [SMILES: CC(C)Cn1cc([C@H]2C[C@H]2NS(=O)(=O)C(C)C)c2ccc(-c3ccccc3)cc21] | 259 |
| 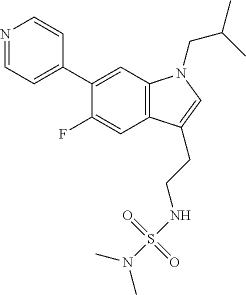 [SMILES: CC(C)Cn1cc(CCNS(=O)(=O)N(C)C)c2cc(F)c(-c3ccncc3)cc21] | 260 |
| 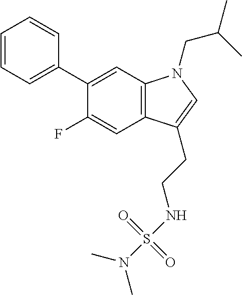 [SMILES: CC(C)Cn1cc(CCNS(=O)(=O)N(C)C)c2cc(F)c(-c3ccccc3)cc21] | 261 |
| 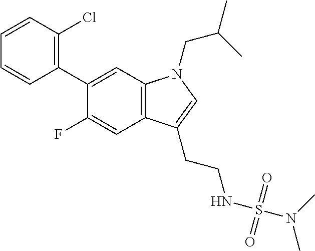 [SMILES: CC(C)Cn1cc(CCNS(=O)(=O)N(C)C)c2cc(F)c(-c3ccccc3Cl)cc21] | 262 |
| 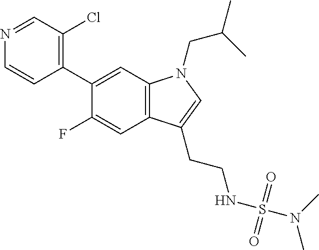 [SMILES: CC(C)Cn1cc(CCNS(=O)(=O)N(C)C)c2cc(F)c(-c3ccncc3Cl)cc21] | 263 |
| 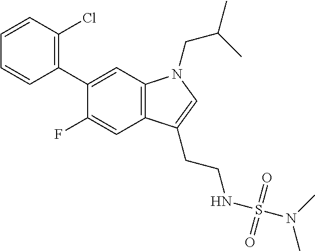 [SMILES: CC(C)Cn1cc(CCNS(=O)(=O)N(C)C)c2cc(F)c(-c3ccccc3Cl)cc21] | 264 |
| 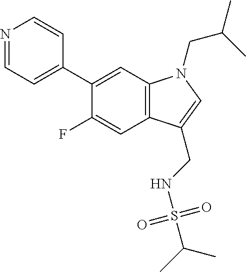 [SMILES: CC(C)Cn1cc(CNS(=O)(=O)C(C)C)c2cc(F)c(-c3ccncc3)cc21] | 265 |
| 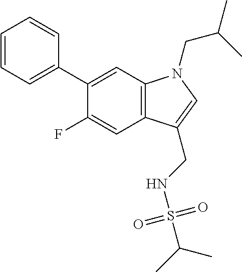 [SMILES: CC(C)Cn1cc(CNS(=O)(=O)C(C)C)c2cc(F)c(-c3ccccc3)cc21] | 266 |
| 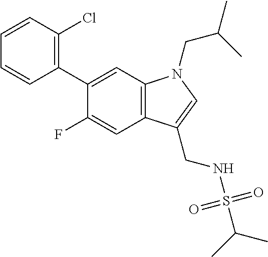 [SMILES: CC(C)Cn1cc(CNS(=O)(=O)C(C)C)c2cc(F)c(-c3ccccc3Cl)cc21] | 267 |
| 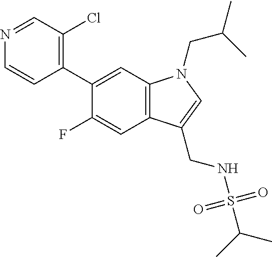 [SMILES: CC(C)Cn1cc(CNS(=O)(=O)C(C)C)c2cc(F)c(-c3ccncc3Cl)cc21] | 268 |
| 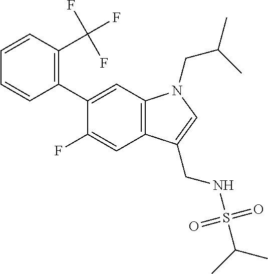 [SMILES: CC(C)Cn1cc(CNS(=O)(=O)C(C)C)c2cc(F)c(-c3ccccc3C(F)(F)F)cc21] | 269 |
| 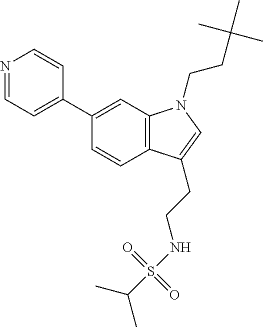 [SMILES: CC(C)S(=O)(=O)NCCc1cn(CCC(C)(C)C)c2cc(-c3ccncc3)ccc12] | 270 |
| 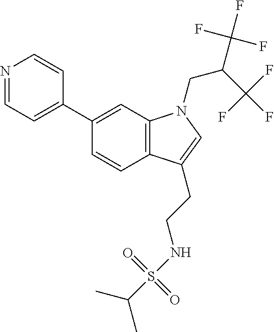 [SMILES: CC(C)S(=O)(=O)NCCc1cn(CC(C(F)(F)F)C(F)(F)F)c2cc(-c3ccncc3)ccc12] | 271 |
| 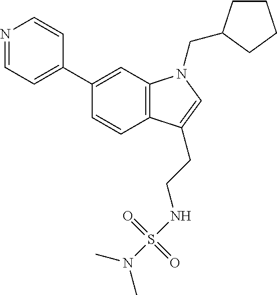 [SMILES: CN(C)S(=O)(=O)NCCc1cn(CC2CCCC2)c2cc(-c3ccncc3)ccc12] | 272 |
| 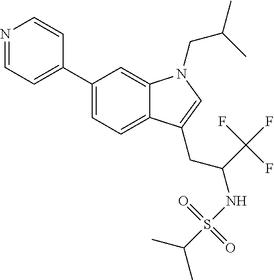 [SMILES: CC(C)Cn1cc(CC(NS(=O)(=O)C(C)C)C(F)(F)F)c2ccc(-c3ccncc3)cc21] | 273 |
| 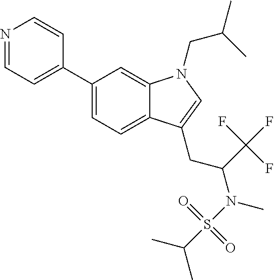 [SMILES: CC(C)Cn1cc(CC(N(C)S(=O)(=O)C(C)C)C(F)(F)F)c2ccc(-c3ccncc3)cc21] | 274 |
| 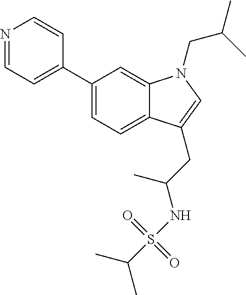 [SMILES: CC(C)Cn1cc(CC(C)NS(=O)(=O)C(C)C)c2ccc(-c3ccncc3)cc21] | 275 |
| 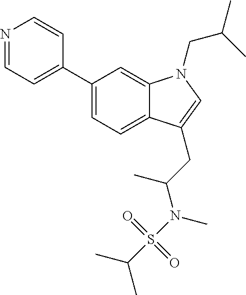 [SMILES: CC(C)Cn1cc(CC(C)N(C)S(=O)(=O)C(C)C)c2ccc(-c3ccncc3)cc21] | 276 |
| 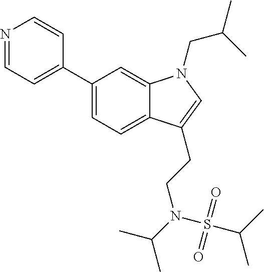 [SMILES: CC(C)Cn1cc(CCN(C(C)C)S(=O)(=O)C(C)C)c2ccc(-c3ccncc3)cc21] | 277 |
| 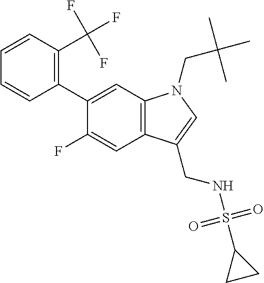 [SMILES: CC(C)(C)Cn1cc(CNS(=O)(=O)C2CC2)c2cc(F)c(-c3ccccc3C(F)(F)F)cc21] | 278 |
| 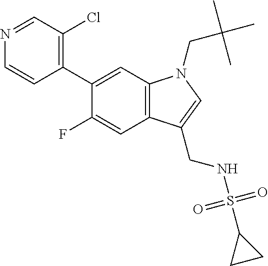 [SMILES: CC(C)(C)Cn1cc(CNS(=O)(=O)C2CC2)c2cc(F)c(-c3ccncc3Cl)cc21] | 279 |
| 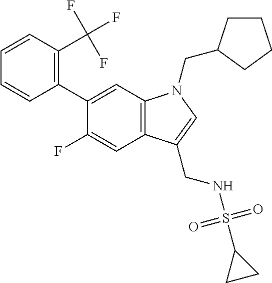 [SMILES: O=S(=O)(NCc1cn(CC2CCCC2)c2cc(-c3ccccc3C(F)(F)F)c(F)cc12)C1CC1] | 280 |
| 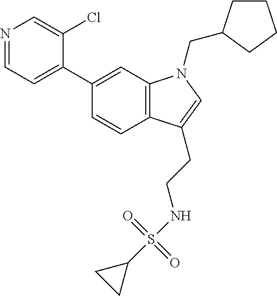 [SMILES: O=S(=O)(NCCc1cn(CC2CCCC2)c2cc(-c3ccncc3Cl)ccc12)C1CC1] | 281 |
| 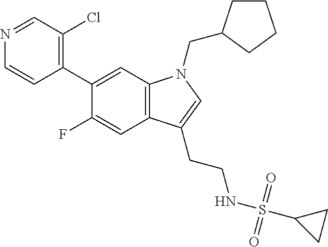 [SMILES: O=S(=O)(NCCc1cn(CC2CCCC2)c2cc(-c3ccncc3Cl)c(F)cc12)C1CC1] | 282 |
| 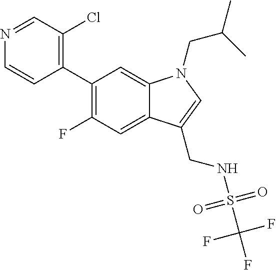 [SMILES: CC(C)Cn1cc(CNS(=O)(=O)C(F)(F)F)c2cc(F)c(-c3ccncc3Cl)cc21] | 283 |
| 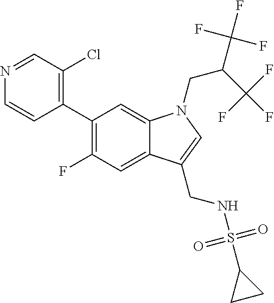 [SMILES: O=S(=O)(NCc1cn(CC(C(F)(F)F)C(F)(F)F)c2cc(-c3ccncc3Cl)c(F)cc12)C1CC1] | 284 |
| 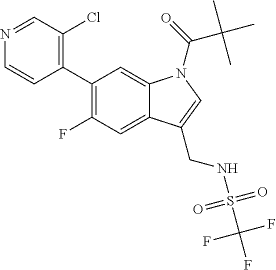 [SMILES: CC(C)(C)C(=O)n1cc(CNS(=O)(=O)C(F)(F)F)c2cc(F)c(-c3ccncc3Cl)cc21] | 285 |
| 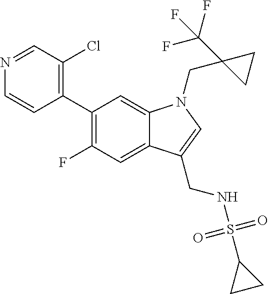 [SMILES: O=S(=O)(NCc1cn(CC2(C(F)(F)F)CC2)c2cc(-c3ccncc3Cl)c(F)cc12)C1CC1] | 286 |
| 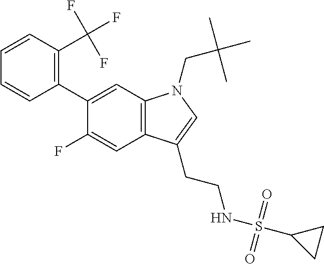 [SMILES: CC(C)(C)Cn1cc(CCNS(=O)(=O)C2CC2)c2cc(F)c(-c3ccccc3C(F)(F)F)cc21] | 287 |
| 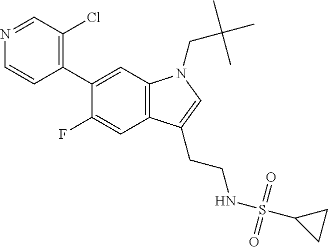 [SMILES: CC(C)(C)Cn1cc(CCNS(=O)(=O)C2CC2)c2cc(F)c(-c3ccncc3Cl)cc21] | 288 |
| 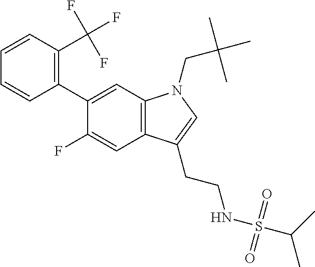 [SMILES: CC(C)S(=O)(=O)NCCc1cn(CC(C)(C)C)c2cc(-c3ccccc3C(F)(F)F)c(F)cc12] | 289 |
| 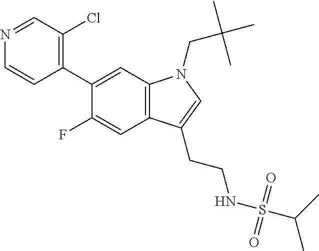 [SMILES: CC(C)S(=O)(=O)NCCc1cn(CC(C)(C)C)c2cc(-c3ccncc3Cl)c(F)cc12] | 290 |
| 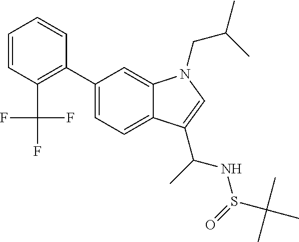 [SMILES: CC(C)Cn1cc(C(C)NS(=O)C(C)(C)C)c2ccc(-c3ccccc3C(F)(F)F)cc21] | 291 |
| 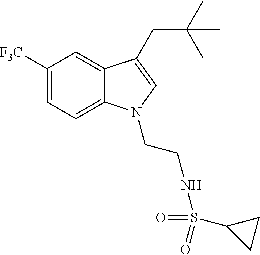 [SMILES: CC(C)(C)Cc1cn(CCNS(=O)(=O)C2CC2)c2ccc(C(F)(F)F)cc12] | 292 |
| 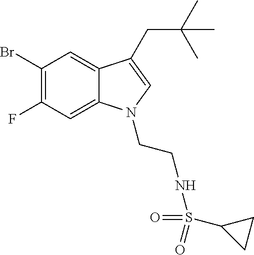 [SMILES: CC(C)(C)Cc1cn(CCNS(=O)(=O)C2CC2)c2cc(F)c(Br)cc12] | 293 |
| 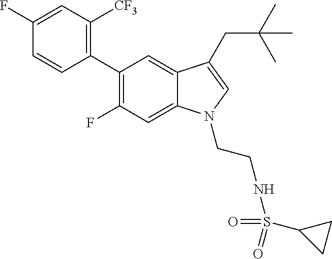 [SMILES: CC(C)(C)Cc1cn(CCNS(=O)(=O)C2CC2)c2cc(F)c(-c3ccc(F)cc3C(F)(F)F)cc12] | 294 |
| 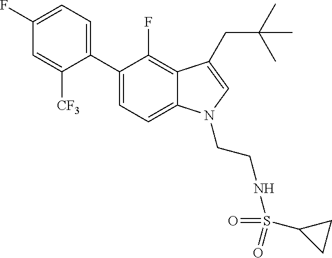 [SMILES: CC(C)(C)Cc1cn(CCNS(=O)(=O)C2CC2)c2ccc(-c3ccc(F)cc3C(F)(F)F)c(F)c12] | 295 |
| 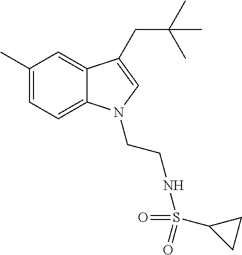 [SMILES: Cc1ccc2c(c1)c(CC(C)(C)C)cn2CCNS(=O)(=O)C1CC1] | 296 |
| 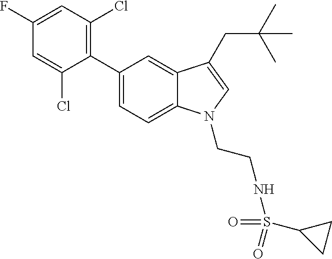 [SMILES: CC(C)(C)Cc1cn(CCNS(=O)(=O)C2CC2)c2ccc(-c3c(Cl)cc(F)cc3Cl)cc12] | 297 |
| 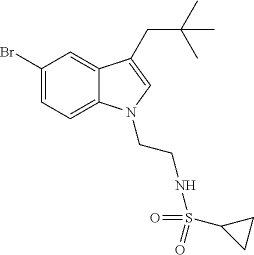 [SMILES: CC(C)(C)Cc1cn(CCNS(=O)(=O)C2CC2)c2ccc(Br)cc12] | 298 |
| 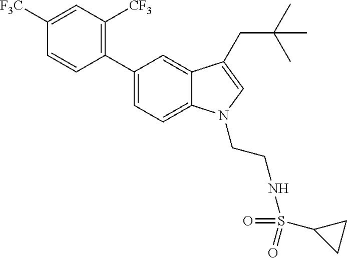 [SMILES: CC(C)(C)Cc1cn(CCNS(=O)(=O)C2CC2)c2ccc(-c3ccc(C(F)(F)F)cc3C(F)(F)F)cc12] | 299 |
| 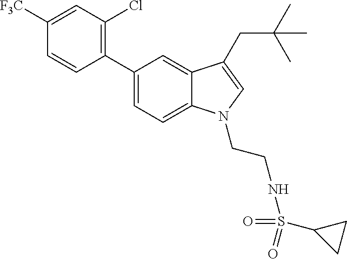 [SMILES: CC(C)(C)Cc1cn(CCNS(=O)(=O)C2CC2)c2ccc(-c3ccc(C(F)(F)F)cc3Cl)cc12] | 300 |
| 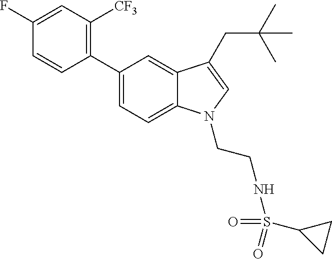 [SMILES: CC(C)(C)Cc1cn(CCNS(=O)(=O)C2CC2)c2ccc(-c3ccc(F)cc3C(F)(F)F)cc12] | 301 |
| 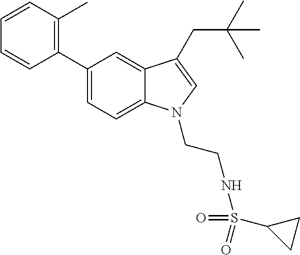 [SMILES: Cc1ccccc1-c1ccc2c(c1)c(CC(C)(C)C)cn2CCNS(=O)(=O)C1CC1] | 302 |
| 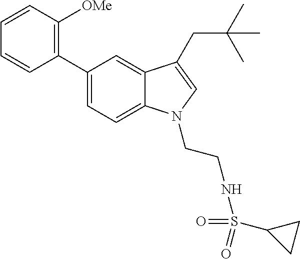 [SMILES: COc1ccccc1-c1ccc2c(c1)c(CC(C)(C)C)cn2CCNS(=O)(=O)C1CC1] | 303 |
| 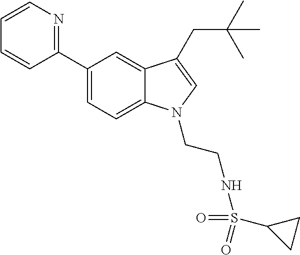 [SMILES: CC(C)(C)Cc1cn(CCNS(=O)(=O)C2CC2)c2ccc(-c3ccccn3)cc12] | 304 |
| 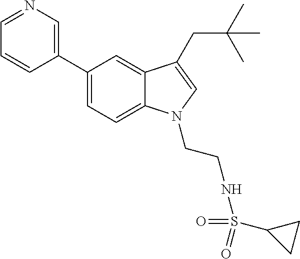 [SMILES: CC(C)(C)Cc1cn(CCNS(=O)(=O)C2CC2)c2ccc(-c3cccnc3)cc12] | 305 |
| 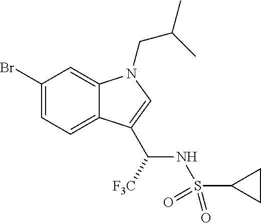 [SMILES: CC(C)Cn1cc([C@H](NS(=O)(=O)C2CC2)C(F)(F)F)c2ccc(Br)cc21] | 306 |
| 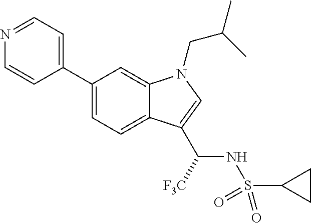 [SMILES: CC(C)Cn1cc([C@H](NS(=O)(=O)C2CC2)C(F)(F)F)c2ccc(-c3ccncc3)cc21] | 307 |
| 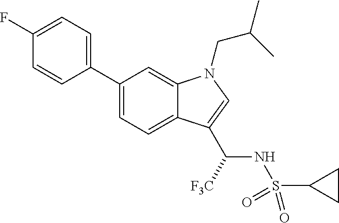 [SMILES: CC(C)Cn1cc([C@H](NS(=O)(=O)C2CC2)C(F)(F)F)c2ccc(-c3ccc(F)cc3)cc21] | 308 |
| 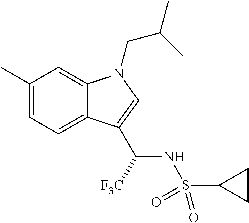 [SMILES: Cc1ccc2c([C@H](NS(=O)(=O)C3CC3)C(F)(F)F)cn(CC(C)C)c2c1] | 309 |
| 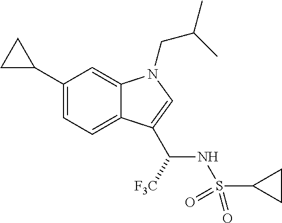 [SMILES: CC(C)Cn1cc([C@H](NS(=O)(=O)C2CC2)C(F)(F)F)c2ccc(C3CC3)cc21] | 310 |
| 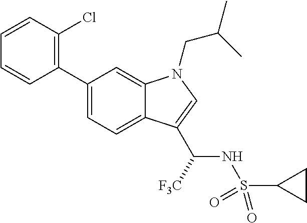 [SMILES: CC(C)Cn1cc([C@H](NS(=O)(=O)C2CC2)C(F)(F)F)c2ccc(-c3ccccc3Cl)cc21] | 311 |
| 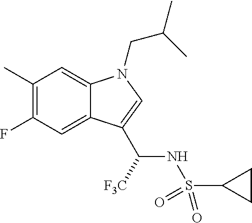 [SMILES: Cc1cc2c(cc1F)c([C@H](NS(=O)(=O)C1CC1)C(F)(F)F)cn2CC(C)C] | 312 |
| 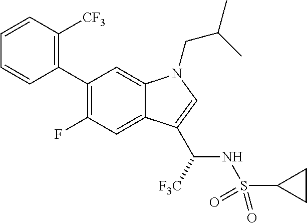 [SMILES: CC(C)Cn1cc([C@H](NS(=O)(=O)C2CC2)C(F)(F)F)c2cc(F)c(-c3ccccc3C(F)(F)F)cc21] | 313 |
| 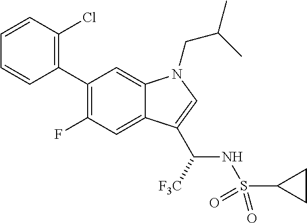 [SMILES: CC(C)Cn1cc([C@H](NS(=O)(=O)C2CC2)C(F)(F)F)c2cc(F)c(-c3ccccc3Cl)cc21] | 314 |
| 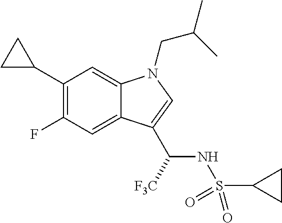 [SMILES: CC(C)Cn1cc([C@H](NS(=O)(=O)C2CC2)C(F)(F)F)c2cc(F)c(C3CC3)cc21] | 315 |
| 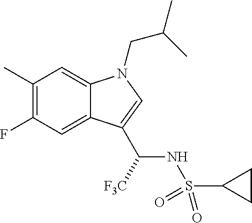 [SMILES: Cc1cc2c(cc1F)c([C@H](NS(=O)(=O)C1CC1)C(F)(F)F)cn2CC(C)C] | 316 |
| 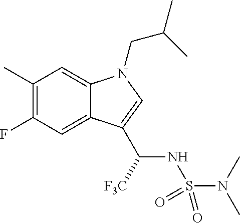 | 317 |
| 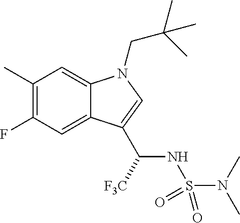 | 318 |
| 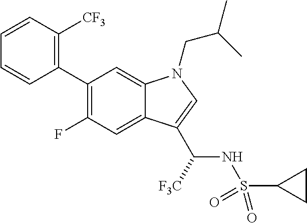 [SMILES: CC(C)Cn1cc([C@H](NS(=O)(=O)C2CC2)C(F)(F)F)c2cc(F)c(-c3ccccc3C(F)(F)F)cc21] | 319 |
| 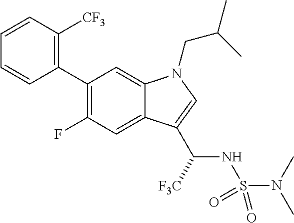 [SMILES: CC(C)Cn1cc([C@H](NS(=O)(=O)N(C)C)C(F)(F)F)c2cc(F)c(-c3ccccc3C(F)(F)F)cc21] | 320 |
| 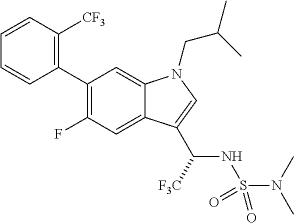 [SMILES: CC(C)Cn1cc([C@H](NS(=O)(=O)N(C)C)C(F)(F)F)c2cc(F)c(-c3ccccc3C(F)(F)F)cc21] | 321 |
| 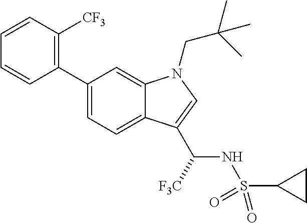 [SMILES: CC(C)(C)Cn1cc([C@H](NS(=O)(=O)C2CC2)C(F)(F)F)c2ccc(-c3ccccc3C(F)(F)F)cc21] | 322 |
| 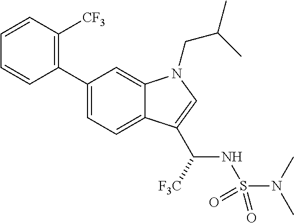 [SMILES: CC(C)Cn1cc([C@H](NS(=O)(=O)N(C)C)C(F)(F)F)c2ccc(-c3ccccc3C(F)(F)F)cc21] | 323 |
| 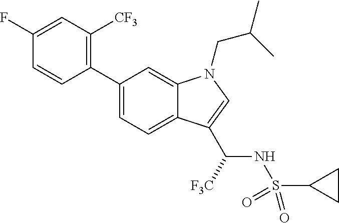 [SMILES: CC(C)Cn1cc([C@H](NS(=O)(=O)C2CC2)C(F)(F)F)c2ccc(-c3ccc(F)cc3C(F)(F)F)cc21] | 324 |
| 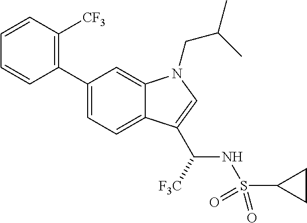 [SMILES: CC(C)Cn1cc([C@H](NS(=O)(=O)C2CC2)C(F)(F)F)c2ccc(-c3ccccc3C(F)(F)F)cc21] | 325 |
| 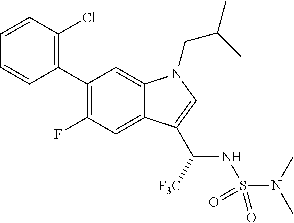 [SMILES: CC(C)Cn1cc([C@H](NS(=O)(=O)N(C)C)C(F)(F)F)c2cc(F)c(-c3ccccc3Cl)cc21] | 326 |
| 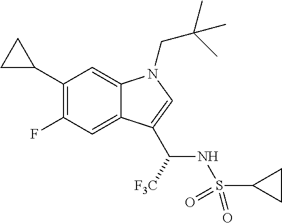 [SMILES: CC(C)(C)Cn1cc([C@H](NS(=O)(=O)C2CC2)C(F)(F)F)c2cc(F)c(C3CC3)cc21] | 327 |
| 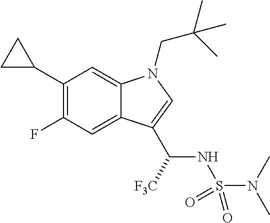 [SMILES: CN(C)S(=O)(=O)N[C@@H](c1cn(CC(C)(C)C)c2cc(C3CC3)c(F)cc12)C(F)(F)F] | 328 |
| 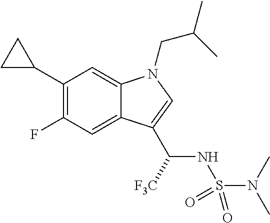 [SMILES: CC(C)Cn1cc([C@H](NS(=O)(=O)N(C)C)C(F)(F)F)c2cc(F)c(C3CC3)cc21] | 329 |
| 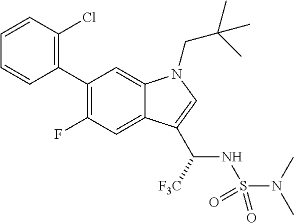 [SMILES: CN(C)S(=O)(=O)N[C@@H](c1cn(CC(C)(C)C)c2cc(-c3ccccc3Cl)c(F)cc12)C(F)(F)F] | 330 |
| 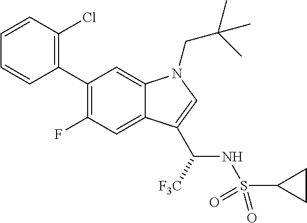 [SMILES: CC(C)(C)Cn1cc([C@H](NS(=O)(=O)C2CC2)C(F)(F)F)c2cc(F)c(-c3ccccc3Cl)cc21] | 331 |
| 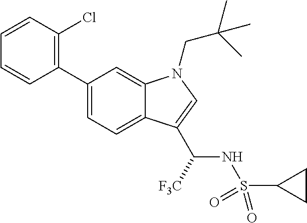 [SMILES: CC(C)(C)Cn1cc([C@H](NS(=O)(=O)C2CC2)C(F)(F)F)c2ccc(-c3ccccc3Cl)cc21] | 332 |
| 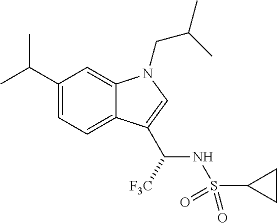 [SMILES: CC(C)Cn1cc([C@H](NS(=O)(=O)C2CC2)C(F)(F)F)c2ccc(C(C)C)cc21] | 333 |
| 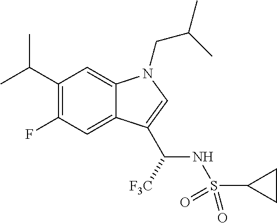 [SMILES: CC(C)Cn1cc([C@H](NS(=O)(=O)C2CC2)C(F)(F)F)c2cc(F)c(C(C)C)cc21] | 334 |
| 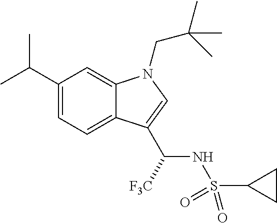 [SMILES: CC(C)c1ccc2c([C@H](NS(=O)(=O)C3CC3)C(F)(F)F)cn(CC(C)(C)C)c2c1] | 335 |
| 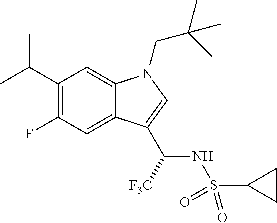 [SMILES: CC(C)c1cc2c(cc1F)c([C@H](NS(=O)(=O)C1CC1)C(F)(F)F)cn2CC(C)(C)C] | 336 |
| 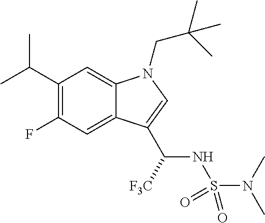 [SMILES: CC(C)c1cc2c(cc1F)c([C@H](NS(=O)(=O)N(C)C)C(F)(F)F)cn2CC(C)(C)C] | 337 |
| 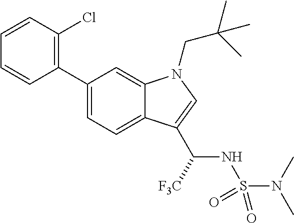 [SMILES: CN(C)S(=O)(=O)N[C@@H](c1cn(CC(C)(C)C)c2cc(-c3ccccc3Cl)ccc12)C(F)(F)F] | 338 |
| 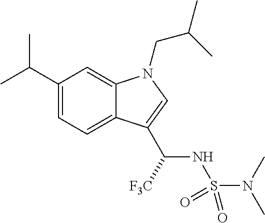 [SMILES: CC(C)Cn1cc([C@H](NS(=O)(=O)N(C)C)C(F)(F)F)c2ccc(C(C)C)cc21] | 339 |
| 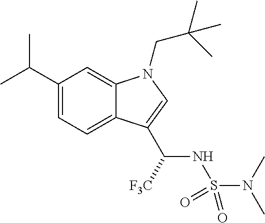 [SMILES: CC(C)c1ccc2c([C@H](NS(=O)(=O)N(C)C)C(F)(F)F)cn(CC(C)(C)C)c2c1] | 340 |
| 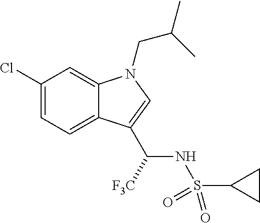 [SMILES: CC(C)Cn1cc([C@H](NS(=O)(=O)C2CC2)C(F)(F)F)c2ccc(Cl)cc21] | 341 |
| 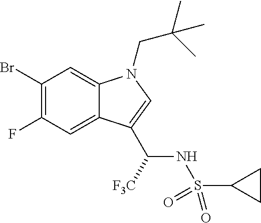 [SMILES: CC(C)(C)Cn1cc([C@H](NS(=O)(=O)C2CC2)C(F)(F)F)c2cc(F)c(Br)cc21] | 342 |
| 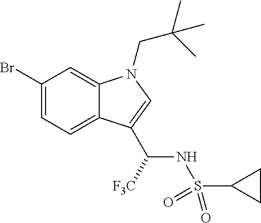 [SMILES: CC(C)(C)Cn1cc([C@H](NS(=O)(=O)C2CC2)C(F)(F)F)c2ccc(Br)cc21] | 343 |
| 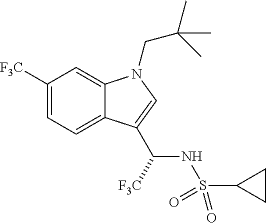 [SMILES: CC(C)(C)Cn1cc([C@H](NS(=O)(=O)C2CC2)C(F)(F)F)c2ccc(C(F)(F)F)cc21] | 344 |
| 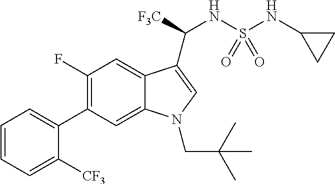 [SMILES: CC(C)(C)Cn1cc([C@H](NS(=O)(=O)NC2CC2)C(F)(F)F)c2cc(F)c(-c3ccccc3C(F)(F)F)cc21] | 345 |
| 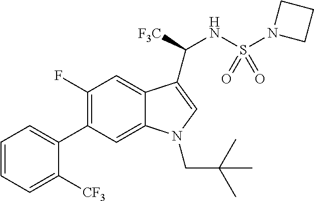 [SMILES: CC(C)(C)Cn1cc([C@H](NS(=O)(=O)N2CCC2)C(F)(F)F)c2cc(F)c(-c3ccccc3C(F)(F)F)cc21] | 346 |
| 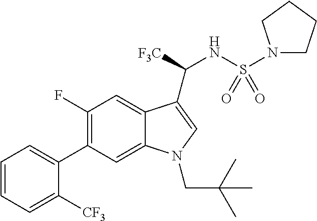 [SMILES: CC(C)(C)Cn1cc([C@H](NS(=O)(=O)N2CCCC2)C(F)(F)F)c2cc(F)c(-c3ccccc3C(F)(F)F)cc21] | 347 |
| 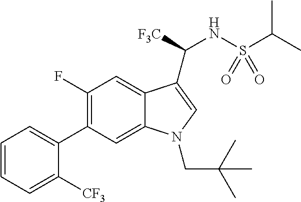 [SMILES: CC(C)S(=O)(=O)N[C@@H](c1cn(CC(C)(C)C)c2cc(-c3ccccc3C(F)(F)F)c(F)cc12)C(F)(F)F] | 348 |
| 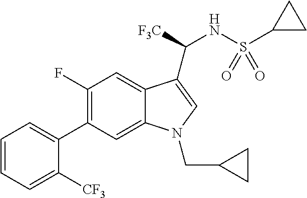 [SMILES: O=S(=O)(N[C@@H](c1cn(CC2CC2)c2cc(-c3ccccc3C(F)(F)F)c(F)cc12)C(F)(F)F)C1CC1] | 349 |
| 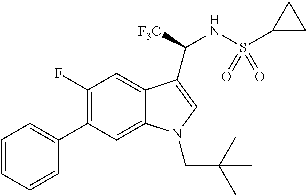 [SMILES: CC(C)(C)Cn1cc([C@H](NS(=O)(=O)C2CC2)C(F)(F)F)c2cc(F)c(-c3ccccc3)cc21] | 350 |
| 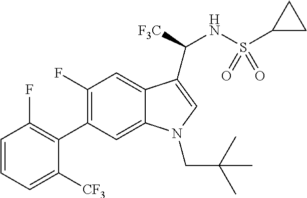 [SMILES: CC(C)(C)Cn1cc([C@H](NS(=O)(=O)C2CC2)C(F)(F)F)c2cc(F)c(-c3c(F)cccc3C(F)(F)F)cc21] | 351 |
| 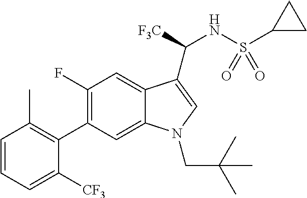 [SMILES: Cc1cccc(C(F)(F)F)c1-c1cc2c(cc1F)c([C@H](NS(=O)(=O)C1CC1)C(F)(F)F)cn2CC(C)(C)C] | 352 |
| 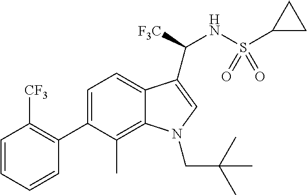 [SMILES: Cc1c(-c2ccccc2C(F)(F)F)ccc2c([C@H](NS(=O)(=O)C3CC3)C(F)(F)F)cn(CC(C)(C)C)c12] | 353 |
| 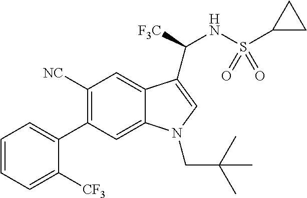 [SMILES: CC(C)(C)Cn1cc([C@H](NS(=O)(=O)C2CC2)C(F)(F)F)c2cc(C#N)c(-c3ccccc3C(F)(F)F)cc21] | 354 |
| 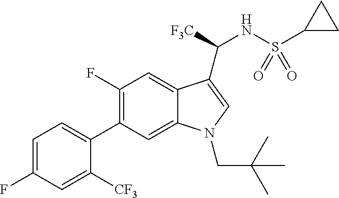 [SMILES: CC(C)(C)Cn1cc([C@H](NS(=O)(=O)C2CC2)C(F)(F)F)c2cc(F)c(-c3ccc(F)cc3C(F)(F)F)cc21] | 355 |
| 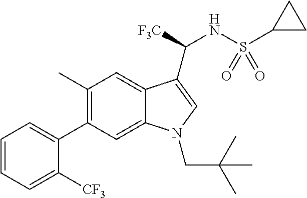 [SMILES: Cc1cc2c([C@H](NS(=O)(=O)C3CC3)C(F)(F)F)cn(CC(C)(C)C)c2cc1-c1ccccc1C(F)(F)F] | 356 |
| 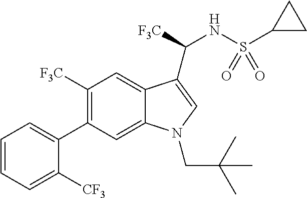 [SMILES: CC(C)(C)Cn1cc([C@H](NS(=O)(=O)C2CC2)C(F)(F)F)c2cc(C(F)(F)F)c(-c3ccccc3C(F)(F)F)cc21] | 357 |
| 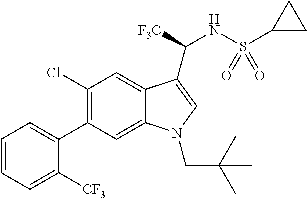 [SMILES: CC(C)(C)Cn1cc([C@H](NS(=O)(=O)C2CC2)C(F)(F)F)c2cc(Cl)c(-c3ccccc3C(F)(F)F)cc21] | 358 |
| 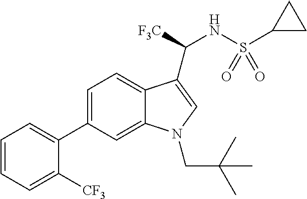 [SMILES: CC(C)(C)Cn1cc([C@H](NS(=O)(=O)C2CC2)C(F)(F)F)c2ccc(-c3ccccc3C(F)(F)F)cc21] | 359 |
| 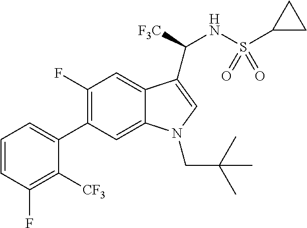 [SMILES: CC(C)(C)Cn1cc([C@H](NS(=O)(=O)C2CC2)C(F)(F)F)c2cc(F)c(-c3cccc(F)c3C(F)(F)F)cc21] | 360 |
| 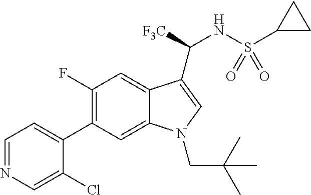 [SMILES: CC(C)(C)Cn1cc([C@H](NS(=O)(=O)C2CC2)C(F)(F)F)c2cc(F)c(-c3ccncc3Cl)cc21] | 361 |
| 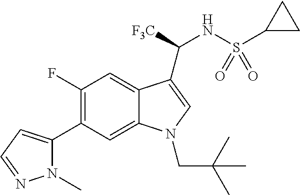 [SMILES: Cn1nccc1-c1cc2c(cc1F)c([C@H](NS(=O)(=O)C1CC1)C(F)(F)F)cn2CC(C)(C)C] | 362 |
| 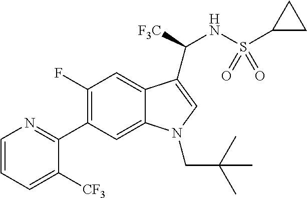 [SMILES: CC(C)(C)Cn1cc([C@H](NS(=O)(=O)C2CC2)C(F)(F)F)c2cc(F)c(-c3ncccc3C(F)(F)F)cc21] | 363 |
| 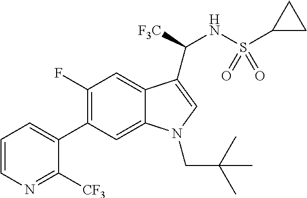 [SMILES: CC(C)(C)Cn1cc([C@H](NS(=O)(=O)C2CC2)C(F)(F)F)c2cc(F)c(-c3cccnc3C(F)(F)F)cc21] | 364 |
| 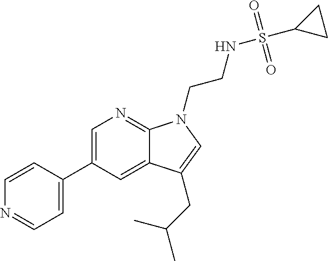 [SMILES: CC(C)Cc1cn(CCNS(=O)(=O)C2CC2)c2ncc(-c3ccncc3)cc12] | 365 |

32. A compound or pharmaceutically acceptable salt thereof according to claim 1 that is selected from the following table:

| | |
|---|---|
| 366 | 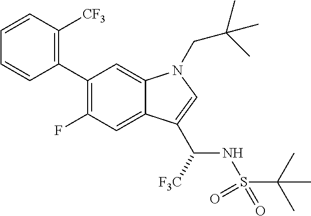 [SMILES: CC(C)(C)Cn1cc([C@H](NS(=O)(=O)C(C)(C)C)C(F)(F)F)c2cc(F)c(-c3ccccc3C(F)(F)F)cc21] |
| 367 | 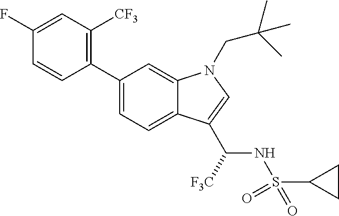 [SMILES: CC(C)(C)Cn1cc([C@H](NS(=O)(=O)C2CC2)C(F)(F)F)c2ccc(-c3ccc(F)cc3C(F)(F)F)cc21] |
| 368 | 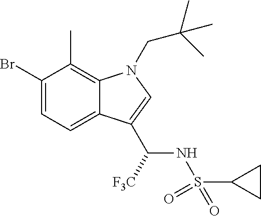 [SMILES: Cc1c(Br)ccc2c([C@H](NS(=O)(=O)C3CC3)C(F)(F)F)cn(CC(C)(C)C)c12] |
| 369 | 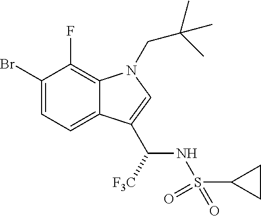 [SMILES: CC(C)(C)Cn1cc([C@H](NS(=O)(=O)C2CC2)C(F)(F)F)c2ccc(Br)c(F)c21] |
| 370 | 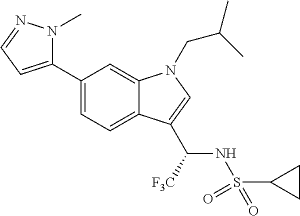 [SMILES: CC(C)Cn1cc([C@H](NS(=O)(=O)C2CC2)C(F)(F)F)c2ccc(-c3ccnn3C)cc21] |
| 371 | 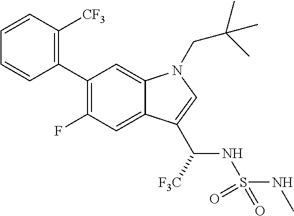 [SMILES: CNS(=O)(=O)N[C@@H](c1cn(CC(C)(C)C)c2cc(-c3ccccc3C(F)(F)F)c(F)cc12)C(F)(F)F] |
| 372 | 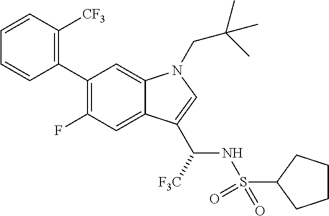 [SMILES: CC(C)(C)Cn1cc([C@H](NS(=O)(=O)C2CCCC2)C(F)(F)F)c2cc(F)c(-c3ccccc3C(F)(F)F)cc21] |
| 373 | 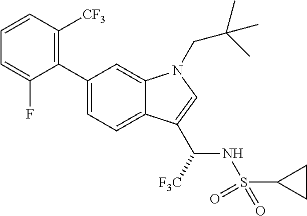 [SMILES: CC(C)(C)Cn1cc([C@H](NS(=O)(=O)C2CC2)C(F)(F)F)c2ccc(-c3c(F)cccc3C(F)(F)F)cc21] |
| 374 | 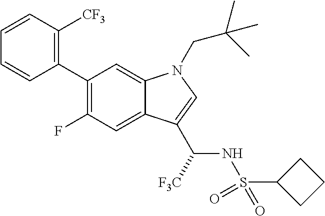 [SMILES: CC(C)(C)Cn1cc([C@H](NS(=O)(=O)C2CCC2)C(F)(F)F)c2cc(F)c(-c3ccccc3C(F)(F)F)cc21] |
| 375 | 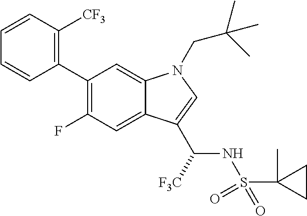 [SMILES: CC(C)(C)Cn1cc([C@H](NS(=O)(=O)C2(C)CC2)C(F)(F)F)c2cc(F)c(-c3ccccc3C(F)(F)F)cc21] |
| 376 | 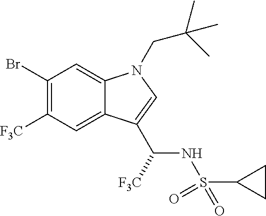 [SMILES: CC(C)(C)Cn1cc([C@H](NS(=O)(=O)C2CC2)C(F)(F)F)c2cc(C(F)(F)F)c(Br)cc21] |
| 377 | 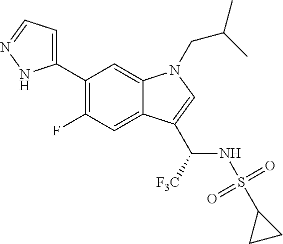 [SMILES: CC(C)Cn1cc([C@H](NS(=O)(=O)C2CC2)C(F)(F)F)c2cc(F)c(-c3ccn[nH]3)cc21] |
| 378 | 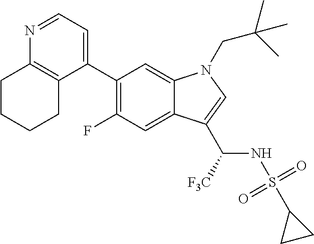 [SMILES: CC(C)(C)Cn1cc([C@H](NS(=O)(=O)C2CC2)C(F)(F)F)c2cc(F)c(-c3ccnc4c3CCCC4)cc21] |
| 379 | 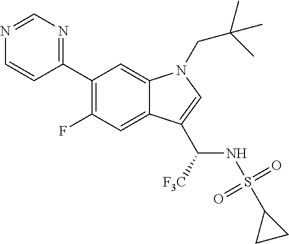 [SMILES: CC(C)(C)Cn1cc([C@H](NS(=O)(=O)C2CC2)C(F)(F)F)c2cc(F)c(-c3ccncn3)cc21] |
| 380 | 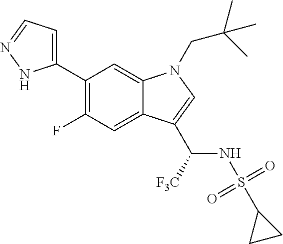 [SMILES: CC(C)(C)Cn1cc([C@H](NS(=O)(=O)C2CC2)C(F)(F)F)c2cc(F)c(-c3ccn[nH]3)cc21] |
| 381 | 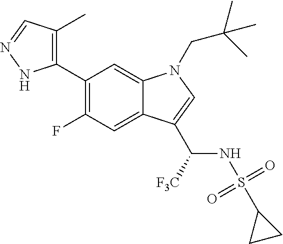 [SMILES: Cc1cn[nH]c1-c1cc2c(cc1F)c([C@H](NS(=O)(=O)C1CC1)C(F)(F)F)cn2CC(C)(C)C] |
| 382 | 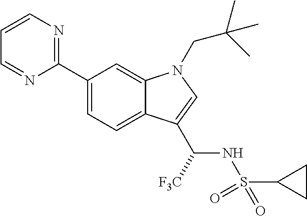 [SMILES: CC(C)(C)Cn1cc([C@H](NS(=O)(=O)C2CC2)C(F)(F)F)c2ccc(-c3ncccn3)cc21] |
| 383 | 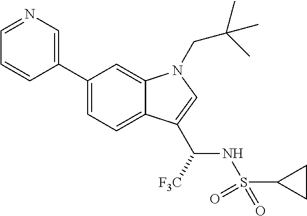 [SMILES: CC(C)(C)Cn1cc([C@H](NS(=O)(=O)C2CC2)C(F)(F)F)c2ccc(-c3cccnc3)cc21] |
| 384 | 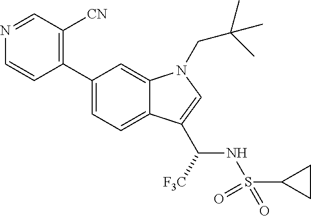 [SMILES: CC(C)(C)Cn1cc([C@H](NS(=O)(=O)C2CC2)C(F)(F)F)c2ccc(-c3ccncc3C#N)cc21] |
| 385 | 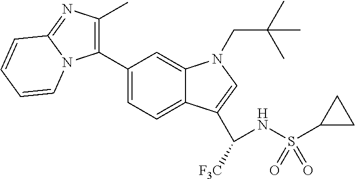 [SMILES: Cc1nc2ccccn2c1-c1ccc2c([C@H](NS(=O)(=O)C3CC3)C(F)(F)F)cn(CC(C)(C)C)c2c1] |
| 386 | 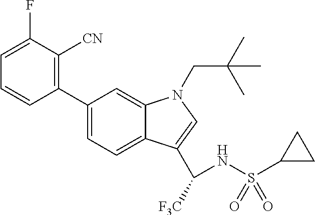 [SMILES: CC(C)(C)Cn1cc([C@H](NS(=O)(=O)C2CC2)C(F)(F)F)c2ccc(-c3cccc(F)c3C#N)cc21] |
| 387 | 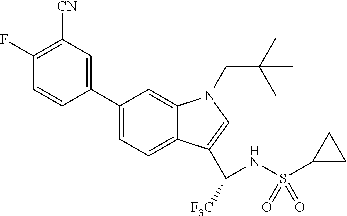 [SMILES: CC(C)(C)Cn1cc([C@H](NS(=O)(=O)C2CC2)C(F)(F)F)c2ccc(-c3ccc(F)c(C#N)c3)cc21] |
| 388 | 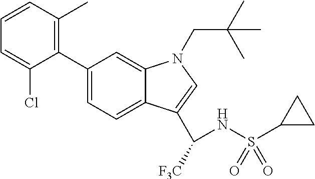 [SMILES: Cc1cccc(Cl)c1-c1ccc2c([C@H](NS(=O)(=O)C3CC3)C(F)(F)F)cn(CC(C)(C)C)c2c1] |
| 389 | 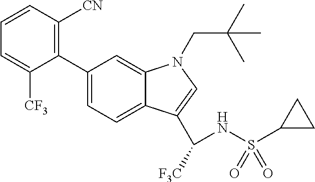 [SMILES: CC(C)(C)Cn1cc([C@H](NS(=O)(=O)C2CC2)C(F)(F)F)c2ccc(-c3c(C#N)cccc3C(F)(F)F)cc21] |
| 390 | 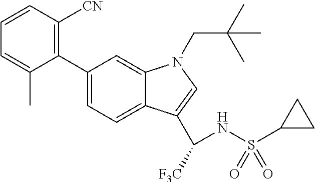 [SMILES: Cc1cccc(C#N)c1-c1ccc2c([C@H](NS(=O)(=O)C3CC3)C(F)(F)F)cn(CC(C)(C)C)c2c1] |
| 391 | 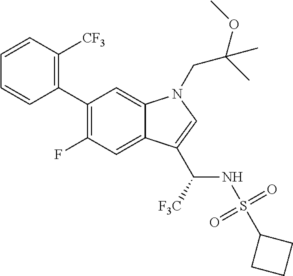 [SMILES: COC(C)(C)Cn1cc([C@H](NS(=O)(=O)C2CCC2)C(F)(F)F)c2cc(F)c(-c3ccccc3C(F)(F)F)cc21] |
| 392 | 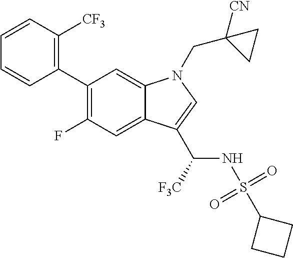 [SMILES: N#CC1(Cn2cc([C@H](NS(=O)(=O)C3CCC3)C(F)(F)F)c3cc(F)c(-c4ccccc4C(F)(F)F)cc32)CC1] |
| 393 | 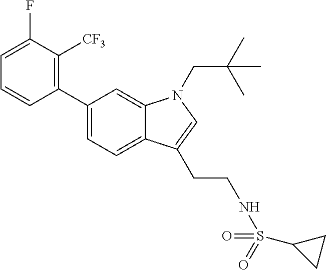 [SMILES: CC(C)(C)Cn1cc(CCNS(=O)(=O)C2CC2)c2ccc(-c3cccc(F)c3C(F)(F)F)cc21] |
| 394 | 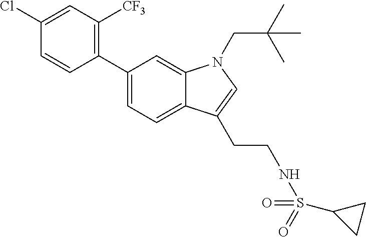 [SMILES: CC(C)(C)Cn1cc(CCNS(=O)(=O)C2CC2)c2ccc(-c3ccc(Cl)cc3C(F)(F)F)cc21] |
| 395 | 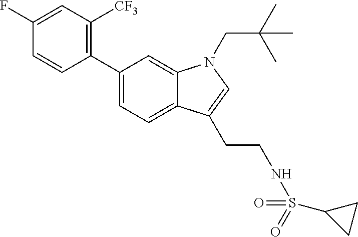 [SMILES: CC(C)(C)Cn1cc(CCNS(=O)(=O)C2CC2)c2ccc(-c3ccc(F)cc3C(F)(F)F)cc21] |
| 396 | 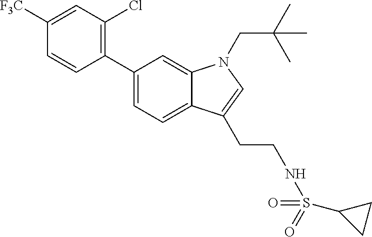 [SMILES: CC(C)(C)Cn1cc(CCNS(=O)(=O)C2CC2)c2ccc(-c3ccc(C(F)(F)F)cc3Cl)cc21] |
| 397 | 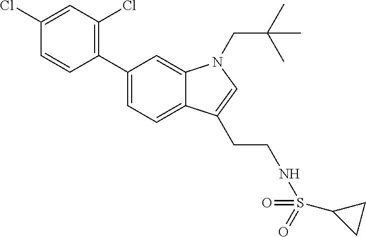 [SMILES: CC(C)(C)Cn1cc(CCNS(=O)(=O)C2CC2)c2ccc(-c3ccc(Cl)cc3Cl)cc21] |
| 398 | 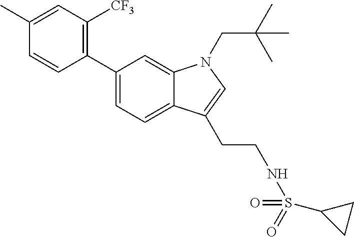 [SMILES: Cc1ccc(-c2ccc3c(CCNS(=O)(=O)C4CC4)cn(CC(C)(C)C)c3c2)c(C(F)(F)F)c1] |
| 399 | 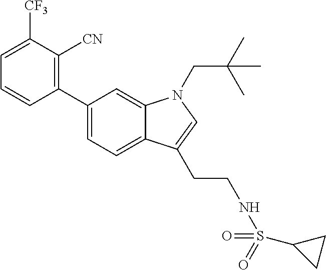 [SMILES: CC(C)(C)Cn1cc(CCNS(=O)(=O)C2CC2)c2ccc(-c3cccc(C(F)(F)F)c3C#N)cc21] |
| 400 | 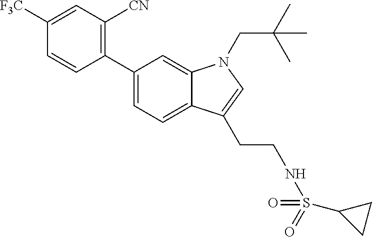 [SMILES: CC(C)(C)Cn1cc(CCNS(=O)(=O)C2CC2)c2ccc(-c3ccc(C(F)(F)F)cc3C#N)cc21] |
| 401 | 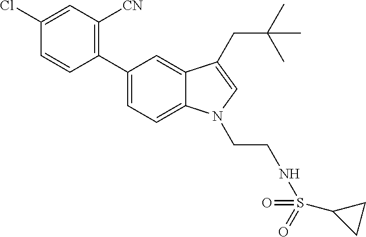 [SMILES: CC(C)(C)Cc1cn(CCNS(=O)(=O)C2CC2)c2ccc(-c3ccc(Cl)cc3C#N)cc12] |
| 402 | 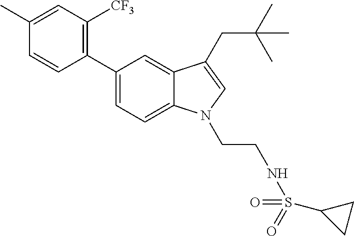 [SMILES: Cc1ccc(-c2ccc3c(c2)c(CC(C)(C)C)cn3CCNS(=O)(=O)C2CC2)c(C(F)(F)F)c1] |
| 403 | 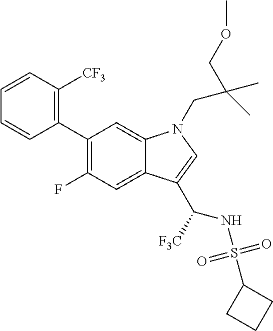 [SMILES: COCC(C)(C)Cn1cc([C@H](NS(=O)(=O)C2CCC2)C(F)(F)F)c2cc(F)c(-c3ccccc3C(F)(F)F)cc21] |
| 404 | 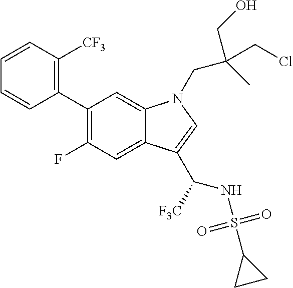 [SMILES: CC(CO)(CCl)Cn1cc([C@H](NS(=O)(=O)C2CC2)C(F)(F)F)c2cc(F)c(-c3ccccc3C(F)(F)F)cc21] |
| 405 | 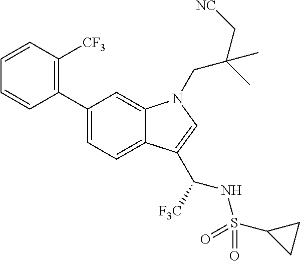 [SMILES: CC(C)(CC#N)Cn1cc([C@H](NS(=O)(=O)C2CC2)C(F)(F)F)c2ccc(-c3ccccc3C(F)(F)F)cc21] |
| 406 | 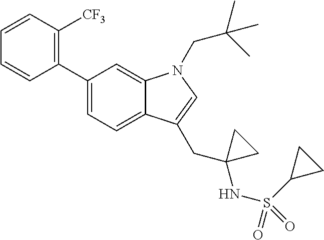 [SMILES: CC(C)(C)Cn1cc(CC2(NS(=O)(=O)C3CC3)CC2)c2ccc(-c3ccccc3C(F)(F)F)cc21] |
| 407 | 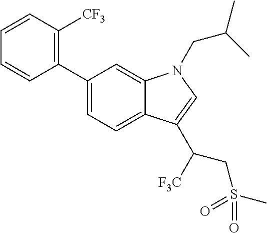 [SMILES: CC(C)Cn1cc(C(CS(C)(=O)=O)C(F)(F)F)c2ccc(-c3ccccc3C(F)(F)F)cc21] |
| 408 | 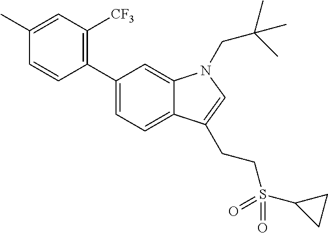 [SMILES: Cc1ccc(-c2ccc3c(CCS(=O)(=O)C4CC4)cn(CC(C)(C)C)c3c2)c(C(F)(F)F)c1] |
| 409 | 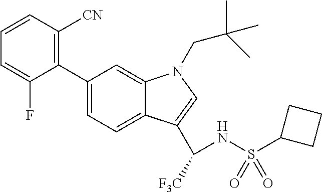 [SMILES: CC(C)(C)Cn1cc([C@H](NS(=O)(=O)C2CCC2)C(F)(F)F)c2ccc(-c3c(F)cccc3C#N)cc21] |
| 410 | 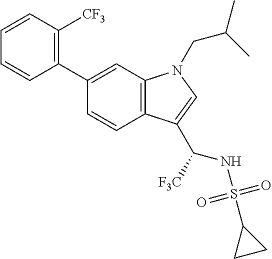 [SMILES: CC(C)Cn1cc([C@H](NS(=O)(=O)C2CC2)C(F)(F)F)c2ccc(-c3ccccc3C(F)(F)F)cc21] |
| 411 | 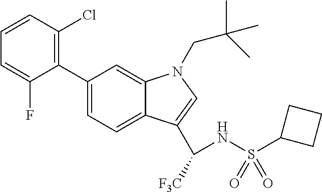 [SMILES: CC(C)(C)Cn1cc([C@H](NS(=O)(=O)C2CCC2)C(F)(F)F)c2ccc(-c3c(F)cccc3Cl)cc21] |
| 412 | 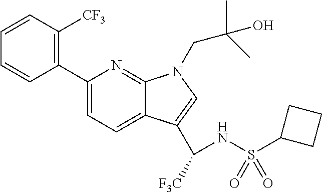 [SMILES: CC(C)(O)Cn1cc([C@H](NS(=O)(=O)C2CCC2)C(F)(F)F)c2ccc(-c3ccccc3C(F)(F)F)nc21] |
| 413 | 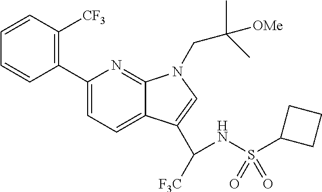 [SMILES: COC(C)(C)Cn1cc(C(NS(=O)(=O)C2CCC2)C(F)(F)F)c2ccc(-c3ccccc3C(F)(F)F)nc21] |
| 414 | 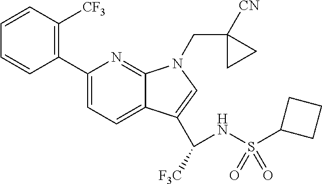 [SMILES: N#CC1(Cn2cc([C@H](NS(=O)(=O)C3CCC3)C(F)(F)F)c3ccc(-c4ccccc4C(F)(F)F)nc32)CC1] |
| 415 | 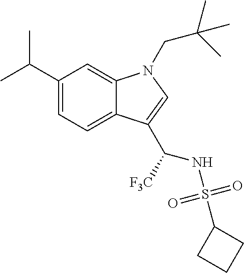 [SMILES: CC(C)c1ccc2c([C@H](NS(=O)(=O)C3CCC3)C(F)(F)F)cn(CC(C)(C)C)c2c1] |
| 416 | 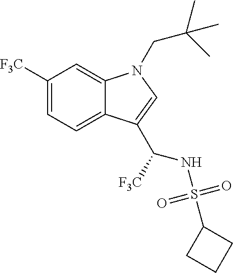 [SMILES: CC(C)(C)Cn1cc([C@H](NS(=O)(=O)C2CCC2)C(F)(F)F)c2ccc(C(F)(F)F)cc21] |
| 417 | 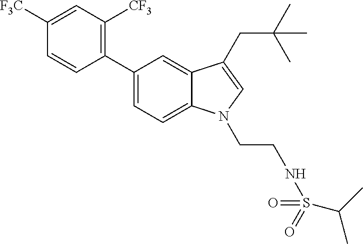 [SMILES: CC(C)S(=O)(=O)NCCn1cc(CC(C)(C)C)c2cc(-c3ccc(C(F)(F)F)cc3C(F)(F)F)ccc21] |
| 418 | 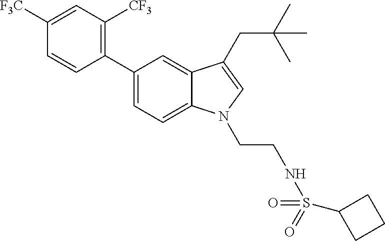 [SMILES: CC(C)(C)Cc1cn(CCNS(=O)(=O)C2CCC2)c2ccc(-c3ccc(C(F)(F)F)cc3C(F)(F)F)cc12] |
| 419 | 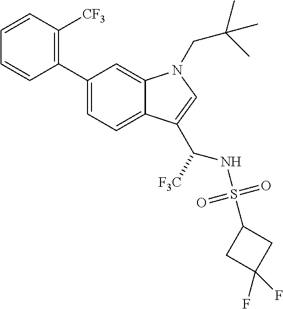 [SMILES: CC(C)(C)Cn1cc([C@H](NS(=O)(=O)C2CC(F)(F)C2)C(F)(F)F)c2ccc(-c3ccccc3C(F)(F)F)cc21] |
| 420 | 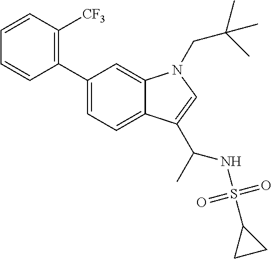 [SMILES: CC(NS(=O)(=O)C1CC1)c1cn(CC(C)(C)C)c2cc(-c3ccccc3C(F)(F)F)ccc12] |
| 421 | 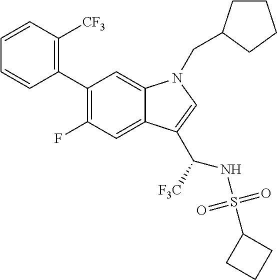 [SMILES: O=S(=O)(N[C@@H](c1cn(CC2CCCC2)c2cc(-c3ccccc3C(F)(F)F)c(F)cc12)C(F)(F)F)C1CCC1] |
| 422 | 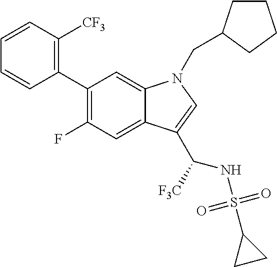 [SMILES: O=S(=O)(N[C@@H](c1cn(CC2CCCC2)c2cc(-c3ccccc3C(F)(F)F)c(F)cc12)C(F)(F)F)C1CC1] |
| 423 | 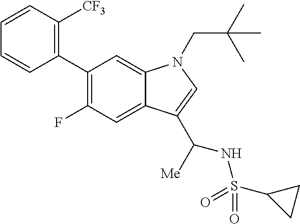 [SMILES: CC(NS(=O)(=O)C1CC1)c1cn(CC(C)(C)C)c2cc(-c3ccccc3C(F)(F)F)c(F)cc12] |
| 424 | 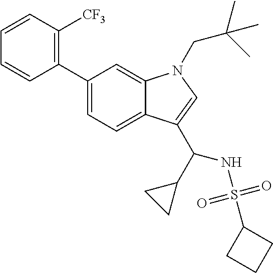 [SMILES: CC(C)(C)Cn1cc(C(NS(=O)(=O)C2CCC2)C2CC2)c2ccc(-c3ccccc3C(F)(F)F)cc21] |
| 425 | 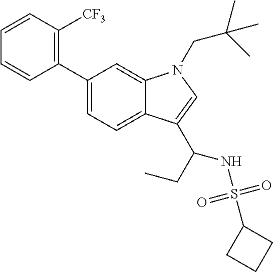 [SMILES: CCC(NS(=O)(=O)C1CCC1)c1cn(CC(C)(C)C)c2cc(-c3ccccc3C(F)(F)F)ccc12] |
| 426 | 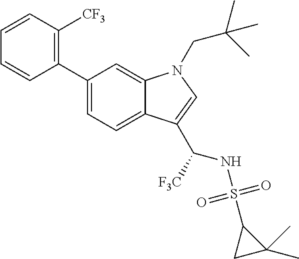 [SMILES: CC(C)(C)Cn1cc([C@H](NS(=O)(=O)C2CC2(C)C)C(F)(F)F)c2ccc(-c3ccccc3C(F)(F)F)cc21] |
| 427 | 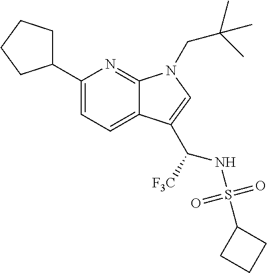 [SMILES: CC(C)(C)Cn1cc([C@H](NS(=O)(=O)C2CCC2)C(F)(F)F)c2ccc(C3CCCC3)nc21] |
| 428 | 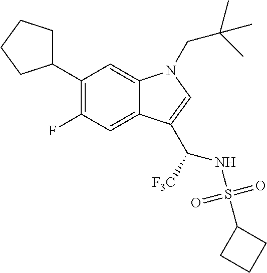 [SMILES: CC(C)(C)Cn1cc([C@H](NS(=O)(=O)C2CCC2)C(F)(F)F)c2cc(F)c(C3CCCC3)cc21] |
| 429 | 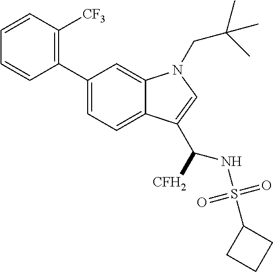 |
| 430 | 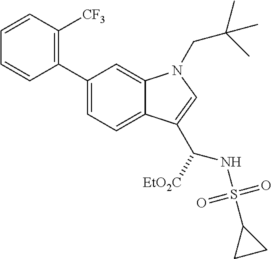 [SMILES: CCOC(=O)[C@@H](NS(=O)(=O)C1CC1)c1cn(CC(C)(C)C)c2cc(-c3ccccc3C(F)(F)F)ccc12] |
| 431 | 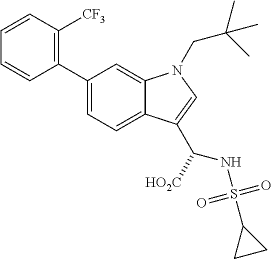 [SMILES: CC(C)(C)Cn1cc([C@H](NS(=O)(=O)C2CC2)C(=O)O)c2ccc(-c3ccccc3C(F)(F)F)cc21] |
| 432 | 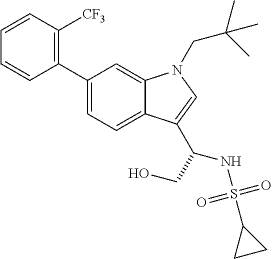 [SMILES: CC(C)(C)Cn1cc([C@@H](CO)NS(=O)(=O)C2CC2)c2ccc(-c3ccccc3C(F)(F)F)cc21] |
| 433 | 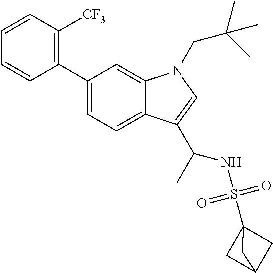 [SMILES: CC(NS(=O)(=O)C12CC(C1)C2)c1cn(CC(C)(C)C)c2cc(-c3ccccc3C(F)(F)F)ccc12] |
| 434 | 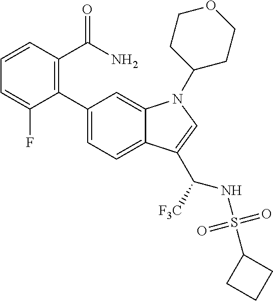 [SMILES: NC(=O)c1cccc(F)c1-c1ccc2c([C@H](NS(=O)(=O)C3CCC3)C(F)(F)F)cn(C3CCOCC3)c2c1] |
| 435 | 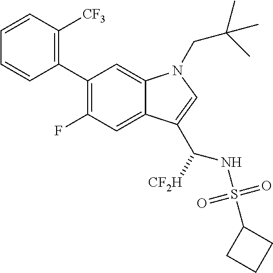 [SMILES: CC(C)(C)Cn1cc([C@H](NS(=O)(=O)C2CCC2)C(F)F)c2cc(F)c(-c3ccccc3C(F)(F)F)cc21] |
| 436 | 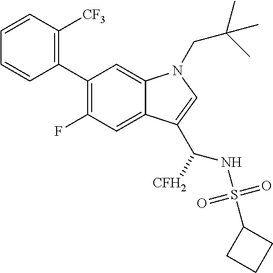 |
| 437 | 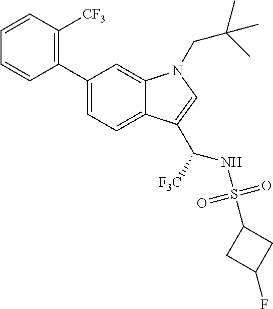 [SMILES: CC(C)(C)Cn1cc([C@H](NS(=O)(=O)C2CC(F)C2)C(F)(F)F)c2ccc(-c3ccccc3C(F)(F)F)cc21] |
| 438 | 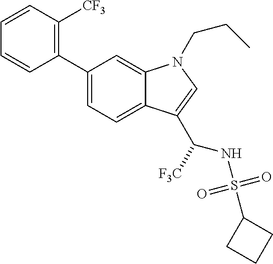 [SMILES: CCCn1cc([C@H](NS(=O)(=O)C2CCC2)C(F)(F)F)c2ccc(-c3ccccc3C(F)(F)F)cc21] |
| 439 | 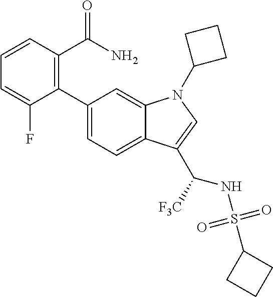 [SMILES: NC(=O)c1cccc(F)c1-c1ccc2c([C@H](NS(=O)(=O)C3CCC3)C(F)(F)F)cn(C3CCC3)c2c1] |
| 440 | 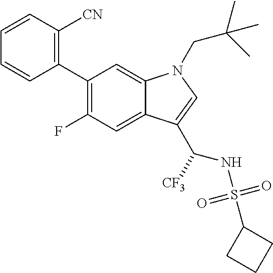 [SMILES: CC(C)(C)Cn1cc([C@H](NS(=O)(=O)C2CCC2)C(F)(F)F)c2cc(F)c(-c3ccccc3C#N)cc21] |
| 441 | 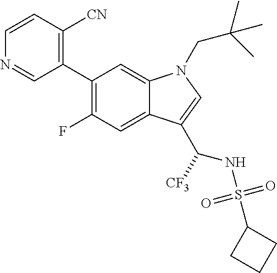 [SMILES: CC(C)(C)Cn1cc([C@H](NS(=O)(=O)C2CCC2)C(F)(F)F)c2cc(F)c(-c3cnccc3C#N)cc21] |
| 442 | 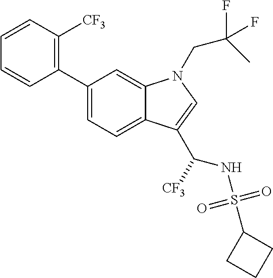 [SMILES: CC(F)(F)Cn1cc([C@H](NS(=O)(=O)C2CCC2)C(F)(F)F)c2ccc(-c3ccccc3C(F)(F)F)cc21] |
| 444 | 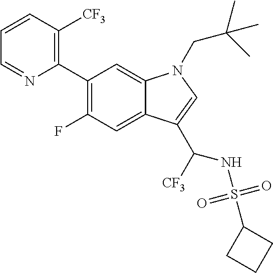 [SMILES: CC(C)(C)Cn1cc(C(NS(=O)(=O)C2CCC2)C(F)(F)F)c2cc(F)c(-c3ncccc3C(F)(F)F)cc21] |
| 445 | 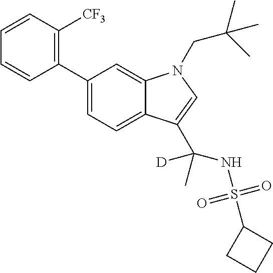 [SMILES: [2H]C(C)(NS(=O)(=O)C1CCC1)c1cn(CC(C)(C)C)c2cc(-c3ccccc3C(F)(F)F)ccc12] |
| 446 | 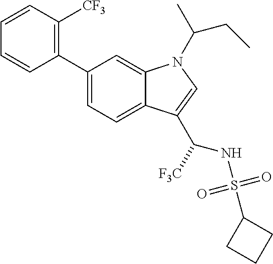 [SMILES: CCC(C)n1cc([C@H](NS(=O)(=O)C2CCC2)C(F)(F)F)c2ccc(-c3ccccc3C(F)(F)F)cc21] |
| 454 | 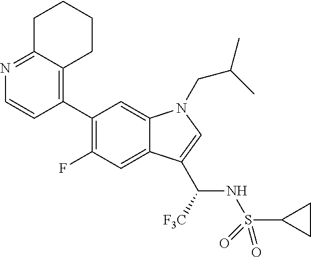 [SMILES: CC(C)Cn1cc([C@H](NS(=O)(=O)C2CC2)C(F)(F)F)c2cc(F)c(-c3ccnc4c3CCCC4)cc21] |
| 455 | 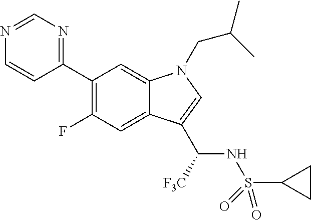 [SMILES: CC(C)Cn1cc([C@H](NS(=O)(=O)C2CC2)C(F)(F)F)c2cc(F)c(-c3ccncn3)cc21] |
| 456 | 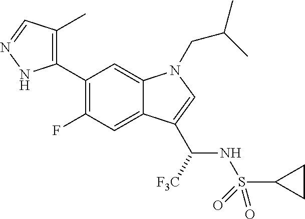 [SMILES: Cc1cn[nH]c1-c1cc2c(cc1F)c([C@H](NS(=O)(=O)C1CC1)C(F)(F)F)cn2CC(C)C] |
| 457 | 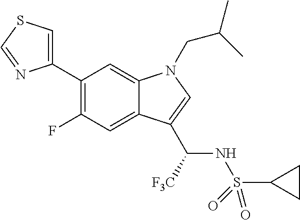 [SMILES: CC(C)Cn1cc([C@H](NS(=O)(=O)C2CC2)C(F)(F)F)c2cc(F)c(-c3cscn3)cc21] |
| 458 | 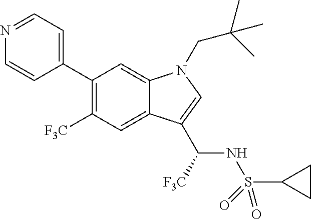 [SMILES: CC(C)(C)Cn1cc([C@H](NS(=O)(=O)C2CC2)C(F)(F)F)c2cc(C(F)(F)F)c(-c3ccncc3)cc21] |
| 459 | 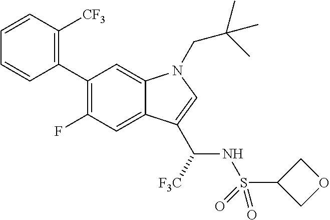 [SMILES: CC(C)(C)Cn1cc([C@H](NS(=O)(=O)C2COC2)C(F)(F)F)c2cc(F)c(-c3ccccc3C(F)(F)F)cc21] |
| 460 | 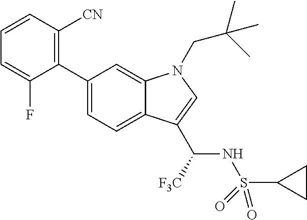 [SMILES: CC(C)(C)Cn1cc([C@H](NS(=O)(=O)C2CC2)C(F)(F)F)c2ccc(-c3c(F)cccc3C#N)cc21] |
| 461 | 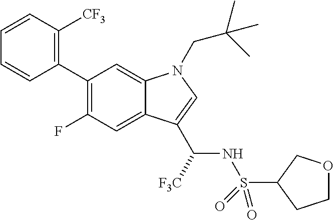 [SMILES: CC(C)(C)Cn1cc([C@H](NS(=O)(=O)C2CCOC2)C(F)(F)F)c2cc(F)c(-c3ccccc3C(F)(F)F)cc21] |
| 462 | 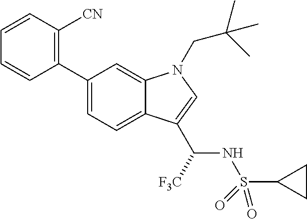 [SMILES: CC(C)(C)Cn1cc([C@H](NS(=O)(=O)C2CC2)C(F)(F)F)c2ccc(-c3ccccc3C#N)cc21] |
| 463 | 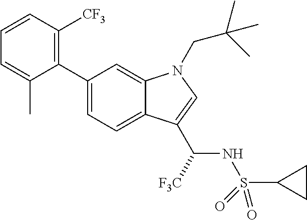 [SMILES: Cc1cccc(C(F)(F)F)c1-c1ccc2c([C@H](NS(=O)(=O)C3CC3)C(F)(F)F)cn(CC(C)(C)C)c2c1] |
| 464 | 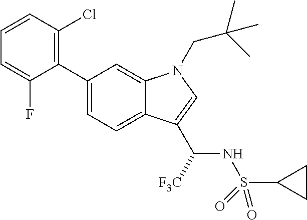 [SMILES: CC(C)(C)Cn1cc([C@H](NS(=O)(=O)C2CC2)C(F)(F)F)c2ccc(-c3c(F)cccc3Cl)cc21] |
| 465 | 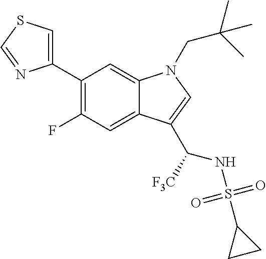 [SMILES: CC(C)(C)Cn1cc([C@H](NS(=O)(=O)C2CC2)C(F)(F)F)c2cc(F)c(-c3cscn3)cc21] |
| 466 | 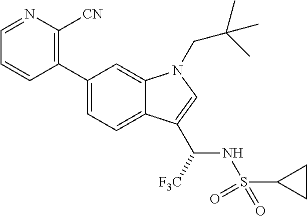 [SMILES: CC(C)(C)Cn1cc([C@H](NS(=O)(=O)C2CC2)C(F)(F)F)c2ccc(-c3cccnc3C#N)cc21] |
| 467 | 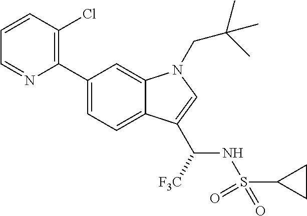 [SMILES: CC(C)(C)Cn1cc([C@H](NS(=O)(=O)C2CC2)C(F)(F)F)c2ccc(-c3ncccc3Cl)cc21] |
| 468 | 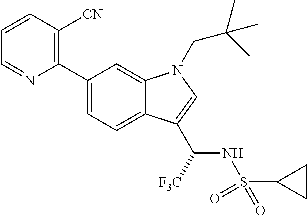 [SMILES: CC(C)(C)Cn1cc([C@H](NS(=O)(=O)C2CC2)C(F)(F)F)c2ccc(-c3ncccc3C#N)cc21] |
| 469 | 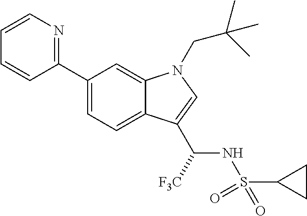 [SMILES: CC(C)(C)Cn1cc([C@H](NS(=O)(=O)C2CC2)C(F)(F)F)c2ccc(-c3ccccn3)cc21] |
| 470 | 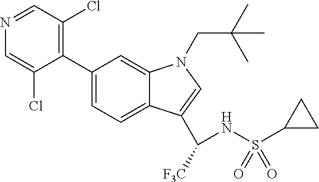 [SMILES: CC(C)(C)Cn1cc([C@H](NS(=O)(=O)C2CC2)C(F)(F)F)c2ccc(-c3c(Cl)cncc3Cl)cc21] |
| 471 | 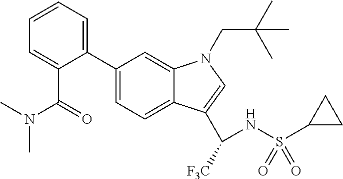 [SMILES: CN(C)C(=O)c1ccccc1-c1ccc2c([C@H](NS(=O)(=O)C3CC3)C(F)(F)F)cn(CC(C)(C)C)c2c1] |
| 472 | 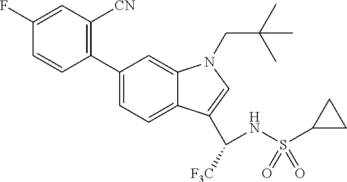 [SMILES: CC(C)(C)Cn1cc([C@H](NS(=O)(=O)C2CC2)C(F)(F)F)c2ccc(-c3ccc(F)cc3C#N)cc21] |
| 473 | 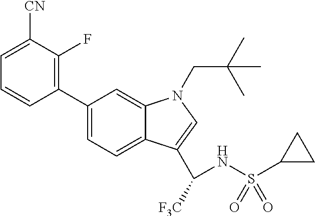 [SMILES: CC(C)(C)Cn1cc([C@H](NS(=O)(=O)C2CC2)C(F)(F)F)c2ccc(-c3cccc(C#N)c3F)cc21] |
| 474 | 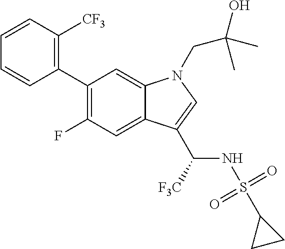 [SMILES: CC(C)(O)Cn1cc([C@H](NS(=O)(=O)C2CC2)C(F)(F)F)c2cc(F)c(-c3ccccc3C(F)(F)F)cc21] |
| 475 | 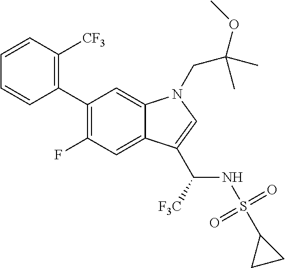 [SMILES: COC(C)(C)Cn1cc([C@H](NS(=O)(=O)C2CC2)C(F)(F)F)c2cc(F)c(-c3ccccc3C(F)(F)F)cc21] |
| 476 | 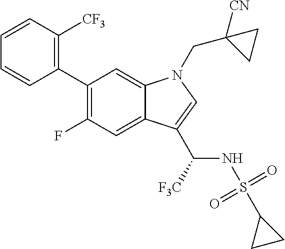 [SMILES: N#CC1(Cn2cc([C@H](NS(=O)(=O)C3CC3)C(F)(F)F)c3cc(F)c(-c4ccccc4C(F)(F)F)cc32)CC1] |
| 477 | 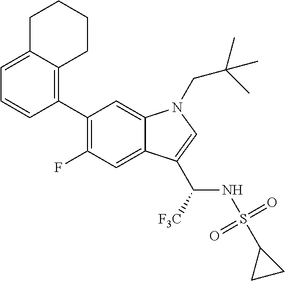 [SMILES: CC(C)(C)Cn1cc([C@H](NS(=O)(=O)C2CC2)C(F)(F)F)c2cc(F)c(-c3cccc4c3CCCC4)cc21] |
| 478 | 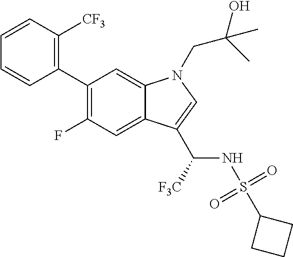 [SMILES: CC(C)(O)Cn1cc([C@H](NS(=O)(=O)C2CCC2)C(F)(F)F)c2cc(F)c(-c3ccccc3C(F)(F)F)cc21] |
| 479 | 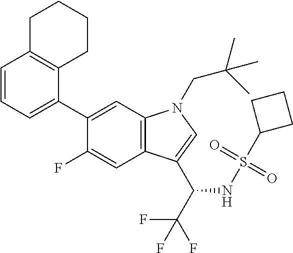 [SMILES: CC(C)(C)Cn1cc([C@H](NS(=O)(=O)C2CCC2)C(F)(F)F)c2cc(F)c(-c3cccc4c3CCCC4)cc21] |
| 480 | 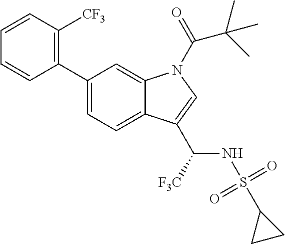 [SMILES: CC(C)(C)C(=O)n1cc([C@H](NS(=O)(=O)C2CC2)C(F)(F)F)c2ccc(-c3ccccc3C(F)(F)F)cc21] |
| 481 | 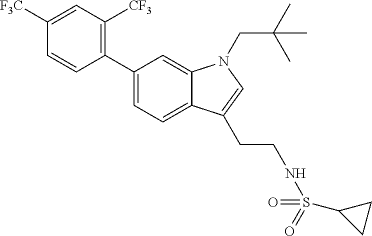 [SMILES: CC(C)(C)Cn1cc(CCNS(=O)(=O)C2CC2)c2ccc(-c3ccc(C(F)(F)F)cc3C(F)(F)F)cc21] |
| 482 | 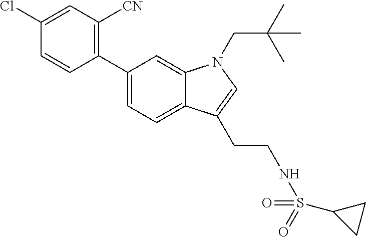 [SMILES: CC(C)(C)Cn1cc(CCNS(=O)(=O)C2CC2)c2ccc(-c3ccc(Cl)cc3C#N)cc21] |
| 483 | 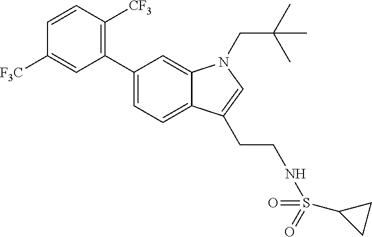 [SMILES: CC(C)(C)Cn1cc(CCNS(=O)(=O)C2CC2)c2ccc(-c3cc(C(F)(F)F)ccc3C(F)(F)F)cc21] |
| 484 | 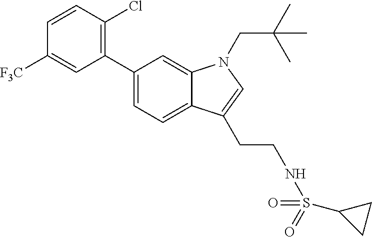 [SMILES: CC(C)(C)Cn1cc(CCNS(=O)(=O)C2CC2)c2ccc(-c3cc(C(F)(F)F)ccc3Cl)cc21] |
| 485 | 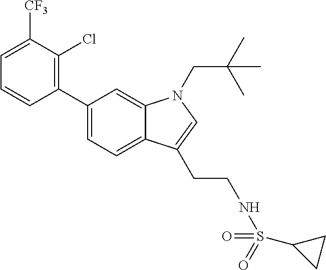 [SMILES: CC(C)(C)Cn1cc(CCNS(=O)(=O)C2CC2)c2ccc(-c3cccc(C(F)(F)F)c3Cl)cc21] |
| 486 | 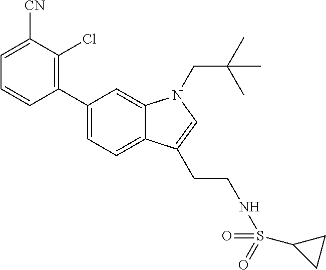 [SMILES: CC(C)(C)Cn1cc(CCNS(=O)(=O)C2CC2)c2ccc(-c3cccc(C#N)c3Cl)cc21] |
| 487 | 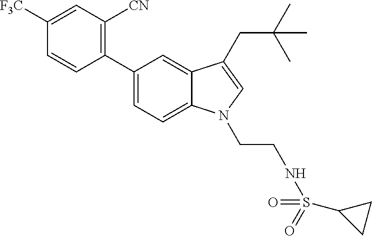 [SMILES: CC(C)(C)Cc1cn(CCNS(=O)(=O)C2CC2)c2ccc(-c3ccc(C(F)(F)F)cc3C#N)cc12] |
| 488 | 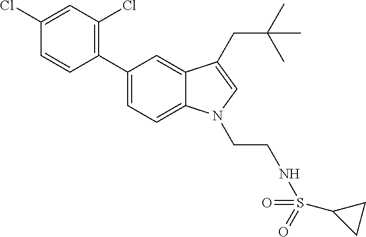 [SMILES: CC(C)(C)Cc1cn(CCNS(=O)(=O)C2CC2)c2ccc(-c3ccc(Cl)cc3Cl)cc12] |
| 489 | 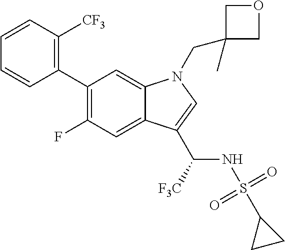 [SMILES: CC1(Cn2cc([C@H](NS(=O)(=O)C3CC3)C(F)(F)F)c3cc(F)c(-c4ccccc4C(F)(F)F)cc32)COC1] |
| 490 | 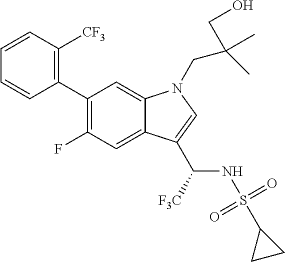 [SMILES: CC(C)(CO)Cn1cc([C@H](NS(=O)(=O)C2CC2)C(F)(F)F)c2cc(F)c(-c3ccccc3C(F)(F)F)cc21] |
| 491 | 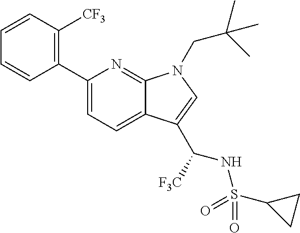 [SMILES: CC(C)(C)Cn1cc([C@H](NS(=O)(=O)C2CC2)C(F)(F)F)c2ccc(-c3ccccc3C(F)(F)F)nc21] |
| 492 | 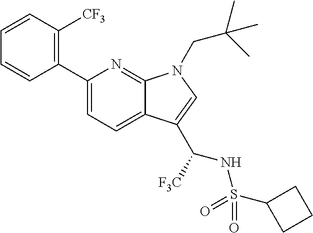 [SMILES: CC(C)(C)Cn1cc([C@H](NS(=O)(=O)C2CCC2)C(F)(F)F)c2ccc(-c3ccccc3C(F)(F)F)nc21] |
| 493 | 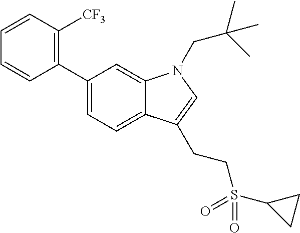 [SMILES: CC(C)(C)Cn1cc(CCS(=O)(=O)C2CC2)c2ccc(-c3ccccc3C(F)(F)F)cc21] |
| 494 | 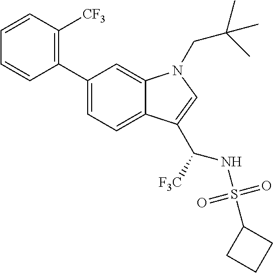 [SMILES: CC(C)(C)Cn1cc([C@H](NS(=O)(=O)C2CCC2)C(F)(F)F)c2ccc(-c3ccccc3C(F)(F)F)cc21] |
| 495 | 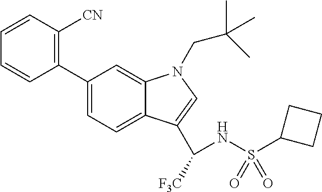 [SMILES: CC(C)(C)Cn1cc([C@H](NS(=O)(=O)C2CCC2)C(F)(F)F)c2ccc(-c3ccccc3C#N)cc21] |
| 496 | 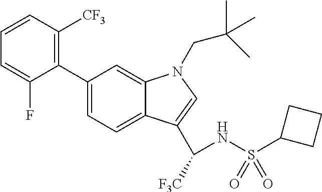 [SMILES: CC(C)(C)Cn1cc([C@H](NS(=O)(=O)C2CCC2)C(F)(F)F)c2ccc(-c3c(F)cccc3C(F)(F)F)cc21] |
| 497 | 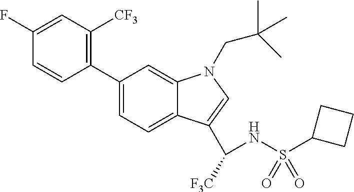 [SMILES: CC(C)(C)Cn1cc([C@H](NS(=O)(=O)C2CCC2)C(F)(F)F)c2ccc(-c3ccc(F)cc3C(F)(F)F)cc21] |
| 498 | 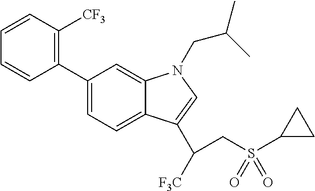 [SMILES: CC(C)Cn1cc(C(CS(=O)(=O)C2CC2)C(F)(F)F)c2ccc(-c3ccccc3C(F)(F)F)cc21] |
| 499 | 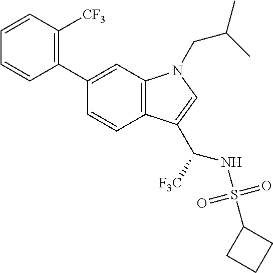 [SMILES: CC(C)Cn1cc([C@H](NS(=O)(=O)C2CCC2)C(F)(F)F)c2ccc(-c3ccccc3C(F)(F)F)cc21] |
| 500 | 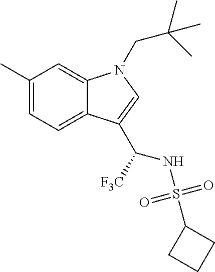 [SMILES: Cc1ccc2c([C@H](NS(=O)(=O)C3CCC3)C(F)(F)F)cn(CC(C)(C)C)c2c1] |
| 501 | 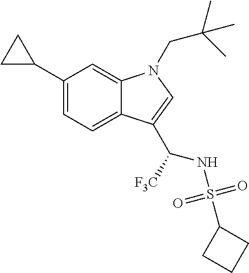 [SMILES: CC(C)(C)Cn1cc([C@H](NS(=O)(=O)C2CCC2)C(F)(F)F)c2ccc(C3CC3)cc21] |
| 502 | 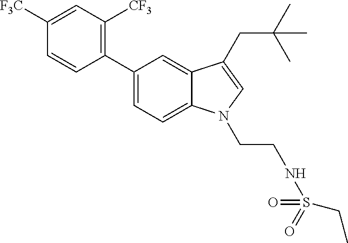 [SMILES: CCS(=O)(=O)NCCn1cc(CC(C)(C)C)c2cc(-c3ccc(C(F)(F)F)cc3C(F)(F)F)ccc21] |
| 503 | 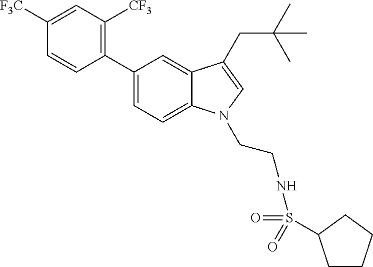 [SMILES: CC(C)(C)Cc1cn(CCNS(=O)(=O)C2CCCC2)c2ccc(-c3ccc(C(F)(F)F)cc3C(F)(F)F)cc12] |
| 504 | 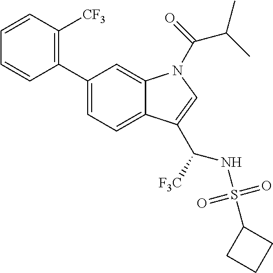 [SMILES: CC(C)C(=O)n1cc([C@H](NS(=O)(=O)C2CCC2)C(F)(F)F)c2ccc(-c3ccccc3C(F)(F)F)cc21] |
| 505 | 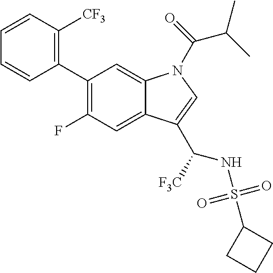 [SMILES: CC(C)C(=O)n1cc([C@H](NS(=O)(=O)C2CCC2)C(F)(F)F)c2cc(F)c(-c3ccccc3C(F)(F)F)cc21] |
| 506 | 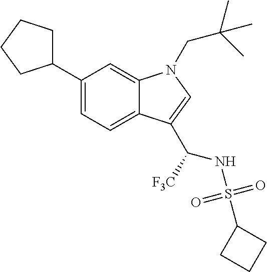 [SMILES: CC(C)(C)Cn1cc([C@H](NS(=O)(=O)C2CCC2)C(F)(F)F)c2ccc(C3CCCC3)cc21] |
| 507 | 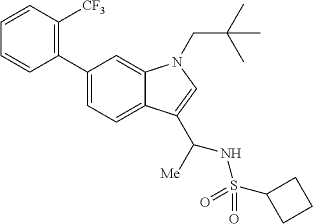 [SMILES: CC(NS(=O)(=O)C1CCC1)c1cn(CC(C)(C)C)c2cc(-c3ccccc3C(F)(F)F)ccc12] |
| 508 | 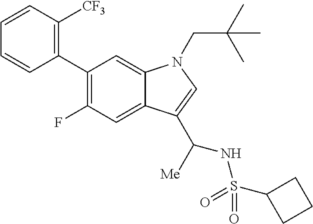 [SMILES: CC(NS(=O)(=O)C1CCC1)c1cn(CC(C)(C)C)c2cc(-c3ccccc3C(F)(F)F)c(F)cc12] |
| 509 | 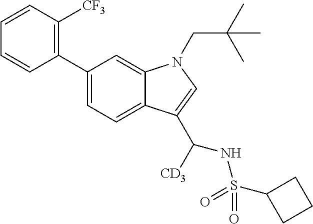 [SMILES: [2H]C([2H])([2H])C(NS(=O)(=O)C1CCC1)c1cn(CC(C)(C)C)c2cc(-c3ccccc3C(F)(F)F)ccc12] |
| 510 | 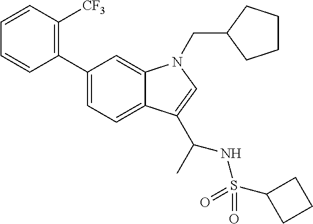 [SMILES: CC(NS(=O)(=O)C1CCC1)c1cn(CC2CCCC2)c2cc(-c3ccccc3C(F)(F)F)ccc12] |
| 511 | 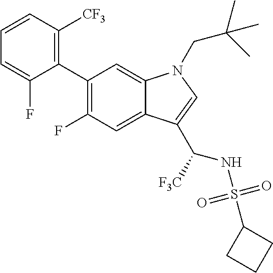 [SMILES: CC(C)(C)Cn1cc([C@H](NS(=O)(=O)C2CCC2)C(F)(F)F)c2cc(F)c(-c3c(F)cccc3C(F)(F)F)cc21] |
| 512 | 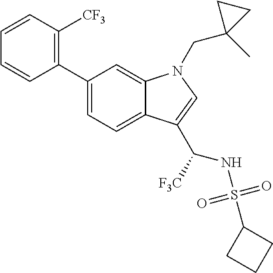 [SMILES: CC1(Cn2cc([C@H](NS(=O)(=O)C3CCC3)C(F)(F)F)c3ccc(-c4ccccc4C(F)(F)F)cc32)CC1] |
| 513 | 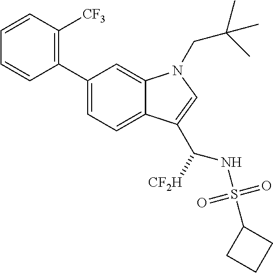 [SMILES: CC(C)(C)Cn1cc([C@H](NS(=O)(=O)C2CCC2)C(F)F)c2ccc(-c3ccccc3C(F)(F)F)cc21] |
| 514 | 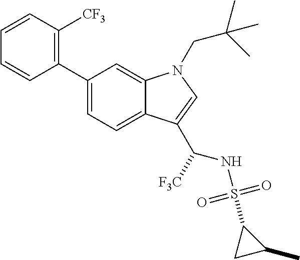 |
| 515 | 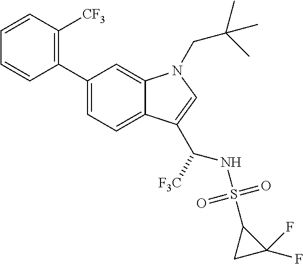 [SMILES: CC(C)(C)Cn1cc([C@H](NS(=O)(=O)C2CC2(F)F)C(F)(F)F)c2ccc(-c3ccccc3C(F)(F)F)cc21] |
| 516 | 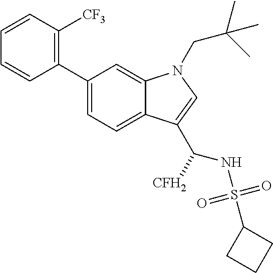 |
| 517 | 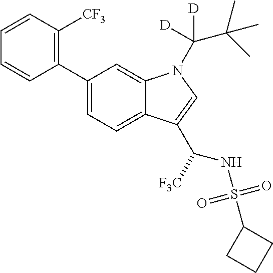 [SMILES: [2H]C([2H])(n1cc([C@H](NS(=O)(=O)C2CCC2)C(F)(F)F)c2ccc(-c3ccccc3C(F)(F)F)cc21)C(C)(C)C] |
| 518 | 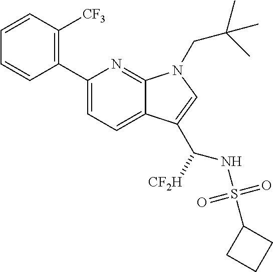 [SMILES: CC(C)(C)Cn1cc([C@H](NS(=O)(=O)C2CCC2)C(F)F)c2ccc(-c3ccccc3C(F)(F)F)nc21] |
| 519 | 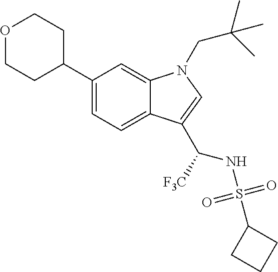 [SMILES: CC(C)(C)Cn1cc([C@H](NS(=O)(=O)C2CCC2)C(F)(F)F)c2ccc(C3CCOCC3)cc21] |
| 520 | 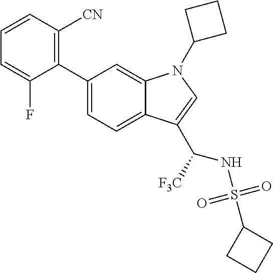 [SMILES: N#Cc1cccc(F)c1-c1ccc2c([C@H](NS(=O)(=O)C3CCC3)C(F)(F)F)cn(C3CCC3)c2c1] |
| 521 | 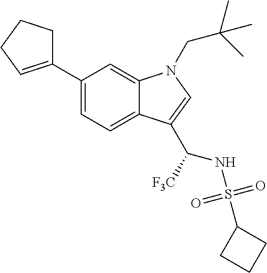 [SMILES: CC(C)(C)Cn1cc([C@H](NS(=O)(=O)C2CCC2)C(F)(F)F)c2ccc(C3=CCCC3)cc21] |
| 522 | 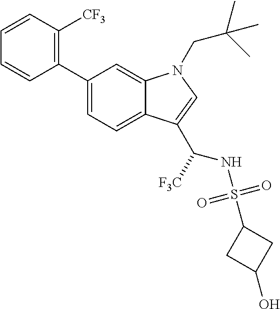 [SMILES: CC(C)(C)Cn1cc([C@H](NS(=O)(=O)C2CC(O)C2)C(F)(F)F)c2ccc(-c3ccccc3C(F)(F)F)cc21] |
| 523 | 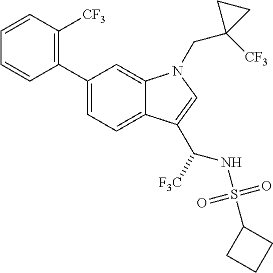 [SMILES: O=S(=O)(N[C@@H](c1cn(CC2(C(F)(F)F)CC2)c2cc(-c3ccccc3C(F)(F)F)ccc12)C(F)(F)F)C1CCC1] |
| 524 | 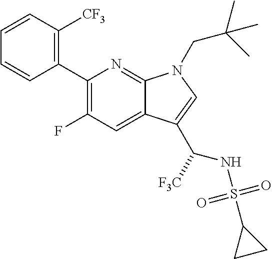 [SMILES: CC(C)(C)Cn1cc([C@H](NS(=O)(=O)C2CC2)C(F)(F)F)c2cc(F)c(-c3ccccc3C(F)(F)F)nc21] |
| 525 | 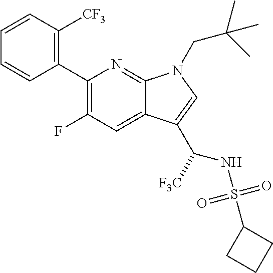 [SMILES: CC(C)(C)Cn1cc([C@H](NS(=O)(=O)C2CCC2)C(F)(F)F)c2cc(F)c(-c3ccccc3C(F)(F)F)nc21] |
| 526 | 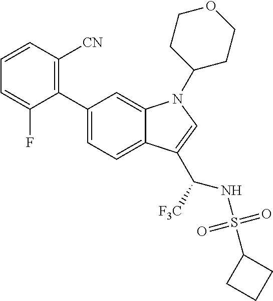 [SMILES: N#Cc1cccc(F)c1-c1ccc2c([C@H](NS(=O)(=O)C3CCC3)C(F)(F)F)cn(C3CCOCC3)c2c1] |
| 527 | 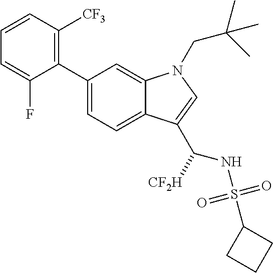 [SMILES: CC(C)(C)Cn1cc([C@H](NS(=O)(=O)C2CCC2)C(F)F)c2ccc(-c3c(F)cccc3C(F)(F)F)cc21] |
| 528 | 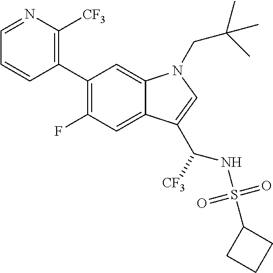 [SMILES: CC(C)(C)Cn1cc([C@H](NS(=O)(=O)C2CCC2)C(F)(F)F)c2cc(F)c(-c3cccnc3C(F)(F)F)cc21] |
| 529 | 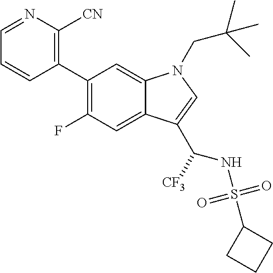 [SMILES: CC(C)(C)Cn1cc([C@H](NS(=O)(=O)C2CCC2)C(F)(F)F)c2cc(F)c(-c3cccnc3C#N)cc21] |
| 530 | 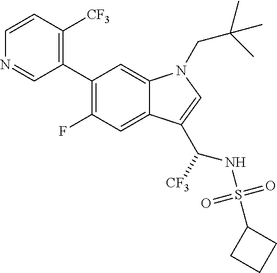 [SMILES: CC(C)(C)Cn1cc([C@H](NS(=O)(=O)C2CCC2)C(F)(F)F)c2cc(F)c(-c3cnccc3C(F)(F)F)cc21] |
| 533 | 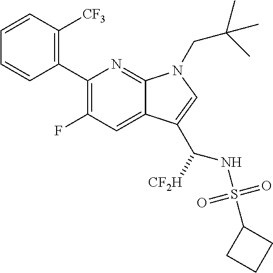 [SMILES: CC(C)(C)Cn1cc([C@H](NS(=O)(=O)C2CCC2)C(F)F)c2cc(F)c(-c3ccccc3C(F)(F)F)nc21] |
| 534 | 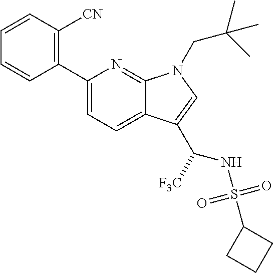 [SMILES: CC(C)(C)Cn1cc([C@H](NS(=O)(=O)C2CCC2)C(F)(F)F)c2ccc(-c3ccccc3C#N)nc21] |
| 535 | 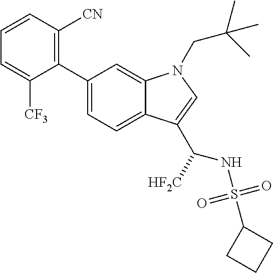 |
| 536 | 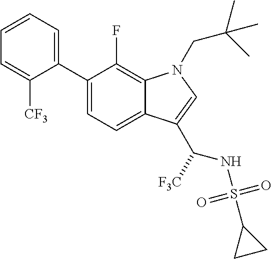 [SMILES: CC(C)(C)Cn1cc([C@H](NS(=O)(=O)C2CC2)C(F)(F)F)c2ccc(-c3ccccc3C(F)(F)F)c(F)c21] |
| 537 | 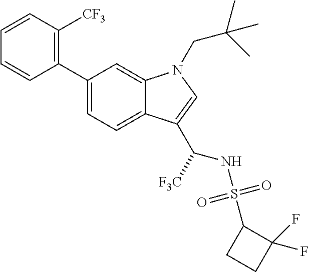 [SMILES: CC(C)(C)Cn1cc([C@H](NS(=O)(=O)C2CCC2(F)F)C(F)(F)F)c2ccc(-c3ccccc3C(F)(F)F)cc21] |
| 538 | 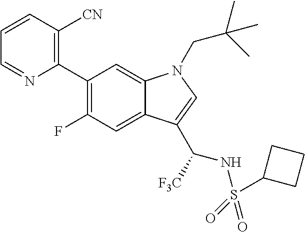 [SMILES: CC(C)(C)Cn1cc([C@H](NS(=O)(=O)C2CCC2)C(F)(F)F)c2cc(F)c(-c3ncccc3C#N)cc21] |
| 541 | 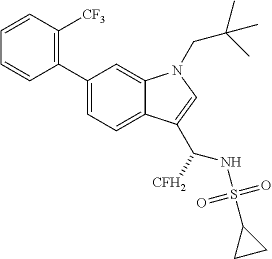 |
| 542 | 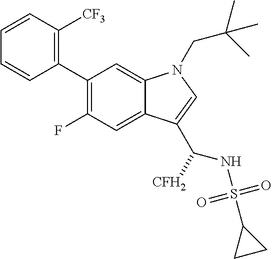 |
| 543 | 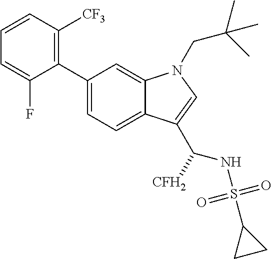 |
| 544 | 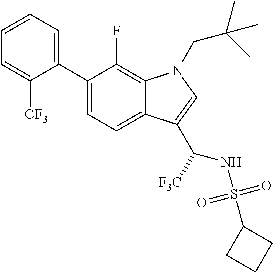 [SMILES: CC(C)(C)Cn1cc([C@H](NS(=O)(=O)C2CCC2)C(F)(F)F)c2ccc(-c3ccccc3C(F)(F)F)c(F)c21] |
| 545 | 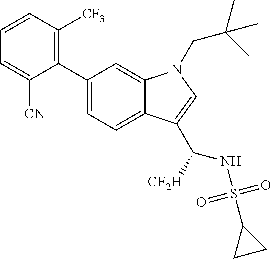 [SMILES: CC(C)(C)Cn1cc([C@H](NS(=O)(=O)C2CC2)C(F)F)c2ccc(-c3c(C#N)cccc3C(F)(F)F)cc21] |
| 546 | 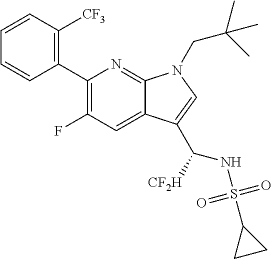 [SMILES: CC(C)(C)Cn1cc([C@H](NS(=O)(=O)C2CC2)C(F)F)c2cc(F)c(-c3ccccc3C(F)(F)F)nc21] |
| 547 | 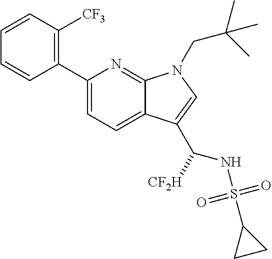 [SMILES: CC(C)(C)Cn1cc([C@H](NS(=O)(=O)C2CC2)C(F)F)c2ccc(-c3ccccc3C(F)(F)F)nc21] |
| 550 | 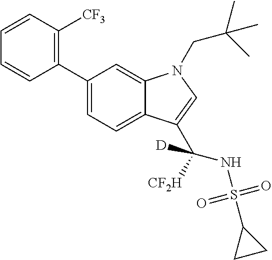 [SMILES: [2H][C@](NS(=O)(=O)C1CC1)(c1cn(CC(C)(C)C)c2cc(-c3ccccc3C(F)(F)F)ccc12)C(F)F] |
| 551 | 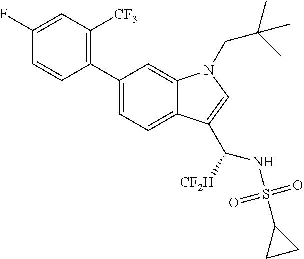 [SMILES: CC(C)(C)Cn1cc([C@H](NS(=O)(=O)C2CC2)C(F)F)c2ccc(-c3ccc(F)cc3C(F)(F)F)cc21] |
| 552 | 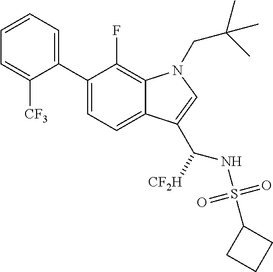 [SMILES: CC(C)(C)Cn1cc([C@H](NS(=O)(=O)C2CCC2)C(F)F)c2ccc(-c3ccccc3C(F)(F)F)c(F)c21] |
| 553 | 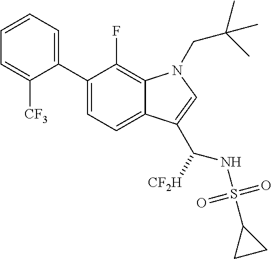 [SMILES: CC(C)(C)Cn1cc([C@H](NS(=O)(=O)C2CC2)C(F)F)c2ccc(-c3ccccc3C(F)(F)F)c(F)c21] |
| 555 | 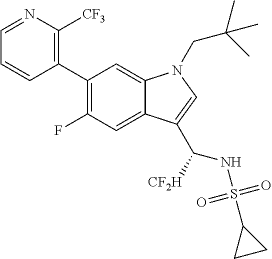 [SMILES: CC(C)(C)Cn1cc([C@H](NS(=O)(=O)C2CC2)C(F)F)c2cc(F)c(-c3cccnc3C(F)(F)F)cc21] |
| 557 | 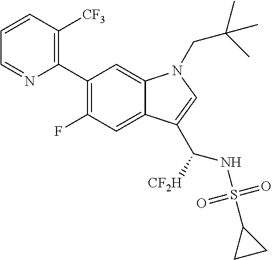 [SMILES: CC(C)(C)Cn1cc([C@H](NS(=O)(=O)C2CC2)C(F)F)c2cc(F)c(-c3ncccc3C(F)(F)F)cc21] |
| 558 | 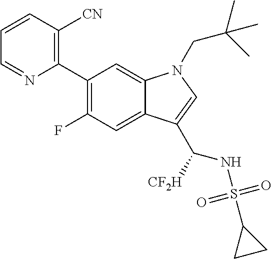 [SMILES: CC(C)(C)Cn1cc([C@H](NS(=O)(=O)C2CC2)C(F)F)c2cc(F)c(-c3ncccc3C#N)cc21] |
| 562 | 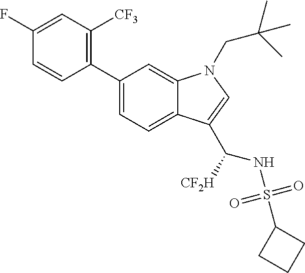 [SMILES: CC(C)(C)Cn1cc([C@H](NS(=O)(=O)C2CCC2)C(F)F)c2ccc(-c3ccc(F)cc3C(F)(F)F)cc21] |
| 563 | 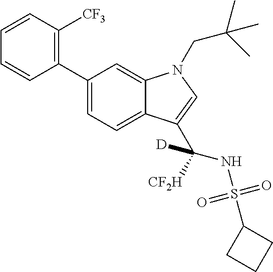 [SMILES: [2H][C@](NS(=O)(=O)C1CCC1)(c1cn(CC(C)(C)C)c2cc(-c3ccccc3C(F)(F)F)ccc12)C(F)F] |
| 564 | 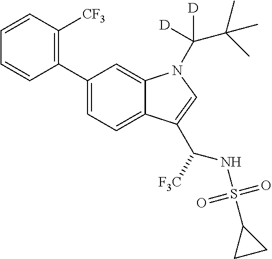 [SMILES: [2H]C([2H])(n1cc([C@H](NS(=O)(=O)C2CC2)C(F)(F)F)c2ccc(-c3ccccc3C(F)(F)F)cc21)C(C)(C)C] |
| 565 | 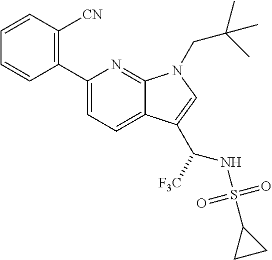 [SMILES: CC(C)(C)Cn1cc([C@H](NS(=O)(=O)C2CC2)C(F)(F)F)c2ccc(-c3ccccc3C#N)nc21] |
| 566 | 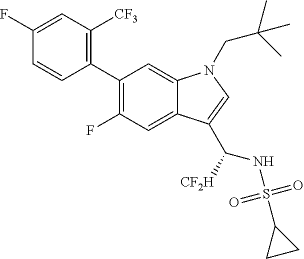 [SMILES: CC(C)(C)Cn1cc([C@H](NS(=O)(=O)C2CC2)C(F)F)c2cc(F)c(-c3ccc(F)cc3C(F)(F)F)cc21] |
| 567 | 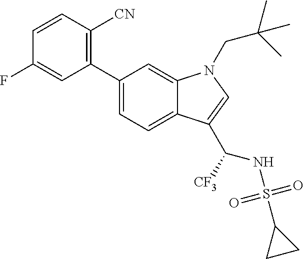 [SMILES: CC(C)(C)Cn1cc([C@H](NS(=O)(=O)C2CC2)C(F)(F)F)c2ccc(-c3cc(F)ccc3C#N)cc21] |
| 568 | 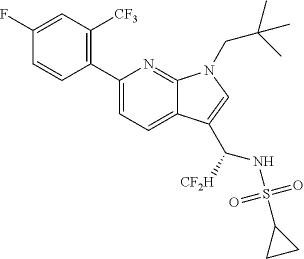 [SMILES: CC(C)(C)Cn1cc([C@H](NS(=O)(=O)C2CC2)C(F)F)c2ccc(-c3ccc(F)cc3C(F)(F)F)nc21] |

33. A pharmaceutical composition comprising a compound according to any one of claims 1 to 32 and a pharmaceutically acceptable carrier.

34. A method for treating a subject suffering from hyperglycemia, dyslipidemia, atherosclerosis, an autoimmune or inflammatory disorder or disease, or a cancer, comprising administering to the subject a therapeutically effective amount of a compound or pharmaceutically acceptable salt thereof according to any one of claims 1 to 32.

35. The method according to claim 34, wherein the cancer is one selected from the group consisting of glioblastoma, colorectal cancer, and hepatocellular carcinoma.

36. A method for repressing T_{H}17 cell development in a subject, comprising administering to the subject a compound or pharmaceutically acceptable salt thereof according to any one of claims 1 to 32.

37. A method for selectively agonizing REV-ERBα over REV-ERBβ in a subject, comprising administering to the subject a compound or pharmaceutically acceptable salt thereof according to any one of claims 1 to 32.

38. Use of a compound or pharmaceutically acceptable salt thereof according to any one of claims 1 to 32 for treating a subject suffering from hyperglycemia, dyslipidemia, atherosclerosis, an autoimmune or inflammatory disorder or disease, or a cancer.

39. The use according to claim 38, wherein the cancer is one selected from glioblastoma, colorectal cancer, and hepatocellular carcinoma.

40. Use of a compound or pharmaceutically acceptable salt thereof according to any one of claims 1 to 32 for repressing T_{H}17 cell development in a subject.

41. Use of a compound or pharmaceutically acceptable salt thereof according to any one of claims 1 to 32 for selectively agonizing REV-ERBα over REV-ERBβ in a subject.
